(19) United States
(12) Patent Application Publication
(10) Pub. No.: US 2025/0283871 A1
(43) Pub. Date: Sep. 11, 2025

(54) COMPOSITIONS AND METHODS FOR MODULATING THE INTERACTION BETWEEN SS18-SSX FUSION ONCOPROTEIN AND NUCLEOSOMES

(71) Applicants: Dana-Farber Cancer Institute, Inc., Boston, MA (US); The Trustees of Princeton University, Princetion, NJ (US)

(72) Inventors: Cigall Kadoch, Boston, MA (US); Matthew McBride, Boston, MA (US); Hai T. Dao, Memphis, TN (US); Tom W. Muir, Princetion, NJ (US); Nazar Mashtalir, Boston, MA (US)

(21) Appl. No.: 17/905,875

(22) PCT Filed: Jan. 21, 2021
(86) PCT No.: PCT/US2021/014367

§ 371 Date: Sep. 8, 2022

Related U.S. Application Data
(60) Provisional application No. 62/989,238, filed on Mar. 13, 2020.

(51) Int. Cl. G01N 33/50

(52) U.S. Cl. CPC G01N 33/5011; G01N 33/5091

(57) ABSTRACT

The present invention is based, in part, on the identification of a minimal region of the SS18-SSX fusion oncoprotein that mediates a direct, high-affinity interaction between the mSWI/SNF complex and the nucleosome acidic patch, and methods and agents of modulating the interaction between the SS18-SSX fusion protein and H2A K119Ub-marked nucleosomes to treat synovial sarcoma.

### CROSS-REFERENCE TO RELATED APPLICATIONS

This application is the U.S. national phase of International Patent Application No. PCT/US2021/014367, filed on 21 Jan. 2021, which claims the benefit of priority to U.S. Provisional Application Ser. No. 62/989,238, filed on 13 Mar. 2020; the entire contents of each of said applications are incorporated herein in its their entirety by this reference.

### STATEMENT OF RIGHTS

This invention was made with government support under grant number K99CA237855, 1DP2CA195762-01, 1R01 CA237241-01, 1U54 CA231638-01, R37-GM086868 and P01 CA196539 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 26, 2021, is named DFS-24825_SL.txt and is 1,165,513 bytes in size.

### BACKGROUND OF THE INVENTION

A synchronous combination of histone reader domains, chromatin complex conformations, DNA-binding transcription factors (TFs), and other features are required to orchestrate the appropriate targeting of chromatin regulatory machinery in eukaryotic cells. Chromatin reader proteins play critical roles in mediating the engagement of regulatory proteins and protein complexes to specific features of nucleosomal architecture, often to facilitate site-specific catalytic activities. These include bromodomains which recognize acetylated lysines (Fujisawa and Filippakopoulos (2017) Nat. Rev. Mol. Cell Biol. 18:246-262), PHD domains which recognize methylation and crotonylatation of histone tails (Hyun et al. (2017) Exp. Mol. Med. 49:e324; Xiong et al. (2016) Nat. Chem. Biol. 12:1111-1118), and increasingly appreciated, nucleosome acidic patch interacting domains of SNF2 helicase-based chromatin remodeling complexes (Dann et al. (2017) Nature 548:607-611; Levendosky and Bowman (2019) eLife 8, doi:10.7554/eLife.45472; Dao et al. (2019) Nat. Chem. Biol. doi:10.1038/s41589-019-0413-4). In parallel, TFs recognize their cognate DNA motifs genome-wide, and, when tethered to other proteins or protein complexes, such as chromatin remodeling complexes, can direct their global positioning on chromatin to achieve cell-, tissue- and cancer-specific gene expression programs. For example, TFs have been shown to tether transiently to the surfaces of mammalian SWI/SNF (BAF) ATP-dependent chromatin remodeling complexes to globally reposition them to sites enriched for specific TF DNA-binding motifs (Sandoval et al. (2018) Mol. Cell 71:554-566; Boulay et al. (2017) Cell 171:163-178). Importantly, results of recent large-scale human genetic sequencing studies indicate that perturbations across each of the above classes of chromatin-bound factors represent frequent and recurrent events in human cancer (Kadoch et al. (2013) Nat. Genet. 45:592-601; Valencia and Kadoch (2019) Nat. Cell Biol. 21:152-161; Kadoch and Crabtree (2015) Sci. Advances 1:e1500447), intellectual disability (Iwase et al. (2017) J. Neurosci. 37:10773-10782), and other disorders, with mutations ranging from point mutations and deletions to fusion proteins which alter target engagement and activity of chromatin regulatory complexes on the genome (Wan et al. (2017) Nature 543:265-269; McBride et al. (2018) Cancer Cell 33:1128-1141; Kadoch and Crabtree (2013) Cell 153:71-85).

It has remained elusive, however, how nuclear fusion oncoproteins that lack canonical TF DNA-binding or recognizable chromatin reader domains yield altered, region-specific targeting of chromatin regulatory proteins and protein complexes. For example, the SS18-SSX fusion oncoprotein involving the BAF complex subunit, SS18, and 78 amino acids of one of the SSX proteins normally expressed only in testes (Clark et al. (1994) Nat. Genet. 7:502-508; Crew et al. (1995) EMBO J. 14:2333-2340; De Leeuw et al. (1995) Hum. Mol. Genet. 4:1097-1099; Smith and McNeel (2010) Clinic. & Dev. Immunol. 2010:150591), is hallmark to 100% of cases of synovial sarcoma. Incorporation of SS18-SSX in to BAF complexes causes biochemical changes, such as the destabilization of the SMARCB1 (BAF47) subunit, and results in de novo BAF complex targeting to a highly cancer-specific set of sites, particularly, broad, polycomb-repressed regions at which polycomb complex occupancy is reduced and gene expression is activated (McBride et al. (2018) Cancer Cell 33:1128-1141). Although some studies have indicated SSX interactions with chromatin-associated factors (Banito et al. (2018) Cancer cell 33:527-541), the mechanism by which the site-specific binding and unique biochemical properties are achieved remains largely unknown.

Accordingly, there remains a great need in the art to identify therapeutic agents and methods that target SS18-SSX fusion oncoprotein to treat synovial sarcoma.

### SUMMARY OF THE INVENTION

The present invention is based, at least in part, on the identification of a minimal region of the SS18-SSX fusion oncoprotein, the hallmark oncogenic driver of synovial sarcoma (SS), that mediates a direct, high-affinity interaction between the mSWI/SNF complex and the nucleosome acidic patch. This engagement results in altered mSWI/SNF composition and orientation on nucleosomes, driving cancer-specific mSWI/SNF complex targeting and gene expression. Furthermore, an acidic C-terminal region of SSX confers preferential affinity to repressed, H2AK119Ub-marked nucleosomes, underlying the selective targeting to polycomb-marked genomic regions and SS-specific dependency on PRC1 function. Together, these results describe a functional interplay between a key nucleosome binding hub and a histone modification that underlies the disease-specific chromatin recruitment of a major chromatin remodeling complex.

Accordingly, in one aspect, a method of treating a subject afflicted with synovial sarcoma comprising administering to the subject a therapeutically effective amount of an agent that inhibits binding of a SS18-SSX fusion protein to a nucleosome, optionally wherein the nucleosome is an H2A K119Ub-marked nucleosome, is provided.

Numerous embodiments are further provided that can be applied to any aspect of the present invention and/or combined with any other embodiment described herein. For example, in one embodiment, the SS18-SSX fusion protein comprises a C-terminal region containing a basic region, and an acidic region of a SSX protein, optionally wherein the basic region comprises a minimal 34-amino acid region. In another embodiment, the SS18-SSX fusion protein is selected from Table 2. In still another embodiment, the agent inhibits binding of the basic region of the SS18-SSX fusion protein to an acidic patch of the nucleosome, optionally wherein the nucleosome is an H2A K119Ub-marked nucleosome. In yet another embodiment, the agent is a small molecule inhibitor, a small molecule degrader, CRISPR guide RNA (gRNA), RNA interfering agent, oligonucleotide, peptide or peptidomimetic inhibitor, aptamer, antibody, or intrabody. In another embodiment, the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA). In still another embodiment, the agent comprises an antibody and/or intrabody, or an antigen binding fragment thereof, which specifically binds to the SS18-SSX fusion protein, the SSX tail, and/or the H2AK119Ub-marked nucleosome, optionally wherein the SSX tail is SSX tail (34 amino acid) and/or SSX tail (78 amino acid). In yet another embodiment, the agent comprises an antibody and/or intrabody, or an antigen binding fragment thereof, which specifically binds to at least one of the following regions: (1) the basic region of the SS18-SSX fusion protein; (2) the acidic region of the SS18-SSX fusion protein; (3) the acidic patch of the H2AK119Ub-marked nucleosome; and/or (4) the H2AK119Ub mark. In another embodiment, the antibody and/or intrabody, or antigen binding fragment thereof, is chimeric, humanized, composite, or human. In still another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments. In yet another embodiment, the agent induces deletion or mutation of the basic region of the SS18-SSX fusion protein, the acidic region of the SS18-SSX fusion protein, the acidic patch of the H2AK119Ub-marked nucleosome, and/or a region within the SSX tail (34 amino acid). In another embodiment, the agent inhibits H2A ubiquitination. In still another embodiment, the agent inhibits ubiquitin ligase activity of a PRC1 complex. In yet another embodiment, the agent reduces expression, copy number, and/or ubiquitin ligase activity of RING1A and/or RING1B. In another embodiment, the agent inhibits recruitment of a SS18-SSX fusion protein-bound BAF complex to an H2AK119Ub-marked nucleosome. In still another embodiment, the agent inhibits activation of at least one oncogenic target gene of the SS18-SSX fusion protein. In yet another embodiment, the oncogenic target gene of the SS18-SSX fusion protein is selected from the group consisting of WNT16 and oncogenic target genes listed in McBride et al. (2018) Cancer Cell 33:1128-1141. In another embodiment, the agent reduces the number of viable or proliferating cells in the cancer, and/or reduces the volume or size of a tumor comprising the cancer cells. In still another embodiment, the method further comprises administering to the subject an immunotherapy and/or cancer therapy, optionally wherein the immunotherapy and/or cancer therapy is administered before, after, or concurrently with the agent. In yet another embodiment, the immunotherapy is cell-based. In another embodiment, the immunotherapy comprises a cancer vaccine and/or virus. In still another embodiment, the immunotherapy inhibits an immune checkpoint, such as an immune checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In yet another embodiment, the cancer therapy is selected from the group consisting of radiation, a radiosensitizer, and a chemotherapy. In another embodiment, the method further comprises administering to the subject at least one additional therapeutic agent or regimen for treating the cancer.

In another aspect, a method of reducing viability or proliferation of synovial sarcoma cells comprising contacting the synovial sarcoma cells with an agent that inhibits binding of a SS18-SSX fusion protein to a nucleosome, optionally wherein the nucleosome is an H2AK119Ub-marked nucleosome, is provided.

As described above, numerous embodiments are further provided that can be applied to any aspect of the present invention and/or combined with any other embodiment described herein. For example, in one embodiment, the SS18-SSX fusion protein comprises a C-terminal region containing a basic region, and an acidic region of a SSX protein, optionally wherein the basic region comprises a minimal 34-amino acid region. In another embodiment, the SS18-SSX fusion protein is selected from Table 2. In still another embodiment, the agent inhibits binding of the basic region of the SS18-SSX fusion protein to an acidic patch of the H2AK119Ub-marked nucleosome. In yet another embodiment, the agent is a small molecule inhibitor, a small molecule degrader, CRISPR guide RNA (gRNA), RNA interfering agent, oligonucleotide, peptide or peptidomimetic inhibitor, aptamer, antibody, or intrabody. In another embodiment, the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA). In still another embodiment, the agent comprises an antibody and/or intrabody, or an antigen binding fragment thereof, which specifically binds to the SS18-SSX fusion protein, or the H2AK119Ub-marked nucleosome. In yet another embodiment, the agent comprises an antibody and/or intrabody, or an antigen binding fragment thereof, which specifically binds to at least one of the following regions: (1) the basic region of the SS18-SSX fusion protein; (2) the acidic region of the SS18-SSX fusion protein; (3) the acidic patch of the H2AK119Ub-marked nucleosome; and/or (4) the H2AK119Ub mark. In another embodiment, the antibody and/or intrabody, or antigen binding fragment thereof, is chimeric, humanized, composite, or human. In still another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments. In yet another embodiment, the agent induces deletion or mutation of the basic region of the SS18-SSX fusion protein, the acidic region of the SS18-SSX fusion protein, and/or the acidic patch of the H2AK119Ub-marked nucleosome. In another embodiment, the agent inhibits H2A ubiquitination. In still another embodiment, the agent inhibits ubiquitin ligase activity of a PRC1 complex. In yet another embodiment, the agent reduces expression, copy number, and/or ubiquitin ligase activity of RING1A and/or RING1B. In another embodiment, the agent inhibits recruitment of a SS18-SSX fusion protein-bound BAF complex to an H2AK119Ub-marked nucleosome. In still another embodiment, the agent inhibits activation of at least one oncogenic target gene of the SS18-SSX fusion protein. In yet another embodiment, the oncogenic target gene of the SS18-SSX fusion protein is selected from the group consisting of WNT16 and oncogenic target genes listed in McBride et al. (2018) Cancer Cell 33:1128-1141. In another embodiment, the method further comprises contacting the cancer cells with an immunotherapy and/or cancer therapy, optionally wherein the immunotherapy and/or cancer therapy is administered before, after, or concurrently with the agent. In still another embodiment, the immunotherapy is cell-based. In yet another embodiment, the immunotherapy comprises a cancer vaccine and/or virus. In another embodiment, the immunotherapy inhibits an immune checkpoint, such as an immune checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In still another embodiment, the cancer therapy is selected from the group consisting of radiation, a radiosensitizer, and a chemotherapy.

In still another aspect, a method of assessing the efficacy of an agent for treating synovial sarcoma in a subject, comprising: a) detecting in a subject sample at a first point in time the number of viable and/or proliferating cancer cells; b) repeating step a) during at least one subsequent point in time after administration of the agent; and c) comparing number of viable and/or proliferating cancer cells detected in steps a) and b), wherein the absence of, or a significant decrease in number of viable and/or proliferating cancer cells in the subsequent sample as compared to the amount in the sample at the first point in time, indicates that the agent treats synovial sarcoma in the subject, is provided.

As described above, numerous embodiments are further provided that can be applied to any aspect of the present invention and/or combined with any other embodiment described herein. For example, in one embodiment, the subject has undergone treatment, completed treatment, and/or is in remission for synovial sarcoma between the first point in time and the subsequent point in time. In another embodiment, the first and/or at least one subsequent sample is selected from the group consisting of ex vivo and in vivo samples. In still another embodiment, the first and/or at least one subsequent sample is obtained from an animal model of synovial sarcoma. In yet another embodiment, the first and/or at least one subsequent sample is a portion of a single sample or pooled samples obtained from the subject. In another embodiment, the sample comprises cells, serum, peritumoral tissue, and/or intratumoral tissue obtained from the subject. In still another embodiment, the method further comprises determining responsiveness to the agent by measuring at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria. In yet another embodiment, the agent is administered in a pharmaceutically acceptable formulation. In another embodiment, the step of administering or contacting occurs in vivo, ex vivo, or in vitro.

In yet another aspect, a cell-based assay for screening for agents that reduce viability or proliferation of a synovial sarcoma cell comprising: a) contacting the synovial sarcoma cell with a test agent; and b) determining the ability of the test agent to inhibit binding of a SS18-SSX fusion protein, a SSX (78 amino acid) region, and/or a SSX (34 amino acid) minimal region to a nucleosome, optionally wherein the nucleosome is a H2AK119Ub-marked nucleosome, is provided.

As described above, numerous embodiments are further provided that can be applied to any aspect of the present invention and/or combined with any other embodiment described herein. For example, in one embodiment, the SS18-SSX fusion protein comprises a C-terminal region containing a basic region, and an acidic region of a SSX protein, optionally wherein the basic region comprises a minimal 34-amino acid regbion. In another embodiment, the SS18-SSX fusion protein is selected from Table 2. In still another embodiment, the step of contacting occurs in vivo, ex vivo, or in vitro. In yet another embodiment, the assay further comprising determining the ability of the test agent to inhibit recruitment of a SS18-SSX fusion protein-bound BAF complex to an H2AK119Ub-marked nucleosome and/or H2AK 119Ub-marked region of chromatin in cells, optionally wherein the cellular chromatin comprises a PRC1/H2A Ub domain. In another embodiment, the assay further comprises determining the ability of the test agent to inhibit activation of at least one oncogenic target gene of the SS18-SSX fusion protein. In still another embodiment, the oncogenic target gene of the SS18-SSX fusion protein is selected from the group consisting of WNT16 and oncogenic target genes listed in McBride et al. (2018) Cancer Cell 33:1128-1141. In yet another embodiment, the assay further comprises determining a reduction in the viability or proliferation of the cancer cells.

In another aspect, an in vitro assay for screening for agents that reduce viability or proliferation of a synovial sarcoma cell comprising: a) mixing a protein comprising a c-terminal basic region and a c-terminal acidic region of a SSX protein and a nucleosome together, optionally wherein the nucleosome is a H2AK119Ub-marked nucleosome; b) adding a test agent to the mixture; and c) determining the ability of the test agent to decrease binding of the protein to the nucleosome, is provided.

As described above, numerous embodiments are further provided that can be applied to any aspect of the present invention and/or combined with any other embodiment described herein. For example, in one embodiment, the protein comprises c-terminal 34 amino acids (aa155-188) of a SSX protein. In another embodiment, the protein comprises c-terminal 78 amino acids (aa 111-188) of a SSX protein. In still another embodiment, the protein is a SS18-SSX fusion protein. In yet another embodiment, the SS18-SSX fusion protein is selected from Table 2. In another embodiment, the SS18-SSX fusion protein comprises SS18 protein fused with a c-terminal portion of a SSX protein. In still another embodiment, the SS18-SSX fusion protein comprises c-terminal 34 amino acids (aa155-188) of a SSX protein. In yet another embodiment, the SS18-SSX fusion protein comprises c-terminal 78 amino acids (aa 111-188) of a SSX protein. In another embodiment, the SSX protein is selected form the group consisting of human SSX1, SSX2, SSX3, SSX4, SSX6, SSX7, SSX8, and SSX9. In still another embodiment, the SS18-SSX fusion protein comprises W164, R167, L168, R169 and/or R171 of SEQ ID: 3, 7, 13, 17, 21, 25, or 31, or orthologs thereof. In yet another embodiment, the SS18-SSX fusion protein is a part of a BAF complex. In another embodiment, the nucleosome comprises H2A protein comprising E56, E64, D90, E91, E92 and/or E113 of human, mouse, rat, or Xenopus H2A, or orthologs thereof; and/or H2B protein comprising E105 and/or E113 of human, mouse, rat, or Xenopus H2B, or orthologs thereof. In still another embodiment, the subject is an animal model of the cancer, optionally wherein the animal model is a mouse model. In yet another embodiment, the subject is a mammal. In another embodiment, the mammal is a mouse or human. In still another embodiment, the mammal is a human.

In still another aspect, an isolated modified protein complex selected from the group consisting of protein complexes listed in Table 3, wherein the isolated modified protein complex comprises at least one subunit that is modified, is provided.

As described above, numerous embodiments are further provided that can be applied to any aspect of the present invention and/or combined with any other embodiment described herein. For example, in one embodiment, the at least one modified subunit is a fragment of the subunit. In another embodiment, the fragment of the subunit binds to at least one binding partner of the subunit to form the isolated modified protein complex. In still another embodiment, the fragment of the subunit comprises the basic region and/or the acidic region of a SSX protein. In yet another embodiment, the fragment of the subunit comprises c-terminal 34 amino acids (aa155-188) of a SSX protein. In another embodiment, the fragment of the subunit comprises c-terminal 78 amino acids (aa 111-188) of a SSX protein. In still another embodiment, the SSX protein is selected form the group consisting of human SSX1, SSX2, SSX3, SSX4, SSX6, SSX7, SSX8, and SSX9. In yet another embodiment, the fragment of the subunit comprises the acidic patch of a nucleosome and/or the H2A K119 Ub mark. In another embodiment, at least one subunit is linked to at least another subunit. In still another embodiment, at least one subunit is linked to at least another subunit through covalent cross-links. In yet another embodiment, at least one subunit is linked to at least another subunit through a peptide linker. In another embodiment, the at least one subunit comprises a heterologous amino acid sequence. In still another embodiment, the heterologous amino acid sequence comprises an affinity tag or a label. In yet another embodiment, the affinity tag is selected from the group consisting of Glutathione-S-Transferase (GST), calmodulin binding protein (CBP), protein C tag, Myc tag, HaloTag, HA tag, Flag tag, His tag, biotin tag, and V5 tag. In yet another embodiment, the label is a fluorescent protein. In another embodiment, the at least one subunit is selected from the group consisting of HA-SS18-SSX1, V5-SS18-SSX1, V5-SS18-SSX1 34aa tail, V5-SS18-SSX1 78aa tail, H2A, and H2B.

In yet another aspect, a pharmaceutical composition comprising an isolated modified protein complex described herein, and a carrier, is provided.

### BRIEF DESCRIPTION OF THE DRAWINGS

FIG. 1A-FIG. 1E show that SS18-SSX-containing BAF complexes exhibit significantly increased affinity for chromatin. FIG. 1A shows colloidal blue staining performed on purifications of wild-type BAF complexes (from HA-SS18 WT-expressing 293T cells) and SS18-SSX-containing BAF complexes (from HA-SS18-SSX1-expressing cells), from soluble nuclear extract (NE) and chromatin-bound (CHR) fractions. Equal amounts (by volume) of nuclei in each condition were isolated and subsequently purified in to NE and CHR fractions. FIG. 1B shows MS spectral counts for BAF complex subunits (green) and histone proteins (orange) from HA-SS18 WT and HA-SS18-SSX purifications from NE and CHR fractions in (FIG. 1A). Peptides counts are log 2 normalized to bait (SS18 peptides). FIG. 1C shows density sedimentation gradients using 10-30% glycerol performed on HA-SS18 WT and HA-SS18-SSX1 purifications from HEK-293T cells. BAF complex subunits and histone proteins are indicated. SYPRO® Ruby staining was used for visualization. FIG. 1D shows immunoblot for SMARCA4 and SMARCC1 performed on Aska SS cells in shCtrl (control, non-targeting harpin shRNA) and shSSX (shRNA targeted to SSX) conditions following differential salt extraction (0-1000 mM NaCl). FIG. 1E shows FRAP studies performed on HEK293T cells expressing either GFP-SS18 WT or GFP-SS18-SSX1. Recovery kinetics were recorded and the recovery half-times were calculated to be 10.1 and 33.9 seconds for GFP-SS18 WT and GFP-SS18-SSX1, respectively (values represent mean of n=30 cells per condition, with error bars indicating standard deviation at each time point).

FIG. 2A-FIG. 2H show that SS18-SSX-containing BAF complexes exhibit high-affinity interactions with histones and longer residency times on chromatin. FIG. 2A shows MS spectral counts for BAF complex subunits and histone proteins from HA-SS18 WT and HA-SS18-SSX purifications from soluble nuclear extract NE and CHR fractions from FIG. 1A. Total number of peptides (number of peptides normalized to bait, SS18) are shown. FIG. 2B shows ranked peptides captured in HA-SS18-SSX purification (chromatin-bound fraction). Red indicates mSWI/SNF complex subunits. Green indicates histones. Orange indicates members of PRC1 and PRC2 complexes, shown for comparison. See also Tables 5A-5E. FIG. 2C has two panels. The top panel shows immunoblot for GFP and H2A performed on HEK-293T cells infected with either GFP-SS18 WT or GFP-SS18-SSX following differential salt extraction (0-1000 mM NaCl). The bottom panel shows immunoblot for SS18 and H2A K119Ub performed on HEK-293T cells (naive) and Aska-SS cells following differential salt extraction (0-1000 mM NaCl) experiments. FIG. 2D shows immunoblot for SMARCA4 and SS18 performed HEK-293T cells or Aska SS cells (SS18-SSX+) following differential salt extraction (0-1000 mM NaCl). FIG. 2E shows FRAP experiments performed in the Aska SS cell line modified to express BRG1 (SMARCA4)-Halo. Aska-SS cells were treated with either shControl shRNA hairpin or shSSX (targeting the SS18-SSX fusion). Recovery t½ times (seconds) with 95% CIs are shown; n=20 cells. FIG. 2F shows SYPRO® Ruby staining indicating identified proteins from FIG. 1c in Fraction 13 (HA-SS18 WT) and Fraction 18 (HA-SS18-SSX). FIG. 2G shows SMARCB1 peptide abundance (normalized to SMARCA4) and relative to SS18 WT-bound complexes (soluble NE fraction). NE indicates nuclear extract; CHR indicates chromatin-bound fraction. FIG. 2H has two panesl. The left panel shows cyber-gold staining of complexes purified from untreated (no benzonase) nuclear extracts isolated via ammonium sulfate extraction. The right panel shows that H3 immunoblot reveals prominent histone binding in HA-SS18-SSX-bound complexes but not in HA-SS18 WT-bound complexes.

FIG. 3A-FIG. 3I show that conserved basic and acidic regions within a minimal SSX domain are necessary and sufficient to bind nucleosomes and promote specialized BAF complex chromatin recruitment and activity. FIG. 3A shows GST (control) and GST-SSX1 (78aa) purified recombinant proteins incubated with mammalian mononucleosomes (purified by MNase digestion), captured using glutathione resin, visualized using colloidal blue. FIG. 3B shows quantitative targeted MS analysis of MBP pull down experiments using the MBP-SSX 78aa protein and endogenous mammalian nucleosomes purified using MNase digestion from 293T cells. Log 2 (FC) calculated relative to input sample. Red indicates enriched; blue indicates depleted. FIG. 3C shows immunofluorescence analysis of V5-tagged SS18 and SS18-SSX relative to RING1B and SUZ12 in 293T cells. Arrows indicate positions of the Barr bodies (inactive X). Scale bar indicates 5 μm. FIG. 3D shows alignment of SSX1 protein across species and relative to related PRDM7/9 proteins. Highly conserved basic and acidic regions are indicated in blue and red, respectively. FIG. 3D discloses SEQ ID NOS 231-233, 233-236, 232, 237, 232 and 238-243, respectively, in order of appearance. FIG. 3E shows pull-down experiments of N-terminally biotinylated SSX peptides (scrambled (aa155-188), SSX 34aa (aa155-188), SSX 24aa (aa164-188) and SSX 23aa (aa165-188) incubated with mammalian mononucleosomes and visualized with colloidal blue. FIG. 3F shows pull-down experiments of N-terminally biotinylated SSX peptides including scrambled control, wildtype (WT) and mutant variants (single alanine substitutions as well as regional substitutions (i.e., Basic/A, basic region RLRERK-->AAAAAA (SEQ ID NOS 219-220, respectively, in order of appearance); Acidic/A, acidic region DPEEDDE-->AAAAAAA (SEQ ID NOS 221-222, respectively, in order of appearance)) incubated with mammalian mononucleosomes and visualized with colloidal blue. FIG. 3F discloses SEQ ID NO: 232. FIG. 3G shows ChIP-seq density heatmaps reflecting chromatin occupancy of V5-SS18-SSX1, V5-SS18, V5-SS18-SSX (24aa) and V5-SS18-SSX (34aa) over all V5 Peaks (38,014 total peaks). FIG. 3H shows heatmap reflecting top 5% upregulated and downregulated genes (Z-score) by RNA-seq for each condition. FIG. 3I shows proliferation experiments performed on SYO-1 SS cells infected with either control hairpin (shCt) or shSSX (knockdown of endogenous SS18-SSX) with overexpression of empty vector control, SS18-SSX 78aa or SS18-SSX 34aa variants. n=3 independent experimental replicates; error bars represent standard deviation; ** indicate p<0.01.

FIG. 4A-FIG. 4H show the SSX 78aa protein binds mononucleosomes, with preference for nucleosomes decorated with repressive histone modifications. FIG. 4A shows coomassie-stained gel of recombinantly purified GST, GST-SSX (78aa) proteins, run next to BSA protein as control. FIG. 4B shows purification of mammalian mononucleosomes from HEK-293T cells using MNase digestion. FIG. 4C shows incubation of GST or GST-SSX (78aa) with either recombinant or mammalian mononucleosomes, resolved by immunoblot for GST and histone H3 or Coomassie and histone H3. Two representative experiments are shown. FIG. 4D shows purification of MBP and MBP-SSX (78aa) proteins for targeted, quantitative histone mass-spectrometry. Quantitative histone mass spectrometry performed on MBP-SSX1 (versus MBP control) incubated with pooled mononucleosomes isolated from HEK-293T cells via MNase digestion. Experiment performed in n=2 replicates. See also Tables 3A-3C. FIG. 4E shows a schematic diagram for targeted MS experiments. FIG. 4F shows enrichment of SSX-bound histone peptides, over input. Enriched and depleted proteins are shown in red and blue, respectively. FIG. 4G shows quantitative densitometry normalized to input reflecting GST-SSX 78aa preferential binding to mammalian mononucleosomes (prepared via MNase digestion in HEK-293T cells) versus recombinant, unmodified nucleosomes. Bars represent averages of n=3 independent experiments, error bars represent standard deviation; p-value=0.0164. FIG. 4H shows heatmap reflecting enrichment or depletion of selected histone marks, including H2AZ and H3K4 methylation states. Scale=log 2FC.

FIG. 5A-FIG. 5G show nucleosome binding and nuclear localization properties of SS18-SSX and SSX variants. FIG. 5A shows immunofluorescence imaging performed on IMR90 fibroblasts and HEK293T cells infected with either V5-SS18-SSX or V5-SS18. Visualized in red for H3K9me3, SMARCA4, PBRM1, SMARCC1, H3K9Ac across experiments. DAPI is shown as nuclear stain and merged images are provided with scale bars; Scale bar indicates 5 μm. FIG. 5B shows IF-based localization of SS18 FL (1-188aa) in fibroblasts. H2AUb119, DAPI counterstain, and merged images are shown. Scale bar indicates 5 μm. FIG. 5C shows peptide competition experiment using Biotinylated SSX peptide (aa 155-188) and unlabeled SSX (aa 155-188). Visualization for Histone H3 uses immunoblot. FIG. 5D shows SSX peptide hybridization experiments performed on methanol-fixed cells. Streptavidin (SA) used for biotinylated SSX peptide visualization, H2AUb119 for Barr bodies. DAPI counterstain and merged images shown. Scale bar indicates 5 μm. FIG. 5E has two panels. The top panel shows conservation analysis among SSX and PRDM 7/9 human protein regions. The bottom panel shows peptide pull down experiments with recombinant nucleosomes performed with Scrambled control SSX1, SSX1, PRDM7, PRDM9. Visualization is by colloidal blue staining. FIG. 5E discloses SEQ ID NOS 232-233, 236-237, 244 and 224, respectively, in order of appearance. FIG. 5F has two panels. The left panel shows alignment of SSX proteins (SSX 1-9). The right panel shows peptide pull down experiments with recombinant nucleosomes performed with aa 155-188 of SSX family members. Visualization is by colloidal blue staining. FIG. 5F discloses SEQ ID NOS 232, 232, 232-233 and 233-237, respectively, in order of appearance. FIG. 5G shows peptide competition experiment using Biotinylated SSX peptide (aa 155-188) and Scrambled control SSX peptide (aa 155-188). Visualization for Histone H3 is by immunoblot.

FIG. 6A-FIG. 6E show defining a minimal 34-aa SSX region responsible for chromatin engagement and oncogenic gene expression. FIG. 6A shows additional representative V5 ChIP-seq and RNA-seq tracks, here shown at the SOX2 and GALNT9 loci. FIG. 6B shows differential salt experiments ([0-1000 mM NaCl]) performed on HEK-293T cells infected with either SS18-SSX 34aa versus SS18-SSX 24aa. Immunoblots for V5 as well as GAPDH and H3 (controls) are shown. FIG. 6C shows immunofluoroscence imaging of IMR90 fibroblasts infected with SS18 and SS18-SSX variants, as indicated, and stained for V5 (SS18-SSX or SSX variant) and DAPI; merged images are shown. Localization to H2AUb119-high sites (Barr bodies) is highlighted. Scale bar indicates 5 μm. FIG. 6D shows beta-gal senescence assay performed on IMR90 cells infected with WT SS18, SS18-SSX and SSX FL and 78aa variants, as indicated. FIG. 6E shows that SYO-1 synovial sarcoma cells were treated with either shCtrl (control hairpin) or shSSX (shRNA targeting SSX) to reduce levels of endogenous fusion, followed by rescue of SS18-SSX WT and mutant variants or empty vector control. Proliferation was evaluated over 16 days (see also FIG. 3I).

FIG. 7A-FIG. 7J show that the SSX basic region outcompetes the SMARCB1 C-terminal alpha-helical domain for nucleosome acidic patch binding. FIG. 7A shows incubation of biotinylated SSX peptides (aa 155-188) in either WT or RLR motif-mutant forms (R167A, R169A, R171A) with nucleosomes. FIG. 7B shows photocrosslinking experiments performed with reactive diazarine probes localized throughout the nucleosome acidic patch region indicate strongest binding to H2A E56 and 12B E113 residues. FIG. 7C shows SSX binding sites mapped on nucleosome PDB: 1KX5. Acidic patch crosslinked sites are labeled. FIG. 7D shows incubation of GST-SSX 78aa tail with either WT or acidic patch mutant nucleosomes (D90N, E92K, and E113K). Visualization of binding is by histone H3 immunoblot. FIG. 7E shows LANA peptide competition experiment with SSX 34aa biotinylated peptide bound to nucleosomes. FIG. 7F shows TALOS secondary structure prediction of the SSX 78aa region. An alpha helical probability (aa HAWTHRLRERK (SEQ ID NO: 223)) is indicated in red. The protein is largely disordered with a short helical--like segment (aa164-171) and a beta-strand like segment (aa174-179). FIG. 7F discloses SEQ ID NO: 232. FIG. 7G shows V5 ChIP-seg heat map reflecting genome-wide localization of V5-tagged SS18-SSX, SS18 WT and SS18-SSX RLR-->RLA (RI69A) mutant in CRL7250 fibroblasts. FIG. 7H shows reciprocal competition experiments performed with either SMARCB1 C-terminal alpha helical domain bound to nucleosomes or SSX 34aa bound to nucleosomes and competed with indicated peptide. FIG. 7I shows REAA nucleosome remodeling assay performed with BAF complexes containing either WT SS18 or SS18-SSX. Experiment performed at 37 degrees C., 0-40 min time course, BAF complex capture performed using ARDD1A IP. FIG. 7J shows ATAC-seq DNA accessibility (log 2FC(RPKM+1) performed in CRL7250 fibroblasts over SS18-SSX-specific sites and SS18 WT/SS18-SSX shared sites, defined in FIG. 7G.

FIG. 8A-FIG. 8G show that the SSX basic region and SMARCB1 C-terminal alpha helical domain compete for nucleosome acidic patch binding. FIG. 8A shows strategy for nucleosome-peptide photocrosslinking. FIG. 8B shows additional (replicate) photocrosslinking experiments performed with reactive diazarine probes localized throughout the nucleosome acidic patch region indicate strongest binding to H2A E56 and H2B E113 residues, weaker binding to H2A E91, and no binding to E61, E92, and D90 residues. Experimental conditions are as follows: 0.3 μM mononucleosomes, 3 μM SSX, 150 mM KCl. FIG. 8C shows pulldown experiments performed with either Scrambled or SSX 34aa peptides (biotinylated) incubated with mammalian mononucleosomes prepared from cells infected with WT H2A, or H2AD90N, H2A E92K mutant variants. FIG. 8D shows 15N-HSQC spectrum of SSX1 mutant having 7 C-terminal residue deletion, with assignments marked in red. The data were collected using 330 IM protein in pH 6.5 buffer at 15° C. on a 700 MHz spectrometer. FIG. 8E shows a model indicating docking of solved LANA peptide-nucleosome binding region and SSX peptide crosslinking in the nucleosome acidic patch. Interacting residues from photocrosslinking are highlighted. FIG. 8F shows modeling of SSX C-term (34aa) alpha helical peptide on nucleosome structure (PDB: 1KX5) using ZDOCK, in full nucleosome and zoomed-in view of acidic patch region. FIG. 8G shows photocrosslinking experiments performed with SSX 34aa peptide incubated with nucleosomes modified at the H2A E56 residue, with and without LANA peptide competition.

FIG. 9A-FIG. 9G show that mutations in the basic region of SSX affect the targeting and function of SS18-SSX-containing BAF complexes. FIG. 9A shows gene expression changes across each SS18 WT and SS18-SSX variant conditions from FIG. 7G. FIG. 9B shows proliferative rescue experiments performed in SYO-1 SS cell line treated with shSSX and rescued with either vector control, SS18-SSX or SS18-SSX (R169A or W164A) variants. n=3 independent experiments performed; error bars represent standard deviation; * indicates p<0.05. FIG. 9C shows peptide hybridization of IMR90 cells using SSX and mutant basic region mutant peptides. Arrows indicate positions of the Barr bodies. Scale bar indicates 5 μm. FIG. 9C discloses SEQ ID NOS 221-222, respectively, in order of appearance. FIG. 9D shows immunoblot performed on whole-cell extracts (RIPA extraction) from SYO1 cells treated with either shCtrl or shSSX and infected with either empty vector or SS18-SSX variants, used in proliferation experiments in FIG. 9B. FIG. 9E shows peptide hybridization of IMR90 cells using SSX and mutant basic region mutant (W164A and R169A) peptides. Arrows indicate positions of the Barr bodies. Scale bar indicates 5 μm. FIG. 9F shows ChIP-seq studies (anti-V5) performed in CRL7250 cells infected with either SS18-SSX or SS18-SSX W164A mutant, mapped as summary plot over SS18-SSX target sites. FIG. 9G shows RNA-seq (gene expression) data, box and whisker plots indicating average expression in SS18-SSX versus SS18-SSX W164A mutant conditions.

FIG. 10A-FIG. 10G show subunit composition, chromatin binding, and functional properties of SS18-SSX-bound BAF complexes. FIG. 10A shows SMARCB1 peptide abundance calculated from MS experiments (anti-SMARCA4 (BRG1) IPs) performed in Aska-SS synovial sarcoma cells, human Fibroblasts, and HEK-293T cells. Peptide abundance normalized to SMARCA4 abundance. FIG. 10B shows input and GFP IPs performed in Aska-SS cells infected with either GFP-SS18 or GFP-SS18-SSX. SMARCC1, SS18, GFP, SMARCB1, and TBP levels are shown. FIG. 10C shows SS18-SMARCA4 crosslinks detected in CX-MS experiments of intact, fully-formed BAF complexes in (Mashtalir et al. (2018) Cell 175:1272-1288). FIG. 10D shows immunoblot studies performed on CRL7250 cells infected with SS18-SSX variants indicated. FIG. 10E shows the immunoblot performed for ARID1A and SS18 on complexes captured via ARID1A, used for nucleosome remodeling and ATPase assays. FIG. 10F shows ATAC-seq experiments performed in SYO-1 SS cells in shCtrl and shSSX conditions, mapped over SS18 ChIP-seq. FIG. 10G shows ATPase activity calculated by ADP-Glo for SS18 WT- and SS18-SSX-containing BAF complexes. T indicates 0-40 min timecourse; n=3 experimental replicates at each time point; error bars represent standard deviation.

FIG. 11A-FIG. 11K show that SSX preferentially binds H2A K119Ub-marked nucleosomes to promote BAF complex targeting to polycomb-repressed loci. FIG. 11A shows CERES dependency scores (fitness dropout) derived from genome-scale fitness screens performed using CRISPR-Cas9-based methods (Achilles, Broad Institute; available on the World Wide Web at depmap.org/portal/achilles/). Difference is the score calculated between SYO1 (SS18-SSX+) cells and SW982 cells (negative for fusion, histologic mimic). mSWI/SNF, PRC1, PRC2 members are shown. FIG. 11B shows SS18 localization (by ChIP-seq) in SYO-1 cells treated with either scrambled KD or shSS18-SSX, aligned with H2AUb119 ChIP-seq in the scrambled KD condition. FIG. 11C shows example tracks at the SLIT3 locus reflecting co-localization of SS18-SSX BAF complexes, H2AUb, and RING1B (PRC1). FIG. 11D shows GST-SSX pull down experiments performed using either WT nucleosomes or H2A K119Ub nucleosomes. H3 immunoblot is used for assessment of nucleosome binding to GST-SSX. FIG. 11E shows alphalisa experiment performed with GST-SSX and 10 nM biotinylated nucleosomes of either WT, H2AUb or H2BUb nucleosomes. EC50 measurements are shown. n=3 experiments. FIG. 11F shows pull down experiments using endogenous, fully-assembled HA-SS18- or HA-SS18-SSX-bound BAF complexes incubated with either WT nucleosomes (unmodified) or H2A K119Ub-modified nucleosomes. SMARCA4 and H3 immunoblots are shown. FIG. 11G has two panels. The left panel shows the representation of PRC1 complex-nucleosome structure (McGinty et al. 2018; PDB: 4R8P), indicating regions mutagenized. The right panel shows the immunoblot of representative mutations which inhibit H2A K119Ub deposition absent changes to PRC1 structural integrity. FIG. 11H shows immunofluorescence imaging for RING1B (red), V5 SS18-SSX (green), with DAPI nuclear stain, and merged images in WT and RING1A/B dKO 293T cells with rescued conditions as indicated. FIG. 11I shows quantification of Barr body (inactive X Chr) localization for each condition, meanAU is plotted. Peptides were incubated −/+presence of USP2 treatment. Error bars=st.dev. FIG. 11J shows pull down experiments performed using either GST-SSX or GST-SSXdel7aa (acidic C-term DPEEDDE-->AAAAAAA (SEQ ID NOS 221-222, respectively, in order of appearance)) with WT nucleosomes or H2A K119Ub nucleosomes. H3 immunoblot is used for assessment of nucleosome binding to GST-SSX. FIG. 11K shows an alphalisa experiment performed with GST-SSX or GST-SSXdel7aa (acidic C-term) and 10 nM biotinylated nucleosomes of either WT or H2AUb nucleosomes. EC50 measurements are shown. n=3 experiments; error bars=st.dev. Data for GST-SSX with WT nucleosomes and H2A K119Ub nucleosomes are shared between FIG. 11E.

FIG. 12A-FIG. 12L show that SS18-SSX-bound BAF complexes preferentially bind H2A K119Ub-marked nucleosomes. FIG. 12A shows waterfall dependency plots for RING1B, PCGF3, PCGF5 and KDM2B genes across n=387 cell lines (Project DRIVE Dataset; available on the World Wide Web at oncologynibr.shinyapps.io/drive/; Novartis). SS cell lines containing the SS18-SSX fusion oncoprotein are highlighted in red. FIG. 12B shows H2A K119Ub and RING1B ChIP-seq tracks over selected loci, aligned with SS18 (BAF) localization in SYO-1 cells treated with shScramble or shSS18-SSX. FIG. 12C shows MBP-SSX1 (78aa) pull down experiments which indicate capture of histones, and specifically, H2AUb species. FIG. 12D shows CERES dependency scores (fitness dropout) derived from genome-scale fitness screens performed using CRISPR-Cas9-based methods (Achilles, Broad Institute; available on the World Wide Web at depmap.org/portal/achilles/). Difference is the score calculated between SYO1, Yamato-SS, SCS241 (SS18-SSX+) cells and SW982 cells (negative for fusion, histologic mimic). Blue indicates enriched for dependency. mSWI/SNF, PRC1, PRC2 members are shown. FIG. 12E shows CERES and DEMETER Dependency scores for SSX1 and SS18 genes for CRISPR-Cas9 and RNAi datasets, respectively. Synovial sarcoma cell lines are indicated in pink; all other cell lines are represented in gray. FIG. 12F shows CERES and DEMETER Dependency scores for SSX1 and SS18 genes for CRISPR-Cas9 and RNAi datasets, respectively. Synovial sarcoma and soft tissue (SS cell lines) exhibit preferential dependency. (Project DRIVE; available on the World Wide Web at oncologynibr.shinyapps.io/drive/). SS cell lines containing the SS18-SSX fusion oncoprotein are highlighted in red. FIG. 12 G shows GST-SSX pull down experiments performed using either WT nucleosomes or H2A K119Ub nucleosomes. H3 immunoblot is used for assessment of nucleosome binding to GST-SSX. FIG. 12H shows streptavidin-based pull-down experiments using endogenous, fully-assembled HA-SS18- or HA-SS18-SSX-bound BAF complexes incubated with biotinylated WT nucleosomes (unmodified) or H2A K119Ub-modified nucleosomes. SMARCA4 and H3 immunoblots are shown. FIG. 12I shows that silver stain of the WT SS18 complexes and SS18-SSX fusion complexes isolated usin ammonium sulfate nuclear extraction protocol. Identified proteins labeled (Left). WB of the samples on the right indicating presence of histone H3 (Right). FIG. 12J shows pull down experiments performed using GST-SSX incubated with unmodified or a series of modified recombinant mononucleosomes, or endogenous mononucleosomes (mammalian, purified via MNase digestion from HEK-239T cells). FIG. 12K shows quantitative densitometry performed on experiment in FIG. 6D. FIG. 12L shows fluorescence polarization assays performed with fluorescently-labeled SSX1 (78aa) and either unmodified nucleosomes (blue curve) or H2A K119Ub-modified nucleosomes (red curve).

FIG. 13A-FIG. 13G show that SSX targeting requires PRC1 complex-mediated H2A K119Ub placement. FIG. 13A shows immunoblots performed on V5 IP and input protein levels in WT and RING1A/B double KO (dKO) HEK-293T cells. FIG. 13B shows an immunoblot of representative, structurally-guided RING1B mutations which inhibit H2AK119Ub deposition partially, fully, or not at all. FIG. 13C shows immunofluorescence imaging for RING1B (red), V5 SS18-SSX (green), with DAPI nuclear stain, and merged images. FIG. 13D shows peptide hybridization experiments. Representative images of SSX labeling of Barr bodies (inactive X) identified for each condition using H3K27me3 staining. Peptides (SSX or Scrambled) were incubated methanol-fixed cells, untreated or treated with USP2 deubiquitinating enzyme. FIG. 13E shows incubation of GST-SSX WT, SSX mutant variants, or UBQLN1-TUBE2 or hHR23A-TUBE1 (pos controls) with Ub-coated beads. FIG. 13F shows V5-SS18-SSX, H2A K119Ub, and H3K27me3 IF studies performed in WT and RING1A/B dKO 293T cells. FIG. 13G shows DMSO control or EZH2 inhibitor treatment (to inhibit H3K27me3 placement) indicates no change to SS18-SSX foci localized to Barr bodies.

FIG. 14A-FIG. 14B show a model for SS18-SSX-bound BAF complex nucleosome engagement. FIG. 14A shows a schematic of SS18 WT and the SS18-SSX fusion oncoprotein. FIG. 14B shows a model for BAF complex engagement on nucleosomes in WT and SS18-SSX fusion oncoprotein states. In WT complexes, the core module of BAF complexes engages the nucleosome acidic patch via the SMARCB1 C-terminal alpha helical domain (aa 351-385). Upon expression of SS18-SSX, the SSX alpha helical basic region (RLRERK (SEQ ID NO: 219)) dominantly engages the acidic patch, displacing SMARCB1, leading to its degradation, and changing the orientation of the BAF core module (Mashtalir et al. (2018) Cell 175:1272-1288) on the nucleosome. This SS18-SSX-specific conformation of BAF complexes exhibits strong preference for H2AUbK119-decorated nucleosomes, underpinning their preference for polycomb chromatin regions.

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

The present invention is based, at least in part, on the discovery of the mechanism by which the SS18-SSX oncogenic fusion protein engages with chromatin and directs BAF chromatin remodeling complexes to specialized target sites. Specifically, it was found that SSX contains a basic region that directly binds the nucleosome acidic patch, altering BAF complex subunit configuration and activity. Further, SSX-nucleosome binding is augmented by the presence of ubiquitylated H2A (H2A K119Ub) on nucleosomes, preferential recognition of which requires a second, conserved region of SSX. These dual reader-like features of SSX underlie the highly disease-specific, hallmark chromatin remodeling complex targeting, gene expression, and functional dependencies in synovial sarcoma. Collectively, these studies reveal a novel mechanism of chromatin localization with important biological and disease implications.

There is current no direct way to treat human synovial sarcoma, driven by the SS18-SSX fusion oncoprotein. A major reason behind this is that, until this invention, little is known about how SS18-SSX specifically engages with chromatin to “hijack” BAF chromatin remodeling complexes to new sites genome-wide to activate cancer-promoting gene expression. The present disclosure unveils an unexpected, direct interaction between SSX and the nucleosome, specifically, the acidic patch region of the nucleosome, and the preference for repressed heterochromatin marked by the H2A K119Ub mark. Thus, the present disclosure provides an accurate and biologically meaningful screening strategy to identify agents that break SS18-SSX or SS18-SSX-containing BAF complex- H2A K119Ub nucleosome contacts. Chemical matter revealed from such a screening is capable of treating and potentially curing this disease in a highly specific manner.

Accordingly, the present invention relates, in part, to methods and agents for treating synovial sarcoma by modulating the interaction between SS18-SSX oncogenic fusion protein and H2A K119Ub nucleosomes.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

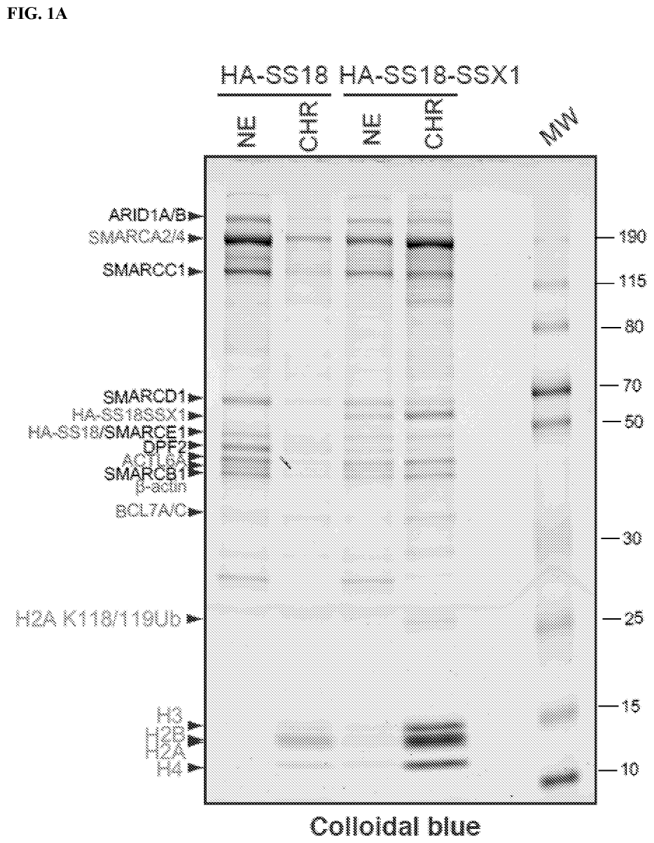

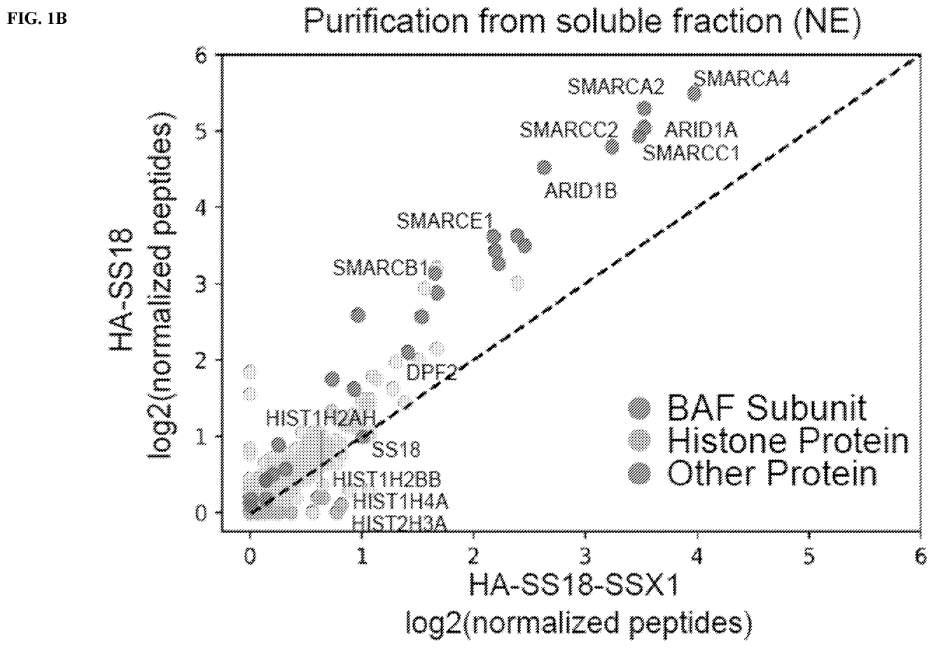

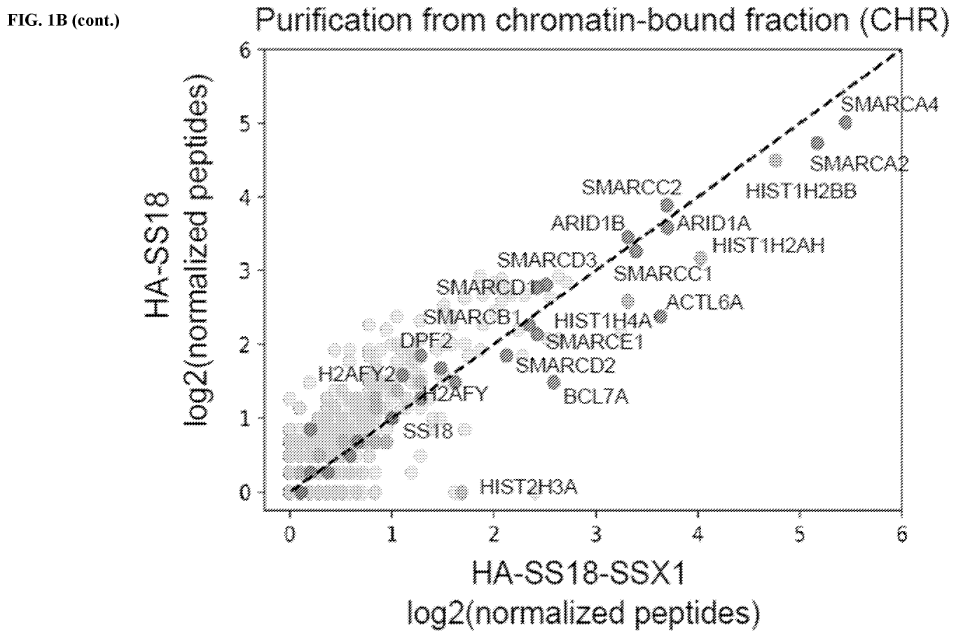

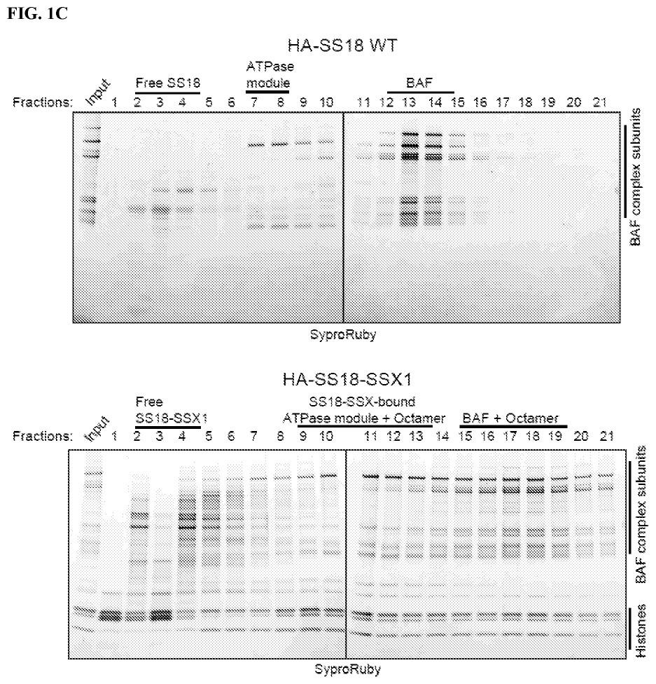

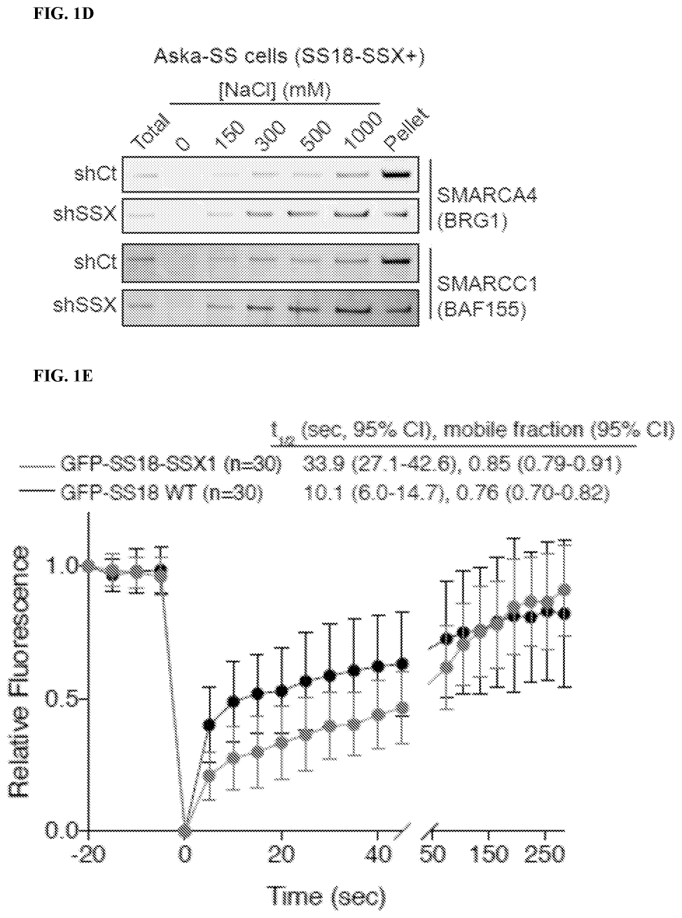

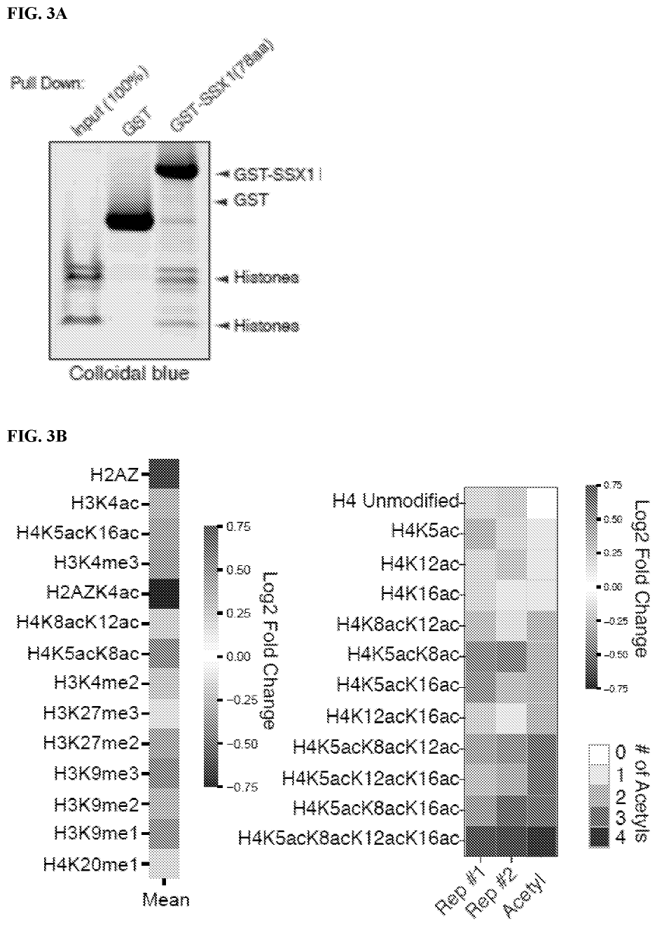

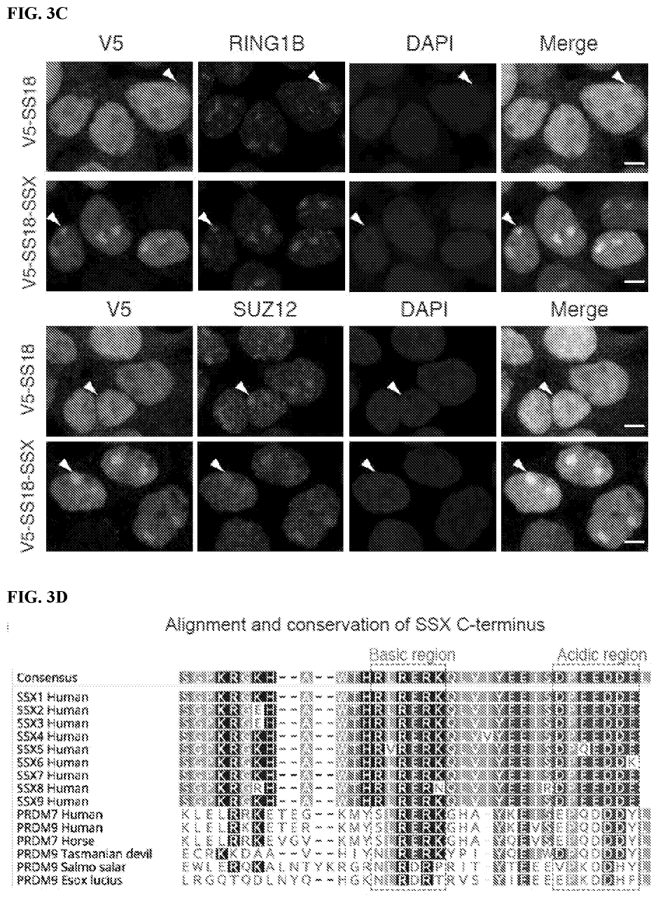

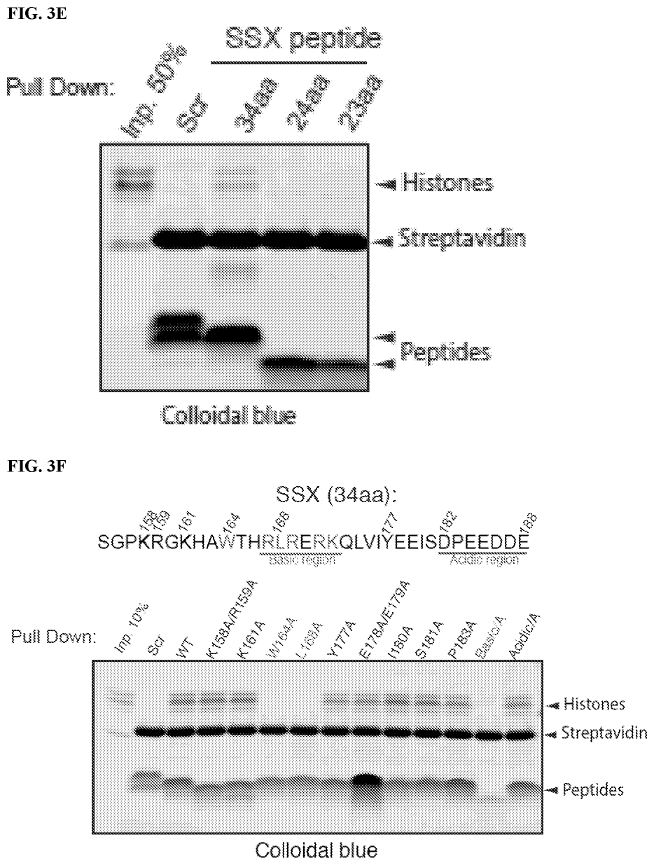

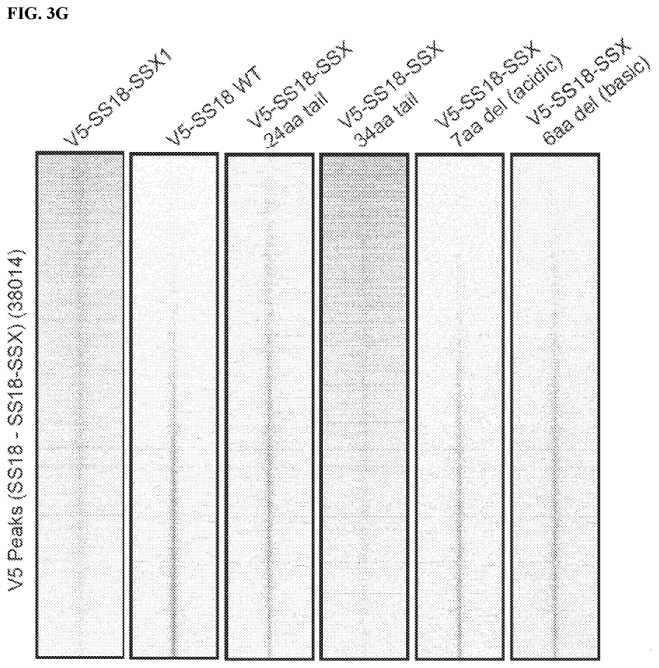

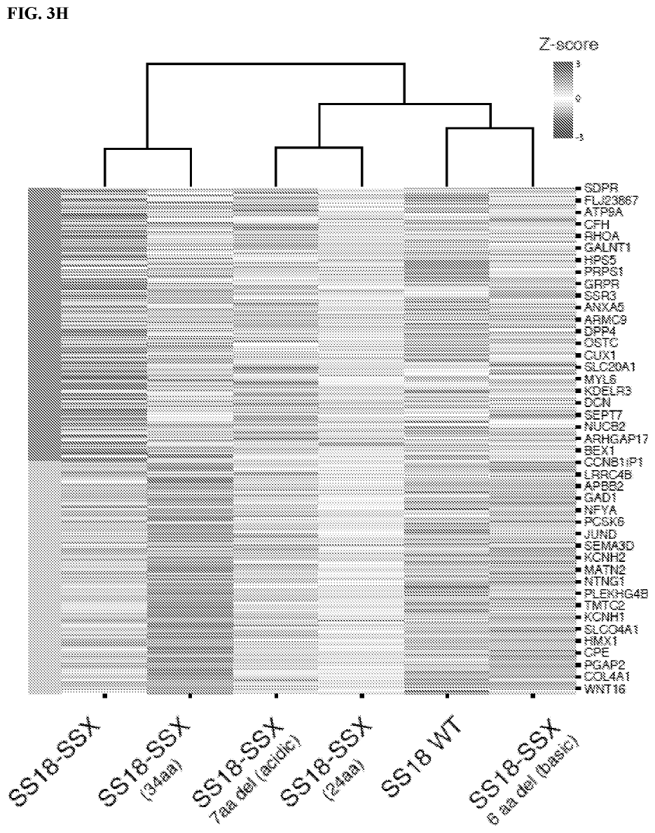

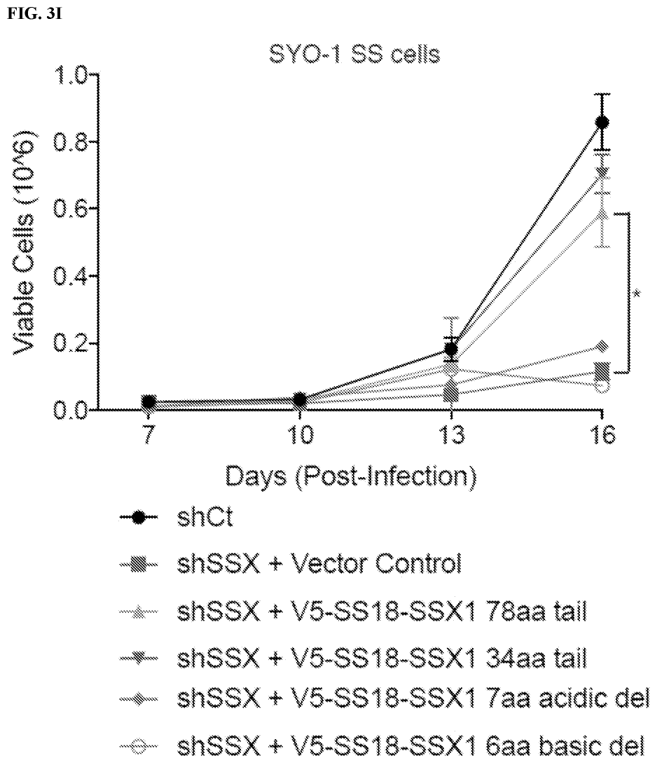

The term “SWI/SNF complex” refers to SWItch/Sucrose Non-Fermentable, a nucleosome remodeling complex found in both eukaryotes and prokaryotes (Neigeborn Carlson (1984) Genetics 108:845-858; Stern et al. (1984) J. Mol. Biol. 178:853-868). The SWI/SNF complex was first discovered in the yeast, Saccharomyces cerevisiae, named after yeast mating types switching (SWI) and sucrose nonfermenting (SNF) pathways (Workman and Kingston (1998) Annu Rev Biochem. 67:545-579; Sudarsanam and Winston (2000) Trends Genet. 16:345-351). It is a group of proteins comprising, at least, SWI1, SWI2/SNF2, SWI3, SWI5, and SWI6, as well as other polypeptides (Pazin and Kadonaga (1997) Cell 88:737-740). A genetic screening for suppressive mutations of the SWI/SNF phenotypes identified different histones and chromatin components, indicating that these proteins were possibly involved in histone binding and chromatin organization (Winston and Carlson (1992) Trends Genet. 8:387-391). Biochemical purification of the SWI/SNF2p in S. cerevisiae demonstrated that this protein was part of a complex containing an additional 11 polypeptides, with a combined molecular weight over 1.5 MDa. The SWI/SNF complex contains the ATPase Swi2/Snf2p, two actin-related proteins (Arp7p and Arp9) and other subunits involved in DNA and protein-protein interactions. The purified SWI/SNF complex was able to alter the nucleosome structure in an ATP-dependent manner (Workman and Kingston (1998), supra; Vignali et al. (2000) Mol Cell Biol. 20:1899-1910). The structures of the SWI/SNF and RSC complexes are highly conserved but not identical, reflecting an increasing complexity of chromatin (e.g., an increased genome size, the presence of DNA methylation, and more complex genetic organization) through evolution. For this reason, the SWI/SNF complex in higher eukaryotes maintains core components, but also substitute or add on other components with more specialized or tissue-specific domains. Yeast contains two distinct and similar remodeling complexes, SWI/SNF and RSC (Remodeling the Structure of Chromatin). In Drosophila, the two complexes are called BAP (Brahma Associated Protein) and PBAP (Polybromo-associated BAP) complexes. The human analogs are BAF (Brgl Associated Factors, or SWI/SNF-A) and PBAF (Polybromo-associated BAF, or SWI/SNF-B). The BAF complex comprises, at least, BAF250A (ARID1A), BAF250B (ARID1B), BAF57 (SMARCE1), BAF190/BRM (SMARCA2), BAF47 (SMARCB1), BAF53A (ACTL6A), BRG1/BAF190 (SMARCA4), BAF155 (SMARCC1), and BAF170 (SMARCC2). The PBAF complex comprises, at last, BAF200 (ARID2), BAF180 (PBRM1), BRD7, BAF45A (PHF10), BRG1/BAF190 (SMARCA4), BAF155 (SMARCC1), and BAF170 (SMARCC2). As in Drosophila, human BAF and PBAF share the different core components BAF47, BAF57, BAF60, BAF155, BAF170, BAF45 and the two actins b-Actin and BAF53 (Mohrmann and Verrijzer (2005) Biochim Biophys Acta. 1681:59-73). The central core of the BAF and PBAF is the ATPase catalytic subunit BRG1/hBRM, which contains multiple domains to bind to other protein subunits and acetylated histones. For a summary of different complex subunits and their domain structure, see Tang et al. (2010) Prog Biophys Mol Biol. 102:122-128 (e.g., FIG. 3), Hohmann and Vakoc (2014) Trends Genet. 30:356-363 (e.g., FIG. 1), and Kadoch and Crabtree (2015) Sci. Adv. 1:e1500447. For chromatin remodeling, the SWI/SNF complex use the energy of ATP hydrolysis to slide the DNA around the nucleosome. The first step consists in the binding between the remodeler and the nucleosome. This binding occurs with nanomolar affinity and reduces the digestion of nucleosomal DNA by nucleases. The 3-D structure of the yeast RSC complex was first solved and imaged using negative stain electron microscopy (Asturias et al. (2002) Proc Natl Acad Sci USA 99:13477-13480). The first Cryo-EM structure of the yeast SWI/SNF complex was published in 2008 (Dechassa et al. 2008). DNA footprinting data showed that the SWI/SNF complex makes close contacts with only one gyre of nucleosomal DNA. Protein crosslinking showed that the ATPase SWI2/SNF2p and Swi5p (the homologue of Ini1p in human), Snf6, Swi29, Snf11 and Sw82p (not conserved in human) make close contact with the histones. Several individual SWI/SNF subunits are encoded by gene families, whose protein products are mutually exclusive in the complex (Wu et al. (2009) Cell 136:200-206). Thus, only one paralog is incorporated in a given SWI/SNF assembly. The only exceptions are BAF155 and BAF170, which are always present in the complex as homo- or hetero-dimers.

Combinatorial association of SWI/SNF subunits could in principle give rise to hundreds of distinct complexes, although the exact number has yet to be determined (Wu et al. (2009), supra). Genetic evidence indicates that distinct subunit configurations of SWI/SNF are equipped to perform specialized functions. As an example, SWI/SNF contains one of two ATPase subunits, BRG1 or BRM/SMARCA2, which share 75% amino acid sequence identity (Khavari et al. (1993) Nature 366:170-174). While in certain cell types BRG1 and BRM can compensate for loss of the other subunit, in other contexts these two ATPases perform divergent functions (Strobeck et al. (2002) J Biol Chem. 277:4782-4789; Hoffman et al. (2014) Proc Natl Acad Sci USA. 111:3128-3133). In some cell types, BRG1 and BRM can even functionally oppose one another to regulate differentiation (Flowers et al. (2009) J Biol Chem. 284:10067-10075). The functional specificity of BRG1 and BRM has been linked to sequence variations near their N-terminus, which have different interaction specificities for transcription factors (Kadam and Emerson (2003) Mol Cell. 11:377-389). Another example of paralogous subunits that form mutually exclusive SWI/SNF complexes are ARID1A/BAF250A, ARID1B/BAF250B, and ARID2/BAF200. ARID1A and ARID1B share 60% sequence identity, but yet can perform opposing functions in regulating the cell cycle, with MYC being an important downstream target of each paralog (Nagl et al. (2007) EMBO J. 26:752-763). ARID2 has diverged considerably from ARID1A/ARID1B and exists in a unique SWI/SNF assembly known as PBAF (or SWI/SNF-B), which contains several unique subunits not found in ARID1A/B-containing complexes. The composition of SWI/SNF can also be dynamically reconfigured during cell fate transitions through cell type-specific expression patterns of certain subunits. For example, BAF53A/ACTL6A is repressed and replaced by BAF53B/ACTL6B during neuronal differentiation, a switch that is essential for proper neuronal functions in vivo (Lessard et al. (2007) Neuron 55:201-215). These studies stress that SWI/SNF in fact represents a collection of multi-subunit complexes whose integrated functions control diverse cellular processes, which is also incorporated in the scope of definitions of the instant disclosure. Two recently published meta-analyses of cancer genome sequencing data estimate that nearly 20% of human cancers harbor mutations in one (or more) of the genes encoding SWI/SNF (Kadoch et al. (2013) Nat Genet. 45:592-601; Shain and Pollack (2013) PLoS One. 8:e55119). Such mutations are generally loss-of-function, implicating SWI/SNF as a major tumor suppressor in diverse cancers. Specific SWI/SNF gene mutations are generally linked to a specific subset of cancer lineages: SNF5 is mutated in malignant rhabdoid tumors (MRT), PBRM1/BAF180 is frequently inactivated in renal carcinoma, and BRG1 is mutated in non-small cell lung cancer (NSCLC) and several other cancers. In the instant disclosure, the scope of “SWI/SNF complex” may cover at least one fraction or the whole complex (e.g., some or all subunit proteins/other components), either in the human BAF/PBAF forms or their homologs/orthologs in other species (e.g., the yeast and drosophila forms described herein). Preferably, a “SWI/SNF complex” described herein contains at least part of the full complex bio-functionality, such as binding to other subunits/components, binding to DNA/histone, catalyzing ATP, promoting chromatin remodeling, etc.

The term “BAF complex” refers to at least one type of mammalian SWI/SNF complexes. Its nucleosome remodeling activity can be reconstituted with a set of four core subunits (BRG1/SMARCA4, SNF5/SMARCB1, BAF155/SMARCC1, and BAF170/SMARCC2), which have orthologs in the yeast complex (Phelan et al. (1999) Mol Cell. 3:247-253). However, mammalian SWI/SNF contains several subunits not found in the yeast counterpart, which can provide interaction surfaces for chromatin (e.g. acetyl-lysine recognition by bromodomains) or transcription factors and thus contribute to the genomic targeting of the complex (Wang et al. (1996) EMBO J. 15:5370-5382; Wang et al. (1996) Genes Dev. 10:2117-2130; Nie et al. (2000)). A key attribute of mammalian SWI/SNF is the heterogeneity of subunit configurations that can exist in different tissues and even in a single cell type (e.g., as BAF, PBAF, neural progenitor BAF (npBAF), neuron BAF (nBAF), embryonic stem cell BAF (esBAF), etc.). In some embodiments, the BAF complex described herein refers to one type of mammalian SWI/SNF complexes, which is different from PBAF complexes.

The term “PBAF complex” refers to one type of mammalian SWI/SNF complexes originally known as SWI/SNF-B. It is highly related to the BAF complex and can be separated with conventional chromatographic approaches. For example, human BAF and PBAF complexes share multiple identical subunits (such as BRG, BAF170, BAF155, BAF60, BAF57, BAF53, BAF45, actin, SS18, and hSNF5/INI1). However, while BAF contains BAF250 subunit, PBAF contains BAF180 and BAF200, instead (Lemon et al. (2001) Nature 414:924-998; Yan et al. (2005) Genes Dev. 19:1662-1667). Moreover, they do have selectivity in regulating interferon-responsive genes (Yan et al. (2005), supra, showing that BAF200, but not BAF180, is required for PBAF to mediate expression of IFITMI gene induced by IFN-α, while the IFITM3 gene expression is dependent on BAF but not PBAF). Due to these differences, PBAF, but not BAF, was able to activate vitamin D receptor-dependent transcription on a chromatinzed template in vitro (Lemon et al. (2001), supra). The 3-D structure of human PBAF complex preserved in negative stain was found to be similar to yeast RSC but dramatically different from yeast SWI/SNF (Leschziner et al. (2005) Structure 13:267-275).

The term “BRG” or “BRG1/BAF190 (SMARCA4)” refers to a subunit of the SWI/SNF complex, which can be find in either BAF or PBAF complex. It is an ATP-depedendent helicase and a transcription activator, encoded by the SMARCA4 gene. BRG1 can also bind BRCA1, as well as regulate the expression of the tumorigenic protein CD44.

BRG1 is important for development past the pre-implantation stage. Without having a functional BRG1, exhibited with knockout research, the embryo will not hatch out of the zona pellucida, which will inhibit implantation from occurring on the endometrium (uterine wall). BRG1 is also crucial to the development of sperm. During the first stages of meiosis in spermatogenesis there are high levels of BRG1. When BRG1 is genetically damaged, meiosis is stopped in prophase 1, hindering the development of sperm and would result in infertility. More knockout research has concluded BRG1's aid in the development of smooth muscle. In a BRG1 knockout, smooth muscle in the gastrointestinal tract lacks contractility, and intestines are incomplete in some cases. Another defect occurring in knocking out BRG1 in smooth muscle development is heart complications such as an open ductus arteriosus after birth (Kim et al. (2012) Development 139:1133-1140; Zhang et al. (2011) Mol. Cell. Biol. 31:2618-2631). Mutations in SMARCA4 were first recognized in human lung cancer cell lines (Medina et al. (2008) Hum. Mut. 29:617-622). Later it was recognized that mutations exist in a significant frequency of medulloblastoma and pancreatic cancers among other tumor subtypes (Jones et al. (2012) Nature 488:100-105; Shain et al. (2012) Proc Natl Acad Sci USA 109:E252-E259; Shain and Pollack (2013), supra). Mutations in BRG1 (or SMARCA4) appear to be mutually exclusive with the presence of activation at any of the MYC-genes, which indicates that the BRG1 and MYC proteins are functionally related. Another recent study demonstrated a causal role of BRG1 in the control of retinoic acid and glucocorticoid-induced cell differentiation in lung cancer and in other tumor types. This enables the cancer cell to sustain undifferentiated gene expression programs that affect the control of key cellular processes. Furthermore, it explains why lung cancer and other solid tumors are completely refractory to treatments based on these compounds that are effective therapies for some types of leukemia (Romero et al. (2012) EMBO Mol. Med. 4:603-616). The role of BRG1 in sensitivity or resistance to anti-cancer drugs had been recently highlighted by the elucidation of the mechanisms of action of darinaparsin, an arsenic-based anti-cancer drugs. Darinaparsin has been shown to induce phosphorylation of BRG1, which leads to its exclusion from the chromatin. When excluded from the chromatin, BRG1 can no longer act as a transcriptional co-regulator.

This leads to the inability of cells to express HO-1, a cytoprotective enzyme. BRG1 has been shown to interact with proteins such as ACTL6A, ARID1A, ARID1B, BRCA1, CTNNB1, CBX5, CREBBP, CCNE1, ESR1, FANCA, HSP90B1, ING1, Myc, NR3C1, P53, POLR2A, PHB, SIN3A, SMARCB1, SMARCC1, SMARCC2, SMARCE1, STAT2, STK11, etc.

The term “BRG” or “BRG1/BAF190 (SMARCA4)” is intended to include fragments, variants (e.g., allelic variants), and derivatives thereof. Representative human BRG1(SMARCA4) cDNA and human BRG1 protein sequences are well-known in the art and are publicly available from the National Center for Biotechnology Information (NCBI). For example, seven different human BRG1 isoforms are known. Human BRG1 isoform A (NP_001122321.1) is encodable by the transcript variant 1 (NM_001128849.1), which is the longest transcript. Human BRG1 isoform B (NP_001122316.1 or NP_003063.2) is encodable by the transcript variant 2 (NM_001128844.1), which differs in the 5′ UTR and lacks an alternate exon in the 3′ coding region, compared to the variant 1, and also by the transcript variant 3 (NM_003072.3), which lacks an alternate exon in the 3′ coding region compared to variant 1. Human BRG1 isoform C (NP_001122317.1) is encodable by the transcript variant 4 (NM_001128845.1), which lacks two alternate in-frame exons and uses an alternate splice site in the 3′ coding region, compared to variant 1. Human BRG1 isoform D (NP_001122318.1) is encodable by the transcript variant 5 (NM_001128846.1), which lacks two alternate in-frame exons and uses two alternate splice sites in the 3′ coding region, compared to variant 1. Human BRG1 isoform E (NP_001122319.1) is encodable by the transcript variant 6 (NM_001128847.1), which lacks two alternate in-frame exons in the 3′ coding region, compared to variant 1. Human BRG1 isoform F (NP_001122320.1) is encodable by the transcript variant 7 (NM_001128848.1), which lacks two alternate in-frame exons and uses an alternate splice site in the 3′ coding region, compared to variant 1. Nucleic acid and polypeptide sequences of BRG1 orthologs in organisms other than humans are well known and include, for example, chimpanzee BRG1 (XM_016935029.1 and XP_016790518.1, XM_016935038.1 and XP_016790527.1, XM_016935039.1 and XP_016790528.1, XM_016935036.1 and XP_016790525.1, XM_016935037.1 and XP_016790526.1, XM_016935041.1 and XP_016790530.1, XM_016935040.1 and XP_016790529.1, XM_016935042.1 and XP_016790531.1, XM_016935043.1 and XP_016790532.1, XM_016935035.1 and XP_016790524.1, XM_016935032.1 and XP_016790521.1, XM_016935033.1 and XP_016790522.1, XM_016935030.1 and XP_016790519.1, XM_016935031.1 and XP_016790520.1, and XM_016935034.1 and XP_016790523.1), Rhesus monkey BRG1 (XM_015122901.1 and XP_014978387.1, XM_015122902.1 and XP_014978388.1, XM_015122903.1 and XP_014978389.1, XM_015122906.1 and XP_014978392.1, XM_015122905.1 and XP_014978391.1, XM_015122904.1 and XP_014978390.1, XM_015122907.1 and XP_014978393.1, XM_015122909.1 and XP_014978395.1, and XM_015122910.1 and XP_014978396.1), dog BRG1 (XM_014122046.1 and XP_013977521.1, XM_014122043.1 and XP_013977518.1, XM_014122042.1 and XP_013977517.1, XM_014122041.1 and XP_013977516.1, XM_014122045.1 and XP_013977520.1, and XM_014122044.1 and XP_013977519.1), cattle BRG1 (NM_001105614.1 and NP_001099084.1), rat BRG1 (NM_134368.1 and NP_599195.1).

Anti-BRG1 antibodies suitable for detecting BRG1 protein are well-known in the art and include, for example, MABE1118, MABE121, MABE60, and 07-478 (poly- and mono-clonal antibodies from EMD Millipore, Billerica, MA), AM26021PU-N, AP23972PU-N, TA322909, TA322910, TA327280, TA347049, TA347050, TA347851, and TA349038 (antibodies from OriGene Technologies, Rockville, MD), NB100-2594, AF5738, NBP2-22234, NBP2-41270, NBP1-51230, and NBP1-40379 (antibodes from Novus Biologicals, Littleton, CO), ab110641, ab4081, ab215998, ab108318, ab70558, ab118558, ab133257, ab92496, ab196535, and ab196315 (antibodies from AbCam, Cambridge, MA), Cat #: 720129, 730011, 730051, MA1-10062, PA5-17003, and PA5-17008 (antibodies from ThermoFisher Scientific, Waltham, MA), GTX633391, GTX32478, GTX31917, GTX16472, and GTX50842 (antibodies from GeneTex, Irvine, CA), antibody 7749 (ProSci, Poway, CA), Brg-1 (N-15), Brg-1 (N-15) X, Brg-1 (H-88), Brg-1 (H-88) X, Brg-1 (P-18), Brg-1 (P-18) X, Brg-1 (G-7), Brg-1 (G-7) X, Brg-1 (H-10), and Brg-1 (H-10) X (antibodies from Santa Cruz Biotechnology, Dallas, TX), antibody of Cat. AF5738 (R&D Systmes, Minneapolis, MN), etc. In addition, reagents are well-known for detecting BRG1 expression. Moreover, mutilple siRNA, shRNA, CRISPR constructs for reducing BRG1 Expression can be found in the commercial product lists of the above-referenced companies. PFI 3 is a known small molecule inhibitor of polybromo 1 and BRG1 (e.g., Cat. B7744 from APExBIO, Houston, TX). It is to be noted that the term can further be used to refer to any combination of features described herein regarding BRG1 molecules. For example, any combination of sequence composition, percentage identify, sequence length, domain structure, functional activity, etc. can be used to describe an BRG1 molecule of the present invention.

The term “BRM” or “BRM/BAF190 (SMARCA2)” refers to a subunit of the SWI/SNF complex, which can be found in either BAF or PBAF complexes. It is an ATP-dependent helicase and a transcription activator, encoded by the SMARCA2 gene. The catalytic core of the SWI/SNF complex can be either of two closely related ATPases, BRM or BRG1, with the potential that the choice of alternative subunits is a key determinant of specificity. Instead of impeding differentiation as was seen with BRG1 depletion, depletion of BRM caused accelerated progression to the differentiation phenotype. BRM was found to regulate genes different from those as BRG1 targets and be capable of overriding BRG1-dependent activation of the osteocalcin promoter, due to its interaction with different ARID family members (Flowers et al. (2009), supra). The known binding partners for BRM include, for example, ACTL6A, ARID1B, CEBPB, POLR2A, Prohibitin, SIN3A, SMARCB1, and SMARCC1.

The term “BRM” or “BRM/BAF190 (SMARCA2)” is intended to include fragments, variants (e.g., allelic variants), and derivatives thereof. Representative human BRM (SMARCA2) cDNA and human BRM protein sequences are well-known in the art and are publicly available from the National Center for Biotechnology Information (NCBI). For example, seven different human BRM isoforms are known. Human BRM isoform A (NP_003061.3 or NP_001276325.1) is encodable by the transcript variant 1 (NM_003070.4), which is the longest transcript, or the transcript variant 3 (NM_001289396.1), which differs in the 5′ UTR, compared to variant 1. Human BRM isoform B (NP_620614.2) is encodable by the transcript variant 2 (NM_139045.3), which lacks an alternate in-frame exon in the coding region, compared to variant 1. Human BRM isoform C (NP_001276326.1) is encodable by the transcript variant 4 (NM_001289397.1), which uses an alternate in-frame splice site and lacks an alternate in-frame exon in the 3′ coding region, compared to variant 1. Human BRM isoform D (NP_001276327.1) is encodable by the transcript variant 5 (NM_001289398.1), which differs in the 5′ UTR, lacks a portion of the 5′ coding region, and initiates translation at an alternate downstream start codon, compared to variant 1. Human BRM isoform E (NP_001276328.1) is encodable by the transcript variant 6 (NM_001289399.1), which differs in the 5′ UTR, lacks a portion of the 5′ coding region, and initiates translation at an alternate downstream start codon, compared to variant 1. Human BRM isoform F (NP_001276329.1) is encodable by the transcript variant 7 (NM_001289400.1), which differs in the 5′ UTR, lacks a portion of the 5′ coding region, and initiates translation at an alternate downstream start codon, compared to variant 1. Nucleic acid and polypeptide sequences of BRM orthologs in organisms other than humans are well known and include, for example, chimpanzee BRM (XM_016960529.1 and XP_016816018.1), dog BRG1 (XM_005615906.2 and XP_005615963.1, XM_845066.4 and XP_850159.1, XM_005615905.2 and XP_005615962.1, XM_005615904.2 and XP_005615961.1, XM_005615903.2 and XP_005615960.1, and XM_005615902.2 and XP_005615959.1), cattle BRM (NM_001099115.2 and NP_001092585.1), rat BRM (NM_001004446.1 and NP_001004446.1).

Anti-BRM antibodies suitable for detecting BRM protein are well-known in the art and include, for example, antibody MABE89 (EMD Millipore, Billerica, MA), antibody TA351725 (OriGene Technologies, Rockville, MD), NBP1-90015, NBP1-80042, NB100-55308, NB100-55309, NB100-55307, and H00006595-M06 (antibodes from Novus Biologicals, Littleton, CO), ab15597, ab12165, ab58188, and ab200480 (antibodies from AbCam, Cambridge, MA), Cat #: 11966 and 6889 (antibodies from Cell Signaling, Danvers, MA), etc. In addition, reagents are well-known for detecting BRM expression. Moreover, mutilple siRNA, shRNA, CRISPR constructs for reducing BRM Expression can be found in the commercial product lists of the above-referenced companies. For example, BRM RNAi product H00006595-R02 (Novus Biologicals), CRISPER gRNA products from GenScript, Piscataway, NJ, and other inhibitory RNA products from Origene, ViGene Biosciences (Rockville, MD), and Santa Cruz. It is to be noted that the term can further be used to refer to any combination of features described herein regarding BRM molecules. For example, any combination of sequence composition, percentage identify, sequence length, domain structure, functional activity, etc. can be used to describe an BRM molecule of the present invention.

The term “BAF250A” or “ARID1A” refers to AT-rich interactive domain-containing protein 1A, a subunit of the SWI/SNF complex, which can be find in BAF but not PBAF complex. In humans there are two BAF250 isoforms, BAF250A/ARID1A and BAF250B/ARID1B. They are thought to be E3 ubiquitin ligases that target histone H2B (Li et al. (2010) Mol. Cell. Biol. 30:1673-1688). ARID1A is highly expressed in the spleen, thymus, prostate, testes, ovaries, small intestine, colon and peripheral leukocytes. ARID1A is involved in transcriptional activation and repression of select genes by chromatin remodeling. It is also involved in vitamin D-coupled transcription regulation by associating with the WINAC complex, a chromatin-remodeling complex recruited by vitamin D receptor. ARID1A belongs to the neural progenitors-specific chromatin remodeling (npBAF) and the neuron-specific chromatin remodeling (nBAF) complexes, which are involved in switching developing neurons from stem/progenitors to post-mitotic chromatin remodeling as they exit the cell cycle and become committed to their adult state. ARID1A also plays key roles in maintaining embryonic stem cell pluripotency and in cardiac development and function (Lei et al. (2012) J. Biol. Chem. 287:24255-24262; Gao et al. (2008) Proc. Natl. Acad. Sci. U.S.A. 105:6656-6661). Loss of BAF250a expression was seen in 42% of the ovarian clear cell carcinoma samples and 21% of the endometrioid carcinoma samples, compared with just 1% of the high-grade serous carcinoma samples. ARID1A deficiency also impairs the DNA damage checkpoint and sensitizes cells to PARP inhibitors (Shen et al. (2015) Cancer Discov. 5:752-767). Human ARID1A protein has 2285 amino acids and a molecular mass of 242045 Da, with at least a DNA-binding domain that can specifically bind an AT-rich DNA sequence, recognized by a SWI/SNF complex at the beta-globin locus, and a C-terminus domain for glucocorticoid receptor-dependent transcriptional activation. ARID1A has been shown to interact with proteins such as SMARCB1/BAF47 (Kato et al. (2002) J. Biol. Chem. 277:5498-505; Wang et al. (1996) EMBO J. 15:5370-5382) and SMARCA4/BRG1 (Wang et al. (1996), supra; Zhao et al. (1998) Cell 95:625-636), etc.

The term “BAF250A” or “ARID1A” is intended to include fragments, variants (e.g., allelic variants), and derivatives thereof. Representative human BAF250A (ARID1A) cDNA and human BAF250A (ARID1A) protein sequences are well-known in the art and are publicly available from the National Center for Biotechnology Information (NCBI). For example, two different human ARID1A isoforms are known. Human ARID1A isoform A (NP_006006.3) is encodable by the transcript variant 1 (NM_006015.4), which is the longer transcript. Human ARID1A isoform B (NP_624361.1) is encodable by the transcript variant 2 (NM_139135.2), which lacks a segment in the coding region compared to variant 1. Isoform B thus lacks an internal segment, compared to isoform A. Nucleic acid and polypeptide sequences of ARID1A orthologs in organisms other than humans are well known and include, for example, chimpanzee ARID1A (XM_016956953.1 and XP_016812442.1, XM_016956958.1 and XP_016812447.1, and XM_009451423.2 and XP_009449698.2), Rhesus monkey ARID1A (XM_015132119.1 and XP_014987605.1, and XM_015132127.1 and XP_014987613.1), dog ARID1A (XM_847453.5 and XP_852546.3, XM_005617743.2 and XP_005617800.1, XM_005617742.2 and XP_005617799.1, XM_005617744.2 and XP_005617801.1, XM_005617746.2 and XP_005617803.1, and XM_005617745.2 and XP_005617802.1), cattle ARID1A (NM_001205785.1 and NP_001192714.1), rat ARID1A (NM_001106635.1 and NP_001100105.1).

Anti-ARID1A antibodies suitable for detecting ARID1A protein are well-known in the art and include, for example, antibody Cat #04-080 (EMD Millipore, Billerica, MA), antibodies TA349170, TA350870, and TA350871 (OriGene Technologies, Rockville, MD), antibodies NBP1-88932, NB100-55334, NBP2-43566, NB100-55333, and H00008289-QO1 (Novus Biologicals, Littleton, CO), antibodies ab182560, ab182561, ab176395, and ab97995 (AbCam, Cambridge, MA), antibodies Cat #: 12354 and 12854 (Cell Signaling Technology, Danvers, MA), antibodies GTX129433, GTX129432, GTX632013, GTX12388, and GTX31619 (GeneTex, Irvine, CA), etc. In addition, reagents are well-known for detecting ARID1A expression. For example, multiple clinical tests for ARID1A are available at NIH Genetic Testing Registry (GTR®) (e.g., GTR Test ID: GTR000520952.1 for mental retardation, offered by Centogene AG, Germany). Moreover, multiple siRNA, shRNA, CRISPR constructs for reducing ARID1A Expression can be found in the commercial product lists of the above-referenced companies, such as RNAi products H00008289-RO1, H00008289-R02, and H00008289-R03 (Novus Biologicals) and CRISPR products KN301547G1 and KN301547G2 (Origene). Other CRISPR products include sc-400469 (Santa Cruz Biotechnology) and those from GenScript (Piscataway, NJ). It is to be noted that the term can further be used to refer to any combination of features described herein regarding ARID1A molecules. For example, any combination of sequence composition, percentage identify, sequence length, domain structure, functional activity, etc. can be used to describe an ARID1A molecule of the present invention.

The term “loss-of-function mutation” for BAF250A/ARID1A refers to any mutation in an ARID1A-related nucleic acid or protein that results in reduced or eliminated ARID1A protein amounts and/or function. For example, nucleic acid mutations include single-base substitutions, multi-base substitutions, insertion mutations, deletion mutations, frameshift mutations, missesnse mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. In some embodiments, the mutation is a “nonsynonymous mutation,” meaning that the mutation alters the amino acid sequence of ARID1A. Such mutations reduce or eliminate ARID1A protein amounts and/or function by eliminating proper coding sequences required for proper ARID1A protein translation and/or coding for ARID1A proteins that are non-functional or have reduced function (e.g., deletion of enzymatic and/or structural domains, reduction in protein stability, alteration of sub-cellular localization, and the like). Such mutations are well-known in the art. In addition, a representative list describing a wide variety of structural mutations correlated with the functional result of reduced or eliminated ARID1A protein amounts and/or function is described in the Tables and the Examples.

The term “BAF250B” or “ARID1B” refers to AT-rich interactive domain-containing protein 1B, a subunit of the SWI/SNF complex, which can be find in BAF but not PBAF complex. ARID1B and ARID1A are alternative and mutually exclusive ARID-subunits of the SWI/SNF complex. Germline mutations in ARID1B are associated with Coffin-Siris syndrome (Tsurusaki et al. (2012) Nat. Genet. 44:376-378; Santen et al. (2012) Nat. Genet. 44:379-380). Somatic mutations in ARID1B are associated with several cancer subtypes, indicating that it is a tumor suppressor gene (Shai and Pollack (2013) PLoS ONE 8:e55119; Sausen et al. (2013) Nat. Genet. 45:12-17; Shain et al. (2012) Proc. Natl. Acad. Sci. U.S.A. 109:E252-E259; Fujimoto et al. (2012) Nat. Genet. 44:760-764). Human ARID1A protein has 2236 amino acids and a molecular mass of 236123 Da, with at least a DNA-binding domain that can specifically bind an AT-rich DNA sequence, recognized by a SWI/SNF complex at the beta-globin locus, and a C-terminus domain for glucocorticoid receptor-dependent transcriptional activation. ARID1B has been shown to interact with SMARCA4/BRG1 (Hurlstone et al. (2002) Biochem. J. 364:255-264; Inoue et al. (2002) J. Biol. Chem. 277:41674-41685 and SMARCA2/BRM (Inoue et al. (2002), supra).

The term “BAF250B” or “ARID1B” is intended to include fragments, variants (e.g., allelic variants), and derivatives thereof. Representative human BAF250B (ARID1B) cDNA and human BAF250B (ARID1B) protein sequences are well-known in the art and are publicly available from the National Center for Biotechnology Information (NCBI). For example, three different human ARID1B isoforms are known. Human ARID1B isoform A (NP_059989.2) is encodable by the transcript variant 1 (NM_017519.2). Human ARID1B isoform B (NP_065783.3) is encodable by the transcript variant 2 (NM_020732.3). Human ARID1B isoform C (NP_001333742.1) is encodable by the transcript variant 3 (NM_001346813.1). Nucleic acid and polypeptide sequences of ARID1B orthologs in organisms other than humans are well known and include, for example, Rhesus monkey ARID1B (XM_015137088.1 and XP_014992574.1), dog ARID1B (XM_014112912.1 and XP_013968387.1), cattle ARID1B (XM_010808714.2 and XP_010807016.1, and XM_015464874.1 and XP_015320360.1), rat ARID1B (XM_017604567.1 and XP_017460056.1).

Anti-ARID1B antibodies suitable for detecting ARID1B protein are well-known in the art and include, for example, antibody Cat #ABE316 (EMD Millipore, Billerica, MA), antibody TA315663 (OriGene Technologies, Rockville, MD), antibodies H00057492-M02, H00057492-MO1, NB100-57485, NBP1-89358, and NB100-57484 (Novus Biologicals, Littleton, CO), antibodies ab57461, ab69571, ab84461, and ab163568 (AbCam, Cambridge, MA), antibodies Cat #: PA5-38739, PA5-49852, and PA5-50918 (ThermoFisher Scientific, Danvers, MA), antibodies GTX130708, GTX60275, and GTX56037 (GeneTex, Irvine, CA), ARID1B (KMN1) Antibody and other antibodies (Santa Cruz Biotechnology), etc. In addition, reagents are well-known for detecting ARID1B expression. For example, multiple clinical tests for ARID1B are available at NIH Genetic Testing Registry (GTR®) (e.g., GTR Test ID: GTR000520953.1 for mental retardation, offered by Centogene AG, Germany). Moreover, multiple siRNA, shRNA, CRISPR constructs for reducing ARID1B Expression can be found in the commercial product lists of the above-referenced companies, such as RNAi products H00057492-R03, H00057492-RO1, and H00057492-R02 (Novus Biologicals) and CRISPR products KN301548 and KN214830 (Origene). Other CRISPR products include sc-402365 (Santa Cruz Biotechnology) and those from GenScript (Piscataway, NJ). It is to be noted that the term can further be used to refer to any combination of features described herein regarding ARID1B molecules. For example, any combination of sequence composition, percentage identify, sequence length, domain structure, functional activity, etc. can be used to describe an ARID1B molecule of the present invention.

The term “loss-of-function mutation” for BAF250B/ARID1B refers to any mutation in an ARID1B-related nucleic acid or protein that results in reduced or eliminated ARID1B protein amounts and/or function. For example, nucleic acid mutations include single-base substitutions, multi-base substitutions, insertion mutations, deletion mutations, frameshift mutations, missesnse mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. In some embodiments, the mutation is a “nonsynonymous mutation,” meaning that the mutation alters the amino acid sequence of ARID1B. Such mutations reduce or eliminate ARID1B protein amounts and/or function by eliminating proper coding sequences required for proper ARID1B protein translation and/or coding for ARID1B proteins that are non-functional or have reduced function (e.g., deletion of enzymatic and/or structural domains, reduction in protein stability, alteration of sub-cellular localization, and the like). Such mutations are well-known in the art. In addition, a representative list describing a wide variety of structural mutations correlated with the functional result of reduced or eliminated ARID1B protein amounts and/or function is described in the Tables and the Examples.

The term “SMARCC1” refers to SWI/SNF related, matrix associated, actin dependent regulator of chromatin subfamily c member 1. SMARCC1 is a member of the SWI/SNF family of proteins, whose members display helicase and ATPase activities and which are thought to regulate transcription of certain genes by altering the chromatin structure around those genes. The encoded protein is part of the large ATP-dependent chromatin remodeling complex SNF/SWI and contains a predicted leucine zipper motif typical of many transcription factors. SMARCC1 is a component of SWI/SNF chromatin remodeling complexes that carry out key enzymatic activities, changing chromatin structure by altering DNA-histone contacts within a nucleosome in an ATP-dependent manner. SMARCC1 stimulates the ATPase activity of the catalytic subunit of the complex (Phelan et al. (1999) Mol Cell 3:247-253). SMARCC1 belongs to the neural progenitors-specific chromatin remodeling complex (npBAF complex) and the neuron-specific chromatin remodeling complex (nBAF complex). During neural development a switch from a stem/progenitor to a postmitotic chromatin remodeling mechanism occurs as neurons exit the cell cycle and become committed to their adult state. The transition from proliferating neural stem/progenitor cells to postmitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. Human SMARCC1 protein has 1105 amino acids and a molecular mass of 122867 Da. Binding partners of SMARCC1 include, e.g., NR3C1, SMARD1, TRIP12, CEBPB, KDM6B, and MKKS.

The term “SMARCC1” is intended to include fragments, variants (e.g., allelic variants), and derivatives thereof. Representative human SMARCC1 cDNA and human SMARCC1 protein sequences are well-known in the art and are publicly available from the National Center for Biotechnology Information (NCBI). For example, human SMARCC1 protein (NP_003065.3) is encodable by the transcript (NM_003074.3). Nucleic acid and polypeptide sequences of SMARCC1 orthologs in organisms other than humans are well known and include, for example, chimpanzee SMARCC1 (XM_016940956.2 and XP_016796445.1, XM_001154676.6 and XP_001154676.1, XM_016940957.1 and XP_016796446.1, and XM_009445383.3 and XP_009443658.1), Rhesus monkey SMARCC1 (XM_015126104.1 and XP_014981590.1, XM_015126103.1 and XP_014981589.1, XM_001083389.3 and XP_001083389.2, and XM_015126105.1 and XP_014981591.1), dog SMARCC1 (XM_533845.6 and XP_533845.2, XM_014122183.2 and XP_013977658.1, and XM_014122184.2 and XP_013977659.1), cattle SMARCC1 (XM_024983285.1 and XP_024839053.1), mouse SMARCC1 (NM_009211.2 and NP_033237.2), rat SMARCC1 (NM_001106861.1 and NP_001100331.1), chicken SMARCC1 (XM_025147375.1 and XP_025003143.1, and XM_015281170.2 and XP_015136656.2), tropical clawed frog SMARCC1 (XM_002942718.4 and XP_002942764.2), and zebrafish SMARCC1 (XM_003200246.5 and XP_003200294.1, and XM_005158282.4 and XP_005158339.1). Representative sequences of SMARCC1 orthologs are presented below in Table 1.

Anti-SMARCC1 antibodies suitable for detecting SMARCC1 protein are well-known in the art and include, for example, antibody TA334040 (Origene), antibodies NBP1-88720, NBP2-20415, NBP1-88721, and NB100-55312 (Novus Biologicals, Littleton, CO), antibodies ab172638, ab126180, and ab22355 (AbCam, Cambridge, MA), antibody Cat #PA5-30174 (ThermoFisher Scientific), antibody Cat #27-825 (ProSci, Poway, CA), etc. In addition, reagents are well-known for detecting SMARCC1. A clinical test of SMARCC1 for hereditary disese is available with the test ID no. GTR000558444.1 in NIH Genetic Testing Registry (GTR®), offered by Tempus Labs, Inc., (Chicago, IL). Moreover, mutilple siRNA, shRNA, CRISPR constructs for reducing SMARCC1 expression can be found in the commercial product lists of the above-referenced companies, such as siRNA products #sc-29780 and sc-29781 and CRISPR product #sc-400838 from Santa Cruz Biotechnology, RNAi products SR304474 and TL309245V, and CRISPR product KN208534 (Origene), and multiple CRISPR products from GenScript (Piscataway, NJ). It is to be noted that the term can further be used to refer to any combination of features described herein regarding SMARCC1 molecules. For example, any combination of sequence composition, percentage identify, sequence length, domain structure, functional activity, etc. can be used to describe a SMARCC1 molecule encompassed by the present invention.

The term “SMARCC2” refers to SWI/SNF related, matrix associated, actin dependent regulator of chromatin subfamily c member 2. SMARCC2 is an important paralog of gene SMARCC1. SMARCC2 is a member of the SWI/SNF family of proteins, whose members display helicase and ATPase activities and which are thought to regulate transcription of certain genes by altering the chromatin structure around those genes. The encoded protein is part of the large ATP-dependent chromatin remodeling complex SNF/SWI and contains a predicted leucine zipper motif typical of many transcription factors. SMARCC2 is a component of SWI/SNF chromatin remodeling complexes that carry out key enzymatic activities, changing chromatin structure by altering DNA-histone contacts within a nucleosome in an ATP-dependent manner (Kadam et al. (2000) Genes Dev 14:2441-2451). SMARCC2 can stimulate the ATPase activity of the catalytic subunit of the complex (Phelan et al. (1999) Mol Cell 3:247-253). SMARCC2 is required for CoREST dependent repression of neuronal specific gene promoters in non-neuronal cells (Battaglioli et al. (2002) J Biol Chem 277:41038-41045). SMARCC2 belongs to the neural progenitors-specific chromatin remodeling complex (npBAF complex) and the neuron-specific chromatin remodeling complex (nBAF complex). SMARCC2 is a critical regulator of myeloid differentiation, controlling granulocytopoiesis and the expression of genes involved in neutrophil granule formation. Human SMARCC2 protein has 1214 amino acids and a molecular mass of 132879 Da. Binding partners of SMARCC2 include, e.g., SIN3A, SMARD1, KDM6B, and RCOR1.

The term “SMARCC2” is intended to include fragments, variants (e.g., allelic variants), and derivatives thereof. Representative human SMARCC2 cDNA (NM_003074.3) and human SMARCC2 protein sequences (NP_003065.3) are well-known in the art and are publicly available from the National Center for Biotechnology Information (NCBI). For example, four different human SMARCC2 isoforms are known. Human SMARCC2 isoform a (NP_003066.2) is encodable by the transcript variant 1 (NM_003075.4). Human SMARCC2 isoform b (NP_620706.1) is encodable by the transcript variant 2 (NM_139067.3), which contains an alternate in-frame exon in the central coding region and uses an alternate in-frame splice site in the 3′ coding region, compared to variant 1. The encoded isoform (b), contains a novel internal segment, lacks a segment near the C-terminus, and is shorter than isoform a. Human SMARCC2 isoform c (NP_001123892.1) is encodable by the transcript variant 3 (NM_001130420.2), which contains an alternate in-frame exon in the central coding region and contains alternate in-frame segment in the 3′ coding region, compared to variant 1. The encoded isoform (c), contains a novel internal segment, lacks a segment near the C-terminus, and is shorter than isoform a. Human SMARCC2 isoform d (NP_001317217.1) is encodable by the transcript variant 4 (NM_001330288.1), which contains an alternate in-frame exon in the central coding region compared to variant 1. The encoded isoform (d), contains the same N- and C-termini, but is longer than isoform a. Nucleic acid and polypeptide sequences of SMARCC2 orthologs in organisms other than humans are well known and include, for example, chimpanzee SMARCC2 (XM_016923208.2 and XP_016778697.1, XM_016923212.2 and XP_016778701.1, XM_016923214.2 and XP_016778703.1, XM_016923210.2 and XP_016778699.1, XM_016923209.2 and XP_016778698.1, XM_016923213.2 and XP_016778702.1, XM_016923211.2 and XP_016778700.1, and XM_016923216.2 and XP_016778705.1), Rhesus monkey SMARCC2 (XM_015151975.1 and XP_015007461.1, XM_015151976.1 and XP_015007462.1, XM_015151974.1 and XP_015007460.1, XM_015151969.1 and XP_015007455.1, XM_015151972.1 and XP_015007458.1, XM_015151973.1 and XP_015007459.1, and XM_015151970.1 and XP_015007456.1), dog SMARCC2 (XM_022424046.1 and XP_022279754.1, XM_014117150.2 and XP_013972625.1, XM_014117149.2 and XP_013972624.1, XM_005625493.3 and XP_005625550.1, XM_014117151.2 and XP_013972626.1, XM_005625492.3 and XP_005625549.1, XM_005625495.3 and XP_005625552.1, XM_005625494.3 and XP_005625551.1, and XM_022424047.1 and XP_022279755.1), cattle SMARCC2 (NM_001172224.1 and NP_001165695.1), mouse SMARCC1 (NM_001114097.1 and NP_001107569.1, NM_001114096.1 and NP_001107568.1, and NM_198160.2 and NP_937803.1), rat SMARCC2 (XM_002729767.5 and XP_002729813.2, XM_006240805.3 and XP_006240867.1, XM_006240806.3 and XP_006240868.1, XM_001055795.6 and XP_001055795.1, XM_006240807.3 and XP_006240869.1, XM_008765050.2 and XP_008763272.1, XM_017595139.1 and XP_017450628.1, XM_001055673.6 and XP_001055673.1, and XM_001055738.6 and XP_001055738.1), and zebrafish SMARCC2 (XM_021474611.1 and XP_021330286.1). Representative sequences of SMARCC2 orthologs are presented below in Table 1.

Anti-SMARCC2 antibodies suitable for detecting SMARCC2 protein are well-known in the art and include, for example, antibody TA314552 (Origene), antibodies NBP1-90017 and NBP2-57277 (Novus Biologicals, Littleton, CO), antibodies ab71907, ab84453, and ab64853 (AbCam, Cambridge, MA), antibody Cat #PA5-54351 (ThermoFisher Scientific), etc. In addition, reagents are well-known for detecting SMARCC2. A clinical test of SMARCC2 for hereditary disese is available with the test ID no. GTR000546600.2 in NIH Genetic Testing Registry (GTR®), offered by Fulgent Clinical Diagnostics Lab (Temple City, CA). Moreover, mutilple siRNA, shRNA, CRISPR constructs for reducing SMARCC2 expression can be found in the commercial product lists of the above-referenced companies, such as siRNA products #sc-29782 and sc-29783 and CRISPR product #sc-402023 from Santa Cruz Biotechnology, RNAi products SR304475 and TL301505V, and CRISPR product KN203744 (Origene), and multiple CRISPR products from GenScript (Piscataway, NJ). It is to be noted that the term can further be used to refer to any combination of features described herein regarding SMARCC2 molecules. For example, any combination of sequence composition, percentage identify, sequence length, domain structure, functional activity, etc. can be used to describe a SMARCC2 molecule encompassed by the present invention.

The term “SMARCD1” refers to SWI/SNF related, matrix associated, actin dependent regulator of chromatin subfamily D member 1. SMARCD1 is a member of the SWI/SNF family of proteins, whose members display helicase and ATPase activities and which are thought to regulate transcription of certain genes by altering the chromatin structure around those genes. The encoded protein is part of the large ATP-dependent chromatin remodeling complex SNF/SWI and has sequence similarity to the yeast Swp73 protein. SMARCD1 is a component of SWI/SNF chromatin remodeling complexes that carry out key enzymatic activities, changing chromatin structure by altering DNA-histone contacts within a nucleosome in an ATP-dependent manner (Wang et al. (1996) Genes Dev 10:2117-2130). SMARCD1 belongs to the neural progenitors-specific chromatin remodeling complex (npBAF complex) and the neuron-specific chromatin remodeling complex (nBAF complex). SMARCD1 has a strong influence on vitamin D-mediated transcriptional activity from an enhancer vitamin D receptor element (VDRE). SMARCD1 a link between mammalian SWI-SNF-like chromatin remodeling complexes and the vitamin D receptor (VDR) heterodimer (Koszewski et al. (2003) J Steroid Biochem Mol Biol 87:223-231). SMARCD1 mediates critical interactions between nuclear receptors and the BRG1/SMARCA4 chromatin-remodeling complex for transactivation (Hsiao et al. (2003) Mol Cell Biol 23:6210-6220). Human SMARCD1 protein has 515 amino acids and a molecular mass of 58233 Da. Binding partners of SMARCD1 include, e.g., ESR1, NR3C1, NR1H4, PGR, SMARCA4, SMARCC1 and SMARCC2.

The term “SMARCD1” is intended to include fragments, variants (e.g., allelic variants), and derivatives thereof. Representative human SMARCD1 cDNA and human SMARCD1 protein sequences are well-known in the art and are publicly available from the National Center for Biotechnology Information (NCBI). For example, two different human SMARCD1 isoforms are known. Human SMARCD1 isoform a (NP_003067.3) is encodable by the transcript variant 1 (NM_003076.4), which is the longer transcript.

Human SMARCD1 isoform b (NP_620710.2) is encodable by the transcript variant 2 (NM_139071.2), which lacks an alternate in-frame exon, compared to variant 1, resulting in a shorter protein (isoform b), compared to isoform a. Nucleic acid and polypeptide sequences of SMARCD1 orthologs in organisms other than humans are well known and include, for example, chimpanzee SMARCD1 (XM_016923432.2 and XP_016778921.1, XM_016923431.2 and XP_016778920.1, and XM_016923433.2 and XP_016778922.1), Rhesus monkey SMARCD1 (XM_001111275.3 and XP_001111275.3, XM_001111166.3 and XP_001111166.3, and XM_001111207.3 and XP_001111207.3), dog SMARCD1 (XM_543674.6 and XP_543674.4), cattle SMARCD1 (NM_001038559.2 and NP_001033648.1), mouse SMARCD1 (NM_031842.2 and NP_114030.2), rat SMARCD1 (NM_001108752.1 and NP_001102222.1), chicken SMARCD1 (XM_424488.6 and XP_424488.3), tropical clawed frog SMARCD1 (NM_001004862.1 and NP_001004862.1), and zebrafish SMARCD1 (NM_198358.1 and NP_938172.1). Representative sequences of SMARCD1 orthologs are presented below in Table 1.

Anti-SMARCD1 antibodies suitable for detecting SMARCD1 protein are well-known in the art and include, for example, antibody TA344378 (Origene), antibodies NBP1-88719 and NBP2-20417 (Novus Biologicals, Littleton, CO), antibodies ab224229, ab83208, and ab86029 (AbCam, Cambridge, MA), antibody Cat #PA5-52049 (ThermoFisher Scientific), etc. In addition, reagents are well-known for detecting SMARCD1. A clinical test of SMARCD1 for hereditary disese is available with the test ID no. GTR000558444.1 in NIH Genetic Testing Registry (GTR®), offered by Tempus Labs, Inc., (Chicago, IL). Moreover, mutilple siRNA, shRNA, CRISPR constructs for reducing SMARCD1 expression can be found in the commercial product lists of the above-referenced companies, such as siRNA products #sc-72597 and sc-725983 and CRISPR product #sc-402641 from Santa Cruz Biotechnology, RNAi products SR304476 and TL301504V, and CRISPR product KN203474 (Origene), and multiple CRISPR products from GenScript (Piscataway, NJ). It is to be noted that the term can further be used to refer to any combination of features described herein regarding SMARCD1 molecules. For example, any combination of sequence composition, percentage identify, sequence length, domain structure, functional activity, etc. can be used to describe a SMARCD1 molecule encompassed by the present invention.

The term “SMARCD2” refers to SWI/SNF related, matrix associated, actin dependent regulator of chromatin subfamily D member 2. SMARCD2 is a member of the SWI/SNF family of proteins, whose members display helicase and ATPase activities and which are thought to regulate transcription of certain genes by altering the chromatin structure around those genes. The encoded protein is part of the large ATP-dependent chromatin remodeling complex SNF/SWI and has sequence similarity to the yeast Swp73 protein. SMARCD2 is a component of SWI/SNF chromatin remodeling complexes that carry out key enzymatic activities, changing chromatin structure by altering DNA-histone contacts within a nucleosome in an ATP-dependent manner (Euskirchen et al. (2012) J Biol Chem 287:30897-30905; Kadoch et al. (2015) Sci Adv 1(5):e1500447). SMARCD2 is a critical regulator of myeloid differentiation, controlling granulocytopoiesis and the expression of genes involved in neutrophil granule formation (Witzel et al. (2017) Nat Genet 49:742-752). Human SMARCD2 protein has 531 amino acids and a molecular mass of 589213 Da. Binding partners of SMARCD2 include, e.g., UNKL and CEBPE.

The term “SMARCD2” is intended to include fragments, variants (e.g., allelic variants), and derivatives thereof. Representative human SMARCD2 cDNA and human SMARCD2 protein sequences are well-known in the art and are publicly available from the National Center for Biotechnology Information (NCBI). For example, three different human SMARCD2 isoforms are known. Human SMARCD2 isoform 1 (NP_001091896.1) is encodable by the transcript variant 1 (NM_001098426.1). Human SMARCD2 isoform 2 (NP_001317368.1) is encodable by the transcript variant 2 (NM_001330439.1). Human SMARCD2 isoform 3 (NP_001317369.1) is encodable by the transcript variant 3 (NM_001330440.1). Nucleic acid and polypeptide sequences of SMARCD2 orthologs in organisms other than humans are well known and include, for example, chimpanzee SMARCD2 (XM_009433047.3 and XP_009431322.1, XM_001148723.6 and XP_001148723.1, XM_009433048.3 and XP_009431323.1, XM_009433049.3 and XP_009431324.1, XM_024350546.1 and XP_024206314.1, and XM_024350547.1 and XP_024206315.1), Rhesus monkey SMARCD2 (XM_015120093.1 and XP_014975579.1), dog SMARCD2 (XM_022422831.1 and XP_022278539.1, XM_005624251.3 and XP_005624308.1, XM_845276.5 and XP_850369.1, and XM_005624252.3 and XP_005624309.1), cattle SMARCD2 (NM_001205462.3 and NP_001192391.1), mouse SMARCC1 (NM_001130187.1 and NP_001123659.1, and NM_031878.2 and NP_114084.2), rat SMARCD2 (NM_031983.2 and NP_114189.1), chicken SMARCD2 (XM_015299406.2 and XP_015154892.1), tropical clawed frog SMARCD2 (NM_001045802.1 and NP_001039267.1), and zebrafish SMARCD2 (XM_687657.6 and XP_692749.2, and XM_021480266.1 and XP_021335941.1). Representative sequences of SMARCD2 orthologs are presented below in Table 1.

Anti-SMARCD2 antibodies suitable for detecting SMARCD2 protein are well-known in the art and include, for example, antibody TA335791 (Origene), antibodies H00006603-M02 and H00006603-MO1 (Novus Biologicals, Littleton, CO), antibodies ab81622, ab56241, and ab221084 (AbCam, Cambridge, MA), antibody Cat #51-805 (ProSci, Poway, CA), etc. In addition, reagents are well-known for detecting SMARCD2. A clinical test of SMARCD2 for hereditary disese is available with the test ID no. GTR000558444.1 in NIH Genetic Testing Registry (GTR®), offered by Tempus Labs, Inc., (Chicago, IL). Moreover, mutilple siRNA, shRNA, CRISPR constructs for reducing SMARCD2 expression can be found in the commercial product lists of the above-referenced companies, such as siRNA products #sc-93762 and sc-153618 and CRISPR product #sc-403091 from Santa Cruz Biotechnology, RNAi products SR304477 and TL309244V, and CRISPR product KN214286 (Origene), and multiple CRISPR products from GenScript (Piscataway, NJ). It is to be noted that the term can further be used to refer to any combination of features described herein regarding SMARCD2 molecules. For example, any combination of sequence composition, percentage identify, sequence length, domain structure, functional activity, etc. can be used to describe a SMARCD2 molecule encompassed by the present invention.

The term “SMARCD3” refers to SWI/SNF related, matrix associated, actin dependent regulator of chromatin subfamily D member 3. SMARCD3 is a member of the SWI/SNF family of proteins, whose members display helicase and ATPase activities and which are thought to regulate transcription of certain genes by altering the chromatin structure around those genes. The encoded protein is part of the large ATP-dependent chromatin remodeling complex SNF/SWI and has sequence similarity to the yeast Swp73 protein. SMARCD3 is a component of SWI/SNF chromatin remodeling complexes that carry out key enzymatic activities, changing chromatin structure by altering DNA-histone contacts within a nucleosome in an ATP-dependent manner. SMARCD3 stimulates nuclear receptor mediated transcription. SMARCD3 belongs to the neural progenitors-specific chromatin remodeling complex (npBAF complex) and the neuron-specific chromatin remodeling complex (nBAF complex). Human SMARCD3 protein has 483 amino acids and a molecular mass of 55016 Da. Binding partners of SMARCD3 include, e.g., PPARG/NR1C3, RXRA/NR1F1, ESR1, NR5A1, NR5A2/LRH1 and other transcriptional activators including the HLH protein SREBF1/SREBP1 and the homeobox protein PBX1.

The term “SMARCD3” is intended to include fragments, variants (e.g., allelic variants), and derivatives thereof. Representative human SMARCD3 cDNA and human SMARCD3 protein sequences are well-known in the art and are publicly available from the National Center for Biotechnology Information (NCBI). For example, two different human SMARCD3 isoforms are known. Human SMARCD3 isoform 1 (NP_001003802.1 and NP_003069.2) is encodable by the transcript variant 1 (NM_001003802.1) and the transcript variant 2 (NM_003078.3). Human SMARCD2 isoform 2 (NP_001003801.1) is encodable by the transcript variant 3 (NM_001003801.1). Nucleic acid and polypeptide sequences of SMARCD3 orthologs in organisms other than humans are well known and include, for example, chimpanzee SMARCD3 (XM_016945944.2 and XP_016801433.1, XM_016945946.2 and XP_016801435.1, XM_016945945.2 and XP_016801434.1, and XM_016945943.2 and XP_016801432.1), Rhesus monkey SMARCD3 (NM_001260684.1 and NP_001247613.1), cattle SMARCD3 (NM_001078154.1 and NP_001071622.1), mouse SMARCC3 (NM_025891.3 and NP_080167.3), rat SMARCD3 (NM_001011966.1 and NP_001011966.1). Representative sequences of SMARCD3 orthologs are presented below in Table 1.

Anti-SMARCD3 antibodies suitable for detecting SMARCD3 protein are well-known in the art and include, for example, antibody TA811107 (Origene), antibodies H00006604-MO1 and NBP2-39013 (Novus Biologicals, Littleton, CO), antibodies ab171075, ab131326, and ab50556 (AbCam, Cambridge, MA), antibody Cat #720131 (ThermoFisher Scientific), antibody Cat #28-327 (ProSci, Poway, CA), etc. In addition, reagents are well-known for detecting SMARCD3. A clinical test of SMARCD3 for hereditary disese is available with the test ID no. GTR000558444.1 in NIH Genetic Testing Registry (GTR®), offered by Tempus Labs, Inc., (Chicago, IL). Moreover, mutilple siRNA, shRNA, CRISPR constructs for reducing SMARCD3 expression can be found in the commercial product lists of the above-referenced companies, such as siRNA products #sc-89355 and sc-108054 and CRISPR product #sc-402705 from Santa Cruz Biotechnology, RNAi products SR304478 and TL309243V, and CRISPR product KN201135 (Origene), and multiple CRISPR products from GenScript (Piscataway, NJ). It is to be noted that the term can further be used to refer to any combination of features described herein regarding SMARCD3 molecules. For example, any combination of sequence composition, percentage identify, sequence length, domain structure, functional activity, etc. can be used to describe a SMARCD3 molecule encompassed by the present invention.

The term “SMARCE1” refers to SWI/SNF related, matrix associated, actin dependent regulator of chromatin subfamily E member 1. The protein encoded by this gene is part of the large ATP-dependent chromatin remodeling complex SWI/SNF, which is required for transcriptional activation of genes normally repressed by chromatin. The encoded protein, either alone or when in the SWI/SNF complex, can bind to 4-way junction DNA, which is thought to mimic the topology of DNA as it enters or exits the nucleosome. The protein contains a DNA-binding HMG domain, but disruption of this domain does not abolish the DNA-binding or nucleosome-displacement activities of the SWI/SNF complex. Unlike most of the SWI/SNF complex proteins, this protein has no yeast counterpart. SMARCE1 is a component of SWI/SNF chromatin remodeling complexes that carry out key enzymatic activities, changing chromatin structure by altering DNA-histone contacts within a nucleosome in an ATP-dependent manner. SMARCE1 belongs to the neural progenitors-specific chromatin remodeling complex (npBAF complex) and the neuron-specific chromatin remodeling complex (nBAF complex). SMARCE1 is required for the coactivation of estrogen responsive promoters by SWI/SNF complexes and the SRC/p160 family of histone acetyltransferases (HATs). SMARCE1 also specifically interacts with the CoREST corepressor resulting in repression of neuronal specific gene promoters in non-neuronal cells. Human SMARCE1 protein has 411 amino acids and a molecular mass of 46649 Da. SMARCE1 interacts with BRDT, and also binds to the SRC/p160 family of histone acetyltransferases (HATs) composed of NCOA1, NCOA2, and NCOA3. SMARCE1 interacts with RCOR1/CoREST, NR3C1 and ZMIM2/ZIMP7.

The term “SMARCE1” is intended to include fragments, variants (e.g., allelic variants), and derivatives thereof. Representative human SMARCE1 cDNA and human SMARCE1 protein sequences are well-known in the art and are publicly available from the National Center for Biotechnology Information (NCBI). For example, human SMARCE1 protein (NP_003070.3) is encodable by transcript (NM_003079.4). Nucleic acid and polypeptide sequences of SMARCE1 orthologs in organisms other than humans are well known and include, for example, chimpanzee SMARCE1 (XM_009432223.3 and XP_009430498.1, XM_511478.7 and XP_511478.2, XM_009432222.3 and XP_009430497.1, and XM_001169953.6 and XP_001169953.1), Rhesus monkey SMARCE1 (NM_001261306.1 and NP_001248235.1), cattle SMARCE1 (NM_001099116.2 and NP_001092586.1), mouse SMARCE1 (NM_020618.4 and NP_065643.1), rat SMARCE1 (NM_001024993.1 and NP_001020164.1), chicken SMARCE1 (NM_001006335.2 and NP_001006335.2), tropical clawed frog SMARCE1 (NM_001005436.1 and NP_001005436.1), and zebrafish SMARCE1 (NM_201298.1 and NP_958455.2). Representative sequences of SMARCE1 orthologs are presented below in Table 1.

Anti-SMARCE1 antibodies suitable for detecting SMARCE1 protein are well-known in the art and include, for example, antibody TA335790 (Origene), antibodies NBP1-90012 and NB100-2591 (Novus Biologicals, Littleton, CO), antibodies ab131328, ab228750, and ab137081 (AbCam, Cambridge, MA), antibody Cat #PA5-18185 (ThermoFisher Scientific), antibody Cat #57-670 (ProSci, Poway, CA), etc. In addition, reagents are well-known for detecting SMARCE1. A clinical test of SMARCE1 for hereditary disese is available with the test ID no. GTR000558444.1 in NIH Genetic Testing Registry (GTR®), offered by Tempus Labs, Inc., (Chicago, IL). Moreover, mutilple siRNA, shRNA, CRISPR constructs for reducing SMARCE1 expression can be found in the commercial product lists of the above-referenced companies, such as siRNA products #sc-45940 and sc-45941 and CRISPR product #sc-404713 from Santa Cruz Biotechnology, RNAi products SR304479 and TL309242, and CRISPR product KN217885 (Origene), and multiple CRISPR products from GenScript (Piscataway, NJ). It is to be noted that the term can further be used to refer to any combination of features described herein regarding SMARCE1 molecules. For example, any combination of sequence composition, percentage identify, sequence length, domain structure, functional activity, etc. can be used to describe a SMARCE1 molecule encompassed by the present invention.

The term “DPF1” refers to Double PHD Fingers 1. DPF1 has an important role in developing neurons by participating in regulation of cell survival, possibly as a neurospecific transcription factor. DPF1 belongs to the neuron-specific chromatin remodeling complex (nBAF complex). During neural development a switch from a stem/progenitor to a post-mitotic chromatin remodeling mechanism occurs as neurons exit the cell cycle and become committed to their adult state. The transition from proliferating neural stem/progenitor cells to post-mitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. Human DPF1 protein has 380 amino acids and a molecular mass of 425029 Da. DPF1 is a component of neuron-specific chromatin remodeling complex (nBAF complex) composed of at least, ARID1A/BAF250A or ARID1B/BAF250B, SMARCD1/BAF60A, SMARCD3/BAF60C, SMARCA2/BRM/BAF190B, SMARCA4/BRG1/BAF190A, SMARCB1/BAF47, SMARCC1/BAF155, SMARCE1/BAF57, SMARCC2/BAF170, DPF1/BAF45B, DPF3/BAF45C, ACTL6B/BAF53B and actin.

The term “DPF1” is intended to include fragments, variants (e.g., allelic variants), and derivatives thereof. Representative human DPF1 cDNA and human DPF1 protein sequences are well-known in the art and are publicly available from the National Center for Biotechnology Information (NCBI). For example, five different human DPF1 isoforms are known. Human DPF1 isoform a (NP_001128627.1) is encodable by the transcript variant 1 (NM_001135155.2). Human DPF1 isoform b (NP_004638.2) is encodable by the transcript variant 2 (NM_004647.3). Human DPF1 isoform c (NP_001128628.1) is encodable by the transcript variant 3 (NM_001135156.2). Human DPF1 isoform d (NP_001276907.1) is encodable by the transcript variant 4 (NM_001289978.1). Human DPF1 isoform e (NP_001350508.1) is encodable by the transcript variant 5 (NM_001363579.1). Nucleic acid and polypeptide sequences of DPF1 orthologs in organisms other than humans are well known and include, for example, Rhesus monkey DPF1 (XM_015123830.1 and XP_014979316.1, XM_015123829.1 and XP_014979315.1, XM_015123835.1 and XP_014979321.1, XM_015123831.1 and XP_014979317.1, XM_015123833.1 and XP_014979319.1, and XM_015123832.1 and XP_014979318.1), cattle DPF1 (NM_001076855.1 and NP_001070323.1), mouse DPF1 (NM_013874.2 and NP_038902.1), rat DPF1 (NM_001105729.3 and NP_001099199.2), and tropical clawed frog DPF1 (NM_001097276.1 and NP_001090745.1). Representative sequences of DPF1 orthologs are presented below in Table 1.

Anti-DPF1 antibodies suitable for detecting DPF1 protein are well-known in the art and include, for example, antibody TA311193 (Origene), antibodies NBP2-13932 and NBP2-19518 (Novus Biologicals, Littleton, CO), antibodies ab199299, ab173160, and ab3940 (AbCam, Cambridge, MA), antibody Cat #PA5-61895 (ThermoFisher Scientific), antibody Cat #28-079 (ProSci, Poway, CA), etc. In addition, reagents are well-known for detecting DPF1. Moreover, mutilple siRNA, shRNA, CRISPR constructs for reducing DPF1 expression can be found in the commercial product lists of the above-referenced companies, such as siRNA products #sc-97084 and sc-143155 and CRISPR product #sc-409539 from Santa Cruz Biotechnology, RNAi products SR305389 and TL313388V, and CRISPR product KN213721 (Origene), and multiple CRISPR products from GenScript (Piscataway, NJ). It is to be noted that the term can further be used to refer to any combination of features described herein regarding DPF1 molecules. For example, any combination of sequence composition, percentage identify, sequence length, domain structure, functional activity, etc. can be used to describe a DPF1 molecule encompassed by the present invention.

The term “DPF2” refers to Double PHD Fingers 2. DPF2 protein is a member of the d4 domain family, characterized by a zinc finger-like structural motif. It functions as a transcription factor which is necessary for the apoptotic response following deprivation of survival factors. It likely serves a regulatory role in rapid hematopoietic cell growth and turnover. This gene is considered a candidate gene for multiple endocrine neoplasia type I, an inherited cancer syndrome involving multiple parathyroid, enteropancreatic, and pituitary tumors. DPF2 is a transcription factor required for the apoptosis response following survival factor withdrawal from myeloid cells. DPF2 also has a role in the development and maturation of lymphoid cells. Human DPF2 protein has 391 amino acids and a molecular mass of 44155 Da.

The term “DPF2” is intended to include fragments, variants (e.g., allelic variants), and derivatives thereof. Representative human DPF2 cDNA and human DPF2 protein sequences are well-known in the art and are publicly available from the National Center for Biotechnology Information (NCBI). For example, two different human DPF2 isoforms are known. Human DPF2 isoform 1 (NP_006259.1) is encodable by the transcript variant 1 (NM_006268.4). Human DPF2 isoform 2 (NP_001317237.1) is encodable by the transcript variant 2 (NM_001330308.1). Nucleic acid and polypeptide sequences of DPF2 orthologs in organisms other than humans are well known and include, for example, chimpanzee DPF2 (NM_001246651.1 and NP_001233580.1), Rhesus monkey DPF2 (XM_002808062.2 and XP_002808108.2, and XM_015113800.1 and XP_014969286.1), dog DPF2 (XM_861495.5 and XP_866588.1, and XM_005631484.3 and XP_005631541.1), cattle DPF2 (NM_001100356.1 and NP_001093826.1), mouse DPF2 (NM_001291078.1 and NP_001278007.1, and NM_011262.5 and NP_035392.1), rat DPF2 (NM_001108516.1 and NP_001101986.1), chicken DPF2 (NM_204331.1 and NP_989662.1), tropical clawed frog DPF2 (NM_001197172.2 and NP_001184101.1), and zebrafish DPF2 (NM_001007152.1 and NP_001007153.1). Representative sequences of DPF2 orthologs are presented below in Table 1.

Anti-DPF2 antibodies suitable for detecting DPF2 protein are well-known in the art and include, for example, antibody TA312307 (Origene), antibodies NBP1-76512 and NBP1-87138 (Novus Biologicals, Littleton, CO), antibodies ab134942, ab232327, and ab227095 (AbCam, Cambridge, MA), etc. In addition, reagents are well-known for detecting DPF2. A clinical test of DPF2 for hereditary disese is available with the test ID no. GTR000536833.2 in NIH Genetic Testing Registry (GTR®), offered by Fulgent Genetics Clinical Diagnostics Lab (Temple City, CA). Moreover, mutilple siRNA, shRNA, CRISPR constructs for reducing DPF2 expression can be found in the commercial product lists of the above-referenced companies, such as siRNA products #sc-97031 and sc-143156 and CRISPR product #sc-404801-KO-2 from Santa Cruz Biotechnology, RNAi products SR304035 and TL313387V, and CRISPR product KN202364 (Origene), and multiple CRISPR products from GenScript (Piscataway, NJ). It is to be noted that the term can further be used to refer to any combination of features described herein regarding DPF2 molecules. For example, any combination of sequence composition, percentage identify, sequence length, domain structure, functional activity, etc. can be used to describe a DPF2 molecule encompassed by the present invention.

The term “DPF3” refers to Double PHD Fingers 3, a member of the D4 protein family. The encoded protein is a transcription regulator that binds acetylated histones and is a component of the BAF chromatin remodeling complex. DPF3 belongs to the neuron-specific chromatin remodeling complex (nBAF complex). During neural development a switch from a stem/progenitor to a post-mitotic chromatin remodeling mechanism occurs as neurons exit the cell cycle and become committed to their adult state. The transition from proliferating neural stem/progenitor cells to post-mitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth (By similarity). DPF3 is a muscle-specific component of the BAF complex, a multiprotein complex involved in transcriptional activation and repression of select genes by chromatin remodeling (alteration of DNA-nucleosome topology). DPF3 specifically binds acetylated lysines on histone 3 and 4 (H3K14ac, H3K9ac, H4K5ac, H4K8ac, H4K12ac, H4K16ac). In the complex, DPF3 acts as a tissue-specific anchor between histone acetylations and methylations and chromatin remodeling. DPF3 plays an essential role in heart and skeletal muscle development. Human DPF3 protein has 378 amino acids and a molecular mass of 43084 Da. The PHD-type zinc fingers of DPF3 mediate its binding to acetylated histones. DPF3 belongs to the requiem/DPF family.

The term “DPF3” is intended to include fragments, variants (e.g., allelic variants), and derivatives thereof. Representative human DPF3 cDNA and human DPF3 protein sequences are well-known in the art and are publicly available from the National Center for Biotechnology Information (NCBI). For example, four different human DPF3 isoforms are known. Human DPF3 isoform 1 (NP_036206.3) is encodable by the transcript variant 1 (NM_012074.4). Human DPF3 isoform 2 (NP_001267471.1) is encodable by the transcript variant 2 (NM_001280542.1). Human DPF3 isoform 3 (NP_001267472.1) is encodable by the transcript variant 3 (NM_001280543.1). Human DPF3 isoform 4 (NP_001267473.1) is encodable by the transcript variant 4 (NM_001280544.1). Nucleic acid and polypeptide sequences of DPF3 orthologs in organisms other than humans are well known and include, for example, chimpanzee DPF3 (XM_016926314.2 and XP_016781803.1, XM_016926316.2 and XP_016781805.1, and XM_016926315.2 and XP_016781804.1), dog DPF3 (XM_014116039.1 and XP_013971514.1), mouse DPF3 (NM_001267625.1 and NP_001254554.1, NM_001267626.1 and NP_001254555.1, and NM_058212.2 and NP_478119.1), chicken DPF3 (NM_204639.2 and NP_989970.1), tropical clawed frog DPF3 (NM_001278413.1 and NP_001265342.1), and zebrafish DPF3 (NM_001111169.1 and NP_001104639.1). Representative sequences of DPF3 orthologs are presented below in Table 1.

Anti-DPF3 antibodies suitable for detecting DPF3 protein are well-known in the art and include, for example, antibody TA335655 (Origene), antibodies NBP2-49494 and NBP2-14910 (Novus Biologicals, Littleton, CO), antibodies ab180914, ab127703, and ab85360 (AbCam, Cambridge, MA), antibody PA5-38011 (ThermoFisher Scientific), antibody Cat #7559 (ProSci, Poway, CA), etc. In addition, reagents are well-known for detecting DPF3. Moreover, mutilple siRNA, shRNA, CRISPR constructs for reducing DPF3 expression can be found in the commercial product lists of the above-referenced companies, such as siRNA products #sc-97031 and sc-92150 and CRISPR product #sc-143157 from Santa Cruz Biotechnology, RNAi products SR305368 and TL313386V, and CRISPR product KN218937 (Origene), and multiple CRISPR products from GenScript (Piscataway, NJ). It is to be noted that the term can further be used to refer to any combination of features described herein regarding DPF3 molecules. For example, any combination of sequence composition, percentage identify, sequence length, domain structure, functional activity, etc. can be used to describe a DPF3 molecule encompassed by the present invention.

The term “ACTL6A” refers to Actin Like 6A, a family member of actin-related proteins (ARPs), which share significant amino acid sequence identity to conventional actins. Both actins and ARPs have an actin fold, which is an ATP-binding cleft, as a common feature. The ARPs are involved in diverse cellular processes, including vesicular transport, spindle orientation, nuclear migration and chromatin remodeling. This gene encodes a 53 kDa subunit protein of the BAF (BRG1/brm-associated factor) complex in mammals, which is functionally related to SWI/SNF complex in S. cerevisiae and Drosophila; the latter is thought to facilitate transcriptional activation of specific genes by antagonizing chromatin-mediated transcriptional repression. Together with beta-actin, it is required for maximal ATPase activity of BRG1, and for the association of the BAF complex with chromatin/matrix. ACTL6A is a component of SWI/SNF chromatin remodeling complexes that carry out key enzymatic activities, changing chromatin structure by altering DNA-histone contacts within a nucleosome in an ATP-dependent manner. ACTL6A is required for maximal ATPase activity of SMARCA4/BRG1/BAF190A and for association of the SMARCA4/BRG1/BAF190A containing remodeling complex BAF with chromatin/nuclear matrix. ACTL6A belongs to the neural progenitors-specific chromatin remodeling complex (npBAF complex) and is required for the proliferation of neural progenitors. During neural development a switch from a stem/progenitor to a post-mitotic chromatin remodeling mechanism occurs as neurons exit the cell cycle and become committed to their adult state. The transition from proliferating neural stem/progenitor cells to post-mitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. ACTL6A is a component of the NuA4 histone acetyltransferase (HAT) complex which is involved in transcriptional activation of select genes principally by acetylation of nucleosomal histones H4 and H2A. This modification may both alter nucleosome—DNA interactions and promote interaction of the modified histones with other proteins which positively regulate transcription. This complex may be required for the activation of transcriptional programs associated with oncogene and proto-oncogene mediated growth induction, tumor suppressor mediated growth arrest and replicative senescence, apoptosis, and DNA repair. NuA4 may also play a direct role in DNA repair when recruited to sites of DNA damage. Putative core component of the chromatin remodeling IN080 complex which is involved in transcriptional regulation, DNA replication and probably DNA repair. Human ACTL6A protein has 429 amino acids and a molecular mass of 47461 Da.

The term “ACTL6A” is intended to include fragments, variants (e.g., allelic variants), and derivatives thereof. Representative human ACTL6A cDNA and human ACTL6A protein sequences are well-known in the art and are publicly available from the National Center for Biotechnology Information (NCBI). For example, two different human ACTL6A isoforms are known. Human ACTL6A isoform 1 (NP_004292.1) is encodable by the transcript variant 1 (NM_004301.4). Human ACTL6A isoform 2 (NP_817126.1 and NP_829888.1) is encodable by the transcript variant 2 (NM_177989.3) and transcript variant 3 (NM_178042.3). Nucleic acid and polypeptide sequences of ACTL6A orthologs in organisms other than humans are well known and include, for example, chimpanzee ACTL6A (NM_001271671.1 and NP_001258600.1), Rhesus monkey ACTL6A (NM_001104559.1 and NP_001098029.1), cattle ACTL6A (NM_001105035.1 and NP_001098505.1), mouse ACTL6A (NM_019673.2 and NP_062647.2), rat ACTL6A (NM_001039033.1 and NP_001034122.1), chicken ACTL6A (XM_422784.6 and XP_422784.3), tropical clawed frog ACTL6A (NM_204006.1 and NP_989337.1), and zebrafish ACTL6A (NM_173240.1 and NP_775347.1). Representative sequences of ACTL6A orthologs are presented below in Table 1.

Anti-ACTL6A antibodies suitable for detecting ACTL6A protein are well-known in the art and include, for example, antibody TA345058 (Origene), antibodies NB100-61628 and NBP2-55376 (Novus Biologicals, Littleton, CO), antibodies ab131272 and ab189315 (AbCam, Cambridge, MA), antibody 702414 (ThermoFisher Scientific), antibody Cat #45-314 (ProSci, Poway, CA), etc. In addition, reagents are well-known for detecting ACTL6A. Moreover, mutilple siRNA, shRNA, CRISPR constructs for reducing ACTL6A expression can be found in the commercial product lists of the above-referenced companies, such as siRNA products #sc-60239 and sc-60240 and CRISPR product #sc-403200-KO-2 from Santa Cruz Biotechnology, RNAi products SR300052 and TL306860V, and CRISPR product KN201689 (Origene), and multiple CRISPR products from GenScript (Piscataway, NJ). It is to be noted that the term can further be used to refer to any combination of features described herein regarding ACTL6A molecules. For example, any combination of sequence composition, percentage identify, sequence length, domain structure, functional activity, etc. can be used to describe an ACTL6A molecule encompassed by the present invention.

The term “β-Actin” refers to Actin Beta. This gene encodes one of six different actin proteins. Actins are highly conserved proteins that are involved in cell motility, structure, integrity, and intercellular signaling. The encoded protein is a major constituent of the contractile apparatus and one of the two nonmuscle cytoskeletal actins that are ubiquitously expressed. Mutations in this gene cause Baraitser-Winter syndrome 1, which is characterized by intellectual disability with a distinctive facial appearance in human patients. Numerous pseudogenes of this gene have been identified throughout the human genome. Actins are highly conserved proteins that are involved in various types of cell motility and are ubiquitously expressed in all eukaryotic cells. Actin is found in two main states: G-actin is the globular monomeric form, whereas F-actin forms helical polymers. Both G- and F-actin are intrinsically flexible structures. Human β-Actin protein has 375 amino acids and a molecular mass of 41737 Da. The binding partners of β-Actin include, e.g., CPNE1, CPNE4, DHX9, GCSAM, ERBB2, XPO6, and EMD.

The term “β-Actin” is intended to include fragments, variants (e.g., allelic variants), and derivatives thereof. Representative human β-Actin cDNA and human β-Actin protein sequences are well-known in the art and are publicly available from the National Center for Biotechnology Information (NCBI). For example, human β-Actin (NP_001092.1) is encodable by the transcript (NM_001101.4). Nucleic acid and polypeptide sequences of $3-Actin orthologs in organisms other than humans are well known and include, for example, chimpanzee β-Actin (NM_001009945.1 and NP_001009945.1), Rhesus monkey β-Actin (NM_001033084.1 and NP_001028256.1), dog β-Actin (NM_001195845.2 and NP_001182774.2), cattle β-Actin (NM_173979.3 and NP_776404.2), mouse β-Actin (NM_007393.5 and NP_031419.1), rat β-Actin (NM_031144.3 and NP_112406.1), chicken β-Actin (NM_205518.1 and NP_990849.1), and tropical clawed frog β-Actin (NM_213719.1 and NP_998884.1). Representative sequences of β-Actin orthologs are presented below in Table 1.

Anti-β-Actin antibodies suitable for detecting β-Actin protein are well-known in the art and include, for example, antibody TA353557 (Origene), antibodies NB600-501 and NB600-503 (Novus Biologicals, Littleton, CO), antibodies ab8226 and ab8227 (AbCam, Cambridge, MA), antibody AM4302 (ThermoFisher Scientific), antibody Cat #PM-7669-biotin (ProSci, Poway, CA), etc. In addition, reagents are well-known for detecting $-Actin. Moreover, mutilple siRNA, shRNA, CRISPR constructs for reducing β-Actin expression can be found in the commercial product lists of the above-referenced companies, such as siRNA products #sc-108069 and sc-108070 and CRISPR product #sc-400000-KO-2 from Santa Cruz Biotechnology, RNAi products SR300047 and TL314976V, and CRISPR product KN203643 (Origene), and multiple CRISPR products from GenScript (Piscataway, NJ). It is to be noted that the term can further be used to refer to any combination of features described herein regarding β-Actin molecules. For example, any combination of sequence composition, percentage identify, sequence length, domain structure, functional activity, etc. can be used to describe a β-Actin molecule encompassed by the present invention.

The term “BCL7A” refers to BCL Tumor Suppressor 7A. This gene is directly involved, with Myc and IgH, in a three-way gene translocation in a Burkitt lymphoma cell line. As a result of the gene translocation, the N-terminal region of the gene product is disrupted, which is thought to be related to the pathogenesis of a subset of high-grade B cell non-Hodgkin lymphoma. The N-terminal segment involved in the translocation includes the region that shares a strong sequence similarity with those of BCL7B and BCL7C. Diseases associated with BCL7A include Lymphoma and Burkitt Lymphoma. An important paralog of this gene is BCL7C. Human BCL7A protein has 210 amino acids and a molecular mass of 22810 Da.

The term “BCL7A” is intended to include fragments, variants (e.g., allelic variants), and derivatives thereof. Representative human BCL7A cDNA and human BCL7A protein sequences are well-known in the art and are publicly available from the National Center for Biotechnology Information (NCBI). For example, two different human BCL7A isoforms are known. Human BCL7A isoform a (NP_066273.1) is encodable by the transcript variant 1 (NM_020993.4). Human BCL7A isoform b (NP_001019979.1) is encodable by the transcript variant 2 (NM_001024808.2). Nucleic acid and polypeptide sequences of BCL7A orthologs in organisms other than humans are well known and include, for example, chimpanzee BCL7A (XM_009426452.3 and XP_009424727.2, and XM_016924434.2 and XP_016779923.1), Rhesus monkey BCL7A (XM_015153012.1 and XP_015008498.1, and XM_015153013.1 and XP_015008499.1), dog BCL7A (XM_543381.6 and XP_543381.2, and XM_854760.5 and XP_859853.1), cattle BCL7A (XM_024977701.1 and XP_024833469.1, and XM_024977700.1 and XP_024833468.1), mouse BCL7A (NM_029850.3 and NP_084126.1), rat BCL7A (XM_017598515.1 and XP_017454004.1), chicken BCL7A (XM_004945565.3 and XP_004945622.1, and XM_415148.6 and XP_415148.2), tropical clawed frog BCL7A (NM_001006871.1 and NP_001006872.1), and zebrafish BCL7A (NM_212560.1 and NP_997725.1). Representative sequences of BCL7A orthologs are presented below in Table 1.

Anti-BCL7A antibodies suitable for detecting BCL7A protein are well-known in the art and include, for example, antibody TA344744 (Origene), antibodies NBP1-30941 and NBP1-91696 (Novus Biologicals, Littleton, CO), antibodies ab137362 and ab1075 (AbCam, Cambridge, MA), antibody PA5-27123 (ThermoFisher Scientific), antibody Cat #45-325 (ProSci, Poway, CA), etc. In addition, reagents are well-known for detecting BCL7A. Multiple clinical tests of BCL7A are available in NIH Genetic Testing Registry (GTR®) (e.g., GTR Test ID: GTR000541481.2, offered by Fulgent Clinical Diagnostics Lab (Temple City, CA)). Moreover, mutilple siRNA, shRNA, CRISPR constructs for reducing BCL7A expression can be found in the commercial product lists of the above-referenced companies, such as siRNA products #sc-96136 and sc-141671 and CRISPR product #sc-410702 from Santa Cruz Biotechnology, RNAi products SR300417 and TL314490V, and CRISPR product KN210489 (Origene), and multiple CRISPR products from GenScript (Piscataway, NJ). It is to be noted that the term can further be used to refer to any combination of features described herein regarding BCL7A molecules. For example, any combination of sequence composition, percentage identify, sequence length, domain structure, functional activity, etc. can be used to describe a BCL7A molecule encompassed by the present invention.

The term “BCL7B” refers to BCL Tumor Suppressor 7B, a member of the BCL7 family including BCL7A, BCL7B and BCL7C proteins. This member is BCL7B, which contains a region that is highly similar to the N-terminal segment of BCL7A or BCL7C proteins. The BCL7A protein is encoded by the gene known to be directly involved in a three-way gene translocation in a Burkitt lymphoma cell line. This gene is located at a chromosomal region commonly deleted in Williams syndrome. This gene is highly conserved from C. elegans to human. BCL7B is a positive regulator of apoptosis. BCL7B plays a role in the Wnt signaling pathway, negatively regulating the expression of Wnt signaling components CTNNB1 and HMGA1 (Uehara et al. (2015) PLoS Genet 11(1):e1004921). BCL7B is involved in cell cycle progression, maintenance of the nuclear structure and stem cell differentiation (Uehara et al. (2015) PLoS Genet 11(1):e1004921). It plays a role in lung tumor development or progression. Human BCL7B protein has 202 amino acids and a molecular mass of 22195 Da.

The term “BCL7B” is intended to include fragments, variants (e.g., allelic variants), and derivatives thereof. Representative human BCL7B cDNA and human BCL7B protein sequences are well-known in the art and are publicly available from the National Center for Biotechnology Information (NCBI). For example, three different human BCL7B isoforms are known. Human BCL7B isoform 1 (NP_001698.2) is encodable by the transcript variant 1 (NM_001707.3). Human BCL7B isoform 2 (NP_001184173.1) is encodable by the transcript variant 2 (NM_001197244.1). Human BCL7B isoform 3 (NP_001287990.1) is encodable by the transcript variant 3 (NM_001301061.1). Nucleic acid and polypeptide sequences of BCL7B orthologs in organisms other than humans are well known and include, for example, chimpanzee BCL7B (XM_003318671.3 and XP_003318719.1, and XM_003318672.3 and XP_003318720.1), Rhesus monkey BCL7B (NM_001194509.1 and NP_001181438.1), dog BCL7B (XM_546926.6 and XP_546926.1, and XM_005620975.2 and XP_005621032.1), cattle BCL7B (NM_001034775.2 and NP_001029947.1), mouse BCL7B (NM_009745.2 and NP_033875.2), chicken BCL7B (XM_003643231.4 and XP_003643279.1, XM_004949975.3 and XP_004950032.1, and XM_025142155.1 and XP_024997923.1), tropical clawed frog BCL7B (NM_001103072.1 and NP_001096542.1), and zebrafish BCL7B (NM_001006018.1 and NP_001006018.1, and NM_213165.1 and NP_998330.1). Representative sequences of BCL7B orthologs are presented below in Table 1.

Anti-BCL7B antibodies suitable for detecting BCL7B protein are well-known in the art and include, for example, antibody TA809485 (Origene), antibodies H00009275-M01 and NBP2-34097 (Novus Biologicals, Littleton, CO), antibodies ab130538 and ab172358 (AbCam, Cambridge, MA), antibody MA527163 (ThermoFisher Scientific), antibody Cat #58-996 (ProSci, Poway, CA), etc. In addition, reagents are well-known for detecting BCL7B. Moreover, mutilple siRNA, shRNA, CRISPR constructs for reducing BCL7B expression can be found in the commercial product lists of the above-referenced companies, such as siRNA products #sc-89728 and sc-141672 and CRISPR product #sc-411262 from Santa Cruz Biotechnology, RNAi products SR306141 and TL306418V, and CRISPR product KN201696 (Origene), and multiple CRISPR products from GenScript (Piscataway, NJ). It is to be noted that the term can further be used to refer to any combination of features described herein regarding BCL7B molecules. For example, any combination of sequence composition, percentage identify, sequence length, domain structure, functional activity, etc. can be used to describe a BCL7B molecule encompassed by the present invention.

The term “BCL7C” refers to BCL Tumor Suppressor 7C, a member of the BCL7 family including BCL7A, BCL7B and BCL7C proteins. This gene is identified by the similarity of its product to the N-terminal region of BCL7A protein. BCL7C may play an anti-apoptotic role. Diseases associated with BCL7C include Lymphoma. Human BCL7C protein has 217 amino acids and a molecular mass of 23468 Da.

The term “BCL7C” is intended to include fragments, variants (e.g., allelic variants), and derivatives thereof. Representative human BCL7C cDNA and human BCL7C protein sequences are well-known in the art and are publicly available from the National Center for Biotechnology Information (NCBI). For example, two different human BCL7C isoforms are known. Human BCL7C isoform 1 (NP_001273455.1) is encodable by the transcript variant 1 (NM_001286526.1). Human BCL7C isoform 2 (NP_004756.2) is encodable by the transcript variant 2 (NM_004765.3). Nucleic acid and polypeptide sequences of BCL7C orthologs in organisms other than humans are well known and include, for example, chimpanzee BCL7C (XM_016929717.2 and XP_016785206.1, XM_016929716.2 and XP_016785205.1, and XM_016929718.2 and XP_016785207.1), Rhesus monkey BCL7C (NM_001265776.2 and NP_001252705.1), cattle BCL7C (NM_001099722.1 and NP_001093192.1), mouse BCL7C (NM_001347652.1 and NP_001334581.1, and NM_009746.2 and NP_033876.1), and rat BCL7C (NM_001106298.1 and NP_001099768.1). Representative sequences of BCL7C orthologs are presented below in Table 1.

Anti-BCL7C antibodies suitable for detecting BCL7C protein are well-known in the art and include, for example, antibody TA347083 (Origene), antibodies NBP2-15559 and NBP1-86441 (Novus Biologicals, Littleton, CO), antibodies ab126944 and ab231278 (AbCam, Cambridge, MA), antibody PA5-30308 (ThermoFisher Scientific), etc. In addition, reagents are well-known for detecting BCL7C. Multiple clinical tests of BCL7C are available in NIH Genetic Testing Registry (GTR®) (e.g., GTR Test ID: GTR000540637.2, offered by Fulgent Clinical Diagnostics Lab (Temple City, CA)). Moreover, mutilple siRNA, shRNA, CRISPR constructs for reducing BCL7C expression can be found in the commercial product lists of the above-referenced companies, such as siRNA products #sc-93022 and sc-141673 and CRISPR product #sc-411261 from Santa Cruz Biotechnology, RNAi products SR306140 and TL315552V, and CRISPR product KN205720 (Origene), and multiple CRISPR products from GenScript (Piscataway, NJ). It is to be noted that the term can further be used to refer to any combination of features described herein regarding BCL7C molecules. For example, any combination of sequence composition, percentage identify, sequence length, domain structure, functional activity, etc. can be used to describe a BCL7C molecule encompassed by the present invention.

The term “SMARCA4” refers to SWI/SNF related, matrix associated, actin dependent regulator of chromatin, subfamily a, member 4, a member of the SWI/SNF family of proteins and is highly similar to the brahma protein of Drosophila. Members of this family have helicase and ATPase activities and are thought to regulate transcription of certain genes by altering the chromatin structure around those genes. The encoded protein is part of the large ATP-dependent chromatin remodeling complex SNF/SWI, which is required for transcriptional activation of genes normally repressed by chromatin. In addition, this protein can bind BRCA1, as well as regulate the expression of the tumorigenic protein CD44. Mutations in this gene cause rhabdoid tumor predisposition syndrome type 2. SMARCA4 is a component of SWI/SNF chromatin remodeling complexes that carry out key enzymatic activities, changing chromatin structure by altering DNA-histone contacts within a nucleosome in an ATP-dependent manner. SMARCA4 is a component of the CREST-BRG1 complex, a multiprotein complex that regulates promoter activation by orchestrating a calcium-dependent release of a repressor complex and a recruitment of an activator complex. In resting neurons, transcription of the c-FOS promoter is inhibited by BRG1-dependent recruitment of a phospho-RB1-HDAC repressor complex. Upon calcium influx, RB1 is dephosphorylated by calcineurin, which leads to release of the repressor complex. At the same time, there is increased recruitment of CREBBP to the promoter by a CREST-dependent mechanism, which leads to transcriptional activation. The CREST-BRG1 complex also binds to the NR2B promoter, and activity-dependent induction of NR2B expression involves a release of HDAC1 and recruitment of CREBBP. SMARCA4 belongs to the neural progenitors-specific chromatin remodeling complex (npBAF complex) and the neuron-specific chromatin remodeling complex (nBAF complex). During neural development a switch from a stem/progenitor to a postmitotic chromatin remodeling mechanism occurs as neurons exit the cell cycle and become committed to their adult state. The transition from proliferating neural stem/progenitor cells to postmitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. SMARCA4/BAF190A promote neural stem cell self-renewal/proliferation by enhancing Notch-dependent proliferative signals, while concurrently making the neural stem cell insensitive to SHH-dependent differentiating cues. SMARCA4 acts as a corepressor of ZEB1 to regulate E-cadherin transcription and is required for induction of epithelial-mesenchymal transition (EMT) by ZEB1. Human SMARCA4 protein has 1647 amino acids and a molecular mass of 184646 Da. The known binding partners of SMARCA4 include, e.g., PHF10/BAF45A, MYOG, IKFZ1, ZEB1, NR3C1, PGR, SMARD1, TOPBP1 and ZMIM2/ZIMP7.

The term “SMARCA4” is intended to include fragments, variants (e.g., allelic variants), and derivatives thereof. Representative human SMARCA4 cDNA and human SMARCA4 protein sequences are well-known in the art and are publicly available from the National Center for Biotechnology Information (NCBI). For example, six different human SMARCA4 isoforms are known. Human SMARCA4 isoform A (NP_001122321.1) is encodable by the transcript variant 1 (NM_001128849.1). Human SMARCA4 isoform B (NP_001122316.1 and NP_003063.2) is encodable by the transcript variant 2 (NM_001128844.1) and the transcript variant 3 (NM_003072.3). Human SMARCA4 isoform C (NP_001122317.1) is encodable by the transcript variant 4 (NM_001128845.1). Human SMARCA4 isoform D (NP_001122318.1) is encodable by the transcript variant 5 (NM_001128846.1). Human SMARCA4 isoform E (NP_001122319.1) is encodable by the transcript variant 6 (NM_001128847.1). Human SMARCA4 isoform F (NP_001122320.1) is encodable by the transcript variant 7 (NM_001128848.1). Nucleic acid and polypeptide sequences of SMARCA4 orthologs in organisms other than humans are well known and include, for example, Rhesus monkey SMARCA4 (XM_015122901.1 and XP_014978387.1, XM_015122902.1 and XP_014978388.1, XM_015122903.1 and XP_014978389.1, XM_015122906.1 and XP_014978392.1, XM_015122905.1 and XP_014978391.1, XM_015122904.1 and XP_014978390.1, XM_015122907.1 and XP_014978393.1, XM_015122909.1 and XP_014978395.1, and XM_015122910.1 and XP_014978396.1), cattle SMARCA4 (NM_001105614.1 and NP_001099084.1), mouse SMARCA4 (NM_001174078.1 and NP_001167549.1, NM_011417.3 and NP 035547.2, NM_001174079.1 and NP_001167550.1, NM_001357764.1 and NP_001344693.1), rat SMARCA4 (NM_134368.1 and NP_599195.1), chicken SMARCA4 (NM_205059.1 and NP_990390.1), and zebrafish SMARCA4 (NM_181603.1 and NP_853634.1). Representative sequences of SMARCA4 orthologs are presented below in Table 1.

Anti-SMARCA4 antibodies suitable for detecting SMARCA4 protein are well-known in the art and include, for example, antibody AM26021PU-N(Origene), antibodies NB100-2594 and AF5738 (Novus Biologicals, Littleton, CO), antibodies ab110641 and ab4081 (AbCam, Cambridge, MA), antibody 720129 (ThermoFisher Scientific), antibody 7749 (ProSci), etc. In addition, reagents are well-known for detecting SMARCA4. Multiple clinical tests of SMARCA4 are available in NIH Genetic Testing Registry (GTR®) (e.g., GTR Test ID: GTR000517106.2, offered by Fulgent Clinical Diagnostics Lab (Temple City, CA)). Moreover, mutilple siRNA, shRNA, CRISPR constructs for reducing SMARCA4 expression can be found in the commercial product lists of the above-referenced companies, such as siRNA products #sc-29827 and sc-44287 and CRISPR product #sc-400168 from Santa Cruz Biotechnology, RNAi products SR321835 and TL309249V, and CRISPR product KN219258 (Origene), and multiple CRISPR products from GenScript (Piscataway, NJ). It is to be noted that the term can further be used to refer to any combination of features described herein regarding SMARCA4 molecules. For example, any combination of sequence composition, percentage identify, sequence length, domain structure, functional activity, etc. can be used to describe a SMARCA4 molecule encompassed by the present invention.

The term “SMARCE1” refers to SWI/SNF related, matrix associated, actin dependent regulator of chromatin subfamily E member 1. The protein encoded by this gene is part of the large ATP-dependent chromatin remodeling complex SWI/SNF, which is required for transcriptional activation of genes normally repressed by chromatin. The encoded protein, either alone or when in the SWI/SNF complex, can bind to 4-way junction DNA, which is thought to mimic the topology of DNA as it enters or exits the nucleosome. The protein contains a DNA-binding HMG domain, but disruption of this domain does not abolish the DNA-binding or nucleosome-displacement activities of the SWI/SNF complex. Unlike most of the SWI/SNF complex proteins, this protein has no yeast counterpart. SMARCE1 is a component of SWI/SNF chromatin remodeling complexes that carry out key enzymatic activities, changing chromatin structure by altering DNA-histone contacts within a nucleosome in an ATP-dependent manner. SMARCE1 belongs to the neural progenitors-specific chromatin remodeling complex (npBAF complex) and the neuron-specific chromatin remodeling complex (nBAF complex). SMARCE1 is required for the coactivation of estrogen responsive promoters by SWI/SNF complexes and the SRC/p160 family of histone acetyltransferases (HATs). SMARCE1 also specifically interacts with the CoREST corepressor resulting in repression of neuronal specific gene promoters in non-neuronal cells. Human SMARCE1 protein has 411 amino acids and a molecular mass of 46649 Da. SMARCE1 interacts with BRDT, and also binds to the SRC/p160 family of histone acetyltransferases (HATs) composed of NCOA1, NCOA2, and NCOA3. SMARCE1 interacts with RCOR1/CoREST, NR3C1 and ZMIM2/ZIMP7.

The term “SMARCE1” is intended to include fragments, variants (e.g., allelic variants), and derivatives thereof. Representative human SMARCE1 cDNA and human SMARCE1 protein sequences are well-known in the art and are publicly available from the National Center for Biotechnology Information (NCBI). For example, human SMARCE1 protein (NP_003070.3) is encodable by transcript (NM_003079.4). Nucleic acid and polypeptide sequences of SMARCE1 orthologs in organisms other than humans are well known and include, for example, chimpanzee SMARCE1 (XM_009432223.3 and XP_009430498.1, XM_511478.7 and XP_511478.2, XM_009432222.3 and XP_009430497.1, and XM_001169953.6 and XP_001169953.1), Rhesus monkey SMARCE1 (NM_001261306.1 and NP_001248235.1), cattle SMARCE1 (NM_001099116.2 and NP_001092586.1), mouse SMARCE1 (NM_020618.4 and NP_065643.1), rat SMARCE1 (NM_001024993.1 and NP_001020164.1), chicken SMARCE1 (NM_001006335.2 and NP_001006335.2), tropical clawed frog SMARCE1 (NM_001005436.1 and NP_001005436.1), and zebrafish SMARCE1 (NM_201298.1 and NP_958455.2). Representative sequences of SMARCE1 orthologs are presented below in Table 1.

Anti-SMARCE1 antibodies suitable for detecting SMARCE1 protein are well-known in the art and include, for example, antibody TA335790 (Origene), antibodies NBP1-90012 and NB100-2591 (Novus Biologicals, Littleton, CO), antibodies ab131328, ab228750, and ab137081 (AbCam, Cambridge, MA), antibody Cat #PA5-18185 (ThermoFisher Scientific), antibody Cat #57-670 (ProSci, Poway, CA), etc. In addition, reagents are well-known for detecting SMARCE1. A clinical test of SMARCE1 for hereditary disese is available with the test ID no. GTR000558444.1 in NIH Genetic Testing Registry (GTR®), offered by Tempus Labs, Inc., (Chicago, IL). Moreover, mutilple siRNA, shRNA, CRISPR constructs for reducing SMARCE1 expression can be found in the commercial product lists of the above-referenced companies, such as siRNA products #sc-45940 and sc-45941 and CRISPR product #sc-404713 from Santa Cruz Biotechnology, RNAi products SR304479 and TL309242, and CRISPR product KN217885 (Origene), and multiple CRISPR products from GenScript (Piscataway, NJ). It is to be noted that the term can further be used to refer to any combination of features described herein regarding SMARCE1 molecules. For example, any combination of sequence composition, percentage identify, sequence length, domain structure, functional activity, etc. can be used to describe a SMARCE1 molecule encompassed by the present invention.

The term “DPF1” refers to Double PHD Fingers 1. DPF1 has an important role in developing neurons by participating in regulation of cell survival, possibly as a neurospecific transcription factor. DPF1 belongs to the neuron-specific chromatin remodeling complex (nBAF complex). During neural development a switch from a stem/progenitor to a post-mitotic chromatin remodeling mechanism occurs as neurons exit the cell cycle and become committed to their adult state. The transition from proliferating neural stem/progenitor cells to post-mitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. Human DPF1 protein has 380 amino acids and a molecular mass of 425029 Da. DPF1 is a component of neuron-specific chromatin remodeling complex (nBAF complex) composed of at least, ARID1A/BAF250A or ARID1B/BAF250B, SMARCD1/BAF60A, SMARCD3/BAF60C, SMARCA2/BRM/BAF190B, SMARCA4/BRG1/BAF190A, SMARCB1/BAF47, SMARCC1/BAF155, SMARCE1/BAF57, SMARCC2/BAF170, DPF1/BAF45B, DPF3/BAF45C, ACTL6B/BAF53B and actin.

The term “DPF1” is intended to include fragments, variants (e.g., allelic variants), and derivatives thereof. Representative human DPF1 cDNA and human DPF1 protein sequences are well-known in the art and are publicly available from the National Center for Biotechnology Information (NCBI). For example, five different human DPF1 isoforms are known. Human DPF1 isoform a (NP_001128627.1) is encodable by the transcript variant 1 (NM_001135155.2). Human DPF1 isoform b (NP_004638.2) is encodable by the transcript variant 2 (NM_004647.3). Human DPF1 isoform c (NP_001128628.1) is encodable by the transcript variant 3 (NM_001135156.2). Human DPF1 isoform d (NP_001276907.1) is encodable by the transcript variant 4 (NM_001289978.1). Human DPF1 isoform e (NP_001350508.1) is encodable by the transcript variant 5 (NM_001363579.1). Nucleic acid and polypeptide sequences of DPF1 orthologs in organisms other than humans are well known and include, for example, Rhesus monkey DPF1 (XM_015123830.1 and XP_014979316.1, XM_015123829.1 and XP_014979315.1, XM_015123835.1 and XP_014979321.1, XM_015123831.1 and XP_014979317.1, XM_015123833.1 and XP_014979319.1, and XM_015123832.1 and XP_014979318.1), cattle DPF1 (NM_001076855.1 and NP_001070323.1), mouse DPF1 (NM_013874.2 and NP_038902.1), rat DPF1 (NM_001105729.3 and NP_001099199.2), and tropical clawed frog DPF1 (NM_001097276.1 and NP_001090745.1). Representative sequences of DPF1 orthologs are presented below in Table 1.

Anti-DPF1 antibodies suitable for detecting DPF1 protein are well-known in the art and include, for example, antibody TA311193 (Origene), antibodies NBP2-13932 and NBP2-19518 (Novus Biologicals, Littleton, CO), antibodies ab199299, ab173160, and ab3940 (AbCam, Cambridge, MA), antibody Cat #PA5-61895 (ThermoFisher Scientific), antibody Cat #28-079 (ProSci, Poway, CA), etc. In addition, reagents are well-known for detecting DPF1. Moreover, mutilple siRNA, shRNA, CRISPR constructs for reducing DPF1 expression can be found in the commercial product lists of the above-referenced companies, such as siRNA products #sc-97084 and sc-143155 and CRISPR product #sc-409539 from Santa Cruz Biotechnology, RNAi products SR305389 and TL313388V, and CRISPR product KN213721 (Origene), and multiple CRISPR products from GenScript (Piscataway, NJ). It is to be noted that the term can further be used to refer to any combination of features described herein regarding DPF1 molecules. For example, any combination of sequence composition, percentage identify, sequence length, domain structure, functional activity, etc. can be used to describe a DPF1 molecule encompassed by the present invention.

The term “DPF2” refers to Double PHD Fingers 2. DPF2 protein is a member of the d4 domain family, characterized by a zinc finger-like structural motif. It functions as a transcription factor which is necessary for the apoptotic response following deprivation of survival factors. It likely serves a regulatory role in rapid hematopoietic cell growth and turnover. This gene is considered a candidate gene for multiple endocrine neoplasia type I, an inherited cancer syndrome involving multiple parathyroid, enteropancreatic, and pituitary tumors. DPF2 is a transcription factor required for the apoptosis response following survival factor withdrawal from myeloid cells. DPF2 also has a role in the development and maturation of lymphoid cells. Human DPF2 protein has 391 amino acids and a molecular mass of 44155 Da.

The term “DPF2” is intended to include fragments, variants (e.g., allelic variants), and derivatives thereof. Representative human DPF2 cDNA and human DPF2 protein sequences are well-known in the art and are publicly available from the National Center for Biotechnology Information (NCBI). For example, two different human DPF2 isoforms are known. Human DPF2 isoform 1 (NP_006259.1) is encodable by the transcript variant 1 (NM_006268.4). Human DPF2 isoform 2 (NP_001317237.1) is encodable by the transcript variant 2 (NM_001330308.1). Nucleic acid and polypeptide sequences of DPF2 orthologs in organisms other than humans are well known and include, for example, chimpanzee DPF2 (NM_001246651.1 and NP_001233580.1), Rhesus monkey DPF2 (XM_002808062.2 and XP_002808108.2, and XM_015113800.1 and XP_014969286.1), dog DPF2 (XM_861495.5 and XP_866588.1, and XM_005631484.3 and XP_005631541.1), cattle DPF2 (NM_001100356.1 and NP_001093826.1), mouse DPF2 (NM_001291078.1 and NP_001278007.1, and NM_011262.5 and NP_035392.1), rat DPF2 (NM_001108516.1 and NP_001101986.1), chicken DPF2 (NM_204331.1 and NP_989662.1), tropical clawed frog DPF2 (NM_001197172.2 and NP_001184101.1), and zebrafish DPF2 (NM_001007152.1 and NP_001007153.1). Representative sequences of DPF2 orthologs are presented below in Table 1.

Anti-DPF2 antibodies suitable for detecting DPF2 protein are well-known in the art and include, for example, antibody TA312307 (Origene), antibodies NBP1-76512 and NBP1-87138 (Novus Biologicals, Littleton, CO), antibodies ab134942, ab232327, and ab227095 (AbCam, Cambridge, MA), etc. In addition, reagents are well-known for detecting DPF2. A clinical test of DPF2 for hereditary disese is available with the test ID no. GTR000536833.2 in NIH Genetic Testing Registry (GTR®), offered by Fulgent Genetics Clinical Diagnostics Lab (Temple City, CA). Moreover, mutilple siRNA, shRNA, CRISPR constructs for reducing DPF2 expression can be found in the commercial product lists of the above-referenced companies, such as siRNA products #sc-97031 and sc-143156 and CRISPR product #sc-404801-KO-2 from Santa Cruz Biotechnology, RNAi products SR304035 and TL313387V, and CRISPR product KN202364 (Origene), and multiple CRISPR products from GenScript (Piscataway, NJ). It is to be noted that the term can further be used to refer to any combination of features described herein regarding DPF2 molecules. For example, any combination of sequence composition, percentage identify, sequence length, domain structure, functional activity, etc. can be used to describe a DPF2 molecule encompassed by the present invention.

The term “DPF3” refers to Double PHD Fingers 3, a member of the D4 protein family. The encoded protein is a transcription regulator that binds acetylated histones and is a component of the BAF chromatin remodeling complex. DPF3 belongs to the neuron-specific chromatin remodeling complex (nBAF complex). During neural development a switch from a stem/progenitor to a post-mitotic chromatin remodeling mechanism occurs as neurons exit the cell cycle and become committed to their adult state. The transition from proliferating neural stem/progenitor cells to post-mitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth (By similarity). DPF3 is a muscle-specific component of the BAF complex, a multiprotein complex involved in transcriptional activation and repression of select genes by chromatin remodeling (alteration of DNA-nucleosome topology). DPF3 specifically binds acetylated lysines on histone 3 and 4 (H3K14ac, H3K9ac, H4K5ac, H4K8ac, H4K12ac, H4K16ac). In the complex, DPF3 acts as a tissue-specific anchor between histone acetylations and methylations and chromatin remodeling. DPF3 plays an essential role in heart and skeletal muscle development. Human DPF3 protein has 378 amino acids and a molecular mass of 43084 Da. The PHD-type zinc fingers of DPF3 mediate its binding to acetylated histones. DPF3 belongs to the requiem/DPF family.

The term “DPF3” is intended to include fragments, variants (e.g., allelic variants), and derivatives thereof. Representative human DPF3 cDNA and human DPF3 protein sequences are well-known in the art and are publicly available from the National Center for Biotechnology Information (NCBI). For example, four different human DPF3 isoforms are known. Human DPF3 isoform 1 (NP_036206.3) is encodable by the transcript variant 1 (NM_012074.4). Human DPF3 isoform 2 (NP_001267471.1) is encodable by the transcript variant 2 (NM_001280542.1). Human DPF3 isoform 3 (NP_001267472.1) is encodable by the transcript variant 3 (NM_001280543.1). Human DPF3 isoform 4 (NP_001267473.1) is encodable by the transcript variant 4 (NM_001280544.1). Nucleic acid and polypeptide sequences of DPF3 orthologs in organisms other than humans are well known and include, for example, chimpanzee DPF3 (XM_016926314.2 and XP_016781803.1, XM_016926316.2 and XP_016781805.1, and XM_016926315.2 and XP_016781804.1), dog DPF3 (XM_014116039.1 and XP_013971514.1), mouse DPF3 (NM_001267625.1 and NP_001254554.1, NM_001267626.1 and NP_001254555.1, and NM_058212.2 and NP_478119.1), chicken DPF3 (NM_204639.2 and NP_989970.1), tropical clawed frog DPF3 (NM_001278413.1 and NP_001265342.1), and zebrafish DPF3 (NM_001111169.1 and NP_001104639.1). Representative sequences of DPF3 orthologs are presented below in Table 1.

Anti-DPF3 antibodies suitable for detecting DPF3 protein are well-known in the art and include, for example, antibody TA335655 (Origene), antibodies NBP2-49494 and NBP2-14910 (Novus Biologicals, Littleton, CO), antibodies ab180914, ab127703, and ab85360 (AbCam, Cambridge, MA), antibody PA5-38011 (ThermoFisher Scientific), antibody Cat #7559 (ProSci, Poway, CA), etc. In addition, reagents are well-known for detecting DPF3. Moreover, mutilple siRNA, shRNA, CRISPR constructs for reducing DPF3 expression can be found in the commercial product lists of the above-referenced companies, such as siRNA products #sc-97031 and sc-92150 and CRISPR product #sc-143157 from Santa Cruz Biotechnology, RNAi products SR305368 and TL313386V, and CRISPR product KN218937 (Origene), and multiple CRISPR products from GenScript (Piscataway, NJ). It is to be noted that the term can further be used to refer to any combination of features described herein regarding DPF3 molecules. For example, any combination of sequence composition, percentage identify, sequence length, domain structure, functional activity, etc. can be used to describe a DPF3 molecule encompassed by the present invention.

The term “ACTL6A” refers to Actin Like 6A, a family member of actin-related proteins (ARPs), which share significant amino acid sequence identity to conventional actins. Both actins and ARPs have an actin fold, which is an ATP-binding cleft, as a common feature. The ARPs are involved in diverse cellular processes, including vesicular transport, spindle orientation, nuclear migration and chromatin remodeling. This gene encodes a 53 kDa subunit protein of the BAF (BRG1/brm-associated factor) complex in mammals, which is functionally related to SWI/SNF complex in S. cerevisiae and Drosophila; the latter is thought to facilitate transcriptional activation of specific genes by antagonizing chromatin-mediated transcriptional repression. Together with beta-actin, it is required for maximal ATPase activity of BRG1, and for the association of the BAF complex with chromatin/matrix. ACTL6A is a component of SWI/SNF chromatin remodeling complexes that carry out key enzymatic activities, changing chromatin structure by altering DNA-histone contacts within a nucleosome in an ATP-dependent manner. ACTL6A is required for maximal ATPase activity of SMARCA4/BRG1/BAF190A and for association of the SMARCA4/BRG1/BAF190A containing remodeling complex BAF with chromatin/nuclear matrix. ACTL6A belongs to the neural progenitors-specific chromatin remodeling complex (npBAF complex) and is required for the proliferation of neural progenitors. During neural development a switch from a stem/progenitor to a post-mitotic chromatin remodeling mechanism occurs as neurons exit the cell cycle and become committed to their adult state. The transition from proliferating neural stem/progenitor cells to post-mitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. ACTL6A is a component of the NuA4 histone acetyltransferase (HAT) complex which is involved in transcriptional activation of select genes principally by acetylation of nucleosomal histones H4 and H2A. This modification may both alter nucleosome—DNA interactions and promote interaction of the modified histones with other proteins which positively regulate transcription. This complex may be required for the activation of transcriptional programs associated with oncogene and proto-oncogene mediated growth induction, tumor suppressor mediated growth arrest and replicative senescence, apoptosis, and DNA repair. NuA4 may also play a direct role in DNA repair when recruited to sites of DNA damage. Putative core component of the chromatin remodeling IN080 complex which is involved in transcriptional regulation, DNA replication and probably DNA repair. Human ACTL6A protein has 429 amino acids and a molecular mass of 47461 Da.

The term “ACTL6A” is intended to include fragments, variants (e.g., allelic variants), and derivatives thereof. Representative human ACTL6A cDNA and human ACTL6A protein sequences are well-known in the art and are publicly available from the National Center for Biotechnology Information (NCBI). For example, two different human ACTL6A isoforms are known. Human ACTL6A isoform 1 (NP_004292.1) is encodable by the transcript variant 1 (NM_004301.4). Human ACTL6A isoform 2 (NP_817126.1 and NP_829888.1) is encodable by the transcript variant 2 (NM_177989.3) and transcript variant 3 (NM_178042.3). Nucleic acid and polypeptide sequences of ACTL6A orthologs in organisms other than humans are well known and include, for example, chimpanzee ACTL6A (NM_001271671.1 and NP_001258600.1), Rhesus monkey ACTL6A (NM_001104559.1 and NP_001098029.1), cattle ACTL6A (NM_001105035.1 and NP_001098505.1), mouse ACTL6A (NM_019673.2 and NP_062647.2), rat ACTL6A (NM_001039033.1 and NP_001034122.1), chicken ACTL6A (XM_422784.6 and XP_422784.3), tropical clawed frog ACTL6A (NM_204006.1 and NP_989337.1), and zebrafish ACTL6A (NM_173240.1 and NP_775347.1). Representative sequences of ACTL6A orthologs are presented below in Table 1.

Anti-ACTL6A antibodies suitable for detecting ACTL6A protein are well-known in the art and include, for example, antibody TA345058 (Origene), antibodies NB100-61628 and NBP2-55376 (Novus Biologicals, Littleton, CO), antibodies ab131272 and ab189315 (AbCam, Cambridge, MA), antibody 702414 (ThermoFisher Scientific), antibody Cat #45-314 (ProSci, Poway, CA), etc. In addition, reagents are well-known for detecting ACTL6A. Moreover, mutilple siRNA, shRNA, CRISPR constructs for reducing ACTL6A expression can be found in the commercial product lists of the above-referenced companies, such as siRNA products #sc-60239 and sc-60240 and CRISPR product #sc-403200-KO-2 from Santa Cruz Biotechnology, RNAi products SR300052 and TL306860V, and CRISPR product KN201689 (Origene), and multiple CRISPR products from GenScript (Piscataway, NJ). It is to be noted that the term can further be used to refer to any combination of features described herein regarding ACTL6A molecules. For example, any combination of sequence composition, percentage identify, sequence length, domain structure, functional activity, etc. can be used to describe an ACTL6A molecule encompassed by the present invention.

The term “β-Actin” refers to Actin Beta. This gene encodes one of six different actin proteins. Actins are highly conserved proteins that are involved in cell motility, structure, integrity, and intercellular signaling. The encoded protein is a major constituent of the contractile apparatus and one of the two nonmuscle cytoskeletal actins that are ubiquitously expressed. Mutations in this gene cause Baraitser-Winter syndrome 1, which is characterized by intellectual disability with a distinctive facial appearance in human patients. Numerous pseudogenes of this gene have been identified throughout the human genome. Actins are highly conserved proteins that are involved in various types of cell motility and are ubiquitously expressed in all eukaryotic cells. Actin is found in two main states: G-actin is the globular monomeric form, whereas F-actin forms helical polymers. Both G- and F-actin are intrinsically flexible structures. Human β-Actin protein has 375 amino acids and a molecular mass of 41737 Da. The binding partners of β-Actin include, e.g., CPNE1, CPNE4, DHX9, GCSAM, ERBB2, XPO6, and EMD.

The term “β-Actin” is intended to include fragments, variants (e.g., allelic variants), and derivatives thereof. Representative human β-Actin cDNA and human β-Actin protein sequences are well-known in the art and are publicly available from the National Center for Biotechnology Information (NCBI). For example, human β-Actin (NP_001092.1) is encodable by the transcript (NM_001101.4). Nucleic acid and polypeptide sequences of $3-Actin orthologs in organisms other than humans are well known and include, for example, chimpanzee β-Actin (NM_001009945.1 and NP_001009945.1), Rhesus monkey β-Actin (NM_001033084.1 and NP_001028256.1), dog β-Actin (NM_001195845.2 and NP_001182774.2), cattle β-Actin (NM_173979.3 and NP_776404.2), mouse β-Actin (NM_007393.5 and NP_031419.1), rat β-Actin (NM_031144.3 and NP_112406.1), chicken β-Actin (NM_205518.1 and NP_990849.1), and tropical clawed frog β-Actin (NM_213719.1 and NP_998884.1). Representative sequences of β-Actin orthologs are presented below in Table 1.

Anti-β-Actin antibodies suitable for detecting β-Actin protein are well-known in the art and include, for example, antibody TA353557 (Origene), antibodies NB600-501 and NB600-503 (Novus Biologicals, Littleton, CO), antibodies ab8226 and ab8227 (AbCam, Cambridge, MA), antibody AM4302 (ThermoFisher Scientific), antibody Cat #PM-7669-biotin (ProSci, Poway, CA), etc. In addition, reagents are well-known for detecting $-Actin. Moreover, mutilple siRNA, shRNA, CRISPR constructs for reducing β-Actin expression can be found in the commercial product lists of the above-referenced companies, such as siRNA products #sc-108069 and sc-108070 and CRISPR product #sc-400000-KO-2 from Santa Cruz Biotechnology, RNAi products SR300047 and TL314976V, and CRISPR product KN203643 (Origene), and multiple CRISPR products from GenScript (Piscataway, NJ). It is to be noted that the term can further be used to refer to any combination of features described herein regarding β-Actin molecules. For example, any combination of sequence composition, percentage identify, sequence length, domain structure, functional activity, etc. can be used to describe a β-Actin molecule encompassed by the present invention.

The term “BCL7A” refers to BCL Tumor Suppressor 7A. This gene is directly involved, with Myc and IgH, in a three-way gene translocation in a Burkitt lymphoma cell line. As a result of the gene translocation, the N-terminal region of the gene product is disrupted, which is thought to be related to the pathogenesis of a subset of high-grade B cell non-Hodgkin lymphoma. The N-terminal segment involved in the translocation includes the region that shares a strong sequence similarity with those of BCL7B and BCL7C. Diseases associated with BCL7A include Lymphoma and Burkitt Lymphoma. An important paralog of this gene is BCL7C. Human BCL7A protein has 210 amino acids and a molecular mass of 22810 Da.

The term “BCL7A” is intended to include fragments, variants (e.g., allelic variants), and derivatives thereof. Representative human BCL7A cDNA and human BCL7A protein sequences are well-known in the art and are publicly available from the National Center for Biotechnology Information (NCBI). For example, two different human BCL7A isoforms are known. Human BCL7A isoform a (NP_066273.1) is encodable by the transcript variant 1 (NM_020993.4). Human BCL7A isoform b (NP_001019979.1) is encodable by the transcript variant 2 (NM_001024808.2). Nucleic acid and polypeptide sequences of BCL7A orthologs in organisms other than humans are well known and include, for example, chimpanzee BCL7A (XM_009426452.3 and XP_009424727.2, and XM_016924434.2 and XP_016779923.1), Rhesus monkey BCL7A (XM_015153012.1 and XP_015008498.1, and XM_015153013.1 and XP_015008499.1), dog BCL7A (XM_543381.6 and XP_543381.2, and XM_854760.5 and XP_859853.1), cattle BCL7A (XM_024977701.1 and XP_024833469.1, and XM_024977700.1 and XP_024833468.1), mouse BCL7A (NM_029850.3 and NP_084126.1), rat BCL7A (XM_017598515.1 and XP_017454004.1), chicken BCL7A (XM_004945565.3 and XP_004945622.1, and XM_415148.6 and XP_415148.2), tropical clawed frog BCL7A (NM_001006871.1 and NP_001006872.1), and zebrafish BCL7A (NM_212560.1 and NP_997725.1). Representative sequences of BCL7A orthologs are presented below in Table 1.

Anti-BCL7A antibodies suitable for detecting BCL7A protein are well-known in the art and include, for example, antibody TA344744 (Origene), antibodies NBP1-30941 and NBP1-91696 (Novus Biologicals, Littleton, CO), antibodies ab137362 and ab1075 (AbCam, Cambridge, MA), antibody PA5-27123 (ThermoFisher Scientific), antibody Cat #45-325 (ProSci, Poway, CA), etc. In addition, reagents are well-known for detecting BCL7A. Multiple clinical tests of BCL7A are available in NIH Genetic Testing Registry (GTR®) (e.g., GTR Test ID: GTR000541481.2, offered by Fulgent Clinical Diagnostics Lab (Temple City, CA)). Moreover, mutilple siRNA, shRNA, CRISPR constructs for reducing BCL7A expression can be found in the commercial product lists of the above-referenced companies, such as siRNA products #sc-96136 and sc-141671 and CRISPR product #sc-410702 from Santa Cruz Biotechnology, RNAi products SR300417 and TL314490V, and CRISPR product KN210489 (Origene), and multiple CRISPR products from GenScript (Piscataway, NJ). It is to be noted that the term can further be used to refer to any combination of features described herein regarding BCL7A molecules. For example, any combination of sequence composition, percentage identify, sequence length, domain structure, functional activity, etc. can be used to describe a BCL7A molecule encompassed by the present invention.

The term “BCL7B” refers to BCL Tumor Suppressor 7B, a member of the BCL7 family including BCL7A, BCL7B and BCL7C proteins. This member is BCL7B, which contains a region that is highly similar to the N-terminal segment of BCL7A or BCL7C proteins. The BCL7A protein is encoded by the gene known to be directly involved in a three-way gene translocation in a Burkitt lymphoma cell line. This gene is located at a chromosomal region commonly deleted in Williams syndrome. This gene is highly conserved from C. elegans to human. BCL7B is a positive regulator of apoptosis. BCL7B plays a role in the Wnt signaling pathway, negatively regulating the expression of Wnt signaling components CTNNB1 and HMGA1 (Uehara et al. (2015) PLoS Genet 11(1):e1004921). BCL7B is involved in cell cycle progression, maintenance of the nuclear structure and stem cell differentiation (Uehara et al. (2015) PLoS Genet 11(1):e1004921). It plays a role in lung tumor development or progression. Human BCL7B protein has 202 amino acids and a molecular mass of 22195 Da.

The term “BCL7B” is intended to include fragments, variants (e.g., allelic variants), and derivatives thereof. Representative human BCL7B cDNA and human BCL7B protein sequences are well-known in the art and are publicly available from the National Center for Biotechnology Information (NCBI). For example, three different human BCL7B isoforms are known. Human BCL7B isoform 1 (NP_001698.2) is encodable by the transcript variant 1 (NM_001707.3). Human BCL7B isoform 2 (NP_001184173.1) is encodable by the transcript variant 2 (NM_001197244.1). Human BCL7B isoform 3 (NP_001287990.1) is encodable by the transcript variant 3 (NM_001301061.1). Nucleic acid and polypeptide sequences of BCL7B orthologs in organisms other than humans are well known and include, for example, chimpanzee BCL7B (XM_003318671.3 and XP_003318719.1, and XM_003318672.3 and XP_003318720.1), Rhesus monkey BCL7B (NM_001194509.1 and NP_001181438.1), dog BCL7B (XM_546926.6 and XP_546926.1, and XM_005620975.2 and XP_005621032.1), cattle BCL7B (NM_001034775.2 and NP_001029947.1), mouse BCL7B (NM_009745.2 and NP_033875.2), chicken BCL7B (XM_003643231.4 and XP_003643279.1, XM_004949975.3 and XP_004950032.1, and XM_025142155.1 and XP_024997923.1), tropical clawed frog BCL7B (NM_001103072.1 and NP_001096542.1), and zebrafish BCL7B (NM_001006018.1 and NP_001006018.1, and NM_213165.1 and NP_998330.1). Representative sequences of BCL7B orthologs are presented below in Table 1.

Anti-BCL7B antibodies suitable for detecting BCL7B protein are well-known in the art and include, for example, antibody TA809485 (Origene), antibodies H00009275-MO1 and NBP2-34097 (Novus Biologicals, Littleton, CO), antibodies ab130538 and ab172358 (AbCam, Cambridge, MA), antibody MA527163 (ThermoFisher Scientific), antibody Cat #58-996 (ProSci, Poway, CA), etc. In addition, reagents are well-known for detecting BCL7B. Moreover, mutilple siRNA, shRNA, CRISPR constructs for reducing BCL7B expression can be found in the commercial product lists of the above-referenced companies, such as siRNA products #sc-89728 and sc-141672 and CRISPR product #sc-411262 from Santa Cruz Biotechnology, RNAi products SR306141 and TL306418V, and CRISPR product KN201696 (Origene), and multiple CRISPR products from GenScript (Piscataway, NJ). It is to be noted that the term can further be used to refer to any combination of features described herein regarding BCL7B molecules. For example, any combination of sequence composition, percentage identify, sequence length, domain structure, functional activity, etc. can be used to describe a BCL7B molecule encompassed by the present invention.

The term “BCL7C” refers to BCL Tumor Suppressor 7C, a member of the BCL7 family including BCL7A, BCL7B and BCL7C proteins. This gene is identified by the similarity of its product to the N-terminal region of BCL7A protein. BCL7C may play an anti-apoptotic role. Diseases associated with BCL7C include Lymphoma. Human BCL7C protein has 217 amino acids and a molecular mass of 23468 Da.

The term “BCL7C” is intended to include fragments, variants (e.g., allelic variants), and derivatives thereof. Representative human BCL7C cDNA and human BCL7C protein sequences are well-known in the art and are publicly available from the National Center for Biotechnology Information (NCBI). For example, two different human BCL7C isoforms are known. Human BCL7C isoform 1 (NP_001273455.1) is encodable by the transcript variant 1 (NM_001286526.1). Human BCL7C isoform 2 (NP_004756.2) is encodable by the transcript variant 2 (NM_004765.3). Nucleic acid and polypeptide sequences of BCL7C orthologs in organisms other than humans are well known and include, for example, chimpanzee BCL7C (XM_016929717.2 and XP_016785206.1, XM_016929716.2 and XP_016785205.1, and XM_016929718.2 and XP_016785207.1), Rhesus monkey BCL7C (NM_001265776.2 and NP_001252705.1), cattle BCL7C (NM_001099722.1 and NP_001093192.1), mouse BCL7C (NM_001347652.1 and NP_001334581.1, and NM_009746.2 and NP_033876.1), and rat BCL7C (NM_001106298.1 and NP_001099768.1). Representative sequences of BCL7C orthologs are presented below in Table 1.

Anti-BCL7C antibodies suitable for detecting BCL7C protein are well-known in the art and include, for example, antibody TA347083 (Origene), antibodies NBP2-15559 and NBP1-86441 (Novus Biologicals, Littleton, CO), antibodies ab126944 and ab231278 (AbCam, Cambridge, MA), antibody PA5-30308 (ThermoFisher Scientific), etc. In addition, reagents are well-known for detecting BCL7C. Multiple clinical tests of BCL7C are available in NIH Genetic Testing Registry (GTR®) (e.g., GTR Test ID: GTR000540637.2, offered by Fulgent Clinical Diagnostics Lab (Temple City, CA)). Moreover, mutilple siRNA, shRNA, CRISPR constructs for reducing BCL7C expression can be found in the commercial product lists of the above-referenced companies, such as siRNA products #sc-93022 and sc-141673 and CRISPR product #sc-411261 from Santa Cruz Biotechnology, RNAi products SR306140 and TL315552V, and CRISPR product KN205720 (Origene), and multiple CRISPR products from GenScript (Piscataway, NJ). It is to be noted that the term can further be used to refer to any combination of features described herein regarding BCL7C molecules. For example, any combination of sequence composition, percentage identify, sequence length, domain structure, functional activity, etc. can be used to describe a BCL7C molecule encompassed by the present invention.

The term “SMARCA4” refers to SWI/SNF related, matrix associated, actin dependent regulator of chromatin, subfamily a, member 4, a member of the SWI/SNF family of proteins and is highly similar to the brahma protein of Drosophila. Members of this family have helicase and ATPase activities and are thought to regulate transcription of certain genes by altering the chromatin structure around those genes. The encoded protein is part of the large ATP-dependent chromatin remodeling complex SNF/SWI, which is required for transcriptional activation of genes normally repressed by chromatin. In addition, this protein can bind BRCA1, as well as regulate the expression of the tumorigenic protein CD44. Mutations in this gene cause rhabdoid tumor predisposition syndrome type 2. SMARCA4 is a component of SWI/SNF chromatin remodeling complexes that carry out key enzymatic activities, changing chromatin structure by altering DNA-histone contacts within a nucleosome in an ATP-dependent manner. SMARCA4 is a component of the CREST-BRG1 complex, a multiprotein complex that regulates promoter activation by orchestrating a calcium-dependent release of a repressor complex and a recruitment of an activator complex. In resting neurons, transcription of the c-FOS promoter is inhibited by BRG1-dependent recruitment of a phospho-RB1-HDAC repressor complex. Upon calcium influx, RB1 is dephosphorylated by calcineurin, which leads to release of the repressor complex. At the same time, there is increased recruitment of CREBBP to the promoter by a CREST-dependent mechanism, which leads to transcriptional activation. The CREST-BRG1 complex also binds to the NR2B promoter, and activity-dependent induction of NR2B expression involves a release of HDAC1 and recruitment of CREBBP. SMARCA4 belongs to the neural progenitors-specific chromatin remodeling complex (npBAF complex) and the neuron-specific chromatin remodeling complex (nBAF complex). During neural development a switch from a stem/progenitor to a postmitotic chromatin remodeling mechanism occurs as neurons exit the cell cycle and become committed to their adult state. The transition from proliferating neural stem/progenitor cells to postmitotic neurons requires a switch in subunit composition of the npBAF and nBAF complexes. As neural progenitors exit mitosis and differentiate into neurons, npBAF complexes which contain ACTL6A/BAF53A and PHF10/BAF45A, are exchanged for homologous alternative ACTL6B/BAF53B and DPF1/BAF45B or DPF3/BAF45C subunits in neuron-specific complexes (nBAF). The npBAF complex is essential for the self-renewal/proliferative capacity of the multipotent neural stem cells. The nBAF complex along with CREST plays a role regulating the activity of genes essential for dendrite growth. SMARCA4/BAF190A promote neural stem cell self-renewal/proliferation by enhancing Notch-dependent proliferative signals, while concurrently making the neural stem cell insensitive to SHH-dependent differentiating cues. SMARCA4 acts as a corepressor of ZEB1 to regulate E-cadherin transcription and is required for induction of epithelial-mesenchymal transition (EMT) by ZEB1. Human SMARCA4 protein has 1647 amino acids and a molecular mass of 184646 Da. The known binding partners of SMARCA4 include, e.g., PHF10/BAF45A, MYOG, IKFZ1, ZEB1, NR3C1, PGR, SMARD1, TOPBP1 and ZMIM2/ZIMP7.

The term “SMARCA4” is intended to include fragments, variants (e.g., allelic variants), and derivatives thereof. Representative human SMARCA4 cDNA and human SMARCA4 protein sequences are well-known in the art and are publicly available from the National Center for Biotechnology Information (NCBI). For example, six different human SMARCA4 isoforms are known. Human SMARCA4 isoform A (NP_001122321.1) is encodable by the transcript variant 1 (NM_001128849.1). Human SMARCA4 isoform B (NP_001122316.1 and NP_003063.2) is encodable by the transcript variant 2 (NM_001128844.1) and the transcript variant 3 (NM_003072.3). Human SMARCA4 isoform C (NP_001122317.1) is encodable by the transcript variant 4 (NM_001128845.1). Human SMARCA4 isoform D (NP_001122318.1) is encodable by the transcript variant 5 (NM_001128846.1). Human SMARCA4 isoform E (NP_001122319.1) is encodable by the transcript variant 6 (NM_001128847.1). Human SMARCA4 isoform F (NP_001122320.1) is encodable by the transcript variant 7 (NM_001128848.1). Nucleic acid and polypeptide sequences of SMARCA4 orthologs in organisms other than humans are well known and include, for example, Rhesus monkey SMARCA4 (XM_015122901.1 and XP_014978387.1, XM_015122902.1 and XP_014978388.1, XM_015122903.1 and XP_014978389.1, XM_015122906.1 and XP_014978392.1, XM_015122905.1 and XP_014978391.1, XM_015122904.1 and XP_014978390.1, XM_015122907.1 and XP_014978393.1, XM_015122909.1 and XP_014978395.1, and XM_015122910.1 and XP_014978396.1), cattle SMARCA4 (NM_001105614.1 and NP_001099084.1), mouse SMARCA4 (NM_001174078.1 and NP_001167549.1, NM 011417.3 and NP_035547.2, NM_001174079.1 and NP_001167550.1, NM_001357764.1 and NP_001344693.1), rat SMARCA4 (NM_134368.1 and NP_599195.1), chicken SMARCA4 (NM_205059.1 and NP_990390.1), and zebrafish SMARCA4 (NM_181603.1 and NP_853634.1). Representative sequences of SMARCA4 orthologs are presented below in Table 1.

Anti-SMARCA4 antibodies suitable for detecting SMARCA4 protein are well-known in the art and include, for example, antibody AM26021PU-N(Origene), antibodies NB100-2594 and AF5738 (Novus Biologicals, Littleton, CO), antibodies ab110641 and ab4081 (AbCam, Cambridge, MA), antibody 720129 (ThermoFisher Scientific), antibody 7749 (ProSci), etc. In addition, reagents are well-known for detecting SMARCA4. Multiple clinical tests of SMARCA4 are available in NIH Genetic Testing Registry (GTR®) (e.g., GTR Test ID: GTR000517106.2, offered by Fulgent Clinical Diagnostics Lab (Temple City, CA)). Moreover, mutilple siRNA, shRNA, CRISPR constructs for reducing SMARCA4 expression can be found in the commercial product lists of the above-referenced companies, such as siRNA products #sc-29827 and sc-44287 and CRISPR product #sc-400168 from Santa Cruz Biotechnology, RNAi products SR321835 and TL309249V, and CRISPR product KN219258 (Origene), and multiple CRISPR products from GenScript (Piscataway, NJ). It is to be noted that the term can further be used to refer to any combination of features described herein regarding SMARCA4 molecules. For example, any combination of sequence composition, percentage identify, sequence length, domain structure, functional activity, etc. can be used to describe a SMARCA4 molecule encompassed by the present invention.

The term “SS18” refers to SS18, NBAF Chromatin Remodeling Complex Subunit. SS18 functions synergistically with RBM14 as a transcriptional coactivator. Isoform 1 and isoform 2 of SS18 function in nuclear receptor coactivation. Isoform 1 and isoform 2 of SS18 function in general transcriptional coactivation. Diseases associated with SS18 include Sarcoma, Synovial Cell Sarcoma. Among its related pathways are transcriptional misregulation in cancer and chromatin regulation/acetylation. Human SS18 protein has 418 amino acids and a molecular mass of 45929 Da. The known binding partners of SS18 include, e.g., MLLT10 and RBM14 isoform 1.

The term “SS18” is intended to include fragments, variants (e.g., allelic variants), and derivatives thereof. Representative human SS18 cDNA and human SS18 protein sequences are well-known in the art and are publicly available from the National Center for Biotechnology Information (NCBI). For example, three different human SS18 isoforms are known. Human SS18 isoform 1 (NP_001007560.1) is encodable by the transcript variant 1 (NM_001007559.2). Human SS18 isoform 2 (NP_005628.2) is encodable by the transcript variant 2 (NM_005637.3). Human SS18 isoform 3 (NP_001295130.1) is encodable by the transcript variant 3 (NM_001308201.1). Nucleic acid and polypeptide sequences of SS18 orthologs in organisms other than humans are well known and include, for example, dog SS18 (XM_005622940.3 and XP_005622997.1, XM_537295.6 and XP_537295.3, XM_003434925.4 and XP_003434973.1, and XM_005622941.3 and XP_005622998.1), mouse SS18 (NM_009280.2 and NP_033306.2, NM_001161369.1 and NP_001154841.1, NM_001161370.1 and NP_001154842.1, and NM_001161371.1 and NP_001154843.1), rat SS18 (NM_001100900.1 and NP_001094370.1), chicken SS18 (XM_015277943.2 and XP_015133429.1, and XM_015277944.2 and XP_015133430.1), tropical clawed frog SS18 (XM_012964966.1 and XP_012820420.1, XM_018094711.1 and XP_017950200.1, XM_012964964.2 and XP_012820418.1, and XM_012964965.2 and XP_012820419.1), and zebrafish SS18 (NM_001291325.1 and NP_001278254.1, and NM_199744.2 and NP_956038.1). Representative sequences of BRD7 orthologs are presented below in Table 1.

Anti-SS18 antibodies suitable for detecting SS18 protein are well-known in the art and include, for example, antibody TA314572 (Origene), antibodies NBP2-31777 and NBP2-31612 (Novus Biologicals, Littleton, CO), antibodies ab179927 and ab89086 (AbCam, Cambridge, MA), antibody PA5-63745 (ThermoFisher Scientific), etc. In addition, reagents are well-known for detecting SS18. Multiple clinical tests of SS18 are available in NIH Genetic Testing Registry (GTR®) (e.g., GTR Test ID: GTR000546059.2, offered by Fulgent Clinical Diagnostics Lab (Temple City, CA)). Moreover, mutilple siRNA, shRNA, CRISPR constructs for reducing SS18 expression can be found in the commercial product lists of the above-referenced companies, such as siRNA products #sc-38449 and sc-38450 and CRISPR product #sc-401575 from Santa Cruz Biotechnology, RNAi products SR304614 and TL309102V, and CRISPR product KN215192 (Origene), and multiple CRISPR products from GenScript (Piscataway, NJ). It is to be noted that the term can further be used to refer to any combination of features described herein regarding SS18 molecules. For example, any combination of sequence composition, percentage identify, sequence length, domain structure, functional activity, etc. can be used to describe a SS18 molecule encompassed by the present invention.

The term “SSX” refers to a family of highly homologous synovial sarcoma X (SSX) breakpoint proteins. The mammalian SSX family proteins include, e.g., human SSX1-9. These proteins can function as transcriptional repressors. They are also capable of eliciting spontaneous humoral and cellular immune responses in cancer patients, and are useful targets in cancer vaccine-based immunotherapy. SSX1, SSX2 and SSX4 family members, have been involved in t(X;18)(p11.2;q11.2) translocations that are characteristically found in all synovial sarcomas. This translocation results in the fusion of the synovial sarcoma translocation gene on chromosome 18 to one of the SSX genes on chromosome X. The encoded hybrid proteins are responsible for transforming activity. While some of the related SSX genes are involved in t(X;18)(p11.2;q11.2) translocations that are characteristically found in all synovial sarcomas, SSX3, SSX5, and SSX7 do not appear to be involved in such translocations. SSX6, or SSX6P is classified as a pseudogene because a splice donor in the 3′ UTR has changed compared to other family numbers, rendering the transcript a candidate for nonsense-mediated mRNA decay (NMD). SSX8, or SSX8P (SSX Family Member 8, Pseudogene) is a Pseudogene. SSX9, or SSX9P (SSX Family Member 9, Pseudogene) is a Pseudogene. SSX C-terminus comprises a 6-amino acid basic region and a 7-amino adic acidic region. The. representative basic regions and acidic regions for SSX1 to SSX9 are shown in FIG. 3D.

The term “SSX1” is intended to include fragments, variants (e.g., allelic variants), and derivatives thereof. Representative human SSX1 cDNA and human SSX1 protein sequences are well-known in the art and are publicly available from the National Center for Biotechnology Information (NCBI). For example, two different human SSX1 transcript variants are known. Human transcript variant 1 (NM_001278691.2) and human transcript variant 2 (NM_005635.4) encode the same human SSX1 protein (NP_001265620.1 and NP_005626.1). Transcript variant 1 represents the longer transcript. Transcript variant 2 differs in the 5′ UTR compared to variant 1. Nucleic acid and polypeptide sequences of SSX1 orthologs in organisms other than humans are well known and include, for example, monkey SS18 (XM_017854812.1 and XP_017710301.1), and chimpanzee SS18 (XM_016944028.1 and XP_016799517.1, XM_016944029.1 and XP_016799518.1, XM_016944031.1 and XP_016799520.1, and XM_016944030.1 and XP_016799519.1). A representative SSX1 has 188 amino acids with a molecular mass of 21931 Da. Representative sequences of SSX1 orthologs are presented below in Table 1.

Anti-SSX1 antibodies suitable for detecting SSX1 protein are well-known in the art and include, for example, antibodies CF502523 and CF502693 (Origene), antibodies NBP2-00614 and H00006756-MO1 (Novus Biologicals, Littleton, CO), antibodies ab206839 and ab234815 (AbCam, Cambridge, MA), antibody MA5-25511 (ThermoFisher Scientific), etc. In addition, reagents are well-known for detecting SSX1. Moreover, mutilple siRNA, shRNA, CRISPR constructs for reducing SSX1 expression can be found in the commercial product lists of the above-referenced companies, such as siRNA products #sc-44120 and sc-44120-SH and CRISPR product #sc-403551 from Santa Cruz Biotechnology, RNAi products SR304610 and TL309084, and CRISPR product KN401600 (Origene), and multiple CRISPR products from GenScript (Piscataway, NJ). It is to be noted that the term can further be used to refer to any combination of features described herein regarding SSX1 molecules. For example, any combination of sequence composition, percentage identify, sequence length, domain structure, functional activity, etc. can be used to describe a SSX1 molecule encompassed by the present invention.

The term “SSX2” is intended to include fragments, variants (e.g., allelic variants), and derivatives thereof. Representative human SSX2 cDNA and human SSX2 protein sequences are well-known in the art and are publicly available from the National Center for Biotechnology Information (NCBI). For example, three different human SSX2 transcript variants are known. Human SSX2 isoform 1 (NP_003138.3) is encodable by the transcript variant 1 (NM_003147.5). Human SSX2 isoform 2 (NP_783629.1) is encodable by the transcript variant 2 (NM_175698.2). Human SSX2 isoform 3 (NP_001265626.1) is encodable by the transcript variant 3 (NM_001278697.1). SSX2 has an identical duplicate, SSX2B (GeneID: 727837), located about 45 kb downstream in the opposite orientation on chromosome X. Three different human SSX2B transcript variants are known. Human SSX2B isoform 1 (NP_001265630.1) is encodable by the transcript variant 1 (NM_001278701.2). Human SSX2B isoform 2 (NP_001157889.1) is encodable by the transcript variant 2 (NM_001164417.3). Human SSX2B isoform 3 (NP_001265631.1) is encodable by the transcript variant 3 (NM_001278702.2). Nucleic acid and polypeptide sequences of SSX2 orthologs in organisms other than humans are well known. Representative sequences of SSX2 orthologs are presented below in Table 1.

Anti-SSX2 antibodies suitable for detecting SSX2 protein are well-known in the art and include, for example, antibodies CF500618 and CF500620 (Origene), antibodies NBP1-48008 and H00006757-MO1 (Novus Biologicals, Littleton, CO), antibodies ab236415 and ab48571 (AbCam, Cambridge, MA), antibody MA5-24971 (ThermoFisher Scientific), etc. In addition, reagents are well-known for detecting SSX2. Moreover, mutilple siRNA, shRNA, CRISPR constructs for reducing SSX2 expression can be found in the commercial product lists of the above-referenced companies, such as siRNA product #sc-38446 and CRISPR product #sc-417124 from Santa Cruz Biotechnology, RNAi products SR304611 and TL309083, and CRISPR product KN401214 (Origene), and multiple CRISPR products from GenScript (Piscataway, NJ). It is to be noted that the term can further be used to refer to any combination of features described herein regarding SSX2 molecules. For example, any combination of sequence composition, percentage identify, sequence length, domain structure, functional activity, etc. can be used to describe a SSX2 molecule encompassed by the present invention.

The term “SSX4” is intended to include fragments, variants (e.g., allelic variants), and derivatives thereof. Representative human SSX4 cDNA and human SSX4 protein sequences are well-known in the art and are publicly available from the National Center for Biotechnology Information (NCBI). For example, two different human SSX4 transcript variants are known. Human SSX4 isoform 1 (NP_005627.1) is encodable by the transcript variant 1 (NM_005636.4). Human SSX4 isoform 2 (NP_783856.1) is encodable by the transcript variant 2 (NM_175729.1). Chromosome Xp11 contains a segmental duplication resulting in two identical copies of synovial sarcoma, X breakpoint 4, SSX4 and SSX4B, in tail-to-tail orientation. Two different human SSX4B transcript variants are known. Human SSX4B isoform a (NP_001030004.1) is encodable by the transcript variant 1 (NM_001034832.3). Human SSX4B isoform 2 (NP_001035702.1) is encodable by the transcript variant 2 (NM_001040612.2). Nucleic acid and polypeptide sequences of SSX4 orthologs in organisms other than humans are well known, for example, dog putative protein SSX6-like (XM_005641306.2 and XP_005641363.1 and XM_022416309.1 and XP_022272017.1), cattle protein SSX1-like (XM_024988534.1 and XP_024844302.1), cattle synovial sarcoma, X breakpoint 5 (XM_024988283.1 and XP_024844051.1, and XM_024988284.1 and XP_024844052.1), and mouse synovial sarcoma, X member B, breakpoint 2 (NM_001001450.4 and NP_001001450.1, and NM_001134226.1 and NP_001127698.1). Representative sequences of SSX4 orthologs are presented below in Table 1.

Anti-SSX4 antibodies suitable for detecting SSX4 protein are well-known in the art and include, for example, antibodies TA339114 and TA339115 (Origene), antibodies H00006759-M02 and H00006759-B01P (Novus Biologicals, Littleton, CO), antibody ab172215 (AbCam, Cambridge, MA), antibody PA5-41117 (ThermoFisher Scientific), etc. In addition, reagents are well-known for detecting SSX4. Moreover, mutilple siRNA, shRNA, CRISPR constructs for reducing SSX4 expression can be found in the commercial product lists of the above-referenced companies, such as siRNA products #sc-106732 and sc-106800 and CRISPR product #sc-416410 from Santa Cruz Biotechnology, RNAi products SR304613 and TL309081, and CRISPR product KN422659 (Origene), and multiple CRISPR products from GenScript (Piscataway, NJ). It is to be noted that the term can further be used to refer to any combination of features described herein regarding SSX4 molecules. For example, any combination of sequence composition, percentage identify, sequence length, domain structure, functional activity, etc. can be used to describe a SSX4 molecule encompassed by the present invention.

The term “SSX3” is intended to include fragments, variants (e.g., allelic variants), and derivatives thereof. Representative human SSX3 cDNA and human SSX3 protein sequences are well-known in the art and are publicly available from the National Center for Biotechnology Information (NCBI). For example, Human SSX3 (NP_066294.1) is encodable by the transcript (NM_021014.4). Nucleic acid and polypeptide sequences of SSX3 orthologs in organisms other than humans are well known, for example, monkey SSX3 (XM_002806224.3 and XP_002806270.1). Representative sequences of SSX3 orthologs are presented below in Table 1.

Anti-SSX3 antibodies suitable for detecting SSX3 protein are well-known in the art and include, for example, antibody TA345316 (Origene), antibodies H00010214-M03 and H00010214-B01P (Novus Biologicals, Littleton, CO), antibody ab160884 (AbCam, Cambridge, MA), antibodies MA5-24431 and PA5-69016 (ThermoFisher Scientific), etc. In addition, reagents are well-known for detecting SSX3. Moreover, mutilple siRNA, shRNA, CRISPR constructs for reducing SSX3 expression can be found in the commercial product lists of the above-referenced companies, such as siRNA products #sc-38447 and sc-38447-SH and CRISPR product #sc-417585 from Santa Cruz Biotechnology, RNAi products SR306902 and TL301375, and CRISPR product KN403244 (Origene), and multiple CRISPR products from GenScript (Piscataway, NJ). It is to be noted that the term can further be used to refer to any combination of features described herein regarding SSX3 molecules. For example, any combination of sequence composition, percentage identify, sequence length, domain structure, functional activity, etc. can be used to describe a SSX3 molecule encompassed by the present invention.

The term “SSX5” is intended to include fragments, variants (e.g., allelic variants), and derivatives thereof. Representative human SSX5 cDNA and human SSX5 protein sequences are well-known in the art and are publicly available from the National Center for Biotechnology Information (NCBI). For example, two different human SSX5 transcript variants are known. Human SSX5 isoform 1 (NP_066295.3) is encodable by the transcript variant 1 (NM_021015.4). Human SSX5 isoform 2 (NP_783729.1) is encodable by the transcript variant 2 (NM_175723.1). Nucleic acid and polypeptide sequences of SSX5 orthologs in organisms other than humans are well known. Representative sequences of SSX5 orthologs are presented below in Table 1.

Anti-SSX5 antibodies suitable for detecting SSX5 protein are well-known in the art and include, for example, antibodies CF504221 and CF504223 (Origene), antibodies NBP2-01842 and H00006758-B01P (Novus Biologicals, Littleton, CO), antibodies PA5-92141 and MA5-25901 (ThermoFisher Scientific), etc. In addition, reagents are well-known for detecting SSX5. Moreover, mutilple siRNA, shRNA, CRISPR constructs for reducing SSX5 expression can be found in the commercial product lists of the above-referenced companies, such as siRNA products #sc-38448 and sc-38448-SH and CRISPR product #sc-403552 from Santa Cruz Biotechnology, RNAi products SR304612 and TL301374, and CRISPR product KN402208 (Origene), and multiple CRISPR products from GenScript (Piscataway, NJ). It is to be noted that the term can further be used to refer to any combination of features described herein regarding SSX5 molecules. For example, any combination of sequence composition, percentage identify, sequence length, domain structure, functional activity, etc. can be used to describe a SSX5 molecule encompassed by the present invention.

The term “SSX7” is intended to include fragments, variants (e.g., allelic variants), and derivatives thereof. Representative human SSX7 cDNA and human SSX7 protein sequences are well-known in the art and are publicly available from the National Center for Biotechnology Information (NCBI). For example, Human SSX7 (NP_775494.1) is encodable by the transcript (NM_173358.2). Nucleic acid and polypeptide sequences of SSX7 orthologs in organisms other than humans are well known. Representative sequences of SSX7 orthologs are presented below in Table 1.

Anti-SSX7 antibodies suitable for detecting SSX7 protein are well-known in the art and include, for example, antibody TA339916 (Origene), antibody NBP1-79468 (Novus Biologicals, Littleton, CO), antibody PA5-49262 (ThermoFisher Scientific), etc. In addition, reagents are well-known for detecting SSX7. Moreover, mutilple siRNA, shRNA, CRISPR constructs for reducing SSX7 expression can be found in the commercial product lists of the above-referenced companies, such as siRNA products #sc-106568 and sc-106568-SH and CRISPR product #sc-403553 from Santa Cruz Biotechnology, RNAi products SR316959 and TL301372, and CRISPR product KN413920 (Origene), and multiple CRISPR products from GenScript (Piscataway, NJ). It is to be noted that the term can further be used to refer to any combination of features described herein regarding SSX7 molecules. For example, any combination of sequence composition, percentage identify, sequence length, domain structure, functional activity, etc. can be used to describe a SSX7 molecule encompassed by the present invention.

The SS18-SSX fusion protein is formed by chromosomal translocation, which results in a fusion of SS18 protein with the C-terminal of the SSX family member (e.g., SSX1, SSX2, and SSX4). Many of these function as oncoproteins which play important roles in tumorgenesis. For example, the molecular hallmark of synovial sarcoma is a pathognomonic reciprocal translocation t(X;18)(p11; q11), leading to the fusion of SS18 (SYT) to one of the homologs SSX genes (most frequently SSX1 or SSX2, in rare cases SSX4), generating oncogenic SS18-SSX chimeric proteins. Representative sequences of SS18-SSX fusion proteins are presented below in Table 2.

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CHi domains; (ii) a F(ab′)₂ fragment, a bivalent fragment comprising two Fab fragments linked by a disulfide bridge at the hinge region; (iii) a Fd fragment consisting of the VH and CHi domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies encompassed by the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

The term “biomarker” refers to a measurable entity of the present invention that has been determined to be predictive of cancer therapy effects (e.g., SS18-SSX target genes described described herein, such as those in the tables, figures, examples, and otherwise described in the specification). Biomarkers can include, without limitation, nucleic acids (e.g., genomic nucleic acids and/or transcribed nucleic acids) and proteins. Many biomarkers are also useful as therapeutic targets.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of SS18-SSX oncogenic fusion protein target genes.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In certain embodiments, the cancer encompasses synovial sarcoma. Synovial sarcoma is an aggressive malignancy comprising 7-10% of all soft tissue tumors with a predominance in adolescents and young adults. The molecular hallmark of synovial sarcoma is a pathognomonic reciprocal translocation t(X;18)(p11; q11), leading to the fusion of SS18 (SYT) to one of the homologs SSX genes (most frequently SSX1 or SSX2, in rare cases SSX4), generating oncogenic SS18-SSX chimeric proteins.

Synovial sarcoma is a rare cancer. Only about 1 to 3 individuals in a million people are diagnosed with this disease each year. The diagnosis starts with imaging studies. X-ray, sonogram, CT scan, and MRI may be used in the course of evaluating a suspicious mass. After imaging studies, the next step in diagnosis is a biopsy to remove a sample of the tumor for further analysis. Among the different types of biopsies, open biopsy (a surgical incision is made to remove the sample) or core needle biopsy (a large needle is used to take the sample) are preferred. Normally, the sample tissue obtained from the biopsy is sent directly from the procedure room to a pathology laboratory to be sliced and fixed on small glass plates (slides). The pathologist commonly uses a technique called immunohistochemistry to learn about the tumor cells. Another technique called cytogenetics is often used to detect the chromosomal translocation specific to synovial sarcoma, which helps to confirm the diagnosis. Once a tumor has been deemed malignant, further imaging studies such as a PET scan of the whole body and/or CT scan of the chest, abdomen or pelvis may be used to look for possible metastases.

The primary treatment for synovial sarcoma is surgery to remove the entire tumor with clear margins when possible. “Clear margins” are achieved when healthy tissue surrounding the tumor is removed along with the tumor, making it more likely that all cancer cells have been removed from the area. Depending on the location and size of the mass, it may be difficult for a surgeon to remove adequate margins around the tumor while preserving function. Radiotherapy may also be used, either before or after surgery, to reduce the risk of leaving cells behind. Chemotherapy (typically Doxorubicin and/or Ifosfamide) may be recommended in the treatment of synovial sarcoma, especially in advanced or metastatic disease.

Prognosis in synovial sarcoma patients is influenced by the quality of surgery patients receive and the characteristics of the disease (including tumor size, local invasiveness, histological subtype, presence of metastases, and lymph node involvement). Patients with small tumors that can be completely removed with adequate margins at diagnosis have an excellent prognosis. The risk of developing distant metastases is higher for patients with tumors that are larger than 5 cm. Patients with the poorly differentiated subtype are considered to have a worse prognosis than those with other subtypes, and patients with metastases that cannot be removed have a poor prognosis.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods encompassed by the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Conventional T cells, also known as Tconv or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naive T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naive Tcons” are CD4⁺ T cells that have differentiated in bone marrow, and successfully underwent a positive and negative processes of central selection in a thymus, but have not yet been activated by exposure to an antigen. Naive Tcons are commonly characterized by surface expression of L-selectin (CD62L), absence of activation markers such as CD25, CD44 or CD69, and absence of memory markers such as CD45RO. Naive Tcons are therefore believed to be quiescent and non-dividing, requiring interleukin-7 (IL-7) and interleukin-15 (IL- 15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, immunotherapy comprises inhibitors of one or more immune checkpoints. The term “immune checkpoint” refers to a group of molecules on the cell surface of CD4+ and/or CD8+T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two R sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of R strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.I. PD-L1 proteins comprise a signal sequence, and an IgV domain and an IgC domain. The signal sequence of PD-L1S is from about amino acid 1 to about amino acid 18. The signal sequence of PD-L1M is from about amino acid 1 to about amino acid 18. The IgV domain of PD-L1S is from about amino acid 19 to about amino acid 134 and the IgV domain of PD-L1M is from about amino acid 19 to about amino acid 134. The IgC domain of PD-L1S is from about amino acid 135 to about amino acid 227 and the IgC domain of PD-L1M is from about amino acid 135 to about amino acid 227. The hydrophilic tail of the PD-L1 exemplified in PD-L1S comprises a hydrophilic tail shown from about amino acid 228 to about amino acid 245. The PD-L1 polypeptide of PD-L1M comprises a transmembrane domain from about amino acids 239 to about amino acid 259 of PD-L1M and a cytoplasmic domain shown from about amino acid 260 to about amino acid 290 of PD-L1M. In addition, nucleic acid and polypeptide sequences of PD-L1 orthologs in organisms other than humans are well-known and include, for example, rat PD-L1 (NM_001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well-known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. For example, amino acids 1-19 of PD-L2 comprises a signal sequence. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19: 235-263. In still another embodiment, amino acid residues 20-120 of the native human PD-L2 polypeptide and amino acid residues 1-101 of the mature polypeptide comprise an IgV domain. Amino acid residues 121-219 of the native human PD-L2 polypeptide and amino acid residues 102-200 of the mature polypeptide comprise an IgC domain. As used herein, IgV and IgC domains are recognized in the art as Ig superfamily member domains. These domains correspond to structural units that have distinct folding patterns called Ig folds. Ig folds are comprised of a sandwich of two B sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the Cl set within the Ig superfamily. Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well-known and include, for example, rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-L2 polypeptide with its natural binding partner (i.e., physiologically relevant interacting macromolecule involved in an immune function or other biologically relevant function), e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra- or intercellular signaling, 3) modulate activation of immune cells, e.g., T lymphocytes, and 4) modulate the immune response of an organism, e.g., a human organism.

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes decreasing, reducing, limiting, and/or blocking, of, for example a particular action, function, and/or interaction. In some embodiments, the interation between two molecules is “inhibited” if the interaction is reduced, blocked, disrupted or destabilized.

In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “predictive” includes the use of a biomarker nucleic acid and/or protein status, e.g., over- or under- activity, emergence, expression, growth, remission, recurrence or resistance of tumors before, during or after therapy, for determining the likelihood of response of a cancer to an agent that inhibits binding of a SS18-SSX fusion protein to an H2A K119Ub-marked nucleosome alone or in combination with an immunotherapy and/or cancer therapy. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to an agent that inhibits binding of a SS18-SSX fusion protein to an H2A K119Ub-marked nucleosome alone or in combination with an immunotherapy and/or cancer therapy, or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “cancer response,” “response to immunotherapy,” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi). “RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as bone marrow and bone sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10-7 M, such as approximately less than 10⁻⁸ M, 10⁻⁹ μM or 10⁻¹⁰ μM or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more cancer agents (e.g., an agent that inhibits binding of a SS18-SSX fusion protein with a H2AK119Ub-marked nucleosome in combination with immunotherapy) can be greater than the sum of the separate effects of the cancer agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the API sequence that can be found within the enhancer (Kang et al. (1992) Science 257:1134).

As used herein, the term “protein complex” means a composite unit that is a combination of two or more proteins formed by interaction between the proteins. Typically, but not necessarily, a “protein complex” is formed by the binding of two or more proteins together through specific non-covalent binding interactions. However, covalent bonds may also be present between the interacting partners. For instance, the two interacting partners can be covalently crosslinked so that the protein complex becomes more stable. The protein complex may or may not include and/or be associated with other molecules such as nucleic acid, such as RNA or DNA, or lipids or further cofactors or moieties selected from a metal ions, hormones, second messengers, phosphate, sugars. A “protein complex” encompassed by the present invention may also be part of or a unit of a larger physiological protein assembly.

The term “isolated protein complex” means a protein complex present in a composition or environment that is different from that found in nature, in its native or original cellular or body environment. Preferably, an “isolated protein complex” is separated from at least 50%, more preferably at least 75%, most preferably at least 90% of other naturally co-existing cellular or tissue components. Thus, an “isolated protein complex” may also be a naturally existing protein complex in an artificial preparation or a non-native host cell. An “isolated protein complex” may also be a “purified protein complex”, that is, a substantially purified form in a substantially homogenous preparation substantially free of other cellular components, other polypeptides, viral materials, or culture medium, or, when the protein components in the protein complex are chemically synthesized, free of chemical precursors or by-products associated with the chemical synthesis. A “purified protein complex” typically means a preparation containing preferably at least 75%, more preferably at least 85%, and most preferably at least 95% of a particular protein complex. A “purified protein complex” may be obtained from natural or recombinant host cells or other body samples by standard purification techniques, or by chemical synthesis.

The term “modified protein complex” refers to a protein complex present in a composition that is different from that found in nature, in its native or original cellular or body environment. The term “modification” as used herein refers to all modifications of a protein or protein complex encompassed by the present invention including cleavage and addition or removal of a group. In some embodiments, the “modified protein complex” comprises at least one subunit that is modified, i.e., different from that found in nature, in its native or original cellular or body environment. The “modified subunit” may be, e.g., a derivative or fragment of the native subunit from which it derives from.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus, internally, or at the carboxyl-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. They can be, for example, at least and/or including 10, 15, 20, 25, 30, 35, 40, 45, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340 or more long so long as they are less than the length of the full-length polypeptide. Alternatively, they can be no longer than and/or excluding such a range so long as they are less than the length of the full-length polypeptide.

The term “tag” as used herein is meant to be understood in its broadest sense and to include, but is not limited to any suitable enzymatic, fluorescent, or radioactive labels and suitable epitopes, including but not limited to HA-tag, Myc-tag, T7, His-tag, FLAG-tag, Calmodulin binding proteins, glutathione-S-transferase, strep-tag, KT3-epitope, EEF-epitopes, green-fluorescent protein and variants thereof.

The term “nucleosome” refers to the fundamental unit of chromatin. The term “chromatin” refer to the larger-scale nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a “nucleosome” core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

The term “histone” refers to highly alkaline proteins found in eukaryotic cell nuclei that package and order DNA into structural units called nucleosomes. They are the chief protein components of chromatin, acting as spools around which DNA winds, and play a role in gene regulation. In certain embodiments, the histone is histone H2A (e.g., human, mouse, rat, and/or Xenopus, optionally canonical Histone H2A). In certain embodiments, the histone is histone H2B (e.g., human, mouse, rat, and/or Xenopus, optionally canonical Histone H2B). As described below, H2A and H2B sequences, variation, and structure-function relationships are well-known in the art and are functionally similar, such that, for example, working examples described herein use Xenopus H2A and H2B sequences because they are structurally and functionally similar to Human H2A and H2B sequences.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases The accessibility of chromatin is mediated in part by interactions with SWI/SNF (BAF) complexes via interactions with the nucleosome “acidic patch.” The “acidic patch” of a nucleosome is formed from six H2A and two H2B residues, which together create a highly contoured and negatively charged binding interface on the nucleosome surface. This canonical structural region of nucleosomes is well-known in the art (see, for example, Dann et al. (2017) Nature 548:607-611 and Luger et al. (1997) J. Mol. Biol. 272:301-311) and is described further herein. In certain assays useful according to the present invention, nucleosomal interactions with DNA and/or proteins (e.g., SS18-SSX-containing BAF complexes), can be analyzed. Certain such assays measure changes to DNA lengths. The preferential protection against degradation may be due to the DNA being wrapped around one or more histone proteins, preferably an octomer of histone proteins. The threshold size may be the size of a complete turn of the DNA about a histone core+/−22 bases. The threshold size may be between 100 and 160 bases, preferably between 110 and b 140 bases, more preferably between 120 and 130 bases and ideally 125 bases+/−1 base. The threshold size may be a size equal to or greater than 100 bases, more preferably equal to or greater than 110 bases still more preferably equal to or greater than 120 bases and ideally 125 bases or more.

Eukaryotes have chromatin arranged around proteins in the form of nucleosomes, which are the smallest subunits of chromatin and include approximately 146-147 base pairs of DNA wrapped around an octamer of core histone proteins (two each of H2A, H2B, H3, and H4). As used herein, the term “Histone H3” refers to the H3 member of the Histone family, which comprises proteins used to form the structure of nucleosomes in eukaryotic cells. Mammalian cells have three known sequence variants of Histone H3 proteins, denoted H3.1, H3.2 and H3.3, that are highly conserved differing in sequence by only a few amino acids.

As used herein, the term “Histone H3” can refer to H3.1, H3.2, or H3.3 individually or collectively. These amino acid sequences include a methionine as residue number 1 that is cleaved off when the protein is processed. Thus, for example, serine 11 in the Histone H3 amino acid sequences shown in Table 1 below corresponds to serine (Ser) 10 of the present invention. These three protein variants are encoded by at least fifteen different genes/transcripts. Sequences encoding the Histone H3.1 variant are publicly available as HIST1H3A (NM 003529.2; NP_003520.1), HIST1H3B (NM_003537.3; NP 003528.1), HIST1H3C (NM 003531.2; NP_003522.1), HIST1H3D (NM_003530.3; NP 003521.2), HIST1H3E (NM 003532.2; NP_003523.1), HIST1H3F (NM_021018.2; NP 066298.1), HIST1H3G (NM 003534.2; NP_003525.1), HIST1H3H (NM_003536.2; NP_003527.1), HIST1H3I (NM 003533.2; NP_003524.1), and HIST1H3J (NM_003535.2; NP_003526.1). Sequences encoding the Histone H3.2 variant are publicly available as HIST2H3A (NM_001005464.2; NP 001005464.1), HIST2H3C (NM_021059.2; NP_066403.2), and HIST2H3D (NM_001123375.1; NP 001116847.1). Sequences encoding the Histone H3.3 variant are publicly available as H3F3A (NM_002107.3; NP_002098.1) and H3F3B (NM 005324.3; NP_005315.1). See U.S. Pat. Publ. 2012/0202843 for additional details. Moreover, polypeptide sequences for Histone H3 orthologs, as well as nucleic acid sequences that encode such polypeptides, are well-known in many species, and include, for example, Histone H3.1 orthologs in mice (NM_013550.4; NP_038578.2), chimpanzee (XM_527253.4; XP_527253.2), monkey (XM_001088298.2; XP 001088298.1), dog (XM_003434195.1; XP 003434243.1), cow (XM_002697460.1; XP_002697506.1), rat (XM_001055231.2; XP 001055231.1), and zebrafish (NM_001100173.1; NP_001093643.1). Histone H3.2 orthologs in mice (NM_178215.1; NP_835587.1), chimpanzee (XM_524859.4; XP_524859.2), monkey (XM_001084245.2; XP_001084245.1), dog (XM_003640147.1; XP 003640195.1), cow (XM_002685500.1; XP_002685546.1), rat (NM_001107698.1; NP 001101168.1), chicken (XM_001233027.2; XP_001233028.1), and zebrafish (XM_002662732.1; XP_002662778.1). Similarly, Histone H3.3 orthologs in mice (XM_892026.4; XP_897119.3), monkey (XM_001085836.2; XP_001085836.1), cow (NM_001099370.1; NP 001092840.1), rat (NM_053985.2; NP_446437.1), chicken (NM_205296.1; NP_990627.1), and zebrafish (NM_200003.1; NP_956297.1), are well-known. Representative Histone H3 orthologs are provided in Table 1.

As used herein, the term “Histone H2” can refer to H2A or H2B individually or collectively. The structure of H2A consists of histone fold domain extended by a short alphaC-helix and has both N- and C-terminal tails. The alphaC-helix and C-terminal tail form a docking domain that locks the H2A-H2B dimer onto the surface of H3-H4 tetramer. H2A protein sequences, and nucleic acids encoding same, are well-known in the art and include many useful variants, including canonical H2A, H2A.1, H2A.B, H2A.L, H2A.P, H2A.W, H2A.X, H2A.Z, and macroH2A (see Draizen et al. (2016) Database PMID: 26989147 and HistoneDB 2.0 available on the World Wide Web). The structure of H2B consists of histone fold with a long flexible N-terminal tail which protrudes between the DNA gyres. H2B interats with H4 in the nucleosome vore via four helix bundle motif and alphaC-helix of H2B decorates the nucleosome surface. H2B protein sequences, and nucleic acids ecndogin same, are well-known in the art and include many useful variants, including canonical H2B, H2B.1, H2B.W, H2B.Z, sperm H2B, and subH2B (see Draizen et al. (2016) Database PMID: 26989147 and HistoneDB 2.0 available on the World Wide Web).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention are well-known in the art and readily available on publicly available databases, such as the National Center for Biotechnology Information (NCBI). In addition, nucleic acid and amino acid sequence information for the SS18, SSX, SS18-SSX fusion proteins of the present invention are provided below.

TABLE 1
SEQ ID NO: 1 Human SSX1 cDNA Sequence variant 1 (NM_001278691.2; CDS:
148-714)

| | |
|---|---|
| 1 | accactgctg ccgacctcgc aaccactgct ttgtctctga atagagacag ggtttcctta |
| 61 | tgttggccga actgggcttg acctcctcgg ctcaagtgat cctcccacct cggcctcgga |
| 121 | actacaggtg agactgctcc tggtgccatg aacggagacg acacctttgc aaagagaccc |
| 181 | agggatgatg ctaaagcatc agagaagaga agcaaggcct ttgatgatat tgccacatac |
| 241 | ttctctaaga aagagtggaa aaagatgaaa tactcggaga aaatcagcta tgtgtatatg |
| 301 | aagagaaact ataaggccat gactaaacta ggtttcaaag tcaccctccc acctttcatg |
| 361 | tgtaataaac aggccacaga cttccagggg aatgattttg ataatgacca taaccgcagg |
| 421 | attcaggttg aacatcctca gatgactttc ggcaggctcc acagaatcat cccgaagatc |
| 481 | atgcccaaga agccagcaga ggacgaaaat gattcgaagg gagtgtcaga agcatctggc |
| 541 | ccacaaaacg atgggaaaca actgcacccc ccaggaaaag caaatatttc tgagaagatt |
| 601 | aataagagat ctggacccaa aagggggaaa catgcctgga cccacagact gcgtgagaga |
| 661 | aagcagctgg tgatttatga agagatcagt gaccctgagg aagatgacga gtaactcccc |
| 721 | tgggggatac gacacatgcc cttgatgaga agcagaacgt ggtgaccttt cacgaacatg |
| 781 | ggcatggctg cggctccctc gtcatcaggt gcatagcaag tgaaagcaag tgttcacaac |
| 841 | ggtgaaactt gagcgtcatt tttcttagtg tgccaagagt tcgatgttag tgtttccatt |
| 901 | gtattttctt acagtgtgcc attctgttag atactatcct tataattgat gagcaagaca |
| 961 | tactgaatgc atatttcggt ttgtgtatcc atgcacctac gtcagaaaac aagtattgtc |
| 1021 | aggtattctc tccatagaac agcactatcc tcatctctcc ccagatgtga ctactgaggg |
| 1081 | cagttctgag tgtttaattt cagacttttt cctctgcatt tacacacaca cacacacaca |
| 1141 | cacgcacaca cacacaccaa gtaccagtat aagcatctcc catctgcttt tcccattgcc |
| 1201 | atgcgtcctg gtcaagcccc cctcactctg tttcctgttc agcatgtact cccctcatcc |
| 1261 | gattcccctg tatcagtcac tgacagttaa taaacctttg caaacgttc |

SEQ ID NO: 2 Human SSX1 cDNA Sequence variant 2 (NM_005635.4; CDS: 62-
628)

| | |
|---|---|
| 1 | accactgctg ccgacctcgc aaccactgct ttgtctctga agtgagactg ctcctggtgc |
| 61 | catgaacgga gacgacacct ttgcaaagag acccagggat gatgctaaag catcagagaa |
| 121 | gagaagcaag gcctttgatg atattgccac atacttctct aagaaagagt ggaaaaagat |
| 181 | gaaatactcg gagaaaatca gctatgtgta tatgaagaga aactataagg ccatgactaa |
| 241 | actaggtttc aaagtcaccc tcccaccttt catgtgtaat aaacaggcca cagacttcca |
| 301 | ggggaatgat tttgataatg accataaccg caggattcag gttgaacatc ctcagatgac |
| 361 | tttcggcagg ctccacagaa tcatcccgaa gatcatgccc aagaagccag cagaggacga |
| 421 | aaatgattcg aagggagtgt cagaagcatc tggcccacaa aacgatggga aacaactgca |
| 481 | ccccccagga aaagcaaata tttctgagaa gattaataag agatctggac ccaaaagggg |
| 541 | gaaacatgcc tggacccaca gactgcgtga gagaaagcag ctggtgattt atgaagagat |
| 601 | cagtgaccct gaggaagatg acgagtaact cccctggggg atacgacaca tgcccttgat |
| 661 | gagaagcaga acgtggtgac ctttcacgaa catgggcatg gctgcggctc cctcgtcatc |
| 721 | aggtgcatag caagtgaaag caagtgttca caacggtgaa acttgagcgt catttttctt |
| 781 | agtgtgccaa gagttcgatg ttagtgtttc cattgtattt tcttacagtg tgccattctg |
| 841 | ttagatacta tccttataat tgatgagcaa gacatactga atgcatattt cggtttgtgt |
| 901 | atccatgcac ctacgtcaga aaacaagtat tgtcaggtat tctctccata gaacagcact |
| 961 | atcctcatct ctccccagat gtgactactg agggcagttc tgagtgttta atttcagact |
| 1021 | ttttcctctg catttacaca cacacacaca cacacacgca cacacacaca ccaagtacca |
| 1081 | gtataagcat ctcccatctg cttttcccat tgccatgcgt cctggtcaag cccccctcac |
| 1141 | tctgtttcct gttcagcatg tactcccctc atccgattcc cctgtatcag tcactgacag |
| 1201 | ttaataaacc tttgcaaacg ttc |

SEQ ID NO: 3 Human SSX1 Amino Acid Sequence isoform 1 (NP_001265620.1
and NP_005626.1)

| | |
|---|---|
| 1 | mngddtfakr prddakasek rskafddiat yfskkewkkm kysekisyvy mkrnykamtk |
| 61 | lgfkvtlppf mcnkqatdfq gndfdndhnr riqvehpqmt fgrlhriipk impkkpaede |
| 121 | ndskgvseas gpqndgkqlh ppgkanisek inkrsgpkrg khawthrlre rkqlviyeei |
| 181 | sdpeedde |

SEQ ID NO: 4 Human SSX2 cDNA Sequence variant 1 (NM_003147.5; CDS: 137-
808)

| | |
|---|---|
| 1 | gggattggct actttaagtt cagagtacgc atgctctgac tttctctctc tttcgattct |
| 61 | tccatactca gagtacgcac ggtctgattt tctctttgga ttcttccaaa atcagagtca |
| 121 | gactgctccc ggtgccatga acggagacga cgcctttgca aggagaccca cggttggtgc |
| 181 | tcaaatacca gagaagatcc aaaaggcctt cgatgatatt gccaaatact tctctaagga |
| 241 | agagtgggaa aagatgaaag cctcggagaa aatcttctat gtgtatatga agagaaagta |
| 301 | tgaggctatg actaaactag gtttcaaggc caccctccca cctttcatgt gtaataaacg |
| 361 | ggccgaagac ttccagggga atgatttgga taatgaccct aaccgtggga atcaggttga |
| 421 | acgtcctcag atgactttcg gcaggctcca gggaatctcc ccgaagatca tgcccaagaa |
| 481 | gccagcagag gaaggaaatg attcggagga agtgccagaa gcatctggcc cacaaaatga |
| 541 | tgggaaagag ctgtgccccc cgggaaaacc aactacctct gagaagattc acgagagatc |
| 601 | tggaaatagg gaggcccaag aaaaggaaga gagacgcgga acagctcatc ggtggagcag |
| 661 | tcagaacaca cacaacattg gtcgattcag tttgtcaact tctatgggtg cagttcatgg |
| 721 | tacccccaaa acaattacac acaacaggga cccaaaaggg gggaacatgc ctggacccac |
| 781 | agactgcgtg agagaaaaca gctggtgatt tatgaagaga tcagcgaccc tgaggaagat |
| 841 | gacgagtaac tcccctcagg gatacgacac atgcccatga tgagaagcag aacgtggtga |
| 901 | cctttcacga acatgggcat ggctgcggac ccctcgtcat caggtgcata gcaagtgaaa |
| 961 | gcaagtgttc acaacagtga aaagttgagc gtcatttttc ttagtgtgcc aagagttcga |
| 1021 | tgttagcgtt tacgttgtat tttcttacac tgtgtcattc tgttagatac taacattttc |
| 1081 | attgatgagc aagacatact taatgcatat tttggtttgt gtatccatgc acctacctta |
| 1141 | gaaaacaagt attgtcggtt acctctgcat ggaacagcat taccctcctc tctccccaga |
| 1201 | tgtgactact gagggcagtt ctgagtgttt aatttcagat tttttcctct gcatttacac |
| 1261 | acacacgcac acaaaccaca ccacacacac acacacacac acacacacac acacacacac |
| 1321 | acaccaagta ccagtataag catctgccat ctgcttttcc cattgccatg cgtcctggtc |
| 1381 | aagctcccct cactctgttt cctggtcagc atgtactccc ctcatccgat tcccctgtag |
| 1441 | cagtcactga cagttaataa acctttgcaa acgttcaaaa aaaaaaaaaa aaaa |

SEQ ID NO: 5 Human SSX2 Amino Acid Sequence isoform 1 (NP_003138.3)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igrfslstsm gavhgtpkti thnrdpkggn mpgptdcvre nsw |

SEQ ID NO: 6 Human SSX2 cDNA Sequence variant 2 (NM_175698.2; CDS: 137-
703)

| | |
|---|---|
| 1 | gggattggct actttaagtt cagagtacgc atgctctgac tttctctctc tttcgattct |
| 61 | tccatactca gagtacgcac ggtctgattt ttctttgga ttcttccaaa atcagagtca |
| 121 | gactgctccc ggtgccatga acggagacga cgcctttgca aggagaccca cggttggtgc |
| 181 | tcaaatacca gagaagatcc aaaaggcctt cgatgatatt gccaaatact tctctaagga |
| 241 | agagtgggaa aagatgaaag cctcggagaa aatcttctat gtgtatatga agagaaagta |
| 301 | tgaggctatg actaaactag gtttcaaggc caccctccca cctttcatgt gtaataaacg |
| 361 | ggccgaagac ttccagggga atgatttgga taatgaccct aaccgtggga atcaggttga |
| 421 | acgtcctcag atgactttcg gcaggctcca gggaatctcc ccgaagatca tgcccaagaa |
| 481 | gccagcagag gaaggaaatg attcggagga agtgccagaa gcatctggcc cacaaaatga |
| 541 | tgggaaagag ctgtgccccc cgggaaaacc aactacctct gagaagattc acgagagatc |
| 601 | tggacccaaa aggggggaac atgcctggac ccacagactg cgtgagagaa aacagctggt |
| 661 | gatttatgaa gagatcagcg accctgagga agatgacgag taactcccct cagggatacg |
| 721 | acacatgccc atgatgagaa gcagaacgtg gtgacctttc acgaacatgg gcatggctgc |
| 781 | ggacccctcg tcatcaggtg catagcaagt gaaagcaagt gttcacaaca gtgaaaagtt |
| 841 | gagcgtcatt tttcttagtg tgccaagagt tcgatgttag cgtttacgtt gtattttctt |
| 901 | acactgtgtc attctgttag atactaacat tttcattgat gagcaagaca tacttaatgc |
| 961 | atattttggt ttgtgtatcc atgcacctac cttagaaaac aagtattgtc ggttacctct |
| 1021 | gcatggaaca gcattaccct cctctctccc cagatgtgac tactgagggc agttctgagt |
| 1081 | gtttaatttc agattttttc ctctgcattt acacacacac gcacacaaac cacaccacac |
| 1141 | acacacacac acacacacac acacacacac acacacacca agtaccagta taagcatctg |
| 1201 | ccatctgctt ttcccattgc catgcgtcct ggtcaagctc ccctcactct gtttcctggt |
| 1261 | cagcatgtac tcccctcatc cgattcccct gtagcagtca ctgacagtta ataaaccttt |
| 1321 | gcaaacgttc aaaaaaaaaa aaaaaaaa |

SEQ ID NO: 7 Human SSX2 Amino Acid Sequence isoform 2 (NP_783629.1)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgpkrg ehawthrlre rkqlviyeei |
| 181 | sdpeedde |

SEQ ID NO: 8 Human SSX2 cDNA Sequence variant 3 (NM_001278697.1; CDS:
137-781)

| | |
|---|---|
| 1 | gggattggct actttaagtt cagagtacgc atgctctgac tttctctctc tttcgattct |
| 61 | tccatactca gagtacgcac ggtctgattt tctctttgga ttcttccaaa atcagagtca |
| 121 | gactgctccc ggtgccatga acggagacga cgcctttgca aggagaccca cggttggtgc |
| 181 | tcaaatacca gagaagatcc aaaaggcctt cgatgatatt gccaaatact tctctaagga |
| 241 | agagtgggaa aagatgaaag cctcggagaa aatcttctat gtgtatatga agagaaagta |
| 301 | tgaggctatg actaaactag gtttcaaggc caccctccca cctttcatgt gtaataaacg |
| 361 | ggccgaagac ttccagggga atgatttgga taatgaccct aaccgtggga atcaggttga |
| 421 | acgtcctcag atgactttcg gcaggctcca gggaatctcc ccgaagatca tgcccaagaa |
| 481 | gccagcagag gaaggaaatg attcggagga agtgccagaa gcatctggcc cacaaaatga |
| 541 | tgggaaagag ctgtgccccc cgggaaaacc aactacctct gagaagattc acgagagatc |
| 601 | tggaaatagg gaggcccaag aaaaggaaga gagacgcgga acagctcatc ggtggagcag |
| 661 | tcagaacaca cacaacattg gacccaaaag gggggaacat gcctggaccc acagactgcg |
| 721 | tgagagaaaa cagctggtga tttatgaaga gatcagcgac cctgaggaag atgacgagta |
| 781 | actcccctca gggatacgac acatgcccat gatgagaagc agaacgtggt gacctttcac |
| 841 | gaacatgggc atggctgcgg acccctcgtc atcaggtgca tagcaagtga aagcaagtgt |
| 901 | tcacaacagt gaaaagttga gcgtcatttt tcttagtgtg ccaagagttc gatgttagcg |
| 961 | tttacgttgt attttcttac actgtgtcat tctgttagat actaacattt tcattgatga |
| 1021 | gcaagacata cttaatgcat attttggttt gtgtatccat gcacctacct tagaaaacaa |
| 1081 | gtattgtcgg ttacctctgc atggaacagc attaccctcc tctctcccca gatgtgacta |
| 1141 | ctgagggcag ttctgagtgt ttaatttcag attttttcct ctgcatttac acacacacgc |
| 1201 | acacaaacca caccacacac acacacacac acacacacac acacacacac acacaccaag |
| 1261 | taccagtata agcatctgcc atctgctttt cccattgcca tgcgtcctgg tcaagctccc |
| 1321 | ctcactctgt ttcctggtca gcatgtactc ccctcatccg attcccctgt agcagtcact |
| 1381 | gacagttaat aaacctttgc aaacgttcaa aaaaaaaaaa aaaaaa |

SEQ ID NO: 9 Human SSX2 Amino Acid Sequence isoform 3 (NP_001265626.1)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igpkrgehaw thrlrerkql viyeeisdpe edde |

SEQ ID NO: 10 Human SSX2B cDNA Sequence variant 1 (NM_001278701.2; CDS:
88-759)

| | |
|---|---|
| 1 | ctttcgattc ttccatactc agagtacgca cggtctgatt ttctctttgg attcttccaa |
| 61 | aatcagagtc agactgctcc cggtgccatg aacggagacg acgcctttgc aaggagaccc |
| 121 | acggttggtg ctcaaatacc agagaagatc caaaaggcct tcgatgatat tgccaaatac |
| 181 | ttctctaagg aagagtggga aaagatgaaa gcctcggaga aaatcttcta tgtgtatatg |
| 241 | aagagaaagt atgaggctat gactaaacta ggtttcaagg ccaccctccc acctttcatg |
| 301 | tgtaataaac gggccgaaga cttccagggg aatgatttgg ataatgaccc taaccgtggg |
| 361 | aatcaggttg aacgtcctca gatgactttc ggcaggctcc agggaatctc cccgaagatc |
| 421 | atgcccaaga agccagcaga ggaaggaaat gattcggagg aagtgccaga agcatctggc |
| 481 | ccacaaaatg atgggaaaga gctgtgcccc ccgggaaaac caactacctc tgagaagatt |
| 541 | cacgagagat ctggaaatag ggaggcccaa gaaaaggaag agagacgcgg aacagctcat |
| 601 | cggtggagca gtcagaacac acacaacatt ggtcgattca gtttgtcaac ttctatgggt |
| 661 | gcagttcatg gtacccccaa aacaattaca cacaacaggg acccaaaagg ggggaacatg |
| 721 | cctggaccca cagactgcgt gagagaaaac agctggtgat ttatgaagag atcagcgacc |
| 781 | ctgaggaaga tgacgagtaa ctcccctcag ggatacgaca catgcccatg atgagaagca |
| 841 | gaacgtggtg acctttcacg aacatgggca tggctgcgga cccctcgtca tcaggtgcat |
| 901 | agcaagtgaa agcaagtgtt cacaacagtg aaaagttgag cgtcattttt cttagtgtgc |
| 961 | caagagttcg atgttagcgt ttacgttgta ttttcttaca ctgtgtcatt ctgttagata |
| 1021 | ctaacatttt cattgatgag caagacatac ttaatgcata ttttggtttg tgtatccatg |
| 1081 | cacctacctt agaaaacaag tattgtcggt tacctctgca tggaacagca ttaccctect |
| 1141 | ctctccccag atgtgactac tgagggcagt tctgagtgtt taatttcaga ttttttcctc |
| 1201 | tgcatttaca cacacacgca cacaaaccac accacacaca cacacacaca cacacacaca |
| 1261 | cacacacaca cacaccaagt accagtataa gcatctgcca tctgcttttc ccattgccat |
| 1321 | gcgtcctggt caagctcccc tcactctgtt tcctggtcag catgtactcc cctcatccga |
| 1381 | ttcccctgta gcagtcactg acagttaata aacctttgca aacgttc |

SEQ ID NO: 11 Human SSX2B Amino Acid Sequence isoform 1 (NP_001265630.1)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igrfslstsm gavhgtpkti thnrdpkggn mpgptdcvre nsw |

SEQ ID NO: 12 Human SSX2B cDNA Sequence variant 2 (NM_001164417.3; CDS:
88-654)

| | |
|---|---|
| 1 | ctttcgattc ttccatactc agagtacgca cggtctgatt ttctctttgg attcttccaa |
| 61 | aatcagagtc agactgctcc cggtgccatg aacggagacg acgcctttgc aaggagaccc |
| 121 | acggttggtg ctcaaatacc agagaagatc caaaaggcct tcgatgatat tgccaaatac |
| 181 | ttctctaagg aagagtggga aaagatgaaa gcctcggaga aaatcttcta tgtgtatatg |
| 241 | aagagaaagt atgaggctat gactaaacta ggtttcaagg ccaccctccc acctttcatg |
| 301 | tgtaataaac gggccgaaga cttccagggg aatgatttgg ataatgaccc taaccgtggg |
| 361 | aatcaggttg aacgtcctca gatgactttc ggcaggctcc agggaatctc cccgaagatc |
| 421 | atgcccaaga agccagcaga ggaaggaaat gattcggagg aagtgccaga agcatctggc |
| 481 | ccacaaaatg atgggaaaga gctgtgcccc ccgggaaaac caactacctc tgagaagatt |
| 541 | cacgagagat ctggacccaa aaggggggaa catgcctgga cccacagact gcgtgagaga |
| 601 | aaacagctgg tgatttatga agagatcagc gaccctgagg aagatgacga gtaactcccc |
| 661 | tcagggatac gacacatgcc catgatgaga agcagaacgt ggtgaccttt cacgaacatg |
| 721 | ggcatggctg cggacccctc gtcatcaggt gcatagcaag tgaaagcaag tgttcacaac |
| 781 | agtgaaaagt tgagcgtcat ttttcttagt gtgccaagag ttcgatgtta gcgtttacgt |
| 841 | tgtattttct tacactgtgt cattctgtta gatactaaca ttttcattga tgagcaagac |
| 901 | atacttaatg catattttgg tttgtgtatc catgcaccta ccttagaaaa caagtattgt |
| 961 | cggttacctc tgcatggaac agcattaccc tectctctcc ccagatgtga ctactgaggg |
| 1021 | cagttctgag tgtttaattt cagatttttt cctctgcatt tacacacaca cgcacacaaa |
| 1081 | ccacaccaca cacacacaca cacacacaca cacacacaca cacacacacc aagtaccagt |
| 1141 | ataagcatct gccatctgct tttcccattg ccatgcgtcc tggtcaagct cccctcactc |
| 1201 | tgtttcctgg tcagcatgta ctcccctcat ccgattcccc tgtagcagtc actgacagtt |
| 1261 | aataaacctt tgcaaacgtt c |

SEQ ID NO: 13 Human SSX2B Amino Acid Sequence isoform 2 (NP_001157889.1)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgpkrg ehawthrlre rkqlviyeei |
| 181 | sdpeedde |

SEQ ID NO: 14 Human SSX2B cDNA Sequence variant 3 (NM_001278702.2; CDS:
88-732)

| | |
|---|---|
| 1 | ctttcgattc ttccatactc agagtacgca cggtctgatt ttctctttgg attcttccaa |
| 61 | aatcagagtc agactgctcc cggtgccatg aacggagacg acgcctttgc aaggagaccc |
| 121 | acggttggtg ctcaaatacc agagaagatc caaaaggcct tcgatgatat tgccaaatac |
| 181 | ttctctaagg aagagtggga aaagatgaaa gcctcggaga aaatcttcta tgtgtatatg |
| 241 | aagagaaagt atgaggctat gactaaacta ggtttcaagg ccaccctccc acctttcatg |
| 301 | tgtaataaac gggccgaaga cttccagggg aatgatttgg ataatgaccc taaccgtggg |
| 361 | aatcaggttg aacgtcctca gatgactttc ggcaggctcc agggaatctc cccgaagatc |
| 421 | atgcccaaga agccagcaga ggaaggaaat gattcggagg aagtgccaga agcatctggc |
| 481 | ccacaaaatg atgggaaaga gctgtgcccc ccgggaaaac caactacctc tgagaagatt |
| 541 | cacgagagat ctggaaatag ggaggcccaa gaaaaggaag agagacgcgg aacagctcat |
| 601 | cggtggagca gtcagaacac acacaacatt ggacccaaaa ggggggaaca tgcctggacc |
| 661 | cacagactgc gtgagagaaa acagctggtg atttatgaag agatcagcga ccctgaggaa |
| 721 | gatgacgagt aactcccctc agggatacga cacatgccca tgatgagaag cagaacgtgg |
| 781 | tgacctttca cgaacatggg catggctgcg gacccctcgt catcaggtgc atagcaagtg |
| 841 | aaagcaagtg ttcacaacag tgaaaagttg agcgtcattt ttcttagtgt gccaagagtt |
| 901 | cgatgttagc gtttacgttg tattttctta cactgtgtca ttctgttaga tactaacatt |
| 961 | ttcattgatg agcaagacat acttaatgca tattttggtt tgtgtatcca tgcacctacc |
| 1021 | ttagaaaaca agtattgtcg gttacctctg catggaacag cattaccctc ctctctcccc |
| 1081 | agatgtgact actgagggca gttctgagtg tttaatttca gattttttcc tctgcattta |
| 1141 | cacacacacg cacacaaacc acaccacaca cacacacaca cacacacaca cacacacaca |
| 1201 | cacacaccaa gtaccagtat aagcatctgc catctgcttt tcccattgcc atgcgtcctg |
| 1261 | gtcaagctcc cctcactctg tttcctggtc agcatgtact cccctcatcc gattcccctg |
| 1321 | tagcagtcac tgacagttaa taaacctttg caaacgttc |

SEQ ID NO: 15 Human SSX2B Amino Acid Sequence isoform 3 (NP_001265631.1)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igpkrgehaw thrlrerkql viyeeisdpe edde |

SEQ ID NO: 16 Human SSX4 cDNA Sequence variant 1 (NM_005636.4; CDS: 47-
613)

| | |
|---|---|
| 1 | gcccttttga ttcttccaca atcagggtga gactgctccc agtgccatga acggagacga |
| 61 | cgcctttgca aggagaccca gggatgatgc tcaaatatca gagaagttac gaaaggcctt |
| 121 | cgatgatatt gccaaatact tctctaagaa agagtgggaa aagatgaaat cctcggagaa |
| 181 | aatcgtctat gtgtatatga agctaaacta tgaggtcatg actaaactag gtttcaaggt |
| 241 | caccctccca cctttcatgc gtagtaaacg ggctgcagac ttccacggga atgattttgg |
| 301 | taacgatcga aaccacagga atcaggttga acgtcctcag atgactttcg gcagcctcca |
| 361 | gagaatcttc ccgaagatca tgcccaagaa gccagcagag gaagaaaatg gtttgaagga |
| 421 | agtgccagag gcatctggcc cacaaaatga tgggaaacag ctgtgccccc cgggaaatcc |
| 481 | aagtaccttg gagaagatta acaagacatc tggacccaaa agggggaaac atgcctggac |
| 541 | ccacagactg cgtgagagaa agcagctggt ggtttatgaa gagatcagcg accctgagga |
| 601 | agatgacgag taactcccct cggggatatg acacatgccc atgatgagaa gcagaacgtg |
| 661 | gtgacctttc acgaacatgg gcatggctgc ggacccctcg tcatcaggtg catagcaagt |
| 721 | gaaagcaagt gttcacaaca gtgaaaagtt gagcgtcatt tttcttagtg tgccaagagt |
| 781 | tcgatgttgg cgtttccgct gtattttctt gcagtgtgcc attctgttag acattagcgt |
| 841 | tttcgttgat gagcaagaca tgcttaatgc atatttcggc ttgtgtatcc atgcacctac |
| 901 | ctcagaaaac aagtattgtc aggtattctc tccatagaac agcactaccc tectctctcc |
| 961 | ccagatgtga ctactgaggg gaggtctgag tgtttaattt ccgatttttt cctctgcatt |
| 1021 | tacacacaca ccacacacgc acacacacac accaagtacc agtataagca tctcccatct |
| 1081 | gcttttctcc attgccatgc gacctggtca agcccccctc actctgtttc ctgttcagca |
| 1141 | tgtactcccc tcatccgatt ccgttgtatc agtcactgac agttaataaa cctttgcaaa |
| 1201 | cgttcccca |

SEQ ID NO: 17 Human SSX4 Amino Acid Sequence isoform 1 (NP_005627.1)

| | |
|---|---|
| 1 | mngddafarr prddaqisek lrkafddiak yfskkewekm kssekivyvy mklnyevmtk |
| 61 | lgfkvtlppf mrskraadfh gndfgndrnh rnqverpqmt fgslqrifpk impkkpaeee |
| 121 | nglkevpeas gpqndgkqlc ppgnpstlek inktsgpkrg khawthrlre rkqlvvyeei |
| 181 | sdpeedde |

SEQ ID NO: 18 Human SSX4 cDNA Sequence variant 2 (NM_175729.1; CDS: 59-

| | |
|---|---|
| 520) | |
| 1 | acacgccgat ttgccctttt gattcttcca caatcagggt gagactgctc ccagtgccat |
| 61 | gaacggagac gacgcctttg caaggagacc cagggatgat gctcaaatat cagagaagtt |
| 121 | acgaaaggcc ttcgatgata ttgccaaata cttctctaag aaagagtggg aaaagatgaa |
| 181 | atcctcggag aaaatcgtct atgtgtatat gaagctaaac tatgaggtca tgactaaact |
| 241 | aggtttcaag gtcaccctcc cacctttcat gcgtagtaaa cgggctgcag acttccacgg |
| 301 | gaatgatttt ggtaacgatc gaaaccacag gaatcaggtt gaacgtcctc agatgacttt |
| 361 | cggcagcctc cagagaatct tcccgaagga cccaaaaggg ggaaacatgc ctggacccac |
| 421 | agactgcgtg agagaaagca gctggtggtt tatgaagaga tcagcgaccc tgaggaagat |
| 481 | gacgagtaac tcccctcggg gatatgacac atgcccatga tgagaagcag aacgtggtga |
| 541 | cctttcacga acatgggcat ggctgcggac ccctcgtcat caggtgcata gcaagtgaaa |
| 601 | gcaagtgttc acaacagtga aaagttgagc gtcatttttc ttagtgtgcc aagagttcga |
| 661 | tgttggcgtt tccgctgtat tttcttgcag tgtgccattc tgttagacat tagcgttttc |
| 721 | gttgatgagc aagacatgct taatgcatat ttcggcttgt gtatccatgc acctacctca |
| 781 | gaaaacaagt attgtcaggt attctctcca tagaacagca ctaccctect ctctccccag |
| 841 | atgtgactac tgaggggagg tctgagtgtt taatttccga ttttttcctc tgcatttaca |
| 901 | cacacaccac acacgcacac acacacacca agtaccagta taagcatctc ccatctgctt |
| 961 | ttctccattg ccatgcgacc tggtcaagcc cccctcactc tgtttcctgt tcagcatgta |
| 1021 | ctcccctcat ccgattccgt tgtatcagtc actgacagtt aataaacctt tgcaaacgtt |
| 1081 | caaaaaaaaa aaaaaaaaaa aaaaaaaaaa aaaa |

SEQ ID NO: 19 Human SSX4 Amino Acid Sequence isoform 2 (NP_783856.1)

| | |
|---|---|
| 1 | mngddafarr prddaqisek lrkafddiak yfskkewekm kssekivyvy mklnyevmtk |
| 61 | lgfkvtlppf mrskraadfh gndfgndrnh rnqverpqmt fgslqrifpk dpkggnmpgp |
| 121 | tdcvressww fmkrsatlrk mtsnsprgyd tcp |

SEQ ID NO: 20 Human SSX4B cDNA Sequence variant 1 (NM_001034832.3; CDS:
70-636)

| | |
|---|---|
| 1 | tcagagtacg cacacgccga tttgcccttt tgattcttcc acaatcaggg tgagactgct |
| 61 | cccagtgcca tgaacggaga cgacgccttt gcaaggagac ccagggatga tgctcaaata |
| 121 | tcagagaagt tacgaaaggc cttcgatgat attgccaaat acttctctaa gaaagagtgg |
| 181 | gaaaagatga aatcctcgga gaaaatcgtc tatgtgtata tgaagctaaa ctatgaggtc |
| 241 | atgactaaac taggtttcaa ggtcaccctc ccacctttca tgcgtagtaa acgggctgca |
| 301 | gacttccacg ggaatgattt tggtaacgat cgaaaccaca ggaatcaggt tgaacgtcct |
| 361 | cagatgactt tcggcagcct ccagagaatc ttcccgaaga tcatgcccaa gaagccagca |
| 421 | gaggaagaaa atggtttgaa ggaagtgcca gaggcatctg gcccacaaaa tgatgggaaa |
| 481 | cagctgtgcc ccccgggaaa tccaagtacc ttggagaaga ttaacaagac atctggaccc |
| 541 | aaaaggggga aacatgcctg gacccacaga ctgcgtgaga gaaagcagct ggtggtttat |
| 601 | gaagagatca gcgaccctga ggaagatgac gagtaactcc cctcggggat atgacacatg |
| 661 | cccatgatga gaagcagaac gtggtgacct ttcacgaaca tgggcatggc tgcggacccc |
| 721 | tcgtcatcag gtgcatagca agtgaaagca agtgttcaca acagtgaaaa gttgagcgtc |
| 781 | atttttctta gtgtgccaag agttcgatgt tggcgtttcc gctgtatttt cttgcagtgt |
| 841 | gccattctgt tagacattag cgttttogtt gatgagcaag acatgcttaa tgcatatttc |
| 901 | ggcttgtgta tccatgcacc tacctcagaa aacaagtatt gtcaggtatt ctctccatag |
| 961 | aacagcacta ccctcctctc tccccagatg tgactactga ggggaggtct gagtgtttaa |
| 1021 | tttccgattt tttcctctgc atttacacac acaccacaca cgcacacaca cacaccaagt |
| 1081 | accagtataa gcatctccca tctgcttttc tccattgcca tgcgacctgg tcaagccccc |
| 1141 | ctcactctgt ttcctgttca gcatgtactc ccctcatccg attccgttgt atcagtcact |
| 1201 | gacagttaat aaacctttgc aaacgttcaa aaaaaaaaaa aaaa |

SEQ ID NO: 21 Human SSX4B Amino Acid Sequence isoform 1 (NP_001030004.1)

| | |
|---|---|
| 1 | mngddafarr prddaqisek lrkafddiak yfskkewekm kssekivyvy mklnyevmtk |
| 61 | lgfkvtlppf mrskraadfh gndfgndrnh rnqverpqmt fgslqrifpk impkkpaeee |
| 121 | nglkevpeas gpqndgkqlc ppgnpstlek inktsgpkrg khawthrlre rkqlvvyeei |
| 181 | sdpeedde |

SEQ ID NO: 22 Human SSX4B cDNA Sequence variant 2 (NM_001040612.2; CDS:
70-531)

| | |
|---|---|
| 1 | tcagagtacg cacacgccga tttgcccttt tgattcttcc acaatcaggg tgagactgct |
| 61 | cccagtgcca tgaacggaga cgacgccttt gcaaggagac ccagggatga tgctcaaata |
| 121 | tcagagaagt tacgaaaggc cttcgatgat attgccaaat acttctctaa gaaagagtgg |
| 181 | gaaaagatga aatcctcgga gaaaatcgtc tatgtgtata tgaagctaaa ctatgaggtc |
| 241 | atgactaaac taggtttcaa ggtcaccctc ccacctttca tgcgtagtaa acgggctgca |
| 301 | gacttccacg ggaatgattt tggtaacgat cgaaaccaca ggaatcaggt tgaacgtcct |
| 361 | cagatgactt tcggcagcct ccagagaatc ttcccgaagg acccaaaagg gggaaacatg |
| 421 | cctggaccca cagactgcgt gagagaaagc agctggtggt ttatgaagag atcagcgacc |
| 481 | ctgaggaaga tgacgagtaa ctcccctcgg ggatatgaca catgcccatg atgagaagca |
| 541 | gaacgtggtg acctttcacg aacatgggca tggctgcgga cccctcgtca tcaggtgcat |
| 601 | agcaagtgaa agcaagtgtt cacaacagtg aaaagttgag cgtcattttt cttagtgtgc |
| 661 | caagagttcg atgttggcgt ttccgctgta ttttcttgca gtgtgccatt ctgttagaca |
| 721 | ttagcgtttt cgttgatgag caagacatgc ttaatgcata tttcggcttg tgtatccatg |
| 781 | cacctacctc agaaaacaag tattgtcagg tattctctcc atagaacage actaccctcc |
| 841 | tctctcccca gatgtgacta ctgaggggag gtctgagtgt ttaatttccg attttttcct |
| 901 | ctgcatttac acacacacca cacacgcaca cacacacacc aagtaccagt ataagcatct |
| 961 | cccatctgct tttctccatt gccatgcgac ctggtcaagc ccccctcact ctgtttcctg |
| 1021 | ttcagcatgt actcccctca tccgattccg ttgtatcagt cactgacagt taataaacct |
| 1081 | ttgcaaacgt tcaaaaaaaa aaaaaaaa |

SEQ ID NO: 23 Human SSX4B Amino Acid Sequence isoform 2 (NP_001035702.1)

| | |
|---|---|
| 1 | mngddafarr prddaqisek lrkafddiak yfskkewekm kssekivyvy mklnyevmtk |
| 61 | lgfkvtlppf mrskraadfh gndfgndrnh rnqverpqmt fgslqrifpk dpkggnmpgp |
| 121 | tdcvressww fmkrsatlrk mtsnsprgyd tcp |

SEQ ID NO: 24 Human SSX3 cDNA Sequence (NM_021014.4; CDS: 91-657)

| | |
|---|---|
| 1 | ctctttcgat tcttccatac tcaagagtac gcacggtctg attttctctt tggattcttc |
| 61 | caaaatcaga gtcagactac tccctgtgcc atgaacggag atgacacctt tgcaaggaga |
| 121 | cccacggttg gtgctcaaat accagagaag atacaaaagg ccttcgatga tattgccaaa |
| 181 | tacttctcta aggaagagtg ggaaaagatg aaagtctcgg agaaaatcgt ctatgtgtat |
| 241 | atgaagagaa agtatgaggc catgactaaa ctaggtttca aggccatcct cccatctttc |
| 301 | atgcgtaata aacgggtcac agacttccag gggaatgatt ttgataatga ccctaaccgt |
| 361 | gggaatcagg ttcaacgtcc tcagatgact ttcggcaggc tccagggaat cttcccgaag |
| 421 | atcatgccca agaagccagc agaggaagga aatgtttcga aggaagtgcc agaagcatct |
| 481 | ggcccacaaa acgatgggaa acagctgtgc cccccgggaa aaccaactac ctctgagaag |
| 541 | attaacatga tatctggacc caaaaggggg gaacatgcct ggacccacag actgcgtgag |
| 601 | agaaagcagc tggtgattta tgaagagatc agcgatcctg aggaagatga tgagtaactc |
| 661 | cccttgggga tatgacacat gcccatgatg agaagcagaa cgtggtgacc tttcacgaac |
| 721 | atgggcatgg ctgtggaccc ctcgtcatca ggtgcatagc aagtgaaagc aagtgttcac |
| 781 | aacagtgaaa agttgagcgt catttttctt agtgtgccaa gagtacgata ttagcgtttc |
| 841 | cattgtattt tcttgaagtg tgtcattctg ttagatatta acattttcac tgatgagcaa |
| 901 | gacatactta atgcatattt tggtttgtgt atccatgcac ctaccttaga aaacaagtat |
| 961 | tgtcagttac ctctgcatgg aacagcatta ccctcctctc tccctagatg tgactactga |
| 1021 | gggcagttct gagtgtttaa tttcagattt tttcctctgc atttacacac acacacaaac |
| 1081 | cacaccacac acacacacac acacacacag acacacacca agtaccagta taagcatctc |
| 1141 | ccatctgctt ttcccattgc catgcgtcct ggtcaggctt ccctcactct gtttcctggt |
| 1201 | cagcatgtac tcccctcatc cgattcccct gtagcagtca ctgacagtaa ataaaccttt |
| 1261 | gcaaacgttc |

SEQ ID NO: 25 Human SSX3 Amino Acid Sequence (NP_066294.1)

| | |
|---|---|
| 1 | mngddtfarr ptvgaqipek iqkafddiak yfskeewekm kvsekivyvy mkrkyeamtk |
| 61 | lgfkailpsf mrnkrvtdfq gndfdndpnr gnqvqrpqmt fgrlqgifpk impkkpaeeg |
| 121 | nvskevpeas gpqndgkqlc ppgkpttsek inmisgpkrg ehawthrlre rkqlviyeei |
| 181 | sdpeedde |

SEQ ID NO: 26 Human SSX5 cDNA Sequence variant 1 (NM_021015.4; CDS:
86 . . . 775)

| | |
|---|---|
| 1 | ctctctctct cgatttttcc acagagtacg cacgctctga ttgtttcgat tcttccaaaa |
| 61 | tcagagacag agtgctcccg gtgccatgaa cggagacgat gcctttgtac ggagacctag |
| 121 | ggttggttct caaataccag agaagatgca aaagcatccc tggagacaag tctgtgaccg |
| 181 | tggaatacat ttggtgaatc tcagtccgtt ctggaaggtg ggaagagagc cagccagcag |
| 241 | cattaaagct ctactgtgtg gcaggggaga agctagggcc ttcgatgata ttgccaaata |
| 301 | cttctctgag aaagagtggg aaaagatgaa agcctcggag aaaatcatct atgtgtatat |
| 361 | gaagagaaag tatgaggcca tgactaaact aggtttcaag gccaccctcc cacctttcat |
| 421 | gcgtaataaa cgggtcgcag acttccaggg gaatgatttt gataatgacc ctaaccgtgg |
| 481 | gaatcaggtt gaacatcctc agatgacttt cggcaggctc cagggaatct tcccgaagat |
| 541 | cacgcccgag aagccagcag aggaaggaaa tgattcgaag ggagtgccag aagcatctgg |
| 601 | cccacagaac aatgggaaac agctgcgccc ctcaggaaaa ctaaatacct ctgagaaggt |
| 661 | taacaagaca tctggaccca aaagggggaa acatgcctgg acccacagag tgcgtgagag |
| 721 | aaagcaactg gtgatttatg aagagatcag cgaccctcag gaagatgacg agtaactccc |
| 781 | ctcggggata tgacacatgc ccatgatgag aagcagaacg tggtgacctt tcacgaacat |
| 841 | gggcatggct gcggatccct cgtcatcagg tgtatagcaa gtgaaagcaa gtgttcacaa |
| 901 | cagtgaaaag ttgagcgtca tttttcttag tgtgccaaga gttcgatgtt agtgtttctg |
| 961 | ttgtattttg ttacagtgtg ccattctgtt agatattagc gttttcactg atgagcaaga |
| 1021 | catacttaat gcatatttca gtttgtgtat ccatgcacct acctcagaaa acaagtatcg |
| 1081 | tcaggtattc tctgcataga acaacactac cctcctctct tcccagatgt gaccactgag |
| 1141 | ggcagttctg agtgtttaat ttcagatttt ttcctctgca tttacacaaa cacacacaca |
| 1201 | tgccacacag acacacatgc gcgcgcgcgc gcacacacac acacacacac acacacacac |
| 1261 | acacacacac caagtaccag tataggcatc tcccaactgc ttttccccat gtgtcctggt |
| 1321 | caagcccccc tcactctgtt tcctgttcag catgtactcc cctcatccga ttcccctcta |
| 1381 | tcagtcactg ccagttaata aacctttgca aacgtt |

SEQ ID NO: 27 Human SSX5 Amino Acid Sequence isoform 1 (NP_066295.3)

| | |
|---|---|
| 1 | mngddafvrr prvgsqipek mqkhpwrqvc drgihlvnls pfwkvgrepa ssikallcgr |
| 61 | gearafddia kyfsekewek mkasekiiyv ymkrkyeamt klgfkatlpp fmrnkrvadf |
| 121 | qgndfdndpn rgnqvehpqm tfgrlqgifp kitpekpaee gndskgvpea sgpqnngkql |
| 181 | rpsgklntse kvnktsgpkr gkhawthrvr erkqlviyee isdpqedde |

SEQ ID NO: 28 Human SSX5 cDNA Sequence variant 2 (NM_175723.1; CDS:
54 . . . 620)

| | |
|---|---|
| 1 | cgctctgatt gtttcgattc ttccaaaatc agagacagag tgctcccggt gccatgaacg |
| 61 | gagacgatgc ctttgtacgg agacctaggg ttggttctca aataccagag aagatgcaaa |
| 121 | aggccttcga tgatattgcc aaatacttct ctgagaaaga gtgggaaaag atgaaagcct |
| 181 | cggagaaaat catctatgtg tatatgaaga gaaagtatga ggccatgact aaactaggtt |
| 241 | tcaaggccac cctcccacct ttcatgcgta ataaacgggt cgcagacttc caggggaatg |
| 301 | attttgataa tgaccctaac cgtgggaatc aggttgaaca tcctcagatg actttcggca |
| 361 | ggctccaggg aatcttcccg aagatcacgc ccgagaagcc agcagaggaa ggaaatgatt |
| 421 | cgaagggagt gccagaagca tctggcccac agaacaatgg gaaacagctg cgcccctcag |
| 481 | gaaaactaaa tacctctgag aaggttaaca agacatctgg acccaaaagg gggaaacatg |
| 541 | cctggaccca cagagtgcgt gagagaaagc aactggtgat ttatgaagag atcagcgacc |
| 601 | ctcaggaaga tgacgagtaa ctcccctcgg ggatatgaca catgcccatg atgagaagca |
| 661 | gaacgtggtg acctttcacg aacatgggca tggctgcgga tccctcgtca tcaggtgtat |
| 721 | agcaagtgaa agcaagtgtt cacaacagtg aaaagttgag cgtcattttt cttagtgtgc |
| 781 | caagagttcg atgttagtgt ttctgttgta ttttgttaca gtgtgccatt ctgttagata |
| 841 | ttagcgtttt cactgatgag caagacatac ttaatgcata tttcagtttg tgtatccatg |
| 901 | cacctacctc agaaaacaag tatcgtcagg tattctctgc atagaacaac actaccctcc |
| 961 | tctcttccca gatgtgacca ctgagggcag ttctgagtgt ttaatttcag attttttcct |
| 1021 | ctgcatttac acaaacacac acacatgcca cacagacaca catgcgcgcg cgcgcgcaca |
| 1081 | cacacacaca cacacacaca cacacacaca cacaccaagt accagtatag gcatctccca |
| 1141 | actgcttttc cccatgtgtc ctggtcaagc ccccctcact ctgtttcctg ttcagcatgt |
| 1201 | actcccctca tccgattccc ctctatcagt cactgccagt taataaacct ttgcaaacgt |
| 1261 | taaaaaaaaa aaaaaa |

SEQ ID NO: 29 Human SSX5 Amino Acid Sequence isoform 2 (NP_783729.1)

| | |
|---|---|
| 1 | mngddafvrr prvgsqipek mqkafddiak yfsekewekm kasekiiyvy mkrkyeamtk |
| 61 | lgfkatlppf mrnkrvadfq gndfdndpnr gnqvehpqmt fgrlqgifpk itpekpaeeg |
| 121 | ndskgvpeas gpqnngkqlr psgklntsek vnktsgpkrg khawthrvre rkqlviyeei |
| 181 | sdpqedde |

SEQ ID NO: 30 Human SSX7 cDNA Sequence (NM_173358.2. CDS: 160-726)

| | |
|---|---|
| 1 | ccaggctcca gggacagaac cttctcaaag tgggggtgga gactctgatt ttcccgccta |
| 61 | aagcatcccc tgggattggc tactttaagt tcagagtatg catgctctga ctttctctct |
| 121 | cgattcttcc atactcagag tcagactgct cctggtgcca tgaacggaga cgacgccttt |
| 181 | gcaaggagac ctagggctgg tgctcaaata ccagagaaga tccaaaagtc cttcgatgat |
| 241 | attgccaaat acttctctaa gaaagagtgg gaaaagatga aatccttgga gaaaatcagc |
| 301 | tatgtgtata tgaagagaaa gtatgaggcc atgactaaac taggcttcaa ggccaccctc |
| 361 | ccacctttca tgcataatac aggggccaca gacctccagg ggaatgattt tgataatgac |
| 421 | cgtaaccaag ggaatcaggt tgaacgtcct cagatgactt tttgcaggct ccagagaatc |
| 481 | ttcccgaaga tcatgcccaa gaagccagca gaggaaggaa atgattcgaa gggagtgcca |
| 541 | gaagcatctg gctcacagaa cgatgggaaa cacctgtgcc ctccaggaaa accaagtacc |
| 601 | tctgagaaga ttaacaagac atccggaccc aaaaggggga aacatgcctg gacccacaga |
| 661 | ctgcgtgaga gaaagcagct ggtgatttat gaagagatca gcgaccctga agaagacgac |
| 721 | gagtaactcc cctcggggat acgacatatg cccatgatga gaagcagaac gtggtgacct |
| 781 | ttcacgaaca tgggcatggc tgcggacccc tcgtcatcag gtgcatagca agtgaaagca |
| 841 | agtgttcaca acagtgaaaa gttgagcgtc gtttttctta gtgtgacaag agttcgatgt |
| 901 | tagtgtttcc attgtatttt cttacagtgt gccattctgt tagatattag cgttttcatt |
| 961 | gatgagcaag acatgcttaa tgtgtatttc ggtttgtgta tccatgcacc tacctcagaa |
| 1021 | agcaagtata gtcaggtatt ctctccatag aacagcacta ccctcctctc tccccagatg |
| 1081 | tgactactga gggcagatct gagtgtttaa tttccgattt tcccctctgc atttacacac |
| 1141 | cagacacaca aacacacaca cacagacaca cacacacaca gacacaccaa gtaccagtat |
| 1201 | aagcatctcc catatgcttt tccccattgc catgagtcct ggtcaagccc cccttcaatt |
| 1261 | tgtttcctgt tcagcatgta ctcccctcct ctgattcccc gtatcagtca ctgacagtta |
| 1321 | atacaccttt gcaaacgttc |

SEQ ID NO: 31 Human SSX7 Amino Acid Sequence (NP_775494.1)

| | |
|---|---|
| 1 | mngddafarr pragaqipek iqksfddiak yfskkewekm kslekisyvy mkrkyeamtk |
| 61 | lgfkatlppf mhntgatdlq gndfdndrnq gnqverpqmt fcrlqrifpk impkkpaeeg |
| 121 | ndskgvpeas gsqndgkhlc ppgkpstsek inktsgpkrg khawthrlre rkqlviyeei |
| 181 | sdpeedde |

SEQ ID NO: 32 Human SS18 cDNA Sequence variant 1 (NM_001007559.2;
CDS: 79-1335)

| | |
|---|---|
| 1 | gagaggccgg cgtctctccc ccagtttgcc gttcacccgg agcgctcggg acttgccgat |
| 61 | agtggtgacg gcggcaacat gtctgtggct ttcgcggccc cgaggcagcg aggcaagggg |
| 121 | gagatcactc ccgctgcgat tcagaagatg ttggatgaca ataaccatct tattcagtgt |
| 181 | ataatggact ctcagaataa aggaaagacc tcagagtgtt ctcagtatca gcagatgttg |
| 241 | cacacaaact tggtatacct tgctacaata gcagattcta atcaaaatat gcagtctctt |
| 301 | ttaccagcac cacccacaca gaatatgcct atgggtcctg gagggatgaa tcagagcggc |
| 361 | cctcccccac ctccacgctc tcacaacatg ccttcagatg gaatggtagg tgggggtcct |
| 421 | cctgcaccgc acatgcagaa ccagatgaac ggccagatgc ctgggcctaa ccatatgcct |
| 481 | atgcagggac ctggacccaa tcaactcaat atgacaaaca gttccatgaa tatgccttca |
| 541 | agtagccatg gatccatggg aggttacaac cattctgtgc catcatcaca gagcatgcca |
| 601 | gtacagaatc agatgacaat gagtcaggga caaccaatgg gaaactatgg tcccagacca |
| 661 | aatatgagta tgcagccaaa ccaaggtcca atgatgcatc agcagcctcc ttctcagcaa |
| 721 | tacaatatgc cacagggagg cggacagcat taccaaggac agcagccacc tatgggaatg |
| 781 | atgggtcaag ttaaccaagg caatcatatg atgggtcaga gacagattcc tccctataga |
| 841 | cctcctcaac agggcccacc acagcagtac tcaggccagg aagactatta cggggaccaa |
| 901 | tacagtcatg gtggacaagg tcctccagaa ggcatgaacc agcaatatta ccctgatggt |
| 961 | cataatgatt acggttatca gcaaccgtcg tatcctgaac aaggctacga taggccttat |
| 1021 | gaggattcct cacaacatta ctacgaagga ggaaattcac agtatggcca acagcaagat |
| 1081 | gcataccagg gaccacctcc acaacaggga tatccacccc agcagcagca gtacccaggg |
| 1141 | cagcaaggtt acccaggaca gcagcagggc tacggtcctt cacagggtgg tccaggtcct |
| 1201 | cagtatccta actacccaca gggacaaggt cagcagtatg gaggatatag accaacacag |
| 1261 | cctggaccac cacagccacc ccagcagagg ccttatggat atgaccaggg acagtatgga |
| 1321 | aattaccagc agtgaaaaag tacttacatt ccagtagcca gtatctatta gcagccatat |
| 1381 | tgtcacctca gcactgtgga cacctccctg tgaagagatc cttccattcc atctagtttt |
| 1441 | tggaaaaacc ttgtggataa gtggctgttt catcagtaag cagcctttgt ggtttagtta |
| 1501 | taaaaggctt tagtagctca aaaatactct tgatttcaca tttctactct agatggcaac |
| 1561 | attggacaga aaatgcaatg acataaccaa tttgtaatga ttttggaact gtgtttcaaa |
| 1621 | tggactgtta cagactgaaa ggtgtgaaca gctttgtatg tttatgaagg gtaagggaat |
| 1681 | ttaatacttt tccacagatt tttttgtaag gggaagaggg aaatgtacac tttttacagc |
| 1741 | agcaatattt tgtatattat gtttatttca tgtggtgaat atgcaaggcg gtacactacg |
| 1801 | cactggacag catcagaaat cctctgttaa tgtggactgg aacatggtag atgcttgatt |
| 1861 | gttttggtct caaaatggtg tgctataaag ataaaggtga ggggaagaca aagcacacca |
| 1921 | tatgtccact gttctgttct catagaggaa attcaaatcc cttttatcta ttagataatc |
| 1981 | aagggcactg tgatacagtt ttgagtaaaa agacattttt taaaagcctt ccagttttgt |
| 2041 | ggattaaacc tttttataaa gatcatttat aatactgttt taaaatgtga ggcaataaga |
| 2101 | attactttgt gttggatctg aggaggcttt ggtaaaacag tttcatctaa atgaaagtgg |
| 2161 | taatcctctt ctaaaatagc aataactgaa aatgaaagtg ttaattttac cttgtttgag |
| 2221 | ttatcaggga acttagtaag taatatcaaa gcattttata aatgatatca aagaagagtc |
| 2281 | aacattgatc cagtcatttt attttgtaat attgagggat aattggttat taaactgaat |
| 2341 | agttcaggag actttacaaa cctttgtttc aactttctta tctggaaata atatcattta |
| 2401 | taaagggaca cttttatgtt tttccctttt ttatgttggt tgatataaca caaagagata |
| 2461 | tttaggaaaa tgcttattga tgaggtttat tctatctgtt tttaaagcac cgaggttgca |
| 2521 | ttctagataa ccttgtttat tagcatggca tattttaatc attatttgag actgtcctgt |
| 2581 | gcctgattat tttagctaaa ttcagggaga ttgcgtgggg caggaaagca tgcattgaaa |
| 2641 | aatttctaac cacggttatt taagcataat ctgaaaacat ctagcccaaa ggtaagttgc |
| 2701 | tattttcatc acagttgcct atgcccaggg aataagatgt attctttata attgaattgg |
| 2761 | tttttcccac gtctaactgg aaacaaaaca gaaggggcgt cataaatttg aataagcaga |
| 2821 | acatactgtt ctcaacatac tgtaatcaaa aggaggaatt tcagtgggtc tctgtgtgtg |
| 2881 | tatgagagag agagtgtgtg tttgtgtgtt tcaaggtcag aacaggtttt tttgtttttg |
| 2941 | ttttttgttc tttgtttttt tttttgagat ggagtcttgc tcttgtcgcc caggctggag |
| 3001 | tgcagtggcg caatctcagc tcactgcaac ctccgcctcc caggttcaag cagttctcct |
| 3061 | gcctcagcct cctgagtagc tgggatgaca ggcacccgcc accacaccca gctaattttt |
| 3121 | gtacttttag tagagacgag gtttcgccat gttggccagg ctggtctcga actcctgacc |
| 3181 | tcaggtgatc cacccgcctc ggccttccaa agtgctggga ttacaggcgt gagccaccgt |
| 3241 | gcctggccag aataggtttt ttctttcaac ttgatcagta gaaaatggac atcaagtttg |
| 3301 | aacagataaa tcatggacag ccttattgtg attgaaatgc ttgtaggttc tgtgccaatt |
| 3361 | ttccaccact gtgtactttg ttgctattta aaactgtatc aactctaacg gaagaataaa |
| 3421 | ttatttgtga ttttaaaaaa |

SEQ ID NO: 33 Human SS18 Amino Acid Sequence isoform 1 (NP_001007560.1)

| | |
|---|---|
| 1 | msvafaaprq rgkgeitpaa iqkmlddnnh liqcimdsqn kgktsecsqy qqmlhtnlvy |
| 61 | latiadsnqn mqsllpappt qnmpmgpggm nqsgpppppr shnmpsdgmv gggppaphmq |
| 121 | nqmngqmpgp nhmpmqgpgp nglnmtnssm nmpssshgsm ggynhsvpss qsmpvqnqmt |
| 181 | msqgqpmgny gprpnmsmqp nqgpmmhqqp psqqynmpqg ggqhyqgqqp pmgmmgqvnq |
| 241 | gnhmmgqrqi ppyrppqqgp pqqysgqedy ygdqyshggq gppegmnqqy ypdghndygy |
| 301 | qqpsypeggy drpyedssqh yyeggnsayg qqqdayqgpp pqqgyppqqq qypgqqgypg |
| 361 | qqqgygpsqg gpgpqypnyp qgqgqqyggy rptqpgppqp pqqrpygydq gqygnyqq |

SEQ ID NO: 34 Human SS18 cDNA Sequence variant 2 (NM_005637.3)

| | |
|---|---|
| 1 | gagaggccgg cgtctctccc ccagtttgcc gttcacccgg agcgctcggg acttgccgat |
| 61 | agtggtgacg gcggcaacat gtctgtggct ttcgcggccc cgaggcagcg aggcaagggg |
| 121 | gagatcactc ccgctgcgat tcagaagatg ttggatgaca ataaccatct tattcagtgt |
| 181 | ataatggact ctcagaataa aggaaagacc tcagagtgtt ctcagtatca gcagatgttg |
| 241 | cacacaaact tggtatacct tgctacaata gcagattcta atcaaaatat gcagtctctt |
| 301 | ttaccagcac cacccacaca gaatatgcct atgggtcctg gagggatgaa tcagagcggc |
| 361 | cctcccccac ctccacgctc tcacaacatg ccttcagatg gaatggtagg tgggggtcct |
| 421 | cctgcaccgc acatgcagaa ccagatgaac ggccagatgc ctgggcctaa ccatatgcct |
| 481 | atgcagggac ctggacccaa tcaactcaat atgacaaaca gttccatgaa tatgccttca |
| 541 | agtagccatg gatccatggg aggttacaac cattctgtgc catcatcaca gagcatgcca |
| 601 | gtacagaatc agatgacaat gagtcaggga caaccaatgg gaaactatgg tcccagacca |
| 661 | aatatgagta tgcagccaaa ccaaggtcca atgatgcatc agcagcctcc ttctcagcaa |
| 721 | tacaatatgc cacagggagg cggacagcat taccaaggac agcagccacc tatgggaatg |
| 781 | atgggtcaag ttaaccaagg caatcatatg atgggtcaga gacagattcc tccctataga |
| 841 | cctcctcaac agggcccacc acagcagtac tcaggccagg aagactatta cggggaccaa |
| 901 | tacagtcatg gtggacaagg tcctccagaa ggcatgaacc agcaatatta ccctgatgga |
| 961 | aattcacagt atggccaaca gcaagatgca taccagggac cacctccaca acagggatat |
| 1021 | ccaccccagc agcagcagta cccagggcag caaggttacc caggacagca gcagggctac |
| 1081 | ggtccttcac agggtggtcc aggtcctcag tatcctaact acccacaggg acaaggtcag |
| 1141 | cagtatggag gatatagacc aacacagcct ggaccaccac agccacccca gcagaggcct |
| 1201 | tatggatatg accagggaca gtatggaaat taccagcagt gaaaaagtac ttacattcca |
| 1261 | gtagccagta tctattagca gccatattgt cacctcagca ctgtggacac ctccctgtga |
| 1321 | agagatcctt ccattccatc tagtttttgg aaaaaccttg tggataagtg gctgtttcat |
| 1381 | cagtaagcag cctttgtggt ttagttataa aaggctttag tagctcaaaa atactcttga |
| 1441 | tttcacattt ctactctaga tggcaacatt ggacagaaaa tgcaatgaca taaccaattt |
| 1501 | gtaatgattt tggaactgtg tttcaaatgg actgttacag actgaaaggt gtgaacagct |
| 1561 | ttgtatgttt atgaagggta agggaattta atacttttcc acagattttt ttgtaagggg |
| 1621 | aagagggaaa tgtacacttt ttacagcagc aatattttgt atattatgtt tatttcatgt |
| 1681 | ggtgaatatg caaggcggta cactacgcac tggacagcat cagaaatcct ctgttaatgt |
| 1741 | ggactggaac atggtagatg cttgattgtt ttggtctcaa aatggtgtgc tataaagata |
| 1801 | aaggtgaggg gaagacaaag cacaccatat gtccactgtt ctgttctcat agaggaaatt |
| 1861 | caaatccctt ttatctatta gataatcaag ggcactgtga tacagttttg agtaaaaaga |
| 1921 | cattttttaa aagccttcca gttttgtgga ttaaaccttt ttataaagat catttataat |
| 1981 | actgttttaa aatgtgaggc aataagaatt actttgtgtt ggatctgagg aggctttggt |
| 2041 | aaaacagttt catctaaatg aaagtggtaa tcctcttcta aaatagcaat aactgaaaat |
| 2101 | gaaagtgtta attttacctt gtttgagtta tcagggaact tagtaagtaa tatcaaagca |
| 2161 | ttttataaat gatatcaaag aagagtcaac attgatccag tcattttatt ttgtaatatt |
| 2221 | gagggataat tggttattaa actgaatagt tcaggagact ttacaaacct ttgtttcaac |
| 2281 | tttcttatct ggaaataata tcatttataa agggacactt ttatgttttt ccctttttta |
| 2341 | tgttggttga tataacacaa agagatattt aggaaaatgc ttattgatga ggtttattct |
| 2401 | atctgttttt aaagcaccga ggttgcattc tagataacct tgtttattag catggcatat |
| 2461 | tttaatcatt atttgagact gtcctgtgcc tgattatttt agctaaattc agggagattg |
| 2521 | cgtggggcag gaaagcatgc attgaaaaat ttctaaccac ggttatttaa gcataatctg |
| 2581 | aaaacatcta gcccaaaggt aagttgctat tttcatcaca gttgcctatg cccagggaat |
| 2641 | aagatgtatt ctttataatt gaattggttt ttcccacgtc taactggaaa caaaacagaa |
| 2701 | ggggcgtcat aaatttgaat aagcagaaca tactgttctc aacatactgt aatcaaaagg |
| 2761 | aggaatttca gtgggtctct gtgtgtgtat gagagagaga gtgtgtgttt gtgtgtttca |
| 2821 | aggtcagaac aggttttttt gtttttgttt tttgttcttt gttttttttt ttgagatgga |
| 2881 | gtcttgctct tgtcgcccag gctggagtgc agtggcgcaa tctcagctca ctgcaacctc |
| 2941 | cgcctcccag gttcaagcag ttctcctgcc tcagcctect gagtagctgg gatgacaggc |
| 3001 | acccgccacc acacccagct aatttttgta cttttagtag agacgaggtt tcgccatgtt |
| 3061 | ggccaggctg gtctcgaact cctgacctca ggtgatccac ccgcctcggc cttccaaagt |
| 3121 | gctgggatta caggcgtgag ccaccgtgcc tggccagaat aggttttttc tttcaacttg |
| 3181 | atcagtagaa aatggacatc aagtttgaac agataaatca tggacagcct tattgtgatt |
| 3241 | gaaatgcttg taggttctgt gccaattttc caccactgtg tactttgttg ctatttaaaa |
| 3301 | ctgtatcaac tctaacggaa gaataaatta tttgtgattt taaaaaa |

SEQ ID NO: 35 Human SS18 Amino Acid Sequence isoform 2 (NP_005628.2)

| | |
|---|---|
| 1 | msvafaaprq rgkgeitpaa iqkmlddnnh liqcimdsqn kgktsecsqy qqmlhtnlvy |
| 61 | latiadsnqn mqsllpappt qnmpmgpggm nqsgpppppr shnmpsdgmv gggppaphmq |
| 121 | nqmngqmpgp nhmpmqgpgp nqlnmtnssm nmpssshgsm ggynhsvpss qsmpvqnqmt |
| 181 | msqgqpmgny gprpnmsmqp nqgpmmhqqp psqqynmpqg ggqhyqgqqp pmgmmgqvnq |
| 241 | gnhmmgqrqi ppyrppqqgp pqqysgqedy ygdqyshggq gppegmnqqy ypdgnsqygq |
| 301 | qqdayqgppp qqgyppqqqq ypgqqgypgq qqgygpsqgg pgpqypnypq gqgqqyggyr |
| 361 | ptqpgppqpp qqrpygydqg qygnyqq |

SEQ ID NO: 36 Human SS18 cDNA Sequence variant 3 (NM_001308201.1; CDS:
123-1310)

| | |
|---|---|
| 1 | ccttccacct ctgccctatc tcggcagatg ctccacggat ttgcacgaac tcccgagtct |
| 61 | tgacctccct cccctctccg ggctgccggg acaactcggg gcggccactc ttgccaggag |
| 121 | gcatgttgga tgacaataac catcttattc agtgtataat ggactctcag aataaaggaa |
| 181 | agacctcaga gtgttctcag tatcagcaga tgttgcacac aaacttggta taccttgcta |
| 241 | caatagcaga ttctaatcaa aatatgcagt ctcttttacc agcaccaccc acacagaata |
| 301 | tgcctatggg tcctggaggg atgaatcaga gcggccctcc cccacctcca cgctctcaca |
| 361 | acatgccttc agatggaatg gtaggtgggg gtcctcctgc accgcacatg cagaaccaga |
| 421 | tgaacggcca gatgcctggg cctaaccata tgcctatgca gggacctgga cccaatcaac |
| 481 | tcaatatgac aaacagttcc atgaatatgc cttcaagtag ccatggatcc atgggaggtt |
| 541 | acaaccattc tgtgccatca tcacagagca tgccagtaca gaatcagatg acaatgagtc |
| 601 | agggacaacc aatgggaaac tatggtccca gaccaaatat gagtatgcag ccaaaccaag |
| 661 | gtccaatgat gcatcagcag cctccttctc agcaatacaa tatgccacag ggaggcggac |
| 721 | agcattacca aggacagcag ccacctatgg gaatgatggg tcaagttaac caaggcaatc |
| 781 | atatgatggg tcagagacag attcctccct atagacctcc tcaacagggc ccaccacagc |
| 841 | agtactcagg ccaggaagac tattacgggg accaatacag tcatggtgga caaggtcctc |
| 901 | cagaaggcat gaaccagcaa tattaccctg atggtcataa tgattacggt tatcagcaac |
| 961 | cgtcgtatcc tgaacaaggc tacgataggc cttatgagga ttcctcacaa cattactacg |
| 1021 | aaggaggaaa ttcacagtat ggccaacagc aagatgcata ccagggacca cctccacaac |
| 1081 | agggatatcc accccagcag cagcagtacc cagggcagca aggttaccca ggacagcagc |
| 1141 | agggctacgg tccttcacag ggtggtccag gtcctcagta tcctaactac ccacagggac |
| 1201 | aaggtcagca gtatggagga tatagaccaa cacagcctgg accaccacag ccaccccagc |
| 1261 | agaggcctta tggatatgac cagggacagt atggaaatta ccagcagtga aaaagtactt |
| 1321 | acattccagt agccagtatc tattagcagc catattgtca cctcagcact gtggacacct |
| 1381 | ccctgtgaag agatccttcc attccatcta gtttttggaa aaaccttgtg gataagtggc |
| 1441 | tgtttcatca gtaagcagcc tttgtggttt agttataaaa ggctttagta gctcaaaaat |
| 1501 | actcttgatt tcacatttct actctagatg gcaacattgg acagaaaatg caatgacata |
| 1561 | accaatttgt aatgattttg gaactgtgtt tcaaatggac tgttacagac tgaaaggtgt |
| 1621 | gaacagcttt gtatgtttat gaagggtaag ggaatttaat acttttccac agattttttt |
| 1681 | gtaaggggaa gagggaaatg tacacttttt acagcagcaa tattttgtat attatgttta |
| 1741 | tttcatgtgg tgaatatgca aggcggtaca ctacgcactg gacagcatca gaaatcctct |
| 1801 | gttaatgtgg actggaacat ggtagatgct tgattgtttt ggtctcaaaa tggtgtgcta |
| 1861 | taaagataaa ggtgagggga agacaaagca caccatatgt ccactgttct gttctcatag |
| 1921 | aggaaattca aatccctttt atctattaga taatcaaggg cactgtgata cagttttgag |
| 1981 | taaaaagaca ttttttaaaa gccttccagt tttgtggatt aaaccttttt ataaagatca |
| 2041 | tttataatac tgttttaaaa tgtgaggcaa taagaattac tttgtgttgg atctgaggag |
| 2101 | gctttggtaa aacagtttca tctaaatgaa agtggtaatc ctcttctaaa atagcaataa |
| 2161 | ctgaaaatga aagtgttaat tttaccttgt ttgagttatc agggaactta gtaagtaata |
| 2221 | tcaaagcatt ttataaatga tatcaaagaa gagtcaacat tgatccagtc attttatttt |
| 2281 | gtaatattga gggataattg gttattaaac tgaatagttc aggagacttt acaaaccttt |
| 2341 | gtttcaactt tcttatctgg aaataatatc atttataaag ggacactttt atgtttttcc |
| 2401 | cttttttatg ttggttgata taacacaaag agatatttag gaaaatgctt attgatgagg |
| 2461 | tttattctat ctgtttttaa agcaccgagg ttgcattcta gataaccttg tttattagca |
| 2521 | tggcatattt taatcattat ttgagactgt cctgtgcctg attattttag ctaaattcag |
| 2581 | ggagattgcg tggggcagga aagcatgcat tgaaaaattt ctaaccacgg ttatttaagc |
| 2641 | ataatctgaa aacatctagc ccaaaggtaa gttgctattt tcatcacagt tgcctatgcc |
| 2701 | cagggaataa gatgtattct ttataattga attggttttt cccacgtcta actggaaaca |
| 2761 | aaacagaagg ggcgtcataa atttgaataa gcagaacata ctgttctcaa catactgtaa |
| 2821 | tcaaaaggag gaatttcagt gggtctctgt gtgtgtatga gagagagagt gtgtgtttgt |
| 2881 | gtgtttcaag gtcagaacag gtttttttgt ttttgttttt tgttctttgt tttttttttt |
| 2941 | gagatggagt cttgctcttg tcgcccaggc tggagtgcag tggcgcaatc tcagctcact |
| 3001 | gcaacctccg cctcccaggt tcaagcagtt ctcctgcctc agcctcctga gtagctggga |
| 3061 | tgacaggcac ccgccaccac acccagctaa tttttgtact tttagtagag acgaggtttc |
| 3121 | gccatgttgg ccaggctggt ctcgaactcc tgacctcagg tgatccaccc gcctcggcct |
| 3181 | tccaaagtgc tgggattaca ggcgtgagcc accgtgcctg gccagaatag gttttttctt |
| 3241 | tcaacttgat cagtagaaaa tggacatcaa gtttgaacag ataaatcatg gacagcctta |
| 3301 | ttgtgattga aatgcttgta ggttctgtgc caattttcca ccactgtgta ctttgttgct |
| 3361 | atttaaaact gtatcaactc taacggaaga ataaattatt tgtgatttta aaaaa |

SEQ ID NO: 37 Human SS18 Amino Sequence isoform 3 (NP_001295130.1)

| | |
|---|---|
| 1 | mlddnnhliq cimdsqnkgk tsecsqyqqm lhtnlvylat iadsnqnmqs llpapptqnm |
| 61 | pmgpggmnqs gppppprshn mpsdgmvggg ppaphmqnqm ngqmpgpnhm pmqgpgpnql |
| 121 | nmtnssmnmp ssshgsmggy nhsvpssqsm pvqnqmtmsq gqpmgnygpr pnmsmqpnqg |
| 181 | pmmhqqppsq qynmpqgggq hyqgqqppmg mmgqvnqgnh mmgqrqippy rppqqgppqq |
| 241 | ysgqedyygd qyshggqgpp egmnqqyypd ghndygyqqp sypeqgydrp yedssqhyye |
| 301 | ggnsqygqqq dayqgpppqq gyppqqqqyp gqqgypgqqq gygpsqggpg pqypnypqqq |
| 361 | gqqyggyrpt qpgppqppqq rpygydqgqy gnyqq |

SEQ ID NO: 38 Mouse SS18 Amino Acid Sequence isoform 1 (NP_033306.2)

| | |
|---|---|
| 1 | msvafaaprq rgkgeitpaa iqkmldennh liqcimdygn kgkasecsqy qqilhtnlvy |
| 61 | latiadsnqn mqsllpappt qtmpmgpggm sqsgpppppr shnmpsdgmv gggppaphmq |
| 121 | nqmngqmpgp nhmpmqgpgp sqlsmtnssm nmpssshgsm ggynhsvpss qsmpvqnqmt |
| 181 | msqgqpmgny gprpnmnmqp nqgpmmhqqp psqqynmppg gaqhyqgqqa pmglmgqvnq |
| 241 | gshmmgqrqm ppyrppqqgp pqqysgqedy ygdqyshggq gppegmnqqy ypdghndygy |
| 301 | qqpsypeqgy drpyedssqh yyeggnsqyg qqqdayqgpp pqqgyppqqq qypgqqgypg |
| 361 | qqqsygpsqg gpgpqypnyp qgqgqqyggy rptqpgppqp pqqrpygydq gqygnyqq |

SEQ ID NO: 39 Mouse SS18 cDNA Sequence variant 1 (NM_009280.2; CDS: 180-
1436)

| | |
|---|---|
| 1 | ccttgctggg agctgcggct cagcgttaag gccaagccgg ccagcgaggg acgcggcccg |
| 61 | ggagcatcct ccccccaccg cgcgccctaa ggtggaactg cccggaggcg ggcgtcgggc |
| 121 | ccccagctcc gcgggccctg gagcgctcgg gactcgctga tcgcgggctc ggcggcaaca |
| 181 | tgtctgtggc gttcgcagcc ccgaggcagc ggggcaaggg cgaaatcacg cccgccgcca |
| 241 | tccagaagat gctggatgaa aacaaccatc ttattcagtg tataatggac tatcagaaca |
| 301 | aagggaaggc ctcggagtgc tcgcagtatc agcagatatt gcatacaaac ctggtatacc |
| 361 | ttgctacaat agcagactct aatcaaaata tgcagtctct cttaccagca ccgcccacac |
| 421 | agactatgcc aatgggtcct ggagggatga gtcagagtgg ccctccaccc cctccccgct |
| 481 | ctcacaacat gccttcagat ggaatggtgg gtgggggccc tcctgcacca cacatgcaga |
| 541 | accagatgaa cggccagatg cctgggccta accatatgcc aatgcaggga cctggaccca |
| 601 | gtcagctcag catgacaaac agctccatga atatgccttc aagtagccat ggctccatgg |
| 661 | gaggttacaa ccattctgtg ccgtcatccc agagcatgcc cgtgcagaac cagatgacaa |
| 721 | tgagtcaggg gcagccaatg ggaaactatg gtcccagacc aaacatgaat atgcaaccaa |
| 781 | atcaagggcc gatgatgcac cagcagcctc cttctcagca gtacaatatg ccacctggag |
| 841 | gggcacagca ttaccaagga cagcaggcgc ccatggggct gatgggccaa gttaaccaag |
| 901 | gcagtcacat gatgggccag cgacagatgc ctccctacag acctccgcaa cagggcccac |
| 961 | cacagcagta ctcaggccag gaagactatt atggggacca atacagtcat ggtggacaag |
| 1021 | gtcctccaga aggcatgaac cagcaatatt accctgatgg tcataatgat tacggttatc |
| 1081 | agcaaccgtc gtatcctgaa caaggctacg ataggcctta tgaggattcc tcacaacatt |
| 1141 | actacgaagg aggaaactcc cagtatggcc aacagcaaga cgcttaccag ggaccacctc |
| 1201 | cacagcaagg atacccaccc cagcagcagc agtacccggg acagcaggga tacccagggc |
| 1261 | agcagcagag ctatggtcct tcgcagggcg gtccaggtcc tcagtatcct aattatcctc |
| 1321 | agggtcaagg tcagcagtat gggggctata gaccaacaca gccaggacca ccccagccac |
| 1381 | cccagcagag gccttatggg tacgaccagg gacagtatgg aaattaccag cagtgaaaat |
| 1441 | gtccttacat tccaatagcc agtacctatt agcaggcacg ttgtcacagc actgcaccat |
| 1501 | ggacaccccc ctgggaagac tccttccatt ccagctaggt ttttgggaaa acctttggct |
| 1561 | aagtggctgc ttcgtcagca agtagctgtt atggtttagt ttgtaaaggc ttcgtagcta |
| 1621 | ccgatgcacc tgatttcacg tttctactct agatggcaac attggacaga aaatgcattg |
| 1681 | acgtgaggag tttgcagcgg tttcagaact gtgctgcaaa tggactgtca cagcctgaaa |
| 1741 | ggtgtgagca gctgggtgtg tgttcgcgga gcttcagggg gtttcatact tttccaccga |
| 1801 | ttattttgta aggggaaggg ggaaatgtac actttttaca gcagcaatat tttgtctatt |
| 1861 | atgtttattt catgtgataa atatgcaaag cggtacacta cacactgggc agaatcagaa |
| 1921 | cccctgttaa tgtggagtgt ggtagatgct cggtgctgtg gtgctctgaa gacaggcgag |
| 1981 | gggaggcaga agcccaccac aggcccgctg ttagttctta gaggaaactc ctctctctct |
| 2041 | tatctaccag attagcaagg gcgctgtgat acagtttttt gagtacaaag acatttttta |
| 2101 | aaaagccttc cagttttgtg cattaaaacc tttttgtaaa tatggtttat aatactgttt |
| 2161 | tcaaacgcaa ggcaataatt atgttgcatc tgtgaacttt ggcaggtttg tgtaaaagga |
| 2221 | gggaagcctc tcttaaaaca gcaataacag aaaaggagga agcgggatgt ttttaccttg |
| 2281 | tcttgtaatc agggagctct caccacgtca gagaggaggc agcattggtc tcaccttact |
| 2341 | gttttttaca ttaccatgat tggttcatgg agcagggagg agtccacgag acttcacacg |
| 2401 | cttgtgcttt aactttctta actgggcaca agcaaagggc gccttcgtgt tcctctcttc |
| 2461 | atcttagtta atgcgcgagg aaaatgcttt gatggccatt tctcattcgc actgaaagcc |
| 2521 | gagaggtgac attttacggt ttcttgtttt taagcacgac atacttaatc attatttgag |
| 2581 | actgattatt ttagctaaat ttggggatat gccatggggc aagaaaacat gtactgagag |
| 2641 | atttctaaac acatctattt aagcatactt taaaaatatc tagcccaaag gtaagttgct |
| 2701 | gtatcctcac agttgtctgc atccagggaa tatgactgaa tataacatat ctttgtaatt |
| 2761 | gaattagttt ttgccacttc taactgaaaa cagaacagaa ggagtgccat aaatgcaaag |
| 2821 | aagcaaagtg tactgttgtc aacatactgt aatcagagga ggggtttcaa tgtgtctgga |
| 2881 | tgagagtgtg tgtgtttaag gtcagagtat agggtgttct tcaacttgga cagtagaaaa |
| 2941 | taggcatcaa gtgtgaaccg gtgaggcgtg gacagccttc ttgtgactga gatgcttgta |
| 3001 | agttctgtgc caggttctcc accactgtgt actttattgc tatttaaaac tgtatcaact |
| 3061 | ctaacgaaag aataaattat ttgtgatttt aaaaaaaaaa aaaaaaaaaa |

SEQ ID NO: 40 Mouse SS18 Amino Acid Sequence isoform 2 (NP_001154841.1)

| | |
|---|---|
| 1 | msvafaaprq rgkgeitpaa iqkmldennh liqcimdyqn kgkasecsqy qqilhtnlvy |
| 61 | latiadsnqn mqsllpappt qtmpmgpggm sqsgpppppr shnmpsdgmv gggppaphmq |
| 121 | nqmngqmpgp nhmpmqgpgp sqlsmtnssm nmpssshgsm ggynhsvpss qsmpvqnqmt |
| 181 | msqgqpmgny gprpnmnmqp nqgpmmhqqp psqqynmppg gaqhyqgqqa pmglmgqvnq |
| 241 | gshmmgqrqm ppyrppqqgp pqqysgqedy ygdqyshggq gppegmnqqy ypdgnsqygq |
| 301 | qqdayqgppp qqgyppqqqq ypgqqgypgq qqsygpsqgg pgpqypnypq gqgqqyggyr |
| 361 | ptqpgppqpp qqrpygydqg qygnyqq |

SEQ ID NO: 41 Mouse SS18 cDNA Sequence variant 2 (NM_001161369.1; CDS:
180-1343)

| | |
|---|---|
| 1 | ccttgctggg agctgcggct cagcgttaag gccaagccgg ccagcgaggg acgcggcccg |
| 61 | ggagcatcct ccccccaccg cgcgccctaa ggtggaactg cccggaggcg ggcgtcgggc |
| 121 | ccccagctcc gcgggccctg gagcgctcgg gactcgctga tcgcgggctc ggcggcaaca |
| 181 | tgtctgtggc gttcgcagcc ccgaggcagc ggggcaaggg cgaaatcacg cccgccgcca |
| 241 | tccagaagat gctggatgaa aacaaccatc ttattcagtg tataatggac tatcagaaca |
| 301 | aagggaaggc ctcggagtgc tcgcagtatc agcagatatt gcatacaaac ctggtatacc |
| 361 | ttgctacaat agcagactct aatcaaaata tgcagtctct cttaccagca ccgcccacac |
| 421 | agactatgcc aatgggtcct ggagggatga gtcagagtgg ccctccaccc cctccccgct |
| 481 | ctcacaacat gccttcagat ggaatggtgg gtgggggccc tcctgcacca cacatgcaga |
| 541 | accagatgaa cggccagatg cctgggccta accatatgcc aatgcaggga cctggaccca |
| 601 | gtcagctcag catgacaaac agctccatga atatgccttc aagtagccat ggctccatgg |
| 661 | gaggttacaa ccattctgtg ccgtcatccc agagcatgcc cgtgcagaac cagatgacaa |
| 721 | tgagtcaggg gcagccaatg ggaaactatg gtcccagacc aaacatgaat atgcaaccaa |
| 781 | atcaagggcc gatgatgcac cagcagcctc cttctcagca gtacaatatg ccacctggag |
| 841 | gggcacagca ttaccaagga cagcaggcgc ccatggggct gatgggccaa gttaaccaag |
| 901 | gcagtcacat gatgggccag cgacagatgc ctccctacag acctccgcaa cagggcccac |
| 961 | cacagcagta ctcaggccag gaagactatt atggggacca atacagtcat ggtggacaag |
| 1021 | gtcctccaga aggcatgaac cagcaatatt accctgatgg aaactcccag tatggccaac |
| 1081 | agcaagacgc ttaccaggga ccacctccac agcaaggata cccaccccag cagcagcagt |
| 1141 | acccgggaca gcagggatac ccagggcagc agcagagcta tggtccttcg cagggcggtc |
| 1201 | caggtcctca gtatcctaat tatcctcagg gtcaaggtca gcagtatggg ggctatagac |
| 1261 | caacacagcc aggaccaccc cagccacccc agcagaggcc ttatgggtac gaccagggac |
| 1321 | agtatggaaa ttaccagcag tgaaaatgtc cttacattcc aatagccagt acctattagc |
| 1381 | aggcacgttg tcacagcact gcaccatgga cacccccctg ggaagactcc ttccattcca |
| 1441 | gctaggtttt tgggaaaacc tttggctaag tggctgcttc gtcagcaagt agctgttatg |
| 1501 | gtttagtttg taaaggcttc gtagctaccg atgcacctga tttcacgttt ctactctaga |
| 1561 | tggcaacatt ggacagaaaa tgcattgacg tgaggagttt gcagcggttt cagaactgtg |
| 1621 | ctgcaaatgg actgtcacag cctgaaaggt gtgagcagct gggtgtgtgt tcgcggagct |
| 1681 | tcagggggtt tcatactttt ccaccgatta ttttgtaagg ggaaggggga aatgtacact |
| 1741 | ttttacagca gcaatatttt gtctattatg tttatttcat gtgataaata tgcaaagcgg |
| 1801 | tacactacac actgggcaga atcagaaccc ctgttaatgt ggagtgtggt agatgctcgg |
| 1861 | tgctgtggtg ctctgaagac aggcgagggg aggcagaagc ccaccacagg cccgctgtta |
| 1921 | gttcttagag gaaactcctc tctctcttat ctaccagatt agcaagggcg ctgtgataca |
| 1981 | gttttttgag tacaaagaca ttttttaaaa agccttccag ttttgtgcat taaaaccttt |
| 2041 | ttgtaaatat ggtttataat actgttttca aacgcaaggc aataattatg ttgcatctgt |
| 2101 | gaactttggc aggtttgtgt aaaaggaggg aagcctctct taaaacagca ataacagaaa |
| 2161 | aggaggaagc gggatgtttt taccttgtct tgtaatcagg gagctctcac cacgtcagag |
| 2221 | aggaggcagc attggtctca ccttactgtt ttttacatta ccatgattgg ttcatggagc |
| 2281 | agggaggagt ccacgagact tcacacgctt gtgctttaac tttcttaact gggcacaagc |
| 2341 | aaagggcgcc ttcgtgttcc tctcttcatc ttagttaatg cgcgaggaaa atgctttgat |
| 2401 | ggccatttct cattcgcact gaaagccgag aggtgacatt ttacggtttc ttgtttttaa |
| 2461 | gcacgacata cttaatcatt atttgagact gattatttta gctaaatttg gggatatgcc |
| 2521 | atggggcaag aaaacatgta ctgagagatt tctaaacaca tctatttaag catactttaa |
| 2581 | aaatatctag cccaaaggta agttgctgta tcctcacagt tgtctgcatc cagggaatat |
| 2641 | gactgaatat aacatatctt tgtaattgaa ttagtttttg ccacttctaa ctgaaaacag |
| 2701 | aacagaagga gtgccataaa tgcaaagaag caaagtgtac tgttgtcaac atactgtaat |
| 2761 | cagaggaggg gtttcaatgt gtctggatga gagtgtgtgt gtttaaggtc agagtatagg |
| 2821 | gtgttcttca acttggacag tagaaaatag gcatcaagtg tgaaccggtg aggcgtggac |
| 2881 | agccttcttg tgactgagat gcttgtaagt tctgtgccag gttctccacc actgtgtact |
| 2941 | ttattgctat ttaaaactgt atcaactcta acgaaagaat aaattatttg tgattttaaa |
| 3001 | aaaaaaaaaa aaaaaaa |

SEQ ID NO: 42 Mouse SS18 Amino Acid Sequence isoform 3 (NP_001154842.1)

| | |
|---|---|
| 1 | msvafaaprq rgkgeitpaa iqkmldennh liqcimdyqn kgkasecsqy qqilhtnlvy |
| 61 | latiadsnqn mqsllpappt qtmpmgpggm sqsgpppppr shnmpsdgmv gggppaphmq |
| 121 | nqmngqmpgp mmhqqppsqq ynmppggaqh yqgqqapmgl mgqvnqgshm mgqrqmppyr |
| 181 | ppqqgppqqy sgqedyygdq yshggqgppe gmnqqyypdg hndygyqqps ypeqgydrpy |
| 241 | edssqhyyeg gnsqygqqqd ayqgpppqqg yppqqqqypg qqgypgqqqs ygpsqggpgp |
| 301 | qypnypqgqg qqyggyrptq pgppqppqqr pygydqgqyg nyqq |

SEQ ID NO: 43 Mouse SS18 cDNA Sequence variant 3 (NM_001161370.1; CDS:
180-1214)

| | |
|---|---|
| 1 | ccttgctggg agctgcggct cagcgttaag gccaagccgg ccagcgaggg acgcggcccg |
| 61 | ggagcatcct ccccccaccg cgcgccctaa ggtggaactg cccggaggcg ggcgtcgggc |
| 121 | ccccagctcc gcgggccctg gagcgctcgg gactcgctga tcgcgggctc ggcggcaaca |
| 181 | tgtctgtggc gttcgcagcc ccgaggcagc ggggcaaggg cgaaatcacg cccgccgcca |
| 241 | tccagaagat gctggatgaa aacaaccatc ttattcagtg tataatggac tatcagaaca |
| 301 | aagggaaggc ctcggagtgc tcgcagtatc agcagatatt gcatacaaac ctggtatacc |
| 361 | ttgctacaat agcagactct aatcaaaata tgcagtctct cttaccagca ccgcccacac |
| 421 | agactatgcc aatgggtcct ggagggatga gtcagagtgg ccctccaccc cctccccgct |
| 481 | ctcacaacat gccttcagat ggaatggtgg gtgggggccc tcctgcacca cacatgcaga |
| 541 | accagatgaa cggccagatg cctgggccga tgatgcacca gcagcctcct tctcagcagt |
| 601 | acaatatgcc acctggaggg gcacagcatt accaaggaca gcaggcgccc atggggctga |
| 661 | tgggccaagt taaccaaggc agtcacatga tgggccagcg acagatgcct ccctacagac |
| 721 | ctccgcaaca gggcccacca cagcagtact caggccagga agactattat ggggaccaat |
| 781 | acagtcatgg tggacaaggt cctccagaag gcatgaacca gcaatattac cctgatggtc |
| 841 | ataatgatta cggttatcag caaccgtcgt atcctgaaca aggctacgat aggccttatg |
| 901 | aggattcctc acaacattac tacgaaggag gaaactccca gtatggccaa cagcaagacg |
| 961 | cttaccaggg accacctcca cagcaaggat acccacccca gcagcagcag tacccgggac |
| 1021 | agcagggata cccagggcag cagcagagct atggtccttc gcagggcggt ccaggtcctc |
| 1081 | agtatcctaa ttatcctcag ggtcaaggtc agcagtatgg gggctataga ccaacacagc |
| 1141 | caggaccacc ccagccaccc cagcagaggc cttatgggta cgaccaggga cagtatggaa |
| 1201 | attaccagca gtgaaaatgt ccttacattc caatagccag tacctattag caggcacgtt |
| 1261 | gtcacagcac tgcaccatgg acacccccct gggaagactc cttccattcc agctaggttt |
| 1321 | ttgggaaaac ctttggctaa gtggctgctt cgtcagcaag tagctgttat ggtttagttt |
| 1381 | gtaaaggctt cgtagctacc gatgcacctg atttcacgtt tctactctag atggcaacat |
| 1441 | tggacagaaa atgcattgac gtgaggagtt tgcagcggtt tcagaactgt gctgcaaatg |
| 1501 | gactgtcaca gcctgaaagg tgtgagcagc tgggtgtgtg ttcgcggagc ttcagggggt |
| 1561 | ttcatacttt tccaccgatt attttgtaag gggaaggggg aaatgtacac tttttacagc |
| 1621 | agcaatattt tgtctattat gtttatttca tgtgataaat atgcaaagcg gtacactaca |
| 1681 | cactgggcag aatcagaacc cctgttaatg tggagtgtgg tagatgctcg gtgctgtggt |
| 1741 | gctctgaaga caggcgaggg gaggcagaag cccaccacag gcccgctgtt agttcttaga |
| 1801 | ggaaactcct ctctctctta tctaccagat tagcaagggc gctgtgatac agttttttga |
| 1861 | gtacaaagac attttttaaa aagccttcca gttttgtgca ttaaaacctt tttgtaaata |
| 1921 | tggtttataa tactgttttc aaacgcaagg caataattat gttgcatctg tgaactttgg |
| 1981 | caggtttgtg taaaaggagg gaagcctctc ttaaaacagc aataacagaa aaggaggaag |
| 2041 | cgggatgttt ttaccttgtc ttgtaatcag ggagctctca ccacgtcaga gaggaggcag |
| 2101 | cattggtctc accttactgt tttttacatt accatgattg gttcatggag cagggaggag |
| 2161 | tccacgagac ttcacacgct tgtgctttaa ctttcttaac tgggcacaag caaagggcgc |
| 2221 | cttcgtgttc ctctcttcat cttagttaat gcgcgaggaa aatgctttga tggccatttc |
| 2281 | tcattcgcac tgaaagccga gaggtgacat tttacggttt cttgttttta agcacgacat |
| 2341 | acttaatcat tatttgagac tgattatttt agctaaattt ggggatatgc catggggcaa |
| 2401 | gaaaacatgt actgagagat ttctaaacac atctatttaa gcatacttta aaaatatcta |
| 2461 | gcccaaaggt aagttgctgt atcctcacag ttgtctgcat ccagggaata tgactgaata |
| 2521 | taacatatct ttgtaattga attagttttt gccacttcta actgaaaaca gaacagaagg |
| 2581 | agtgccataa atgcaaagaa gcaaagtgta ctgttgtcaa catactgtaa tcagaggagg |
| 2641 | ggtttcaatg tgtctggatg agagtgtgtg tgtttaaggt cagagtatag ggtgttcttc |
| 2701 | aacttggaca gtagaaaata ggcatcaagt gtgaaccggt gaggcgtgga cagccttctt |
| 2761 | gtgactgaga tgcttgtaag ttctgtgcca ggttctccac cactgtgtac tttattgcta |
| 2821 | tttaaaactg tatcaactct aacgaaagaa taaattattt gtgattttaa aaaaaaaaaa |
| 2881 | aaaaaaaa |

SEQ ID NO: 44 Mouse SS18 Amino Acid Sequence isoform 4 (NP_001154843.1)

| | |
|---|---|
| 1 | msvafaaprq rgkgeitpaa iqkmldennh liqcimdygn kgkasecsqy qqilhtnlvy |
| 61 | latiadsnqn mqsllpappt qtmpmgpggm sqsgpppppr shnmpsdgmv gggppaphmq |
| 121 | nqmngqmpgp mmhqqppsqq ynmppggagh yqgqqapmgl mgqvnqgshm mgqrqmppyr |
| 181 | ppqqgppqqy sgqedyygdq yshggqgppe gmnqqyypdg nsqygqqqda yqgpppqqgy |
| 241 | ppqqqqypgq qgypgqqqsy gpsqggpgpq ypnypqgqgq qyggyrptqp gppqppqqrp |
| 301 | ygydqgqygn yqq |

SEQ ID NO: 45 Mouse SS18 cDNA Sequence variant 4 (NM_001161371.1; CDS:
180-1121)

| | |
|---|---|
| 1 | ccttgctggg agctgcggct cagcgttaag gccaagccgg ccagcgaggg acgcggcccg |
| 61 | ggagcatcct ccccccaccg cgcgccctaa ggtggaactg cccggaggcg ggcgtcgggc |
| 121 | ccccagctcc gcgggccctg gagcgctcgg gactcgctga tcgcgggctc ggcggcaaca |
| 181 | tgtctgtggc gttcgcagcc ccgaggcagc ggggcaaggg cgaaatcacg cccgccgcca |
| 241 | tccagaagat gctggatgaa aacaaccatc ttattcagtg tataatggac tatcagaaca |
| 301 | aagggaaggc ctcggagtgc tcgcagtatc agcagatatt gcatacaaac ctggtatacc |
| 361 | ttgctacaat agcagactct aatcaaaata tgcagtctct cttaccagca ccgcccacac |
| 421 | agactatgcc aatgggtcct ggagggatga gtcagagtgg ccctccaccc cctccccgct |
| 481 | ctcacaacat gccttcagat ggaatggtgg gtgggggccc tcctgcacca cacatgcaga |
| 541 | accagatgaa cggccagatg cctgggccga tgatgcacca gcagcctcct tctcagcagt |
| 601 | acaatatgcc acctggaggg gcacagcatt accaaggaca gcaggcgccc atggggctga |
| 661 | tgggccaagt taaccaaggc agtcacatga tgggccagcg acagatgcct ccctacagac |
| 721 | ctccgcaaca gggcccacca cagcagtact caggccagga agactattat ggggaccaat |
| 781 | acagtcatgg tggacaaggt cctccagaag gcatgaacca gcaatattac cctgatggaa |
| 841 | actcccagta tggccaacag caagacgctt accagggacc acctccacag caaggatacc |
| 901 | caccccagca gcagcagtac ccgggacagc agggataccc agggcagcag cagagctatg |
| 961 | gtccttcgca gggcggtcca ggtcctcagt atcctaatta tcctcagggt caaggtcagc |
| 1021 | agtatggggg ctatagacca acacagccag gaccacccca gccaccccag cagaggcctt |
| 1081 | atgggtacga ccagggacag tatggaaatt accagcagtg aaaatgtcct tacattccaa |
| 1141 | tagccagtac ctattagcag gcacgttgtc acagcactgc accatggaca cccccctggg |
| 1201 | aagactcctt ccattccagc taggtttttg ggaaaacctt tggctaagtg gctgcttcgt |
| 1261 | cagcaagtag ctgttatggt ttagtttgta aaggcttcgt agctaccgat gcacctgatt |
| 1321 | tcacgtttct actctagatg gcaacattgg acagaaaatg cattgacgtg aggagtttgc |
| 1381 | agcggtttca gaactgtgct gcaaatggac tgtcacagcc tgaaaggtgt gagcagctgg |
| 1441 | gtgtgtgttc gcggagcttc agggggtttc atacttttcc accgattatt ttgtaagggg |
| 1501 | aagggggaaa tgtacacttt ttacagcagc aatattttgt ctattatgtt tatttcatgt |
| 1561 | gataaatatg caaagcggta cactacacac tgggcagaat cagaacccct gttaatgtgg |
| 1621 | agtgtggtag atgctcggtg ctgtggtgct ctgaagacag gcgaggggag gcagaagccc |
| 1681 | accacaggcc cgctgttagt tcttagagga aactcctctc tctcttatct accagattag |
| 1741 | caagggcgct gtgatacagt tttttgagta caaagacatt ttttaaaaag ccttccagtt |
| 1801 | ttgtgcatta aaaccttttt gtaaatatgg tttataatac tgttttcaaa cgcaaggcaa |
| 1861 | taattatgtt gcatctgtga actttggcag gtttgtgtaa aaggagggaa gcctctctta |
| 1921 | aaacagcaat aacagaaaag gaggaagcgg gatgttttta ccttgtcttg taatcaggga |
| 1981 | gctctcacca cgtcagagag gaggcagcat tggtctcacc ttactgtttt ttacattacc |
| 2041 | atgattggtt catggagcag ggaggagtcc acgagacttc acacgcttgt gctttaactt |
| 2101 | tcttaactgg gcacaagcaa agggcgcctt cgtgttcctc tcttcatctt agttaatgcg |
| 2161 | cgaggaaaat gctttgatgg ccatttctca ttcgcactga aagccgagag gtgacatttt |
| 2221 | acggtttctt gtttttaagc acgacatact taatcattat ttgagactga ttattttagc |
| 2281 | taaatttggg gatatgccat ggggcaagaa aacatgtact gagagatttc taaacacatc |
| 2341 | tatttaagca tactttaaaa atatctagcc caaaggtaag ttgctgtatc ctcacagttg |
| 2401 | tctgcatcca gggaatatga ctgaatataa catatctttg taattgaatt agtttttgcc |
| 2461 | acttctaact gaaaacagaa cagaaggagt gccataaatg caaagaagca aagtgtactg |
| 2521 | ttgtcaacat actgtaatca gaggaggggt ttcaatgtgt ctggatgaga gtgtgtgtgt |
| 2581 | ttaaggtcag agtatagggt gttcttcaac ttggacagta gaaaataggc atcaagtgtg |
| 2641 | aaccggtgag gcgtggacag ccttcttgtg actgagatgc ttgtaagttc tgtgccaggt |
| 2701 | tctccaccac tgtgtacttt attgctattt aaaactgtat caactctaac gaaagaataa |
| 2761 | attatttgtg attttaaaaa aaaaaaaaaa aaaaa |

SEQ ID NO: 46 Human ARID1A cDNA Sequence Variant 1 (NM_006015.4, CDS:
from 374 to 7231)

| | |
|---|---|
| 1 | cagaaagcgg agagtcacag cggggccagg ccctggggag cggagcctcc accgcccccc |
| 61 | tcattcccag gcaagggctt ggggggaatg agccgggaga gccgggtccc gagcctacag |
| 121 | agccgggagc agctgagccg ccggcgcctc ggccgccgcc gccgcctcct cctcctccgc |
| 181 | cgccgccagc ccggagcctg agccggcggg gcggggggga gaggagcgag cgcagcgcag |
| 241 | cagcggagcc ccgcgaggcc cgcccgggcg ggtggggagg gcagcccggg ggactgggcc |
| 301 | ccggggcggg gtgggagggg gggagaagac gaagacaggg ccgggtctct ccgcggacga |
| 361 | gacagcgggg atcatggccg cgcaggtcgc ccccgccgcc gccagcagcc tgggcaaccc |
| 421 | gccgccgccg ccgccctcgg agctgaagaa agccgagcag cagcagcggg aggaggcggg |
| 481 | gggcgaggcg gcggcggcgg cagcggccga gcgcggggaa atgaaggcag ccgccgggca |
| 541 | ggaaagcgag ggccccgccg tggggccgcc gcagccgctg ggaaaggagc tgcaggacgg |
| 601 | ggccgagagc aatgggggtg gcggcggcgg cggagccggc agcggcggcg ggcccggcgc |
| 661 | ggagccggac ctgaagaact cgaacgggaa cgcgggccct aggcccgccc tgaacaataa |
| 721 | cctcacggag ccgcccggcg gcggcggtgg cggcagcagc gatggggtgg gggcgcctcc |
| 781 | tcactcagcc gcggccgcct tgccgccccc agcctacggc ttcgggcaac cctacggccg |
| 841 | gagcccgtct gccgtcgccg ccgccgcggc cgccgtcttc caccaacaac atggcggaca |
| 901 | acaaagccct ggcctggcag cgctgcagag cggcggcggc gggggcctgg agccctacgc |
| 961 | ggggccccag cagaactctc acgaccacgg cttccccaac caccagtaca actcctacta |
| 1021 | ccccaaccgc agcgcctacc ccccgcccgc cccggcctac gcgctgagct ccccgagagg |
| 1081 | tggcactccg ggctccggcg cggcggcggc tgccggctcc aagccgcctc cctcctccag |
| 1141 | cgcctccgcc tcctcgtcgt cttcgtcctt cgctcagcag cgcttcgggg ccatgggggg |
| 1201 | aggcggcccc tccgcggccg gcgggggaac tccccagccc accgccaccc ccaccctcaa |
| 1261 | ccaactgctc acgtcgccca gctcggcccg gggctaccag ggctaccccg ggggcgacta |
| 1321 | cagtggcggg ccccaggacg ggggcgccgg caagggcccg gcggacatgg cctcgcagtg |
| 1381 | ttggggggct gcggcggcgg cagctgcggc ggcggccgcc tcgggagggg cccaacaaag |
| 1441 | gagccaccac gcgcccatga gccccgggag cagcggcggc ggggggcagc cgctcgcccg |
| 1501 | gacccctcag ccatccagtc caatggatca gatgggcaag atgagacctc agccatatgg |
| 1561 | cgggactaac ccatactcgc agcaacaggg acctccgtca ggaccgcagc aaggacatgg |
| 1621 | gtacccaggg cagccatacg ggtcccagac cccgcagcgg tacccgatga ccatgcaggg |
| 1681 | ccgggcgcag agtgccatgg gcggcctctc ttatacacag cagattcctc cttatggaca |
| 1741 | acaaggcccc agcgggtatg gtcaacaggg ccagactcca tattacaacc agcaaagtcc |
| 1801 | tcaccctcag cagcagcagc caccctactc ccagcaacca ccgtcccaga cccctcatgc |
| 1861 | ccaaccttcg tatcagcagc agccacagtc tcaaccacca cagctccagt cctctcagcc |
| 1921 | tccatactcc cagcagccat cccagcctcc acatcagcag tccccggctc catacccctc |
| 1981 | ccagcagtcg acgacacagc agcaccccca gagccagccc ccctactcac agccacaggc |
| 2041 | tcagtctcct taccagcagc agcaacctca gcagccagca ccctcgacgc tctcccagca |
| 2101 | ggctgcgtat cctcagcccc agtctcagca gtcccagcaa actgcctatt cccagcagcg |
| 2161 | cttccctcca ccgcaggagc tatctcaaga ttcatttggg tctcaggcat cctcagcccc |
| 2221 | ctcaatgacc tccagtaagg gagggcaaga agatatgaac ctgagccttc agtcaagacc |
| 2281 | ctccagcttg cctgatctat ctggttcaat agatgacctc cccatgggga cagaaggagc |
| 2341 | tctgagtcct ggagtgagca catcagggat ttccagcagc caaggagagc agagtaatcc |
| 2401 | agctcagtct cctttctctc ctcatacctc ccctcacctg cctggcatcc gaggcccttc |
| 2461 | cccgtcccct gttggctctc ccgccagtgt tgctcagtct cgctcaggac cactctcgcc |
| 2521 | tgctgcagtg ccaggcaacc agatgccacc tcggccaccc agtggccagt cggacagcat |
| 2581 | catgcatcct tccatgaacc aatcaagcat tgcccaagat cgaggttata tgcagaggaa |
| 2641 | cccccagatg ccccagtaca gttcccccca gcccggctca gccttatctc cgcgtcagcc |
| 2701 | ttccggagga cagatacaca caggcatggg ctcctaccag cagaactcca tggggagcta |
| 2761 | tggtccccag gggggtcagt atggcccaca aggtggctac cccaggcagc caaactataa |
| 2821 | tgccttgccc aatgccaact accccagtgc aggcatggct ggaggcataa accccatggg |
| 2881 | tgccggaggt caaatgcatg gacagcctgg catcccacct tatggcacac tccctccagg |
| 2941 | gaggatgagt cacgcctcca tgggcaaccg gccttatggc cctaacatgg ccaatatgcc |
| 3001 | acctcaggtt gggtcaggga tgtgtccccc accagggggc atgaaccgga aaacccaaga |
| 3061 | aactgctgtc gccatgcatg ttgctgccaa ctctatccaa aacaggccgc caggctaccc |
| 3121 | caatatgaat caagggggca tgatgggaac tggacctcct tatggacaag ggattaatag |
| 3181 | tatggctggc atgatcaacc ctcagggacc cccatattcc atgggtggaa ccatggccaa |
| 3241 | caattctgca gggatggcag ccagcccaga gatgatgggc cttggggatg taaagttaac |
| 3301 | tccagccacc aaaatgaaca acaaggcaga tgggacaccc aagacagaat ccaaatccaa |
| 3361 | gaaatccagt tcttctacta caaccaatga gaagatcacc aagttgtatg agctgggtgg |
| 3421 | tgagcctgag aggaagatgt gggtggaccg ttatctggcc ttcactgagg agaaggccat |
| 3481 | gggcatgaca aatctgcctg ctgtgggtag gaaacctctg gacctctatc gcctctatgt |
| 3541 | gtctgtgaag gagattggtg gattgactca ggtcaacaag aacaaaaaat ggcgggaact |
| 3601 | tgcaaccaac ctcaatgtgg gcacatcaag cagtgctgcc agctccttga aaaagcagta |
| 3661 | tatccagtgt ctctatgcct ttgaatgcaa gattgaacgg ggagaagacc ctcccccaga |
| 3721 | catctttgca gctgctgatt ccaagaagtc ccagcccaag atccagcctc cctctcctgc |
| 3781 | gggatcagga tctatgcagg ggccccagac tccccagtca accagcagtt ccatggcaga |
| 3841 | aggaggagac ttaaagccac caactccagc atccacacca cacagtcaga tccccccatt |
| 3901 | gccaggcatg agcaggagca attcagttgg gatccaggat gcctttaatg atggaagtga |
| 3961 | ctccacattc cagaagcgga attccatgac tccaaaccct gggtatcagc ccagtatgaa |
| 4021 | tacctctgac atgatggggc gcatgtccta tgagccaaat aaggatcctt atggcagcat |
| 4081 | gaggaaagct ccagggagtg atcccttcat gtcctcaggg cagggcccca acggcgggat |
| 4141 | gggtgacccc tacagtcgtg ctgccggccc tgggctagga aatgtggcga tgggaccacg |
| 4201 | acagcactat ccctatggag gtccttatga cagagtgagg acggagcctg gaatagggcc |
| 4261 | tgagggaaac atgagcactg gggccccaca gccgaatctc atgccttcca acccagactc |
| 4321 | ggggatgtat tctcctagcc gctacccccc gcagcagcag cagcagcagc agcaacgaca |
| 4381 | tgattcctat ggcaatcagt tctccaccca aggcacccct tctggcagcc ccttccccag |
| 4441 | ccagcagact acaatgtatc aacagcaaca gcagaattac aagcggccaa tggatggcac |
| 4501 | atatggccct cctgccaagc ggcacgaagg ggagatgtac agcgtgccat acagcactgg |
| 4561 | gcaggggcag cctcagcagc agcagttgcc cccagcccag ccccagcctg ccagccagca |
| 4621 | acaagctgcc cagccttccc ctcagcaaga tgtatacaac cagtatggca atgcctatcc |
| 4681 | tgccactgcc acagctgcta ctgagcgccg accagcaggc ggcccccaga accaatttcc |
| 4741 | attccagttt ggccgagacc gtgtctctgc accccctggc accaatgccc agcaaaacat |
| 4801 | gccaccacaa atgatgggcg gccccataca ggcatcagct gaggttgctc agcaaggcac |
| 4861 | catgtggcag gggcgtaatg acatgaccta taattatgcc aacaggcaga gcacgggctc |
| 4921 | tgccccccag ggccccgcct atcatggcgt gaaccgaaca gatgaaatgc tgcacacaga |
| 4981 | tcagagggcc aaccacgaag gctcgtggcc ttcccatggc acacgccagc ccccatatgg |
| 5041 | tccctctgcc cctgtgcccc ccatgacaag gccccctcca tctaactacc agcccccacc |
| 5101 | aagcatgcag aatcacattc ctcaggtatc cagccctgct cccctgcccc ggccaatgga |
| 5161 | gaaccgcacc tctcctagca agtctccatt cctgcactct gggatgaaaa tgcagaaggc |
| 5221 | aggtccccca gtacctgcct cgcacatagc acctgcccct gtgcagcccc ccatgattcg |
| 5281 | gcgggatatc accttcccac ctggctctgt tgaagccaca cagcctgtgt tgaagcagag |
| 5341 | gaggcggctc acaatgaaag acattggaac cccggaggca tggcgggtaa tgatgtccct |
| 5401 | caagtctggt ctcctggcag agagcacatg ggcattagat accatcaaca tcctgctgta |
| 5461 | tgatgacaac agcatcatga ccttcaacct cagtcagctc ccagggttgc tagagctcct |
| 5521 | tgtagaatat ttccgacgat gcctgattga gatctttggc attttaaagg agtatgaggt |
| 5581 | gggtgaccca ggacagagaa cgctactgga tcctgggagg ttcagcaagg tgtctagtcc |
| 5641 | agctcccatg gagggtgggg aagaagaaga agaacttcta ggtcctaaac tagaagagga |
| 5701 | agaagaagag gaagtagttg aaaatgatga ggagatagcc ttttcaggca aggacaagcc |
| 5761 | agcttcagag aatagtgagg agaagctgat cagtaagttt gacaagcttc cagtaaagat |
| 5821 | cgtacagaag aatgatccat ttgtggtgga ctgctcagat aagcttgggc gtgtgcagga |
| 5881 | gtttgacagt ggcctgctgc actggcggat tggtgggggg gacaccactg agcatatcca |
| 5941 | gacccacttc gagagcaaga cagagctgct gccttcccgg cctcacgcac cctgcccacc |
| 6001 | agcccctcgg aagcatgtga caacagcaga gggtacacca gggacaacag accaggaggg |
| 6061 | gcccccacct gatggacctc cagaaaaacg gatcacagcc actatggatg acatgttgtc |
| 6121 | tactcggtct agcaccttga ccgaggatgg agctaagagt tcagaggcca tcaaggagag |
| 6181 | cagcaagttt ccatttggca ttagcccagc acagagccac cggaacatca agatcctaga |
| 6241 | ggacgaaccc cacagtaagg atgagacccc actgtgtacc cttctggact ggcaggattc |
| 6301 | tcttgccaag cgctgcgtct gtgtgtccaa taccattcga agcctgtcat ttgtgccagg |
| 6361 | caatgacttt gagatgtcca aacacccagg gctgctgctc atcctgggca agctgatcct |
| 6421 | gctgcaccac aagcacccag aacggaagca ggcaccacta acttatgaaa aggaggagga |
| 6481 | acaggaccaa ggggtgagct gcaacaaagt ggagtggtgg tgggactgct tggagatgct |
| 6541 | ccgggaaaac accttggtta cactcgccaa catctcgggg cagttggacc tatctccata |
| 6601 | ccccgagagc atttgcctgc ctgtcctgga cggactccta cactgggcag tttgcccttc |
| 6661 | agctgaagcc caggacccct tttccaccct gggccccaat gccgtccttt ccccgcagag |
| 6721 | actggtcttg gaaaccctca gcaaactcag catccaggac aacaatgtgg acctgattct |
| 6781 | ggccacaccc cccttcagcc gcctggagaa gttgtatagc actatggtgc gcttcctcag |
| 6841 | tgaccgaaag aacccggtgt gccgggagat ggctgtggta ctgctggcca acctggctca |
| 6901 | gggggacagc ctggcagctc gtgccattgc agtgcagaag ggcagtatcg gcaacctcct |
| 6961 | gggcttccta gaggacagcc ttgccgccac acagttccag cagagccagg ccagcctcct |
| 7021 | ccacatgcag aacccaccct ttgagccaac tagtgtggac atgatgcggc gggctgcccg |
| 7081 | cgcgctgctt gccttggcca aggtggacga gaaccactca gagtttactc tgtacgaatc |
| 7141 | acggctgttg gacatctcgg tatcaccgtt gatgaactca ttggtttcac aagtcatttg |
| 7201 | tgatgtactg tttttgattg gccagtcatg acagccgtgg gacacctccc ccccccgtgt |
| 7261 | gtgtgtgcgt gtgtggagaa cttagaaact gactgttgcc ctttatttat gcaaaaccac |
| 7321 | ctcagaatcc agtttaccct gtgctgtcca gcttctccct tgggaaaaag tctctcctgt |
| 7381 | ttctctctcc tccttccacc tcccctccct ccatcacctc acgcctttct gttccttgtc |
| 7441 | ctcaccttac tcccctcagg accctacccc accctctttg aaaagacaaa gctctgccta |
| 7501 | catagaagac tttttttatt ttaaccaaag ttactgttgt ttacagtgag tttggggaaa |
| 7561 | aaaaataaaa taaaaatggc tttcccagtc cttgcatcaa cgggatgcca catttcataa |
| 7621 | ctgtttttaa tggtaaaaaa aaaaaaaaaa aatacaaaaa aaaattctga aggacaaaaa |
| 7681 | aggtgactgc tgaactgtgt gtggtttatt gttgtacatt cacaatcttg caggagccaa |
| 7741 | gaagttcgca gttgtgaaca gaccctgttc actggagagg cctgtgcagt agagtgtaga |
| 7801 | ccctttcatg tactgtactg tacacctgat actgtaaaca tactgtaata ataatgtctc |
| 7861 | acatggaaac agaaaacgct gggtcagcag caagctgtag tttttaaaaa tgtttttagt |
| 7921 | taaacgttga ggagaaaaaa aaaaaaggct tttcccccaa agtatcatgt gtgaacctac |
| 7981 | aacaccctga cctctttctc tcctccttga ttgtatgaat aaccctgaga tcacctctta |
| 8041 | gaactggttt taacctttag ctgcagcggc tacgctgcca cgtgtgtata tatatgacgt |
| 8101 | tgtacattgc acataccctt ggatccccac agtttggtcc tcctcccagc taccccttta |
| 8161 | tagtatgacg agttaacaag ttggtgacct gcacaaagcg agacacagct atttaatctc |
| 8221 | ttgccagata tcgcccctct tggtgcgatg ctgtacaggt ctctgtaaaa agtccttgct |
| 8281 | gtctcagcag ccaatcaact tatagtttat ttttttctgg gtttttgttt tgttttgttt |
| 8341 | tctttctaat cgaggtgtga aaaagttcta ggttcagttg aagttctgat gaagaaacac |
| 8401 | aattgagatt ttttcagtga taaaatctgc atatttgtat ttcaacaatg tagctaaaac |
| 8461 | ttgatgtaaa ttcctccttt ttttcctttt ttggcttaat gaatatcatt tattcagtat |
| 8521 | gaaatcttta tactatatgt tccacgtgtt aagaataaat gtacattaaa tcttggtaag |
| 8581 | acttt |

SEQ ID NO: 47 Human ARID1A Amino Acid Sequence isoform A (NP_006006.3)

| | |
|---|---|
| 1 | maaqvapaaa sslgnppppp pselkkaeqq qreeaggeaa aaaaaergem kaaagqeseg |
| 61 | pavgppqplg kelqdgaesn gggggggags gggpgaepdl knsngnagpr palnnnltep |
| 121 | pggggggssd gvgapphsaa aalpppaygf gqpygrspsa vaaaaaavfh qqhggqqspg |
| 181 | laalqsgggg glepyagpqq nshdhgfpnh qynsyypnrs aypppapaya lssprggtpg |
| 241 | sgaaaaagsk pppsssasas sssssfaqqr fgamggggps aagggtpqpt atptlnqllt |
| 301 | spssargyqg ypggdysggp qdggagkgpa dmasqcwgaa aaaaaaaaas ggaqqrshha |
| 361 | pmspgssggg gqplartpqp sspmdqmgkm rpqpyggtnp ysqqqgppsg pqqghgypgq |
| 421 | pygsqtpqry pmtmqgraqs amgglsytqq ippygqqgps gygqqgqtpy ynqqsphpqq |
| 481 | qqppysqqpp sqtphaqpsy qqqpqsqppq lqssqppysq qpsqpphqqs papypsqqst |
| 541 | tqqhpqsqpp ysqpqaqspy qqqqpqqpap stlsqqaayp qpqsqqsqqt aysqqrfppp |
| 601 | qelsqdsfgs qassapsmts skggqedmnl slqsrpsslp dlsgsiddlp mgtegalspg |
| 661 | vstsgisssq geqsnpaqsp fsphtsphlp girgpspspv gspasvaqsr sgplspaavp |
| 721 | gnqmpprpps gqsdsimhps mnqssiaqdr gymqrnpqmp qysspqpgsa lsprqpsggq |
| 781 | ihtgmgsyqq nsmgsygpqg gqygpqggyp rqpnynalpn anypsagmag ginpmgaggq |
| 841 | mhgqpgippy gtlppgrmsh asmgnrpygp nmanmppqvg sgmcpppggm nrktqetava |
| 901 | mhvaansiqn rppgypnmnq ggmmgtgppy gqginsmagm inpqgppysm ggtmannsag |
| 961 | maaspemmgl gdvkltpatk mnnkadgtpk teskskksss stttnekitk lyelggeper |
| 1021 | kmwvdrylaf teekamgmtn lpavgrkpld lyrlyvsvke iggltqvnkn kkwrelatnl |
| 1081 | nvgtsssaas slkkqyiqcl yafeckierg edpppdifaa adskksqpki qppspagsgs |
| 1141 | mqgpqtpqst sssmaeggdl kpptpastph sqipplpgms rsnsvgiqda fndgsdstfq |
| 1201 | krnsmtpnpg yqpsmntsdm mgrmsyepnk dpygsmrkap gsdpfmssgq gpnggmgdpy |
| 1261 | sraagpglgn vamgprqhyp yggpydrvrt epgigpegnm stgapqpnlm psnpdsgmys |
| 1321 | psryppqqqq qqqqrhdsyg nqfstqgtps gspfpsqqtt myqqqqqnyk rpmdgtygpp |
| 1381 | akrhegemys vpystgqgqp qqqqlppaqp qpasqqqaaq pspqqdvynq ygnaypatat |
| 1441 | aaterrpagg pqnqfpfqfg rdrvsappgt naqqnmppqm mggpiqasae vaqqgtmwqg |
| 1501 | rndmtynyan rqstgsapqg payhgvnrtd emlhtdqran hegswpshgt rqppygpsap |
| 1561 | vppmtrppps nyqpppsman hipqvsspap lprpmenrts pskspflhsg mkmqkagppv |
| 1621 | pashiapapv qppmirrdit fppgsveatq pvlkqrrrlt mkdigtpeaw rvmmslksgl |
| 1681 | laestwaldt inillyddns imtfnlsqlp gllellveyf rrclieifgi lkeyevgdpg |
| 1741 | qrtlldpgrf skvsspapme ggeeeeellg pkleeeeeee vvendeeiaf sgkdkpasen |
| 1801 | seekliskfd klpvkivqkn dpfvvdcsdk lgrvqefdsg llhwrigggd ttehiqthfe |
| 1861 | sktellpsrp hapcppaprk hvttaegtpg ttdqegpppd gppekritat mddmlstrss |
| 1921 | tltedgakss eaikesskfp fgispaqshr nikiledeph skdetplctl ldwqdslakr |
| 1981 | cvcvsntirs lsfvpgndfe mskhpgllli lgklillhhk hperkqaplt yekeeeqdqg |
| 2041 | vscnkvewww dclemlrent lvtlanisgq ldlspypesi clpvldgllh wavcpsaeaq |
| 2101 | dpfstlgpna vlspqrlvle tlsklsiqdn nvdlilatpp fsrleklyst mvrflsdrkn |
| 2161 | pvcremavvl lanlaqgdsl aaraiavqkg signllgfle dslaatqfqq sqasllhmqn |
| 2221 | ppfeptsvdm mrraaralla lakvdenhse ftlyesrlld isvsplmnsl vsqvicdvlf |
| 2281 | ligqs |

SEQ ID NO: 48 Human ARID1A cDNA Sequence Variant 2 (NM_139135.2, CDS:
from 374 to 6580)

| | |
|---|---|
| 1 | cagaaagcgg agagtcacag cggggccagg ccctggggag cggagcctcc accgcccccc |
| 61 | tcattcccag gcaagggctt ggggggaatg agccgggaga gccgggtccc gagcctacag |
| 121 | agccgggagc agctgagccg ccggcgcctc ggccgccgcc gccgcctcct cctcctccgc |
| 181 | cgccgccagc ccggagcctg agccggcggg gcggggggga gaggagcgag cgcagcgcag |
| 241 | cagcggagcc ccgcgaggcc cgcccgggcg ggtggggagg gcagcccggg ggactgggcc |
| 301 | ccggggcggg gtgggagggg gggagaagac gaagacaggg ccgggtctct ccgcggacga |
| 361 | gacagcgggg atcatggccg cgcaggtcgc ccccgccgcc gccagcagcc tgggcaaccc |
| 481 | gggcgaggcg gcggcggcgg cagcggccga gcgcggggaa atgaaggcag ccgccgggca |
| 541 | ggaaagcgag ggccccgccg tggggccgcc gcagccgctg ggaaaggagc tgcaggacgg |
| 601 | ggccgagagc aatgggggtg gcggcggcgg cggagccggc agcggcggcg ggcccggcgc |
| 661 | ggagccggac ctgaagaact cgaacgggaa cgcgggccct aggcccgccc tgaacaataa |
| 721 | cctcacggag ccgcccggcg gcggcggtgg cggcagcagc gatggggtgg gggcgcctcc |
| 781 | tcactcagcc gcggccgcct tgccgccccc agcctacggc ttcgggcaac cctacggccg |
| 841 | gagcccgtct gccgtcgccg ccgccgcggc cgccgtcttc caccaacaac atggcggaca |
| 901 | acaaagccct ggcctggcag cgctgcagag cggcggcggc gggggcctgg agccctacgc |
| 961 | ggggccccag cagaactctc acgaccacgg cttccccaac caccagtaca actcctacta |
| 1021 | ccccaaccgc agcgcctacc ccccgcccgc cccggcctac gcgctgagct ccccgagagg |
| 1081 | tggcactccg ggctccggcg cggcggcggc tgccggctcc aagccgcctc cctcctccag |
| 1141 | cgcctccgcc tcctcgtcgt cttcgtcctt cgctcagcag cgcttcgggg ccatgggggg |
| 1201 | aggcggcccc tccgcggccg gcgggggaac tccccagccc accgccaccc ccaccctcaa |
| 1261 | ccaactgctc acgtcgccca gctcggcccg gggctaccag ggctaccccg ggggcgacta |
| 1321 | cagtggcggg ccccaggacg ggggcgccgg caagggcccg gcggacatgg cctcgcagtg |
| 1381 | ttggggggct gcggcggcgg cagctgcggc ggcggccgcc tcgggagggg cccaacaaag |
| 1441 | gagccaccac gcgcccatga gccccgggag cagcggcggc ggggggcagc cgctcgcccg |
| 1501 | gacccctcag ccatccagtc caatggatca gatgggcaag atgagacctc agccatatgg |
| 1561 | cgggactaac ccatactcgc agcaacaggg acctccgtca ggaccgcagc aaggacatgg |
| 1621 | gtacccaggg cagccatacg ggtcccagac cccgcagcgg tacccgatga ccatgcaggg |
| 1681 | ccgggcgcag agtgccatgg gcggcctctc ttatacacag cagattcctc cttatggaca |
| 1741 | acaaggcccc agcgggtatg gtcaacaggg ccagactcca tattacaacc agcaaagtcc |
| 1801 | tcaccctcag cagcagcagc caccctactc ccagcaacca ccgtcccaga cccctcatgc |
| 1861 | ccaaccttcg tatcagcagc agccacagtc tcaaccacca cagctccagt cctctcagcc |
| 1921 | tccatactcc cagcagccat cccagcctcc acatcagcag tccccggctc catacccctc |
| 1981 | ccagcagtcg acgacacagc agcaccccca gagccagccc ccctactcac agccacaggc |
| 2041 | tcagtctcct taccagcagc agcaacctca gcagccagca ccctcgacgc tctcccagca |
| 2101 | ggctgcgtat cctcagcccc agtctcagca gtcccagcaa actgcctatt cccagcagcg |
| 2161 | cttccctcca ccgcaggagc tatctcaaga ttcatttggg tctcaggcat cctcagcccc |
| 2221 | ctcaatgacc tccagtaagg gagggcaaga agatatgaac ctgagccttc agtcaagacc |
| 2281 | ctccagcttg cctgatctat ctggttcaat agatgacctc cccatgggga cagaaggagc |
| 2341 | tctgagtcct ggagtgagca catcagggat ttccagcagc caaggagagc agagtaatcc |
| 2401 | agctcagtct cctttctctc ctcatacctc ccctcacctg cctggcatcc gaggcccttc |
| 2461 | cccgtcccct gttggctctc ccgccagtgt tgctcagtct cgctcaggac cactctcgcc |
| 2521 | tgctgcagtg ccaggcaacc agatgccacc tcggccaccc agtggccagt cggacagcat |
| 2581 | catgcatcct tccatgaacc aatcaagcat tgcccaagat cgaggttata tgcagaggaa |
| 2641 | cccccagatg ccccagtaca gttcccccca gcccggctca gccttatctc cgcgtcagcc |
| 2701 | ttccggagga cagatacaca caggcatggg ctcctaccag cagaactcca tggggagcta |
| 2761 | tggtccccag gggggtcagt atggcccaca aggtggctac cccaggcagc caaactataa |
| 2821 | tgccttgccc aatgccaact accccagtgc aggcatggct ggaggcataa accccatggg |
| 2881 | tgccggaggt caaatgcatg gacagcctgg catcccacct tatggcacac tccctccagg |
| 2941 | gaggatgagt cacgcctcca tgggcaaccg gccttatggc cctaacatgg ccaatatgcc |
| 3001 | acctcaggtt gggtcaggga tgtgtccccc accagggggc atgaaccgga aaacccaaga |
| 3061 | aactgctgtc gccatgcatg ttgctgccaa ctctatccaa aacaggccgc caggctaccc |
| 3121 | caatatgaat caagggggca tgatgggaac tggacctcct tatggacaag ggattaatag |
| 3181 | tatggctggc atgatcaacc ctcagggacc cccatattcc atgggtggaa ccatggccaa |
| 3241 | caattctgca gggatggcag ccagcccaga gatgatgggc cttggggatg taaagttaac |
| 3301 | tccagccacc aaaatgaaca acaaggcaga tgggacaccc aagacagaat ccaaatccaa |
| 3361 | gaaatccagt tcttctacta caaccaatga gaagatcacc aagttgtatg agctgggtgg |
| 3421 | tgagcctgag aggaagatgt gggtggaccg ttatctggcc ttcactgagg agaaggccat |
| 3481 | gggcatgaca aatctgcctg ctgtgggtag gaaacctctg gacctctatc gcctctatgt |
| 3541 | gtctgtgaag gagattggtg gattgactca ggtcaacaag aacaaaaaat ggcgggaact |
| 3601 | tgcaaccaac ctcaatgtgg gcacatcaag cagtgctgcc agctccttga aaaagcagta |
| 3661 | tatccagtgt ctctatgcct ttgaatgcaa gattgaacgg ggagaagacc ctcccccaga |
| 3721 | catctttgca gctgctgatt ccaagaagtc ccagcccaag atccagcctc cctctcctgc |
| 3781 | gggatcagga tctatgcagg ggccccagac tccccagtca accagcagtt ccatggcaga |
| 3841 | aggaggagac ttaaagccac caactccagc atccacacca cacagtcaga tccccccatt |
| 3901 | gccaggcatg agcaggagca attcagttgg gatccaggat gcctttaatg atggaagtga |
| 3961 | ctccacattc cagaagcgga attccatgac tccaaaccct gggtatcagc ccagtatgaa |
| 4021 | tacctctgac atgatggggc gcatgtccta tgagccaaat aaggatcctt atggcagcat |
| 4081 | gaggaaagct ccagggagtg atcccttcat gtcctcaggg cagggcccca acggcgggat |
| 4141 | gggtgacccc tacagtcgtg ctgccggccc tgggctagga aatgtggcga tgggaccacg |
| 4201 | acagcactat ccctatggag gtccttatga cagagtgagg acggagcctg gaatagggcc |
| 4261 | tgagggaaac atgagcactg gggccccaca gccgaatctc atgccttcca acccagactc |
| 4321 | ggggatgtat tctcctagcc gctacccccc gcagcagcag cagcagcagc agcaacgaca |
| 4381 | tgattcctat ggcaatcagt tctccaccca aggcacccct tctggcagcc ccttccccag |
| 4441 | ccagcagact acaatgtatc aacagcaaca gcaggtatcc agccctgctc ccctgccccg |
| 4501 | gccaatggag aaccgcacct ctcctagcaa gtctccattc ctgcactctg ggatgaaaat |
| 4561 | gcagaaggca ggtcccccag tacctgcctc gcacatagca cctgcccctg tgcagccccc |
| 4621 | catgattcgg cgggatatca ccttcccacc tggctctgtt gaagccacac agcctgtgtt |
| 4681 | gaagcagagg aggcggctca caatgaaaga cattggaacc ccggaggcat ggcgggtaat |
| 4741 | gatgtccctc aagtctggtc tcctggcaga gagcacatgg gcattagata ccatcaacat |
| 4801 | cctgctgtat gatgacaaca gcatcatgac cttcaacctc agtcagctcc cagggttgct |
| 4861 | agagctcctt gtagaatatt tccgacgatg cctgattgag atctttggca ttttaaagga |
| 4921 | gtatgaggtg ggtgacccag gacagagaac gctactggat cctgggaggt tcagcaaggt |
| 4981 | gtctagtcca gctcccatgg agggtgggga agaagaagaa gaacttctag gtcctaaact |
| 5041 | agaagaggaa gaagaagagg aagtagttga aaatgatgag gagatagcct tttcaggcaa |
| 5101 | ggacaagcca gcttcagaga atagtgagga gaagctgatc agtaagtttg acaagcttcc |
| 5161 | agtaaagatc gtacagaaga atgatccatt tgtggtggac tgctcagata agcttgggcg |
| 5221 | tgtgcaggag tttgacagtg gcctgctgca ctggcggatt ggtggggggg acaccactga |
| 5281 | gcatatccag acccacttcg agagcaagac agagctgctg ccttcccggc ctcacgcacc |
| 5341 | ctgcccacca gcccctcgga agcatgtgac aacagcagag ggtacaccag ggacaacaga |
| 5401 | ccaggagggg cccccacctg atggacctcc agaaaaacgg atcacagcca ctatggatga |
| 5461 | catgttgtct actcggtcta gcaccttgac cgaggatgga gctaagagtt cagaggccat |
| 5521 | caaggagagc agcaagtttc catttggcat tagcccagca cagagccacc ggaacatcaa |
| 5581 | gatcctagag gacgaacccc acagtaagga tgagacccca ctgtgtaccc ttctggactg |
| 5641 | gcaggattct cttgccaagc gctgcgtctg tgtgtccaat accattcgaa gcctgtcatt |
| 5701 | tgtgccaggc aatgactttg agatgtccaa acacccaggg ctgctgctca tcctgggcaa |
| 5761 | gctgatcctg ctgcaccaca agcacccaga acggaagcag gcaccactaa cttatgaaaa |
| 5821 | ggaggaggaa caggaccaag gggtgagctg caacaaagtg gagtggtggt gggactgctt |
| 5881 | ggagatgctc cgggaaaaca ccttggttac actcgccaac atctcggggc agttggacct |
| 5941 | atctccatac cccgagagca tttgcctgcc tgtcctggac ggactcctac actgggcagt |
| 6001 | ttgcccttca gctgaagccc aggacccctt ttccaccctg ggccccaatg ccgtcctttc |
| 6061 | cccgcagaga ctggtcttgg aaaccctcag caaactcagc atccaggaca acaatgtgga |
| 6121 | cctgattctg gccacacccc ccttcagccg cctggagaag ttgtatagca ctatggtgcg |
| 6181 | cttcctcagt gaccgaaaga acccggtgtg ccgggagatg gctgtggtac tgctggccaa |
| 6241 | cctggctcag ggggacagcc tggcagctcg tgccattgca gtgcagaagg gcagtatcgg |
| 6301 | caacctcctg ggcttcctag aggacagcct tgccgccaca cagttccagc agagccaggc |
| 6361 | cagcctcctc cacatgcaga acccaccctt tgagccaact agtgtggaca tgatgcggcg |
| 6421 | ggctgcccgc gcgctgcttg ccttggccaa ggtggacgag aaccactcag agtttactct |
| 6481 | gtacgaatca cggctgttgg acatctcggt atcaccgttg atgaactcat tggtttcaca |
| 6541 | agtcatttgt gatgtactgt ttttgattgg ccagtcatga cagccgtggg acacctcccc |
| 6601 | cccccgtgtg tgtgtgcgtg tgtggagaac ttagaaactg actgttgccc tttatttatg |
| 6661 | caaaaccacc tcagaatcca gtttaccctg tgctgtccag cttctccctt gggaaaaagt |
| 6721 | ctctcctgtt tctctctcct ccttccacct cccctccctc catcacctca cgcctttctg |
| 6781 | ttccttgtcc tcaccttact cccctcagga ccctacccca ccctctttga aaagacaaag |
| 6841 | ctctgcctac atagaagact ttttttattt taaccaaagt tactgttgtt tacagtgagt |
| 6901 | ttggggaaaa aaaataaaat aaaaatggct ttcccagtcc ttgcatcaac gggatgccac |
| 6961 | atttcataac tgtttttaat ggtaaaaaaa aaaaaaaaaa atacaaaaaa aaattctgaa |
| 7021 | ggacaaaaaa ggtgactgct gaactgtgtg tggtttattg ttgtacattc acaatcttgc |
| 7081 | aggagccaag aagttcgcag ttgtgaacag accctgttca ctggagaggc ctgtgcagta |
| 7141 | gagtgtagac cctttcatgt actgtactgt acacctgata ctgtaaacat actgtaataa |
| 7201 | taatgtctca catggaaaca gaaaacgctg ggtcagcagc aagctgtagt ttttaaaaat |
| 7261 | gtttttagtt aaacgttgag gagaaaaaaa aaaaaggctt ttcccccaaa gtatcatgtg |
| 7321 | tgaacctaca acaccctgac ctctttctct cctccttgat tgtatgaata accctgagat |
| 7381 | cacctcttag aactggtttt aacctttagc tgcagcggct acgctgccac gtgtgtatat |
| 7441 | atatgacgtt gtacattgca catacccttg gatccccaca gtttggtcct cctcccagct |
| 7501 | acccctttat agtatgacga gttaacaagt tggtgacctg cacaaagcga gacacagcta |
| 7561 | tttaatctct tgccagatat cgcccctctt ggtgcgatgc tgtacaggtc tctgtaaaaa |
| 7621 | gtccttgctg tctcagcagc caatcaactt atagtttatt tttttctggg tttttgtttt |
| 7681 | gttttgtttt ctttctaatc gaggtgtgaa aaagttctag gttcagttga agttctgatg |
| 7741 | aagaaacaca attgagattt tttcagtgat aaaatctgca tatttgtatt tcaacaatgt |
| 7801 | agctaaaact tgatgtaaat tcctcctttt tttccttttt tggcttaatg aatatcattt |
| 7861 | attcagtatg aaatctttat actatatgtt ccacgtgtta agaataaatg tacattaaat |
| 7921 | cttggtaaga cttt |

SEQ ID NO: 49 Human ARID1A Amino Acid Sequence isoform B (NP 624361.1)

| | |
|---|---|
| 1 | maaqvapaaa sslgnppppp pselkkaeqq qreeaggeaa aaaaaergem kaaagqeseg |
| 61 | pavgppqplg kelqdgaesn gggggggags gggpgaepdl knsngnagpr palnnnltep |
| 121 | pggggggssd gvgapphsaa aalpppaygf gqpygrspsa vaaaaaavfh qqhggqqspg |
| 181 | laalqsgggg glepyagpqq nshdhgfpnh qynsyypnrs aypppapaya lssprggtpg |
| 241 | sgaaaaagsk pppsssasas sssssfaqqr fgamggggps aagggtpqpt atptinqllt |
| 301 | spssargyqg ypggdysggp qdggagkgpa dmasqcwgaa aaaaaaaaas ggaqqrshha |
| 361 | pmspgssggg gqplartpqp sspmdqmgkm rpqpyggtnp ysqqqgppsg pqqghgypgq |
| 421 | pygsqtpqry pmtmqgraqs amgglsytqq ippygqqgps gygqqgqtpy ynqqsphpqq |
| 481 | qqppysqqpp sqtphaqpsy qqqpqsqppq lassqppysq qpsqpphqqs papypsqqst |
| 541 | tqqhpqsqpp ysqpqaqspy qqqqpqqpap stlsqqaayp qpqsqqsqqt aysqqrfppp |
| 601 | qelsqdsfgs qassapsmts skggqedmnl slqsrpsslp dlsgsiddlp mgtegalspg |
| 661 | vstsgisssq geqsnpaqsp fsphtsphlp girgpspspv gspasvaqsr sgplspaavp |
| 721 | gnqmpprpps gqsdsimhps mnqssiaqdr gymqrnpomp qysspqpgsa lsprqpsggq |
| 781 | ihtgmgsyqq nsmgsygpqg gqygpqggyp rqpnynalpn anypsagmag ginpmgaggq |
| 841 | mhgqpgippy gtlppgrmsh asmgnrpygp nmanmppqvg sgmcpppggm nrktqetava |
| 901 | mhvaansiqn rppgypnmnq ggmmgtgppy gqginsmagm inpqgppysm ggtmannsag |
| 961 | maaspemmgl gdvkltpatk mnnkadgtpk teskskksss stttnekitk lyelggeper |
| 1021 | kmwvdrylaf teekamgmtn lpavgrkpld lyrlyvsvke iggltqvnkn kkwrelatnl |
| 1081 | nvgtsssaas slkkqyiqcl yafeckierg edpppdifaa adskksqpki qppspagsgs |
| 1141 | mqgpqtpqst sssmaeggdl kpptpastph sqipplpgms rsnsvgiqda fndgsdstfq |
| 1201 | krnsmtpnpg yqpsmntsdm mgrmsyepnk dpygsmrkap gsdpfmssgq gpnggmgdpy |
| 1261 | sraagpglgn vamgprqhyp yggpydrvrt epgigpegnm stgapqpnlm psnpdsgmys |
| 1321 | psryppqqqq qqqqrhdsyg nqfstqgtps gspfpsqqtt myqqqqqvss paplprpmen |
| 1381 | rtspskspfl hsgmkmqkag ppvpashiap apvoppmirr ditfppgsve atqpvlkqrr |
| 1441 | rltmkdigtp eawrvmmslk sgllaestwa ldtinillyd dnsimtfnls qlpgllellv |
| 1501 | eyfrrcliei fgilkeyevg dpgqrtlldp grfskvsspa pmeggeeeee llgpkleeee |
| 1561 | eeevvendee iafsgkdkpa senseeklis kfdklpvkiv qkndpfvvdc sdklgrvqef |
| 1621 | dsgllhwrig ggdttehiqt hfesktellp srphapcppa prkhvttaeg tpgttdqegp |
| 1681 | ppdgppekri tatmddmlst rsstltedga ksseaikess kfpfgispaq shrnikiled |
| 1741 | ephskdetpl ctlldwqdsl akrcvcvsnt irslsfvpgn dfemskhpgl llilgklill |
| 1801 | hhkhperkqa pltyekeeeq dqgvsonkve wwwdclemlr entlvtlani sgqldlspyp |
| 1861 | esiclpvldg Ilhwavcpsa eaqdpfstlg pnavlspqrl vletlsklsi qdnnvdlila |
| 1921 | tppfsrlekl ystmvrflsd rknpvcrema vvllanlaqg dslaaraiav qkgsignllg |
| 1981 | fledslaatq fqqsqasllh mqnppfepts vdmmrraara llalakvden hseftlyesr |
| 2041 | lldisvsplm nslvsqvicd vlfligqs |

SEQ ID NO: 50 Mouse ARID1A cDNA Sequence (NM_001080819.1, CDS: from 1
to 6852)

| | |
|---|---|
| 1 | atggccgcgc aggtcgcccc cgccgccgcc agcagcctgg gcaacccgcc gccgccgccc |
| 61 | tcggagctga agaaagccga gcagcaacag cgggaggagg cggggggcga ggcggcggcg |
| 121 | gcagcggccg agcgcgggga aatgaaggca gccgccgggc aggagagcga gggccccgcc |
| 181 | gtggggccgc cgcagccgct gggaaaggag ctgcaggacg gggccgagag caatgggggt |
| 241 | ggcggcggcg gcggagccgg cagcggcggc gggcccggcg cggagccgga cctgaagaac |
| 301 | tcgaacggga acgcgggccc taggcccgcc ctgaacaata acctcccgga gccgcccggc |
| 361 | ggcggcggcg gcggcggcag cagcagcagc gacggggtgg gggcgcctcc tcactcggcc |
| 421 | gcggccgccc tgccgccccc agcctacggc ttcgggcaag cctacggccg gagcccgtct |
| 481 | gccgtcgccg ccgcggcggc cgccgtcttc caccaacaac atggcggaca acaaagccct |
| 541 | ggcctggcag cgctgcagag cggcggcggc gggggcttgg agccctacgc cgggccccag |
| 601 | cagaactcgc acgaccacgg cttccccaac caccagtaca actcctacta ccccaaccgc |
| 661 | agcgcctacc ccccgcctcc ccaggcctac gcgctgagct ccccgagagg tggcactccg |
| 721 | ggctccggcg cggcggcggc cgccggctcc aagccgcctc cctcctccag cgcctctgcc |
| 781 | tcctcgtcgt cttcgtcctt cgcacagcag cgcttcgggg ccatgggggg aggcggcccc |
| 841 | tcagcggccg gcgggggaac tccccagccc accgccaccc ccaccctcaa ccaactgctc |
| 901 | acgtcgccca gctcggcccg tggctaccag ggctaccccg ggggcgacta cggcggcggg |
| 961 | ccccaggacg ggggcgcggg caaaggcccg gcggacatgg cctcgcagtg ctggggggct |
| 1021 | gcggcggcgg cggcggcggc ggcagcggcc gtctcgggag gggcccaaca aaggagccac |
| 1081 | cacgcgccca tgagccccgg gagcagcggc ggcggggggc agccgctcgc ccggacccct |
| 1141 | cagtcatcca gtccaatgga tcagatggga aagatgagac ctcagccgta tggtgggact |
| 1201 | aacccatact cgcaacaaca gggacctcct tcaggaccgc aacaaggaca tgggtaccca |
| 1261 | gggcagccat atgggtccca gactccacag cggtacccca tgaccatgca gggccgggct |
| 1321 | cagagtgcca tgggcagcct ctcttatgca cagcagattc caccttatgg ccagcaaggc |
| 1381 | cccagtgcgt atggccagca gggccagact ccatactata accagcaaag tcctcatccc |
| 1441 | cagcagcagc caccttacgc ccagcaacca ccatcccaga cccctcatgc ccagccttcg |
| 1501 | tatcagcagc agccgcagac tcagcaacca cagcttcagt cctctcagcc tccatattcc |
| 1561 | cagcagccat cccagcctcc acatcagcag tccccaactc catatccctc ccagcagtcc |
| 1621 | accacacaac agcatcccca gagccagccc ccctactcac aaccacaggc acagtctccc |
| 1681 | taccagcagc agcaacctca gcagccagca tcctcgtcgc tctcccagca ggctgcatat |
| 1741 | cctcagcccc agcctcagca gtcccagcaa actgcctatt cccagcagcg cttccctcca |
| 1801 | ccacaggagc tttctcaaga ttcatttggg tctcaggcat cctcagcccc ctcaatgacc |
| 1861 | tccagtaagg gagggcaaga agatatgaac ctgagtcttc agtcaaggcc ctccagcttg |
| 1921 | cctgatctgt ctggttcaat cgatgatctc cccatgggga cagaaggagc tctgagtcct |
| 1981 | ggcgtgagca catcagggat ttccagcagc caaggagagc agagcaatcc agctcagtct |
| 2041 | cccttttctc ctcacacctc ccctcacctg cctggcatcc gaggcccgtc cccgtcccct |
| 2101 | gttggctctc ctgccagtgt cgcgcagtct cgctcaggac cactctcgcc tgctgcagtg |
| 2161 | ccaggcaacc agatgccacc tcggccaccc agtggccagt cagacagcat catgcaccct |
| 2221 | tccatgaacc aatcaagcat tgcccaagat cgaggttata tgcagaggaa cccccagatg |
| 2281 | ccccagtaca cttcccctca gcctggctcg gccttatccc cacgtcagcc gtctggagga |
| 2341 | cagatgcact cgggcgtggg ctcctaccag cagaactcca tggggagcta cggcccccag |
| 2401 | ggcagtcagt atggcccaca aggaggctat cctaggcagc ctaactataa tgccttgccc |
| 2461 | aacgccaact accccaatgc aggcatggcc ggaagtatga accctatggg tgctggaggt |
| 2521 | cagatgcatg ggcagcctgg aatcccacct tacggcacac tccctccagg gagaatggct |
| 2581 | catgcgtcta tgggcaacag gccctatggc cctaatatgg ccaatatgcc acctcaggtt |
| 2641 | gggtcaggga tgtgtcctcc accaggggga atgaacagga aaactcaaga gtctgctgtt |
| 2701 | gccatgcatg ttgctgccaa ctctatccaa aacaggccac caggctaccc aaatatgaat |
| 2761 | caagggggca tgatgggaac tggacctccc tatggacagg ggatcaatag tatggctggc |
| 2821 | atgatcaacc ctcagggacc cccatatcct atgggtggaa ccatggccaa caattcagca |
| 2881 | gggatggcag ccagcccaga gatgatgggc cttggggatg ttaagttaac tcccgccaca |
| 2941 | aaaatgaaca acaaggcaga tggaacaccc aagacagaat ccaaatctaa gaaatccagt |
| 3001 | tcttctacca ccaccaatga gaagatcacc aaattgtatg agttgggtgg tgagcccgag |
| 3061 | aggaagatgt gggtggaccg gtacctggcc ttcacagagg agaaggccat gggcatgaca |
| 3121 | aatctgcctg ctgtggggag gaagcctctg gacctctatc gcctctatgt gtctgtgaag |
| 3181 | gagattggtg ggttgactca ggtcaacaag aacaaaaaat ggcgggaact tgcaaccaac |
| 3241 | ctcaatgtgg gtacatcaag cagtgctgcc agctcactga aaaagcagta tatccaatgt |
| 3301 | ctctatgcct ttgagtgcaa gatcgagcgt ggagaagacc ctccccccga tatcttcgca |
| 3361 | gctgctgact ccaagaagtc ccaacccaag atccagcccc cctctcctgc gggatcaggg |
| 3421 | tctatgcagg ggccacaaac tcctcagtca accagcagtt ctatggcaga aggaggagac |
| 3481 | ctgaagccac caactccagc atccacacca catagtcaaa ttcccccctt accaggcatg |
| 3541 | agcaggagca actcagtcgg aatccaggat gcctttcctg atggaagtga ccccacattc |
| 3601 | cagaagcgga attccatgac tccaaaccct gggtaccagc ccagtatgaa tacctctgac |
| 3661 | atgatggggc gcatgtccta tgagccaaat aaggatcctt atggcagcat gaggaaagcg |
| 3721 | ccaggaagtg atcccttcat gtcctcaggg cagggcccca atggcgggat gggtgatccc |
| 3781 | tacagccgtg ctgctggccc tgggctggga agtgtggcga tgggaccacg gcagcactat |
| 3841 | ccctatggag gtccttacga cagagtgagg acggagcctg gaatcgggcc tgaaggaaat |
| 3901 | atgggcactg gagcccctca gccaaatctc atgccttcca ccccagattc ggggatgtat |
| 3961 | tctcctagcc gctacccccc gcagcagcag cagcaacagc agcaacaaca tgattcctat |
| 4021 | ggcaatcaat tctctaccca aggcacccct tccagcagcc ccttccccag ccagcagacc |
| 4081 | acaatgtatc agcagcagca gcagaattat aagaggccaa tggatggcac atatggcccc |
| 4141 | cctgccaagc ggcatgaagg ggagatgtac agtgtgccgt acagcgctgg gcaaggccag |
| 4201 | cctcaacagc agcagttgcc tgcagctcag tcccagcctg ccagccagcc acaagctgcc |
| 4261 | cagccttccc ctcagcagga cgtgtacaac cagtacagca atgcctaccc tgcctccgcc |
| 4321 | accgctgcta ctgatcgccg accagcaggc ggcccccaga accaatttcc attccagttt |
| 4381 | ggccgagacc gagtctctgc acctcctggt tccagtgccc agcagaacat gccaccacaa |
| 4441 | atgatgggtg gccccataca ggcatcagct gaggttgctc agcagggcac catgtggcag |
| 4501 | gggcgaaatg acatgaccta caattatgcc aacaggcaga acacaggctc tgccacccag |
| 4561 | ggccctgcgt atcatggtgt gaaccgaaca gatgaaatgc tccacacaga tcagagggcc |
| 4621 | aaccatgaag gcccatggcc ttcccatggc acacgccagc ctccgtatgg tccttcagcc |
| 4681 | cctgttcccc ccatgacaag gccccctcca tctaactacc agcccccacc aagcatgccg |
| 4741 | aatcacattc ctcaggtatc cagccccgct cccctccccc ggcccatgga gaaccgtact |
| 4801 | tctcctagca agtctccatt cctgcactct gggatgaaaa tgcaaaaggc gggtccaccg |
| 4861 | gtgcctgctt cgcacatagc gcctacccct gtgcagccgc ctatgattcg gcgggatatc |
| 4921 | accttcccac ctggctctgt agaggccact cagcctgtgt tgaagcagag aaggcggctc |
| 4981 | acaatgaaag acattggaac cccggaggca tggcgggtaa tgatgtccct caagtccggg |
| 5041 | ctcctggcag agagcacgtg ggcgttagac accattaaca ttctactgta tgatgacaac |
| 5101 | agcattatga ccttcaacct cagccagctc ccaggcttgc tagagctcct tgtggaatat |
| 5161 | ttccgtagat gcctaattga aatctttggc attttaaagg agtatgaggt aggggaccca |
| 5221 | ggacagagaa cattactaga ccctgggaga ttcaccaagg tgtatagtcc agcccataca |
| 5281 | gaggaagaag aggaagaaca ccttgatcct aaactggagg aggaagagga agaaggggtt |
| 5341 | ggaaatgatg aggagatggc ctttttgggc aaggacaagc catcttcaga gaataatgag |
| 5401 | gagaagctag tcagtaagtt tgacaagctt ccggtaaaga tcgtgcagag gaatgaccca |
| 5461 | tttgtggtgg actgctcaga taagcttggg cgcgtgcagg agtttgacag tggcctgcta |
| 5521 | cactggcgga ttggtggtgg ggataccact gagcatatcc agacccactt tgagagcaag |
| 5581 | atagagctgc tgccttcccg gccttatgtg ccctgcccaa cgccccctcg gaaacacctc |
| 5641 | acaacagtag agggcacacc agggacaacg gagcaggagg gccccccgcc cgatggcctt |
| 5701 | ccagagaaaa ggatcacagc caccatggat gacatgttgt ctacccggtc tagcacattg |
| 5761 | actgatgagg gggcaaagag tgcagaggcc accaaggaaa gcagcaagtt tccatttggc |
| 5821 | attagcccag cacagagcca ccggaacatc aaaattttag aggatgaacc ccatagtaag |
| 5881 | gatgagaccc cactgtgtac ccttctggac tggcaggatt cccttgctaa gcgctgtgtc |
| 5941 | tgtgtctcca ataccatccg gagcctgtcg tttgtgccag gcaacgactt tgagatgtcc |
| 6001 | aaacacccag ggctgctgct tatcctgggc aagctgatcc tgctgcacca caagcaccca |
| 6061 | gagcggaagc aggcaccact aacttatgag aaggaggagg aacaggacca aggggtgagc |
| 6121 | tgtgacaaag tggagtggtg gtgggactgc ttggagatgc tccgagaaaa cacgctggtc |
| 6181 | accctcgcca acatctcggg gcaattggac ctatccccat atcctgagag catctgcctg |
| 6241 | cctgtcctgg acggactcct acactgggca gtttgccctt cagctgaagc ccaggacccc |
| 6301 | ttctcaaccc taggccccaa tgccgtcctc tccccccaga gattggtctt ggaaaccctc |
| 6361 | agcaaactca gcatccagga caacaatgtg gacctgatcc tggccactcc cccttttagc |
| 6421 | cgcctggaga agttgtatag taccatggtg cgcttcctca gtgaccgaaa gaacccagtg |
| 6481 | tgccgggaga tggccgtggt actgctggca aatctggccc agggggacag cctggcagcc |
| 6541 | cgggccattg cagtgcagaa gggcagcatc ggcaacctcc tgggtttcct ggaggacagc |
| 6601 | cttgctgcca cacagttcca gcagagccag gcaagcctcc tgcatatgca gaatccaccc |
| 6661 | tttgaaccaa ctagtgtgga catgatgcgg cgggctgccc gagcactgct tgccctggcc |
| 6721 | aaggtggatg agaaccactc agagttcact ctgtatgagt cacggctgtt ggacatctcc |
| 6781 | gtgtcaccac tgatgaactc attggtttca caagtcattt gtgatgtact gtttttgatt |
| 6841 | ggccagtcat gacagccgtg ggacacctcc cctccccgtg tgtgtgtgag tgtgtggaga |
| 6901 | acttagaaac tgactgttgc cctttattta tgcaaaacca cctcagaatc cagtttaccc |
| 6961 | tgtgctgtcc agcttctccc ttgggaaagc ctctcctgtt ctctctcctc cccaccctca |
| 7021 | ctccctcaca cctttctgtt ccccatcctc acctgcttcc ctcaggaccc caccctattt |
| 7081 | gaaaagacaa agctctgcct acatagaaga cttttttatt ttaaccaaag ttactgttgt |
| 7141 | ttacagtgag tttggggaaa aaaatggctt tcccagtcct tgcatcaacg ggatgccaca |
| 7201 | tttcataact gtttttaatg gttaaaaaaa aaaaaaaaaa aaggaaaaaa aatacaaaaa |
| 7261 | aaccctgaag gacaaaggtg actgctgagc tgtgtggttt gtcgctgtcc attcacaatc |
| 7321 | tcgcaggagc cgagaagttc gcagttgtga gcagaccctg ttcactggag aggcctgtgc |
| 7381 | agtagagtgt agatcctttc atgtactgta ctgtacacct gatactgtaa acatactgta |
| 7441 | ataataatgt ctcacatgga aacgagagaa gacgctgggt cagcagcaag ctgtagtttt |
| 7501 | taaaaatgtt tttagttaaa tgttgaggag aaaaaaaatg gctttccccc caaagtatcc |
| 7561 | tgtgtgaacc tacaacgccc tgacctcttt ctctcctcct tgattgtatg aatagccctg |
| 7621 | agatcacctc ttagacctgg ttttaacctt tagctgcagc ggctgcgctg ccacgtgtgt |
| 7681 | atatatatga tgttgtacat tgcacatacc cttgaatctc cacagtttgg tccccttccc |
| 7741 | agctacccct ttatagtatg gcgagttaac aagttggtga cctgcacaaa gcgagacaca |
| 7801 | gctatttaat ctcttgccag acattgcccc tcttggtgca gtgctctaca ggtctctgta |
| 7861 | aaaagccctt gctgtctcag cagccaatca acttacagtt tatttttttc tgggtttttg |
| 7921 | ttttgttttg tttcatttct aatcgaggtg tgaaaaagtt ctaggttcag ttgaagttcc |
| 7981 | tgatgaagaa acacaattga gattttttca gtgataaaat ctgcatattt gtatttcaac |
| 8041 | aatgtagcta aaaacttgat gtaaattcct cctttttttt ccttttttgg cttaatgaat |
| 8101 | atcatttatt cagtatgaaa tctttatact atatgttcca cgtgttaaga ataaatgtac |
| 8161 | attaaatctt ggtaa |

SEQ ID NO: 51 Mouse ARID1A Amino Acid Sequence (NP_001074288.1)

| | |
|---|---|
| 1 | maaqvapaaa sslgnppppp selkkaeqqq reeaggeaaa aaaergemka aagqesegpa |
| 61 | vgppqplgke lqdgaesngg gggggagsgg gpgaepdlkn sngnagprpa lnnnlpeppg |
| 121 | ggggggssss dgvgapphsa aaalpppayg fgqaygrsps avaaaaaavf hqqhggqqsp |
| 181 | glaalqsggg gglepyagpq qnshdhgfpn hqynsyypnr sayppppqay alssprggtp |
| 241 | gsgaaaaags kpppsssasa ssssssfaqq rfgamggggp saagggtpqp tatptlnqll |
| 301 | tspssargyq gypggdyggg pqdggagkgp admasqcwga aaaaaaaaaa vsggaqqrsh |
| 361 | hapmspgssg gggqplartp qssspmdqmg kmrpqpyggt npysqqqgpp sgpqqghgyp |
| 421 | gqpygsqtpq rypmtmqgra qsamgslsya qqippygqqg psaygqqgqt pyynqqsphp |
| 481 | qqqppyaqqp psqtphaqps yqqqpqtqqp qlqssqppys qqpsqpphqq sptpypsqqs |
| 541 | ttqqhpqsqp pysqpqaqsp yqqqqpqqpa ssslsqqaay pqpqpqqsqq taysqqrfpp |
| 601 | pqelsqdsfg sqassapsmt sskggqedmn lslqsrpssl pdlsgsiddl pmgtegalsp |
| 661 | gvstsgisss qgeqsnpaqs pfsphtsphl pgirgpspsp vgspasvags rsgplspaav |
| 721 | pgnqmpprpp sgqsdsimhp smnqssiaqd rgymqrnpqm pqytspqpgs alsprqpsgg |
| 781 | qmhsgvgsyq qnsmgsygpq gsqygpqggy prqpnynalp nanypnagma gsmnpmgagg |
| 841 | qmhgqpgipp ygtlppgrma hasmgnrpyg pnmanmppqv gsgmcpppgg mnrktqesav |
| 901 | amhvaansiq nrppgypnmn qggmmgtgpp ygqginsmag minpqgppyp mggtmannsa |
| 961 | gmaaspemmg lgdvkltpat kmnnkadgtp kteskskkss sstttnekit klyelggepe |
| 1021 | rkmwvdryla fteekamgmt nlpavgrkpl dlyrlyvsvk eiggltqvnk nkkwrelatn |
| 1081 | lnvgtsssaa sslkkqyiqc lyafeckier gedpppdifa aadskksqpk iqppspagsg |
| 1141 | smqgpqtpqs tsssmaeggd lkpptpastp hsqipplpgm srsnsvgiqd afpdgsdptf |
| 1201 | qkrnsmtpnp gyqpsmntsd mmgrmsyepn kdpygsmrka pgsdpfmssg qgpnggmgdp |
| 1261 | ysraagpglg svamgprqhy pyggpydrvr tepgigpegn mgtgapqpnl mpstpdsgmy |
| 1321 | spsryppqqq qqqqqqhdsy gnqfstqgtp ssspfpsqqt tmyqqqqqny krpmdgtygp |
| 1381 | pakrhegemy svpysagqgq pqqqqlpaaq sqpasqpqaa qpspqqdvyn qysnaypasa |
| 1441 | taatdrrpag gpqnqfpfqf grdrvsappg ssaqqnmppq mmggpiqasa evaqqgtmwq |
| 1501 | grndmtynya nrqntgsatq gpayhgvnrt demlhtdqra nhegpwpshg trqppygpsa |
| 1561 | pvppmtrppp snyqpppsmp nhipqvsspa plprpmenrt spskspflhs gmkmqkagpp |
| 1621 | vpashiaptp vqppmirrdi tfppgsveat qpvlkqrrrl tmkdigtpea wrvmmslksg |
| 1681 | llaestwald tinillyddn simtfnlsql pgllellvey frrclieifg ilkeyevgdp |
| 1741 | gqrtlldpgr ftkvyspaht eeeeeehldp kleeeeeegv gndeemaflg kdkpssenne |
| 1801 | eklvskfdkl pvkivqrndp fvvdcsdklg rvqefdsgll hwrigggdtt ehiqthfesk |
| 1861 | iellpsrpyv pcptpprkhl ttvegtpgtt eqegpppdgl pekritatmd dmlstrsstl |
| 1921 | tdegaksaea tkesskfpfg ispaqshrni kiledephsk detplctlld wqdslakrcv |
| 1981 | cvsntirsls fvpgndfems khpglllilg klillhhkhp erkqapltye keeeqdqgvs |
| 2041 | cdkvewwwdc lemlrentlv tlanisgqld lspypesicl pvldgllhwa vcpsaeaqdp |
| 2101 | fstlgpnavl spqrlvletl sklsiqdnnv dlilatppfs rleklystmv rflsdrknpv |
| 2161 | cremavvlla nlaqgdslaa raiavqkgsi gnllgfleds laatqfqqsq asllhmqnpp |
| 2221 | feptsvdmmr raarallala kvdenhseft lyesrlldis vsplmnslvs qvicdvlfli |
| 2281 | gqs |

SEQ ID NO: 52 Human ARID1B cDNA Sequence Variant 1 (NM_017519.2, CDS:
from 1 to 6711)

| | |
|---|---|
| 1 | atggcccata acgcgggcgc cgcggccgcc gccggcaccc acagcgccaa gagcggcggc |
| 61 | tccgaggcgg ctctcaagga gggtggaagc gccgccgcgc tgtcctcctc ctcctcctcc |
| 121 | tccgcggcgg cagcggcggc atcctcttcc tcctcgtcgg gcccgggctc ggccatggag |
| 181 | acggggctgc tccccaacca caaactgaaa accgttggcg aagcccccgc cgcgccgccc |
| 241 | caccagcagc accaccacca ccaccatgcc caccaccacc accaccatgc ccaccacctc |
| 301 | caccaccacc acgcactaca gcagcagcta aaccagttcc agcagcagca gcagcagcag |
| 361 | caacagcagc agcagcagca gcagcaacag caacatccca tttccaacaa caacagcttg |
| 421 | ggcggcgcgg gcggcggcgc gcctcagccc ggccccgaca tggagcagcc gcaacatgga |
| 481 | ggcgccaagg acagtgctgc gggcggccag gccgaccccc cgggcccgcc gctgctgagc |
| 541 | aagccgggcg acgaggacga cgcgccgccc aagatggggg agccggcggg cggccgctac |
| 601 | gagcacccgg gcttgggcgc cctgggcacg cagcagccgc cggtcgccgt gcccgggggc |
| 661 | ggcggcggcc cggcggccgt cccggagttt aataattact atggcagcgc tgcccctgcg |
| 721 | agcggcggcc ccggcggccg cgctgggcct tgctttgatc aacatggcgg acaacaaagc |
| 781 | cccgggatgg ggatgatgca ctccgcctcc gccgccgccg ccggggcccc cggcagcatg |
| 841 | gaccccctgc agaactccca cgaagggtac cccaacagcc agtgcaacca ttatccgggc |
| 901 | tacagccggc ccggcgcggg cggcggcggc ggcggcggcg gcggaggagg aggaggcagc |
| 961 | ggaggaggag gaggaggagg aggagcagga gcaggaggag caggagcggg agctgtggcg |
| 1021 | gcggcggccg cggcggcggc ggcagcagca ggaggcggcg gcggcggcgg ctatgggggc |
| 1081 | tcgtccgcgg ggtacggggt gctgagctcc ccccggcagc agggcggcgg catgatgatg |
| 1141 | ggccccgggg gcggcggggc cgcgagcctc agcaaggcgg ccgccggctc ggcggcgggg |
| 1201 | ggcttccagc gcttcgccgg ccagaaccag cacccgtcgg gggccacccc gaccctcaat |
| 1261 | cagctgctca cctcgcccag ccccatgatg cggagctacg gcggcagcta ccccgagtac |
| 1321 | agcagcccca gcgcgccgcc gccgccgccg tcgcagcccc agtcccaggc ggcggcggcg |
| 1381 | ggggcggcgg cgggcggcca gcaggcggcc gcgggcatgg gcttgggcaa ggacatgggc |
| 1441 | gcccagtacg ccgctgccag cccggcctgg gcggccgcgc aacaaaggag tcacccggcg |
| 1501 | atgagccccg gcacccccgg accgaccatg ggcagatccc agggcagccc aatggatcca |
| 1561 | atggtgatga agagacctca gttgtatggc atgggcagta accctcattc tcagcctcag |
| 1621 | cagagcagtc cgtacccagg aggttcctat ggccctccag gcccacagcg gtatccaatt |
| 1681 | ggcatccagg gtcggactcc cggggccatg gccggaatgc agtaccctca gcagcagatg |
| 1741 | ccacctcagt atggacagca aggtgtgagt ggttactgcc agcagggcca acagccatat |
| 1801 | tacagccagc agccgcagcc cccgcacctc ccaccccagg cgcagtatct gccgtcccag |
| 1861 | tcccagcaga ggtaccagcc gcagcaggac atgtctcagg aaggctatgg aactagatct |
| 1921 | caacctcctc tggcccccgg aaaacctaac catgaagact tgaacttaat acagcaagaa |
| 1981 | agaccatcaa gtttaccaga tctgtctggc tccattgatg acctccccac gggaacggaa |
| 2041 | gcaactttga gctcagcagt cagtgcatcc gggtccacga gcagccaagg ggatcagagc |
| 2101 | aacccggcgc agtcgccttt ctccccacat gcgtcccctc atctctccag catcccgggg |
| 2161 | ggcccatctc cctctcctgt tggctctcct gtaggaagca accagtctcg atctggccca |
| 2221 | atctctcctg caagtatccc aggtagtcag atgcctccgc agccacccgg gagccagtca |
| 2281 | gaatccagtt cccatcccgc cttgagccag tcaccaatgc cacaggaaag aggttttatg |
| 2341 | gcaggcacac aaagaaaccc tcagatggct cagtatggac ctcaacagac aggaccatcc |
| 2401 | atgtcgcctc atccttctcc tgggggccag atgcatgctg gaatcagtag ctttcagcag |
| 2461 | agtaactcaa gtgggactta cggtccacag atgagccagt atggaccaca aggtaactac |
| 2521 | tccagacccc cagcgtatag tggggtgccc agtgcaagct acagcggccc agggcccggt |
| 2581 | atgggtatca gtgccaacaa ccagatgcat ggacaagggc caagccagcc atgtggtgct |
| 2641 | gtgcccctgg gacgaatgcc atcagctggg atgcagaaca gaccatttcc tggaaatatg |
| 2701 | agcagcatga cccccagttc tcctggcatg tctcagcagg gagggccagg aatggggccg |
| 2761 | ccaatgccaa ctgtgaaccg taaggcacag gaggcagccg cagcagtgat gcaggctgct |
| 2821 | gcgaactcag cacaaagcag gcaaggcagt ttccccggca tgaaccagag tggacttatg |
| 2881 | gcttccagct ctccctacag ccagcccatg aacaacagct ctagcctgat gaacacgcag |
| 2941 | gcgccgccct acagcatggc gcccgccatg gtgaacagct cggcagcatc tgtgggtctt |
| 3001 | gcagatatga tgtctcctgg tgaatccaaa ctgcccctgc ctctcaaagc agacggcaaa |
| 3061 | gaagaaggca ctccacagcc cgagagcaag tcaaagaagt ccagctcctc caccactact |
| 3121 | ggggagaaga tcacgaaggt gtacgagctg gggaatgagc cagagagaaa gctctgggtc |
| 3181 | gaccgatacc tcaccttcat ggaagagaga ggctctcctg tctcaagtct gcctgccgtg |
| 3241 | ggcaagaagc ccctggacct gttccgactc tacgtctgcg tcaaagagat cgggggtttg |
| 3301 | gcccaggtta ataaaaacaa gaagtggcgt gagctggcaa ccaacctaaa cgttggcacc |
| 3361 | tcaagcagtg cagcgagctc cctgaaaaag cagtatattc agtacctgtt tgcctttgag |
| 3421 | tgcaagatcg aacgtgggga ggagcccccg ccggaagtct tcagcaccgg ggacaccaaa |
| 3481 | aagcagccca agctccagcc gccatctcct gctaactcgg gatccttgca aggcccacag |
| 3541 | accccccagt caactggcag caattccatg gcagaggttc caggtgacct gaagccacct |
| 3601 | accccagcct ccacccctca cggccagatg actccaatgc aaggtggaag aagcagtaca |
| 3661 | atcagtgtgc acgacccatt ctcagatgtg agtgattcat ccttcccgaa acggaactcc |
| 3721 | atgactccaa acgcccccta ccagcagggc atgagcatgc ccgatgtgat gggcaggatg |
| 3781 | ccctatgagc ccaacaagga cccctttggg ggaatgagaa aagtgcctgg aagcagcgag |
| 3841 | ccctttatga cgcaaggaca gatgcccaac agcagcatgc aggacatgta caaccaaagt |
| 3901 | ccctccggag caatgtctaa cctgggcatg gggcagcgcc agcagtttcc ctatggagcc |
| 3961 | agttacgacc gaaggcatga accttatggg cagcagtatc caggccaagg ccctccctcg |
| 4021 | ggacagccgc cgtatggagg gcaccagccc ggcctgtacc cacagcagcc gaattacaaa |
| 4081 | cgccatatgg acggcatgta cgggccccca gccaagcgcc acgagggcga catgtacaac |
| 4141 | atgcagtaca gcagccagca gcaggagatg tacaaccagt atggaggctc ctactcgggc |
| 4201 | ccggaccgca ggcccatcca gggccagtac ccgtatccct acagcaggga gaggatgcag |
| 4261 | ggcccggggc agatccagac acacggaatc ccgcctcaga tgatgggcgg cccgctgcag |
| 4321 | tcgtcctcca gtgaggggcc tcagcagaat atgtgggcag cacgcaatga tatgccttat |
| 4381 | ccctaccaga acaggcaggg ccctggcggc cctacacagg cgccccctta cccaggcatg |
| 4441 | aaccgcacag acgatatgat ggtacccgat cagaggataa atcatgagag ccagtggcct |
| 4501 | tctcacgtca gccagcgtca gccttatatg tcgtcctcag cctccatgca gcccatcaca |
| 4561 | cgcccaccac agccgtccta ccagacgcca ccgtcactgc caaatcacat ctccagggcg |
| 4621 | cccagcccag cgtccttcca gcgctccctg gagaaccgca tgtctccaag caagtctcct |
| 4681 | tttctgccgt ctatgaagat gcagaaggtc atgcccacgg tccccacatc ccaggtcacc |
| 4741 | gggccaccac cccaaccacc cccaatcaga agggagatca cctttcctcc tggctcagta |
| 4801 | gaagcatcac aaccagtctt gaaacaaagg cgaaagatta cctccaaaga tatcgttact |
| 4861 | cctgaggcgt ggcgtgtgat gatgtccctt aaatcaggtc ttttggctga gagtacgtgg |
| 4921 | gctttggaca ctattaatat tcttctgtat gatgacagca ctgttgctac tttcaatctc |
| 4981 | tcccagttgt ctggatttct cgaactttta gtcgagtact ttagaaaatg cctgattgac |
| 5041 | atttttggaa ttcttatgga atatgaagtg ggagacccca gccaaaaagc acttgatcac |
| 5101 | aacgcagcaa ggaaggatga cagccagtcc ttggcagacg attctgggaa agaggaggaa |
| 5161 | gatgctgaat gtattgatga cgacgaggaa gacgaggagg atgaggagga agacagcgag |
| 5221 | aagacagaaa gcgatgaaaa gagcagcatc gctctgactg ccccggacgc cgctgcagac |
| 5281 | ccaaaggaga agcccaagca agccagtaag ttcgacaagc tgccaataaa gatagtcaaa |
| 5341 | aagaacaacc tgtttgttgt tgaccgatct gacaagttgg ggcgtgtgca ggagttcaat |
| 5401 | agtggccttc tgcactggca gctcggcggg ggtgacacca ccgagcacat tcagactcac |
| 5461 | tttgagagca agatggaaat tcctcctcgc aggcgcccac ctcccccctt aagctccgca |
| 5521 | ggtagaaaga aagagcaaga aggcaaaggc gactctgaag agcagcaaga gaaaagcatc |
| 5581 | atagcaacca tcgatgacgt cctctctgct cggccagggg cattgcctga agacgcaaac |
| 5641 | cctgggcccc agaccgaaag cagtaagttt ccctttggta tccagcaagc caaaagtcac |
| 5701 | cggaacatca agctgctgga ggacgagccc aggagccgag acgagactcc tctgtgtacc |
| 5761 | atcgcgcact ggcaggactc gctggctaag cgatgcatct gtgtgtccaa tattgtccgt |
| 5821 | agcttgtcat tcgtgcctgg caatgatgcc gaaatgtcca aacatccagg cctggtgctg |
| 5881 | atcctgggga agctgattct tcttcaccac gagcatccag agagaaagcg agcaccgcag |
| 5941 | acctatgaga aagaggagga tgaggacaag ggggtggcct gcagcaaaga tgagtggtgg |
| 6001 | tgggactgcc tcgaggtctt gagggataac acgttggtca cgttggccaa catttccggg |
| 6061 | cagctagact tgtctgctta cacggaaagc atctgcttgc caattttgga tggcttgctg |
| 6121 | cactggatgg tgtgcccgtc tgcagaggca caagatccct ttccaactgt gggacccaac |
| 6181 | tcggtcctgt cgcctcagag acttgtgctg gagaccctct gtaaactcag tatccaggac |
| 6241 | aataatgtgg acctgatctt ggccactcct ccatttagtc gtcaggagaa attctatgct |
| 6301 | acattagtta ggtacgttgg ggatcgcaaa aacccagtct gtcgagaaat gtccatggcg |
| 6361 | cttttatcga accttgccca aggggacgca ctagcagcaa gggccatagc tgtgcagaaa |
| 6421 | ggaagcattg gaaacttgat aagcttccta gaggatgggg tcacgatggc ccagtaccag |
| 6481 | cagagccagc acaacctcat gcacatgcag cccccgcccc tggaaccacc tagcgtagac |
| 6541 | atgatgtgca gggcggccaa ggctttgcta gccatggcca gagtggacga aaaccgctcg |
| 6601 | gaattccttt tgcacgaggg ccggttgctg gatatctcga tatcagctgt cctgaactct |
| 6661 | ctggttgcat ctgtcatctg tgatgtactg tttcagattg ggcagttatg acataagtga |
| 6721 | gaaggcaagc atgtgtgagt gaagattaga gggtcacata taactggctg ttttctgttc |
| 6781 | ttgtttatcc agcgtaggaa gaaggaaaag aaaatctttg ctcctctgcc ccattcacta |
| 6841 | tttaccaatt gggaattaaa gaaataatta atttgaacag ttatgaaatt aatatttgct |
| 6901 | gtctgtgtgt ataagtacat cctttggggt tttttttttc tctttttttt aaccaaagtt |
| 6961 | gctgtctagt gcattcaaag gtcacttttt gttcttcaca gatcttttta atgttctttc |
| 7021 | ccatgttgta ttgcattttt gggggaagca aattgacttt aaagaaaaaa gttgtggcaa |
| 7081 | aagatgctaa gatgcgaaaa tttcaccaca ctgagtcaaa aaggtgaaaa attatccatt |
| 7141 | tcctatgcgt tttactcctc agagaatgaa aaaaactgca tcccatcacc caaagttctg |
| 7201 | tgcaatagaa atttctacag atacaggtat aggggctcaa ggaggtatgt cggtcagtag |
| 7261 | tcaaaactat gaaatgatac tggtttctcc acaggaatat ggttccatta ggctgggagc |
| 7321 | aaaaacaatg ttttttaaga ttgagaatac atacctgaca acgatccgga aactgctcct |
| 7381 | caccactccc gtcatgcctg ctgtcggcgt ttgaccttcc acgtgacagt tcttcacaat |
| 7441 | tcctttcatc attttttaaa tatttttttt actgcctatg ggctgtgatg tatatagaag |
| 7501 | ttgtacatta aacataccct catttttttc ttttcttttt tttttttttt tttagtacaa |
| 7561 | agttttagtt tctttttcat gatgtggtaa ctacgaagtg atggtagatt taaataattt |
| 7621 | tttattttta ttttatatat tttttcatta gggccatatc tccaaaaaaa gaaagaaaaa |
| 7681 | atacaaaaaa caaaaacaaa aaaaaaagag ggtaatgtac aagtttctgt atgtataaag |
| 7741 | tcatgctcga tttcaggaga gcagctgatc acaatttgct tcatgaatca aggtgtggaa |
| 7801 | atggttatat atggattgat ttagaaaatg gttaccagta cagtcaaaaa agagaaaatg |
| 7861 | aaaaaaatac aactaaaagg aagaaacaca acttcaaaga tttttcagtg atgagaatcc |
| 7921 | acatttgtat ttcaagataa tgtagtttaa aaaaaaaaaa aagaaaaaaa cttgatgtaa |
| 7981 | attcctcctt ttcctctggc ttaatgaata tcatttattc agtataaaat ctttatatgt |
| 8041 | tccacatgtt aagaataaat gtacattaaa tcttgttaag cactgtgatg ggtgttcttg |
| 8101 | aatactgttc tagtttcctt aaagtggttt cctagtaatc aagttattta caagaaatag |
| 8161 | gggaatgcag cagtgtattc acattataaa accctacatt tggaagagac ctttaggggt |
| 8221 | tacctacttt agagtgggga gcaacagttt gattttctca aattacttag ctaattagtc |
| 8281 | tttctttgaa gcaattaact ctaacgacat tgaggtatga tcattttcag tatttatggg |
| 8341 | aggtggctgc tgacccactt gaggtgagat ctcagaagct taactggcct gaaaatgtaa |
| 8401 | cattctgcct tttactaact ccatcttagt ttaatcaaag ttcaatctat tccttgtttc |
| 8461 | ttctgtgtgc ctcagagtta ttttgcattt agtttactcc accgtgtata atatttatac |
| 8521 | tgtgcaatgt taaaaaagaa tctgttatat tgtatgtggt gtacatagtg caaagtgatg |
| 8581 | atttctattt cagggcatat tatggttctc atattccttc ctacctggtg cacagtagct |
| 8641 | ttttaatact agtcacttct aatttaaact ttctcttcct gggtcattga ctgttactgt |
| 8701 | gtaataatcg atttctttga aactgctgca taattatgct gttagtggac ctctacctct |
| 8761 | tctcttccct ctcccaatca cagtatactc agaatcccca gcccctcgca tacattgtgt |
| 8821 | cggttcacat tactcacagt aatatatgga agagttagac aagaacatgc agttacagtc |
| 8881 | attgtgagac gtgactctcc agtgtcacga ggaaaaaaat catcttttct gcaaacagtc |
| 8941 | tctcatctgt caactcccac attactgagt caaacagtct tcttacataa caatgcaacc |
| 9001 | aaatatatgt tgaattaaag acccatttat aattctgctt taaatacatc tgcttgctaa |
| 9061 | gaacagattt cagtgctcca agcttcaaat atggagattt gtaagaggga attcaatatt |
| 9121 | attctaattt ctctcttaca gagtacaaat aaaaggtgta tacaaactcc gaacatatcc |
| 9181 | agtattccaa ttcctttgtc aatcagaaga gtaaaataat taacaaaaga ctgttgttat |
| 9241 | ggtttgcatt gtaaccgata cgcagagtct gaccgttggg caacaagttt ttctatcctg |
| 9301 | atgcgcaaca cagtctctag agactaatcc aggaagactt tagcctcctt tccatattct |
| 9361 | cacccccgaa tcaagattta cagaagccca cgaagaattt acagcctgct tgagatcatc |
| 9421 | ttgcctataa actgagttat tgctttgtcc taaaaattag tcggtttttt tttttctatg |
| 9481 | aggcttttca gaaatttaca ggatgcccag actttacatg tgtaccaaaa aaaaaaaaaa |
| 9541 | gataaaaaat aaaggtgcaa agaaagttta gtattttgga atggtgctat aaagttgaaa |
| 9601 | aaaaaaaaa |

SEQ ID NO: 53 Human ARID1B Amino Acid Sequence isoform A (NP_059989.2)

| | |
|---|---|
| 1 | mahnagaaaa agthsaksgg seaalkeggs aaalssssss saaaaaasss sssgpgsame |
| 61 | tgllpnhklk tvgeapaapp hqqhhhhhha hhhhhhahhl hhhhalqqql ngfqqqqqqq |
| 121 | qqqqqqqqqq qhpisnnnsl ggagggapqp gpdmeqpqhg gakdsaaggq adppgpplls |
| 181 | kpgdeddapp kmgepaggry ehpglgalgt qqppvavpgg gggpaavpef nnyygsaapa |
| 241 | sggpggragp cfdqhggqqs pgmgmmhsas aaaagapgsm dplqnshegy pnsqcnhypg |
| 301 | ysrpgagggg gggggggggs ggggggggag aggagagava aaaaaaaaaa gggggggygg |
| 361 | ssagygvlss prqqgggmmm gpggggaasl skaaagsaag gfqrfagqnq hpsgatptln |
| 421 | qlltspspmm rsyggsypey sspsappppp sqpqsqaaaa gaaaggqqaa agmglgkdmg |
| 481 | aqyaaaspaw aaaqqrshpa mspgtpgptm grsqgspmdp mvmkrpqlyg mgsnphsqpq |
| 541 | qsspypggsy gppgpqrypi giqgrtpgam agmqypqqqm ppqygqqgvs gycqqgqqpy |
| 601 | ysqqpqpphl ppqaqylpsq sqqryqpqqd msqegygtrs qpplapgkpn hedlnliqqe |
| 661 | rpsslpdlsg siddlptgte atlssavsas gstssqgdqs npaqspfsph asphlssipg |
| 721 | gpspspvgsp vgsnqsrsgp ispasipgsq mppqppgsqs essshpalsq spmpqergfm |
| 781 | agtqrnpqma qygpqqtgps msphpspggq mhagissfqq snssgtygpq msqygpqgny |
| 841 | srppaysgvp sasysgpgpg mgisannqmh gqgpsqpcga vplgrmpsag mqnrpfpgnm |
| 901 | ssmtpsspgm sqqggpgmgp pmptvnrkaq eaaaavmqaa ansaqsrqgs fpgmnqsglm |
| 961 | assspysqpm nnssslmntq appysmapam vnssaasvgl admmspgesk lplplkadgk |
| 1021 | eegtpqpesk skkssssttt gekitkvyel gneperklwv dryltfmeer gspvsslpav |
| 1081 | gkkpldlfrl yvcvkeiggl aqvnknkkwr elatnlnvgt sssaasslkk qyiqylfafe |
| 1141 | ckiergeepp pevfstgdtk kqpklqppsp ansgslqgpq tpqstgsnsm aevpgdlkpp |
| 1201 | tpastphgqm tpmqggrsst isvhdpfsdv sdssfpkrns mtpnapyqqg msmpdvmgrm |
| 1261 | pyepnkdpfg gmrkvpgsse pfmtqgqmpn ssmqdmynqs psgamsnlgm gqrqqfpyga |
| 1321 | sydrrhepyg qqypgqgpps gqppygghqp glypqqpnyk rhmdgmygpp akrhegdmyn |
| 1381 | mqyssqqqem ynqyggsysg pdrrpiqgqy pypysrermq gpgqiqthgi ppqmmggplq |
| 1441 | ssssegpqqn mwaarndmpy pygnrqgpgg ptqappypgm nrtddmmvpd qrinhesqwp |
| 1501 | shvsqrqpym sssasmqpit rppqpsyqtp pslpnhisra pspasfqrsl enrmspsksp |
| 1561 | flpsmkmqkv mptvptsqvt gpppqpppir reitfppgsv easqpvlkqr rkitskdivt |
| 1621 | peawrvmmsl ksgllaestw aldtinilly ddstvatfnl sqlsgflell veyfrkclid |
| 1681 | ifgilmeyev gdpsqkaldh naarkddsqs laddsgkeee daecidddee deedeeedse |
| 1741 | ktesdekssi altapdaaad pkekpkqask fdklpikivk knnlfvvdrs dklgrvqefn |
| 1801 | sgllhwqlgg gdttehiqth feskmeippr rrpppplssa grkkeqegkg dseeqqeksi |
| 1861 | iatiddvlsa rpgalpedan pgpqtesskf pfgiqqaksh rniklledep rsrdetplct |
| 1921 | iahwqdslak rcicvsnivr slsfvpgnda emskhpglvl ilgklillhh ehperkrapq |
| 1981 | tyekeededk gvacskdeww wdclevlrdn tlvtlanisg qldlsaytes iclpildgll |
| 2041 | hwmvcpsaea qdpfptvgpn svlspqrlvl etlcklsiqd nnvdlilatp pfsrqekfya |
| 2101 | tlvryvgdrk npvcremsma llsnlaqgda laaraiavqk gsignlisfl edgvtmaqyq |
| 2161 | qsqhnlmhmq pppleppsvd mmcraakall amarvdenrs efllhegrll disisavIns |
| 2221 | lvasvicdvl fqigql |

SEQ ID NO: 54 Human ARID1B cDNA Sequence Variant 2 (NM_020732.3, CDS:
from 1 to 6750)

| | |
|---|---|
| 1 | atggcccata acgcgggcgc cgcggccgcc gccggcaccc acagcgccaa gagcggcggc |
| 61 | tccgaggcgg ctctcaagga gggtggaagc gccgccgcgc tgtcctcctc ctcctcctcc |
| 121 | tccgcggcgg cagcggcggc atcctcttcc tcctcgtcgg gcccgggctc ggccatggag |
| 181 | acggggctgc tccccaacca caaactgaaa accgttggcg aagcccccgc cgcgccgccc |
| 241 | caccagcagc accaccacca ccaccatgcc caccaccacc accaccatgc ccaccacctc |
| 301 | caccaccacc acgcactaca gcagcagcta aaccagttcc agcagcagca gcagcagcag |
| 361 | caacagcagc agcagcagca gcagcaacag caacatccca tttccaacaa caacagcttg |
| 421 | ggcggcgcgg gcggcggcgc gcctcagccc ggccccgaca tggagcagcc gcaacatgga |
| 481 | ggcgccaagg acagtgctgc gggcggccag gccgaccccc cgggcccgcc gctgctgagc |
| 541 | aagccgggcg acgaggacga cgcgccgccc aagatggggg agccggcggg cggccgctac |
| 601 | gagcacccgg gcttgggcgc cctgggcacg cagcagccgc cggtcgccgt gcccgggggc |
| 661 | ggcggcggcc cggcggccgt cccggagttt aataattact atggcagcgc tgcccctgcg |
| 721 | agcggcggcc ccggcggccg cgctgggcct tgctttgatc aacatggcgg acaacaaagc |
| 781 | cccgggatgg ggatgatgca ctccgcctcc gccgccgccg ccggggcccc cggcagcatg |
| 841 | gaccccctgc agaactccca cgaagggtac cccaacagcc agtgcaacca ttatccgggc |
| 901 | tacagccggc ccggcgcggg cggcggcggc ggcggcggcg gcggaggagg aggaggcagc |
| 961 | ggaggaggag gaggaggagg aggagcagga gcaggaggag caggagcggg agctgtggcg |
| 1021 | gcggcggccg cggcggcggc ggcagcagca ggaggcggcg gcggcggcgg ctatgggggc |
| 1081 | tcgtccgcgg ggtacggggt gctgagctcc ccccggcagc agggcggcgg catgatgatg |
| 1141 | ggccccgggg gcggcggggc cgcgagcctc agcaaggcgg ccgccggctc ggcggcgggg |
| 1201 | ggcttccagc gcttcgccgg ccagaaccag cacccgtcgg gggccacccc gaccctcaat |
| 1261 | cagctgctca cctcgcccag ccccatgatg cggagctacg gcggcagcta ccccgagtac |
| 1321 | agcagcccca gcgcgccgcc gccgccgccg tcgcagcccc agtcccaggc ggcggcggcg |
| 1381 | ggggcggcgg cgggcggcca gcaggcggcc gcgggcatgg gcttgggcaa ggacatgggc |
| 1441 | gcccagtacg ccgctgccag cccggcctgg gcggccgcgc aacaaaggag tcacccggcg |
| 1501 | atgagccccg gcacccccgg accgaccatg ggcagatccc agggcagccc aatggatcca |
| 1561 | atggtgatga agagacctca gttgtatggc atgggcagta accctcattc tcagcctcag |
| 1621 | cagagcagtc cgtacccagg aggttcctat ggccctccag gcccacagcg gtatccaatt |
| 1681 | ggcatccagg gtcggactcc cggggccatg gccggaatgc agtaccctca gcagcaggac |
| 1741 | tctggagatg ccacatggaa agaaacattc tggttgatgc cacctcagta tggacagcaa |
| 1801 | ggtgtgagtg gttactgcca gcagggccaa cagccatatt acagccagca gccgcagccc |
| 1861 | ccgcacctcc caccccaggc gcagtatctg ccgtcccagt cccagcagag gtaccagccg |
| 1921 | cagcaggaca tgtctcagga aggctatgga actagatctc aacctcctct ggcccccgga |
| 1981 | aaacctaacc atgaagactt gaacttaata cagcaagaaa gaccatcaag tttaccagat |
| 2041 | ctgtctggct ccattgatga cctccccacg ggaacggaag caactttgag ctcagcagtc |
| 2101 | agtgcatccg ggtccacgag cagccaaggg gatcagagca acccggcgca gtcgcctttc |
| 2161 | tccccacatg cgtcccctca tctctccagc atcccggggg gcccatctcc ctctcctgtt |
| 2221 | ggctctcctg taggaagcaa ccagtctcga tctggcccaa tctctcctgc aagtatccca |
| 2281 | ggtagtcaga tgcctccgca gccacccggg agccagtcag aatccagttc ccatcccgcc |
| 2341 | ttgagccagt caccaatgcc acaggaaaga ggttttatgg caggcacaca aagaaaccct |
| 2401 | cagatggctc agtatggacc tcaacagaca ggaccatcca tgtcgcctca tccttctcct |
| 2461 | gggggccaga tgcatgctgg aatcagtagc tttcagcaga gtaactcaag tgggacttac |
| 2521 | ggtccacaga tgagccagta tggaccacaa ggtaactact ccagaccccc agcgtatagt |
| 2581 | ggggtgccca gtgcaagcta cagcggccca gggcccggta tgggtatcag tgccaacaac |
| 2641 | cagatgcatg gacaagggcc aagccagcca tgtggtgctg tgcccctggg acgaatgcca |
| 2701 | tcagctggga tgcagaacag accatttcct ggaaatatga gcagcatgac ccccagttct |
| 2761 | cctggcatgt ctcagcaggg agggccagga atggggccgc caatgccaac tgtgaaccgt |
| 2821 | aaggcacagg aggcagccgc agcagtgatg caggctgctg cgaactcagc acaaagcagg |
| 2881 | caaggcagtt tccccggcat gaaccagagt ggacttatgg cttccagctc tccctacagc |
| 2941 | cagcccatga acaacagctc tagcctgatg aacacgcagg cgccgcccta cagcatggcg |
| 3001 | cccgccatgg tgaacagctc ggcagcatct gtgggtcttg cagatatgat gtctcctggt |
| 3061 | gaatccaaac tgcccctgcc tctcaaagca gacggcaaag aagaaggcac tccacagccc |
| 3121 | gagagcaagt caaagaagtc cagctcctcc accactactg gggagaagat cacgaaggtg |
| 3181 | tacgagctgg ggaatgagcc agagagaaag ctctgggtcg accgatacct caccttcatg |
| 3241 | gaagagagag gctctcctgt ctcaagtctg cctgccgtgg gcaagaagcc cctggacctg |
| 3301 | ttccgactct acgtctgcgt caaagagatc gggggtttgg cccaggttaa taaaaacaag |
| 3361 | aagtggcgtg agctggcaac caacctaaac gttggcacct caagcagtgc agcgagctcc |
| 3421 | ctgaaaaagc agtatattca gtacctgttt gcctttgagt gcaagatcga acgtggggag |
| 3481 | gagcccccgc cggaagtctt cagcaccggg gacaccaaaa agcagcccaa gctccagccg |
| 3541 | ccatctcctg ctaactcggg atccttgcaa ggcccacaga ccccccagtc aactggcagc |
| 3601 | aattccatgg cagaggttcc aggtgacctg aagccaccta ccccagcctc cacccctcac |
| 3661 | ggccagatga ctccaatgca aggtggaaga agcagtacaa tcagtgtgca cgacccattc |
| 3721 | tcagatgtga gtgattcatc cttcccgaaa cggaactcca tgactccaaa cgccccctac |
| 3781 | cagcagggca tgagcatgcc cgatgtgatg ggcaggatgc cctatgagcc caacaaggac |
| 3841 | ccctttgggg gaatgagaaa agtgcctgga agcagcgagc cctttatgac gcaaggacag |
| 3901 | atgcccaaca gcagcatgca ggacatgtac aaccaaagtc cctccggagc aatgtctaac |
| 3961 | ctgggcatgg ggcagcgcca gcagtttccc tatggagcca gttacgaccg aaggcatgaa |
| 4021 | ccttatgggc agcagtatcc aggccaaggc cctccctcgg gacagccgcc gtatggaggg |
| 4081 | caccagcccg gcctgtaccc acagcagccg aattacaaac gccatatgga cggcatgtac |
| 4141 | gggcccccag ccaagcgcca cgagggcgac atgtacaaca tgcagtacag cagccagcag |
| 4201 | caggagatgt acaaccagta tggaggctcc tactcgggcc cggaccgcag gcccatccag |
| 4261 | ggccagtacc cgtatcccta cagcagggag aggatgcagg gcccggggca gatccagaca |
| 4321 | cacggaatcc cgcctcagat gatgggcggc ccgctgcagt cgtcctccag tgaggggcct |
| 4381 | cagcagaata tgtgggcagc acgcaatgat atgccttatc cctaccagaa caggcagggc |
| 4441 | cctggcggcc ctacacaggc gcccccttac ccaggcatga accgcacaga cgatatgatg |
| 4501 | gtacccgatc agaggataaa tcatgagagc cagtggcctt ctcacgtcag ccagcgtcag |
| 4561 | ccttatatgt cgtcctcagc ctccatgcag cccatcacac gcccaccaca gccgtcctac |
| 4621 | cagacgccac cgtcactgcc aaatcacatc tccagggcgc ccagcccagc gtccttccag |
| 4681 | cgctccctgg agaaccgcat gtctccaagc aagtctcctt ttctgccgtc tatgaagatg |
| 4741 | cagaaggtca tgcccacggt ccccacatcc caggtcaccg ggccaccacc ccaaccaccc |
| 4801 | ccaatcagaa gggagatcac ctttcctcct ggctcagtag aagcatcaca accagtcttg |
| 4861 | aaacaaaggc gaaagattac ctccaaagat atcgttactc ctgaggcgtg gcgtgtgatg |
| 4921 | atgtccctta aatcaggtct tttggctgag agtacgtggg ctttggacac tattaatatt |
| 4981 | cttctgtatg atgacagcac tgttgctact ttcaatctct cccagttgtc tggatttctc |
| 5041 | gaacttttag tcgagtactt tagaaaatgc ctgattgaca tttttggaat tcttatggaa |
| 5101 | tatgaagtgg gagaccccag ccaaaaagca cttgatcaca acgcagcaag gaaggatgac |
| 5161 | agccagtcct tggcagacga ttctgggaaa gaggaggaag atgctgaatg tattgatgac |
| 5221 | gacgaggaag acgaggagga tgaggaggaa gacagcgaga agacagaaag cgatgaaaag |
| 5281 | agcagcatcg ctctgactgc cccggacgcc gctgcagacc caaaggagaa gcccaagcaa |
| 5341 | gccagtaagt tcgacaagct gccaataaag atagtcaaaa agaacaacct gtttgttgtt |
| 5401 | gaccgatctg acaagttggg gcgtgtgcag gagttcaata gtggccttct gcactggcag |
| 5461 | ctcggcgggg gtgacaccac cgagcacatt cagactcact ttgagagcaa gatggaaatt |
| 5521 | cctcctcgca ggcgcccacc tcccccctta agctccgcag gtagaaagaa agagcaagaa |
| 5581 | ggcaaaggcg actctgaaga gcagcaagag aaaagcatca tagcaaccat cgatgacgtc |
| 5641 | ctctctgctc ggccaggggc attgcctgaa gacgcaaacc ctgggcccca gaccgaaagc |
| 5701 | agtaagtttc cctttggtat ccagcaagcc aaaagtcacc ggaacatcaa gctgctggag |
| 5761 | gacgagccca ggagccgaga cgagactcct ctgtgtacca tcgcgcactg gcaggactcg |
| 5821 | ctggctaagc gatgcatctg tgtgtccaat attgtccgta gcttgtcatt cgtgcctggc |
| 5881 | aatgatgccg aaatgtccaa acatccaggc ctggtgctga tcctggggaa gctgattctt |
| 5941 | cttcaccacg agcatccaga gagaaagcga gcaccgcaga cctatgagaa agaggaggat |
| 6001 | gaggacaagg gggtggcctg cagcaaagat gagtggtggt gggactgcct cgaggtcttg |
| 6061 | agggataaca cgttggtcac gttggccaac atttccgggc agctagactt gtctgcttac |
| 6121 | acggaaagca tctgcttgcc aattttggat ggcttgctgc actggatggt gtgcccgtct |
| 6181 | gcagaggcac aagatccctt tccaactgtg ggacccaact cggtcctgtc gcctcagaga |
| 6241 | cttgtgctgg agaccctctg taaactcagt atccaggaca ataatgtgga cctgatcttg |
| 6301 | gccactcctc catttagtcg tcaggagaaa ttctatgcta cattagttag gtacgttggg |
| 6361 | gatcgcaaaa acccagtctg tcgagaaatg tccatggcgc ttttatcgaa ccttgcccaa |
| 6421 | ggggacgcac tagcagcaag ggccatagct gtgcagaaag gaagcattgg aaacttgata |
| 6481 | agcttcctag aggatggggt cacgatggcc cagtaccagc agagccagca caacctcatg |
| 6541 | cacatgcagc ccccgcccct ggaaccacct agcgtagaca tgatgtgcag ggcggccaag |
| 6601 | gctttgctag ccatggccag agtggacgaa aaccgctcgg aattcctttt gcacgagggc |
| 6661 | cggttgctgg atatctcgat atcagctgtc ctgaactctc tggttgcatc tgtcatctgt |
| 6721 | gatgtactgt ttcagattgg gcagttatga cataagtgag aaggcaagca tgtgtgagtg |
| 6781 | aagattagag ggtcacatat aactggctgt tttctgttct tgtttatcca gcgtaggaag |
| 6841 | aaggaaaaga aaatctttgc tcctctgccc cattcactat ttaccaattg ggaattaaag |
| 6901 | aaataattaa tttgaacagt tatgaaatta atatttgctg tctgtgtgta taagtacatc |
| 6961 | ctttggggtt ttttttttct ctttttttta accaaagttg ctgtctagtg cattcaaagg |
| 7021 | tcactttttg ttcttcacag atctttttaa tgttctttcc catgttgtat tgcatttttg |
| 7081 | ggggaagcaa attgacttta aagaaaaaag ttgtggcaaa agatgctaag atgcgaaaat |
| 7141 | ttcaccacac tgagtcaaaa aggtgaaaaa ttatccattt cctatgcgtt ttactcctca |
| 7201 | gagaatgaaa aaaactgcat cccatcaccc aaagttctgt gcaatagaaa tttctacaga |
| 7261 | tacaggtata ggggctcaag gaggtatgtc ggtcagtagt caaaactatg aaatgatact |
| 7321 | ggtttctcca caggaatatg gttccattag gctgggagca aaaacaatgt tttttaagat |
| 7381 | tgagaataca tacctgacaa cgatccggaa actgctcctc accactcccg tcatgcctgc |
| 7441 | tgtcggcgtt tgaccttcca cgtgacagtt cttcacaatt cctttcatca ttttttaaat |
| 7501 | atttttttta ctgcctatgg gctgtgatgt atatagaagt tgtacattaa acataccctc |
| 7561 | atttttttct tttctttttt tttttttttt ttagtacaaa gttttagttt ctttttcatg |
| 7621 | atgtggtaac tacgaagtga tggtagattt aaataatttt ttatttttat tttatatatt |
| 7681 | ttttcattag ggccatatct ccaaaaaaag aaagaaaaaa tacaaaaaac aaaaacaaaa |
| 7741 | aaaaaagagg gtaatgtaca agtttctgta tgtataaagt catgctcgat ttcaggagag |
| 7801 | cagctgatca caatttgctt catgaatcaa ggtgtggaaa tggttatata tggattgatt |
| 7861 | tagaaaatgg ttaccagtac agtcaaaaaa gagaaaatga aaaaaataca actaaaagga |
| 7921 | agaaacacaa cttcaaagat ttttcagtga tgagaatcca catttgtatt tcaagataat |
| 7981 | gtagtttaaa aaaaaaaaaa agaaaaaaac ttgatgtaaa ttcctccttt tcctctggct |
| 8041 | taatgaatat catttattca gtataaaatc tttatatgtt ccacatgtta agaataaatg |
| 8101 | tacattaaat cttgttaagc actgtgatgg gtgttcttga atactgttct agtttcctta |
| 8161 | aagtggtttc ctagtaatca agttatttac aagaaatagg ggaatgcagc agtgtattca |
| 8221 | cattataaaa ccctacattt ggaagagacc tttaggggtt acctacttta gagtggggag |
| 8281 | caacagtttg attttctcaa attacttagc taattagtct ttctttgaag caattaactc |
| 8341 | taacgacatt gaggtatgat cattttcagt atttatggga ggtggctgct gacccacttg |
| 8401 | aggtgagatc tcagaagctt aactggcctg aaaatgtaac attctgcctt ttactaactc |
| 8461 | catcttagtt taatcaaagt tcaatctatt ccttgtttct tctgtgtgcc tcagagttat |
| 8521 | tttgcattta gtttactcca ccgtgtataa tatttatact gtgcaatgtt aaaaaagaat |
| 8581 | ctgttatatt gtatgtggtg tacatagtgc aaagtgatga tttctatttc agggcatatt |
| 8641 | atggttctca tattccttcc tacctggtgc acagtagctt tttaatacta gtcacttcta |
| 8701 | atttaaactt tctcttcctg ggtcattgac tgttactgtg taataatcga tttctttgaa |
| 8761 | actgctgcat aattatgctg ttagtggacc tctacctctt ctcttccctc tcccaatcac |
| 8821 | agtatactca gaatccccag cccctcgcat acattgtgtc ggttcacatt actcacagta |
| 8881 | atatatggaa gagttagaca agaacatgca gttacagtca ttgtgagacg tgactctcca |
| 8941 | gtgtcacgag gaaaaaaatc atcttttctg caaacagtct ctcatctgtc aactcccaca |
| 9001 | ttactgagtc aaacagtctt cttacataac aatgcaacca aatatatgtt gaattaaaga |
| 9061 | cccatttata attctgcttt aaatacatct gcttgctaag aacagatttc agtgctccaa |
| 9121 | gcttcaaata tggagatttg taagagggaa ttcaatatta ttctaatttc tctcttacag |
| 9181 | agtacaaata aaaggtgtat acaaactccg aacatatcca gtattccaat tcctttgtca |
| 9241 | atcagaagag taaaataatt aacaaaagac tgttgttatg gtttgcattg taaccgatac |
| 9301 | gcagagtctg accgttgggc aacaagtttt tctatcctga tgcgcaacac agtctctaga |
| 9361 | gactaatcca ggaagacttt agcctccttt ccatattctc acccccgaat caagatttac |
| 9421 | agaagcccac gaagaattta cagcctgctt gagatcatct tgcctataaa ctgagttatt |
| 9481 | gctttgtcct aaaaattagt cggttttttt ttttctatga ggcttttcag aaatttacag |
| 9541 | gatgcccaga ctttacatgt gtaccaaaaa aaaaaaaaag ataaaaaata aaggtgcaaa |
| 9601 | gaaagtttag tattttggaa tggtgctata aagttgaaaa aaaaaaaa |

SEQ ID NO: 55 Human ARID1B Amino Acid Sequence isoform B (NP_065783.3)

| | |
|---|---|
| 1 | mahnagaaaa agthsaksgg seaalkeggs aaalssssss saaaaaasss sssgpgsame |
| 61 | tgllpnhklk tvgeapaapp hqqhhhhhha hhhhhhahhl hhhhalqqql ngfqqqqqqq |
| 121 | qqqqqqqqqq qhpisnnnsl ggagggapqp gpdmeqpqhg gakdsaaggq adppgpplls |
| 181 | kpgdeddapp kmgepaggry ehpglgalgt qqppvavpgg gggpaavpef nnyygsaapa |
| 241 | sggpggragp cfdqhggqqs pgmgmmhsas aaaagapgsm dplqnshegy pnsqcnhypg |
| 301 | ysrpgagggg gggggggggs ggggggggag aggagagava aaaaaaaaaa gggggggygg |
| 361 | ssagygvlss prqqgggmmm gpggggaasl skaaagsaag gfqrfagqnq hpsgatptln |
| 421 | qlltspspmm rsyggsypey sspsappppp sqpqsqaaaa gaaaggqqaa agmglgkdmg |
| 481 | aqyaaaspaw aaaqqrshpa mspgtpgptm grsqgspmdp mvmkrpqlyg mgsnphsqpq |
| 541 | qsspypggsy gppgpqrypi giqgrtpgam agmqypqqqd sgdatwketf wlmppqygqq |
| 601 | gvsgycqqgq qpyysqqpqp phlppqaqyl psqsqqryqp qqdmsqegyg trsqpplapg |
| 661 | kpnhedlnli qqerpsslpd lsgsiddlpt gteatlssav sasgstssqg dqsnpaqspf |
| 721 | sphasphlss ipggpspspv gspvgsnqsr sgpispasip gsqmppqppg sqsessshpa |
| 781 | lsqspmpqer gfmagtqrnp qmagygpqqt gpsmsphpsp ggqmhagiss fqqsnssgty |
| 841 | gpqmsqygpq gnysrppays gvpsasysgp gpgmgisann qmhgqgpsqp cgavplgrmp |
| 901 | sagmqnrpfp gnmssmtpss pgmsqqggpg mgppmptvnr kaqeaaaavm qaaansaqsr |
| 961 | qgsfpgmnqs glmassspys qpmnnssslm ntqappysma pamvnssaas vgladmmspg |
| 1021 | esklplplka dgkeegtpqp eskskkssss tttgekitkv yelgneperk lwvdryltfm |
| 1081 | eergspvssl pavgkkpldl frlyvcvkei gglaqvnknk kwrelatnln vgtsssaass |
| 1141 | lkkqyiqylf afeckierge epppevfstg dtkkqpklqp pspansgslq gpqtpqstgs |
| 1201 | nsmaevpgdl kpptpastph gqmtpmqggr sstisvhdpf sdvsdssfpk rnsmtpnapy |
| 1261 | qqgmsmpdvm grmpyepnkd pfggmrkvpg ssepfmtqgq mpnssmqdmy nqspsgamsn |
| 1321 | 1gmgqrqqfp ygasydrrhe pygqqypgqg ppsgqppygg hqpglypqqp nykrhmdgmy |
| 1381 | gppakrhegd mynmqyssqq qemynqyggs ysgpdrrpiq gqypypysre rmqgpgqiqt |
| 1441 | hgippqmmgg plqssssegp qqnmwaarnd mpypyqnrqg pggptqappy pgmnrtddmm |
| 1501 | vpdqrinhes qwpshvsqrq pymsssasmq pitrppqpsy qtppslpnhi srapspasfq |
| 1561 | rslenrmsps kspflpsmkm qkvmptvpts qvtgpppqpp pirreitfpp gsveasqpvl |
| 1621 | kqrrkitskd ivtpeawrvm mslksgllae stwaldtini llyddstvat fnlsqlsgfl |
| 1681 | ellveyfrkc lidifgilme yevgdpsqka ldhnaarkdd sqsladdsgk eeedaecidd |
| 1741 | deedeedeee dsektesdek ssialtapda aadpkekpkq askfdklpik ivkknnlfvv |
| 1801 | drsdklgrvq efnsgllhwq lgggdttehi qthfeskmei pprrrppppl ssagrkkeqe |
| 1861 | gkgdseeqqe ksiiatiddv lsarpgalpe danpgpqtes skfpfgiqqa kshrniklle |
| 1921 | deprsrdetp lctiahwqds lakrcicvsn ivrslsfvpg ndaemskhpg lvlilgklil |
| 1981 | lhhehperkr apqtyekeed edkgvacskd ewwwdclevl rdntlvtlan isgqldlsay |
| 2041 | tesiclpild gllhwmvcps aeaqdpfptv gpnsvlspqr lvletlckls iqdnnvdlil |
| 2101 | atppfsrqek fyatlvryvg drknpvcrem smallsnlaq gdalaaraia vqkgsignli |
| 2161 | sfledgvtma qyqqsqhnlm hmqppplepp svdmmcraak allamarvde nrsefllheg |
| 2221 | rlldisisav lnslvasvic dvlfqigql |

SEQ ID NO: 56 Human ARID1B cDNA Sequence Variant 3 (NM_001346813.1,
CDS: from 76 to 6945)

| | |
|---|---|
| 1 | gggggcggcg gcgacggcgg cggcggcctg aacagtgtgc accaccaccc cctgctcccc |
| 61 | cgtcacgaac tcaacatggc ccataacgcg ggcgccgcgg ccgccgccgg cacccacagc |
| 121 | gccaagagcg gcggctccga ggcggctctc aaggagggtg gaagcgccgc cgcgctgtcc |
| 181 | tcctcctcct cctcctccgc ggcggcagcg gcggcatcct cttcctcctc gtcgggcccg |
| 241 | ggctcggcca tggagacggg gctgctcccc aaccacaaac tgaaaaccgt tggcgaagcc |
| 301 | cccgccgcgc cgccccacca gcagcaccac caccaccacc atgcccacca ccaccaccac |
| 361 | catgcccacc acctccacca ccaccacgca ctacagcagc agctaaacca gttccagcag |
| 421 | cagcagcagc agcagcaaca gcagcagcag cagcagcagc aacagcaaca tcccatttcc |
| 481 | aacaacaaca gcttgggcgg cgcgggcggc ggcgcgcctc agcccggccc cgacatggag |
| 541 | cagccgcaac atggaggcgc caaggacagt gctgcgggcg gccaggccga ccccccgggc |
| 601 | ccgccgctgc tgagcaagcc gggcgacgag gacgacgcgc cgcccaagat gggggagccg |
| 661 | gcgggcggcc gctacgagca cccgggcttg ggcgccctgg gcacgcagca gccgccggtc |
| 721 | gccgtgcccg ggggcggcgg cggcccggcg gccgtcccgg agtttaataa ttactatggc |
| 781 | agcgctgccc ctgcgagcgg cggccccggc ggccgcgctg ggccttgctt tgatcaacat |
| 841 | ggcggacaac aaagccccgg gatggggatg atgcactccg cctccgccgc cgccgccggg |
| 901 | gcccccggca gcatggaccc cctgcagaac tcccacgaag ggtaccccaa cagccagtgc |
| 961 | aaccattatc cgggctacag ccggcccggc gcgggcggcg gcggcggcgg cggcggcgga |
| 1021 | ggaggaggag gcagcggagg aggaggagga ggaggaggag caggagcagg aggagcagga |
| 1081 | gcgggagctg tggcggcggc ggccgcggcg gcggcggcag cagcaggagg cggcggcggc |
| 1141 | ggcggctatg ggggctcgtc cgcggggtac ggggtgctga gctccccccg gcagcagggc |
| 1201 | ggcggcatga tgatgggccc cgggggcggc ggggccgcga gcctcagcaa ggcggccgcc |
| 1261 | ggctcggcgg cggggggctt ccagcgcttc gccggccaga accagcaccc gtcgggggcc |
| 1321 | accccgaccc tcaatcagct gctcacctcg cccagcccca tgatgcggag ctacggcggc |
| 1381 | agctaccccg agtacagcag ccccagcgcg ccgccgccgc cgccgtcgca gccccagtcc |
| 1441 | caggcggcgg cggcgggggc ggcggcgggc ggccagcagg cggccgcggg catgggcttg |
| 1501 | ggcaaggaca tgggcgccca gtacgccgct gccagcccgg cctgggcggc cgcgcaacaa |
| 1561 | aggagtcacc cggcgatgag ccccggcacc cccggaccga ccatgggcag atcccagggc |
| 1621 | agcccaatgg atccaatggt gatgaagaga cctcagttgt atggcatggg cagtaaccct |
| 1681 | cattctcagc ctcagcagag cagtccgtac ccaggaggtt cctatggccc tccaggccca |
| 1741 | cagcggtatc caattggcat ccagggtcgg actcccgggg ccatggccgg aatgcagtac |
| 1801 | cctcagcagc agatgccacc tcagtatgga cagcaaggtg tgagtggtta ctgccagcag |
| 1861 | ggccaacagc catattacag ccagcagccg cagcccccgc acctcccacc ccaggcgcag |
| 1921 | tatctgccgt cccagtccca gcagaggtac cagccgcagc aggacatgtc tcaggaaggc |
| 1981 | tatggaacta gatctcaacc tcctctggcc cccggaaaac ctaaccatga agacttgaac |
| 2041 | ttaatacagc aagaaagacc atcaagttta ccagatctgt ctggctccat tgatgacctc |
| 2101 | cccacgggaa cggaagcaac tttgagctca gcagtcagtg catccgggtc cacgagcagc |
| 2161 | caaggggatc agagcaaccc ggcgcagtcg cctttctccc cacatgcgtc ccctcatctc |
| 2221 | tccagcatcc cggggggccc atctccctct cctgttggct ctcctgtagg aagcaaccag |
| 2281 | tctcgatctg gcccaatctc tcctgcaagt atcccaggta gtcagatgcc tccgcagcca |
| 2341 | cccgggagcc agtcagaatc cagttcccat cccgccttga gccagtcacc aatgccacag |
| 2401 | gaaagaggtt ttatggcagg cacacaaaga aaccctcaga tggctcagta tggacctcaa |
| 2461 | cagacaggac catccatgtc gcctcatcct tctcctgggg gccagatgca tgctggaatc |
| 2521 | agtagctttc agcagagtaa ctcaagtggg acttacggtc cacagatgag ccagtatgga |
| 2581 | ccacaaggta actactccag acccccagcg tatagtgggg tgcccagtgc aagctacagc |
| 2641 | ggcccagggc ccggtatggg tatcagtgcc aacaaccaga tgcatggaca agggccaagc |
| 2701 | cagccatgtg gtgctgtgcc cctgggacga atgccatcag ctgggatgca gaacagacca |
| 2761 | tttcctggaa atatgagcag catgaccccc agttctcctg gcatgtctca gcagggaggg |
| 2821 | ccaggaatgg ggccgccaat gccaactgtg aaccgtaagg cacaggaggc agccgcagca |
| 2881 | gtgatgcagg ctgctgcgaa ctcagcacaa agcaggcaag gcagtttccc cggcatgaac |
| 2941 | cagagtggac ttatggcttc cagctctccc tacagccagc ccatgaacaa cagctctagc |
| 3001 | ctgatgaaca cgcaggcgcc gccctacagc atggcgcccg ccatggtgaa cagctcggca |
| 3061 | gcatctgtgg gtcttgcaga tatgatgtct cctggtgaat ccaaactgcc cctgcctctc |
| 3121 | aaagcagacg gcaaagaaga aggcactcca cagcccgaga gcaagtcaaa ggatagctac |
| 3181 | agctctcagg gtatttctca gcccccaacc ccaggcaacc tgccagtccc ttccccaatg |
| 3241 | tcccccagct ctgctagcat ctcctcattt catggagatg aaagtgatag cattagcagc |
| 3301 | ccaggctggc caaagactcc atcaagccct aagtccagct cctccaccac tactggggag |
| 3361 | aagatcacga aggtgtacga gctggggaat gagccagaga gaaagctctg ggtcgaccga |
| 3421 | tacctcacct tcatggaaga gagaggctct cctgtctcaa gtctgcctgc cgtgggcaag |
| 3481 | aagcccctgg acctgttccg actctacgtc tgcgtcaaag agatcggggg tttggcccag |
| 3541 | gttaataaaa acaagaagtg gcgtgagctg gcaaccaacc taaacgttgg cacctcaagc |
| 3601 | agtgcagcga gctccctgaa aaagcagtat attcagtacc tgtttgcctt tgagtgcaag |
| 3661 | atcgaacgtg gggaggagcc cccgccggaa gtcttcagca ccggggacac caaaaagcag |
| 3721 | cccaagctcc agccgccatc tcctgctaac tcgggatcct tgcaaggccc acagaccccc |
| 3781 | cagtcaactg gcagcaattc catggcagag gttccaggtg acctgaagcc acctacccca |
| 3841 | gcctccaccc ctcacggcca gatgactcca atgcaaggtg gaagaagcag tacaatcagt |
| 3901 | gtgcacgacc cattctcaga tgtgagtgat tcatccttcc cgaaacggaa ctccatgact |
| 3961 | ccaaacgccc cctaccagca gggcatgagc atgcccgatg tgatgggcag gatgccctat |
| 4021 | gagcccaaca aggacccctt tgggggaatg agaaaagtgc ctggaagcag cgagcccttt |
| 4081 | atgacgcaag gacagatgcc caacagcagc atgcaggaca tgtacaacca aagtccctcc |
| 4141 | ggagcaatgt ctaacctggg catggggcag cgccagcagt ttccctatgg agccagttac |
| 4201 | gaccgaaggc atgaacctta tgggcagcag tatccaggcc aaggccctcc ctcgggacag |
| 4261 | ccgccgtatg gagggcacca gcccggcctg tacccacagc agccgaatta caaacgccat |
| 4321 | atggacggca tgtacgggcc cccagccaag cgccacgagg gcgacatgta caacatgcag |
| 4381 | tacagcagcc agcagcagga gatgtacaac cagtatggag gctcctactc gggcccggac |
| 4441 | cgcaggccca tccagggcca gtacccgtat ccctacagca gggagaggat gcagggcccg |
| 4501 | gggcagatcc agacacacgg aatcccgcct cagatgatgg gcggcccgct gcagtcgtcc |
| 4561 | tccagtgagg ggcctcagca gaatatgtgg gcagcacgca atgatatgcc ttatccctac |
| 4621 | cagaacaggc agggccctgg cggccctaca caggcgcccc cttacccagg catgaaccgc |
| 4681 | acagacgata tgatggtacc cgatcagagg ataaatcatg agagccagtg gccttctcac |
| 4741 | gtcagccagc gtcagcctta tatgtcgtcc tcagcctcca tgcagcccat cacacgccca |
| 4801 | ccacagccgt cctaccagac gccaccgtca ctgccaaatc acatctccag ggcgcccagc |
| 4861 | ccagcgtcct tccagcgctc cctggagaac cgcatgtctc caagcaagtc tccttttctg |
| 4921 | ccgtctatga agatgcagaa ggtcatgccc acggtcccca catcccaggt caccgggcca |
| 4981 | ccaccccaac cacccccaat cagaagggag atcacctttc ctcctggctc agtagaagca |
| 5041 | tcacaaccag tcttgaaaca aaggcgaaag attacctcca aagatatcgt tactcctgag |
| 5101 | gcgtggcgtg tgatgatgtc ccttaaatca ggtcttttgg ctgagagtac gtgggctttg |
| 5161 | gacactatta atattcttct gtatgatgac agcactgttg ctactttcaa tctctcccag |
| 5221 | ttgtctggat ttctcgaact tttagtcgag tactttagaa aatgcctgat tgacattttt |
| 5281 | ggaattctta tggaatatga agtgggagac cccagccaaa aagcacttga tcacaacgca |
| 5341 | gcaaggaagg atgacagcca gtccttggca gacgattctg ggaaagagga ggaagatgct |
| 5401 | gaatgtattg atgacgacga ggaagacgag gaggatgagg aggaagacag cgagaagaca |
| 5461 | gaaagcgatg aaaagagcag catcgctctg actgccccgg acgccgctgc agacccaaag |
| 5521 | gagaagccca agcaagccag taagttcgac aagctgccaa taaagatagt caaaaagaac |
| 5581 | aacctgtttg ttgttgaccg atctgacaag ttggggcgtg tgcaggagtt caatagtggc |
| 5641 | cttctgcact ggcagctcgg cgggggtgac accaccgagc acattcagac tcactttgag |
| 5701 | agcaagatgg aaattcctcc tcgcaggcgc ccacctcccc ccttaagctc cgcaggtaga |
| 5761 | aagaaagagc aagaaggcaa aggcgactct gaagagcagc aagagaaaag catcatagca |
| 5821 | accatcgatg acgtcctctc tgctcggcca ggggcattgc ctgaagacgc aaaccctggg |
| 5881 | ccccagaccg aaagcagtaa gtttcccttt ggtatccagc aagccaaaag tcaccggaac |
| 5941 | atcaagctgc tggaggacga gcccaggagc cgagacgaga ctcctctgtg taccatcgcg |
| 6001 | cactggcagg actcgctggc taagcgatgc atctgtgtgt ccaatattgt ccgtagcttg |
| 6061 | tcattcgtgc ctggcaatga tgccgaaatg tccaaacatc caggcctggt gctgatcctg |
| 6121 | gggaagctga ttcttcttca ccacgagcat ccagagagaa agcgagcacc gcagacctat |
| 6181 | gagaaagagg aggatgagga caagggggtg gcctgcagca aagatgagtg gtggtgggac |
| 6241 | tgcctcgagg tcttgaggga taacacgttg gtcacgttgg ccaacatttc cgggcagcta |
| 6301 | gacttgtctg cttacacgga aagcatctgc ttgccaattt tggatggctt gctgcactgg |
| 6361 | atggtgtgcc cgtctgcaga ggcacaagat ccctttccaa ctgtgggacc caactcggtc |
| 6421 | ctgtcgcctc agagacttgt gctggagacc ctctgtaaac tcagtatcca ggacaataat |
| 6481 | gtggacctga tcttggccac tcctccattt agtcgtcagg agaaattcta tgctacatta |
| 6541 | gttaggtacg ttggggatcg caaaaaccca gtctgtcgag aaatgtccat ggcgctttta |
| 6601 | tcgaaccttg cccaagggga cgcactagca gcaagggcca tagctgtgca gaaaggaagc |
| 6661 | attggaaact tgataagctt cctagaggat ggggtcacga tggcccagta ccagcagagc |
| 6721 | cagcacaacc tcatgcacat gcagcccccg cccctggaac cacctagcgt agacatgatg |
| 6781 | tgcagggcgg ccaaggcttt gctagccatg gccagagtgg acgaaaaccg ctcggaattc |
| 6841 | cttttgcacg agggccggtt gctggatatc tcgatatcag ctgtcctgaa ctctctggtt |
| 6901 | gcatctgtca tctgtgatgt actgtttcag attgggcagt tatgacataa gtgagaaggc |
| 6961 | aagcatgtgt gagtgaagat tagagggtca catataactg gctgttttct gttcttgttt |
| 7021 | atccagcgta ggaagaagga aaagaaaatc tttgctcctc tgccccattc actatttacc |
| 7081 | aattgggaat taaagaaata attaatttga acagttatga aattaatatt tgctgtctgt |
| 7141 | gtgtataagt acatcctttg gggttttttt tttctctttt ttttaaccaa agttgctgtc |
| 7201 | tagtgcattc aaaggtcact ttttgttctt cacagatctt tttaatgttc tttcccatgt |
| 7261 | tgtattgcat ttttggggga agcaaattga ctttaaagaa aaaagttgtg gcaaaagatg |
| 7321 | ctaagatgcg aaaatttcac cacactgagt caaaaaggtg aaaaattatc catttcctat |
| 7381 | gcgttttact cctcagagaa tgaaaaaaac tgcatcccat cacccaaagt tctgtgcaat |
| 7441 | agaaatttct acagatacag gtataggggc tcaaggaggt atgtcggtca gtagtcaaaa |
| 7501 | ctatgaaatg atactggttt ctccacagga atatggttcc attaggctgg gagcaaaaac |
| 7561 | aatgtttttt aagattgaga atacatacct gacaacgatc cggaaactgc tcctcaccac |
| 7621 | tcccgtcatg cctgctgtcg gcgtttgacc ttccacgtga cagttcttca caattccttt |
| 7681 | catcattttt taaatatttt ttttactgcc tatgggctgt gatgtatata gaagttgtac |
| 7741 | attaaacata ccctcatttt tttcttttct tttttttttt tttttttagt acaaagtttt |
| 7801 | agtttctttt tcatgatgtg gtaactacga agtgatggta gatttaaata attttttatt |
| 7861 | tttattttat atattttttc attagggcca tatctccaaa aaaagaaaga aaaaatacaa |
| 7921 | aaaacaaaaa caaaaaaaaa agagggtaat gtacaagttt ctgtatgtat aaagtcatgc |
| 7981 | tcgatttcag gagagcagct gatcacaatt tgcttcatga atcaaggtgt ggaaatggtt |
| 8041 | atatatggat tgatttagaa aatggttacc agtacagtca aaaaagagaa aatgaaaaaa |
| 8101 | atacaactaa aaggaagaaa cacaacttca aagatttttc agtgatgaga atccacattt |
| 8161 | gtatttcaag ataatgtagt ttaaaaaaaa aaaaaagaaa aaaacttgat gtaaattcct |
| 8221 | ccttttcctc tggcttaatg aatatcattt attcagtata aaatctttat atgttccaca |
| 8281 | tgttaagaat aaatgtacat taaatcttgt taagcactgt gatgggtgtt cttgaatact |
| 8341 | gttctagttt ccttaaagtg gtttcctagt aatcaagtta tttacaagaa ataggggaat |
| 8401 | gcagcagtgt attcacatta taaaacccta catttggaag agacctttag gggttaccta |
| 8461 | ctttagagtg gggagcaaca gtttgatttt ctcaaattac ttagctaatt agtctttctt |
| 8521 | tgaagcaatt aactctaacg acattgaggt atgatcattt tcagtattta tgggaggtgg |
| 8581 | ctgctgaccc acttgaggtg agatctcaga agcttaactg gcctgaaaat gtaacattct |
| 8641 | gccttttact aactccatct tagtttaatc aaagttcaat ctattccttg tttcttctgt |
| 8701 | gtgcctcaga gttattttgc atttagttta ctccaccgtg tataatattt atactgtgca |
| 8761 | atgttaaaaa agaatctgtt atattgtatg tggtgtacat agtgcaaagt gatgatttct |
| 8821 | atttcagggc atattatggt tctcatattc cttcctacct ggtgcacagt agctttttaa |
| 8881 | tactagtcac ttctaattta aactttctct tcctgggtca ttgactgtta ctgtgtaata |
| 8941 | atcgatttct ttgaaactgc tgcataatta tgctgttagt ggacctctac ctcttctctt |
| 9001 | ccctctccca atcacagtat actcagaatc cccagcccct cgcatacatt gtgtcggttc |
| 9061 | acattactca cagtaatata tggaagagtt agacaagaac atgcagttac agtcattgtg |
| 9121 | agacgtgact ctccagtgtc acgaggaaaa aaatcatctt ttctgcaaac agtctctcat |
| 9181 | ctgtcaactc ccacattact gagtcaaaca gtcttcttac ataacaatgc aaccaaatat |
| 9241 | atgttgaatt aaagacccat ttataattct gctttaaata catctgcttg ctaagaacag |
| 9301 | atttcagtgc tccaagcttc aaatatggag atttgtaaga gggaattcaa tattattcta |
| 9361 | atttctctct tacagagtac aaataaaagg tgtatacaaa ctccgaacat atccagtatt |
| 9421 | ccaattcctt tgtcaatcag aagagtaaaa taattaacaa aagactgttg ttatggtttg |
| 9481 | cattgtaacc gatacgcaga gtctgaccgt tgggcaacaa gtttttctat cctgatgcgc |
| 9541 | aacacagtct ctagagacta atccaggaag actttagcct cctttccata ttctcacccc |
| 9601 | cgaatcaaga tttacagaag cccacgaaga atttacagcc tgcttgagat catcttgcct |
| 9661 | ataaactgag ttattgcttt gtcctaaaaa ttagtcggtt tttttttttc tatgaggctt |
| 9721 | ttcagaaatt tacaggatgc ccagacttta catgtgtacc aaaaaaaaaa aaaagataaa |
| 9781 | aaataaaggt gcaaagaaag tttagtattt tggaatggtg ctataaagtt gaa |

SEQ ID NO: 57 Human ARID1B Amino Acid Sequence isoform C
(NP_001333742.1)

| | |
|---|---|
| 1 | mahnagaaaa agthsaksgg seaalkeggs aaalssssss saaaaaasss sssgpgsame |
| 61 | tgllpnhklk tvgeapaapp hqqhhhhhha hhhhhhahhl hhhhalqqql nqfqqqqqqq |
| 121 | qqqqqqqqqq qhpisnnnsl ggagggapqp gpdmeqpqhg gakdsaaggq adppgpplls |
| 181 | kpgdeddapp kmgepaggry ehpglgalgt qqppvavpgg gggpaavpef nnyygsaapa |
| 241 | sggpggragp cfdqhggqqs pgmgmmhsas aaaagapgsm dplqnshegy pnsqcnhypg |
| 301 | ysrpgagggg gggggggggs ggggggggag aggagagava aaaaaaaaaa gggggggygg |
| 361 | ssagygvlss prqqgggmmm gpggggaasl skaaagsaag gfqrfagqnq hpsgatptln |
| 421 | qlltspspmm rsyggsypey sspsappppp sqpqsqaaaa gaaaggqqaa agmglgkdmg |
| 481 | aqyaaaspaw aaaqqrshpa mspgtpgptm grsqgspmdp mvmkrpqlyg mgsnphsqpq |
| 541 | qsspypggsy gppgpqrypi giqgrtpgam agmqypqqqm ppqygqqgvs gycqqqqqpy |
| 601 | ysqqpqpphl ppqaqylpsq sqqryqpqqd msqegygtrs qpplapgkpn hedlnliqqe |
| 661 | rpsslpdlsg siddlptgte atlssavsas gstssqgdqs npaqspfsph asphlssipg |
| 721 | gpspspvgsp vgsnqsrsgp ispasipgsq mppqppgsqs essshpalsq spmpqergfm |
| 781 | agtqrnpqma qygpqqtgps msphpspggq mhagissfqq snssgtygpq msqygpqgny |
| 841 | srppaysgvp sasysgpgpg mgisannqmh gqgpsqpcga vplgrmpsag mqnrpfpgnm |
| 901 | ssmtpsspgm sqqggpgmgp pmptvnrkaq eaaaavmqaa ansaqsrqgs fpgmnqsglm |
| 961 | assspysqpm nnssslmntq appysmapam vnssaasvgl admmspgesk lplplkadgk |
| 1021 | eegtpqpesk skdsyssqgi sqpptpgnlp vpspmspssa sissfhgdes dsisspgwpk |
| 1081 | tpsspkssss tttgekitkv yelgneperk lwvdryltfm eergspvssl pavgkkpldl |
| 1141 | frlyvcvkei gglaqvnknk kwrelatnln vgtsssaass lkkqyiqylf afeckierge |
| 1201 | epppevfstg dtkkqpklqp pspansgslq gpqtpqstgs nsmaevpgdl kpptpastph |
| 1261 | gqmtpmqggr sstisvhdpf sdvsdssfpk rnsmtpnapy qqgmsmpdvm grmpyepnkd |
| 1321 | pfggmrkvpg ssepfmtqgq mpnssmqdmy nqspsgamsn lgmgqrqqfp ygasydrrhe |
| 1381 | pygqqypgqg ppsgqppygg hqpglypqqp nykrhmdgmy gppakrhegd mynmqyssqq |
| 1441 | qemynqyggs ysgpdrrpiq gqypypysre rmqgpgqiqt hgippqmmgg plqssssegp |
| 1501 | qqnmwaarnd mpypyqnrqg pggptqappy pgmnrtddmm vpdqrinhes qwpshvsqrq |
| 1561 | pymsssasmq pitrppqpsy qtppslpnhi srapspasfq rslenrmsps kspflpsmkm |
| 1621 | qkvmptvpts qvtgpppqpp pirreitfpp gsveasqpvl kqrrkitskd ivtpeawrvm |
| 1681 | mslksgllae stwaldtini llyddstvat fnlsqlsgfl ellveyfrkc lidifgilme |
| 1741 | yevgdpsqka ldhnaarkdd sqsladdsgk eeedaecidd deedeedeee dsektesdek |
| 1801 | ssialtapda aadpkekpkq askfdklpik ivkknnlfvv drsdklgrvq efnsgllhwq |
| 1861 | lgggdttehi qthfeskmei pprrrppppl ssagrkkeqe gkgdseeqqe ksiiatiddv |
| 1921 | lsarpgalpe danpgpqtes skfpfgiqqa kshrniklle deprsrdetp lctiahwqds |
| 1981 | lakrcicvsn ivrslsfvpg ndaemskhpg lvlilgklil lhhehperkr apqtyekeed |
| 2041 | edkgvacskd ewwwdclevl rdntlvtlan isgqldlsay tesiclpild gllhwmvcps |
| 2101 | aeaqdpfptv gpnsvlspqr lvletlckls iqdnnvdlil atppfsrqek fyatlvryvg |
| 2161 | drknpvcrem smallsnlaq gdalaaraia vqkgsignli sfledgvtma qyqqsqhnlm |
| 2221 | hmqppplepp svdmmcraak allamarvde nrsefllheg rlldisisav lnslvasvic |
| 2281 | dvlfqigql |

SEQ ID NO: 58 Mouse ARID1B cDNA Sequence (NM_001085355.1, CDS: from 22
to 6756)

| | |
|---|---|
| 1 | tcggcgggcc ccggctcgac catggagacc gggctgctcc ccaaccacaa actgaaagcc |
| 61 | gttggcgagg cccccgctgc accgccccat cagcagcacc accaccacca tgcccaccac |
| 121 | caccaccacc accatgccca ccacctccac cacctccacc accaccacgc actacagcag |
| 181 | cagctaaacc agttccagca gccgcagccg ccgcagccac agcagcagca gccgccgcca |
| 241 | ccgccgcagc agcagcatcc cactgccaac aacagcctgg gcggtgcggg cggcggcgcg |
| 301 | cctcagcccg gcccggacat ggagcagccg caacatggag gcgccaagga cagtgtcgcg |
| 361 | ggcaatcagg ctgacccgca gggccagcct ctgctgagca aaccgggcga cgaggacgac |
| 421 | gcgccgccca agatggggga gccggcgggc agccgctatg agcacccggg cctgggcgcg |
| 481 | cagcagcagc ccgcgccggt cgccgtgccc gggggcggcg gcggcccagc ggccgtctcg |
| 541 | gagtttaata attactatgg cagcgctgcc cctgctagcg gcggccccgg cggccgcgct |
| 601 | gggccttgct ttgatcaaca tggcggacaa caaagccccg ggatggggat gatgcactcc |
| 661 | gcctctgccg ccgccggggc ccccagcagc atggaccccc tgcagaactc ccacgaaggg |
| 721 | taccccaaca gccagtacaa ccattatccg ggctacagcc ggcccggcgc gggcggcggc |
| 781 | ggcggcggcg gcggaggagg aggaggcagc ggaggaggtg gaggaggagg aggagcagga |
| 841 | ggagcaggag gagcagcggc agcggcagca ggagccggag ctgtggcggc ggcggccgcg |
| 901 | gcggcggcgg cagcagcagc agcagcagga ggaggcggtg gcggcggcta tgggagctcg |
| 961 | tcctcggggt acggggtgct gagctccccg cggcagcagg gcggcggcat gatgatgggc |
| 1021 | cccgggggcg gcggggccgc gagcctcagc aaggcggccg ccggcgcggc ggcggcggcg |
| 1081 | gggggcttcc agcgcttcgc cggccagaac cagcacccgt cgggggctac accgaccctc |
| 1141 | aaccagctgc tcacctcacc cagccccatg atgaggagct acggcggtag ctaccccgac |
| 1201 | tacagcagct ccagcgcgcc gccgccgccg tcgcagcccc agtcccaggc ggcggcgggg |
| 1261 | gcggcggcgg gtggccagca ggcggccgcg ggcatgggct tgggcaagga cctaggcgcc |
| 1321 | cagtacgccg ctgccagccc ggcctgggcg gccgcgcaac aaaggagtca cccggcgatg |
| 1381 | agccccggca cccccggacc gaccatgggc agatcccagg gcagcccgat ggacccaatg |
| 1441 | gtgatgaaga gacctcagtt gtatgggatg ggtactcacc cccactccca gccacagcag |
| 1501 | agcagcccat acccaggagg ctcctacggt cccccaggtg cacagcggta tccccttggc |
| 1561 | atgcagggcc gggctccagg ggccctggga ggcttgcagt acccgcagca gcagatgcca |
| 1621 | ccgcagtacg gacagcaagc tgtgagtggc tactgccagc aaggccagca gccatactac |
| 1681 | aaccagcagc cgcagccctc gcacctcccg ccccaggcac agtacctgca gccggcggcg |
| 1741 | gcgcagtccc agcagaggta ccagccacag caggacatgt ctcaagaagg ctatggaact |
| 1801 | agatctcagc ctcctctggc ccctggaaaa tccaaccatg aagacttgaa tttaattcaa |
| 1861 | caggaaagac catcgagtct accagacctg tctggctcca tcgatgacct ccccacggga |
| 1921 | acagaagcaa ctctgagctc agcagtcagt gcatccgggt ctacaagcag ccagggagat |
| 1981 | cagagcaacc cagcgcagtc tcctttctcc ccacatgcat cacctcacct ctccagcatc |
| 2041 | cctggagggc cgtcaccttc tcctgttggc tctcctgtgg gaagcaacca atcgaggtct |
| 2101 | ggtccgatct cccctgcgag tattccaggt agccagatgc ctccgcaacc acctggaagc |
| 2161 | cagtcagaat ccagttccca tcctgccttg agccagtcac caatgccaca ggaaagaggt |
| 2221 | tttatgacag gcactcagag aaaccctcag atgtctcagt acggacctca gcagacagga |
| 2281 | ccatccatgt cgcctcaccc atctcctggg ggccagatgc atcctgggat cagtaacttt |
| 2341 | cagcagagta actcaagtgg cacgtacggc ccacagatga gccagtatgg accccaaggc |
| 2401 | aactactcca gaaccccaac atatagcggg gtacccagtg caagctacag cggcccaggg |
| 2461 | cccggtatgg gcatcaatgc caacaaccag atgcatggac aagggccagc ccagccatgt |
| 2521 | ggtgctatgc ccctgggacg aatgccttca gctgggatgc agaacagacc atttcctgga |
| 2581 | accatgagca gcgtcacccc cagttctcct ggcatgtctc aacagggagg gccaggaatg |
| 2641 | ggcccaccaa tgcccactgt gaaccggaag gcccaggaag ctgccgcagc tgtgatgcag |
| 2701 | gctgctgcaa actcagcaca aagcaggcaa ggcagttttc ctggcatgaa ccagagtggc |
| 2761 | ctggtggcct ccagctctcc ctacagccag tccatgaaca acaactccag cctgatgagc |
| 2821 | acccaggccc agccctacag catgacgccc acaatggtga acagctccac agcatctatg |
| 2881 | ggtcttgcag atatgatgtc tcccagtgag tccaaattgt ctgtgcctct taaagcagat |
| 2941 | ggtaaagaag aaggcgtgtc ccagcctgag agcaagtcaa aggacagcta tggctctcag |
| 3001 | ggcatttccc agcctccaac cccaggcaac ctgcctgtcc cttccccaat gtctcccagc |
| 3061 | tctgccagca tctcctcctt tcatggagat gagagtgaca gcattagcag cccaggctgg |
| 3121 | cccaagacac catcaagccc taagtccagc tcttcctcca ccactgggga gaagatcacg |
| 3181 | aaggtctatg agctggggaa tgagccggag aggaagctgt gggtcgaccg ttacctaacg |
| 3241 | ttcatggaag agaggggctc cccggtgtcc agtctgccag cagtgggcaa gaagcccctg |
| 3301 | gacctgttcc gactgtatgt ctgcgtcaag gagattggag gtttggcgca ggttaataaa |
| 3361 | aacaagaagt ggcgtgagct ggcaaccaac ctgaacgttg gcacttccag cagcgcagcc |
| 3421 | agctctctga aaaagcagta tattcagtac ctgttcgcct ttgagtgcaa aactgagcgc |
| 3481 | ggggaggagc ccccacctga agtcttcagc accggggatt cgaagaagca gccaaagctc |
| 3541 | cagccgccat ctcctgctaa ctcaggatcc ttacaaggcc cacagactcc acagtcaact |
| 3601 | gggagcaatt cgatggcaga ggttccaggt gacctgaagc caccaacccc agcctctacc |
| 3661 | cctcatggac agatgactcc catgcaaagc ggaagaagca gtacagtcag tgtgcatgac |
| 3721 | ccgttctcag acgtgagtga ctcagcgtac ccaaaacgga actccatgac tccaaacgcc |
| 3781 | ccataccagc agggcatggg catgccagac atgatgggca ggatgcccta tgaacccaac |
| 3841 | aaggaccctt tcagtggaat gagaaaagtg cctggaagta gtgagccctt tatgacacaa |
| 3901 | ggacaggtgc ccaacagcgg catgcaggac atgtacaacc agagcccctc aggggccatg |
| 3961 | tccaatctgg gcatgggaca gcggcagcag tttccctatg gaaccagtta tgaccgaagg |
| 4021 | catgaggctt acggacagca gtacccaggc caaggccctc ccacaggaca gccaccgtat |
| 4081 | ggaggacacc agcctggcct gtacccacag cagccgaatt acaaacgtca tatggatggc |
| 4141 | atgtacgggc ctccagccaa gcggcacgag ggagacatgt acaacatgca gtatggcagc |
| 4201 | cagcagcagg agatgtataa ccagtatgga ggctcctact ctggcccgga cagaaggccc |
| 4261 | atccagggac aatatcccta cccctacaac agagaaagga tgcagggccc aggccagatg |
| 4321 | cagccacacg gaatcccacc tcagatgatg gggggcccca tgcagtcatc ctccagcgag |
| 4381 | gggcctcagc agaacatgtg ggctacacgc aacgatatgc cttatcccta ccagagcagg |
| 4441 | caaggcccgg gcggccctgc acaggccccc ccttacccag gcatgaaccg cacagatgat |
| 4501 | atgatggtac ctgagcagag gatcaatcac gagagccagt ggccttctca cgtcagccag |
| 4561 | cgccagcctt acatgtcatc ttcggcctcc atgcagccca tcacgcgccc acctcagtca |
| 4621 | tcctaccaga cgccgccgtc actgccaaac cacatctcca gggcacccag ccccgcctcc |
| 4681 | ttccagcgct ccctggagag tcgcatgtct ccaagcaagt ctcccttcct gcccaccatg |
| 4741 | aagatgcaga aggtcatgcc cacagtcccc acatcccagg tcaccgggcc ccccccacag |
| 4801 | cctccaccaa tcagaaggga gattaccttt cctcctggct ccgtagaagc atcacagcca |
| 4861 | atcctgaaac aaaggcgaaa gattacctca aaagatattg ttactcccga ggcgtggcgt |
| 4921 | gtgatgatgt cccttaaatc gggtctgttg gctgagagca cgtgggctct ggacaccatc |
| 4981 | aatattctcc tctatgatga cagcaccgtc gccaccttca atctttccca gctgtctgga |
| 5041 | ttcctggaac tattagtaga gtactttcga aaatgcctaa ttgacatttt cggaattctt |
| 5101 | atggaatatg aagtgggtga ccccagccaa aaggctcttg atcaccgttc agggaagaaa |
| 5161 | gatgacagcc agtccctgga agatgattct gggaaggaag acgatgatgc tgagtgtctt |
| 5221 | gtggaagagg aggaggagga agaggaggag gaggaagaca gtgaaaagat agagtcagag |
| 5281 | gggaagagca gccctgccct agctgctcca gatgcctccg tggaccccaa ggagacgcca |
| 5341 | aagcaggcca gtaagtttga caagctgccc ataaagattg tcaaaaagaa caagctgttt |
| 5401 | gtggtggacc ggtccgacaa gctgggccga gtgcaggagt tcagcagcgg gctcctccac |
| 5461 | tggcagctgg gtggtggcga cactaccgag cacatccaga ctcacttcga gagcaagatg |
| 5521 | gagatccctc ctcgcaggcg tccacctccg cctctaagct ccacgggtaa gaagaaagag |
| 5581 | ctggaaggca aaggtgattc tgaagagcag ccagagaaaa gtatcatagc caccatcgat |
| 5641 | gacgtcttgt ctgcccggcc aggggctctg cctgaagaca ccaacccagg accccagacc |
| 5701 | gacagcggca agtttccctt tggaatccag caggccaaaa gccaccggaa catcaggctc |
| 5761 | ctggaagacg agcccaggag ccgagacgag acgccgctgt gcaccatcgc gcactggcag |
| 5821 | gactcactgg ccaagcgctg catctgtgtg tcgaacatcg tgcggagctt gtctttcgtg |
| 5881 | cctggcaacg acgcagagat gtccaaacac ccgggcttgg tgctgatcct gggaaagctg |
| 5941 | attctgctgc atcacgagca tccggagaga aagcgggcgc cacagaccta tgagaaggag |
| 6001 | gaggacgagg acaagggggt ggcctgcagc aaagatgagt ggtggtggga ctgcctcgag |
| 6061 | gtcttgcggg ataacaccct ggtcacgttg gcgaacattt ccgggcagct agacttgtct |
| 6121 | gcttacacag agagcatctg cttgccgatc ctggacggct tgctacactg gatggtgtgc |
| 6181 | ccgtccgcag aggctcagga cccctttccc actgtggggc ccaactcagt cctgtcgccg |
| 6241 | cagagacttg tgctggagac cctgtgtaaa ctcagtatcc aggacaacaa cgtggacctg |
| 6301 | atcttggcca cgcctccatt tagtcgtcag gagaaatttt atgctacatt agttaggtac |
| 6361 | gttggggatc gcaaaaatcc agtctgtcga gaaatgtcca tggcgctttt atcgaacctt |
| 6421 | gcccaggggg acacactggc ggcgagggca atagctgtgc agaaaggaag cattggtaac |
| 6481 | ttgataagct tcctagagga cggggtgacg atggcgcagt accagcagag ccagcataac |
| 6541 | cttatgcaca tgcagccccc acctctggaa ccccctagtg tagacatgat gtgccgggcg |
| 6601 | gccaaagctc tgctggccat ggccagagtg gacgagaacc gctcggagtt ccttttgcac |
| 6661 | gagggtcggt tgctggatat ctcaatatca gctgtcctga actctctggt tgcatctgtc |
| 6721 | atctgtgatg tactgtttca gattgggcag ttatgacatc cgtgaaggca cacatgtgtg |
| 6781 | agtgaacatt agagggtcac atataactgg ctgttttctg ttctcgttta tccagtgtaa |
| 6841 | gaagaaggaa aagaaaaatc tttgctcctc tgccccgttt actatttacc aattgggaat |
| 6901 | taaatcatta atttgaacag ttataaaatt aatatttgct gtctgtgtgt ataagtacat |
| 6961 | cctctggcgg ttttctgttt cttttttttt taaccaaagt tgccgtctag tgcattcaaa |
| 7021 | ggtcacaatt tttgtttgtt tgtttgtttg tttgtttttt cataattttt ttcatgttgt |
| 7081 | attgcagtct ttgggaagtg aattgacttt ataaagaaaa acgttttggc aaaaagtgct |
| 7141 | aagatagaaa aatgtcacca cactgggtca aaaacgtgaa aggaaaaatt gattcttaaa |
| 7201 | ttgatttcct atgaatttta ttcttcacag aatgataaaa gctaaactgc accccgtcac |
| 7261 | ccaaagctct gtgcaataga aacttctaga gatatagtgt aggggctgaa ggaggtatgg |
| 7321 | cagcagtagt cagggtcaat gatactgctt tctccaccgg aaagtggtta cgttaggcct |
| 7381 | cgagcaaaaa acagcgctct cagataggtg caaaaatcca ctcctagcag ccaacagcag |
| 7441 | gatcgcttcc tcaccacgac cgccatgtct gctgtggctc agcctccacg ggacaaagct |
| 7501 | tcaagatttc tttcatcatt tttttaaata ttttttttac tgcctatggg ctgtgatgta |
| 7561 | tatagaagtt gtacattaaa cataccctca tttttttctt cttttctttt tttctttttt |
| 7621 | tctttttctt tttttttttt tttagtacaa agtttttagt ttctttttca tgatgtggta |
| 7681 | actacgaagt gatggtagat ttaaataatt ttttattttt attttatata ttttttcatt |
| 7741 | aggaccatat ctccaaaaaa caagaaaaag aaacaaaaaa tacaaaaaat aaaaacaaac |
| 7801 | aaaaaaagag ggtaatgtac aagtttctgt atgtataaag tcatgctctg ttgggagagc |
| 7861 | ggctgatccc agtttgcttc atgaatcaaa gtgtggaaat ggttgcatac agattgattt |
| 7921 | agaaaatgga caccagtaca tacaaaaaaa gaaaaaagaa agaaaaccaa ctaaatggaa |
| 7981 | gaaacacaac ttcaaagatt tttctgtgac aagaatccac atttgtattt caagataatg |
| 8041 | tagtttaaga aaagaaaaaa aagaaaaaaa aagaaaaaaa cttgatgtaa attcctcctt |
| 8101 | ttcctctggc ttaatgaata tcatttattc agtataaaat ctttatatgt cccacatgtt |
| 8161 | aagaataaat gtacattaaa tcttgttacg cactgtgatg ggtgttcttg aatgctgttc |
| 8221 | tagtttgcct agcatggttg ccatagtaac caagttattt acaggaaata gggaagatgt |
| 8281 | aacaactgct tcctggtaat gatgcccaaa ggccagaagg gactttcagg gtttcctact |
| 8341 | tgagagtggg agcaacaatt tgattttctc agattgttta gctaattagg tcttctttga |
| 8401 | agcaattaac tctggtgaca ttgagaagtg gtaattccct catggatggg tggtggctgc |
| 8461 | caacccactg tgacatgggg ccctgcaagc taactggcct gaaaccacga ccttctgcct |
| 8521 | ctcactactg atttaaccca agtctgcacc cgtcatgttt cttctgtgtg cctccaagtt |
| 8581 | actctgcgtt agtttgctcc agcgtgtata atatttatat tgtgcaatgt taaagagaac |
| 8641 | gtgtcatatt gtatgccgtg tgtatagtgc caagtgatga ttctgtttca gagcatacct |
| 8701 | tccttcctgc ccagtccctg gctctctaat accccaccct gatggaaagt gcttcttcct |
| 8761 | gggtaattga ctgttactgt gtaacgctca gtctcattga aacttacata accatgctgc |
| 8821 | tggtgcccct tcctacccta cctctctcag cactcttcag ttgacacttc ccacacctgt |
| 8881 | cactgtggcc caccttgctc acgctgacat ctggaagagt tagacaggag cacacactta |
| 8941 | caacactagg agatgttatt ctggtgtcac gagaaagaaa ttggtttttc ctgcaaacag |
| 9001 | tcccatcacc aagcagcccc cacatcaggt cagcaaaaag atctgtgttg aatcaaaact |
| 9061 | ccatttataa ttctactaga tgggaataca tctgcttaca aaggacagat tttagtgttc |
| 9121 | tgtgatgaaa atatggagag tgcaagagag agttcaatgg aatcctaatc ttgctcttgc |
| 9181 | agacaatgaa tgaaaggtat agacaggctc agttccctgt cagaagagtg gtctcaaaga |
| 9241 | caagtggctg tatagcagcc aggcccagaa cagcctcgca gcacacacta acaccaagcg |
| 9301 | ggtgtctgag ctctcctagg aagccttgtg cctgccctcc ctccattcac ccagatccga |
| 9361 | ctcctggaag cccacgaaag agtcaccctt tgcttcacat ttcctgacga taccgagttg |
| 9421 | ctgctctgtc ctaaaaatat tagttctttt ccagggcttt cagaaatttg caggatgccc |
| 9481 | atactctaaa tgtgtaccaa aaagagagag aaataaaggt gcgaagaaag tttagtattt |
| 9541 | tggaatggtg cgataaaatg gaatctgttg gtttttaatg taacataaga tactattggc |
| 9601 | tggcactggc taaaaaaaat atctaagtgt tggagttgga tgcacaatca acttttactt |
| 9661 | agctattcaa agagtactta tgttttccaa gttaaaacag acttgttttt gacaggggcc |
| 9721 | gtgggtggtc ttatacaatg ccagctccta actgcagctt ctgagaactg gatatcgttt |
| 9781 | gccctgagag ctgcccgtct ccaactatgt gctgctgctg ccctgtgtgc tcagcccaca |
| 9841 | aggatgtgga gactggatag acaacccctt gcttcttgct gggttgtgct gagttctttg |
| 9901 | cagtccagtc aagtgcccag agctaccagc ctacgtccct catgcatcca agagaaatga |
| 9961 | tcttgactat catgatcaaa acagctgtag taatatttct agtaaatatt tctgatgact |
| 10021 | ctgtgtaatc tcctacaaca ggacactatt cattaacttg acagagacat gtgggcatgt |
| 10081 | ggtcctgctt tagtttaaca gacaagtcaa ccagttctca ttacttagga agagtgaggc |
| 10141 | tatgtctgtt acaatcccaa tgtggtgctt gcccttatcc aaagacagtc cgggggccct |
| 10201 | gtctgcctga actatgtctc gctccctctt gggcttccca ctgggatgtg aaaagataac |
| 10261 | caatggctcc caggttccca gtgcccccca aaccagtaat caggtctggg actacagaac |
| 10321 | ccgcaaaatc atacacaggc tgtttcaaag ccagtactct ctttatactc ctgcttcctc |
| 10381 | cagcccccat ttcacacccc acccaaatca caaggtcctc tgaagtctca gaactccaaa |
| 10441 | ttaacgttgg gatttacgat gtgaatgctg aggagaaaat tgggagttgg tgggagatca |
| 10501 | ccaaattgtc aaaactatga aactcatctg tcttcccaaa tctgacctca gggacttggg |
| 10561 | gggttcactc tggcttctgc cacagtattt tctggggaac caaaggcctc gggaatagag |
| 10621 | aaacaggttg ccggatatcc tggaagtcta agccatactg accagtttgt cttgagtgtt |
| 10681 | ttctttgtga gcctggaact gtccccggac ccctttcttt taaacatggt tcaggacttt |
| 10741 | aaaaaaaagc actgtatttt ttttatgtaa gccaagatgc cctccctagc agagatagcg |
| 10801 | ttgaactgtc tctagttctg tagcctgaga gacttaaatc gtttaacttc agtgtctttg |
| 10861 | tccactctgt tgaactgcta aggattctat tgaatgtgtt ctttgcggct ttggaggagt |
| 10921 | tgctgggtgt gtaagtcctg catccctttg cctggtatgt gtatattatt cctttgcctg |
| 10981 | gctgtgtatc gttcttcagt gtaagtacac ccacactctg tattcctttg cctgctcccc |
| 11041 | gcccccccac acacacacat cctgcatagt tttaaaataa ggcctgagag actgtttcta |
| 11101 | tttcctgtca tagctggtga cttttaacag ttgaggcgaa tggcctgtca cttgcctggg |
| 11161 | ttcccgtcag gggtgatcca tggaactcct cagtggaaca gaatttagga cagaagatcc |
| 11221 | caccttcctt ccaggcctgg ggagaatcag actgtgagat aaaccatgat gctgcccaat |
| 11281 | cccactgccc caccttgctt ttaaaataaa gtgcctccta acgtc |

SEQ ID NO: 59 Mouse ARID1B Amino Acid Sequence (NP_001078824.1)

| | |
|---|---|
| 1 | metgllpnhk lkavgeapaa pphqqhhhhh ahhhhhhhah hlhhlhhhha lqqqlnqfqq |
| 61 | pqppqpqqqq pppppqqqhp tannslggag ggapqpgpdm eqpqhggakd svagnqadpq |
| 121 | gqpllskpgd eddappkmge pagsryehpg lgaqqqpapv avpgggggpa avsefnnyyg |
| 181 | saapasggpg gragpcfdqh ggqqspgmgm mhsasaaaga pssmdplqns hegypnsqyn |
| 241 | hypgysrpga gggggggggg ggsggggggg gaggaggaaa aaagagavaa aaaaaaaaaa |
| 301 | aagggggggy gssssgygvl ssprqqgggm mmgpggggaa slskaaagaa aaaggfqrfa |
| 361 | gqnqhpsgat ptlnqlltsp spmmrsyggs ypdyssssap pppsqpqsqa aagaaaggqq |
| 421 | aaagmglgkd lgaqyaaasp awaaaqqrsh pamspgtpgp tmgrsqgspm dpmvmkrpql |
| 481 | ygmgthphsq pqqsspypgg sygppgaqry plgmqgrapg algglqypqq qmppqygqqa |
| 541 | vsgycqqqqq pyynqqpqps hlppqaqylq paaaqsqqry qpqqdmsqeg ygtrsqppla |
| 601 | pgksnhedln liqqerpssl pdlsgsiddl ptgteatlss avsasgstss qgdqsnpaqs |
| 661 | pfsphasphl ssipggpsps pvgspvgsnq srsgpispas ipgsqmppqp pgsqsesssh |
| 721 | palsqspmpq ergfmtgtqr npqmsqygpq qtgpsmsphp spggqmhpgi snfqqsnssg |
| 781 | tygpqmsqyg pqgnysrtpt ysgvpsasys gpgpgmgina nnqmhgqgpa qpcgamplgr |
| 841 | mpsagmqnrp fpgtmssvtp sspgmsqqgg pgmgppmptv nrkaqeaaaa vmqaaansaq |
| 901 | srqgsfpgmn qsglvasssp ysqsmnnnss lmstqaqpys mtptmvnsst asmgladmms |
| 961 | psesklsvpl kadgkeegvs qpeskskdsy gsqgisqppt pgnlpvpspm spssasissf |
| 1021 | hgdesdsiss pgwpktpssp ksssssttge kitkvyelgn eperklwvdr yltfmeergs |
| 1081 | pvsslpavgk kpldlfrlyv cvkeigglaq vnknkkwrel atnlnvgtss saasslkkqy |
| 1141 | iqylfafeck tergeepppe vfstgdskkq pklqppspan sgslqgpqtp qstgsnsmae |
| 1201 | vpgdlkpptp astphgqmtp mqsgrsstvs vhdpfsdvsd saypkrnsmt pnapyqqgmg |
| 1261 | mpdmmgrmpy epnkdpfsgm rkvpgssepf mtqgqvpnsg mqdmynqsps gamsnlgmgq |
| 1321 | rqqfpygtsy drrheaygqq ypgqgpptgq ppygghqpgl ypqqpnykrh mdgmygppak |
| 1381 | rhegdmynmq ygsqqqemyn qyggsysgpd rrpiqgqypy pynrermqgp gqmqphgipp |
| 1441 | qmmggpmqss ssegpqqnmw atrndmpypy qsrqgpggpa qappypgmnr tddmmvpeqr |
| 1501 | inhesqwpsh vsqrqpymss sasmqpitrp pqssyqtpps lpnhisraps pasfqrsles |
| 1561 | rmspskspfl ptmkmqkvmp tvptsqvtgp ppqpppirre itfppgsvea sqpilkqrrk |
| 1621 | itskdivtpe awrvmmslks gllaestwal dtinillydd stvatfnlsq lsgflellve |
| 1681 | yfrkclidif gilmeyevgd psqkaldhrs gkkddsqsle ddsgkeddda eclveeeeee |
| 1741 | eeeeedseki esegksspal aapdasvdpk etpkqaskfd klpikivkkn klfvvdrsdk |
| 1801 | 1grvqefssg 1lhwqlgggd ttehiqthfe skmeipprrr pppplsstgk kkelegkgds |
| 1861 | eeqpeksiia tiddvlsarp galpedtnpg pqtdsgkfpf giqqakshrn irlledeprs |
| 1921 | rdetplctia hwqdslakrc icvsnivrsl sfvpgndaem skhpglvlil gklillhheh |
| 1981 | perkrapqty ekeededkgv acskdewwwd clevlrdntl vtlanisgql dlsaytesic |
| 2041 | 1pildgllhw mvcpsaeaqd pfptvgpnsv lspqrlvlet lcklsiqdnn vdlilatppf |
| 2101 | srqekfyatl vryvgdrknp vcremsmall snlaqgdtla araiavqkgs ignlisfled |
| 2161 | gvtmaqyqqs qhnlmhmqpp pleppsvdmm craakallam arvdenrsef llhegrlldi |
| 2221 | sisavlnslv asvicdvlfq igql |

SEQ ID NO: 60 Human SMARCC1 cDNA Sequence (NM_003074.3, CDS: 119-
3436)

| | |
|---|---|
| 1 | ctgggcgggg ccgggaagcg gcagtggcgg ctacgcgcgc gggggtgcgc gcgggaacga |
| 61 | ccgggaaaca ccgcgagggc cggggtgggc caggctgtgg ggacgacggg ctgcgacgat |
| 121 | ggccgcagcg gcgggcggcg gcgggccggg gacagcggta ggcgccacgg gctcggggat |
| 181 | tgcggcggca gccgcaggcc tagctgttta tcgacggaag gatgggggcc cggccaccaa |
| 241 | gttttgggag agcccggaga cggtgtccca gctggattcg gtgcgggtct ggctgggcaa |
| 301 | gcactacaag aagtatgttc atgcggatgc tcctaccaat aaaacactgg ctgggctggt |
| 361 | ggtgcagctt cttcagttcc aggaagatgc ctttgggaag catgtcacca acccggcctt |
| 421 | caccaaactc cctgcaaagt gtttcatgga tttcaaagct ggaggcgcct tatgtcacat |
| 481 | tcttggggct gcttacaagt ataaaaatga acagggatgg cggaggtttg acctacagaa |
| 541 | cccatctcga atggatcgta atgtggaaat gtttatgaac attgaaaaaa cattggtgca |
| 601 | gaacaattgt ttgaccagac ccaacatcta cctcattcca gacattgatc tgaagttggc |
| 661 | taacaaattg aaagatatca tcaaacgaca tcagggaaca tttacggatg agaagtcaaa |
| 721 | agcttcccac cacatttacc catattcttc ctcacaagac gatgaagaat ggttgagacc |
| 781 | ggtgatgaga aaagagaagc aagtgttagt gcattggggc ttttacccag acagctatga |
| 841 | tacttgggtc catagtaatg atgttgatgc tgaaattgaa gatccaccaa ttccagaaaa |
| 901 | accatggaag gttcatgtga aatggatttt ggacactgat attttcaatg aatggatgaa |
| 961 | tgaggaggat tatgaggtgg atgaaaatag gaagcctgtg agttttcgtc agcggatttc |
| 1021 | aaccaagaat gaagagccag tcagaagtcc agaaagaaga gatagaaaag catcagctaa |
| 1081 | tgctcgaaag aggaaacatt cgccttcgcc tccccctccg acaccaacag aatcacggaa |
| 1141 | gaagagtggg aagaaaggcc aagctagcct ttatgggaag cgcagaagtc agaaagagga |
| 1201 | agatgagcaa gaagatctaa ccaaggatat ggaagaccca acacctgtac ccaatataga |
| 1261 | agaagtagta cttcccaaaa atgtgaacct aaagaaagat agtgaaaata cacctgttaa |
| 1321 | aggaggaact gtagcggatc tagatgagca ggatgaagaa acagtcacag caggaggaaa |
| 1381 | ggaagatgaa gatcctgcca aaggtgatca gagtcgatca gttgaccttg gggaagataa |
| 1441 | tgtgacagag cagaccaatc acattattat tcctagttat gcatcatggt ttgattataa |
| 1501 | ctgtattcat gtgattgaac ggcgtgctct tcctgagttc ttcaatggaa aaaacaaatc |
| 1561 | caagactcca gaaatatact tggcatatcg aaattttatg attgacacgt atcgtctaaa |
| 1621 | cccccaagag tatttaacta gcactgcttg tcggaggaac ttgactggag atgtgtgtgc |
| 1681 | tgtgatgagg gtccatgcct ttttagagca gtggggactc gttaattacc aagttgaccc |
| 1741 | ggaaagtaga cccatggcaa tgggacctcc tcctactcct cattttaatg tattagctga |
| 1801 | taccccctct gggcttgtgc ctctgcatct tcgatcacct caggttcctg ctgctcaaca |
| 1861 | gatgctaaat tttcctgaga aaaacaagga aaaaccagtt gatttgcaga actttggtct |
| 1921 | ccgtactgac atttactcca agaaaacatt agcaaagagt aaaggtgcta gtgctggaag |
| 1981 | agaatggact gaacaggaga cccttctact cctggaggcc ctggagatgt acaaggatga |
| 2041 | ttggaacaaa gtgtcggaac atgttggaag tcgtactcag gatgaatgca tcctccactt |
| 2101 | tttgagactt cccattgagg acccatacct tgagaattca gatgcttccc ttgggccttt |
| 2161 | ggcctaccag cctgtcccct tcagtcagtc aggaaatcca gttatgagta ctgttgcttt |
| 2221 | tttggcatct gtggtggacc ctcgcgtggc atctgctgca gcaaaagcgg ctttggagga |
| 2281 | gttttctcgg gtccgggagg aggtaccact ggaattggtt gaagctcatg tcaagaaagt |
| 2341 | acaagaagca gcacgagcct ctgggaaagt ggatcccacc tacggtctgg agagcagctg |
| 2401 | cattgcaggc acagggcccg atgagccaga gaagcttgaa ggagctgaag aggaaaaaat |
| 2461 | ggaagccgac cctgatggtc agcagcctga aaaggcagaa aataaagtgg aaaatgaaac |
| 2521 | ggatgaaggt gataaagcac aagatggaga aaatgaaaaa aatagtgaaa aggaacagga |
| 2581 | tagtgaagtg agtgaggata ccaaatcaga agaaaaggag actgaagaga acaaagaact |
| 2641 | cactgataca tgtaaagaaa gagaaagtga tactgggaag aagaaagtag aacatgaaat |
| 2701 | ttccgaagga aatgttgcca cagccgcagc agctgctctt gcctcagcgg ctaccaaagc |
| 2761 | caagcacctg gctgcagtgg aagaaagaaa gatcaagtcc ctggtagctc tcttggttga |
| 2821 | gacacaaatg aagaaactag agatcaaact tcgacatttt gaagagctgg aaactatcat |
| 2881 | ggacagagag aaagaagctc tagaacaaca gaggcagcag ttgcttactg aacgccaaaa |
| 2941 | cttccacatg gaacagctga agtatgctga attacgagca cgacagcaaa tggaacagca |
| 3001 | gcagcatggc cagaaccctc aacaggcaca ccagcactca ggaggacctg gcctggcccc |
| 3061 | acttggagca gcagggcacc ctggcatgat gcctcatcaa cagccccctc cctaccctct |
| 3121 | gatgcaccac cagatgccac cacctcatcc accccagcca ggtcagatac caggcccagg |
| 3181 | ttccatgatg cccgggcagc acatgccagg ccgcatgatt cccactgttg cagccaacat |
| 3241 | ccacccctct gggagtggcc ctacccctcc tggcatgcca ccaatgccag gaaacatctt |
| 3301 | aggaccccgg gtacccctga cagcacctaa cggcatgtat ccccctccac cacagcagca |
| 3361 | gccaccgcca ccaccacctg cagatggggt ccctccgcct cctgctcctg gcccgccagc |
| 3421 | ctcagctgct ccttagcctg gaagatgcag ggaacctcca cgcccaccac catgagctgg |
| 3481 | agtggggatg acaagacttg tgttcctcaa ctttcttggg tttctttcag gatttttctt |
| 3541 | ctcacagctc caagcacgtg tcccgtgcct ccccactcct cttaccaccc ctctctctga |
| 3601 | cactttttgt gttgggtcct cagccaacac tcaaggggaa acctgtagtg acagtgtgcc |
| 3661 | ctggtcatcc ttaaaataac ctgcatctcc cctgtcctgg tgtgggagta agctgacagt |
| 3721 | ttctctgcag gtcctgtcaa ctttagcatg ctatgtcttt accatttttg ctctcttgca |
| 3781 | gttttttgct ttgtcttatg cttctatgga taatgctata taatcattat ctttttatct |
| 3841 | ttctgttatt attgttttaa aggagagcat cctaagttaa taggaaccaa aaaataatga |
| 3901 | tgggcagaag ggggggaata gccacagggg acaaacctta aggcattata agtgacctta |
| 3961 | tttctgcttt tctgagctaa gaatggtgct gatggtaaag tttgagactt ttgccacaca |
| 4021 | caaatttgtg aaaattaaac gagatgtgga aggagaacct cagtgatttt attccctagt |
| 4081 | gaggcctctg agggcctcca cactgcctgg cagaacatac cactgaacta gtatgtgcta |
| 4141 | gaggagggca caaacatccg ctccttccct aggcctgctg gctctggttt tctatgcaga |
| 4201 | tgattcattg gattgggggt gagtgttttg tttttctggg ggcagtgtga gctttgaggg |
| 4261 | ttggaatatt gggaggcatt ccttagtttc ctcaactagc ctggaaagtt aggagtctag |
| 4321 | ggtaattacc cccaatgagt ctagcctact attcactgct ttgtgtgcat ttttttctcc |
| 4381 | ctctttaaaa aaccctttaa aagaaaaaaa aaagtagata gtgctaaata ttttagctca |
| 4441 | tgaaacttgg ttaggatggc tgggggtaca agtccccaaa ctacctcttg ttacagtagc |
| 4501 | cagggagtgg aatttcgtca accggtactt ttaaggttag gatgggacgg gaaaagtgaa |
| 4561 | gcaggatatt agctccttat accttctccc ttccatttct gagatctcac attccatcta |
| 4621 | tcacagggtt ttcaaagaga tgctgagggt aacaaggaac tcacttggca gtcagagcat |
| 4681 | catgctttga ggtttggggt gctcaggctg ggagggtaga atgccattcc agaggacaag |
| 4741 | ccacaaaaat gccttaattt gagctcgtat ttacccctgc tgataagtga cttgagagtt |
| 4801 | cccggttttt tcctcttgtc cttccctccc ttctgtcctt ccatgtgtgg ggaaagggtg |
| 4861 | tttttggtag agcttggttt ccaaagcgcc tggctttctc acttcacatt ctcaagtggc |
| 4921 | agtttcatta tttagaatgc aaggtggaca tcttttggat atctttttct atatattttc |
| 4981 | taaagcttta catatgagag ggtataggga ggtgtttata aaacacttga gaactttttt |
| 5041 | ccttaatatc agaaagcaaa aaaataaaac cacaattgag atttgccttt caaaccctca |
| 5101 | ggtttgcctc taaccaggtg tccctggtca ccatcagagt actggaatac gggaaccgag |
| 5161 | gagaccttgg tccttttgtt tttgttctgg actcttggga gtggaaatga gaatgagttt |
| 5221 | attcctactg gagcttagtt ccaatgcatt tggctccaga aagaccccag tgccttttga |
| 5281 | caatggccag ggttttacct acttcctgcc agtctttccc aaaggaaact cattccaaat |
| 5341 | acttcttttt tcccctggag tccgagaagg aaaatggaat tctggttcat actgtggtcc |
| 5401 | cttgtaacct caggtcttta atgtgatcac tttcaaattt aaaagatcca ggtggaaata |
| 5461 | tttttactat agtaataatt ctacaaaata cctgaattct taacactgtt atatttcagt |
| 5521 | ataagtggtg gctttttctt ttcatgtctt tgatctggtt ttattcctgt aattcagcca |
| 5581 | cctgattttg tgaggggggg gaataatatg tggtttttgt acaaacatgt ttctcagtgt |
| 5641 | gttgttattt tggaaaaaat gaggggaggg agtttggcaa gaatggagaa aatgaatgaa |
| 5701 | gaaggcctaa tctctctctt tttcagtgaa taaatggaac accatttctg gattctaaaa |
| 5761 | aaaaaaaaaa aaaaaaaaaa |

SEQ ID NO: 61 Human SMARCC1 Amino Acid Sequence (NP_003065.3)

| | |
|---|---|
| 1 | maaaaggggp gtavgatgsg iaaaaaglav yrrkdggpat kfwespetvs qldsvrvwlg |
| 61 | khykkyvhad aptnktlagl vvqllqfqed afgkhvtnpa ftklpakcfm dfkaggalch |
| 121 | ilgaaykykn eqgwrrfdlq npsrmdrnve mfmniektlv qnncltrpni ylipdidlkl |
| 181 | anklkdiikr hqgtftdeks kashhiypys ssqddeewlr pvmrkekqvl vhwgfypdsy |
| 241 | dtwvhsndvd aeiedppipe kpwkvhvkwi ldtdifnewm needyevden rkpvsfrqri |
| 301 | stkneepvrs perrdrkasa narkrkhsps pppptptesr kksgkkgqas lygkrrsqke |
| 361 | edeqedltkd medptpvpni eevvlpknvn 1kkdsentpv kggtvadlde qdeetvtagg |
| 421 | kededpakgd qsrsvdlged nvteqtnhii ipsyaswfdy ncihvierra lpeffngknk |
| 481 | sktpeiylay rnfmidtyrl npqeyltsta crrnltgdvc avmrvhafle qwglvnyqvd |
| 541 | pesrpmamgp pptphfnvla dtpsglvplh lrspqvpaaq qmlnfpeknk ekpvdlqnfg |
| 601 | lrtdiyskkt lakskgasag rewteqetll llealemykd dwnkvsehvg srtqdecilh |
| 661 | flrlpiedpy lensdaslgp layqpvpfsq sgnpvmstva flasvvdprv asaaakaale |
| 721 | efsrvreevp lelveahvkk vqeaarasgk vdptygless ciagtgpdep eklegaeeek |
| 781 | meadpdgqqp ekaenkvene tdegdkaqdg eneknsekeq dsevsedtks eeketeenke |
| 841 | ltdtckeres dtgkkkvehe isegnvataa aaalasaatk akhlaaveer kikslvallv |
| 901 | etqmkkleik lrhfeeleti mdrekealeq qrqqllterq nfhmeqlkya elrarqqmeq |
| 961 | qqhgqnpqqa hqhsggpgla plgaaghpgm mphqqpppyp 1mhhqmppph ppqpgqipgp |
| 1021 | gsmmpgqhmp grmiptvaan ihpsgsgptp pgmppmpgni lgprvpltap ngmyppppqq |
| 1081 | qppppppadg vppppapgpp asap |

SEQ ID NO: 62 Mouse SMARCC1 cDNA Sequence (NM_009211.2, CDS: 94-3408)

| | |
|---|---|
| 1 | ggaggtggca tctgcgcgcg cgcgcgcggg tgcgaacggg aaacgccgcg agggccaggc |
| 61 | taggccgggc ggtagacacg acggacggtg actatggccg cgacagcggg tggcggtccg |
| 121 | ggagcagcag caggcgccgt gggtgcaggg ggtgcggcgg cggcctccgg gctggccgtg |
| 181 | taccggagga aggacggggg cccggccagc aagttttggg agagcccgga cacggtgtcc |
| 241 | cagctagatt cggtgcgagt ctggctgggc aagcactaca agaagtatgt tcatgcagat |
| 301 | gctcctacca ataaaacact agctggactg gtggtgcagc ttctacagtt ccaagaagat |
| 361 | gcctttggga agcatgtcac caacccagct ttcaccaaac tacctgcaaa atgtttcatg |
| 421 | gatttcaaag ctggaggcac cttgtgtcac attcttgggg cagcttacaa gtacaaaaat |
| 481 | gaacagggct ggcggagatt tgatcttcag aacccatccc gaatggatcg taacgttgaa |
| 541 | atgttcatga acattgagaa aacattggta cagaacaact gtctgactag accaaacatc |
| 601 | tacctcattc cagacattga tttgaagttg gctaacaagt tgaaagatat catcaaacgg |
| 661 | catcagggga catttactga tgagaagtca aaagcttccc accatattta tccatatcct |
| 721 | tcctcacaag aggatgagga gtggctgaga ccagtgatga ggagagacaa gcaggtgctg |
| 781 | gtgcactggg gtttctaccc agacagctat gacacttggg tccacagtaa tgatgttgat |
| 841 | gctgaaattg aagatgcacc aatcccagaa aagccctgga aggttcatgt aaaatggatt |
| 901 | ttggacactg acgttttcaa tgaatggatg aatgaagagg attatgaagt ggatgagaac |
| 961 | agaaagccag tgagctttcg tcaacgaatt tcaacaaaga atgaagagcc agtcagaagt |
| 1021 | ccagaaagga gagacagaaa agcctctgcc aactctagga agaggaaacc ttccccttct |
| 1081 | cctcctcctc ccacagccac agagtcccgc aagaagagcg ggaagaaagg acaagctagc |
| 1141 | ctttatggga aacgtagaag tcagaaggaa gaagatgagc aagaagatct taccaaggac |
| 1201 | atggaagacc ccacacctgt acctaacata gaggaagtgg ttctccctaa gaatgtaaac |
| 1261 | ccaaagaagg acagtgaaaa cacacccgtt aaaggaggca cggtggcaga tctagatgag |
| 1321 | caggatgaag aagcagttac aacaggagga aaggaagatg aagatcccag caaaggtgat |
| 1381 | ccaagtcgct cagttgaccc aggtgaagac aacgtgacag aacagaccaa tcacatcatt |
| 1441 | attcccagct acgcatcctg gtttgattat aattgtattc atgtcattga acggcgtgcg |
| 1501 | cttcctgagt tctttaatgg aaaaaacaaa tccaagaccc ctgaaatata cttggcatat |
| 1561 | cgaaatttta tgattgacac ataccgtcta aaccctcaag aatatttaac cagcactgct |
| 1621 | tgccggcgaa acctgactgg agatgtgtgt gctgtgatga gggttcatgc cttcttagag |
| 1681 | cagtggggtc ttgttaacta ccaagttgac ccagagagtc gacccatggc aatgggacct |
| 1741 | cctcccactc ctcacttcaa tgtgttagct gacacaccct ctgggcttgt gcccctgcat |
| 1801 | cttcgatcac ctcaggtccc tgccgctcaa cagatgttaa attttcctga gaagaacaag |
| 1861 | gaaaaaccaa ttgatttgca gaactttggt cttcgaactg acatttactc caagaaaaca |
| 1921 | ctggcaaaga gtaaaggtgc tagtgctgga agggagtgga cagaacagga gacccttctt |
| 1981 | ctcctagagg ctctggagat gtacaaggac gattggaata aagtgtcaga acatgttgga |
| 2041 | agccgtactc aggacgaatg catcctccac tttctgaggc ttcccattga ggacccttac |
| 2101 | cttgaaaatt cagatgcttc tcttgggcca ctggcttacc agcctgtccc tttcagccag |
| 2161 | tcgggaaacc cggtgatgag cactgttgcc tttttagcat ctgtcgttga cccccgtgta |
| 2221 | gcatctgctg cagcaaaagc agcgttggag gagttttctc gtgtccgaga agaagtaccc |
| 2281 | ctggaattgg ttgaagcaca tgtcaagaaa gtacaggaag ctgcaagagc ctctgggaag |
| 2341 | gtggacccca cctatggctt ggagagcagc tgtattgctg gcacagggcc tgacgagcca |
| 2401 | gagaagcttg aaggatctga agaagagaag atggaaacag atcctgatgg tcagcagcct |
| 2461 | gaaaaggcag aaaacaaagt ggaaaatgaa tcggatgaag gtgataaaat acaagatcga |
| 2521 | gagaatgaaa aaaacactga gaaggaacaa gatagtgacg tcagtgagga tgtcaagcca |
| 2581 | gaagaaaagg agaatgaaga gaacaaagag ctcactgata catgtaaaga aagagaaagc |
| 2641 | gatgccggga agaagaaagt ggaacacgag atttcggaag gaaacgttgc cacagccgca |
| 2701 | gcagctgctc tggcctcagc tgctactaaa gccaagcacc tggcggctgt tgaagaaaga |
| 2761 | aaaatcaagt ccttggtagc tctcttggtt gaaacacaaa tgaagaaact agagatcaaa |
| 2821 | cttcgacatt ttgaagagct ggagactata atggacagag agaaagaggc tctagaacaa |
| 2881 | cagagacagc agttgcttac tgagcgtcag aacttccaca tggaacagtt gaaatatgct |
| 2941 | gaactacgtg cccggcagca aatggagcag cagcagcagc atggccagac acctcagcag |
| 3001 | gcgcaccagc acacgggagg gccggggatg gccccacttg gagccacagg ccaccctggc |
| 3061 | atgatgccgc atcagcagcc ccctccctac ccactgatgc accatcagat gccgccaccc |
| 3121 | catcctcccc aaccaggtca aataccaggc cctggctcca tgatgcctgg ccagcccatg |
| 3181 | ccaggtcgca tgatccccgc tgtggcagcc aacattcacc ctactgggag tggccctacc |
| 3241 | cctcctggta tgcctccaat gcccggaaac atcttaggac cccgggtacc cctcacagca |
| 3301 | ccaaacggca tgtatcctcc tccaccacag cagcagcagc cgcctcctcc tgcagatggg |
| 3361 | gtccctccac ctcctgctcc aggcccaccc gcctcggcca ctccctagcc tggaagatac |
| 3421 | aagagcctcc acagccacca caagcaggaa tggggatggc aggacttgtg tctcggcttc |
| 3481 | cttggttttc ttgcaggatt tttttttcac aaccccaagc acaagcccca tgtctctcca |
| 3541 | ctccttgata cttcttgtgt caggtcctta gttgacactc attgggaagc ctgtggtgac |
| 3601 | tgatgtgctc tggtcattta aaaagtacca tgtgtctccc ctgtccccgt gtgacagatg |
| 3661 | ttggcaggtg gtctgcaggt cctgttgtgt tgacattagt attctttgtg tgtatctctc |
| 3721 | tctgtctctc tctctctgct ttgtctaagg cttcaatgta taatcctcta taattattgt |
| 3781 | cctttcttcc tttgtaatgg ttgttttttt aaggaaagta tcctaagtta atagaaacca |
| 3841 | aaaaaaatgg taatgggcag aaagagatag ccacagaggg acacacctta aggcattata |
| 3901 | agtgacctta tttctgctta tctgagctag agtggtgcta ctgatagagt ccctgagact |
| 3961 | tgtcacacat aagtgcacca agatgagaag agctggggaa agggggtatc ctttcgattt |
| 4021 | gatttcctgg tgaggaccat gaaggacttc cctgtgcctg gaagaacatg ccactgtacc |
| 4081 | tagtacacga tagatagcaa agagcacagc tttacaacaa gcccttccta ccttctcccg |
| 4141 | ccattctggt tgtctgtgca gaagatttgc aggattggaa catggtggtt gttttcccaa |
| 4201 | gggcagcgtg agctttcaga gttggggttt tcccagtcta acaaagataa agggtctggg |
| 4261 | gccctaccta caaaccttta ggaacccttc caaacctccc aaccttcccc aaacacatag |
| 4321 | ggcctaccct cgccacccca ataaacatta catgtttttt aaaccttcct ataagaaagg |
| 4381 | aaaaaaatgt aaaatgggtt atagattatg ttgaacattt tatctcatgc ggcttggtgg |
| 4441 | gggtgggggt acagatccct aaactacctc ttgctgtagc cagggtgagc ggggttctta |
| 4501 | agcggtactg aggtgcagaa cgggagtggg aatgctcaca tgtgatgagc agcctcctgt |
| 4561 | acctcacatt ctgagacctc acattccatc tgttgtcaca gggttatgga gactgtgcta |
| 4621 | atggcacaag gacctcactt ggctccagag tgcgaggctg taaggtttaa gtgccatccc |
| 4681 | agaggaattg ccaccaaaaa aaaaaaaaaa agccttaatc tgagcctgta tctacccctg |
| 4741 | ctgatgaaca actagatggg ttttggtttt gccagcttct ttcctccctc cctccctccc |
| 4801 | tccctccctc cctccctcct ttctgtcttt ccattagtag caaaagggtg tttttagcag |
| 4861 | aactttaagt ggcagtttca ttcttgagag tgcaaggtag agcaccttac gggtgtattt |
| 4921 | ttatgtgtat tttaaagctt tatgtatgag agctataggt aggcatttct taataacaca |
| 4981 | aaaacctaca gttgagattt gcctttaaga ctcttggttt tcctctaacc aggagcccac |
| 5041 | gtcaccgcca gagtcctgga gctagagcta atgactccag agccttgggg tggaaatgga |
| 5101 | gattcgctta ttccctgggt gcttgttttt cctccaggaa aaccccggtg tcttctgacc |
| 5161 | gcagccaggg ttgccctcct tccctccatt ctctcccaaa gtaaattgac tccagcactt |
| 5221 | gccttctccc cggagtccta ggggaggtat aggactctgc ttgtctgtaa cctgaggtct |
| 5281 | gtaatgtgat tgctttccag ttttgagaga tgcaagtggg aatagttttt acattgttga |
| 5341 | taatctatag aacctaagtt caacacttca acacagctct ttccatgact gtcagttagg |
| 5401 | tatcattcct gtaataacac ccatccagtt ttgtgagggg cgggcttgga tactgtgtgg |
| 5461 | tttttgtaca aatgtgtttc tcagtgtggg tttttgtttt ttgttgggtt tttttttttt |
| 5521 | ttttggtgtt tttttgtttg tttatttgtt ttttttcttt aggttttgtt ctaatgaggt |
| 5581 | aaaggagctt tgagagtttg ggagaaaatg aatgaaagtg gcttaatgtc cctcgtttgc |
| 5641 | attgaataaa tgaaatacca tttatgaatt ctaaaaaaaa aaaa |

SEQ ID NO: 63 Mouse SMARCC1 Amino Acid Sequence (NP_033237.2)

| | |
|---|---|
| 1 | maatagggpg aaagavgagg aaaasglavy rrkdggpask fwespdtvsq ldsvrvwlgk |
| 61 | hykkyvhada ptnktlaglv vqllqfqeda fgkhvtnpaf tklpakcfmd fkaggtlchi |
| 121 | lgaaykykne qgwrrfdlqn psrmdrnvem fmniektlvq nncltrpniy lipdidlkla |
| 181 | nklkdiikrh qgtftdeksk ashhiypyps sqedeewlrp vmrrdkqvlv hwgfypdsyd |
| 241 | twvhsndvda eiedapipek pwkvhvkwil dtdvfnewmn eedyevdenr kpvsfrqris |
| 301 | tkneepvrsp errdrkasan srkrkpspsp ppptatesrk ksgkkgqasl ygkrrsqkee |
| 361 | deqedltkdm edptpvpnie evvlpknvnp kkdsentpvk ggtvadldeq deeavttggk |
| 421 | ededpskgdp srsvdpgedn vteqtnhiii psyaswfdyn cihvierral peffngknks |
| 481 | ktpeiylayr nfmidtyrln pqeyltstac rrnltgdvca vmrvhafleq wglvnyqvdp |
| 541 | esrpmamgpp ptphfnvlad tpsglvplhl rspqvpaaqq mlnfpeknke kpidlqnfgl |
| 601 | rtdiyskktl akskgasagr ewteqetlll lealemykdd wnkvsehvgs rtqdecilhf |
| 661 | lrlpiedpyl ensdaslgpl ayqpvpfsqs gnpvmstvaf lasvvdprva saaakaalee |
| 721 | fsrvreevpl elveahvkkv qeaarasgkv dptyglessc iagtgpdepe klegseeekm |
| 781 | etdpdgqqpe kaenkvenes degdkiqdre nekntekeqd sdvsedvkpe ekeneenkel |
| 841 | tdtckeresd agkkkvehei segnvataaa aalasaatka khlaaveerk ikslvallve |
| 901 | tqmkkleikl rhfeeletim drekealeqq rqqllterqn fhmeqlkyae lrarqqmeqq |
| 961 | qqhgqtpqqa hqhtggpgma plgatghpgm mphqqpppyp 1mhhqmppph ppqpgqipgp |
| 1021 | gsmmpgqpmp grmipavaan ihptgsgptp pgmppmpgni lgprvpltap ngmyppppqq |
| 1081 | qqppppadgv ppppapgppa satp |

SEQ ID NO: 64 Human SMARCC2 cDNA Sequence Variant 1 (NM_003075.4,
CDS: 114-3758)

| | |
|---|---|
| 1 | ggaggcggcg gccgcggcgg cgggaggcgg cgggaggcgg gcggaggagg aggcggagga |
| 61 | ggcgggagct gagctgagtg gggcgggcgg cggcggggcc cgagccggag aagatggcgg |
| 121 | tgcggaagaa ggacggcggc cccaacgtga agtactacga ggccgcggac accgtgaccc |
| 181 | agttcgacaa cgtgcggctg tggctcggca agaactacaa gaagtatata caagctgaac |
| 241 | cacccaccaa caagtccctg tctagcctgg ttgtacagtt gctacaattt caggaagaag |
| 301 | tttttggcaa acatgtcagc aatgcaccgc tcactaaact gccgatcaaa tgtttcctag |
| 361 | atttcaaagc gggaggctcc ttgtgccaca ttcttgcagc tgcctacaaa ttcaagagtg |
| 421 | accagggatg gcggcgttac gatttccaga atccatcacg catggaccgc aatgtggaaa |
| 481 | tgtttatgac cattgagaag tccttggtgc agaataattg cctgtctcga cctaacattt |
| 541 | ttctgtgccc agaaattgag cccaaactac tagggaaatt aaaggacatt atcaagagac |
| 601 | accagggaac agtcactgag gataagaaca atgcctccca tgttgtgtat cctgtcccgg |
| 661 | ggaatctaga agaagaggaa tgggtacgac cagtcatgaa gagggataag caggttcttc |
| 721 | tgcactgggg ctactatcct gacagttacg acacgtggat cccagcgagt gaaattgagg |
| 781 | catctgtgga agatgctcca actcctgaga aacctaggaa ggttcatgca aagtggatcc |
| 841 | tggacaccga caccttcaat gaatggatga atgaggaaga ctatgaagta aatgatgaca |
| 901 | aaaaccctgt ctcccgccga aagaagattt cagccaagac actgacagat gaggtgaaca |
| 961 | gcccagattc agatcgacgg gacaagaagg ggggaaacta taagaagagg aagcgctccc |
| 1021 | cctctccttc accaacccca gaagcaaaga agaaaaatgc taagaaaggt ccctcaacac |
| 1081 | cttacactaa gtcaaagcgt ggccacagag aagaggagca agaagacctg acaaaggaca |
| 1141 | tggacgagcc ctcaccagtc cccaatgtag aagaggtgac acttcccaaa acagtcaaca |
| 1201 | caaagaaaga ctcagagtcg gccccagtca aaggcggcac catgaccgac ctggatgaac |
| 1261 | aggaagatga aagcatggag acgacgggca aggatgagga tgagaacagt acggggaaca |
| 1321 | agggagagca gaccaagaat ccagacctgc atgaggacaa tgtgactgaa cagacccacc |
| 1381 | acatcatcat tcccagctac gctgcctggt ttgactacaa tagtgttcat gccattgagc |
| 1441 | ggagggctct ccccgagttc ttcaacggca agaacaagtc caagactcca gagatctacc |
| 1501 | tggcctatcg aaactttatg attgacactt accgactgaa cccccaagag tatcttacct |
| 1561 | ctaccgcctg ccgccgaaac ctagcgggtg atgtctgtgc catcatgagg gtccatgcct |
| 1621 | tcctagaaca gtggggtctt attaactacc aggtggatgc tgagagtcga ccaaccccaa |
| 1681 | tggggcctcc gcctacctct cacttccatg tcttggctga cacaccatca gggctggtgc |
| 1741 | ctctgcagcc caagacacct cagcagacct ctgcttccca acaaatgctc aactttcctg |
| 1801 | acaaaggcaa agagaaacca acagacatgc aaaactttgg gctgcgcaca gacatgtaca |
| 1861 | caaaaaagaa tgttccctcc aagagcaagg ctgcagccag tgccactcgt gagtggacag |
| 1921 | aacaggaaac cctgcttctc ctggaggcac tggaaatgta caaagatgac tggaacaaag |
| 1981 | tgtccgagca tgtgggaagc cgcacacagg acgagtgcat cttgcatttt cttcgtcttc |
| 2041 | ccattgaaga cccatacctg gaggactcag aggcctccct aggccccctg gcctaccaac |
| 2101 | ccatcccctt cagtcagtcg ggcaaccctg ttatgagcac tgttgccttc ctggcctctg |
| 2161 | tcgtcgatcc ccgagtcgcc tctgctgctg caaagtcagc cctagaggag ttctccaaaa |
| 2221 | tgaaggaaga ggtacccacg gccttggtgg aggcccatgt tcgaaaagtg gaagaagcag |
| 2281 | ccaaagtaac aggcaaggcg gaccctgcct tcggtctgga aagcagtggc attgcaggaa |
| 2341 | ccacctctga tgagcctgag cggattgagg agagcgggaa tgacgaggct cgggtggaag |
| 2401 | gccaggccac agatgagaag aaggagccca aggaaccccg agaaggaggg ggtgctatag |
| 2461 | aggaggaagc aaaagagaaa accagcgagg ctcccaagaa ggatgaggag aaagggaaag |
| 2521 | aaggcgacag tgagaaggag tccgagaaga gtgatggaga cccaatagtc gatcctgaga |
| 2581 | aggagaagga gccaaaggaa gggcaggagg aagtgctgaa ggaagtggtg gagtctgagg |
| 2641 | gggaaaggaa gacaaaggtg gagcgggaca ttggcgaggg caacctctcc accgctgctg |
| 2701 | ccgccgccct ggccgccgcc gcagtgaaag ctaagcactt ggctgctgtt gaggaaagga |
| 2761 | agatcaaatc tttggtggcc ctgctggtgg agacccagat gaaaaagttg gagatcaaac |
| 2821 | ttcggcactt tgaggagctg gagactatca tggaccggga gcgagaagca ctggagtatc |
| 2881 | agaggcagca gctcctggcc gacagacaag ccttccacat ggagcagctg aagtatgcgg |
| 2941 | agatgagggc tcggcagcag cacttccaac agatgcacca acagcagcag cagccaccac |
| 3001 | cagccctgcc cccaggctcc cagcctatcc ccccaacagg ggctgctggg ccacccgcag |
| 3061 | tccatggctt ggctgtggct ccagcctctg tagtccctgc tcctgctggc agtggggccc |
| 3121 | ctccaggaag tttgggccct tctgaacaga ttgggcaggc agggtcaact gcagggccac |
| 3181 | agcagcagca accagctgga gccccccagc ctggggcagt cccaccaggg gttccccccc |
| 3241 | ctggacccca tggcccctca ccgttcccca accaacaaac tcctccctca atgatgccag |
| 3301 | gggcagtgcc aggcagcggg cacccaggcg tggcgggtaa tgctcctttg ggtttgcctt |
| 3361 | ttggcatgcc gcctcctcct cctcctcctg ctccatccat catcccattt ggtagtctag |
| 3421 | ctgactccat cagtattaac ctccccgctc ctcctaacct gcatgggcat caccaccatc |
| 3481 | tcccgttcgc cccgggcact ctccccccac ctaacctgcc tgtgtccatg gcgaaccctc |
| 3541 | tacatcctaa cctgccggcg accaccacca tgccatcttc cttgcctctc gggccggggc |
| 3601 | tcggatccgc cgcagcccaa agccctgcca ttgtggcagc tgttcagggc aacctcctgc |
| 3661 | ccagtgccag cccactgcca gacccaggca cccccctgcc tccagacccc acagccccga |
| 3721 | gcccaggcac ggtcacccct gtgccacctc cacagtgagg agccagccag acatctctcc |
| 3781 | ccctcacccc ctgtggacat cacggttcca ggaacagccc ttcccccacc actgggaccc |
| 3841 | tccccagcct ggagagttca tcactacgta aggaaagctc cttccgcccc tccaaagccc |
| 3901 | tcaccatgcc taacagaggc atgcattttt atatcagatt attcaaggac ttctgtttaa |
| 3961 | aagatgttta taatgtctgg gagagaggat aggatgggaa tgctgcccta aaggaagggc |
| 4021 | tggtgaaagg tgtttataca aggttctatt aaccacttct aagggtacac ctccctccaa |
| 4081 | actactgcat tttctatgga ttaaaaaaaa aaaaaaaaag tagattttaa aaagccacat |
| 4141 | tggagctccc ttctacccac taaaaaataa ccaattttta cattttttga gggggagtga |
| 4201 | gttttaggaa aggggaatta agattccagg gagagctctg gggatagaac agggcgcaga |
| 4261 | ttccatctct ccccaagccc ctttttagtg actaagtcaa ggccccaact cccctccccc |
| 4321 | accctacgct gagcttattc gagttcattc gtactaataa tccctcctgc ggcttcctca |
| 4381 | ttgttgctgt tttaggccac cccagctcag ccaatgattc ctttccctct gaatgtcagt |
| 4441 | tttgttttta aaagtcactt gcttagttga tgtcagcgta tgtgtatttg gtggggaaaa |
| 4501 | cctaatttcg gggatttctg tggtaggtaa taggagaaga aagggcactg ggggctgttc |
| 4561 | tccttccttc cctgggctgt atccatggac tcctggaagg cacagagaag ggagctataa |
| 4621 | gaggatgtga agttttaaaa cctgaaattg ttttttaaag cacttaagca cctccatatt |
| 4681 | atgacttggt gggtcacccc ttagcttcct ccctctccca ccaagactat gagaacttca |
| 4741 | gctgatagct gggggctccc cagatgagga tgcagggatt tgggagcagt ggaagagggt |
| 4801 | gcccaacctt gggttggacc aacccttggc tcgcagctca actctgcttc ccgcattcct |
| 4861 | gctccacgtg tcccagcttc tcccctgtga cgggaaggca ggtgtgactc caggctctgc |
| 4921 | actggttctt cttggttcct cccaccaggc cctttgttcc tcatgtcccc atgtttctct |
| 4981 | ccctctgcgt cttagcacct ttcttctgtt caaagttttc tgtaaatttt ctcttttttt |
| 5041 | ctttctttct tttttttttt tttataaatt aatttgcttt cagttccaaa aaaaaaaaaa |
| 5101 | aaaaaa |

SEQ ID NO: 65 Human SMARCC2 Amino Acid Sequence Isoform A
(NP_003066.2)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdii krhqgtvted knnashvvyp |
| 181 | vpgnleeeew vrpvmkrdkq vllhwgyypd sydtwipase ieasvedapt pekprkvhak |
| 241 | wildtdtfne wmneedyevn ddknpvsrrk kisaktltde vnspdsdrrd kkggnykkrk |
| 301 | rspspsptpe akkknakkgp stpytkskrg hreeeqedlt kdmdepspvp nveevtlpkt |
| 361 | vntkkdsesa pvkggtmtdl deqedesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpktpq qtsasqqmln fpdkgkekpt dmqnfglrtd mytkknvpsk skaaasatre |
| 601 | wteqetllll ealemykddw nkvsehvgsr tqdecilhfl rlpiedpyle dseaslgpla |
| 661 | yqpipfsqsg npvmstvafl asvvdprvas aaaksaleef skmkeevpta lveahvrkve |
| 721 | eaakvtgkad pafglessgi agttsdeper ieesgndear vegqatdekk epkepreggg |
| 781 | aieeeakekt seapkkdeek gkegdsekes eksdgdpivd pekekepkeg qeevlkevve |
| 841 | segerktkve rdigegnlst aaaaalaaaa vkakhlaave erkikslval lvetqmkkle |
| 901 | iklrhfeele timdrereal eyqrqqllad rqafhmeqlk yaemrarqqh fqqmhqqqqq |
| 961 | pppalppgsq pipptgaagp pavhglavap asvvpapags gappgslgps eqigqagsta |
| 1021 | gpqqqqpaga pqpgavppgv pppgphgpsp fpnqqtppsm mpgavpgsgh pgvagnaplg |
| 1081 | lpfgmppppp ppapsiipfg sladsisinl pappnlhghh hhlpfapgtl pppnlpvsma |
| 1141 | nplhpnlpat ttmpsslplg pglgsaaaqs paivaavqgn llpsasplpd pgtplppdpt |
| 1201 | apspgtvtpv pppq |

SEQ ID NO: 66 Human SMARCC2 cDNA Sequence Variant 2 (NM_139067.3,
CDS: 114-3506)

| | |
|---|---|
| 1 | ggaggcggcg gccgcggcgg cgggaggcgg cgggaggcgg gcggaggagg aggcggagga |
| 61 | ggcgggagct gagctgagtg gggcgggcgg cggcggggcc cgagccggag aagatggcgg |
| 121 | tgcggaagaa ggacggcggc cccaacgtga agtactacga ggccgcggac accgtgaccc |
| 181 | agttcgacaa cgtgcggctg tggctcggca agaactacaa gaagtatata caagctgaac |
| 241 | cacccaccaa caagtccctg tctagcctgg ttgtacagtt gctacaattt caggaagaag |
| 301 | tttttggcaa acatgtcagc aatgcaccgc tcactaaact gccgatcaaa tgtttcctag |
| 361 | atttcaaagc gggaggctcc ttgtgccaca ttcttgcagc tgcctacaaa ttcaagagtg |
| 421 | accagggatg gcggcgttac gatttccaga atccatcacg catggaccgc aatgtggaaa |
| 481 | tgtttatgac cattgagaag tccttggtgc agaataattg cctgtctcga cctaacattt |
| 541 | ttctgtgccc agaaattgag cccaaactac tagggaaatt aaaggacatt atcaagagac |
| 601 | accagggaac agtcactgag gataagaaca atgcctccca tgttgtgtat cctgtcccgg |
| 661 | ggaatctaga agaagaggaa tgggtacgac cagtcatgaa gagggataag caggttcttc |
| 721 | tgcactgggg ctactatcct gacagttacg acacgtggat cccagcgagt gaaattgagg |
| 781 | catctgtgga agatgctcca actcctgaga aacctaggaa ggttcatgca aagtggatcc |
| 841 | tggacaccga caccttcaat gaatggatga atgaggaaga ctatgaagta aatgatgaca |
| 901 | aaaaccctgt ctcccgccga aagaagattt cagccaagac actgacagat gaggtgaaca |
| 961 | gcccagattc agatcgacgg gacaagaagg ggggaaacta taagaagagg aagcgctccc |
| 1021 | cctctccttc accaacccca gaagcaaaga agaaaaatgc taagaaaggt ccctcaacac |
| 1081 | cttacactaa gtcaaagcgt ggccacagag aagaggagca agaagacctg acaaaggaca |
| 1141 | tggacgagcc ctcaccagtc cccaatgtag aagaggtgac acttcccaaa acagtcaaca |
| 1201 | caaagaaaga ctcagagtcg gccccagtca aaggcggcac catgaccgac ctggatgaac |
| 1261 | aggaagatga aagcatggag acgacgggca aggatgagga tgagaacagt acggggaaca |
| 1321 | agggagagca gaccaagaat ccagacctgc atgaggacaa tgtgactgaa cagacccacc |
| 1381 | acatcatcat tcccagctac gctgcctggt ttgactacaa tagtgttcat gccattgagc |
| 1441 | ggagggctct ccccgagttc ttcaacggca agaacaagtc caagactcca gagatctacc |
| 1501 | tggcctatcg aaactttatg attgacactt accgactgaa cccccaagag tatcttacct |
| 1561 | ctaccgcctg ccgccgaaac ctagcgggtg atgtctgtgc catcatgagg gtccatgcct |
| 1621 | tcctagaaca gtggggtctt attaactacc aggtggatgc tgagagtcga ccaaccccaa |
| 1681 | tggggcctcc gcctacctct cacttccatg tcttggctga cacaccatca gggctggtgc |
| 1741 | ctctgcagcc caagacacct cagggccgcc aggttgatgc tgataccaag gctgggcgaa |
| 1801 | agggcaaaga gctggatgac ctggtgccag agacggctaa gggcaagcca gagctgcaga |
| 1861 | cctctgcttc ccaacaaatg ctcaactttc ctgacaaagg caaagagaaa ccaacagaca |
| 1921 | tgcaaaactt tgggctgcgc acagacatgt acacaaaaaa gaatgttccc tccaagagca |
| 1981 | aggctgcagc cagtgccact cgtgagtgga cagaacagga aaccctgctt ctcctggagg |
| 2041 | cactggaaat gtacaaagat gactggaaca aagtgtccga gcatgtggga agccgcacac |
| 2101 | aggacgagtg catcttgcat tttcttcgtc ttcccattga agacccatac ctggaggact |
| 2161 | cagaggcctc cctaggcccc ctggcctacc aacccatccc cttcagtcag tcgggcaacc |
| 2221 | ctgttatgag cactgttgcc ttcctggcct ctgtcgtcga tccccgagtc gcctctgctg |
| 2281 | ctgcaaagtc agccctagag gagttctcca aaatgaagga agaggtaccc acggccttgg |
| 2341 | tggaggccca tgttcgaaaa gtggaagaag cagccaaagt aacaggcaag gcggaccctg |
| 2401 | ccttcggtct ggaaagcagt ggcattgcag gaaccacctc tgatgagcct gagcggattg |
| 2461 | aggagagcgg gaatgacgag gctcgggtgg aaggccaggc cacagatgag aagaaggagc |
| 2521 | ccaaggaacc ccgagaagga gggggtgcta tagaggagga agcaaaagag aaaaccagcg |
| 2581 | aggctcccaa gaaggatgag gagaaaggga aagaaggcga cagtgagaag gagtccgaga |
| 2641 | agagtgatgg agacccaata gtcgatcctg agaaggagaa ggagccaaag gaagggcagg |
| 2701 | aggaagtgct gaaggaagtg gtggagtctg agggggaaag gaagacaaag gtggagcggg |
| 2761 | acattggcga gggcaacctc tccaccgctg ctgccgccgc cctggccgcc gccgcagtga |
| 2821 | aagctaagca cttggctgct gttgaggaaa ggaagatcaa atctttggtg gccctgctgg |
| 2881 | tggagaccca gatgaaaaag ttggagatca aacttcggca ctttgaggag ctggagacta |
| 2941 | tcatggaccg ggagcgagaa gcactggagt atcagaggca gcagctcctg gccgacagac |
| 3001 | aagccttcca catggagcag ctgaagtatg cggagatgag ggctcggcag cagcacttcc |
| 3061 | aacagatgca ccaacagcag cagcagccac caccagccct gcccccaggc tcccagccta |
| 3121 | tccccccaac aggggctgct gggccacccg cagtccatgg cttggctgtg gctccagcct |
| 3181 | ctgtagtccc tgctcctgct ggcagtgggg cccctccagg aagtttgggc ccttctgaac |
| 3241 | agattgggca ggcagggtca actgcagggc cacagcagca gcaaccagct ggagcccccc |
| 3301 | agcctggggc agtcccacca ggggttcccc cccctggacc ccatggcccc tcaccgttcc |
| 3361 | ccaaccaaca aactcctccc tcaatgatgc caggggcagt gccaggcagc gggcacccag |
| 3421 | gcgtggcgga cccaggcacc cccctgcctc cagaccccac agccccgagc ccaggcacgg |
| 3481 | tcacccctgt gccacctcca cagtgaggag ccagccagac atctctcccc ctcaccccct |
| 3541 | gtggacatca cggttccagg aacagccctt cccccaccac tgggaccctc cccagcctgg |
| 3601 | agagttcatc actacgtaag gaaagctcct tccgcccctc caaagccctc accatgccta |
| 3661 | acagaggcat gcatttttat atcagattat tcaaggactt ctgtttaaaa gatgtttata |
| 3721 | atgtctggga gagaggatag gatgggaatg ctgccctaaa ggaagggctg gtgaaaggtg |
| 3781 | tttatacaag gttctattaa ccacttctaa gggtacacct ccctccaaac tactgcattt |
| 3841 | tctatggatt aaaaaaaaaa aaaaaaagta gattttaaaa agccacattg gagctccctt |
| 3901 | ctacccacta aaaaataacc aatttttaca ttttttgagg gggagtgagt tttaggaaag |
| 3961 | gggaattaag attccaggga gagctctggg gatagaacag ggcgcagatt ccatctctcc |
| 4021 | ccaagcccct ttttagtgac taagtcaagg ccccaactcc cctcccccac cctacgctga |
| 4081 | gcttattcga gttcattcgt actaataatc cctcctgcgg cttcctcatt gttgctgttt |
| 4141 | taggccaccc cagctcagcc aatgattcct ttccctctga atgtcagttt tgtttttaaa |
| 4201 | agtcacttgc ttagttgatg tcagcgtatg tgtatttggt ggggaaaacc taatttcggg |
| 4261 | gatttctgtg gtaggtaata ggagaagaaa gggcactggg ggctgttctc cttccttccc |
| 4321 | tgggctgtat ccatggactc ctggaaggca cagagaaggg agctataaga ggatgtgaag |
| 4381 | ttttaaaacc tgaaattgtt ttttaaagca cttaagcacc tccatattat gacttggtgg |
| 4441 | gtcacccctt agcttcctcc ctctcccacc aagactatga gaacttcagc tgatagctgg |
| 4501 | gggctcccca gatgaggatg cagggatttg ggagcagtgg aagagggtgc ccaaccttgg |
| 4561 | gttggaccaa cccttggctc gcagctcaac tctgcttccc gcattcctgc tccacgtgtc |
| 4621 | ccagcttctc ccctgtgacg ggaaggcagg tgtgactcca ggctctgcac tggttcttct |
| 4681 | tggttcctcc caccaggccc tttgttcctc atgtccccat gtttctctcc ctctgcgtct |
| 4741 | tagcaccttt cttctgttca aagttttctg taaattttct ctttttttct ttctttcttt |
| 4801 | tttttttttt tataaattaa tttgctttca gttccaaaaa aaaaaaaaaa aaaa |

SEQ ID NO: 67 Human SMARCC2 Amino Acid Sequence Isoform B
(NP_620706.1)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdii krhqgtvted knnashvvyp |
| 181 | vpgnleeeew vrpvmkrdkq vllhwgyypd sydtwipase ieasvedapt pekprkvhak |
| 241 | wildtdtfne wmneedyevn ddknpvsrrk kisaktltde vnspdsdrrd kkggnykkrk |
| 301 | rspspsptpe akkknakkgp stpytkskrg hreeeqedlt kdmdepspvp nveevtlpkt |
| 361 | vntkkdsesa pvkggtmtdl deqedesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpktpq grqvdadtka grkgkelddl vpetakgkpe lqtsasqqml nfpdkgkekp |
| 601 | tdmqnfglrt dmytkknvps kskaaasatr ewteqetlll lealemykdd wnkvsehvgs |
| 661 | rtqdecilhf lrlpiedpyl edseaslgpl ayqpipfsqs gnpvmstvaf lasvvdprva |
| 721 | saaaksalee fskmkeevpt alveahvrkv eeaakvtgka dpafglessg iagttsdepe |
| 781 | rieesgndea rvegqatdek kepkepregg gaieeeakek tseapkkdee kgkegdseke |
| 841 | seksdgdpiv dpekekepke gqeevlkevv esegerktkv erdigegnls taaaaalaaa |
| 901 | avkakhlaav eerkikslva llvetqmkkl eiklrhfeel etimdrerea leyqrqqlla |
| 961 | drqafhmeql kyaemrarqq hfqqmhqqqq qpppalppgs qpipptgaag ppavhglava |
| 1021 | pasvvpapag sgappgslgp seqigqagst agpqqqqpag apqpgavppg vpppgphgps |
| 1081 | pfpnqqtpps mmpgavpgsg hpgvadpgtp lppdptapsp gtvtpvpppq |

SEQ ID NO: 68 Human SMARCC2 cDNA Sequence Variant 3 (NM_00113020.2,
CDS: 114-3572)

| | |
|---|---|
| 1 | ggaggcggcg gccgcggcgg cgggaggcgg cgggaggcgg gcggaggagg aggcggagga |
| 61 | ggcgggagct gagctgagtg gggcgggcgg cggcggggcc cgagccggag aagatggcgg |
| 121 | tgcggaagaa ggacggcggc cccaacgtga agtactacga ggccgcggac accgtgaccc |
| 181 | agttcgacaa cgtgcggctg tggctcggca agaactacaa gaagtatata caagctgaac |
| 241 | cacccaccaa caagtccctg tctagcctgg ttgtacagtt gctacaattt caggaagaag |
| 301 | tttttggcaa acatgtcagc aatgcaccgc tcactaaact gccgatcaaa tgtttcctag |
| 361 | atttcaaagc gggaggctcc ttgtgccaca ttcttgcagc tgcctacaaa ttcaagagtg |
| 421 | accagggatg gcggcgttac gatttccaga atccatcacg catggaccgc aatgtggaaa |
| 481 | tgtttatgac cattgagaag tccttggtgc agaataattg cctgtctcga cctaacattt |
| 541 | ttctgtgccc agaaattgag cccaaactac tagggaaatt aaaggacatt atcaagagac |
| 601 | accagggaac agtcactgag gataagaaca atgcctccca tgttgtgtat cctgtcccgg |
| 661 | ggaatctaga agaagaggaa tgggtacgac cagtcatgaa gagggataag caggttcttc |
| 721 | tgcactgggg ctactatcct gacagttacg acacgtggat cccagcgagt gaaattgagg |
| 781 | catctgtgga agatgctcca actcctgaga aacctaggaa ggttcatgca aagtggatcc |
| 841 | tggacaccga caccttcaat gaatggatga atgaggaaga ctatgaagta aatgatgaca |
| 901 | aaaaccctgt ctcccgccga aagaagattt cagccaagac actgacagat gaggtgaaca |
| 961 | gcccagattc agatcgacgg gacaagaagg ggggaaacta taagaagagg aagcgctccc |
| 1021 | cctctccttc accaacccca gaagcaaaga agaaaaatgc taagaaaggt ccctcaacac |
| 1081 | cttacactaa gtcaaagcgt ggccacagag aagaggagca agaagacctg acaaaggaca |
| 1141 | tggacgagcc ctcaccagtc cccaatgtag aagaggtgac acttcccaaa acagtcaaca |
| 1201 | caaagaaaga ctcagagtcg gccccagtca aaggcggcac catgaccgac ctggatgaac |
| 1261 | aggaagatga aagcatggag acgacgggca aggatgagga tgagaacagt acggggaaca |
| 1321 | agggagagca gaccaagaat ccagacctgc atgaggacaa tgtgactgaa cagacccacc |
| 1381 | acatcatcat tcccagctac gctgcctggt ttgactacaa tagtgttcat gccattgagc |
| 1441 | ggagggctct ccccgagttc ttcaacggca agaacaagtc caagactcca gagatctacc |
| 1501 | tggcctatcg aaactttatg attgacactt accgactgaa cccccaagag tatcttacct |
| 1561 | ctaccgcctg ccgccgaaac ctagcgggtg atgtctgtgc catcatgagg gtccatgcct |
| 1621 | tcctagaaca gtggggtctt attaactacc aggtggatgc tgagagtcga ccaaccccaa |
| 1681 | tggggcctcc gcctacctct cacttccatg tcttggctga cacaccatca gggctggtgc |
| 1741 | ctctgcagcc caagacacct cagggccgcc aggttgatgc tgataccaag gctgggcgaa |
| 1801 | agggcaaaga gctggatgac ctggtgccag agacggctaa gggcaagcca gagctgcaga |
| 1861 | cctctgcttc ccaacaaatg ctcaactttc ctgacaaagg caaagagaaa ccaacagaca |
| 1921 | tgcaaaactt tgggctgcgc acagacatgt acacaaaaaa gaatgttccc tccaagagca |
| 1981 | aggctgcagc cagtgccact cgtgagtgga cagaacagga aaccctgctt ctcctggagg |
| 2041 | cactggaaat gtacaaagat gactggaaca aagtgtccga gcatgtggga agccgcacac |
| 2101 | aggacgagtg catcttgcat tttcttcgtc ttcccattga agacccatac ctggaggact |
| 2161 | cagaggcctc cctaggcccc ctggcctacc aacccatccc cttcagtcag tcgggcaacc |
| 2221 | ctgttatgag cactgttgcc ttcctggcct ctgtcgtcga tccccgagtc gcctctgctg |
| 2281 | ctgcaaagtc agccctagag gagttctcca aaatgaagga agaggtaccc acggccttgg |
| 2341 | tggaggccca tgttcgaaaa gtggaagaag cagccaaagt aacaggcaag gcggaccctg |
| 2401 | ccttcggtct ggaaagcagt ggcattgcag gaaccacctc tgatgagcct gagcggattg |
| 2461 | aggagagcgg gaatgacgag gctcgggtgg aaggccaggc cacagatgag aagaaggagc |
| 2521 | ccaaggaacc ccgagaagga gggggtgcta tagaggagga agcaaaagag aaaaccagcg |
| 2581 | aggctcccaa gaaggatgag gagaaaggga aagaaggcga cagtgagaag gagtccgaga |
| 2641 | agagtgatgg agacccaata gtcgatcctg agaaggagaa ggagccaaag gaagggcagg |
| 2701 | aggaagtgct gaaggaagtg gtggagtctg agggggaaag gaagacaaag gtggagcggg |
| 2761 | acattggcga gggcaacctc tccaccgctg ctgccgccgc cctggccgcc gccgcagtga |
| 2821 | aagctaagca cttggctgct gttgaggaaa ggaagatcaa atctttggtg gccctgctgg |
| 2881 | tggagaccca gatgaaaaag ttggagatca aacttcggca ctttgaggag ctggagacta |
| 2941 | tcatggaccg ggagcgagaa gcactggagt atcagaggca gcagctcctg gccgacagac |
| 3001 | aagccttcca catggagcag ctgaagtatg cggagatgag ggctcggcag cagcacttcc |
| 3061 | aacagatgca ccaacagcag cagcagccac caccagccct gcccccaggc tcccagccta |
| 3121 | tccccccaac aggggctgct gggccacccg cagtccatgg cttggctgtg gctccagcct |
| 3181 | ctgtagtccc tgctcctgct ggcagtgggg cccctccagg aagtttgggc ccttctgaac |
| 3241 | agattgggca ggcagggtca actgcagggc cacagcagca gcaaccagct ggagcccccc |
| 3301 | agcctggggc agtcccacca ggggttcccc cccctggacc ccatggcccc tcaccgttcc |
| 3361 | ccaaccaaca aactcctccc tcaatgatgc caggggcagt gccaggcagc gggcacccag |
| 3421 | gcgtggcggc ccaaagccct gccattgtgg cagctgttca gggcaacctc ctgcccagtg |
| 3481 | ccagcccact gccagaccca ggcacccccc tgcctccaga ccccacagcc ccgagcccag |
| 3541 | gcacggtcac ccctgtgcca cctccacagt gaggagccag ccagacatct ctccccctca |
| 3601 | ccccctgtgg acatcacggt tccaggaaca gcccttcccc caccactggg accctcccca |
| 3661 | gcctggagag ttcatcacta cgtaaggaaa gctccttccg cccctccaaa gccctcacca |
| 3721 | tgcctaacag aggcatgcat ttttatatca gattattcaa ggacttctgt ttaaaagatg |
| 3781 | tttataatgt ctgggagaga ggataggatg ggaatgctgc cctaaaggaa gggctggtga |
| 3841 | aaggtgttta tacaaggttc tattaaccac ttctaagggt acacctccct ccaaactact |
| 3901 | gcattttcta tggattaaaa aaaaaaaaaa aaagtagatt ttaaaaagcc acattggagc |
| 3961 | tcccttctac ccactaaaaa ataaccaatt tttacatttt ttgaggggga gtgagtttta |
| 4021 | ggaaagggga attaagattc cagggagagc tctggggata gaacagggcg cagattccat |
| 4081 | ctctccccaa gccccttttt agtgactaag tcaaggcccc aactcccctc ccccacccta |
| 4141 | cgctgagctt attcgagttc attcgtacta ataatccctc ctgcggcttc ctcattgttg |
| 4201 | ctgttttagg ccaccccagc tcagccaatg attcctttcc ctctgaatgt cagttttgtt |
| 4261 | tttaaaagtc acttgcttag ttgatgtcag cgtatgtgta tttggtgggg aaaacctaat |
| 4321 | ttcggggatt tctgtggtag gtaataggag aagaaagggc actgggggct gttctccttc |
| 4381 | cttccctggg ctgtatccat ggactcctgg aaggcacaga gaagggagct ataagaggat |
| 4441 | gtgaagtttt aaaacctgaa attgtttttt aaagcactta agcacctcca tattatgact |
| 4501 | tggtgggtca ccccttagct tcctccctct cccaccaaga ctatgagaac ttcagctgat |
| 4561 | agctgggggc tccccagatg aggatgcagg gatttgggag cagtggaaga gggtgcccaa |
| 4621 | ccttgggttg gaccaaccct tggctcgcag ctcaactctg cttcccgcat tcctgctcca |
| 4681 | cgtgtcccag cttctcccct gtgacgggaa ggcaggtgtg actccaggct ctgcactggt |
| 4741 | tcttcttggt tcctcccacc aggccctttg ttcctcatgt ccccatgttt ctctccctct |
| 4801 | gcgtcttagc acctttcttc tgttcaaagt tttctgtaaa ttttctcttt ttttctttct |
| 4861 | ttcttttttt tttttttata aattaatttg ctttcagttc caaaaaaaaa aaaaaaaaaa |

SEQ ID NO: 69 Human SMARCC2 Amino Acid Sequence Isoform C
(NP_001123892.1)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdii krhqgtvted knnashvvyp |
| 181 | vpgnleeeew vrpvmkrdkq vllhwgyypd sydtwipase ieasvedapt pekprkvhak |
| 241 | wildtdtfne wmneedyevn ddknpvsrrk kisaktltde vnspdsdrrd kkggnykkrk |
| 301 | rspspsptpe akkknakkgp stpytkskrg hreeegedlt kdmdepspvp nveevtlpkt |
| 361 | vntkkdsesa pvkggtmtdl deqedesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpktpq grqvdadtka grkgkelddl vpetakgkpe lqtsasqqml nfpdkgkekp |
| 601 | tdmqnfglrt dmytkknvps kskaaasatr ewteqetlll lealemykdd wnkvsehvgs |
| 661 | rtqdecilhf lrlpiedpyl edseaslgpl ayqpipfsqs gnpvmstvaf lasvvdprva |
| 721 | saaaksalee fskmkeevpt alveahvrkv eeaakvtgka dpafglessg iagttsdepe |
| 781 | rieesgndea rvegqatdek kepkepregg gaieeeakek tseapkkdee kgkegdseke |
| 841 | seksdgdpiv dpekekepke gqeevlkevv esegerktkv erdigegnls taaaaalaaa |
| 901 | avkakhlaav eerkikslva llvetqmkkl eiklrhfeel etimdrerea leyqrqqlla |
| 961 | drqafhmeql kyaemrarqq hfqqmhqqqq qpppalppgs qpipptgaag ppavhglava |
| 1021 | pasvvpapag sgappgslgp seqigqagst agpqqqqpag apqpgavppg vpppgphgps |
| 1081 | pfpnqqtpps mmpgavpgsg hpgvaaqspa ivaavqgnll psasplpdpg tplppdptap |
| 1141 | spgtvtpvpp pq |

SEQ ID NO: 70 Human SMARCC2 cDNA Sequence Variant 4 (NM_001330288.1,
CDS: 114-3851)

| | |
|---|---|
| 1 | ggaggcggcg gccgcggcgg cgggaggcgg cgggaggcgg gcggaggagg aggcggagga |
| 61 | ggcgggagct gagctgagtg gggcgggcgg cggcggggcc cgagccggag aagatggcgg |
| 121 | tgcggaagaa ggacggcggc cccaacgtga agtactacga ggccgcggac accgtgaccc |
| 181 | agttcgacaa cgtgcggctg tggctcggca agaactacaa gaagtatata caagctgaac |
| 241 | cacccaccaa caagtccctg tctagcctgg ttgtacagtt gctacaattt caggaagaag |
| 301 | tttttggcaa acatgtcagc aatgcaccgc tcactaaact gccgatcaaa tgtttcctag |
| 361 | atttcaaagc gggaggctcc ttgtgccaca ttcttgcagc tgcctacaaa ttcaagagtg |
| 421 | accagggatg gcggcgttac gatttccaga atccatcacg catggaccgc aatgtggaaa |
| 481 | tgtttatgac cattgagaag tccttggtgc agaataattg cctgtctcga cctaacattt |
| 541 | ttctgtgccc agaaattgag cccaaactac tagggaaatt aaaggacatt atcaagagac |
| 601 | accagggaac agtcactgag gataagaaca atgcctccca tgttgtgtat cctgtcccgg |
| 661 | ggaatctaga agaagaggaa tgggtacgac cagtcatgaa gagggataag caggttcttc |
| 721 | tgcactgggg ctactatcct gacagttacg acacgtggat cccagcgagt gaaattgagg |
| 781 | catctgtgga agatgctcca actcctgaga aacctaggaa ggttcatgca aagtggatcc |
| 841 | tggacaccga caccttcaat gaatggatga atgaggaaga ctatgaagta aatgatgaca |
| 901 | aaaaccctgt ctcccgccga aagaagattt cagccaagac actgacagat gaggtgaaca |
| 961 | gcccagattc agatcgacgg gacaagaagg ggggaaacta taagaagagg aagcgctccc |
| 1021 | cctctccttc accaacccca gaagcaaaga agaaaaatgc taagaaaggt ccctcaacac |
| 1081 | cttacactaa gtcaaagcgt ggccacagag aagaggagca agaagacctg acaaaggaca |
| 1141 | tggacgagcc ctcaccagtc cccaatgtag aagaggtgac acttcccaaa acagtcaaca |
| 1201 | caaagaaaga ctcagagtcg gccccagtca aaggcggcac catgaccgac ctggatgaac |
| 1261 | aggaagatga aagcatggag acgacgggca aggatgagga tgagaacagt acggggaaca |
| 1321 | agggagagca gaccaagaat ccagacctgc atgaggacaa tgtgactgaa cagacccacc |
| 1381 | acatcatcat tcccagctac gctgcctggt ttgactacaa tagtgttcat gccattgagc |
| 1441 | ggagggctct ccccgagttc ttcaacggca agaacaagtc caagactcca gagatctacc |
| 1501 | tggcctatcg aaactttatg attgacactt accgactgaa cccccaagag tatcttacct |
| 1561 | ctaccgcctg ccgccgaaac ctagcgggtg atgtctgtgc catcatgagg gtccatgcct |
| 1621 | tcctagaaca gtggggtctt attaactacc aggtggatgc tgagagtcga ccaaccccaa |
| 1681 | tggggcctcc gcctacctct cacttccatg tcttggctga cacaccatca gggctggtgc |
| 1741 | ctctgcagcc caagacacct cagggccgcc aggttgatgc tgataccaag gctgggcgaa |
| 1801 | agggcaaaga gctggatgac ctggtgccag agacggctaa gggcaagcca gagctgcaga |
| 1861 | cctctgcttc ccaacaaatg ctcaactttc ctgacaaagg caaagagaaa ccaacagaca |
| 1921 | tgcaaaactt tgggctgcgc acagacatgt acacaaaaaa gaatgttccc tccaagagca |
| 1981 | aggctgcagc cagtgccact cgtgagtgga cagaacagga aaccctgctt ctcctggagg |
| 2041 | cactggaaat gtacaaagat gactggaaca aagtgtccga gcatgtggga agccgcacac |
| 2101 | aggacgagtg catcttgcat tttcttcgtc ttcccattga agacccatac ctggaggact |
| 2161 | cagaggcctc cctaggcccc ctggcctacc aacccatccc cttcagtcag tcgggcaacc |
| 2221 | ctgttatgag cactgttgcc ttcctggcct ctgtcgtcga tccccgagtc gcctctgctg |
| 2281 | ctgcaaagtc agccctagag gagttctcca aaatgaagga agaggtaccc acggccttgg |
| 2341 | tggaggccca tgttcgaaaa gtggaagaag cagccaaagt aacaggcaag gcggaccctg |
| 2401 | ccttcggtct ggaaagcagt ggcattgcag gaaccacctc tgatgagcct gagcggattg |
| 2461 | aggagagcgg gaatgacgag gctcgggtgg aaggccaggc cacagatgag aagaaggagc |
| 2521 | ccaaggaacc ccgagaagga gggggtgcta tagaggagga agcaaaagag aaaaccagcg |
| 2581 | aggctcccaa gaaggatgag gagaaaggga aagaaggcga cagtgagaag gagtccgaga |
| 2641 | agagtgatgg agacccaata gtcgatcctg agaaggagaa ggagccaaag gaagggcagg |
| 2701 | aggaagtgct gaaggaagtg gtggagtctg agggggaaag gaagacaaag gtggagcggg |
| 2761 | acattggcga gggcaacctc tccaccgctg ctgccgccgc cctggccgcc gccgcagtga |
| 2821 | aagctaagca cttggctgct gttgaggaaa ggaagatcaa atctttggtg gccctgctgg |
| 2881 | tggagaccca gatgaaaaag ttggagatca aacttcggca ctttgaggag ctggagacta |
| 2941 | tcatggaccg ggagcgagaa gcactggagt atcagaggca gcagctcctg gccgacagac |
| 3001 | aagccttcca catggagcag ctgaagtatg cggagatgag ggctcggcag cagcacttcc |
| 3061 | aacagatgca ccaacagcag cagcagccac caccagccct gcccccaggc tcccagccta |
| 3121 | tccccccaac aggggctgct gggccacccg cagtccatgg cttggctgtg gctccagcct |
| 3181 | ctgtagtccc tgctcctgct ggcagtgggg cccctccagg aagtttgggc ccttctgaac |
| 3241 | agattgggca ggcagggtca actgcagggc cacagcagca gcaaccagct ggagcccccc |
| 3301 | agcctggggc agtcccacca ggggttcccc cccctggacc ccatggcccc tcaccgttcc |
| 3361 | ccaaccaaca aactcctccc tcaatgatgc caggggcagt gccaggcagc gggcacccag |
| 3421 | gcgtggcggg taatgctcct ttgggtttgc cttttggcat gccgcctcct cctcctcctc |
| 3481 | ctgctccatc catcatccca tttggtagtc tagctgactc catcagtatt aacctccccg |
| 3541 | ctcctcctaa cctgcatggg catcaccacc atctcccgtt cgccccgggc actctccccc |
| 3601 | cacctaacct gcctgtgtcc atggcgaacc ctctacatcc taacctgccg gcgaccacca |
| 3661 | ccatgccatc ttccttgcct ctcgggccgg ggctcggatc cgccgcagcc caaagccctg |
| 3721 | ccattgtggc agctgttcag ggcaacctcc tgcccagtgc cagcccactg ccagacccag |
| 3781 | gcacccccct gcctccagac cccacagccc cgagcccagg cacggtcacc cctgtgccac |
| 3841 | ctccacagtg aggagccagc cagacatctc tccccctcac cccctgtgga catcacggtt |
| 3901 | ccaggaacag cccttccccc accactggga ccctccccag cctggagagt tcatcactac |
| 3961 | gtaaggaaag ctccttccgc ccctccaaag ccctcaccat gcctaacaga ggcatgcatt |
| 4021 | tttatatcag attattcaag gacttctgtt taaaagatgt ttataatgtc tgggagagag |
| 4081 | gataggatgg gaatgctgcc ctaaaggaag ggctggtgaa aggtgtttat acaaggttct |
| 4141 | attaaccact tctaagggta cacctccctc caaactactg cattttctat ggattaaaaa |
| 4201 | aaaaaaaaaa aagtagattt taaaaagcca cattggagct cccttctacc cactaaaaaa |
| 4261 | taaccaattt ttacattttt tgagggggag tgagttttag gaaaggggaa ttaagattcc |
| 4321 | agggagagct ctggggatag aacagggcgc agattccatc tctccccaag ccccttttta |
| 4381 | gtgactaagt caaggcccca actcccctcc cccaccctac gctgagctta ttcgagttca |
| 4441 | ttcgtactaa taatccctcc tgcggcttcc tcattgttgc tgttttaggc caccccagct |
| 4501 | cagccaatga ttcctttccc tctgaatgtc agttttgttt ttaaaagtca cttgcttagt |
| 4561 | tgatgtcagc gtatgtgtat ttggtgggga aaacctaatt tcggggattt ctgtggtagg |
| 4621 | taataggaga agaaagggca ctgggggctg ttctccttcc ttccctgggc tgtatccatg |
| 4681 | gactcctgga aggcacagag aagggagcta taagaggatg tgaagtttta aaacctgaaa |
| 4741 | ttgtttttta aagcacttaa gcacctccat attatgactt ggtgggtcac cccttagctt |
| 4801 | cctccctctc ccaccaagac tatgagaact tcagctgata gctgggggct ccccagatga |
| 4861 | ggatgcaggg atttgggagc agtggaagag ggtgcccaac cttgggttgg accaaccctt |
| 4921 | ggctcgcagc tcaactctgc ttcccgcatt cctgctccac gtgtcccagc ttctcccctg |
| 4981 | tgacgggaag gcaggtgtga ctccaggctc tgcactggtt cttcttggtt cctcccacca |
| 5041 | ggccctttgt tcctcatgtc cccatgtttc tctccctctg cgtcttagca cctttcttct |
| 5101 | gttcaaagtt ttctgtaaat tttctctttt tttctttctt tctttttttt ttttttataa |
| 5161 | attaatttgc tttcagttcc aaaaaaaaaa aaaaaaaaa |

SEQ ID NO: 71 Human SMARCC2 Amino Acid Sequence Isoform D
(NP_001317217.1)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdii krhqgtvted knnashvvyp |
| 181 | vpgnleeeew vrpvmkrdkq vllhwgyypd sydtwipase ieasvedapt pekprkvhak |
| 241 | wildtdtfne wmneedyevn ddknpvsrrk kisaktltde vnspdsdrrd kkggnykkrk |
| 301 | rspspsptpe akkknakkgp stpytkskrg hreeeqedlt kdmdepspvp nveevtlpkt |
| 361 | vntkkdsesa pvkggtmtdl deqedesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpktpq grqvdadtka grkgkelddl vpetakgkpe lqtsasqqml nfpdkgkekp |
| 601 | tdmqnfglrt dmytkknvps kskaaasatr ewteqetlll lealemykdd wnkvsehvgs |
| 661 | rtqdecilhf lrlpiedpyl edseaslgpl ayqpipfsqs gnpvmstvaf lasvvdprva |
| 721 | saaaksalee fskmkeevpt alveahvrkv eeaakvtgka dpafglessg iagttsdepe |
| 781 | rieesgndea rvegqatdek kepkepregg gaieeeakek tseapkkdee kgkegdseke |
| 841 | seksdgdpiv dpekekepke gqeevlkevv esegerktkv erdigegnls taaaaalaaa |
| 901 | avkakhlaav eerkikslva llvetqmkkl eiklrhfeel etimdrerea leyqrqqlla |
| 961 | drqafhmeql kyaemrarqq hfqqmhqqqq qpppalppgs qpipptgaag ppavhglava |
| 1021 | pasvvpapag sgappgslgp seqigqagst agpqqqqpag apqpgavppg vpppgphgps |
| 1081 | pfpnqqtpps mmpgavpgsg hpgvagnapl glpfgmpppp pppapsiipf gsladsisin |
| 1141 | lpappnlhgh hhhlpfapgt lpppnlpvsm anplhpnlpa tttmpsslpl gpglgsaaaq |
| 1201 | spaivaavqg nllpsasplp dpgtplppdp tapspgtvtp vpppq |

SEQ ID NO: 72 Mouse SMARCC2 cDNA Sequence Variant 1 (NM_001114097.1,
CDS: 92-3733)

| | |
|---|---|
| 1 | gtggcggcgg gaggcggcgg gaggcgggcg gaggaggagg cgggagctga gctgagcggg |
| 61 | gcgggcggcg gcggggcccg agcccgagaa gatggcggtg cggaagaagg acggcggccc |
| 121 | caacgtgaag tactacgagg ccgcggacac cgtgacccag ttcgacaacg tgcggctctg |
| 181 | gctcggcaag aactacaaga agtacataca agcagaaccg ccaaccaaca agtctctgtc |
| 241 | cagcctggtg gtgcagttgc tccagtttca ggaagaggtt tttggcaaac atgtcagcaa |
| 301 | cgcaccgctt actaaactgc cgatcaaatg tttcctagat ttcaaagcag gaggatccct |
| 361 | ctgccatatt cttgcagctg cctacaaatt caagagtgac cagggatggc ggcgttacga |
| 421 | tttccagaat ccatcacgca tggaccgcaa tgtggaaatg ttcatgacca ttgagaagtc |
| 481 | cttggtacag aataattgcc tgtcacgacc taacattttc ctctgcccag aaattgagcc |
| 541 | caaactgcta gggaaattaa aagacattgt taagagacac cagggaacca tctctgagga |
| 601 | taagagcaat gcctcccatg ttgtgtatcc tgtcccaggg aacctagaag aagaggaatg |
| 661 | ggtacggcca gtcatgaaga gggataaaca ggttcttctg cactggggct actatcctga |
| 721 | cagctacgac acgtggatcc cagcgagtga aattgaagca tctgtggagg acgctcccac |
| 781 | tcctgagaaa ccgaggaagg tccatgcgaa gtggatcctc gacaccgaca cattcaacga |
| 841 | gtggatgaat gaggaagact acgaagtcag tgacgacaaa agcccagtct cccgcaggaa |
| 901 | gaagatctca gccaagacgc tgacagacga ggtaaacagc ccagattcag acagacgaga |
| 961 | caagaagggg ggcaactata agaagaggaa gcgctctccc tctccttcac ccaccccaga |
| 1021 | ggctaagaag aaaaacgcta agaaaggacc ctcaacacct tataccaagt caaagcgagg |
| 1081 | ccacagagaa gaggaacaag aagacctgac aaaagacatg gatgagccct ctccagtccc |
| 1141 | aaacgtggaa gaggtgacac tccccaaaac agtcaacact aaaaaggact ctgagtcagc |
| 1201 | cccagtcaaa ggcggcacca tgactgacct ggatgaacag gacgatgaaa gcatggagac |
| 1261 | caccggcaag gacgaggatg agaacagcac gggcaacaaa ggcgagcaga cgaagaaccc |
| 1321 | ggacctgcat gaggacaatg tgaccgagca gacccaccac atcatcatcc ccagctacgc |
| 1381 | cgcctggttt gactacaaca gcgtccatgc cattgaacgg agggctcttc ctgagttctt |
| 1441 | caacggcaag aacaagtcta agactccaga gatctacctg gcgtatcgga acttcatgat |
| 1501 | tgacacttac cgactgaatc cccaggagta tctaacatct actgcctgtc ggcggaattt |
| 1561 | ggcgggtgat gtctgcgcta tcatgagggt ccatgccttc ctggaacagt ggggtcttat |
| 1621 | taactaccag gtagatgctg agagccgacc aaccccaatg gggcctccac ccacctctca |
| 1681 | cttccatgtc ttggcggaca caccatcagg gctggttcct cttcagccga agcctccaca |
| 1741 | gcagagctct gcttcccagc aaatgctgaa cttccctgag aagggcaagg agaaaccagc |
| 1801 | agacatgcag aattttgggc tgcgcacaga catgtacaca aagaagaacg tcccctccaa |
| 1861 | gagcaaagct gcagcaagtg ccactcggga atggacggag caggagactc tgctgctcct |
| 1921 | ggaggctttg gaaatgtaca aggacgactg gaacaaagta tctgagcacg tgggaagccg |
| 1981 | cacgcaggac gagtgcatct tgcattttct ccgccttccc attgaagacc catacctgga |
| 2041 | ggactcggag gcttctctag gccctctggc ctaccaaccc atccccttca gtcagtcagg |
| 2101 | caaccctgtt atgagcaccg ttgccttcct ggcctctgtc gtcgatcccc gagttgcctc |
| 2161 | tgctgctgcg aagtcagccc tagaagagtt ctcaaaaatg aaggaagagg tgcccacagc |
| 2221 | tttggtggaa gcccacgtgc gtaaggtcga agaagcggcc aaagtcacag gcaaggccga |
| 2281 | cccagccttt ggtctggaga gtagcggcat cgcagggact gcctctgatg agcctgagcg |
| 2341 | cattgaggaa agcgggactg aggaggcacg gccagagggc caggcagcag atgagaagaa |
| 2401 | ggagcctaag gaaccacggg aaggaggggg cgctgtggag gaagaagcaa aggaggaaat |
| 2461 | aagtgaggtc cccaagaaag atgaagagaa agggaaagaa ggtgacagtg agaaggagtc |
| 2521 | tgagaagagt gacggggacc cgatagttga tcctgagaaa gacaaggaac caacagaagg |
| 2581 | gcaggaggaa gtgctaaagg aagtggcaga gccagagggg gagaggaaaa ccaaggtgga |
| 2641 | gcgtgacatt ggtgaaggca acctgtccac agctgcagcc gcagccctgg ccgctgctgc |
| 2701 | agtcaaggcc aagcacttgg ctgcagttga ggagagaaag atcaagtctt tggtggctct |
| 2761 | gctggtagag acccaaatga agaaactaga gatcaaactc cgacattttg aggagctgga |
| 2821 | gacaataatg gaccgggagc gagaggcgct ggaataccag aggcagcagc tcctggccga |
| 2881 | ccggcaagcc ttccacatgg agcagctgaa gtatgcagag atgagggccc ggcagcagca |
| 2941 | cttccagcag atgcaccagc agcagcagca gcagccacca accttgcccc caggctccca |
| 3001 | gcccatacct cccaccgggg ctgctggacc acctacagtc catggtctag ctgtgcctcc |
| 3061 | agccgctgtg gcctctgccc ctcctggcag tggggcccct cctggaagct tgggcccttc |
| 3121 | tgaacagatt gggcaggcag ggacaactgc agggccacag cagccacaac aagctggagc |
| 3181 | ccctcagcct ggggcagtcc caccaggggt acccccccct ggaccccatg gcccctcacc |
| 3241 | gttccccaac caaccaactc ctccctcaat gatgccaggg gcagtgccag gcagcgggca |
| 3301 | cccaggcgtg gcgggtaatg ctcctttggg tttgcctttt ggcatgccgc ctcctcctcc |
| 3361 | tgctgctcca tccgtcatcc cattcggtag tctagctgac tccattagta ttaaccttcc |
| 3421 | ccctcctcct aacctgcatg ggcatcacca ccatctcccg tttgccccgg gcactatccc |
| 3481 | cccacctaac ctgcctgtgt ccatggcgaa ccctctacat cctaacctgc cggcgaccac |
| 3541 | caccatgcca tcttccttgc ctctcgggcc ggggctcgga tccgccgcag cccagagccc |
| 3601 | tgccattgtg gcagctgttc agggcaacct cctgcccagt gccagcccac tgccagaccc |
| 3661 | aggcaccccg ctgcctccag accccacagc tccaagccca ggcacagtca cccctgtgcc |
| 3721 | acctccacag tgaggaacca gccagccatc tctccccctc actccccatg gagatcacag |
| 3781 | ttccaggaac agccctcccc cactactggg accctccctc agcctgaaga gttcatcact |
| 3841 | acgtaaggaa agctcctcct gccccctcac cacccccacc atgcccagca gaggtgtgca |
| 3901 | gttttatatc caattattat ccacggactt ctgactaaaa gatgtttcta atgcctggga |
| 3961 | gagagaatag gagggaaaga tgtttatacg aggttctact aactggttct gagggtctac |
| 4021 | cccttcagaa ttactgcatt tttgaagtga taacatgaaa atgaaaccct ttaaaaggga |
| 4081 | ggttttaaaa aaagacactt cggagcccac aaaaaaagaa cttttttaat tattattatt |
| 4141 | attattttga ggggaaaggg caggttttaa gaggaattaa atttctgggg caaggtgtga |
| 4201 | ggtggaatag ggcaccgagc ctgtctccct gagcccttgg cagtgctgag tcagctcccc |
| 4261 | tcacccattc cagtttattc atacaaatcc ctcctgctgc tcgtcatggt tgctgtttta |
| 4321 | ggcccagttc agccaatgac cttttcctcc agtcagcttt gtgtttgtgt ttaagtcacc |
| 4381 | tgcttactcg tcagcgtctg tgtacttgtg ggaaatgtag ttttcgggga ttctgtggta |
| 4441 | ggaaatagag gaagaagggg cctcagttgg gctcttcttc ctgctttcct agttgtatct |
| 4501 | gtgagtgccc aacaggcatc agagggggag ctctaagagg atggggggcc tgcagaccct |
| 4561 | caagtttgaa aagcacttaa gcacctactt ttgacagtgg gacagtctgc taacttctgc |
| 4621 | ccccaccaac caagcctgac agaacccagt gatagctagg agttccccaa atgaggacaa |
| 4681 | agatttggga gcagtgcagc gtgcctctgc actccaggtc ttcctcttca ccccctactt |
| 4741 | ggaggcagac acaattccag gccgcaccag agcctggccc ctcccaccag gcgctttgct |
| 4801 | ccttctgtcc cagcgtctcc ttcctctgca tctccacacc tttcttctgt tcaaagtctt |
| 4861 | ctgtaaaatt ttctttcctt ctttgttctt ttctttttcc tttttttttt ataaattaat |
| 4921 | ttgctttcag ttccaaaaaa aaaaaaaaaa aaaaaaaaaa aaaa |

SEQ ID NO: 73 Mouse SMARCC2 Amino Acid Sequence Isoform 1
(NP_001107569.1)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdiv krhqgtised ksnashvvyp |
| 181 | vpgnleeeew vrpvmkrdkq vllhwgyypd sydtwipase ieasvedapt pekprkvhak |
| 241 | wildtdtfne wmneedyevs ddkspvsrrk kisaktltde vnspdsdrrd kkggnykkrk |
| 301 | rspspsptpe akkknakkgp stpytkskrg hreeeqedlt kdmdepspvp nveevtlpkt |
| 361 | vntkkdsesa pvkggtmtdl deqddesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpkppq qssasqqmln fpekgkekpa dmqnfglrtd mytkknvpsk skaaasatre |
| 601 | wteqetllll ealemykddw nkvsehvgsr tqdecilhfl rlpiedpyle dseaslgpla |
| 661 | yqpipfsqsg npvmstvafl asvvdprvas aaaksaleef skmkeevpta lveahvrkve |
| 721 | eaakvtgkad pafglessgi agtasdeper ieesgteear pegqaadekk epkepreggg |
| 781 | aveeeakeei sevpkkdeek gkegdsekes eksdgdpivd pekdkepteg qeevlkevae |
| 841 | pegerktkve rdigegnlst aaaaalaaaa vkakhlaave erkikslval lvetqmkkle |
| 901 | iklrhfeele timdrereal eyqrqqllad rqafhmeqlk yaemrarqqh fqqmhqqqqq |
| 961 | qpptlppgsq pipptgaagp ptvhglavpp aavasappgs gappgslgps eqigqagtta |
| 1021 | gpqqpqqaga pqpgavppgv pppgphgpsp fpnqptppsm mpgavpgsgh pgvagnaplg |
| 1081 | 1pfgmppppp aapsvipfgs ladsisinlp pppnlhghhh hlpfapgtip ppnlpvsman |
| 1141 | plhpnlpatt tmpsslplgp glgsaaaqsp aivaavqgnl lpsasplpdp gtplppdpta |
| 1201 | pspgtvtpvp ppq |

SEQ ID NO: 74 Mouse SMARCC2 cDNA Sequence Variant 2 (NM_001114096.1,
CDS: 92-3484)

| | |
|---|---|
| 1 | gtggcggcgg gaggcggcgg gaggcgggcg gaggaggagg cgggagctga gctgagcggg |
| 61 | gcgggcggcg gcggggcccg agcccgagaa gatggcggtg cggaagaagg acggcggccc |
| 121 | caacgtgaag tactacgagg ccgcggacac cgtgacccag ttcgacaacg tgcggctctg |
| 181 | gctcggcaag aactacaaga agtacataca agcagaaccg ccaaccaaca agtctctgtc |
| 241 | cagcctggtg gtgcagttgc tccagtttca ggaagaggtt tttggcaaac atgtcagcaa |
| 301 | cgcaccgctt actaaactgc cgatcaaatg tttcctagat ttcaaagcag gaggatccct |
| 361 | ctgccatatt cttgcagctg cctacaaatt caagagtgac cagggatggc ggcgttacga |
| 421 | tttccagaat ccatcacgca tggaccgcaa tgtggaaatg ttcatgacca ttgagaagtc |
| 481 | cttggtacag aataattgcc tgtcacgacc taacattttc ctctgcccag aaattgagcc |
| 541 | caaactgcta gggaaattaa aagacattgt taagagacac cagggaacca tctctgagga |
| 601 | taagagcaat gcctcccatg ttgtgtatcc tgtcccaggg aacctagaag aagaggaatg |
| 661 | ggtacggcca gtcatgaaga gggataaaca ggttcttctg cactggggct actatcctga |
| 721 | cagctacgac acgtggatcc cagcgagtga aattgaagca tctgtggagg acgctcccac |
| 781 | tcctgagaaa ccgaggaagg tccatgcgaa gtggatcctc gacaccgaca cattcaacga |
| 841 | gtggatgaat gaggaagact acgaagtcag tgacgacaaa agcccagtct cccgcaggaa |
| 901 | gaagatctca gccaagacgc tgacagacga ggtaaacagc ccagattcag acagacgaga |
| 961 | caagaagggg ggcaactata agaagaggaa gcgctctccc tctccttcac ccaccccaga |
| 1021 | ggctaagaag aaaaacgcta agaaaggacc ctcaacacct tataccaagt caaagcgagg |
| 1081 | ccacagagaa gaggaacaag aagacctgac aaaagacatg gatgagccct ctccagtccc |
| 1141 | aaacgtggaa gaggtgacac tccccaaaac agtcaacact aaaaaggact ctgagtcagc |
| 1201 | cccagtcaaa ggcggcacca tgactgacct ggatgaacag gacgatgaaa gcatggagac |
| 1261 | caccggcaag gacgaggatg agaacagcac gggcaacaaa ggcgagcaga cgaagaaccc |
| 1321 | ggacctgcat gaggacaatg tgaccgagca gacccaccac atcatcatcc ccagctacgc |
| 1381 | cgcctggttt gactacaaca gcgtccatgc cattgaacgg agggctcttc ctgagttctt |
| 1441 | caacggcaag aacaagtcta agactccaga gatctacctg gcgtatcgga acttcatgat |
| 1501 | tgacacttac cgactgaatc cccaggagta tctaacatct actgcctgtc ggcggaattt |
| 1561 | ggcgggtgat gtctgcgcta tcatgagggt ccatgccttc ctggaacagt ggggtcttat |
| 1621 | taactaccag gtagatgctg agagccgacc aaccccaatg gggcctccac ccacctctca |
| 1681 | cttccatgtc ttggcggaca caccatcagg gctggttcct cttcagccga agcctccaca |
| 1741 | gggccgccag gttgatgctg acaccaaggc tgggcggaag ggcaaagagc tggatgacct |
| 1801 | ggtgccagag acggctaagg gcaagccaga gctgcagagc tctgcttccc agcaaatgct |
| 1861 | gaacttccct gagaagggca aggagaaacc agcagacatg cagaattttg ggctgcgcac |
| 1921 | agacatgtac acaaagaaga acgtcccctc caagagcaaa gctgcagcaa gtgccactcg |
| 1981 | ggaatggacg gagcaggaga ctctgctgct cctggaggct ttggaaatgt acaaggacga |
| 2041 | ctggaacaaa gtatctgagc acgtgggaag ccgcacgcag gacgagtgca tcttgcattt |
| 2101 | tctccgcctt cccattgaag acccatacct ggaggactcg gaggcttctc taggccctct |
| 2161 | ggcctaccaa cccatcccct tcagtcagtc aggcaaccct gttatgagca ccgttgcctt |
| 2221 | cctggcctct gtcgtcgatc cccgagttgc ctctgctgct gcgaagtcag ccctagaaga |
| 2281 | gttctcaaaa atgaaggaag aggtgcccac agctttggtg gaagcccacg tgcgtaaggt |
| 2341 | cgaagaagcg gccaaagtca caggcaaggc cgacccagcc tttggtctgg agagtagcgg |
| 2401 | catcgcaggg actgcctctg atgagcctga gcgcattgag gaaagcggga ctgaggaggc |
| 2461 | acggccagag ggccaggcag cagatgagaa gaaggagcct aaggaaccac gggaaggagg |
| 2521 | gggcgctgtg gaggaagaag caaaggagga aataagtgag gtccccaaga aagatgaaga |
| 2581 | gaaagggaaa gaaggtgaca gtgagaagga gtctgagaag agtgacgggg acccgatagt |
| 2641 | tgatcctgag aaagacaagg aaccaacaga agggcaggag gaagtgctaa aggaagtggc |
| 2701 | agagccagag ggggagagga aaaccaaggt ggagcgtgac attggtgaag gcaacctgtc |
| 2761 | cacagctgca gccgcagccc tggccgctgc tgcagtcaag gccaagcact tggctgcagt |
| 2821 | tgaggagaga aagatcaagt ctttggtggc tctgctggta gagacccaaa tgaagaaact |
| 2881 | agagatcaaa ctccgacatt ttgaggagct ggagacaata atggaccggg agcgagaggc |
| 2941 | gctggaatac cagaggcagc agctcctggc cgaccggcaa gccttccaca tggagcagct |
| 3001 | gaagtatgca gagatgaggg cccggcagca gcacttccag cagatgcacc agcagcagca |
| 3061 | gcagcagcca ccaaccttgc ccccaggctc ccagcccata cctcccaccg gggctgctgg |
| 3121 | accacctaca gtccatggtc tagctgtgcc tccagccgct gtggcctctg cccctcctgg |
| 3181 | cagtggggcc cctcctggaa gcttgggccc ttctgaacag attgggcagg cagggacaac |
| 3241 | tgcagggcca cagcagccac aacaagctgg agcccctcag cctggggcag tcccaccagg |
| 3301 | ggtacccccc cctggacccc atggcccctc accgttcccc aaccaaccaa ctcctccctc |
| 3361 | aatgatgcca ggggcagtgc caggcagcgg gcacccaggc gtggcggacc caggcacccc |
| 3421 | gctgcctcca gaccccacag ctccaagccc aggcacagtc acccctgtgc cacctccaca |
| 3481 | gtgaggaacc agccagccat ctctccccct cactccccat ggagatcaca gttccaggaa |
| 3541 | cagccctccc ccactactgg gaccctccct cagcctgaag agttcatcac tacgtaagga |
| 3601 | aagctcctcc tgccccctca ccacccccac catgcccagc agaggtgtgc agttttatat |
| 3661 | ccaattatta tccacggact tctgactaaa agatgtttct aatgcctggg agagagaata |
| 3721 | ggagggaaag atgtttatac gaggttctac taactggttc tgagggtcta ccccttcaga |
| 3781 | attactgcat ttttgaagtg ataacatgaa aatgaaaccc tttaaaaggg aggttttaaa |
| 3841 | aaaagacact tcggagccca caaaaaaaga acttttttaa ttattattat tattattttg |
| 3901 | aggggaaagg gcaggtttta agaggaatta aatttctggg gcaaggtgtg aggtggaata |
| 3961 | gggcaccgag cctgtctccc tgagcccttg gcagtgctga gtcagctccc ctcacccatt |
| 4021 | ccagtttatt catacaaatc cctcctgctg ctcgtcatgg ttgctgtttt aggcccagtt |
| 4081 | cagccaatga ccttttcctc cagtcagctt tgtgtttgtg tttaagtcac ctgcttactc |
| 4141 | gtcagcgtct gtgtacttgt gggaaatgta gttttcgggg attctgtggt aggaaataga |
| 4201 | ggaagaaggg gcctcagttg ggctcttctt cctgctttcc tagttgtatc tgtgagtgcc |
| 4261 | caacaggcat cagaggggga gctctaagag gatggggggc ctgcagaccc tcaagtttga |
| 4321 | aaagcactta agcacctact tttgacagtg ggacagtctg ctaacttctg cccccaccaa |
| 4381 | ccaagcctga cagaacccag tgatagctag gagttcccca aatgaggaca aagatttggg |
| 4441 | agcagtgcag cgtgcctctg cactccaggt cttcctcttc accccctact tggaggcaga |
| 4501 | cacaattcca ggccgcacca gagcctggcc cctcccacca ggcgctttgc tccttctgtc |
| 4561 | ccagcgtctc cttcctctgc atctccacac ctttcttctg ttcaaagtct tctgtaaaat |
| 4621 | tttctttcct tctttgttct tttctttttc cttttttttt tataaattaa tttgctttca |
| 4681 | gttccaaaaa aaaaaaaaaa aaaaaaaaaa aaaaa |

SEQ ID NO: 75 Mouse SMARCC2 Amino Acid Sequence Isoform 2
(NP_001107568.1)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdiv krhqgtised ksnashvvyp |
| 181 | vpgnleeeew vrpvmkrdkq vllhwgyypd sydtwipase ieasvedapt pekprkvhak |
| 241 | wildtdtfne wmneedyevs ddkspvsrrk kisaktltde vnspdsdrrd kkggnykkrk |
| 301 | rspspsptpe akkknakkgp stpytkskrg hreeeqedlt kdmdepspvp nveevtlpkt |
| 361 | vntkkdsesa pvkggtmtdl deqddesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpkppq grqvdadtka grkgkelddl vpetakgkpe lqssasqqml nfpekgkekp |
| 601 | admqnfglrt dmytkknvps kskaaasatr ewteqetlll lealemykdd wnkvsehvgs |
| 661 | rtqdecilhf lrlpiedpyl edseaslgpl ayqpipfsqs gnpvmstvaf lasvvdprva |
| 721 | saaaksalee fskmkeevpt alveahvrkv eeaakvtgka dpafglessg iagtasdepe |
| 781 | rieesgteea rpegqaadek kepkepregg gaveeeakee isevpkkdee kgkegdseke |
| 841 | seksdgdpiv dpekdkepte gqeevlkeva epegerktkv erdigegnls taaaaalaaa |
| 901 | avkakhlaav eerkikslva llvetqmkkl eiklrhfeel etimdrerea leyqrqqlla |
| 961 | drqafhmeql kyaemrarqq hfqqmhqqqq qqpptlppgs qpipptgaag pptvhglavp |
| 1021 | paavasappg sgappgslgp seqigqagtt agpqqpqqag apqpgavppg vpppgphgps |
| 1081 | pfpnqptpps mmpgavpgsg hpgvadpgtp lppdptapsp gtvtpvpppq |

SEQ ID NO: 76 Mouse SMARCC2 cDNA Sequence Variant 3 (NM_198160.2, CDS:
92-3391)

| | |
|---|---|
| 1 | gtggcggcgg gaggcggcgg gaggcgggcg gaggaggagg cgggagctga gctgagcggg |
| 61 | gcgggcggcg gcggggcccg agcccgagaa gatggcggtg cggaagaagg acggcggccc |
| 121 | caacgtgaag tactacgagg ccgcggacac cgtgacccag ttcgacaacg tgcggctctg |
| 181 | gctcggcaag aactacaaga agtacataca agcagaaccg ccaaccaaca agtctctgtc |
| 241 | cagcctggtg gtgcagttgc tccagtttca ggaagaggtt tttggcaaac atgtcagcaa |
| 301 | cgcaccgctt actaaactgc cgatcaaatg tttcctagat ttcaaagcag gaggatccct |
| 361 | ctgccatatt cttgcagctg cctacaaatt caagagtgac cagggatggc ggcgttacga |
| 421 | tttccagaat ccatcacgca tggaccgcaa tgtggaaatg ttcatgacca ttgagaagtc |
| 481 | cttggtacag aataattgcc tgtcacgacc taacattttc ctctgcccag aaattgagcc |
| 541 | caaactgcta gggaaattaa aagacattgt taagagacac cagggaacca tctctgagga |
| 601 | taagagcaat gcctcccatg ttgtgtatcc tgtcccaggg aacctagaag aagaggaatg |
| 661 | ggtacggcca gtcatgaaga gggataaaca ggttcttctg cactggggct actatcctga |
| 721 | cagctacgac acgtggatcc cagcgagtga aattgaagca tctgtggagg acgctcccac |
| 781 | tcctgagaaa ccgaggaagg tccatgcgaa gtggatcctc gacaccgaca cattcaacga |
| 841 | gtggatgaat gaggaagact acgaagtcag tgacgacaaa agcccagtct cccgcaggaa |
| 901 | gaagatctca gccaagacgc tgacagacga ggtaaacagc ccagattcag acagacgaga |
| 961 | caagaagggg ggcaactata agaagaggaa gcgctctccc tctccttcac ccaccccaga |
| 1021 | ggctaagaag aaaaacgcta agaaaggacc ctcaacacct tataccaagt caaagcgagg |
| 1081 | ccacagagaa gaggaacaag aagacctgac aaaagacatg gatgagccct ctccagtccc |
| 1141 | aaacgtggaa gaggtgacac tccccaaaac agtcaacact aaaaaggact ctgagtcagc |
| 1201 | cccagtcaaa ggcggcacca tgactgacct ggatgaacag gacgatgaaa gcatggagac |
| 1261 | caccggcaag gacgaggatg agaacagcac gggcaacaaa ggcgagcaga cgaagaaccc |
| 1321 | ggacctgcat gaggacaatg tgaccgagca gacccaccac atcatcatcc ccagctacgc |
| 1381 | cgcctggttt gactacaaca gcgtccatgc cattgaacgg agggctcttc ctgagttctt |
| 1441 | caacggcaag aacaagtcta agactccaga gatctacctg gcgtatcgga acttcatgat |
| 1501 | tgacacttac cgactgaatc cccaggagta tctaacatct actgcctgtc ggcggaattt |
| 1561 | ggcgggtgat gtctgcgcta tcatgagggt ccatgccttc ctggaacagt ggggtcttat |
| 1621 | taactaccag gtagatgctg agagccgacc aaccccaatg gggcctccac ccacctctca |
| 1681 | cttccatgtc ttggcggaca caccatcagg gctggttcct cttcagccga agcctccaca |
| 1741 | gcagagctct gcttcccagc aaatgctgaa cttccctgag aagggcaagg agaaaccagc |
| 1801 | agacatgcag aattttgggc tgcgcacaga catgtacaca aagaagaacg tcccctccaa |
| 1861 | gagcaaagct gcagcaagtg ccactcggga atggacggag caggagactc tgctgctcct |
| 1921 | ggaggctttg gaaatgtaca aggacgactg gaacaaagta tctgagcacg tgggaagccg |
| 1981 | cacgcaggac gagtgcatct tgcattttct ccgccttccc attgaagacc catacctgga |
| 2041 | ggactcggag gcttctctag gccctctggc ctaccaaccc atccccttca gtcagtcagg |
| 2101 | caaccctgtt atgagcaccg ttgccttcct ggcctctgtc gtcgatcccc gagttgcctc |
| 2161 | tgctgctgcg aagtcagccc tagaagagtt ctcaaaaatg aaggaagagg tgcccacagc |
| 2221 | tttggtggaa gcccacgtgc gtaaggtcga agaagcggcc aaagtcacag gcaaggccga |
| 2281 | cccagccttt ggtctggaga gtagcggcat cgcagggact gcctctgatg agcctgagcg |
| 2341 | cattgaggaa agcgggactg aggaggcacg gccagagggc caggcagcag atgagaagaa |
| 2401 | ggagcctaag gaaccacggg aaggaggggg cgctgtggag gaagaagcaa aggaggaaat |
| 2461 | aagtgaggtc cccaagaaag atgaagagaa agggaaagaa ggtgacagtg agaaggagtc |
| 2521 | tgagaagagt gacggggacc cgatagttga tcctgagaaa gacaaggaac caacagaagg |
| 2581 | gcaggaggaa gtgctaaagg aagtggcaga gccagagggg gagaggaaaa ccaaggtgga |
| 2641 | gcgtgacatt ggtgaaggca acctgtccac agctgcagcc gcagccctgg ccgctgctgc |
| 2701 | agtcaaggcc aagcacttgg ctgcagttga ggagagaaag atcaagtctt tggtggctct |
| 2761 | gctggtagag acccaaatga agaaactaga gatcaaactc cgacattttg aggagctgga |
| 2821 | gacaataatg gaccgggagc gagaggcgct ggaataccag aggcagcagc tcctggccga |
| 2881 | ccggcaagcc ttccacatgg agcagctgaa gtatgcagag atgagggccc ggcagcagca |
| 2941 | cttccagcag atgcaccagc agcagcagca gcagccacca accttgcccc caggctccca |
| 3001 | gcccatacct cccaccgggg ctgctggacc acctacagtc catggtctag ctgtgcctcc |
| 3061 | agccgctgtg gcctctgccc ctcctggcag tggggcccct cctggaagct tgggcccttc |
| 3121 | tgaacagatt gggcaggcag ggacaactgc agggccacag cagccacaac aagctggagc |
| 3181 | ccctcagcct ggggcagtcc caccaggggt acccccccct ggaccccatg gcccctcacc |
| 3241 | gttccccaac caaccaactc ctccctcaat gatgccaggg gcagtgccag gcagcgggca |
| 3301 | cccaggcgtg gcggacccag gcaccccgct gcctccagac cccacagctc caagcccagg |
| 3361 | cacagtcacc cctgtgccac ctccacagtg aggaaccagc cagccatctc tccccctcac |
| 3421 | tccccatgga gatcacagtt ccaggaacag ccctccccca ctactgggac cctccctcag |
| 3481 | cctgaagagt tcatcactac gtaaggaaag ctcctcctgc cccctcacca cccccaccat |
| 3541 | gcccagcaga ggtgtgcagt tttatatcca attattatcc acggacttct gactaaaaga |
| 3601 | tgtttctaat gcctgggaga gagaatagga gggaaagatg tttatacgag gttctactaa |
| 3661 | ctggttctga gggtctaccc cttcagaatt actgcatttt tgaagtgata acatgaaaat |
| 3721 | gaaacccttt aaaagggagg ttttaaaaaa agacacttcg gagcccacaa aaaaagaact |
| 3781 | tttttaatta ttattattat tattttgagg ggaaagggca ggttttaaga ggaattaaat |
| 3841 | ttctggggca aggtgtgagg tggaataggg caccgagcct gtctccctga gcccttggca |
| 3901 | gtgctgagtc agctcccctc acccattcca gtttattcat acaaatccct cctgctgctc |
| 3961 | gtcatggttg ctgttttagg cccagttcag ccaatgacct tttcctccag tcagctttgt |
| 4021 | gtttgtgttt aagtcacctg cttactcgtc agcgtctgtg tacttgtggg aaatgtagtt |
| 4081 | ttcggggatt ctgtggtagg aaatagagga agaaggggcc tcagttgggc tcttcttcct |
| 4141 | gctttcctag ttgtatctgt gagtgcccaa caggcatcag agggggagct ctaagaggat |
| 4201 | ggggggcctg cagaccctca agtttgaaaa gcacttaagc acctactttt gacagtggga |
| 4261 | cagtctgcta acttctgccc ccaccaacca agcctgacag aacccagtga tagctaggag |
| 4321 | ttccccaaat gaggacaaag atttgggagc agtgcagcgt gcctctgcac tccaggtctt |
| 4381 | cctcttcacc ccctacttgg aggcagacac aattccaggc cgcaccagag cctggcccct |
| 4441 | cccaccaggc gctttgctcc ttctgtccca gcgtctcctt cctctgcatc tccacacctt |
| 4501 | tcttctgttc aaagtcttct gtaaaatttt ctttccttct ttgttctttt ctttttcctt |
| 4561 | ttttttttat aaattaattt gctttcagtt ccaaaaaaaa aaaaaaaaaa aaaaaaaaaa |
| 4621 | aa |

SEQ ID NO: 77 Mouse SMARCC2 Amino Acid Sequence Isoform 3 (NP_937803.1)

| | |
|---|---|
| 1 | mavrkkdggp nvkyyeaadt vtqfdnvrlw lgknykkyiq aepptnksls slvvqllqfq |
| 61 | eevfgkhvsn apltklpikc fldfkaggsl chilaaaykf ksdqgwrryd fqnpsrmdrn |
| 121 | vemfmtieks lvqnnclsrp niflcpeiep kllgklkdiv krhqgtised ksnashvvyp |
| 181 | vpgnleeeew vrpvmkrdkq vllhwgyypd sydtwipase ieasvedapt pekprkvhak |
| 241 | wildtdtfne wmneedyevs ddkspvsrrk kisaktltde vnspdsdrrd kkggnykkrk |
| 301 | rspspsptpe akkknakkgp stpytkskrg hreeeqedlt kdmdepspvp nveevtlpkt |
| 361 | vntkkdsesa pvkggtmtdl deqddesmet tgkdedenst gnkgeqtknp dlhednvteq |
| 421 | thhiiipsya awfdynsvha ierralpeff ngknksktpe iylayrnfmi dtyrlnpqey |
| 481 | ltstacrrnl agdvcaimrv hafleqwgli nyqvdaesrp tpmgppptsh fhvladtpsg |
| 541 | lvplqpkppq qssasqqmln fpekgkekpa dmqnfglrtd mytkknvpsk skaaasatre |
| 601 | wteqetllll ealemykddw nkvsehvgsr tqdecilhfl rlpiedpyle dseaslgpla |
| 661 | yqpipfsqsg npvmstvafl asvvdprvas aaaksaleef skmkeevpta lveahvrkve |
| 721 | eaakvtgkad pafglessgi agtasdeper ieesgteear pegqaadekk epkepreggg |
| 781 | aveeeakeei sevpkkdeek gkegdsekes eksdgdpivd pekdkepteg qeevlkevae |
| 841 | pegerktkve rdigegnlst aaaaalaaaa vkakhlaave erkikslval lvetqmkkle |
| 901 | iklrhfeele timdrereal eyqrqqllad rqafhmeqlk yaemrarggh fqqmhqqqqq |
| 961 | qpptlppgsq pipptgaagp ptvhglavpp aavasappgs gappgslgps eqigqagtta |
| 1021 | gpqqpqqaga pqpgavppgv pppgphgpsp fpnqptppsm mpgavpgsgh pgvadpgtpl |
| 1081 | ppdptapspg tvtpvpppq |

SEQ ID NO: 78 Human SMARCD1 cDNA Sequence Variant 1 (NM_003076.4,
CDS: 171-1718)

| | |
|---|---|
| 1 | agcacgcctt ttccgctagt cgccccgctc tatcccatag tctcgctgcc ctgagcctcc |
| 61 | cgtgccggcc ggccggccgg gggaacaggc gggcgctcgg ggggcgctcg gggggcgggg |
| 121 | ggagttccgg ttccggttct ttgtgcggct gcatcggcgg ctccgggaag atggcggccc |
| 181 | gggcgggttt ccagtctgtg gctccaagcg gcggcgccgg agcctcagga ggggcgggcg |
| 241 | cggctgctgc cttgggcccg ggcggaactc cggggcctcc tgtgcgaatg ggcccggctc |
| 301 | cgggtcaagg gctgtaccgc tccccgatgc ccggagcggc ctatccgaga ccaggtatgt |
| 361 | tgccaggcag ccgaatgaca cctcagggac cttccatggg accccctggc tatgggggga |
| 421 | acccttcagt ccgacctggc ctggcccagt cagggatgga tcagtcccgc aagagacctg |
| 481 | cccctcagca gatccagcag gtccagcagc aggcggtcca aaatcgaaac cacaatgcaa |
| 541 | agaaaaagaa gatggctgac aaaattctac ctcaaaggat tcgtgaactg gtaccagaat |
| 601 | cccaggccta tatggatctc ttggcttttg aaaggaaact ggaccagact atcatgagga |
| 661 | aacggctaga tatccaagag gccttgaaac gtcccatcaa gcaaaaacgg aagctgcgaa |
| 721 | ttttcatttc taacactttc aatccggcta agtcagatgc cgaggatggg gaagggacgg |
| 781 | tggcttcctg ggagcttcgg gtagaaggac ggctcctgga ggattcagcc ttgtccaaat |
| 841 | atgatgccac taaacaaaag aggaagttct cttccttttt taagtccttg gtgattgaac |
| 901 | tggacaaaga cctgtatggg ccagacaacc atctggtaga atggcacagg accgccacta |
| 961 | cccaggagac cgatggcttt caggtgaagc ggccgggaga cgtgaatgta cggtgtactg |
| 1021 | tcctactgat gctggattac cagcctcccc agtttaaatt agacccccgc ctagctcgac |
| 1081 | tcctgggcat ccatacccag actcgtccag tgatcatcca agcactgtgg caatatatta |
| 1141 | agacacataa gctccaggac cctcacgagc gggagtttgt catctgtgac aagtacctgc |
| 1201 | agcagatctt tgagtctcaa cgtatgaagt tttcagagat ccctcagcgg ctccatgcct |
| 1261 | tgcttatgcc accagaacct atcatcatta atcatgtcat cagtgttgac ccgaatgatc |
| 1321 | agaaaaagac agcttgttat gacattgatg ttgaagtgga tgacaccttg aagacccaga |
| 1381 | tgaattcttt tctgctgtcc actgccagcc aacaggagat tgctactcta gacaacaaga |
| 1441 | tccatgagac aatagaaacc atcaaccagc tgaagactca gcgggagttc atgctgagct |
| 1501 | ttgccagaga ccctcagggt ttcatcaatg actggcttca gtcccagtgc agggacctca |
| 1561 | agacaatgac tgatgtggtg ggtaacccag aggaggagcg ccgagctgag ttctacttcc |
| 1621 | agccctgggc tcaggaggct gtgtgccgat acttctactc caaggtgcag cagagacgac |
| 1681 | aagaattaga gcaagccctg ggaatccgga atacataggg cctctcccac agccctgatt |
| 1741 | cgactgcacc aattcttgat ttgggccctg tgctgcctgc ctcatagtat ctgccttggt |
| 1801 | cttgcttggg gcgttccagg ggatgctgtt ggttcaagga caacaccaga atgaagaggg |
| 1861 | tctcacaaga cacctgttat cctcttcttt caccctatct cttcccaccc ccagcttccc |
| 1921 | tttgccccac aaagttccca tgtgcctgta ccctcccctg gtctacatag gacctctaga |
| 1981 | tagtgttaga gagagaacat gtagtggtaa tgagtgcttg gaatggattg ggcctcaggc |
| 2041 | caggtggtct tcaaggggac cagctaactg atcctgccct tcagagaccc aggagttggg |
| 2101 | agctttcgct ccttctccaa gactcaggcc tgtgggcact ctataagcta gttgatcttg |
| 2161 | gctctcctga taacagaatc caatttcctt ccttccctcc acaggtttgg aacaaactct |
| 2221 | cccttcactt gttgccctgt agcactacag aaaccctggt tcttgggctc cactgagccc |
| 2281 | caggtcagtc cccagccctc tgggttggcc tgctgtcagt gcttctctca ctccttagtt |
| 2341 | ggggtccaca tcagtattgg agttttgttc tttattgctc cctcccagac actccctgtg |
| 2401 | gctgcccttt gtgattccct cagatctgcc ctaatcccgg gcatttgggt gggggaatct |
| 2461 | tgcctttccc tttcagagcc ccagggatct catctgggga actgtcattg ccagcagagg |
| 2521 | ctgttccttc ctgctgtttg gagatgtgac tcattcattc actcactcca ccctgcctct |
| 2581 | gcatccctta atggagaaac gggcctaaaa ccaaacgggt aaaaagccct gggccatccc |
| 2641 | tgtcttcctg tcccttgtct gcccagttga cacctactgg tgacttctag ggcactgagg |
| 2701 | agtgaaagcg cctagggctg gagaatagcg ctgagttggg tttgtgactc ttccctctcc |
| 2761 | ctgcctcaca ggattgtgac tccccagccc ctgccctcaa agcttcagac ccctcaggta |
| 2821 | gcagcaggac cttgtgatct tggccccttg gatctgagat ggtttttgca tctttccagg |
| 2881 | agagcctcac attcttcttc caggttgtat cacccccgag ttagcatatc ccaggctcgc |
| 2941 | agactcaaca cagcaagggt gggagacagc tgggcacaaa gggggaattc cgttcagcat |
| 3001 | gggctctaaa cccacagaac tgacaaagcc cctgcttccc caccccctcc tcaggctcct |
| 3061 | gcgagcacac ccccaccccc aaatccctcc ctgttctaca ctggggacag cagaattttc |
| 3121 | tccccgtctt ccccttcctg ccattttccc tcccttgaaa ggttgacact ggacaacctt |
| 3181 | ggggcagctg agccctggcc gcctcctggc tggaaccatg agaaggaagc tcagtacttc |
| 3241 | ccacagtgtc cctgttgata actgttttta ttaactgaat tgtttttttc atggaccaaa |
| 3301 | cttttttttg tactgtcccc ttattgatgt tacccagttt taataaaaga atcttctgaa |
| 3361 | ggatgggtcc tcctacctac tgtgagagag ctcttccctg agctcttctt ccttcaatac |
| 3421 | cattagccaa a |

SEQ ID NO: 79 Human SMARCD1 Amino Acid Sequence Isoform A
(NP_003067.3)

| | |
|---|---|
| 1 | maaragfqsv apsggagasg gagaaaalgp ggtpgppvrm gpapgqglyr spmpgaaypr |
| 61 | pgmlpgsrmt pqgpsmgppg yggnpsvrpg laqsgmdqsr krpapqqiqq vqqqavqnrn |
| 121 | hnakkkkmad kilpqrirel vpesqaymdl laferkldqt imrkrldiqe alkrpikqkr |
| 181 | klrifisntf npaksdaedg egtvaswelr vegrlledsa lskydatkqk rkfssffksl |
| 241 | vieldkdlyg pdnhlvewhr tattqetdgf qvkrpgdvnv rctvllmldy qppqfkldpr |
| 301 | larllgihtq trpviiqalw qyikthklqd pherefvicd kylqqifesq rmkfseipqr |
| 361 | lhallmppep iiinhvisvd pndqkktacy didvevddtl ktqmnsflls tasqqeiatl |
| 421 | dnkihetiet inqlktqref mlsfardpqg findwlqsqc rdlktmtdvv gnpeeerrae |
| 481 | fyfqpwaqea vcryfyskvq qrrqeleqal girnt |

SEQ ID NO: 80 Human SMARCD1 cDNA Sequence Variant 2 (NM 139071.2,
CDS: 171-1595)

| | |
|---|---|
| 1 | agcacgcctt ttccgctagt cgccccgctc tatcccatag tctcgctgcc ctgagcctcc |
| 61 | cgtgccggcc ggccggccgg gggaacaggc gggcgctcgg ggggcgctcg gggggcgggg |
| 121 | ggagttccgg ttccggttct ttgtgcggct gcatcggcgg ctccgggaag atggcggccc |
| 181 | gggcgggttt ccagtctgtg gctccaagcg gcggcgccgg agcctcagga ggggcgggcg |
| 241 | cggctgctgc cttgggcccg ggcggaactc cggggcctcc tgtgcgaatg ggcccggctc |
| 301 | cgggtcaagg gctgtaccgc tccccgatgc ccggagcggc ctatccgaga ccaggtatgt |
| 361 | tgccaggcag ccgaatgaca cctcagggac cttccatggg accccctggc tatgggggga |
| 421 | acccttcagt ccgacctggc ctggcccagt cagggatgga tcagtcccgc aagagacctg |
| 481 | cccctcagca gatccagcag gtccagcagc aggcggtcca aaatcgaaac cacaatgcaa |
| 541 | agaaaaagaa gatggctgac aaaattctac ctcaaaggat tcgtgaactg gtaccagaat |
| 601 | cccaggccta tatggatctc ttggcttttg aaaggaaact ggaccagact atcatgagga |
| 661 | aacggctaga tatccaagag gccttgaaac gtcccatcaa gcaaaaacgg aagctgcgaa |
| 721 | ttttcatttc taacactttc aatccggcta agtcagatgc cgaggatggg gaagggacgg |
| 781 | tggcttcctg ggagcttcgg gtagaaggac ggctcctgga ggattcagcc ttgtccaaat |
| 841 | atgatgccac taaacaaaag aggaagttct cttccttttt taagtccttg gtgattgaac |
| 901 | tggacaaaga cctgtatggg ccagacaacc atctggtaga atggcacagg accgccacta |
| 961 | cccaggagac cgatggcttt caggtgaagc ggccgggaga cgtgaatgta cggtgtactg |
| 1021 | tcctactgat gctggattac cagcctcccc agtttaaatt agacccccgc ctagctcgac |
| 1081 | tcctgggcat ccatacccag actcgtccag tgatcatcca agcactgtgg caatatatta |
| 1141 | agacacataa gctccaggac cctcacgagc gggagtttgt catctgtgac aagtacctgc |
| 1201 | agcagatctt tgagtctcaa cgtatgaagt tttcagagat ccctcagcgg ctccatgcct |
| 1261 | tgcttatgcc accagaacct atcatcatta atcatgtcat cagtgttgac ccgaatgatc |
| 1321 | agaaaaagac agcttgttat gacattgatg ttgaagtgga tgacaccttg aagacccaga |
| 1381 | tgaattcttt tctgctgtcc actgccagcc aacaggagat tgctactcta gacaacaaga |
| 1441 | caatgactga tgtggtgggt aacccagagg aggagcgccg agctgagttc tacttccagc |
| 1501 | cctgggctca ggaggctgtg tgccgatact tctactccaa ggtgcagcag agacgacaag |
| 1561 | aattagagca agccctggga atccggaata catagggcct ctcccacagc cctgattcga |
| 1621 | ctgcaccaat tcttgatttg ggccctgtgc tgcctgcctc atagtatctg ccttggtctt |
| 1681 | gcttggggcg ttccagggga tgctgttggt tcaaggacaa caccagaatg aagagggtct |
| 1741 | cacaagacac ctgttatcct cttctttcac cctatctctt cccaccccca gcttcccttt |
| 1801 | gccccacaaa gttcccatgt gcctgtaccc tcccctggtc tacataggac ctctagatag |
| 1861 | tgttagagag agaacatgta gtggtaatga gtgcttggaa tggattgggc ctcaggccag |
| 1921 | gtggtcttca aggggaccag ctaactgatc ctgcccttca gagacccagg agttgggagc |
| 1981 | tttcgctcct tctccaagac tcaggcctgt gggcactcta taagctagtt gatcttggct |
| 2041 | ctcctgataa cagaatccaa tttccttcct tccctccaca ggtttggaac aaactctccc |
| 2101 | ttcacttgtt gccctgtagc actacagaaa ccctggttct tgggctccac tgagccccag |
| 2161 | gtcagtcccc agccctctgg gttggcctgc tgtcagtgct tctctcactc cttagttggg |
| 2221 | gtccacatca gtattggagt tttgttcttt attgctccct cccagacact ccctgtggct |
| 2281 | gccctttgtg attccctcag atctgcccta atcccgggca tttgggtggg ggaatcttgc |
| 2341 | ctttcccttt cagagcccca gggatctcat ctggggaact gtcattgcca gcagaggctg |
| 2401 | ttccttcctg ctgtttggag atgtgactca ttcattcact cactccaccc tgcctctgca |
| 2461 | tcccttaatg gagaaacggg cctaaaacca aacgggtaaa aagccctggg ccatccctgt |
| 2521 | cttcctgtcc cttgtctgcc cagttgacac ctactggtga cttctagggc actgaggagt |
| 2581 | gaaagcgcct agggctggag aatagcgctg agttgggttt gtgactcttc cctctccctg |
| 2641 | cctcacagga ttgtgactcc ccagcccctg ccctcaaagc ttcagacccc tcaggtagca |
| 2701 | gcaggacctt gtgatcttgg ccccttggat ctgagatggt ttttgcatct ttccaggaga |
| 2761 | gcctcacatt cttcttccag gttgtatcac ccccgagtta gcatatccca ggctcgcaga |
| 2821 | ctcaacacag caagggtggg agacagctgg gcacaaaggg ggaattccgt tcagcatggg |
| 2881 | ctctaaaccc acagaactga caaagcccct gcttccccac cccctcctca ggctcctgcg |
| 2941 | agcacacccc cacccccaaa tccctccctg ttctacactg gggacagcag aattttctcc |
| 3001 | ccgtcttccc cttcctgcca ttttccctcc cttgaaaggt tgacactgga caaccttggg |
| 3061 | gcagctgagc cctggccgcc tcctggctgg aaccatgaga aggaagctca gtacttccca |
| 3121 | cagtgtccct gttgataact gtttttatta actgaattgt ttttttcatg gaccaaactt |
| 3181 | ttttttgtac tgtcccctta ttgatgttac ccagttttaa taaaagaatc ttctgaagga |
| 3241 | tgggtcctcc tacctactgt gagagagctc ttccctgagc tcttcttcct tcaataccat |
| 3301 | tagccaaa |

SEQ ID NO: 81 Human SMARCD1 Amino Acid Sequence Isoform B
(NP_620710.2)

| | |
|---|---|
| 1 | maaragfqsv apsggagasg gagaaaalgp ggtpgppvrm gpapgqglyr spmpgaaypr |
| 61 | pgmlpgsrmt pqgpsmgppg yggnpsvrpg laqsgmdqsr krpapqqiqq vqqqavqnrn |
| 121 | hnakkkkmad kilpqrirel vpesqaymdl laferkldqt imrkrldiqe alkrpikqkr |
| 181 | klrifisntf npaksdaedg egtvaswelr vegrlledsa lskydatkqk rkfssffksl |
| 241 | vieldkdlyg pdnhlvewhr tattqetdgf qvkrpgdvnv rctvllmldy qppqfkldpr |
| 301 | larllgihtq trpviiqalw qyikthklqd pherefvicd kylqqifesq rmkfseipqr |
| 361 | lhallmppep iiinhvisvd pndqkktacy didvevddtl ktqmnsflls tasqqeiatl |
| 421 | dnktmtdvvg npeeerraef yfqpwaqeav cryfyskvqq rrqeleqalg irnt |

SEQ ID NO: 82 Mouse SMARCD1 cDNA Sequence (NM 031842.2, CDS: 36-1583)

| | |
|---|---|
| 1 | gttctttgtg cagctgcagc ggcggctccg ggaagatggc ggcccgggcg ggtttccagt |
| 61 | ctgtggctcc gagcggcggc gcgggagcct caggaggagc gggcgtggcg gctgctctgg |
| 121 | gcccgggcgg aactcccggg cctcccgtgc gaatgggccc ggcgccgggt caagggctgt |
| 181 | accgctctcc gatgcccggg gcggcctatc cgagaccagg tatgctgcca ggtagccgaa |
| 241 | tgacacctca gggaccttcc atgggacctc ctggctatgg ggggaaccct tcagtccgac |
| 301 | ctggtctggc ccagtcaggg atggaccagt cccgcaagag acctgcacct caacagatcc |
| 361 | agcaggtcca gcagcaggcg gtccaaaatc gaaatcacaa tgcaaagaaa aagaagatgg |
| 421 | ctgacaaaat cctacctcaa aggattcggg aactggtccc agaatcacag gcctacatgg |
| 481 | atctcctggc ttttgaaagg aaactggacc agactattat gaggaagcgg ctagatatcc |
| 541 | aggaggcctt gaaacgtccc atcaagcaaa aacggaagct gcgaattttc atttctaaca |
| 601 | cgttcaatcc ggctaagtcg gacgcggagg atggggaagg gacggtggct tcctgggagc |
| 661 | tccgggtaga aggccggctc ctggaggacg cggccttgtc caaatatgac gccaccaagc |
| 721 | aaaagagaaa gttctcttcc ttttttaagt ccttggtgat cgaactggac aaagacctct |
| 781 | atggcccaga caaccatctg gtagaatggc acaggaccgc cactacccag gagaccgatg |
| 841 | gcttccaggt gaagcggcca ggagatgtga atgtacggtg tactgtcctg ctgatgctgg |
| 901 | actaccagcc cccccagttt aaattagacc ctcgcctggc tcggctcttg ggcatccata |
| 961 | cccagacacg tccagtgatc atccaagcac tgtggcagta tattaaaaca cacaagctcc |
| 1021 | aggaccctca cgagcgagag tttgttctct gtgacaagta cctccagcag atctttgaat |
| 1081 | ctcagcggat gaagttctca gagatccctc agcggctcca cgccttgctt atgccaccag |
| 1141 | agcccatcat catcaatcat gtcatcagtg tggacccaaa tgaccagaaa aagaccgcgt |
| 1201 | gctatgacat tgacgtggag gtggatgaca ctctgaagac ccagatgaac tctttcctgt |
| 1261 | tgtccactgc cagccagcag gagatcgcca ctctagacaa caagatccat gagacgatag |
| 1321 | agaccatcaa ccagctgaag acccagcgag agttcatgtt gagctttgcc cgagaccctc |
| 1381 | agggtttcat caatgattgg cttcagtccc agtgcaggga cctcaagacg atgactgatg |
| 1441 | tggtgggtaa cccggaagag gagcgtcgtg ctgagttcta cttccagccc tgggctcagg |
| 1501 | aggctgtgtg ccgatacttc tactccaagg tgcagcagag gcggcaagag ttagagcaag |
| 1561 | ccctgggaat ccgaaacaca tagggcctct gtggccctag cctggctgca ccgattcctt |
| 1621 | gggccctgtg ctgcctgcct cagtgtacct gtcttggtct tgcttgaggc attccagggg |
| 1681 | acttggcttc aggacagtgt cacaatgaag agggtgtcac atttctgtct cacagtcacc |
| 1741 | tgttatcccg tcctgtaccc cagtcgtccc ccgtcccgtc gtgtcccccc ctcaccccac |
| 1801 | cccgcctcag ctcctcccca tcaggctcct gtgtgcctct acctccctat cctacatagg |
| 1861 | acctctagat agtgttagag aaccacagag tgggggcctc ctgaggtcag gtggtcttga |
| 1921 | gggagaccag ctacactgat cctgcccttg tcaggagacc taggccttgg gagctatccc |
| 1981 | tgtctgagcc tcaggcctag ggcagtctgt aagctagctg accttggccc tcccggtagc |
| 2041 | ttgacttctt ccctcccctc cgcaggttgg ggcagaggct cctttacctc tggcagtaaa |
| 2101 | ggagcctggg cttcactgag ccccgggttg gtcccctgcc ctctggactt aacctgctgt |
| 2161 | ctcagtgtcc tctgacccct taggggtcca tgtcagtatt ggagtgtgtg ttgaattgtt |
| 2221 | gctccctccc acacactccc gtagccgccc agtttaggat ttccctacac ctgccctaac |
| 2281 | ccacgctttt gggttgggga tcttgccttt ccttgtcatt cccagcagag actgttcctt |
| 2341 | cctgctgtta gaggagtggc ttgtttattc actccaccct gccccctcct gtaaatggag |
| 2401 | aaacaggcct gaaatcaaac gggtaaagcc ctaggccatc cctgtcttcc tgtcccatgt |
| 2461 | ctgcccagtt gaatcccact ggtggcttcc cgggcactga ggagtaaaag cgcctagggc |
| 2521 | tggagaatag gtctgaaatg ggtttgtgac tccccacccc ctgccctgcc ctcaaagctt |
| 2581 | cagacccctc agggagcagc aggatgtggg atcgaggccc cttgggacag atgctttgaa |
| 2641 | tcttccaggg aagcctccga ttcttccagg tttgtcaccc ggagttagca tgtcccaggc |
| 2701 | tcgcagacaa cactgcaggg tgggagacag ctgggcacag ggggattctg ttgagcatgg |
| 2761 | gctctgaacc cacagaactg acaaagcccc tgcttcccca cccccacctc aggctcctgc |
| 2821 | gagcagtgct cctgcaccct tcccagcctg ttctgtactg gggacagcag tcttctccct |
| 2881 | gtcctcccat gtcctatatc cacccctccc cttggaaggt cctccccaca gtgacactgg |
| 2941 | acagccctgg ggcagctgag ccccagcctg gcttctggct ggaagcgcga tgaggagact |
| 3001 | tagcactcca cagtgtccct ggtggtaact gttcttatta actgattgtg ttttgttttg |
| 3061 | ttttgttttg ttttcatgga ccaaaatttt ttttgtactg tctccttaac tgatgtcacc |
| 3121 | cagttttaat aaaagacttc taaagagcag gtc |

SEQ ID NO: 83 Mouse SMARCD1 Amino Acid Sequence (NP_114030.2)

| | |
|---|---|
| 1 | maaragfqsv apsggagasg gagvaaalgp ggtpgppvrm gpapgqglyr spmpgaaypr |
| 61 | pgmlpgsrmt pqgpsmgppg yggnpsvrpg laqsgmdqsr krpapqqiqq vqqqavqnrn |
| 121 | hnakkkkmad kilpqrirel vpesqaymdl laferkldqt imrkrldiqe alkrpikqkr |
| 181 | klrifisntf npaksdaedg egtvaswelr vegrlledaa lskydatkqk rkfssffksl |
| 241 | vieldkdlyg pdnhlvewhr tattqetdgf qvkrpgdvnv rctvllmldy qppqfkldpr |
| 301 | larllgihtq trpviiqalw qyikthklqd pherefvlcd kylqqifesq rmkfseipqr |
| 361 | lhallmppep iiinhvisvd pndqkktacy didvevddtl ktqmnsflls tasqqeiatl |
| 421 | dnkihetiet inqlktqref mlsfardpqg findwlqsqc rdlktmtdvv gnpeeerrae |
| 481 | fyfqpwaqea vcryfyskvq qrrqeleqal girnt |

SEQ ID NO: 84 Human SMARCD2 cDNA Sequence Variant 1 (NM_001098426.1
CDS: 318-1913)

| | |
|---|---|
| 1 | gttgggcggg gcagggagtt cgtagccgcc tctgggtaac tcgactcggg cggccaaacc |
| 61 | tccggaggcc ggggacggaa ggcgggcccg cagcagatcc tggatccgga atctcccggg |
| 121 | caggagcgga atctgtcccg aaccgggtct gtgaggaact cgcgaacttg gattaggaaa |
| 181 | tcccggagcc cggatcgaca aatcccggaa cccggaatta agatcgccaa gtcccggatc |
| 241 | gcggagcaca gagcacggag tggactcgac gcggagcccg gagtccggat cgcggcaccg |
| 301 | cgggacggga cggagcgatg tcgggccgag gcgcgggcgg gttcccgctg cccccgctaa |
| 361 | gccctggcgg cggcgccgtg gctgcggccc tgggagcgcc gcctcccccc gcgggacccg |
| 421 | gcatgctgcc cggaccggcg ctccggggac cgggtccggc aggaggcgtg gggggccccg |
| 481 | gggccgccgc cttccgcccc atgggccccg cgggccccgc ggcgcagtac cagcgacctg |
| 541 | gcatgtcacc agggaaccgg atgcccatgg ctggcttgca ggtgggaccc cctgctggct |
| 601 | ccccatttgg tgcagcagct ccgcttcgac ctggcatgcc acccaccatg atggatccat |
| 661 | tccgaaaacg cctgcttgtg ccccaggcgc agcctcccat gcctgcccag cgccgggggt |
| 721 | taaagaggag gaagatggca gataaggttc tacctcagcg aatccgggag cttgttccag |
| 781 | agtctcaggc gtacatggat ctcttggctt ttgagcggaa gctggaccag accattgctc |
| 841 | gcaagcggat ggagatccag gaggccatca aaaagcctct gacacaaaag cgaaagcttc |
| 901 | ggatctacat ttccaatacg ttcagtccca gcaaggcgga aggcgatagt gcaggaactg |
| 961 | cagggacccc tgggggaacc ccagcagggg acaaggtggc ttcctgggaa ctccgagtgg |
| 1021 | aaggaaaact gctggatgat cctagcaaac agaagaggaa gttttcttca ttctttaaga |
| 1081 | gcctcgtcat tgagctggac aaggagctgt acgggcctga caatcacctg gtggagtggc |
| 1141 | accggatgcc caccacccag gagacagatg gcttccaagt aaaacggcct ggagacctca |
| 1201 | acgtcaagtg caccctcctg ctcatgctgg atcatcagcc tccccagtac aaattggacc |
| 1261 | cccgattggc aaggctgctg ggagtgcaca cgcagacgag ggccgccatc atgcaggccc |
| 1321 | tgtggcttta catcaagcac aaccagctgc aggatgggca cgagcgggag tacatcaact |
| 1381 | gcaaccgtta cttccgccag atcttcagtt gtggccgact ccgtttctcc gagattccca |
| 1441 | tgaagctggc agggttgctg cagcatccag accccattgt catcaaccat gtcattagtg |
| 1501 | tcgaccctaa cgaccagaag aagacagcct gttacgacat cgatgtggag gtggacgacc |
| 1561 | cactgaaggc ccaaatgagc aattttctgg cctctaccac caatcagcag gagatcgcct |
| 1621 | cccttgatgt caagatccat gagaccattg agtccatcaa ccagctgaag acccagagag |
| 1681 | atttcatgct cagttttagc accgaccccc aggacttcat ccaggaatgg ctccgttccc |
| 1741 | agcgccgaga cctcaagatc atcactgatg tgattggaaa tcctgaggag gagagacgag |
| 1801 | ctgctttcta ccaccagccc tgggcccagg aagcagtagg caggcacatc tttgccaagg |
| 1861 | tgcagcagcg aaggcaggaa ctggaacagg tgctgggaat tcgcctgacc taactgctca |
| 1921 | gggatctttc ttcccagccc tggagcctgg agggagacca ccctctgggt ccttgctggg |
| 1981 | gccgcagaca cgtaggctgg ggtgaggagt gtctgctgtc accctctact ctccagcttt |
| 2041 | agtcttataa atgtagtgat aggattcctt gttgcttggt ccccaaagcc ttatactttt |
| 2101 | tgcattggct ttaattgggt tcagcagatg cctcctctgc ccccctgcag gcaggcccaa |
| 2161 | gtaggactgc tggaggctgt gctttgacat tgtaagacat ttccgaacca aaggctgctg |
| 2221 | ggtttgcatg tttacagact ccccctgggg cgagggtcag agctggctct ggggagctgg |
| 2281 | gctaggaaga ggaggtgcag cccagactct tcctagcctt tctaaaccaa agttctttgc |
| 2341 | cattcctaca agcccagcct tgctgctggt tttttccttt cctttgggta tttgcactat |
| 2401 | tttgggagca agttttctat gtgggagcca ctttttttgt acaggggtaa gttgggggtt |
| 2461 | ttcagggagc ctgttaggtg cctccttctt ttctttcctc aatctatgca agcggctctg |
| 2521 | gccgccatca tctcctggga tgccagaggg ctgcctctcc agcggcttgg gccggggagg |
| 2581 | ggacactcca gttctctagc atggcctgag gtatggggta tgtgcatgtg gaggccaggg |
| 2641 | taaggtgaat ggggaggctg ggaggactgg tgttgccctt tggagcttgg tgaggagggt |
| 2701 | gggcctaggg cttggcgagt gccacatctg gcaggtttgg aaatttccaa ataaatcctt |
| 2761 | ttgtctattg |

SEQ ID NO: 85 Human SMARCD2 Amino Acid Sequence Isoform 1
(NP_001091896.1)

| | |
|---|---|
| 1 | msgrgaggfp lpplspggga vaaalgappp pagpgmlpgp alrgpgpagg vggpgaaafr |
| 61 | pmgpagpaaq yqrpgmspgn rmpmaglqvg ppagspfgaa aplrpgmppt mmdpfrkrll |
| 121 | vpqaqppmpa qrrglkrrkm adkvlpqrir elvpesqaym dllaferkld qtiarkrmei |
| 181 | qeaikkpltq krklriyisn tfspskaegd sagtagtpgg tpagdkvasw elrvegklld |
| 241 | dpskqkrkfs sffkslviel dkelygpdnh lvewhrmptt qetdgfqvkr pgdlnvkctl |
| 301 | llmldhqppq ykldprlarl lgvhtqtraa imqalwlyik hnqlqdgher eyincnryfr |
| 361 | qifscgrlrf seipmklagl lqhpdpivin hvisvdpndq kktacydidv evddplkaqm |
| 421 | snflasttnq qeiasldvki hetiesinql ktqrdfmlsf stdpqdfiqe wlrsqrrdlk |
| 481 | iitdvignpe eerraafyhq pwaqeavgrh ifakvqqrrq eleqvlgirl t |

SEQ ID NO:86 Human SMARCD2 cDNA Sequence Variant 2 (NM_001330439.1
CDS: 96-1466)

| | |
|---|---|
| 1 | agtaccaggt gagcaaggag gacgcgagcg gacgggggcg agaggcgctg cgagggcgcc |
| 61 | cgggccggcg gctgaagggg cctcgacgac ctggcatgtc accagggaac cggatgccca |
| 121 | tggctggctt gcaggtggga ccccctgctg gctccccatt tggtgcagca gctccgcttc |
| 181 | gacctggcat gccacccacc atgatggatc cattccgaaa acgcctgctt gtgccccagg |
| 241 | cgcagcctcc catgcctgcc cagcgccggg ggttaaagag gaggaagatg gcagataagg |
| 301 | ttctacctca gcgaatccgg gagcttgttc cagagtctca ggcgtacatg gatctcttgg |
| 361 | cttttgagcg gaagctggac cagaccattg ctcgcaagcg gatggagatc caggaggcca |
| 421 | tcaaaaagcc tctgacacaa aagcgaaagc ttcggatcta catttccaat acgttcagtc |
| 481 | ccagcaaggc ggaaggcgat agtgcaggaa ctgcagggac ccctggggga accccagcag |
| 541 | gggacaaggt ggcttcctgg gaactccgag tggaaggaaa actgctggat gatcctagca |
| 601 | aacagaagag gaagttttct tcattcttta agagcctcgt cattgagctg gacaaggagc |
| 661 | tgtacgggcc tgacaatcac ctggtggagt ggcaccggat gcccaccacc caggagacag |
| 721 | atggcttcca agtaaaacgg cctggagacc tcaacgtcaa gtgcaccctc ctgctcatgc |
| 781 | tggatcatca gcctccccag tacaaattgg acccccgatt ggcaaggctg ctgggagtgc |
| 841 | acacgcagac gagggccgcc atcatgcagg ccctgtggct ttacatcaag cacaaccagc |
| 901 | tgcaggatgg gcacgagcgg gagtacatca actgcaaccg ttacttccgc cagatcttca |
| 961 | gttgtggccg actccgtttc tccgagattc ccatgaagct ggcagggttg ctgcagcatc |
| 1021 | cagaccccat tgtcatcaac catgtcatta gtgtcgaccc taacgaccag aagaagacag |
| 1081 | cctgttacga catcgatgtg gaggtggacg acccactgaa ggcccaaatg agcaattttc |
| 1141 | tggcctctac caccaatcag caggagatcg cctcccttga tgtcaagatc catgagacca |
| 1201 | ttgagtccat caaccagctg aagacccaga gagatttcat gctcagtttt agcaccgacc |
| 1261 | cccaggactt catccaggaa tggctccgtt cccagcgccg agacctcaag atcatcactg |
| 1321 | atgtgattgg aaatcctgag gaggagagac gagctgcttt ctaccaccag ccctgggccc |
| 1381 | aggaagcagt aggcaggcac atctttgcca aggtgcagca gcgaaggcag gaactggaac |
| 1441 | aggtgctggg aattcgcctg acctaactgc tcagggatct ttcttcccag ccctggagcc |
| 1501 | tggagggaga ccaccctctg ggtccttgct ggggccgcag acacgtaggc tggggtgagg |
| 1561 | agtgtctgct gtcaccctct actctccagc tttagtctta taaatgtagt gataggattc |
| 1621 | cttgttgctt ggtccccaaa gccttatact ttttgcattg gctttaattg ggttcagcag |
| 1681 | atgcctcctc tgcccccctg caggcaggcc caagtaggac tgctggaggc tgtgctttga |
| 1741 | cattgtaaga catttccgaa ccaaaggctg ctgggtttgc atgtttacag actccccctg |
| 1801 | gggcgagggt cagagctggc tctggggagc tgggctagga agaggaggtg cagcccagac |
| 1861 | tcttcctagc ctttctaaac caaagttctt tgccattcct acaagcccag ccttgctgct |
| 1921 | ggttttttcc tttcctttgg gtatttgcac tattttggga gcaagttttc tatgtgggag |
| 1981 | ccactttttt tgtacagggg taagttgggg gttttcaggg agcctgttag gtgcctcctt |
| 2041 | cttttctttc ctcaatctat gcaagcggct ctggccgcca tcatctcctg ggatgccaga |
| 2101 | gggctgcctc tccagcggct tgggccgggg aggggacact ccagttctct agcatggcct |
| 2161 | gaggtatggg gtatgtgcat gtggaggcca gggtaaggtg aatggggagg ctgggaggac |
| 2221 | tggtgttgcc ctttggagct tggtgaggag ggtgggccta gggcttggcg agtgccacat |
| 2281 | ctggcaggtt tggaaatttc caaataaatc cttttgtcta ttgaaaaaaa aaaaaaaaaa |
| 2341 | a |

SEQ ID NO: 87 Human SMARCD2 Amino Acid Sequence Isoform 2
(NP_001317368.1)

| | |
|---|---|
| 1 | mspgnrmpma glqvgppags pfgaaaplrp gmpptmmdpf rkrllvpqaq ppmpaqrrgl |
| 61 | krrkmadkvl pqrirelvpe sqaymdllaf erkldqtiar krmeiqeaik kpltqkrklr |
| 121 | iyisntfsps kaegdsagta gtpggtpagd kvaswelrve gkllddpskq krkfssffks |
| 181 | lvieldkely gpdnhlvewh rmpttqetdg fqvkrpgdln vkctlllmld hqppqykldp |
| 241 | rlarllgvht qtraaimqal wlyikhnqlq dghereyinc nryfrqifsc grlrfseipm |
| 301 | klagllqhpd pivinhvisv dpndqkktac ydidvevddp lkaqmsnfla sttnqqeias |
| 361 | ldvkihetie singlktqrd fmlsfstdpq dfiqewlrsq rrdlkiitdv ignpeeerra |
| 421 | afyhqpwage avgrhifakv qqrrqeleqv lgirlt |

SEQ ID NO: 88 Human SMARCD2 cDNA Sequence Variant 3 (NM_001330440.1,
CDS: 48-1499)

| | |
|---|---|
| 1 | agtgtgtgca aggcagagct gccaaacagg ccttgcaggc agcagccatg gggaggcggg |
| 61 | tgggggtgga ggtgactccc agatgggctc cacagaaatg tcagggagca aggcctcagc |
| 121 | gacctggcat gtcaccaggg aaccggatgc ccatggctgg cttgcaggtg ggaccccctg |
| 181 | ctggctcccc atttggtgca gcagctccgc ttcgacctgg catgccaccc accatgatgg |
| 241 | atccattccg aaaacgcctg cttgtgcccc aggcgcagcc tcccatgcct gcccagcgcc |
| 301 | gggggttaaa gaggaggaag atggcagata aggttctacc tcagcgaatc cgggagcttg |
| 361 | ttccagagtc tcaggcgtac atggatctct tggcttttga gcggaagctg gaccagacca |
| 421 | ttgctcgcaa gcggatggag atccaggagg ccatcaaaaa gcctctgaca caaaagcgaa |
| 481 | agcttcggat ctacatttcc aatacgttca gtcccagcaa ggcggaaggc gatagtgcag |
| 541 | gaactgcagg gacccctggg ggaaccccag caggggacaa ggtggcttcc tgggaactcc |
| 601 | gagtggaagg aaaactgctg gatgatccta gcaaacagaa gaggaagttt tcttcattct |
| 661 | ttaagagcct cgtcattgag ctggacaagg agctgtacgg gcctgacaat cacctggtgg |
| 721 | agtggcaccg gatgcccacc acccaggaga cagatggctt ccaagtaaaa cggcctggag |
| 781 | acctcaacgt caagtgcacc ctcctgctca tgctggatca tcagcctccc cagtacaaat |
| 841 | tggacccccg attggcaagg ctgctgggag tgcacacgca gacgagggcc gccatcatgc |
| 901 | aggccctgtg gctttacatc aagcacaacc agctgcagga tgggcacgag cgggagtaca |
| 961 | tcaactgcaa ccgttacttc cgccagatct tcagttgtgg ccgactccgt ttctccgaga |
| 1021 | ttcccatgaa gctggcaggg ttgctgcagc atccagaccc cattgtcatc aaccatgtca |
| 1081 | ttagtgtcga ccctaacgac cagaagaaga cagcctgtta cgacatcgat gtggaggtgg |
| 1141 | acgacccact gaaggcccaa atgagcaatt ttctggcctc taccaccaat cagcaggaga |
| 1201 | tcgcctccct tgatgtcaag atccatgaga ccattgagtc catcaaccag ctgaagaccc |
| 1261 | agagagattt catgctcagt tttagcaccg acccccagga cttcatccag gaatggctcc |
| 1321 | gttcccagcg ccgagacctc aagatcatca ctgatgtgat tggaaatcct gaggaggaga |
| 1381 | gacgagctgc tttctaccac cagccctggg cccaggaagc agtaggcagg cacatctttg |
| 1441 | ccaaggtgca gcagcgaagg caggaactgg aacaggtgct gggaattcgc ctgacctaac |
| 1501 | tgctcaggga tctttcttcc cagccctgga gcctggaggg agaccaccct ctgggtcctt |
| 1561 | gctggggccg cagacacgta ggctggggtg aggagtgtct gctgtcaccc tctactctcc |
| 1621 | agctttagtc ttataaatgt agtgatagga ttccttgttg cttggtcccc aaagccttat |
| 1681 | actttttgca ttggctttaa ttgggttcag cagatgcctc ctctgccccc ctgcaggcag |
| 1741 | gcccaagtag gactgctgga ggctgtgctt tgacattgta agacatttcc gaaccaaagg |
| 1801 | ctgctgggtt tgcatgttta cagactcccc ctggggcgag ggtcagagct ggctctgggg |
| 1861 | agctgggcta ggaagaggag gtgcagccca gactcttcct agcctttcta aaccaaagtt |
| 1921 | ctttgccatt cctacaagcc cagccttgct gctggttttt tcctttcctt tgggtatttg |
| 1981 | cactattttg ggagcaagtt ttctatgtgg gagccacttt ttttgtacag gggtaagttg |
| 2041 | ggggttttca gggagcctgt taggtgcctc cttcttttct ttcctcaatc tatgcaagcg |
| 2101 | gctctggccg ccatcatctc ctgggatgcc agagggctgc ctctccagcg gcttgggccg |
| 2161 | gggaggggac actccagttc tctagcatgg cctgaggtat ggggtatgtg catgtggagg |
| 2221 | ccagggtaag gtgaatgggg aggctgggag gactggtgtt gccctttgga gcttggtgag |
| 2281 | gagggtgggc ctagggcttg gcgagtgcca catctggcag gtttggaaat ttccaaataa |
| 2341 | atccttttgt ctattgaaaa aaaaaaaaaa aaaa |

SEQ ID NO: 89 Human SMARCD2 Amino Acid Sequence Isoform 3
(NP_001317369.1)

| | |
|---|---|
| 1 | mgrrvgvevt prwapqkcqg arpqrpgmsp gnrmpmaglq vgppagspfg aaaplrpgmp |
| 61 | ptmmdpfrkr llvpqaqppm paqrrglkrr kmadkvlpqr irelvpesqa ymdllaferk |
| 121 | ldqtiarkrm eiqeaikkpl tqkrklriyi sntfspskae gdsagtagtp ggtpagdkva |
| 181 | swelrvegkl lddpskqkrk fssffkslvi eldkelygpd nhlvewhrmp ttqetdgfqv |
| 241 | krpgdlnvkc tlllmldhqp pqykldprla rllgvhtqtr aaimqalwly ikhnqlqdgh |
| 301 | ereyincnry frqifscgrl rfseipmkla gllqhpdpiv inhvisvdpn dqkktacydi |
| 361 | dvevddplka qmsnflastt nqqeiasldv kihetiesin qlktqrdfml sfstdpqdfi |
| 421 | qewlrsqrrd lkiitdvign peeerraafy hqpwageavg rhifakvqqr rqeleqvlgi |
| 481 | rlt |

SEQ ID NO: 90 Mouse SMARCD2 cDNA Sequence Variant 1 (NM_001130187.1,
CDS: 265-1860)

| | |
|---|---|
| 1 | ctccggcgat caaacctccg gaggccggga gaggcctgcg ggctcgcggc acatcccgga |
| 61 | tctggagtat ccctggcagg agcggagtca gaggggccgc gggatcctaa agccgggctg |
| 121 | caaagaactt gcgaacttgg agtagaagat cccggaaccc ggtagtaaaa tcgggaagtc |
| 181 | ccggatcgcg gaacgtagct cgcggagcgg actcaacacg gagaccggag gccggatcgc |
| 241 | tgcaccgcgg gacgggacag agtgatgtcc ggccgtggcg cgggcgggtt cccgctgcct |
| 301 | ccgctgagcc ccggcggcgg cgccgttgcc gcggcccttg gtgcgccgcc tccgcctgcg |
| 361 | ggacccggaa tgctgcccag cccggcgctc aggggcccgg ggccttctgg aggcatgggg |
| 421 | gtaccggggg ccgccgcctt ccgccccatg ggccccgctg gccccgcggc gcagtaccag |
| 481 | cgtcctggca tgtcaccagg aagcaggatg cccatggctg gcttgcaggt gggacctcct |
| 541 | gccggttccc catttggcac agctgctccg ctccgacctg gcatgccacc taccatgatg |
| 601 | gatccattcc gaaaacgcct gcttgtgcct caggcccagc ccccgatgcc tgcccagcgc |
| 661 | cgagggttaa agaggaggaa gatggcagat aaggttctac ctcagcgaat ccgggagctt |
| 721 | gtcccagagt ctcaggcata catggatctt ttagctttcg agaggaagct ggaccagacc |
| 781 | atcgctcgca agcggatgga gattcaagag gccatcaaga agcctctgac gcaaaagcga |
| 841 | aaacttcgga tctatatttc caatacattc agccccagca aggcggatgg agataatgcg |
| 901 | ggaactgcgg ggacccctgg gggaaccccg gcagcagaca aggtggcctc ctgggagctt |
| 961 | cgagtagagg ggaaactgct ggatgatcct agcaaacaga agaggaagtt ctcatcattc |
| 1021 | tttaagagcc ttgtgattga gttggacaag gaactctatg ggccggacaa ccatctggtg |
| 1081 | gagtggcatc ggatgcccac cacacaggaa acagatggct ttcaggtgaa acggccagga |
| 1141 | gatctcaatg tcaagtgcac ccttctgctc atgctggatc atcagcctcc tcagtataaa |
| 1201 | ctggaccccc gcctggcgag gttgctggga gtgcacacac agaccagggc ggcaatcatg |
| 1261 | caggcactgt ggctttacat caaacacaac cagctgcagg acggccatga gcgcgagtac |
| 1321 | atcaactgca atcgttactt ccgccagatc ttcagttgtg gccgactccg tttctccgag |
| 1381 | attcccatga agctggctgg attgctgcag catccagacc ccattgttat taatcatgtc |
| 1441 | attagtgtgg atcctaatga ccaaaagaag acagcctgct atgacattga tgtagaggtt |
| 1501 | gatgacccac tgaaggccca gatgagcaac ttcctggcct ctaccaccaa ccagcaggag |
| 1561 | attgcttctc ttgacgtcaa gatccatgag accattgagt ccatcaacca gctaaagacc |
| 1621 | cagagggatt tcatgctcag ctttagcacc gagccccagg acttcatcca ggagtggctc |
| 1681 | cgttcccaac gccgagacct caagatcatc acagatgtga ttggaaaccc tgaggaggag |
| 1741 | agacgagctg ctttctacca ccagccctgg gctcaggaag cagtggggag gcacatcttt |
| 1801 | gccaaggtgc agcagcgaag gcaggaactg gaacaggtgc tgggaattcg cctgacctaa |
| 1861 | ctgctcaggg attgcctcct tccttcctcc cctgccctgg atggaacctg gcaagagccc |
| 1921 | gtcctctggg ttctggcttg ggctgcagac atgtaggatg gagtgaggtg tgtttcctgt |
| 1981 | caccctccac tccccagctt tagtttcata aatgtagttt tagatccctc actgcttggt |
| 2041 | tcccaaagcc ttattactga ccttttagcg ctggctttaa ttgggtttgc aatgagcggc |
| 2101 | ctcagccccc tgcaggcagg caggcctgag taggaggctg gaggctgtgc tttaactttg |
| 2161 | taccagacat ttccaaacca aaggctgctg ggtttgcatg tttacaggct ccaccctagg |
| 2221 | gccagtgcca gagctggctt tggggagctg ggcaaggaag agaaggccct agactcttcc |
| 2281 | tggcctttct aaccaaagtt ttttgccatt cctacaagcc cagtcttgct gctggtttgt |
| 2341 | ccttcttttt gggtatttgc actatttggg gagcaggttt ttctatgtgg gagccacttt |
| 2401 | tttgtacaga ggtaatgggg tttttcaggg agcccacttg gtgcctcctt cttcctttct |
| 2461 | tttcttaatc tatgcaagcg gctgcagccg ccatcatctc ctggtatgcc acaaggctgc |
| 2521 | ccacccatag ctgcttgggc agggggaggt ggaatctcct gagagtggca atgccagttc |
| 2581 | tctaacccag ttacagcagg ggtgtgtgtg cgtgcgtgcg tgcgtgctgc aggggaaggg |
| 2641 | gaaagctgga ggactgctgt taccttttgc agtcggtctt aaagaggatg ggcctaaggc |
| 2701 | ttggcaaact tggaaaattc caaataaatc tttttgttta ttggtggtgc ccagaaaaaa |
| 2761 | aaaaaaa |

SEQ ID NO: 91 Mouse SMARCD2 Amino Acid Sequence Isoform 1
(NP_001123659.1)

| | |
|---|---|
| 1 | msgrgaggfp lpplspggga vaaalgappp pagpgmlpsp alrgpgpsgg mgvpgaaafr |
| 61 | pmgpagpaaq yqrpgmspgs rmpmaglqvg ppagspfgta aplrpgmppt mmdpfrkrll |
| 121 | vpqaqppmpa qrrglkrrkm adkvlpqrir elvpesqaym dllaferkld qtiarkrmei |
| 181 | qeaikkpltq krklriyisn tfspskadgd nagtagtpgg tpaadkvasw elrvegklld |
| 241 | dpskqkrkfs sffkslviel dkelygpdnh lvewhrmptt qetdgfqvkr pgdlnvkctl |
| 301 | LLmldhqppq ykldprlarl lgvhtqtraa imqalwlyik hnqlqdgher eyincnryfr |
| 361 | qifscgrlrf seipmklagl lqhpdpivin hvisvdpndq kktacydidv evddplkaqm |
| 421 | snflasttnq qeiasldvki hetiesinql ktqrdfmlsf stepqdfiqe wlrsqrrdlk |
| 481 | iitdvignpe eerraafyhq pwaqeavgrh ifakvqqrrq eleqvlgirl t |

SEQ ID NO: 92 Mouse SMARCD2 cDNA Sequence Variant 2 (NM 031878.2, CDS:
40-1494)

| | |
|---|---|
| 1 | tttgttcctg gtctccccat ttgagagaga gagagagaga tggagggtat gggctatgga |
| 61 | cctcggaggg ctccgccact gacctgtgtc cctccactgt tccactttcc tcagcgtcct |
| 121 | ggcatgtcac caggaagcag gatgcccatg gctggcttgc aggtgggacc tcctgccggt |
| 181 | tccccatttg gcacagctgc tccgctccga cctggcatgc cacctaccat gatggatcca |
| 241 | ttccgaaaac gcctgcttgt gcctcaggcc cagcccccga tgcctgccca gcgccgaggg |
| 301 | ttaaagagga ggaagatggc agataaggtt ctacctcagc gaatccggga gcttgtccca |
| 361 | gagtctcagg catacatgga tcttttagct ttcgagagga agctggacca gaccatcgct |
| 421 | cgcaagcgga tggagattca agaggccatc aagaagcctc tgacgcaaaa gcgaaaactt |
| 481 | cggatctata tttccaatac attcagcccc agcaaggcgg atggagataa tgcgggaact |
| 541 | gcggggaccc ctgggggaac cccggcagca gacaaggtgg cctcctggga gcttcgagta |
| 601 | gaggggaaac tgctggatga tcctagcaaa cagaagagga agttctcatc attctttaag |
| 661 | agccttgtga ttgagttgga caaggaactc tatgggccgg acaaccatct ggtggagtgg |
| 721 | catcggatgc ccaccacaca ggaaacagat ggctttcagg tgaaacggcc aggagatctc |
| 781 | aatgtcaagt gcacccttct gctcatgctg gatcatcagc ctcctcagta taaactggac |
| 841 | ccccgcctgg cgaggttgct gggagtgcac acacagacca gggcggcaat catgcaggca |
| 901 | ctgtggcttt acatcaaaca caaccagctg caggacggcc atgagcgcga gtacatcaac |
| 961 | tgcaatcgtt acttccgcca gatcttcagt tgtggccgac tccgtttctc cgagattccc |
| 1021 | atgaagctgg ctggattgct gcagcatcca gaccccattg ttattaatca tgtcattagt |
| 1081 | gtggatccta atgaccaaaa gaagacagcc tgctatgaca ttgatgtaga ggttgatgac |
| 1141 | ccactgaagg cccagatgag caacttcctg gcctctacca ccaaccagca ggagattgct |
| 1201 | tctcttgacg tcaagatcca tgagaccatt gagtccatca accagctaaa gacccagagg |
| 1261 | gatttcatgc tcagctttag caccgagccc caggacttca tccaggagtg gctccgttcc |
| 1321 | caacgccgag acctcaagat catcacagat gtgattggaa accctgagga ggagagacga |
| 1381 | gctgctttct accaccagcc ctgggctcag gaagcagtgg ggaggcacat ctttgccaag |
| 1441 | gtgcagcagc gaaggcagga actggaacag gtgctgggaa ttcgcctgac ctaactgctc |
| 1501 | agggattgcc tccttccttc ctcccctgcc ctggatggaa cctggcaaga gcccgtcctc |
| 1561 | tgggttctgg cttgggctgc agacatgtag gatggagtga ggtgtgtttc ctgtcaccct |
| 1621 | ccactcccca gctttagttt cataaatgta gttttagatc cctcactgct tggttcccaa |
| 1681 | agccttatta ctgacctttt agcgctggct ttaattgggt ttgcaatgag cggcctcagc |
| 1741 | cccctgcagg caggcaggcc tgagtaggag gctggaggct gtgctttaac tttgtaccag |
| 1801 | acatttccaa accaaaggct gctgggtttg catgtttaca ggctccaccc tagggccagt |
| 1861 | gccagagctg gctttgggga gctgggcaag gaagagaagg ccctagactc ttcctggcct |
| 1921 | ttctaaccaa agttttttgc cattcctaca agcccagtct tgctgctggt ttgtccttct |
| 1981 | ttttgggtat ttgcactatt tggggagcag gtttttctat gtgggagcca cttttttgta |
| 2041 | cagaggtaat ggggtttttc agggagccca cttggtgcct ccttcttcct ttcttttctt |
| 2101 | aatctatgca agcggctgca gccgccatca tctcctggta tgccacaagg ctgcccaccc |
| 2161 | atagctgctt gggcaggggg aggtggaatc tcctgagagt ggcaatgcca gttctctaac |
| 2221 | ccagttacag caggggtgtg tgtgcgtgcg tgcgtgcgtg ctgcagggga aggggaaagc |
| 2281 | tggaggactg ctgttacctt ttgcagtcgg tcttaaagag gatgggccta aggcttggca |
| 2341 | aacttggaaa attccaaata aatctttttg tttattggtg gtgcccagaa aaaaaaaaaa |
| 2401 | a |

SEQ ID NO: 93 Mouse SMARCD2 Amino Acid Sequence Isoform 2 (NP 114084.2)

| | |
|---|---|
| 1 | megmgygprr appltcvppl fhfpqrpgms pgsrmpmagl qvgppagspf gtaaplrpgm |
| 61 | pptmmdpfrk rllvpqaqpp mpaqrrglkr rkmadkvlpq rirelvpesq aymdllafer |
| 121 | kldqtiarkr meiqeaikkp ltqkrklriy isntfspska dgdnagtagt pggtpaadkv |
| 181 | aswelrvegk llddpskqkr kfssffkslv ieldkelygp dnhlvewhrm pttqetdgfq |
| 241 | vkrpgdlnvk ctlllmldhq ppqykldprl arllgvhtqt raaimqalwl yikhnqlqdg |
| 301 | hereyincnr yfrqifscgr lrfseipmkl agllqhpdpi vinhvisvdp ndqkktacyd |
| 361 | idvevddplk aqmsnflast tnqqeiasld vkihetiesi nqlktqrdfm lsfstepqdf |
| 421 | iqewlrsqrr dlkiitdvig npeeerraaf yhqpwageav grhifakvqq rrqeleqvlg |
| 481 | irlt |

SEQ ID NO: 94 Human SMARCD3 cDNA Sequence Variant 1 (NM 001003802.1,
CDS: 130-1542)

| | |
|---|---|
| 1 | ctggcatctt cctcccctcc tcctttccag atcctcagaa tggcccttgg tgctgcaggc |
| 61 | gcggtgggct ccgggcccag gcaccgaggg ggcactggat gactctccag gtgcaggacc |
| 121 | ctgccatcta tgactccagg tcttcagcac ccacccaccg tggtacagcg ccccgggatg |
| 181 | ccgtctggag cccggatgcc ccaccagggg gcgcccatgg gccccccggg ctccccgtac |
| 241 | atgggcagcc ccgccgtgcg acccggcctg gcccccgcgg gcatggagcc cgcccgcaag |
| 301 | cgagcagcgc ccccgcccgg gcagagccag gcacagagcc agggccagcc ggtgcccacc |
| 361 | gcccccgcgc ggagccgcag tgccaagagg aggaagatgg ctgacaaaat cctccctcaa |
| 421 | aggattcggg agctggtccc cgagtcccag gcttacatgg acctcttggc atttgagagg |
| 481 | aaactggatc aaaccatcat gcggaagcgg gtggacatcc aggaggctct gaagaggccc |
| 541 | atgaagcaaa agcggaagct gcgactctat atctccaaca cttttaaccc tgcgaagcct |
| 601 | gatgctgagg attccgacgg cagcattgcc tcctgggagc tacgggtgga ggggaagctc |
| 661 | ctggatgatc ccagcaaaca gaagcggaag ttctcttctt tcttcaagag tttggtcatc |
| 721 | gagctggaca aagatcttta tggccctgac aaccacctcg ttgagtggca tcggacaccc |
| 781 | acgacccagg agacggacgg cttccaggtg aaacggcctg gggacctgag tgtgcgctgc |
| 841 | acgctgctcc tcatgctgga ctaccagcct ccccagttca aactggatcc ccgcctagcc |
| 901 | cggctgctgg ggctgcacac acagagccgc tcagccattg tccaggccct gtggcagtat |
| 961 | gtgaagacca acaggctgca ggactcccat gacaaggaat acatcaatgg ggacaagtat |
| 1021 | ttccagcaga tttttgattg tccccggctg aagttttctg agattcccca gcgcctcaca |
| 1081 | gccctgctat tgccccctga cccaattgtc atcaaccatg tcatcagcgt ggacccttca |
| 1141 | gaccagaaga agacggcgtg ctatgacatt gacgtggagg tggaggagcc attaaagggg |
| 1201 | cagatgagca gcttcctcct atccacggcc aaccagcagg agatcagtgc tctggacagt |
| 1261 | aagatccatg agacgattga gtccataaac cagctcaaga tccagaggga cttcatgcta |
| 1321 | agcttctcca gagaccccaa aggctatgtc caagacctgc tccgctccca gagccgggac |
| 1381 | ctcaaggtga tgacagatgt agccggcaac cctgaagagg agcgccgggc tgagttctac |
| 1441 | caccagccct ggtcccagga ggccgtcagt cgctacttct actgcaagat ccagcagcgc |
| 1501 | aggcaggagc tggagcagtc gctggttgtg cgcaacacct aggagcccaa aaataagcag |
| 1561 | cacgacggaa ctttcagccg tgtcccgggc cccagcattt tgccccgggc tccagcatca |
| 1621 | ctcctctgcc accttggggt gtggggctgg attaaaagtc attcatctga caaaaaaaaa |
| 1681 | aaaaaaaaa |

SEQ ID NO: 95 Human SMARCD3 Amino Acid Sequence Isoform 1
(NP_001003802.1 and NP_003069.2)

| | |
|---|---|
| 1 | mtpglqhppt vvqrpgmpsg armphqgapm gppgspymgs pavrpglapa gmeparkraa |
| 61 | pppgqsqaqs qgqpvptapa rsrsakrrkm adkilpqrir elvpesqaym dllaferkld |
| 121 | qtimrkrvdi qealkrpmkq krklrlyisn tfnpakpdae dsdgsiaswe lrvegklldd |
| 181 | pskqkrkfss ffkslvield kdlygpdnhl vewhrtpttq etdgfqvkrp gdlsvrctll |
| 241 | lmldyqppqf kldprlarll glhtqsrsai vqalwqyvkt nrlqdshdke yingdkyfqq |
| 301 | ifdcprlkfs eipqrltall lppdpivinh visvdpsdqk ktacydidve veeplkgqms |
| 361 | sfllstanqq eisaldskih etiesinqlk iqrdfmlsfs rdpkgyvqdl lrsqsrdlkv |
| 421 | mtdvagnpee erraefyhqp wsqeavsryf yckiqqrrqe leqslvvrnt |

SEQ ID NO: 96 Human SMARCD3 cDNA Sequence Variant 2 (NM_003078.3,
CDS: 169-1581)

| | |
|---|---|
| 1 | gccgggccga gccgagcgcc gagcagggag cgggcggccg cgctccgggc cggggtcccg |
| 61 | ggggagcaga tcctcagaat ggcccttggt gctgcaggcg cggtgggctc cgggcccagg |
| 121 | caccgagggg gcactggatg actctccagg tgcaggaccc tgccatctat gactccaggt |
| 181 | cttcagcacc cacccaccgt ggtacagcgc cccgggatgc cgtctggagc ccggatgccc |
| 241 | caccaggggg cgcccatggg ccccccgggc tccccgtaca tgggcagccc cgccgtgcga |
| 301 | cccggcctgg cccccgcggg catggagccc gcccgcaagc gagcagcgcc cccgcccggg |
| 361 | cagagccagg cacagagcca gggccagccg gtgcccaccg cccccgcgcg gagccgcagt |
| 421 | gccaagagga ggaagatggc tgacaaaatc ctccctcaaa ggattcggga gctggtcccc |
| 481 | gagtcccagg cttacatgga cctcttggca tttgagagga aactggatca aaccatcatg |
| 541 | cggaagcggg tggacatcca ggaggctctg aagaggccca tgaagcaaaa gcggaagctg |
| 601 | cgactctata tctccaacac ttttaaccct gcgaagcctg atgctgagga ttccgacggc |
| 661 | agcattgcct cctgggagct acgggtggag gggaagctcc tggatgatcc cagcaaacag |
| 721 | aagcggaagt tctcttcttt cttcaagagt ttggtcatcg agctggacaa agatctttat |
| 781 | ggccctgaca accacctcgt tgagtggcat cggacaccca cgacccagga gacggacggc |
| 841 | ttccaggtga aacggcctgg ggacctgagt gtgcgctgca cgctgctcct catgctggac |
| 901 | taccagcctc cccagttcaa actggatccc cgcctagccc ggctgctggg gctgcacaca |
| 961 | cagagccgct cagccattgt ccaggccctg tggcagtatg tgaagaccaa caggctgcag |
| 1021 | gactcccatg acaaggaata catcaatggg gacaagtatt tccagcagat ttttgattgt |
| 1081 | ccccggctga agttttctga gattccccag cgcctcacag ccctgctatt gccccctgac |
| 1141 | ccaattgtca tcaaccatgt catcagcgtg gacccttcag accagaagaa gacggcgtgc |
| 1201 | tatgacattg acgtggaggt ggaggagcca ttaaaggggc agatgagcag cttcctccta |
| 1261 | tccacggcca accagcagga gatcagtgct ctggacagta agatccatga gacgattgag |
| 1321 | tccataaacc agctcaagat ccagagggac ttcatgctaa gcttctccag agaccccaaa |
| 1381 | ggctatgtcc aagacctgct ccgctcccag agccgggacc tcaaggtgat gacagatgta |
| 1441 | gccggcaacc ctgaagagga gcgccgggct gagttctacc accagccctg gtcccaggag |
| 1501 | gccgtcagtc gctacttcta ctgcaagatc cagcagcgca ggcaggagct ggagcagtcg |
| 1561 | ctggttgtgc gcaacaccta ggagcccaaa aataagcagc acgacggaac tttcagccgt |
| 1621 | gtcccgggcc ccagcatttt gccccgggct ccagcatcac tcctctgcca ccttggggtg |
| 1681 | tggggctgga ttaaaagtca ttcatctgac aaaaaaaaaa aaaaaaaa |

SEQ ID NO: 97 Human SMARCD3 Amino Acid Sequence Isoform 2
(NP_001317368.1)

| | |
|---|---|
| 1 | mspgnrmpma glqvgppags pfgaaaplrp gmpptmmdpf rkrllvpqaq ppmpaqrrgl |
| 61 | krrkmadkvl pqrirelvpe sqaymdllaf erkldqtiar krmeiqeaik kpltqkrklr |
| 121 | iyisntfsps kaegdsagta gtpggtpagd kvaswelrve gkllddpskq krkfssffks |
| 181 | lvieldkely gpdnhlvewh rmpttqetdg fqvkrpgdln vkctlllmld hqppqykldp |
| 241 | rlarllgvht qtraaimqal wlyikhnqlq dghereyinc nryfrqifsc grlrfseipm |
| 301 | klagllqhpd pivinhvisv dpndqkktac ydidvevddp lkaqmsnfla sttnqqeias |
| 361 | ldvkihetie sinqlktqrd fmlsfstdpq dfiqewlrsq rrdlkiitdv ignpeeerra |
| 421 | afyhqpwaqe avgrhifakv qqrrqeleqv lgirlt |

SEQ ID NO: 98 Human SMARCD3 cDNA Sequence Variant 3 (NM_001003801.1,
CDS: 102-1553)

| | |
|---|---|
| 1 | agcaggactc agaggggaga gttggaggaa aaaaaaaggc agaaaaggga aagaaagagg |
| 61 | aagagagaga gagagtgaga ggagccgctg agcccacccc gatggccgcg gacgaagttg |
| 121 | ccggaggggc gcgcaaagcc acgaaaagca aactttttga gtttctggtc catggggtgc |
| 181 | gccccgggat gccgtctgga gcccggatgc cccaccaggg ggcgcccatg ggccccccgg |
| 241 | gctccccgta catgggcagc cccgccgtgc gacccggcct ggcccccgcg ggcatggagc |
| 301 | ccgcccgcaa gcgagcagcg cccccgcccg ggcagagcca ggcacagagc cagggccagc |
| 361 | cggtgcccac cgcccccgcg cggagccgca gtgccaagag gaggaagatg gctgacaaaa |
| 421 | tcctccctca aaggattcgg gagctggtcc ccgagtccca ggcttacatg gacctcttgg |
| 481 | catttgagag gaaactggat caaaccatca tgcggaagcg ggtggacatc caggaggctc |
| 541 | tgaagaggcc catgaagcaa aagcggaagc tgcgactcta tatctccaac acttttaacc |
| 601 | ctgcgaagcc tgatgctgag gattccgacg gcagcattgc ctcctgggag ctacgggtgg |
| 661 | aggggaagct cctggatgat cccagcaaac agaagcggaa gttctcttct ttcttcaaga |
| 721 | gtttggtcat cgagctggac aaagatcttt atggccctga caaccacctc gttgagtggc |
| 781 | atcggacacc cacgacccag gagacggacg gcttccaggt gaaacggcct ggggacctga |
| 841 | gtgtgcgctg cacgctgctc ctcatgctgg actaccagcc tccccagttc aaactggatc |
| 901 | cccgcctagc ccggctgctg gggctgcaca cacagagccg ctcagccatt gtccaggccc |
| 961 | tgtggcagta tgtgaagacc aacaggctgc aggactccca tgacaaggaa tacatcaatg |
| 1021 | gggacaagta tttccagcag atttttgatt gtccccggct gaagttttct gagattcccc |
| 1081 | agcgcctcac agccctgcta ttgccccctg acccaattgt catcaaccat gtcatcagcg |
| 1141 | tggacccttc agaccagaag aagacggcgt gctatgacat tgacgtggag gtggaggagc |
| 1201 | cattaaaggg gcagatgagc agcttcctcc tatccacggc caaccagcag gagatcagtg |
| 1261 | ctctggacag taagatccat gagacgattg agtccataaa ccagctcaag atccagaggg |
| 1321 | acttcatgct aagcttctcc agagacccca aaggctatgt ccaagacctg ctccgctccc |
| 1381 | agagccggga cctcaaggtg atgacagatg tagccggcaa ccctgaagag gagcgccggg |
| 1441 | ctgagttcta ccaccagccc tggtcccagg aggccgtcag tcgctacttc tactgcaaga |
| 1501 | tccagcagcg caggcaggag ctggagcagt cgctggttgt gcgcaacacc taggagccca |
| 1561 | aaaataagca gcacgacgga actttcagcc gtgtcccggg ccccagcatt ttgccccggg |
| 1621 | ctccagcatc actcctctgc caccttgggg tgtggggctg gattaaaagt cattcatctg |
| 1681 | acaaaaaaaa aaaaaaaaaa |

SEQ ID NO: 99 Mouse SMARCD3 cDNA Sequence (NM_025891.3, CDS: 145-
1596)

| | |
|---|---|
| 1 | gggccccctc cccactccgc tcgagtagaa gtgtgagaga gcccagcagg actcagaggg |
| 61 | gagagttgga ggaaaaaaaa ggcagaaaag ggaaagaaag aggaagagag agagagagtg |
| 121 | agaggagccg ctgagcccac cccgatggcc gcggacgaag ttgccggagg ggcgcgcaaa |
| 181 | gccacgaaaa gcaaactttt tgagtttctg gtccatgggg tgcgccccgg gatgccgtct |
| 241 | ggagcccgaa tgccccacca gggggcgccc atgggccccc cgggctcccc gtacatgggc |
| 301 | agccccgcgg tacgacccgg cctggccccc gcgggcatgg agcccgcccg caagcgagca |
| 361 | gcgcccccgc ccgggcagag ccaggcacag ggccagggcc agcccgtgcc caccgcccca |
| 421 | gcgcggagcc gcagtgccaa gaggaggaag atggctgaca aaatcctccc tcaaaggatt |
| 481 | cgggagctgg tacccgagtc ccaggcttac atggacctcc tagcatttga gaggaaactg |
| 541 | gatcaaacca tcatgcggaa gcgggtggac atccaggagg ccctgaagag gcccatgaag |
| 601 | caaaagcgaa agctgcgcct ttatatctcc aatactttta accctgcgaa gcctgatgcg |
| 661 | gaagactctg atggcagcat tgcctcctgg gagctgcggg tggaggggaa gctcttggat |
| 721 | gatcctagta agcagaagag gaagttttct tccttcttca agagtttggt cattgagttg |
| 781 | gacaaagacc tttatggccc agacaaccac cttgttgagt ggcaccggac acccacaacc |
| 841 | caggaaacag atgggttcca agtgaagaga ccaggggact tgagtgtgcg ctgcaccctg |
| 901 | ctcctgatgc tggactatca gcctccccag ttcaaattgg acccccgctt agcccggctg |
| 961 | ctggggttac acacacagag ccgctcagcc attgtccagg cactgtggca gtatgtgaag |
| 1021 | accaacaggc tacaggactc ccatgacaag gagtacatca atggcgacaa gtatttccag |
| 1081 | cagatttttg actgcccccg cctaaagttc tctgagattc cccagcgcct cacagccctg |
| 1141 | ctgctgcccc ctgaccccat tgtgatcaac cacgtcatca gcgtggaccc atcagaccag |
| 1201 | aagaagacag cgtgctatga catagatgtg gaggtggagg aaccgctgaa agggcagatg |
| 1261 | agtagcttcc tcctgtccac ggccaaccag caggagatca gtgctctgga cagtaagatc |
| 1321 | catgagacga ttgagtccat aaaccagctc aagatccaga gggacttcat gctaagtttc |
| 1381 | tccagagacc ccaaaggcta cgtccaagac ctgctccgct cccagagccg tgatctcaag |
| 1441 | gtgatgacag atgtggcagg gaaccccgag gaagaacgca gggctgagtt ctaccaccag |
| 1501 | ccctggtccc aggaagccgt tagccgctac ttctactgta agatccagca gcgcaggcag |
| 1561 | gagctggagc agtcgctggt cgtgcgcaac acctaggagc ccgtgaacaa gcgtcagggt |
| 1621 | ggaccagcca ctccgcccag cacaggccct gggctctgga ctccccctct cgcgctgtgc |
| 1681 | ggaaggtggg gagggctgga tggattaaag gtcacgtaac agacaaaaaa aaaaaaaaaa |
| 1741 | aaa |

SEQ ID NO: 100 Mouse SMARCD3 Amino Acid Sequence (NP_080167.3)

| | |
|---|---|
| 1 | maadevagga rkatksklfe flvhgvrpgm psgarmphqg apmgppgspy mgspavrpgl |
| 61 | apagmepark raapppgqsq aqgqgqpvpt aparsrsakr rkmadkilpq rirelvpesq |
| 121 | aymdllafer kldqtimrkr vdiqealkrp mkqkrklrly isntfnpakp daedsdgsia |
| 181 | swelrvegkl lddpskqkrk fssffkslvi eldkdlygpd nhlvewhrtp ttqetdgfqv |
| 241 | krpgdlsvrc tlllmldyqp pqfkldprla rllglhtqsr saivqalwqy vktnrlqdsh |
| 301 | dkeyingdky fqqifdcprl kfseipqrlt alllppdpiv inhvisvdps dqkktacydi |
| 361 | dveveeplkg qmssfllsta nqqeisalds kihetiesin qlkiqrdfml sfsrdpkgyv |
| 421 | qdllrsqsrd lkvmtdvagn peeerraefy hqpwsqeavs ryfyckiqqr rqeleqslvv |
| 481 | rnt |

SEQ ID NO: 101 human SMARCE1 cDNA Sequence (NM_003079.4, CDS: 125-
1360)

| | |
|---|---|
| 1 | gctccggacg cgaggggcgg ggcgagcgcg ggacaaaggg aagcgaagcc ggagctgcgg |
| 61 | gcgctttttc tgcccgcggt gtctcagatt cattcttaag gaactgagaa cttaatcttc |
| 121 | caaaatgtca aaaagaccat cttatgcccc acctcccacc ccagctcctg caacacaaat |
| 181 | gcccagcaca ccagggtttg tgggatacaa tccatacagt catctcgcct acaacaacta |
| 241 | caggctggga gggaacccgg gcaccaacag ccgggtcacg gcatcctctg gtatcacgat |
| 301 | tccaaaaccc ccaaagccac cagataagcc gctgatgccc tacatgaggt acagcagaaa |
| 361 | ggtctgggac caagtaaagg cttccaaccc tgacctaaag ttgtgggaga ttggcaagat |
| 421 | tattggtggc atgtggcgag atctcactga tgaagaaaaa caagaatatt taaacgaata |
| 481 | cgaagcagaa aagatagagt acaatgaatc tatgaaggcc tatcataatt cccccgcgta |
| 541 | ccttgcttac ataaatgcaa aaagtcgtgc agaagctgct ttagaggaag aaagtcgaca |
| 601 | gagacaatct cgcatggaga aaggagaacc gtacatgagc attcagcctg ctgaagatcc |
| 661 | agatgattat gatgatggct tttcaatgaa gcatacagcc accgcccgtt tccagagaaa |
| 721 | ccaccgcctc atcagtgaaa ttcttagtga gagtgtggtg ccagacgttc ggtcagttgt |
| 781 | cacaacagct agaatgcagg tcctcaaacg gcaggtccag tccttaatgg ttcatcagcg |
| 841 | aaaactagaa gctgaacttc ttcaaataga ggaacgacac caggagaaga agaggaaatt |
| 901 | cctggaaagc acagattcat ttaacaatga acttaaaagg ttgtgcggtc tgaaagtaga |
| 961 | agtggatatg gagaaaattg cagctgagat tgcacaggca gaggaacagg cccgcaaaag |
| 1021 | gcaggaggaa agggagaagg aggccgcaga gcaagctgag cgcagtcaga gcagcatcgt |
| 1081 | tcctgaggaa gaacaagcag ctaacaaagg cgaggagaag aaagacgacg agaacattcc |
| 1141 | gatggagaca gaggagacac accttgaaga aacaacagag agccaacaga atggtgaaga |
| 1201 | aggcacgtct actcctgagg acaaggagag tgggcaggag ggggtcgaca gtatggcaga |
| 1261 | ggaaggaacc agtgatagta acactggctc ggagagcaac agtgcaacag tggaggagcc |
| 1321 | accaacagat cccataccag aagatgagaa aaaagaataa gtgttgcctt gttttgtgtg |
| 1381 | ttctaaatac tttttttaat gaaaaaatgt tttttggttt taatggtgtt acgtggtttg |
| 1441 | tgtattaatt ttttttcttg tccatatcac accaccaaag gcttttggac catttagcat |
| 1501 | catgagccta atggctcagt cagtcacctt tcttaagtgt tgtgaagatg gctcttttct |
| 1561 | ttggatcttg tttctagccc tcaactgctg aaagcctcag aatttagatt aattgagaaa |
| 1621 | acacccacct cttttagaga attatccttt gatgctgcag aatctactct tacaatgcct |
| 1681 | tcctacagct cactggggtg cttaccaaag ccatagcttt aaaccttccc agtccccatc |
| 1741 | agtagcttcc tgaaagtctc ctctcttgtt tacttctgca aagggtagct tcttaaaaac |
| 1801 | gtgatcatgt atgagtatgt atttgttcac ttaccctttt ttacttttaa tcaatgtcag |
| 1861 | ataccaagag ttgtgttaag ctgagtgtag tgtgtaacta actacacttg gatcttactg |
| 1921 | atccagaaat agtccccata gttagagtag ttacttatga agtggttatt aaagtgaaca |
| 1981 | cagcacatat acattatcta tactgctttt tgttatgatt aatactgggt atgttctggt |
| 2041 | aaatccatcc ttattgtata gaaaaaaaat tactttttta ccaggttttc caaagacaga |
| 2101 | atagatcaca aagctcaagg aatttaatat tcttgtaatg gactagataa ttcaaactga |
| 2161 | ttagcccatt ccagaagaaa aacagctggg aattaagtta atccacttga aattgtttta |
| 2221 | caataatcag aacatccaaa cctcaaggct caggatccca tagaccagag cccacctttt |
| 2281 | tgataaactt agtaaagtct tggagactag aagcaagata gtttgtgaca cataagcttc |
| 2341 | ccaaaaacta gaatagattt ttactgaata gtggtatatc tgatggtata tgtttcttaa |
| 2401 | aggtccaaat gtaataaaaa aaaaa |

SEQ ID NO: 102 human SMARCE1 Amino Acid Sequence (NP_003070.3)

| | |
|---|---|
| 1 | mskrpsyapp ptpapatqmp stpgfvgynp yshlaynnyr lggnpgtnsr vtassgitip |
| 61 | kppkppdkpl mpymrysrkv wdqvkasnpd lklweigkii ggmwrdltde ekqeylneye |
| 121 | aekieynesm kayhnspayl ayinaksrae aaleeesrqr qsrmekgepy msiqpaedpd |
| 181 | dyddgfsmkh tatarfqrnh rliseilses vvpdvrsvvt tarmqvlkrq vqslmvhqrk |
| 241 | leaellqiee rhqekkrkfl estdsfnnel krlcglkvev dmekiaaeia qaeeqarkrq |
| 301 | eerekeaaeq aersqssivp eeeqaankge ekkddenipm eteethleet tesqqngeeg |
| 361 | tstpedkesg qegvdsmaee gtsdsntgse snsatveepp tdpipedekk e |

SEQ ID NO: 103 Mouse SMARCE1 cDNA Sequence (NM_020618.4, CDS: 662-
1897)

| | |
|---|---|
| 1 | ggcggaggca ggggagcccc gctgggcgcc agcaaggacc taaacgcagc gacccgggtc |
| 61 | ctccccgcct acattctcca tcttctccat tcatacgtcc atcagcggag gactgaagac |
| 121 | cagagcgaag ggaaaagcca gagtgcatgg tgtgtgggaa ctgcgtccca ccctctcccg |
| 181 | ggagaggctc cggcgagcct ttcccctccg gcgcccgcct cacgcggcgg cgcccaccgc |
| 241 | ctcagtgaag ccccgggcgc gcagtctgcg cagttcctgc cgccgggccg cgaaccaggg |
| 301 | cccgcaacgc ggcccagcct tctccgccct cctcgccgtg acgaatcggc gcccgactgg |
| 361 | gacgggatcc aaattggaag acttctgagg aaacccagga gcctgacgaa atttttttta |
| 421 | aaaatccttg gcgccctaag cctcgccgcg tgctcactgg aagggctgtt cgtctgccgg |
| 481 | gagccggccg cggccggcag acaattcccg ggagcgtgtg gaaagtgcga gcgcggaagc |
| 541 | tccggcgcga ggggCggggc gagcgcggga caaagggaag cgaagccgga gctgcgggcg |
| 601 | cctgctcggc ccgcggtgtc tcagattcat tcttaaggaa ctgagaactt aatcttccaa |
| 661 | aatgtcaaaa agaccatctt atgccccacc tcccacccca gctcctgcaa cacaaatgcc |
| 721 | cagcacacca gggtttgtgg gatacaatcc atacagtcat ctcgcctaca acaactacag |
| 781 | gctgggaggg aacccgggca ccaacagccg ggtcacggcg tcctctggca ttacgattcc |
| 841 | aaagcctcca aagccaccag ataagccgct gatgccctac atgaggtaca gcagaaaggt |
| 901 | ctgggaccaa gtaaaggctt ccaaccctga cctaaagttg tgggagattg gcaagattat |
| 961 | tggtggcatg tggcgagatc tcactgatga agagaagcaa gaatatttaa acgaatacga |
| 1021 | agcagaaaag atagagtaca atgagtctat gaaggcctac cataattccc ctgcgtacct |
| 1081 | tgcctatatt aatgcaaaaa gtcgtgcgga agctgcatta gaggaagaaa gtcgacagag |
| 1141 | acagtctcgc atggagaaag gagaacctta catgagcatt cagcctgctg aggatccaga |
| 1201 | cgactatgat gatggctttt caatgaagca cacagccact gcccgtttcc agagaaacca |
| 1261 | ccgtctcatc agtgagatcc tcagtgagag tgtggtacct gatgtgcggt cggttgtcac |
| 1321 | aacagctaga atgcaggtcc tcaagcgaca ggtccagtct ttaatggttc atcagcggaa |
| 1381 | actagaagcc gagctccttc agatagagga acgacaccag gaaaagaaga ggaaattcct |
| 1441 | ggaaagcacg gactccttta acaatgaact taaaaggtta tgtggtctga aggtggaagt |
| 1501 | agacatggag aagattgcgg ctgagatcgc acaggcggag gaacaagccc gcaagaggca |
| 1561 | agaggagagg gagaaggagg cagcagagca ggctgagcgc agtcagagca gcatggcccc |
| 1621 | tgaggaagag caagtggcga acaaagccga ggagaagaaa gatgaggaga gcatcccgat |
| 1681 | ggagacagag gagacacacc ttgaagacac agcagagagc cagcagaatg gtgaagaagg |
| 1741 | cacgtctact cctgaggaca aggagagtgg gcaggagggg gttgacagca tggaggtgga |
| 1801 | agggaccagt gacagtaaca cgggctcaga gagcaacagc gccacagtgg aggagccgcc |
| 1861 | cacagaccca gtgccagaag acgagaagaa ggagtaaatg ttgccttgtt ttatgtgacc |
| 1921 | taaaactttt ttaaatgaaa aaaaaatgtg gttttttttt tggttttaat ggtgttatgt |
| 1981 | ggtctgtgta ttaattattt acttttccgt tgatacaaca tgaaggtctt tgaaccctca |
| 2041 | gcatcatagc ctaatgccag ccgctcacct ttcttagctc tcaacgtctg aaacctcaga |
| 2101 | gctgagatta atcaagacac ccatcattct ctgagaacta ccttggctgc tgcagaatcg |
| 2161 | actcttccaa atacctgcct tcagctcacg tggtgctcac caaagccata gctttaaacc |
| 2221 | cttccagccc atccacagct ttcccagtcc ctgtcttgtg tacttacaca gagtgccctc |
| 2281 | ttgaaatcat gagggggtct cttcactcac cctttctatg tcccatgtca gacaccagga |
| 2341 | gttctcttac agggtagggt gtagccagaa actggtgaga cacagatcac agagatgcct |
| 2401 | ctgggggcac tgggggtggg ggagcagggg gagtacagtt gttctttctg tggattcctt |
| 2461 | gttggtgaga gctgcgcctg cttatctaga gtgctgttca gtgtagtcga tctgggatgt |
| 2521 | gttctgggaa attcatcctt tttgtacagg ggaaagaaac actttttttt accagattgg |
| 2581 | ctttccaaag acacgataga tggcagagct taaggaatgg aatgttctta taatggacta |
| 2641 | cagacttcaa agtgattggc ccattccaaa aggaaaatgg gaatgctgtt catccatgtg |
| 2701 | agcatacttc acagtgatga aaacctcaag actcgagatc ccatagatca gagccgaacc |
| 2761 | tacttttttg ataacccctg tagtggtctt agagactaga aacaagatag tttgtagtgt |
| 2821 | gtgctcccta aaatctagaa tagattttta ctgaatagtg gtatatatga tggtatatgt |
| 2881 | ttcttaaagg tccaaacata ataaagaaat taagacaaaa aaaaaaaaaa aaaaaaaaaa |
| 2941 | aaaaaaaaaa aaaaaaaaaa a |

SEQ ID NO: 104 Mouse SMARCE1 Amino Acid Sequence (NP_065643.1)

| | |
|---|---|
| 1 | mskrpsyapp ptpapatqmp stpgfvgynp yshlaynnyr lggnpgtnsr vtassgitip |
| 61 | kppkppdkpl mpymrysrkv wdqvkasnpd lklweigkii ggmwrdltde ekqeylneye |
| 121 | aekieynesm kayhnspayl ayinaksrae aaleeesrqr qsrmekgepy msiqpaedpd |
| 181 | dyddgfsmkh tatarfqrnh rliseilses vvpdvrsvvt tarmqvlkrq vqslmvhqrk |
| 241 | leaellqiee rhqekkrkfl estdsfnnel krlcglkvev dmekiaaeia qaeeqarkrq |
| 301 | eerekeaaeq aersqssmap eeeqvankae ekkdeesipm eteethledt aesqqngeeg |
| 361 | tstpedkesg qegvdsmeve gtsdsntgse snsatveepp tdpvpedekk e |

SEQ ID NO: 105 Human DPF1 cDNA Sequence Variant 1 (NM_001135155.2, CDS:
28-1272)

| | |
|---|---|
| 1 | gtgctcccgc cccccgggaa tgaatggatg ggcggcctca gcgcccgccc gaccgctggg |
| 61 | aggaccgacc cggcggggac ctgctggggg caggacccgg ggagcaagat ggccactgtc |
| 121 | atccctggcc ccctgagcct aggcgaggac ttctaccgcg aggccatcga gcactgccgc |
| 181 | agttacaacg cgcgcctgtg cgccgagcgc agcctgcgac tgcccttcct cgactcgcag |
| 241 | accggcgtgg cccagaacaa ctgctacatc tggatggaga agacccaccg cgggccgggt |
| 301 | ttggccccgg gacagattta cacgtacccc gcccgctgtt ggaggaagaa acggagactc |
| 361 | aacatcctgg aggaccccag actcaggccc tgcgagtaca agatcgactg tgaagcaccc |
| 421 | ctgaagaagg agggtggcct cccggaaggg ccggtcctcg aggctctact gtgtgcagag |
| 481 | acgggggaga agaagattga gctgaaggag gaggagacca ttatggactg tcagaaacag |
| 541 | cagttgctgg agtttccgca tgacctcgag gtggaagact tggaggatga cattcccagg |
| 601 | aggaagaaca gggccaaagg aaaggcatat ggcatcgggg gtctccggaa acgccaggac |
| 661 | accgcttccc tggaggaccg agacaagccg tatgtctgtg atatctgtgg gaaacggtat |
| 721 | aagaaccggc cggggctcag ctaccactac acccacaccc acctggccga ggaggagggg |
| 781 | gaggagaacg ccgaacgcca cgccctgccc ttccaccgga aaaacaacca taaacagttt |
| 841 | tacaaagaat tggcctgggt ccctgaggca caaaggaaac acacagccaa gaaggcgccc |
| 901 | gacggcactg tcatccccaa cggctactgt gacttctgcc tggggggctc caagaagacg |
| 961 | gggtgtcccg aggacctcat ctcctgtgcg gactgtgggc gatcaggaca cccctcgtgt |
| 1021 | ttacaattca cggtgaacat gacggcagcc gtgcggacct accgctggca gtgcatcgag |
| 1081 | tgcaaatcct gcagcctgtg cggaacctcc gagaacgacg accagctgct gttttgtgat |
| 1141 | gactgcgatc ggggttacca catgtactgc ctgagtcccc ccatggcgga gcccccggaa |
| 1201 | gggagctgga gctgtcacct ctgtctccgg cacctgaagg aaaaggcttc tgcttacatc |
| 1261 | accctcacct aggccggctc ggctcgccgc gactctgggg tggtgctcgc ctacctgcct |
| 1321 | ctccgagctc ctcaattctc ccccaccctg aacatcccgc agggggaggg ggagaggggg |
| 1381 | aagccgagag ggggctgggc caccccctcc cctctgtgca agtggaatgt ctgccctgtg |
| 1441 | ggtgggtggg cccggccagg gcctctccct ccctccctcc ctctctgtcc cttggcaaat |
| 1501 | ggacaccagg ggcttctccc ctcaaagcca taccccgcct ctgggcgggc atggggggtg |
| 1561 | gtgggtgcca gccaggggca tggacagagc ctttttctaa agaaaaagac aaaaagttaa |
| 1621 | aaaaaaaaaa aagaagaaaa gaaaagaagt taatatatac aaagagtcct ccaaggcctg |
| 1681 | gctgggtgga ggggcgctgc tgagagtgtc caccgggcac ccgcctctgc cggccccccg |
| 1741 | ccgggcgccc caaccccaat ttctggagct gcagccgtcc cgcgccccac ccaaggtggg |
| 1801 | cgccttcccc tcttgtgccc agggcggtgg gcgtggtgtc cacccgcccc tcctggtgcc |
| 1861 | cacggtggat actgcatgat gtgaaccttg gttttgaact ctgttcctgc ccctccccga |
| 1921 | ccgccccagc ctgtgcccgc cccgtgcctg ccgtggctgg tgggtggcgg tggtggggcc |
| 1981 | gggtgggccc ccgcccagcg cctgctggaa tgagaagcac agactccgcc acggactcct |
| 2041 | tttctctccc tcctcccgcc ccgccaggcc tggcggcccc cgcccccctc gctggccatt |
| 2101 | ttgggggagt gagggggcgt ggttgtttct tgtggttgtg tgtgtttgtt gttcgggttt |
| 2161 | taaaaaaggg aaactgagac tgcaggtggg ggaggtggtg ggttttgggg ggatgtcccc |
| 2221 | taatccagga gtgccccctc acttgtcacc gagtctcctc tattgcctgc ctctgctgtg |
| 2281 | aattaacttg ttctgtgtat taaactgggc ctgacccctc tgcccacgaa aaaaaaaaaa |
| 2341 | aaaaaaaa |

SEQ ID NO: 106 Human DPF1 Amino Acid Sequence Isoform A (NP_001128627.1)

| | |
|---|---|
| 1 | mgglsarpta grtdpagtcw gqdpgskmat vipgplslge dfyreaiehc rsynarlcae |
| 61 | rslrlpflds qtgvaqnncy iwmekthrgp glapgqiyty parcwrkkrr lniledprlr |
| 121 | pceykidcea plkkegglpe gpvleallca etgekkielk eeetimdcqk qqllefphdl |
| 181 | evedleddip rrknrakgka ygigglrkrq dtasledrdk pyvcdicgkr yknrpglsyh |
| 241 | yththlaeee geenaerhal pfhrknnhkq fykelawvpe aqrkhtakka pdgtvipngy |
| 301 | cdfclggskk tgcpedlisc adcgrsghps clqftvnmta avrtyrwqci eckscslcgt |
| 361 | senddqllfc ddcdrgyhmy clsppmaepp egswschlcl rhlkekasay itlt |

SEQ ID NO: 107 Human DPF1 cDNA Sequence Variant 2 (NM_004647.3, CDS: 28-
1170)

| | |
|---|---|
| 1 | gtgctcccgc cccccgggaa tgaatggatg ggcggcctca gcgcccgccc gaccgctggg |
| 61 | aggaccgacc cggcggggac ctgctggggg caggacccgg ggagcaagat ggccactgtc |
| 121 | atccctggcc ccctgagcct aggcgaggac ttctaccgcg aggccatcga gcactgccgc |
| 181 | agttacaacg cgcgcctgtg cgccgagcgc agcctgcgac tgcccttcct cgactcgcag |
| 241 | accggcgtgg cccagaacaa ctgctacatc tggatggaga agacccaccg cgggccgggt |
| 301 | ttggccccgg gacagattta cacgtacccc gcccgctgtt ggaggaagaa acggagactc |
| 361 | aacatcctgg aggaccccag actcaggccc tgcgagtaca agatcgactg tgaagcaccc |
| 421 | ctgaagaagg agggtggcct cccggaaggg ccggtcctcg aggctctact gtgtgcagag |
| 481 | acgggggaga agaagattga gctgaaggag gaggagacca ttatggactg tcagaaacag |
| 541 | cagttgctgg agtttccgca tgacctcgag gtggaagact tggaggatga cattcccagg |
| 601 | aggaagaaca gggccaaagg aaaggcatat ggcatcgggg gtctccggaa acgccaggac |
| 661 | accgcttccc tggaggaccg agacaagccg tatgtctgtg ataagtttta caaagaattg |
| 721 | gcctgggtcc ctgaggcaca aaggaaacac acagccaaga aggcgcccga cggcactgtc |
| 781 | atccccaacg gctactgtga cttctgcctg gggggctcca agaagacggg gtgtcccgag |
| 841 | gacctcatct cctgtgcgga ctgtgggcga tcaggacacc cctcgtgttt acaattcacg |
| 901 | gtgaacatga cggcagccgt gcggacctac cgctggcagt gcatcgagtg caaatcctgc |
| 961 | agcctgtgcg gaacctccga gaacgacggt gccagctggg cgggtctcac cccccaggac |
| 1021 | cagctgctgt tttgtgatga ctgcgatcgg ggttaccaca tgtactgcct gagtcccccc |
| 1081 | atggcggagc ccccggaagg gagctggagc tgtcacctct gtctccggca cctgaaggaa |
| 1141 | aaggcttctg cttacatcac cctcacctag gccggctcgg ctcgccgcga ctctggggtg |
| 1201 | gtgctcgcct acctgcctct ccgagctcct caattctccc ccaccctgaa catcccgcag |
| 1261 | ggggaggggg agagggggaa gccgagaggg ggctgggcca ccccctcccc tctgtgcaag |
| 1321 | tggaatgtct gccctgtggg tgggtgggcc cggccagggc ctctccctcc ctccctccct |
| 1381 | ctctgtccct tggcaaatgg acaccagggg cttctcccct caaagccata ccccgcctct |
| 1441 | gggcgggcat ggggggtggt gggtgccagc caggggcatg gacagagcct ttttctaaag |
| 1501 | aaaaagacaa aaagttaaaa aaaaaaaaaa gaagaaaaga aaagaagtta atatatacaa |
| 1561 | agagtcctcc aaggcctggc tgggtggagg ggcgctgctg agagtgtcca ccgggcaccc |
| 1621 | gcctctgccg gccccccgcc gggcgcccca accccaattt ctggagctgc agccgtcccg |
| 1681 | cgccccaccc aaggtgggcg ccttcccctc ttgtgcccag ggcggtgggc gtggtgtcca |
| 1741 | cccgcccctc ctggtgccca cggtggatac tgcatgatgt gaaccttggt tttgaactct |
| 1801 | gttcctgccc ctccccgacc gccccagcct gtgcccgccc cgtgcctgcc gtggctggtg |
| 1861 | ggtggcggtg gtggggccgg gtgggccccc gcccagcgcc tgctggaatg agaagcacag |
| 1921 | actccgccac ggactccttt tctctccctc ctcccgcccc gccaggcctg gcggcccccg |
| 1981 | cccccctcgc tggccatttt gggggagtga gggggcgtgg ttgtttcttg tggttgtgtg |
| 2041 | tgtttgttgt tcgggtttta aaaaagggaa actgagactg caggtggggg aggtggtggg |
| 2101 | ttttgggggg atgtccccta atccaggagt gccccctcac ttgtcaccga gtctcctcta |
| 2161 | ttgcctgcct ctgctgtgaa ttaacttgtt ctgtgtatta aactgggcct gacccctctg |
| 2221 | cccacgaaaa aaaaaaaaaa aaaaaa |

SEQ ID NO: 108 Human DPF1 Amino Acid Sequence Isoform B (NP_004638.2)

| | |
|---|---|
| 1 | mgglsarpta grtdpagtcw gqdpgskmat vipgplslge dfyreaiehc rsynarlcae |
| 61 | rslrlpflds qtgvaqnncy iwmekthrgp glapgqiyty parcwrkkrr lniledprlr |
| 121 | pceykidcea plkkegglpe gpvleallca etgekkielk eeetimdcqk qqllefphdl |
| 181 | evedleddip rrknrakgka ygigglrkrq dtasledrdk pyvcdkfyke lawvpeaqrk |
| 241 | htakkapdgt vipngycdfc lggskktgcp edliscadcg rsghpsclqf tvnmtaavrt |
| 301 | yrwqciecks cslcgtsend gaswagltpq dqllfcddcd rgyhmyclsp pmaeppegsw |
| 361 | schlclrhlk ekasayitlt |

SEQ ID NO: 109 Human DPF1 cDNA Sequence Variant 3 (NM_001135156.2, CDS:
288-1286)

| | |
|---|---|
| 1 | cgcagcccca agaatgaatg aaatcgtagc gcgctgggcg gcagagcggg cggcgcaggc |
| 61 | cgggctgggc ccgcgcgcgg cggcagcggc gccccgggcc ggaggcggcc cagccgagcg |
| 121 | ggccatggcc accgccattc agaacccgct caagtcgcga ggacttctac cgcgaggcca |
| 181 | tcgagcactg ccgcagttac aacgcgcgcc tgtgcgccga gcgcagcctg cgactgccct |
| 241 | tcctcgactc gcagaccggc gtggcccaga acaactgcta catctggatg gagaagaccc |
| 301 | accgcgggcc gggtttggcc ccgggacaga tttacacgta ccccgcccgc tgttggagga |
| 361 | agaaacggag actcaacatc ctggaggacc ccagactcag gccctgcgag tacaagatcg |
| 421 | actgtgaagc acccctgaag aaggagggtg gcctcccgga agggccggtc ctcgaggctc |
| 481 | tactgtgtgc agagacgggg gagaagaaga ttgagctgaa ggaggaggag accattatgg |
| 541 | actgtcagaa acagcagttg ctggagtttc cgcatgacct cgaggtggaa gacttggagg |
| 601 | atgacattcc caggaggaag aacagggcca aaggaaaggc atatggcatc gggggtctcc |
| 661 | ggaaacgcca ggacaccgct tccctggagg accgagacaa gccgtatgtc tgtgatatct |
| 721 | gtgggaaacg gtataagaac cggccggggc tcagctacca ctacacccac acccacctgg |
| 781 | ccgaggagga gggggaggag aacgccgaac gccacgccct gcccttccac cggaaaaaca |
| 841 | accataaaca gttttacaaa gaattggcct gggtccctga ggcacaaagg aaacacacag |
| 901 | ccaagaaggc gcccgacggc actgtcatcc ccaacggcta ctgtgacttc tgcctggggg |
| 961 | gctccaagaa gacggggtgt cccgaggacc tcatctcctg tgcggactgt gggcgatcag |
| 1021 | gacacccctc gtgtttacaa ttcacggtga acatgacggc agccgtgcgg acctaccgct |
| 1081 | ggcagtgcat cgagtgcaaa tcctgcagcc tgtgcggaac ctccgagaac gacgaccagc |
| 1141 | tgctgttttg tgatgactgc gatcggggtt accacatgta ctgcctgagt ccccccatgg |
| 1201 | cggagccccc ggaagggagc tggagctgtc acctctgtct ccggcacctg aaggaaaagg |
| 1261 | cttctgctta catcaccctc acctaggccg gctcggctcg ccgcgactct ggggtggtgc |
| 1321 | tcgcctacct gcctctccga gctcctcaat tctcccccac cctgaacatc ccgcaggggg |
| 1381 | agggggagag ggggaagccg agagggggct gggccacccc ctcccctctg tgcaagtgga |
| 1441 | atgtctgccc tgtgggtggg tgggcccggc cagggcctct ccctccctcc ctccctctct |
| 1501 | gtcccttggc aaatggacac caggggcttc tcccctcaaa gccatacccc gcctctgggc |
| 1561 | gggcatgggg ggtggtgggt gccagccagg ggcatggaca gagccttttt ctaaagaaaa |
| 1621 | agacaaaaag ttaaaaaaaa aaaaaagaag aaaagaaaag aagttaatat atacaaagag |
| 1681 | tcctccaagg cctggctggg tggaggggcg ctgctgagag tgtccaccgg gcacccgcct |
| 1741 | ctgccggccc cccgccgggc gccccaaccc caatttctgg agctgcagcc gtcccgcgcc |
| 1801 | ccacccaagg tgggcgcctt cccctcttgt gcccagggcg gtgggcgtgg tgtccacccg |
| 1861 | cccctcctgg tgcccacggt ggatactgca tgatgtgaac cttggttttg aactctgttc |
| 1921 | ctgcccctcc ccgaccgccc cagcctgtgc ccgccccgtg cctgccgtgg ctggtgggtg |
| 1981 | gcggtggtgg ggccgggtgg gcccccgccc agcgcctgct ggaatgagaa gcacagactc |
| 2041 | cgccacggac tccttttctc tccctcctcc cgccccgcca ggcctggcgg cccccgcccc |
| 2101 | cctcgctggc cattttgggg gagtgagggg gcgtggttgt ttcttgtggt tgtgtgtgtt |
| 2161 | tgttgttcgg gttttaaaaa agggaaactg agactgcagg tgggggaggt ggtgggtttt |
| 2221 | ggggggatgt cccctaatcc aggagtgccc cctcacttgt caccgagtct cctctattgc |
| 2281 | ctgcctctgc tgtgaattaa cttgttctgt gtattaaact gggcctgacc cctctgccca |
| 2341 | cgaaaaaaaa aaaaaaaaaa aa |

SEQ ID NO: 110 Human DPF1 Amino Acid Sequence Isoform C (NP_001128628.1)

| | |
|---|---|
| 1 | mekthrgpgl apgqiytypa rcwrkkrrln iledprlrpc eykidceapl kkegglpegp |
| 61 | vleallcaet gekkielkee etimdcqkqq llefphdlev edleddiprr knrakgkayg |
| 121 | igglrkrqdt asledrdkpy vcdicgkryk nrpglsyhyt hthlaeeege enaerhalpf |
| 181 | hrknnhkqfy kelawvpeaq rkhtakkapd gtvipngycd fclggskktg cpedliscad |
| 241 | cgrsghpscl qftvnmtaav rtyrwqciec kscslcgtse nddqllfcdd cdrgyhmycl |
| 301 | sppmaeppeg swschlclrh lkekasayit lt |

SEQ ID NO: 111 Human DPF1 cDNA Sequence Variant 4 (NM_001289978.1, CDS:
28-1302)

| | |
|---|---|
| 1 | gtgctcccgc cccccgggaa tgaatggatg ggcggcctca gcgcccgccc gaccgctggg |
| 61 | aggaccgacc cggcggggac ctgctggggg caggacccgg ggagcaagat ggccactgtc |
| 121 | atccctggcc ccctgagcct aggcgaggac ttctaccgcg aggccatcga gcactgccgc |
| 181 | agttacaacg cgcgcctgtg cgccgagcgc agcctgcgac tgcccttcct cgactcgcag |
| 241 | accggcgtgg cccagaacaa ctgctacatc tggatggaga agacccaccg cgggccgggt |
| 301 | ttggccccgg gacagattta cacgtacccc gcccgctgtt ggaggaagaa acggagactc |
| 361 | aacatcctgg aggaccccag actcaggccc tgcgagtaca agatcgactg tgaagcaccc |
| 421 | ctgaagaagg agggtggcct cccggaaggg ccggtcctcg aggctctact gtgtgcagag |
| 481 | acgggggaga agaagattga gctgaaggag gaggagacca ttatggactg tcagaaacag |
| 541 | cagttgctgg agtttccgca tgacctcgag gtggaagact tggaggatga cattcccagg |
| 601 | aggaagaaca gggccaaagg aaaggcatat ggcatcgggg gtctccggaa acgccaggac |
| 661 | accgcttccc tggaggaccg agacaagccg tatgtctgtg atatctgtgg gaaacggtat |
| 721 | aagaaccggc cggggctcag ctaccactac acccacaccc acctggccga ggaggagggg |
| 781 | gaggagaacg ccgaacgcca cgccctgccc ttccaccgga aaaacaacca taaacagttt |
| 841 | tacaaagaat tggcctgggt ccctgaggca caaaggaaac acacagccaa gaaggcgccc |
| 901 | gacggcactg tcatccccaa cggctactgt gacttctgcc tggggggctc caagaagacg |
| 961 | gggtgtcccg aggacctcat ctcctgtgcg gactgtgggc gatcaggaca cccctcgtgt |
| 1021 | ttacaattca cggtgaacat gacggcagcc gtgcggacct accgctggca gtgcatcgag |
| 1081 | tgcaaatcct gcagcctgtg cggaacctcc gagaacgacg gtgccagctg ggcgggtctc |
| 1141 | accccccagg accagctgct gttttgtgat gactgcgatc ggggttacca catgtactgc |
| 1201 | ctgagtcccc ccatggcgga gcccccggaa gggagctgga gctgtcacct ctgtctccgg |
| 1261 | cacctgaagg aaaaggcttc tgcttacatc accctcacct aggccggctc ggctcgccgc |
| 1321 | gactctgggg tggtgctcgc ctacctgcct ctccgagctc ctcaattctc ccccaccctg |
| 1381 | aacatcccgc agggggaggg ggagaggggg aagccgagag ggggctgggc caccccctcc |
| 1441 | cctctgtgca agtggaatgt ctgccctgtg ggtgggtggg cccggccagg gcctctccct |
| 1501 | ccctccctcc ctctctgtcc cttggcaaat ggacaccagg ggcttctccc ctcaaagcca |
| 1561 | taccccgcct ctgggcgggc atggggggtg gtgggtgcca gccaggggca tggacagagc |
| 1621 | ctttttctaa agaaaaagac aaaaagttaa aaaaaaaaaa aagaagaaaa gaaaagaagt |
| 1681 | taatatatac aaagagtcct ccaaggcctg gctgggtgga ggggcgctgc tgagagtgtc |
| 1741 | caccgggcac ccgcctctgc cggccccccg ccgggcgccc caaccccaat ttctggagct |
| 1801 | gcagccgtcc cgcgccccac ccaaggtggg cgccttcccc tcttgtgccc agggcggtgg |
| 1861 | gcgtggtgtc cacccgcccc tcctggtgcc cacggtggat actgcatgat gtgaaccttg |
| 1921 | gttttgaact ctgttcctgc ccctccccga ccgccccagc ctgtgcccgc cccgtgcctg |
| 1981 | ccgtggctgg tgggtggcgg tggtggggcc gggtgggccc ccgcccagcg cctgctggaa |
| 2041 | tgagaagcac agactccgcc acggactcct tttctctccc tcctcccgcc ccgccaggcc |
| 2101 | tggcggcccc cgcccccctc gctggccatt ttgggggagt gagggggcgt ggttgtttct |
| 2161 | tgtggttgtg tgtgtttgtt gttcgggttt taaaaaaggg aaactgagac tgcaggtggg |
| 2221 | ggaggtggtg ggttttgggg ggatgtcccc taatccagga gtgccccctc acttgtcacc |
| 2281 | gagtctcctc tattgcctgc ctctgctgtg aattaacttg ttctgtgtat taaactgggc |
| 2341 | ctgacccctc tgcccacgaa aaaaaaaaaa aaaaaaaa |

SEQ ID NO: 112 Human DPF1 Amino Acid Sequence Isoform D (NP_001276907.1)

| | |
|---|---|
| 1 | mgglsarpta grtdpagtcw gqdpgskmat vipgplslge dfyreaiehc rsynarlcae |
| 61 | rslrlpflds qtgvaqnncy iwmekthrgp glapgqiyty parcwrkkrr lniledprlr |
| 121 | pceykidcea plkkegglpe gpvleallca etgekkielk eeetimdcqk qqllefphdl |
| 181 | evedleddip rrknrakgka ygigglrkrq dtasledrdk pyvcdicgkr yknrpglsyh |
| 241 | yththlaeee geenaerhal pfhrknnhkq fykelawvpe aqrkhtakka pdgtvipngy |
| 301 | cdfclggskk tgcpedlisc adcgrsghps clqftvnmta avrtyrwqci eckscslcgt |
| 361 | sendgaswag ltpqdqllfc ddcdrgyhmy clsppmaepp egswschlcl rhlkekasay |
| 421 | itlt |

SEQ ID NO: 113 Human DPF1 cDNA Sequence Variant 5 (NM_001363579.1, CDS:
106-1272)

| | |
|---|---|
| 1 | gaaatcgtag cgcgctgggc ggcagagcgg gcggcgcagg ccgggctggg cccgcgcgcg |
| 61 | gcggcagcgg cgccccgggc cggaggcggc ccagccgagc gggccatggc caccgccatt |
| 121 | cagaacccgc tcaagtccct aggcgaggac ttctaccgcg aggccatcga gcactgccgc |
| 181 | agttacaacg cgcgcctgtg cgccgagcgc agcctgcgac tgcccttcct cgactcgcag |
| 241 | accggcgtgg cccagaacaa ctgctacatc tggatggaga agacccaccg cgggccgggt |
| 301 | ttggccccgg gacagattta cacgtacccc gcccgctgtt ggaggaagaa acggagactc |
| 361 | aacatcctgg aggaccccag actcaggccc tgcgagtaca agatcgactg tgaagcaccc |
| 421 | ctgaagaagg agggtggcct cccggaaggg ccggtcctcg aggctctact gtgtgcagag |
| 481 | acgggggaga agaagattga gctgaaggag gaggagacca ttatggactg tcagaaacag |
| 541 | cagttgctgg agtttccgca tgacctcgag gtggaagact tggaggatga cattcccagg |
| 601 | aggaagaaca gggccaaagg aaaggcatat ggcatcgggg gtctccggaa acgccaggac |
| 661 | accgcttccc tggaggaccg agacaagccg tatgtctgtg atatctgtgg gaaacggtat |
| 721 | aagaaccggc cggggctcag ctaccactac acccacaccc acctggccga ggaggagggg |
| 781 | gaggagaacg ccgaacgcca cgccctgccc ttccaccgga aaaacaacca taaacagttt |
| 841 | tacaaagaat tggcctgggt ccctgaggca caaaggaaac acacagccaa gaaggcgccc |
| 901 | gacggcactg tcatccccaa cggctactgt gacttctgcc tggggggctc caagaagacg |
| 961 | gggtgtcccg aggacctcat ctcctgtgcg gactgtgggc gatcaggaca cccctcgtgt |
| 1021 | ttacaattca cggtgaacat gacggcagcc gtgcggacct accgctggca gtgcatcgag |
| 1081 | tgcaaatcct gcagcctgtg cggaacctcc gagaacgacg accagctgct gttttgtgat |
| 1141 | gactgcgatc ggggttacca catgtactgc ctgagtcccc ccatggcgga gcccccggaa |
| 1201 | gggagctgga gctgtcacct ctgtctccgg cacctgaagg aaaaggcttc tgcttacatc |
| 1261 | accctcacct aggccggctc ggctcgccgc gactctgggg tggtgctcgc ctacctgcct |
| 1321 | ctccgagctc ctcaattctc ccccaccctg aacatcccgc agggggaggg ggagaggggg |
| 1381 | aagccgagag ggggctgggc caccccctcc cctctgtgca agtggaatgt ctgccctgtg |
| 1441 | ggtgggtggg cccggccagg gcctctccct ccctccctcc ctctctgtcc cttggcaaat |
| 1501 | ggacaccagg ggcttctccc ctcaaagcca taccccgcct ctgggcgggc atggggggtg |
| 1561 | gtgggtgcca gccaggggca tggacagagc ctttttctaa agaaaaagac aaaaagttaa |
| 1621 | aaaaaaaaaa aagaagaaaa gaaaagaagt taatatatac aaagagtcct ccaaggcctg |
| 1681 | gctgggtgga ggggcgctgc tgagagtgtc caccgggcac ccgcctctgc cggccccccg |
| 1741 | ccgggcgccc caaccccaat ttctggagct gcagccgtcc cgcgccccac ccaaggtggg |
| 1801 | cgccttcccc tcttgtgccc agggcggtgg gcgtggtgtc cacccgcccc tcctggtgcc |
| 1861 | cacggtggat actgcatgat gtgaaccttg gttttgaact ctgttcctgc ccctccccga |
| 1921 | ccgccccagc ctgtgcccgc cccgtgcctg ccgtggctgg tgggtggcgg tggtggggcc |
| 1981 | gggtgggccc ccgcccagcg cctgctggaa tgagaagcac agactccgcc acggactcct |
| 2041 | tttctctccc tcctcccgcc ccgccaggcc tggcggcccc cgcccccctc gctggccatt |
| 2101 | ttgggggagt gagggggcgt ggttgtttct tgtggttgtg tgtgtttgtt gttcgggttt |
| 2161 | taaaaaaggg aaactgagac tgcaggtggg ggaggtggtg ggttttgggg ggatgtcccc |
| 2221 | taatccagga gtgccccctc acttgtcacc gagtctcctc tattgcctgc ctctgctgtg |
| 2281 | aattaacttg ttctgtgtat taaactgggc ctgacccctc tgcccacga |

SEQ ID NO: 114 Human DPF1 Amino Acid Sequence Isoform E (NP_001350508.1)

| | |
|---|---|
| 1 | mataiqnplk slgedfyrea iehcrsynar lcaerslrlp fldsqtgvaq nncyiwmekt |
| 61 | hrgpglapgq iytyparcwr kkrrlniled prlrpceyki dceaplkkeg glpegpvlea |
| 121 | llcaetgekk ielkeeetim dcqkqqllef phdlevedle ddiprrknra kgkaygiggl |
| 181 | rkrqdtasle drdkpyvcdi cgkryknrpg lsyhyththl aeeegeenae rhalpfhrkn |
| 241 | nhkqfykela wvpeaqrkht akkapdgtvi pngycdfclg gskktgcped liscadcgrs |
| 301 | ghpsclqftv nmtaavrtyr wqcieckscs lcgtsenddq llfcddcdrg yhmyclsppm |
| 361 | aeppegswsc hlclrhlkek asayitlt |

SEQ ID NO: 115 Mouse DPF1 cDNA Sequence (NM_013874.2, CDS: 77-1243)

| | |
|---|---|
| 1 | gcaggccggg ctgggcccgc gctcagcggc agcagcagcg gcgccccggg ccggaggcgg |
| 61 | cccagccgag cgggccatgg ccaccgccat tcagaacccg ctcaagtccc ttggcgagga |
| 121 | cttctaccgg gaggccatcg agcactgtcg cagctacaac gcgcgcctgt gtgccgagcg |
| 181 | cagcctgcgc ctgcctttcc tcgactcgca gaccggagtg gcccagaaca actgctacat |
| 241 | ctggatggag aagacccacc gcgggcctgg tttggccccg ggacagatct acacttaccc |
| 301 | cgcccgctgt tggaggaaga aacggagact caacatcctg gaggacccca ggctccggcc |
| 361 | ctgcgagtac aagatcgatt gtgaggcacc tctgaagaag gagggtggcc tcccggaagg |
| 421 | gccagtcctc gaggctctgc tgtgtgctga gactggagag aagaaagtgg agctgaagga |
| 481 | ggaggagacc atcatggact gtcagaaaca gcagttgctg gagtttccgc atgatctcga |
| 541 | ggtagaagac ttggaggaag acattcccag gaggaagaac agggcaagag gaaaggcata |
| 601 | tggcattgga ggtctccgca aacgccagga caccgcatcc ctggaggacc gagacaagcc |
| 661 | gtacgtctgt gatatctgtg ggaagagata taagaaccgg ccaggactca gctaccatta |
| 721 | cacccacacc cacctggctg aggaggaggg ggaggagcac actgaacgcc acgccctgcc |
| 781 | tttccaccgg aaaaacaacc ataaacagtt ttacaaagaa ttggcctggg tccccgaggc |
| 841 | acagaggaaa cacacagcca agaaagcacc agatggcact gtcatcccca atggctactg |
| 901 | tgacttttgc ctggggggct ccaagaagac tgggtgtccc gaggacctca tctcctgtgc |
| 961 | ggactgtggg cgatcaggac atccctcgtg tttacagttc acggtgaaca tgaccgcggc |
| 1021 | tgtgcggacc taccgctggc agtgcattga atgcaagtcc tgcagcctgt gtggcacctc |
| 1081 | ggagaatgac gaccagctgc tgttctgtga tgactgcgat cgaggttacc acatgtactg |
| 1141 | cctgagccct cccatggcgg agcccccgga agggagctgg agctgccacc tctgtctccg |
| 1201 | gcacttgaag gaaaaggcct ctgcttacat caccctgacc taggcccggc tctgcttccc |
| 1261 | caggatcttt gggtggtgct atctcctgcc tcttggagct cctggcgctc cccacccggt |
| 1321 | gtccccagtg gaagggatgg ggtgaagccc agagtggggg ggggcaaggt gttctccctc |
| 1381 | tgcaagtgga atgttaccct gtgggtggct gggtccaaca gggtccctcc tgtcccccct |
| 1441 | cttcatccct tgacaaatgg gcaccaggct tctgctctcc tcaaagccat acccccgcct |
| 1501 | ttgggcgggc atagaggggt agtggatgct agccagcagc acggaaagag cctttttcta |
| 1561 | aagaaaaaga caaaacgtgg aaaaaaaagg gaaaaaaatt aatatataca aagagtccta |
| 1621 | taaagcctgg ctgggtggag aggcactgtt gagtgtctgc tggggacctg actttaccag |
| 1681 | tttcctgaat ggcgcctccc cacctcattt ctggagttgc aatggtctca actcccatct |
| 1741 | gaggtgggta ccaccccttc ctcagtaccc accgtggata ctgcatgtga actatggttt |
| 1801 | tgaactcttc ctcctcctcc ttgagagccc cgccctgcgc ccgcgtggtg cctgcctgcc |
| 1861 | aggcctgggg cgtgcagccg gggaggcggg tggggtgagg caggcaggca gccagccccc |
| 1921 | tgcagtgaga agcacagatt gcaatggact cagttttttt tttttttttt tttttttttc |
| 1981 | ctttctccct tcccacccct ttccttccct acccagccag gctgggctgc ctcctgcccc |
| 2041 | cctcgctagc catttggggg tggcaagggg gtgtggttgt ttctcgtggt tgtgtgtgtt |
| 2101 | tgttgttcgg gtttttaaaa ggggaaattg agactgcaag tgggggaggt ggagggtctg |
| 2161 | ggggagtctg cccccaatcc aggagtaccc cccttgccac caagtctcct ttattgcctg |
| 2221 | cctctgctgt gaattaactt gttctgtgta ttaaactggg cctgacccct ctgcccac |

SEQ ID NO: 116 Mouse DPF1 Amino Acid Sequence (NP_038902.1)

| | |
|---|---|
| 1 | mataiqnplk slgedfyrea iehcrsynar lcaerslrlp fldsqtgvaq nncyiwmekt |
| 61 | hrgpglapgq iytyparcwr kkrrlniled prlrpceyki dceaplkkeg glpegpvlea |
| 121 | llcaetgekk velkeeetim dcqkqqllef phdlevedle ediprrknra rgkaygiggl |
| 181 | rkrqdtasle drdkpyvcdi cgkryknrpg lsyhyththl aeeegeehte rhalpfhrkn |
| 241 | nhkqfykela wvpeaqrkht akkapdgtvi pngycdfclg gskktgcped liscadcgrs |
| 301 | ghpsclqftv nmtaavrtyr wqcieckscs lcgtsenddq llfcddcdrg yhmyclsppm |
| 361 | aeppegswsc hlclrhlkek asayitlt |

SEQ ID NO: 117 Human DPF2 cDNA Sequence Variant 1 (NM_006268.4, CDS: 134-
1309)

| | |
|---|---|
| 1 | agtgctcgct ctagtgcgcg cgcccggacg gcgcctgcgc agagggcaag gaacctggta |
| 61 | ccccggtgcg gtcccggcgc ctgcgcgctg cggactgtgg ggcttctcgg cccgaggcag |
| 121 | aggaacaggg aagatggcgg ctgtggtgga gaatgtagtg aagctccttg gggagcagta |
| 181 | ctacaaagat gccatggagc agtgccacaa ttacaatgct cgcctctgtg ctgagcgcag |
| 241 | cgtgcgcctg cctttcttgg actcacagac cggagtagcc cagagcaatt gttacatctg |
| 301 | gatggaaaag cgacaccggg gtccaggatt ggcctccgga cagctgtact cctaccctgc |
| 361 | ccggcgctgg cggaaaaagc ggcgagccca tccccctgag gatccacgac tttccttccc |
| 421 | atctattaag ccagacacag accagaccct gaagaaggag gggctgatct ctcaggatgg |
| 481 | cagtagttta gaggctctgt tgcgcactga ccccctggag aagcgaggtg ccccggatcc |
| 541 | ccgagttgat gatgacagcc tgggcgagtt tcctgtgacc aacagtcgag cgcgaaagcg |
| 601 | gatcctagaa ccagatgact tcctggatga cctcgatgat gaagactatg aagaagatac |
| 661 | tcccaagcgt cggggaaagg ggaaatccaa gggtaagggt gtgggcagtg cccgtaagaa |
| 721 | gctggatgct tccatcctgg aggaccggga taagccctat gcctgtgaca tttgtggaaa |
| 781 | acgttacaag aaccgaccag gcctcagtta ccactatgcc cactcccact tggctgagga |
| 841 | ggagggcgag gacaaggaag actctcaacc acccactcct gtttcccaga ggtctgagga |
| 901 | gcagaaatcc aaaaagggtc ctgatggatt ggccttgccc aacaactact gtgacttctg |
| 961 | cctgggggac tcaaagatta acaagaagac gggacaaccc gaggagctgg tgtcctgttc |
| 1021 | tgactgtggc cgctcagggc atccatcttg cctccaattt acccccgtga tgatggcggc |
| 1081 | agtgaagaca taccgctggc agtgcatcga gtgcaaatgt tgcaatatct gcggcacctc |
| 1141 | cgagaatgac gaccagttgc tcttctgtga tgactgcgat cgtggctacc acatgtactg |
| 1201 | tctcaccccg tccatgtctg agccccctga aggaagttgg agctgccacc tgtgtctgga |
| 1261 | cctgttgaaa gagaaagctt ccatctacca gaaccagaac tcctcttgat gtggccaccc |
| 1321 | acctgctccc cgacatatct aaggctgttt ctctcctcca cttcatattt catacccatc |
| 1381 | tttcccttct tcctcctctc cttcacaaat ccagagaacc ttggggtggt tgtgccagcc |
| 1441 | tgcctttggc agctgcaagc tgaggtggca gctctgacca cctctggccc caggccctca |
| 1501 | gggagaaagg agcaacacac tgcccctagg cgtgcgtgtg gcccagtttc tctctgctct |
| 1561 | ccattaagtg cattcactct gcttgccttg ggcccagccc ctggtgatca cagggttcaa |
| 1621 | acagtgtcct cctagaaaga gtgggagagc agctcacttc tctgtgttct gcctcccctc |
| 1681 | tggtctccag agttttcctg tcctctagag gcaagccagg ccagggagct gggagcgagc |
| 1741 | aagctgaggc cacgtccaca aggagctttt catgcccctg tgccgcatag cctcacctct |
| 1801 | ttcctccaga gtggctctct gcggccctgt gttcctgcta cagagtgttc ttttctggag |
| 1861 | tcaggatgtt ctcggtcacc ctcctggttc tgccctgtcc cattccaccc caccccaggg |
| 1921 | ggaacagtag cttcaccttg ttattcccat tgctctcctg gctcactctt acggtcggtc |
| 1981 | tccagtgact gaagcattcc ccacccttgg aatttctcat cttctgcctc ccttcctact |
| 2041 | ccttttggtt ttgtggggag aggggaagga tcagggggcc aggccagcag ctcgggggcc |
| 2101 | acaaggagat ggataatgtg cctgtttttt aacacaacaa aaaagcctac ctccaaaatc |
| 2161 | ccctttttgt tcttcctgga cctgggcatt cagcctcctg ctcttaactg aattgggagc |
| 2221 | ctctgccacc tgccccgtgt atcctggctc tcagctcatg gggaagccac atagacatcc |
| 2281 | ctttcttccc ttgcacgctc gctagcagct ggtaaggtct tcacaccctg attcctcaag |
| 2341 | ttttctgctt agtggcactg acattaagta gtggggggac agtccatgcc aggacaccct |
| 2401 | ggagtagcct tcccccttgg ccgtgggcag gccctaactc actgtcgctt tggagttgag |
| 2461 | gtgtcttttt tttttctttc tttagttcct gtattctaaa cattagtaaa aataaatgtt |
| 2521 | tttacacaga aaaaaaaaaa aaaaa |

SEQ ID NO: 118 Human DPF2 Amino Acid Sequence Isoform 1 (NP_006259.1)

| | |
|---|---|
| 1 | maavvenvvk llgeqyykda meqchnynar lcaersvrlp fldsqtgvaq sncyiwmekr |
| 61 | hrgpglasgq lysyparrwr kkrrahpped prlsfpsikp dtdqtlkkeg lisqdgssle |
| 121 | allrtdplek rgapdprvdd dslgefpvtn srarkrilep ddflddldde dyeedtpkrr |
| 181 | gkgkskgkgv gsarkkldas iledrdkpya cdicgkrykn rpglsyhyah shlaeeeged |
| 241 | kedsqpptpv sqrseeqksk kgpdglalpn nycdfclgds kinkktgqpe elvscsdcgr |
| 301 | sghpsclqft pvmmaavkty rwqcieckcc nicgtsendd qllfcddcdr gyhmycltps |
| 361 | mseppegsws chlcldllke kasiyqnqns s |

SEQ ID NO: 119 Human DPF2 cDNA Sequence Variant 2 (NM_001330308.1, CDS:
134-1351)

| | |
|---|---|
| 1 | agtgctcgct ctagtgcgcg cgcccggacg gcgcctgcgc agagggcaag gaacctggta |
| 61 | ccccggtgcg gtcccggcgc ctgcgcgctg cggactgtgg ggcttctcgg cccgaggcag |
| 121 | aggaacaggg aagatggcgg ctgtggtgga gaatgtagtg aagctccttg gggagcagta |
| 181 | ctacaaagat gccatggagc agtgccacaa ttacaatgct cgcctctgtg ctgagcgcag |
| 241 | cgtgcgcctg cctttcttgg actcacagac cggagtagcc cagagcaatt gttacatctg |
| 301 | gatggaaaag cgacaccggg gtccaggatt ggcctccgga cagctgtact cctaccctgc |
| 361 | ccggcgctgg cggaaaaagc ggcgagccca tccccctgag gatccacgac tttccttccc |
| 421 | atctattaag ccagacacag accagaccct gaagaaggag gggctgatct ctcaggatgg |
| 481 | cagtagttta gaggctctgt tgcgcactga ccccctggag aagcgaggtg ccccggatcc |
| 541 | ccgagttgat gatgacagcc tgggcgagtt tcctgtgacc aacagtcgag cgcgaaagcg |
| 601 | gatcctagaa ccagatgact tcctggatga cctcgatgat gaagactatg aagaagatac |
| 661 | tcccaagcgt cggggaaagg ggaaatccaa gggtaagggt gtgggcagtg cccgtaagaa |
| 721 | gctggatgct tccatcctgg aggaccggga taagccctat gcctgtgaca atagtttcaa |
| 781 | acaaaagcat acctcgaaag cgccccagag agtttgtgga aaacgttaca agaaccgacc |
| 841 | aggcctcagt taccactatg cccactccca cttggctgag gaggagggcg aggacaagga |
| 901 | agactctcaa ccacccactc ctgtttccca gaggtctgag gagcagaaat ccaaaaaggg |
| 961 | tcctgatgga ttggccttgc ccaacaacta ctgtgacttc tgcctggggg actcaaagat |
| 1021 | taacaagaag acgggacaac ccgaggagct ggtgtcctgt tctgactgtg gccgctcagg |
| 1081 | gcatccatct tgcctccaat ttacccccgt gatgatggcg gcagtgaaga cataccgctg |
| 1141 | gcagtgcatc gagtgcaaat gttgcaatat ctgcggcacc tccgagaatg acgaccagtt |
| 1201 | gctcttctgt gatgactgcg atcgtggcta ccacatgtac tgtctcaccc cgtccatgtc |
| 1261 | tgagccccct gaaggaagtt ggagctgcca cctgtgtctg gacctgttga aagagaaagc |
| 1321 | ttccatctac cagaaccaga actcctcttg atgtggccac ccacctgctc cccgacatat |
| 1381 | ctaaggctgt ttctctcctc cacttcatat ttcataccca tctttccctt cttcctcctc |
| 1441 | tccttcacaa atccagagaa ccttggggtg gttgtgccag cctgcctttg gcagctgcaa |
| 1501 | gctgaggtgg cagctctgac cacctctggc cccaggccct cagggagaaa ggagcaacac |
| 1561 | actgccccta ggcgtgcgtg tggcccagtt tctctctgct ctccattaag tgcattcact |
| 1621 | ctgcttgcct tgggcccagc ccctggtgat cacagggttc aaacagtgtc ctcctagaaa |
| 1681 | gagtgggaga gcagctcact tctctgtgtt ctgcctcccc tctggtctcc agagttttcc |
| 1741 | tgtcctctag aggcaagcca ggccagggag ctgggagcga gcaagctgag gccacgtcca |
| 1801 | caaggagctt ttcatgcccc tgtgccgcat agcctcacct ctttcctcca gagtggctct |
| 1861 | ctgcggccct gtgttcctgc tacagagtgt tcttttctgg agtcaggatg ttctcggtca |
| 1921 | ccctcctggt tctgccctgt cccattccac cccaccccag ggggaacagt agcttcacct |
| 1981 | tgttattccc attgctctcc tggctcactc ttacggtcgg tctccagtga ctgaagcatt |
| 2041 | ccccaccctt ggaatttctc atcttctgcc tcccttccta ctccttttgg ttttgtgggg |
| 2101 | agaggggaag gatcaggggg ccaggccagc agctcggggg ccacaaggag atggataatg |
| 2161 | tgcctgtttt ttaacacaac aaaaaagcct acctccaaaa tccccttttt gttcttcctg |
| 2221 | gacctgggca ttcagcctcc tgctcttaac tgaattggga gcctctgcca cctgccccgt |
| 2281 | gtatcctggc tctcagctca tggggaagcc acatagacat ccctttcttc ccttgcacgc |
| 2341 | tcgctagcag ctggtaaggt cttcacaccc tgattcctca agttttctgc ttagtggcac |
| 2401 | tgacattaag tagtgggggg acagtccatg ccaggacacc ctggagtagc cttccccctt |
| 2461 | ggccgtgggc aggccctaac tcactgtcgc tttggagttg aggtgtcttt tttttttctt |
| 2521 | tctttagttc ctgtattcta aacattagta aaaataaatg tttttacaca gagccctctg |
| 2581 | ctggatggtt tatctcctgc ctttctccat taagaaggcc atttcatcct aagatttcca |
| 2641 | tgatggtggt tttttttttt aatgttttga aatacagctt ttttcccccc aaattaaaat |
| 2701 | ttttttgtgg aaccccaata tgtaaagcga atataaaatt ggttattttg ttttgttaca |
| 2761 | taaattcaag tttataacaa ttctttgtta taaagaacaa tgaagctgtt ttgatcaata |
| 2821 | caaaatttgg gttaaaatca actttaacat ctatttttat gtttcagttg atttggagaa |
| 2881 | ttctcctagt cttggataca tagatggaag tgatgacagg tttataacag ttgaccttgc |
| 2941 | aatctcagac atttaaaaca ggaccagaag tttatataaa tataattaat aagcaaacta |
| 3001 | atgacatcac catgggacac acacaaaagt tcttgcagga gcagggtctg tgtggcttca |
| 3061 | gttgcctgca gcgctcccag gccagagcaa gtgctctagg atctgaactg cccgcagtgc |
| 3121 | agccctgcag cctttcccag ggcacgttga tgtgcacaca gtttccctga aggcaaagtg |
| 3181 | aacatgtgga gagcttacgt ggcagcgcgt atgtcttcag tgtgtgtttt agaagtccaa |
| 3241 | ctgttgtttt tatgttttta aaggaaagat ttgaatcaag cagttatggg ccccctgaag |
| 3301 | tatccttttt tctagaacat tctgaaagtc atccttgcct atgggaagcc taggccggcc |
| 3361 | tgcactgtta tgttcaataa ataagcaggg tgctctgggc tggggattgt gtgaggagca |
| 3421 | gagcgcagcc cgtcctcatg cttttccact gaagtaggcc aggcagagag ggagtacagc |
| 3481 | aatggatgcg ctttggcagc tgagtagtcc gagagccaga aaagaaatgt ggaaaataag |
| 3541 | aacgctgtag caggcctagg tgaggaaatt taggaagggt ttgcgggagg taggatttga |
| 3601 | gatgggtctt ggagagttgg acagtgtcag ccggtaggac gggggtgcgg acggaagcct |
| 3661 | gtgaggaagg cagaggatgc ggagctgtga gcggagggag cagcgaggct ggagagcagc |
| 3721 | tgggctgcgg gtcaagacgt ctgcgtttaa ttcgggactg aaggttagca gggaagggaa |
| 3781 | cgatgccaga tcttgagttt aagaacttga atcttgtaaa gtaccaaatc taataaaata |
| 3841 | ctcgtcctaa ataaaaaaaa aaaaaaaaaa aaaaaaaaaa aaaaaaaaaa aaaaaaa |

SEQ ID NO: 120 Human DPF2 Amino Acid Sequence Isoform 2 (NP_001317237.1)

| | |
|---|---|
| 1 | maavvenvvk llgeqyykda meqchnynar lcaersvrlp fldsqtgvaq sncyiwmekr |
| 61 | hrgpglasgq lysyparrwr kkrrahpped prlsfpsikp dtdqtlkkeg lisqdgssle |
| 121 | allrtdplek rgapdprvdd dslgefpvtn srarkrilep ddflddldde dyeedtpkrr |
| 181 | gkgkskgkgv gsarkkldas iledrdkpya cdnsfkqkht skapqrvcgk ryknrpglsy |
| 241 | hyahshlaee egedkedsqp ptpvsqrsee qkskkgpdgl alpnnycdfc lgdskinkkt |
| 301 | gqpeelvscs dcgrsghpsc lqftpvmmaa vktyrwqcie ckccnicgts enddqllfcd |
| 361 | dcdrgyhmyc ltpsmseppe gswschlcld llkekasiyq nqnss |

SEQ ID NO: 121 Mouse DPF2 cDNA Sequence Variant 1 (NM_001291078.1, CDS:
100-1317)

| | |
|---|---|
| 1 | cctgcgcaga gggtcgagga ccctgtgtcc tgagaaggct tagcgcctgc gcgttgtagg |
| 61 | tttcggggcc tcccggcctg agggagagga acagggaaga tggcggctgt ggtggagaat |
| 121 | gtagtgaagc tccttggcga gcaatactac aaagatgcca tggaacagtg ccacaattat |
| 181 | aacgcccgcc tctgtgctga acgtagtgtg cgcctgcctt tcctggactc acagactgga |
| 241 | gtagcccaga gcaattgtta tatctggatg gaaaagcgac accggggacc aggattggcc |
| 301 | tctggacagt tatactccta tcctgccaga cgctggcgga aaaagcgccg agcccaccca |
| 361 | cctgaggatc ccaggctttc tttcccatcg attaaaccag acactgacca gactctgaag |
| 421 | aaagaggggc ttatctctca ggatggcagc agtttagagg ctctgttgcg tactgatccc |
| 481 | ctggagaaac ggggtgcccc agatccccga gttgacgatg acagcctggg cgagtttcct |
| 541 | gttagcaaca gtcgagcacg gaagcggatc attgaacccg atgacttcct tgatgacctt |
| 601 | gatgatgagg actatgaaga agatacgcca aagcgtcggg ggaaggggaa gtccaagagt |
| 661 | aagggtgtga gcagtgcccg gaagaagctg gatgcttcca tcctggagga ccgggataag |
| 721 | ccctatgcct gtgacaatag tttcaaacaa aagcatacct cgaaagcgcc ccagagagtt |
| 781 | tgtggaaaac gttacaagaa ccgacctggc ctcagttacc actatgccca ctcccacctg |
| 841 | gctgaagagg aaggagagga caaagaagac tcccgacccc ccactcctgt gtcccagagg |
| 901 | tctgaggagc agaaatccaa gaaaggacct gatggattgg ccctgcctaa caactactgt |
| 961 | gacttctgcc taggagactc aaaaatcaac aagaagacag ggcagcccga ggagctagtg |
| 1021 | tcctgttccg actgtggccg ctcagggcat ccgtcctgcc tgcagttcac ccctgtgatg |
| 1081 | atggcggccg tgaagaccta ccgctggcag tgcatcgaat gcaagtgctg caacctctgc |
| 1141 | ggcacgtcgg agaacgatga ccagctactt ttctgtgatg actgtgaccg tggctaccac |
| 1201 | atgtactgtc tcactccttc catgtctgag cctcctgaag gaagttggag ttgccacctg |
| 1261 | tgtctggatc tgctgaagga gaaagcatcc atctaccaga accagaactc ctcctgatgt |
| 1321 | gccacccagc tcccctgcat ctaaggccgt tgctctcctc tctaccttgg tttccattgc |
| 1381 | ccctctctcc tctttcactc tgtagtcctg ccaacctccg ttggcaacag cacagggagg |
| 1441 | tggcagctct gactgcctct agccccgagc cctcagggag taaggagcag cgtgctgctc |
| 1501 | cagggctgac ctgtgggtcc aacttctctc tgctctccaa gaagtgcatt cactctgcct |
| 1561 | gccttgggcc taagaccctg gtgattacag ggctcaaatg gggtcctctg agaaggaata |
| 1621 | tgagagcagc tcacttgtct caagccttgc ccacccctct tcccccaaac cccctttggt |
| 1681 | ttccagggtt ttgccccaga gatgagccag gctgggcctt tcctggaagc agctggagtg |
| 1741 | agctggctga gtggcacttg ccaggacctt ttcataccct agttctgctt ccctttgcct |
| 1801 | cctgccaaag cagtcccctg tcctctgtca tgctacatgg ggttctgtgc ttgagctaga |
| 1861 | atgttctcgg gcacctcctg gctctgccct gtcccacaaa gggacgagca gcttcaaacc |
| 1921 | tgtcctccct gtgcttggtg gcttgctcac aggtgcgctc tggctaccca gacatttcct |
| 1981 | atcctcagaa cttcccatct tctgccccca tccttagtcc ctttgctttt gtagggagag |
| 2041 | ggatagtgtc aggggctggg ccagcagctt gggggccaca gggagaagtt ggataatgtg |
| 2101 | cctgtttttt aactcgataa aaaagcctac ctccaaaatt ccctttttgt tcttcctgaa |
| 2161 | cctgggcatt cagcctcctg tccttaacta aattaggagc ctctgcctcc tgcctgtgta |
| 2221 | tcctggctcc caggacacag gatggtcccc tttccttgca cgctagctag tagctggtaa |
| 2281 | ggtcttcaca ccctgagttt tctgtttcct gcttagtggc actgacatta agtaggaggg |
| 2341 | gacagtcctc tgcagtactc tagagagtgg gcttccccct tggctgtggg caggccctaa |
| 2401 | ctgttttctg caaagttgag ggccccccct cgcatattta gttcctgtat tcaaaacatt |
| 2461 | agtaaaaata aacattttta cagagtcttc tgctggacag tttgtctctt gactccttgt |
| 2521 | tgaaaggttg tttcatttca aacttacgac aatagggttt tttgttggtg gtggttggtt |
| 2581 | gttttaaatt gaaacaactt tttctcccaa aatcaaagtt tttgttaaac tccaccatgt |
| 2641 | aaaattattt tgttagtttt gttatgtaaa ttcagattta taacaattta gtggtataaa |
| 2701 | ggatgaagct aattaataca aaaattgggt taaaatcaac tttagcattt tctctgtatc |
| 2761 | tgtgcttttg gctggttgga aagactttac tcggtgtgaa tatgtaggcg gaggtgcggc |
| 2821 | agatctatgg cactgcagtg tctcctggtt aaagtgaacc cagaagcttg tttgtgcttt |
| 2881 | aaactccaag gagttatgag ttaagcctgg agagagagcg cagcagagga gaggatgctc |
| 2941 | gttgttcttg cagagggcca agtttggttc ccagcactca aatccggtgg ctcacaacca |
| 3001 | cctgtagctc cagctccagg agctggggag gtcaactgtg ctcctgcaaa cacccacctg |
| 3061 | cccactcatc ttcatccatc tacaaaccta ccagtgtcat cgtagaacaa aagaagccga |
| 3121 | gaggagagta acctcagatc ctgtcatctg atgaaccttt tcattgcctg tcggattgct |
| 3181 | aagccaaagc agagttgcaa agccagaatt gtccacagtg cagggtgtca tgtgcagacc |
| 3241 | gtgagtgagt ttatatccag ccagattagt acttggatgt tatatagtgg atcttgtata |
| 3301 | gctcacttgg tatgtattaa cattttaact tttttctttt aaagatttat ttattt |

SEQ ID NO: 122 Mouse DPF2 Amino Acid Sequence Isoform 1 (NP_001278007.1)

| | |
|---|---|
| 1 | maavvenvvk llgeqyykda meqchnynar lcaersvrlp fldsqtgvaq sncyiwmekr |
| 61 | hrgpglasgq lysyparrwr kkrrahpped prlsfpsikp dtdqtlkkeg lisqdgssle |
| 121 | allrtdplek rgapdprvdd dslgefpvsn srarkriiep ddflddldde dyeedtpkrr |
| 181 | gkgkskskgv ssarkkldas iledrdkpya cdnsfkqkht skapqrvcgk ryknrpglsy |
| 241 | hyahshlaee egedkedsrp ptpvsqrsee qkskkgpdgl alpnnycdfc lgdskinkkt |
| 301 | gqpeelvscs dcgrsghpsc lqftpvmmaa vktyrwqcie ckccnlcgts enddqllfcd |
| 361 | dcdrgyhmyc ltpsmseppe gswschlcld llkekasiyq nqnss |

SEQ ID NO: 123 Mouse DPF2 cDNA Sequence Variant 2 (NM_011262.5, CDS: 100-
1275)

| | |
|---|---|
| 1 | cctgcgcaga gggtcgagga ccctgtgtcc tgagaaggct tagcgcctgc gcgttgtagg |
| 61 | tttcggggcc tcccggcctg agggagagga acagggaaga tggcggctgt ggtggagaat |
| 121 | gtagtgaagc tccttggcga gcaatactac aaagatgcca tggaacagtg ccacaattat |
| 181 | aacgcccgcc tctgtgctga acgtagtgtg cgcctgcctt tcctggactc acagactgga |
| 241 | gtagcccaga gcaattgtta tatctggatg gaaaagcgac accggggacc aggattggcc |
| 301 | tctggacagt tatactccta tcctgccaga cgctggcgga aaaagcgccg agcccaccca |
| 361 | cctgaggatc ccaggctttc tttcccatcg attaaaccag acactgacca gactctgaag |
| 421 | aaagaggggc ttatctctca ggatggcagc agtttagagg ctctgttgcg tactgatccc |
| 481 | ctggagaaac ggggtgcccc agatccccga gttgacgatg acagcctggg cgagtttcct |
| 541 | gttagcaaca gtcgagcacg gaagcggatc attgaacccg atgacttcct tgatgacctt |
| 601 | gatgatgagg actatgaaga agatacgcca aagcgtcggg ggaaggggaa gtccaagagt |
| 661 | aagggtgtga gcagtgcccg gaagaagctg gatgcttcca tcctggagga ccgggataag |
| 721 | ccctatgcct gtgacatttg tggaaaacgt tacaagaacc gacctggcct cagttaccac |
| 781 | tatgcccact cccacctggc tgaagaggaa ggagaggaca aagaagactc ccgacccccc |
| 841 | actcctgtgt cccagaggtc tgaggagcag aaatccaaga aaggacctga tggattggcc |
| 901 | ctgcctaaca actactgtga cttctgccta ggagactcaa aaatcaacaa gaagacaggg |
| 961 | cagcccgagg agctagtgtc ctgttccgac tgtggccgct cagggcatcc gtcctgcctg |
| 1021 | cagttcaccc ctgtgatgat ggcggccgtg aagacctacc gctggcagtg catcgaatgc |
| 1081 | aagtgctgca acctctgcgg cacgtcggag aacgatgacc agctactttt ctgtgatgac |
| 1141 | tgtgaccgtg gctaccacat gtactgtctc actccttcca tgtctgagcc tcctgaagga |
| 1201 | agttggagtt gccacctgtg tctggatctg ctgaaggaga aagcatccat ctaccagaac |
| 1261 | cagaactcct cctgatgtgc cacccagctc ccctgcatct aaggccgttg ctctcctctc |
| 1321 | taccttggtt tccattgccc ctctctcctc tttcactctg tagtcctgcc aacctccgtt |
| 1381 | ggcaacagca cagggaggtg gcagctctga ctgcctctag ccccgagccc tcagggagta |
| 1441 | aggagcagcg tgctgctcca gggctgacct gtgggtccaa cttctctctg ctctccaaga |
| 1501 | agtgcattca ctctgcctgc cttgggccta agaccctggt gattacaggg ctcaaatggg |
| 1561 | gtcctctgag aaggaatatg agagcagctc acttgtctca agccttgccc acccctcttc |
| 1621 | ccccaaaccc cctttggttt ccagggtttt gccccagaga tgagccaggc tgggcctttc |
| 1681 | ctggaagcag ctggagtgag ctggctgagt ggcacttgcc aggacctttt cataccctag |
| 1741 | ttctgcttcc ctttgcctcc tgccaaagca gtcccctgtc ctctgtcatg ctacatgggg |
| 1801 | ttctgtgctt gagctagaat gttctcgggc acctcctggc tctgccctgt cccacaaagg |
| 1861 | gacgagcagc ttcaaacctg tcctccctgt gcttggtggc ttgctcacag gtgcgctctg |
| 1921 | gctacccaga catttcctat cctcagaact tcccatcttc tgcccccatc cttagtccct |
| 1981 | ttgcttttgt agggagaggg atagtgtcag gggctgggcc agcagcttgg gggccacagg |
| 2041 | gagaagttgg ataatgtgcc tgttttttaa ctcgataaaa aagcctacct ccaaaattcc |
| 2101 | ctttttgttc ttcctgaacc tgggcattca gcctcctgtc cttaactaaa ttaggagcct |
| 2161 | ctgcctcctg cctgtgtatc ctggctccca ggacacagga tggtcccctt tccttgcacg |
| 2221 | ctagctagta gctggtaagg tcttcacacc ctgagttttc tgtttcctgc ttagtggcac |
| 2281 | tgacattaag taggagggga cagtcctctg cagtactcta gagagtgggc ttcccccttg |
| 2341 | gctgtgggca ggccctaact gttttctgca aagttgaggg ccccccctcg catatttagt |
| 2401 | tcctgtattc aaaacattag taaaaataaa catttttaca gagtcttctg ctggacagtt |
| 2461 | tgtctcttga ctccttgttg aaaggttgtt tcatttcaaa cttacgacaa tagggttttt |
| 2521 | tgttggtggt ggttggttgt tttaaattga aacaactttt tctcccaaaa tcaaagtttt |
| 2581 | tgttaaactc caccatgtaa aattattttg ttagttttgt tatgtaaatt cagatttata |
| 2641 | acaatttagt ggtataaagg atgaagctaa ttaatacaaa aattgggtta aaatcaactt |
| 2701 | tagcattttc tctgtatctg tgcttttggc tggttggaaa gactttactc ggtgtgaata |
| 2761 | tgtaggcgga ggtgcggcag atctatggca ctgcagtgtc tcctggttaa agtgaaccca |
| 2821 | gaagcttgtt tgtgctttaa actccaagga gttatgagtt aagcctggag agagagcgca |
| 2881 | gcagaggaga ggatgctcgt tgttcttgca gagggccaag tttggttccc agcactcaaa |
| 2941 | tccggtggct cacaaccacc tgtagctcca gctccaggag ctggggaggt caactgtgct |
| 3001 | cctgcaaaca cccacctgcc cactcatctt catccatcta caaacctacc agtgtcatcg |
| 3061 | tagaacaaaa gaagccgaga ggagagtaac ctcagatcct gtcatctgat gaaccttttc |
| 3121 | attgcctgtc ggattgctaa gccaaagcag agttgcaaag ccagaattgt ccacagtgca |
| 3181 | gggtgtcatg tgcagaccgt gagtgagttt atatccagcc agattagtac ttggatgtta |
| 3241 | tatagtggat cttgtatagc tcacttggta tgtattaaca ttttaacttt tttcttttaa |
| 3301 | agatttattt attt |

SEQ ID NO: 124 Mouse DPF2 Amino Acid Sequence Isoform 2 (NP_035392.1)

| | |
|---|---|
| 1 | maavvenvvk llgeqyykda meqchnynar lcaersvrlp fldsqtgvaq sncyiwmekr |
| 61 | hrgpglasgq lysyparrwr kkrrahpped prlsfpsikp dtdqtlkkeg lisqdgssle |
| 121 | allrtdplek rgapdprvdd dslgefpvsn srarkriiep ddflddldde dyeedtpkrr |
| 181 | gkgkskskgv ssarkkldas iledrdkpya cdicgkrykn rpglsyhyah shlaeeeged |
| 241 | kedsrpptpv sqrseeqksk kgpdglalpn nycdfclgds kinkktgqpe elvscsdcgr |
| 301 | sghpsclqft pvmmaavkty rwqcieckcc nlcgtsendd qllfcddcdr gyhmycltps |
| 361 | mseppegsws chlcldllke kasiyqnqns s |

SEQ ID NO: 125 Human DPF3 cDNA Sequence Variant 1 (NM_012074.4, CDS: 29-
1102)

| | |
|---|---|
| 1 | agacaatatt ctgttacatt gtagcaaaat ggcgactgtc attcacaacc ccctgaaagc |
| 61 | gctcggggac cagttctaca aggaagccat tgagcactgc cggagttaca actcacggct |
| 121 | gtgtgcagag cgcagcgtgc gtcttccctt cctggactca cagactgggg tggcccagaa |
| 181 | caactgctac atctggatgg agaagaggca ccgaggccca ggccttgccc cgggccagct |
| 241 | gtatacatac cctgcccgct gctggcgcaa gaagagacga ttgcacccac ctgaagatcc |
| 301 | aaaactgcgg ctgctggaga taaaacctga agtggagctt cccctgaaga aggatgggtt |
| 361 | cacctcagag agcaccacgc tggaagcctt gctccgtggc gagggggttg agaagaaggt |
| 421 | ggatgccagg gaggaggaaa gcatccagga aatacagagg gttttggaaa atgatgaaaa |
| 481 | tgtagaagaa gggaatgaag aagaggattt ggaagaggat attcccaagc gaaagaacag |
| 541 | gactagagga cgggctcgcg gctctgcagg gggcaggagg aggcacgacg ccgcctctca |
| 601 | ggaagaccac gacaaacctt acgtctgtga catctgtggc aagcgctaca agaaccgacc |
| 661 | ggggctcagc taccactatg ctcacactca cctggccagc gaggaggggg atgaagctca |
| 721 | agaccaggag actcggtccc cacccaacca cagaaatgag aaccacaggc cccagaaagg |
| 781 | accggatgga acagtcattc ccaataacta ctgtgacttc tgcttggggg gctccaacat |
| 841 | gaacaagaag agtgggcggc ctgaagagct ggtgtcctgc gcagactgtg gacgctctgc |
| 901 | tcatttggga ggagaaggca ggaaggagaa ggaggcagcg gccgcagcac gtaccacgga |
| 961 | ggacttattc ggttccacgt cagaaagtga cacgtcaact ttccacggct ttgatgagga |
| 1021 | cgatttggaa gagcctcgct cctgtcgagg acgccgcagt ggccggggtt cgcccacagc |
| 1081 | agataaaaag ggcagttgct aaacccacgg aacagactct ctgggcaatt agccatcccc |
| 1141 | ctctgacttt ggtcattgtg ctggttctga tatatatttt ttttaatgaa aggcaacttt |
| 1201 | agattttccc tctatccttg ctttttttcc cttcacctcc cacgtgtccc tccatccctc |
| 1261 | cccccacccc tctgttttgg gtatgtacaa cagaagcaca aactactgaa acaaaacaaa |
| 1321 | acagcagaat gagcgttctt ccgagagatg gcatcgtgat gcgctattta ttttccatag |
| 1381 | aaataggaag ttagacggat tgtctctttt ctgaggggag ggggtctttt tgacaggagc |
| 1441 | agagttgatg tcctcaattt tcatatttat tggcaaaagg aagagaagag gaactttggg |
| 1501 | ttggaaacaa agaaccaata acattaaaac attattattt atatattcta gctgttatta |
| 1561 | gaatcagact ttttttgcga gagagagaga gagagagaga gaagggaaat caaagaaatc |
| 1621 | gaagcaatat cctgtttaga ggcaagccgc ccggtgggga gaatttcctc aatgggagac |
| 1681 | ggttgcacta ttctgtgccc cacggagttt gcggctcccc gcggcagacc cctccctcat |
| 1741 | tctcctccct gacctttcca tcttcctctc tgcttgcgag aaaatgtcag tagttccaga |
| 1801 | gaagtcgggg tgcctatgcc tggcctccct ccacacctgg gccctgacca gccgcctcct |
| 1861 | gggctcctcc tcctccgtca gtagagctgc tgttttgtta ttgctggttt ttcctcactt |
| 1921 | tcctcctggc aaagaacgac ttccaaatgc agggatggaa tataagcaga acgtcatggg |
| 1981 | ctcagcagtg actccaccac ccgaggccga ggccgtgctt ctggaagata gaaggagaca |
| 2041 | tcatcgtgtg tttcccctcc ccttgcccct gttaagaaac gtatcaatac ccattggatg |
| 2101 | atcaaggcta ccgtatttct tctatttttt tttatagtgc ctgccaggca ctttgtttta |
| 2161 | tgtttccaat agcacttcct gaaataaacc aaagcaacac tgctcaaggc ccctggggcg |
| 2221 | atggagaagg ccacccacct cactgacagt cccaagaatg accggctgcg aggtcctagt |
| 2281 | caaaagtcaa cattatgacc tggggactcc agcatccttc aagcaagcca tttccgaaga |
| 2341 | aggtgaaaag aagccaggat gattggcacc tcctcctcct cctcctcttc ttcctcttcc |
| 2401 | cttgcccagc cccctcctgt gcgtgtgttt cagacaacac aggagccagc acaggagtgg |
| 2461 | aaaatcctgc agcgcaactc agctcagccc acagaagcct tgggaatggc ctcagtttgt |
| 2521 | gcaataagaa gatttttttt ttctttttaa atcttcatta tattttcttt gattgtctgt |
| 2581 | gagaaagtac ccaggtccgc ctggaattac tctacagtag aaataactga acacaaacaa |
| 2641 | actgatggaa aaaaagagtt aactatttta tttatttcaa tatttaaaag gaaaaaagtg |
| 2701 | ctgacatggc acagtatttt tgtttaaagt acctcctact tcaaaagtta agcgcaattt |
| 2761 | tgtgaagaca tgaaatcata agagtactta atgtaaaata aaagactgca tattaactct |
| 2821 | aaagaaaaat gccccacatt ttaaataaga aaataaagat caactctgct ctctcaggct |
| 2881 | ttttaaaaag ccattcatgt atgtgcttta ggtattttta tttctgcgag ttggatgtgg |
| 2941 | taagtgagga gtgctcagtt tttttttcct ccttcaaaag tctattgaaa gtgttggtga |
| 3001 | tgttaaatga ttgtgtgtta agatttgact gaaataactt agccacaaat cagcagtttc |
| 3061 | ccccaccctc attgccccct caccccaggc aagccccttt tatctgaatg tcagaagcag |
| 3121 | cctgcctcct agttatcatg tctgatgagg tctagctcag gaaggaattc catctattga |
| 3181 | tggaatatat cccctcaagt tcaatagatt cgaacacaga gagctttgtt taaaataatg |
| 3241 | cagcaaaaaa aaaaaaaaaa aaaaagcaaa aataaaagca tcagctgagg tgatattagt |
| 3301 | tcagtcacct aacaactcct agaagagatg aggaaaggga accttctgct gagctggctt |
| 3361 | ctggggcctg agcttccaga gctgtcccca agggctagga aggccgacct gaaggatgag |
| 3421 | aacctcaaat tcagttgctg gtgggagcca aggaagacgg cgggtgttct aacatggccc |
| 3481 | tttctggctg agctggcgga agtgggcgtt ttggccgatg ggatgtatct cggcgctgtg |
| 3541 | tctgtggccc agcaaaggtg cagggctgac tggctgagcc actgggttct acccgcaggc |
| 3601 | tccccactgc actgggcttt cacacagcca tgctcttggg tttccctccc ttgtaagcag |
| 3661 | agtcataata acacacgaat agtctaaggc tgggtattct ggtcagcaga ggtccttgag |
| 3721 | tcacagtgtt actgaaatgg ttctgagcct gagaatctct ttggcctctg aaagggcagg |
| 3781 | gcaggtgggc accgacttcc tgccagtcct ttcaggtttc ctgttcaaag ccagtcctgt |
| 3841 | tggtggaggg gatcaccgag agtgtctgta tcattttgta gcccttttct ctgacgtttt |
| 3901 | ctggtagaaa atgtcccttg tcaaaatgct aataattatc ataataatct gctttccaac |
| 3961 | caactcccac aagtgacaac ctgtgtagaa ctgtgataaa ggtttgcata atgtagggtt |
| 4021 | tgtaccaagt gtgtgtaagt ttctgttaaa taaaaagtct gtttccaatg ctcctat |

SEQ ID NO: 126 Human DPF3 Amino Acid Sequence Isoform 1 (NP_036206.3)

| | |
|---|---|
| 1 | matvihnplk algdqfykea iehcrsynsr lcaersvrlp fldsqtgvaq nncyiwmekr |
| 61 | hrgpglapgq lytyparcwr kkrrlhpped pklrlleikp evelplkkdg ftsesttlea |
| 121 | llrgegvekk vdareeesiq eiqrvlende nveegneeed leedipkrkn rtrgrargsa |
| 181 | ggrrrhdaas qedhdkpyvc dicgkryknr pglsyhyaht hlaseegdea qdqetrsppn |
| 241 | hrnenhrpqk gpdgtvipnn ycdfclggsn mnkksgrpee lvscadcgrs ahlggegrke |
| 301 | keaaaaartt edlfgstses dtstfhgfde ddleeprscr grrsgrgspt adkkgsc |

SEQ ID NO: 127 Human DPF3 cDNA Sequence Variant 2 (NM_001280542.1, CDS:
29-1165)

| | |
|---|---|
| 1 | agacaatatt ctgttacatt gtagcaaaat ggcgactgtc attcacaacc ccctgaaagc |
| 61 | gctcggggac cagttctaca aggaagccat tgagcactgc cggagttaca actcacggct |
| 121 | gtgtgcagag cgcagcgtgc gtcttccctt cctggactca cagactgggg tggcccagaa |
| 181 | caactgctac atctggatgg agaagaggca ccgaggccca ggccttgccc cgggccagct |
| 241 | gtatacatac cctgcccgct gctggcgcaa gaagagacga ttgcacccac ctgaagatcc |
| 301 | aaaactgcgg ctgctggaga taaaacctga agtggagctt cccctgaaga aggatgggtt |
| 361 | cacctcagag agcaccacgc tggaagcctt gctccgtggc gagggggttg agaagaaggt |
| 421 | ggatgccagg gaggaggaaa gcatccagga aatacagagg gttttggaaa atgatgaaaa |
| 481 | tgtagaagaa gggaatgaag aagaggattt ggaagaggat attcccaagc gaaagaacag |
| 541 | gactagagga cgggctcgcg gctctgcagg gggcaggagg aggcacgacg ccgcctctca |
| 601 | ggaagaccac gacaaacctt acgtctgtga catctgtggc aagcgctaca agaaccgacc |
| 661 | ggggctcagc taccactatg ctcacactca cctggccagc gaggaggggg atgaagctca |
| 721 | agaccaggag actcggtccc cacccaacca cagaaatgag aaccacaggc cccagaaagg |
| 781 | accggatgga acagtcattc ccaataacta ctgtgacttc tgcttggggg gctccaacat |
| 841 | gaacaagaag agtgggcggc ctgaagagct ggtgtcctgc gcagactgtg gacgctctgg |
| 901 | tcacccaacc tgcctgcagt ttaccctgaa catgaccgag gctgtcaaga cctacaagtg |
| 961 | gcagtgcata gagtgcaaat cctgtatcct ctgtgggacc tcagagaatg atgaccagct |
| 1021 | actcttctgc gatgactgtg accgaggcta tcacatgtac tgtttaaatc ccccggtggc |
| 1081 | tgagccccca gaaggaagct ggagctgcca cttatgctgg gaactgctca aagagaaagc |
| 1141 | ctcagccttt ggctgccagg cctagg |

SEQ ID NO: 128 Human DPF3 Amino Acid Sequence Isoform 2 (NP_001267471.1)

| | |
|---|---|
| 1 | matvihnplk algdqfykea iehcrsynsr lcaersvrlp fldsqtgvaq nncyiwmekr |
| 61 | hrgpglapgq lytyparcwr kkrrlhpped pklrlleikp evelplkkdg ftsesttlea |
| 121 | llrgegvekk vdareeesiq eiqrvlende nveegneeed leedipkrkn rtrgrargsa |
| 181 | ggrrrhdaas qedhdkpyvc dicgkryknr pglsyhyaht hlaseegdea qdqetrsppn |
| 241 | hrnenhrpqk gpdgtvipnn ycdfclggsn mnkksgrpee lvscadcgrs ghptclqftl |
| 301 | nmteavktyk wqciecksci lcgtsenddq llfcddcdrg yhmyclnppv aeppegswsc |
| 361 | hlcwellkek asafgcqa |

SEQ ID NO: 129 Human DPF3 cDNA Sequence Variant 3 (NM_001280543.1, CDS:
143-1246)

| | |
|---|---|
| 1 | agacaatatt ctgttacatt gtagcaaaat ggcgactgtc attcacaacc ccctgaaagc |
| 61 | gccctttcaa gaatcctatg aaagttgtgg atcatctccc cggaaaacac gcatatagat |
| 121 | gtgaacatct gcctatggtt ttatggggtt cacagacctg gaagagccca tctctggatg |
| 181 | ccctggaggc ccatgggctc tagggctcgg ggaccagttc tacaaggaag ccattgagca |
| 241 | ctgccggagt tacaactcac ggctgtgtgc agagcgcagc gtgcgtcttc ccttcctgga |
| 301 | ctcacagact ggggtggccc agaacaactg ctacatctgg atggagaaga ggcaccgagg |
| 361 | cccaggcctt gccccgggcc agctgtatac ataccctgcc cgctgctggc gcaagaagag |
| 421 | acgattgcac ccacctgaag atccaaaact gcggctgctg gagataaaac ctgaagtgga |
| 481 | gcttcccctg aagaaggatg ggttcacctc agagagcacc acgctggaag ccttgctccg |
| 541 | tggcgagggg gttgagaaga aggtggatgc cagggaggag gaaagcatcc aggaaataca |
| 601 | gagggttttg gaaaatgatg aaaatgtaga agaagggaat gaagaagagg atttggaaga |
| 661 | ggatattccc aagcgaaaga acaggactag aggacgggct cgcggctctg cagggggcag |
| 721 | gaggaggcac gacgccgcct ctcaggaaga ccacgacaaa ccttacgtct gtgacatctg |
| 781 | tggcaagcgc tacaagaacc gaccggggct cagctaccac tatgctcaca ctcacctggc |
| 841 | cagcgaggag ggggatgaag ctcaagacca ggagactcgg tccccaccca accacagaaa |
| 901 | tgagaaccac aggccccaga aaggaccgga tggaacagtc attcccaata actactgtga |
| 961 | cttctgcttg gggggctcca acatgaacaa gaagagtggg cggcctgaag agctggtgtc |
| 1021 | ctgcgcagac tgtggacgct ctgctcattt gggaggagaa ggcaggaagg agaaggaggc |
| 1081 | agcggccgca gcacgtacca cggaggactt attcggttcc acgtcagaaa gtgacacgtc |
| 1141 | aactttccac ggctttgatg aggacgattt ggaagagcct cgctcctgtc gaggacgccg |
| 1201 | cagtggccgg ggttcgccca cagcagataa aaagggcagt tgctaaaccc acggaacaga |
| 1261 | ctctctgggc aattagccat ccccctctga ctttggtcat tgtgctggtt ctgatatata |
| 1321 | ttttttttaa tgaaaggcaa ctttagattt tccctctatc cttgcttttt ttcccttcac |
| 1381 | ctcccacgtg tccctccatc cctcccccca cccctctgtt ttgggtatgt acaacagaag |
| 1441 | cacaaactac tgaaacaaaa caaaacagca gaatgagcgt tcttccgaga gatggcatcg |
| 1501 | tgatgcgcta tttattttcc atagaaatag gaagttagac ggattgtctc ttttctgagg |
| 1561 | ggagggggtc tttttgacag gagcagagtt gatgtcctca attttcatat ttattggcaa |
| 1621 | aaggaagaga agaggaactt tgggttggaa acaaagaacc aataacatta aaacattatt |
| 1681 | atttatatat tctagctgtt attagaatca gacttttttt gcgagagaga gagagagaga |
| 1741 | gagagaaggg aaatcaaaga aatcgaagca atatcctgtt tagaggcaag ccgcccggtg |
| 1801 | gggagaattt cctcaatggg agacggttgc actattctgt gccccacgga gtttgcggct |
| 1861 | ccccgcggca gacccctccc tcattctcct ccctgacctt tccatcttcc tctctgcttg |
| 1921 | cgagaaaatg tcagtagttc cagagaagtc ggggtgccta tgcctggcct ccctccacac |
| 1981 | ctgggccctg accagccgcc tcctgggctc ctcctcctcc gtcagtagag ctgctgtttt |
| 2041 | gttattgctg gtttttcctc actttcctcc tggcaaagaa cgacttccaa atgcagggat |
| 2101 | ggaatataag cagaacgtca tgggctcagc agtgactcca ccacccgagg ccgaggccgt |
| 2161 | gcttctggaa gatagaagga gacatcatcg tgtgtttccc ctccccttgc ccctgttaag |
| 2221 | aaacgtatca atacccattg gatgatcaag gctaccgtat ttcttctatt tttttttata |
| 2281 | gtgcctgcca ggcactttgt tttatgtttc caatagcact tcctgaaata aaccaaagca |
| 2341 | acactgctca aggcccctgg ggcgatggag aaggccaccc acctcactga cagtcccaag |
| 2401 | aatgaccggc tgcgaggtcc tagtcaaaag tcaacattat gacctgggga ctccagcatc |
| 2461 | cttcaagcaa gccatttccg aagaaggtga aaagaagcca ggatgattgg cacctcctcc |
| 2521 | tcctcctcct cttcttcctc ttcccttgcc cagccccctc ctgtgcgtgt gtttcagaca |
| 2581 | acacaggagc cagcacagga gtggaaaatc ctgcagcgca actcagctca gcccacagaa |
| 2641 | gccttgggaa tggcctcagt ttgtgcaata agaagatttt ttttttcttt ttaaatcttc |
| 2701 | attatatttt ctttgattgt ctgtgagaaa gtacccaggt ccgcctggaa ttactctaca |
| 2761 | gtagaaataa ctgaacacaa acaaactgat ggaaaaaaag agttaactat tttatttatt |
| 2821 | tcaatattta aaaggaaaaa agtgctgaca tggcacagta tttttgttta aagtacctcc |
| 2881 | tacttcaaaa gttaagcgca attttgtgaa gacatgaaat cataagagta cttaatgtaa |
| 2941 | aataaaagac tgcatattaa ctctaaagaa aaatgcccca cattttaaat aagaaaataa |
| 3001 | agatcaactc tgctctctca ggctttttaa aaagccattc atgtatgtgc tttaggtatt |
| 3061 | tttatttctg cgagttggat gtggtaagtg aggagtgctc agtttttttt tcctccttca |
| 3121 | aaagtctatt gaaagtgttg gtgatgttaa atgattgtgt gttaagattt gactgaaata |
| 3181 | acttagccac aaatcagcag tttcccccac cctcattgcc ccctcacccc aggcaagccc |
| 3241 | cttttatctg aatgtcagaa gcagcctgcc tcctagttat catgtctgat gaggtctagc |
| 3301 | tcaggaagga attccatcta ttgatggaat atatcccctc aagttcaata gattcgaaca |
| 3361 | cagagagctt tgtttaaaat aatgcagcaa aaaaaaaaaa aaaaaaaaag caaaaataaa |
| 3421 | agcatcagct gaggtgatat tagttcagtc acctaacaac tcctagaaga gatgaggaaa |
| 3481 | gggaaccttc tgctgagctg gcttctgggg cctgagcttc cagagctgtc cccaagggct |
| 3541 | aggaaggccg acctgaagga tgagaacctc aaattcagtt gctggtggga gccaaggaag |
| 3601 | acggcgggtg ttctaacatg gccctttctg gctgagctgg cggaagtggg cgttttggcc |
| 3661 | gatgggatgt atctcggcgc tgtgtctgtg gcccagcaaa ggtgcagggc tgactggctg |
| 3721 | agccactggg ttctacccgc aggctcccca ctgcactggg ctttcacaca gccatgctct |
| 3781 | tgggtttccc tcccttgtaa gcagagtcat aataacacac gaatagtcta aggctgggta |
| 3841 | ttctggtcag cagaggtcct tgagtcacag tgttactgaa atggttctga gcctgagaat |
| 3901 | ctctttggcc tctgaaaggg cagggcaggt gggcaccgac ttcctgccag tcctttcagg |
| 3961 | tttcctgttc aaagccagtc ctgttggtgg aggggatcac cgagagtgtc tgtatcattt |
| 4021 | tgtagccctt ttctctgacg ttttctggta gaaaatgtcc cttgtcaaaa tgctaataat |
| 4081 | tatcataata atctgctttc caaccaactc ccacaagtga caacctgtgt agaactgtga |
| 4141 | taaaggtttg cataatgtag ggtttgtacc aagtgtgtgt aagtttctgt taaataaaaa |
| 4201 | gtctgtttcc aatgctccta t |

SEQ ID NO: 130 Human DPF3 Amino Acid Sequence Isoform 3 (NP_001267472.1)

| | |
|---|---|
| 1 | mgftdleepi sgcpggpwal glgdqfykea iehcrsynsr lcaersvrlp fldsqtgvaq |
| 61 | nncyiwmekr hrgpglapgq lytyparcwr kkrrlhpped pklrlleikp evelplkkdg |
| 121 | ftsesttlea llrgegvekk vdareeesiq eiqrvlende nveegneeed leedipkrkn |
| 181 | rtrgrargsa ggrrrhdaas qedhdkpyvc dicgkryknr pglsyhyaht hlaseegdea |
| 241 | qdqetrsppn hrnenhrpqk gpdgtvipnn ycdfclggsn mnkksgrpee lvscadcgrs |
| 301 | ahlggegrke keaaaaartt edlfgstses dtstfhgfde ddleeprscr grrsgrgspt |
| 361 | adkkgsc |

SEQ ID NO: 131 Human DPF3 cDNA Sequence Variant 4 (NM_001280544.1, CDS:
307-1545)

| | |
|---|---|
| 1 | attctcgtct tcacccctgg ccactcctgg agttgaaaac caggttcgct cccggggacg |
| 61 | gtagggggtt cctaacgcaa aggaatgcac agggagaatc ggacgtgttt gcgccagctc |
| 121 | gtcgcccatc agaaataggg aaaggggtag gaaggcccca ggtttcaaat atatttatat |
| 181 | gaaagctgcc gttaagagga cgttggaagc tgaggctgat cagataggag ctcctggctt |
| 241 | cagttctggc tcggaagctc ggatacactg cgcttgaacg ccacagcgtt tcacccaaga |
| 301 | aagaaaatgt tttatggcag aataaatggg cgtaacttcg ccgcatcctc gctgccggtt |
| 361 | gctttcgctg caacaccgct gatgctgttt ctaccgaacc cacaactgat tttcagtttc |
| 421 | cccatttcca gccgaaatca cataaccggg ctgatgccac ctggtaaact caagttagag |
| 481 | aacctatttc acatgtgcac caggctcggg gaccagttct acaaggaagc cattgagcac |
| 541 | tgccggagtt acaactcacg gctgtgtgca gagcgcagcg tgcgtcttcc cttcctggac |
| 601 | tcacagactg gggtggccca gaacaactgc tacatctgga tggagaagag gcaccgaggc |
| 661 | ccaggccttg ccccgggcca gctgtataca taccctgccc gctgctggcg caagaagaga |
| 721 | cgattgcacc cacctgaaga tccaaaactg cggctgctgg agataaaacc tgaagtggag |
| 781 | cttcccctga agaaggatgg gttcacctca gagagcacca cgctggaagc cttgctccgt |
| 841 | ggcgaggggg ttgagaagaa ggtggatgcc agggaggagg aaagcatcca ggaaatacag |
| 901 | agggttttgg aaaatgatga aaatgtagaa gaagggaatg aagaagagga tttggaagag |
| 961 | gatattccca agcgaaagaa caggactaga ggacgggctc gcggctctgc agggggcagg |
| 1021 | aggaggcacg acgccgcctc tcaggaagac cacgacaaac cttacgtctg tgacatctgt |
| 1081 | ggcaagcgct acaagaaccg accggggctc agctaccact atgctcacac tcacctggcc |
| 1141 | agcgaggagg gggatgaagc tcaagaccag gagactcggt ccccacccaa ccacagaaat |
| 1201 | gagaaccaca ggccccagaa aggaccggat ggaacagtca ttcccaataa ctactgtgac |
| 1261 | ttctgcttgg ggggctccaa catgaacaag aagagtgggc ggcctgaaga gctggtgtcc |
| 1321 | tgcgcagact gtggacgctc tgctcatttg ggaggagaag gcaggaagga gaaggaggca |
| 1381 | gcggccgcag cacgtaccac ggaggactta ttcggttcca cgtcagaaag tgacacgtca |
| 1441 | actttccacg gctttgatga ggacgatttg gaagagcctc gctcctgtcg aggacgccgc |
| 1501 | agtggccggg gttcgcccac agcagataaa aagggcagtt gctaaaccca cggaacagac |
| 1561 | tctctgggca attagccatc cccctctgac tttggtcatt gtgctggttc tgatatatat |
| 1621 | tttttttaat gaaaggcaac tttagatttt ccctctatcc ttgctttttt tcccttcacc |
| 1681 | tcccacgtgt ccctccatcc ctccccccac ccctctgttt tgggtatgta caacagaagc |
| 1741 | acaaactact gaaacaaaac aaaacagcag aatgagcgtt cttccgagag atggcatcgt |
| 1801 | gatgcgctat ttattttcca tagaaatagg aagttagacg gattgtctct tttctgaggg |
| 1861 | gagggggtct ttttgacagg agcagagttg atgtcctcaa ttttcatatt tattggcaaa |
| 1921 | aggaagagaa gaggaacttt gggttggaaa caaagaacca ataacattaa aacattatta |
| 1981 | tttatatatt ctagctgtta ttagaatcag actttttttg cgagagagag agagagagag |
| 2041 | agagaaggga aatcaaagaa atcgaagcaa tatcctgttt agaggcaagc cgcccggtgg |
| 2101 | ggagaatttc ctcaatggga gacggttgca ctattctgtg ccccacggag tttgcggctc |
| 2161 | cccgcggcag acccctccct cattctcctc cctgaccttt ccatcttcct ctctgcttgc |
| 2221 | gagaaaatgt cagtagttcc agagaagtcg gggtgcctat gcctggcctc cctccacacc |
| 2281 | tgggccctga ccagccgcct cctgggctcc tcctcctccg tcagtagagc tgctgttttg |
| 2341 | ttattgctgg tttttcctca ctttcctcct ggcaaagaac gacttccaaa tgcagggatg |
| 2401 | gaatataagc agaacgtcat gggctcagca gtgactccac cacccgaggc cgaggccgtg |
| 2461 | cttctggaag atagaaggag acatcatcgt gtgtttcccc tccccttgcc cctgttaaga |
| 2521 | aacgtatcaa tacccattgg atgatcaagg ctaccgtatt tcttctattt ttttttatag |
| 2581 | tgcctgccag gcactttgtt ttatgtttcc aatagcactt cctgaaataa accaaagcaa |
| 2641 | cactgctcaa ggcccctggg gcgatggaga aggccaccca cctcactgac agtcccaaga |
| 2701 | atgaccggct gcgaggtcct agtcaaaagt caacattatg acctggggac tccagcatcc |
| 2761 | ttcaagcaag ccatttccga agaaggtgaa aagaagccag gatgattggc acctcctcct |
| 2821 | cctcctcctc ttcttcctct tcccttgccc agccccctcc tgtgcgtgtg tttcagacaa |
| 2881 | cacaggagcc agcacaggag tggaaaatcc tgcagcgcaa ctcagctcag cccacagaag |
| 2941 | ccttgggaat ggcctcagtt tgtgcaataa gaagattttt tttttctttt taaatcttca |
| 3001 | ttatattttc tttgattgtc tgtgagaaag tacccaggtc cgcctggaat tactctacag |
| 3061 | tagaaataac tgaacacaaa caaactgatg gaaaaaaaga gttaactatt ttatttattt |
| 3121 | caatatttaa aaggaaaaaa gtgctgacat ggcacagtat ttttgtttaa agtacctcct |
| 3181 | acttcaaaag ttaagcgcaa ttttgtgaag acatgaaatc ataagagtac ttaatgtaaa |
| 3241 | ataaaagact gcatattaac tctaaagaaa aatgccccac attttaaata agaaaataaa |
| 3301 | gatcaactct gctctctcag gctttttaaa aagccattca tgtatgtgct ttaggtattt |
| 3361 | ttatttctgc gagttggatg tggtaagtga ggagtgctca gttttttttt cctccttcaa |
| 3421 | aagtctattg aaagtgttgg tgatgttaaa tgattgtgtg ttaagatttg actgaaataa |
| 3481 | cttagccaca aatcagcagt ttcccccacc ctcattgccc cctcacccca ggcaagcccc |
| 3541 | ttttatctga atgtcagaag cagcctgcct cctagttatc atgtctgatg aggtctagct |
| 3601 | caggaaggaa ttccatctat tgatggaata tatcccctca agttcaatag attcgaacac |
| 3661 | agagagcttt gtttaaaata atgcagcaaa aaaaaaaaaa aaaaaaaagc aaaaataaaa |
| 3721 | gcatcagctg aggtgatatt agttcagtca cctaacaact cctagaagag atgaggaaag |
| 3781 | ggaaccttct gctgagctgg cttctggggc ctgagcttcc agagctgtcc ccaagggcta |
| 3841 | ggaaggccga cctgaaggat gagaacctca aattcagttg ctggtgggag ccaaggaaga |
| 3901 | cggcgggtgt tctaacatgg ccctttctgg ctgagctggc ggaagtgggc gttttggccg |
| 3961 | atgggatgta tctcggcgct gtgtctgtgg cccagcaaag gtgcagggct gactggctga |
| 4021 | gccactgggt tctacccgca ggctccccac tgcactgggc tttcacacag ccatgctctt |
| 4081 | gggtttccct cccttgtaag cagagtcata ataacacacg aatagtctaa ggctgggtat |
| 4141 | tctggtcagc agaggtcctt gagtcacagt gttactgaaa tggttctgag cctgagaatc |
| 4201 | tctttggcct ctgaaagggc agggcaggtg ggcaccgact tcctgccagt cctttcaggt |
| 4261 | ttcctgttca aagccagtcc tgttggtgga ggggatcacc gagagtgtct gtatcatttt |
| 4321 | gtagcccttt tctctgacgt tttctggtag aaaatgtccc ttgtcaaaat gctaataatt |
| 4381 | atcataataa tctgctttcc aaccaactcc cacaagtgac aacctgtgta gaactgtgat |
| 4441 | aaaggtttgc ataatgtagg gtttgtacca agtgtgtgta agtttctgtt aaataaaaag |
| 4501 | tctgtttcca atgctcctat |

SEQ ID NO: 132 Human DPF3 Amino Acid Sequence Isoform 4 (NP_001267473.1)

| | |
|---|---|
| 1 | mfygringrn faasslpvaf aatplmlflp npqlifsfpi ssrnhitglm ppgklklenl |
| 61 | fhmctrlgdq fykeaiehcr synsrlcaer svrlpfldsq tgvaqnncyi wmekrhrgpg |
| 121 | lapgqlytyp arcwrkkrrl hppedpklrl leikpevelp lkkdgftses ttleallrge |
| 181 | gvekkvdare eesiqeiqrv lendenveeg neeedleedi pkrknrtrgr argsaggrrr |
| 241 | hdaasqedhd kpyvcdicgk ryknrpglsy hyahthlase egdeaqdqet rsppnhrnen |
| 301 | hrpqkgpdgt vipnnycdfc lggsnmnkks grpeelvsca dcgrsahlgg egrkekeaaa |
| 361 | aarttedlfg stsesdtstf hgfdeddlee prscrgrrsg rgsptadkkg sc |

SEQ ID NO: 133 Mouse DPF3 cDNA Sequence Variant 1 (NM_001267625.1, CDS:
29-1165)

| | |
|---|---|
| 1 | agacaatatt ctgttacatt gtagcaaaat ggcgactgtc attcacaacc ccctgaaagc |
| 61 | gcttggggac cagttctaca aggaagccat tgagcactgc cggagctaca actcgaggct |
| 121 | gtgcgcagag cggagcgtgc gtctcccctt cctggactcg cagactgggg tggctcagaa |
| 181 | caactgctac atctggatgg agaagaggca ccgcggccca ggcctcgctc cgggccagtt |
| 241 | gtacacatac cctgcccgct gctggcgcaa gaagcgacga ttgcacccac cagaggaccc |
| 301 | aaaactacga ctcctggaaa tcaaacccga agtagaactg cccctgaaga aagatggatt |
| 361 | tacctctgag agtaccacac tggaagcctt gcttcgcggc gagggagtag agaagaaggt |
| 421 | ggatgccaga gaagaggaaa gcatccagga gatacagagg gttttggaaa atgatgaaaa |
| 481 | cgtagaagaa gggaatgaag aggaggattt ggaagaagat gttcccaagc gcaagaacag |
| 541 | gaccagagga cgggctcgcg gctctgcagg cggaaggagg aggcatgatg ccgcctctca |
| 601 | ggaagaccac gacaaaccct acgtctgcga catctgtggc aagcgctaca agaaccggcc |
| 661 | aggactcagc taccactacg ctcatactca cctggccagc gaggagggag acgaagccca |
| 721 | agaccaggag acccgatccc cacccaacca cagaaatgag aaccacagac cccagaaagg |
| 781 | accagacggg acagtcattc ctaataacta ctgtgacttc tgcttggggg gctccaacat |
| 841 | gaacaagaag agtgggaggc ctgaagagct ggtgtcctgt gcagactgtg gacgctctgg |
| 901 | tcatccaact tgcctgcagt tcactctgaa catgactgag gcagttaaga cctacaagtg |
| 961 | gcagtgcata gagtgtaaat cctgtatcct gtgtgggacc tcggagaacg acgaccagct |
| 1021 | actcttctgt gatgactgcg atcgtggcta tcacatgtac tgtttaaatc ccccagtggc |
| 1081 | tgagccccca gaaggaagct ggagctgcca tttatgctgg gagctgctca aagagaaagc |
| 1141 | atcagccttt ggctgccagg cctagggctc cacccaggtc acagagtgca gcccaccact |
| 1201 | agagaggctg aactgaagcc ctgttcaacc cagatggagg tctcctcctg tatatgcaca |
| 1261 | cagaccaact acaaggaaaa cgaatagtta cagaagggaa cggagggagc aaggtctcca |
| 1321 | ctcacttctc gccctaccca tgacctccca ccccacacat ccttcagcca gctcttcctc |
| 1381 | atttctacca gcgggaactt ggcacttttg aagaataatc cagccccggc tctgtggaaa |
| 1441 | cttcctcatg ttcactgtca caggcatctc tctttgttgc ttcttgtttt ggaggaagcc |
| 1501 | attttgtgac tgctcatcaa ccactcgtgt gttgcttggt ggggttcttg ttttgttgtc |
| 1561 | tattgtgttt caagaacttg tcacagagtg tcctcaccct tagcttaggc tcttcatcct |
| 1621 | gaaactcaca gaggaacaaa atgccgtggt ggggaagctc ctgcctatta cgagtctcac |
| 1681 | tggaagcatc catgtttgga ggccatcttg aagacagaac ttggaaaatg tcttggtttt |
| 1741 | cttagtctct gctgagaaga gaagttgtag catttgagcc ttggcagtag catccccagc |
| 1801 | tgcgatgacc ttgatccact gcactgccat ttgatcaggg gttcagaggg cctgggagat |
| 1861 | gggaggaaca cttggggccc tgctatagcc agccagtatt tgctgttcct caggagggac |
| 1921 | taggtggttc cttgaccttc agaactgtgg tgtccttgag gtgagacaac acagtctcta |
| 1981 | aacacagaaa agtgctgaag atcctgcccc caaccgaatt gaccgtgaag gtctggctca |
| 2041 | gtctctgggg ggtgggactc aagctctgga gaggtgggca aaggatgccc attcaacagt |
| 2101 | ccagggttgg ttagaagaga ctgtatgtag ctttgagaaa ctctcccagt attgatgcta |
| 2161 | cactatggat ttcttttctg ggcaatttct tccttccatg tagtatatgt ttgccaatga |
| 2221 | ccactgagat gtgactggaa attttagaat ggtgaagaga tgaacattac ttaaccagat |
| 2281 | cattgggcac agtgattact tgtgactggg tggcaatgat tcagagccct tgtccgttct |
| 2341 | tgcaccctaa gctcccccat atggaatggg ctctcgtttg aagcaaggtt tctagaagat |
| 2401 | gtaggaaggt ctagattctg agaactcttg tgtgtcagaa gagaagcctt gagggctgga |
| 2461 | gtgggctggg ctgcctttga cgcacggcac cagcatgata actgacacat ttctggaaaa |
| 2521 | atcgtttgcc caaagggcag gtctccgtga gcaggaccct cgcgcatgct cggcttccct |
| 2581 | ggattcagct ccatcgctgt ggtccagcag cttgcaacaa aggcctgggt tatttttagt |
| 2641 | cgtcagctcc tgaagaagcc cctggagacc tgggctggct gggcccctct gcccagcggc |
| 2701 | agcatggcct ctgccactcc acaggagtca tcctccccct ggctaattgc tcttggcacg |
| 2761 | tggacccagg gcagcctggc atggaaccaa gcagtgtgac cccccctgca acttctttgc |
| 2821 | agagtgacct gtggcaagag agtgggggtc actttcctgc aggccctgtg gcctcagagc |
| 2881 | tagttccatg catacgaaat gatctcattt aaagggcccc tgtccagaga gcatctgtct |
| 2941 | cctcctctca agctctcttc ttcctcctgc tggttgctgt gcctgtgtgg attcaaaaga |
| 3001 | cccaagggag ggctggagga atggcccgtc tccacggagg ggtacattcc ctctccagac |
| 3061 | tctgcgggct ctctcgttcc acaaaaccca aagcagagta tcttcagaga ctaactactt |
| 3121 | gtttggggga tcatattaaa ttaatttcag aaggg |

SEQ ID NO: 134 Mouse DPF3 Amino Acid Sequence Isoform 1 (NP_001254554.1)

| | |
|---|---|
| 1 | matvihnplk algdqfykea iehcrsynsr lcaersvrlp fldsqtgvaq nncyiwmekr |
| 61 | hrgpglapgq lytyparcwr kkrrlhpped pklrlleikp evelplkkdg ftsesttlea |
| 121 | llrgegvekk vdareeesiq eiqrvlende nveegneeed leedvpkrkn rtrgrargsa |
| 181 | ggrrrhdaas qedhdkpyvc dicgkryknr pglsyhyaht hlaseegdea qdqetrsppn |
| 241 | hrnenhrpqk gpdgtvipnn ycdfclggsn mnkksgrpee lvscadcgrs ghptclqftl |
| 301 | nmteavktyk wqciecksci lcgtsenddq llfcddcdrg yhmycinppv aeppegswsc |
| 361 | hlcwellkek asafgcqa |

SEQ ID NO: 135 Mouse DPF3 cDNA Sequence Variant 2 (NM_001267626.1, CDS:
29-1102)

| | |
|---|---|
| 1 | agacaatatt ctgttacatt gtagcaaaat ggcgactgtc attcacaacc ccctgaaagc |
| 61 | gcttggggac cagttctaca aggaagccat tgagcactgc cggagctaca actcgaggct |
| 121 | gtgcgcagag cggagcgtgc gtctcccctt cctggactcg cagactgggg tggctcagaa |
| 181 | caactgctac atctggatgg agaagaggca ccgcggccca ggcctcgctc cgggccagtt |
| 241 | gtacacatac cctgcccgct gctggcgcaa gaagcgacga ttgcacccac cagaggaccc |
| 301 | aaaactacga ctcctggaaa tcaaacccga agtagaactg cccctgaaga aagatggatt |
| 361 | tacctctgag agtaccacac tggaagcctt gcttcgcggc gagggagtag agaagaaggt |
| 421 | ggatgccaga gaagaggaaa gcatccagga gatacagagg gttttggaaa atgatgaaaa |
| 481 | cgtagaagaa gggaatgaag aggaggattt ggaagaagat gttcccaagc gcaagaacag |
| 541 | gaccagagga cgggctcgcg gctctgcagg cggaaggagg aggcatgatg ccgcctctca |
| 601 | ggaagaccac gacaaaccct acgtctgcga catctgtggc aagcgctaca agaaccggcc |
| 661 | aggactcagc taccactacg ctcatactca cctggccagc gaggagggag acgaagccca |
| 721 | agaccaggag acccgatccc cacccaacca cagaaatgag aaccacagac cccagaaagg |
| 781 | accagacggg acagtcattc ctaataacta ctgtgacttc tgcttggggg gctccaacat |
| 841 | gaacaagaag agtgggaggc ctgaagagct ggtgtcctgt gcagactgtg gacgctctgc |
| 901 | tcatttggga ggagaaggca ggaaggagaa ggaggcagcg gccgcagcac gtaccacgga |
| 961 | ggacttattc ggttccacgt cagaaagtga cacctcaact ttctacggct ttgatgagga |
| 1021 | cgatttggaa gagcctcgct cctgtcgagg acgccgcagt ggccggggtt cacccacagc |
| 1081 | agataaaaag ggcagctgct gagcacatgg gacagactgt gtggccaatt agccacccct |
| 1141 | ccccctgact ctggtcattg ttctagttct gatatatatt tttaaatgaa agacaacttg |
| 1201 | ggcatttccc ttaatccttg ccttttcctt ctgcctccca cgtgtccctc cctctcctag |
| 1261 | cttccttcta ttttgggtac aacagaagca cacactactg agaaccaggg aagagcagga |
| 1321 | tgagagtcct ctggggagcc atggcatcat ggcgggctct tatggactct tatccctaga |
| 1381 | agtaggagaa attaagagga ttttctgtca ctgggggagg gcatcttttt gatgtgagca |
| 1441 | gagttgattt cctgttttca agagaagagg aacatgaggt ttgaaaacaa ataacattaa |
| 1501 | caatatttat ttataaaaaa aaaaaaaaaa aa |

SEQ ID NO: 136 Mouse DPF3 Amino Acid Sequence Isoform 2 (NP_001254555.1)

| | |
|---|---|
| 1 | matvihnplk algdqfykea iehcrsynsr lcaersvrlp fldsqtgvaq nncyiwmekr |
| 61 | hrgpglapgq lytyparcwr kkrrlhpped pklrlleikp evelplkkdg ftsesttlea |
| 121 | llrgegvekk vdareeesiq eiqrvlende nveegneeed leedvpkrkn rtrgrargsa |
| 181 | ggrrrhdaas qedhdkpyvc dicgkryknr pglsyhyaht hlaseegdea qdqetrsppn |
| 241 | hrnenhrpqk gpdgtvipnn ycdfclggsn mnkksgrpee lvscadcgrs ahlggegrke |
| 301 | keaaaaartt edlfgstses dtstfygfde ddleeprscr grrsgrgspt adkkgsc |

SEQ ID NO: 137 Mouse DPF3 cDNA Sequence Variant 3 (NM_058212.2, CDS: 29-
1099)

| | |
|---|---|
| 1 | agacaatatt ctgttacatt gtagcaaaat ggcgactgtc attcacaacc ccctgaaagc |
| 61 | gcttggggac cagttctaca aggaagccat tgagcactgc cggagctaca actcgaggct |
| 121 | gtgcgcagag cggagcgtgc gtctcccctt cctggactcg cagactgggg tggctcagaa |
| 181 | caactgctac atctggatgg agaagaggca ccgcggccca ggcctcgctc cgggccagtt |
| 241 | gtacacatac cctgcccgct gctggcgcaa gaagcgacga ttgcacccac cagaggaccc |
| 301 | aaaactacga ctcctggaaa tcaaacccgt agaactgccc ctgaagaaag atggatttac |
| 361 | ctctgagagt accacactgg aagccttgct tcgcggcgag ggagtagaga agaaggtgga |
| 421 | tgccagagaa gaggaaagca tccaggagat acagagggtt ttggaaaatg atgaaaacgt |
| 481 | agaagaaggg aatgaagagg aggatttgga agaagatgtt cccaagcgca agaacaggac |
| 541 | cagaggacgg gctcgcggct ctgcaggcgg aaggaggagg catgatgccg cctctcagga |
| 601 | agaccacgac aaaccctacg tctgcgacat ctgtggcaag cgctacaaga accggccagg |
| 661 | actcagctac cactacgctc atactcacct ggccagcgag gagggagacg aagcccaaga |
| 721 | ccaggagacc cgatccccac ccaaccacag aaatgagaac cacagacccc agaaaggacc |
| 781 | agacgggaca gtcattccta ataactactg tgacttctgc ttggggggct ccaacatgaa |
| 841 | caagaagagt gggaggcctg aagagctggt gtcctgtgca gactgtggac gctctgctca |
| 901 | tttgggagga gaaggcagga aggagaagga ggcagcggcc gcagcacgta ccacggagga |
| 961 | cttattcggt tccacgtcag aaagtgacac ctcaactttc tacggctttg atgaggacga |
| 1021 | tttggaagag cctcgctcct gtcgaggacg ccgcagtggc cggggttcac ccacagcaga |
| 1081 | taaaaagggc agctgctgag cacatgggac agactgtgtg gccaattagc cacccctccc |
| 1141 | cctgactctg gtcattgttc tagttctgat atatattttt aaatgaaaga caacttgggc |
| 1201 | atttccctta atccttgcct tttccttctg cctcccacgt gtccctccct ctcctagctt |
| 1261 | ccttctattt tgggtacaac agaagcacac actactgaga accagggaag agcaggatga |
| 1321 | gagtcctctg gggagccatg gcatcatggc gggctcttat ggactcttat ccctagaagt |
| 1381 | aggagaaatt aagaggattt tctgtcactg ggggagggca tctttttgat gtgagcagag |
| 1441 | ttgatttcct gttttcaaga gaagaggaac atgaggtttg aaaacaaata acattaacaa |
| 1501 | tatttattta taaaaaaaaa aaaaaaaaa |

SEQ ID NO: 138 Mouse DPF3 Amino Acid Sequence Isoform 3 (NP_478119.1)

| | |
|---|---|
| 1 | matvihnplk algdqfykea iehcrsynsr lcaersvrlp fldsqtgvaq nncyiwmekr |
| 61 | hrgpglapgq lytyparcwr kkrrlhpped pklrlleikp velplkkdgf tsesttleal |
| 121 | lrgegvekkv dareeesiqe iqrvlenden veegneeedl eedvpkrknr trgrargsag |
| 181 | grrrhdaasq edhdkpyvcd icgkryknrp glsyhyahth laseegdeaq dqetrsppnh |
| 241 | rnenhrpqkg pdgtvipnny cdfclggsnm nkksgrpeel vscadcgrsa hlggegrkek |
| 301 | eaaaaartte dlfgstsesd tstfygfded dleeprscrg rrsgrgspta dkkgsc |

SEQ ID NO: 139 Human ACTL6A cDNA Sequence variant 1 (NM_0044301.4, CDS:
214-1503)

| | |
|---|---|
| 1 | agacttaggc ctggacccta gtgattggct gataggagga gccagcaagt gtggctgagc |
| 61 | tccggggtgt gtggacgccg ctttgttgcc tgaggtgggt ggcggtggaa gttaagggag |
| 121 | tcaggggcta tcgctcctcg agactcgcag tcgcggccac tgcagtcact tcgccagtta |
| 181 | gcccttaggg taggagtcgc gccggcagca gccatgagcg gcggcgtgta cgggggagat |
| 241 | gaagttggag cccttgtttt tgacattgga tcctatactg tgagagctgg ttatgctggt |
| 301 | gaggactgcc ccaaggtgga ttttcctaca gctattggta tggtggtaga aagagatgac |
| 361 | ggaagcacat taatggaaat agatggcgat aaaggcaaac aaggcggtcc cacctactac |
| 421 | atagatacta atgctctgcg tgttccgagg gagaatatgg aggccatttc acctctaaaa |
| 481 | aatgggatgg ttgaagactg ggatagtttc caagctattt tggatcatac ctacaaaatg |
| 541 | catgtcaaat cagaagccag tctccatcct gttctcatgt cagaggcacc gtggaatact |
| 601 | agagcaaaga gagagaaact gacagagtta atgtttgaac actacaacat ccctgccttc |
| 661 | ttcctttgca aaactgcagt tttgacagca tttgctaatg gtcgttctac tgggctgatt |
| 721 | ttggacagtg gagccactca taccactgca attccagtcc acgatggcta tgtccttcaa |
| 781 | caaggcattg tgaaatcccc tcttgctgga gactttatta ctatgcagtg cagagaactc |
| 841 | ttccaagaaa tgaatattga attggttcct ccatatatga ttgcatcaaa agaagctgtt |
| 901 | cgtgaaggat ctccagcaaa ctggaaaaga aaagagaagt tgcctcaggt tacgaggtct |
| 961 | tggcacaatt atatgtgtaa ttgtgttatc caggattttc aagcttcggt acttcaagtg |
| 1021 | tcagattcaa cttatgatga acaagtggct gcacagatgc caactgttca ttatgaattc |
| 1081 | cccaatggct acaattgtga ttttggtgca gagcggctaa agattccaga aggattattt |
| 1141 | gacccttcca atgtaaaggg gttatcagga aacacaatgt taggagtcag tcatgttgtc |
| 1201 | accacaagtg ttgggatgtg tgatattgac atcagaccag gtctctatgg cagtgtaata |
| 1261 | gtggcaggag gaaacacact aatacagagt tttactgaca ggttgaatag agagctgtct |
| 1321 | cagaaaactc ctccaagtat gcggttgaaa ttgattgcaa ataatacaac agtggaacgg |
| 1381 | aggtttagct catggattgg cggctccatt ctagcctctt tgggtacctt tcaacagatg |
| 1441 | tggatttcca agcaagaata tgaagaagga gggaagcagt gtgtagaaag aaaatgccct |
| 1501 | tgagaaagag ttcccaagct tctaccttcc ttttgtcacc ttacgtttca tagctttagt |
| 1561 | atactcagga aaagaatgac catcttttgt agaatgttta tacatttttg catatttcaa |
| 1621 | tttccactta aattttttaa agctttaact ggctctataa attaagtttg tgctttcctt |
| 1681 | gaaatgcact tattcttatt acaagcattt tataattttg tataaatgtc tattttctct |
| 1741 | aaatattttg ctttcagtaa aatgctttcc aactctgttt agtgtattaa ttaccagtgg |
| 1801 | attggtagaa ctgcttttta ttgactagta aaagttactg cctatgcttt ttaccttagg |
| 1861 | cttacagaat taaataaaaa ttagccattc cagaaataaa aaaaaaaaaa aaaaaaaaaa |
| 1921 | aaaaaaaaaa aa |

SEQ ID NO: 140 Human ACTL6A Amino Acid Sequence isoform 1 (NP_004292.1)

| | |
|---|---|
| 1 | msggvyggde vgalvfdigs ytvragyage dcpkvdfpta igmvverddg stlmeidgdk |
| 61 | gkqggptyyi dtnalrvpre nmeaisplkn gmvedwdsfq aildhtykmh vkseaslhpv |
| 121 | lmseapwntr akrekltelm fehynipaff lcktavltaf angrstglil dsgathttai |
| 181 | pvhdgyvlqq givksplagd fitmqcrelf qemnielvpp ymiaskeavr egspanwkrk |
| 241 | eklpqvtrsw hnymcncviq dfqasvlqvs dstydeqvaa qmptvhyefp ngyncdfgae |
| 301 | rlkipeglfd psnvkglsgn tmlgvshvvt tsvgmcdidi rpglygsviv aggntliqsf |
| 361 | tdrinrelsq ktppsmrlkl iannttverr fsswiggsil aslgtfqqmw iskqeyeegg |
| 421 | kqcverkcp |

SEQ ID NO: 141 Human ACTL6A cDNA Sequence variant 2 (NM_177989.3; CDS:
196-1359)

| | |
|---|---|
| 1 | agacttaggc ctggacccta gtgattggct gataggagga gccagcaagt gtggctgagc |
| 61 | tccggggtgt gtggacgccg ctttgttgcc tgagatgaag ttggagccct tgtttttgac |
| 121 | attggatcct atactgtgag agctggttat gctggtgagg actgccccaa ggtggatttt |
| 181 | cctacagcta ttggtatggt ggtagaaaga gatgacggaa gcacattaat ggaaatagat |
| 241 | ggcgataaag gcaaacaagg cggtcccacc tactacatag atactaatgc tctgcgtgtt |
| 301 | ccgagggaga atatggaggc catttcacct ctaaaaaatg ggatggttga agactgggat |
| 361 | agtttccaag ctattttgga tcatacctac aaaatgcatg tcaaatcaga agccagtctc |
| 421 | catcctgttc tcatgtcaga ggcaccgtgg aatactagag caaagagaga gaaactgaca |
| 481 | gagttaatgt ttgaacacta caacatccct gccttcttcc tttgcaaaac tgcagttttg |
| 541 | acagcatttg ctaatggtcg ttctactggg ctgattttgg acagtggagc cactcatacc |
| 601 | actgcaattc cagtccacga tggctatgtc cttcaacaag gcattgtgaa atcccctctt |
| 661 | gctggagact ttattactat gcagtgcaga gaactcttcc aagaaatgaa tattgaattg |
| 721 | gttcctccat atatgattgc atcaaaagaa gctgttcgtg aaggatctcc agcaaactgg |
| 781 | aaaagaaaag agaagttgcc tcaggttacg aggtcttggc acaattatat gtgtaattgt |
| 841 | gttatccagg attttcaagc ttcggtactt caagtgtcag attcaactta tgatgaacaa |
| 901 | gtggctgcac agatgccaac tgttcattat gaattcccca atggctacaa ttgtgatttt |
| 961 | ggtgcagagc ggctaaagat tccagaagga ttatttgacc cttccaatgt aaaggggtta |
| 1021 | tcaggaaaca caatgttagg agtcagtcat gttgtcacca caagtgttgg gatgtgtgat |
| 1081 | attgacatca gaccaggtct ctatggcagt gtaatagtgg caggaggaaa cacactaata |
| 1141 | cagagtttta ctgacaggtt gaatagagag ctgtctcaga aaactcctcc aagtatgcgg |
| 1201 | ttgaaattga ttgcaaataa tacaacagtg gaacggaggt ttagctcatg gattggcggc |
| 1261 | tccattctag cctctttggg tacctttcaa cagatgtgga tttccaagca agaatatgaa |
| 1321 | gaaggaggga agcagtgtgt agaaagaaaa tgcccttgag aaagagttcc caagcttcta |
| 1381 | ccttcctttt gtcaccttac gtttcatagc tttagtatac tcaggaaaag aatgaccatc |
| 1441 | ttttgtagaa tgtttataca tttttgcata tttcaatttc cacttaaatt ttttaaagct |
| 1501 | ttaactggct ctataaatta agtttgtgct ttccttgaaa tgcacttatt cttattacaa |
| 1561 | gcattttata attttgtata aatgtctatt ttctctaaat attttgcttt cagtaaaatg |
| 1621 | ctttccaact ctgtttagtg tattaattac cagtggattg gtagaactgc tttttattga |
| 1681 | ctagtaaaag ttactgccta tgctttttac cttaggctta cagaattaaa taaaaattag |
| 1741 | ccattccaga aataaaaaaa aaaaaaaaaa aaaaaaaaaa aaaaaaaa |

SEQ ID NO: 142 Human ACTL6A cDNA Sequence variant 3 (NM_178042.3; CDS:
388-1551)

| | |
|---|---|
| 1 | agacttaggc ctggacccta gtgattggct gataggagga gccagcaagt gtggctgagc |
| 61 | tccggggtgt gtggacgccg ctttgttgcc tgaggtgggt ggcggtggaa gttaagggag |
| 121 | tcaggggcta tcgctcctcg agactcgcag tcgcggccac tgcagtcact tcgccagtta |
| 181 | gcccttaggg taggagtcgc gccggcagca gccatgagcg gcggcgtgta cgggggaggt |
| 241 | gagtgagtgc ggccggacga gagagcgcgc cttttcggcg tgtgggatga agttggagcc |
| 301 | cttgtttttg acattggatc ctatactgtg agagctggtt atgctggtga ggactgcccc |
| 361 | aaggtggatt ttcctacagc tattggtatg gtggtagaaa gagatgacgg aagcacatta |
| 421 | atggaaatag atggcgataa aggcaaacaa ggcggtccca cctactacat agatactaat |
| 481 | gctctgcgtg ttccgaggga gaatatggag gccatttcac ctctaaaaaa tgggatggtt |
| 541 | gaagactggg atagtttcca agctattttg gatcatacct acaaaatgca tgtcaaatca |
| 601 | gaagccagtc tccatcctgt tctcatgtca gaggcaccgt ggaatactag agcaaagaga |
| 661 | gagaaactga cagagttaat gtttgaacac tacaacatcc ctgccttctt cctttgcaaa |
| 721 | actgcagttt tgacagcatt tgctaatggt cgttctactg ggctgatttt ggacagtgga |
| 781 | gccactcata ccactgcaat tccagtccac gatggctatg tccttcaaca aggcattgtg |
| 841 | aaatcccctc ttgctggaga ctttattact atgcagtgca gagaactctt ccaagaaatg |
| 901 | aatattgaat tggttcctcc atatatgatt gcatcaaaag aagctgttcg tgaaggatct |
| 961 | ccagcaaact ggaaaagaaa agagaagttg cctcaggtta cgaggtcttg gcacaattat |
| 1021 | atgtgtaatt gtgttatcca ggattttcaa gcttcggtac ttcaagtgtc agattcaact |
| 1081 | tatgatgaac aagtggctgc acagatgcca actgttcatt atgaattccc caatggctac |
| 1141 | aattgtgatt ttggtgcaga gcggctaaag attccagaag gattatttga cccttccaat |
| 1201 | gtaaaggggt tatcaggaaa cacaatgtta ggagtcagtc atgttgtcac cacaagtgtt |
| 1261 | gggatgtgtg atattgacat cagaccaggt ctctatggca gtgtaatagt ggcaggagga |
| 1321 | aacacactaa tacagagttt tactgacagg ttgaatagag agctgtctca gaaaactcct |
| 1381 | ccaagtatgc ggttgaaatt gattgcaaat aatacaacag tggaacggag gtttagctca |
| 1441 | tggattggcg gctccattct agcctctttg ggtacctttc aacagatgtg gatttccaag |
| 1501 | caagaatatg aagaaggagg gaagcagtgt gtagaaagaa aatgcccttg agaaagagtt |
| 1561 | cccaagcttc taccttcctt ttgtcacctt acgtttcata gctttagtat actcaggaaa |
| 1621 | agaatgacca tcttttgtag aatgtttata catttttgca tatttcaatt tccacttaaa |
| 1681 | ttttttaaag ctttaactgg ctctataaat taagtttgtg ctttccttga aatgcactta |
| 1741 | ttcttattac aagcatttta taattttgta taaatgtcta ttttctctaa atattttgct |
| 1801 | ttcagtaaaa tgctttccaa ctctgtttag tgtattaatt accagtggat tggtagaact |
| 1861 | gctttttatt gactagtaaa agttactgcc tatgcttttt accttaggct tacagaatta |
| 1921 | aataaaaatt agccattcca gaaataaaaa aaaaaaaaaa aaaaaaaaaa aaaaaaaaaa |

SEQ ID NO: 143 Human ACTL6A Amino Acid Sequence isoform 2 (NP_817126.1
and NP_829888.1)

| | |
|---|---|
| 1 | mvverddgst lmeidgdkgk qggptyyidt nalrvprenm eaisplkngm vedwdsfqai |
| 61 | ldhtykmhvk seaslhpvlm seapwntrak rekltelmfe hynipafflc ktavltafan |
| 121 | grstglilds gathttaipv hdgyvlqqgi vksplagdfi tmqcrelfqe mnielvppym |
| 181 | iaskeavreg spanwkrkek lpqvtrswhn ymcncviqdf qasvlqvsds tydeqvaaqm |
| 241 | ptvhyefpng yncdfgaerl kipeglfdps nvkglsgntm lgvshvvtts vgmcdidirp |
| 301 | glygsvivag gntliqsftd rlnrelsqkt ppsmrlklia nnttverrfs swiggsilas |
| 361 | lgtfqqmwis kqeyeeggkq cverkcp |

SEQ ID NO: 144 Mouse ACTL6A cDNA Sequence (NM_019673.2; CDS: 311-1600)

| | |
|---|---|
| 1 | cttcttctgt cgcttctccc tctccctgcc cctacggatg ccttccattg gctaagacgg |
| 61 | ctaaaccgcg cggggatgca gcagcgccac actctgattg gctaatgact aagccggacc |
| 121 | ctttgtcatt ggttgatacg agaaaccagc aagagtggct gtgcagcggg cgtgcggccg |
| 181 | ctgctttgtt gccggagggg gcggcgttgg aagttgcagg cttgcggggc cggcgttctc |
| 241 | agggagagga gtcacgccgc tgttatcttt cgtccggtag tcttcggcca gtccccgcca |
| 301 | gacagtagcc atgagcggcg gcgtgtacgg cggagatgaa gttggcgctc ttgtttttga |
| 361 | cattggatcg tacacagtga gggctggcta tgctggcgag gactgcccta aggttgattt |
| 421 | ccccacggct atcggtgtgg tgctggagag agatgacgga agtacaatga tggagattga |
| 481 | tggtgacaaa ggcaagcagg gcgggcccac ctactacata gacaccaatg ccctccgcgt |
| 541 | gcccagggag aacatggagg ccatctcacc actcaagaat ggcatggttg aagactggga |
| 601 | tagtttccag gccattttgg atcatacata caagatgcat gtcaaatccg aagccagcct |
| 661 | gcatcctgtt ctcatgtcgg aagcaccgtg gaacaccagg gcgaagagag agaaactgac |
| 721 | agagttgatg tttgagcact acagcatccc tgcattcttc ctttgcaaaa ctgcagtttt |
| 781 | gacggcattt gctaatggtc gttctactgg gctgattttg gacagtggag ctacccacac |
| 841 | cactgcgatt ccagtccacg atggctatgt tcttcaacaa ggcattgtga aatcccctct |
| 901 | ggctggagac ttcattacca tgcagtgcag agaactcttc caggaaatga acatagaact |
| 961 | cattcctcct tacatgattg catcaaaaga ggctgttcga gaaggttctc cagccaactg |
| 1021 | gaaaagaaaa gagaaactgc cccaggttac aaggtcttgg cacaattaca tgtgcaactg |
| 1081 | cgtcatccag gattttcaag cttccgttct tcaggtgtca gactccacct acgacgaaca |
| 1141 | agtggctgca cagatgccaa ccgtccacta cgaattcccc aatggctaca actgtgattt |
| 1201 | tggggcagag cggctgaaaa ttcctgaagg gttatttgac ccttccaacg taaagggact |
| 1261 | gtctgggaac acgatgctgg gagtcagtca cgttgtcaca accagcgtcg gaatgtgtga |
| 1321 | catcgacatc agaccaggtc tctacggcag tgtgatcgta gcaggaggaa acacgctaat |
| 1381 | acagagtttc actgacaggt taaatagaga gctttctcag aaaactccac caagtatgcg |
| 1441 | gttgaaactg attgcaaaca acacgacggt ggagcggagg ttcagctcat ggattggtgg |
| 1501 | ctctatccta gcatctttgg gtacctttca acagatgtgg atttctaaac aggaatatga |
| 1561 | agaaggaggg aagcagtgtg tagaaagaaa atgcccttga gggctccacc ctgcctgccc |
| 1621 | gtcacctcaa cgtctgtagc tttagtacac tcaggaaaag atgaccatct tttgtagaat |
| 1681 | gtttatacat gtttgcatat ttcaatttcc acttaaattt tttaaggctt taactggctc |
| 1741 | tataaattaa atgagtttgt gctttccttg aaatgcactt attcttatta caggcatttt |
| 1801 | ataattttgt atgaatgtct attttctcta aatattttgc tttcagtaag tactctccag |
| 1861 | ctctcctggg ggttggttgg tggaattact ctgtattgac aagtacaagt tactgcctat |
| 1921 | gctttgtacc ttaggctaca aaactaaata aaaatcacta ctgtcctag |

SEQ ID NO: 145 Mouse ACTL6A Amino Acid Sequence (NP_062647.2)

| | |
|---|---|
| 1 | msggvyggde vgalvfdigs ytvragyage dcpkvdfpta igvvlerddg stmmeidgdk |
| 61 | gkqggptyyi dtnalrvpre nmeaisplkn gmvedwdsfq aildhtykmh vkseaslhpv |
| 121 | lmseapwntr akrekltelm fehysipaff lcktavltaf angrstglil dsgathttai |
| 181 | pvhdgyvlqq givksplagd fitmqcrelf qemnielipp ymiaskeavr egspanwkrk |
| 241 | eklpqvtrsw hnymcncviq dfqasvlqvs dstydeqvaa qmptvhyefp ngyncdfgae |
| 301 | rlkipeglfd psnvkglsgn tmlgvshvvt tsvgmcdidi rpglygsviv aggntliqsf |
| 361 | tdrinrelsq ktppsmrlkl iannttverr fsswiggsil aslgtfqqmw iskqeyeegg |
| 421 | kqcverkcp |

SEQ ID NO: 146 Human β-Actin cDNA Sequence (NM_001101.4; CDS: 193-1320)

| | |
|---|---|
| 1 | gagtgagcgg cgcggggcca atcagcgtgc gccgttccga aagttgcctt ttatggctcg |
| 61 | agcggccgcg gcggcgccct ataaaaccca gcggcgcgac gcgccaccac cgccgagacc |
| 121 | gcgtccgccc cgcgagcaca gagcctcgcc tttgccgatc cgccgcccgt ccacacccgc |
| 181 | cgccagctca ccatggatga tgatatcgcc gcgctcgtcg tcgacaacgg ctccggcatg |
| 241 | tgcaaggccg gcttcgcggg cgacgatgcc ccccgggccg tcttcccctc catcgtgggg |
| 301 | cgccccaggc accagggcgt gatggtgggc atgggtcaga aggattccta tgtgggcgac |
| 361 | gaggcccaga gcaagagagg catcctcacc ctgaagtacc ccatcgagca cggcatcgtc |
| 421 | accaactggg acgacatgga gaaaatctgg caccacacct tctacaatga gctgcgtgtg |
| 481 | gctcccgagg agcaccccgt gctgctgacc gaggcccccc tgaaccccaa ggccaaccgc |
| 541 | gagaagatga cccagatcat gtttgagacc ttcaacaccc cagccatgta cgttgctatc |
| 601 | caggctgtgc tatccctgta cgcctctggc cgtaccactg gcatcgtgat ggactccggt |
| 661 | gacggggtca cccacactgt gcccatctac gaggggtatg ccctccccca tgccatcctg |
| 721 | cgtctggacc tggctggccg ggacctgact gactacctca tgaagatcct caccgagcgc |
| 781 | ggctacagct tcaccaccac ggccgagcgg gaaatcgtgc gtgacattaa ggagaagctg |
| 841 | tgctacgtcg ccctggactt cgagcaagag atggccacgg ctgcttccag ctcctccctg |
| 901 | gagaagagct acgagctgcc tgacggccag gtcatcacca ttggcaatga gcggttccgc |
| 961 | tgccctgagg cactcttcca gccttccttc ctgggcatgg agtcctgtgg catccacgaa |
| 1021 | actaccttca actccatcat gaagtgtgac gtggacatcc gcaaagacct gtacgccaac |
| 1081 | acagtgctgt ctggcggcac caccatgtac cctggcattg ccgacaggat gcagaaggag |
| 1141 | atcactgccc tggcacccag cacaatgaag atcaagatca ttgctcctcc tgagcgcaag |
| 1201 | tactccgtgt ggatcggcgg ctccatcctg gcctcgctgt ccaccttcca gcagatgtgg |
| 1261 | atcagcaagc aggagtatga cgagtccggc ccctccatcg tccaccgcaa atgcttctag |
| 1321 | gcggactatg acttagttgc gttacaccct ttcttgacaa aacctaactt gcgcagaaaa |
| 1381 | caagatgaga ttggcatggc tttatttgtt ttttttgttt tgttttggtt tttttttttt |
| 1441 | ttttggcttg actcaggatt taaaaactgg aacggtgaag gtgacagcag tcggttggag |
| 1501 | cgagcatccc ccaaagttca caatgtggcc gaggactttg attgcacatt gttgtttttt |
| 1561 | taatagtcat tccaaatatg agatgcgttg ttacaggaag tcccttgcca tcctaaaagc |
| 1621 | caccccactt ctctctaagg agaatggccc agtcctctcc caagtccaca caggggaggt |
| 1681 | gatagcattg ctttcgtgta aattatgtaa tgcaaaattt ttttaatctt cgccttaata |
| 1741 | cttttttatt ttgttttatt ttgaatgatg agccttcgtg cccccccttc cccctttttt |
| 1801 | gtcccccaac ttgagatgta tgaaggcttt tggtctccct gggagtgggt ggaggcagcc |
| 1861 | agggcttacc tgtacactga cttgagacca gttgaataaa agtgcacacc ttaaaaatga |
| 1921 | ggaaaaaaaa aaaaaaaaaa |

SEQ ID NO: 147 Human β-Actin Amino Acid Sequence (NP_001092.1)

| | |
|---|---|
| 1 | mdddiaalvv dngsgmckag fagddaprav fpsivgrprh qgvmvgmgqk dsyvgdeaqs |
| 61 | krgiltlkyp iehgivtnwd dmekiwhhtf ynelrvapee hpvllteapl npkanrekmt |
| 121 | qimfetfntp amyvaiqavl slyasgrttg ivmdsgdgvt htvpiyegya lphailrldl |
| 181 | agrdltdylm kiltergysf tttaereivr dikeklcyva ldfeqemata assssleksy |
| 241 | elpdgqviti gnerfrcpea lfqpsflgme scgihettfn simkcdvdir kdlyantvls |
| 301 | ggttmypgia drmqkeital apstmkikii apperkysvw iggsilasls tfqqmwiskq |
| 361 | eydesgpsiv hrkcf |

SEQ ID NO: 148 Mouse β-Actin cDNA Sequence (NM_007393.5; CDS: 110-1237)

| | |
|---|---|
| 1 | tataaaaccc ggcggcgcaa cgcgcagcca ctgtcgagtc gcgtccaccc gcgagcacag |
| 61 | cttctttgca gctccttcgt tgccggtcca cacccgccac cagttcgcca tggatgacga |
| 121 | tatcgctgcg ctggtcgtcg acaacggctc cggcatgtgc aaagccggct tcgcgggcga |
| 181 | cgatgctccc cgggctgtat tcccctccat cgtgggccgc cctaggcacc agggtgtgat |
| 241 | ggtgggaatg ggtcagaagg actcctatgt gggtgacgag gcccagagca agagaggtat |
| 301 | cctgaccctg aagtacccca ttgaacatgg cattgttacc aactgggacg acatggagaa |
| 361 | gatctggcac cacaccttct acaatgagct gcgtgtggcc cctgaggagc accctgtgct |
| 421 | gctcaccgag gcccccctga accctaaggc caaccgtgaa aagatgaccc agatcatgtt |
| 481 | tgagaccttc aacaccccag ccatgtacgt agccatccag gctgtgctgt ccctgtatgc |
| 541 | ctctggtcgt accacaggca ttgtgatgga ctccggagac ggggtcaccc acactgtgcc |
| 601 | catctacgag ggctatgctc tccctcacgc catcctgcgt ctggacctgg ctggccggga |
| 661 | cctgacagac tacctcatga agatcctgac cgagcgtggc tacagcttca ccaccacagc |
| 721 | tgagagggaa atcgtgcgtg acatcaaaga gaagctgtgc tatgttgctc tagacttcga |
| 781 | gcaggagatg gccactgccg catcctcttc ctccctggag aagagctatg agctgcctga |
| 841 | cggccaggtc atcactattg gcaacgagcg gttccgatgc cctgaggctc ttttccagcc |
| 901 | ttccttcttg ggtatggaat cctgtggcat ccatgaaact acattcaatt ccatcatgaa |
| 961 | gtgtgacgtt gacatccgta aagacctcta tgccaacaca gtgctgtctg gtggtaccac |
| 1021 | catgtaccca ggcattgctg acaggatgca gaaggagatt actgctctgg ctcctagcac |
| 1081 | catgaagatc aagatcattg ctcctcctga gcgcaagtac tctgtgtgga tcggtggctc |
| 1141 | catcctggcc tcactgtcca ccttccagca gatgtggatc agcaagcagg agtacgatga |
| 1201 | gtccggcccc tccatcgtgc accgcaagtg cttctaggcg gactgttact gagctgcgtt |
| 1261 | ttacaccctt tctttgacaa aacctaactt gcgcagaaaa aaaaaaaata agagacaaca |
| 1321 | ttggcatggc tttgtttttt taaatttttt ttaaagtttt tttttttttt tttttttttt |
| 1381 | tttttaagtt tttttgtttt gttttggcgc ttttgactca ggatttaaaa actggaacgg |
| 1441 | tgaaggcgac agcagttggt tggagcaaac atcccccaaa gttctacaaa tgtggctgag |
| 1501 | gactttgtac attgttttgt tttttttttt ttttggtttt gtcttttttt aatagtcatt |
| 1561 | ccaagtatcc atgaaataag tggttacagg aagtccctca ccctcccaaa agccaccccc |
| 1621 | actcctaaga ggaggatggt cgcgtccatg ccctgagtcc accccgggga aggtgacagc |
| 1681 | attgcttctg tgtaaattat gtactgcaaa aattttttta aatcttccgc cttaatactt |
| 1741 | catttttgtt tttaatttct gaatggccca ggtctgaggc ctcccttttt tttgtccccc |
| 1801 | caacttgatg tatgaaggct ttggtctccc tgggaggggg ttgaggtgtt gaggcagcca |
| 1861 | gggctggcct gtacactgac ttgagaccaa taaaagtgca caccttacct tacacaaaca |
| 1921 | aaaaaaaaaa aaaaa |

SEQ ID NO: 149 Mouse β-Actin Amino Acid Sequence (NP_031419.1)

| | |
|---|---|
| 1 | mdddiaalvv dngsgmckag fagddaprav fpsivgrprh qgvmvgmgqk dsyvgdeaqs |
| 61 | krgiltlkyp iehgivtnwd dmekiwhhtf ynelrvapee hpvllteapl npkanrekmt |
| 121 | qimfetfntp amyvaiqavl slyasgrttg ivmdsgdgvt htvpiyegya lphailrldl |
| 181 | agrdltdylm kiltergysf tttaereivr dikeklcyva ldfeqemata assssleksy |
| 241 | elpdgqviti gnerfrcpea lfqpsflgme scgihettfn simkcdvdir kdlyantvls |
| 301 | ggttmypgia drmqkeital apstmkikii apperkysvw iggsilasls tfqqmwiskq |
| 361 | eydesgpsiv hrkcf |

SEQ ID NO: 150 Human BCL7A cDNA Sequence variant 1 (NM_020993.4; CDS:
207-902)

| | |
|---|---|
| 1 | actgggccag gcgcgcggcg gccccgggct ttgtgtgtgt gtgtatgtgt gtgtgtgtgt |
| 61 | gtgtgtgtgt gtgagtgtgt gcgtgtgaga gtgcgagtgt ctgtgcgcga gtgagtgagc |
| 121 | ggcgggcggg cgcgagtgtg gccgccgcgg agcgcgagca ggacccggcg ggcgcgctcc |
| 181 | ccagcctccg tctccccgcc ggaaccatgt cgggcaggtc ggttcgagcc gagacgagga |
| 241 | gccgggccaa agatgatatc aagagggtca tggcggcgat cgagaaagtg cgcaaatggg |
| 301 | agaagaaatg ggtgaccgtt ggtgacacat ccctacgaat ctacaaatgg gtccctgtga |
| 361 | cggagcccaa ggttgatgac aaaaacaaga ataagaaaaa aggcaaggac gagaagtgtg |
| 421 | gctcagaggt gaccactccg gagaacagtt cctccccagg gatgatggac atgcatgacg |
| 481 | ataacagcaa ccagagctcc atcgcagatg cctcccccat caaacaggag aacagcagca |
| 541 | actccagccc cgctccagag cccaactcgg ctgtgcccag cgacggcacc gaggccaagg |
| 601 | tggatgaggc ccaggctgat gggaaggagc acccaggagc tgaagatgct tctgatgagc |
| 661 | agaattcaca gtcctcgatg gaacattcga tgaacagctc agagaaagta gatcggcagc |
| 721 | cgtctggaga ctcgggtctg gccgcagaga cgtctgcaat ctctcaggta cctcgctcga |
| 781 | ggtctcagag gggcagccag atcggccggg agcccattgg gttgtcgggg gatttggaag |
| 841 | gagtgccacc ctctaaaaag atgaaactgg aggcctctca acaaaactcc gaagagatgt |
| 901 | agacgatgct ttaaagcctc cgatccatgt tccatggaag gtacatcagc aattaattct |
| 961 | agagcaactt tgccccagcg attcctcttg ggtgcgaaca gaactactaa cgtttcaagt |
| 1021 | ttaccaagtg caaatccaag aagacccaga acggcgtcac ttctcagaca ctgaagaact |
| 1081 | ctgctgtgaa gcaaaacact caaaccttta agggactgtc cttggggagg caggcggggc |
| 1141 | tgacagctca ggagtgtctg cacactgtct cggaagccag gattccattt gtgttgctgc |
| 1201 | tgtattttcc ccccacttct ctatgtaacg atataagcta tcggagggtg gtaccgatca |
| 1261 | ggaacgcttt ttggcggggc tttccactgt tcaaccgatt ccttccgctt tctttttttg |
| 1321 | tgccttgtgc ccttgaggtg acctctggca tgtatcctgg tggttcttac atccccctct |
| 1381 | gcaaagtgcc ctcttggttt ggttcgggcg gcggctgcca ccctactcac cgctctcctc |
| 1441 | cctgccccag gacttcatcg gagcaggcag ggtggagcga aggagctcct tagcccacct |
| 1501 | ggtttgcagg tgcaggggga ccttaggcac gccccaagca ccaggcacca gggcccaagg |
| 1561 | acgcgcaggt gttggggcac agtccccaag ggctcggccc cttggatcag gctgggcact |
| 1621 | cgctgtgctc tcccctcctt ggggcgttta ggactgggcg tctccaagcc caccatggcc |
| 1681 | cagatggacg tgcaaagccc ttggaatttt ctggcacttc ctctctattg cccccaccac |
| 1741 | caccaccccc atcactgctt tctcccagac ctccgaatac gaaatggctt ctctggctga |
| 1801 | ctgcaaggct gtctccttaa ggcactgagt gggccgggga ggctgggagc cggcggcagg |
| 1861 | attagctggt gctgaacttt ctctcatagg acgtcgcttg gatttcaaat ccacggtcac |
| 1921 | ctgctgccct ttgcctcccc cgacgcccca gcctgtgccc cggagaggca ggatcgcagt |
| 1981 | ggtcagaatc cacgtgcttt cctattctca ggctgttctg actctgagcc aacagctgga |
| 2041 | ccgtgtctca tccccagaac atgccgtctg tccccaccgg ggagtgggcc ttgatggccg |
| 2101 | ggcctcgaag gccacaaaca aggcgtcgag gaattggaaa gatttgcaca ccctccagaa |
| 2161 | aggagagacg caatctcccc tccctcccat cccccacctt cgctggaaca gcttcctctc |
| 2221 | actgaacgga gacgccccct tggacgaact gcctaatcgt ttggttctga ggcctggttt |
| 2281 | gctcttaatt aatatatgaa ctcctcagac cttaaacctt ttcctaagct ttctttactg |
| 2341 | cactggagtt ctgactccct ttgagttgtg tgttactggg ggtggggtgg ggtcatgggt |
| 2401 | tttgttgttt ttgggggcta attggtgcat attcaggtac cacctttgac gtgtggctct |
| 2461 | ttctcctgac catcatggga agtgtctgct ggattccatt ttctaagagt ttctgagggt |
| 2521 | gaggctctta tttttttttt taagggatcc tgtctatttc ctgcacttcg agaagaatca |
| 2581 | aaatgttcct gaatttcaaa tacctcatgc aaaatgtctc ctgaaataag ggaaaaaaaa |
| 2641 | aaaaccacaa ctttgaaaat cttaatgttg aagttagcaa tgccgaaagg tttctgtctt |
| 2701 | aaaaaaaaaa atccttgtac ttatcaattt tgccccttag gcagtcagtt ttgttgagaa |
| 2761 | ctgtgtcctg catcctggcg cagaacctac ctgatgcggt tcctctccac gcatctcgag |
| 2821 | gcggcgttac ctccagattc cgtagagtta gagtcacatt tttctttgca gcgaaactcc |
| 2881 | atcttggtga gagatgaatt tggatattta tttccttctc tgtttttggg aaacgagagg |
| 2941 | ctacaaccaa gacagctgaa ggagaatgaa acacacacat ccacagaaac agagaggcgt |
| 3001 | aggtggccct gccgttgacc gcagcctctc tggacaggca aggggagttg gcgcaggtga |
| 3061 | ggactcagac gacgtccacc gtcccaaggc tgtcactagt atttctctga agtgcctgaa |
| 3121 | ggtaggaatg ggccggcgat tgggaccagc tgggccccac cacggccacg ccaggcaaag |
| 3181 | cgccagcagc cctgcactcc acgctggcca agaaggcctt ccacgcagaa tgacaagact |
| 3241 | gcaaaaatcc gatgtgcttc cttccctggc gcagtcgctc ctcgagccgc tgccccccac |
| 3301 | ccaccctgca cccctcgccc tccccccacc acagaatcta agacctttca gcttcgagcc |
| 3361 | agggggcggg ggatcccgag caaaagcctt ccgtggacat caggccccgt ggcctcaagg |
| 3421 | gctcccaggg caaacctaat tccccccaaa acgtgaagtc ggggaagctg cggctacaca |
| 3481 | ttccacaaag tgctggcact tacacccaca acccggaagg ctgtggaccg attcctctag |
| 3541 | ggtggtgacc tcccattagc aaacggtgtc atggtttgga atgttcatta tcgccaagaa |
| 3601 | cctggttaga ggcataaaga ccttttttca ccgttaccta attttttccc ctttcaagaa |
| 3661 | tttttttttt ttttggtgtg ttgtacagca gtataatttt tcacttattt attccatcag |
| 3721 | tagatatggt ttgtacaatg tacaattgtt tcatttcaga aaataaaaat ttcaaatcat |
| 3781 | gaa |

SEQ ID NO: 151 Human BCL7A Amino Acid Sequence isoform A (NP_066273.1)

| | |
|---|---|
| 1 | msgrsvraet rsrakddikr vmaaiekvrk wekkwvtvgd tslriykwvp vtepkvddkn |
| 61 | knkkkgkdek cgsevttpen ssspgmmdmh ddnsnqssia daspikqens snsspapepn |
| 121 | savpsdgtea kvdeaqadgk ehpgaedasd eqnsqssmeh smnssekvdr qpsgdsglaa |
| 181 | etsaisqvpr srsqrgsqig repiglsgdl egvppskkmk leasqqnsee m |

SEQ ID NO: 152 Human BCL7A cDNA Sequence variant 2 (NM_001024808.2; CDS:
207-839)

| | |
|---|---|
| 1 | actgggccag gcgcgcggcg gccccgggct ttgtgtgtgt gtgtatgtgt gtgtgtgtgt |
| 61 | gtgtgtgtgt gtgagtgtgt gcgtgtgaga gtgcgagtgt ctgtgcgcga gtgagtgagc |
| 121 | ggcgggcggg cgcgagtgtg gccgccgcgg agcgcgagca ggacccggcg ggcgcgctcc |
| 181 | ccagcctccg tctccccgcc ggaaccatgt cgggcaggtc ggttcgagcc gagacgagga |
| 241 | gccgggccaa agatgatatc aagagggtca tggcggcgat cgagaaagtg cgcaaatggg |
| 301 | agaagaaatg ggtgaccgtt ggtgacacat ccctacgaat ctacaaatgg gtccctgtga |
| 361 | cggagcccaa ggttgatgac aaaaacaaga ataagaaaaa aggcaaggac gagaagtgtg |
| 421 | gctcagaggt gaccactccg gagaacagtt cctccccagg gatgatggac atgcatgacg |
| 481 | ataacagcaa ccagagctcc atcgcagatg cctcccccat caaacaggag aacagcagca |
| 541 | actccagccc cgctccagag cccaactcgg ctgtgcccag cgacggcacc gaggccaagg |
| 601 | tggatgaggc ccaggctgat gggaaggagc acccaggagc tgaagatgct tctgatgagc |
| 661 | agaattcaca gtcctcgatg gaacattcga tgaacagctc agagaaagta gatcggcagc |
| 721 | cgtctggaga ctcgggtctg gccgcagaga cgtctgcaat ctctcaggat ttggaaggag |
| 781 | tgccaccctc taaaaagatg aaactggagg cctctcaaca aaactccgaa gagatgtaga |
| 841 | cgatgcttta aagcctccga tccatgttcc atggaaggta catcagcaat taattctaga |
| 901 | gcaactttgc cccagcgatt cctcttgggt gcgaacagaa ctactaacgt ttcaagttta |
| 961 | ccaagtgcaa atccaagaag acccagaacg gcgtcacttc tcagacactg aagaactctg |
| 1021 | ctgtgaagca aaacactcaa acctttaagg gactgtcctt ggggaggcag gcggggctga |
| 1081 | cagctcagga gtgtctgcac actgtctcgg aagccaggat tccatttgtg ttgctgctgt |
| 1141 | attttccccc cacttctcta tgtaacgata taagctatcg gagggtggta ccgatcagga |
| 1201 | acgctttttg gcggggcttt ccactgttca accgattcct tccgctttct ttttttgtgc |
| 1261 | cttgtgccct tgaggtgacc tctggcatgt atcctggtgg ttcttacatc cccctctgca |
| 1321 | aagtgccctc ttggtttggt tcgggcggcg gctgccaccc tactcaccgc tctcctccct |
| 1381 | gccccaggac ttcatcggag caggcagggt ggagcgaagg agctccttag cccacctggt |
| 1441 | ttgcaggtgc agggggacct taggcacgcc ccaagcacca ggcaccaggg cccaaggacg |
| 1501 | cgcaggtgtt ggggcacagt ccccaagggc tcggcccctt ggatcaggct gggcactcgc |
| 1561 | tgtgctctcc cctccttggg gcgtttagga ctgggcgtct ccaagcccac catggcccag |
| 1621 | atggacgtgc aaagcccttg gaattttctg gcacttcctc tctattgccc ccaccaccac |
| 1681 | cacccccatc actgctttct cccagacctc cgaatacgaa atggcttctc tggctgactg |
| 1741 | caaggctgtc tccttaaggc actgagtggg ccggggaggc tgggagccgg cggcaggatt |
| 1801 | agctggtgct gaactttctc tcataggacg tcgcttggat ttcaaatcca cggtcacctg |
| 1861 | ctgccctttg cctcccccga cgccccagcc tgtgccccgg agaggcagga tcgcagtggt |
| 1921 | cagaatccac gtgctttcct attctcaggc tgttctgact ctgagccaac agctggaccg |
| 1981 | tgtctcatcc ccagaacatg ccgtctgtcc ccaccgggga gtgggccttg atggccgggc |
| 2041 | ctcgaaggcc acaaacaagg cgtcgaggaa ttggaaagat ttgcacaccc tccagaaagg |
| 2101 | agagacgcaa tctcccctcc ctcccatccc ccaccttcgc tggaacagct tcctctcact |
| 2161 | gaacggagac gcccccttgg acgaactgcc taatcgtttg gttctgaggc ctggtttgct |
| 2221 | cttaattaat atatgaactc ctcagacctt aaaccttttc ctaagctttc tttactgcac |
| 2281 | tggagttctg actccctttg agttgtgtgt tactgggggt ggggtggggt catgggtttt |
| 2341 | gttgtttttg ggggctaatt ggtgcatatt caggtaccac ctttgacgtg tggctctttc |
| 2401 | tcctgaccat catgggaagt gtctgctgga ttccattttc taagagtttc tgagggtgag |
| 2461 | gctcttattt ttttttttaa gggatcctgt ctatttcctg cacttcgaga agaatcaaaa |
| 2521 | tgttcctgaa tttcaaatac ctcatgcaaa atgtctcctg aaataaggga aaaaaaaaaa |
| 2581 | accacaactt tgaaaatctt aatgttgaag ttagcaatgc cgaaaggttt ctgtcttaaa |
| 2641 | aaaaaaaatc cttgtactta tcaattttgc cccttaggca gtcagttttg ttgagaactg |
| 2701 | tgtcctgcat cctggcgcag aacctacctg atgcggttcc tctccacgca tctcgaggcg |
| 2761 | gcgttacctc cagattccgt agagttagag tcacattttt ctttgcagcg aaactccatc |
| 2821 | ttggtgagag atgaatttgg atatttattt ccttctctgt ttttgggaaa cgagaggcta |
| 2881 | caaccaagac agctgaagga gaatgaaaca cacacatcca cagaaacaga gaggcgtagg |
| 2941 | tggccctgcc gttgaccgca gcctctctgg acaggcaagg ggagttggcg caggtgagga |
| 3001 | ctcagacgac gtccaccgtc ccaaggctgt cactagtatt tctctgaagt gcctgaaggt |
| 3061 | aggaatgggc cggcgattgg gaccagctgg gccccaccac ggccacgcca ggcaaagcgc |
| 3121 | cagcagccct gcactccacg ctggccaaga aggccttcca cgcagaatga caagactgca |
| 3181 | aaaatccgat gtgcttcctt ccctggcgca gtcgctcctc gagccgctgc cccccaccca |
| 3241 | ccctgcaccc ctcgccctcc ccccaccaca gaatctaaga cctttcagct tcgagccagg |
| 3301 | gggcggggga tcccgagcaa aagccttccg tggacatcag gccccgtggc ctcaagggct |
| 3361 | cccagggcaa acctaattcc ccccaaaacg tgaagtcggg gaagctgcgg ctacacattc |
| 3421 | cacaaagtgc tggcacttac acccacaacc cggaaggctg tggaccgatt cctctagggt |
| 3481 | ggtgacctcc cattagcaaa cggtgtcatg gtttggaatg ttcattatcg ccaagaacct |
| 3541 | ggttagaggc ataaagacct tttttcaccg ttacctaatt ttttcccctt tcaagaattt |
| 3601 | tttttttttt tggtgtgttg tacagcagta taatttttca cttatttatt ccatcagtag |
| 3661 | atatggtttg tacaatgtac aattgtttca tttcagaaaa taaaaatttc aaatcatgaa |

SEQ ID NO: 153 Human BCL7A Amino Acid Sequence isoform B (NP_001019979.1)

| | |
|---|---|
| 1 | msgrsvraet rsrakddikr vmaaiekvrk wekkwvtvgd tslriykwvp vtepkvddkn |
| 61 | knkkkgkdek cgsevttpen ssspgmmdmh ddnsnqssia daspikqens snsspapepn |
| 121 | savpsdgtea kvdeaqadgk ehpgaedasd eqnsqssmeh smnssekvdr qpsgdsglaa |
| 181 | etsaisqdle gvppskkmkl easqqnseem |

SEQ ID NO: 154 Mouse BCL7A cDNA Sequence (NM_029850.3; CDS: 183-815)

| | |
|---|---|
| 1 | ttgcgcactg ggccccgggc gcgcggcggc accaggcttt gtgtgtgcgc gtatgtgtgt |
| 61 | gagtgtgtgt ctgtgcgcga gtgagagagc gggcgagtgt ggcgagcagg acccggcggg |
| 121 | cgcgctcccc cagcctccct ctctctctct ctttcctctc tctctccctc cccgccagaa |
| 181 | ccatgtcggg caggtcggtt cgagccgaga ccaggagccg ggccaaagat gatatcaaga |
| 241 | gggtcatggc ggctatcgag aaagtgcgca aatgggagaa gaaatgggtg accgttggcg |
| 301 | atacatccct acgaatctac aagtgggtcc ctgtgacgga gccaaaggtt gatgataaaa |
| 361 | acaagaacaa gaagaaaggc aaggacgaga agtgtggctc ggaggtgacc actccagaga |
| 421 | acagctcgtc tcctgggatg atggacatgc acgatgataa cagcaaccag agctccatag |
| 481 | cagacgcctc ccccatcaag caagagaaca gcagcaactc cagccctgcc ccagagacca |
| 541 | acccacccgt gcccagcgat ggcaccgaag ccaaggctga tgaggcgcag gccgatggaa |
| 601 | aagagcaccc tggagctgaa gatgcatccg aggagcaaaa ttcacagtct tcgatggaaa |
| 661 | actcggtgaa cagctccgag aaggcagaac ggcagccatc tgcagaatca gggttagcgg |
| 721 | cagaaacgtc ggcagtctct caggatttgg aaggagtgcc gccgtctaaa aagatgaagc |
| 781 | tggaagcctc tcaacagaac tcagaagaga tgtagacggc ccggcggaac cttctggtcc |
| 841 | atgtttcatg gcaggtacat cggcaggctt aattctagaa acacggccca agcgactcct |
| 901 | cttgggcgcg agcagaacta acgtttcaag tttactaaag tgcaaatcca agaagaacct |
| 961 | agagcggcgg cggcagcgga acttcgcaga cacttgacgg actctgccgt gaaaccgaaa |
| 1021 | cactcgaacc ttcaagtgac tgccctctgg gaggtgggtc gacagctcag gagtgtgtgc |
| 1081 | gcactgtctc ggaagccaag attacatttg tgttgctgct gtatccccct cccctcactt |
| 1141 | ctctatttaa cgatataagc tattcgaggg tggtaccaat caggaatttg ctttccatag |
| 1201 | gggcttttgg ctcttcaacc aattccttct gctttctttt tttgtgcctt gtaccctaga |
| 1261 | ggtgacctcc ggcatgcttc ctggtttttg catctctcct ggcaaagtgc ccacttgttt |
| 1321 | tggttggctg ctgcccccac ccccacccct tattgcctct ctcctccctg ccccaagact |
| 1381 | gcttcaaagc aagcagggta gagcggcggg agaccaggca cctttcagtg acccccttgg |
| 1441 | ttcaggtgag cagtgtttgg gcacaccctg agccccaact tccagggccc ctggggctac |
| 1501 | aagtttgcgg gggccggttt cccgagggct ggcctccttg gtcaggacac gccctcacct |
| 1561 | tttggagcca tggaggctag gcgtttgcaa ggcaaggtag cccagattga catgcaaaag |
| 1621 | cctttagatt tttctggcac ttccacccta tctcccctcc gccccctaac ctcacacccc |
| 1681 | gactctggcc acaactggca ctgcgctctc caggtcctcc gaagacgaaa tgaccaactg |
| 1741 | agcttgtctc cttaggatag taaagggctg ggaggttggg agccggcggc cggcaggaat |
| 1801 | agctggtgct gaactaactc tcccatagga cattgcttgg atttcaaatc catggtaacc |
| 1861 | tgctgccctt tgtccctgtc tcctatccac cgcaccccaa gccccccaaa accccaggca |
| 1921 | ggatgcgcct ggtatggcct gactctgaga ggctacaggt ggatggagac ccattcccag |
| 1981 | taccgcgctg ttggtctcct ctggggaccg gaccttaacc attgggcctc aggccagaag |
| 2041 | caaggcacag aggaaccggg aagatttgca cacagatttg cccccccaga aaggagcctc |
| 2101 | cgaggcactt ccttcccctg ctcttccttg cacggagaca gctctctctc actcagtgga |
| 2161 | gacgccactt ggacagacgg actgctcagc tgttgatttc tgaggcctgg tttgctctta |
| 2221 | atccctttgc tggacccctc agatctgaaa accttcccct atgctttctt actgcactgg |
| 2281 | agttcgaact ccctatgagt tgtgtgttgg ggggaggggc gggcggggtg ggttttgttt |
| 2341 | ttttgttgtt cttgtttcgt tttgtttcgt ttgctaattg gtgcatattc aggtaccacc |
| 2401 | ttttgacgtg tggatctttc tccaaaccac cacaagaagt gtctgccggg ctccgttttc |
| 2461 | taagagtttc tgaggggaca gctcccattt ctttttttgg tttcaaggga gctgtctatt |
| 2521 | tcctatactt caagaagaat caaaatgttc ctgaatttta aatacctcat gcaaaaatat |
| 2581 | ctcctgaaat aagggaaaaa aaaaaaactt tgaaaaatcg taatgttgaa gttagcgatg |
| 2641 | ctaaaatgtt tctgtcttaa aaaacaaaaa aattgttgta atacttagcg attttgcccc |
| 2701 | tcaggcggtc agttctgtcc agaactgtgt tctgcgtctt ggcccggaag caaccggatg |
| 2761 | catgacctct gaacggatct caaggccaag gcatctttac ctccagattc tagagttagg |
| 2821 | gcaacaacag ttttcttttg cagcaaaact ccgttctggt gaaagatgaa tttggatatt |
| 2881 | tatttctttt tctgggaaac aagaggttaa acaacgtaag cagctgaggg agaacccaac |
| 2941 | acgggcatcc acggaaccag cgggcgcggc cagggccgcc tatacctctt ctaccctccg |
| 3001 | cagcctctct ggacagtcag gaggagtcga tacagttgag aaagaagaca acgatgaggt |
| 3061 | tcgaggtacc gaggctgtca ttagtttttc tctgaagtgc ctgaacgtag gaatgggccg |
| 3121 | tcgacggagg ggaccattcg gatgttcccc cacctcgcga cggccgcgcc aggcaaagag |
| 3181 | ccagcagccc tgcactccac actggccagg aaaagccttc cacgaggagc ggtcagactg |
| 3241 | caaaatccaa tgtgcttcct tccccgccac ggtcctctct ctctctcggg gagccgatgg |
| 3301 | tccccgtccc tgaaccccct agcccgcatc cccaccacag aatctaagac ctttcatctg |
| 3361 | gccgagccag gggcaaaggg gatcctaagc aaatgccttc cgtggacaac aggccccacg |
| 3421 | gcctaaaggg ctcccagggc aaactttccc ccaacacttg aaggggggtg ggggggatgg |
| 3481 | cggctacaca ttccactaag tgcagcactc gcacccacaa cccggaagga aggctcttaa |
| 3541 | gcgattctca gagggtggtg actgcccatc atcgtcagac ggtgtcgtgg tttggaatgt |
| 3601 | taattatcgc agaggacctg gtagaggtat aaagaccttt tttcactgtt acctaatttt |
| 3661 | ttttttcctc ttacaatttt ttttttggtg tgttgtacag cagtataatt tttcacttat |
| 3721 | ttattccatc ggtagatatt gtttgtacaa tgtacaatgg tttcatttca gaaaataata |
| 3781 | ataataaaaa aaaaagttct gatcatgag |

SEQ ID NO: 155 Mouse BCL7A Amino Acid Sequence (NP_084126.1)

| | |
|---|---|
| 1 | msgrsvraet rsrakddikr vmaaiekvrk wekkwvtvgd tslriykwvp vtepkvddkn |
| 61 | knkkkgkdek cgsevttpen ssspgmmdmh ddnsnqssia daspikqens snsspapetn |
| 121 | ppvpsdgtea kadeaqadgk ehpgaedase eqnsqssmen svnssekaer qpsaesglaa |
| 181 | etsavsqdle gvppskkmkl easqqnseem |

SEQ ID NO: 156 Human BCL7B cDNA Sequence variant 1 (NM_001707.3; CDS:
158-766)

| | |
|---|---|
| 1 | gcgggcgggt gcgcgcgctt tctcgcgcac gcgcgcacgg agggggcgac ggccgctgtg |
| 61 | acgctgcggc ggcggcgggc gggcggcggc gcgtgaggcg cgcgatcccc ggtgtcttgg |
| 121 | gagcagtgcc ccggcccccg ccgctcccgc cgccgccatg tcgggccggt cggtccgggc |
| 181 | ggagacccgc agccgggcca aggacgacat caagaaggtg atggcggcca tcgagaaagt |
| 241 | gcggaaatgg gagaagaagt gggtgactgt gggtgacacg tccctgagga tatttaagtg |
| 301 | ggttcctgtg acagacagca aggagaaaga aaagtcaaaa tcgaacagtt cagcagcccg |
| 361 | agaacctaat ggctttcctt ctgatgcctc agccaattcc tctctccttc ttgaattcca |
| 421 | ggacgaaaac agcaaccaga gttccgtgtc tgacgtctat cagcttaagg tggacagcag |
| 481 | caccaactca agccccagcc cccagcagag tgagtccctg agcccagcac acacctccga |
| 541 | cttccgcacg gatgactccc agcccccaac gctgggccag gagatcctgg aggagccctc |
| 601 | cctgccctcc tcggaagttg ctgatgaacc tcctaccctc accaaggaag aaccagttcc |
| 661 | actagagaca caggtcgttg aggaagagga agactcaggt gccccgcccc tgaagcgctt |
| 721 | ctgtgtggac caacccacag tgccgcagac ggcgtcagaa agctagcacc atcccggccc |
| 781 | tccgcctcct ggccctgcct ctatttattg cattctggtt ctggccgcgc cgcgttgctg |
| 841 | gggtaagggc aagcactggg gtcaagagcc tgcacacatg agccttccgg gctggaaggc |
| 901 | tggcgtagga cttggggctg tagcatcatc ttcctgaccc tggcacctgt gtctacttgc |
| 961 | tcccgagaag aggagcgctc atgtcttttt tgcaccccaa gttggctgga gcatcggcca |
| 1021 | ccccaagatt catctgtgac ctccaggcag cagtctctgc tccagaatct ctggacggag |
| 1081 | ctgctggcag cttctgcgag aagagagaga tgtggaaggc accttctaga agagagcgtg |
| 1141 | cctcaggtta cttgaacttg aacggagact gtagactccc ggactttccc ctaggactgg |
| 1201 | gggccctgta ggctgctgtt ggaggactgg gtagagacat tggagggaag ggaagggctt |
| 1261 | ttctccacac aagggcagag agtccgtcta gatttcttgc tgtcctgcca gctctgccca |
| 1321 | tgcctgaggt ggtcctacct ctcacgggca ccctagctgc tgacagccct ttgtggccgc |
| 1381 | cgtccccatc ccctgccctc agcacacaca tctgcacaca cgcagctttg ttctcacctc |
| 1441 | tacctgtcat tccagcatcc ctgcctcttg tcacaaactg ccccagcaag aatttgaggt |
| 1501 | tctgacaaca gtacccatcc cccacagtac cccttcagct cagtttctag aaagctccct |
| 1561 | tttctttgaa atctgcatgt tgaattgaac tttgtgattt tattttttgt ttcaaaaaag |
| 1621 | tttaagaaaa tggaaatggg caacagtgag tgaagacata ttttagcact gaatagaata |
| 1681 | tttttaaaat taaactattt gaaatatgtc caaaaaaaaa aaaaaaaaa |

SEQ ID NO: 157 Human BCL7B Amino Acid Sequence isoform 1 (NP_001698.2)

| | |
|---|---|
| 1 | msgrsvraet rsrakddikk vmaaiekvrk wekkwvtvgd tslrifkwvp vtdskekeks |
| 61 | ksnssaarep ngfpsdasan sslllefqde nsnqssvsdv yqlkvdsstn sspspqqses |
| 121 | lspahtsdfr tddsqpptlg qeileepslp ssevadeppt ltkeepvple tqvveeeeds |
| 181 | gapplkrfcv dqptvpqtas es |

SEQ ID NO: 158 Human BCL7B cDNA Sequence variant 2 (NM_001197244.1; CDS:
158-595)

| | |
|---|---|
| 1 | gcgggcgggt gcgcgcgctt tctcgcgcac gcgcgcacgg agggggcgac ggccgctgtg |
| 61 | acgctgcggc ggcggcgggc gggcggcggc gcgtgaggcg cgcgatcccc ggtgtcttgg |
| 121 | gagcagtgcc ccggcccccg ccgctcccgc cgccgccatg tcgggccggt cggtccgggc |
| 181 | ggagacccgc agccgggcca aggacgacat caagaaggtg atggcggcca tcgagaaagt |
| 241 | gcggaaatgg gagaagaagt gggtgactgt gggtgacacg tccctgagga tatttaagtg |
| 301 | ggttcctgtg acagacagca aggagaaaga aaagtcaaaa tcgaacagtt cagcagcccg |
| 361 | agaacctaat ggctttcctt ctgatgcctc agccaattcc tctctccttc ttgaattcca |
| 421 | ggagccctcc ctgccctcct cggaagttgc tgatgaacct cctaccctca ccaaggaaga |
| 481 | accagttcca ctagagacac aggtcgttga ggaagaggaa gactcaggtg ccccgcccct |
| 541 | gaagcgcttc tgtgtggacc aacccacagt gccgcagacg gcgtcagaaa gctagcacca |
| 601 | tcccggccct ccgcctcctg gccctgcctc tatttattgc attctggttc tggccgcgcc |
| 661 | gcgttgctgg ggtaagggca agcactgggg tcaagagcct gcacacatga gccttccggg |
| 721 | ctggaaggct ggcgtaggac ttggggctgt agcatcatct tcctgaccct ggcacctgtg |
| 781 | tctacttgct cccgagaaga ggagcgctca tgtctttttt gcaccccaag ttggctggag |
| 841 | catcggccac cccaagattc atctgtgacc tccaggcagc agtctctgct ccagaatctc |
| 901 | tggacggagc tgctggcagc ttctgcgaga agagagagat gtggaaggca ccttctagaa |
| 961 | gagagcgtgc ctcaggttac ttgaacttga acggagactg tagactcccg gactttcccc |
| 1021 | taggactggg ggccctgtag gctgctgttg gaggactggg tagagacatt ggagggaagg |
| 1081 | gaagggcttt tctccacaca agggcagaga gtccgtctag atttcttgct gtcctgccag |
| 1141 | ctctgcccat gcctgaggtg gtcctacctc tcacgggcac cctagctgct gacagccctt |
| 1201 | tgtggccgcc gtccccatcc cctgccctca gcacacacat ctgcacacac gcagctttgt |
| 1261 | tctcacctct acctgtcatt ccagcatccc tgcctcttgt cacaaactgc cccagcaaga |
| 1321 | atttgaggtt ctgacaacag tacccatccc ccacagtacc ccttcagctc agtttctaga |
| 1381 | aagctccctt ttctttgaaa tctgcatgtt gaattgaact ttgtgatttt attttttgtt |
| 1441 | tcaaaaaagt ttaagaaaat ggaaatgggc aacagtgagt gaagacatat tttagcactg |
| 1501 | aatagaatat ttttaaaatt aaactatttg aaatatgtcc aaaaaaaaaa aaaaaaaa |

SEQ ID NO: 159 Human BCL7B Amino Acid Sequence isoform 2 (NP_001184173.1)

| | |
|---|---|
| 1 | msgrsvraet rsrakddikk vmaaiekvrk wekkwvtvgd tslrifkwvp vtdskekeks |
| 61 | ksnssaarep ngfpsdasan sslllefqep slpssevade pptltkeepv pletqvveee |
| 121 | edsgapplkr fcvdqptvpq tases |

SEQ ID NO: 160 Human BCL7B cDNA Sequence variant 3 (NM_001301061.1; CDS:
247-888)

| | |
|---|---|
| 1 | gcgggcgggt gcgcgcgctt tctcgcgcac gcgcgcacgg agggggcgac ggccgctgtg |
| 61 | acgctgcggc ggcggcgggc gggcggcggc gcgtgaggcg cgcgatcccc ggtgtcttgg |
| 121 | gagcagtgcc ccggcccccg ccgctcccgc cgccgccatg tcgggccggt cggtccgggc |
| 181 | ggagacccgc agccgggcca aggacgacat caagaaggtg atggcggcca tcgagaaagt |
| 241 | gcggaaatga cggaatctcg ctctgtcacc caggctggag tgcattggcg caatctcggc |
| 301 | tcactgcaac ctctgcctct caggttcaag caattctcct gcctcagcct cctgagtagc |
| 361 | tgggactaca gggagaagaa gtgggtgact gtgggtgaca cgtccctgag gatatttaag |
| 421 | tgggttcctg tgacagacag caaggagaaa gaaaagtcaa aatcgaacag ttcagcagcc |
| 481 | cgagaaccta atggctttcc ttctgatgcc tcagccaatt cctctctcct tcttgaattc |
| 541 | caggacgaaa acagcaacca gagttccgtg tctgacgtct atcagcttaa ggtggacagc |
| 601 | agcaccaact caagccccag cccccagcag agtgagtccc tgagcccagc acacacctcc |
| 661 | gacttccgca cggatgactc ccagccccca acgctgggcc aggagatcct ggaggagccc |
| 721 | tccctgccct cctcggaagt tgctgatgaa cctcctaccc tcaccaagga agaaccagtt |
| 781 | ccactagaga cacaggtcgt tgaggaagag gaagactcag gtgccccgcc cctgaagcgc |
| 841 | ttctgtgtgg accaacccac agtgccgcag acggcgtcag aaagctagca ccatcccggc |
| 901 | cctccgcctc ctggccctgc ctctatttat tgcattctgg ttctggccgc gccgcgttgc |
| 961 | tggggtaagg gcaagcactg gggtcaagag cctgcacaca tgagccttcc gggctggaag |
| 1021 | gctggcgtag gacttggggc tgtagcatca tcttcctgac cctggcacct gtgtctactt |
| 1081 | gctcccgaga agaggagcgc tcatgtcttt tttgcacccc aagttggctg gagcatcggc |
| 1141 | caccccaaga ttcatctgtg acctccaggc agcagtctct gctccagaat ctctggacgg |
| 1201 | agctgctggc agcttctgcg agaagagaga gatgtggaag gcaccttcta gaagagagcg |
| 1261 | tgcctcaggt tacttgaact tgaacggaga ctgtagactc ccggactttc ccctaggact |
| 1321 | gggggccctg taggctgctg ttggaggact gggtagagac attggaggga agggaagggc |
| 1381 | ttttctccac acaagggcag agagtccgtc tagatttctt gctgtcctgc cagctctgcc |
| 1441 | catgcctgag gtggtcctac ctctcacggg caccctagct gctgacagcc ctttgtggcc |
| 1501 | gccgtcccca tcccctgccc tcagcacaca catctgcaca cacgcagctt tgttctcacc |
| 1561 | tctacctgtc attccagcat ccctgcctct tgtcacaaac tgccccagca agaatttgag |
| 1621 | gttctgacaa cagtacccat cccccacagt accccttcag ctcagtttct agaaagctcc |
| 1681 | cttttctttg aaatctgcat gttgaattga actttgtgat tttatttttt gtttcaaaaa |
| 1741 | agtttaagaa aatggaaatg ggcaacagtg agtgaagaca tattttagca ctgaatagaa |
| 1801 | tatttttaaa attaaactat ttgaaatatg tccaaaaaaa aaaaaaaaaa a |

SEQ ID NO: 161 Human BCL7B Amino Acid Sequence isoform 3 (NP_001287990.1)

| | |
|---|---|
| 1 | mtesrsvtqa gvhwrnlgsl qplplrfkqf sclsllsswd yrekkwvtvg dtslrifkwv |
| 61 | pvtdskekek sksnssaare pngfpsdasa nsslllefqd ensnqssvsd vyqlkvdsst |
| 121 | nsspspqqse slspahtsdf rtddsqpptl gqeileepsl pssevadepp tltkeepvpl |
| 181 | etqvveeeed sgapplkrfc vdqptvpqta ses |

SEQ ID NO: 162 Mouse BCL7B cDNA Sequence (NM_009745.2; CDS: 136-744)

| | |
|---|---|
| 1 | acgcgcgcac ggaggggggg cgacggccgc ggtgacgtgc tgcggtggca gcgggtggac |
| 61 | ggcgacgcgt gaggcgcgtg atatcccgcg tcttgggagc actgtcccgg cccccagcca |
| 121 | ctccccgccg ccgccatgtc cggccgttcg gtccgggccg agacccgtag ccgggctaaa |
| 181 | gatgacatca agaaggtgat ggcggccatc gagaaagtgc ggaaatggga gaagaaatgg |
| 241 | gtgactgtgg gtgatacctc cctgaggata ttcaaatggg tgcctgtgac agatagcaag |
| 301 | gagaaagaaa agtcaaaatc gaataataca gcagcccggg aacctaatgg ctttccctct |
| 361 | gacgcctcag ccaattcctc cctcctcctt gaattccagg atgagaacag caaccagagc |
| 421 | tctgtgtcgg atgtctatca actcaaggtg gacagcagca ccaactcaag tcccagcccc |
| 481 | cagcagagcg agtccctgag cccagcacac acctcagact tccgcactga tgactcccag |
| 541 | ccccccacat tgggccagga gatcctggag gaaccttcgc tgcctgcatc tgaagttgca |
| 601 | gatgaacctc ccacactcac aaaggaagag ccagtgccgg tggagacaca gaccactgag |
| 661 | gaagaggagg actctggtgc tccgcccttg aagagattct gtgtggacca acctgtagta |
| 721 | ccgcagacca cgtcggaaag ctagcaccgt cctggcccct cgcctcctgg cccctgcctc |
| 781 | tatttattgc attctggtct ggccgagctc tgatgctggg gtccgggcaa gcactagggt |
| 841 | ccagagcctg tgcgtgggag ccctctgggc tagaaggctg atggagggcg tggggtcgtc |
| 901 | gcaccatctt cttgttcctg acacttgtgt ctgcttgctc ttgagcaaag gagcgctcac |
| 961 | atcttttctg tagcccaagt aggccagagc atcagggttc atttctcacc tccagaacca |
| 1021 | ctgcacggag ctgctggcgc cgccacgggg agaaaggtgt ggaaggcgcc cacctgagag |
| 1081 | aagagtgcct aggattactt gaattgaatg gagactgtgg agtatggact ttgccacagg |
| 1141 | gccaggccct gcaggctgct gctgggagag ggactgaccg gtagagatgt ggagaacacc |
| 1201 | ggagagaggc tcttccggga cggaggggct ttcgccacct ttgggcagaa gacccatggg |
| 1261 | agatgcatcc tgtgcctgag gcagacctgc ctctgttgga tgccccagct gctcccagcc |
| 1321 | ctgtgcctgc cagaaccttc tgctgcatcc tcacactcac taagcacacc tgaagctttc |
| 1381 | tattcacccg tcctttcatt ccaacgtccc cacctcctcc tgcagaaaac cccagccatg |
| 1441 | attggaggtt ctgaccacag tacctgcccc agtactcctt cagctcagac tttctagaaa |
| 1501 | gttccttttt ctttaaaatc tgcatgttta attaaacttt atgattttat tttttgtctg |
| 1561 | aaaaaagaaa agtttaagaa aatggaaatg ggtaacagca agtgaagacc tattttagca |
| 1621 | ctgaatagag tatttttaaa attaaacttt gaaatatgtc ttgttaaaaa aaaaaaaa |

SEQ ID NO: 163 Mouse BCL7B Amino Acid Sequence (NP_033875.2)

| | |
|---|---|
| 1 | msgrsvraet rsrakddikk vmaaiekvrk wekkwvtvgd tslrifkwvp vtdskekeks |
| 61 | ksnntaarep ngfpsdasan sslllefqde nsnqssvsdv yqlkvdsstn sspspqqses |
| 121 | lspahtsdfr tddsqpptlg qeileepslp asevadeppt ltkeepvpve tqtteeeeds |
| 181 | gapplkrfcv dqpvvpqtts es |

SEQ ID NO: 164 Human BCL7C cDNA Sequence variant 1 (NM_001286526.1; CDS:
359-1087)

| | |
|---|---|
| 1 | tccgtcccca actcgcgcgt ccgtccccaa ctcccgctct cggcggcggg cagggggcgc |
| 61 | tgagcgtcca ggcgctccaa gggggcgggc ccgggtcggg gcggggccgg ccgggcttcc |
| 121 | aggcctgggc tctggccgcc cgcgccaccg ggccgctccg gggacaggcc ggggcggggc |
| 181 | gcggcggcag gaaacggggc ggggacttgc ggaggcgttg gggacgagag agggcgcggc |
| 241 | caactccagg ggggacggca ggccgagagc gcggcgcccg ggcctggcgc ggagcctgag |
| 301 | cccgccggac gggaggcggc cccgccgcgg gctcggcccc ggccccagcc ccgccagcat |
| 361 | ggccggccgg actgtacggg ccgagacccg gagccgggcc aaggatgaca tcaagaaggt |
| 421 | gatggcgacc atcgagaagg tccggagatg ggagaagcga tgggtgactg tgggcgacac |
| 481 | ttcccttcgt atcttcaagt gggtgccagt ggtggatccc caggaggagg agcgaaggcg |
| 541 | ggcaggtggc ggggcagaga gatcccgtgg ccgggaacgt cggggcaggg gcgccagtcc |
| 601 | ccgagggggt ggccctctca tcctgctgga tcttaatgat gagaacagca accagagttt |
| 661 | ccattcggaa ggttccctgc aaaagggcac agagcccagt cctgggggca ccccccagcc |
| 721 | cagccgccct gtgtcacctg ccggaccccc agaaggggtc cctgaggagg ctcagccccc |
| 781 | acggctgggc caagagagag atcccggggg cataactgct ggcagcaccg acgaaccccc |
| 841 | aatgctgacc aaggaggagc ctgttccaga actgctggaa gctgaggatt cgggagtgag |
| 901 | aatgacgagg agagcccttc acgagaaggg gctgaagaca gagcccctca ggaggctcct |
| 961 | gcccaggagg ggcctccgga caaatgtccg gcccagttcc atggcggtgc cggacaccag |
| 1021 | agctcccggg ggaggcagca aggccccgag ggcacccaga acaatccccc agggtaaggg |
| 1081 | gaggtgagtg ggctccccaa gcaagccaag acccctaaag cctcccttgg ctgccccaag |
| 1141 | atccagccac tacctgtgcc ccgagggcgg aaagagcttc ccagctcacc caccgcggta |
| 1201 | acatcggagg gcgagcggcc ccacacctgc ccgaacctaa ggccacagca cccatctggc |
| 1261 | tcgccactgg cgcccgaatg catgggaagg gcttagggca gaactcggac cacatccagt |
| 1321 | gcctgaggcc gccttgctag aggcctaggg gaggggtgca ctgggctgcc tcgcccacct |
| 1381 | cctcacgcac ccatgcggcc accctcccag cggtctgagt gtgccatgcg aggcgcctgc |
| 1441 | caccccggga gaggcgccga gtcccgagtc ctgccggcac tgagcctccg ggtccacagc |
| 1501 | gggcaagggc cgtggcgggg acaagcgcag gggacccgcc ggcctcccgc cttctgcagc |
| 1561 | accacgagat gcccacgtgg cacctggacg tccatgcata tgttgaggcc cgtgcacgcg |
| 1621 | cagagacccc agcgcagaag ccgccccgca cgccagggct tatgtatgcc agcgctggga |
| 1681 | gacctccagc gcccgaggac atacggcaag tggttccacc agggtgtcag cctagcaggc |
| 1741 | caacctggga acccatgtgg acaagcggcc tttcagccca ggcgcccgcc tcgggtggag |
| 1801 | gcgtggagac ttctggcgca gccctgagct ggtggcctaa cctacctgga aaatcctagc |
| 1861 | ccgagaagca gcgcgagtga gccttttggg tggttccaag gcccttcacc aagctctcac |
| 1921 | ttcctgactt caccgttggg tctgttgtac taggaaataa taacgcctcc catttatcaa |
| 1981 | gggtttactc tgtaaaaa |

SEQ ID NO: 165 Human BCL7C Amino Acid Sequence isoform 1 (NP_001273455.1)

| | |
|---|---|
| 1 | magrtvraet rsrakddikk vmatiekvrr wekrwvtvgd tslrifkwvp vvdpqeeerr |
| 61 | ragggaersr grerrgrgas prgggplill dlndensnqs fhsegslqkg tepspggtpq |
| 121 | psrpvspagp pegvpeeaqp prlgqerdpg gitagstdep pmltkeepvp elleaedsgv |
| 181 | rmtrralhek glkteplrrl lprrglrtnv rpssmavpdt rapgggskap raprtipqgk |
| 241 | gr |

SEQ ID NO: 166 Human BCL7C cDNA Sequence variant 2 (NM_004765.3; CDS:
359-1012)

| | |
|---|---|
| 1 | tccgtcccca actcgcgcgt ccgtccccaa ctcccgctct cggcggcggg cagggggcgc |
| 61 | tgagcgtcca ggcgctccaa gggggcgggc ccgggtcggg gcggggccgg ccgggcttcc |
| 121 | aggcctgggc tctggccgcc cgcgccaccg ggccgctccg gggacaggcc ggggcggggc |
| 181 | gcggcggcag gaaacggggc ggggacttgc ggaggcgttg gggacgagag agggcgcggc |
| 241 | caactccagg ggggacggca ggccgagagc gcggcgcccg ggcctggcgc ggagcctgag |
| 301 | cccgccggac gggaggcggc cccgccgcgg gctcggcccc ggccccagcc ccgccagcat |
| 361 | ggccggccgg actgtacggg ccgagacccg gagccgggcc aaggatgaca tcaagaaggt |
| 421 | gatggcgacc atcgagaagg tccggagatg ggagaagcga tgggtgactg tgggcgacac |
| 481 | ttcccttcgt atcttcaagt gggtcccagt ggtggatccc caggaggagg agcgaaggcg |
| 541 | ggcaggtggc ggggcagaga gatcccgtgg ccgggaacgt cggggcaggg gcgccagtcc |
| 601 | ccgagggggt ggccctctca tcctgctgga tcttaatgat gagaacagca accagagttt |
| 661 | ccattcggaa ggttccctgc aaaagggcac agagcccagt cctgggggca ccccccagcc |
| 721 | cagccgccct gtgtcacctg ccggaccccc agaaggggtc cctgaggagg ctcagccccc |
| 781 | acggctgggc caagagagag atcccggggg cataactgct ggcagcaccg acgaaccccc |
| 841 | aatgctgacc aaggaggagc ctgttccaga actgctggaa gctgaggccc ccgaagctta |
| 901 | ccctgtcttt gagccagtgc cacctgtccc tgaggcagcc cagggtgaca cagaggactc |
| 961 | ggagggtgcc cccccactca agcgcatctg cccaaatgcc cctgacccct gagaagccgg |
| 1021 | cctgcctgtc ctgttgcccc aggggcccct ttggcttttt acaaataaag acccttttgt |
| 1081 | aaaaaaaaaa aaaaaaaaaa a |

SEQ ID NO: 167 Human BCL7C Amino Acid Sequence isoform 2 (NP_004756.2)

| | |
|---|---|
| 1 | magrtvraet rsrakddikk vmatiekvrr wekrwvtvgd tslrifkwvp vvdpqeeerr |
| 61 | ragggaersr grerrgrgas prgggplill dlndensnqs fhsegslqkg tepspggtpq |
| 121 | psrpvspagp pegvpeeaqp prlgqerdpg gitagstdep pmltkeepvp elleaeapea |
| 181 | ypvfepvppv peaaqgdted segapplkri cpnapdp |

SEQ ID NO: 168 Mouse BCL7C cDNA Sequence variant 1 (NM_001347652.1; CDS:
240-965)

| | |
|---|---|
| 1 | ggccggggct ctagcagccc gcgccgcccg ggccgctccg gggacgggcc ggggcggggc |
| 61 | gcggtcttag gaagccaggc ggggacgcgc ggaggcgttg gggagcgagg gagggcgcgg |
| 121 | ccaactcccg gagggacggc aggccgaaag agcggcgctg gggcctggcg ctcagcctga |
| 181 | gatcgccgga ccacaggccg ccccgccacg ggctctgtcc cggccccagc cccgccagca |
| 241 | tggccggccg gaccgtgcgg gccgagaccc ggagccgggc caaagatgac atcaagaagg |
| 301 | tgatggcgac catcgagaag gtccggagat gggagaagcg ctgggtgact gtgggagaca |
| 361 | cttcccttcg aatcttcaag tgggtgcctg tggtggatcc ccaggaggag gagaggcggc |
| 421 | gggcaggagg cggggcagag agatcccgtg gccgggagag acgtggtagg ggcaccagtc |
| 481 | ccagaggggg aggccccctc atcctactgg atctcaatga tgagaacagc aaccagagtt |
| 541 | tccattctga aggttcattg caaaagggtg ctgagcccag ccctgggggg acgccccagc |
| 601 | ccagccgccc tggatcacca actggacccc cagaagtgat tactgaagat actcagcccc |
| 661 | cacaattggg tcaggagaga gatccagggg ggacacctgc aggcggtact gatgaacccc |
| 721 | caaagctgac caaggaggag cctgttccag aattgctaga agctgaggat tccggcgtga |
| 781 | gactcaccag gagagccctt caagagaaag gcctgaaaac cgagcccctc aggaggcttc |
| 841 | tccccaggag aggcctccgg acaaattctc ggccaacttc cacggttccg gaacccagag |
| 901 | ctcccggaag tgggagcaag gcccagaggg cacccaggac gataccacaa gggaagggga |
| 961 | ggtgagcggg ttccaccaca caaggggagg cccttaggtc ttccttagct gcctcaagat |
| 1021 | ccagtcattt acccacaccg tttaagggtg gagagggctt tggagctggg cacccgcagc |
| 1081 | cagcaatgga ggtcggcagc cagctctctg cttgtccctg tccctaaatt atggatccat |
| 1141 | cctgcttgct gtgggtccaa aactactggg ccagagcagg tcccagacag ggaatgtctg |
| 1201 | gggacatctc taggtgatgc ctagaagcaa cttgaataca caaaatggtg gatcctatgc |
| 1261 | caacttggtc acctcctcac acacttaggg cagccatcca ccaaagggcc aggcatggcc |
| 1321 | cctggaggtg accttcgacc tttggaacta cagtatctac actggtgagg ggccctacca |
| 1381 | gcaagacttg agcagcgagc aacccctgaa gcactgggca aaaggtaatg ccacagcttg |
| 1441 | tgaatggtgt gaagattcaa ttgcccgtgt gtagagacac cactccagca agcacctggc |
| 1501 | agcctcaccc gcttccacga gcctatggac tctgggcctg ctaattaacc cttggctcca |
| 1561 | gaagacatgt gccaaccagg gtgccaacct tgcctcaggt caatcgaggg gtgcacatgg |
| 1621 | cccagtgacc tttcagacca ggccacagcc tcctgcccca ggaatggatg gagacatgtg |
| 1681 | gtccagcact gccaaatcta cctggaaact acccactttg agaaactcat ggcagatgag |
| 1741 | ccatctgagt gattccaagg gctttcatca acctcttgcc tccgacttga caactcactt |
| 1801 | ggccaggagg tagtgtctcc tgtaccacag agagctaact gtactacata ttgcaacttg |
| 1861 | tgggacttat ttaatgcagc actcctgtca tagatcctgt tactttcaca ttttacagat |
| 1921 | atagaaaaca agcaaccagg aggttaaaga gcttgcccca agtcacacag cctgtctgtg |
| 1981 | gcagagccag cattcacatc cagtctaccc acctgactcc acagtccctg ctagtgtacc |
| 2041 | actttttgtg ctgggcatgc aggtgggctg cagctgtgag ctttgttgag gcgttcattg |
| 2101 | aaggaacatc catttttctc agtggcaaat tacaaaggac ttttaatttt aactttcttc |
| 2161 | tgcctgacct accttccttc cttccttcct tccttccttc cttccttcct tccttccttc |
| 2221 | cttccttcct tcctttcttc ctctgctggc catgaaccca ctagaccagg ccagtcttga |
| 2281 | actcacagag gtctgtctat ctctgcctct ccagtgctgg gattaaaggt gagcgacacc |
| 2341 | atacccagct taggccttct ttgtttgttt gtttgttgtt ttttgagtaa taaggtaagc |
| 2401 | agatgttctg tgtccataac tgagatgaca tggacattga gtggtaaggg acttgagctc |
| 2461 | agcccctggg tccctcagat tcctctctgg agtgccattg atacaggaag catcatctag |
| 2521 | gcccagctcc tgattggcga cttcccagaa gccatgggct gtcatgccaa gtgactgggg |
| 2581 | aacttcaagt aacaaacatt tattaattag acttctgaac taccaatggg gcagaagttt |
| 2641 | tcacgtttca aacacagata ctagttttca agattcagaa atgaaacata ggaattctgg |
| 2701 | ggaggtccag aaagtcctac tttgtatttt tcataactct ctgtatctta aaagctaaga |
| 2761 | aactcacagt tcatcgtagt ttaaaagagc tgcaagcctt aaatattcaa aaggtagaaa |
| 2821 | ctgccagtgt gtgtcactgg gtagtagttg aataacaaaa tgtttacgga tccaattaga |
| 2881 | ttcatggtac tccagagtca tgagttgaaa tcgcggatat aaagacttat ttccaatgca |
| 2941 | tcatttctca gaacaccctg ggatttgtat aaaacacacg atgcatgtga acgcattcat |
| 3001 | gtttatctta tttctgagaa tcattctaca ggcgggggag cacgcataca tttttaatgt |
| 3061 | cagggctaca gaagactggc ctggcacggc tcccctcagt tcttggttcc caaattctaa |
| 3121 | ggatgtctgc cttcgtttca tgtgtcagcc tttcctgctc tcggacctga cacagtggct |
| 3181 | ccgtacagcg aggactcctc tgtgctgatg aacttcggct gttagaggac tgttagtatg |
| 3241 | tttcctgttt cgccaattta tttgctgatt ggttttgtga ttcaaaaaac aaacaagcaa |
| 3301 | gcaaacaaac aaaaacaaaa gcagggacca ggcgtggtgg cgcacgcctt taatcttgga |
| 3361 | ggcagaggca ggcggatttc tgagttcgag gccaacctga tctacaaagt gagttccagg |
| 3421 | acagccaggg ctatacagag aaaccctgtc tcaaaaacaa acaaacaaaa acaaacaaaa |
| 3481 | aaacaaaagc agacaaaatc accaccagca gcagcaacaa tcccaggttt cccaataatg |
| 3541 | tcagcaagga attctgaaca gacaaagtcc gtggggctga gcagggacgg tgaataagtg |
| 3601 | agctcgtgtt tatgaagccc agtgatctgc tccttgcagc cagaacgctc cagctcagcc |
| 3661 | aggccctggc acgagccctc ggctgaagca ctcacctctg agcttcagtt tagtgagtag |
| 3721 | catcctccct agaaagtaat attcttgctt catacggtga tatggtggaa gggttaccag |
| 3781 | catggctttg gagtcagaca gactgtggtt caaatcttag ctacacgact ttctacctct |
| 3841 | ttgatttggg gcaagttcta accgctggct ttttctcttc tgtaaaatga ggacatggaa |
| 3901 | tctatttcac agggctgtgg cttcagtgag atcacatatg acccgcttaa gtcaaagcgg |
| 3961 | gtccacggta tgtgtttgat cccacgtagg cattacccgc tgtatctacc tcacagggca |
| 4021 | gttgtgagga tgaagggtag agggaaatgc tttccaaact gtgaagtgat ctgtgtttac |
| 4081 | ctctctcctc tggagatgga gagataggaa gttgctgtca gacactagtg ggatgcccat |
| 4141 | ggagagggcc tagtatgctt ctgtgcacac agtgtggctg ggctgaaggg gaggtgctgt |
| 4201 | gttgtgcagt ggtgcacagc gggggcgtgc cctccggtga gggttgctgc actgaagtgg |
| 4261 | ggaagttcag tgcctatggc tacactgttg ggagcaggga gagcgcaggt cctatcttaa |
| 4321 | gaaggatgct agatgggggc taaagtagat gagtgtttgc ctagcatgag caagggccat |
| 4381 | ggatttcgta tctagcacct caggaaaaac acaacaaaca aacaaacaaa caaacccctc |
| 4441 | ttcttgttta aagattctgg ataaagaaca gtgttgtgaa cgtgtgtatc cactgtttgt |
| 4501 | ctttttaaat acaactcaaa tagcaggaag gcctgtgtgc acaagaggtg acaagtgact |
| 4561 | gcaagtgttt ccatcgctgg cagccatgca ccctcctacc acgagtacag atttcattct |
| 4621 | ggagtgtgca gaccaaatgc aggtcagagg gccctcccgg ggcaactcgc caagatcctg |
| 4681 | accaaagcct agcctcacaa agtaatccct agcccagtta gcagatcagg ggttggggct |
| 4741 | tgggaacgtc atgtccaatg tccaaggctg cacaggtcct gtggggacag aatccaagcc |
| 4801 | cttcacctgg attggggttc ctccgcctgc cagtctcaga tctctgatct tgaacaagga |
| 4861 | tagcatgcag aagagtaagg ttccatgcct aagtgacctc ctctggacct cagacgcagt |
| 4921 | tcttgctcct gacctcatgc ctcgtctcca gacatcactc cccagcttag cccttaggtc |
| 4981 | aggctcctct gggcaccatc cttagattca acccaaagga gggtcctctc attctaacca |
| 5041 | gactgtctct ccaaatacca ccctagtcag ctccttggct tctcagtgtc cccttggaga |
| 5101 | acatggggta taggttccca gctagttcag tggcattcca cagcccatct cttgtgaggt |
| 5161 | cccactcctt aacaatggtc tttcagtttc aaacgcatgc ggccagcggg cagtctaggg |
| 5221 | acccttcaaa gtcaatgctt cttgattaaa ttatcgagac taatgtttaa ctttgagata |
| 5281 | cgttttctga gagttgctaa ccggttggag atgaacttag agaatagggt tcaccttttt |
| 5341 | cgtctgtcag cgggttatcg agtgcccagt ggtgtgccag attcagcagc tggtgcagga |
| 5401 | gatacattcg tgagcaaaac agatctgagc cctgacttcg ggaggcctcc tcctaacaac |
| 5461 | tagggcagat ataaccagtg ttccctgaat acaaacgcct agcctggcat ggtggcacac |
| 5521 | acctatgatg tcagccctta ggagccggag gtaagaagat caggagttca gctatctttg |
| 5581 | gccaacctgg gctacataag accgtgtctt aaaaaaaaaa aaaaaaatcc aaacaaaata |
| 5641 | cacactataa ctgtgagaaa tgttgtgaag agaaaggtcc aaatgcagtg aaagagctca |
| 5701 | gtaaaaaaag tgtggggtgt gttaggacag tgacaacatg tgcccgtatg tggagaagag |
| 5761 | aatcctgggt aatgggagga gcttactgta ttggaatcgg cagcagcagt gaggtctgct |
| 5821 | gctggacgga gcctgccccc caggctgggt ggggaaggtg tcacggacct tgcagaccac |
| 5881 | ggtaaggaac ttgcattctg gtgtttaact ttttatttgg agaccatttc aaagtgactg |
| 5941 | gaaccttatg agagtggcac aaaagatgtc tgcatacttt ggctgcagcc tccccgactg |
| 6001 | acctgtaaac gttctgttcc ccgagtcacc acccgtgtct ccctgtgatg tgtactcata |
| 6061 | gcctgtagtc cgaactctga gaatgagttg catacattgt gtctgtttac acttaaaaca |
| 6121 | cagtggagac cccctacagt aatgcctcgc ccgcctccgc ctgccacact gggtttatcg |
| 6181 | ctggttggtg gctccacact gtttgttggt cgtctctcta gtcaccttca ttagcatctt |
| 6241 | ccctttagga caagtcacgt ctgcgaatga tgtggaccat gcgttgtgct ttcttgctcg |
| 6301 | tatcttttaa tgtggcgtag tttctttcct ctctgtttga atagactatt tctccttttg |

SEQ ID NO: 169 Mouse BCL7C Amino Acid Sequence isoform 1 (NP_001334581.1)

| | |
|---|---|
| 1 | magrtvraet rsrakddikk vmatiekvrr wekrwvtvgd tslrifkwvp vvdpqeeerr |
| 61 | ragggaersr grerrgrgts prgggplill dindensnqs fhsegslqkg aepspggtpq |
| 121 | psrpgsptgp pevitedtqp pqlgqerdpg gtpaggtdep pkltkeepvp elleaedsgv |
| 181 | rltrralqek glkteplrrl lprrglrtns rptstvpepr apgsgskaqr aprtipqgkg |
| 241 | r |

SEQ ID NO: 170 Mouse BCL7C cDNA Sequence variant 2 (NM_009746.2; CDS:
240-893)

| | |
|---|---|
| 1 | ggccggggct ctagcagccc gcgccgcccg ggccgctccg gggacgggcc ggggcggggc |
| 61 | gcggtcttag gaagccaggc ggggacgcgc ggaggcgttg gggagcgagg gagggcgcgg |
| 121 | ccaactcccg gagggacggc aggccgaaag agcggcgctg gggcctggcg ctcagcctga |
| 181 | gatcgccgga ccacaggccg ccccgccacg ggctctgtcc cggccccagc cccgccagca |
| 241 | tggccggccg gaccgtgcgg gccgagaccc ggagccgggc caaagatgac atcaagaagg |
| 301 | tgatggcgac catcgagaag gtccggagat gggagaagcg ctgggtgact gtgggagaca |
| 361 | cttcccttcg aatcttcaag tgggtgcctg tggtggatcc ccaggaggag gagaggcggc |
| 421 | gggcaggagg cggggcagag agatcccgtg gccgggagag acgtggtagg ggcaccagtc |
| 481 | ccagaggggg aggccccctc atcctactgg atctcaatga tgagaacagc aaccagagtt |
| 541 | tccattctga aggttcattg caaaagggtg ctgagcccag ccctgggggg acgccccagc |
| 601 | ccagccgccc tggatcacca actggacccc cagaagtgat tactgaagat actcagcccc |
| 661 | cacaattggg tcaggagaga gatccagggg ggacacctgc aggcggtact gatgaacccc |
| 721 | caaagctgac caaggaggag cctgttccag aattgctaga agctgaggcc cccgaagctt |
| 781 | accctgtctt tgagccagtg ccatctgtcc ctgaggcagc ccagggtgac acagaggact |
| 841 | cggagggcgc ccccccactc aagcgcatct gtccaaatgc ccctgacccc tgagaagccg |
| 901 | cctgcctcct gtcctgttgc tccaggggcc cctttggctt tttataaata aagacccttt |
| 961 | tgtaaaaaaa aaaaaaaaaa a |

SEQ ID NO: 171 Mouse BCL7C Amino Acid Sequence isoform 2 (NP_033876.1)

| | |
|---|---|
| 1 | magrtvraet rsrakddikk vmatiekvrr wekrwvtvgd tslrifkwvp vvdpqeeerr |
| 61 | ragggaersr grerrgrgts prgggplill dindensnqs fhsegslqkg aepspggtpq |
| 121 | psrpgsptgp pevitedtqp pqlgqerdpg gtpaggtdep pkltkeepvp elleaeapea |
| 181 | ypvfepvpsv peaaqgdted segapplkri cpnapdp |

SEQ ID NO: 172 Human SMARCA2 Amino Acid Sequence Isoform A
(NP_001276325.1 and NP_003061.3)

| | |
|---|---|
| 1 | mstptdpgam phpgpspgpg pspgpilgps pgpgpspgsv hsmmgpspgp psvshpmptm |
| 61 | gstdfpqegm hqmhkpidgi hdkgivedih cgsmkgtgmr pphpgmgppq spmdqhsqgy |
| 121 | msphpsplga pehvsspmsg ggptppqmpp sqpgalipgd pqamsqpnrg pspfspvqlh |
| 181 | qlraqilayk mlargqplpe tlqlavqgkr sSpglqqqqq qqqqqqqqqq qqqqqqqqpq |
| 241 | qqppqpqtqq qqqpalvnyn rpsgpgpels gpstpqklpv papggrpspa ppaaaqppaa |
| 301 | avpgpsvpqp apgqpspvlq lqqkqsrisp iqkpqgldpv eilqereyrl qariahriqe |
| 361 | lenlpgslpp dlrtkatvel kalrllnfqr qlrqevvacm rrdttletal nskaykrskr |
| 421 | qtlrearmte klekqqkieq erkrrqkhqe ylnsilqhak dfkeyhrsva gkiqklskav |
| 481 | atwhantere qkketeriek ermrrlmaed eegyrklidq kkdrrlayll qqtdeyvanl |
| 541 | tnlvwehkqa qaakekkkrr rrkkkaeena eggesalgpd gepidessqm sdlpvkvtht |
| 601 | etgkvlfgpe apkasqldaw lemnpgyeva prsdseesds dyeeedeeee ssrqeteeki |
| 661 | lldpnseevs ekdakqiiet akqdvddeys mqysargsqs yytvahaise rvekqsalli |
| 721 | ngtlkhyqlq glewmvslyn nnlngilade mglgktiqti alitylmehk ringpyliiv |
| 781 | plstlsnwty efdkwapsvv kisykgtpam rrslvpqlrs gkfnvlltty eyiikdkhil |
| 841 | akirwkymiv deghrmknhh ckltqvlnth yvaprrillt gtplqnklpe lwallnfllp |
| 901 | tifkscstfe qwfnapfamt gervdlneee tiliirrlhk vlrpfllrrl kkevesqlpe |
| 961 | kveyvikcdm salqkilyrh mqakgilltd gsekdkkgkg gaktlmntim qlrkicnhpy |
| 1021 | mfqhieesfa ehlgysngvi ngaelyrasg kfelldrilp klratnhrvl lfcqmtslmt |
| 1081 | imedyfafrn flylrldgtt ksedraallk kfnepgsqyf ifllstragg 1glnlqaadt |
| 1141 | vvifdsdwnp hqdlqaqdra hrigqqnevr vlrlctvnsv eekilaaaky klnvdqkviq |
| 1201 | agmfdqksss herraflqai leheeeneee devpddetln qmiarreeef dlfmrmdmdr |
| 1261 | rredarnpkr kprlmeedel pswiikddae verltceeee ekifgrgsrq rrdvdysdal |
| 1321 | tekqwlraie dgnleemeee vrlkkrkrrr nvdkdpaked vekakkrrgr ppaeklspnp |
| 1381 | pkltkqmnai idtvinykdr cnvekvpsns qleiegnssg rqlsevfiql psrkelpeyy |
| 1441 | elirkpvdfk kikerirnhk yrslgdlekd vmllchnaqt fnlegsqiye dsivlqsvfk |
| 1501 | sarqkiakee esedesneee eeedeeeses eaksvkvkik Inkkddkgrd kgkgkkrpnr |
| 1561 | gkakpvvsdf dsdeeqdere qsegsgtdde |

SEQ ID NO: 173 Human SMARCA2 cDNA Sequence Variant 1 (NM_003070.4,
CDS: 223-4995)

| | |
|---|---|
| 1 | gcgtcttccg gcgcccgcgg aggaggcgag ggtgggacgc tgggcggagc ccgagtttag |
| 61 | gaagaggagg ggacggctgt catcaatgaa gtcatattca taatctagtc ctctctccct |
| 121 | ctgtttctgt actctgggtg actcagagag ggaagagatt cagccagcac actcctcgcg |
| 181 | agcaagcatt actctactga ctggcagaga caggagaggt agatgtccac gcccacagac |
| 241 | cctggtgcga tgccccaccc agggccttcg ccggggcctg ggccttcccc tgggccaatt |
| 301 | cttgggccta gtccaggacc aggaccatcc ccaggttccg tccacagcat gatggggcca |
| 361 | agtcctggac ctccaagtgt ctcccatcct atgccgacga tggggtccac agacttccca |
| 421 | caggaaggca tgcatcaaat gcataagccc atcgatggta tacatgacaa ggggattgta |
| 481 | gaagacatcc attgtggatc catgaagggc actggtatgc gaccacctca cccaggcatg |
| 541 | ggccctcccc agagtccaat ggatcaacac agccaaggtt atatgtcacc acacccatct |
| 601 | ccattaggag ccccagagca cgtctccagc cctatgtctg gaggaggccc aactccacct |
| 661 | cagatgccac caagccagcc gggggccctc atcccaggtg atccgcaggc catgagccag |
| 721 | cccaacagag gtccctcacc tttcagtcct gtccagctgc atcagcttcg agctcagatt |
| 781 | ttagcttata aaatgctggc ccgaggccag cccctccccg aaacgctgca gcttgcagtc |
| 841 | caggggaaaa ggacgttgcc tggcttgcag caacaacagc agcagcaaca gcagcagcag |
| 901 | cagcagcagc agcagcagca gcagcagcaa cagcagccgc agcagcagcc gccgcaacca |
| 961 | cagacgcagc aacaacagca gccggccctt gttaactaca acagaccatc tggcccgggg |
| 1021 | ccggagctga gcggcccgag caccccgcag aagctgccgg tgcccgcgcc cggcggccgg |
| 1081 | ccctcgcccg cgccccccgc agccgcgcag ccgcccgcgg ccgcagtgcc cgggccctca |
| 1141 | gtgccgcagc cggccccggg gcagccctcg cccgtcctcc agctgcagca gaagcagagc |
| 1201 | cgcatcagcc ccatccagaa accgcaaggc ctggaccccg tggaaattct gcaagagcgg |
| 1261 | gaatacagac ttcaggcccg catagctcat aggatacaag aactggaaaa tctgcctggc |
| 1321 | tctttgccac cagatttaag aaccaaagca accgtggaac taaaagcact tcggttactc |
| 1381 | aatttccagc gtcagctgag acaggaggtg gtggcctgca tgcgcaggga cacgaccctg |
| 1441 | gagacggctc tcaactccaa agcatacaaa cggagcaagc gccagactct gagagaagct |
| 1501 | cgcatgaccg agaagctgga gaagcagcag aagattgagc aggagaggaa acgccgtcag |
| 1561 | aaacaccagg aatacctgaa cagtattttg caacatgcaa aagattttaa ggaatatcat |
| 1621 | cggtctgtgg ccggaaagat ccagaagctc tccaaagcag tggcaacttg gcatgccaac |
| 1681 | actgaaagag agcagaagaa ggagacagag cggattgaaa aggagagaat gcggcgactg |
| 1741 | atggctgaag atgaggaggg ttatagaaaa ctgattgatc aaaagaaaga caggcgttta |
| 1801 | gcttaccttt tgcagcagac cgatgagtat gtagccaatc tgaccaatct ggtttgggag |
| 1861 | cacaagcaag cccaggcagc caaagagaag aagaagagga ggaggaggaa gaagaaggct |
| 1921 | gaggagaatg cagagggtgg ggagtctgcc ctgggaccgg atggagagcc catagatgag |
| 1981 | agcagccaga tgagtgacct ccctgtcaaa gtgactcaca cagaaaccgg caaggttctg |
| 2041 | ttcggaccag aagcacccaa agcaagtcag ctggacgcct ggctggaaat gaatcctggt |
| 2101 | tatgaagttg cccctagatc tgacagtgaa gagagtgatt ctgattatga ggaagaggat |
| 2161 | gaggaagaag agtccagtag gcaggaaacc gaagagaaaa tactcctgga tccaaatagc |
| 2221 | gaagaagttt ctgagaagga tgctaagcag atcattgaga cagctaagca agacgtggat |
| 2281 | gatgaataca gcatgcagta cagtgccagg ggctcccagt cctactacac cgtggctcat |
| 2341 | gccatctcgg agagggtgga gaaacagtct gccctcctaa ttaatgggac cctaaagcat |
| 2401 | taccagctcc agggcctgga atggatggtt tccctgtata ataacaactt gaacggaatc |
| 2461 | ttagccgatg aaatggggct tggaaagacc atacagacca ttgcactcat cacttatctg |
| 2521 | atggagcaca aaagactcaa tggcccctat ctcatcattg ttcccctttc gactctatct |
| 2581 | aactggacat atgaatttga caaatgggct ccttctgtgg tgaagatttc ttacaagggt |
| 2641 | actcctgcca tgcgtcgctc ccttgtcccc cagctacgga gtggcaaatt caatgtcctc |
| 2701 | ttgactactt atgagtatat tataaaagac aagcacattc ttgcaaagat tcggtggaaa |
| 2761 | tacatgatag tggacgaagg ccaccgaatg aagaatcacc actgcaagct gactcaggtc |
| 2821 | ttgaacactc actatgtggc ccccagaagg atcctcttga ctgggacccc gctgcagaat |
| 2881 | aagctccctg aactctgggc cctcctcaac ttcctcctcc caacaatttt taagagctgc |
| 2941 | agcacatttg aacaatggtt caatgctcca tttgccatga ctggtgaaag ggtggactta |
| 3001 | aatgaagaag aaactatatt gatcatcagg cgtctacata aggtgttaag accattttta |
| 3061 | ctaaggagac tgaagaaaga agttgaatcc cagcttcccg aaaaagtgga atatgtgatc |
| 3121 | aagtgtgaca tgtcagctct gcagaagatt ctgtatcgcc atatgcaagc caaggggatc |
| 3181 | cttctcacag atggttctga gaaagataag aaggggaaag gaggtgctaa gacacttatg |
| 3241 | aacactatta tgcagttgag aaaaatctgc aaccacccat atatgtttca gcacattgag |
| 3301 | gaatcctttg ctgaacacct aggctattca aatggggtca tcaatggggc tgaactgtat |
| 3361 | cgggcctcag ggaagtttga gctgcttgat cgtattctgc caaaattgag agcgactaat |
| 3421 | caccgagtgc tgcttttctg ccagatgaca tctctcatga ccatcatgga ggattatttt |
| 3481 | gcttttcgga acttccttta cctacgcctt gatggcacca ccaagtctga agatcgtgct |
| 3541 | gctttgctga agaaattcaa tgaacctgga tcccagtatt tcattttctt gctgagcaca |
| 3601 | agagctggtg gcctgggctt aaatcttcag gcagctgata cagtggtcat ctttgacagc |
| 3661 | gactggaatc ctcatcagga tctgcaggcc caagaccgag ctcaccgcat cgggcagcag |
| 3721 | aacgaggtcc gggtactgag gctctgtacc gtgaacagcg tggaggaaaa gatcctcgcg |
| 3781 | gccgcaaaat acaagctgaa cgtggatcag aaagtgatcc aggcgggcat gtttgaccaa |
| 3841 | aagtcttcaa gccacgagcg gagggcattc ctgcaggcca tcttggagca tgaggaggaa |
| 3901 | aatgaggaag aagatgaagt accggacgat gagactctga accaaatgat tgctcgacga |
| 3961 | gaagaagaat ttgacctttt tatgcggatg gacatggacc ggcggaggga agatgcccgg |
| 4021 | aacccgaaac ggaagccccg tttaatggag gaggatgagc tgccctcctg gatcattaag |
| 4081 | gatgacgctg aagtagaaag gctcacctgt gaagaagagg aggagaaaat atttgggagg |
| 4141 | gggtcccgcc agcgccgtga cgtggactac agtgacgccc tcacggagaa gcagtggcta |
| 4201 | agggccatcg aagacggcaa tttggaggaa atggaagagg aagtacggct taagaagcga |
| 4261 | aaaagacgaa gaaatgtgga taaagatcct gcaaaagaag atgtggaaaa agctaagaag |
| 4321 | agaagaggcc gccctcccgc tgagaaactg tcaccaaatc cccccaaact gacaaagcag |
| 4381 | atgaacgcta tcatcgatac tgtgataaac tacaaagata ggtgtaacgt ggagaaggtg |
| 4441 | cccagtaatt ctcagttgga aatagaagga aacagttcag ggcgacagct cagtgaagtc |
| 4501 | ttcattcagt taccttcaag gaaagaatta ccagaatact atgaattaat taggaagcca |
| 4561 | gtggatttca aaaaaataaa ggaaaggatt cgtaatcata agtaccggag cctaggcgac |
| 4621 | ctggagaagg atgtcatgct tctctgtcac aacgctcaga cgttcaacct ggagggatcc |
| 4681 | cagatctatg aagactccat cgtcttacag tcagtgttta agagtgcccg gcagaaaatt |
| 4741 | gccaaagagg aagagagtga ggatgaaagc aatgaagagg aggaagagga agatgaagaa |
| 4801 | gagtcagagt ccgaggcaaa atcagtcaag gtgaaaatta agctcaataa aaaagatgac |
| 4861 | aaaggccggg acaaagggaa aggcaagaaa aggccaaatc gaggaaaagc caaacctgta |
| 4921 | gtgagcgatt ttgacagcga tgaggagcag gatgaacgtg aacagtcaga aggaagtggg |
| 4981 | acggatgatg agtgatcagt atggaccttt ttccttggta gaactgaatt ccttcctccc |
| 5041 | ctgtctcatt tctacccagt gagttcattt gtcatatagg cactgggttg tttctatatc |
| 5101 | atcatcgtct ataaactagc tttaggatag tgccagacaa acatatgata tcatggtgta |
| 5161 | aaaaacacac acatacacaa atatttgtaa catattgtga ccaaatgggc ctcaaagatt |
| 5221 | cagattgaaa caaacaaaaa gcttttgatg gaaaatatgt gggtggatag tatatttcta |
| 5281 | tgggtgggtc taatttggta acggtttgat tgtgcctggt tttatcacct gttcagatga |
| 5341 | gaagattttt gtcttttgta gcactgataa ccaggagaag ccattaaaag ccactggtta |
| 5401 | ttttattttt catcaggcaa ttttcgaggt ttttatttgt tcggtattgt ttttttacac |
| 5461 | tgtggtacat ataagcaact ttaataggtg ataaatgtac agtagttaga tttcacctgc |
| 5521 | atatacattt ttccatttta tgctctatga tctgaacaaa agctttttga attgtataag |
| 5581 | atttatgtct actgtaaaca ttgcttaatt tttttgctct tgatttaaaa aaaagttttg |
| 5641 | ttgaaagcgc tattgaatat tgcaatctat atagtgtatt ggatggcttc ttttgtcacc |
| 5701 | ctgatctcct atgttaccaa tgtgtatcgt ctccttctcc ctaaagtgta cttaatcttt |
| 5761 | gctttctttg cacaatgtct ttggttgcaa gtcataagcc tgaggcaaat aaaattccag |
| 5821 | taatttcgaa gaatgtggtg ttggtgcttt cctaataaag aaataattta gcttgacaaa |
| 5881 | aaaaaaaaaa aa |

SEQ ID NO: 174 Human SMARCA2 cDNA Sequence Variant 3 (NM_001289396.1,
CDS: 210-4982)

| | |
|---|---|
| 1 | tcagaagaaa gccccgagat cacagagacc cggcgagatc acagagaccc ggcctgaagg |
| 61 | aacgtggaaa gaccaatgta cctgttttga ccggttgcct ggagcaagaa gttccagttg |
| 121 | gggagaattt tcagaagata aagtcggaga ttgtggaaag acttgacttg cagcattact |
| 181 | ctactgactg gcagagacag gagaggtaga tgtccacgcc cacagaccct ggtgcgatgc |
| 241 | cccacccagg gccttcgccg gggcctgggc cttcccctgg gccaattctt gggcctagtc |
| 301 | caggaccagg accatcccca ggttccgtcc acagcatgat ggggccaagt cctggacctc |
| 361 | caagtgtctc ccatcctatg ccgacgatgg ggtccacaga cttcccacag gaaggcatgc |
| 421 | atcaaatgca taagcccatc gatggtatac atgacaaggg gattgtagaa gacatccatt |
| 481 | gtggatccat gaagggcact ggtatgcgac cacctcaccc aggcatgggc cctccccaga |
| 541 | gtccaatgga tcaacacagc caaggttata tgtcaccaca cccatctcca ttaggagccc |
| 601 | cagagcacgt ctccagccct atgtctggag gaggcccaac tccacctcag atgccaccaa |
| 661 | gccagccggg ggccctcatc ccaggtgatc cgcaggccat gagccagccc aacagaggtc |
| 721 | cctcaccttt cagtcctgtc cagctgcatc agcttcgagc tcagatttta gcttataaaa |
| 781 | tgctggcccg aggccagccc ctccccgaaa cgctgcagct tgcagtccag gggaaaagga |
| 841 | cgttgcctgg cttgcagcaa caacagcagc agcaacagca gcagcagcag cagcagcagc |
| 901 | agcagcagca gcagcaacag cagccgcagc agcagccgcc gcaaccacag acgcagcaac |
| 961 | aacagcagcc ggcccttgtt aactacaaca gaccatctgg cccggggccg gagctgagcg |
| 1021 | gcccgagcac cccgcagaag ctgccggtgc ccgcgcccgg cggccggccc tcgcccgcgc |
| 1081 | cccccgcagc cgcgcagccg cccgcggccg cagtgcccgg gccctcagtg ccgcagccgg |
| 1141 | ccccggggca gccctcgccc gtcctccagc tgcagcagaa gcagagccgc atcagcccca |
| 1201 | tccagaaacc gcaaggcctg gaccccgtgg aaattctgca agagcgggaa tacagacttc |
| 1261 | aggcccgcat agctcatagg atacaagaac tggaaaatct gcctggctct ttgccaccag |
| 1321 | atttaagaac caaagcaacc gtggaactaa aagcacttcg gttactcaat ttccagcgtc |
| 1381 | agctgagaca ggaggtggtg gcctgcatgc gcagggacac gaccctggag acggctctca |
| 1441 | actccaaagc atacaaacgg agcaagcgcc agactctgag agaagctcgc atgaccgaga |
| 1501 | agctggagaa gcagcagaag attgagcagg agaggaaacg ccgtcagaaa caccaggaat |
| 1561 | acctgaacag tattttgcaa catgcaaaag attttaagga atatcatcgg tctgtggccg |
| 1621 | gaaagatcca gaagctctcc aaagcagtgg caacttggca tgccaacact gaaagagagc |
| 1681 | agaagaagga gacagagcgg attgaaaagg agagaatgcg gcgactgatg gctgaagatg |
| 1741 | aggagggtta tagaaaactg attgatcaaa agaaagacag gcgtttagct taccttttgc |
| 1801 | agcagaccga tgagtatgta gccaatctga ccaatctggt ttgggagcac aagcaagccc |
| 1861 | aggcagccaa agagaagaag aagaggagga ggaggaagaa gaaggctgag gagaatgcag |
| 1921 | agggtgggga gtctgccctg ggaccggatg gagagcccat agatgagagc agccagatga |
| 1981 | gtgacctccc tgtcaaagtg actcacacag aaaccggcaa ggttctgttc ggaccagaag |
| 2041 | cacccaaagc aagtcagctg gacgcctggc tggaaatgaa tcctggttat gaagttgccc |
| 2101 | ctagatctga cagtgaagag agtgattctg attatgagga agaggatgag gaagaagagt |
| 2161 | ccagtaggca ggaaaccgaa gagaaaatac tcctggatcc aaatagcgaa gaagtttctg |
| 2221 | agaaggatgc taagcagatc attgagacag ctaagcaaga cgtggatgat gaatacagca |
| 2281 | tgcagtacag tgccaggggc tcccagtcct actacaccgt ggctcatgcc atctcggaga |
| 2341 | gggtggagaa acagtctgcc ctcctaatta atgggaccct aaagcattac cagctccagg |
| 2401 | gcctggaatg gatggtttcc ctgtataata acaacttgaa cggaatctta gccgatgaaa |
| 2461 | tggggcttgg aaagaccata cagaccattg cactcatcac ttatctgatg gagcacaaaa |
| 2521 | gactcaatgg cccctatctc atcattgttc ccctttcgac tctatctaac tggacatatg |
| 2581 | aatttgacaa atgggctcct tctgtggtga agatttctta caagggtact cctgccatgc |
| 2641 | gtcgctccct tgtcccccag ctacggagtg gcaaattcaa tgtcctcttg actacttatg |
| 2701 | agtatattat aaaagacaag cacattcttg caaagattcg gtggaaatac atgatagtgg |
| 2761 | acgaaggcca ccgaatgaag aatcaccact gcaagctgac tcaggtcttg aacactcact |
| 2821 | atgtggcccc cagaaggatc ctcttgactg ggaccccgct gcagaataag ctccctgaac |
| 2881 | tctgggccct cctcaacttc ctcctcccaa caatttttaa gagctgcagc acatttgaac |
| 2941 | aatggttcaa tgctccattt gccatgactg gtgaaagggt ggacttaaat gaagaagaaa |
| 3001 | ctatattgat catcaggcgt ctacataagg tgttaagacc atttttacta aggagactga |
| 3061 | agaaagaagt tgaatcccag cttcccgaaa aagtggaata tgtgatcaag tgtgacatgt |
| 3121 | cagctctgca gaagattctg tatcgccata tgcaagccaa ggggatcctt ctcacagatg |
| 3181 | gttctgagaa agataagaag gggaaaggag gtgctaagac acttatgaac actattatgc |
| 3241 | agttgagaaa aatctgcaac cacccatata tgtttcagca cattgaggaa tcctttgctg |
| 3301 | aacacctagg ctattcaaat ggggtcatca atggggctga actgtatcgg gcctcaggga |
| 3361 | agtttgagct gcttgatcgt attctgccaa aattgagagc gactaatcac cgagtgctgc |
| 3421 | ttttctgcca gatgacatct ctcatgacca tcatggagga ttattttgct tttcggaact |
| 3481 | tcctttacct acgccttgat ggcaccacca agtctgaaga tcgtgctgct ttgctgaaga |
| 3541 | aattcaatga acctggatcc cagtatttca ttttcttgct gagcacaaga gctggtggcc |
| 3601 | tgggcttaaa tcttcaggca gctgatacag tggtcatctt tgacagcgac tggaatcctc |
| 3661 | atcaggatct gcaggcccaa gaccgagctc accgcatcgg gcagcagaac gaggtccggg |
| 3721 | tactgaggct ctgtaccgtg aacagcgtgg aggaaaagat cctcgcggcc gcaaaataca |
| 3781 | agctgaacgt ggatcagaaa gtgatccagg cgggcatgtt tgaccaaaag tcttcaagcc |
| 3841 | acgagcggag ggcattcctg caggccatct tggagcatga ggaggaaaat gaggaagaag |
| 3901 | atgaagtacc ggacgatgag actctgaacc aaatgattgc tcgacgagaa gaagaatttg |
| 3961 | acctttttat gcggatggac atggaccggc ggagggaaga tgcccggaac ccgaaacgga |
| 4021 | agccccgttt aatggaggag gatgagctgc cctcctggat cattaaggat gacgctgaag |
| 4081 | tagaaaggct cacctgtgaa gaagaggagg agaaaatatt tgggaggggg tcccgccagc |
| 4141 | gccgtgacgt ggactacagt gacgccctca cggagaagca gtggctaagg gccatcgaag |
| 4201 | acggcaattt ggaggaaatg gaagaggaag tacggcttaa gaagcgaaaa agacgaagaa |
| 4261 | atgtggataa agatcctgca aaagaagatg tggaaaaagc taagaagaga agaggccgcc |
| 4321 | ctcccgctga gaaactgtca ccaaatcccc ccaaactgac aaagcagatg aacgctatca |
| 4381 | tcgatactgt gataaactac aaagataggt gtaacgtgga gaaggtgccc agtaattctc |
| 4441 | agttggaaat agaaggaaac agttcagggc gacagctcag tgaagtcttc attcagttac |
| 4501 | cttcaaggaa agaattacca gaatactatg aattaattag gaagccagtg gatttcaaaa |
| 4561 | aaataaagga aaggattcgt aatcataagt accggagcct aggcgacctg gagaaggatg |
| 4621 | tcatgcttct ctgtcacaac gctcagacgt tcaacctgga gggatcccag atctatgaag |
| 4681 | actccatcgt cttacagtca gtgtttaaga gtgcccggca gaaaattgcc aaagaggaag |
| 4741 | agagtgagga tgaaagcaat gaagaggagg aagaggaaga tgaagaagag tcagagtccg |
| 4801 | aggcaaaatc agtcaaggtg aaaattaagc tcaataaaaa agatgacaaa ggccgggaca |
| 4861 | aagggaaagg caagaaaagg ccaaatcgag gaaaagccaa acctgtagtg agcgattttg |
| 4921 | acagcgatga ggagcaggat gaacgtgaac agtcagaagg aagtgggacg gatgatgagt |
| 4981 | gatcagtatg gacctttttc cttggtagaa ctgaattcct tcctcccctg tctcatttct |
| 5041 | acccagtgag ttcatttgtc atataggcac tgggttgttt ctatatcatc atcgtctata |
| 5101 | aactagcttt aggatagtgc cagacaaaca tatgatatca tggtgtaaaa aacacacaca |
| 5161 | tacacaaata tttgtaacat attgtgacca aatgggcctc aaagattcag attgaaacaa |
| 5221 | acaaaaagct tttgatggaa aatatgtggg tggatagtat atttctatgg gtgggtctaa |
| 5281 | tttggtaacg gtttgattgt gcctggtttt atcacctgtt cagatgagaa gatttttgtc |
| 5341 | ttttgtagca ctgataacca ggagaagcca ttaaaagcca ctggttattt tatttttcat |
| 5401 | caggcaattt tcgaggtttt tatttgttcg gtattgtttt tttacactgt ggtacatata |
| 5461 | agcaacttta ataggtgata aatgtacagt agttagattt cacctgcata tacatttttc |
| 5521 | cattttatgc tctatgatct gaacaaaagc tttttgaatt gtataagatt tatgtctact |
| 5581 | gtaaacattg cttaattttt ttgctcttga tttaaaaaaa agttttgttg aaagcgctat |
| 5641 | tgaatattgc aatctatata gtgtattgga tggcttcttt tgtcaccctg atctcctatg |
| 5701 | ttaccaatgt gtatcgtctc cttctcccta aagtgtactt aatctttgct ttctttgcac |
| 5761 | aatgtctttg gttgcaagtc ataagcctga ggcaaataaa attccagtaa tttcgaagaa |
| 5821 | tgtggtgttg gtgctttcct aataaagaaa taatttagct tgacaaaaaa aaaaaaaaa |

SEQ ID NO: 175 Human SMARCA2 Amino Acid Sequence Isoform B
(NP_620614.2)

| | |
|---|---|
| 1 | mstptdpgam phpgpspgpg pspgpilgps pgpgpspgsv hsmmgpspgp psvshpmptm |
| 61 | gstdfpqegm hqmhkpidgi hdkgivedih cgsmkgtgmr pphpgmgppq spmdqhsqgy |
| 121 | msphpsplga pehvsspmsg ggptppqmpp sqpgalipgd pqamsqpnrg pspfspvqlh |
| 181 | qlraqilayk mlargqplpe tlqlavqgkr tlpqlqqqqq qqqqqqqqqq qqqqqqqqpq |
| 241 | qqppqpqtqq qqqpalvnyn rpsgpgpels gpstpqklpv papggrpspa ppaaaqppaa |
| 301 | avpgpsvpqp apgqpspvlq lqqkqsrisp iqkpqgldpv eilqereyrl qariahriqe |
| 361 | lenlpgslpp dlrtkatvel kalrllnfqr qlrqevvacm rrdttletal nskaykrskr |
| 421 | qtlrearmte klekqqkieq erkrrqkhqe ylnsilqhak dfkeyhrsva gkiqklskav |
| 481 | atwhantere qkketeriek ermrrlmaed eegyrklidq kkdrrlayll qqtdeyvanl |
| 541 | tnlvwehkqa qaakekkkrr rrkkkaeena eggesalgpd gepidessqm sdlpvkvtht |
| 601 | etgkvlfgpe apkasqldaw lemnpgyeva prsdseesds dyeeedeeee ssrqeteeki |
| 661 | lldpnseevs ekdakqiiet akqdvddeys mqysargsqs yytvahaise rvekqsalli |
| 721 | ngtlkhyqlq glewmvslyn nnlngilade mglgktiqti alitylmehk ringpyliiv |
| 781 | plstlsnwty efdkwapsvv kisykgtpam rrslvpqlrs gkfnvlltty eyiikdkhil |
| 841 | akirwkymiv deghrmknhh ckltqvlnth yvaprrillt gtplqnklpe lwallnfllp |
| 901 | tifkscstfe qwfnapfamt gervdlneee tiliirrlhk vlrpfllrrl kkevesqlpe |
| 961 | kveyvikcdm salqkilyrh mqakgilltd gsekdkkgkg gaktlmntim qlrkicnhpy |
| 1021 | mfqhieesfa ehlgysngvi ngaelyrasg kfelldrilp klratnhrvl lfcqmtslmt |
| 1081 | imedyfafrn flylrldgtt ksedraallk kfnepgsqyf ifllstragg 1glnlqaadt |
| 1141 | vvifdsdwnp hqdlqaqdra hrigqqnevr vlrlctvnsv eekilaaaky klnvdqkviq |
| 1201 | agmfdqksss herraflqai leheeeneee devpddetln qmiarreeef dlfmrmdmdr |
| 1261 | rredarnpkr kprlmeedel pswiikddae verltceeee ekifgrgsrq rrdvdysdal |
| 1321 | tekqwlraie dgnleemeee vrlkkrkrrr nvdkdpaked vekakkrrgr ppaeklspnp |
| 1381 | pkltkqmnai idtvinykds sgrqlsevfi qlpsrkelpe yyelirkpvd fkkikerirn |
| 1441 | hkyrslgdle kdvmllchna qtfnlegsqi yedsivlqsv fksarqkiak eeesedesne |
| 1501 | eeeeedeees eseaksvkvk iklnkkddkg rdkgkgkkrp nrgkakpvvs dfdsdeeqde |
| 1561 | reqsegsgtd de |

SEQ ID NO: 176 Human SMARCA2 cDNA Sequence Variant 2 (NM_139045.3,
CDS: 223-4941)

| | |
|---|---|
| 1 | gcgtcttccg gcgcccgcgg aggaggcgag ggtgggacgc tgggcggagc ccgagtttag |
| 61 | gaagaggagg ggacggctgt catcaatgaa gtcatattca taatctagtc ctctctccct |
| 121 | ctgtttctgt actctgggtg actcagagag ggaagagatt cagccagcac actcctcgcg |
| 181 | agcaagcatt actctactga ctggcagaga caggagaggt agatgtccac gcccacagac |
| 241 | cctggtgcga tgccccaccc agggccttcg ccggggcctg ggccttcccc tgggccaatt |
| 301 | cttgggccta gtccaggacc aggaccatcc ccaggttccg tccacagcat gatggggcca |
| 361 | agtcctggac ctccaagtgt ctcccatcct atgccgacga tggggtccac agacttccca |
| 421 | caggaaggca tgcatcaaat gcataagccc atcgatggta tacatgacaa ggggattgta |
| 481 | gaagacatcc attgtggatc catgaagggc actggtatgc gaccacctca cccaggcatg |
| 541 | ggccctcccc agagtccaat ggatcaacac agccaaggtt atatgtcacc acacccatct |
| 601 | ccattaggag ccccagagca cgtctccagc cctatgtctg gaggaggccc aactccacct |
| 661 | cagatgccac caagccagcc gggggccctc atcccaggtg atccgcaggc catgagccag |
| 721 | cccaacagag gtccctcacc tttcagtcct gtccagctgc atcagcttcg agctcagatt |
| 781 | ttagcttata aaatgctggc ccgaggccag cccctccccg aaacgctgca gcttgcagtc |
| 841 | caggggaaaa ggacgttgcc tggcttgcag caacaacagc agcagcaaca gcagcagcag |
| 901 | cagcagcagc agcagcagca gcagcagcaa cagcagccgc agcagcagcc gccgcaacca |
| 961 | cagacgcagc aacaacagca gccggccctt gttaactaca acagaccatc tggcccgggg |
| 1021 | ccggagctga gcggcccgag caccccgcag aagctgccgg tgcccgcgcc cggcggccgg |
| 1081 | ccctcgcccg cgccccccgc agccgcgcag ccgcccgcgg ccgcagtgcc cgggccctca |
| 1141 | gtgccgcagc cggccccggg gcagccctcg cccgtcctcc agctgcagca gaagcagagc |
| 1201 | cgcatcagcc ccatccagaa accgcaaggc ctggaccccg tggaaattct gcaagagcgg |
| 1261 | gaatacagac ttcaggcccg catagctcat aggatacaag aactggaaaa tctgcctggc |
| 1321 | tctttgccac cagatttaag aaccaaagca accgtggaac taaaagcact tcggttactc |
| 1381 | aatttccagc gtcagctgag acaggaggtg gtggcctgca tgcgcaggga cacgaccctg |
| 1441 | gagacggctc tcaactccaa agcatacaaa cggagcaagc gccagactct gagagaagct |
| 1501 | cgcatgaccg agaagctgga gaagcagcag aagattgagc aggagaggaa acgccgtcag |
| 1561 | aaacaccagg aatacctgaa cagtattttg caacatgcaa aagattttaa ggaatatcat |
| 1621 | cggtctgtgg ccggaaagat ccagaagctc tccaaagcag tggcaacttg gcatgccaac |
| 1681 | actgaaagag agcagaagaa ggagacagag cggattgaaa aggagagaat gcggcgactg |
| 1741 | atggctgaag atgaggaggg ttatagaaaa ctgattgatc aaaagaaaga caggcgttta |
| 1801 | gcttaccttt tgcagcagac cgatgagtat gtagccaatc tgaccaatct ggtttgggag |
| 1861 | cacaagcaag cccaggcagc caaagagaag aagaagagga ggaggaggaa gaagaaggct |
| 1921 | gaggagaatg cagagggtgg ggagtctgcc ctgggaccgg atggagagcc catagatgag |
| 1981 | agcagccaga tgagtgacct ccctgtcaaa gtgactcaca cagaaaccgg caaggttctg |
| 2041 | ttcggaccag aagcacccaa agcaagtcag ctggacgcct ggctggaaat gaatcctggt |
| 2101 | tatgaagttg cccctagatc tgacagtgaa gagagtgatt ctgattatga ggaagaggat |
| 2161 | gaggaagaag agtccagtag gcaggaaacc gaagagaaaa tactcctgga tccaaatagc |
| 2221 | gaagaagttt ctgagaagga tgctaagcag atcattgaga cagctaagca agacgtggat |
| 2281 | gatgaataca gcatgcagta cagtgccagg ggctcccagt cctactacac cgtggctcat |
| 2341 | gccatctcgg agagggtgga gaaacagtct gccctcctaa ttaatgggac cctaaagcat |
| 2401 | taccagctcc agggcctgga atggatggtt tccctgtata ataacaactt gaacggaatc |
| 2461 | ttagccgatg aaatggggct tggaaagacc atacagacca ttgcactcat cacttatctg |
| 2521 | atggagcaca aaagactcaa tggcccctat ctcatcattg ttcccctttc gactctatct |
| 2581 | aactggacat atgaatttga caaatgggct ccttctgtgg tgaagatttc ttacaagggt |
| 2641 | actcctgcca tgcgtcgctc ccttgtcccc cagctacgga gtggcaaatt caatgtcctc |
| 2701 | ttgactactt atgagtatat tataaaagac aagcacattc ttgcaaagat tcggtggaaa |
| 2761 | tacatgatag tggacgaagg ccaccgaatg aagaatcacc actgcaagct gactcaggtc |
| 2821 | ttgaacactc actatgtggc ccccagaagg atcctcttga ctgggacccc gctgcagaat |
| 2881 | aagctccctg aactctgggc cctcctcaac ttcctcctcc caacaatttt taagagctgc |
| 2941 | agcacatttg aacaatggtt caatgctcca tttgccatga ctggtgaaag ggtggactta |
| 3001 | aatgaagaag aaactatatt gatcatcagg cgtctacata aggtgttaag accattttta |
| 3061 | ctaaggagac tgaagaaaga agttgaatcc cagcttcccg aaaaagtgga atatgtgatc |
| 3121 | aagtgtgaca tgtcagctct gcagaagatt ctgtatcgcc atatgcaagc caaggggatc |
| 3181 | cttctcacag atggttctga gaaagataag aaggggaaag gaggtgctaa gacacttatg |
| 3241 | aacactatta tgcagttgag aaaaatctgc aaccacccat atatgtttca gcacattgag |
| 3301 | gaatcctttg ctgaacacct aggctattca aatggggtca tcaatggggc tgaactgtat |
| 3361 | cgggcctcag ggaagtttga gctgcttgat cgtattctgc caaaattgag agcgactaat |
| 3421 | caccgagtgc tgcttttctg ccagatgaca tctctcatga ccatcatgga ggattatttt |
| 3481 | gcttttcgga acttccttta cctacgcctt gatggcacca ccaagtctga agatcgtgct |
| 3541 | gctttgctga agaaattcaa tgaacctgga tcccagtatt tcattttctt gctgagcaca |
| 3601 | agagctggtg gcctgggctt aaatcttcag gcagctgata cagtggtcat ctttgacagc |
| 3661 | gactggaatc ctcatcagga tctgcaggcc caagaccgag ctcaccgcat cgggcagcag |
| 3721 | aacgaggtcc gggtactgag gctctgtacc gtgaacagcg tggaggaaaa gatcctcgcg |
| 3781 | gccgcaaaat acaagctgaa cgtggatcag aaagtgatcc aggcgggcat gtttgaccaa |
| 3841 | aagtcttcaa gccacgagcg gagggcattc ctgcaggcca tcttggagca tgaggaggaa |
| 3901 | aatgaggaag aagatgaagt accggacgat gagactctga accaaatgat tgctcgacga |
| 3961 | gaagaagaat ttgacctttt tatgcggatg gacatggacc ggcggaggga agatgcccgg |
| 4021 | aacccgaaac ggaagccccg tttaatggag gaggatgagc tgccctcctg gatcattaag |
| 4081 | gatgacgctg aagtagaaag gctcacctgt gaagaagagg aggagaaaat atttgggagg |
| 4141 | gggtcccgcc agcgccgtga cgtggactac agtgacgccc tcacggagaa gcagtggcta |
| 4201 | agggccatcg aagacggcaa tttggaggaa atggaagagg aagtacggct taagaagcga |
| 4261 | aaaagacgaa gaaatgtgga taaagatcct gcaaaagaag atgtggaaaa agctaagaag |
| 4321 | agaagaggcc gccctcccgc tgagaaactg tcaccaaatc cccccaaact gacaaagcag |
| 4381 | atgaacgcta tcatcgatac tgtgataaac tacaaagata gttcagggcg acagctcagt |
| 4441 | gaagtcttca ttcagttacc ttcaaggaaa gaattaccag aatactatga attaattagg |
| 4501 | aagccagtgg atttcaaaaa aataaaggaa aggattcgta atcataagta ccggagccta |
| 4561 | ggcgacctgg agaaggatgt catgcttctc tgtcacaacg ctcagacgtt caacctggag |
| 4621 | ggatcccaga tctatgaaga ctccatcgtc ttacagtcag tgtttaagag tgcccggcag |
| 4681 | aaaattgcca aagaggaaga gagtgaggat gaaagcaatg aagaggagga agaggaagat |
| 4741 | gaagaagagt cagagtccga ggcaaaatca gtcaaggtga aaattaagct caataaaaaa |
| 4801 | gatgacaaag gccgggacaa agggaaaggc aagaaaaggc caaatcgagg aaaagccaaa |
| 4861 | cctgtagtga gcgattttga cagcgatgag gagcaggatg aacgtgaaca gtcagaagga |
| 4921 | agtgggacgg atgatgagtg atcagtatgg acctttttcc ttggtagaac tgaattcctt |
| 4981 | cctcccctgt ctcatttcta cccagtgagt tcatttgtca tataggcact gggttgtttc |
| 5041 | tatatcatca tcgtctataa actagcttta ggatagtgcc agacaaacat atgatatcat |
| 5101 | ggtgtaaaaa acacacacat acacaaatat ttgtaacata ttgtgaccaa atgggcctca |
| 5161 | aagattcaga ttgaaacaaa caaaaagctt ttgatggaaa atatgtgggt ggatagtata |
| 5221 | tttctatggg tgggtctaat ttggtaacgg tttgattgtg cctggtttta tcacctgttc |
| 5281 | agatgagaag atttttgtct tttgtagcac tgataaccag gagaagccat taaaagccac |
| 5341 | tggttatttt atttttcatc aggcaatttt cgaggttttt atttgttcgg tattgttttt |
| 5401 | ttacactgtg gtacatataa gcaactttaa taggtgataa atgtacagta gttagatttc |
| 5461 | acctgcatat acatttttcc attttatgct ctatgatctg aacaaaagct ttttgaattg |
| 5521 | tataagattt atgtctactg taaacattgc ttaatttttt tgctcttgat ttaaaaaaaa |
| 5581 | gttttgttga aagcgctatt gaatattgca atctatatag tgtattggat ggcttctttt |
| 5641 | gtcaccctga tctcctatgt taccaatgtg tatcgtctcc ttctccctaa agtgtactta |
| 5701 | atctttgctt tctttgcaca atgtctttgg ttgcaagtca taagcctgag gcaaataaaa |
| 5761 | ttccagtaat ttcgaagaat gtggtgttgg tgctttccta ataaagaaat aatttagctt |
| 5821 | gacaaaaaaa aaaaaaaa |

SEQ ID NO: 177 Human SMARCA2 Amino Acid Sequence Isoform C
(NP_001276326.1)

| | |
|---|---|
| 1 | mstptdpgam phpgpspgpg pspgpilgps pgpgpspgsv hsmmgpspgp psvshpmptm |
| 61 | gstdfpqegm hqmhkpidgi hdkgivedih cgsmkgtgmr pphpgmgppq spmdqhsqgy |
| 121 | msphpsplga pehvsspmsg ggptppqmpp sqpgalipgd pqamsqpnrg pspfspvqlh |
| 181 | qlraqilayk mlargqplpe tlglavqgkr tlpglqqqqq qqqqqqqqqq qqqqqqqqpq |
| 241 | qqppqpqtqq qqqpalvnyn rpsgpgpels gpstpqklpv papggrpspa ppaaaqppaa |
| 301 | avpgpsvpqp apgqpspvlq lqqkqsrisp iqkpqgldpv eilqereyrl qariahriqe |
| 361 | lenlpgslpp dlrtkatvel kalrllnfqr qlrqevvacm rrdttletal nskaykrskr |
| 421 | qtlrearmte klekqqkieq erkrrqkhqe ylnsilqhak dfkeyhrsva gkiqklskav |
| 481 | atwhantere qkketeriek ermrrlmaed eegyrklidq kkdrrlayll qqtdeyvanl |
| 541 | tnlvwehkqa qaakekkkrr rrkkkaeena eggesalgpd gepidessqm sdlpvkvtht |
| 601 | etgkvlfgpe apkasqldaw lemnpgyeva prsdseesds dyeeedeeee ssrqeteeki |
| 661 | lldpnseevs ekdakqiiet akqdvddeys mqysargsqs yytvahaise rvekqsalli |
| 721 | ngtlkhyqlq glewmvslyn nnlngilade mglgktiqti alitylmehk rlngpyliiv |
| 781 | plstlsnwty efdkwapsvv kisykgtpam rrslvpqlrs gkfnvlltty eyiikdkhil |
| 841 | akirwkymiv deghrmknhh ckltqvdlne eetiliirrl hkvlrpfllr rlkkevesql |
| 901 | pekveyvikc dmsalqkily rhmqakgill tdgsekdkkg kggaktlmnt imqlrkicnh |
| 961 | pymfqhiees faehlgysng vingaelyra sgkfelldri lpklratnhr vllfcqmtsl |
| 1021 | mtimedyfaf rnflylrldg ttksedraal lkkfnepgsq yfifllstra gglglnlqaa |
| 1081 | dtvvifdsdw nphqdlqaqd rahrigqqne vrvlrlctvn sveekilaaa kyklnvdqkv |
| 1141 | iqagmfdqks ssherraflq aileheeene eedevpddet lnqmiarree efdlfmrmdm |
| 1201 | drrredarnp krkprlmeed elpswiikdd aeverltcee eeekifgrgs rqrrdvdysd |
| 1261 | altekqwlra iedgnleeme eevrlkkrkr rrnvdkdpak edvekakkrr grppaeklsp |
| 1321 | nppkltkqmn aiidtvinyk dssgrqlsev fiqlpsrkel peyyelirkp vdfkkikeri |
| 1381 | rnhkyrslgd lekdvmilch naqtfnlegs qiyedsivlq svfksarqki akeeesedes |
| 1441 | neeeeeedee eseseaksvk vkiklnkkdd kgrdkgkgkk rpnrgkakpv vsdfdsdeeq |
| 1501 | dereqsegsg tdde |

SEQ ID NO: 178 Human SMARCA2 cDNA Sequence Variant 4 (NM_001289397.1,
CDS: 223-4767)

| | |
|---|---|
| 1 | gcgtcttccg gcgcccgcgg aggaggcgag ggtgggacgc tgggcggagc ccgagtttag |
| 61 | gaagaggagg ggacggctgt catcaatgaa gtcatattca taatctagtc ctctctccct |
| 121 | ctgtttctgt actctgggtg actcagagag ggaagagatt cagccagcac actcctcgcg |
| 181 | agcaagcatt actctactga ctggcagaga caggagaggt agatgtccac gcccacagac |
| 241 | cctggtgcga tgccccaccc agggccttcg ccggggcctg ggccttcccc tgggccaatt |
| 301 | cttgggccta gtccaggacc aggaccatcc ccaggttccg tccacagcat gatggggcca |
| 361 | agtcctggac ctccaagtgt ctcccatcct atgccgacga tggggtccac agacttccca |
| 421 | caggaaggca tgcatcaaat gcataagccc atcgatggta tacatgacaa ggggattgta |
| 481 | gaagacatcc attgtggatc catgaagggc actggtatgc gaccacctca cccaggcatg |
| 541 | ggccctcccc agagtccaat ggatcaacac agccaaggtt atatgtcacc acacccatct |
| 601 | ccattaggag ccccagagca cgtctccagc cctatgtctg gaggaggccc aactccacct |
| 661 | cagatgccac caagccagcc gggggccctc atcccaggtg atccgcaggc catgagccag |
| 721 | cccaacagag gtccctcacc tttcagtcct gtccagctgc atcagcttcg agctcagatt |
| 781 | ttagcttata aaatgctggc ccgaggccag cccctccccg aaacgctgca gcttgcagtc |
| 841 | caggggaaaa ggacgttgcc tggcttgcag caacaacagc agcagcaaca gcagcagcag |
| 901 | cagcagcagc agcagcagca gcagcagcaa cagcagccgc agcagcagcc gccgcaacca |
| 961 | cagacgcagc aacaacagca gccggccctt gttaactaca acagaccatc tggcccgggg |
| 1021 | ccggagctga gcggcccgag caccccgcag aagctgccgg tgcccgcgcc cggcggccgg |
| 1081 | ccctcgcccg cgccccccgc agccgcgcag ccgcccgcgg ccgcagtgcc cgggccctca |
| 1141 | gtgccgcagc cggccccggg gcagccctcg cccgtcctcc agctgcagca gaagcagagc |
| 1201 | cgcatcagcc ccatccagaa accgcaaggc ctggaccccg tggaaattct gcaagagcgg |
| 1261 | gaatacagac ttcaggcccg catagctcat aggatacaag aactggaaaa tctgcctggc |
| 1321 | tctttgccac cagatttaag aaccaaagca accgtggaac taaaagcact tcggttactc |
| 1381 | aatttccagc gtcagctgag acaggaggtg gtggcctgca tgcgcaggga cacgaccctg |
| 1441 | gagacggctc tcaactccaa agcatacaaa cggagcaagc gccagactct gagagaagct |
| 1501 | cgcatgaccg agaagctgga gaagcagcag aagattgagc aggagaggaa acgccgtcag |
| 1561 | aaacaccagg aatacctgaa cagtattttg caacatgcaa aagattttaa ggaatatcat |
| 1621 | cggtctgtgg ccggaaagat ccagaagctc tccaaagcag tggcaacttg gcatgccaac |
| 1681 | actgaaagag agcagaagaa ggagacagag cggattgaaa aggagagaat gcggcgactg |
| 1741 | atggctgaag atgaggaggg ttatagaaaa ctgattgatc aaaagaaaga caggcgttta |
| 1801 | gcttaccttt tgcagcagac cgatgagtat gtagccaatc tgaccaatct ggtttgggag |
| 1861 | cacaagcaag cccaggcagc caaagagaag aagaagagga ggaggaggaa gaagaaggct |
| 1921 | gaggagaatg cagagggtgg ggagtctgcc ctgggaccgg atggagagcc catagatgag |
| 1981 | agcagccaga tgagtgacct ccctgtcaaa gtgactcaca cagaaaccgg caaggttctg |
| 2041 | ttcggaccag aagcacccaa agcaagtcag ctggacgcct ggctggaaat gaatcctggt |
| 2101 | tatgaagttg cccctagatc tgacagtgaa gagagtgatt ctgattatga ggaagaggat |
| 2161 | gaggaagaag agtccagtag gcaggaaacc gaagagaaaa tactcctgga tccaaatagc |
| 2221 | gaagaagttt ctgagaagga tgctaagcag atcattgaga cagctaagca agacgtggat |
| 2281 | gatgaataca gcatgcagta cagtgccagg ggctcccagt cctactacac cgtggctcat |
| 2341 | gccatctcgg agagggtgga gaaacagtct gccctcctaa ttaatgggac cctaaagcat |
| 2401 | taccagctcc agggcctgga atggatggtt tccctgtata ataacaactt gaacggaatc |
| 2461 | ttagccgatg aaatggggct tggaaagacc atacagacca ttgcactcat cacttatctg |
| 2521 | atggagcaca aaagactcaa tggcccctat ctcatcattg ttcccctttc gactctatct |
| 2581 | aactggacat atgaatttga caaatgggct ccttctgtgg tgaagatttc ttacaagggt |
| 2641 | actcctgcca tgcgtcgctc ccttgtcccc cagctacgga gtggcaaatt caatgtcctc |
| 2701 | ttgactactt atgagtatat tataaaagac aagcacattc ttgcaaagat tcggtggaaa |
| 2761 | tacatgatag tggacgaagg ccaccgaatg aagaatcacc actgcaagct gactcaggtg |
| 2821 | gacttaaatg aagaagaaac tatattgatc atcaggcgtc tacataaggt gttaagacca |
| 2881 | tttttactaa ggagactgaa gaaagaagtt gaatcccagc ttcccgaaaa agtggaatat |
| 2941 | gtgatcaagt gtgacatgtc agctctgcag aagattctgt atcgccatat gcaagccaag |
| 3001 | gggatccttc tcacagatgg ttctgagaaa gataagaagg ggaaaggagg tgctaagaca |
| 3061 | cttatgaaca ctattatgca gttgagaaaa atctgcaacc acccatatat gtttcagcac |
| 3121 | attgaggaat cctttgctga acacctaggc tattcaaatg gggtcatcaa tggggctgaa |
| 3181 | ctgtatcggg cctcagggaa gtttgagctg cttgatcgta ttctgccaaa attgagagcg |
| 3241 | actaatcacc gagtgctgct tttctgccag atgacatctc tcatgaccat catggaggat |
| 3301 | tattttgctt ttcggaactt cctttaccta cgccttgatg gcaccaccaa gtctgaagat |
| 3361 | cgtgctgctt tgctgaagaa attcaatgaa cctggatccc agtatttcat tttcttgctg |
| 3421 | agcacaagag ctggtggcct gggcttaaat cttcaggcag ctgatacagt ggtcatcttt |
| 3481 | gacagcgact ggaatcctca tcaggatctg caggcccaag accgagctca ccgcatcggg |
| 3541 | cagcagaacg aggtccgggt actgaggctc tgtaccgtga acagcgtgga ggaaaagatc |
| 3601 | ctcgcggccg caaaatacaa gctgaacgtg gatcagaaag tgatccaggc gggcatgttt |
| 3661 | gaccaaaagt cttcaagcca cgagcggagg gcattcctgc aggccatctt ggagcatgag |
| 3721 | gaggaaaatg aggaagaaga tgaagtaccg gacgatgaga ctctgaacca aatgattgct |
| 3781 | cgacgagaag aagaatttga cctttttatg cggatggaca tggaccggcg gagggaagat |
| 3841 | gcccggaacc cgaaacggaa gccccgttta atggaggagg atgagctgcc ctcctggatc |
| 3901 | attaaggatg acgctgaagt agaaaggctc acctgtgaag aagaggagga gaaaatattt |
| 3961 | gggagggggt cccgccagcg ccgtgacgtg gactacagtg acgccctcac ggagaagcag |
| 4021 | tggctaaggg ccatcgaaga cggcaatttg gaggaaatgg aagaggaagt acggcttaag |
| 4081 | aagcgaaaaa gacgaagaaa tgtggataaa gatcctgcaa aagaagatgt ggaaaaagct |
| 4141 | aagaagagaa gaggccgccc tcccgctgag aaactgtcac caaatccccc caaactgaca |
| 4201 | aagcagatga acgctatcat cgatactgtg ataaactaca aagatagttc agggcgacag |
| 4261 | ctcagtgaag tcttcattca gttaccttca aggaaagaat taccagaata ctatgaatta |
| 4321 | attaggaagc cagtggattt caaaaaaata aaggaaagga ttcgtaatca taagtaccgg |
| 4381 | agcctaggcg acctggagaa ggatgtcatg cttctctgtc acaacgctca gacgttcaac |
| 4441 | ctggagggat cccagatcta tgaagactcc atcgtcttac agtcagtgtt taagagtgcc |
| 4501 | cggcagaaaa ttgccaaaga ggaagagagt gaggatgaaa gcaatgaaga ggaggaagag |
| 4561 | gaagatgaag aagagtcaga gtccgaggca aaatcagtca aggtgaaaat taagctcaat |
| 4621 | aaaaaagatg acaaaggccg ggacaaaggg aaaggcaaga aaaggccaaa tcgaggaaaa |
| 4681 | gccaaacctg tagtgagcga ttttgacagc gatgaggagc aggatgaacg tgaacagtca |
| 4741 | gaaggaagtg ggacggatga tgagtgatca gtatggacct ttttccttgg tagaactgaa |
| 4801 | ttccttcctc ccctgtctca tttctaccca gtgagttcat ttgtcatata ggcactgggt |
| 4861 | tgtttctata tcatcatcgt ctataaacta gctttaggat agtgccagac aaacatatga |
| 4921 | tatcatggtg taaaaaacac acacatacac aaatatttgt aacatattgt gaccaaatgg |
| 4981 | gcctcaaaga ttcagattga aacaaacaaa aagcttttga tggaaaatat gtgggtggat |
| 5041 | agtatatttc tatgggtggg tctaatttgg taacggtttg attgtgcctg gttttatcac |
| 5101 | ctgttcagat gagaagattt ttgtcttttg tagcactgat aaccaggaga agccattaaa |
| 5161 | agccactggt tattttattt ttcatcaggc aattttcgag gtttttattt gttcggtatt |
| 5221 | gtttttttac actgtggtac atataagcaa ctttaatagg tgataaatgt acagtagtta |
| 5281 | gatttcacct gcatatacat ttttccattt tatgctctat gatctgaaca aaagcttttt |
| 5341 | gaattgtata agatttatgt ctactgtaaa cattgcttaa tttttttgct cttgatttaa |
| 5401 | aaaaaagttt tgttgaaagc gctattgaat attgcaatct atatagtgta ttggatggct |
| 5461 | tcttttgtca ccctgatctc ctatgttacc aatgtgtatc gtctccttct ccctaaagtg |
| 5521 | tacttaatct ttgctttctt tgcacaatgt ctttggttgc aagtcataag cctgaggcaa |
| 5581 | ataaaattcc agtaatttcg aagaatgtgg tgttggtgct ttcctaataa agaaataatt |
| 5641 | tagcttgaca aaaaaaaaaa aaaa |

SEQ ID NO: 179 Human SMARCA2 Amino Acid Sequence Isoform D
(NP_001276327.1)

| | |
|---|---|
| 1 | mwlaiedgnl eemeeevrlk krkrrrnvdk dpakedveka kkrrgrppae klspnppklt |
| 61 | kqmnaiidtv inykdssgrq lsevfiqlps rkelpeyyel irkpvdfkki kerirnhkyr |
| 121 | slgdlekdvm llchnaqtfn legsqiyeds ivlqsvfksa rqkiakeees edesneeeee |
| 181 | edeeesesea ksvkvkikln kkddkgrdkg kgkkrpnrgk akpvvsdfds deeqdereqs |
| 241 | egsgtdde |

SEQ ID NO: 180 Human SMARCA2 cDNA Sequence Variant 5 (NM_001289398.1,
CDS: 203-949)

| | |
|---|---|
| 1 | cttggagagg cggaggtgga aacgatgcgc aggagttggc ttggggcttt ttgtttgcgt |
| 61 | gtccctgttt acctattcat aatcatggat cccctctgct ttgtgatact gtgaaccacg |
| 121 | cataacagca attctttaca ccaccgggtt gagaagaagg cgcctgaggc tgactttctg |
| 181 | gacctgccgt cacgcagtaa agatgtggtt ggccatcgaa gacggcaatt tggaggaaat |
| 241 | ggaagaggaa gtacggctta agaagcgaaa aagacgaaga aatgtggata aagatcctgc |
| 301 | aaaagaagat gtggaaaaag ctaagaagag aagaggccgc cctcccgctg agaaactgtc |
| 361 | accaaatccc cccaaactga caaagcagat gaacgctatc atcgatactg tgataaacta |
| 421 | caaagatagt tcagggcgac agctcagtga agtcttcatt cagttacctt caaggaaaga |
| 481 | attaccagaa tactatgaat taattaggaa gccagtggat ttcaaaaaaa taaaggaaag |
| 541 | gattcgtaat cataagtacc ggagcctagg cgacctggag aaggatgtca tgcttctctg |
| 601 | tcacaacgct cagacgttca acctggaggg atcccagatc tatgaagact ccatcgtctt |
| 661 | acagtcagtg tttaagagtg cccggcagaa aattgccaaa gaggaagaga gtgaggatga |
| 721 | aagcaatgaa gaggaggaag aggaagatga agaagagtca gagtccgagg caaaatcagt |
| 781 | caaggtgaaa attaagctca ataaaaaaga tgacaaaggc cgggacaaag ggaaaggcaa |
| 841 | gaaaaggcca aatcgaggaa aagccaaacc tgtagtgagc gattttgaca gcgatgagga |
| 901 | gcaggatgaa cgtgaacagt cagaaggaag tgggacggat gatgagtgat cagtatggac |
| 961 | ctttttcctt ggtagaactg aattccttcc tcccctgtct catttctacc cagtgagttc |
| 1021 | atttgtcata taggcactgg gttgtttcta tatcatcatc gtctataaac tagctttagg |
| 1081 | atagtgccag acaaacatat gatatcatgg tgtaaaaaac acacacatac acaaatattt |
| 1141 | gtaacatatt gtgaccaaat gggcctcaaa gattcagatt gaaacaaaca aaaagctttt |
| 1201 | gatggaaaat atgtgggtgg atagtatatt tctatgggtg ggtctaattt ggtaacggtt |
| 1261 | tgattgtgcc tggttttatc acctgttcag atgagaagat ttttgtcttt tgtagcactg |
| 1321 | ataaccagga gaagccatta aaagccactg gttattttat ttttcatcag gcaattttcg |
| 1381 | aggtttttat ttgttcggta ttgttttttt acactgtggt acatataagc aactttaata |
| 1441 | ggtgataaat gtacagtagt tagatttcac ctgcatatac atttttccat tttatgctct |
| 1501 | atgatctgaa caaaagcttt ttgaattgta taagatttat gtctactgta aacattgctt |
| 1561 | aatttttttg ctcttgattt aaaaaaaagt tttgttgaaa gcgctattga atattgcaat |
| 1621 | ctatatagtg tattggatgg cttcttttgt caccctgatc tcctatgtta ccaatgtgta |
| 1681 | tcgtctcctt ctccctaaag tgtacttaat ctttgctttc tttgcacaat gtctttggtt |
| 1741 | gcaagtcata agcctgaggc aaataaaatt ccagtaattt cgaagaatgt ggtgttggtg |
| 1801 | ctttcctaat aaagaaataa tttagcttga caaaaaaaaa aaaaaa |

SEQ ID NO: 181 Human SMARCA2 Amino Acid Sequence Isoform E
(NP_001276328.1)

| | |
|---|---|
| 1 | mkrlaarcfa gllilspltv isdsrpadsg kaiedgnlee meeevrlkkr krrrnvdkdp |
| 61 | akedvekakk rrgrppaekl spnppkltkq mnaiidtvin ykdssgrqls evfiqlpsrk |
| 121 | elpeyyelir kpvdfkkike rirnhkyrsl gdlekdvmll chnaqtfnle gsqiyedsiv |
| 181 | lqsvfksarq kiakeeesed esneeeeeed eeeseseaks vkvkiklnkk ddkgrdkgkg |
| 241 | kkrpnrgkak pvvsdfdsde eqdereqseg sgtdde |

SEQ ID NO: 182 Human SMARCA2 cDNA Sequence Variant 6 (NM_001289399.1,
CDS: 106-936)

| | |
|---|---|
| 1 | attcacttca ttaaatctag aggcagttga gcatgggagc cgtctgtatg ttgaattagg |
| 61 | gctcgcactc ttgcgcaaca cgtcaccagt cggaaactgg ggctgatgaa gagactagca |
| 121 | gctcgctgct ttgctggctt gttaatttta tccccactaa ctgtgatttc tgatagccgg |
| 181 | cctgctgata gtggtaaggc catcgaagac ggcaatttgg aggaaatgga agaggaagta |
| 241 | cggcttaaga agcgaaaaag acgaagaaat gtggataaag atcctgcaaa agaagatgtg |
| 301 | gaaaaagcta agaagagaag aggccgccct cccgctgaga aactgtcacc aaatcccccc |
| 361 | aaactgacaa agcagatgaa cgctatcatc gatactgtga taaactacaa agatagttca |
| 421 | gggcgacagc tcagtgaagt cttcattcag ttaccttcaa ggaaagaatt accagaatac |
| 481 | tatgaattaa ttaggaagcc agtggatttc aaaaaaataa aggaaaggat tcgtaatcat |
| 541 | aagtaccgga gcctaggcga cctggagaag gatgtcatgc ttctctgtca caacgctcag |
| 601 | acgttcaacc tggagggatc ccagatctat gaagactcca tcgtcttaca gtcagtgttt |
| 661 | aagagtgccc ggcagaaaat tgccaaagag gaagagagtg aggatgaaag caatgaagag |
| 721 | gaggaagagg aagatgaaga agagtcagag tccgaggcaa aatcagtcaa ggtgaaaatt |
| 781 | aagctcaata aaaaagatga caaaggccgg gacaaaggga aaggcaagaa aaggccaaat |
| 841 | cgaggaaaag ccaaacctgt agtgagcgat tttgacagcg atgaggagca ggatgaacgt |
| 901 | gaacagtcag aaggaagtgg gacggatgat gagtgatcag tatggacctt tttccttggt |
| 961 | agaactgaat tccttcctcc cctgtctcat ttctacccag tgagttcatt tgtcatatag |
| 1021 | gcactgggtt gtttctatat catcatcgtc tataaactag ctttaggata gtgccagaca |
| 1081 | aacatatgat atcatggtgt aaaaaacaca cacatacaca aatatttgta acatattgtg |
| 1141 | accaaatggg cctcaaagat tcagattgaa acaaacaaaa agcttttgat ggaaaatatg |
| 1201 | tgggtggata gtatatttct atgggtgggt ctaatttggt aacggtttga ttgtgcctgg |
| 1261 | ttttatcacc tgttcagatg agaagatttt tgtcttttgt agcactgata accaggagaa |
| 1321 | gccattaaaa gccactggtt attttatttt tcatcaggca attttcgagg tttttatttg |
| 1381 | ttcggtattg tttttttaca ctgtggtaca tataagcaac tttaataggt gataaatgta |
| 1441 | cagtagttag atttcacctg catatacatt tttccatttt atgctctatg atctgaacaa |
| 1501 | aagctttttg aattgtataa gatttatgtc tactgtaaac attgcttaat ttttttgctc |
| 1561 | ttgatttaaa aaaaagtttt gttgaaagcg ctattgaata ttgcaatcta tatagtgtat |
| 1621 | tggatggctt cttttgtcac cctgatctcc tatgttacca atgtgtatcg tctccttctc |
| 1681 | cctaaagtgt acttaatctt tgctttcttt gcacaatgtc tttggttgca agtcataagc |
| 1741 | ctgaggcaaa taaaattcca gtaatttcga agaatgtggt gttggtgctt tcctaataaa |
| 1801 | gaaataattt agcttgacaa aaaaaaaaaa aaa |

SEQ ID NO: 183 Human SMARCA2 Amino Acid Sequence Isoform F
(NP_001276329.1)

| | |
|---|---|
| 1 | mlmkrlaarc fagllilspl tvisdsrpad sgkaiedgnl eemeeevilk krkrrrnvdk |
| 61 | dpakedveka kkrrgrppae klspnppklt kqmnaiidtv inykdssgrq lsevfiqlps |
| 121 | rkelpeyyel irkpvdfkki kerirnhkyr slgdlekdvm llchnaqtfn legsqiyeds |
| 181 | ivlqsvfksa rqkiakeees edesneeeee edeeesesea ksvkvkikln kkddkgrdkg |
| 241 | kgkkrpnrgk akpvvsdfds deeqdereqs egsgtdde |

SEQ ID NO: 184 Human SMARCA2 cDNA Sequence Variant 7 (NM_001289400.1,
CDS: 521-1357)

| | |
|---|---|
| 1 | acttcattaa atctagaggc agttgagcat gggagccgtc tgtatgttga attagggctc |
| 61 | gcactcttgc gcaacacgtc accagtcgga aactgggggt ttgcttctgt gatttatttc |
| 121 | attattgtgc tggtaaaagg tttggaaggg aattcttttt gggggtagta ctttagcatt |
| 181 | gtgtagcaag ttttggggtt ttttttgtgt gtgacccccc agcccccagc gctgagtttg |
| 241 | agtcagttga gccagtttag taaataattt tttaaaataa aagaacagtt taaaatctcc |
| 301 | atgaataatt ttacttacat gcaggagtaa tcttactcta ctctttatgt gcgaaaagca |
| 361 | ttgggaagtg tttagtgaat tgatttccat tagaaaaaga cccttagaaa tcacagaaca |
| 421 | taaagcactg catatggatg tgtttggggt ctttggggag gagggaagat gttttgtagc |
| 481 | tctctgcatt cctgcataaa accttagttt gaggggaata atgctgatga agagactagc |
| 541 | agctcgctgc tttgctggct tgttaatttt atccccacta actgtgattt ctgatagccg |
| 601 | gcctgctgat agtggtaagg ccatcgaaga cggcaatttg gaggaaatgg aagaggaagt |
| 661 | acggcttaag aagcgaaaaa gacgaagaaa tgtggataaa gatcctgcaa aagaagatgt |
| 721 | ggaaaaagct aagaagagaa gaggccgccc tcccgctgag aaactgtcac caaatccccc |
| 781 | caaactgaca aagcagatga acgctatcat cgatactgtg ataaactaca aagatagttc |
| 841 | agggcgacag ctcagtgaag tcttcattca gttaccttca aggaaagaat taccagaata |
| 901 | ctatgaatta attaggaagc cagtggattt caaaaaaata aaggaaagga ttcgtaatca |
| 961 | taagtaccgg agcctaggcg acctggagaa ggatgtcatg cttctctgtc acaacgctca |
| 1021 | gacgttcaac ctggagggat cccagatcta tgaagactcc atcgtcttac agtcagtgtt |
| 1081 | taagagtgcc cggcagaaaa ttgccaaaga ggaagagagt gaggatgaaa gcaatgaaga |
| 1141 | ggaggaagag gaagatgaag aagagtcaga gtccgaggca aaatcagtca aggtgaaaat |
| 1201 | taagctcaat aaaaaagatg acaaaggccg ggacaaaggg aaaggcaaga aaaggccaaa |
| 1261 | tcgaggaaaa gccaaacctg tagtgagcga ttttgacagc gatgaggagc aggatgaacg |
| 1321 | tgaacagtca gaaggaagtg ggacggatga tgagtgatca gtatggacct ttttccttgg |
| 1381 | tagaactgaa ttccttcctc ccctgtctca tttctaccca gtgagttcat ttgtcatata |
| 1441 | ggcactgggt tgtttctata tcatcatcgt ctataaacta gctttaggat agtgccagac |
| 1501 | aaacatatga tatcatggtg taaaaaacac acacatacac aaatatttgt aacatattgt |
| 1561 | gaccaaatgg gcctcaaaga ttcagattga aacaaacaaa aagcttttga tggaaaatat |
| 1621 | gtgggtggat agtatatttc tatgggtggg tctaatttgg taacggtttg attgtgcctg |
| 1681 | gttttatcac ctgttcagat gagaagattt ttgtcttttg tagcactgat aaccaggaga |
| 1741 | agccattaaa agccactggt tattttattt ttcatcaggc aattttcgag gtttttattt |
| 1801 | gttcggtatt gtttttttac actgtggtac atataagcaa ctttaatagg tgataaatgt |
| 1861 | acagtagtta gatttcacct gcatatacat ttttccattt tatgctctat gatctgaaca |
| 1921 | aaagcttttt gaattgtata agatttatgt ctactgtaaa cattgcttaa tttttttgct |
| 1981 | cttgatttaa aaaaaagttt tgttgaaagc gctattgaat attgcaatct atatagtgta |
| 2041 | ttggatggct tcttttgtca ccctgatctc ctatgttacc aatgtgtatc gtctccttct |
| 2101 | ccctaaagtg tacttaatct ttgctttctt tgcacaatgt ctttggttgc aagtcataag |
| 2161 | cctgaggcaa ataaaattcc agtaatttcg aagaatgtgg tgttggtgct ttcctaataa |
| 2221 | agaaataatt tagcttgaca aaaaaaaaaa aaaa |

SEQ ID NO: 185 Mouse SMARCA2 cDNA Sequence variant 1 (NM_011416.2; CDS:
111-4862)

| | |
|---|---|
| 1 | ctcgctccct ctgtttctgt actctgggtg actcagagag ggaagattca gccagcacac |
| 61 | tgctcgcgag caagtgtcac tctgctaact ggcagagcca ggagacctag atgtccacac |
| 121 | ccacagaccc agcagcaatg ccccatcctg ggccctcccc ggggcctgga ccctctcctg |
| 181 | gaccaattct ggggcctagt ccaggaccag gaccatcccc aggttctgtg cacagcatga |
| 241 | tgggtcctag tcccggacct cccagcgtct cacatcctct gtcaacgatg ggctctgcag |
| 301 | acttcccaca ggaaggcatg caccaattac ataagcccat ggatgggata catgacaaag |
| 361 | ggattgtaga agatgtccac tgtggatcca tgaagggcac cagcatgcgc cccccacacc |
| 421 | caggaatggg ccctccacag agccccatgg accagcacag ccaaggttat atgtcaccac |
| 481 | atccgtctcc tctgggagcc ccggagcacg tctctagccc tatatctgga ggaggcccaa |
| 541 | ccccacccca gatgccaccg agccagccag gggcactcat cccaggagat ccgcaggcca |
| 601 | tgaaccagcc taacagaggt ccctcgcctt tcagtcctgt gcagctgcat cagcttcgag |
| 661 | ctcagatttt agcttacaaa atgttggcca ggggccagcc tctccctgaa actctgcagc |
| 721 | tggcagtcca gggaaaaagg accttgcctg gcatgcagca gcagcagcag caacaacaac |
| 781 | aacagcagca gcagcagcag cagcagcagc agcaacagca gcaacaacag cagccccagc |
| 841 | agcctcagca gcaggctcag gcacagcccc agcagcagca gcaacagcag cagcagccag |
| 901 | ctcttgttag ctataatcga ccatctggcc ccgggcagga gctgctactg agtggccaga |
| 961 | gcgctccgca gaagctgtca gcaccagcac caagcggccg accttcaccg gcaccccagg |
| 1021 | ccgccgtcca gcccacggcc acagcggtgc ccgggccctc cgtgcagcag cccgccccag |
| 1081 | ggcagccgtc tccggtccta cagctgcaac agaagcagag ccgcatcagc cccatccaga |
| 1141 | aaccgcaagg cctggacccg gtggagatcc tgcaggaacg agagtacaga cttcaagctc |
| 1201 | gcatcgctca taggatacaa gaactggaaa gtctgcctgg ttccttgcca ccagatttac |
| 1261 | gcaccaaagc aaccgtggaa ctgaaagcac ttcgcttact caacttccaa cgtcagctga |
| 1321 | gacaggaggt ggtggcctgc atgcggaggg acaccaccct ggagacggcc ctcaactcca |
| 1381 | aagcatataa gcggagcaag cgccagaccc tgcgtgaggc acgcatgaca gagaaactgg |
| 1441 | agaagcagca gaagatagaa caggagagga aacgccggca gaaacaccag gaatacctga |
| 1501 | acagtatttt gcaacatgca aaagatttta aggaatatca ccggtctgtg gccgggaaga |
| 1561 | tccagaagct ctccaaagca gtggcgactt ggcatgctaa cacagaaagg gagcagaaga |
| 1621 | aggagacgga gcggatcgag aaggagagaa tgcggaggct gatggccgaa gatgaagagg |
| 1681 | gctacaggaa gcttattgac caaaagaaag acagacgtct cgcctaccta ttgcagcaga |
| 1741 | ccgatgagta tgtcgccaat ctgaccaacc tggtgtggga gcacaagcag gcccaagcag |
| 1801 | ccaaagagaa gaagaagagg aggaggagga agaagaaggc tgaagagaat gcagagggag |
| 1861 | gggaacctgc cctgggacca gatggagagc caatagatga aagcagccag atgagtgacc |
| 1921 | tgcctgtcaa agtgacacac acagaaactg gcaaggtcct ctttggacca gaagcaccca |
| 1981 | aagcaagtca gctggatgcc tggctggaga tgaatcctgg ttacgaagtt gcacccagat |
| 2041 | ctgacagtga agagagtgaa tcggactacg aggaggagga tgaagaagaa gagtccagta |
| 2101 | ggcaggaaac cgaggagaag atactgctgg atcccaacag tgaagaagtt tccgaaaagg |
| 2161 | atgccaagca gatcattgag actgcgaagc aggacgtgga cgacgaatac agcatgcagt |
| 2221 | acagtgccag aggctctcag tcctactaca cggtggctca cgctatctct gagagggtgg |
| 2281 | agaagcagtc tgccctcctc attaacggca ccctaaagca ttaccagctc cagggcctgg |
| 2341 | aatggatggt ttccctgtat aataacaatc tgaacggaat cttagctgat gaaatggggc |
| 2401 | taggcaagac catccagacc attgcactca tcacgtatct gatggagcac aaaaggctca |
| 2461 | atggtcccta cctcatcatc gtccccctct cgactctgtc taactggaca tatgaatttg |
| 2521 | acaaatgggc tccttctgtg gtgaaaattt cttacaaggg tacccctgcc atgcgacgct |
| 2581 | ccctcgttcc ccagctacgg agtggcaaat tcaatgtcct cctgactact tacgagtaca |
| 2641 | ttataaaaga caagcacatt cttgcaaaga ttcggtggaa gtacatgatc gtggacgaag |
| 2701 | gccaccggat gaagaatcac cactgcaagc taacccaggt cctgaacaca cactatgtgg |
| 2761 | cccccaggcg gatccttctg actgggaccc cactgcagaa taagcttccg gaactctggg |
| 2821 | ccctcctcaa cttcctcctc cctacaatct tcaagagttg cagcacattt gagcagtggt |
| 2881 | ttaatgctcc atttgccatg accggtgaaa gggtggacct gaacgaagaa gaaacgattt |
| 2941 | tgatcatcag gcgtctacac aaggtgctga gacccttttt actgaggagg ctgaagaaag |
| 3001 | aggttgagtc tcagcttccg gaaaaggttg agtatgtgat caagtgtgac atgtcagctc |
| 3061 | tgcagaagat tctgtaccgt cacatgcaag ccaaggggat cctcctcacg gacgggtctg |
| 3121 | agaaagataa gaaggggaaa ggaggtgcca agacacttat gaacaccatc atgcagctga |
| 3181 | gaaaaatatg caaccaccca tatatgtttc agcacattga ggaatccttt gctgaacacc |
| 3241 | tgggctattc gaatggggtc atcaatgggg ctgagctgta tcgggcctcg ggaaagtttg |
| 3301 | agctgcttga tcgtattctg cccaaattga gagcgactaa ccaccgcgtg ctgcttttct |
| 3361 | gccagatgac gtcactcatg accattatgg aggattactt tgcttttcgg aacttcctgt |
| 3421 | acctgcgcct tgacggcacc accaagtctg aagatcgtgc tgctttgcta aagaaattca |
| 3481 | atgaacctgg gtcccagtat ttcattttct tgctgagcac aagagcaggg ggcctgggct |
| 3541 | taaatcttca ggcggcagac acggtggtca tatttgacag cgactggaat cctcaccagg |
| 3601 | atctgcaggc ccaagaccga gctcaccgca ttggccaaca aaacgaggtc cgggtgctga |
| 3661 | ggctttgcac cgtcaacagt gtggaggaaa agattctcgc ggctgccaag tacaagctga |
| 3721 | acgtggatca gaaggttatc caagcaggca tgtttgacca gaagtcatcc agccacgagc |
| 3781 | ggagggcctt cctgcaggcc attctggagc acgaggagga gaatgaggaa gaagatgagg |
| 3841 | taccagacga cgagaccctg aaccagatga ttgctcgccg ggaggaagaa tttgatcttt |
| 3901 | ttatgcgcat ggacatggac cggcggaggg aggatgcccg gaacccgaag cgcaaacccc |
| 3961 | gcttgatgga ggaagatgag ctgccctcct ggattatcaa ggatgacgcc gaagtggaaa |
| 4021 | ggctcacctg tgaagaagag gaggagaaga tatttgggag gggctctcgc cagcgccggg |
| 4081 | atgtggacta cagtgatgcc ctcaccgaga agcaatggct cagggccatc gaagacggca |
| 4141 | atttggaaga aatggaagag gaggtacggc ttaagaagag aaaaagacga agaaatgtgg |
| 4201 | ataaagaccc cgtgaaggaa gatgtggaaa aagcgaagaa aagaagaggc cgccctccgg |
| 4261 | ctgagaagtt gtcaccaaat cccccaaaac taacgaagca gatgaacgcc atcattgata |
| 4321 | ctgtgataaa ctacaaagac agttcagggc gacagctcag tgaagtcttc attcagttac |
| 4381 | cttccaggaa agacttacca gaatactatg aattaattag gaagccagtg gatttcaaaa |
| 4441 | agataaagga gcgaatccgt aatcataagt atcggagcct gggagacctg gagaaagacg |
| 4501 | tcatgcttct ctgtcacaac gcacagacat tcaacttgga aggatcccag atctacgaag |
| 4561 | actccattgt cctacagtca gtgtttaaga gtgctcggca gaaaattgcc aaagaagaag |
| 4621 | agagtgagga agaaagcaat gaagaagagg aagaagatga tgaagaggag tcggagtcag |
| 4681 | aggcgaaatc tgtgaaggtg aaaatcaagc tgaataaaaa ggaagagaaa ggccgggaca |
| 4741 | cagggaaggg caagaagcgg ccaaaccgag gcaaagccaa acccgtcgtg agcgattttg |
| 4801 | acagtgacga ggaacaggaa gagaacgaac agtcagaagc aagtggaact gataacgagt |
| 4861 | gaccatcctg gacgtgagct tcccgcggtg gcagaaccga atgctttctt ccccctctcc |
| 4921 | ttcctcccca gtgagttcac ttgccattcg ggcacactgg gttatttctc cgtcctcatt |
| 4981 | gtcatctaga actagcttta gggtagtgcc agacaaacat atgatatcat ggtgtaaaaa |
| 5041 | aagaaacaca tgcgtgcaga cacactacac acacacacac acacacacac acacacacac |
| 5101 | acacatattt gtaacatatt gtgaccaaat gggcctcaaa gattcaaaga ttaaaaacaa |
| 5161 | aaagcttttg atggaaaaga tgtgggtgga tagtatattt ctacaggtgg gtcaggtttg |
| 5221 | gtagcagttt gatgtgctgg gttctgtcat ctgttctgat gagaagattt ttatcttctg |
| 5281 | cagtgctgat ggccgggagg aaccattcaa agccactggt tattttgttt ttcatcaggc |
| 5341 | gattttcaag attttcattt gtttcagtat tgttggtttt ctcttttctc ttttttacac |
| 5401 | tgtggtacat ataagcaact tgactagtga caaatgtaca gtagttagat atcacctaca |
| 5461 | tatacatttt tccattttat gctctatgat ctgaagaaca aaaaaaaaag ctttttgact |
| 5521 | tgtataagat ttatgtctac tgtaaacatt gcggaatttt tttttgttct tgttttattg |
| 5581 | acaatgctat tgagtattac agtgtctaga ataccctgga tggcttctct tgtccacccg |
| 5641 | atctcccgtg ttaccaatgt gtatggtctc cttctcccga aagtgtactt aatctttgct |
| 5701 | ttctttgcac aatgtctttg gttgcaagtc ataagcctga ggcaaataaa attccagtaa |
| 5761 | tttccaagaa tgtggtgttg gtactttcct aataaaccga taacgtacct tgaaaaaaaa |
| 5821 | aaaaaaaaaa a |

SEQ ID NO: 186 Mouse SMARCA2 Amino Acid Sequence isoform 1 (NP_035546.2)

| | |
|---|---|
| 1 | mstptdpaam phpgpspgpg pspgpilgps pgpgpspgsv hsmmgpspgp psvshplstm |
| 61 | gsadfpqegm hqlhkpmdgi hdkgivedvh cgsmkgtsmr pphpgmgppq spmdqhsqgy |
| 121 | msphpsplga pehvsspisg ggptppqmpp sqpgalipgd pqamnqpnrg pspfspvqlh |
| 241 | qpqqpqqqaq aqpqqqqqqq qqpalvsynr psgpgqelll sgqsapqkls apapsgrpsp |
| 301 | apqaavqpta tavpgpsvqq papgqpspvl qlqqkqsris piqkpqgldp veilqereyr |
| 361 | lqariahriq eleslpgslp pdlrtkatve lkalrllnfq rqlrqevvac mrrdttleta |
| 421 | lnskaykrsk rqtlrearmt eklekqqkie qerkrrqkhq eylnsilqha kdfkeyhrsv |
| 481 | agkiqklska vatwhanter eqkketerie kermrrlmae deegyrklid qkkdrrlayl |
| 541 | lqqtdeyvan ltnlvwehkq aqaakekkkr rrrkkkaeen aeggepalgp dgepidessq |
| 601 | msdlpvkvth tetgkvlfgp eapkasqlda wlemnpgyev aprsdseese sdyeeedeee |
| 661 | essrqeteek illdpnseev sekdakqiie takqdvddey smqysargsq syytvahais |
| 721 | ervekqsall ingtlkhyql qglewmvsly nnnlngilad emglgktiqt ialitylmeh |
| 781 | krlngpylii vplstlsnwt yefdkwapsv vkisykgtpa mrrslvpqlr sgkfnvlltt |
| 841 | yeyiikdkhi lakirwkymi vdeghrmknh hckltqvlnt hyvaprrill tgtplqnklp |
| 901 | elwallnfll ptifkscstf eqwfnapfam tgervdlnee etiliirrlh kvlrpfllrr |
| 961 | lkkevesqlp ekveyvikcd msalqkilyr hmqakgillt dgsekdkkgk ggaktlmnti |
| 1021 | mqlrkicnhp ymfqhieesf aehlgysngv ingaelyras gkfelldril pklratnhrv |
| 1081 | llfcqmtslm timedyfafr nflylrldgt tksedraall kkfnepgsqy fifllstrag |
| 1141 | glglnlqaad tvvifdsdwn phqdlqaqdr ahrigqqnev rvlrlctvns veekilaaak |
| 1201 | yklnvdqkvi qagmfdqkss sherraflqa ileheeenee edevpddetl nqmiarreee |
| 1261 | fdlfmrmdmd rrredarnpk rkprlmeede lpswiikdda everltceee eekifgrgsr |
| 1321 | qrrdvdysda ltekqwlrai edgnleemee evrlkkrkrr rnvdkdpvke dvekakkrrg |
| 1381 | rppaeklspn ppkltkqmna iidtvinykd ssgrqlsevf iqlpsrkdlp eyyelirkpv |
| 1441 | dfkkikerir nhkyrslgdl ekdvmllchn aqtfnlegsq iyedsivlqs vfksarqkia |
| 1501 | keeeseeesn eeeeeddeee seseaksvkv kiklnkkeek grdtgkgkkr pnrgkakpvv |
| 1561 | sdfdsdeeqe eneqseasgt dne |

SEQ ID NO: 187 Mouse SMARCA2 cDNA Sequence variant 2 (NM_026003.2; CDS:
301-1011)

| | |
|---|---|
| 1 | ttcacttcat taaatctaga ggcggttcag catgggagcc gtctgtatgt tgaattaggg |
| 61 | ctcgctctct tgcgcaacac gtcaccagtc ggaaactggg ggtttgcttc tgtgatttat |
| 121 | ttcattattg tgctggtaaa agctgatgaa gagactagca gctcgctgct ttgccggctt |
| 181 | gttaatttta tccccactaa ctgtgatttc cgatagccgg cctgctgata gtggtaagtg |
| 241 | cggctggctc tggtttaaag caagcgtttg caggccatcg aagacggcaa tttggaagaa |
| 301 | atggaagagg aggtacggct taagaagaga aaaagacgaa gaaatgtgga taaagacccc |
| 361 | gtgaaggaag atgtggaaaa agcgaagaaa agaagaggcc gccctccggc tgagaagttg |
| 421 | tcaccaaatc ccccaaaact aacgaagcag atgaacgcca tcattgatac tgtgataaac |
| 481 | tacaaagaca gttcagggcg acagctcagt gaagtcttca ttcagttacc ttccaggaaa |
| 541 | gacttaccag aatactatga attaattagg aagccagtgg atttcaaaaa gataaaggag |
| 601 | cgaatccgta atcataagta tcggagcctg ggagacctgg agaaagacgt catgcttctc |
| 661 | tgtcacaacg cacagacatt caacttggaa ggatcccaga tctacgaaga ctccattgtc |
| 721 | ctacagtcag tgtttaagag tgctcggcag aaaattgcca aagaagaaga gagtgaggaa |
| 781 | gaaagcaatg aagaagagga agaagatgat gaagaggagt cggagtcaga ggcgaaatct |
| 841 | gtgaaggtga aaatcaagct gaataaaaag gaagagaaag gccgggacac agggaagggc |
| 901 | aagaagcggc caaaccgagg caaagccaaa cccgtcgtga gcgattttga cagtgacgag |
| 961 | gaacaggaag agaacgaaca gtcagaagca agtggaactg ataacgagtg accatcctgg |
| 1021 | acgtgagctt cccgcggtgg cagaaccgaa tgctttcttc cccctctcct tcctccccag |
| 1081 | tgagttcact tgccattcgg gcacactggg ttatttctcc gtcctcattg tcatctagaa |
| 1141 | ctagctttag ggtagtgcca gacaaacata tgatatcatg gtgtaaaaaa agaaacacat |
| 1201 | gcgtgcagac acactacaca cacacacaca cacacacaca cacacacaca cacatatttg |
| 1261 | taacatattg tgaccaaatg ggcctcaaag attcaaagat taaaaacaaa aagcttttga |
| 1321 | tggaaaagat gtgggtggat agtatatttc tacaggtggg tcaggtttgg tagcagtttg |
| 1381 | atgtgctggg ttctgtcatc tgttctgatg agaagatttt tatcttctgc agtgctgatg |
| 1441 | gccgggagga accattcaaa gccactggtt attttgtttt tcatcaggcg attttcaaga |
| 1501 | ttttcatttg tttcagtatt gttggttttc tcttttctct tttttacact gtggtacata |
| 1561 | taagcaactt gactagtgac aaatgtacag tagttagata tcacctacat atacattttt |
| 1621 | ccattttatg ctctatgatc tgaagaacaa aaaaaaaagc tttttgactt gtataagatt |
| 1681 | tatgtctact gtaaacattg cggaattttt ttttgttctt gttttattga caatgctatt |
| 1741 | gagtattaca gtgtctagaa taccctggat ggcttctctt gtccacccga tctcccgtgt |
| 1801 | taccaatgtg tatggtctcc ttctcccgaa agtgtactta atctttgctt tctttgcaca |
| 1861 | atgtctttgg ttgcaagtca taagcctgag gcaaataaaa ttccagtaat ttccaagaat |
| 1921 | gtggtgttgg tactttccta ataaaccgat aacgtacctt gaaa |

SEQ ID NO: 188 Mouse SMARCA2 Amino Acid Sequence isoform 2 (NP_080279.1)

| | |
|---|---|
| 1 | meeevilkkr krrrnvdkdp vkedvekakk rrgrppaekl spnppkltkq mnaiidtvin |
| 61 | ykdssgrqls evfiqlpsrk dlpeyyelir kpvdfkkike rirnhkyrsl gdlekdvmll |
| 121 | chnaqtfnle gsqiyedsiv lqsvfksarq kiakeeesee esneeeeedd eeeseseaks |
| 181 | vkvkiklnkk eekgrdtgkg kkrpnrgkak pvvsdfdsde eqeeneqsea sgtdne |

SEQ ID NO: 189 Mouse SMARCA2 cDNA Sequence variant 3 (NM_001347439.1;
CDS: 180-1010)

| | |
|---|---|
| 1 | acacacacac acacacacac acgcaggctg aagtatgctt aactctttta acttggctgg |
| 61 | ggctttttag caccatatgg gttctttcgt gacgtccgga cccgaaagag tgcagtgtgc |
| 121 | ctttaaggaa agaggtacct caccaaactt ccctgtagtt gtgcctcacc atttagctga |
| 181 | tgaagagact agcagctcgc tgctttgccg gcttgttaat tttatcccca ctaactgtga |
| 241 | tttccgatag ccggcctgct gatagtggta aggccatcga agacggcaat ttggaagaaa |
| 301 | tggaagagga ggtacggctt aagaagagaa aaagacgaag aaatgtggat aaagaccccg |
| 361 | tgaaggaaga tgtggaaaaa gcgaagaaaa gaagaggccg ccctccggct gagaagttgt |
| 421 | caccaaatcc cccaaaacta acgaagcaga tgaacgccat cattgatact gtgataaact |
| 481 | acaaagacag ttcagggcga cagctcagtg aagtcttcat tcagttacct tccaggaaag |
| 541 | acttaccaga atactatgaa ttaattagga agccagtgga tttcaaaaag ataaaggagc |
| 601 | gaatccgtaa tcataagtat cggagcctgg gagacctgga gaaagacgtc atgcttctct |
| 661 | gtcacaacgc acagacattc aacttggaag gatcccagat ctacgaagac tccattgtcc |
| 721 | tacagtcagt gtttaagagt gctcggcaga aaattgccaa agaagaagag agtgaggaag |
| 781 | aaagcaatga agaagaggaa gaagatgatg aagaggagtc ggagtcagag gcgaaatctg |
| 841 | tgaaggtgaa aatcaagctg aataaaaagg aagagaaagg ccgggacaca gggaagggca |
| 901 | agaagcggcc aaaccgaggc aaagccaaac ccgtcgtgag cgattttgac agtgacgagg |
| 961 | aacaggaaga gaacgaacag tcagaagcaa gtggaactga taacgagtga ccatcctgga |
| 1021 | cgtgagcttc ccgcggtggc agaaccgaat gctttcttcc ccctctcctt cctccccagt |
| 1081 | gagttcactt gccattcggg cacactgggt tatttctccg tcctcattgt catctagaac |
| 1141 | tagctttagg gtagtgccag acaaacatat gatatcatgg tgtaaaaaaa gaaacacatg |
| 1201 | cgtgcagaca cactacacac acacacacac acacacacac acacacacac acatatttgt |
| 1261 | aacatattgt gaccaaatgg gcctcaaaga ttcaaagatt aaaaacaaaa agcttttgat |
| 1321 | ggaaaagatg tgggtggata gtatatttct acaggtgggt caggtttggt agcagtttga |
| 1381 | tgtgctgggt tctgtcatct gttctgatga gaagattttt atcttctgca gtgctgatgg |
| 1441 | ccgggaggaa ccattcaaag ccactggtta ttttgttttt catcaggcga ttttcaagat |
| 1501 | tttcatttgt ttcagtattg ttggttttct cttttctctt ttttacactg tggtacatat |
| 1561 | aagcaacttg actagtgaca aatgtacagt agttagatat cacctacata tacatttttc |
| 1621 | cattttatgc tctatgatct gaagaacaaa aaaaaaagct ttttgacttg tataagattt |
| 1681 | atgtctactg taaacattgc ggaatttttt tttgttcttg ttttattgac aatgctattg |
| 1741 | agtattacag tgtctagaat accctggatg gcttctcttg tccacccgat ctcccgtgtt |
| 1801 | accaatgtgt atggtctcct tctcccgaaa gtgtacttaa tctttgcttt ctttgcacaa |
| 1861 | tgtctttggt tgcaagtcat aagcctgagg caaataaaat tccagtaatt tccaagaatg |
| 1921 | tggtgttggt actttcctaa taaaccgata acgtaccttg aaaaaaaaaa aaaaaaaaa |

SEQ ID NO: 190 Mouse SMARCA2 Amino Acid Sequence isoform 3
(NP_001334368.1)

| | |
|---|---|
| 1 | mkrlaarcfa gllilspltv isdsrpadsg kaiedgnlee meeevrlkkr krrrnvdkdp |
| 61 | vkedvekakk rrgrppaekl spnppkltkq mnaiidtvin ykdssgrqls evfiqlpsrk |
| 121 | dlpeyyelir kpvdfkkike rirnhkyrsl gdlekdvmll chnaqtfnle gsqiyedsiv |
| 181 | lqsvfksarq kiakeeesee esneeeeedd eeeseseaks vkvkiklnkk eekgrdtgkg |
| 241 | kkrpnrgkak pvvsdfdsde eqeeneqsea sgtdne |

SEQ ID NO: 191 Human SMARCA4 Amino Acid Sequence Isoform A
(NP_001122321.1)

| | |
|---|---|
| 1 | mstpdpplgg tprpgpspgp gpspgamlgp spgpspgsah smmgpspgpp saghpiptqg |
| 61 | pggypqdnmh qmhkpmesmh ekgmsddpry nqmkgmgmrs gghagmgppp spmdqhsqgy |
| 121 | psplggseha sspvpasgps sgpqmssgpg gapldgadpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa pmvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfkeyh rsvtgkiqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkaggle awlemnpgye vaprsdsees |
| 661 | gseeeeeeee eeqpqaaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdesr |
| 1261 | hcstgsgsas fahtapppag vnpdleeppl keedevpdde tvnqmiarhe eefdlfmrmd |
| 1321 | ldrrreearn pkrkprlmee delpswiikd daeverltce eeeekmfgrg srhrkevdys |
| 1381 | dsltekqwlk kitgkdihdt assvarglqf qrglqfctra skaieegtle eieeevrqkk |
| 1441 | ssrkrkrdsd agsstpttst rsrdkddesk kqkkrgrppa eklspnppnl tkkmkkivda |
| 1501 | vikykdsssg rqlsevfiql psrkelpeyy elirkpvdfk kikerirnhk yrslndlekd |
| 1561 | vmllcqnaqt fnlegsliye dsivlqsvft svrqkieked dsegeeseee eegeeegses |
| 1621 | esrsvkvkik lgrkekaqdr 1kggrrrpsr gsrakpvvsd ddseeeqeed rsgsgseed |

SEQ ID NO: 192 Human SMARCA4 cDNA Sequence Variant 1 (NM_001128849.1,
CDS: 75-5114)

| | |
|---|---|
| 1 | ggcgggggag gcgccgggaa gtcgacggcg ccggcggctc ctgcaggagg ccactgtctg |
| 61 | cagctcccgt gaagatgtcc actccagacc cacccctggg cggaactcct cggccaggtc |
| 121 | cttccccggg ccctggccct tcccctggag ccatgctggg ccctagcccg ggtccctcgc |
| 181 | cgggctccgc ccacagcatg atggggccca gcccagggcc gccctcagca ggacacccca |
| 241 | tccccaccca ggggcctgga gggtaccctc aggacaacat gcaccagatg cacaagccca |
| 301 | tggagtccat gcatgagaag ggcatgtcgg acgacccgcg ctacaaccag atgaaaggaa |
| 361 | tggggatgcg gtcagggggc catgctggga tggggccccc gcccagcccc atggaccagc |
| 421 | actcccaagg ttacccctcg cccctgggtg gctctgagca tgcctctagt ccagttccag |
| 481 | ccagtggccc gtcttcgggg ccccagatgt cttccgggcc aggaggtgcc ccgctggatg |
| 541 | gtgctgaccc ccaggccttg gggcagcaga accggggccc aaccccattt aaccagaacc |
| 601 | agctgcacca gctcagagct cagatcatgg cctacaagat gctggccagg gggcagcccc |
| 661 | tccccgacca cctgcagatg gcggtgcagg gcaagcggcc gatgcccggg atgcagcagc |
| 721 | agatgccaac gctacctcca ccctcggtgt ccgcaacagg acccggccct ggccctggcc |
| 781 | ctggccccgg cccgggtccc ggcccggcac ctccaaatta cagcaggcct catggtatgg |
| 841 | gagggcccaa catgcctccc ccaggaccct cgggcgtgcc ccccgggatg ccaggccagc |
| 901 | ctcctggagg gcctcccaag ccctggcctg aaggacccat ggcgaatgct gctgccccca |
| 961 | cgagcacccc tcagaagctg attcccccgc agccaacggg ccgcccttcc cccgcgcccc |
| 1021 | ctgccgtccc acccgccgcc tcgcccgtga tgccaccgca gacccagtcc cccgggcagc |
| 1081 | cggcccagcc cgcgcccatg gtgccactgc accagaagca gagccgcatc acccccatcc |
| 1141 | agaagccgcg gggcctcgac cctgtggaga tcctgcagga gcgcgagtac aggctgcagg |
| 1201 | ctcgcatcgc acaccgaatt caggaacttg aaaaccttcc cgggtccctg gccggggatt |
| 1261 | tgcgaaccaa agcgaccatt gagctcaagg ccctcaggct gctgaacttc cagaggcagc |
| 1321 | tgcgccagga ggtggtggtg tgcatgcgga gggacacagc gctggagaca gccctcaatg |
| 1381 | ctaaggccta caagcgcagc aagcgccagt ccctgcgcga ggcccgcatc actgagaagc |
| 1441 | tggagaagca gcagaagatc gagcaggagc gcaagcgccg gcagaagcac caggaatacc |
| 1501 | tcaatagcat tctccagcat gccaaggatt tcaaggaata tcacagatcc gtcacaggca |
| 1561 | aaatccagaa gctgaccaag gcagtggcca cgtaccatgc caacacggag cgggagcaga |
| 1621 | agaaagagaa cgagcggatc gagaaggagc gcatgcggag gctcatggct gaagatgagg |
| 1681 | aggggtaccg caagctcatc gaccagaaga aggacaagcg cctggcctac ctcttgcagc |
| 1741 | agacagacga gtacgtggct aacctcacgg agctggtgcg gcagcacaag gctgcccagg |
| 1801 | tcgccaagga gaaaaagaag aaaaagaaaa agaagaaggc agaaaatgca gaaggacaga |
| 1861 | cgcctgccat tgggccggat ggcgagcctc tggacgagac cagccagatg agcgacctcc |
| 1921 | cggtgaaggt gatccacgtg gagagtggga agatcctcac aggcacagat gcccccaaag |
| 1981 | ccgggcagct ggaggcctgg ctcgagatga acccggggta tgaagtagct ccgaggtctg |
| 2041 | atagtgaaga aagtggctca gaagaagagg aagaggagga ggaggaagag cagccgcagg |
| 2101 | cagcacagcc tcccaccctg cccgtggagg agaagaagaa gattccagat ccagacagcg |
| 2161 | atgacgtctc tgaggtggac gcgcggcaca tcattgagaa tgccaagcaa gatgtcgatg |
| 2221 | atgaatatgg cgtgtcccag gcccttgcac gtggcctgca gtcctactat gccgtggccc |
| 2281 | atgctgtcac tgagagagtg gacaagcagt cagcgcttat ggtcaatggt gtcctcaaac |
| 2341 | agtaccagat caaaggtttg gagtggctgg tgtccctgta caacaacaac ctgaacggca |
| 2401 | tcctggccga cgagatgggc ctggggaaga ccatccagac catcgcgctc atcacgtacc |
| 2461 | tcatggagca caaacgcatc aatgggccct tcctcatcat cgtgcctctc tcaacgctgt |
| 2521 | ccaactgggc gtacgagttt gacaagtggg ccccctccgt ggtgaaggtg tcttacaagg |
| 2581 | gatccccagc agcaagacgg gcctttgtcc cccagctccg gagtgggaag ttcaacgtct |
| 2641 | tgctgacgac gtacgagtac atcatcaaag acaagcacat cctcgccaag atccgttgga |
| 2701 | agtacatgat tgtggacgaa ggtcaccgca tgaagaacca ccactgcaag ctgacgcagg |
| 2761 | tgctcaacac gcactatgtg gcaccccgcc gcctgctgct gacgggcaca ccgctgcaga |
| 2821 | acaagcttcc cgagctctgg gcgctgctca acttcctgct gcccaccatc ttcaagagct |
| 2881 | gcagcacctt cgagcagtgg tttaacgcac cctttgccat gaccggggaa aaggtggacc |
| 2941 | tgaatgagga ggaaaccatt ctcatcatcc ggcgtctcca caaagtgctg cggcccttct |
| 3001 | tgctccgacg actcaagaag gaagtcgagg cccagttgcc cgaaaaggtg gagtacgtca |
| 3061 | tcaagtgcga catgtctgcg ctgcagcgag tgctctaccg ccacatgcag gccaagggcg |
| 3121 | tgctgctgac tgatggctcc gagaaggaca agaagggcaa aggcggcacc aagaccctga |
| 3181 | tgaacaccat catgcagctg cggaagatct gcaaccaccc ctacatgttc cagcacatcg |
| 3241 | aggagtcctt ttccgagcac ttggggttca ctggcggcat tgtccaaggg ctggacctgt |
| 3301 | accgagcctc gggtaaattt gagcttcttg atagaattct tcccaaactc cgagcaacca |
| 3361 | accacaaagt gctgctgttc tgccaaatga cctccctcat gaccatcatg gaagattact |
| 3421 | ttgcgtatcg cggctttaaa tacctcaggc ttgatggaac cacgaaggcg gaggaccggg |
| 3481 | gcatgctgct gaaaaccttc aacgagcccg gctctgagta cttcatcttc ctgctcagca |
| 3541 | cccgggctgg ggggctcggc ctgaacctcc agtcggcaga cactgtgatc atttttgaca |
| 3601 | gcgactggaa tcctcaccag gacctgcaag cgcaggaccg agcccaccgc atcgggcagc |
| 3661 | agaacgaggt gcgtgtgctc cgcctctgca ccgtcaacag cgtggaggag aagatcctag |
| 3721 | ctgcagccaa gtacaagctc aacgtggacc agaaggtgat ccaggccggc atgttcgacc |
| 3781 | agaagtcctc cagccatgag cggcgcgcct tcctgcaggc catcctggag cacgaggagc |
| 3841 | aggatgagag cagacactgc agcacgggca gcggcagtgc cagcttcgcc cacactgccc |
| 3901 | ctccgccagc gggcgtcaac cccgacttgg aggagccacc tctaaaggag gaagacgagg |
| 3961 | tgcccgacga cgagaccgtc aaccagatga tcgcccggca cgaggaggag tttgatctgt |
| 4021 | tcatgcgcat ggacctggac cgcaggcgcg aggaggcccg caaccccaag cggaagccgc |
| 4081 | gcctcatgga ggaggacgag ctcccctcgt ggatcatcaa ggacgacgcg gaggtggagc |
| 4141 | ggctgacctg tgaggaggag gaggagaaga tgttcggccg tggctcccgc caccgcaagg |
| 4201 | aggtggacta cagcgactca ctgacggaga agcagtggct caagaaaatt acaggaaaag |
| 4261 | atatccatga cacagccagc agtgtggcac gtgggctaca attccagcgt ggccttcagt |
| 4321 | tctgcacacg tgcgtcaaag gccatcgagg agggcacgct ggaggagatc gaagaggagg |
| 4381 | tccggcagaa gaaatcatca cggaagcgca agcgagacag cgacgccggc tcctccaccc |
| 4441 | cgaccaccag cacccgcagc cgcgacaagg acgacgagag caagaagcag aagaagcgcg |
| 4501 | ggcggccgcc tgccgagaaa ctctccccta acccacccaa cctcaccaag aagatgaaga |
| 4561 | agattgtgga tgccgtgatc aagtacaagg acagcagcag tggacgtcag ctcagcgagg |
| 4621 | tcttcatcca gctgccctcg cgaaaggagc tgcccgagta ctacgagctc atccgcaagc |
| 4681 | ccgtggactt caagaagata aaggagcgca ttcgcaacca caagtaccgc agcctcaacg |
| 4741 | acctagagaa ggacgtcatg ctcctgtgcc agaacgcaca gaccttcaac ctggagggct |
| 4801 | ccctgatcta tgaagactcc atcgtcttgc agtcggtctt caccagcgtg cggcagaaaa |
| 4861 | tcgagaagga ggatgacagt gaaggcgagg agagtgagga ggaggaagag ggcgaggagg |
| 4921 | aaggctccga atccgaatct cggtccgtca aagtgaagat caagcttggc cggaaggaga |
| 4981 | aggcacagga ccggctgaag ggcggccggc ggcggccgag ccgagggtcc cgagccaagc |
| 5041 | cggtcgtgag tgacgatgac agtgaggagg aacaagagga ggaccgctca ggaagtggca |
| 5101 | gcgaagaaga ctgagccccg acattccagt ctcgaccccg agcccctcgt tccagagctg |
| 5161 | agatggcata ggccttagca gtaacgggta gcagcagatg tagtttcaga cttggagtaa |
| 5221 | aactgtataa acaaaagaat cttccatatt tatacagcag agaagctgta ggactgtttg |
| 5281 | tgactggccc tgtcctggca tcagtagcat ctgtaacagc attaactgtc ttaaagagag |
| 5341 | agagagagaa ttccgaattg gggaacacac gatacctgtt tttcttttcc gttgctggca |
| 5401 | gtactgttgc gccgcagttt ggagtcactg tagttaagtg tggatgcatg tgcgtcaccg |
| 5461 | tccactcctc ctactgtatt ttattggaca ggtcagactc gccgggggcc cggcgagggt |
| 5521 | atgtcagtgt cactggatgt caaacagtaa taaattaaac caacaacaaa acgcacagcc |
| 5581 | aaaaaaaaa |

SEQ ID NO: 193 Human SMARCA4 Amino Acid Sequence Isoform B
(NP_001122316.1 and NP_003063.2)

| | |
|---|---|
| 1 | mstpdpplgg tprpgpspgp gpspgamlgp spgpspgsah smmgpspgpp saghpiptqg |
| 61 | pggypqdnmh qmhkpmesmh ekgmsddpry nqmkgmgmrs gghagmgppp spmdqhsqgy |
| 121 | psplggseha sspvpasgps sgpqmssgpg gapldgadpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa pmvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfkeyh rsvtgkiqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqaaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdesr |
| 1261 | hcstgsgsas fahtapppag vnpdleeppl keedevpdde tvnqmiarhe eefdlfmrmd |
| 1321 | ldrrreearn pkrkprlmee delpswiikd daeverltce eeeekmfgrg srhrkevdys |
| 1381 | dsltekqwlk aieegtleei eeevrqkkss rkrkrdsdag sstpttstrs rdkddeskkq |
| 1441 | kkrgrppaek lspnppnltk kmkkivdavi kykdsssgrq lsevfiqlps rkelpeyyel |
| 1501 | irkpvdfkki kerirnhkyr slndlekdvm llcqnaqtfn legsliyeds ivlqsvftsv |
| 1561 | rqkiekedds egeeseeeee geeegseses rsvkvkiklg rkekagdrlk ggrrrpsrgs |
| 1621 | rakpvvsddd seeeqeedrs gsgseed |

SEQ ID NO: 194 Human SMARCA4 cDNA Sequence Variant 2 (NM_001128844.1,
CDS: 361-5304)

| | |
|---|---|
| 1 | ggagaggccg ccgcggtgct gagggggagg ggagccggcg agcgcgcgcg cagcgggggc |
| 61 | gcgggtggcg cgcgtgtgtg tgaagggggg gcggtggccg aggcgggcgg gcgcgcgcgc |
| 121 | gaggcttccc ctcgtttggc ggcggcggcg gcttctttgt ttcgtgaaga gaagcgagac |
| 181 | gcccattctg cccccggccc cgcgcggagg ggcgggggag gcgccgggaa gtcgacggcg |
| 241 | ccggcggctc ctgcgtctcg cccttttgcc caggctagag tgcagtggtg cggtcatggt |
| 301 | tcactgcagc ctcaacctcc tggactcagc aggaggccac tgtctgcagc tcccgtgaag |
| 361 | atgtccactc cagacccacc cctgggcgga actcctcggc caggtccttc cccgggccct |
| 421 | ggcccttccc ctggagccat gctgggccct agcccgggtc cctcgccggg ctccgcccac |
| 481 | agcatgatgg ggcccagccc agggccgccc tcagcaggac accccatccc cacccagggg |
| 541 | cctggagggt accctcagga caacatgcac cagatgcaca agcccatgga gtccatgcat |
| 601 | gagaagggca tgtcggacga cccgcgctac aaccagatga aaggaatggg gatgcggtca |
| 661 | gggggccatg ctgggatggg gcccccgccc agccccatgg accagcactc ccaaggttac |
| 721 | ccctcgcccc tgggtggctc tgagcatgcc tctagtccag ttccagccag tggcccgtct |
| 781 | tcggggcccc agatgtcttc cgggccagga ggtgccccgc tggatggtgc tgacccccag |
| 841 | gccttggggc agcagaaccg gggcccaacc ccatttaacc agaaccagct gcaccagctc |
| 901 | agagctcaga tcatggccta caagatgctg gccagggggc agcccctccc cgaccacctg |
| 961 | cagatggcgg tgcagggcaa gcggccgatg cccgggatgc agcagcagat gccaacgcta |
| 1021 | cctccaccct cggtgtccgc aacaggaccc ggccctggcc ctggccctgg ccccggcccg |
| 1081 | ggtcccggcc cggcacctcc aaattacagc aggcctcatg gtatgggagg gcccaacatg |
| 1141 | cctcccccag gaccctcggg cgtgcccccc gggatgccag gccagcctcc tggagggcct |
| 1201 | cccaagccct ggcctgaagg acccatggcg aatgctgctg cccccacgag cacccctcag |
| 1261 | aagctgattc ccccgcagcc aacgggccgc ccttcccccg cgccccctgc cgtcccaccc |
| 1321 | gccgcctcgc ccgtgatgcc accgcagacc cagtcccccg ggcagccggc ccagcccgcg |
| 1381 | cccatggtgc cactgcacca gaagcagagc cgcatcaccc ccatccagaa gccgcggggc |
| 1441 | ctcgaccctg tggagatcct gcaggagcgc gagtacaggc tgcaggctcg catcgcacac |
| 1501 | cgaattcagg aacttgaaaa ccttcccggg tccctggccg gggatttgcg aaccaaagcg |
| 1561 | accattgagc tcaaggccct caggctgctg aacttccaga ggcagctgcg ccaggaggtg |
| 1621 | gtggtgtgca tgcggaggga cacagcgctg gagacagccc tcaatgctaa ggcctacaag |
| 1681 | cgcagcaagc gccagtccct gcgcgaggcc cgcatcactg agaagctgga gaagcagcag |
| 1741 | aagatcgagc aggagcgcaa gcgccggcag aagcaccagg aatacctcaa tagcattctc |
| 1801 | cagcatgcca aggatttcaa ggaatatcac agatccgtca caggcaaaat ccagaagctg |
| 1861 | accaaggcag tggccacgta ccatgccaac acggagcggg agcagaagaa agagaacgag |
| 1921 | cggatcgaga aggagcgcat gcggaggctc atggctgaag atgaggaggg gtaccgcaag |
| 1981 | ctcatcgacc agaagaagga caagcgcctg gcctacctct tgcagcagac agacgagtac |
| 2041 | gtggctaacc tcacggagct ggtgcggcag cacaaggctg cccaggtcgc caaggagaaa |
| 2101 | aagaagaaaa agaaaaagaa gaaggcagaa aatgcagaag gacagacgcc tgccattggg |
| 2161 | ccggatggcg agcctctgga cgagaccagc cagatgagcg acctcccggt gaaggtgatc |
| 2221 | cacgtggaga gtgggaagat cctcacaggc acagatgccc ccaaagccgg gcagctggag |
| 2281 | gcctggctcg agatgaaccc ggggtatgaa gtagctccga ggtctgatag tgaagaaagt |
| 2341 | ggctcagaag aagaggaaga ggaggaggag gaagagcagc cgcaggcagc acagcctccc |
| 2401 | accctgcccg tggaggagaa gaagaagatt ccagatccag acagcgatga cgtctctgag |
| 2461 | gtggacgcgc ggcacatcat tgagaatgcc aagcaagatg tcgatgatga atatggcgtg |
| 2521 | tcccaggccc ttgcacgtgg cctgcagtcc tactatgccg tggcccatgc tgtcactgag |
| 2581 | agagtggaca agcagtcagc gcttatggtc aatggtgtcc tcaaacagta ccagatcaaa |
| 2641 | ggtttggagt ggctggtgtc cctgtacaac aacaacctga acggcatcct ggccgacgag |
| 2701 | atgggcctgg ggaagaccat ccagaccatc gcgctcatca cgtacctcat ggagcacaaa |
| 2761 | cgcatcaatg ggcccttcct catcatcgtg cctctctcaa cgctgtccaa ctgggcgtac |
| 2821 | gagtttgaca agtgggcccc ctccgtggtg aaggtgtctt acaagggatc cccagcagca |
| 2881 | agacgggcct ttgtccccca gctccggagt gggaagttca acgtcttgct gacgacgtac |
| 2941 | gagtacatca tcaaagacaa gcacatcctc gccaagatcc gttggaagta catgattgtg |
| 3001 | gacgaaggtc accgcatgaa gaaccaccac tgcaagctga cgcaggtgct caacacgcac |
| 3061 | tatgtggcac cccgccgcct gctgctgacg ggcacaccgc tgcagaacaa gcttcccgag |
| 3121 | ctctgggcgc tgctcaactt cctgctgccc accatcttca agagctgcag caccttcgag |
| 3181 | cagtggttta acgcaccctt tgccatgacc ggggaaaagg tggacctgaa tgaggaggaa |
| 3241 | accattctca tcatccggcg tctccacaaa gtgctgcggc ccttcttgct ccgacgactc |
| 3301 | aagaaggaag tcgaggccca gttgcccgaa aaggtggagt acgtcatcaa gtgcgacatg |
| 3361 | tctgcgctgc agcgagtgct ctaccgccac atgcaggcca agggcgtgct gctgactgat |
| 3421 | ggctccgaga aggacaagaa gggcaaaggc ggcaccaaga ccctgatgaa caccatcatg |
| 3481 | cagctgcgga agatctgcaa ccacccctac atgttccagc acatcgagga gtccttttcc |
| 3541 | gagcacttgg ggttcactgg cggcattgtc caagggctgg acctgtaccg agcctcgggt |
| 3601 | aaatttgagc ttcttgatag aattcttccc aaactccgag caaccaacca caaagtgctg |
| 3661 | ctgttctgcc aaatgacctc cctcatgacc atcatggaag attactttgc gtatcgcggc |
| 3721 | tttaaatacc tcaggcttga tggaaccacg aaggcggagg accggggcat gctgctgaaa |
| 3781 | accttcaacg agcccggctc tgagtacttc atcttcctgc tcagcacccg ggctgggggg |
| 3841 | ctcggcctga acctccagtc ggcagacact gtgatcattt ttgacagcga ctggaatcct |
| 3901 | caccaggacc tgcaagcgca ggaccgagcc caccgcatcg ggcagcagaa cgaggtgcgt |
| 3961 | gtgctccgcc tctgcaccgt caacagcgtg gaggagaaga tcctagctgc agccaagtac |
| 4021 | aagctcaacg tggaccagaa ggtgatccag gccggcatgt tcgaccagaa gtcctccagc |
| 4081 | catgagcggc gcgccttcct gcaggccatc ctggagcacg aggagcagga tgagagcaga |
| 4141 | cactgcagca cgggcagcgg cagtgccagc ttcgcccaca ctgcccctcc gccagcgggc |
| 4201 | gtcaaccccg acttggagga gccacctcta aaggaggaag acgaggtgcc cgacgacgag |
| 4261 | accgtcaacc agatgatcgc ccggcacgag gaggagtttg atctgttcat gcgcatggac |
| 4321 | ctggaccgca ggcgcgagga ggcccgcaac cccaagcgga agccgcgcct catggaggag |
| 4381 | gacgagctcc cctcgtggat catcaaggac gacgcggagg tggagcggct gacctgtgag |
| 4441 | gaggaggagg agaagatgtt cggccgtggc tcccgccacc gcaaggaggt ggactacagc |
| 4501 | gactcactga cggagaagca gtggctcaag gccatcgagg agggcacgct ggaggagatc |
| 4561 | gaagaggagg tccggcagaa gaaatcatca cggaagcgca agcgagacag cgacgccggc |
| 4621 | tcctccaccc cgaccaccag cacccgcagc cgcgacaagg acgacgagag caagaagcag |
| 4681 | aagaagcgcg ggcggccgcc tgccgagaaa ctctccccta acccacccaa cctcaccaag |
| 4741 | aagatgaaga agattgtgga tgccgtgatc aagtacaagg acagcagcag tggacgtcag |
| 4801 | ctcagcgagg tcttcatcca gctgccctcg cgaaaggagc tgcccgagta ctacgagctc |
| 4861 | atccgcaagc ccgtggactt caagaagata aaggagcgca ttcgcaacca caagtaccgc |
| 4921 | agcctcaacg acctagagaa ggacgtcatg ctcctgtgcc agaacgcaca gaccttcaac |
| 4981 | ctggagggct ccctgatcta tgaagactcc atcgtcttgc agtcggtctt caccagcgtg |
| 5041 | cggcagaaaa tcgagaagga ggatgacagt gaaggcgagg agagtgagga ggaggaagag |
| 5101 | ggcgaggagg aaggctccga atccgaatct cggtccgtca aagtgaagat caagcttggc |
| 5161 | cggaaggaga aggcacagga ccggctgaag ggcggccggc ggcggccgag ccgagggtcc |
| 5221 | cgagccaagc cggtcgtgag tgacgatgac agtgaggagg aacaagagga ggaccgctca |
| 5281 | ggaagtggca gcgaagaaga ctgagccccg acattccagt ctcgaccccg agcccctcgt |
| 5341 | tccagagctg agatggcata ggccttagca gtaacgggta gcagcagatg tagtttcaga |
| 5401 | cttggagtaa aactgtataa acaaaagaat cttccatatt tatacagcag agaagctgta |
| 5461 | ggactgtttg tgactggccc tgtcctggca tcagtagcat ctgtaacagc attaactgtc |
| 5521 | ttaaagagag agagagagaa ttccgaattg gggaacacac gatacctgtt tttcttttcc |
| 5581 | gttgctggca gtactgttgc gccgcagttt ggagtcactg tagttaagtg tggatgcatg |
| 5641 | tgcgtcaccg tccactcctc ctactgtatt ttattggaca ggtcagactc gccgggggcc |
| 5701 | cggcgagggt atgtcagtgt cactggatgt caaacagtaa taaattaaac caacaacaaa |
| 5761 | acgcacagcc aaaaaaaaa |

SEQ ID NO: 195 Human SMARCA4 cDNA Sequence Variant 3 (NM_003072.3,
CDS: 285-5228)

| | |
|---|---|
| 1 | ggagaggccg ccgcggtgct gagggggagg ggagccggcg agcgcgcgcg cagcgggggc |
| 61 | gcgggtggcg cgcgtgtgtg tgaagggggg gcggtggccg aggcgggcgg gcgcgcgcgc |
| 121 | gaggcttccc ctcgtttggc ggcggcggcg gcttctttgt ttcgtgaaga gaagcgagac |
| 181 | gcccattctg cccccggccc cgcgcggagg ggcgggggag gcgccgggaa gtcgacggcg |
| 241 | ccggcggctc ctgcaggagg ccactgtctg cagctcccgt gaagatgtcc actccagacc |
| 301 | cacccctggg cggaactcct cggccaggtc cttccccggg ccctggccct tcccctggag |
| 361 | ccatgctggg ccctagcccg ggtccctcgc cgggctccgc ccacagcatg atggggccca |
| 421 | gcccagggcc gccctcagca ggacacccca tccccaccca ggggcctgga gggtaccctc |
| 481 | aggacaacat gcaccagatg cacaagccca tggagtccat gcatgagaag ggcatgtcgg |
| 541 | acgacccgcg ctacaaccag atgaaaggaa tggggatgcg gtcagggggc catgctggga |
| 601 | tggggccccc gcccagcccc atggaccagc actcccaagg ttacccctcg cccctgggtg |
| 661 | gctctgagca tgcctctagt ccagttccag ccagtggccc gtcttcgggg ccccagatgt |
| 721 | cttccgggcc aggaggtgcc ccgctggatg gtgctgaccc ccaggccttg gggcagcaga |
| 781 | accggggccc aaccccattt aaccagaacc agctgcacca gctcagagct cagatcatgg |
| 841 | cctacaagat gctggccagg gggcagcccc tccccgacca cctgcagatg gcggtgcagg |
| 901 | gcaagcggcc gatgcccggg atgcagcagc agatgccaac gctacctcca ccctcggtgt |
| 961 | ccgcaacagg acccggccct ggccctggcc ctggccccgg cccgggtccc ggcccggcac |
| 1021 | ctccaaatta cagcaggcct catggtatgg gagggcccaa catgcctccc ccaggaccct |
| 1081 | cgggcgtgcc ccccgggatg ccaggccagc ctcctggagg gcctcccaag ccctggcctg |
| 1141 | aaggacccat ggcgaatgct gctgccccca cgagcacccc tcagaagctg attcccccgc |
| 1201 | agccaacggg ccgcccttcc cccgcgcccc ctgccgtccc acccgccgcc tcgcccgtga |
| 1261 | tgccaccgca gacccagtcc cccgggcagc cggcccagcc cgcgcccatg gtgccactgc |
| 1321 | accagaagca gagccgcatc acccccatcc agaagccgcg gggcctcgac cctgtggaga |
| 1381 | tcctgcagga gcgcgagtac aggctgcagg ctcgcatcgc acaccgaatt caggaacttg |
| 1441 | aaaaccttcc cgggtccctg gccggggatt tgcgaaccaa agcgaccatt gagctcaagg |
| 1501 | ccctcaggct gctgaacttc cagaggcagc tgcgccagga ggtggtggtg tgcatgcgga |
| 1561 | gggacacagc gctggagaca gccctcaatg ctaaggccta caagcgcagc aagcgccagt |
| 1621 | ccctgcgcga ggcccgcatc actgagaagc tggagaagca gcagaagatc gagcaggagc |
| 1681 | gcaagcgccg gcagaagcac caggaatacc tcaatagcat tctccagcat gccaaggatt |
| 1741 | tcaaggaata tcacagatcc gtcacaggca aaatccagaa gctgaccaag gcagtggcca |
| 1801 | cgtaccatgc caacacggag cgggagcaga agaaagagaa cgagcggatc gagaaggagc |
| 1861 | gcatgcggag gctcatggct gaagatgagg aggggtaccg caagctcatc gaccagaaga |
| 1921 | aggacaagcg cctggcctac ctcttgcagc agacagacga gtacgtggct aacctcacgg |
| 1981 | agctggtgcg gcagcacaag gctgcccagg tcgccaagga gaaaaagaag aaaaagaaaa |
| 2041 | agaagaaggc agaaaatgca gaaggacaga cgcctgccat tgggccggat ggcgagcctc |
| 2101 | tggacgagac cagccagatg agcgacctcc cggtgaaggt gatccacgtg gagagtggga |
| 2161 | agatcctcac aggcacagat gcccccaaag ccgggcagct ggaggcctgg ctcgagatga |
| 2221 | acccggggta tgaagtagct ccgaggtctg atagtgaaga aagtggctca gaagaagagg |
| 2281 | aagaggagga ggaggaagag cagccgcagg cagcacagcc tcccaccctg cccgtggagg |
| 2341 | agaagaagaa gattccagat ccagacagcg atgacgtctc tgaggtggac gcgcggcaca |
| 2401 | tcattgagaa tgccaagcaa gatgtcgatg atgaatatgg cgtgtcccag gcccttgcac |
| 2461 | gtggcctgca gtcctactat gccgtggccc atgctgtcac tgagagagtg gacaagcagt |
| 2521 | cagcgcttat ggtcaatggt gtcctcaaac agtaccagat caaaggtttg gagtggctgg |
| 2581 | tgtccctgta caacaacaac ctgaacggca tcctggccga cgagatgggc ctggggaaga |
| 2641 | ccatccagac catcgcgctc atcacgtacc tcatggagca caaacgcatc aatgggccct |
| 2701 | tcctcatcat cgtgcctctc tcaacgctgt ccaactgggc gtacgagttt gacaagtggg |
| 2761 | ccccctccgt ggtgaaggtg tcttacaagg gatccccagc agcaagacgg gcctttgtcc |
| 2821 | cccagctccg gagtgggaag ttcaacgtct tgctgacgac gtacgagtac atcatcaaag |
| 2881 | acaagcacat cctcgccaag atccgttgga agtacatgat tgtggacgaa ggtcaccgca |
| 2941 | tgaagaacca ccactgcaag ctgacgcagg tgctcaacac gcactatgtg gcaccccgcc |
| 3001 | gcctgctgct gacgggcaca ccgctgcaga acaagcttcc cgagctctgg gcgctgctca |
| 3061 | acttcctgct gcccaccatc ttcaagagct gcagcacctt cgagcagtgg tttaacgcac |
| 3121 | cctttgccat gaccggggaa aaggtggacc tgaatgagga ggaaaccatt ctcatcatcc |
| 3181 | ggcgtctcca caaagtgctg cggcccttct tgctccgacg actcaagaag gaagtcgagg |
| 3241 | cccagttgcc cgaaaaggtg gagtacgtca tcaagtgcga catgtctgcg ctgcagcgag |
| 3301 | tgctctaccg ccacatgcag gccaagggcg tgctgctgac tgatggctcc gagaaggaca |
| 3361 | agaagggcaa aggcggcacc aagaccctga tgaacaccat catgcagctg cggaagatct |
| 3421 | gcaaccaccc ctacatgttc cagcacatcg aggagtcctt ttccgagcac ttggggttca |
| 3481 | ctggcggcat tgtccaaggg ctggacctgt accgagcctc gggtaaattt gagcttcttg |
| 3541 | atagaattct tcccaaactc cgagcaacca accacaaagt gctgctgttc tgccaaatga |
| 3601 | cctccctcat gaccatcatg gaagattact ttgcgtatcg cggctttaaa tacctcaggc |
| 3661 | ttgatggaac cacgaaggcg gaggaccggg gcatgctgct gaaaaccttc aacgagcccg |
| 3721 | gctctgagta cttcatcttc ctgctcagca cccgggctgg ggggctcggc ctgaacctcc |
| 3781 | agtcggcaga cactgtgatc atttttgaca gcgactggaa tcctcaccag gacctgcaag |
| 3841 | cgcaggaccg agcccaccgc atcgggcagc agaacgaggt gcgtgtgctc cgcctctgca |
| 3901 | ccgtcaacag cgtggaggag aagatcctag ctgcagccaa gtacaagctc aacgtggacc |
| 3961 | agaaggtgat ccaggccggc atgttcgacc agaagtcctc cagccatgag cggcgcgcct |
| 4021 | tcctgcaggc catcctggag cacgaggagc aggatgagag cagacactgc agcacgggca |
| 4081 | gcggcagtgc cagcttcgcc cacactgccc ctccgccagc gggcgtcaac cccgacttgg |
| 4141 | aggagccacc tctaaaggag gaagacgagg tgcccgacga cgagaccgtc aaccagatga |
| 4201 | tcgcccggca cgaggaggag tttgatctgt tcatgcgcat ggacctggac cgcaggcgcg |
| 4261 | aggaggcccg caaccccaag cggaagccgc gcctcatgga ggaggacgag ctcccctcgt |
| 4321 | ggatcatcaa ggacgacgcg gaggtggagc ggctgacctg tgaggaggag gaggagaaga |
| 4381 | tgttcggccg tggctcccgc caccgcaagg aggtggacta cagcgactca ctgacggaga |
| 4441 | agcagtggct caaggccatc gaggagggca cgctggagga gatcgaagag gaggtccggc |
| 4501 | agaagaaatc atcacggaag cgcaagcgag acagcgacgc cggctcctcc accccgacca |
| 4561 | ccagcacccg cagccgcgac aaggacgacg agagcaagaa gcagaagaag cgcgggcggc |
| 4621 | cgcctgccga gaaactctcc cctaacccac ccaacctcac caagaagatg aagaagattg |
| 4681 | tggatgccgt gatcaagtac aaggacagca gcagtggacg tcagctcagc gaggtcttca |
| 4741 | tccagctgcc ctcgcgaaag gagctgcccg agtactacga gctcatccgc aagcccgtgg |
| 4801 | acttcaagaa gataaaggag cgcattcgca accacaagta ccgcagcctc aacgacctag |
| 4861 | agaaggacgt catgctcctg tgccagaacg cacagacctt caacctggag ggctccctga |
| 4921 | tctatgaaga ctccatcgtc ttgcagtcgg tcttcaccag cgtgcggcag aaaatcgaga |
| 4981 | aggaggatga cagtgaaggc gaggagagtg aggaggagga agagggcgag gaggaaggct |
| 5041 | ccgaatccga atctcggtcc gtcaaagtga agatcaagct tggccggaag gagaaggcac |
| 5101 | aggaccggct gaagggcggc cggcggcggc cgagccgagg gtcccgagcc aagccggtcg |
| 5161 | tgagtgacga tgacagtgag gaggaacaag aggaggaccg ctcaggaagt ggcagcgaag |
| 5221 | aagactgagc cccgacattc cagtctcgac cccgagcccc tcgttccaga gctgagatgg |
| 5281 | cataggcctt agcagtaacg ggtagcagca gatgtagttt cagacttgga gtaaaactgt |
| 5341 | ataaacaaaa gaatcttcca tatttataca gcagagaagc tgtaggactg tttgtgactg |
| 5401 | gccctgtcct ggcatcagta gcatctgtaa cagcattaac tgtcttaaag agagagagag |
| 5461 | agaattccga attggggaac acacgatacc tgtttttctt ttccgttgct ggcagtactg |
| 5521 | ttgcgccgca gtttggagtc actgtagtta agtgtggatg catgtgcgtc accgtccact |
| 5581 | cctcctactg tattttattg gacaggtcag actcgccggg ggcccggcga gggtatgtca |
| 5641 | gtgtcactgg atgtcaaaca gtaataaatt aaaccaacaa caaaacgcac agccaaaaaa |
| 5701 | aaa |

SEQ ID NO: 196 Human SMARCA4 Amino Acid Sequence Isoform C
(NP_001122317.1)

| | |
|---|---|
| 1 | mstpdpplgg tprpgpspgp gpspgamlgp spgpspgsah smmgpspgpp saghpiptqg |
| 61 | pggypqdnmh qmhkpmesmh ekgmsddpry nqmkgmgmrs gghagmgppp spmdqhsqgy |
| 121 | psplggseha sspvpasgps sgpqmssgpg gapldgadpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa pmvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfkeyh rsvtgkiqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqaaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlktlk aieegtleei eeevrqkkss |
| 1381 | rkrkrdsdag sstpttstrs rdkddeskkq kkrgrppaek lspnppnltk kmkkivdavi |
| 1441 | kykdsssgrq lsevfiqlps rkelpeyyel irkpvdfkki kerirnhkyr slndlekdvm |
| 1501 | llcqnaqtfn legsliyeds ivlqsvftsv rqkiekedds egeeseeeee geeegseses |
| 1561 | rsvkvkiklg rkekaqdrlk ggrrrpsrgs rakpvvsddd seeeqeedrs gsgseed |

SEQ ID NO: 197 Human SMARCA4 cDNA Sequence Variant 4 (NM_001128845.1,
CDS: 1-4854)

| | |
|---|---|
| 1 | atgtccactc cagacccacc cctgggcgga actcctcggc caggtccttc cccgggccct |
| 61 | ggcccttccc ctggagccat gctgggccct agcccgggtc cctcgccggg ctccgcccac |
| 121 | agcatgatgg ggcccagccc agggccgccc tcagcaggac accccatccc cacccagggg |
| 181 | cctggagggt accctcagga caacatgcac cagatgcaca agcccatgga gtccatgcat |
| 241 | gagaagggca tgtcggacga cccgcgctac aaccagatga aaggaatggg gatgcggtca |
| 301 | gggggccatg ctgggatggg gcccccgccc agccccatgg accagcactc ccaaggttac |
| 361 | ccctcgcccc tgggtggctc tgagcatgcc tctagtccag ttccagccag tggcccgtct |
| 421 | tcggggcccc agatgtcttc cgggccagga ggtgccccgc tggatggtgc tgacccccag |
| 481 | gccttggggc agcagaaccg gggcccaacc ccatttaacc agaaccagct gcaccagctc |
| 541 | agagctcaga tcatggccta caagatgctg gccagggggc agcccctccc cgaccacctg |
| 601 | cagatggcgg tgcagggcaa gcggccgatg cccgggatgc agcagcagat gccaacgcta |
| 661 | cctccaccct cggtgtccgc aacaggaccc ggccctggcc ctggccctgg ccccggcccg |
| 721 | ggtcccggcc cggcacctcc aaattacagc aggcctcatg gtatgggagg gcccaacatg |
| 781 | cctcccccag gaccctcggg cgtgcccccc gggatgccag gccagcctcc tggagggcct |
| 841 | cccaagccct ggcctgaagg acccatggcg aatgctgctg cccccacgag cacccctcag |
| 901 | aagctgattc ccccgcagcc aacgggccgc ccttcccccg cgccccctgc cgtcccaccc |
| 961 | gccgcctcgc ccgtgatgcc accgcagacc cagtcccccg ggcagccggc ccagcccgcg |
| 1021 | cccatggtgc cactgcacca gaagcagagc cgcatcaccc ccatccagaa gccgcggggc |
| 1081 | ctcgaccctg tggagatcct gcaggagcgc gagtacaggc tgcaggctcg catcgcacac |
| 1141 | cgaattcagg aacttgaaaa ccttcccggg tccctggccg gggatttgcg aaccaaagcg |
| 1201 | accattgagc tcaaggccct caggctgctg aacttccaga ggcagctgcg ccaggaggtg |
| 1261 | gtggtgtgca tgcggaggga cacagcgctg gagacagccc tcaatgctaa ggcctacaag |
| 1321 | cgcagcaagc gccagtccct gcgcgaggcc cgcatcactg agaagctgga gaagcagcag |
| 1381 | aagatcgagc aggagcgcaa gcgccggcag aagcaccagg aatacctcaa tagcattctc |
| 1441 | cagcatgcca aggatttcaa ggaatatcac agatccgtca caggcaaaat ccagaagctg |
| 1501 | accaaggcag tggccacgta ccatgccaac acggagcggg agcagaagaa agagaacgag |
| 1561 | cggatcgaga aggagcgcat gcggaggctc atggctgaag atgaggaggg gtaccgcaag |
| 1621 | ctcatcgacc agaagaagga caagcgcctg gcctacctct tgcagcagac agacgagtac |
| 1681 | gtggctaacc tcacggagct ggtgcggcag cacaaggctg cccaggtcgc caaggagaaa |
| 1741 | aagaagaaaa agaaaaagaa gaaggcagaa aatgcagaag gacagacgcc tgccattggg |
| 1801 | ccggatggcg agcctctgga cgagaccagc cagatgagcg acctcccggt gaaggtgatc |
| 1861 | cacgtggaga gtgggaagat cctcacaggc acagatgccc ccaaagccgg gcagctggag |
| 1921 | gcctggctcg agatgaaccc ggggtatgaa gtagctccga ggtctgatag tgaagaaagt |
| 1981 | ggctcagaag aagaggaaga ggaggaggag gaagagcagc cgcaggcagc acagcctccc |
| 2041 | accctgcccg tggaggagaa gaagaagatt ccagatccag acagcgatga cgtctctgag |
| 2101 | gtggacgcgc ggcacatcat tgagaatgcc aagcaagatg tcgatgatga atatggcgtg |
| 2161 | tcccaggccc ttgcacgtgg cctgcagtcc tactatgccg tggcccatgc tgtcactgag |
| 2221 | agagtggaca agcagtcagc gcttatggtc aatggtgtcc tcaaacagta ccagatcaaa |
| 2281 | ggtttggagt ggctggtgtc cctgtacaac aacaacctga acggcatcct ggccgacgag |
| 2341 | atgggcctgg ggaagaccat ccagaccatc gcgctcatca cgtacctcat ggagcacaaa |
| 2401 | cgcatcaatg ggcccttcct catcatcgtg cctctctcaa cgctgtccaa ctgggcgtac |
| 2461 | gagtttgaca agtgggcccc ctccgtggtg aaggtgtctt acaagggatc cccagcagca |
| 2521 | agacgggcct ttgtccccca gctccggagt gggaagttca acgtcttgct gacgacgtac |
| 2581 | gagtacatca tcaaagacaa gcacatcctc gccaagatcc gttggaagta catgattgtg |
| 2641 | gacgaaggtc accgcatgaa gaaccaccac tgcaagctga cgcaggtgct caacacgcac |
| 2701 | tatgtggcac cccgccgcct gctgctgacg ggcacaccgc tgcagaacaa gcttcccgag |
| 2761 | ctctgggcgc tgctcaactt cctgctgccc accatcttca agagctgcag caccttcgag |
| 2821 | cagtggttta acgcaccctt tgccatgacc ggggaaaagg tggacctgaa tgaggaggaa |
| 2881 | accattctca tcatccggcg tctccacaaa gtgctgcggc ccttcttgct ccgacgactc |
| 2941 | aagaaggaag tcgaggccca gttgcccgaa aaggtggagt acgtcatcaa gtgcgacatg |
| 3001 | tctgcgctgc agcgagtgct ctaccgccac atgcaggcca agggcgtgct gctgactgat |
| 3061 | ggctccgaga aggacaagaa gggcaaaggc ggcaccaaga ccctgatgaa caccatcatg |
| 3121 | cagctgcgga agatctgcaa ccacccctac atgttccagc acatcgagga gtccttttcc |
| 3181 | gagcacttgg ggttcactgg cggcattgtc caagggctgg acctgtaccg agcctcgggt |
| 3241 | aaatttgagc ttcttgatag aattcttccc aaactccgag caaccaacca caaagtgctg |
| 3301 | ctgttctgcc aaatgacctc cctcatgacc atcatggaag attactttgc gtatcgcggc |
| 3361 | tttaaatacc tcaggcttga tggaaccacg aaggcggagg accggggcat gctgctgaaa |
| 3421 | accttcaacg agcccggctc tgagtacttc atcttcctgc tcagcacccg ggctgggggg |
| 3481 | ctcggcctga acctccagtc ggcagacact gtgatcattt ttgacagcga ctggaatcct |
| 3541 | caccaggacc tgcaagcgca ggaccgagcc caccgcatcg ggcagcagaa cgaggtgcgt |
| 3601 | gtgctccgcc tctgcaccgt caacagcgtg gaggagaaga tcctagctgc agccaagtac |
| 3661 | aagctcaacg tggaccagaa ggtgatccag gccggcatgt tcgaccagaa gtcctccagc |
| 3721 | catgagcggc gcgccttcct gcaggccatc ctggagcacg aggagcagga tgaggaggaa |
| 3781 | gacgaggtgc ccgacgacga gaccgtcaac cagatgatcg cccggcacga ggaggagttt |
| 3841 | gatctgttca tgcgcatgga cctggaccgc aggcgcgagg aggcccgcaa ccccaagcgg |
| 3901 | aagccgcgcc tcatggagga ggacgagctc ccctcgtgga tcatcaagga cgacgcggag |
| 3961 | gtggagcggc tgacctgtga ggaggaggag gagaagatgt tcggccgtgg ctcccgccac |
| 4021 | cgcaaggagg tggactacag cgactcactg acggagaagc agtggctcaa gaccctgaag |
| 4081 | gccatcgagg agggcacgct ggaggagatc gaagaggagg tccggcagaa gaaatcatca |
| 4141 | cggaagcgca agcgagacag cgacgccggc tcctccaccc cgaccaccag cacccgcagc |
| 4201 | cgcgacaagg acgacgagag caagaagcag aagaagcgcg ggcggccgcc tgccgagaaa |
| 4261 | ctctccccta acccacccaa cctcaccaag aagatgaaga agattgtgga tgccgtgatc |
| 4321 | aagtacaagg acagcagcag tggacgtcag ctcagcgagg tcttcatcca gctgccctcg |
| 4381 | cgaaaggagc tgcccgagta ctacgagctc atccgcaagc ccgtggactt caagaagata |
| 4441 | aaggagcgca ttcgcaacca caagtaccgc agcctcaacg acctagagaa ggacgtcatg |
| 4501 | ctcctgtgcc agaacgcaca gaccttcaac ctggagggct ccctgatcta tgaagactcc |
| 4561 | atcgtcttgc agtcggtctt caccagcgtg cggcagaaaa tcgagaagga ggatgacagt |
| 4621 | gaaggcgagg agagtgagga ggaggaagag ggcgaggagg aaggctccga atccgaatct |
| 4681 | cggtccgtca aagtgaagat caagcttggc cggaaggaga aggcacagga ccggctgaag |
| 4741 | ggcggccggx ggcggccgag ccgagggtcc cgagccaagc cggtcgtgag tgacgatgac |
| 4801 | agtgaggagg aacaagagga ggaccgctca ggaagtggca gcgaagaaga ctgagccccg |
| 4861 | acattccagt ctcgaccccg agcccctcgt tccagagctg agatggcata ggccttagca |
| 4921 | gtaacgggta gcagcagatg tagtttcaga cttggagtaa aactgtataa acaaaagaat |
| 4981 | cttccatatt tatacagcag agaagctgta ggactgtttg tgactggccc tgtcctggca |
| 5041 | tcagtagcat ctgtaacagc attaactgtc ttaaagagag agagagagaa ttccgaattg |
| 5101 | gggaacacac gatacctgtt tttcttttcc gttgctggca gtactgttgc gccgcagttt |
| 5161 | ggagtcactg tagttaagtg tggatgcatg tgcgtcaccg tccactcctc ctactgtatt |
| 5221 | ttattggaca ggtcagactc gccgggggcc cggcgagggt atgtcagtgt cactggatgt |
| 5281 | caaacagtaa taaattaaac caacaacaaa acgcacagcc aaaaaaaaa |

SEQ ID NO: 198 Human SMARCA4 Amino Acid Sequence Isoform D
(NP_001122318.1)

| | |
|---|---|
| 1 | mstpdpplgg tprpgpspgp gpspgamlgp spgpspgsah smmgpspgpp saghpiptqg |
| 61 | pggypqdnmh qmhkpmesmh ekgmsddpry nqmkgmgmrs gghagmgppp spmdqhsqgy |
| 121 | psplggseha sspvpasgps sgpqmssgpg gapldgadpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa pmvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfkeyh rsvtgkiqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkaggle awlemnpgye vaprsdsees |
| 661 | gseeeeeeee eeqpqaaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlktlk aieegtleei eeevrqkkss |
| 1381 | rkrkrdsdag sstpttstrs rdkddeskkq kkrgrppaek lspnppnltk kmkkivdavi |
| 1441 | kykdssgrql sevfiqlpsr kelpeyyeli rkpvdfkkik erirnhkyrs Indlekdvml |
| 1501 | lcqnaqtfnl egsliyedsi vlqsvftsvr qkiekeddse geeseeeeeg eeegsesesr |
| 1561 | svkvkiklgr kekaqdrlkg grrrpsrgsr akpvvsddds eeeqeedrsg sgseed |

SEQ ID NO: 199 Human SMARCA4 cDNA Sequence Variant 5 (NM_001128846.1,
CDS: 1-4851)

| | |
|---|---|
| 1 | atgtccactc cagacccacc cctgggcgga actcctcggc caggtccttc cccgggccct |
| 61 | ggcccttccc ctggagccat gctgggccct agcccgggtc cctcgccggg ctccgcccac |
| 121 | agcatgatgg ggcccagccc agggccgccc tcagcaggac accccatccc cacccagggg |
| 181 | cctggagggt accctcagga caacatgcac cagatgcaca agcccatgga gtccatgcat |
| 241 | gagaagggca tgtcggacga cccgcgctac aaccagatga aaggaatggg gatgcggtca |
| 301 | gggggccatg ctgggatggg gcccccgccc agccccatgg accagcactc ccaaggttac |
| 361 | ccctcgcccc tgggtggctc tgagcatgcc tctagtccag ttccagccag tggcccgtct |
| 421 | tcggggcccc agatgtcttc cgggccagga ggtgccccgc tggatggtgc tgacccccag |
| 481 | gccttggggc agcagaaccg gggcccaacc ccatttaacc agaaccagct gcaccagctc |
| 541 | agagctcaga tcatggccta caagatgctg gccagggggc agcccctccc cgaccacctg |
| 601 | cagatggcgg tgcagggcaa gcggccgatg cccgggatgc agcagcagat gccaacgcta |
| 661 | cctccaccct cggtgtccgc aacaggaccc ggccctggcc ctggccctgg ccccggcccg |
| 721 | ggtcccggcc cggcacctcc aaattacagc aggcctcatg gtatgggagg gcccaacatg |
| 781 | cctcccccag gaccctcggg cgtgcccccc gggatgccag gccagcctcc tggagggcct |
| 841 | cccaagccct ggcctgaagg acccatggcg aatgctgctg cccccacgag cacccctcag |
| 901 | aagctgattc ccccgcagcc aacgggccgc ccttcccccg cgccccctgc cgtcccaccc |
| 961 | gccgcctcgc ccgtgatgcc accgcagacc cagtcccccg ggcagccggc ccagcccgcg |
| 1021 | cccatggtgc cactgcacca gaagcagagc cgcatcaccc ccatccagaa gccgcggggc |
| 1081 | ctcgaccctg tggagatcct gcaggagcgc gagtacaggc tgcaggctcg catcgcacac |
| 1141 | cgaattcagg aacttgaaaa ccttcccggg tccctggccg gggatttgcg aaccaaagcg |
| 1201 | accattgagc tcaaggccct caggctgctg aacttccaga ggcagctgcg ccaggaggtg |
| 1261 | gtggtgtgca tgcggaggga cacagcgctg gagacagccc tcaatgctaa ggcctacaag |
| 1321 | cgcagcaagc gccagtccct gcgcgaggcc cgcatcactg agaagctgga gaagcagcag |
| 1381 | aagatcgagc aggagcgcaa gcgccggcag aagcaccagg aatacctcaa tagcattctc |
| 1441 | cagcatgcca aggatttcaa ggaatatcac agatccgtca caggcaaaat ccagaagctg |
| 1501 | accaaggcag tggccacgta ccatgccaac acggagcggg agcagaagaa agagaacgag |
| 1561 | cggatcgaga aggagcgcat gcggaggctc atggctgaag atgaggaggg gtaccgcaag |
| 1621 | ctcatcgacc agaagaagga caagcgcctg gcctacctct tgcagcagac agacgagtac |
| 1681 | gtggctaacc tcacggagct ggtgcggcag cacaaggctg cccaggtcgc caaggagaaa |
| 1741 | aagaagaaaa agaaaaagaa gaaggcagaa aatgcagaag gacagacgcc tgccattggg |
| 1801 | ccggatggcg agcctctgga cgagaccagc cagatgagcg acctcccggt gaaggtgatc |
| 1861 | cacgtggaga gtgggaagat cctcacaggc acagatgccc ccaaagccgg gcagctggag |
| 1921 | gcctggctcg agatgaaccc ggggtatgaa gtagctccga ggtctgatag tgaagaaagt |
| 1981 | ggctcagaag aagaggaaga ggaggaggag gaagagcagc cgcaggcagc acagcctccc |
| 2041 | accctgcccg tggaggagaa gaagaagatt ccagatccag acagcgatga cgtctctgag |
| 2101 | gtggacgcgc ggcacatcat tgagaatgcc aagcaagatg tcgatgatga atatggcgtg |
| 2161 | tcccaggccc ttgcacgtgg cctgcagtcc tactatgccg tggcccatgc tgtcactgag |
| 2221 | agagtggaca agcagtcagc gcttatggtc aatggtgtcc tcaaacagta ccagatcaaa |
| 2281 | ggtttggagt ggctggtgtc cctgtacaac aacaacctga acggcatcct ggccgacgag |
| 2341 | atgggcctgg ggaagaccat ccagaccatc gcgctcatca cgtacctcat ggagcacaaa |
| 2401 | cgcatcaatg ggcccttcct catcatcgtg cctctctcaa cgctgtccaa ctgggcgtac |
| 2461 | gagtttgaca agtgggcccc ctccgtggtg aaggtgtctt acaagggatc cccagcagca |
| 2521 | agacgggcct ttgtccccca gctccggagt gggaagttca acgtcttgct gacgacgtac |
| 2581 | gagtacatca tcaaagacaa gcacatcctc gccaagatcc gttggaagta catgattgtg |
| 2641 | gacgaaggtc accgcatgaa gaaccaccac tgcaagctga cgcaggtgct caacacgcac |
| 2701 | tatgtggcac cccgccgcct gctgctgacg ggcacaccgc tgcagaacaa gcttcccgag |
| 2761 | ctctgggcgc tgctcaactt cctgctgccc accatcttca agagctgcag caccttcgag |
| 2821 | cagtggttta acgcaccctt tgccatgacc ggggaaaagg tggacctgaa tgaggaggaa |
| 2881 | accattctca tcatccggcg tctccacaaa gtgctgcggc ccttcttgct ccgacgactc |
| 2941 | aagaaggaag tcgaggccca gttgcccgaa aaggtggagt acgtcatcaa gtgcgacatg |
| 3001 | tctgcgctgc agcgagtgct ctaccgccac atgcaggcca agggcgtgct gctgactgat |
| 3061 | ggctccgaga aggacaagaa gggcaaaggc ggcaccaaga ccctgatgaa caccatcatg |
| 3121 | cagctgcgga agatctgcaa ccacccctac atgttccagc acatcgagga gtccttttcc |
| 3181 | gagcacttgg ggttcactgg cggcattgtc caagggctgg acctgtaccg agcctcgggt |
| 3241 | aaatttgagc ttcttgatag aattcttccc aaactccgag caaccaacca caaagtgctg |
| 3301 | ctgttctgcc aaatgacctc cctcatgacc atcatggaag attactttgc gtatcgcggc |
| 3361 | tttaaatacc tcaggcttga tggaaccacg aaggcggagg accggggcat gctgctgaaa |
| 3421 | accttcaacg agcccggctc tgagtacttc atcttcctgc tcagcacccg ggctgggggg |
| 3481 | ctcggcctga acctccagtc ggcagacact gtgatcattt ttgacagcga ctggaatcct |
| 3541 | caccaggacc tgcaagcgca ggaccgagcc caccgcatcg ggcagcagaa cgaggtgcgt |
| 3601 | gtgctccgcc tctgcaccgt caacagcgtg gaggagaaga tcctagctgc agccaagtac |
| 3661 | aagctcaacg tggaccagaa ggtgatccag gccggcatgt tcgaccagaa gtcctccagc |
| 3721 | catgagcggc gcgccttcct gcaggccatc ctggagcacg aggagcagga tgaggaggaa |
| 3781 | gacgaggtgc ccgacgacga gaccgtcaac cagatgatcg cccggcacga ggaggagttt |
| 3841 | gatctgttca tgcgcatgga cctggaccgc aggcgcgagg aggcccgcaa ccccaagcgg |
| 3901 | aagccgcgcc tcatggagga ggacgagctc ccctcgtgga tcatcaagga cgacgcggag |
| 3961 | gtggagcggc tgacctgtga ggaggaggag gagaagatgt tcggccgtgg ctcccgccac |
| 4021 | cgcaaggagg tggactacag cgactcactg acggagaagc agtggctcaa gaccctgaag |
| 4081 | gccatcgagg agggcacgct ggaggagatc gaagaggagg tccggcagaa gaaatcatca |
| 4141 | cggaagcgca agcgagacag cgacgccggc tcctccaccc cgaccaccag cacccgcagc |
| 4201 | cgcgacaagg acgacgagag caagaagcag aagaagcgcg ggcggccgcc tgccgagaaa |
| 4261 | ctctccccta acccacccaa cctcaccaag aagatgaaga agattgtgga tgccgtgatc |
| 4321 | aagtacaagg acagcagtgg acgtcagctc agcgaggtct tcatccagct gccctcgcga |
| 4381 | aaggagctgc ccgagtacta cgagctcatc cgcaagcccg tggacttcaa gaagataaag |
| 4441 | gagcgcattc gcaaccacaa gtaccgcagc ctcaacgacc tagagaagga cgtcatgctc |
| 4501 | ctgtgccaga acgcacagac cttcaacctg gagggctccc tgatctatga agactccatc |
| 4561 | gtcttgcagt cggtcttcac cagcgtgcgg cagaaaatcg agaaggagga tgacagtgaa |
| 4621 | ggcgaggaga gtgaggagga ggaagagggc gaggaggaag gctccgaatc cgaatctcgg |
| 4681 | tccgtcaaag tgaagatcaa gcttggccgg aaggagaagg cacaggaccg gctgaagggc |
| 4741 | ggccggcggc ggccgagccg agggtcccga gccaagccgg tcgtgagtga cgatgacagt |
| 4801 | gaggaggaac aagaggagga ccgctcagga agtggcagcg aagaagactg agccccgaca |
| 4861 | ttccagtctc gaccccgagc ccctcgttcc agagctgaga tggcataggc cttagcagta |
| 4921 | acgggtagca gcagatgtag tttcagactt ggagtaaaac tgtataaaca aaagaatctt |
| 4981 | ccatatttat acagcagaga agctgtagga ctgtttgtga ctggccctgt cctggcatca |
| 5041 | gtagcatctg taacagcatt aactgtctta aagagagaga gagagaattc cgaattgggg |
| 5101 | aacacacgat acctgttttt cttttccgtt gctggcagta ctgttgcgcc gcagtttgga |
| 5161 | gtcactgtag ttaagtgtgg atgcatgtgc gtcaccgtcc actcctccta ctgtatttta |
| 5221 | ttggacaggt cagactcgcc gggggcccgg cgagggtatg tcagtgtcac tggatgtcaa |
| 5281 | acagtaataa attaaaccaa caacaaaacg cacagccaaa aaaaaa |

SEQ ID NO: 200 Human SMARCA4 Amino Acid Sequence Isoform E
(NP_001122319.1)

| | |
|---|---|
| 1 | mstpdpplgg tprpgpspgp gpspgamlgp spgpspgsah smmgpspgpp saghpiptqg |
| 61 | pggypqdnmh qmhkpmesmh ekgmsddpry nqmkgmgmrs gghagmgppp spmdqhsqgy |
| 121 | psplggseha sspvpasgps sgpqmssgpg gapldgadpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa pmvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfkeyh rsvtgkiqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkaggle awlemnpgye vaprsdsees |
| 661 | gseeeeeeee eeqpqaaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlkaie egtleeieee vrqkkssrkr |
| 1381 | krdsdagsst pttstrsrdk ddeskkqkkr grppaeklsp nppnltkkmk kivdavikyk |
| 1441 | dsssgrqlse vfiqlpsrke lpeyyelirk pvdfkkiker irnhkyrsln dlekdvmllc |
| 1501 | qnaqtfnleg sliyedsivl qsvftsvrqk iekeddsege eseeeeegee egsesesrsv |
| 1561 | kvkiklgrke kaqdrlkggr rrpsrgsrak pvvsdddsee eqeedrsgsg seed |

SEQ ID NO: 201 Human SMARCA4 cDNA Sequence Variant 6 (NM_001128847.1,
CDS: 1-4845)

| | |
|---|---|
| 1 | atgtccactc cagacccacc cctgggcgga actcctcggc caggtccttc cccgggccct |
| 61 | ggcccttccc ctggagccat gctgggccct agcccgggtc cctcgccggg ctccgcccac |
| 121 | agcatgatgg ggcccagccc agggccgccc tcagcaggac accccatccc cacccagggg |
| 181 | cctggagggt accctcagga caacatgcac cagatgcaca agcccatgga gtccatgcat |
| 241 | gagaagggca tgtcggacga cccgcgctac aaccagatga aaggaatggg gatgcggtca |
| 301 | gggggccatg ctgggatggg gcccccgccc agccccatgg accagcactc ccaaggttac |
| 361 | ccctcgcccc tgggtggctc tgagcatgcc tctagtccag ttccagccag tggcccgtct |
| 421 | tcggggcccc agatgtcttc cgggccagga ggtgccccgc tggatggtgc tgacccccag |
| 481 | gccttggggc agcagaaccg gggcccaacc ccatttaacc agaaccagct gcaccagctc |
| 541 | agagctcaga tcatggccta caagatgctg gccagggggc agcccctccc cgaccacctg |
| 601 | cagatggcgg tgcagggcaa gcggccgatg cccgggatgc agcagcagat gccaacgcta |
| 661 | cctccaccct cggtgtccgc aacaggaccc ggccctggcc ctggccctgg ccccggcccg |
| 721 | ggtcccggcc cggcacctcc aaattacagc aggcctcatg gtatgggagg gcccaacatg |
| 781 | cctcccccag gaccctcggg cgtgcccccc gggatgccag gccagcctcc tggagggcct |
| 841 | cccaagccct ggcctgaagg acccatggcg aatgctgctg cccccacgag cacccctcag |
| 901 | aagctgattc ccccgcagcc aacgggccgc ccttcccccg cgccccctgc cgtcccaccc |
| 961 | gccgcctcgc ccgtgatgcc accgcagacc cagtcccccg ggcagccggc ccagcccgcg |
| 1021 | cccatggtgc cactgcacca gaagcagagc cgcatcaccc ccatccagaa gccgcggggc |
| 1081 | ctcgaccctg tggagatcct gcaggagcgc gagtacaggc tgcaggctcg catcgcacac |
| 1141 | cgaattcagg aacttgaaaa ccttcccggg tccctggccg gggatttgcg aaccaaagcg |
| 1201 | accattgagc tcaaggccct caggctgctg aacttccaga ggcagctgcg ccaggaggtg |
| 1261 | gtggtgtgca tgcggaggga cacagcgctg gagacagccc tcaatgctaa ggcctacaag |
| 1321 | cgcagcaagc gccagtccct gcgcgaggcc cgcatcactg agaagctgga gaagcagcag |
| 1381 | aagatcgagc aggagcgcaa gcgccggcag aagcaccagg aatacctcaa tagcattctc |
| 1441 | cagcatgcca aggatttcaa ggaatatcac agatccgtca caggcaaaat ccagaagctg |
| 1501 | accaaggcag tggccacgta ccatgccaac acggagcggg agcagaagaa agagaacgag |
| 1561 | cggatcgaga aggagcgcat gcggaggctc atggctgaag atgaggaggg gtaccgcaag |
| 1621 | ctcatcgacc agaagaagga caagcgcctg gcctacctct tgcagcagac agacgagtac |
| 1681 | gtggctaacc tcacggagct ggtgcggcag cacaaggctg cccaggtcgc caaggagaaa |
| 1741 | aagaagaaaa agaaaaagaa gaaggcagaa aatgcagaag gacagacgcc tgccattggg |
| 1801 | ccggatggcg agcctctgga cgagaccagc cagatgagcg acctcccggt gaaggtgatc |
| 1861 | cacgtggaga gtgggaagat cctcacaggc acagatgccc ccaaagccgg gcagctggag |
| 1921 | gcctggctcg agatgaaccc ggggtatgaa gtagctccga ggtctgatag tgaagaaagt |
| 1981 | ggctcagaag aagaggaaga ggaggaggag gaagagcagc cgcaggcagc acagcctccc |
| 2041 | accctgcccg tggaggagaa gaagaagatt ccagatccag acagcgatga cgtctctgag |
| 2101 | gtggacgcgc ggcacatcat tgagaatgcc aagcaagatg tcgatgatga atatggcgtg |
| 2161 | tcccaggccc ttgcacgtgg cctgcagtcc tactatgccg tggcccatgc tgtcactgag |
| 2221 | agagtggaca agcagtcagc gcttatggtc aatggtgtcc tcaaacagta ccagatcaaa |
| 2281 | ggtttggagt ggctggtgtc cctgtacaac aacaacctga acggcatcct ggccgacgag |
| 2341 | atgggcctgg ggaagaccat ccagaccatc gcgctcatca cgtacctcat ggagcacaaa |
| 2401 | cgcatcaatg ggcccttcct catcatcgtg cctctctcaa cgctgtccaa ctgggcgtac |
| 2461 | gagtttgaca agtgggcccc ctccgtggtg aaggtgtctt acaagggatc cccagcagca |
| 2521 | agacgggcct ttgtccccca gctccggagt gggaagttca acgtcttgct gacgacgtac |
| 2581 | gagtacatca tcaaagacaa gcacatcctc gccaagatcc gttggaagta catgattgtg |
| 2641 | gacgaaggtc accgcatgaa gaaccaccac tgcaagctga cgcaggtgct caacacgcac |
| 2701 | tatgtggcac cccgccgcct gctgctgacg ggcacaccgc tgcagaacaa gcttcccgag |
| 2761 | ctctgggcgc tgctcaactt cctgctgccc accatcttca agagctgcag caccttcgag |
| 2821 | cagtggttta acgcaccctt tgccatgacc ggggaaaagg tggacctgaa tgaggaggaa |
| 2881 | accattctca tcatccggcg tctccacaaa gtgctgcggc ccttcttgct ccgacgactc |
| 2941 | aagaaggaag tcgaggccca gttgcccgaa aaggtggagt acgtcatcaa gtgcgacatg |
| 3001 | tctgcgctgc agcgagtgct ctaccgccac atgcaggcca agggcgtgct gctgactgat |
| 3061 | ggctccgaga aggacaagaa gggcaaaggc ggcaccaaga ccctgatgaa caccatcatg |
| 3121 | cagctgcgga agatctgcaa ccacccctac atgttccagc acatcgagga gtccttttcc |
| 3181 | gagcacttgg ggttcactgg cggcattgtc caagggctgg acctgtaccg agcctcgggt |
| 3241 | aaatttgagc ttcttgatag aattcttccc aaactccgag caaccaacca caaagtgctg |
| 3301 | ctgttctgcc aaatgacctc cctcatgacc atcatggaag attactttgc gtatcgcggc |
| 3361 | tttaaatacc tcaggcttga tggaaccacg aaggCggagg accggggcat gctgctgaaa |
| 3421 | accttcaacg agcccggctc tgagtacttc atcttcctgc tcagcacccg ggctgggggg |
| 3481 | ctcggcctga acctccagtc ggcagacact gtgatcattt ttgacagcga ctggaatcct |
| 3541 | caccaggacc tgcaagcgca ggaccgagcc caccgcatcg ggcagcagaa cgaggtgcgt |
| 3601 | gtgctccgcc tctgcaccgt caacagcgtg gaggagaaga tcctagctgc agccaagtac |
| 3661 | aagctcaacg tggaccagaa ggtgatccag gccggcatgt tcgaccagaa gtcctccagc |
| 3721 | catgagcggc gcgccttcct gcaggccatc ctggagcacg aggagcagga tgaggaggaa |
| 3781 | gacgaggtgc ccgacgacga gaccgtcaac cagatgatcg cccggcacga ggaggagttt |
| 3841 | gatctgttca tgcgcatgga cctggaccgc aggcgcgagg aggcccgcaa ccccaagcgg |
| 3901 | aagccgcgcc tcatggagga ggacgagctc ccctcgtgga tcatcaagga cgacgcggag |
| 3961 | gtggagcggc tgacctgtga ggaggaggag gagaagatgt tcggccgtgg ctcccgccac |
| 4021 | cgcaaggagg tggactacag cgactcactg acggagaagc agtggctcaa ggccatcgag |
| 4081 | gagggcacgc tggaggagat cgaagaggag gtccggcaga agaaatcatc acggaagcgc |
| 4141 | aagcgagaca gcgacgccgg ctcctccacc ccgaccacca gcacccgcag ccgcgacaag |
| 4201 | gacgacgaga gcaagaagca gaagaagcgc gggCggccgc ctgccgagaa actctcccct |
| 4261 | aacccaccca acctcaccaa gaagatgaag aagattgtgg atgccgtgat caagtacaag |
| 4321 | gacagcagca gtggacgtca gctcagcgag gtcttcatcc agctgccctc gcgaaaggag |
| 4381 | ctgcccgagt actacgagct catccgcaag cccgtggact tcaagaagat aaaggagcgc |
| 4441 | attcgcaacc acaagtaccg cagcctcaac gacctagaga aggacgtcat gctcctgtgc |
| 4501 | cagaacgcac agaccttcaa cctggagggc tccctgatct atgaagactc catcgtcttg |
| 4561 | cagtcggtct tcaccagcgt gcggcagaaa atcgagaagg aggatgacag tgaaggcgag |
| 4621 | gagagtgagg aggaggaaga gggcgaggag gaaggctccg aatccgaatc tcggtccgtc |
| 4681 | aaagtgaaga tcaagcttgg ccggaaggag aaggcacagg accggctgaa gggcggccgg |
| 4741 | cggcggccga gccgagggtc ccgagccaag ccggtcgtga gtgacgatga cagtgaggag |
| 4801 | gaacaagagg aggaccgctc aggaagtggc agcgaagaag actgagcccc gacattccag |
| 4861 | tctcgacccc gagcccctcg ttccagagct gagatggcat aggccttagc agtaacgggt |
| 4921 | agcagcagat gtagtttcag acttggagta aaactgtata aacaaaagaa tcttccatat |
| 4981 | ttatacagca gagaagctgt aggactgttt gtgactggcc ctgtcctggc atcagtagca |
| 5041 | tctgtaacag cattaactgt cttaaagaga gagagagaga attccgaatt ggggaacaca |
| 5101 | cgatacctgt ttttcttttc cgttgctggc agtactgttg cgccgcagtt tggagtcact |
| 5161 | gtagttaagt gtggatgcat gtgcgtcacc gtccactcct cctactgtat tttattggac |
| 5221 | aggtcagact cgccgggggc ccggcgaggg tatgtcagtg tcactggatg tcaaacagta |
| 5281 | ataaattaaa ccaacaacaa aacgcacagc caaaaaaaaa |

SEQ ID NO: 202 Human SMARCA4 Amino Acid Sequence Isoform F
(NP_001122320.1)

| | |
|---|---|
| 1 | mstpdpplgg tprpgpspgp gpspgamlgp spgpspgsah smmgpspgpp saghpiptqg |
| 61 | pggypqdnmh qmhkpmesmh ekgmsddpry nqmkgmgmrs gghagmgppp spmdqhsqgy |
| 121 | psplggseha sspvpasgps sgpqmssgpg gapldgadpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa pmvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfkeyh rsvtgkiqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqaaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlkaie egtleeieee vrqkkssrkr |
| 1381 | krdsdagsst pttstrsrdk ddeskkqkkr grppaeklsp nppnltkkmk kivdavikyk |
| 1441 | dssgrqlsev fiqlpsrkel peyyelirkp vdfkkikeri rnhkyrsind lekdvmllcq |
| 1501 | naqtfnlegs liyedsivlq svftsvrqki ekeddsegee seeeeegeee gsesesrsvk |
| 1561 | vkiklgrkek aqdrlkggrr rpsrgsrakp vvsdddseee qeedrsgsgs eed |

SEQ ID NO: 203 Human SMARCA4 cDNA Sequence Variant 7 (NM_001128848.1,
CDS: 1-4842)

| | |
|---|---|
| 1 | atgtccactc cagacccacc cctgggcgga actcctcggc caggtccttc cccgggccct |
| 61 | ggcccttccc ctggagccat gctgggccct agcccgggtc cctcgccggg ctccgcccac |
| 121 | agcatgatgg ggcccagccc agggccgccc tcagcaggac accccatccc cacccagggg |
| 181 | cctggagggt accctcagga caacatgcac cagatgcaca agcccatgga gtccatgcat |
| 241 | gagaagggca tgtcggacga cccgcgctac aaccagatga aaggaatggg gatgcggtca |
| 301 | gggggccatg ctgggatggg gcccccgccc agccccatgg accagcactc ccaaggttac |
| 361 | ccctcgcccc tgggtggctc tgagcatgcc tctagtccag ttccagccag tggcccgtct |
| 421 | tcggggcccc agatgtcttc cgggccagga ggtgccccgc tggatggtgc tgacccccag |
| 481 | gccttggggc agcagaaccg gggcccaacc ccatttaacc agaaccagct gcaccagctc |
| 541 | agagctcaga tcatggccta caagatgctg gccagggggc agcccctccc cgaccacctg |
| 601 | cagatggcgg tgcagggcaa gcggccgatg cccgggatgc agcagcagat gccaacgcta |
| 661 | cctccaccct cggtgtccgc aacaggaccc ggccctggcc ctggccctgg ccccggcccg |
| 721 | ggtcccggcc cggcacctcc aaattacagc aggcctcatg gtatgggagg gcccaacatg |
| 781 | cctcccccag gaccctcggg cgtgcccccc gggatgccag gccagcctcc tggagggcct |
| 841 | cccaagccct ggcctgaagg acccatggcg aatgctgctg cccccacgag cacccctcag |
| 901 | aagctgattc ccccgcagcc aacgggccgc ccttcccccg cgccccctgc cgtcccaccc |
| 961 | gccgcctcgc ccgtgatgcc accgcagacc cagtcccccg ggcagccggc ccagcccgcg |
| 1021 | cccatggtgc cactgcacca gaagcagagc cgcatcaccc ccatccagaa gccgcggggc |
| 1081 | ctcgaccctg tggagatcct gcaggagcgc gagtacaggc tgcaggctcg catcgcacac |
| 1141 | cgaattcagg aacttgaaaa ccttcccggg tccctggccg gggatttgcg aaccaaagcg |
| 1201 | accattgagc tcaaggccct caggctgctg aacttccaga ggcagctgcg ccaggaggtg |
| 1261 | gtggtgtgca tgcggaggga cacagcgctg gagacagccc tcaatgctaa ggcctacaag |
| 1321 | cgcagcaagc gccagtccct gcgcgaggcc cgcatcactg agaagctgga gaagcagcag |
| 1381 | aagatcgagc aggagcgcaa gcgccggcag aagcaccagg aatacctcaa tagcattctc |
| 1441 | cagcatgcca aggatttcaa ggaatatcac agatccgtca caggcaaaat ccagaagctg |
| 1501 | accaaggcag tggccacgta ccatgccaac acggagcggg agcagaagaa agagaacgag |
| 1561 | cggatcgaga aggagcgcat gcggaggctc atggctgaag atgaggaggg gtaccgcaag |
| 1621 | ctcatcgacc agaagaagga caagcgcctg gcctacctct tgcagcagac agacgagtac |
| 1681 | gtggctaacc tcacggagct ggtgcggcag cacaaggctg cccaggtcgc caaggagaaa |
| 1741 | aagaagaaaa agaaaaagaa gaaggcagaa aatgcagaag gacagacgcc tgccattggg |
| 1801 | ccggatggcg agcctctgga cgagaccagc cagatgagcg acctcccggt gaaggtgatc |
| 1861 | cacgtggaga gtgggaagat cctcacaggc acagatgccc ccaaagccgg gcagctggag |
| 1921 | gcctggctcg agatgaaccc ggggtatgaa gtagctccga ggtctgatag tgaagaaagt |
| 1981 | ggctcagaag aagaggaaga ggaggaggag gaagagcagc cgcaggcagc acagcctccc |
| 2041 | accctgcccg tggaggagaa gaagaagatt ccagatccag acagcgatga cgtctctgag |
| 2101 | gtggacgcgc ggcacatcat tgagaatgcc aagcaagatg tcgatgatga atatggcgtg |
| 2161 | tcccaggccc ttgcacgtgg cctgcagtcc tactatgccg tggcccatgc tgtcactgag |
| 2221 | agagtggaca agcagtcagc gcttatggtc aatggtgtcc tcaaacagta ccagatcaaa |
| 2281 | ggtttggagt ggctggtgtc cctgtacaac aacaacctga acggcatcct ggccgacgag |
| 2341 | atgggcctgg ggaagaccat ccagaccatc gcgctcatca cgtacctcat ggagcacaaa |
| 2401 | cgcatcaatg ggcccttcct catcatcgtg cctctctcaa cgctgtccaa ctgggcgtac |
| 2461 | gagtttgaca agtgggcccc ctccgtggtg aaggtgtctt acaagggatc cccagcagca |
| 2521 | agacgggcct ttgtccccca gctccggagt gggaagttca acgtcttgct gacgacgtac |
| 2581 | gagtacatca tcaaagacaa gcacatcctc gccaagatcc gttggaagta catgattgtg |
| 2641 | gacgaaggtc accgcatgaa gaaccaccac tgcaagctga cgcaggtgct caacacgcac |
| 2701 | tatgtggcac cccgccgcct gctgctgacg ggcacaccgc tgcagaacaa gcttcccgag |
| 2761 | ctctgggcgc tgctcaactt cctgctgccc accatcttca agagctgcag caccttcgag |
| 2821 | cagtggttta acgcaccctt tgccatgacc ggggaaaagg tggacctgaa tgaggaggaa |
| 2881 | accattctca tcatccggcg tctccacaaa gtgctgcggc ccttcttgct ccgacgactc |
| 2941 | aagaaggaag tcgaggccca gttgcccgaa aaggtggagt acgtcatcaa gtgcgacatg |
| 3001 | tctgcgctgc agcgagtgct ctaccgccac atgcaggcca agggcgtgct gctgactgat |
| 3061 | ggctccgaga aggacaagaa gggcaaaggC ggcaccaaga ccctgatgaa caccatcatg |
| 3121 | cagctgcgga agatctgcaa ccacccctac atgttccagc acatcgagga gtccttttcc |
| 3181 | gagcacttgg ggttcactgg cggcattgtc caagggctgg acctgtaccg agcctcgggt |
| 3241 | aaatttgagc ttcttgatag aattcttccc aaactccgag caaccaacca caaagtgctg |
| 3301 | ctgttctgcc aaatgacctc cctcatgacc atcatggaag attactttgc gtatcgcggc |
| 3361 | tttaaatacc tcaggcttga tggaaccacg aaggcggagg accggggcat gctgctgaaa |
| 3421 | accttcaacg agcccggctc tgagtacttc atcttcctgc tcagcacccg ggctgggggg |
| 3481 | ctcggcctga acctccagtc ggcagacact gtgatcattt ttgacagcga ctggaatcct |
| 3541 | caccaggacc tgcaagcgca ggaccgagcc caccgcatcg ggcagcagaa cgaggtgcgt |
| 3601 | gtgctccgcc tctgcaccgt caacagcgtg gaggagaaga tcctagctgc agccaagtac |
| 3661 | aagctcaacg tggaccagaa ggtgatccag gccggcatgt tcgaccagaa gtcctccagc |
| 3721 | catgagcggc gcgccttcct gcaggccatc ctggagcacg aggagcagga tgaggaggaa |
| 3781 | gacgaggtgc ccgacgacga gaccgtcaac cagatgatcg cccggcacga ggaggagttt |
| 3841 | gatctgttca tgcgcatgga cctggaccgc aggcgcgagg aggcccgcaa ccccaagcgg |
| 3901 | aagccgcgcc tcatggagga ggacgagctc ccctcgtgga tcatcaagga cgacgcggag |
| 3961 | gtggagcggc tgacctgtga ggaggaggag gagaagatgt tcggccgtgg ctcccgccac |
| 4021 | cgcaaggagg Tggactacag cgactcactg acggagaagc agtggctcaa ggccatcgag |
| 4081 | gagggcacgc tggaggagat cgaagaggag gtccggcaga agaaatcatc acggaagcgc |
| 4141 | aagcgagaca gcgacgccgg ctcctccacc ccgaccacca gcacccgcag ccgcgacaag |
| 4201 | gacgacgaga gcaagaagca gaagaagcgc gggcggccgc ctgccgagaa actctcccct |
| 4261 | aacccaccca acctcaccaa gaagatgaag aagattgtgg atgccgtgat caagtacaag |
| 4321 | gacagcagtg gacgtcagct cagcgaggtc ttcatccagc tgccctcgcg aaaggagctg |
| 4381 | cccgagtact acgagctcat ccgcaagccc gtggacttca agaagataaa ggagcgcatt |
| 4441 | cgcaaccaca agtaccgcag cctcaacgac ctagagaagg acgtcatgct cctgtgccag |
| 4501 | aacgcacaga ccttcaacct ggagggctcc ctgatctatg aagactccat cgtcttgcag |
| 4561 | tcggtcttca ccagcgtgcg gcagaaaatc gagaaggagg atgacagtga aggcgaggag |
| 4621 | agtgaggagg aggaagaggg cgaggaggaa ggctccgaat ccgaatctcg gtccgtcaaa |
| 4681 | gtgaagatca agcttggccg gaaggagaag gcacaggacc ggctgaaggg cggccggcgg |
| 4741 | cggccgagcc gagggtcccg agccaagccg gtcgtgagtg acgatgacag tgaggaggaa |
| 4801 | caagaggagg accgctcagg aagtggcagc gaagaagact gagccccgac attccagtct |
| 4861 | cgaccccgag cccctcgttc cagagctgag atggcatagg ccttagcagt aacgggtagc |
| 4921 | agcagatgta gtttcagact tggagtaaaa ctgtataaac aaaagaatct tccatattta |
| 4981 | tacagcagag aagctgtagg actgtttgtg actggccctg tcctggcatc agtagcatct |
| 5041 | gtaacagcat taactgtctt aaagagagag agagagaatt ccgaattggg gaacacacga |
| 5101 | tacctgtttt tcttttccgt tgctggcagt actgttgcgc cgcagtttgg agtcactgta |
| 5161 | gttaagtgtg gatgcatgtg cgtcaccgtc cactcctcct actgtatttt attggacagg |
| 5221 | tcagactcgc cgggggcccg gcgagggtat gtcagtgtca ctggatgtca aacagtaata |
| 5281 | aattaaacca acaacaaaac gcacagccaa aaaaaaa |

SEQ ID NO: 204 Mouse SMARCA4 cDNA Sequence variant 1 (NM_001174078.1;
CDS: 261-5114)

| | |
|---|---|
| 1 | ggcaagtgga gcgggtagac agggaggcgg gggcgcgcgg cgggcgcgtg cggtgggggg |
| 61 | gggtggcctg gcgaagccca gcgggcgcgc gcgcgaggct ttcccactcg cttggcagcg |
| 121 | gcggagacgg cttctttgtt tcctgaggag aagcgagacg cccactctgt ccccgacccc |
| 181 | tcgtggaggg ttgggggcgg cgccaggaag gttacggcgc cgttacctcc aggagaccag |
| 241 | tgcctgtagc tccagtaaag atgtctactc cagacccacc cttgggtggg actcctcggc |
| 301 | ctggtccttc cccaggccct ggtccttcac ctggtgcaat gctgggtcct agccctggcc |
| 361 | cctcaccagg ttctgcccac agcatgatgg ggccaagccc aggacctcct tcagcaggac |
| 421 | atcccatgcc cacccagggg cctggagggt acccccagga caacatgcat cagatgcaca |
| 481 | agcctatgga gtccatgcac gagaagggca tgcctgatga cccacgatac aaccagatga |
| 541 | aagggatggg catgcggtca ggggcccaca caggcatggc acctccacct agtcccatgg |
| 601 | accagcattc tcaaggttac ccctcacccc tcggcggctc tgaacatgcc tccagtcctg |
| 661 | tcccagccag tggcccatct tcaggccccc agatgtcctc tgggccagga ggggccccac |
| 721 | tagatggttc tgatccccag gccttgggac agcaaaacag aggcccaacc ccatttaacc |
| 781 | agaaccagct gcatcaactc agagctcaga taatggccta caagatgttg gccaggggcc |
| 841 | agccattgcc cgaccacctg cagatggccg tgcaaggcaa gcggccgatg cctggaatgc |
| 901 | agcaacagat gccaacacta cctccaccct cagtgtccgc cacaggaccc ggacctggac |
| 961 | ccggccctgg ccctggccct ggcccaggac cagcccctcc aaattacagt agaccccatg |
| 1021 | gtatgggagg gcccaacatg cctcccccag gaccctcagg tgtgcccccc gggatgcctg |
| 1081 | gtcagccgcc tggagggcct cccaagccat ggcctgaagg acccatggcc aatgctgctg |
| 1141 | cccccacaag caccccacag aagctgattc ctccgcaacc aacaggccgt ccttcacctg |
| 1201 | cacctcctgc tgtcccgcct gctgcctcac ctgtaatgcc accacaaaca cagtccccag |
| 1261 | ggcagccagc ccagcctgct ccattggtgc cactgcacca gaagcagagc cgaatcaccc |
| 1321 | ccatccagaa gccccgaggc cttgaccctg tggagatcct acaagagcgg gagtacaggc |
| 1381 | ttcaggctcg aatcgcacac agaattcagg aacttgaaaa cctccctggg tccctggctg |
| 1441 | gggaccttcg aaccaaagca accatcgaac tcaaggccct taggttgctg aacttccaga |
| 1501 | ggcagctgcg ccaggaggtg gtggtgtgca tgcgaagaga cacagccctg gagacagccc |
| 1561 | tcaatgccaa ggcctacaag cgcagcaaac gtcagtcact acgggaggcc cgcatcactg |
| 1621 | agaagttgga gaagcagcag aagattgaac aggagcgcaa gcgccgccag aagcaccagg |
| 1681 | agtacctcaa cagcattctg cagcatgcca aggacttcag ggagtatcac agatcagtca |
| 1741 | caggcaaact ccagaaactc accaaggctg tggccaccta ccatgccaac actgagcggg |
| 1801 | agcagaagaa agaaaatgag cgcattgaga aggagcgaat gcggaggctt atggctgaag |
| 1861 | atgaggaggg ctaccgcaaa ctcattgacc agaagaagga caagcgcctg gcctaccttc |
| 1921 | tgcagcagac agatgagtat gtggccaacc tcacagagct ggtgcggcag cacaaagctg |
| 1981 | cccaggttgc caaggagaag aagaagaaaa agaaaaagaa gaaggcagaa aatgctgaag |
| 2041 | gacagacacc tgctattgga ccagatggtg agcctctgga tgagaccagc cagatgagtg |
| 2101 | acctccctgt gaaggtgatc cacgtggaga gtggcaagat cctcactggc acagatgccc |
| 2161 | caaaagccgg gcagctggaa gcctggcttg aaatgaaccc agggtatgaa gtagccccca |
| 2221 | ggtcagacag tgaagaaagt ggctctgaag aggaggagga ggaggaggaa gaggagcagc |
| 2281 | ctcagcccgc acagccccct acactgcctg tggaagaaaa gaagaagatt ccagacccag |
| 2341 | acagcgatga tgtctctgag gtggacgccc gacacattat tgagaacgcc aagcaagatg |
| 2401 | tggacgatga gtacggtgtg tcccaggccc ttgctcgtgg cctgcagtct tactatgctg |
| 2461 | tggcccatgc agtcacagag agagtagata agcagtccgc cctcatggtc aacggtgtcc |
| 2521 | tcaaacagta ccagatcaag ggtttggagt ggctggtgtc cctgtacaac aacaacctga |
| 2581 | atggcatcct ggctgatgag atggggctgg ggaagaccat ccagaccatc gcgctcatca |
| 2641 | catacctcat ggagcacaag cgcatcaacg ggcctttcct catcatcgtg cctctctcga |
| 2701 | cactgtcaaa ctgggcgtat gaatttgaca agtgggcccc ctctgtggtg aaggtttctt |
| 2761 | acaagggctc tccagctgca aggcgagctt ttgtcccaca gcttcgcagt gggaagttca |
| 2821 | acgtcttact gaccacctat gaatatatca tcaaagacaa gcatatccta gccaagatcc |
| 2881 | gctggaagta catgattgtg gatgaaggcc accgcatgaa aaaccaccac tgcaagttga |
| 2941 | cgcaggtcct taacacacac tacgtggccc ctcggcgcct gcttcttaca ggcacaccac |
| 3001 | tgcagaacaa gctaccggag ctctgggccc tgcttaactt cctgctcccc actatcttca |
| 3061 | agagctgcag caccttcgaa cagtggttca atgcaccctt tgccatgact ggagaaaagg |
| 3121 | tggacctgaa tgaagaggag actatcctca ttattcgtcg cctacacaaa gttctgcggc |
| 3181 | ccttcctgct gcggcggctc aagaaggaag ttgaagccca gctccctgag aaggtagagt |
| 3241 | atgtcatcaa atgcgacatg tcagccctgc agcgtgtgct gtaccgtcac atgcaggcca |
| 3301 | aaggtgtgct gctgactgac ggctccgaga aggacaagaa gggcaaaggt ggcaccaaga |
| 3361 | cactgatgaa cactattatg caactgcgta agatctgcaa ccacccctac atgttccagc |
| 3421 | acatcgagga gtccttttct gagcacttgg ggttcaccgg cggcatcgtg caaggattgg |
| 3481 | acctttaccg tgcctcaggg aaatttgaac ttcttgatag aattctaccc aaactccgtg |
| 3541 | caacgaacca taaagtgctc ctcttttgcc aaatgacctc cctcatgacc atcatggaag |
| 3601 | actactttgc ataccgtggc ttcaaatacc tcaggcttga tggaaccaca aaagcagaag |
| 3661 | accggggcat gctgttgaaa acctttaatg aacctggctc tgagtatttc attttcctgc |
| 3721 | tcagtacccg tgctgggggg ctgggcctga atctgcagtc agctgacact gtgatcatct |
| 3781 | ttgacagtga ctggaatccc caccaggacc tgcaagcaca ggatcgagcc catcgcattg |
| 3841 | gacagcagaa tgaggtgcgt gttcttcgcc tgtgcacggt caacagtgtg gaagagaaga |
| 3901 | tactggctgc tgccaaatac aaactcaatg tggatcagaa ggtgatccag gcaggcatgt |
| 3961 | tcgaccagaa gtcgtccagc catgagaggc gtgccttcct gcaggccatc ctggagcacg |
| 4021 | aggagcagga tgaggaggaa gatgaggtgc ctgatgatga gaccgtcaac cagatgattg |
| 4081 | cccggcacga agaagagttt gacctcttca tgcgcatgga cttggaccgc cggcgtgaag |
| 4141 | aagcccgcaa ccccaagcgg aagccacgcc tgatggaaga ggatgagctc ccatcctgga |
| 4201 | tcatcaagga tgatgccgag gtggagcggc tgacatgtga agaggaagag gagaagatgt |
| 4261 | tcggccgtgg ttctcgccac cgcaaggagg tagactacag cgactcactg acagagaagc |
| 4321 | agtggctcaa gaccctgaag gctatcgagg agggcacgct ggaggagatc gaagaggagg |
| 4381 | tccggcagaa gaaatcttca cgtaagcgta agcgagacag cgaggccggc tcctccaccc |
| 4441 | cgaccaccag cacccgcagc cgtgacaagg atgaggagag caagaagcag aagaaacgtg |
| 4501 | ggcggccacc tgctgagaag ctgtccccaa acccacctaa cctcaccaag aagatgaaga |
| 4561 | agatcgtgga tgctgtgatc aagtacaaag acagcagcag tggacgtcag ctcagcgagg |
| 4621 | tgttcatcca gctcccctct cgcaaggagc ttcctgagta ctatgagctc atccgaaagc |
| 4681 | ctgtggactt caagaagatc aaggaacgca tccgaaacca caagtaccgc agcctcaatg |
| 4741 | acctggagaa ggatgtgatg ctgctgtgcc agaacgctca gacgttcaac ctcgagggtt |
| 4801 | ccctgatcta tgaggactcc atcgtcctgc agtctgtctt caccagcgta cggcagaaga |
| 4861 | ttgagaagga ggacgacagt gaaggcgagg aaagcgagga ggaggaggag ggcgaggagg |
| 4921 | aaggctccga gtctgagtcc cgctccgtca aggtgaagat caagctgggc cgcaaggaga |
| 4981 | aggcccagga ccgactcaag gggggccgcc ggcggccaag ccggggatcc cgggccaagc |
| 5041 | cggttgtgag tgacgatgac agtgaggagg agcaggagga ggaccgctca ggaagtggca |
| 5101 | gtgaggaaga ctgaaccaga cattcctgag tcctgacccc gaggcgctcg tcccagccaa |
| 5161 | gatggagtag cccttagcag tgatgggtag caccagatgt agtttcgaac ttggagaact |
| 5221 | gtacacatgc aatcttccac atttttaggc agagaagtat aggcctgtct gtcggccctg |
| 5281 | gcctggcctc gagtctctac cagcattaac tgtctagaga ggggacctcc tgggagcacc |
| 5341 | atccacctcc ccaggcccca gtcactgtag ctcagtggat gcatgcgcgt gccggccgct |
| 5401 | ccttgtactg tatcttactg gacagggcca gctctccagg aggctcacag gcccagcggg |
| 5461 | tatgtcagtg tcactggagt cagacagtaa taaattaaag caatgacaag ccaccactgg |
| 5521 | ctccctggac tccttgctgt cagcagtggc tccggggcca cagagaagaa agaaagactt |
| 5581 | ttaggaactg ggtctaactt atgggcaaag tacttgcctt gccaggtgta tgggttttgc |
| 5641 | attcccatca cccacacacc ctaaacaagc caagtcagtg agcttcaagt tagagcctcc |
| 5701 | acctcaatgt gtacgtggaa agcaatcaaa gatgatgcct agcatccacc tctggccctc |
| 5761 | atgtgcagat gtacacacac tgaattacat acacgggaca cacacatcca cacggaggca |
| 5821 | gtccatgact tgcactgggg agatggtacc ataggcgaaa gtgccacagg cacagggcca |
| 5881 | ggctaattta gtcctgcagt cctgtgctct taagatgaag gcacaaagag gaaccccagg |
| 5941 | cgctccaact agcatgccag gcagtgacaa gaccctgctt caaatgaatc agagcccaca |
| 6001 | ttcagtattg ccctcttacc cgatgcgatg cccatgccct cacatatgaa tgtgtatata |
| 6061 | tacatacata cgtaaaataa ttctttttta aattatagac atttttgtgt gaatgttttg |
| 6121 | cctgaatgtg tgtgtgtgtg tgtgtgtgtg tgtgtgtgtg tgtgtgtgtg tatcaagtac |
| 6181 | attcctagag cctacagagg tcaagggagg gcattggatc tggaactgga gtcacatgag |
| 6241 | gctgtgagca actgtgtggg ttcctgggcc tttgcaacag cagttagtac tcttcaccac |
| 6301 | tgagccattt ctccaatctc aaaaagaagc attcttttaa atgaagactg aaataaataa |
| 6361 | gtaggacttg ccttgg |

SEQ ID NO: 205 Mouse SMARCA4 Amino Acid Sequence isoform 1
(NP_001167549.1)

| | |
|---|---|
| 1 | mstpdpplgg tprpgpspgp gpspgamlgp spgpspgsah smmgpspgpp saghpmptqg |
| 61 | pggypqdnmh qmhkpmesmh ekgmpddpry nqmkgmgmrs gahtgmappp spmdqhsqgy |
| 121 | psplggseha sspvpasgps sgpqmssgpg gapldgsdpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa plvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfreyh rsvtgklqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqpaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprilllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlktlk aieegtleei eeevrqkkss |
| 1381 | rkrkrdseag sstpttstrs rdkdeeskkq kkrgrppaek lspnppnltk kmkkivdavi |
| 1441 | kykdsssgrq lsevfiqlps rkelpeyyel irkpvdfkki kerirnhkyr slndlekdvm |
| 1501 | llcqnaqtfn legsliyeds ivlqsvftsv rqkiekedds egeeseeeee geeegseses |
| 1561 | rsvkvkiklg rkekagdrlk ggrrrpsrgs rakpvvsddd seeeqeedrs gsgseed |

SEQ ID NO: 206 Mouse SMARCA4 cDNA Sequence variant 2 (NM_011417.3)

| | |
|---|---|
| 1 | ggcaagtgga gcgggtagac agggaggcgg gggcgcgcgg cgggcgcgtg cggtgggggg |
| 61 | gggtggcctg gcgaagccca gcgggcgcgc gcgcgaggct ttcccactcg cttggcagcg |
| 121 | gcggagacgg cttctttgtt tcctgaggag aagcgagacg cccactctgt ccccgacccc |
| 181 | tcgtggaggg ttgggggcgg cgccaggaag gttacggcgc cgttacctcc aggagaccag |
| 241 | tgcctgtagc tccagtaaag atgtctactc cagacccacc cttgggtggg actcctcggc |
| 301 | ctggtccttc cccaggccct ggtccttcac ctggtgcaat gctgggtcct agccctggcc |
| 361 | cctcaccagg ttctgcccac agcatgatgg ggccaagccc aggacctcct tcagcaggac |
| 421 | atcccatgcc cacccagggg cctggagggt acccccagga caacatgcat cagatgcaca |
| 481 | agcctatgga gtccatgcac gagaagggca tgcctgatga cccacgatac aaccagatga |
| 541 | aagggatggg catgcggtca ggggcccaca caggcatggc acctccacct agtcccatgg |
| 601 | accagcattc tcaaggttac ccctcacccc tcggcggctc tgaacatgcc tccagtcctg |
| 661 | tcccagccag tggcccatct tcaggccccc agatgtcctc tgggccagga ggggccccac |
| 721 | tagatggttc tgatccccag gccttgggac agcaaaacag aggcccaacc ccatttaacc |
| 781 | agaaccagct gcatcaactc agagctcaga taatggccta caagatgttg gccaggggcc |
| 841 | agccattgcc cgaccacctg cagatggccg tgcaaggcaa gcggccgatg cctggaatgc |
| 901 | agcaacagat gccaacacta cctccaccct cagtgtccgc cacaggaccc ggacctggac |
| 961 | ccggccctgg ccctggccct ggcccaggac cagcccctcc aaattacagt agaccccatg |
| 1021 | gtatgggagg gcccaacatg cctcccccag gaccctcagg tgtgcccccc gggatgcctg |
| 1081 | gtcagccgcc tggagggcct cccaagccat ggcctgaagg acccatggcc aatgctgctg |
| 1141 | cccccacaag caccccacag aagctgattc ctccgcaacc aacaggccgt ccttcacctg |
| 1201 | cacctcctgc tgtcccgcct gctgcctcac ctgtaatgcc accacaaaca cagtccccag |
| 1261 | ggcagccagc ccagcctgct ccattggtgc cactgcacca gaagcagagc cgaatcaccc |
| 1321 | ccatccagaa gccccgaggc cttgaccctg tggagatcct acaagagcgg gagtacaggc |
| 1381 | ttcaggctcg aatcgcacac agaattcagg aacttgaaaa cctccctggg tccctggctg |
| 1441 | gggaccttcg aaccaaagca accatcgaac tcaaggccct taggttgctg aacttccaga |
| 1501 | ggcagctgcg ccaggaggtg gtggtgtgca tgcgaagaga cacagccctg gagacagccc |
| 1561 | tcaatgccaa ggcctacaag cgcagcaaac gtcagtcact acgggaggcc cgcatcactg |
| 1621 | agaagttgga gaagcagcag aagattgaac aggagcgcaa gcgccgccag aagcaccagg |
| 1681 | agtacctcaa cagcattctg cagcatgcca aggacttcag ggagtatcac agatcagtca |
| 1741 | caggcaaact ccagaaactc accaaggctg tggccaccta ccatgccaac actgagcggg |
| 1801 | agcagaagaa agaaaatgag cgcattgaga aggagcgaat gcggaggctt atggctgaag |
| 1861 | atgaggaggg ctaccgcaaa ctcattgacc agaagaagga caagcgcctg gcctaccttc |
| 1921 | tgcagcagac agatgagtat gtggccaacc tcacagagct ggtgcggcag cacaaagctg |
| 1981 | cccaggttgc caaggagaag aagaagaaaa agaaaaagaa gaaggcagaa aatgctgaag |
| 2041 | gacagacacc tgctattgga ccagatggtg agcctctgga tgagaccagc cagatgagtg |
| 2101 | acctccctgt gaaggtgatc cacgtggaga gtggcaagat cctcactggc acagatgccc |
| 2161 | caaaagccgg gcagctggaa gcctggcttg aaatgaaccc agggtatgaa gtagccccca |
| 2221 | ggtcagacag tgaagaaagt ggctctgaag aggaggagga ggaggaggaa gaggagcagc |
| 2281 | ctcagcccgc acagccccct acactgcctg tggaagaaaa gaagaagatt ccagacccag |
| 2341 | acagcgatga tgtctctgag gtggacgccc gacacattat tgagaacgcc aagcaagatg |
| 2401 | tggacgatga gtacggtgtg tcccaggccc ttgctcgtgg cctgcagtct tactatgctg |
| 2461 | tggcccatgc agtcacagag agagtagata agcagtccgc cctcatggtc aacggtgtcc |
| 2521 | tcaaacagta ccagatcaag ggtttggagt ggctggtgtc cctgtacaac aacaacctga |
| 2581 | atggcatcct ggctgatgag atggggctgg ggaagaccat ccagaccatc gcgctcatca |
| 2641 | catacctcat ggagcacaag cgcatcaacg ggcctttcct catcatcgtg cctctctcga |
| 2701 | cactgtcaaa ctgggcgtat gaatttgaca agtgggcccc ctctgtggtg aaggtttctt |
| 2761 | acaagggctc tccagctgca aggcgagctt ttgtcccaca gcttcgcagt gggaagttca |
| 2821 | acgtcttact gaccacctat gaatatatca tcaaagacaa gcatatccta gccaagatcc |
| 2881 | gctggaagta catgattgtg gatgaaggcc accgcatgaa aaaccaccac tgcaagttga |
| 2941 | cgcaggtcct taacacacac tacgtggccc ctcggcgcct gcttcttaca ggcacaccac |
| 3001 | tgcagaacaa gctaccggag ctctgggccc tgcttaactt cctgctcccc actatcttca |
| 3061 | agagctgcag caccttcgaa cagtggttca atgcaccctt tgccatgact ggagaaaagg |
| 3121 | tggacctgaa tgaagaggag actatcctca ttattcgtcg cctacacaaa gttctgcggc |
| 3181 | ccttcctgct gcggcggctc aagaaggaag ttgaagccca gctccctgag aaggtagagt |
| 3241 | atgtcatcaa atgcgacatg tcagccctgc agcgtgtgct gtaccgtcac atgcaggcca |
| 3301 | aaggtgtgct gctgactgac ggctccgaga aggacaagaa gggcaaaggt ggcaccaaga |
| 3361 | cactgatgaa cactattatg caactgcgta agatctgcaa ccacccctac atgttccagc |
| 3421 | acatcgagga gtccttttct gagcacttgg ggttcaccgg cggcatcgtg caaggattgg |
| 3481 | acctttaccg tgcctcaggg aaatttgaac ttcttgatag aattctaccc aaactccgtg |
| 3541 | caacgaacca taaagtgctc ctcttttgcc aaatgacctc cctcatgacc atcatggaag |
| 3601 | actactttgc ataccgtggc ttcaaatacc tcaggcttga tggaaccaca aaagcagaag |
| 3661 | accggggcat gctgttgaaa acctttaatg aacctggctc tgagtatttc attttcctgc |
| 3721 | tcagtacccg tgctgggggg ctgggcctga atctgcagtc agctgacact gtgatcatct |
| 3781 | ttgacagtga ctggaatccc caccaggacc tgcaagcaca ggatcgagcc catcgcattg |
| 3841 | gacagcagaa tgaggtgcgt gttcttcgcc tgtgcacggt caacagtgtg gaagagaaga |
| 3901 | tactggctgc tgccaaatac aaactcaatg tggatcagaa ggtgatccag gcaggcatgt |
| 3961 | tcgaccagaa gtcgtccagc catgagaggc gtgccttcct gcaggccatc ctggagcacg |
| 4021 | aggagcagga tgaggaggaa gatgaggtgc ctgatgatga gaccgtcaac cagatgattg |
| 4081 | cccggcacga agaagagttt gacctcttca tgcgcatgga cttggaccgc cggcgtgaag |
| 4141 | aagcccgcaa ccccaagcgg aagccacgcc tgatggaaga ggatgagctc ccatcctgga |
| 4201 | tcatcaagga tgatgccgag gtggagcggc tgacatgtga agaggaagag gagaagatgt |
| 4261 | tcggccgtgg ttctcgccac cgcaaggagg tagactacag cgactcactg acagagaagc |
| 4321 | agtggctcaa ggctatcgag gagggcacgc tggaggagat cgaagaggag gtccggcaga |
| 4381 | agaaatcttc acgtaagcgt aagcgagaca gcgaggccgg ctcctccacc ccgaccacca |
| 4441 | gcacccgcag ccgtgacaag gatgaggaga gcaagaagca gaagaaacgt gggcggccac |
| 4501 | ctgctgagaa gctgtcccca aacccaccta acctcaccaa gaagatgaag aagatcgtgg |
| 4561 | atgctgtgat caagtacaaa gacagcagca gtggacgtca gctcagcgag gtgttcatcc |
| 4621 | agctcccctc tcgcaaggag cttcctgagt actatgagct catccgaaag cctgtggact |
| 4681 | tcaagaagat caaggaacgc atccgaaacc acaagtaccg cagcctcaat gacctggaga |
| 4741 | aggatgtgat gctgctgtgc cagaacgctc agacgttcaa cctcgagggt tccctgatct |
| 4801 | atgaggactc catcgtcctg cagtctgtct tcaccagcgt acggcagaag attgagaagg |
| 4861 | aggacgacag tgaaggcgag gaaagcgagg aggaggagga gggcgaggag gaaggctccg |
| 4921 | agtctgagtc ccgctccgtc aaggtgaaga tcaagctggg ccgcaaggag aaggcccagg |
| 4981 | accgactcaa ggggggccgc cggcggccaa gccggggatc ccgggccaag ccggttgtga |
| 5041 | gtgacgatga cagtgaggag gagcaggagg aggaccgctc aggaagtggc agtgaggaag |
| 5101 | actgaaccag acattcctga gtcctgaccc cgaggcgctc gtcccagcca agatggagta |
| 5161 | gcccttagca gtgatgggta gcaccagatg tagtttcgaa cttggagaac tgtacacatg |
| 5221 | caatcttcca catttttagg cagagaagta taggcctgtc tgtcggccct ggcctggcct |
| 5281 | cgagtctcta ccagcattaa ctgtctagag aggggacctc ctgggagcac catccacctc |
| 5341 | cccaggcccc agtcactgta gctcagtgga tgcatgcgcg tgccggccgc tccttgtact |
| 5401 | gtatcttact ggacagggcc agctctccag gaggctcaca ggcccagcgg gtatgtcagt |
| 5461 | gtcactggag tcagacagta ataaattaaa gcaatgacaa gccaccactg gctccctgga |
| 5521 | ctccttgctg tcagcagtgg ctccggggcc acagagaaga aagaaagact tttaggaact |
| 5581 | gggtctaact tatgggcaaa gtacttgcct tgccaggtgt atgggttttg cattcccatc |
| 5641 | acccacacac cctaaacaag ccaagtcagt gagcttcaag ttagagcctc cacctcaatg |
| 5701 | tgtacgtgga aagcaatcaa agatgatgcc tagcatccac ctctggccct catgtgcaga |
| 5761 | tgtacacaca ctgaattaca tacacgggac acacacatcc acacggaggc agtccatgac |
| 5821 | ttgcactggg gagatggtac cataggcgaa agtgccacag gcacagggcc aggctaattt |
| 5881 | agtcctgcag tcctgtgctc ttaagatgaa ggcacaaaga ggaaccccag gcgctccaac |
| 5941 | tagcatgcca ggcagtgaca agaccctgct tcaaatgaat cagagcccac attcagtatt |
| 6001 | gccctcttac ccgatgcgat gcccatgccc tcacatatga atgtgtatat atacatacat |
| 6061 | acgtaaaata attctttttt aaattataga catttttgtg tgaatgtttt gcctgaatgt |
| 6121 | gtgtgtgtgt gtgtgtgtgt gtgtgtgtgt gtgtgtgtgt gtatcaagta cattcctaga |
| 6181 | gcctacagag gtcaagggag ggcattggat ctggaactgg agtcacatga ggctgtgagc |
| 6241 | aactgtgtgg gttcctgggc ctttgcaaca gcagttagta ctcttcacca ctgagccatt |
| 6301 | tctccaatct caaaaagaag cattctttta aatgaagact gaaataaata agtaggactt |
| 6361 | gccttgg |

SEQ ID NO: 207 Mouse SMARCA4 Amino Acid Sequence isoform 2 (NP_035547.2)

| | |
|---|---|
| 1 | mstpdpplgg tprpgpspgp gpspgamlgp spgpspgsah smmgpspgpp saghpmptqg |
| 61 | pggypqdnmh qmhkpmesmh ekgmpddpry nqmkgmgmrs gahtgmappp spmdqhsqgy |
| 121 | psplggseha sspvpasgps sgpqmssgpg gapldgsdpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa plvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kiegerkrrq khqeylnsil |
| 481 | qhakdfreyh rsvtgklqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqpaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlkaie egtleeieee vrqkkssrkr |
| 1381 | krdseagsst pttstrsrdk deeskkqkkr grppaeklsp nppnltkkmk kivdavikyk |
| 1441 | dsssgrqlse vfiqlpsrke lpeyyelirk pvdfkkiker irnhkyrsln dlekdvmllc |
| 1501 | qnaqtfnleg sliyedsivl qsvftsvrqk iekeddsege eseeeeegee egsesesrsv |
| 1561 | kvkiklgrke kaqdrlkggr rrpsrgsrak pvvsdddsee eqeedrsgsg seed |

SEQ ID NO: 208 Mouse SMARCA4 cDNA Sequence variant 3 (NM_001174079.1;
CDS: 261-5102)

| | |
|---|---|
| 1 | ggcaagtgga gcgggtagac agggaggcgg gggcgcgcgg cgggcgcgtg cggtgggggg |
| 61 | gggtggcctg gcgaagccca gcgggcgcgc gcgcgaggct ttcccactcg cttggcagcg |
| 121 | gcggagacgg cttctttgtt tcctgaggag aagcgagacg cccactctgt ccccgacccc |
| 181 | tcgtggaggg ttgggggcgg cgccaggaag gttacggcgc cgttacctcc aggagaccag |
| 241 | tgcctgtagc tccagtaaag atgtctactc cagacccacc cttgggtggg actcctcggc |
| 301 | ctggtccttc cccaggccct ggtccttcac ctggtgcaat gctgggtcct agccctggcc |
| 361 | cctcaccagg ttctgcccac agcatgatgg ggccaagccc aggacctcct tcagcaggac |
| 421 | atcccatgcc cacccagggg cctggagggt acccccagga caacatgcat cagatgcaca |
| 481 | agcctatgga gtccatgcac gagaagggca tgcctgatga cccacgatac aaccagatga |
| 541 | aagggatggg catgcggtca ggggcccaca caggcatggc acctccacct agtcccatgg |
| 601 | accagcattc tcaaggttac ccctcacccc tcggcggctc tgaacatgcc tccagtcctg |
| 661 | tcccagccag tggcccatct tcaggccccc agatgtcctc tgggccagga ggggccccac |
| 721 | tagatggttc tgatccccag gccttgggac agcaaaacag aggcccaacc ccatttaacc |
| 781 | agaaccagct gcatcaactc agagctcaga taatggccta caagatgttg gccaggggcc |
| 841 | agccattgcc cgaccacctg cagatggccg tgcaaggcaa gcggccgatg cctggaatgc |
| 901 | agcaacagat gccaacacta cctccaccct cagtgtccgc cacaggaccc ggacctggac |
| 961 | ccggccctgg ccctggccct ggcccaggac cagcccctcc aaattacagt agaccccatg |
| 1021 | gtatgggagg gcccaacatg cctcccccag gaccctcagg tgtgcccccc gggatgcctg |
| 1081 | gtcagccgcc tggagggcct cccaagccat ggcctgaagg acccatggcc aatgctgctg |
| 1141 | cccccacaag caccccacag aagctgattc ctccgcaacc aacaggccgt ccttcacctg |
| 1201 | cacctcctgc tgtcccgcct gctgcctcac ctgtaatgcc accacaaaca cagtccccag |
| 1261 | ggcagccagc ccagcctgct ccattggtgc cactgcacca gaagcagagc cgaatcaccc |
| 1321 | ccatccagaa gccccgaggc cttgaccctg tggagatcct acaagagcgg gagtacaggc |
| 1381 | ttcaggctcg aatcgcacac agaattcagg aacttgaaaa cctccctggg tccctggctg |
| 1441 | gggaccttcg aaccaaagca accatcgaac tcaaggccct taggttgctg aacttccaga |
| 1501 | ggcagctgcg ccaggaggtg gtggtgtgca tgcgaagaga cacagccctg gagacagccc |
| 1561 | tcaatgccaa ggcctacaag cgcagcaaac gtcagtcact acgggaggcc cgcatcactg |
| 1621 | agaagttgga gaagcagcag aagattgaac aggagcgcaa gcgccgccag aagcaccagg |
| 1681 | agtacctcaa cagcattctg cagcatgcca aggacttcag ggagtatcac agatcagtca |
| 1741 | caggcaaact ccagaaactc accaaggctg tggccaccta ccatgccaac actgagcggg |
| 1801 | agcagaagaa agaaaatgag cgcattgaga aggagcgaat gcggaggctt atggctgaag |
| 1861 | atgaggaggg ctaccgcaaa ctcattgacc agaagaagga caagcgcctg gcctaccttc |
| 1921 | tgcagcagac agatgagtat gtggccaacc tcacagagct ggtgcggcag cacaaagctg |
| 1981 | cccaggttgc caaggagaag aagaagaaaa agaaaaagaa gaaggcagaa aatgctgaag |
| 2041 | gacagacacc tgctattgga ccagatggtg agcctctgga tgagaccagc cagatgagtg |
| 2101 | acctccctgt gaaggtgatc cacgtggaga gtggcaagat cctcactggc acagatgccc |
| 2161 | caaaagccgg gcagctggaa gcctggcttg aaatgaaccc agggtatgaa gtagccccca |
| 2221 | ggtcagacag tgaagaaagt ggctctgaag aggaggagga ggaggaggaa gaggagcagc |
| 2281 | ctcagcccgc acagccccct acactgcctg tggaagaaaa gaagaagatt ccagacccag |
| 2341 | acagcgatga tgtctctgag gtggacgccc gacacattat tgagaacgcc aagcaagatg |
| 2401 | tggacgatga gtacggtgtg tcccaggccc ttgctcgtgg cctgcagtct tactatgctg |
| 2461 | tggcccatgc agtcacagag agagtagata agcagtccgc cctcatggtc aacggtgtcc |
| 2521 | tcaaacagta ccagatcaag ggtttggagt ggctggtgtc cctgtacaac aacaacctga |
| 2581 | atggcatcct ggctgatgag atggggctgg ggaagaccat ccagaccatc gcgctcatca |
| 2641 | catacctcat ggagcacaag cgcatcaacg ggcctttcct catcatcgtg cctctctcga |
| 2701 | cactgtcaaa ctgggcgtat gaatttgaca agtgggcccc ctctgtggtg aaggtttctt |
| 2761 | acaagggctc tccagctgca aggcgagctt ttgtcccaca gcttcgcagt gggaagttca |
| 2821 | acgtcttact gaccacctat gaatatatca tcaaagacaa gcatatccta gccaagatcc |
| 2881 | gctggaagta catgattgtg gatgaaggcc accgcatgaa aaaccaccac tgcaagttga |
| 2941 | cgcaggtcct taacacacac tacgtggccc ctcggcgcct gcttcttaca ggcacaccac |
| 3001 | tgcagaacaa gctaccggag ctctgggccc tgcttaactt cctgctcccc actatcttca |
| 3061 | agagctgcag caccttcgaa cagtggttca atgcaccctt tgccatgact ggagaaaagg |
| 3121 | tggacctgaa tgaagaggag actatcctca ttattcgtcg cctacacaaa gttctgcggc |
| 3181 | ccttcctgct gcggcggctc aagaaggaag ttgaagccca gctccctgag aaggtagagt |
| 3241 | atgtcatcaa atgcgacatg tcagccctgc agcgtgtgct gtaccgtcac atgcaggcca |
| 3301 | aaggtgtgct gctgactgac ggctccgaga aggacaagaa gggcaaaggt ggcaccaaga |
| 3361 | cactgatgaa cactattatg caactgcgta agatctgcaa ccacccctac atgttccagc |
| 3421 | acatcgagga gtccttttct gagcacttgg ggttcaccgg cggcatcgtg caaggattgg |
| 3481 | acctttaccg tgcctcaggg aaatttgaac ttcttgatag aattctaccc aaactccgtg |
| 3541 | caacgaacca taaagtgctc ctcttttgcc aaatgacctc cctcatgacc atcatggaag |
| 3601 | actactttgc ataccgtggc ttcaaatacc tcaggcttga tggaaccaca aaagcagaag |
| 3661 | accggggcat gctgttgaaa acctttaatg aacctggctc tgagtatttc attttcctgc |
| 3721 | tcagtacccg tgctgggggg ctgggcctga atctgcagtc agctgacact gtgatcatct |
| 3781 | ttgacagtga ctggaatccc caccaggacc tgcaagcaca ggatcgagcc catcgcattg |
| 3841 | gacagcagaa tgaggtgcgt gttcttcgcc tgtgcacggt caacagtgtg gaagagaaga |
| 3901 | tactggctgc tgccaaatac aaactcaatg tggatcagaa ggtgatccag gcaggcatgt |
| 3961 | tcgaccagaa gtcgtccagc catgagaggc gtgccttcct gcaggccatc ctggagcacg |
| 4021 | aggagcagga tgaggaggaa gatgaggtgc ctgatgatga gaccgtcaac cagatgattg |
| 4081 | cccggcacga agaagagttt gacctcttca tgcgcatgga cttggaccgc cggcgtgaag |
| 4141 | aagcccgcaa ccccaagcgg aagccacgcc tgatggaaga ggatgagctc ccatcctgga |
| 4201 | tcatcaagga tgatgccgag gtggagcggc tgacatgtga agaggaagag gagaagatgt |
| 4261 | tcggccgtgg ttctcgccac cgcaaggagg tagactacag cgactcactg acagagaagc |
| 4321 | agtggctcaa ggctatcgag gagggcacgc tggaggagat cgaagaggag gtccggcaga |
| 4381 | agaaatcttc acgtaagcgt aagcgagaca gcgaggccgg ctcctccacc ccgaccacca |
| 4441 | gcacccgcag ccgtgacaag gatgaggaga gcaagaagca gaagaaacgt gggcggccac |
| 4501 | ctgctgagaa gctgtcccca aacccaccta acctcaccaa gaagatgaag aagatcgtgg |
| 4561 | atgctgtgat caagtacaaa gacagcagtg gacgtcagct cagcgaggtg ttcatccagc |
| 4621 | tcccctctcg caaggagctt cctgagtact atgagctcat ccgaaagcct gtggacttca |
| 4681 | agaagatcaa ggaacgcatc cgaaaccaca agtaccgcag cctcaatgac ctggagaagg |
| 4741 | atgtgatgct gctgtgccag aacgctcaga cgttcaacct cgagggttcc ctgatctatg |
| 4801 | aggactccat cgtcctgcag tctgtcttca ccagcgtacg gcagaagatt gagaaggagg |
| 4861 | acgacagtga aggcgaggaa agcgaggagg aggaggaggg cgaggaggaa ggctccgagt |
| 4921 | ctgagtcccg ctccgtcaag gtgaagatca agctgggccg caaggagaag gcccaggacc |
| 4981 | gactcaaggg gggccgccgg cggccaagcc ggggatcccg ggccaagccg gttgtgagtg |
| 5041 | acgatgacag tgaggaggag caggaggagg accgctcagg aagtggcagt gaggaagact |
| 5101 | gaaccagaca ttcctgagtc ctgaccccga ggcgctcgtc ccagccaaga tggagtagcc |
| 5161 | cttagcagtg atgggtagca ccagatgtag tttcgaactt ggagaactgt acacatgcaa |
| 5221 | tcttccacat ttttaggcag agaagtatag gcctgtctgt cggccctggc ctggcctcga |
| 5281 | gtctctacca gcattaactg tctagagagg ggacctcctg ggagcaccat ccacctcccc |
| 5341 | aggccccagt cactgtagct cagtggatgc atgcgcgtgc cggccgctcc ttgtactgta |
| 5401 | tcttactgga cagggccagc tctccaggag gctcacaggc ccagcgggta tgtcagtgtc |
| 5461 | actggagtca gacagtaata aattaaagca atgacaagcc accactggct ccctggactc |
| 5521 | cttgctgtca gcagtggctc cggggccaca gagaagaaag aaagactttt aggaactggg |
| 5581 | tctaacttat gggcaaagta cttgccttgc caggtgtatg ggttttgcat tcccatcacc |
| 5641 | cacacaccct aaacaagcca agtcagtgag cttcaagtta gagcctccac ctcaatgtgt |
| 5701 | acgtggaaag caatcaaaga tgatgcctag catccacctc tggccctcat gtgcagatgt |
| 5761 | acacacactg aattacatac acgggacaca cacatccaca cggaggcagt ccatgacttg |
| 5821 | cactggggag atggtaccat aggcgaaagt gccacaggca cagggccagg ctaatttagt |
| 5881 | cctgcagtcc tgtgctctta agatgaaggc acaaagagga accccaggcg ctccaactag |
| 5941 | catgccaggc agtgacaaga ccctgcttca aatgaatcag agcccacatt cagtattgcc |
| 6001 | ctcttacccg atgcgatgcc catgccctca catatgaatg tgtatatata catacatacg |
| 6061 | taaaataatt cttttttaaa ttatagacat ttttgtgtga atgttttgcc tgaatgtgtg |
| 6121 | tgtgtgtgtg tgtgtgtgtg tgtgtgtgtg tgtgtgtgta tcaagtacat tcctagagcc |
| 6181 | tacagaggtc aagggagggc attggatctg gaactggagt cacatgaggc tgtgagcaac |
| 6241 | tgtgtgggtt cctgggcctt tgcaacagca gttagtactc ttcaccactg agccatttct |
| 6301 | ccaatctcaa aaagaagcat tcttttaaat gaagactgaa ataaataagt aggacttgcc |
| 6361 | ttgg |

SEQ ID NO: 209 Mouse SMARCA4 Amino Acid Sequence isoform 3
(NP_001167550.1)

| | |
|---|---|
| 1 | mstpdpplgg tprpgpspgp gpspgamlgp spgpspgsah smmgpspgpp saghpmptqg |
| 61 | pggypqdnmh qmhkpmesmh ekgmpddpry nqmkgmgmrs gahtgmappp spmdqhsqgy |
| 121 | psplggseha sspvpasgps sgpqmssgpg gapldgsdpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa plvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfreyh rsvtgklqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqpaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdeee |
| 1261 | devpddetvn qmiarheeef dlfmrmdldr rreearnpkr kprlmeedel pswiikddae |
| 1321 | verltceeee ekmfgrgsrh rkevdysdsl tekqwlkaie egtleeieee vrqkkssrkr |
| 1381 | krdseagsst pttstrsrdk deeskkqkkr grppaeklsp nppnltkkmk kivdavikyk |
| 1441 | dssgrqlsev fiqlpsrkel peyyelirkp vdfkkikeri rnhkyrsind lekdvmllcq |
| 1501 | naqtfnlegs liyedsivlq svftsvrqki ekeddsegee seeeeegeee gsesesrsvk |
| 1561 | vkiklgrkek aqdrlkggrr rpsrgsrakp vvsdddseee qeedrsgsgs eed |

SEQ ID NO: 210 Mouse SMARCA4 cDNA Sequence variant 4 (NM_001357764.1;
CDS: 261-5204)

| | |
|---|---|
| 1 | ggcaagtgga gcgggtagac agggaggcgg gggcgcgcgg cgggcgcgtg cggtgggggg |
| 61 | gggtggcctg gcgaagccca gcgggcgcgc gcgcgaggct ttcccactcg cttggcagcg |
| 121 | gcggagacgg cttctttgtt tcctgaggag aagcgagacg cccactctgt ccccgacccc |
| 181 | tcgtggaggg ttgggggcgg cgccaggaag gttacggcgc cgttacctcc aggagaccag |
| 241 | tgcctgtagc tccagtaaag atgtctactc cagacccacc cttgggtggg actcctcggc |
| 301 | ctggtccttc cccaggccct ggtccttcac ctggtgcaat gctgggtcct agccctggcc |
| 361 | cctcaccagg ttctgcccac agcatgatgg ggccaagccc aggacctcct tcagcaggac |
| 421 | atcccatgcc cacccagggg cctggagggt acccccagga caacatgcat cagatgcaca |
| 481 | agcctatgga gtccatgcac gagaagggca tgcctgatga cccacgatac aaccagatga |
| 541 | aagggatggg catgcggtca ggggcccaca caggcatggc acctccacct agtcccatgg |
| 601 | accagcattc tcaaggttac ccctcacccc tcggCggctc tgaacatgcc tccagtcctg |
| 661 | tcccagccag tggcccatct tcaggccccc agatgtcctc tgggccagga ggggccccac |
| 721 | tagatggttc tgatccccag gccttgggac agcaaaacag aggcccaacc ccatttaacc |
| 781 | agaaccagct gcatcaactc agagctcaga taatggccta caagatgttg gccaggggcc |
| 841 | agccattgcc cgaccacctg cagatggccg tgcaaggcaa gcggccgatg cctggaatgc |
| 901 | agcaacagat gccaacacta cctccaccct cagtgtccgc cacaggaccc ggacctggac |
| 961 | ccggccctgg ccctggccct ggcccaggac cagcccctcc aaattacagt agaccccatg |
| 1021 | gtatgggagg gcccaacatg cctcccccag gaccctcagg tgtgcccccc gggatgcctg |
| 1081 | gtcagccgcc tggagggcct cccaagccat ggcctgaagg acccatggcc aatgctgctg |
| 1141 | cccccacaag caccccacag aagctgattc ctccgcaacc aacaggccgt ccttcacctg |
| 1201 | cacctcctgc tgtcccgcct gctgcctcac ctgtaatgcc accacaaaca cagtccccag |
| 1261 | ggcagccagc ccagcctgct ccattggtgc cactgcacca gaagcagagc cgaatcaccc |
| 1321 | ccatccagaa gccccgaggc cttgaccctg tggagatcct acaagagcgg gagtacaggc |
| 1381 | ttcaggctcg aatcgcacac agaattcagg aacttgaaaa cctccctggg tccctggctg |
| 1441 | gggaccttcg aaccaaagca accatcgaac tcaaggccct taggttgctg aacttccaga |
| 1501 | ggcagctgcg ccaggaggtg gtggtgtgca tgcgaagaga cacagccctg gagacagccc |
| 1561 | tcaatgccaa ggcctacaag cgcagcaaac gtcagtcact acgggaggcc cgcatcactg |
| 1621 | agaagttgga gaagcagcag aagattgaac aggagcgcaa gcgccgccag aagcaccagg |
| 1681 | agtacctcaa cagcattctg cagcatgcca aggacttcag ggagtatcac agatcagtca |
| 1741 | caggcaaact ccagaaactc accaaggctg tggccaccta ccatgccaac actgagcggg |
| 1801 | agcagaagaa agaaaatgag cgcattgaga aggagcgaat gcggaggctt atggctgaag |
| 1861 | atgaggaggg ctaccgcaaa ctcattgacc agaagaagga caagcgcctg gcctaccttc |
| 1921 | tgcagcagac agatgagtat gtggccaacc tcacagagct ggtgcggcag cacaaagctg |
| 1981 | cccaggttgc caaggagaag aagaagaaaa agaaaaagaa gaaggcagaa aatgctgaag |
| 2041 | gacagacacc tgctattgga ccagatggtg agcctctgga tgagaccagc cagatgagtg |
| 2101 | acctccctgt gaaggtgatc cacgtggaga gtggcaagat cctcactggc acagatgccc |
| 2161 | caaaagccgg gcagctggaa gcctggcttg aaatgaaccc agggtatgaa gtagccccca |
| 2221 | ggtcagacag tgaagaaagt ggctctgaag aggaggagga ggaggaggaa gaggagcagc |
| 2281 | ctcagcccgc acagccccct acactgcctg tggaagaaaa gaagaagatt ccagacccag |
| 2341 | acagcgatga tgtctctgag gtggacgccc gacacattat tgagaacgcc aagcaagatg |
| 2401 | tggacgatga gtacggtgtg tcccaggccc ttgctcgtgg cctgcagtct tactatgctg |
| 2461 | tggcccatgc agtcacagag agagtagata agcagtccgc cctcatggtc aacggtgtcc |
| 2521 | tcaaacagta ccagatcaag ggtttggagt ggctggtgtc cctgtacaac aacaacctga |
| 2581 | atggcatcct ggctgatgag atggggctgg ggaagaccat ccagaccatc gcgctcatca |
| 2641 | catacctcat ggagcacaag cgcatcaacg ggcctttcct catcatcgtg cctctctcga |
| 2701 | cactgtcaaa ctgggcgtat gaatttgaca agtgggcccc ctctgtggtg aaggtttctt |
| 2761 | acaagggctc tccagctgca aggcgagctt ttgtcccaca gcttcgcagt gggaagttca |
| 2821 | acgtcttact gaccacctat gaatatatca tcaaagacaa gcatatccta gccaagatcc |
| 2881 | gctggaagta catgattgtg gatgaaggcc accgcatgaa aaaccaccac tgcaagttga |
| 2941 | cgcaggtcct taacacacac tacgtggccc ctcggcgcct gcttcttaca ggcacaccac |
| 3001 | tgcagaacaa gctaccggag ctctgggccc tgcttaactt cctgctcccc actatcttca |
| 3061 | agagctgcag caccttcgaa cagtggttca atgcaccctt tgccatgact ggagaaaagg |
| 3121 | tggacctgaa tgaagaggag actatcctca ttattcgtcg cctacacaaa gttctgcggc |
| 3181 | ccttcctgct gcggcggctc aagaaggaag ttgaagccca gctccctgag aaggtagagt |
| 3241 | atgtcatcaa atgcgacatg tcagccctgc agcgtgtgct gtaccgtcac atgcaggcca |
| 3301 | aaggtgtgct gctgactgac ggctccgaga aggacaagaa gggcaaaggt ggcaccaaga |
| 3361 | cactgatgaa cactattatg caactgcgta agatctgcaa ccacccctac atgttccagc |
| 3421 | acatcgagga gtccttttct gagcacttgg ggttcaccgg cggcatcgtg caaggattgg |
| 3481 | acctttaccg tgcctcaggg aaatttgaac ttcttgatag aattctaccc aaactccgtg |
| 3541 | caacgaacca taaagtgctc ctcttttgcc aaatgacctc cctcatgacc atcatggaag |
| 3601 | actactttgc ataccgtggc ttcaaatacc tcaggcttga tggaaccaca aaagcagaag |
| 3661 | accggggcat gctgttgaaa acctttaatg aacctggctc tgagtatttc attttcctgc |
| 3721 | tcagtacccg tgctgggggg ctgggcctga atctgcagtc agctgacact gtgatcatct |
| 3781 | ttgacagtga ctggaatccc caccaggacc tgcaagcaca ggatcgagcc catcgcattg |
| 3841 | gacagcagaa tgaggtgcgt gttcttcgcc tgtgcacggt caacagtgtg gaagagaaga |
| 3901 | tactggctgc tgccaaatac aaactcaatg tggatcagaa ggtgatccag gcaggcatgt |
| 3961 | tcgaccagaa gtcgtccagc catgagaggc gtgccttcct gcaggccatc ctggagcacg |
| 4021 | aggagcagga tgagagcaga cactgcagca cgggcagcgg cagtgccagc ttcgcccaca |
| 4081 | ctgcccctcc gccagcgggc gtcaaccccg acttggagga gccacctcta aaggaggaag |
| 4141 | atgaggtgcc tgatgatgag accgtcaacc agatgattgc ccggcacgaa gaagagtttg |
| 4201 | acctcttcat gcgcatggac ttggaccgcc ggcgtgaaga agcccgcaac cccaagcgga |
| 4261 | agccacgcct gatggaagag gatgagctcc catcctggat catcaaggat gatgccgagg |
| 4321 | tggagcggct gacatgtgaa gaggaagagg agaagatgtt cggccgtggt tctcgccacc |
| 4381 | gcaaggaggt agactacagc gactcactga cagagaagca gtggctcaag gctatcgagg |
| 4441 | agggcacgct ggaggagatc gaagaggagg tccggcagaa gaaatcttca cgtaagcgta |
| 4501 | agcgagacag cgaggccggc tcctccaccc cgaccaccag cacccgcagc cgtgacaagg |
| 4561 | atgaggagag caagaagcag aagaaacgtg ggcggccacc tgctgagaag ctgtccccaa |
| 4621 | acccacctaa cctcaccaag aagatgaaga agatcgtgga tgctgtgatc aagtacaaag |
| 4681 | acagcagcag tggacgtcag ctcagcgagg tgttcatcca gctcccctct cgcaaggagc |
| 4741 | ttcctgagta ctatgagctc atccgaaagc ctgtggactt caagaagatc aaggaacgca |
| 4801 | tccgaaacca caagtaccgc agcctcaatg acctggagaa ggatgtgatg ctgctgtgcc |
| 4861 | agaacgctca gacgttcaac ctcgagggtt ccctgatcta tgaggactcc atcgtcctgc |
| 4921 | agtctgtctt caccagcgta cggcagaaga ttgagaagga ggacgacagt gaaggcgagg |
| 4981 | aaagcgagga ggaggaggag ggcgaggagg aaggctccga gtctgagtcc cgctccgtca |
| 5041 | aggtgaagat caagctgggc cgcaaggaga aggcccagga ccgactcaag gggggccgcc |
| 5101 | ggcggccaag ccggggatcc cgggccaagc cggttgtgag tgacgatgac agtgaggagg |
| 5161 | agcaggagga ggaccgctca ggaagtggca gtgaggaaga ctgaaccaga cattcctgag |
| 5221 | tcctgacccc gaggcgctcg tcccagccaa gatggagtag cccttagcag tgatgggtag |
| 5281 | caccagatgt agtttcgaac ttggagaact gtacacatgc aatcttccac atttttaggc |
| 5341 | agagaagtat aggcctgtct gtcggccctg gcctggcctc gagtctctac cagcattaac |
| 5401 | tgtctagaga ggggacctcc tgggagcacc atccacctcc ccaggcccca gtcactgtag |
| 5461 | ctcagtggat gcatgcgcgt gccggccgct ccttgtactg tatcttactg gacagggcca |
| 5521 | gctctccagg aggctcacag gcccagcggg tatgtcagtg tcactggagt cagacagtaa |
| 5581 | taaattaaag caatgacaag ccaccactgg ctccctggac tccttgctgt cagcagtggc |
| 5641 | tccggggcca cagagaagaa agaaagactt ttaggaactg ggtctaactt atgggcaaag |
| 5701 | tacttgcctt gccaggtgta tgggttttgc attcccatca cccacacacc ctaaacaagc |
| 5761 | caagtcagtg agcttcaagt tagagcctcc acctcaatgt gtacgtggaa agcaatcaaa |
| 5821 | gatgatgcct agcatccacc tctggccctc atgtgcagat gtacacacac tgaattacat |
| 5881 | acacgggaca cacacatcca cacggaggca gtccatgact tgcactgggg agatggtacc |
| 5941 | ataggcgaaa gtgccacagg cacagggcca ggctaattta gtcctgcagt cctgtgctct |
| 6001 | taagatgaag gcacaaagag gaaccccagg cgctccaact agcatgccag gcagtgacaa |
| 6061 | gaccctgctt caaatgaatc agagcccaca ttcagtattg ccctcttacc cgatgcgatg |
| 6121 | cccatgccct cacatatgaa tgtgtatata tacatacata cgtaaaataa ttctttttta |
| 6181 | aattatagac atttttgtgt gaatgttttg cctgaatgtg tgtgtgtgtg tgtgtgtgtg |
| 6241 | tgtgtgtgtg tgtgtgtgtg tatcaagtac attcctagag cctacagagg tcaagggagg |
| 6301 | gcattggatc tggaactgga gtcacatgag gctgtgagca actgtgtggg ttcctgggcc |
| 6361 | tttgcaacag cagttagtac tcttcaccac tgagccattt ctccaatctc aaaaagaagc |
| 6421 | attcttttaa atgaagactg aaataaataa gtaggacttg ccttgg |

SEQ ID NO: 211 Mouse SMARCA4 Amino Acid Sequence isoform 4
(NP_001344693.1)

| | |
|---|---|
| 1 | mstpdpplgg tprpgpspgp gpspgamlgp spgpspgsah smmgpspgpp saghpmptqg |
| 61 | pggypqdnmh qmhkpmesmh ekgmpddpry nqmkgmgmrs gahtgmappp spmdqhsqgy |
| 121 | psplggseha sspvpasgps sgpqmssgpg gapldgsdpq algqqnrgpt pfnqnqlhql |
| 181 | raqimaykml argqplpdhl qmavqgkrpm pgmqqqmptl pppsvsatgp gpgpgpgpgp |
| 241 | gpgpappnys rphgmggpnm pppgpsgvpp gmpgqppggp pkpwpegpma naaaptstpq |
| 301 | klippqptgr pspappavpp aaspvmppqt qspgqpaqpa plvplhqkqs ritpiqkprg |
| 361 | ldpveilqer eyrlqariah riqelenlpg slagdlrtka tielkalrll nfqrqlrqev |
| 421 | vvcmrrdtal etalnakayk rskrqslrea riteklekqq kieqerkrrq khqeylnsil |
| 481 | qhakdfreyh rsvtgklqkl tkavatyhan tereqkkene riekermrrl maedeegyrk |
| 541 | lidqkkdkrl ayllqqtdey vanltelvrq hkaaqvakek kkkkkkkkae naegqtpaig |
| 601 | pdgepldets qmsdlpvkvi hvesgkiltg tdapkagqle awlemnpgye vaprsdsees |
| 661 | gseeeeeeee eeqpqpaqpp tlpveekkki pdpdsddvse vdarhiiena kqdvddeygv |
| 721 | sqalarglqs yyavahavte rvdkqsalmv ngvlkqyqik glewlvslyn nnlngilade |
| 781 | mglgktiqti alitylmehk ringpfliiv plstlsnway efdkwapsvv kvsykgspaa |
| 841 | rrafvpqlrs gkfnvlltty eyiikdkhil akirwkymiv deghrmknhh ckltqvlnth |
| 901 | yvaprrlllt gtplqnklpe lwallnfllp tifkscstfe qwfnapfamt gekvdlneee |
| 961 | tiliirrlhk vlrpfllrrl kkeveaqlpe kveyvikcdm salqrvlyrh mqakgvlltd |
| 1021 | gsekdkkgkg gtktlmntim qlrkicnhpy mfqhieesfs ehlgftggiv qgldlyrasg |
| 1081 | kfelldrilp klratnhkvl lfcqmtslmt imedyfayrg fkylrldgtt kaedrgmllk |
| 1141 | tfnepgseyf ifllstragg lglnlqsadt viifdsdwnp hqdlqaqdra hrigqqnevr |
| 1201 | vlrlctvnsv eekilaaaky klnvdqkviq agmfdqksss herraflqai leheeqdesr |
| 1261 | hcstgsgsas fahtapppag vnpdleeppl keedevpdde tvnqmiarhe eefdlfmrmd |
| 1321 | ldrrreearn pkrkprlmee delpswiikd daeverltce eeeekmfgrg srhrkevdys |
| 1381 | dsltekqwlk aieegtleei eeevrqkkss rkrkrdseag sstpttstrs rdkdeeskkq |
| 1441 | kkrgrppaek lspnppnltk kmkkivdavi kykdsssgrq lsevfiqlps rkelpeyyel |
| 1501 | irkpvdfkki kerirnhkyr slndlekdvm llcqnaqtfn legsliyeds ivlqsvftsv |
| 1561 | rqkiekedds egeeseeeee geeegseses rsvkvkiklg rkekagdrlk ggrrrpsrgs |
| 1621 | rakpvvsddd seeeqeedrs gsgseed |

SEQ ID NO: 212 Mouse SMARCA4 cDNA Sequence variant 1 (NM_001174078.1;
261-5114)

| | |
|---|---|
| 1 | ggcaagtgga gcgggtagac agggaggcgg gggcgcgcgg cgggcgcgtg cggtgggggg |
| 61 | gggtggcctg gcgaagccca gcgggcgcgc gcgcgaggct ttcccactcg cttggcagcg |
| 121 | gcggagacgg cttctttgtt tcctgaggag aagcgagacg cccactctgt ccccgacccc |
| 181 | tcgtggaggg ttgggggcgg cgccaggaag gttacggcgc cgttacctcc aggagaccag |
| 241 | tgcctgtagc tccagtaaag atgtctactc cagacccacc cttgggtggg actcctcggc |
| 301 | ctggtccttc cccaggccct ggtccttcac ctggtgcaat gctgggtcct agccctggcc |
| 361 | cctcaccagg ttctgcccac agcatgatgg ggccaagccc aggacctcct tcagcaggac |
| 421 | atcccatgcc cacccagggg cctggagggt acccccagga caacatgcat cagatgcaca |
| 481 | agcctatgga gtccatgcac gagaagggca tgcctgatga cccacgatac aaccagatga |
| 541 | aagggatggg catgcggtca ggggcccaca caggcatggc acctccacct agtcccatgg |
| 601 | accagcattc tcaaggttac ccctcacccc tcggcggctc tgaacatgcc tccagtcctg |
| 661 | tcccagccag tggcccatct tcaggccccc agatgtcctc tgggccagga ggggccccac |
| 721 | tagatggttc tgatccccag gccttgggac agcaaaacag aggcccaacc ccatttaacc |
| 781 | agaaccagct gcatcaactc agagctcaga taatggccta caagatgttg gccaggggcc |
| 841 | agccattgcc cgaccacctg cagatggccg tgcaaggcaa gcggccgatg cctggaatgc |
| 901 | agcaacagat gccaacacta cctccaccct cagtgtccgc cacaggaccc ggacctggac |
| 961 | ccggccctgg ccctggccct ggcccaggac cagcccctcc aaattacagt agaccccatg |
| 1021 | gtatgggagg gcccaacatg cctcccccag gaccctcagg tgtgcccccc gggatgcctg |
| 1081 | gtcagccgcc tggagggcct cccaagccat ggcctgaagg acccatggcc aatgctgctg |
| 1141 | cccccacaag caccccacag aagctgattc ctccgcaacc aacaggccgt ccttcacctg |
| 1201 | cacctcctgc tgtcccgcct gctgcctcac ctgtaatgcc accacaaaca cagtccccag |
| 1261 | ggcagccagc ccagcctgct ccattggtgc cactgcacca gaagcagagc cgaatcaccc |
| 1321 | ccatccagaa gccccgaggc cttgaccctg tggagatcct acaagagcgg gagtacaggc |
| 1381 | ttcaggctcg aatcgcacac agaattcagg aacttgaaaa cctccctggg tccctggctg |
| 1441 | gggaccttcg aaccaaagca accatcgaac tcaaggccct taggttgctg aacttccaga |
| 1501 | ggcagctgcg ccaggaggtg gtggtgtgca tgcgaagaga cacagccctg gagacagccc |
| 1561 | tcaatgccaa ggcctacaag cgcagcaaac gtcagtcact acgggaggcc cgcatcactg |
| 1621 | agaagttgga gaagcagcag aagattgaac aggagcgcaa gcgccgccag aagcaccagg |
| 1681 | agtacctcaa cagcattctg cagcatgcca aggacttcag ggagtatcac agatcagtca |
| 1741 | caggcaaact ccagaaactc accaaggctg tggccaccta ccatgccaac actgagcggg |
| 1801 | agcagaagaa agaaaatgag cgcattgaga aggagcgaat gcggaggctt atggctgaag |
| 1861 | atgaggaggg ctaccgcaaa ctcattgacc agaagaagga caagcgcctg gcctaccttc |
| 1921 | tgcagcagac agatgagtat gtggccaacc tcacagagct ggtgcggcag cacaaagctg |
| 1981 | cccaggttgc caaggagaag aagaagaaaa agaaaaagaa gaaggcagaa aatgctgaag |
| 2041 | gacagacacc tgctattgga ccagatggtg agcctctgga tgagaccagc cagatgagtg |
| 2101 | acctccctgt gaaggtgatc cacgtggaga gtggcaagat cctcactggc acagatgccc |
| 2161 | caaaagccgg gcagctggaa gcctggcttg aaatgaaccc agggtatgaa gtagccccca |
| 2221 | ggtcagacag tgaagaaagt ggctctgaag aggaggagga ggaggaggaa gaggagcagc |
| 2281 | ctcagcccgc acagccccct acactgcctg tggaagaaaa gaagaagatt ccagacccag |
| 2341 | acagcgatga tgtctctgag gtggacgccc gacacattat tgagaacgcc aagcaagatg |
| 2401 | tggacgatga gtacggtgtg tcccaggccc ttgctcgtgg cctgcagtct tactatgctg |
| 2461 | tggcccatgc agtcacagag agagtagata agcagtccgc cctcatggtc aacggtgtcc |
| 2521 | tcaaacagta ccagatcaag ggtttggagt ggctggtgtc cctgtacaac aacaacctga |
| 2581 | atggcatcct ggctgatgag atggggctgg ggaagaccat ccagaccatc gcgctcatca |
| 2641 | catacctcat ggagcacaag cgcatcaacg ggcctttcct catcatcgtg cctctctcga |
| 2701 | cactgtcaaa ctgggcgtat gaatttgaca agtgggcccc ctctgtggtg aaggtttctt |
| 2761 | acaagggctc tccagctgca aggcgagctt ttgtcccaca gcttcgcagt gggaagttca |
| 2821 | acgtcttact gaccacctat gaatatatca tcaaagacaa gcatatccta gccaagatcc |
| 2881 | gctggaagta catgattgtg gatgaaggcc accgcatgaa aaaccaccac tgcaagttga |
| 2941 | cgcaggtcct taacacacac tacgtggccc ctcggcgcct gcttcttaca ggcacaccac |
| 3001 | tgcagaacaa gctaccggag ctctgggccc tgcttaactt cctgctcccc actatcttca |
| 3061 | agagctgcag caccttcgaa cagtggttca atgcaccctt tgccatgact ggagaaaagg |
| 3121 | tggacctgaa tgaagaggag actatcctca ttattcgtcg cctacacaaa gttctgcggc |
| 3181 | ccttcctgct gcggcggctc aagaaggaag ttgaagccca gctccctgag aaggtagagt |
| 3241 | atgtcatcaa atgcgacatg tcagccctgc agcgtgtgct gtaccgtcac atgcaggcca |
| 3301 | aaggtgtgct gctgactgac ggctccgaga aggacaagaa gggcaaaggt ggcaccaaga |
| 3361 | cactgatgaa cactattatg caactgcgta agatctgcaa ccacccctac atgttccagc |
| 3421 | acatcgagga gtccttttct gagcacttgg ggttcaccgg cggcatcgtg caaggattgg |
| 3481 | acctttaccg tgcctcaggg aaatttgaac ttcttgatag aattctaccc aaactccgtg |
| 3541 | caacgaacca taaagtgctc ctcttttgcc aaatgacctc cctcatgacc atcatggaag |
| 3601 | actactttgc ataccgtggc ttcaaatacc tcaggcttga tggaaccaca aaagcagaag |
| 3661 | accggggcat gctgttgaaa acctttaatg aacctggctc tgagtatttc attttcctgc |
| 3721 | tcagtacccg tgctgggggg ctgggcctga atctgcagtc agctgacact gtgatcatct |
| 3781 | ttgacagtga ctggaatccc caccaggacc tgcaagcaca ggatcgagcc catcgcattg |
| 3841 | gacagcagaa tgaggtgcgt gttcttcgcc tgtgcacggt caacagtgtg gaagagaaga |
| 3901 | tactggctgc tgccaaatac aaactcaatg tggatcagaa ggtgatccag gcaggcatgt |
| 3961 | tcgaccagaa gtcgtccagc catgagaggc gtgccttcct gcaggccatc ctggagcacg |
| 4021 | aggagcagga tgaggaggaa gatgaggtgc ctgatgatga gaccgtcaac cagatgattg |
| 4081 | cccggcacga agaagagttt gacctcttca tgcgcatgga cttggaccgc cggcgtgaag |
| 4141 | aagcccgcaa ccccaagcgg aagccacgcc tgatggaaga ggatgagctc ccatcctgga |
| 4201 | tcatcaagga tgatgccgag gtggagcggc tgacatgtga agaggaagag gagaagatgt |
| 4261 | tcggccgtgg ttctcgccac cgcaaggagg tagactacag cgactcactg acagagaagc |
| 4321 | agtggctcaa gaccctgaag gctatcgagg agggcacgct ggaggagatc gaagaggagg |
| 4381 | tccggcagaa gaaatcttca cgtaagcgta agcgagacag cgaggccggc tcctccaccc |
| 4441 | cgaccaccag cacccgcagc cgtgacaagg atgaggagag caagaagcag aagaaacgtg |
| 4501 | ggcggccacc tgctgagaag ctgtccccaa acccacctaa cctcaccaag aagatgaaga |
| 4561 | agatcgtgga tgctgtgatc aagtacaaag acagcagcag tggacgtcag ctcagcgagg |
| 4621 | tgttcatcca gctcccctct cgcaaggagc ttcctgagta ctatgagctc atccgaaagc |
| 4681 | ctgtggactt caagaagatc aaggaacgca tccgaaacca caagtaccgc agcctcaatg |
| 4741 | acctggagaa ggatgtgatg ctgctgtgcc agaacgctca gacgttcaac ctcgagggtt |
| 4801 | ccctgatcta tgaggactcc atcgtcctgc agtctgtctt caccagcgta cggcagaaga |
| 4861 | ttgagaagga ggacgacagt gaaggcgagg aaagcgagga ggaggaggag ggcgaggagg |
| 4921 | aaggctccga gtctgagtcc cgctccgtca aggtgaagat caagctgggc cgcaaggaga |
| 4981 | aggcccagga ccgactcaag gggggccgcc ggcggccaag ccggggatcc cgggccaagc |
| 5041 | cggttgtgag tgacgatgac agtgaggagg agcaggagga ggaccgctca ggaagtggca |
| 5101 | gtgaggaaga ctgaaccaga cattcctgag tcctgacccc gaggcgctcg tcccagccaa |
| 5161 | gatggagtag cccttagcag tgatgggtag caccagatgt agtttcgaac ttggagaact |
| 5221 | gtacacatgc aatcttccac atttttaggc agagaagtat aggcctgtct gtcggccctg |
| 5281 | gcctggcctc gagtctctac cagcattaac tgtctagaga ggggacctcc tgggagcacc |
| 5341 | atccacctcc ccaggcccca gtcactgtag ctcagtggat gcatgcgcgt gccggccgct |
| 5401 | ccttgtactg tatcttactg gacagggcca gctctccagg aggctcacag gcccagcggg |
| 5461 | tatgtcagtg tcactggagt cagacagtaa taaattaaag caatgacaag ccaccactgg |
| 5521 | ctccctggac tccttgctgt cagcagtggc tccggggcca cagagaagaa agaaagactt |
| 5581 | ttaggaactg ggtctaactt atgggcaaag tacttgcctt gccaggtgta tgggttttgc |
| 5641 | attcccatca cccacacacc ctaaacaagc caagtcagtg agcttcaagt tagagcctcc |
| 5701 | acctcaatgt gtacgtggaa agcaatcaaa gatgatgcct agcatccacc tctggccctc |
| 5761 | atgtgcagat gtacacacac tgaattacat acacgggaca cacacatcca cacggaggca |
| 5821 | gtccatgact tgcactgggg agatggtacc ataggcgaaa gtgccacagg cacagggcca |
| 5881 | ggctaattta gtcctgcagt cctgtgctct taagatgaag gcacaaagag gaaccccagg |
| 5941 | cgctccaact agcatgccag gcagtgacaa gaccctgctt caaatgaatc agagcccaca |
| 6001 | ttcagtattg ccctcttacc cgatgcgatg cccatgccct cacatatgaa tgtgtatata |
| 6061 | tacatacata cgtaaaataa ttctttttta aattatagac atttttgtgt gaatgttttg |
| 6121 | cctgaatgtg tgtgtgtgtg tgtgtgtgtg tgtgtgtgtg tgtgtgtgtg tatcaagtac |
| 6181 | attcctagag cctacagagg tcaagggagg gcattggatc tggaactgga gtcacatgag |
| 6241 | gctgtgagca actgtgtggg ttcctgggcc tttgcaacag cagttagtac tcttcaccac |
| 6301 | tgagccattt ctccaatctc aaaaagaagc attcttttaa atgaagactg aaataaataa |
| 6361 | gtaggacttg ccttgg |

SEQ ID NO: 213 HumanHistone H3.1 Amino Acid Sequence (NP_003520.1)

| | |
|---|---|
| 1 | martkqtark stggkaprkq latkaarksa patggvkkph ryrpgtvalr eirryqkste |
| 61 | llirklpfqr lvreiaqdfk tdlrfqssav malqeaceay lvglfedtnl caihakrvti |
| 121 | mpkdiqlarr irgera |

SEQ ID NO: 214 Mouse Histone H3.1 Amino Acid Sequence (NP_038578.2):

| | |
|---|---|
| 1 | martkqtark stggkaprkq latkaarksa patggvkkph ryrpgtvalr eirryqkste |
| 61 | llirklpfqr lvreiaqdfk tdlrfqssav malqeaceay lvglfedtnl caihakrvti |
| 121 | mpkdiqlarr irgera |

SEQ ID NO: 215 Human Histone H3.2 Amino Acid Sequence (NP_001005464.1):

| | |
|---|---|
| 1 | martkqtark stggkaprkq latkaarksa patggvkkph ryrpgtvalr eirryqkste |
| 61 | llirklpfqr lvreiaqdfk tdlrfqssav malqeaseay lvglfedtnl caihakrvti |
| 121 | mpkdiqlarr irgera |

SEQ ID NO: 216 Mouse Histone H3.2 Amino Acid Sequence (NP_835587.1):

| | |
|---|---|
| 1 | martkqtark stggkaprkq latkaarksa patggvkkph ryrpgtvalr eirryqkste |
| 61 | llirklpfqr lvreiaqdfk tdlrfqssav malqeaseay lvglfedtnl caihakrvti |
| 121 | mpkdiqlarr irgera |

SEQ ID NO: 217 Human Histone H3.3 Amino Acid Sequence (NP_002098.1):

| | |
|---|---|
| 1 | martkqtark stggkaprkq latkaarksa pstggvkkph ryrpgtvalr eirryqkste |
| 61 | llirklpfqr lvreiaqdfk tdlrfqsaai galqeaseay lvglfedtnl caihakrvti |
| 121 | mpkdiqlarr irgera |

SEQ ID NO: 218 Mouse Histone H3.3 Amino Acid Sequence (NP_032237.1);

| | |
|---|---|
| 1 | martkqtark stggkaprkq latkaarksa pstggvkkph ryrpgtvalr eirryqkste |
| 61 | llirklpfqr lvreiaqdfk tdlrfqsaai galqeaseay lvglfedtnl caihakrvti |
| 121 | mpkdiqlarr irgera |

H2A protein and cDNA sequences described herein, including human, mouse, rat, and Xenopus H2A sequences
H2B protein and cDNA sequences described herein, including human, mouse, rat, and Xenopus H2B sequences

TABLE 2
SS18-SSX fusion protein sequences
MSVAFAAPRQRGKGEITPAAIQKMLDDNNHLIQCIMDSQNKGKTSECSQYQQMLHTNLVYLATIADSNQN
MQSLLPAPPTQNMPMGPGGMNQSGPPPPPPSHNMPSDGMVGGGPPAPHMQNQMNGQMPGPNHMPMQGPGP
NQLNMTNSSMNMPSSSHGSMGGYNHSVPSSQSMPVQNQMTMSQGQPMGNYGPRPNMSMQPNQGPMMHQQP
PSQQYNMPQGGGQHYQGQQPPMGMMGQVNQGNHMMGQPQIPPYRPPQQGPPQQYSGQEDYYGDQYSHGGQ
GPPEGMNQQYYPDGNSQYGQQQDAYQGPPPQQGYPPQQQQYPGQQGYPGQQQGYGPSQGGPGPQYPNYPQ
GQGQQYGGYRPTQPGPPQPPQQRPYGYDQIMPKKPAEDENDSKGVSEASGPQNDGKQLH
PPGKANISEKINKRSGPKPGKHAWTHRLRERKQLVIYEETSDPEEDDE (SEQ ID NO: 225)
SS18-SSX fusion protein cDNA sequences
SS18 AA 1-379 aa + SSX1 (C-terminal 78 AA)
ATGTCTGTGGCTTTCGCGGCCCCGAGGCAGCGAGGCAAGGGGGAGATCACTCCCGCTGCGATTCAGAAGA
TGTTGGATGACAATAACCATCTTATTCAGTGTATAATGGACTCTCAGAATAAAGGAAAGACCTCAGAGTG
TTCTCAGTATCAGCAGATGTTGCACACAAACTTGGTATACCTTGCTACAATAGCAGATTCTAATCAAAAT
ATGCAGTCTCTTTTACCAGCACCACCCACACAGAATATGCCTATGGGTCCTGGAGGGATGAATCAGAGCG
GCCCTCCCCCACCTCCACGCTCTCACAACATGCCTTCAGATGGAATGGTAGGTGGGGGTCCTCCTGCACC
GCACATGCAGAACCAGATGAACGGCCAGATGCCTGGGCCTAACCATATGCCTATGCAGGGACCTGGACCC
AATCAACTCAATATGACAAACAGTTCCATGAATATGCCTTCAAGTAGCCATGGATCCATGGGAGGTTACA
ACCATTCTGTGCCATCATCACAGAGCATGCCAGTACAGAATCAGATGACAATGAGTCAGGGACAACCAAT
GGGAAACTATGGTCCCAGACCAAATATGAGTATGCAGCCAAACCAAGGTCCAATGATGCATCAGCAGCCT
CCTTCTCAGCAATACAATATGCCACAGGGAGGCGGACAGCATTACCAAGGACAGCAGCCACCTATGGGAA
TGATGGGTCAAGTTAACCAAGGCAATCATATGATGGGTCAGAGACAGATTCCTCCCTATAGACCTCCTCA
ACAGGGCCCACCACAGCAGTACTCAGGCCAGGAAGACTATTACGGGGACCAATACAGTCATGGTGGACAA
GGTCCTCCAGAAGGCATGAACCAGCAATATTACCCTGATGGAAATTCACAGTATGGCCAACAGCAAGATG
CATACCAGGGACCACCTCCACAACAGGGATATCCACCCCAGCAGCAGCAGTACCCAGGGCAGCAAGGTTA
CCCAGGACAGCAGCAGGGCTACGGTCCTTCACAGGGTGGTCCAGGTCCTCAGTATCCTAACTACCCACAG
GGACAAGGTCAGCAGTATGGAGGATATAGACCAACACAGCCTGGACCACCACAGCCACCCCAGCAGAGGC
CTTATGGATATGACCAGATCATGCCCAAGAAGCCAGCAGAGGACGAAAATGATTCGAAGGGAGTGTCAGAAGC
ATCTGGCCCACAAAACGATGGGAAACAACTGCACCCCCCAGGAAAAGCAAATATTTCTGAGAAGATTAATAAG
AGATCTGGACCCAAAAGGGGGAAACATGCCTGGACCCACAGACTGCGTGAGAGAAAGCAGCTGGTGATTTATG
AAGAGATCAGTGACCCTGAGGAAGATGACGAGTAA (SEQ ID NO: 226)
* Included in Tables 1 and 2 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed in Table 1, or a portion thereof. Such nucleic acid molecules can have a function of the full-length nucleic acid as described further herein.
* Included in Tables 1 and 2 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequence listed in Table 1, or a portion thereof. Such polypeptides can have a function of the full-length polypeptide as described further herein.

### II. Subjects

In one embodiment, the subject for whom an agent that inhibits binding of a SS18-SSX fusion protein to an H2A K119Ub-marked nucleosome is administered, or whose predicted likelihood of efficacy of the agent for treating a cancer is determined, is a mammal (e.g., rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the present invention can be used to determine the responsiveness to the agent for treating a cancer. In one embodiment, the cancer is synovial sarcoma.

### III. Sample Collection, Preparation and Separation

In some embodiments, biomarker amount and/or activity measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment, evaluate a response to cancer therapy (e.g., an agent that inhibits binding of a SS18-SSX fusion protein to an H2A K119Ub-marked nucleosome), and/or evaluate a response to a combination cancer therapy (e.g., an agent that inhibits binding of a SS18-SSX fusion protein to an H2A K119Ub-marked nucleosome in combination of at least one immunotherapy). A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of cancer therapy. Post-treatment biomarker measurement can be made at any time after initiation of cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of cancer therapy, and even longer toward indefinitely for continued monitoring. Treatment can comprise cancer therapy, such as a therapeutic regimen comprising an agent that inhibits binding of a SS18-SSX fusion protein to an H2A K119Ub-marked nucleosome, or in combination with other cancer agents, such as with immune checkpoint inhibitors.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

One aspect of the present invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, NY, 1989).

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the present invention can be prepared by standard synthetic techniques, e.g., using an automated DNA synthesizer.

Moreover, a nucleic acid molecule of the present invention can comprise only a portion of a nucleic acid sequence, wherein the full length nucleic acid sequence comprises a marker of the present invention or which encodes a polypeptide corresponding to a marker of the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the present invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the present invention, e.g., complementary to the coding strand of a double-stranded cDNA molecule corresponding to a marker of the present invention or complementary to an mRNA sequence corresponding to a marker of the present invention. Accordingly, an antisense nucleic acid molecule of the present invention can hydrogen bond to (i.e. anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid.

Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an a-anomeric nucleic acid molecule. An a-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the present invention can be used to select a catalytic RNA having a specific ribonuclease activity from a pool of RNA molecules (see, e.g., Bartel and Szostak (1993) Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N. Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93:14670-14675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-3363. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-5988). PNA monomers are then coupled in a step-wise manner to produce a chimeric molecule with a 5′ PNA segment and a 3′ DNA segment (Finn et al. (1996) Nucleic Acids Res. 24:3357-3363). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the present invention are produced by recombinant DNA techniques. Alternative to recombinant expression, a polypeptide corresponding to a marker of the present invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the present invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the present invention. BLAST protein searches can be performed with the XBLAST program, score=50, wordlength=3 to obtain amino acid sequences homologous to a protein molecules of the present invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See world wide web ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The present invention also provides chimeric or fusion proteins corresponding to a biomarker protein. As used herein, a “chimeric protein” or “fusion protein” comprises all or part (preferably a biologically active part) of a polypeptide corresponding to a marker of the present invention operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide corresponding to a marker of the present invention is fused to the carboxyl terminus of GST sequences. Such fusion proteins can facilitate the purification of a recombinant polypeptide of the present invention.

In another embodiment, the fusion protein contains a heterologous signal sequence, immunoglobulin fusion protein, toxin, or other useful protein sequence. Chimeric and fusion proteins of the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide of the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan (1992) Proc. Natl. Acad. Sci. USA 89:7811-7815; Delgrave et al. 91993) Protein Engineering 6(3):327-331).

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, CA (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, MA) and pRIT5 (Pharmacia, Piscataway, NJ) which fuse glutathione S-transferase (GST), maltose E binding protein, or protein A, respectively, to the target recombinant protein.

Examples of suitable inducible non-fusion E. coli expression vectors include pTrc (Amann et al. (1988) Gene 69:301-315) and pET 11d (Studier et al., p. 60-89, In Gene Expression Technology: Methods in Enzymology vol. 185, Academic Press, San Diego, CA, 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, CA, 1990. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, CA), and pPicZ (Invitrogen Corp, San Diego, CA).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the present invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the a-fetoprotein promoter (Camper and Tilghman (1989) Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al. (1986) Trends in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number and/or Genomic Nucleic Acid Mutations

Methods of evaluating the copy number and/or genomic nucleic acid status (e.g., mutations) of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 of a biomarker is predictive of poorer outcome of treatment with the agent inhibiting binding of a SS18-SSX fusion protein to an H2A K119Ub-marked nucleosome.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, NY).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion.

Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to a modulator of T cell mediated cytotoxicity alone or in combination with an immunotherapy treatment. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (^{I12}In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a Ka of at most about 10⁻⁶M, 10⁻⁷M, 10⁻⁸M, 10⁻⁹M, 10⁻¹⁰M, 10⁻¹¹ M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify the SS18-SSX fusion protein/H2A K119Ub nucleosomes pathway proteins that are overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### VI. Cancer Therapies

The efficacy of a cancer therapy with an agent that inhibits binding of a SS18-SSX fusion protein to an H2A K119Ub-marked nucleosome is predicted according to biomarker presence, absence, amount and/or activity associated with a cancer in a subject according to the methods described herein. In one embodiment, such cancer therapy (e.g., an agent inhibiting binding of a SS18-SSX fusion protein to an H2A K119Ub-marked nucleosome) or combinations of therapies (e.g., an agent inhibiting binding of a SS18-SSX fusion protein to an H2A K119Ub-marked nucleosome, in combination with at least one immunotherapy) can be administered to a desired subject or once a subject is indicated as being a likely responder to cancer therapy (e.g., an agent inhibiting binding of a SS18-SSX fusion protein to an H2A K119Ub-marked nucleosome). In another embodiment, such cancer therapy (e.g., an agent inhibiting binding of a SS18-SSX fusion protein to an H2A K119Ub-marked nucleosome) can be avoided once a subject is indicated as not being a likely responder to the cancer therapy (e.g., an agent inhibiting binding of a SS18-SSX fusion protein to an H2A K119Ub-marked nucleosome) and an alternative treatment regimen, such as targeted and/or untargeted cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or without the agent inhibiting binding of a SS18-SSX fusion protein to an H2A K119Ub-marked nucleosome.

The term “targeted therapy” refers to administration of agents that selectively interact with a chosen biomolecule to thereby treat cancer. One example includes administration of an agent that inhibits binding of a SS18-SSX fusion protein to an H2A K119Ub-marked nucleosome. These agents block or otherwise reduce the interaction between a SS18-SSX fusion protein to an H2A K119Ub-marked nucleosome such that the activation of the SS18-SSX fusion protein target genes otherwise induced by the interaction is blocked or otherwise reduced. These agents may inhibit binding of a SS18-SSX fusion protein to an H2A K119Ub-marked nucleosome in a direct or indirect way.

Targeted therapy regarding the inhibition of immune checkpoint inhibitor is useful in combination with the methods of the present invention. The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). Inhibition of one or more immune checkpoint inhibitors can block or otherwise neutralize inhibitory signaling to thereby upregulate an immune response in order to more efficaciously treat cancer.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Similarly, agents and therapies other than immunotherapy or in combination thereof can be used with in combination with agents inhibiting binding of a SS18-SSX fusion protein to an H2A K119Ub-marked nucleosome to treat a cancer that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. No. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical--known as a photosensitizing agent--that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter--less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs encompassed by the present invention into the intended recipient. In one embodiment encompassed by the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment encompassed by the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the a- and 0-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide encompassed by the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### VII. Clinical Efficacy

Clinical efficacy can be measured by any method known in the art. For example, the response to a cancer therapy (e.g., an agent inhibiting binding of a SS18-SSX fusion protein to an H2A K119Ub-marked nucleosome), relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular agent that inhibits binding of a SS18-SSX fusion protein to an H2A K119Ub-marked nucleosome therapeutic regimen is at least 25%, 30%, 35%, 40%, 45%, 50%, 55%, 60%, 65%, 70%, 75%, 80%, 85%, or more.

Additional criteria for evaluating the response to cancer therapy (e.g., an agent that inhibits binding of a SS18-SSX fusion protein to an H2A K119Ub-marked nucleosome) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent inhibiting binding of a SS18-SSX fusion protein to an H2A K119Ub-marked nucleosome can be administered to a population of subjects and the outcome can be correlated to biomarker measurements that were determined prior to administration of any cancer therapy (e.g., an agent inhibiting binding of a SS18-SSX fusion protein to an H2A K119Ub-marked nucleosome). The outcome measurement may be pathologic response to therapy given in the neoadjuvant setting. Alternatively, outcome measures, such as overall survival and disease-free survival can be monitored over a period of time for subjects following cancer therapy (e.g., an agent inhibiting binding of a SS18-SSX fusion protein to an H2A K119Ub-marked nucleosome) for whom biomarker measurement values are known. In certain embodiments, the same doses of the agent inhibiting binding of a SS18-SSX fusion protein to an H2A K119Ub-marked nucleosome are administered to each subject. In related embodiments, the doses administered are standard doses known in the art for the agent inhibiting binding of a SS18-SSX fusion protein to an H2A K119Ub-marked nucleosome. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy (e.g., inhibiting binding of a SS18-SSX fusion protein to an H2A K119Ub-marked nucleosome) can be determined using methods such as those described in the Examples section.

### VIII. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell based assays and xenograft animal model assays. In one embodiment, the assays provide a method for identifying whether a cancer is likely to respond to cancer therapy (e.g., an agent inhibiting binding of a SS18-SSX fusion protein to an H2A K119Ub-marked nucleosome), such as in a human by using a xenograft animal model assay, and/or whether an agent can inhibit the growth of or kill a cancer cell that is unlikely to respond to cancer therapy (e.g., an agent inhibiting binding of a SS18-SSX fusion protein to an H2A K119Ub-marked nucleosome).

In one embodiment, an assay is a cell-based assay, comprising contacting a synovial sarcoma cancer cell with a test agent, and determining the ability of the test agent to decrease (1) binding of a SS18-SSX fusion protein to a H2A K119Ub nucleosome; (2) recruitment of a SS18-SSX fusion protein-bound BAF complex to a H2A K119Ub nucleosome; and/or (3) expression of at least one a SS18-SSX fusion protein target gene.

In another embodiment, an assay is a cell-free assay, comprising a) mixing a protein comprising a c-terminal basic region and a c-terminal acidic region of a SSX protein, and a H2A K119Ub nucleosome together; b) adding a test agent to the mixture; and c) determining the ability of the test agent to decrease binding of the protein to the H2A K119Ub nucleosome, and/or recruitment of the BAF complex to the H2A K119Ub nucleosome.

For example, in a direct binding assay, one protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product.

Determining the interaction between two molecules (e.g., a nucleosome and a SS18-SSX fusion protein) can be accomplished using standard binding or enzymatic analysis assays. These assays may include thermal shift assays (measure of variation of the melting temperature of the protein alone and in the presence of a molecule) (R. Zhang, F. Monsma, Curr. Opin. Drug Discov. Devel., 13 (4) (2010), pp. 389-402), SPR (surface plasmon resonance) (T. Neumann, et al. Curr. Top Med. Chem., 7 (16) (2007), pp. 1630-1642), FRET/BRET (Fluorescence or Bioluminescence Resonance Excitation Transfer) (A. L. Mattheyses, A. I. Marcus, Methods Mol. Biol., 1278 (2015), pp. 329-339; J. Bacart, et al. Biotechnol. J., 3 (3) (2008), pp. 311-324), Elisa (Enzyme-linked immunosorbent assay) (Z. Weng, Q. Zhao, Methods Mol. Biol., 1278 (2015), pp. 341-352), fluorescence polarization (Y. Du, Methods Mol. Biol., 1278 (2015), pp. 529-544), and Far western (U. Mahlknecht, O. G. Ottmann, D. Hoelzer J. Biotechnol., 88 (2) (2001), pp. 89-94) or other techniques. More sophisticated (and lower throughput) biophysical methods that provide structural or thermodynamic details of the molecule binding mode (using isothermal calorimetry (ITC), Nuclear Magnetic Resonance (NMR), and X-ray crystallography) may also be needed for further validation and characterization of potential hits.

Alternatively, high throughput cellular screens measuring the loss of interaction using reverse two hybrid or BRET may be used and offer the advantage of selecting only cell penetrable molecules (A. R. Horswill, S. N. Savinov, S. J. Benkovic Proc. Natl. Acad. Sci. USA, 101 (44) (2004), pp. 15591-15596; A. Hamdi, P. Colas Trends Pharmacol. Sci., 33 (2) (2012), pp. 109-118). The latter approaches require further validation to assess the “on target” effect. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies of the present invention can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to inhibit binding of a SS18-SSX fusion protein to an H2A K119Ub-marked nucleosomecan be accomplished by determining the ability of the test agent to modulate the activity of a polypeptide or other product that functions downstream or upstream of its position within the pathway. For example, it can be accomplished by measuring the activity of the downstream target genes of SS18-SSX fusion protein.

The present invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein in the context of a biological sample (e.g., blood, serum, cells, or tissue) to thereby determine whether an individual afflicted with a cancer is likely to respond to an agent inhibiting binding of a SS18-SSX fusion protein to an H2A K119Ub-marked nucleosome, such as in a cancer. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

Another aspect of the present invention pertains to monitoring the influence of agents (e.g., drugs, compounds, and small nucleic acid-based molecules) on the expression or activity of a biomarker described herein. These and other agents are described in further detail in the following sections.

The skilled artisan will also appreciate that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods encompassed by the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to cancer therapy (e.g., an agent that inhibits binding of a SS18-SSX fusion protein to an H2A K119Ub-marked nucleosome). In some embodiments, the present invention is useful for classifying a sample (e.g., from a subject) as associated with or at risk for responding to or not responding to cancer therapy (e.g., an agent that inhibits binding of a SS18-SSX fusion protein to an H2A K119Ub-marked nucleosome) using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to cancer therapy (e.g., an agent that inhibits binding of a SS18-SSX fusion protein to an H2A K119Ub-marked nucleosome) involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely cancer therapy (e.g., an agent that inhibits binding of a SS18-SSX fusion protein to an H2A K119Ub-marked nucleosome) responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to cancer therapy (e.g., an agent that inhibits binding of a SS18-SSX fusion protein to an H2A K119Ub-marked nucleosome), a biological sample from the subject during remission, or a biological sample from the subject during treatment for developing a cancer progressing despite cancer therapy (e.g., an agent that inhibits binding of a SS18-SSX fusion protein to an H2A K119Ub-marked nucleosome).

d. Prognostic Assays

The diagnostic methods described herein can furthermore be utilized to identify subjects having or at risk of developing a cancer that is likely or unlikely to be responsive to cancer therapy (e.g., an agent that inhibits binding of a SS18-SSX fusion protein to an H2A K119Ub-marked nucleosome). The assays described herein, such as the preceding diagnostic assays or the following assays, can be utilized to identify a subject having or at risk of developing a disorder associated with a misregulation of the amount or activity of at least one biomarker described in, such as in cancer. Alternatively, the prognostic assays can be utilized to identify a subject having or at risk for developing a disorder associated with a misregulation of the at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

The therapeutic compositions described herein, such as the agent that inhibits binding of a SS18-SSX fusion protein to an H2A K119Ub-marked nucleosome, can be used in a variety of in vitro and in vivo therapeutic applications using the formulations and/or combinations described herein. In one embodiment, the therapeutic agents can be used to treat cancers determined to be responsive thereto. For example, single or multiple agents that inhibit binding of a SS18-SSX fusion protein to an H2A K119Ub-marked nucleosome can be used to treat cancers in subjects identified as likely responders thereto.

Treatment methods of the present invention involve contacting a cell, such as a cancer cell with an agent that inhibits binding of a SS18-SSX fusion protein to an H2A K119Ub-marked nucleosome. An agent that inhibits binding of a SS18-SSX fusion protein to an H2A K119Ub-marked nucleosome can be an agent as described herein, such as a small molecule, a nucleic acid, a polypeptide, an antibody, or a peptidomimetic. In one embodiment, the agent binds to H2A K119Ub-marked nucleosomes or the SS18-SSX fusion protein at the interaction interface between the H2A K119Ub-marked nucleosomes and the SS18-SSX fusion protein, thereby blocking or competing with the H2A K119Ub-marked nucleosomes and the SS18-SSX fusion protein interaction formation. For example, the agent may bind to the basic region (e.g., the RLR motif) and/or the acidic region of the SS18-SSX fusion protein. The agent may bind to the acidic patch or the H2A K119Ub mark of the H2A K119Ub-marked neucleosomes. In another embodiment, the agent binds to another site of the H2A K119Ub-marked nucleosomes or the the SS18-SSX fusion protein and capable of inducing a conformational change leading to a loss of interaction with the targeted partner. In yet another embodiment, the agent inhibits the function or activity of a domain or a site of the H2A K119Ub-marked nucleosomes or the SS18-SSX fusion protein that is necessary for the H2A K119Ub-marked nucleosomes and the SS18-SSX fusion protein interaction formation. In still another embodiment, the agent inhibits the H2A ubiquitination of neucleosomes, induces deletion or mutation of the acidic patch of the H2A K119Ub-marked nucleosomes, and/or induces deletion or mutation of the basic region (e.g., RLR motif) of the SS18-SSX fusion protein itself, thus breaking the H2A K119Ub-marked nucleosomes and the SS18-SSX fusion protein interaction. In one embodiment, the agent inhibits ubiquitin ligase activity of a PRC1 complex. For example, the agent may reduces expression, copy number, and/or ubiquitin ligase activity of RING1A and/or RING1B. In another embodiment, the agent is a CRISPR/Cas9 reagent that targets the critical residues on the SS18-SSX fusion protein or the H2A K119Ub-marked nucleosomes important for the SS18-SSX fusion protein and the H2A K119Ub-marked nucleosomes interaction, which include but are no tlimited to the critical residues identified in the examples herein.

These treatment methods can be performed in vitro (e.g., by contacting the cell with the agent) or, alternatively, by contacting an agent with cells in vivo (e.g., by administering the agent to a subject). As such, the present invention provides methods useful for treating an individual afflicted with a condition that would benefit from a decreased activity of SS18-SSX target genes by inhibiting binding of a SS18-SSX fusion protein to an H2A K119Ub-marked nucleosome, such as a cancer like synovial sarcoma. In one embodiment, the method involves administering an agent (e.g., an agent identified by a screening assay described herein), or combination of agents that inhibit SS18-SSX target genes expression or activity.

In addition, these inhibitory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

### IX. Isolated Modified Protein Complexes

The present invention relates, in part, to an isolated modified protein complex selected from the group consisting of protein complexes listed in Table 3, wherein the isolated modified protein complex comprises at least one subunit that is modified.

In certain embodiments, at least one subunit of a complex encompassed by the present invention is a homolog, a derivative, e.g., a functionally active derivative, a fragment, e.g., a functionally active fragment, of a protein subunit of a complex encompassed by the present invention. In certain embodiments encompassed by the present invention, a homolog, derivative or fragment of a protein subunit of a complex encompassed by the present invention is still capable of forming a complex with the other subunit(s). Complex-formation can be tested by any method known to the skilled artisan. Such methods include, but are not limited to, non-denaturing PAGE, FRET, and Fluorescence Polarization Assay.

Homologs (e.g., nucleic acids encoding subunit proteins from other species) or other related sequences (e.g., paralogs) which are members of a native cellular protein complex can be identified and obtained by low, moderate or high stringency hybridization with all or a portion of the particular nucleic acid sequence as a probe, using methods well known in the art for nucleic acid hybridization and cloning.

Exemplary moderately stringent hybridization conditions are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 μM NaHPO₄, 7% sodium dodecyl sulfate (SDS), 1 mM EDTA at 65° C., and washing in 0.1×SSC/0.1% SDS at 68° C. (Ausubel et al., eds., 1989, Current Protocols in Molecular Biology, Vol. I, Green Publishing Associates, Inc., and John Wiley & sons, Inc., New York, at p.2.10.3). Other conditions of high stringency which may be used are well known in the art. Exemplary low stringency hybridization conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 μg/ml denatured salmon sperm DNA, and 1 0% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

In certain embodiments, a homolog of a subunit binds to the same proteins to which the subunit binds. In certain, more specific embodiments, a homolog of a subunit binds to the same proteins to which the subunit binds wherein the binding affinity between the homolog and the binding partner of the subunit is at least 10%, at least 20%, at least 30%, at least 40%, at least 50%, at least 60%, at least 70%, at least 80%, at least 90%, at least 95% or at least 98% of the binding affinity between the subunit and the binding partner. Binding affinities between proteins can be determined by any method known to the skilled artisan.

In certain embodiments, a fragment of a protein subunit of the complex consists of at least 6 (continuous) amino acids, of at least 10, at least 20 amino acids, at least 30 amino acids, at least 40 amino acids, at least 50 amino acids, at least 75 amino acids, at least 100 amino acids, at least 150 amino acids, at least 200 amino acids, at least 250 amino acids, at least 300 amino acids, at least 400 amino acids, or at least 500 amino acids of the protein subunit of the naturally occurring protein complex. In specific embodiments. Such fragments are not larger than 40 amino acids, 50 amino acids, 75 amino acids, 100 amino acids, 150 amino acids, 200 amino acids, 250 amino acids, 300 amino acids, 400 amino acids, or than 500 amino acids. In more specific embodiments, the functional fragment is capable of forming a complex encompassed by the present invention, i.e., the fragment can still bind to at least one other protein subunit to form a complex encompassed by the present invention. In one embodiment, the fragment of the subunit comprises the basic region and/or the acidic region of a SSX protein. In another embodiment, the fragment of the subunit comprises c-terminal 34 amino acids (aa155-188) of a SSX protein. In still another embodiment, the fragment of the subunit comprises c-terminal 78 amino acids (aa 111-188) of a SSX protein. The SSX protein may be selected form the group comsisting of human SSX1, SSX2, SSX3, SSX4, SSX6, SSX7, SSX8, and SSX9. In yet another embodiment, the fragment of the subunit comprises the acidic patch of a nucleosome and/or the H2A K119 Ub mark.

Derivatives or analogs of subunit proteins include, but are not limited, to molecules comprising regions that are substantially homologous to the subunit proteins, in various embodiments, by at least 30%, 40%, 50%, 60%, 70%, 80%, 90% or 95% identity over an amino acid sequence of identical size or when compared to an aligned sequence in which the alignment is done by a computer homology program known in the art, or whose encoding nucleic acid is capable of hybridizing to a sequence encoding the subunit protein under stringent, moderately stringent, or nonstringent conditions.

Derivatives of a protein subunit include, but are not limited to, fusion proteins of a protein subunit of a complex encompassed by the present invention to a heterologous amino acid sequence, mutant forms of a protein subunit of a complex encompassed by the present invention, and chemically modified forms of a protein subunit of a complex encompassed by the present invention. In a specific embodiment, the functional derivative of a protein subunit of a complex encompassed by the present invention is capable of forming a complex encompassed by the present invention, i.e., the derivative can still bind to at least one other protein subunit to form a complex encompassed by the present invention.

In certain embodiments encompassed by the present invention, at least two subunits of a complex encompassed by the present invention are linked to each other via at least one covalent bond. A covalent bond between subunits of a complex encompassed by the present invention increases the stability of the complex encompassed by the present invention because it prevents the dissociation of the subunits. Any method known to the skilled artisan can be used to achieve a covalent bond between at least two subunits encompassed by the present invention.

In specific embodiments, covalent cross-links are introduced between adjacent subunits. Such cross-links can be between the side chains of amino acids at opposing sides of the dimer interface. Any functional groups of amino acid residues at the dimer interface in combination with suitable cross-linking agents can be used to create covalent bonds between the protein subunits at the dimer interface. Existing amino acids at the dimer interface can be used or, alternatively, suitable amino acids can be introduced by site-directed mutagenesis.

In exemplary embodiments, cysteine residues at opposing sides of the dimer interface are oxidized to form disulfide bonds. See, e.g., Reznik et al., (1996) Nat Bio Technol 14:1007-1011, at page 1008. 1,3-dibromoacetone can also be used to create an irreversible covalent bond between two sulfhydryl groups at the dimer interface. In certain other embodiments, lysine residues at the dimer inter face are used to create a covalent bond between the protein subunits of the complex. Crosslinkers that can be used to create covalent bonds between the epsilon amino groups of lysine residues are, e.g., but are not limited to, bis(sulfosuccinimidyl)suberate; dimethyladipimidate-2HD1; disuccinimidyl glutarate; N-hydroxysuccinimidyl 2,3-dibromoproprionate.

In other specific embodiments, two or more interacting subunits, or homologues, derivatives or fragments thereof, are directly fused together, or covalently linked together through a peptide linker, forming a hybrid protein having a single unbranched polypeptide chain. Thus, the protein complex may be formed by “intramolecular interactions between two portions of the hybrid protein. In still another embodiment, at least one of the fused or linked interacting subunit in this protein complex is a homologue, derivative or fragment of a native protein.

In specific embodiments, at least one subunit, or a homologue, derivative or fragment thereof, may be expressed as fusion or chimeric protein comprising the subunit, homologue, derivative or fragment, joined via a peptide bond to a heterologous amino acid sequence.

As used herein, a “chimeric protein” or “fusion protein” comprises all or part (preferably a biologically active part) of a polypeptide corresponding to a subunit or a fragment, homologue or derivative thereof, operably linked to a heterologous polypeptide (i.e., a polypeptide other than the polypeptide corresponding to the subunit or a fragment, homologue or derivative thereof). Within the fusion protein, the term “operably linked” is intended to indicate that the polypeptide encompassed by the present invention and the heterologous polypeptide are fused in-frame to each other. The heterologous polypeptide can be fused to the amino-terminus or the carboxyl-terminus of the polypeptide encompassed by the present invention.

In one embodiment, the heterologous amino acid sequence comprises an affinity tag that can be used for affinity purification. In another embodiment, the heterologous amino acid sequence includes a fluorescent label. In still another embodiment, the fusion protein contains a heterologous signal sequence, immunoglobulin fusion protein, toxin, or other useful protein sequences.

A variety of peptide tags known in the art may be used to generate fusion proteins of the protein subunits of a complex encompassed by the present invention, such as but not limited to the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST: Smith, 1993, Methods Mol. Cell Bio. 4:220-229), the E. coli maltose binding protein (Guanetal., 1987, Gene 67:21-30), and various cellulose binding domains (U. S. Pat. Nos. 5,496,934: 5,202,247; 5,137,819; Tomme et al., 1994, Protein Eng. 7:117-123), etc.

One possible peptide tags are short amino acid sequences to which monoclonal antibodies are available, such as but not limited to the following well known examples, the FLAG epitope, the myc epitope at amino acids 408-439, the influenza virus hemaglutinin (HA) epitope. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

In certain embodiments, a combination of different peptide tags is used for the purification of the protein subunits of a complex encompassed by the present invention or for the purification of a complex. In certain embodiments, at least one subunit has at least two peptide tags, e.g., a FLAG tag and a His tag. The different tags can be fused together or can be fused in different positions to the protein subunit. In the purification procedure, the different peptide tags are used subsequently or concurrently for purification. In certain embodiments, at least two different subunits are fused to a peptide tag, wherein the peptide tags of the two subunits can be identical or different. Using different tagged subunits for the purification of the complex ensures that only complex will be purified and minimizes the amount of uncomplexed protein subunits, such as monomers or homodimers.

Various leader sequences known in the art can be used for the efficient secretion of a protein subunit of a complex encompassed by the present invention from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981. Proc. Natl. Acad. Sci. 78:5812-5816).

DNA sequences encoding desired peptide tag or leader peptide which are known or readily available from libraries or commercial suppliers are suitable in the practice of this invention.

In certain embodiments, the protein subunits of a complex encompassed by the present invention are derived from the same species. In more specific embodiments, the protein subunits are all derived from human. In another specific embodiment, the protein subunits are all derived from a mammal.

In certain other embodiments, the protein subunits of a complex encompassed by the present invention are derived from a non-human species, such as, but not limited to, cow, pig, horse, cat, dog, rat, mouse, a primate (e.g., a chimpanzee, a monkey Such as a cynomolgous monkey). In certain embodiments, one or more subunits are derived from human and the other subunits are derived from a mammal other than a human to give rise to chimeric complexes.

Included within the scope encompassed by the present invention is an isolated modified protein complex in which the subunits, or homologs, derivatives, or fragments thereof, are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH4, acetylation, formylation, oxidation, reduction, metabolic synthesis in the presence of tunicamycin, etc. In still another embodiment, the protein sequences are modified to have a heterofunctional reagent; such heterofunctional reagents can be used to crosslink the members of the complex.

The protein complexes encompassed by the present invention can also be in a modified form. For example, an antibody selectively immunoreactive with the protein complex can be bound to the protein complex. In another example, a non-antibody modulator capable of enhancing the interaction between the interacting partners in the protein complex may be included.

The above-described protein complexes may further include any additional components, e.g., other proteins, nucleic acids, lipid molecules, monosaccharides or polysaccharides, ions, etc.

TABLE 3

| Protein complex | Subunits of the protein complex |
|---|---|
| SS18-SSX | Subunit_1: SMARCC1 or SMARCC2 |
| BAF-NCP complex | Subunit_2: SMARCC1 or SMARCC2 |
| | Subunit_3: SMARCD1, SMARCD2, or SMARCD3 |
| | Subunit_4: SS18-SSX fusion protein |
| | Subunit_5: SMARCE1 |
| | Subunit_6: ARID1A or ARID1B |
| | Subunit_7: DPF1, DPF2, or DPF3 |
| | Subunit_8: ACTL6A |
| | Subunit_9: β-Actin |
| | Subunit_10: BCL7A, BCL7B, or BCL7C |
| | Subunit_11: SMARCA2 or SMARCA4 |
| | Subunit_12: H2A (with K119 Ub) |
| | Subunit_13: H2B |
| | Subunit_14: H3 |
| | Subunit_15: H4 |

### X. Pharmaceutical Compositions

In another aspect, the present invention provides pharmaceutically acceptable compositions which comprise a therapeutically-effective amount of an agent that inhibits binding of a SS18-SSX fusion protein to an H2A K119Ub-marked nucleosome, formulated together with one or more pharmaceutically acceptable carriers (additives) and/or diluents.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity. These salts can be prepared in situ during the final isolation and purification of the respiration uncoupling agents, or by separately reacting a purified respiration uncoupling agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression. These salts can likewise be prepared in situ during the final isolation and purification of the respiration uncoupling agents, or by separately reacting the purified respiration uncoupling agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a respiration uncoupling agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a respiration uncoupling agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more respiration uncoupling agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions encompassed by the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the respiration uncoupling agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

Other embodiments of the present invention are described in the following Examples. The present invention is further illustrated by the following examples which should not be construed as further limiting.

### EXAMPLES

### Example 1: Materials and Methods for Examples 2-5

a. Cell Lines and Cell Culture

The two synovial sarcoma cell lines, Aska and SYO1, were generous gifts from Kazuyuki Itoh, Norifumi Naka, and Satoshi Takenaka (Osaka University, Japan) and Akira Kawai (National Cancer Center Hospital, Japan), respectively. The CRL7250 human fibroblast cell line was obtained from Drs. Berkeley Gryder and Javed Khan (National Cancer Institute, Bethesda, MD). The HEK293T cell line was purchased ATCC (CRL-3216). Each cell line was cultured using standard protocols in DMEM medium (Gibco) supplemented with 10-20% fetal bovine serum, 1% Glutamax (Gibco), 1% Sodium Pyruvate (Gibco) and 1% Penicillin-Streptomycin (Gibco) and grown in a humidified incubator at 37° C. with 5% CO₂.

b. Stable Gene Expression and shRNA Knockdown Constructs

Constitutive expression of SS18 wild-type (SS18), SS18-SSX1 (SS18-SSX1) and SS18-SSX1 mutations with HA or V5 N-terminus tag was obtained using an EF1alpha-driven expression vector (modified from Clonetech, dual Promoter EF-1a-MCS-PGK-Puro or EF-la-MCS-PGK-Blast) expressed in cells by lentiviral infection and selected with puromycin (2 μg/mL) or blasticidin (10 μg/mL). Constitutive expression of shRNA hairpins targeting the 3′UTR region of SSX of the SS18-SSX fusion (5′-CAGTCACTGACAGTTAATAAA-3′ (SEQ ID NO: 227)) or a scramble non-targeting control (5′-CCTAAGGTTAAGTCGCCCTCGCTCGAGCGAGGGCGACTTAACCTTAGG-3′ (SEQ ID NO: 228)) was obtained using lentiviral infection of the pLKO.1 vector with puromycin (2 μg/mL) selection.

c. Lentivirus Generation and Harvesting

Lentivirus production was obtained from PEI (Polysciences) transfection of HEK293T LentiX™ cells (Clontech) with co-transfection of the packaging vectors pspax2 and pMD2.G along with the gene delivery vector. Viral supernatants were collected 72 hours after transfection, underwent ultracentrifugation at 20,000 rpm for 2.5 hr at 4° C. to concentrate, and then virus pellets were resuspended in PBS. For infection, the viral pellets were added to cells in a drop wise manner in the presence of polybrene (10 μg/mL). After 48 hours, the media containing the lentivirus was replaced and infected cells were selected by addition of puromycin (2 μg/mL) or blasticidin (10 μg/mL).

d. Western Blot Analysis

Detection of proteins by western blot (WB) analysis was achieved using standard protocols with primary antibodies (Table 4). Samples were separated on 4-12% Bis-Tris SDS PAGE gel (Invitrogen) and transferred to PVDF membrane. The membranes were then blocked in 5% milk and incubated with primary antibody in PBST over night at 4° C. Following incubation with the primary antibody, membranes were washed 3× in PBST, incubated with IRDye® (LI-COR Biosciences) secondary antibodies for 3 hours, washed 3× in PBST with a final PBS wash, and then visualized by the LI-COR Odyssey® Imaging System (LI-COR Biosciences).

TABLE 4
Description and characterization of antibodies used herein.

| Protein | Clone | Lot # | Catelog ID | Supplier | Application |
|---|---|---|---|---|---|
| BRG1 | G-7 | G0115 | sc-17796 | Santa Cruz | Immunoblotting |
| BRG1 | D1Q7F | 1 | 49360S | Cell Signaling Technology | Immunoprecipitation |
| SMARCB1/BAF47 | A-5 | K0515 | sc-166165 | Santa Cruz | Immunoblotting |
| SMARCC1/BAF155 | H-76 | | sc-10756 | Cell Signaling Technology | Immunoblotting |
| SS18 | D6I4Z | 1 | 21792S | Cell Signaling Technology | Immunoblotting & ChIP |
| RING1B | D22F2 | | 5694 | Cell Signaling Technology | Immunoblotting & ChIP |
| H2AK119Ub | D27C4 | | 8240 | Cell Signaling Technology | Immunoblotting & ChIP |
| H3 | | GR135489-1 | ab1791 | Abcam | Immunoblotting |
| GAPDH | G-9 | K2316 | sc-365062 | Santa Cruz | Immunoblotting |
| V5 tag | | 1805125 | P/N-46-0705 | Thermo Fisher Scientific | Immunoblotting |
| V5 tag | D3H8Q | 4 | 13202S | Cell Signaling Technology | ChIP |
| GFP | | | A-11120 | Thermo Fisher Scientific | Immunoblotting |
| GST | | | G7781 | Sigma-Aldrich | Immunoblotting |
| HA | | | Ab9110 | Abcam | Immunoblotting |
| MBP | | | E8032 | New England Biolabs | Immunoblotting |

e. Cell Lysate Collection

Whole cell extractions (WCE) were obtained by washing harvested cell pellets with PBS pH 7.4, resuspending in whole cell lysis buffer (PBS pH 7.4 and 1% SDS) and then heating for three minutes at 95° C. Lysates were sonicated until fully liquid. Nuclear extractions (NE) were obtained by suspending the harvested cells in Buffer 0 (50 mM Tris pH 7.5, 0.1% NP-40, 1 mM EDTA, 1 mM MgCl2 with protease inhibitor (Roche, C756U27), 1 mM DTT and 1 mM phenylmethylsulfonyl fluoride (PMSF)), centrifuging at 5,000 rpm for 5 minutes at 4° C., and discarding the supernatant. The pellet (nuclei) were resuspended in EB300 (50 mM Tris pH 7.5, 0.1% NP-40, 1 mM EDTA, 1 mM MgCl2, 300 mM NaCl with protease inhibitor cocktail (Roche, C756U27), 1 mM DTT and 1 mM phenylmethylsulfonyl fluoride (PMSF)), vortexed, incubated on ice, centrifuged at 15,000 rpm for 10 minutes at 4° C. and supernatant containing the nuclear extract collected.

f. Co-Immunoprecipitations

Nuclear extracts were quantified by Bradford assay and 150-200 μg of protein was incubated with 2 μg of antibody in Buffer EB300 (50 mM Tris pH 7.5, 0.1% NP-40, 1 mM EDTA, 1 mM MgCl2, 150 mM NaCl with protease inhibitor (Roche, C756U27), 1 mM DTT and 1 mM phenylmethylsulfonyl fluoride (PMSF)) overnight at 4° C. Each sample was then incubated with Protein G Dynabeads® (Thermo Scientific) for 2-3 hours. Beads were washed three times with Buffer EB300 followed by elution with 20 μL of elution buffer (NuPage™ LDS buffer (2×) (Life Technologies) containing 100 mM DTT and water).

g. Cell Proliferation Assay

To measure cell proliferation following lentiviral infection, 2.5×10⁴ cells per well were seeded in 12-well plates following 48-hour exposure to lentivirus and 5-day selection with puromycin or blasticidin, with Day 7 denoting the day cells were plated after infection and selection. The cell viability in three wells was then measured using a Vi-CELL™ Cell Counter (Beckman, Brea, CA) every 72 hours.

h. Differential Salt Extraction

Following collection of 5.0×10⁷ cells, cells were resuspended in elution 0 buffer (50 mM Tris-HCl pH 7.5, 1 mM EDTA, 0.1% NP40 with protease inhibitor mixture (Roche, C756U27) and 1 mM PMSF), incubated on ice for 5 minutes, and pelleted by centrifugation. The supernatant was collected (0 mM fraction), and the cell pellet was resuspended in elution 150 buffer (50 mM Tris-HCl pH 7.5, 150 mM NaCl) 1 mM EDTA, 0.1% NP40 with protease inhibitor mixture (Roche, C756U27) and 1 mM PMSF) and vortexed. This process was repeated sequentially with elution 300 buffer, elution 500 buffer, and elution 1000 buffer that contained increasing concentrations of NaCl in order to obtain 0, 150, 300, 500, and 1,000 mM NaCl soluble fractions. Each of these soluble fractions, along with a total sample (5×10⁶ cells in elution buffer) and the chromatin pellet (non-soluble material remaining following extraction with 1000 mM NaCl) fractions, was denatured in SDS to a final concentration of 1%, protein quantified by Pierce™ BCA Protein Assay Kit (Thermo Fisher Scientific), and analyzed (1.5 μg of protein) by immunoblot.

i. Purification of mSWI/SNF (BAF) Complexes

Stable HEK293T cell lines expressing by lentiviral infection HA-SS18 WT or HA-SS18-SSX1 were grown in 150 mm dishes. Complexes were purified using methods previously described with a few modifications (Mashtalir et al. (2014) Mol. Cell 54:392-406). Confluent plates were scraped to remove cells and cells were washed with PBS. Cell suspension was spun down by centrifugation at 3000 rpm for 5 minutes at 4° C. and pellets were resuspended in hypotonic buffer (10 mM Tris HCl pH 7.5, 10 mM KCl, 1.5 mM MgCL2, 1 mM DTT, 1 mM PMSF) and incubated on ice for 5 minutes. Following incubation, cell suspension was spun down by centrifugation at 5000 rpm for 5 minutes at 4° C., and pellets were resuspended in 5× volume of fresh hypotonic buffer (with protease inhibitor cocktail, Roche C756U27) and then cells were homogenized using a Dounce homogenizer (glass). Cell suspension was layered onto hypotonic buffer sucrose cushion made with 30% sucrose w/v, spun down by centrifugation at 5000 rpm for 1 hour at 4° C. followed by removal of the cytosol-containing layer. The nuclei containing pellets were resuspended in high salt buffer (50 mM Tris HCl pH 7.5, 300 mM KCl, 1 mM MgCL2, 1 mM EDTA, 1 mM, 1% NP40, 1 mM DTT, 1 mM PMSF and protease inhibitor cocktail) and then the homogenate rotated for 1 hour at 4° C. Homogenates were then spun down by centrifugation at 20,000 rpm for 1 hour at 4° C. in a SW32Ti rotor (Beckman Coulter). The soluble proteins, consisting of the nuclear extract (NE) fraction, was separated from the insoluble chromatin pellet, consisting of the chromatin (CHR) fraction. The chromatin pellet was further solubilized by treatment with Benzonase® (Sigma Aldrich) for 30 minutes and subsequently additional KCl was added to final concentration of 700 mM (50 mM Tris HCl pH 7.5, 700 mM KCl, 1 mM MgCL2, 1 mM EDTA, 1 mM, 1% NP40, 1 mM DTT, 1 mM PMSF and protease inhibitor cocktail), and sonicated 3 times for 30 seconds with 5-minute intervals. The solubilized chromatin fraction was then spun down by centrifugation at 20,000 rpm for 1 hour at 4° C. in a SW32Ti rotor (Beckman Coulter) and supernatant was collected. The collected nuclear extract and chromatin fractions were filtered with a 0.45 μm filter and rotated overnight at 4° C. with HA magnetic resin. HA beads were washed in high salt buffer and eluted with 1 mg/mL of HA peptide for 4 times at durations of 1.5 hour each. Eluted proteins were then subjected to density gradient centrifugation or dialysis.

### J. Colloidal Blue and Silver Stain

HA-SS18 WT and HA-SS18-SSX1 mSWI/SNF complexes were purified via HA-epitope-dependent complex purification. Importantly, for FIG. 1A, the same number of cells were used for both HA-SS18 WT and HA-SS18-SSX expressing cells, and nuclear material from both cell lines was split into NE and CHR fractions, representing an equal total amount of complexes in the nucleus. Hence, equal input/output loading by volume was achieved. Samples were run on a 4-12% Bis-Tris SDS PAGE gel, stained using Colloidal blue kit or SilverQuest™ Silver Staining Kit (Invitrogen), and imaged using LI-COR Odyssey® Imaging System (LI-COR Biosciences) or Epson-Perfection V600 Photo scanner, respectibly.

k. Density Sedimentation Gradients

Purified protein complexes were added to the top of a linear, 11 ml 10%-30% glycerol gradients containing 25 mM HEPES pH 7.9, 0.1 mM EDTA, 12.5 mM MgCl2, 100 mM KCl with 1 mM DTT and protease inhibitors (Roche, C756U27). Gradient tubes were placed into SW41 rotor (Beckman Coulter) and spun by centrifugation at 40000 rpm for 16 hours at 4° C. Fractions of 550 μL volume were collected sequentially from the top of the gradient. 100 μL of each fraction was concentrated with 10 μL of Strataclean beads (Agilent Technologies, 400714), loaded and run on a SDS-PAGE gel, and then analyzed by SYPRO® Ruby Protein Gel Stain (Thermo Fisher Scientific) and scanned using Typhoon™ FLA 9500 scanner.

l. Mass Spectrometry Proteomics Analysis of Purified Complexes

Equal amounts of purified HA-SS18 WT and HA-SS18-SSX1 complexes were loaded onto SDS-PAGE gels from both the nuclear extract (NE) and chromatin (CHR) fractions. Samples were migrated into the gel for a length of 2 cm, gels were stained with colloidal blue stain and protein bands were excised for protein detection by mass spectrometry. The samples were then prepared and data were analyzed by the Taplin Biological Mass Spectrometry Facility directed by Dr. Steven Gygi (Harvard Medical School).

m. Protein and Peptide Pull Downs

Recombinant purified proteins with affinity tags (MBP or GST) or biotinylated peptides were purified using magnetic beads (Maltose, glutathione or streptavidin respectably) by incubation in EB150 buffer (50 mM Tris-HCl pH 7.5, 150 mM NaCl) 1 mM EDTA, 0.1% NP40 with protease inhibitor mixture (Roche, C756U27) and 1 mM PMSF) at 4° C. overnight. The flow through was removed, the immobilized bait was incubated with 1-2 μg of purified mammalian mono-nucleosomes from HEK293T cells, recombinant mono-nucleosomes (EpiCypher, 16-0006), recombinant H2AK119Ub mono-nucleosomes (EpiCypher, 16-0020) or recombinant protein for 3 hours at 4° C., and the beads were washed 3× with EB150 buffer and then eluted in 2×LDS with 200 mM DTT with heating at 95° C. for 5 minutes. The pull downs were then visualized by immunoblot analysis or colloidal blue staining.

n. Peptide Competition Experiments

The peptide competition experiments were set up in a similar manner as the peptide pull down experiments with the following exceptions: SSX1 (aa 55-78) or SMARCB1-CC (aa 351-385) biotin-labeled peptides at 10 μM in EB150 were bound to Streptavidin Dynabeads® (Pierce Streptavidin Magnetic Beads, Thermo Scientific) in parallel to 1-2 μg of mononucleosomes incubated with LANA, SSX (aa 155-188) or SMARCB1-CC (aa 351-385) peptide (KE Biochem) at varying concentrations ranging from 0-30 μM overnight at 4° C. Beads were washed 3 times in EB150, and resuspended with the mononucleosome/LANA peptide solutions. The suspension was rotated for 3-5 hours at 4° C. The beads were washed 5 times in EB150, and eluted in Sample Buffer (2×LDS with 200 mM DTT) to load onto 10-20% Tricine gels.

o. Quantitative Targeted Mass Spectrometry

Mammalian mono-nucleosomes purified from MBP-SSX1 78aa pull downs along with representative input samples were prepared and analyzed by the targeted mass spectrometry pipeline described previously (Creech et al. (2015) Methods 72:57-64). Briefly, samples were prepared by histone extraction by acid precipitation followed by protein digestion from incubation with trypsin. To these prepared samples, synthesized isotopically labeled peptides of histone tails with numerous modifications were added at a known quantity. Each sample was then separated using a Proxeon EASY-nLC™ 1000 UHPLC system (Thermo Scientific) and detected with a Q Exactive™ mass spectrometer (Thermo Scientific). The fold change in abundance of each histone peptide from the input sample compared to the pull down was calculated from the light:heavy ratio in detected peak size.

p. Detection of Nucleosome Acidic Patch Interactions by Photocrosslinking

Details of the design and preparation of diazirine containing nucleosomes for photo-crosslinking studies were described elsewhere (Dao et al. (2019) Nat. Chem. Biol. doi:10.1038/s41589-019-0413-4). Briefly, diazirine-containing recombinant nucleosomes (0.5 uM) were incubated with biotinylated SSX peptides (12.5 uM) in binding buffer (20 mM HEPES, pH 7.9, 4 mM Tris, pH 7.5, 150 mM KCl, 10 mM MgCl2, 10% glycerol, and 0.02% (v/v) IGEPAL CA-630) at 30° C. for 30 mins, and cooled on ice for 5 mins. The reaction mixtures were then irradiated at 365 nm for 10 minutes. Reactions were then analyzed by western blotting employing IRDye® 800CW streptavidin on a LI-COR Odyssey® Infrared Imager. Additional details are found in Dao et al. (2019) Nat. Chem. Biol. doi:10.1038/s41589-019-0413-4.

q. Immunofluorescence

Immunofluorescent images were obtained as previously described (Daou et al. (2011) Pro. Nat. Acad. Sci. U.S.A 108:2747-2752). Following lentiviral infection and/or drug treatment, cells were prepared by fixation in 3% PFA-PBS and then were permeabilized with PBS 0.1% NP40. Following incubation with primary antibodies, the Anti-rabbit Alexa Fluor® 594 and Anti-mouse Alexa Fluor® 488 (Life Technologies) secondary antibodies were used for visualization. Staining with 4′,6-diamidino-2-phenylindole (DAPI) was used to visualize nuclei. Images were acquired using Zeiss Axio Imager Z2 microscope and images were processed using ImageJ program (NIH).

r. Fluorescent Recovery after Photobleaching (FRAP)

The FRAP experiments were carried out in the same manner as previously described (Carvalho et al. (2004) Dev. Cell 6:815-829). Briefly, HEK293T cells expressing GFP-SS18 WT or GFP-SS18-SSX1 by lentiviral infection or Aska cells co-expressing BRG1-Halo fusion with pLKO.1 shScramble control or shSSX were imaged to measure the mean fluorescence intensity of a defined nuclear region pre and post-photobleaching at 5 second intervals. The relative fluorescence intensity (RFI) for each image was calculated by normalizing the maximal difference in fluorescence intensity post-bleaching to 1. The ti/2 values and mobile fractions were determined using the software Prism (GraphPad Software) from >n=27-30 cells in each condition over two biological replicates.

s. Chromatin Immunoprecipitation (ChIP)

For chromatin immunoprecipitation (ChIP) experiments, prepared cells were harvested following 48 hours of lentiviral infection and 5 day selection (unless otherwise indicate) with puromycin or blasticidin. Capture of chromatin bound proteins was performed using standard protocols (Millipore, Billerica, MA). Briefly, cells were cross-linked with 1% formaldehyde for 10 minutes at 37° C., reaction was quenched by addition of 125 mM glycine for 5 min and then 5 (for synovial sarcoma cell lines) or 10 (for fibroblast cell lines) million fixed cells were used per experiment. Chromatin was fragmented by sonication with a Covaris E220 and the solubilized chromatin was incubated with a primary antibody overnight at 4° C. to form antibody-chromatin complexes. These complexes were incubated with Protein G-Dynabeads® (Thermo Scientific) for 3 hours at 4° C. Beads were then washed 3× and eluted. The samples then underwent crosslink reversal, treatment with RNase A (Roche), and treatment with proteinase K (Thermo Scientific) followed by DNA capture with AMPure beads (Beckman Coulter).

t. RNA Isolation from Cell Lines

Cells (1×10⁶) were collected following 48 hours of lentiviral infection and 5 days (7 days post-infection) of selection with puromycin or blasticidin for extraction of RNA for RNA-seq experiments. Samples for RNA-seq were prepared in biological duplicates (collected using independent production of lentivirus, infection, selection, and cell culture). Total RNA was collected using the RNeasy® Mini Kit (Qiagen) following homogenization of cell lysates using the QIAshredder (Qiagen).

u. Library Preparation and Sequencing for RNA and ChIP Samples

Library preparations for next-generation sequencing of RNA-seq samples were performed using the NEBNext® Poly(A) mRNA Magnetic Isolation Module (New England BioLabs) to purify mRNA from 1 μg of total RNA isolated from cells. Next, the isolated mRNA was used with the NEBNext® Ultra™ II Directional RNA Library Prep Kit for Illumina (New England BioLabs) to generate DNA. The DNA from these prepared RNA samples as well as the ChIP-seq samples were then prepared for sequencing using the NEBNext® Ultra™ II (New England BioLabs) to amplify and barcode each sample. The fragments sizes were determined using a D1000 ScreenTape system (Agilent) and the DNA quantified by Kapa Library Quantification Kit Illumina® Platforms (Kapa Biosystems). The samples were then diluted and loaded on a buffer cartridge for 75 bp single end sequencing on the NextSeq™ 500 system (Illumina).

v. Data Processing and Visualization for ChIP Samples

Alignment of ChIP-seq data was done using Bowtie2, version 2.1.0 (Langmead and Salzberg (2012) Nat. Meth. 9:357-359) and reads were mapped to the hg19 human reference genome, using the parameter -k 1.

To process the aligned data, peaks were called using MACS2 (Zhang et al. (2008) Genome Biol. 9:R137) version 2.1.0 against an input sample with a q=0.001 cutoff and broad peaks were called for each antibody in each cell line and condition. Those peaks that were mapped to unmappable chromosomes (any that were not chr1-22, X or Y) or were located in blacklisted regions of ENCODE were excluded. For downstream analysis of data, bam files were generated with duplicates removed using the samtools rmdup command and the -b option. All ChIP-seq tracks were obtained from the bedGraphToBigWig script (UCSC) using bedgraph files generated with MACS2 using the -B-SPMR options. ChIP-seq tracks were visualized using IGV version 2.4.16 (Broad Institute).

To identify peaks of BAF complex localization, the merged peak set for V5 in V5-SS18 WT and V5-SS18-SSX1 conditions was used with bedtools merge -d 2000 to cause neighboring broad peaks to be called as a single peak. Read counts across peak sets were determined by calling the Rsubread v1.26.1 bioconductor package function feature Counts( ) on bam files. Subsequently, these values were divided by the total number of mapped reads divided by one million to give a normalized value of RPM for each interval contained within the input bed.

HTSeq was used to calculate metagene read densities with fragment lengths of 200 bp to account for fragment size selection that occurs during sonication. Total read counts for each region was normalized by the number of mapped reads to calculate reads per million mapped reads. The metagene plots were created using mean read densities over all sites for each condition around the center of the peak. All ChIP-seq heatmaps were created using these same HTSeq read densities with sites were then ranked by mean ChIP-seq signal for the indicated antibody and condition. Heatmap visualization was obtained from Python matplotlib using a midpoint of 0.5 reads per million to set the threshold of visualization for the heatmap color scale.

w. Data Processing and Visualization for RNA Samples

STAR was used to determine RPM values for each sample. Significance was determined with the DESeq2 R package with input raw read counts obtained from Rsubread featureCounts against the hg19 refFlat annotation. Small RNA genes (MIR & SNO) were filtered out from the gene lists for all analyses. Genes with a significant change in expression were determined with a Bonferri-corrected p-value of less than 1e-5, a two-fold change in gene expression (|log 2FC|>1), and inclusion of expressed genes (RPKM≥1 in a minimum of one sample) to identify significantly changing genes. For visualization of RNA-seq data, heatmaps were generated by plotting the z-scores of RPKM values across each sample of the comparison conditions.

X. CRISPR-Cas9 and shRNA Synthetic Lethal Screening Data Analyses

CRISPR-Cas9 datasets (Avana-19Q3) were obtained from the Project Achilles Data Portal (available on the World Wide Web at depmap.org/portal/achilles/). Fitness (CERES) scores were extracted for each cell line and hierarchical clustering was performed using complete linkage and correlation as a distance measure. Heatmaps were generated using pheatmap in RStudio. DRIVE data is publicly available and can be downloaded from the Novartis DRIVE Data Portal (available on the World Wide Web at oncologynibr.shinyapps.io/drive/). Waterfall plots were generated using ggplot2 in RStudio.

y. Purification of Mammalian Mononucleosomes

Mammalian mononucleosomes were purified from HEK293T cells similar to as previously described (Mashtalir et al. (2014) Mol. Cell 54:392-406). Cells were scraped from plates, washed with cold PBS, and centrifuged at 5,000 rpm for 5 min at 4° C. Pellets were resuspended in hypotonic buffer (EBO: 50 mM Tris HCl, pH 7.5, 1 mM EDTA, 1 mM MgCl₂, 0.1% NP40 supplemented with 1 mM DTT, 1 mM PMSF, and protease inhibitor cocktail (Roche, C756U27) and incubated for 5 min on ice. The suspension was centrifuged at 5,000 rpm for 5 min at 4° C., and pellets were resuspended in 5 volumes of EB420 (EBO: 50 mM Tris HCl, pH 7.5, 420 mM NaCl, 1 mM MgCl2, 0.1% NP40 with supplemented with 1 mM DTT and 1 mM PMSF containing protease inhibitor cocktail (Roche, C756U27). Homogenate incubated on rotator for 1 hour at 4° C. The supernatant was then centrifuged at 20,000 rpm (30,000×g) for 1 hour at 4° C. using a SW32Ti rotor. Supernatant was then discarded and chromatin pellet was washed in MNAse buffer (20 mM Tris-HCl pH 7.5, 100 mM KCl, 2 mM MgCl₂, 1 mM CaCl₂, 0.3 μM sucrose, 0.1% NP-40, and protease inhibitor cocktail) three times. Following MNase treatment (3 U/mL for 30 min at room temperature, Sigma-Aldrich), the reaction was quenched with 5 mM of EGTA and 5 mM of EDTA. The samples were then centrifuged at 20,000×g for 1 hour at 4° C. to obtain the soluble chromatin fraction. Soluble chromatin fraction was loaded onto 10-30% glycerol gradient (Mashtalir et al. (2014) Mol. Cell 54:392-406) and fractions containing mononucleosomes were isolated and concentrated using centrifugal filter (Amicon, EMD Millipore).

z. Restriction Enzyme Accessibility Assay (REAA) Nucleosome Remodeling Assay

SMARCA4 (BRG1) levels of the ammonium sulfate nuclear extracts were normalized via BCA protein quantification and Silver Stain analyses for HA-SS18 and HA-SS18-SSX conditions. Protein was diluted for final reaction concentration of 150 μg/mL in REAA buffer (20 mM HEPES, pH 8.0, 50 mM KCl, 5 mM MgCl2) containing 0.1 mg/mL BSA, 1 mM DTT, 20 nM nucleosomes (EpiDyne Nucleosome Remodeling Assay Substrate ST601-GATC1, EpiCypher). The REAA mixture was incubated at 37° C. for 10 min, and reaction was initiated using 1-2 mM ATP (Ultrapure ATP, Promega) and 0.005 U/mL DpnJJ Restriction Enzyme (New England Biolabs). The REAA reaction mixture was quenched with 20-24 mM EDTA and placed on ice. Proteinase K (Ambion) was added at 100 mg/mL for 30-60 min, followed by either AMPure bead DNA purification and D1000 HS DNA ScreenTape Analysis (Agilent) or mixing with GelPilot® Loading Dye (QIAGEN) and loading onto 8% TBE gel (Novex 8% TBE Gels, Thermo Fisher). TBE gels were stained with either SYBR®-Safe (Invitrogen) or Syto®-60 Red Fluorescent Nucleic Acid Stain (Invitrogen), followed by imaging with UV light on an Alpha Innotech AlphaImager™ 2200 and/or with 652 nm light excitation on a Li-Cor Odyssey® CLx imaging system (LI-COR).

aa. Preparation of Peptides

Custom peptide sequences were prepared using standard synthesis techniques from KE Biochem. The peptides were confirmed to have >95% purity by HPLC and obtained as a white to off-white lyophilized powder. The powder was re-suspended in DMSO (Sigma) for use in experiments.

ab. Expression and Purification of Recombinant Proteins

DNA constructs of human SSX1 aa111-188 and related mutates in pGEX-6P2 expression vector were transformed in E. coli BL21 (DE3) cells and overexpressed in TB medium in the presence of 100 μg/ml of ampicillin. Cells were grown at 37° C. to an OD600 of 0.6, cooled to 17° C., induced with 500 μM isopropyl-1-thio-D-galactopyranoside (IPTG), incubated overnight at 17° C., collected by centrifugation, and stored at −80° C. For ¹³C- and ¹⁵N-labeled protein expression for NMR analysis, minimal media containing ¹³C-labeled glucose and ¹⁵N-labeled ammonium chloride was used for E. coli growth and protein expression, following an established protocol (Marley et al. (2001) J. Biomol. NMR 20:71-75). Cell pellets were resuspended in buffer A (25 mM HEPES, pH 7.5, 200 mM NaCl, 5% glycerol, and 0.5 mM TCEP) supplemented with 1 mM PMSF, lysed in a Microfluidizer (Microfluidics) and centrifuged at 16,000×g for 45 min. Glutathione sepharose beads (GE healthcare) were incubated with lysate supernatant for 90 min to captured GST-tagged proteins and washed with buffer A. Beads with bound protein were transferred to an FPLC-compatible column and the bound protein was washed with high salt buffer (buffer A containing 1M NaCl) followed by elution with buffer A supplemented with 15 mM glutathione (Sigma). Eluted protein fractions were collected, concentrated and purified by size exclusion chromatography using a Superdex® 75 10/300 column (GE healthcare) equilibrated with buffer A. Eluted protein was incubated with GST-3C protease at 4° C. overnight. Cleaved samples were incubated with a second round of glutathione beads to remove GST-3C and free GST, and desired protein product contained within the flow-through fractions was further purified by ion-exchange chromatography using mono-Q column (GE healthcare). Fractions containing the cleaved protein product were pooled, concentrated and stored at −80° C.

Ac. Peptide Hybridization Assay

IMR90 fibroblasts were grown on coverslips, washed with PBS and fixed using 100% ice-cold methanol for 3 minutes. Coverslips were then washed with IF wash buffer (PBS 0.1% NP40 1 mM Sodium azide) 3 times. Selected groups were treated with 200 ng/ml of recombinant USP2 catalytic domain (Boston biochem) for 1 hour. Coverslips were then washed 3 times with IF wash buffer and incubated with 2 μM of biotinylated peptides. Coverslips were subsequently washed 3 times with IF wash buffer and fixed in 3% PFA-PBS for 20 minutes. The rest of the procedure followed accordingly to standard IF protocol. In brief, following incubation with primary antibodies, the Anti-rabit Alexa Fluor® 594 and Streptavidin Alexa Fluor® 488 (Life Technologies) secondary antibodies/reagent were used for visualization of primary antibodies or biotinylated peptides. Staining with 4′,6-diamidino-2-phenylindole (DAPI) was used to visualize nuclei. Images were acquired using Zeiss Axio Imager Z2 microscope and images were processed using ImageJ program (NIH).

ad. NMR Structure Prediction

¹⁵N and ¹³C doubly-labeled C-terminal deletion mutant SSX1-7aa (aal11-181) protein were expressed from E. coli in M9 minimal medium containing ¹⁵NH₄Cl and ¹³C-glucose as the sole nitrogen and carbon sources. Non-uniformly-sampled (NUS) triple resonance experiments, HNCA, HN(CO)CA, HNCO, HN(CA)CO, HN(CA)CB, HN(COCA)CB, and C(CO)NH, using 0.33 mM ₁₅N/₁₃C-SSX1-7aa(aa 111-181) protein in PBS buffer, pH 6.5 with 10% D20, were performed at 15° C. on a 700 MHz Agilent DD2 spectrometer equipped with a cryogenic probe. The data were processed using NMRPipe (Delaglio et al. (1995) J. Biomol. NMR 6:277-293) and Iterative Soft Thresholding reconstruction approach (istHMS) (Hyberts et al. (2012) J. Biomol. NMR 52:315-327) and analyzed by CARA (Keller (2005) ETH). Backbone dihedral angle restraints and secondary structure predications based on assigned chemical shifts were obtained using the TALOS+ software (Shen et al. (2009) J. Biomol. NMR 44:213-223).

ae. Nuclear Extraction

Nuclear extracts for 293T V5-SS18WT and V5-SS18-SSX1 cells were prepared as described in Mashtalir et al. (2018) Cell 175:1272-1288. Specifically, cells were scraped from plates, washed with cold PBS, pelleted at 3,000 rpm for 5 min at 4° C., and resuspended in Buffer A hypotonic buffer (50 mM Hepes, pH 7.6, 25 mM KCl, 10% Glycerol, 0.1% NP-40, 0.05 mM EDTA, 5 mM MgCl2 supplemented with protease inhibitor (Roche), and 1 mM phenylmethylsulfonyl fluoride (PMSF)). Lysates were pelleted at 3,000 rpm for 5 min at 4 C. Supernatants were discarded, and nuclei were resuspended in Buffer C high salt buffer (10 mM Hepes, pH 7.6, 100 mM KCl, 10% Glycerol, 0.5 mM EDTA, 3 mM MgCl2 supplemented with protease inhibitor and 1 mM PMSF). Lysates were incubated at 4° C. at constant rotation. Lysates were then pelleted at 40,000×rpm for 1 hour at 4° C. Supernatants were collected, and mixed with (NH4)2SO4 at 300 mg/ml for 30 min. Samples were pelleted at 15,000 rpm for 30 minutes and supernatant was discarded. Protein concentrations were quantified via bicinchonic acid (BCA) assay (Pierce). Finally, samples were supplemented with 1 mM DTT.

af. ATPase Assays

ATPase consumption assays were performed using the ADP-Glo Kinase Assay kit (Promega). The same conditions as the REAA nucleosome remodeling assay described above were used. Following incubation with desired substrates for 40 min at 37° C., 1× volume of ADP-Glo Reagent was used to quench the reaction and incubated at RT for 40 min. 2× volume of the Kinase Detection Reagent was then added and incubated at RT for 1 h. Luminescence readout was recorded. Substrates used for this assay were purified recombinant mononucleosome (EpiDyne Nucleosome Remodeling Assay Substrate ST601-GATC1, EpiCypher, Cat #16-4101). Nuclear extract material was used at 150ug for each ARID1A-IP using ARID1A antibody (Cell Signaling, Cat #12354S).

### Example 2: SS18-SSX-Bound BAF Complexes Bind Chromatin with Uniquely High Affinity Via Stoichiometric Histone Binding

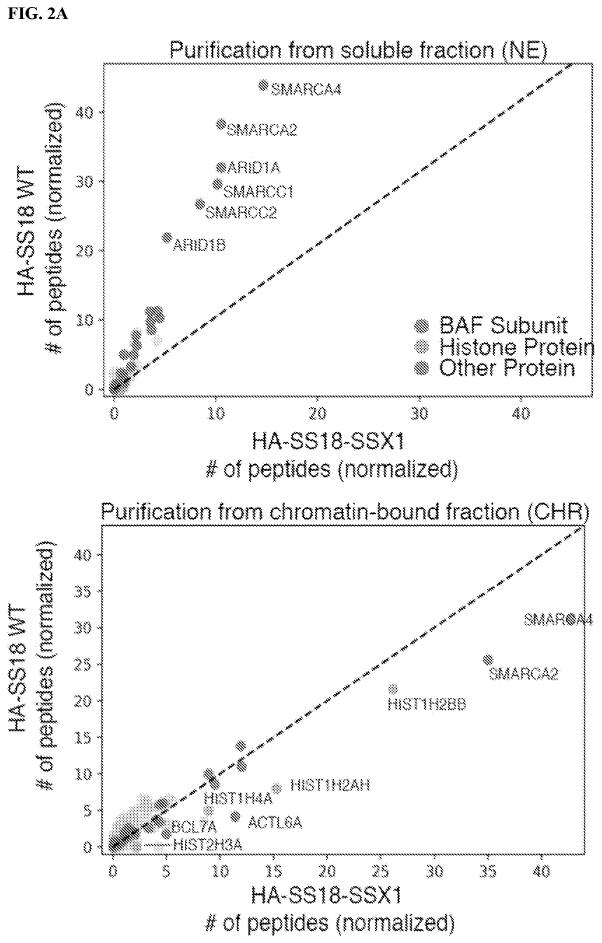

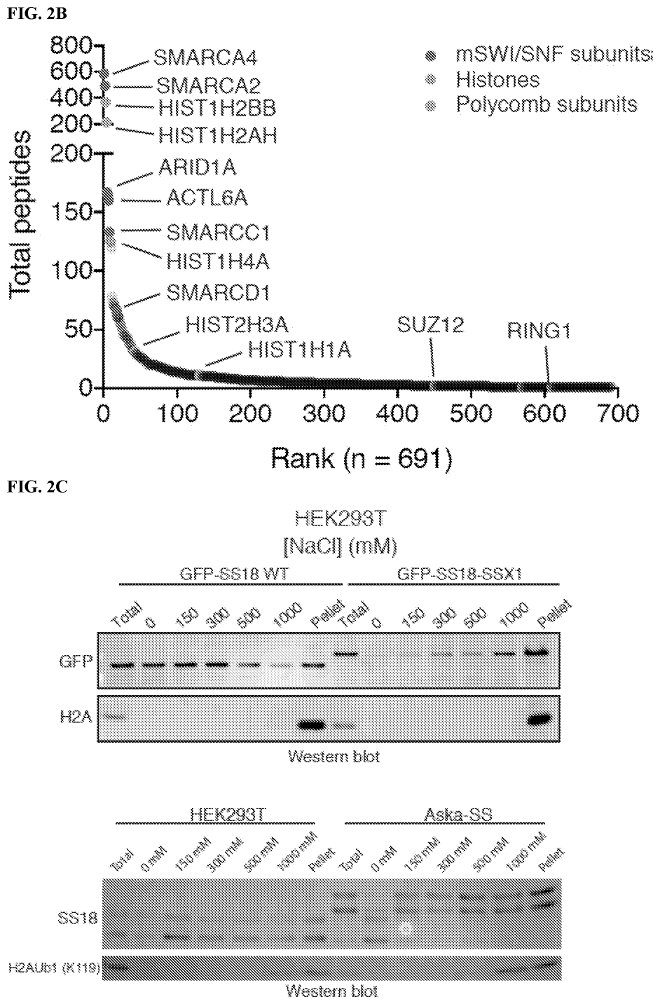

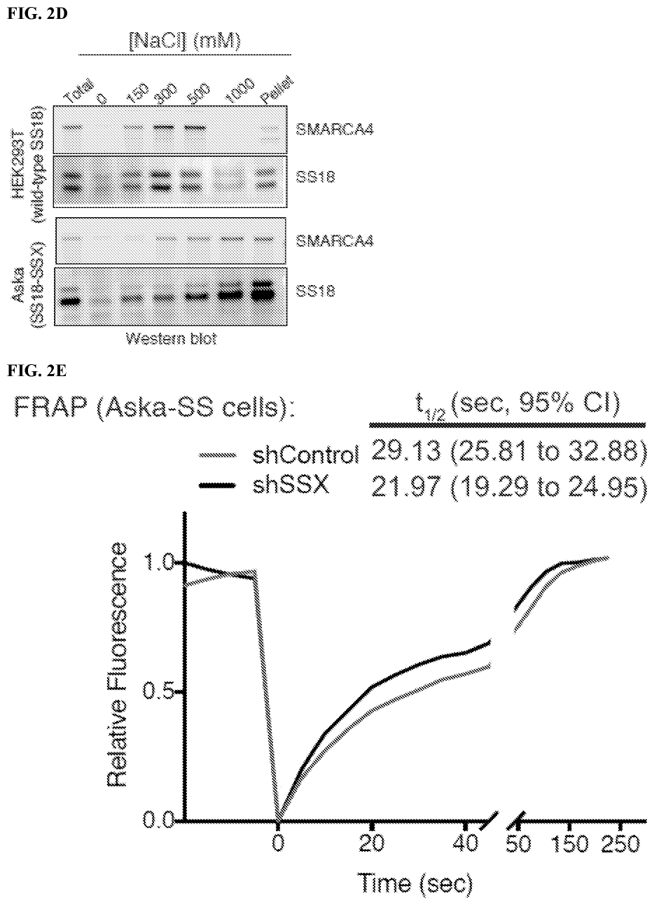

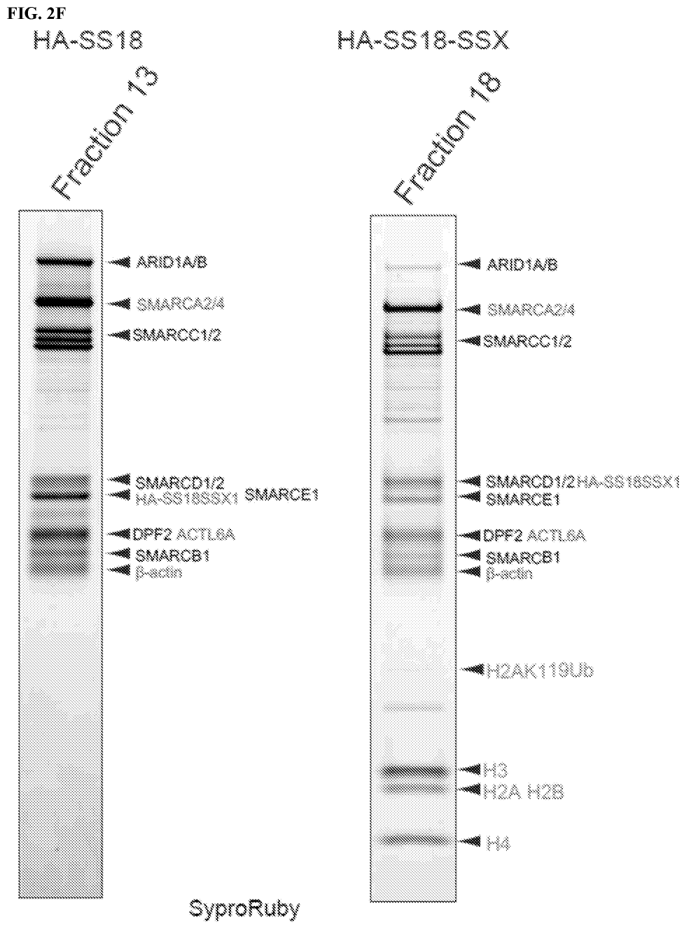

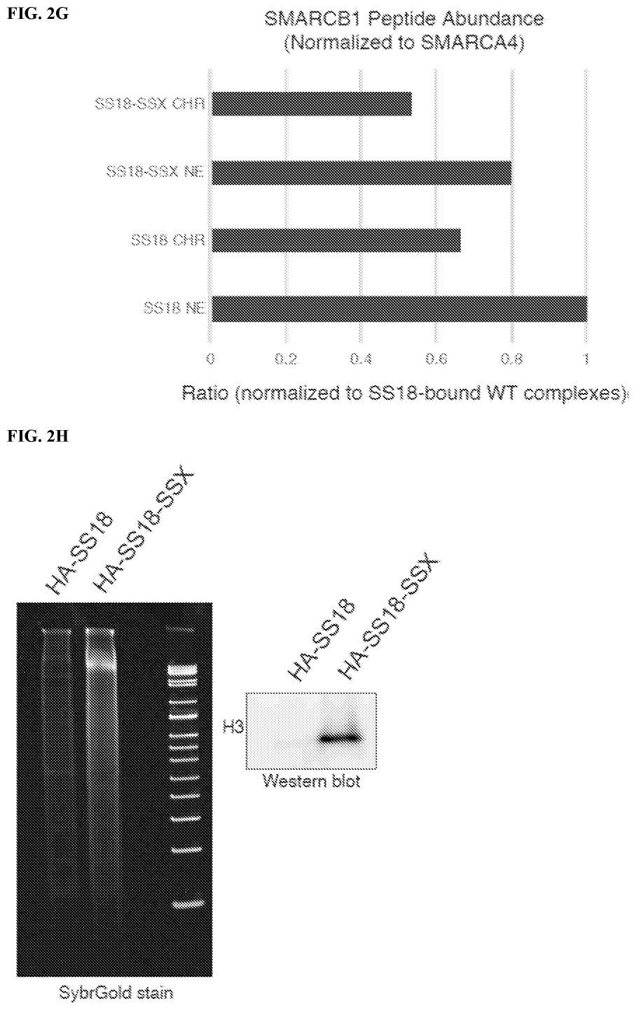

Interactions between chromatin-associated proteins and the histone landscape play major roles in dictating genome topology and gene expression. Cancer-specific fusion oncoproteins display unique chromatin localization patterns, yet often lack classical transcription factor-like DNA-binding domains, presenting challenges in identifying mechanisms governing their site-specific chromatin targeting and function. Recent studies indicate that SS18-SSX-bound BAF complexes have specialized biochemical and chromatin localization properties (McBride et al. (2018) Cancer Cell 33:1128-1141; Kadoch and Crabtree (2013) Cell 153:71-85). To explore the underlying molecular recognition mechanisms driving these associations and activities, HA-tagged versions of either wild-type (WT) SS18 or SS18-SSX were expressed in HEK-293T cells and BAF complex purifications were performed from soluble nuclear extract (NE) and nuclease-treated solubilized chromatin (CHR) (FIG. 1A). Strikingly, fusion oncoprotein SS18-SSX-bound BAF complexes preferentially eluted in the CHR material, in contrast to WT complexes, which eluted nearly completely in the soluble NE material, as expected from previous studies examining WT (and other loss-of-function mutant variants of) BAF complexes (Kadoch et al. (2013) Nat. Genetics 45:592-601; Mashtalir et al. (2018) Cell 175:1272-1288). Importantly, SS18-SSX-bound complexes captured near-stoichiometric amounts of core histone proteins H2A, H2B, H3, and H4 (FIG. 1A). The complexes were next subjected to mass-spectrometric (MS) analyses and selective co-enrichment of histone peptides with HA-SS18-SSX, but not with HA-WT SS18 was found in the chromatin-bound fractions (FIG. 1B, FIGS. 2A-2B). Notably, peptides corresponding to the H2A K119Ub mark were captured only in the purifications of SS18-SSX-bound complexes but not in SS18 WT complexes, in agreement with the visualization of this mark upon colloidal blue staining (FIG. 1A, Tables 5A-5E). In addition, it was found that SS18-SSX purifications most substantially enriched for ATPase subunits SMARCA4 and SMARCA2, BCL7A, and ACTL6A, consistent with the fact that SS18 is part of the ATPase module of mSWI/SNF complexes (Mashtalir et al. (2018) Cell 175:1272-1288), while core module components, particularly SMARCB1 were less enriched compared to WT SS18 purifications (FIG. 1B, FIGS. 2F and 2G, Tables 5A-5E). Binding to PRC1 components was not detected, as has been previously indicated (Banito et al. (2018) Cancer cell 33:527-541) (FIG. 2B).

TABLE 5A
HA-SS18SSX1_CHR_peptides

| Unique | Total | reference | Gene Symbol | MWT(kDa) | AVG |
|---|---|---|---|---|---|
| 55 | 584 | sp\|P51532\|SMCA4_HUMAN | SMARCA4 | 184.53 | 3.196 |
| 84 | 490 | sp\|P51531\|SMCA2_HUMAN | SMARCA2 | 181.17 | 2.9458 |
| 12 | 366 | sp\|P33778\|H2B1B_HUMAN | HIST1H2BB | 13.94 | 2.4497 |
| 9 | 214 | sp\|Q96KK5\|H2A1H_HUMAN | HIST1H2AH | 13.9 | 2.5116 |
| 69 | 167 | sp\|O14497\|ARI1A_HUMAN | ARID1A | 241.89 | 3.2645 |
| 51 | 165 | sp\|Q8TAQ2\|SMRC2_HUMAN | SMARCC2 | 132.8 | 2.9885 |
| 29 | 160 | sp\|O96019\|ACL6A_HUMAN | ACTL6A | 47.43 | 3.1707 |
| 39 | 133 | sp\|Q92922\|SMRC1_HUMAN | SMARCC1 | 122.79 | 3.0345 |
| 64 | 125 | sp\|Q8NFD5\|ARI1B_HUMAN | ARID1B | 235.97 | 3.0308 |
| 12 | 125 | sp\|P62805\|H4_HUMAN | HIST1H4A | 11.36 | 2.7795 |
| 12 | 119 | sp\|P62736\|ACTA_HUMAN | ACTA2 | 41.98 | 2.5744 |
| 34 | 78 | sp\|Q9NYF8\|BCLF1_HUMAN | BCLAF1 | 106.06 | 3.1403 |
| 57 | 73 | sp\|Q14839\|CHD4_HUMAN | CHD4 | 217.87 | 3.3514 |
| 9 | 73 | sp\|P63261\|ACTG_HUMAN | ACTG1 | 41.77 | 3.283 |
| 12 | 70 | sp\|Q4VC05\|BCL7A_HUMAN | BCL7A | 22.8 | 3.4877 |
| 62 | 68 | sp\|O75691\|UTP20_HUMAN | UTP20 | 318.18 | 3.3196 |
| 25 | 67 | sp\|Q9H307\|PININ_HUMAN | PNN | 81.56 | 3.0645 |
| 27 | 66 | sp\|Q6STE5\|SMRD3_HUMAN | SMARCD3 | 54.98 | 3.1596 |
| 25 | 61 | sp\|Q969G3\|SMCE1_HUMAN | SMARCE1 | 46.62 | 3.3347 |
| 25 | 61 | sp\|Q96GM5\|SMRD1_HUMAN | SMARCD1 | 58.2 | 3.3292 |
| 4 | 60 | sp\|Q16384\|SSX1_HUMAN | SSX1 | 21.92 | 3.3725 |
| 20 | 57 | sp\|Q12824\|SNF5_HUMAN | SMARCB1 | 44.11 | 3.1529 |
| 21 | 55 | sp\|Q00839\|HNRPU_HUMAN | HNRNPU | 90.53 | 3.0794 |
| 27 | 54 | sp\|Q9UKV3\|ACINU_HUMAN | ACIN1 | 151.77 | 2.983 |
| 33 | 51 | sp\|Q9H0A0\|NAT10_HUMAN | NAT10 | 115.66 | 3.4608 |
| 17 | 49 | sp\|P22087\|FBRL_HUMAN | FBL | 33.76 | 2.9946 |
| 23 | 47 | sp\|Q92925\|SMRD2_HUMAN | SMARCD2 | 58.88 | 3.3416 |
| 42 | 45 | sp\|Q8WYP5\|ELYS_HUMAN | AHCTF1 | 252.34 | 3.4462 |
| 22 | 45 | sp\|Q9Y2W1\|TR150_HUMAN | THRAP3 | 108.6 | 3.1033 |
| 42 | 44 | sp\|Q14980\|NUMA1_HUMAN | NUMA1 | 238.12 | 3.8461 |
| 42 | 44 | sp\|O75643\|U520_HUMAN | SNRNP200 | 244.35 | 3.2527 |
| 38 | 42 | sp\|Q6P2Q9\|PRP8_HUMAN | PRPF8 | 273.43 | 3.3056 |
| 38 | 42 | sp\|P11388\|TOP2A_HUMAN | TOP2A | 174.28 | 3.2238 |
| 31 | 42 | sp\|O00567\|NOP56_HUMAN | NOP56 | 66.01 | 3.3301 |
| 24 | 38 | sp\|P52272\|HNRPM_HUMAN | HNRNPM | 77.46 | 3.1869 |
| 20 | 38 | sp\|P08670\|VIME_HUMAN | VIM | 53.62 | 3.2563 |
| 31 | 37 | sp\|Q08211\|DHX9_HUMAN | DHX9 | 140.87 | 3.568 |
| 13 | 36 | sp\|Q14978\|NOLC1_HUMAN | NOLC1 | 73.56 | 2.6167 |
| 21 | 33 | sp\|P20700\|LMNB1_HUMAN | LMNB1 | 66.37 | 3.162 |
| 24 | 32 | sp\|O60264\|SMCA5_HUMAN | SMARCA5 | 121.83 | 3.1653 |
| 5 | 32 | sp\|P62987\|RL40_HUMAN | UBA52 | 14.72 | 2.7242 |
| 31 | 31 | sp\|Q9H583\|HEAT1_HUMAN | HEATR1 | 242.22 | 3.443 |
| 4 | 31 | sp\|Q71DI3\|H32_HUMAN | HIST2H3A | 15.38 | 1.8646 |
| 11 | 29 | sp\|Q8WUZ0\|BCL7C_HUMAN | BCL7C | 23.45 | 3.4264 |
| 9 | 29 | sp\|P45973\|CBX5_HUMAN | CBX5 | 22.21 | 2.805 |
| 27 | 28 | sp\|O75533\|SF3B1_HUMAN | SF3B1 | 145.74 | 3.3041 |
| 26 | 28 | sp\|Q9UIG0\|BAZ1B_HUMAN | BAZ1B | 170.8 | 3.4813 |
| 26 | 28 | sp\|Q9Y5B9\|SP16H_HUMAN | SUPT16H | 119.84 | 3.4761 |
| 20 | 27 | sp\|P17480\|UBF1_HUMAN | UBTF | 89.35 | 3.043 |
| 26 | 26 | sp\|Q14690\|RRP5_HUMAN | PDCD11 | 208.57 | 3.5217 |
| 24 | 26 | sp\|Q96T58\|MINT_HUMAN | SPEN | 402 | 3.2629 |
| 19 | 26 | sp\|Q9Y2X3\|NOP58_HUMAN | NOP58 | 59.54 | 4.0996 |
| 19 | 26 | sp\|Q08945\|SSRP1_HUMAN | SSRP1 | 81.02 | 3.2852 |
| 23 | 25 | sp\|Q9NZM4\|BICRA_HUMAN | BICRA | 158.39 | 3.5192 |
| 11 | 25 | sp\|Q07955\|SRSF1_HUMAN | SRSF1 | 27.73 | 2.7723 |
| 23 | 24 | sp\|Q6PL18\|ATAD2_HUMAN | ATAD2 | 158.46 | 3.5607 |
| 20 | 23 | sp\|P46100\|ATRX_HUMAN | ATRX | 282.41 | 3.3981 |
| 17 | 23 | sp\|P25440\|BRD2_HUMAN | BRD2 | 88.01 | 3.4653 |
| 8 | 23 | sp\|Q15287\|RNPS1_HUMAN | RNPS1 | 34.19 | 2.513 |
| 20 | 22 | sp\|Q14676\|MDC1_HUMAN | MDC1 | 226.53 | 3.4061 |
| 19 | 20 | sp\|Q14692\|BMS1_HUMAN | BMS1 | 145.72 | 3.6681 |
| 19 | 20 | sp\|P46013\|KI67_HUMAN | MKI67 | 358.47 | 3.0296 |
| 18 | 20 | sp\|Q9H8M2\|BRD9_HUMAN | BRD9 | 66.96 | 3.7468 |
| 18 | 20 | sp\|Q96T23\|RSF1_HUMAN | RSF1 | 163.72 | 3.644 |
| 18 | 20 | sp\|Q15393\|SF3B3_HUMAN | SF3B3 | 135.49 | 3.5038 |
| 18 | 20 | sp\|Q02880\|TOP2B_HUMAN | TOP2B | 183.15 | 3.4782 |
| 14 | 20 | sp\|Q92785\|REQU_HUMAN | DPF2 | 44.13 | 4.0099 |
| 14 | 20 | sp\|O75367\|H2AY_HUMAN | H2AFY | 39.59 | 3.3191 |
| 13 | 20 | sp\|P43243\|MATR3_HUMAN | MATR3 | 94.56 | 3.2566 |
| 12 | 20 | sp\|Q1KMD3\|HNRL2_HUMAN | HNRNPUL2 | 85.05 | 3.3094 |
| 3 | 20 | sp\|Q01130\|SRSF2_HUMAN | SRSF2 | 25.46 | 4.6781 |
| 18 | 19 | sp\|Q6KC79\|NIPBL_HUMAN | NIPBL | 315.85 | 3.4965 |
| 18 | 19 | sp\|O14646\|CHD1_HUMAN | CHD1 | 196.57 | 3.3084 |
| 17 | 19 | sp\|Q9BQG0\|MBB1A_HUMAN | MYBBP1A | 148.76 | 3.1516 |
| 15 | 19 | sp\|P46087\|NOP2_HUMAN | NOP2 | 89.25 | 3.5025 |
| 17 | 18 | sp\|Q8WWQ0\|PHIP_HUMAN | PHIP | 206.56 | 3.1097 |
| 15 | 18 | sp\|Q12905\|ILF2_HUMAN | ILF2 | 43.04 | 3.9059 |
| 15 | 18 | sp\|Q8IXT5\|RB12B_HUMAN | RBM12B | 118.03 | 2.9192 |
| 2 | 18 | sp\|Q9BRL6\|SRSF8_HUMAN | SRSF8 | 32.27 | 1.8838 |
| 17 | 17 | sp\|P78527\|PRKDC_HUMAN | PRKDC | 468.79 | 3.4649 |
| 17 | 17 | sp\|Q15029\|U5S1_HUMAN | EFTUD2 | 109.37 | 3.3215 |
| 15 | 17 | sp\|Q13435\|SF3B2_HUMAN | SF3B2 | 100.16 | 3.6324 |
| 9 | 17 | sp\|P50402\|EMD_HUMAN | EMD | 28.98 | 2.4119 |
| 7 | 17 | sp\|Q5BKZ1\|ZN326_HUMAN | ZNF326 | 65.61 | 2.8312 |
| 15 | 16 | sp\|Q14683\|SMC1A_HUMAN | SMC1A | 143.14 | 3.0529 |
| 14 | 16 | sp\|Q86U86\|PB1_HUMAN | PBRM1 | 192.83 | 3.4056 |
| 13 | 16 | sp\|Q12906\|ILF3_HUMAN | ILF3 | 95.28 | 3.2276 |
| 11 | 16 | sp\|Q9BVJ6\|UT14A_HUMAN | UTP14A | 87.92 | 3.5193 |
| 14 | 15 | sp\|Q9H2P0\|ADNP_HUMAN | ADNP | 123.49 | 3.5487 |
| 14 | 15 | sp\|Q7Z3K3\|POGZ_HUMAN | POGZ | 155.24 | 3.486 |
| 14 | 15 | sp\|Q9Y3T9\|NOC2L_HUMAN | NOC2L | 84.87 | 3.4078 |
| 13 | 15 | sp\|Q9P0M6\|H2AW_HUMAN | H2AFY2 | 40.03 | 3.4783 |
| 12 | 15 | sp\|O76021\|RL1D1_HUMAN | RSL1D1 | 54.94 | 3.0238 |
| 14 | 14 | sp\|Q03164\|KMT2A_HUMAN | KMT2A | 431.5 | 4.0265 |
| 14 | 14 | sp\|Q9UIF9\|BAZ2A_HUMAN | BAZ2A | 211.07 | 3.4659 |
| 13 | 14 | sp\|Q13620\|CUL4B_HUMAN | CUL4B | 103.92 | 3.6501 |
| 12 | 14 | sp\|O60306\|AQR_HUMAN | AQR | 171.19 | 3.1564 |
| 10 | 14 | sp\|Q6AI39\|BICRL_HUMAN | BICRAL | 115.01 | 3.6798 |
| 10 | 14 | sp\|Q9NY61\|AATF_HUMAN | AATF | 63.09 | 3.3347 |
| 3 | 14 | tr\|B9EGQ8\|B9EGQ8_HUMAN | SMARCA4 | 189.33 | 3.1403 |
| 2 | 14 | sp\|Q15532\|SSXT_HUMAN | SS18 | 45.9 | 2.6438 |
| 13 | 13 | sp\|Q9UK61\|TASOR_HUMAN | FAM208A | 188.91 | 3.4606 |
| 13 | 13 | sp\|Q9NTI5\|PDS5B_HUMAN | PDS5B | 164.56 | 2.9322 |
| 12 | 13 | sp\|P23396\|RS3_HUMAN | RPS3 | 26.67 | 2.7633 |
| 11 | 13 | sp\|Q49A26\|GLYR1_HUMAN | GLYR1 | 60.52 | 3.509 |
| 9 | 13 | sp\|P55795\|HNRH2_HUMAN | HNRNPH2 | 49.23 | 3.2887 |
| 9 | 13 | sp\|Q86VM9\|ZCH18_HUMAN | ZC3H18 | 106.32 | 2.9032 |
| 9 | 13 | sp\|Q13247\|SRSF6_HUMAN | SRSF6 | 39.56 | 2.4674 |
| 3 | 13 | sp\|Q71UI9\|H2AV_HUMAN | H2AFV | 13.5 | 2.7796 |
| 12 | 12 | sp\|P24928\|RPB1_HUMAN | POLR2A | 217.04 | 3.3463 |
| 12 | 12 | sp\|O43143\|DHX15_HUMAN | DHX15 | 90.88 | 3.2449 |
| 12 | 12 | sp\|Q96GQ7\|DDX27_HUMAN | DDX27 | 89.78 | 3.0281 |
| 6 | 12 | sp\|P31943\|HNRH1_HUMAN | HNRNPH1 | 49.2 | 2.9519 |
| 11 | 11 | sp\|O60216\|RAD21_HUMAN | RAD21 | 71.64 | 3.9259 |
| 11 | 11 | sp\|P09874\|PARP1_HUMAN | PARP1 | 113.01 | 3.4647 |
| 11 | 11 | sp\|Q12788\|TBL3_HUMAN | TBL3 | 88.98 | 3.4469 |
| 11 | 11 | sp\|P33993\|MCM7_HUMAN | MCM7 | 81.26 | 3.3527 |
| 11 | 11 | sp\|Q9NYH9\|UTP6_HUMAN | UTP6 | 70.15 | 3.3108 |
| 11 | 11 | sp\|Q8TDD1\|DDX54_HUMAN | DDX54 | 98.53 | 3.2741 |
| 11 | 11 | sp\|Q15397\|PUM3_HUMAN | PUM3 | 73.54 | 3.2129 |
| 11 | 11 | sp\|P28370\|SMCA1_HUMAN | SMARCA1 | 122.53 | 2.7975 |
| 11 | 11 | sp\|Q13619\|CUL4A_HUMAN | CUL4A | 87.62 | 2.6132 |
| 10 | 11 | sp\|Q12873\|CHD3_HUMAN | CHD3 | 226.45 | 3.7322 |
| 10 | 11 | sp\|Q8IZL8\|PELP1_HUMAN | PELP1 | 119.62 | 3.6196 |
| 10 | 11 | sp\|P18583\|SON_HUMAN | SON | 263.66 | 3.395 |
| 10 | 11 | sp\|Q99549\|MPP8_HUMAN | MPHOSPH8 | 97.12 | 3.3621 |
| 10 | 11 | sp\|Q9UKM9\|RALY_HUMAN | RALY | 32.44 | 3.1183 |
| 9 | 11 | sp\|O00541\|PESC_HUMAN | PES1 | 67.96 | 2.4036 |
| 7 | 11 | sp\|Q13243\|SRSF5_HUMAN | SRSF5 | 31.25 | 3.5127 |
| 6 | 11 | sp\|Q16629\|SRSF7_HUMAN | SRSF7 | 27.35 | 2.6912 |
| 4 | 11 | sp\|P16403\|H12_HUMAN | HIST1H1C | 21.35 | 2.8282 |
| 2 | 11 | sp\|Q02539\|H11_HUMAN | HIST1H1A | 21.83 | 2.9123 |
| 2 | 11 | sp\|Q93079\|H2B1H_HUMAN | HIST1H2BH | 13.88 | 2.2042 |
| 10 | 10 | sp\|Q99459\|CDC5L_HUMAN | CDC5L | 92.19 | 3.4411 |
| 10 | 10 | sp\|P38919\|IF4A3_HUMAN | EIF4A3 | 46.84 | 3.4146 |
| 10 | 10 | sp\|Q8N7H5\|PAF1_HUMAN | PAF1 | 59.94 | 3.3744 |
| 10 | 10 | sp\|Q6PD62\|CTR9_HUMAN | CTR9 | 133.42 | 3.368 |
| 10 | 10 | sp\|Q92794\|KAT6A_HUMAN | KAT6A | 224.89 | 3.2929 |
| 10 | 10 | sp\|Q8IWA0\|WDR75_HUMAN | WDR75 | 94.44 | 3.1652 |
| 10 | 10 | sp\|Q9ULI0\|ATD2B_HUMAN | ATAD2B | 164.81 | 3.1043 |
| 10 | 10 | sp\|Q03188\|CENPC_HUMAN | CENPC | 106.77 | 3.0478 |
| 10 | 10 | sp\|Q9NVP1\|DDX18_HUMAN | DDX18 | 75.36 | 2.7626 |
| 10 | 10 | sp\|Q9UQ35\|SRRM2_HUMAN | SRRM2 | 299.44 | 2.7014 |
| 9 | 10 | sp\|Q9Y4W2\|LAS1L_HUMAN | LAS1L | 83.01 | 3.8251 |
| 9 | 10 | sp\|Q9Y2R4\|DDX52_HUMAN | DDX52 | 67.46 | 3.626 |
| 9 | 10 | sp\|Q14202\|ZMYM3_HUMAN | ZMYM3 | 152.28 | 3.4516 |
| 9 | 10 | sp\|Q9NR30\|DDX21_HUMAN | DDX21 | 87.29 | 3.4294 |
| 9 | 10 | sp\|P62701\|RS4X_HUMAN | RPS4X | 29.58 | 3.093 |
| 9 | 10 | sp\|Q13601\|KRR1_HUMAN | KRR1 | 43.64 | 3.0329 |
| 9 | 10 | sp\|Q92841\|DDX17_HUMAN | DDX17 | 80.22 | 2.8111 |
| 8 | 10 | sp\|Q8NC56\|LEMD2_HUMAN | LEMD2 | 56.94 | 3.249 |
| 8 | 10 | sp\|Q7Z7K6\|CENPV_HUMAN | CENPV | 29.93 | 3.2113 |
| 9 | 9 | sp\|P57740\|NU107_HUMAN | NUP107 | 106.31 | 3.6499 |
| 9 | 9 | sp\|Q03701\|CEBPZ_HUMAN | CEBPZ | 120.9 | 3.5391 |
| 9 | 9 | sp\|O60281\|ZN292_HUMAN | ZNF292 | 304.62 | 3.2897 |
| 9 | 9 | sp\|Q13330\|MTA1_HUMAN | MTA1 | 80.74 | 3.2111 |
| 9 | 9 | sp\|O43390\|HNRPR_HUMAN | HNRNPR | 70.9 | 3.1736 |
| 9 | 9 | sp\|P49750\|YLPM1_HUMAN | YLPM1 | 219.85 | 3.1531 |
| 9 | 9 | sp\|Q9BSC4\|NOL10_HUMAN | NOL10 | 80.25 | 3.0247 |
| 9 | 9 | sp\|Q8N1F7\|NUP93_HUMAN | NUP93 | 93.43 | 2.9512 |
| 9 | 9 | sp\|Q7Z5J4\|RAI1_HUMAN | RAI1 | 203.23 | 2.939 |
| 9 | 9 | sp\|Q92621\|NU205_HUMAN | NUP205 | 227.78 | 2.9163 |
| 8 | 9 | sp\|Q9Y5J1\|UTP18_HUMAN | UTP18 | 61.96 | 3.6186 |
| 8 | 9 | sp\|Q9Y5B6\|PAXB1_HUMAN | PAXBP1 | 104.74 | 3.4559 |
| 8 | 9 | sp\|P12236\|ADT3_HUMAN | SLC25A6 | 32.85 | 2.8579 |
| 8 | 9 | sp\|P22626\|ROA2_HUMAN | HNRNPA2B1 | 37.41 | 2.8007 |
| 8 | 9 | sp\|P02545\|LMNA_HUMAN | LMNA | 74.09 | 2.7943 |
| 4 | 9 | sp\|P07305\|H10_HUMAN | H1F0 | 20.85 | 3.1846 |
| 8 | 8 | sp\|Q9UH99\|SUN2_HUMAN | SUN2 | 80.26 | 4.2804 |
| 8 | 8 | sp\|Q9NXF1\|TEX10_HUMAN | TEX10 | 105.61 | 3.9844 |
| 8 | 8 | sp\|O15213\|WDR46_HUMAN | WDR46 | 68.03 | 3.9455 |
| 8 | 8 | sp\|O00159\|MYO1C_HUMAN | MYO1C | 121.61 | 3.5705 |
| 8 | 8 | sp\|Q8N3U4\|STAG2_HUMAN | STAG2 | 141.24 | 3.4872 |
| 8 | 8 | sp\|Q9BVP2\|GNL3_HUMAN | GNL3 | 61.95 | 3.4513 |
| 8 | 8 | sp\|Q5QJE6\|TDIF2_HUMAN | DNTTIP2 | 84.42 | 3.3455 |
| 8 | 8 | sp\|Q16531\|DDB1_HUMAN | DDB1 | 126.89 | 3.3262 |
| 8 | 8 | sp\|Q6ZRS2\|SRCAP_HUMAN | SRCAP | 343.34 | 3.1877 |
| 7 | 8 | sp\|Q9UMS4\|PRP19_HUMAN | PRPF19 | 55.15 | 3.6093 |
| 7 | 8 | sp\|Q9H6F5\|CCD86_HUMAN | CCDC86 | 40.21 | 3.5931 |
| 7 | 8 | sp\|Q9BZE4\|NOG1_HUMAN | GTPBP4 | 73.92 | 3.1582 |
| 7 | 8 | sp\|O60832\|DKC1_HUMAN | DKC1 | 57.64 | 3.1341 |
| 7 | 8 | sp\|P39019\|RS19_HUMAN | RPS19 | 16.05 | 2.5209 |
| 5 | 8 | sp\|Q9BQE9\|BCL7B_HUMAN | BCL7B | 22.18 | 2.9657 |
| 4 | 8 | sp\|Q13595\|TRA2A_HUMAN | TRA2A | 32.67 | 2.8133 |
| 7 | 7 | sp\|O00566\|MPP10_HUMAN | MPHOSPH10 | 78.82 | 4.0635 |
| 7 | 7 | sp\|Q8IY81\|SPB1_HUMAN | FTSJ3 | 96.5 | 3.8802 |
| 7 | 7 | sp\|Q8WXH0\|SYNE2_HUMAN | SYNE2 | 795.94 | 3.7401 |
| 7 | 7 | sp\|O95251\|KAT7_HUMAN | KAT7 | 70.6 | 3.5459 |
| 7 | 7 | sp\|P42285\|SK2L2_HUMAN | SKIV2L2 | 117.73 | 3.4754 |
| 7 | 7 | sp\|P42167\|LAP2B_HUMAN | TMPO | 50.64 | 3.3395 |
| 7 | 7 | sp\|O14647\|CHD2_HUMAN | CHD2 | 211.21 | 3.2097 |
| 7 | 7 | sp\|Q9NRL2\|BAZ1A_HUMAN | BAZ1A | 178.59 | 3.2083 |
| 7 | 7 | sp\|Q13129\|RLF_HUMAN | RLF | 217.81 | 3.1787 |
| 7 | 7 | sp\|Q13111\|CAF1A_HUMAN | CHAF1A | 106.86 | 3.1007 |
| 7 | 7 | sp\|P68371\|TBB4B_HUMAN | TUBB4B | 49.8 | 2.9823 |
| 7 | 7 | sp\|P78316\|NOP14_HUMAN | NOP14 | 97.61 | 2.902 |
| 7 | 7 | sp\|O15042\|SR140_HUMAN | U2SURP | 118.22 | 2.8143 |
| 7 | 7 | sp\|Q5SSJ5\|HP1B3_HUMAN | HP1BP3 | 61.17 | 2.7313 |
| 7 | 7 | sp\|Q9NQS7\|INCE_HUMAN | INCENP | 105.36 | 2.6037 |
| 6 | 7 | sp\|Q13185\|CBX3_HUMAN | CBX3 | 20.8 | 3.6562 |
| 6 | 7 | sp\|P21796\|VDAC1_HUMAN | VDAC1 | 30.75 | 3.204 |
| 6 | 7 | sp\|P36578\|RL4_HUMAN | RPL4 | 47.67 | 2.6157 |
| 5 | 7 | sp\|Q92784\|DPF3_HUMAN | DPF3 | 43.06 | 3.5672 |
| 5 | 7 | sp\|O00422\|SAP18_HUMAN | SAP18 | 17.55 | 3.3178 |
| 5 | 7 | sp\|Q9H9B1\|EHMT1_HUMAN | EHMT1 | 141.38 | 3.3147 |
| 5 | 7 | sp\|P62995\|TRA2B_HUMAN | TRA2B | 33.65 | 3.2146 |
| 5 | 7 | IGH1M_MOUSE | Ighg1 | 43.36 | 2.9952 |
| 5 | 7 | sp\|Q8TDI0\|CHD5_HUMAN | CHD5 | 222.91 | 2.86 |
| 4 | 7 | sp\|P07910\|HNRPC_HUMAN | HNRNPC | 33.65 | 3.3974 |
| 6 | 6 | sp\|Q15061\|WDR43_HUMAN | WDR43 | 74.84 | 4.277 |
| 6 | 6 | sp\|Q9NQZ2\|SAS10_HUMAN | UTP3 | 54.53 | 4.2012 |
| 6 | 6 | sp\|Q15269\|PWP2_HUMAN | PWP2 | 102.39 | 4.0268 |
| 6 | 6 | sp\|P61978\|HNRPK_HUMAN | HNRNPK | 50.94 | 3.9336 |
| 6 | 6 | sp\|P19338\|NUCL_HUMAN | NCL | 76.57 | 3.9301 |
| 6 | 6 | sp\|Q7KZ85\|SPT6H_HUMAN | SUPT6H | 198.95 | 3.8795 |
| 6 | 6 | sp\|Q8WTT2\|NOC3L_HUMAN | NOC3L | 92.49 | 3.7112 |
| 6 | 6 | sp\|P08865\|RSSA_HUMAN | RPSA | 32.83 | 3.5636 |
| 6 | 6 | sp\|Q8TED0\|UTP15_HUMAN | UTP15 | 58.38 | 3.547 |
| 6 | 6 | sp\|Q96KQ7\|EHMT2_HUMAN | EHMT2 | 132.29 | 3.4908 |
| 6 | 6 | sp\|P51398\|RT29_HUMAN | DAP3 | 45.54 | 3.4773 |
| 6 | 6 | sp\|Q9BVI4\|NOC4L_HUMAN | NOC4L | 58.43 | 3.4602 |
| 6 | 6 | sp\|P07199\|CENPB_HUMAN | CENPB | 65.13 | 3.337 |
| 6 | 6 | sp\|Q9BQE3\|TBA1C_HUMAN | TUBA1C | 49.86 | 3.3028 |
| 6 | 6 | sp\|Q03252\|LMNB2_HUMAN | LMNB2 | 69.91 | 3.2923 |
| 6 | 6 | sp\|Q5VWN6\|F208B_HUMAN | FAM208B | 268.68 | 3.2658 |
| 6 | 6 | sp\|Q9HC52\|CBX8_HUMAN | CBX8 | 43.37 | 3.2482 |
| 6 | 6 | sp\|Q8IWI9\|MGAP_HUMAN | MGA | 331.63 | 3.2248 |
| 6 | 6 | sp\|Q8WXI9\|P66B_HUMAN | GATAD2B | 65.22 | 3.223 |
| 6 | 6 | sp\|Q13573\|SNW1_HUMAN | SNW1 | 61.46 | 3.1062 |
| 6 | 6 | sp\|Q9Y2P8\|RCL1_HUMAN | RCL1 | 40.82 | 3.0802 |
| 6 | 6 | sp\|B2RXH8\|HNRC2_HUMAN | HNRNPCL2 | 32.05 | 3.0374 |
| 6 | 6 | sp\|P52701\|MSH6_HUMAN | MSH6 | 152.69 | 2.9401 |
| 6 | 6 | sp\|Q16891\|MIC60_HUMAN | IMMT | 83.63 | 2.895 |
| 5 | 6 | sp\|P52597\|HNRPF_HUMAN | HNRNPF | 45.64 | 3.7652 |
| 5 | 6 | sp\|Q8NEJ9\|NGDN_HUMAN | NGDN | 35.87 | 3.6102 |
| 5 | 6 | sp\|Q8ND82\|Z280C_HUMAN | ZNF280C | 83.04 | 3.3478 |
| 5 | 6 | sp\|P38159\|RBMX_HUMAN | RBMX | 42.31 | 3.2052 |
| 5 | 6 | sp\|P15880\|RS2_HUMAN | RPS2 | 31.3 | 3.1733 |
| 5 | 6 | sp\|Q5VZL5\|ZMYM4_HUMAN | ZMYM4 | 172.68 | 2.9892 |
| 5 | 6 | sp\|P62753\|RS6_HUMAN | RPS6 | 28.66 | 2.8134 |
| 5 | 6 | sp\|P29375\|KDM5A_HUMAN | KDM5A | 191.97 | 2.7763 |
| 4 | 6 | sp\|Q5TAP6\|UT14C_HUMAN | UTP14C | 87.13 | 3.2114 |
| 3 | 6 | sp\|P16104\|H2AX_HUMAN | H2AFX | 15.14 | 3.72 |
| 5 | 5 | sp\|P83916\|CBX1_HUMAN | CBX1 | 21.4 | 4.365 |
| 5 | 5 | sp\|P52292\|IMA1_HUMAN | KPNA2 | 57.83 | 4.1255 |
| 5 | 5 | sp\|Q9HCS7\|SYF1_HUMAN | XAB2 | 99.95 | 4.0454 |
| 5 | 5 | sp\|Q9UNX4\|WDR3_HUMAN | WDR3 | 106.03 | 3.9842 |
| 5 | 5 | sp\|P55265\|DSRAD_HUMAN | ADAR | 135.98 | 3.8215 |
| 5 | 5 | sp\|Q9NU22\|MDN1_HUMAN | MDN1 | 632.42 | 3.7989 |
| 5 | 5 | sp\|Q12830\|BPTF_HUMAN | BPTF | 338.05 | 3.7277 |
| 5 | 5 | sp\|O75400\|PR40A_HUMAN | PRPF40A | 108.74 | 3.6993 |
| 5 | 5 | sp\|P49792\|RBP2_HUMAN | RANBP2 | 357.97 | 3.6351 |
| 5 | 5 | sp\|O94901\|SUN1_HUMAN | SUN1 | 90.01 | 3.6143 |
| 5 | 5 | tr\|A0A1P0AZG4\|A0A1P0AZG4_HUMAN | LCOR | 137.14 | 3.5821 |
| 5 | 5 | sp\|Q9UQE7\|SMC3_HUMAN | SMC3 | 141.45 | 3.5623 |
| 5 | 5 | sp\|Q14137\|BOP1_HUMAN | BOP1 | 83.58 | 3.5549 |
| 5 | 5 | sp\|P26368\|U2AF2_HUMAN | U2AF2 | 53.47 | 3.477 |
| 5 | 5 | sp\|P68104\|EF1A1_HUMAN | EEF1A1 | 50.11 | 3.3958 |
| 5 | 5 | sp\|Q8WVM7\|STAG1_HUMAN | STAG1 | 144.34 | 3.3576 |
| 5 | 5 | sp\|Q969X6\|UTP4_HUMAN | UTP4 | 76.84 | 3.3295 |
| 5 | 5 | sp\|Q12769\|NU160_HUMAN | NUP160 | 162.02 | 3.3228 |
| 5 | 5 | sp\|Q8WUM0\|NU133_HUMAN | NUP133 | 128.9 | 3.2892 |
| 5 | 5 | sp\|Q53HL2\|BOREA_HUMAN | CDCA8 | 31.3 | 3.2744 |
| 5 | 5 | sp\|Q14684\|RRP1B_HUMAN | RRP1B | 84.38 | 3.2428 |
| 5 | 5 | sp\|P49411\|EFTU_HUMAN | TUFM | 49.51 | 3.1661 |
| 5 | 5 | sp\|Q9H582\|ZN644_HUMAN | ZNF644 | 149.47 | 3.1597 |
| 5 | 5 | sp\|P62241\|RS8_HUMAN | RPS8 | 24.19 | 3.0978 |
| 5 | 5 | sp\|Q14103\|HNRPD_HUMAN | HNRNPD | 38.41 | 2.9787 |
| 5 | 5 | sp\|Q12931\|TRAP1_HUMAN | TRAP1 | 80.06 | 2.9565 |
| 5 | 5 | sp\|Q09028\|RBBP4_HUMAN | RBBP4 | 47.63 | 2.9205 |
| 5 | 5 | sp\|Q9BWN1\|PRR14_HUMAN | PRR14 | 64.29 | 2.8386 |
| 5 | 5 | sp\|Q96GD4\|AURKB_HUMAN | AURKB | 39.29 | 2.8373 |
| 5 | 5 | sp\|Q14739\|LBR_HUMAN | LBR | 70.66 | 2.8151 |
| 5 | 5 | sp\|Q13895\|BYST_HUMAN | BYSL | 49.57 | 2.5969 |
| 5 | 5 | sp\|Q9NY12\|GAR1_HUMAN | GAR1 | 22.33 | 2.4622 |
| 4 | 5 | sp\|Q96EU6\|RRP36_HUMAN | RRP36 | 29.8 | 3.6465 |
| 4 | 5 | sp\|P14866\|HNRPL_HUMAN | HNRNPL | 64.09 | 3.6299 |
| 4 | 5 | sp\|P14678\|RSMB_HUMAN | SNRPB | 24.59 | 3.375 |
| 4 | 5 | sp\|Q96KR1\|ZFR_HUMAN | ZFR | 116.94 | 2.776 |
| 4 | 5 | sp\|P63244\|RACK1_HUMAN | RACK1 | 35.05 | 2.595 |
| 4 | 5 | sp\|Q8WYB5\|KAT6B_HUMAN | KAT6B | 231.23 | 2.3396 |
| 4 | 5 | sp\|P62269\|RS18_HUMAN | RPS18 | 17.71 | 2.3307 |
| 3 | 5 | sp\|Q7Z4V5\|HDGR2_HUMAN | HDGFL2 | 74.27 | 3.5225 |
| 3 | 5 | sp\|Q00325\|MPCP_HUMAN | SLC25A3 | 40.07 | 3.4595 |
| 3 | 5 | sp\|Q8TF01\|PNISR_HUMAN | PNISR | 92.52 | 3.272 |
| 2 | 5 | sp\|P84103\|SRSF3_HUMAN | SRSF3 | 19.32 | 2.3291 |
| 2 | 5 | sp\|Q96PV6\|LENG8_HUMAN | LENG8 | 86.07 | 1.9933 |
| 4 | 4 | sp\|P32969\|RL9_HUMAN | RPL9 | 21.85 | 4.8881 |
| 4 | 4 | sp\|Q8N201\|INT1_HUMAN | INTS1 | 244.14 | 4.2509 |
| 4 | 4 | sp\|Q14498\|RBM39_HUMAN | RBM39 | 59.34 | 4.0875 |
| 4 | 4 | sp\|P12956\|XRCC6_HUMAN | XRCC6 | 69.8 | 3.9984 |
| 4 | 4 | sp\|Q13263\|TIF1B_HUMAN | TRIM28 | 88.49 | 3.7834 |
| 4 | 4 | sp\|Q96QD9\|UIF_HUMAN | FYTTD1 | 35.8 | 3.783 |
| 4 | 4 | sp\|Q9NPI1\|BRD7_HUMAN | BRD7 | 74.09 | 3.7737 |
| 4 | 4 | sp\|O60287\|NPA1P_HUMAN | URB1 | 254.23 | 3.7233 |
| 4 | 4 | sp\|P45880\|VDAC2_HUMAN | VDAC2 | 31.55 | 3.6583 |
| 4 | 4 | sp\|P62424\|RL7A_HUMAN | RPL7A | 29.98 | 3.6514 |
| 4 | 4 | sp\|Q9Y6K1\|DNM3A_HUMAN | DNMT3A | 101.79 | 3.5442 |
| 4 | 4 | sp\|Q9BYG3\|MK671_HUMAN | NIFK | 34.2 | 3.5075 |
| 4 | 4 | sp\|P35580\|MYH10_HUMAN | MYH10 | 228.86 | 3.5027 |
| 4 | 4 | sp\|P11142\|HSP7C_HUMAN | HSPA8 | 70.85 | 3.4819 |
| 4 | 4 | sp\|O60508\|PRP17_HUMAN | CDC40 | 65.48 | 3.4393 |
| 4 | 4 | sp\|O96028\|NSD2_HUMAN | NSD2 | 152.16 | 3.359 |
| 4 | 4 | sp\|O15164\|TIF1A_HUMAN | TRIM24 | 116.76 | 3.3318 |
| 4 | 4 | sp\|Q8IX01\|SUGP2_HUMAN | SUGP2 | 120.13 | 3.3282 |
| 4 | 4 | sp\|Q14966\|ZN638_HUMAN | ZNF638 | 220.49 | 3.3182 |
| 4 | 4 | sp\|Q9NVI7\|ATD3A_HUMAN | ATAD3A | 71.32 | 3.3045 |
| 4 | 4 | sp\|Q9Y6X3\|SCC4_HUMAN | MAU2 | 69.04 | 3.2931 |
| 4 | 4 | sp\|O94776\|MTA2_HUMAN | MTA2 | 74.98 | 3.2496 |
| 4 | 4 | sp\|Q69YN4\|VIR_HUMAN | KIAA1429 | 201.9 | 3.2469 |
| 4 | 4 | sp\|Q96L73\|NSD1_HUMAN | NSD1 | 296.46 | 3.2347 |
| 4 | 4 | sp\|Q9BTV4\|TMM43_HUMAN | TMEM43 | 44.85 | 3.22 |
| 4 | 4 | sp\|Q9H9B4\|SFXN1_HUMAN | SFXN1 | 35.6 | 3.2098 |
| 4 | 4 | sp\|O14979\|HNRDL_HUMAN | HNRNPDL | 46.41 | 3.2084 |
| 4 | 4 | sp\|Q5JTV8\|TOIP1_HUMAN | TOR1AIP1 | 66.21 | 3.1934 |
| 4 | 4 | sp\|Q08170\|SRSF4_HUMAN | SRSF4 | 56.65 | 3.1185 |
| 4 | 4 | sp\|Q7L2E3\|DHX30_HUMAN | DHX30 | 133.85 | 3.0745 |
| 4 | 4 | sp\|O94906\|PRP6_HUMAN | PRPF6 | 106.86 | 3.0464 |
| 4 | 4 | sp\|P18124\|RL7_HUMAN | RPL7 | 29.21 | 3.0421 |
| 4 | 4 | sp\|P33991\|MCM4_HUMAN | MCM4 | 96.5 | 3.0269 |
| 4 | 4 | sp\|Q8IY37\|DHX37_HUMAN | DHX37 | 129.46 | 3.0184 |
| 4 | 4 | sp\|Q53GS7\|GLE1_HUMAN | GLE1 | 79.79 | 3.016 |
| 4 | 4 | sp\|P17844\|DDX5_HUMAN | DDX5 | 69.1 | 2.9945 |
| 4 | 4 | sp\|Q9H0D6\|XRN2_HUMAN | XRN2 | 108.51 | 2.9774 |
| 4 | 4 | sp\|Q15059\|BRD3_HUMAN | BRD3 | 79.49 | 2.9604 |
| 4 | 4 | sp\|Q96PK6\|RBM14_HUMAN | RBM14 | 69.45 | 2.9572 |
| 4 | 4 | sp\|Q96G21\|IMP4_HUMAN | IMP4 | 33.74 | 2.9255 |
| 4 | 4 | sp\|P35658\|NU214_HUMAN | NUP214 | 213.49 | 2.9007 |
| 4 | 4 | sp\|Q86YP4\|P66A_HUMAN | GATAD2A | 68.02 | 2.8966 |
| 4 | 4 | sp\|Q07021\|C1QBP_HUMAN | C1QBP | 31.34 | 2.8754 |
| 4 | 4 | sp\|Q6DKI1\|RL7L_HUMAN | RPL7L1 | 28.64 | 2.8526 |
| 4 | 4 | sp\|O43795\|MYO1B_HUMAN | MYO1B | 131.9 | 2.8423 |
| 4 | 4 | sp\|Q86U38\|NOP9_HUMAN | NOP9 | 69.39 | 2.814 |
| 4 | 4 | sp\|Q96ME7\|ZN512_HUMAN | ZNF512 | 64.64 | 2.8133 |
| 4 | 4 | sp\|Q13242\|SRSF9_HUMAN | SRSF9 | 25.53 | 2.7693 |
| 4 | 4 | sp\|P62277\|RS13_HUMAN | RPS13 | 17.21 | 2.7392 |
| 4 | 4 | sp\|Q8N8A6\|DDX51_HUMAN | DDX51 | 72.41 | 2.7334 |
| 4 | 4 | sp\|P49756\|RBM25_HUMAN | RBM25 | 100.12 | 2.7244 |
| 4 | 4 | sp\|O75152\|ZC11A_HUMAN | ZC3H11A | 89.08 | 2.6256 |
| 4 | 4 | sp\|P62750\|RL23A_HUMAN | RPL23A | 17.68 | 2.5769 |
| 4 | 4 | sp\|Q96HS1\|PGAM5_HUMAN | PGAM5 | 31.98 | 2.4728 |
| 4 | 4 | sp\|P26373\|RL13_HUMAN | RPL13 | 24.25 | 2.3533 |
| 3 | 4 | sp\|O95218\|ZRAB2_HUMAN | ZRANB2 | 37.38 | 3.9985 |
| 3 | 4 | sp\|Q9NS69\|TOM22_HUMAN | TOMM22 | 15.51 | 3.9897 |
| 3 | 4 | sp\|Q9Y6A4\|CFA20_HUMAN | CFAP20 | 22.76 | 3.5149 |
| 3 | 4 | sp\|P78364\|PHC1_HUMAN | PHC1 | 105.47 | 3.4121 |
| 3 | 4 | sp\|Q96DI7\|SNR40_HUMAN | SNRNP40 | 39.29 | 3.1631 |
| 3 | 4 | sp\|Q9Y277\|VDAC3_HUMAN | VDAC3 | 30.64 | 2.7054 |
| 2 | 4 | sp\|P46783\|RS10_HUMAN | RPS10 | 18.89 | 2.7067 |
| 3 | 3 | sp\|P35453\|HXD13_HUMAN | HOXD13 | 36.08 | 4.379 |
| 3 | 3 | sp\|Q15459\|SF3A1_HUMAN | SF3A1 | 88.83 | 4.3736 |
| 3 | 3 | sp\|P56182\|RRP1_HUMAN | RRP1 | 52.81 | 4.3342 |
| 3 | 3 | sp\|P25705\|ATPA_HUMAN | ATP5A1 | 59.71 | 4.272 |
| 3 | 3 | sp\|P08708\|RS17_HUMAN | RPS17 | 15.54 | 4.2613 |
| 3 | 3 | sp\|P11021\|GRP78_HUMAN | HSPA5 | 72.29 | 4.2177 |
| 3 | 3 | sp\|Q92769\|HDAC2_HUMAN | HDAC2 | 55.33 | 4.101 |
| 3 | 3 | sp\|Q15424\|SAFB1_HUMAN | SAFB | 102.58 | 4.0441 |
| 3 | 3 | sp\|Q8WVC0\|LEO1_HUMAN | LEO1 | 75.36 | 3.8904 |
| 3 | 3 | sp\|O15523\|DDX3Y_HUMAN | DDX3Y | 73.11 | 3.8037 |
| 3 | 3 | sp\|P55201\|BRPF1_HUMAN | BRPF1 | 137.41 | 3.7974 |
| 3 | 3 | sp\|Q15050\|RRS1_HUMAN | RRS1 | 41.17 | 3.7946 |
| 3 | 3 | sp\|P62316\|SMD2_HUMAN | SNRPD2 | 13.52 | 3.6706 |
| 3 | 3 | sp\|P38432\|COIL_HUMAN | COIL | 62.57 | 3.6496 |
| 3 | 3 | sp\|Q9P035\|HACD3_HUMAN | HACD3 | 43.13 | 3.5854 |
| 3 | 3 | sp\|Q9H8H0\|NOL11_HUMAN | NOL11 | 81.07 | 3.5795 |
| 3 | 3 | sp\|Q9HAF1\|EAF6_HUMAN | MEAF6 | 21.62 | 3.578 |
| 3 | 3 | sp\|Q8IWX8\|CHERP_HUMAN | CHERP | 103.64 | 3.5545 |
| 3 | 3 | sp\|Q9BUJ2\|HNRL1_HUMAN | HNRNPUL1 | 95.68 | 3.5307 |
| 3 | 3 | sp\|Q9H8H2\|DDX31_HUMAN | DDX31 | 94.03 | 3.4706 |
| 3 | 3 | sp\|O94880\|PHF14_HUMAN | PHF14 | 99.99 | 3.4646 |
| 3 | 3 | sp\|Q5JTH9\|RRP12_HUMAN | RRP12 | 143.61 | 3.4367 |
| 3 | 3 | sp\|P56537\|IF6_HUMAN | EIF6 | 26.58 | 3.4285 |
| 3 | 3 | sp\|Q9UKJ3\|GPTC8_HUMAN | GPATCH8 | 164.1 | 3.3578 |
| 3 | 3 | sp\|Q07020\|RL18_HUMAN | RPL18 | 21.62 | 3.3553 |
| 3 | 3 | sp\|P06748\|NPM_HUMAN | NPM1 | 32.55 | 3.329 |
| 3 | 3 | sp\|Q3ZCQ8\|TIM50_HUMAN | TIMM50 | 39.62 | 3.3211 |
| 3 | 3 | sp\|Q14781\|CBX2_HUMAN | CBX2 | 56.05 | 3.3022 |
| 3 | 3 | sp\|Q9H7B2\|RPF2_HUMAN | RPF2 | 35.56 | 3.2995 |
| 3 | 3 | sp\|Q9UBB9\|TFP11_HUMAN | TFIP11 | 96.76 | 3.2993 |
| 3 | 3 | sp\|P61247\|RS3A_HUMAN | RPS3A | 29.93 | 3.2546 |
| 3 | 3 | sp\|P35251\|RFC1_HUMAN | RFC1 | 128.18 | 3.2541 |
| 3 | 3 | sp\|Q99848\|EBP2_HUMAN | EBNA1BP2 | 34.83 | 3.2005 |
| 3 | 3 | sp\|P34931\|HS71L_HUMAN | HSPA1L | 70.33 | 3.1654 |
| 3 | 3 | sp\|O95831\|AIFM1_HUMAN | AIFM1 | 66.86 | 3.1432 |
| 3 | 3 | tr\|F8VXC8\|F8VXC8_HUMAN | SMARCC2 | 136.1 | 3.1344 |
| 3 | 3 | sp\|Q5VT52\|RPRD2_HUMAN | RPRD2 | 155.92 | 3.1314 |
| 3 | 3 | sp\|Q13206\|DDX10_HUMAN | DDX10 | 100.83 | 3.0819 |
| 3 | 3 | sp\|P62081\|RS7_HUMAN | RPS7 | 22.11 | 3.0752 |
| 3 | 3 | sp\|Q01831\|XPC_HUMAN | XPC | 105.89 | 3.0057 |
| 3 | 3 | sp\|O15226\|NKRF_HUMAN | NKRF | 77.62 | 3.0054 |
| 3 | 3 | sp\|Q92522\|H1X_HUMAN | H1FX | 22.47 | 2.9991 |
| 3 | 3 | sp\|Q8WXF0\|SRS12_HUMAN | SRSF12 | 30.49 | 2.9422 |
| 3 | 3 | sp\|Q9NRZ9\|HELLS_HUMAN | HELLS | 97.01 | 2.8286 |
| 3 | 3 | sp\|P62851\|RS25_HUMAN | RPS25 | 13.73 | 2.8129 |
| 3 | 3 | sp\|P46781\|RS9_HUMAN | RPS9 | 22.58 | 2.7809 |
| 3 | 3 | sp\|Q13428\|TCOF_HUMAN | TCOF1 | 152.02 | 2.6949 |
| 3 | 3 | sp\|Q9H501\|ESF1_HUMAN | ESF1 | 98.73 | 2.6558 |
| 3 | 3 | sp\|Q9BZJ0\|CRNL1_HUMAN | CRNKL1 | 100.39 | 2.5946 |
| 3 | 3 | sp\|Q99496\|RING2_HUMAN | RNF2 | 37.63 | 2.5349 |
| 3 | 3 | sp\|Q8NI36\|WDR36_HUMAN | WDR36 | 105.26 | 2.4894 |
| 3 | 3 | sp\|Q9Y2R9\|RT07_HUMAN | MRPS7 | 28.12 | 2.4687 |
| 3 | 3 | sp\|P62249\|RS16_HUMAN | RPS16 | 16.44 | 2.4589 |
| 3 | 3 | sp\|P53999\|TCP4_HUMAN | SUB1 | 14.39 | 2.4304 |
| 3 | 3 | sp\|Q13123\|RED_HUMAN | IK | 65.56 | 2.3999 |
| 3 | 3 | sp\|P30876\|RPB2_HUMAN | POLR2B | 133.81 | 2.367 |
| 3 | 3 | sp\|P62906\|RL10A_HUMAN | RPL10A | 24.82 | 2.2409 |
| 3 | 3 | sp\|Q96H22\|CENPN_HUMAN | CENPN | 39.53 | 2.2018 |
| 3 | 3 | sp\|Q96T37\|RBM15_HUMAN | RBM15 | 107.12 | 2.0773 |
| 2 | 3 | tr\|B2R5W2\|B2R5W2_HUMAN | HNRNPC | 31.93 | 5.509 |
| 2 | 3 | sp\|P67809\|YBOX1_HUMAN | YBX1 | 35.9 | 4.8382 |
| 2 | 3 | sp\|Q9Y5S9\|RBM8A_HUMAN | RBM8A | 19.88 | 3.4783 |
| 2 | 3 | sp\|Q9UIS9\|MBD1_HUMAN | MBD1 | 66.56 | 3.2337 |
| 2 | 3 | sp\|Q66PJ3\|AR6P4_HUMAN | ARL6IP4 | 44.89 | 3.2302 |
| 2 | 3 | sp\|P17661\|DESM_HUMAN | DES | 53.5 | 2.986 |
| 2 | 3 | sp\|P82650\|RT22_HUMAN | MRPS22 | 41.25 | 2.8833 |
| 2 | 3 | sp\|P04406\|G3P_HUMAN | GAPDH | 36.03 | 2.5682 |
| 2 | 3 | sp\|P22090\|RS4Y1_HUMAN | RPS4Y1 | 29.44 | 2.4726 |
| 1 | 3 | tr\|Q6PJV4\|Q6PJV4_HUMAN | THRAP3 | 41.83 | 4.322 |
| 2 | 2 | sp\|P49711\|CTCF_HUMAN | CTCF | 82.73 | 5.3155 |
| 2 | 2 | sp\|Q68CP9\|ARID2_HUMAN | ARID2 | 197.27 | 4.6815 |
| 2 | 2 | sp\|Q9GZL7\|WDR12_HUMAN | WDR12 | 47.68 | 4.5006 |
| 2 | 2 | sp\|Q96IZ7\|RSRC1_HUMAN | RSRC1 | 38.65 | 4.4356 |
| 2 | 2 | sp\|Q9UGU0\|TCF20_HUMAN | TCF20 | 211.64 | 4.4306 |
| 2 | 2 | sp\|P0DMV9\|HS71B_HUMAN | HSPA1B | 70.01 | 4.4149 |
| 2 | 2 | sp\|Q14669\|TRIPC_HUMAN | TRIP12 | 220.3 | 4.3086 |
| 2 | 2 | sp\|Q5UIP0\|RIF1_HUMAN | RIF1 | 274.29 | 4.3075 |
| 2 | 2 | sp\|O00267\|SPT5H_HUMAN | SUPT5H | 120.92 | 4.2498 |
| 2 | 2 | sp\|P05388\|RLA0_HUMAN | RPLP0 | 34.25 | 4.2162 |
| 2 | 2 | sp\|Q9BYN8\|RT26_HUMAN | MRPS26 | 24.2 | 4.1072 |
| 2 | 2 | sp\|Q5SRE5\|NU188_HUMAN | NUP188 | 195.92 | 4.0991 |
| 2 | 2 | sp\|Q15637\|SF01_HUMAN | SF1 | 68.29 | 4.0782 |
| 2 | 2 | sp\|Q96SB8\|SMC6_HUMAN | SMC6 | 126.25 | 3.9457 |
| 2 | 2 | sp\|P20226\|TBP_HUMAN | TBP | 37.67 | 3.9353 |
| 2 | 2 | sp\|Q96MU7\|YTDC1_HUMAN | YTHDC1 | 84.65 | 3.9179 |
| 2 | 2 | sp\|Q92665\|RT31_HUMAN | MRPS31 | 45.29 | 3.8434 |
| 2 | 2 | sp\|Q92552\|RT27_HUMAN | MRPS27 | 47.58 | 3.8003 |
| 2 | 2 | sp\|Q9NV31\|IMP3_HUMAN | IMP3 | 21.84 | 3.7393 |
| 2 | 2 | sp\|P04844\|RPN2_HUMAN | RPN2 | 69.24 | 3.7317 |
| 2 | 2 | sp\|Q32P51\|RA1L2_HUMAN | HNRNPA1L2 | 34.2 | 3.6858 |
| 2 | 2 | sp\|P05141\|ADT2_HUMAN | SLC25A5 | 32.83 | 3.6825 |
| 2 | 2 | sp\|Q8N1GO\|ZN687_HUMAN | ZNF687 | 129.45 | 3.6672 |
| 2 | 2 | sp\|Q96EY7\|PTCD3_HUMAN | PTCD3 | 78.5 | 3.6382 |
| 2 | 2 | sp\|O75494\|SRS10_HUMAN | SRSF10 | 31.28 | 3.634 |
| 2 | 2 | sp\|O75531\|BAF_HUMAN | BANF1 | 10.05 | 3.6281 |
| 2 | 2 | sp\|Q13427\|PPIG_HUMAN | PPIG | 88.56 | 3.6088 |
| 2 | 2 | tr\|A0A0A0MQS2\|A0A0A0MQS2_HUMAN | CLASRP | 77.12 | 3.5848 |
| 2 | 2 | sp\|Q9UNQ2\|DIM1_HUMAN | DIMT1 | 35.21 | 3.5846 |
| 2 | 2 | sp\|P39023\|RL3_HUMAN | RPL3 | 46.08 | 3.569 |
| 2 | 2 | sp\|P41219\|PERI_HUMAN | PRPH | 53.62 | 3.5672 |
| 2 | 2 | sp\|Q8WYH8\|ING5_HUMAN | ING5 | 27.73 | 3.5567 |
| 2 | 2 | sp\|Q15022\|SUZ12_HUMAN | SUZ12 | 83 | 3.543 |
| 2 | 2 | sp\|P26599\|PTBP1_HUMAN | PTBP1 | 57.19 | 3.5251 |
| 2 | 2 | sp\|P07196\|NFL_HUMAN | NEFL | 61.48 | 3.5125 |
| 2 | 2 | sp\|Q69YH5\|CDCA2_HUMAN | CDCA2 | 112.61 | 3.5117 |
| 2 | 2 | sp\|O95478\|NSA2_HUMAN | NSA2 | 30.05 | 3.5067 |
| 2 | 2 | sp\|O75151\|PHF2_HUMAN | PHF2 | 120.7 | 3.5008 |
| 2 | 2 | sp\|P49736\|MCM2_HUMAN | MCM2 | 101.83 | 3.4994 |
| 2 | 2 | sp\|P35637\|FUS_HUMAN | FUS | 53.39 | 3.4823 |
| 2 | 2 | sp\|P07437\|TBB5_HUMAN | TUBB | 49.64 | 3.4431 |
| 2 | 2 | sp\|Q9Y3A2\|UTP11_HUMAN | UTP11 | 30.43 | 3.4209 |
| 2 | 2 | sp\|Q8TDN6\|BRX1_HUMAN | BRIX1 | 41.37 | 3.3781 |
| 2 | 2 | sp\|Q9HCG8\|CWC22_HUMAN | CWC22 | 105.4 | 3.3528 |
| 2 | 2 | sp\|Q9NQ39\|RS10L_HUMAN | RPS10P5 | 20.11 | 3.3483 |
| 2 | 2 | sp\|Q9UJS0\|CMC2_HUMAN | SLC25A13 | 74.13 | 3.345 |
| 2 | 2 | sp\|Q6PK04\|CC137_HUMAN | CCDC137 | 33.21 | 3.344 |
| 2 | 2 | sp\|Q5T280\|CI114_HUMAN | SPOUT1 | 41.98 | 3.3243 |
| 2 | 2 | sp\|Q9UJV9\|DDX41_HUMAN | DDX41 | 69.79 | 3.3239 |
| 2 | 2 | sp\|Q9GZS3\|WDR61_HUMAN | WDR61 | 33.56 | 3.3203 |
| 2 | 2 | sp\|Q13084\|RM28_HUMAN | MRPL28 | 30.14 | 3.314 |
| 2 | 2 | sp\|Q9NPF5\|DMAP1_HUMAN | DMAP1 | 52.96 | 3.3061 |
| 2 | 2 | sp\|P82933\|RT09_HUMAN | MRPS9 | 45.81 | 3.2952 |
| 2 | 2 | sp\|Q9UGL1\|KDM5B_HUMAN | KDM5B | 175.54 | 3.2822 |
| 2 | 2 | sp\|Q5T3J3\|LRIF1_HUMAN | LRIF1 | 84.52 | 3.2788 |
| 2 | 2 | sp\|Q9GZR7\|DDX24_HUMAN | DDX24 | 96.27 | 3.2775 |
| 2 | 2 | sp\|P62304\|RUXE_HUMAN | SNRPE | 10.8 | 3.2748 |
| 2 | 2 | sp\|Q8N0S6\|CENPL_HUMAN | CENPL | 38.97 | 3.245 |
| 2 | 2 | sp\|Q14146\|URB2_HUMAN | URB2 | 170.43 | 3.1984 |
| 2 | 2 | sp\|Q02978\|M2OM_HUMAN | SLC25A11 | 34.04 | 3.1901 |
| 2 | 2 | sp\|Q9H6R0\|DHX33_HUMAN | DHX33 | 78.82 | 3.1845 |
| 2 | 2 | sp\|Q9HCD5\|NCOA5_HUMAN | NCOA5 | 65.5 | 3.1842 |
| 2 | 2 | sp\|Q86WX3\|AROS_HUMAN | RPS19BP1 | 15.42 | 3.1782 |
| 2 | 2 | sp\|Q9H4L4\|SENP3_HUMAN | SENP3 | 64.97 | 3.1604 |
| 2 | 2 | sp\|P61313\|RL15_HUMAN | RPL15 | 24.13 | 3.1313 |
| 2 | 2 | sp\|Q9BQF6\|SENP7_HUMAN | SENP7 | 119.58 | 3.1128 |
| 2 | 2 | sp\|P38646\|GRP75_HUMAN | HSPA9 | 73.63 | 3.1102 |
| 2 | 2 | sp\|Q5SY16\|NOL9_HUMAN | NOL9 | 79.27 | 3.107 |
| 2 | 2 | sp\|P07197\|NFM_HUMAN | NEFM | 102.41 | 3.1027 |
| 2 | 2 | sp\|P31942\|HNRH3_HUMAN | HNRNPH3 | 36.9 | 3.0985 |
| 2 | 2 | sp\|Q9Y265\|RUVB1_HUMAN | RUVBL1 | 50.2 | 3.0511 |
| 2 | 2 | sp\|Q9Y3B9\|RRP15_HUMAN | RRP15 | 31.46 | 3.0423 |
| 2 | 2 | sp\|Q9NSI2\|F207A_HUMAN | FAM207A | 25.44 | 3.0287 |
| 2 | 2 | sp\|Q6IQ32\|ADNP2_HUMAN | ADNP2 | 122.75 | 3.0046 |
| 2 | 2 | sp\|P55081\|MFAP1_HUMAN | MFAP1 | 51.93 | 2.9802 |
| 2 | 2 | sp\|P30050\|RL12_HUMAN | RPL12 | 17.81 | 2.9606 |
| 2 | 2 | sp\|O00257\|CBX4_HUMAN | CBX4 | 61.33 | 2.9486 |
| 2 | 2 | sp\|Q9BW27\|NUP85_HUMAN | NUP85 | 74.97 | 2.9405 |
| 2 | 2 | sp\|P83731\|RL24_HUMAN | RPL24 | 17.77 | 2.9294 |
| 2 | 2 | sp\|O95983\|MBD3_HUMAN | MBD3 | 32.82 | 2.9113 |
| 2 | 2 | sp\|P06576\|ATPB_HUMAN | ATP5B | 56.52 | 2.9104 |
| 2 | 2 | sp\|O75530\|EED_HUMAN | EED | 50.17 | 2.8773 |
| 2 | 2 | sp\|Q8NAV1\|PR38A_HUMAN | PRPF38A | 37.45 | 2.8489 |
| 2 | 2 | sp\|O60318\|GANP_HUMAN | MCM3AP | 218.27 | 2.8449 |
| 2 | 2 | sp\|Q02878\|RL6_HUMAN | RPL6 | 32.71 | 2.7827 |
| 2 | 2 | sp\|O94805\|ACL6B_HUMAN | ACTL6B | 46.85 | 2.773 |
| 2 | 2 | sp\|P08621\|RU17_HUMAN | SNRNP70 | 51.53 | 2.7581 |
| 2 | 2 | tr\|A0A096LNW1\|A0A096LNW1_HUMAN | ATRX | 106.9 | 2.7254 |
| 2 | 2 | sp\|P62318\|SMD3_HUMAN | SNRPD3 | 13.91 | 2.7076 |
| 2 | 2 | sp\|Q6P0N0\|M18BP_HUMAN | MIS18BP1 | 129.01 | 2.7046 |
| 2 | 2 | sp\|P13010\|XRCC5_HUMAN | XRCC5 | 82.65 | 2.6965 |
| 2 | 2 | sp\|P30414\|NKTR_HUMAN | NKTR | 165.58 | 2.6914 |
| 2 | 2 | sp\|P08574\|CY1_HUMAN | CYC1 | 35.4 | 2.6877 |
| 2 | 2 | sp\|Q9P1Y6\|PHRF1_HUMAN | PHRF1 | 178.56 | 2.5945 |
| 2 | 2 | sp\|Q9NW13\|RBM28_HUMAN | RBM28 | 85.68 | 2.5606 |
| 2 | 2 | sp\|P18847\|ATF3_HUMAN | ATF3 | 20.56 | 2.5504 |
| 2 | 2 | sp\|Q9UQ88\|CD11A_HUMAN | CDK11A | 91.31 | 2.4514 |
| 2 | 2 | sp\|Q9Y483\|MTF2_HUMAN | MTF2 | 67.06 | 2.3506 |
| 2 | 2 | sp\|Q9H6R4\|NOL6_HUMAN | NOL6 | 127.51 | 2.2599 |
| 2 | 2 | sp\|O00148\|DX39A_HUMAN | DDX39A | 49.1 | 2.1977 |
| 2 | 2 | sp\|Q9P2K5\|MYEF2_HUMAN | MYEF2 | 64.08 | 2.1848 |
| 1 | 2 | tr\|Q68E03\|Q68E03_HUMAN | DKFZp686L22104 | 30.92 | 5.1845 |
| 1 | 2 | sp\|Q16576\|RBBP7_HUMAN | RBBP7 | 47.79 | 3.3349 |
| 1 | 2 | sp\|Q9BYX7\|ACTBM_HUMAN | POTEKP | 41.99 | 3.1033 |
| 1 | 2 | sp\|P46777\|RL5_HUMAN | RPL5 | 34.34 | 2.8247 |
| 1 | 1 | sp\|O95299\|NDUAA_HUMAN | NDUFA10 | 40.72 | 5.8592 |
| 1 | 1 | tr\|A0A0M3HER2\|A0A0M3HER2_HUMAN | CENPV | 18.8 | 5.626 |
| 1 | 1 | sp\|P04280\|PRP1_HUMAN | PRB1 | 38.52 | 5.4758 |
| 1 | 1 | tr\|Q53GL6\|Q53GL6_HUMAN | RALY | 32.53 | 5.4233 |
| 1 | 1 | tr\|S4R341\|S4R341_HUMAN | NOLC1 | 8.05 | 5.3679 |
| 1 | 1 | sp\|Q9NW64\|RBM22_HUMAN | RBM22 | 46.87 | 5.1218 |
| 1 | 1 | sp\|Q9BYD2\|RM09_HUMAN | MRPL9 | 30.22 | 5.0825 |
| 1 | 1 | sp\|P46782\|RS5_HUMAN | RPS5 | 22.86 | 5.0071 |
| 1 | 1 | sp\|Q9NZM5\|GSCR2_HUMAN | GLTSCR2 | 54.36 | 4.9887 |
| 1 | 1 | sp\|Q92804\|RBP56_HUMAN | TAF15 | 61.79 | 4.88 |
| 1 | 1 | sp\|Q6NSZ9\|ZSC25_HUMAN | ZSCAN25 | 61.44 | 4.8752 |
| 1 | 1 | sp\|P16989\|YBOX3_HUMAN | YBX3 | 40.07 | 4.8301 |
| 1 | 1 | tr\|Q6IPH7\|Q61PH7_HUMAN | RPL14 | 23.77 | 4.7072 |
| 1 | 1 | sp\|Q9BQ04\|RBM4B_HUMAN | RBM4B | 40.12 | 4.7065 |
| 1 | 1 | sp\|Q9HCK1\|ZDBF2_HUMAN | ZDBF2 | 265.45 | 4.7004 |
| 1 | 1 | sp\|Q5T653\|RM02_HUMAN | MRPL2 | 33.28 | 4.6443 |
| 1 | 1 | sp\|P62136\|PP1A_HUMAN | PPP1CA | 37.49 | 4.5999 |
| 1 | 1 | sp\|Q92782\|DPF1_HUMAN | DPF1 | 42.47 | 4.5393 |
| 1 | 1 | sp\|Q13151\|ROAO_HUMAN | HNRNPA0 | 30.82 | 4.5336 |
| 1 | 1 | sp\|P35232\|PHB_HUMAN | PHB | 29.79 | 4.5324 |
| 1 | 1 | sp\|Q9BTC0\|DIDO1_HUMAN | DIDO1 | 243.72 | 4.5276 |
| 1 | 1 | sp\|Q9Y230\|RUVB2_HUMAN | RUVBL2 | 51.12 | 4.517 |
| 1 | 1 | sp\|P82673\|RT35_HUMAN | MRPS35 | 36.82 | 4.5017 |
| 1 | 1 | sp\|Q99567\|NUP88_HUMAN | NUP88 | 83.49 | 4.4729 |
| 1 | 1 | sp\|P05412\|JUN_HUMAN | JUN | 35.65 | 4.4638 |
| 1 | 1 | sp\|Q8NDX5\|PHC3_HUMAN | PHC3 | 106.1 | 4.4539 |
| 1 | 1 | sp\|P50914\|RL14_HUMAN | RPL14 | 23.42 | 4.3217 |
| 1 | 1 | sp\|O60506\|HNRPQ_HUMAN | SYNCRIP | 69.56 | 4.2631 |
| 1 | 1 | sp\|Q14974\|IMB1_HUMAN | KPNB1 | 97.11 | 4.2381 |
| 1 | 1 | sp\|P62913\|RL11_HUMAN | RPL11 | 20.24 | 4.2253 |
| 1 | 1 | sp\|O95391\|SLU7_HUMAN | SLU7 | 68.34 | 4.215 |
| 1 | 1 | sp\|P09234\|RU1C_HUMAN | SNRPC | 17.38 | 4.2105 |
| 1 | 1 | tr\|A8K7N0\|A8K7N0_HUMAN | | 23.63 | 4.193 |
| 1 | 1 | tr\|B2RC06\|B2RC06_HUMAN | | 39.25 | 4.162 |
| 1 | 1 | sp\|Q8TBK6\|ZCH10_HUMAN | ZCCHC10 | 20.95 | 4.1487 |
| 1 | 1 | sp\|Q9UBB5\|MBD2_HUMAN | MBD2 | 43.23 | 4.1327 |
| 1 | 1 | IGKC_MOUSE | | 11.77 | 4.1063 |
| 1 | 1 | sp\|P37198\|NUP62_HUMAN | NUP62 | 53.22 | 4.0927 |
| 1 | 1 | sp\|P35579\|MYH9_HUMAN | MYH9 | 226.39 | 4.0738 |
| 1 | 1 | sp\|P22695\|QCR2_HUMAN | UQCRC2 | 48.41 | 4.0303 |
| 1 | 1 | sp\|P07355\|ANXA2_HUMAN | ANXA2 | 38.58 | 4.0038 |
| 1 | 1 | sp\|O15381\|NVL_HUMAN | NVL | 94.99 | 3.9989 |
| 1 | 1 | sp\|Q07157\|ZO1_HUMAN | TJP1 | 195.34 | 3.9914 |
| 1 | 1 | sp\|Q9Y3C1\|NOP16_HUMAN | NOP16 | 21.18 | 3.9883 |
| 1 | 1 | sp\|Q9Y625\|GPC6_HUMAN | GPC6 | 62.69 | 3.9801 |
| 1 | 1 | tr\|B2RWN5\|B2RWN5_HUMAN | HEATR1 | 242.11 | 3.9638 |
| 1 | 1 | sp\|Q15910\|EZH2_HUMAN | EZH2 | 85.31 | 3.9454 |
| 1 | 1 | sp\|Q9NQV6\|PRD10_HUMAN | PRDM10 | 130.05 | 3.9287 |
| 1 | 1 | sp\|Q8TF76\|HASP_HUMAN | GSG2 | 88.44 | 3.8857 |
| 1 | 1 | sp\|Q9Y3B4\|SF3B6_HUMAN | SF3B6 | 14.58 | 3.8364 |
| 1 | 1 | sp\|P62314\|SMD1_HUMAN | SNRPD1 | 13.27 | 3.8351 |
| 1 | 1 | tr\|Q6FI97\|Q6FI97_HUMAN | BAF53A | 47.35 | 3.8176 |
| 1 | 1 | sp\|Q9P013\|CWC15_HUMAN | CWC15 | 26.61 | 3.8025 |
| 1 | 1 | sp\|P62899\|RL31_HUMAN | RPL31 | 14.45 | 3.7783 |
| 1 | 1 | sp\|P82663\|RT25_HUMAN | MRPS25 | 20.1 | 3.7565 |
| 1 | 1 | sp\|P35249\|RFC4_HUMAN | RFC4 | 39.66 | 3.7202 |
| 1 | 1 | sp\|P49759\|CLK1_HUMAN | CLK1 | 57.25 | 3.7089 |
| 1 | 1 | sp\|Q9NVH1\|DJC11_HUMAN | DNAJC11 | 63.24 | 3.7074 |
| 1 | 1 | sp\|Q9UHR5\|S30BP_HUMAN | SAP30BP | 33.85 | 3.691 |
| 1 | 1 | tr\|B0UZZ8\|B0UZZ8_HUMAN | C6orf11 | 68 | 3.6905 |
| 1 | 1 | sp\|Q8N9T8\|KRI1_HUMAN | KRI1 | 82.55 | 3.6636 |
| 1 | 1 | sp\|P51991\|ROA3_HUMAN | HNRNPA3 | 39.57 | 3.6618 |
| 1 | 1 | sp\|O14880\|MGST3_HUMAN | MGST3 | 16.51 | 3.6611 |
| 1 | 1 | tr\|Q05CW7\|Q05CW7_HUMAN | NAT10 | 62.35 | 3.6602 |
| 1 | 1 | sp\|O75934\|SPF27_HUMAN | BCAS2 | 26.11 | 3.6364 |
| 1 | 1 | sp\|Q8NHW5\|RLA0L_HUMAN | RPLP0P6 | 34.34 | 3.6321 |
| 1 | 1 | sp\|Q9NQ50\|RM40_HUMAN | MRPL40 | 24.48 | 3.629 |
| 1 | 1 | sp\|P12235\|ADT1_HUMAN | SLC25A4 | 33.04 | 3.6148 |
| 1 | 1 | sp\|O14519\|CDKA1_HUMAN | CDK2AP1 | 12.36 | 3.6134 |
| 1 | 1 | sp\|Q96EP5\|DAZP1_HUMAN | DAZAP1 | 43.36 | 3.6108 |
| 1 | 1 | sp\|Q9NYK5\|RM39_HUMAN | MRPL39 | 38.69 | 3.5747 |
| 1 | 1 | sp\|P05023\|AT1A1_HUMAN | ATP1A1 | 112.82 | 3.565 |
| 1 | 1 | sp\|P35250\|RFC2_HUMAN | RFC2 | 39.13 | 3.526 |
| 1 | 1 | sp\|Q9Y676\|RT18B_HUMAN | MRPS18B | 29.38 | 3.5192 |
| 1 | 1 | sp\|O94832\|MYO1D_HUMAN | MYO1D | 116.13 | 3.5141 |
| 1 | 1 | sp\|O60762\|DPM1_HUMAN | DPM1 | 29.62 | 3.5123 |
| 1 | 1 | sp\|Q9UBU9\|NXF1_HUMAN | NXF1 | 70.14 | 3.5123 |
| 1 | 1 | sp\|Q96JM3\|CHAP1_HUMAN | CHAMP1 | 89.04 | 3.5122 |
| 1 | 1 | sp\|Q9BYD3\|RM04_HUMAN | MRPL4 | 34.9 | 3.4916 |
| 1 | 1 | sp\|Q9H7H0\|MET17_HUMAN | METTL17 | 50.7 | 3.483 |
| 1 | 1 | sp\|Q9NX24\|NHP2_HUMAN | NHP2 | 17.19 | 3.472 |
| 1 | 1 | tr\|Q562V5\|Q562V5_HUMAN | ACT | 11.42 | 3.4708 |
| 1 | 1 | sp\|Q9Y5Q9\|TF3C3_HUMAN | GTF3C3 | 101.21 | 3.4705 |
| 1 | 1 | sp\|Q9ULU4\|PKCB1_HUMAN | ZMYND8 | 131.61 | 3.4689 |
| 1 | 1 | sp\|Q9HBE1\|PATZ1_HUMAN | PATZ1 | 74.01 | 3.4686 |
| 1 | 1 | sp\|P51571\|SSRD_HUMAN | SSR4 | 18.99 | 3.4601 |
| 1 | 1 | sp\|Q06587\|RING1_HUMAN | RING1 | 42.4 | 3.4513 |
| 1 | 1 | sp\|O14795\|UN13B_HUMAN | UNC13B | 180.56 | 3.4394 |
| 1 | 1 | sp\|P33992\|MCM5_HUMAN | MCM5 | 82.23 | 3.3963 |
| 1 | 1 | sp\|Q13148\|TADBP_HUMAN | TARDBP | 44.71 | 3.3906 |
| 1 | 1 | sp\|Q8WY36\|BBX_HUMAN | BBX | 105.06 | 3.3426 |
| 1 | 1 | sp\|Q86X18\|CS068_HUMAN | C19orf68 | 70.03 | 3.3403 |
| 1 | 1 | tr\|B7Z8Y3\|B7Z8Y3_HUMAN | | 106.95 | 3.3372 |
| 1 | 1 | sp\|Q8NDF8\|PAPD5_HUMAN | PAPD5 | 63.23 | 3.3133 |
| 1 | 1 | sp\|P32119\|PRDX2_HUMAN | PRDX2 | 21.88 | 3.312 |
| 1 | 1 | sp\|Q96SK2\|TM209_HUMAN | TMEM209 | 62.88 | 3.3026 |
| 1 | 1 | tr\|D3DTH7\|D3DTH7_HUMAN | MYO1C | 98.87 | 3.2974 |
| 1 | 1 | tr\|B4DR34\|B4DR34_HUMAN | | 36.86 | 3.2621 |
| 1 | 1 | sp\|Q9NP66\|HM20A_HUMAN | HMG20A | 40.12 | 3.2532 |
| 1 | 1 | sp\|O75955\|FLOT1_HUMAN | FLOT1 | 47.33 | 3.2455 |
| 1 | 1 | sp\|Q96GN5\|CDA7L_HUMAN | CDCA7L | 52.17 | 3.2347 |
| 1 | 1 | sp\|Q7Z6E9\|RBBP6_HUMAN | RBBP6 | 201.44 | 3.2342 |
| 1 | 1 | sp\|A8CG34\|P121C_HUMAN | POM121C | 124.98 | 3.2079 |
| 1 | 1 | sp\|P41208\|CETN2_HUMAN | CETN2 | 19.73 | 3.2075 |
| 1 | 1 | sp\|P62829\|RL23_HUMAN | RPL23 | 14.86 | 3.2074 |
| 1 | 1 | sp\|Q9NXE4\|NSMA3_HUMAN | SMPD4 | 93.29 | 3.2044 |
| 1 | 1 | sp\|Q86Y91\|KI18B_HUMAN | KIF18B | 94.16 | 3.1821 |
| 1 | 1 | sp\|Q6DRA6\|H2B2D_HUMAN | HIST2H2BD | 18.01 | 3.1771 |
| 1 | 1 | sp\|P62263\|RS14_HUMAN | RPS14 | 16.26 | 3.1597 |
| 1 | 1 | sp\|Q9UHX1\|PUF60_HUMAN | PUF60 | 59.84 | 3.1255 |
| 1 | 1 | sp\|E9PRG8\|CK098_HUMAN | C11orf98 | 13.79 | 3.1088 |
| 1 | 1 | sp\|Q75QN2\|INT8_HUMAN | INTS8 | 113.02 | 3.1011 |
| 1 | 1 | sp\|Q58FF8\|H90B2_HUMAN | HSP90AB2P | 44.32 | 3.0769 |
| 1 | 1 | sp\|O75477\|ERLN1_HUMAN | ERLIN1 | 38.9 | 3.0624 |
| 1 | 1 | sp\|Q15843\|NEDD8_HUMAN | NEDD8 | 9.07 | 3.0468 |
| 1 | 1 | sp\|Q15365\|PCBP1_HUMAN | PCBP1 | 37.47 | 3.0189 |
| 1 | 1 | sp\|P0DMR1\|HNRC4_HUMAN | HNRNPCL4 | 32.01 | 3.0131 |
| 1 | 1 | sp\|Q9ULV3\|CIZ1_HUMAN | CIZ1 | 99.98 | 3.0112 |
| 1 | 1 | sp\|P00558\|PGK1_HUMAN | PGK1 | 44.59 | 3.0094 |
| 1 | 1 | sp\|Q8TE59\|ATS19_HUMAN | ADAMTS19 | 133.96 | 3.0037 |
| 1 | 1 | sp\|Q8NAP3\|ZBT38_HUMAN | ZBTB38 | 134.17 | 2.9639 |
| 1 | 1 | KV2A7_MOUSE | | 12.27 | 2.9512 |
| 1 | 1 | sp\|Q8IXKO\|PHC2_HUMAN | PHC2 | 90.66 | 2.9338 |
| 1 | 1 | sp\|O60341\|KDM1A_HUMAN | KDM1A | 92.84 | 2.9303 |
| 1 | 1 | sp\|Q9Y6F7\|CDY2_HUMAN | CDY2A | 60.49 | 2.9091 |
| 1 | 1 | sp\|O95235\|KI20A_HUMAN | KIF20A | 100.22 | 2.8771 |
| 1 | 1 | sp\|Q9UJZ1\|STML2_HUMAN | STOML2 | 38.51 | 2.8728 |
| 1 | 1 | sp\|P04843\|RPN1_HUMAN | RPN1 | 68.53 | 2.868 |
| 1 | 1 | sp\|Q9BVA1\|TBB2B_HUMAN | TUBB2B | 49.92 | 2.8339 |
| 1 | 1 | sp\|P43246\|MSH2_HUMAN | MSH2 | 104.68 | 2.8127 |
| 1 | 1 | sp\|Q8WUB8\|PHF10_HUMAN | PHF10 | 56.02 | 2.7847 |
| 1 | 1 | sp\|Q5JVF3\|PCID2_HUMAN | PCID2 | 46 | 2.7464 |
| 1 | 1 | sp\|Q9Y3Y2\|CHTOP_HUMAN | CHTOP | 26.38 | 2.714 |
| 1 | 1 | sp\|Q9Y2X9\|ZN281_HUMAN | ZNF281 | 96.85 | 2.6924 |
| 1 | 1 | sp\|Q53GQ0\|DHB12_HUMAN | HSD17B12 | 34.3 | 2.6858 |
| 1 | 1 | tr\|A0A024R5M9\|A0A024R5M9_HUMAN | NUMA1 | 236.37 | 2.6796 |
| 1 | 1 | sp\|P17010\|ZFX_HUMAN | ZFX | 90.46 | 2.6696 |
| 1 | 1 | sp\|P54652\|HSP72_HUMAN | HSPA2 | 69.98 | 2.6579 |
| 1 | 1 | sp\|Q9UKD2\|MRT4_HUMAN | MRTO4 | 27.54 | 2.6442 |
| 1 | 1 | sp\|P12273\|PIP_HUMAN | PIP | 16.56 | 2.6385 |
| 1 | 1 | sp\|Q14151\|SAFB2_HUMAN | SAFB2 | 107.41 | 2.6256 |
| 1 | 1 | sp\|Q9UHA3\|RLP24_HUMAN | RSL24D1 | 19.61 | 2.6188 |
| 1 | 1 | sp\|Q5H9F3\|BCORL_HUMAN | BCORL1 | 182.41 | 2.5997 |
| 1 | 1 | sp\|Q8NDD1\|CA131_HUMAN | C1orf131 | 32.75 | 2.5842 |
| 1 | 1 | sp\|Q9NWU5\|RM22_HUMAN | MRPL22 | 23.63 | 2.5746 |
| 1 | 1 | sp\|Q9NZ01\|TECR_HUMAN | TECR | 36.01 | 2.5526 |
| 1 | 1 | sp\|Q16352\|AINX_HUMAN | INA | 55.36 | 2.5377 |
| 1 | 1 | sp\|O15504\|NUPL2_HUMAN | NUPL2 | 44.84 | 2.5352 |
| 1 | 1 | sp\|Q9NR22\|ANM8_HUMAN | PRMT8 | 45.26 | 2.5291 |
| 1 | 1 | sp\|P68871\|HBB_HUMAN | HBB | 15.99 | 2.5078 |
| 1 | 1 | sp\|Q5SNT2\|TM201_HUMAN | TMEM201 | 72.19 | 2.4788 |
| 1 | 1 | sp\|P46776\|RL27A_HUMAN | RPL27A | 16.55 | 2.3924 |
| 1 | 1 | sp\|P13639\|EF2_HUMAN | EEF2 | 95.28 | 2.3879 |
| 1 | 1 | sp\|Q8NDX1\|PSD4_HUMAN | PSD4 | 116.18 | 2.347 |
| 1 | 1 | sp\|P62861\|RS30_HUMAN | FAU | 6.64 | 2.3442 |
| 1 | 1 | sp\|P13804\|ETFA_HUMAN | ETFA | 35.06 | 2.3145 |
| 1 | 1 | sp\|Q9NV06\|DCA13_HUMAN | DCAF13 | 51.37 | 2.3115 |
| 1 | 1 | sp\|O75190\|DNJB6_HUMAN | DNAJB6 | 36.06 | 2.3041 |
| 1 | 1 | sp\|P62857\|RS28_HUMAN | RPS28 | 7.84 | 2.2822 |
| 1 | 1 | sp\|Q9NY93\|DDX56_HUMAN | DDX56 | 61.55 | 2.2165 |
| 1 | 1 | sp\|P51784\|UBP11_HUMAN | USP11 | 109.75 | 2.2148 |
| 1 | 1 | sp\|Q9NVH2\|INT7_HUMAN | INTS7 | 106.77 | 2.2143 |
| 1 | 1 | sp\|O60673\|REV3L_HUMAN | REV3L | 352.55 | 2.2069 |
| 1 | 1 | sp\|Q9P015\|RM15_HUMAN | MRPL15 | 33.4 | 2.2064 |
| 1 | 1 | sp\|P61353\|RL27_HUMAN | RPL27 | 15.79 | 2.1707 |
| 1 | 1 | sp\|O75323\|NIPS2_HUMAN | NIPSNAP2 | 33.72 | 2.1553 |
| 1 | 1 | tr\|Q9UMG4\|Q9UMG4_HUMAN | hANK1 | 3.65 | 2.1374 |
| 1 | 1 | sp\|Q8IXM6\|NRM_HUMAN | NRM | 29.36 | 2.1313 |

TABLE 5B
HA-SS18SSX1_NE_peptides

| Unique | Total | reference | Gene Symbol | MWT(kDa) | AVG |
|---|---|---|---|---|---|
| 48 | 308 | sp\|P51532\|SMCA4_HUMAN | SMARCA4 | 184.53 | 2.8323 |
| 71 | 221 | sp\|O14497\|ARI1A_HUMAN | ARID1A | 241.89 | 3.2897 |
| 60 | 221 | sp\|P51531\|SMCA2_HUMAN | SMARCA2 | 181.17 | 2.9484 |
| 37 | 213 | sp\|Q92922\|SMRC1_HUMAN | SMARCC1 | 122.79 | 2.94 |
| 51 | 174 | sp\|Q8TAQ2\|SMRC2_HUMAN | SMARCC2 | 132.8 | 2.952 |
| 52 | 108 | sp\|Q8NFD5\|ARI1B_HUMAN | ARID1B | 235.97 | 3.1352 |
| 27 | 94 | sp\|Q96GM5\|SMRD1_HUMAN | SMARCD1 | 58.2 | 3.4459 |
| 78 | 89 | sp\|P78527\|PRKDC_HUMAN | PRKDC | 468.79 | 3.2311 |
| 34 | 89 | sp\|Q9NZM4\|BICRA_HUMAN | BICRA | 158.39 | 3.2316 |
| 26 | 77 | sp\|Q969G3\|SMCE1_HUMAN | SMARCE1 | 46.62 | 3.4283 |
| 28 | 75 | sp\|Q6STE5\|SMRD3_HUMAN | SMARCD3 | 54.98 | 2.9487 |
| 19 | 74 | sp\|O96019\|ACL6A_HUMAN | ACTL6A | 47.43 | 3.1679 |
| 21 | 46 | sp\|Q92925\|SMRD2_HUMAN | SMARCD2 | 58.88 | 3.1179 |
| 20 | 46 | sp\|P49411\|EFTU_HUMAN | TUFM | 49.51 | 2.681 |
| 10 | 46 | sp\|P62736\|ACTA_HUMAN | ACTA2 | 41.98 | 2.4533 |
| 16 | 45 | sp\|Q12824\|SNF5_HUMAN | SMARCB1 | 44.11 | 2.9132 |
| 7 | 41 | sp\|P63261\|ACTG_HUMAN | ACTG1 | 41.77 | 3.2375 |
| 24 | 40 | sp\|Q9H8M2\|BRD9_HUMAN | BRD9 | 66.96 | 3.3845 |
| 24 | 39 | sp\|P25705\|ATPA_HUMAN | ATP5A1 | 59.71 | 3.347 |
| 14 | 35 | sp\|Q92785\|REQU_HUMAN | DPF2 | 44.13 | 3.6206 |
| 23 | 34 | sp\|Q9UJS0\|CMC2_HUMAN | SLC25A13 | 74.13 | 3.1068 |
| 14 | 31 | sp\|P12236\|ADT3_HUMAN | SLC25A6 | 32.85 | 2.5554 |
| 15 | 30 | sp\|P52272\|HNRPM_HUMAN | HNRNPM | 77.46 | 3.2894 |
| 22 | 25 | sp\|P06576\|ATPB_HUMAN | ATP5B | 56.52 | 3.6483 |
| 24 | 24 | sp\|O75643\|U520_HUMAN | SNRNP200 | 244.35 | 3.1225 |
| 23 | 23 | sp\|Q14980\|NUMA1_HUMAN | NUMA1 | 238.12 | 3.7937 |
| 23 | 23 | sp\|Q14204\|DYHC1_HUMAN | DYNC1H1 | 532.07 | 3.2127 |
| 18 | 23 | sp\|P05023\|AT1A1_HUMAN | ATP1A1 | 112.82 | 3.7257 |
| 15 | 23 | sp\|O95831\|AIFM1_HUMAN | AIFM1 | 66.86 | 3.2173 |
| 12 | 23 | sp\|P50402\|EMD_HUMAN | EMD | 28.98 | 2.7585 |
| 12 | 22 | sp\|P68371\|TBB4B_HUMAN | TUBB4B | 49.8 | 3.5227 |
| 10 | 22 | sp\|Q00325\|MPCP_HUMAN | SLC25A3 | 40.07 | 2.6968 |
| 16 | 21 | sp\|Q08211\|DHX9_HUMAN | DHX9 | 140.87 | 2.9913 |
| 14 | 21 | sp\|P11021\|GRP78_HUMAN | HSPA5 | 72.29 | 3.2487 |
| 2 | 21 | sp\|Q15532\|SSXT_HUMAN | SS18 | 45.9 | 2.4719 |
| 15 | 20 | sp\|P38646\|GRP75_HUMAN | HSPA9 | 73.63 | 3.5062 |
| 7 | 20 | sp\|Q4VC05\|BCL7A_HUMAN | BCL7A | 22.8 | 3.3097 |
| 18 | 19 | sp\|P52701\|MSH6_HUMAN | MSH6 | 152.69 | 3.4726 |
| 11 | 19 | sp\|Q6AI39\|BICRL_HUMAN | BICRAL | 115.01 | 3.2661 |
| 9 | 19 | sp\|P68104\|EF1A1_HUMAN | EEF1A1 | 50.11 | 3.1829 |
| 16 | 18 | sp\|P09874\|PARP1_HUMAN | PARP1 | 113.01 | 3.0283 |
| 16 | 18 | sp\|Q10570\|CPSF1_HUMAN | CPSF1 | 160.78 | 2.7977 |
| 16 | 17 | sp\|P20700\|LMNB1_HUMAN | LMNB1 | 66.37 | 3.3547 |
| 15 | 17 | sp\|Q16891\|MIC60_HUMAN | IMMT | 83.63 | 3.5711 |
| 16 | 16 | sp\|Q8N1F7\|NUP93_HUMAN | NUP93 | 93.43 | 3.0683 |
| 13 | 16 | sp\|P40939\|ECHA_HUMAN | HADHA | 82.95 | 3.3237 |
| 13 | 16 | sp\|O00567\|NOP56_HUMAN | NOP56 | 66.01 | 3.1798 |
| 9 | 16 | sp\|P62805\|H4_HUMAN | HIST1H4A | 11.36 | 3.0197 |
| 14 | 15 | sp\|P42704\|LPPRC_HUMAN | LRPPRC | 157.81 | 3.2864 |
| 13 | 15 | sp\|Q9UJV9\|DDX41_HUMAN | DDX41 | 69.79 | 2.6016 |
| 5 | 15 | sp\|Q71DI3\|H32_HUMAN | HIST2H3A | 15.38 | 1.934 |
| 14 | 14 | sp\|Q6P2Q9\|PRP8_HUMAN | PRPF8 | 273.43 | 2.9384 |
| 13 | 14 | sp\|Q9NVI7\|ATD3A_HUMAN | ATAD3A | 71.32 | 3.1974 |
| 13 | 14 | sp\|Q92621\|NU205_HUMAN | NUP205 | 227.78 | 3.1896 |
| 7 | 14 | sp\|Q8WUZ0\|BCL7C_HUMAN | BCL7C | 23.45 | 3.5497 |
| 13 | 13 | sp\|Q9Y4W6\|AFG32_HUMAN | AFG3L2 | 88.53 | 3.0599 |
| 13 | 13 | sp\|Q9BUQ8\|DDX23_HUMAN | DDX23 | 95.52 | 3.0451 |
| 12 | 13 | sp\|Q9C0J8\|WDR33_HUMAN | WDR33 | 145.8 | 3.3842 |
| 11 | 13 | sp\|Q96A33\|CCD47_HUMAN | CCDC47 | 55.84 | 3.3429 |
| 9 | 13 | sp\|P43243\|MATR3_HUMAN | MATR3 | 94.56 | 2.9462 |
| 12 | 12 | sp\|P04844\|RPN2_HUMAN | RPN2 | 69.24 | 3.9026 |
| 12 | 12 | sp\|Q9Y5B6\|PAXB1_HUMAN | PAXBP1 | 104.74 | 3.5313 |
| 12 | 12 | sp\|A0FGR8\|ESYT2_HUMAN | ESYT2 | 102.29 | 3.287 |
| 10 | 12 | sp\|O75746\|CMC1_HUMAN | SLC25A12 | 74.71 | 3.0669 |
| 6 | 12 | sp\|P55795\|HNRH2_HUMAN | HNRNPH2 | 49.23 | 3.0612 |
| 5 | 12 | sp\|Q96KK5\|H2A1H_HUMAN | HIST1H2AH | 13.9 | 2.5583 |
| 2 | 12 | sp\|P05141\|ADT2_HUMAN | SLC25A5 | 32.83 | 3.4462 |
| 11 | 11 | sp\|Q15029\|U5S1_HUMAN | EFTUD2 | 109.37 | 3.6326 |
| 11 | 11 | sp\|Q02978\|M2OM_HUMAN | SLC25A11 | 34.04 | 3.5222 |
| 10 | 11 | sp\|Q9Y2X3\|NOP58_HUMAN | NOP58 | 59.54 | 4.2232 |
| 10 | 11 | sp\|Q7L0Y3\|MRRP1_HUMAN | TRMT10C | 47.32 | 2.9714 |
| 9 | 11 | sp\|P22695\|QCR2_HUMAN | UQCRC2 | 48.41 | 3.5992 |
| 9 | 11 | sp\|Q9BQE3\|TBA1C_HUMAN | TUBA1C | 49.86 | 3.3342 |
| 9 | 11 | sp\|Q53GQ0\|DHB12_HUMAN | HSD17B12 | 34.3 | 3.2769 |
| 9 | 11 | sp\|Q6UN15\|FIP1_HUMAN | FIP1L1 | 66.49 | 3.1953 |
| 9 | 11 | sp\|Q9H9B4\|SFXN1_HUMAN | SFXN1 | 35.6 | 3.0828 |
| 8 | 11 | sp\|O75306\|NDUS2_HUMAN | NDUFS2 | 52.51 | 3.1642 |
| 5 | 11 | sp\|P33778\|H2B1B_HUMAN | HIST1H2BB | 13.94 | 2.5844 |
| 10 | 10 | sp\|O14980\|XPO1_HUMAN | XPO1 | 123.31 | 3.5888 |
| 10 | 10 | sp\|Q6NUK1\|SCMC1_HUMAN | SLC25A24 | 53.32 | 3.4159 |
| 10 | 10 | sp\|P04181\|OAT_HUMAN | OAT | 48.5 | 3.4156 |
| 10 | 10 | sp\|Q96I99\|SUCB2_HUMAN | SUCLG2 | 46.48 | 3.3701 |
| 10 | 10 | sp\|Q86WJ1\|CHD1L_HUMAN | CHD1L | 100.92 | 3.2369 |
| 10 | 10 | sp\|P10809\|CH60_HUMAN | HSPD1 | 61.02 | 2.9893 |
| 10 | 10 | sp\|Q9UBB9\|TFP11_HUMAN | TFIP11 | 96.76 | 2.8601 |
| 10 | 10 | sp\|Q9BQG0\|MBB1A_HUMAN | MYBBP1A | 148.76 | 2.733 |
| 4 | 10 | sp\|Q16384\|SSX1_HUMAN | SSX1 | 21.92 | 3.3189 |
| 4 | 10 | IGH1M_MOUSE | lghg1 | 43.36 | 2.7234 |
| 9 | 9 | sp\|Q96TA2\|YMEL1_HUMAN | YME1L1 | 86.4 | 4.2777 |
| 9 | 9 | sp\|P30837\|AL1B1_HUMAN | ALDH1B1 | 57.17 | 4.1396 |
| 9 | 9 | sp\|P35251\|RFC1_HUMAN | RFC1 | 128.18 | 3.6089 |
| 9 | 9 | sp\|Q16822\|PCKGM_HUMAN | PCK2 | 70.68 | 3.2754 |
| 9 | 9 | sp\|Q9NTI5\|PDS5B_HUMAN | PDS5B | 164.56 | 2.6945 |
| 8 | 9 | sp\|P11142\|HSP7C_HUMAN | HSPA8 | 70.85 | 3.7908 |
| 8 | 9 | sp\|Q12769\|NU160_HUMAN | NUP160 | 162.02 | 3.4687 |
| 8 | 9 | sp\|Q15758\|AAAT_HUMAN | SLC1A5 | 56.56 | 3.2819 |
| 8 | 9 | sp\|P55084\|ECHB_HUMAN | HADHB | 51.26 | 3.1472 |
| 7 | 9 | sp\|P21796\|VDAC1_HUMAN | VDAC1 | 30.75 | 3.0601 |
| 7 | 9 | sp\|Q9P035\|HACD3_HUMAN | HACD3 | 43.13 | 2.84 |
| 6 | 9 | sp\|Q5T280\|CI114_HUMAN | SPOUT1 | 41.98 | 3.0837 |
| 6 | 9 | sp\|P22087\|FBRL_HUMAN | FBL | 33.76 | 2.914 |
| 6 | 9 | sp\|P36542\|ATPG_HUMAN | ATP5C1 | 32.98 | 2.6524 |
| 6 | 9 | sp\|Q9P2R7\|SUCB1_HUMAN | SUCLA2 | 50.29 | 2.518 |
| 8 | 8 | sp\|Q29RF7\|PDS5A_HUMAN | PDS5A | 150.73 | 3.8078 |
| 8 | 8 | sp\|P11310\|ACADM_HUMAN | ACADM | 46.56 | 3.2564 |
| 8 | 8 | sp\|Q9NUL7\|DDX28_HUMAN | DDX28 | 59.54 | 3.2067 |
| 8 | 8 | sp\|Q92841\|DDX17_HUMAN | DDX17 | 80.22 | 3.1678 |
| 8 | 8 | sp\|P50570\|DYN2_HUMAN | DNM2 | 98 | 3.0011 |
| 8 | 8 | sp\|Q5SRE5\|NU188_HUMAN | NUP188 | 195.92 | 2.437 |
| 7 | 8 | sp\|P16615\|AT2A2_HUMAN | ATP2A2 | 114.68 | 3.0714 |
| 7 | 8 | sp\|Q8N8A6\|DDX51_HUMAN | DDX51 | 72.41 | 3.0036 |
| 6 | 8 | sp\|Q15637\|SF01_HUMAN | SF1 | 68.29 | 3.1692 |
| 6 | 8 | sp\|P53985\|MOT1_HUMAN | SLC16A1 | 53.91 | 2.686 |
| 7 | 7 | sp\|Q53H12\|AGK_HUMAN | AGK | 47.11 | 3.4069 |
| 7 | 7 | sp\|O43175\|SERA_HUMAN | PHGDH | 56.61 | 3.249 |
| 7 | 7 | sp\|P45880\|VDAC2_HUMAN | VDAC2 | 31.55 | 3.0615 |
| 7 | 7 | sp\|P53621\|COPA_HUMAN | COPA | 138.26 | 3.0594 |
| 7 | 7 | sp\|P48047\|ATPO_HUMAN | ATP5O | 23.26 | 2.8331 |
| 7 | 7 | sp\|P46977\|STT3A_HUMAN | STT3A | 80.48 | 2.6162 |
| 6 | 7 | sp\|P42167\|LAP2B_HUMAN | TMPO | 50.64 | 3.4633 |
| 6 | 7 | sp\|O14983\|AT2A1_HUMAN | ATP2A1 | 110.18 | 3.3261 |
| 6 | 7 | sp\|Q96T37\|RBM15_HUMAN | RBM15 | 107.12 | 2.7759 |
| 6 | 6 | sp\|O75489\|NDUS3_HUMAN | NDUFS3 | 30.22 | 3.7176 |
| 6 | 6 | sp\|Q9NXE4\|NSMA3_HUMAN | SMPD4 | 93.29 | 3.5606 |
| 6 | 6 | sp\|Q92576\|PHF3_HUMAN | PHF3 | 229.34 | 3.519 |
| 6 | 6 | sp\|Q9Y2R4\|DDX52_HUMAN | DDX52 | 67.46 | 3.4811 |
| 6 | 6 | sp\|P38432\|COIL_HUMAN | COIL | 62.57 | 3.3461 |
| 6 | 6 | sp\|Q12931\|TRAP1_HUMAN | TRAP1 | 80.06 | 3.332 |
| 6 | 6 | sp\|O75367\|H2AY_HUMAN | H2AFY | 39.59 | 3.2768 |
| 6 | 6 | sp\|Q9P2I0\|CPSF2_HUMAN | CPSF2 | 88.43 | 3.2559 |
| 6 | 6 | sp\|Q96NB2\|SFXN2_HUMAN | SFXN2 | 36.21 | 3.2502 |
| 6 | 6 | sp\|P43246\|MSH2_HUMAN | MSH2 | 104.68 | 3.2395 |
| 6 | 6 | sp\|Q92616\|GCN1_HUMAN | GCN1 | 292.57 | 3.0917 |
| 6 | 6 | sp\|O60313\|OPA1_HUMAN | OPA1 | 111.56 | 3.0052 |
| 6 | 6 | sp\|Q6JQN1\|ACD10_HUMAN | ACAD10 | 118.76 | 2.8882 |
| 5 | 6 | sp\|P35250\|RFC2_HUMAN | RFC2 | 39.13 | 4.048 |
| 5 | 6 | sp\|P53618\|COPB_HUMAN | COPB1 | 107.07 | 3.5299 |
| 5 | 6 | sp\|Q969V3\|NCLN_HUMAN | NCLN | 62.93 | 3.1625 |
| 5 | 6 | sp\|P50213\|IDH3A_HUMAN | IDH3A | 39.57 | 3.0362 |
| 5 | 6 | sp\|P49590\|SYHM_HUMAN | HARS2 | 56.85 | 3.0295 |
| 5 | 6 | sp\|P53007\|TXTP_HUMAN | SLC25A1 | 33.99 | 2.6822 |
| 5 | 6 | sp\|Q96CS3\|FAF2_HUMAN | FAF2 | 52.59 | 2.5882 |
| 4 | 6 | sp\|P50416\|CPT1A_HUMAN | CPT1A | 88.31 | 3.1041 |
| 5 | 5 | sp\|Q9HC07\|TM165_HUMAN | TMEM165 | 34.88 | 4.4695 |
| 5 | 5 | sp\|Q9NVH2\|INT7_HUMAN | INTS7 | 106.77 | 3.8125 |
| 5 | 5 | sp\|P52597\|HNRPF_HUMAN | HNRNPF | 45.64 | 3.783 |
| 5 | 5 | sp\|Q9HCM4\|E41L5_HUMAN | EPB41L5 | 81.8 | 3.7077 |
| 5 | 5 | sp\|P45954\|ACDSB_HUMAN | ACADSB | 47.46 | 3.5191 |
| 5 | 5 | sp\|P35613\|BASI_HUMAN | BSG | 42.17 | 3.5115 |
| 5 | 5 | sp\|Q9NSE4\|SYIM_HUMAN | IARS2 | 113.72 | 3.5039 |
| 5 | 5 | sp\|Q9UKM7\|MA1B1_HUMAN | MAN1B1 | 79.53 | 3.3681 |
| 5 | 5 | sp\|P33993\|MCM7_HUMAN | MCM7 | 81.26 | 3.3539 |
| 5 | 5 | sp\|Q14683\|SMC1A_HUMAN | SMC1A | 143.14 | 3.3185 |
| 5 | 5 | sp\|Q3SY69\|AL1L2_HUMAN | ALDH1L2 | 101.68 | 3.3065 |
| 5 | 5 | sp\|Q8IXI1\|MIRO2_HUMAN | RHOT2 | 68.07 | 3.2378 |
| 5 | 5 | sp\|Q08945\|SSRP1_HUMAN | SSRP1 | 81.02 | 3.2282 |
| 5 | 5 | sp\|P55786\|PSA_HUMAN | NPEPPS | 103.21 | 3.17 |
| 5 | 5 | sp\|P18074\|ERCC2_HUMAN | ERCC2 | 86.85 | 3.1631 |
| 5 | 5 | sp\|Q96EY1\|DNJA3_HUMAN | DNAJA3 | 52.46 | 3.1378 |
| 5 | 5 | sp\|Q9UH62\|ARMX3_HUMAN | ARMCX3 | 42.47 | 2.9452 |
| 5 | 5 | sp\|P39656\|OST48_HUMAN | DDOST | 50.77 | 2.9053 |
| 5 | 5 | sp\|Q8NDT2\|RB15B_HUMAN | RBM15B | 97.15 | 2.8733 |
| 5 | 5 | sp\|Q8IXI2\|MIRO1_HUMAN | RHOT1 | 70.74 | 2.8581 |
| 5 | 5 | sp\|Q9Y305\|ACOT9_HUMAN | ACOT9 | 49.87 | 2.7301 |
| 5 | 5 | sp\|Q8IZL8\|PELP1_HUMAN | PELP1 | 119.62 | 2.691 |
| 5 | 5 | sp\|P49756\|RBM25_HUMAN | RBM25 | 100.12 | 2.6029 |
| 4 | 5 | sp\|O00411\|RPOM_HUMAN | POLRMT | 138.53 | 3.9144 |
| 4 | 5 | sp\|Q92784\|DPF3_HUMAN | DPF3 | 43.06 | 3.5795 |
| 4 | 5 | sp\|P31943\|HNRH1_HUMAN | HNRNPH1 | 49.2 | 3.2832 |
| 4 | 5 | sp\|P24539\|AT5F1_HUMAN | ATP5F1 | 28.89 | 3.0786 |
| 4 | 5 | sp\|P34931\|HS71L_HUMAN | HSPA1L | 70.33 | 3.0763 |
| 4 | 5 | sp\|Q14739\|LBR_HUMAN | LBR | 70.66 | 3.0611 |
| 4 | 5 | sp\|P11177\|ODPB_HUMAN | PDHB | 39.21 | 2.8626 |
| 3 | 5 | sp\|P40938\|RFC3_HUMAN | RFC3 | 40.53 | 2.7258 |
| 4 | 4 | sp\|O75600\|KBL_HUMAN | GCAT | 45.26 | 4.4371 |
| 4 | 4 | sp\|Q9UH99\|SUN2_HUMAN | SUN2 | 80.26 | 4.1201 |
| 4 | 4 | sp\|Q5T9A4\|ATD3B_HUMAN | ATAD3B | 72.53 | 4.0914 |
| 4 | 4 | sp\|Q49A26\|GLYR1_HUMAN | GLYR1 | 60.52 | 3.9554 |
| 4 | 4 | sp\|Q9BW27\|NUP85_HUMAN | NUP85 | 74.97 | 3.9325 |
| 4 | 4 | sp\|Q8TED0\|UTP15_HUMAN | UTP15 | 58.38 | 3.9212 |
| 4 | 4 | sp\|P28331\|NDUS1_HUMAN | NDUFS1 | 79.42 | 3.8974 |
| 4 | 4 | sp\|Q3ZCQ8\|TIM50_HUMAN | TIMM50 | 39.62 | 3.8468 |
| 4 | 4 | sp\|Q9BQE9\|BCL7B_HUMAN | BCL7B | 22.18 | 3.7012 |
| 4 | 4 | sp\|O14828\|SCAM3_HUMAN | SCAMP3 | 38.26 | 3.6839 |
| 4 | 4 | sp\|P0DMV9\|HS71B_HUMAN | HSPA1B | 70.01 | 3.6515 |
| 4 | 4 | sp\|Q14684\|RRP1B_HUMAN | RRP1B | 84.38 | 3.6515 |
| 4 | 4 | sp\|P35249\|RFC4_HUMAN | RFC4 | 39.66 | 3.6418 |
| 4 | 4 | sp\|Q96SK2\|TM209_HUMAN | TMEM209 | 62.88 | 3.6406 |
| 4 | 4 | sp\|Q9NX63\|MIC19_HUMAN | CHCHD3 | 26.14 | 3.628 |
| 4 | 4 | sp\|P26368\|U2AF2_HUMAN | U2AF2 | 53.47 | 3.6016 |
| 4 | 4 | sp\|A6NJ78\|MET15_HUMAN | METTL15 | 46.09 | 3.5927 |
| 4 | 4 | sp\|O75400\|PR40A_HUMAN | PRPF40A | 108.74 | 3.5794 |
| 4 | 4 | sp\|O15269\|SPTC1_HUMAN | SPTLC1 | 52.71 | 3.5413 |
| 4 | 4 | sp\|O43615\|TIM44_HUMAN | TIMM44 | 51.32 | 3.5271 |
| 4 | 4 | sp\|Q9Y5B9\|SP16H_HUMAN | SUPT16H | 119.84 | 3.4159 |
| 4 | 4 | sp\|Q14974\|IMB1_HUMAN | KPNB1 | 97.11 | 3.3547 |
| 4 | 4 | sp\|O60762\|DPM1_HUMAN | DPM1 | 29.62 | 3.2916 |
| 4 | 4 | sp\|Q9NRK6\|ABCBA_HUMAN | ABCB10 | 79.1 | 3.2617 |
| 4 | 4 | sp\|Q01831\|XPC_HUMAN | XPC | 105.89 | 3.2417 |
| 4 | 4 | sp\|Q9BSD7\|NTPCR_HUMAN | NTPCR | 20.7 | 3.2165 |
| 4 | 4 | sp\|Q9P0J0\|NDUAD_HUMAN | NDUFA13 | 16.69 | 3.1941 |
| 4 | 4 | sp\|Q9UJZ1\|STML2_HUMAN | STOML2 | 38.51 | 3.13 |
| 4 | 4 | sp\|Q13601\|KRR1_HUMAN | KRR1 | 43.64 | 3.1253 |
| 4 | 4 | sp\|P04843\|RPN1_HUMAN | RPN1 | 68.53 | 3.0947 |
| 4 | 4 | sp\|Q9BPW8\|NIPS1_HUMAN | NIPSNAP1 | 33.29 | 3.0705 |
| 4 | 4 | sp\|Q9H9P8\|L2HDH_HUMAN | L2HGDH | 50.28 | 3.0617 |
| 4 | 4 | sp\|P17844\|DDX5_HUMAN | DDX5 | 69.1 | 3.0394 |
| 4 | 4 | sp\|O43809\|CPSF5_HUMAN | NUDT21 | 26.21 | 3.0246 |
| 4 | 4 | sp\|P26641\|EF1G_HUMAN | EEF1G | 50.09 | 3.0126 |
| 4 | 4 | sp\|Q96HW7\|INT4_HUMAN | INTS4 | 108.1 | 3.0126 |
| 4 | 4 | sp\|Q8WYP5\|ELYS_HUMAN | AHCTF1 | 252.34 | 2.9942 |
| 4 | 4 | sp\|O43837\|IDH3B_HUMAN | IDH3B | 42.16 | 2.9905 |
| 4 | 4 | sp\|Q03701\|CEBPZ_HUMAN | CEBPZ | 120.9 | 2.9709 |
| 4 | 4 | sp\|Q14966\|ZN638_HUMAN | ZNF638 | 220.49 | 2.9484 |
| 4 | 4 | sp\|P00367\|DHE3_HUMAN | GLUD1 | 61.36 | 2.9254 |
| 4 | 4 | sp\|O76031\|CLPX_HUMAN | CLPX | 69.18 | 2.9124 |
| 4 | 4 | sp\|Q9UDR5\|AASS_HUMAN | AASS | 102.07 | 2.8915 |
| 4 | 4 | sp\|Q9BVA1\|TBB2B_HUMAN | TUBB2B | 49.92 | 2.8539 |
| 4 | 4 | sp\|Q9NXF1\|TEX10_HUMAN | TEX10 | 105.61 | 2.846 |
| 4 | 4 | sp\|Q9H936\|GHC1_HUMAN | SLC25A22 | 34.45 | 2.8371 |
| 4 | 4 | sp\|P08559\|ODPA_HUMAN | PDHA1 | 43.27 | 2.8323 |
| 4 | 4 | sp\|P46821\|MAP1B_HUMAN | MAP1B | 270.47 | 2.6704 |
| 4 | 4 | sp\|Q96PK6\|RBM14_HUMAN | RBM14 | 69.45 | 2.6673 |
| 4 | 4 | sp\|Q9NR30\|DDX21_HUMAN | DDX21 | 87.29 | 2.5986 |
| 4 | 4 | sp\|Q9NRG9\|AAAS_HUMAN | AAAS | 59.54 | 2.5796 |
| 4 | 4 | sp\|A3KMH1\|VWA8_HUMAN | VWA8 | 214.69 | 2.2576 |
| 4 | 4 | sp\|Q86UT6\|NLRX1_HUMAN | NLRX1 | 107.55 | 2.2278 |
| 4 | 4 | sp\|Q9ULK5\|VANG2_HUMAN | VANGL2 | 59.68 | 2.1911 |
| 3 | 4 | sp\|P48735\|IDHP_HUMAN | IDH2 | 50.88 | 3.7507 |
| 3 | 4 | sp\|P13804\|ETFA_HUMAN | ETFA | 35.06 | 3.5786 |
| 3 | 4 | sp\|Q9H2S9\|IKZF4_HUMAN | IKZF4 | 64.07 | 3.5119 |
| 3 | 4 | sp\|Q9H7H0\|MET17_HUMAN | METTL17 | 50.7 | 3.5054 |
| 3 | 4 | sp\|Q12905\|ILF2_HUMAN | ILF2 | 43.04 | 3.3415 |
| 3 | 4 | sp\|Q96EP5\|DAZP1_HUMAN | DAZAP1 | 43.36 | 3.3015 |
| 2 | 4 | sp\|Q93079\|H2B1H_HUMAN | HIST1H2BH | 13.88 | 2.1574 |
| 3 | 3 | sp\|Q00839\|HNRPU_HUMAN | HNRNPU | 90.53 | 4.8236 |
| 3 | 3 | sp\|Q5UIP0\|RIF1_HUMAN | RIF1 | 274.29 | 4.3695 |
| 3 | 3 | sp\|Q9Y5M8\|SRPRB_HUMAN | SRPRB | 29.68 | 4.3599 |
| 3 | 3 | sp\|Q96BW9\|TAM41_HUMAN | TAMM41 | 51.03 | 4.3564 |
| 3 | 3 | sp\|Q8NI60\|COQ8A_HUMAN | COQ8A | 71.9 | 4.3561 |
| 3 | 3 | sp\|P08195\|4F2_HUMAN | SLC3A2 | 67.95 | 4.1193 |
| 3 | 3 | sp\|O94813\|SLIT2_HUMAN | SLIT2 | 169.76 | 4.0788 |
| 3 | 3 | sp\|Q9UQE7\|SMC3_HUMAN | SMC3 | 141.45 | 4.0404 |
| 3 | 3 | sp\|Q6NSZ9\|ZSC25_HUMAN | ZSCAN25 | 61.44 | 4.0337 |
| 3 | 3 | sp\|P51648\|AL3A2_HUMAN | ALDH3A2 | 54.81 | 4.0321 |
| 3 | 3 | sp\|Q6IAN0\|DRS7B_HUMAN | DHRS7B | 35.1 | 4.0295 |
| 3 | 3 | sp\|Q14498\|RBM39_HUMAN | RBM39 | 59.34 | 3.9624 |
| 3 | 3 | sp\|P23634\|AT2B4_HUMAN | ATP2B4 | 137.83 | 3.9226 |
| 3 | 3 | sp\|Q13435\|SF3B2_HUMAN | SF3B2 | 100.16 | 3.8939 |
| 3 | 3 | sp\|Q07021\|C1QBP_HUMAN | C1QBP | 31.34 | 3.806 |
| 3 | 3 | sp\|O75616\|ERAL1_HUMAN | ERAL1 | 48.32 | 3.7878 |
| 3 | 3 | sp\|A8CG34\|P121C_HUMAN | POM121C | 124.98 | 3.7762 |
| 3 | 3 | sp\|O94906\|PRP6_HUMAN | PRPF6 | 106.86 | 3.7503 |
| 3 | 3 | sp\|P53597\|SUCA_HUMAN | SUCLG1 | 36.23 | 3.721 |
| 3 | 3 | sp\|Q86Y07\|VRK2_HUMAN | VRK2 | 58.1 | 3.697 |
| 3 | 3 | sp\|Q5JTV8\|TOIP1_HUMAN | TOR1AIP1 | 66.21 | 3.6383 |
| 3 | 3 | sp\|P32189\|GLPK_HUMAN | GK | 61.21 | 3.5989 |
| 3 | 3 | sp\|P51571\|SSRD_HUMAN | SSR4 | 18.99 | 3.5681 |
| 3 | 3 | sp\|Q96D53\|COQ8B_HUMAN | COQ8B | 60.03 | 3.559 |
| 3 | 3 | sp\|P02545\|LMNA_HUMAN | LMNA | 74.09 | 3.5564 |
| 3 | 3 | tr\|F8VXC8\|F8VXC8_HUMAN | SMARCC2 | 136.1 | 3.4843 |
| 3 | 3 | tr\|Q3B7X4\|Q3B7X4_HUMAN | IMMT | 40.47 | 3.4796 |
| 3 | 3 | sp\|Q9Y679\|AUP1_HUMAN | AUP1 | 52.99 | 3.3718 |
| 3 | 3 | sp\|P13010\|XRCC5_HUMAN | XRCC5 | 82.65 | 3.3491 |
| 3 | 3 | sp\|P32322\|P5CR1_HUMAN | PYCR1 | 33.34 | 3.3089 |
| 3 | 3 | sp\|Q9UMS4\|PRP19_HUMAN | PRPF19 | 55.15 | 3.2974 |
| 3 | 3 | sp\|P12956\|XRCC6_HUMAN | XRCC6 | 69.8 | 3.2967 |
| 3 | 3 | sp\|P49755\|TMEDA_HUMAN | TMED10 | 24.96 | 3.2947 |
| 3 | 3 | sp\|Q9Y678\|COPG1_HUMAN | COPG1 | 97.66 | 3.2391 |
| 3 | 3 | sp\|P43307\|SSRA_HUMAN | SSR1 | 32.22 | 3.212 |
| 3 | 3 | sp\|O75592\|MYCB2_HUMAN | MYCBP2 | 509.76 | 3.1986 |
| 3 | 3 | sp\|Q14103\|HNRPD_HUMAN | HNRNPD | 38.41 | 3.1817 |
| 3 | 3 | sp\|Q9BX10\|GTPB2_HUMAN | GTPBP2 | 65.73 | 3.1504 |
| 3 | 3 | sp\|O95299\|NDUAA_HUMAN | NDUFA10 | 40.72 | 3.1004 |
| 3 | 3 | sp\|P49792\|RBP2_HUMAN | RANBP2 | 357.97 | 3.071 |
| 3 | 3 | sp\|P28288\|ABCD3_HUMAN | ABCD3 | 75.43 | 3.0169 |
| 3 | 3 | sp\|Q92947\|GCDH_HUMAN | GCDH | 48.1 | 3.0023 |
| 3 | 3 | sp\|Q9BW92\|SYTM_HUMAN | TARS2 | 80.99 | 2.9891 |
| 3 | 3 | sp\|O43824\|GTPB6_HUMAN | GTPBP6 | 56.85 | 2.9889 |
| 3 | 3 | sp\|Q9H583\|HEAT1_HUMAN | HEATR1 | 242.22 | 2.9281 |
| 3 | 3 | sp\|Q9H0U3\|MAGT1_HUMAN | MAGT1 | 38.01 | 2.916 |
| 3 | 3 | sp\|Q8IXB1\|DJC10_HUMAN | DNAJC10 | 91.02 | 2.9101 |
| 3 | 3 | sp\|P57088\|TMM33_HUMAN | TMEM33 | 27.96 | 2.8935 |
| 3 | 3 | sp\|P20020\|AT2B1_HUMAN | ATP2B1 | 138.67 | 2.8139 |
| 3 | 3 | sp\|Q9H857\|NT5D2_HUMAN | NT5DC2 | 60.68 | 2.811 |
| 3 | 3 | sp\|P54136\|SYRC_HUMAN | RARS | 75.33 | 2.791 |
| 3 | 3 | sp\|P40937\|RFC5_HUMAN | RFC5 | 38.47 | 2.7891 |
| 3 | 3 | sp\|Q9BTX1\|NDC1_HUMAN | NDC1 | 76.26 | 2.7416 |
| 3 | 3 | sp\|Q5T160\|SYRM_HUMAN | RARS2 | 65.46 | 2.7067 |
| 3 | 3 | sp\|Q9Y4W2\|LAS1L_HUMAN | LAS1L | 83.01 | 2.6959 |
| 3 | 3 | sp\|Q9NZ01\|TECR_HUMAN | TECR | 36.01 | 2.6888 |
| 3 | 3 | sp\|Q8WWC4\|MAIP1_HUMAN | MAIP1 | 32.52 | 2.6849 |
| 3 | 3 | sp\|Q9UL03\|INT6_HUMAN | INTS6 | 100.33 | 2.6767 |
| 3 | 3 | sp\|Q13123\|RED_HUMAN | IK | 65.56 | 2.6747 |
| 3 | 3 | sp\|O60318\|GANP_HUMAN | MCM3AP | 218.27 | 2.6195 |
| 3 | 3 | sp\|Q9HBE1\|PATZ1_HUMAN | PATZ1 | 74.01 | 2.6111 |
| 3 | 3 | sp\|O94874\|UFL1_HUMAN | UFL1 | 89.54 | 2.5668 |
| 3 | 3 | sp\|Q96HS1\|PGAM5_HUMAN | PGAM5 | 31.98 | 2.5394 |
| 3 | 3 | sp\|O43143\|DHX15_HUMAN | DHX15 | 90.88 | 2.4658 |
| 3 | 3 | sp\|P63092\|GNAS2_HUMAN | GNAS | 45.64 | 2.3729 |
| 3 | 3 | sp\|Q9HC21\|TPC_HUMAN | SLC25A19 | 35.49 | 2.0792 |
| 2 | 3 | sp\|Q01650\|LAT1_HUMAN | SLC7A5 | 54.97 | 4.3582 |
| 2 | 3 | sp\|O95674\|CDS2_HUMAN | CDS2 | 51.38 | 4.0086 |
| 2 | 3 | sp\|O75251\|NDUS7_HUMAN | NDUFS7 | 23.55 | 3.655 |
| 2 | 3 | sp\|Q9UBM7\|DHCR7_HUMAN | DHCR7 | 54.45 | 3.0868 |
| 2 | 3 | sp\|Q9Y277\|VDAC3_HUMAN | VDAC3 | 30.64 | 3.0513 |
| 2 | 3 | sp\|P04792\|HSPB1_HUMAN | HSPB1 | 22.77 | 2.9885 |
| 2 | 3 | sp\|O14925\|TIM23_HUMAN | TIMM23 | 21.93 | 2.985 |
| 2 | 3 | sp\|P14618\|KPYM_HUMAN | PKM | 57.9 | 2.9626 |
| 2 | 3 | sp\|Q9UM00\|TMCO1_HUMAN | TMCO1 | 21.16 | 2.6723 |
| 2 | 3 | sp\|Q92782\|DPF1_HUMAN | DPF1 | 42.47 | 1.8419 |
| 2 | 2 | sp\|Q9NS69\|TOM22_HUMAN | TOMM22 | 15.51 | 5.021 |
| 2 | 2 | sp\|Q99805\|TM9S2_HUMAN | TM9SF2 | 75.73 | 4.898 |
| 2 | 2 | sp\|A1L0T0\|ILVBL_HUMAN | ILVBL | 67.82 | 4.8795 |
| 2 | 2 | sp\|Q9BSF4\|TIM29_HUMAN | TIMM29 | 29.22 | 4.7691 |
| 2 | 2 | sp\|Q9NNW5\|WDR6_HUMAN | WDR6 | 121.65 | 4.6122 |
| 2 | 2 | sp\|Q70CQ3\|UBP30_HUMAN | USP30 | 58.47 | 4.5749 |
| 2 | 2 | sp\|Q9GZR7\|DDX24_HUMAN | DDX24 | 96.27 | 4.5606 |
| 2 | 2 | sp\|P09622\|DLDH_HUMAN | DLD | 54.14 | 4.506 |
| 2 | 2 | sp\|P00403\|COX2_HUMAN | MT-CO2 | 25.55 | 4.3826 |
| 2 | 2 | sp\|Q8TB37\|NUBPL_HUMAN | NUBPL | 34.06 | 4.2762 |
| 2 | 2 | sp\|Q5JPH6\|SYEM_HUMAN | EARS2 | 58.65 | 4.2723 |
| 2 | 2 | sp\|P07910\|HNRPC_HUMAN | HNRNPC | 33.65 | 4.2686 |
| 2 | 2 | sp\|P47985\|UCRI_HUMAN | UQCRFS1 | 29.65 | 4.2492 |
| 2 | 2 | sp\|Q9NPI1\|BRD7_HUMAN | BRD7 | 74.09 | 4.2027 |
| 2 | 2 | sp\|P41208\|CETN2_HUMAN | CETN2 | 19.73 | 4.1872 |
| 2 | 2 | sp\|O00165\|HAX1_HUMAN | HAX1 | 31.6 | 4.1829 |
| 2 | 2 | sp\|P55265\|DSRAD_HUMAN | ADAR | 135.98 | 4.1552 |
| 2 | 2 | sp\|Q9NUQ2\|PLCE_HUMAN | AGPAT5 | 42.04 | 4.1535 |
| 2 | 2 | sp\|Q68CQ7\|GL8D1_HUMAN | GLT8D1 | 41.91 | 4.0458 |
| 2 | 2 | sp\|Q5HYI7\|MTX3_HUMAN | MTX3 | 35.07 | 4.0096 |
| 2 | 2 | sp\|P07437\|TBB5_HUMAN | TUBB | 49.64 | 3.9765 |
| 2 | 2 | sp\|P13674\|P4HA1_HUMAN | P4HA1 | 61.01 | 3.9399 |
| 2 | 2 | sp\|Q8WY36\|BBX_HUMAN | BBX | 105.06 | 3.912 |
| 2 | 2 | sp\|Q6DD88\|ATLA3_HUMAN | ATL3 | 60.5 | 3.8159 |
| 2 | 2 | sp\|O00116\|ADAS_HUMAN | AGPS | 72.87 | 3.8089 |
| 2 | 2 | sp\|P03886\|NU1M_HUMAN | MT-ND1 | 35.64 | 3.785 |
| 2 | 2 | sp\|O15270\|SPTC2_HUMAN | SPTLC2 | 62.88 | 3.7695 |
| 2 | 2 | sp\|Q9UDX5\|MTFP1_HUMAN | MTFP1 | 18 | 3.7587 |
| 2 | 2 | sp\|P52292\|IMA1_HUMAN | KPNA2 | 57.83 | 3.7421 |
| 2 | 2 | sp\|O95639\|CPSF4_HUMAN | CPSF4 | 30.23 | 3.7332 |
| 2 | 2 | sp\|Q96C36\|P5CR2_HUMAN | PYCR2 | 33.62 | 3.6947 |
| 2 | 2 | sp\|P11498\|PYC_HUMAN | PC | 129.55 | 3.6878 |
| 2 | 2 | sp\|O00410\|IP05_HUMAN | IPO5 | 123.55 | 3.6863 |
| 2 | 2 | sp\|O14654\|IRS4_HUMAN | IRS4 | 133.68 | 3.685 |
| 2 | 2 | sp\|Q9NVH1\|DJC11_HUMAN | DNAJC11 | 63.24 | 3.6387 |
| 2 | 2 | sp\|Q86VP6\|CAND1_HUMAN | CAND1 | 136.29 | 3.6258 |
| 2 | 2 | sp\|P05412\|JUN_HUMAN | JUN | 35.65 | 3.6102 |
| 2 | 2 | sp\|Q00587\|BORG5_HUMAN | CDC42EP1 | 40.27 | 3.6034 |
| 2 | 2 | sp\|P62987\|RL40_HUMAN | UBA52 | 14.72 | 3.5869 |
| 2 | 2 | sp\|P12004\|PCNA_HUMAN | PCNA | 28.75 | 3.5839 |
| 2 | 2 | sp\|Q9NRZ9\|HELLS_HUMAN | HELLS | 97.01 | 3.5446 |
| 2 | 2 | sp\|Q15393\|SF3B3_HUMAN | SF3B3 | 135.49 | 3.5349 |
| 2 | 2 | sp\|Q9NVI1\|FANCI_HUMAN | FANCI | 149.23 | 3.532 |
| 2 | 2 | sp\|P33527\|MRP1_HUMAN | ABCC1 | 171.48 | 3.5224 |
| 2 | 2 | sp\|Q03252\|LMNB2_HUMAN | LMNB2 | 69.91 | 3.5134 |
| 2 | 2 | sp\|O00400\|ACATN_HUMAN | SLC33A1 | 60.87 | 3.5111 |
| 2 | 2 | sp\|Q92522\|H1X_HUMAN | H1FX | 22.47 | 3.4867 |
| 2 | 2 | sp\|Q9UBU9\|NXF1_HUMAN | NXF1 | 70.14 | 3.471 |
| 2 | 2 | sp\|Q15365\|PCBP1_HUMAN | PCBP1 | 37.47 | 3.4358 |
| 2 | 2 | sp\|Q9H061\|T126A_HUMAN | TMEM126A | 21.51 | 3.4112 |
| 2 | 2 | sp\|P52429\|DGKE_HUMAN | DGKE | 63.88 | 3.3745 |
| 2 | 2 | sp\|O00257\|CBX4_HUMAN | CBX4 | 61.33 | 3.344 |
| 2 | 2 | sp\|P54886\|P5CS_HUMAN | ALDH18A1 | 87.25 | 3.3363 |
| 2 | 2 | sp\|O43491\|E41L2_HUMAN | EPB41L2 | 112.52 | 3.3288 |
| 2 | 2 | sp\|Q5JTZ9\|SYAM_HUMAN | AARS2 | 107.27 | 3.3224 |
| 2 | 2 | sp\|P08574\|CY1_HUMAN | CYC1 | 35.4 | 3.3097 |
| 2 | 2 | sp\|P48651\|PTSS1_HUMAN | PTDSS1 | 55.49 | 3.2672 |
| 2 | 2 | sp\|Q53R41\|FAKD1_HUMAN | FASTKD1 | 97.35 | 3.263 |
| 2 | 2 | sp\|Q92542\|NICA_HUMAN | NCSTN | 78.36 | 3.2618 |
| 2 | 2 | sp\|Q8IY17\|PLPL6_HUMAN | PNPLA6 | 149.9 | 3.2524 |
| 2 | 2 | sp\|P30825\|CTR1_HUMAN | SLC7A1 | 67.59 | 3.2044 |
| 2 | 2 | sp\|O15321\|TM9S1_HUMAN | TM9SF1 | 68.82 | 3.1856 |
| 2 | 2 | sp\|P31689\|DNJA1_HUMAN | DNAJA1 | 44.84 | 3.1792 |
| 2 | 2 | sp\|Q5JVF3\|PCID2_HUMAN | PCID2 | 46 | 3.1732 |
| 2 | 2 | sp\|Q8WUK0\|PTPM1_HUMAN | PTPMT1 | 22.83 | 3.1618 |
| 2 | 2 | sp\|O75396\|SC22B_HUMAN | SEC22B | 24.58 | 3.154 |
| 2 | 2 | sp\|Q9Y2J2\|E41L3_HUMAN | EPB41L3 | 120.6 | 3.1507 |
| 2 | 2 | sp\|O95573\|ACSL3_HUMAN | ACSL3 | 80.37 | 3.1429 |
| 2 | 2 | sp\|Q96N66\|MBOA7_HUMAN | MBOAT7 | 52.73 | 3.0857 |
| 2 | 2 | sp\|Q9UG63\|ABCF2_HUMAN | ABCF2 | 71.24 | 3.0777 |
| 2 | 2 | sp\|Q10469\|MGAT2_HUMAN | MGAT2 | 51.52 | 3.06 |
| 2 | 2 | sp\|Q68CP9\|ARID2_HUMAN | ARID2 | 197.27 | 3.0415 |
| 2 | 2 | sp\|Q8NC56\|LEMD2_HUMAN | LEMD2 | 56.94 | 3.0367 |
| 2 | 2 | sp\|Q9UBD5\|ORC3_HUMAN | ORC3 | 82.2 | 3.0212 |
| 2 | 2 | sp\|O00159\|MYO1C_HUMAN | MYO1C | 121.61 | 3.0205 |
| 2 | 2 | sp\|P56134\|ATPK_HUMAN | ATP5J2 | 10.91 | 3.0031 |
| 2 | 2 | sp\|P38117\|ETFB_HUMAN | ETFB | 27.83 | 3.001 |
| 2 | 2 | sp\|P12235\|ADT1_HUMAN | SLC25A4 | 33.04 | 2.929 |
| 2 | 2 | sp\|Q5C9Z4\|NOM1_HUMAN | NOM1 | 96.2 | 2.8956 |
| 2 | 2 | sp\|Q8TCT9\|HM13_HUMAN | HM13 | 41.46 | 2.8954 |
| 2 | 2 | sp\|Q9H2D1\|MFTC_HUMAN | SLC25A32 | 35.38 | 2.892 |
| 2 | 2 | sp\|P17987\|TCPA_HUMAN | TCP1 | 60.31 | 2.8893 |
| 2 | 2 | sp\|Q9BYN8\|RT26_HUMAN | MRPS26 | 24.2 | 2.8798 |
| 2 | 2 | sp\|Q13428\|TCOF_HUMAN | TCOF1 | 152.02 | 2.8687 |
| 2 | 2 | sp\|Q8N442\|GUF1_HUMAN | GUF1 | 74.28 | 2.8578 |
| 2 | 2 | sp\|Q9NSI2\|F207A_HUMAN | FAM207A | 25.44 | 2.8529 |
| 2 | 2 | sp\|O75323\|NIPS2_HUMAN | NIPSNAP2 | 33.72 | 2.8489 |
| 2 | 2 | sp\|Q8NF37\|PCAT1_HUMAN | LPCAT1 | 59.11 | 2.8362 |
| 2 | 2 | sp\|P46087\|NOP2_HUMAN | NOP2 | 89.25 | 2.83 |
| 2 | 2 | sp\|Q15120\|PDK3_HUMAN | PDK3 | 46.91 | 2.8249 |
| 2 | 2 | sp\|Q9UGN5\|PARP2_HUMAN | PARP2 | 66.16 | 2.8221 |
| 2 | 2 | sp\|Q12788\|TBL3_HUMAN | TBL3 | 88.98 | 2.8072 |
| 2 | 2 | sp\|Q13505\|MTX1_HUMAN | MTX1 | 51.44 | 2.802 |
| 2 | 2 | sp\|Q9H0A0\|NAT10_HUMAN | NAT10 | 115.66 | 2.785 |
| 2 | 2 | sp\|Q5VV42\|CDKAL_HUMAN | CDKAL1 | 65.07 | 2.7816 |
| 2 | 2 | sp\|Q9H223\|EHD4_HUMAN | EHD4 | 61.14 | 2.7606 |
| 2 | 2 | sp\|P38435\|VKGC_HUMAN | GGCX | 87.5 | 2.7259 |
| 2 | 2 | sp\|Q99459\|CDC5L_HUMAN | CDC5L | 92.19 | 2.7222 |
| 2 | 2 | sp\|Q99653\|CHP1_HUMAN | CHP1 | 22.44 | 2.7104 |
| 2 | 2 | sp\|Q9HD45\|TM9S3_HUMAN | TM9SF3 | 67.84 | 2.6916 |
| 2 | 2 | sp\|Q9Y4A5\|TRRAP_HUMAN | TRRAP | 437.32 | 2.6864 |
| 2 | 2 | sp\|Q9Y6J9\|TAF6L_HUMAN | TAF6L | 67.77 | 2.6785 |
| 2 | 2 | sp\|O95347\|SMC2_HUMAN | SMC2 | 135.57 | 2.6624 |
| 2 | 2 | sp\|Q92544\|TM9S4_HUMAN | TM9SF4 | 74.47 | 2.6428 |
| 2 | 2 | sp\|P11387\|TOP1_HUMAN | TOP1 | 90.67 | 2.6258 |
| 2 | 2 | sp\|Q9P032\|NDUF4_HUMAN | NDUFAF4 | 20.25 | 2.619 |
| 2 | 2 | sp\|Q12906\|ILF3_HUMAN | ILF3 | 95.28 | 2.6162 |
| 2 | 2 | sp\|Q9BXW7\|HDHD5_HUMAN | HDHD5 | 46.29 | 2.6115 |
| 2 | 2 | sp\|P49821\|NDUV1_HUMAN | NDUFV1 | 50.78 | 2.5884 |
| 2 | 2 | sp\|O00192\|ARVC_HUMAN | ARVCF | 104.58 | 2.521 |
| 2 | 2 | sp\|Q9BVK6\|TMED9_HUMAN | TMED9 | 27.26 | 2.5109 |
| 2 | 2 | sp\|Q6ZXV5\|TMTC3_HUMAN | TMTC3 | 103.94 | 2.5017 |
| 2 | 2 | sp\|O76062\|ERG24_HUMAN | TM7SF2 | 46.38 | 2.4483 |
| 2 | 2 | sp\|Q9Y512\|SAM50_HUMAN | SAMM50 | 51.94 | 2.4034 |
| 2 | 2 | sp\|Q5TA45\|INT11_HUMAN | INTS11 | 67.62 | 2.4003 |
| 2 | 2 | sp\|P28370\|SMCA1_HUMAN | SMARCA1 | 122.53 | 2.3685 |
| 2 | 2 | sp\|P17480\|UBF1_HUMAN | UBTF | 89.35 | 2.3649 |
| 2 | 2 | sp\|P84103\|SRSF3_HUMAN | SRSF3 | 19.32 | 2.2759 |
| 2 | 2 | sp\|Q14527\|HLTF_HUMAN | HLTF | 113.86 | 2.2533 |
| 2 | 2 | sp\|P61619\|S61A1_HUMAN | SEC61A1 | 52.23 | 2.2427 |
| 2 | 2 | sp\|Q9Y2R9\|RT07_HUMAN | MRPS7 | 28.12 | 2.1584 |
| 2 | 2 | sp\|Q9BSJ2\|GCP2_HUMAN | TUBGCP2 | 102.47 | 1.8316 |
| 1 | 2 | sp\|Q01130\|SRSF2_HUMAN | SRSF2 | 25.46 | 4.3513 |
| 1 | 2 | sp\|P16104\|H2AX_HUMAN | H2AFX | 15.14 | 3.4679 |
| 1 | 2 | sp\|O94805\|ACL6B_HUMAN | ACTL6B | 46.85 | 3.0937 |
| 1 | 2 | sp\|Q99959\|PKP2_HUMAN | PKP2 | 97.35 | 3.0573 |
| 1 | 2 | sp\|Q9BYX7\|ACTBM_HUMAN | POTEKP | 41.99 | 3.0304 |
| 1 | 2 | sp\|Q9Y3E0\|GOT1B_HUMAN | GOLT1B | 15.42 | 2.5183 |
| 1 | 2 | sp\|Q9Y2X0\|MED16_HUMAN | MED16 | 96.73 | 2.3369 |
| 1 | 2 | sp\|Q9P104\|DOK5_HUMAN | DOK5 | 35.44 | 2.0759 |
| 1 | 1 | sp\|Q9NPL8\|TIDC1_HUMAN | TIMMDC1 | 32.16 | 5.5872 |
| 1 | 1 | sp\|Q75QN2\|INT8_HUMAN | INTS8 | 113.02 | 5.1795 |
| 1 | 1 | sp\|Q7L8L6\|FAKD5_HUMAN | FASTKD5 | 86.52 | 5.1458 |
| 1 | 1 | sp\|Q15287\|RNPS1_HUMAN | RNPS1 | 34.19 | 4.9637 |
| 1 | 1 | sp\|Q8WUA4\|TF3C2_HUMAN | GTF3C2 | 100.62 | 4.8457 |
| 1 | 1 | sp\|Q9Y2Q3\|GSTK1_HUMAN | GSTK1 | 25.48 | 4.8083 |
| 1 | 1 | sp\|Q9H845\|ACAD9_HUMAN | ACAD9 | 68.72 | 4.7486 |
| 1 | 1 | sp\|P61978\|HNRPK_HUMAN | HNRNPK | 50.94 | 4.7158 |
| 1 | 1 | sp\|O94766\|B3GA3_HUMAN | B3GAT3 | 37.1 | 4.7011 |
| 1 | 1 | sp\|O15554\|KCNN4_HUMAN | KCNN4 | 47.66 | 4.684 |
| 1 | 1 | sp\|O60884\|DNJA2_HUMAN | DNAJA2 | 45.72 | 4.6496 |
| 1 | 1 | sp\|P62995\|TRA2B_HUMAN | TRA2B | 33.65 | 4.633 |
| 1 | 1 | sp\|Q8N6R0\|MET13_HUMAN | METTL13 | 78.72 | 4.5641 |
| 1 | 1 | tr\|B7ZAF6\|B7ZAF6_HUMAN | SUCLA2 | 43.83 | 4.5502 |
| 1 | 1 | sp\|Q04837\|SSBP_HUMAN | SSBP1 | 17.25 | 4.5487 |
| 1 | 1 | sp\|Q6B0I6\|KDM4D_HUMAN | KDM4D | 58.57 | 4.5398 |
| 1 | 1 | sp\|P56962\|STX17_HUMAN | STX17 | 33.38 | 4.5351 |
| 1 | 1 | sp\|Q9Y3D7\|TIM16_HUMAN | PAM16 | 13.82 | 4.4422 |
| 1 | 1 | sp\|P08708\|RS17_HUMAN | RPS17 | 15.54 | 4.4124 |
| 1 | 1 | sp\|Q9BT22\|ALG1_HUMAN | ALG1 | 52.48 | 4.402 |
| 1 | 1 | tr\|Q2M1J6\|Q2M1J6_HUMAN | OXA1L | 55.35 | 4.3966 |
| 1 | 1 | sp\|O75528\|TADA3_HUMAN | TADA3 | 48.87 | 4.3954 |
| 1 | 1 | sp\|Q9UNQ2\|DIM1_HUMAN | DIMT1 | 35.21 | 4.3536 |
| 1 | 1 | sp\|O95470\|SGPL1_HUMAN | SGPL1 | 63.48 | 4.3015 |
| 1 | 1 | sp\|Q86Y91\|KI18B_HUMAN | KIF18B | 94.16 | 4.2375 |
| 1 | 1 | sp\|Q9NWW5\|CLN6_HUMAN | CLN6 | 35.9 | 4.2186 |
| 1 | 1 | sp\|Q969Y2\|GTPB3_HUMAN | GTPBP3 | 52.03 | 4.1992 |
| 1 | 1 | sp\|O43159\|RRP8_HUMAN | RRP8 | 50.68 | 4.1825 |
| 1 | 1 | sp\|Q8N6L1\|KTAP2_HUMAN | KRTCAP2 | 14.67 | 4.1782 |
| 1 | 1 | sp\|O75531\|BAF_HUMAN | BANF1 | 10.05 | 4.1148 |
| 1 | 1 | sp\|O94887\|FARP2_HUMAN | FARP2 | 119.81 | 4.1008 |
| 1 | 1 | sp\|Q96GC9\|VMP1_HUMAN | VMP1 | 46.21 | 4.0888 |
| 1 | 1 | sp\|Q9BPX6\|MICU1_HUMAN | MICU1 | 54.32 | 4.0864 |
| 1 | 1 | sp\|O43347\|MSI1H_HUMAN | MSI1 | 39.1 | 4.0779 |
| 1 | 1 | tr\|B4DR34\|B4DR34_HUMAN | | 36.86 | 4.074 |
| 1 | 1 | sp\|Q8TAE8\|G45IP_HUMAN | GADD45GIP1 | 25.37 | 4.0691 |
| 1 | 1 | sp\|Q8N465\|D2HDH_HUMAN | D2HGDH | 56.38 | 4.0528 |
| 1 | 1 | sp\|O95140\|MFN2_HUMAN | MFN2 | 86.35 | 4.0323 |
| 1 | 1 | sp\|P00387\|NB5R3_HUMAN | CYB5R3 | 34.21 | 4.0153 |
| 1 | 1 | sp\|Q15334\|L2GL1_HUMAN | LLGL1 | 115.35 | 4.0019 |
| 1 | 1 | sp\|Q9UHX1\|PUF60_HUMAN | PUF60 | 59.84 | 3.985 |
| 1 | 1 | sp\|Q96DA6\|TIM14_HUMAN | DNAJC19 | 12.49 | 3.9537 |
| 1 | 1 | sp\|O43390\|HNRPR_HUMAN | HNRNPR | 70.9 | 3.9371 |
| 1 | 1 | sp\|P62820\|RAB1A_HUMAN | RAB1A | 22.66 | 3.9346 |
| 1 | 1 | sp\|Q9HDC5\|JPH1_HUMAN | JPH1 | 71.64 | 3.9134 |
| 1 | 1 | sp\|Q8TBP6\|S2540_HUMAN | SLC25A40 | 38.1 | 3.8986 |
| 1 | 1 | sp\|O75352\|MPU1_HUMAN | MPDU1 | 26.62 | 3.8941 |
| 1 | 1 | sp\|O75533\|SF3B1_HUMAN | SF3B1 | 145.74 | 3.8767 |
| 1 | 1 | sp\|P35558\|PCKGC_HUMAN | PCK1 | 69.15 | 3.8646 |
| 1 | 1 | sp\|Q15459\|SF3A1_HUMAN | SF3A1 | 88.83 | 3.8594 |
| 1 | 1 | sp\|O14880\|MGST3_HUMAN | MGST3 | 16.51 | 3.8593 |
| 1 | 1 | sp\|Q6NTF9\|RHBD2_HUMAN | RHBDD2 | 39.18 | 3.819 |
| 1 | 1 | sp\|Q15233\|NONO_HUMAN | NONO | 54.2 | 3.7878 |
| 1 | 1 | sp\|Q9H4L4\|SENP3_HUMAN | SENP3 | 64.97 | 3.785 |
| 1 | 1 | sp\|Q96QD8\|S38A2_HUMAN | SLC38A2 | 55.99 | 3.7721 |
| 1 | 1 | sp\|Q9H8H3\|MET7A_HUMAN | METTL7A | 28.3 | 3.7715 |
| 1 | 1 | sp\|Q9NVH0\|EXD2_HUMAN | EXD2 | 70.31 | 3.7582 |
| 1 | 1 | sp\|Q9H5Q4\|TFB2M_HUMAN | TFB2M | 45.32 | 3.7433 |
| 1 | 1 | sp\|Q8IWA4\|MFN1_HUMAN | MFN1 | 84.05 | 3.742 |
| 1 | 1 | tr\|J3KN66\|J3KN66_HUMAN | TOR1AIP1 | 67.78 | 3.734 |
| 1 | 1 | sp\|O00148\|DX39A_HUMAN | DDX39A | 49.1 | 3.7271 |
| 1 | 1 | sp\|O75976\|CBPD_HUMAN | CPD | 152.84 | 3.726 |
| 1 | 1 | sp\|Q3SXY8\|AR13B_HUMAN | ARL13B | 48.61 | 3.7066 |
| 1 | 1 | sp\|P35232\|PHB_HUMAN | PHB | 29.79 | 3.701 |
| 1 | 1 | sp\|Q5SY16\|NOL9_HUMAN | NOL9 | 79.27 | 3.6809 |
| 1 | 1 | sp\|Q9Y3A6\|TMED5_HUMAN | TMED5 | 25.99 | 3.655 |
| 1 | 1 | sp\|Q9BZE1\|RM37_HUMAN | MRPL37 | 48.09 | 3.6351 |
| 1 | 1 | sp\|Q8NBN7\|RDH13_HUMAN | RDH13 | 35.91 | 3.6132 |
| 1 | 1 | sp\|Q6NUN9\|ZN746_HUMAN | ZNF746 | 69.09 | 3.6064 |
| 1 | 1 | sp\|P41252\|SYIC_HUMAN | IARS | 144.41 | 3.601 |
| 1 | 1 | sp\|Q96BN2\|TADA1_HUMAN | TADA1 | 37.36 | 3.5969 |
| 1 | 1 | sp\|P54652\|HSP72_HUMAN | HSPA2 | 69.98 | 3.5946 |
| 1 | 1 | sp\|Q02338\|BDH_HUMAN | BDH1 | 38.13 | 3.5915 |
| 1 | 1 | tr\|A0A1B0GTJ8\|A0A1B0GTJ8_HUMAN | ARID1B | 163.28 | 3.5712 |
| 1 | 1 | sp\|Q9Y5Y0\|FLVC1_HUMAN | FLVCR1 | 59.82 | 3.5588 |
| 1 | 1 | sp\|Q9H0H0\|INT2_HUMAN | INTS2 | 134.24 | 3.534 |
| 1 | 1 | tr\|H7BXI1\|H7BXI1_HUMAN | ESYT2 | 97.95 | 3.5218 |
| 1 | 1 | sp\|O00571\|DDX3X_HUMAN | DDX3X | 73.2 | 3.5084 |
| 1 | 1 | sp\|Q6P9B9\|INT5_HUMAN | INTS5 | 107.93 | 3.4997 |
| 1 | 1 | sp\|Q969X6\|UTP4_HUMAN | UTP4 | 76.84 | 3.4982 |
| 1 | 1 | sp\|Q7Z5K2\|WAPL_HUMAN | WAPL | 132.86 | 3.4906 |
| 1 | 1 | sp\|Q8NI27\|THOC2_HUMAN | THOC2 | 182.66 | 3.4868 |
| 1 | 1 | sp\|Q6YN16\|HSDL2_HUMAN | HSDL2 | 45.37 | 3.4781 |
| 1 | 1 | sp\|P83731\|RL24_HUMAN | RPL24 | 17.77 | 3.4775 |
| 1 | 1 | sp\|Q5BKZ1\|ZN326_HUMAN | ZNF326 | 65.61 | 3.4679 |
| 1 | 1 | IGKC_MOUSE | | 11.77 | 3.4619 |
| 1 | 1 | sp\|Q5T0B9\|ZN362_HUMAN | ZNF362 | 45.79 | 3.4575 |
| 1 | 1 | sp\|Q16698\|DECR_HUMAN | DECR1 | 36.04 | 3.4391 |
| 1 | 1 | sp\|Q6PIW4\|FIGL1_HUMAN | FIGNL1 | 74.03 | 3.4381 |
| 1 | 1 | sp\|Q14517\|FAT1_HUMAN | FAT1 | 505.96 | 3.4316 |
| 1 | 1 | sp\|Q8TAA9\|VANG1_HUMAN | VANGL1 | 59.94 | 3.4174 |
| 1 | 1 | sp\|Q8NBI6\|XXLT1_HUMAN | XXYLT1 | 43.78 | 3.3993 |
| 1 | 1 | sp\|P61225\|RAP2B_HUMAN | RAP2B | 20.49 | 3.3924 |
| 1 | 1 | sp\|Q9P2E9\|RRBP1_HUMAN | RRBP1 | 152.38 | 3.3909 |
| 1 | 1 | sp\|O14776\|TCRG1_HUMAN | TCERG1 | 123.82 | 3.3829 |
| 1 | 1 | sp\|P08670\|VIME_HUMAN | VIM | 53.62 | 3.3766 |
| 1 | 1 | sp\|P68363\|TBA1B_HUMAN | TUBA1B | 50.12 | 3.3736 |
| 1 | 1 | sp\|P62316\|SMD2_HUMAN | SNRPD2 | 13.52 | 3.3706 |
| 1 | 1 | sp\|Q8WY07\|CTR3_HUMAN | SLC7A3 | 67.13 | 3.3446 |
| 1 | 1 | sp\|O95070\|YIF1A_HUMAN | YIF1A | 31.99 | 3.3423 |
| 1 | 1 | sp\|Q9Y289\|SC5A6_HUMAN | SLC5A6 | 68.6 | 3.3106 |
| 1 | 1 | sp\|P62701\|RS4X_HUMAN | RPS4X | 29.58 | 3.3014 |
| 1 | 1 | sp\|Q9BVJ6\|UT14A_HUMAN | UTP14A | 87.92 | 3.2855 |
| 1 | 1 | sp\|Q9Y3T9\|NOC2L_HUMAN | NOC2L | 84.87 | 3.2781 |
| 1 | 1 | sp\|P05067\|A4_HUMAN | APP | 86.89 | 3.2681 |
| 1 | 1 | sp\|P98194\|AT2C1_HUMAN | ATP2C1 | 100.51 | 3.2635 |
| 1 | 1 | sp\|Q06587\|RING1_HUMAN | RING1 | 42.4 | 3.2538 |
| 1 | 1 | sp\|Q13148\|TADBP_HUMAN | TARDBP | 44.71 | 3.2457 |
| 1 | 1 | tr\|Q59G16\|Q59G16_HUMAN | | 127.3 | 3.2436 |
| 1 | 1 | sp\|Q9UKU7\|ACAD8_HUMAN | ACAD8 | 45.04 | 3.2308 |
| 1 | 1 | sp\|P06493\|CDK1_HUMAN | CDK1 | 34.07 | 3.2145 |
| 1 | 1 | sp\|Q86VI3\|IQGA3_HUMAN | IQGAP3 | 184.58 | 3.2033 |
| 1 | 1 | sp\|Q71UI9\|H2AV_HUMAN | H2AFV | 13.5 | 3.2018 |
| 1 | 1 | sp\|Q9Y584\|TIM22_HUMAN | TIMM22 | 20.02 | 3.1815 |
| 1 | 1 | sp\|Q96AA3\|RFT1_HUMAN | RFT1 | 60.3 | 3.1606 |
| 1 | 1 | sp\|O60830\|TI17B_HUMAN | TIMM17B | 18.26 | 3.1592 |
| 1 | 1 | sp\|Q8TEM1\|PO210_HUMAN | NUP210 | 204.98 | 3.1521 |
| 1 | 1 | sp\|P62241\|RS8_HUMAN | RPS8 | 24.19 | 3.1446 |
| 1 | 1 | sp\|P04406\|G3P_HUMAN | GAPDH | 36.03 | 3.1344 |
| 1 | 1 | sp\|Q96DA2\|RB39B_HUMAN | RAB39B | 24.61 | 3.1279 |
| 1 | 1 | sp\|Q9ULH0\|KDIS_HUMAN | KIDINS220 | 196.42 | 3.1272 |
| 1 | 1 | sp\|O60264\|SMCA5_HUMAN | SMARCA5 | 121.83 | 3.1176 |
| 1 | 1 | sp\|Q15572\|TAF1C_HUMAN | TAF1C | 95.15 | 3.1073 |
| 1 | 1 | sp\|Q9Y6M5\|ZNT1_HUMAN | SLC30A1 | 55.26 | 3.1001 |
| 1 | 1 | sp\|P19404\|NDUV2_HUMAN | NDUFV2 | 27.37 | 3.0934 |
| 1 | 1 | sp\|Q9Y2W1\|TR150_HUMAN | THRAP3 | 108.6 | 3.092 |
| 1 | 1 | sp\|Q9H0U9\|TSYL1_HUMAN | TSPYL1 | 49.16 | 3.0855 |
| 1 | 1 | sp\|Q9Y256\|FACE2_HUMAN | RCE1 | 35.81 | 3.0848 |
| 1 | 1 | sp\|O43913\|ORC5_HUMAN | ORC5 | 50.25 | 3.0745 |
| 1 | 1 | sp\|O00483\|NDUA4_HUMAN | NDUFA4 | 9.36 | 3.0468 |
| 1 | 1 | sp\|Q15366\|PCBP2_HUMAN | PCBP2 | 38.56 | 3.0445 |
| 1 | 1 | sp\|P35659\|DEK_HUMAN | DEK | 42.65 | 3.0425 |
| 1 | 1 | sp\|P24468\|COT2_HUMAN | NR2F2 | 45.54 | 3.0302 |
| 1 | 1 | sp\|Q16563\|SYPL1_HUMAN | SYPL1 | 28.55 | 3.0247 |
| 1 | 1 | sp\|Q9H300\|PARL_HUMAN | PARL | 42.16 | 3.0049 |
| 1 | 1 | sp\|P05091\|ALDH2_HUMAN | ALDH2 | 56.35 | 2.975 |
| 1 | 1 | sp\|Q13415\|ORC1_HUMAN | ORC1 | 97.29 | 2.9662 |
| 1 | 1 | sp\|Q9Y5A9\|YTHD2_HUMAN | YTHDF2 | 62.3 | 2.954 |
| 1 | 1 | sp\|P68871\|HBB_HUMAN | HBB | 15.99 | 2.9512 |
| 1 | 1 | sp\|O14656\|TOR1A_HUMAN | TOR1A | 37.78 | 2.9413 |
| 1 | 1 | sp\|Q9UJ14\|GGT7_HUMAN | GGT7 | 70.42 | 2.9379 |
| 1 | 1 | sp\|O95168\|NDUB4_HUMAN | NDUFB4 | 15.2 | 2.9313 |
| 1 | 1 | sp\|Q15392\|DHC24_HUMAN | DHCR24 | 60.06 | 2.9241 |
| 1 | 1 | sp\|Q9ULK4\|MED23_HUMAN | MED23 | 156.37 | 2.9228 |
| 1 | 1 | sp\|Q09161\|NCBP1_HUMAN | NCBP1 | 91.78 | 2.9153 |
| 1 | 1 | sp\|Q9Y3Z3\|SAMH1_HUMAN | SAMHD1 | 72.15 | 2.9067 |
| 1 | 1 | tr\|B3KUE6\|B3KUE6_HUMAN | | 30.19 | 2.8998 |
| 1 | 1 | sp\|Q92643\|GPI8_HUMAN | PIGK | 45.22 | 2.8928 |
| 1 | 1 | sp\|Q9BUN8\|DERL1_HUMAN | DERL1 | 28.78 | 2.8902 |
| 1 | 1 | sp\|Q9BQ39\|DDX50_HUMAN | DDX50 | 82.51 | 2.889 |
| 1 | 1 | sp\|Q9HAV4\|XPO5_HUMAN | XPO5 | 136.22 | 2.8793 |
| 1 | 1 | sp\|O75964\|ATP5L_HUMAN | ATP5L | 11.42 | 2.8756 |
| 1 | 1 | sp\|Q9NTJ3\|SMC4_HUMAN | SMC4 | 147.09 | 2.8579 |
| 1 | 1 | sp\|Q14008\|CKAP5_HUMAN | CKAP5 | 225.35 | 2.8551 |
| 1 | 1 | sp\|Q8NCH0\|CHSTE_HUMAN | CHST14 | 42.97 | 2.8501 |
| 1 | 1 | sp\|Q92604\|LGAT1_HUMAN | LPGAT1 | 43.06 | 2.8286 |
| 1 | 1 | sp\|P50454\|SERPH_HUMAN | SERPINH1 | 46.41 | 2.8283 |
| 1 | 1 | sp\|P56192\|SYMC_HUMAN | MARS | 101.05 | 2.8127 |
| 1 | 1 | sp\|Q5JU69\|TOR2A_HUMAN | TOR2A | 35.69 | 2.8096 |
| 1 | 1 | sp\|Q9Y232\|CDYL1_HUMAN | CDYL | 66.44 | 2.7991 |
| 1 | 1 | sp\|P50897\|PPT1_HUMAN | PPT1 | 34.17 | 2.7912 |
| 1 | 1 | sp\|Q9NZ08\|ERAP1_HUMAN | ERAP1 | 107.17 | 2.7866 |
| 1 | 1 | sp\|Q9NQ50\|RM40_HUMAN | MRPL40 | 24.48 | 2.7723 |
| 1 | 1 | sp\|Q96GQ7\|DDX27_HUMAN | DDX27 | 89.78 | 2.766 |
| 1 | 1 | sp\|O14735\|CDIPT_HUMAN | CDIPT | 23.52 | 2.7651 |
| 1 | 1 | sp\|O95409\|ZIC2_HUMAN | ZIC2 | 54.97 | 2.7592 |
| 1 | 1 | sp\|Q3SXM5\|HSDL1_HUMAN | HSDL1 | 36.98 | 2.7545 |
| 1 | 1 | sp\|Q86Y39\|NDUAB_HUMAN | NDUFA11 | 14.84 | 2.7501 |
| 1 | 1 | sp\|Q9UBF2\|COPG2_HUMAN | COPG2 | 97.56 | 2.7403 |
| 1 | 1 | sp\|Q8TCJ2\|STT3B_HUMAN | STT3B | 93.61 | 2.736 |
| 1 | 1 | sp\|O14802\|RPC1_HUMAN | POLR3A | 155.54 | 2.7169 |
| 1 | 1 | sp\|Q15269\|PWP2_HUMAN | PWP2 | 102.39 | 2.711 |
| 1 | 1 | KV2A7_MOUSE | | 12.27 | 2.7025 |
| 1 | 1 | sp\|Q12830\|BPTF_HUMAN | BPTF | 338.05 | 2.6977 |
| 1 | 1 | sp\|Q8N8L6\|ARL10_HUMAN | ARL10 | 27.44 | 2.6741 |
| 1 | 1 | sp\|Q9Y697\|NFS1_HUMAN | NFS1 | 50.16 | 2.6592 |
| 1 | 1 | sp\|Q92797\|SYMPK_HUMAN | SYMPK | 141.06 | 2.6557 |
| 1 | 1 | sp\|Q9UI10\|EI2BD_HUMAN | EIF2B4 | 57.52 | 2.6485 |
| 1 | 1 | sp\|Q9BVQ7\|SPA5L_HUMAN | SPATA5L1 | 80.66 | 2.6331 |
| 1 | 1 | sp\|Q15388\|TOM20_HUMAN | TOMM20 | 16.29 | 2.6315 |
| 1 | 1 | sp\|P82933\|RT09_HUMAN | MRPS9 | 45.81 | 2.6192 |
| 1 | 1 | sp\|Q9BXW9\|FACD2_HUMAN | FANCD2 | 164.02 | 2.6144 |
| 1 | 1 | sp\|Q6PML9\|ZNT9_HUMAN | SLC30A9 | 63.47 | 2.6142 |
| 1 | 1 | sp\|Q8TBF5\|PIGX_HUMAN | PIGX | 28.77 | 2.6142 |
| 1 | 1 | sp\|Q9Y5J1\|UTP18_HUMAN | UTP18 | 61.96 | 2.6063 |
| 1 | 1 | sp\|Q86TJ2\|TAD2B_HUMAN | TADA2B | 48.44 | 2.5991 |
| 1 | 1 | sp\|P68366\|TBA4A_HUMAN | TUBA4A | 49.89 | 2.5969 |
| 1 | 1 | sp\|Q6DRA6\|H2B2D_HUMAN | HIST2H2BD | 18.01 | 2.5889 |
| 1 | 1 | sp\|Q9BWM7\|SFXN3_HUMAN | SFXN3 | 35.48 | 2.5811 |
| 1 | 1 | sp\|Q96H55\|MYO19_HUMAN | MYO19 | 109.07 | 2.5717 |
| 1 | 1 | sp\|Q9Y4F1\|FARP1_HUMAN | FARP1 | 118.56 | 2.5565 |
| 1 | 1 | sp\|Q8N684\|CPSF7_HUMAN | CPSF7 | 52.02 | 2.5537 |
| 1 | 1 | sp\|Q8NFQ8\|TOIP2_HUMAN | TOR1AIP2 | 51.23 | 2.5378 |
| 1 | 1 | sp\|O43823\|AKAP8_HUMAN | AKAP8 | 76.06 | 2.5116 |
| 1 | 1 | sp\|O15260\|SURF4_HUMAN | SURF4 | 30.37 | 2.5109 |
| 1 | 1 | sp\|P52948\|NUP98_HUMAN | NUP98 | 197.46 | 2.5109 |
| 1 | 1 | sp\|Q8WVM8\|SCFD1_HUMAN | SCFD1 | 72.33 | 2.4935 |
| 1 | 1 | sp\|P14678\|RSMB_HUMAN | SNRPB | 24.59 | 2.483 |
| 1 | 1 | sp\|Q12962\|TAF10_HUMAN | TAF10 | 21.7 | 2.4811 |
| 1 | 1 | sp\|Q8NB90\|SPAT5_HUMAN | SPATA5 | 97.84 | 2.4769 |
| 1 | 1 | tr\|Q96DP0\|Q96DP0_HUMAN | | 49.22 | 2.4718 |
| 1 | 1 | sp\|Q8NBU5\|ATAD1_HUMAN | ATAD1 | 40.72 | 2.4518 |
| 1 | 1 | sp\|P10515\|ODP2_HUMAN | DLAT | 68.95 | 2.4454 |
| 1 | 1 | sp\|Q643R3\|LPCT4_HUMAN | LPCAT4 | 57.18 | 2.4415 |
| 1 | 1 | sp\|P23258\|TBG1_HUMAN | TUBG1 | 51.14 | 2.4387 |
| 1 | 1 | sp\|Q9Y6K0\|CEPT1_HUMAN | CEPT1 | 46.52 | 2.4294 |
| 1 | 1 | sp\|Q9Y230\|RUVB2_HUMAN | RUVBL2 | 51.12 | 2.4188 |
| 1 | 1 | sp\|O60725\|ICMT_HUMAN | ICMT | 31.92 | 2.4021 |
| 1 | 1 | sp\|Q9BYW2\|SETD2_HUMAN | SETD2 | 287.42 | 2.3755 |
| 1 | 1 | sp\|Q8N4U5\|T11L2_HUMAN | TCP11L2 | 58.05 | 2.3706 |
| 1 | 1 | sp\|Q9H2V7\|SPNS1_HUMAN | SPNS1 | 56.59 | 2.3667 |
| 1 | 1 | sp\|Q9BSK2\|S2533_HUMAN | SLC25A33 | 35.35 | 2.3493 |
| 1 | 1 | sp\|P51530\|DNA2_HUMAN | DNA2 | 120.34 | 2.3388 |
| 1 | 1 | sp\|Q9Y265\|RUVB1_HUMAN | RUVBL1 | 50.2 | 2.3132 |
| 1 | 1 | sp\|Q15043\|S39AE_HUMAN | SLC39A14 | 54.18 | 2.3094 |
| 1 | 1 | sp\|Q6P4A7\|SFXN4_HUMAN | SFXN4 | 37.97 | 2.3065 |
| 1 | 1 | sp\|Q92878\|RAD50_HUMAN | RAD50 | 153.8 | 2.2886 |
| 1 | 1 | sp\|P31327\|CPSM_HUMAN | CPS1 | 164.83 | 2.2879 |
| 1 | 1 | sp\|P61353\|RL27_HUMAN | RPL27 | 15.79 | 2.2777 |
| 1 | 1 | sp\|Q8N6I1\|EID2_HUMAN | EID2 | 25.17 | 2.2718 |
| 1 | 1 | sp\|Q9NZJ7\|MTCH1_HUMAN | MTCH1 | 41.52 | 2.2539 |
| 1 | 1 | sp\|P33121\|ACSL1_HUMAN | ACSL1 | 77.89 | 2.2518 |
| 1 | 1 | sp\|Q8NHH9\|ATLA2_HUMAN | ATL2 | 66.19 | 2.2317 |
| 1 | 1 | sp\|Q7L2E3\|DHX30_HUMAN | DHX30 | 133.85 | 2.2277 |
| 1 | 1 | sp\|Q6VAB6\|KSR2_HUMAN | KSR2 | 107.56 | 2.2255 |
| 1 | 1 | sp\|Q9BVK2\|ALG8_HUMAN | ALG8 | 60.05 | 2.2052 |
| 1 | 1 | sp\|P35914\|HMGCL_HUMAN | HMGCL | 34.34 | 2.1941 |
| 1 | 1 | sp\|Q15526\|SURF1_HUMAN | SURF1 | 33.31 | 2.1791 |
| 1 | 1 | sp\|O14681\|EI24_HUMAN | EI24 | 38.94 | 2.1725 |
| 1 | 1 | sp\|Q9ULD4\|BRPF3_HUMAN | BRPF3 | 135.66 | 2.1675 |
| 1 | 1 | sp\|Q02539\|H11_HUMAN | HIST1H1A | 21.83 | 2.15 |

TABLE 5C
HA-SS18WT_CHR_peptides

| Unique | Total | reference | Gene Symbol | MWT(kDa) | AVG |
|---|---|---|---|---|---|
| 43 | 156 | sp\|P51532\|SMCA4_HUMAN | SMARCA4 | 184.53 | 3.0834 |
| 46 | 128 | sp\|P51531\|SMCA2_HUMAN | SMARCA2 | 181.17 | 2.9702 |
| 8 | 108 | sp\|P33778\|H2B1B_HUMAN | HIST1H2BB | 13.94 | 2.3822 |
| 45 | 69 | sp\|O14497\|ARI1A_HUMAN | ARID1A | 241.89 | 3.2558 |
| 24 | 52 | sp\|Q8TAQ2\|SMRC2_HUMAN | SMARCC2 | 132.8 | 3.3159 |
| 34 | 50 | sp\|Q8NFD5\|ARI1B_HUMAN | ARID1B | 235.97 | 3.1174 |
| 24 | 43 | sp\|Q92922\|SMRC1_HUMAN | SMARCC1 | 122.79 | 3.4232 |
| 5 | 40 | sp\|Q96KK5\|H2A1H_HUMAN | HIST1H2AH | 13.9 | 2.7079 |
| 31 | 33 | sp\|Q14839\|CHD4_HUMAN | CHD4 | 217.87 | 3.3364 |
| 24 | 33 | sp\|Q08211\|DHX9_HUMAN | DHX9 | 140.87 | 3.1075 |
| 29 | 31 | sp\|Q14980\|NUMA1_HUMAN | NUMA1 | 238.12 | 3.7685 |
| 28 | 31 | sp\|Q6P2Q9\|PRP8_HUMAN | PRPF8 | 273.43 | 3.1678 |
| 21 | 31 | sp\|Q9NYF8\|BCLF1_HUMAN | BCLAF1 | 106.06 | 3.1061 |
| 29 | 30 | sp\|O75643\|U520_HUMAN | SNRNP200 | 244.35 | 3.4233 |
| 20 | 30 | sp\|Q6STE5\|SMRD3_HUMAN | SMARCD3 | 54.98 | 3.0867 |
| 24 | 29 | sp\|P11388\|TOP2A_HUMAN | TOP2A | 174.28 | 3.2664 |
| 15 | 29 | sp\|Q96GM5\|SMRD1_HUMAN | SMARCD1 | 58.2 | 3.3365 |
| 17 | 27 | sp\|Q00839\|HNRPU_HUMAN | HNRNPU | 90.53 | 3.1686 |
| 15 | 27 | sp\|P52272\|HNRPM_HUMAN | HNRNPM | 77.46 | 3.1827 |
| 11 | 27 | sp\|P22087\|FBRL_HUMAN | FBL | 33.76 | 3.0616 |
| 25 | 26 | sp\|O75691\|UTP20_HUMAN | UTP20 | 318.18 | 2.9961 |
| 25 | 25 | sp\|Q9H583\|HEAT1_HUMAN | HEATR1 | 242.22 | 3.3547 |
| 24 | 25 | sp\|Q8WYP5\|ELYS_HUMAN | AHCTF1 | 252.34 | 3.4786 |
| 9 | 25 | sp\|P62805\|H4_HUMAN | HIST1H4A | 11.36 | 2.9256 |
| 14 | 24 | sp\|Q9Y2X3\|NOP58_HUMAN | NOP58 | 59.54 | 3.8372 |
| 15 | 23 | sp\|Q9Y2W1\|TR150_HUMAN | THRAP3 | 108.6 | 2.9034 |
| 22 | 22 | sp\|Q14690\|RRP5_HUMAN | PDCD11 | 208.57 | 3.3884 |
| 18 | 22 | sp\|O00567\|NOP56_HUMAN | NOP56 | 66.01 | 3.6951 |
| 20 | 21 | sp\|Q9Y5B9\|SP16H_HUMAN | SUPT16H | 119.84 | 3.3847 |
| 13 | 21 | sp\|O76021\|RL1D1_HUMAN | RSL1D1 | 54.94 | 2.4994 |
| 12 | 21 | sp\|O96019\|ACL6A_HUMAN | ACTL6A | 47.43 | 3.0129 |
| 18 | 20 | sp\|Q02880\|TOP2B_HUMAN | TOP2B | 183.15 | 3.2243 |
| 19 | 19 | sp\|Q9UIG0\|BAZ1B_HUMAN | BAZ1B | 170.8 | 3.4521 |
| 16 | 19 | sp\|P08670\|VIME_HUMAN | VIM | 53.62 | 3.4552 |
| 10 | 19 | sp\|Q12824\|SNF5_HUMAN | SMARCB1 | 44.11 | 3.4839 |
| 18 | 18 | sp\|Q9NTI5\|PDS5B_HUMAN | PDS5B | 164.56 | 2.7474 |
| 16 | 17 | sp\|Q9H0A0\|NAT10_HUMAN | NAT10 | 115.66 | 3.0877 |
| 14 | 17 | sp\|Q969G3\|SMCE1_HUMAN | SMARCE1 | 46.62 | 3.741 |
| 6 | 17 | sp\|P62736\|ACTA_HUMAN | ACTA2 | 41.98 | 2.916 |
| 11 | 16 | sp\|Q9H307\|PININ_HUMAN | PNN | 81.56 | 3.2407 |
| 10 | 16 | sp\|P43243\|MATR3_HUMAN | MATR3 | 94.56 | 3.0893 |
| 6 | 16 | sp\|P63261\|ACTG_HUMAN | ACTG1 | 41.77 | 3.2621 |
| 15 | 15 | sp\|Q03164\|KMT2A_HUMAN | KMT2A | 431.5 | 3.5526 |
| 15 | 15 | sp\|O75533\|SF3B1_HUMAN | SF3B1 | 145.74 | 3.4533 |
| 14 | 15 | sp\|O60264\|SMCA5_HUMAN | SMARCA5 | 121.83 | 2.9348 |
| 13 | 15 | sp\|Q08945\|SSRP1_HUMAN | SSRP1 | 81.02 | 3.2569 |
| 13 | 15 | sp\|Q9Y3T9\|NOC2L_HUMAN | NOC2L | 84.87 | 3.1816 |
| 6 | 15 | sp\|P07910\|HNRPC_HUMAN | HNRNPC | 33.65 | 2.9851 |
| 14 | 14 | sp\|Q6PL18\|ATAD2_HUMAN | ATAD2 | 158.46 | 3.699 |
| 13 | 14 | sp\|Q14683\|SMC1A_HUMAN | SMC1A | 143.14 | 3.4429 |
| 13 | 14 | sp\|Q96GQ7\|DDX27_HUMAN | DDX27 | 89.78 | 3.3349 |
| 13 | 14 | sp\|Q9UQE7\|SMC3_HUMAN | SMC3 | 141.45 | 3.3166 |
| 11 | 14 | sp\|Q12905\|ILF2_HUMAN | ILF2 | 43.04 | 3.537 |
| 11 | 14 | sp\|Q13601\|KRR1_HUMAN | KRR1 | 43.64 | 3.053 |
| 11 | 14 | sp\|Q9BQG0\|MBB1A_HUMAN | MYBBP1A | 148.76 | 2.9411 |
| 5 | 14 | IGH1M_MOUSE | lghg1 | 43.36 | 2.7484 |
| 13 | 13 | sp\|P20700\|LMNB1_HUMAN | LMNB1 | 66.37 | 3.6005 |
| 13 | 13 | sp\|P78527\|PRKDC_HUMAN | PRKDC | 468.79 | 3.1715 |
| 12 | 13 | sp\|Q9UKV3\|ACINU_HUMAN | ACIN1 | 151.77 | 3.2842 |
| 11 | 13 | sp\|Q92925\|SMRD2_HUMAN | SMARCD2 | 58.88 | 3.4743 |
| 11 | 13 | sp\|Q9ULI0\|ATD2B_HUMAN | ATAD2B | 164.81 | 2.9966 |
| 10 | 13 | sp\|Q92785\|REQU_HUMAN | DPF2 | 44.13 | 3.5859 |
| 8 | 13 | sp\|Q14978\|NOLC1_HUMAN | NOLC1 | 73.56 | 2.752 |
| 12 | 12 | sp\|Q8IY81\|SPB1_HUMAN | FTSJ3 | 96.5 | 3.5724 |
| 12 | 12 | sp\|P33993\|MCM7_HUMAN | MCM7 | 81.26 | 3.0588 |
| 11 | 12 | sp\|Q8WWQ0\|PHIP_HUMAN | PHIP | 206.56 | 3.385 |
| 10 | 12 | sp\|P46087\|NOP2_HUMAN | NOP2 | 89.25 | 3.5911 |
| 9 | 12 | sp\|Q9H2P0\|ADNP_HUMAN | ADNP | 123.49 | 3.844 |
| 10 | 11 | sp\|Q9NZM4\|BICRA_HUMAN | BICRA | 158.39 | 3.6731 |
| 10 | 11 | sp\|Q15029\|U5S1_HUMAN | EFTUD2 | 109.37 | 3.5515 |
| 10 | 11 | sp\|P18583\|SON_HUMAN | SON | 263.66 | 3.4212 |
| 10 | 10 | sp\|Q7Z3K3\|POGZ_HUMAN | POGZ | 155.24 | 3.1926 |
| 10 | 10 | sp\|Q6KC79\|NIPBL_HUMAN | NIPBL | 315.85 | 3.1909 |
| 10 | 10 | sp\|P46013\|KI67_HUMAN | MKI67 | 358.47 | 2.9203 |
| 9 | 10 | sp\|Q12906\|ILF3_HUMAN | ILF3 | 95.28 | 3.5285 |
| 9 | 10 | sp\|Q86U86\|PB1_HUMAN | PBRM1 | 192.83 | 3.1412 |
| 9 | 10 | sp\|Q96T58\|MINT_HUMAN | SPEN | 402 | 3.1272 |
| 9 | 10 | sp\|O60216\|RAD21_HUMAN | RAD21 | 71.64 | 3.0948 |
| 8 | 10 | sp\|Q9Y2R4\|DDX52_HUMAN | DDX52 | 67.46 | 3.9244 |
| 7 | 10 | sp\|Q07955\|SRSF1_HUMAN | SRSF1 | 27.73 | 2.7409 |
| 9 | 9 | sp\|O14646\|CHD1_HUMAN | CHD1 | 196.57 | 4.0207 |
| 9 | 9 | sp\|Q99459\|CDC5L_HUMAN | CDC5L | 92.19 | 3.7929 |
| 9 | 9 | sp\|O43143\|DHX15_HUMAN | DHX15 | 90.88 | 3.4603 |
| 9 | 9 | sp\|O75367\|H2AY_HUMAN | H2AFY | 39.59 | 3.3062 |
| 9 | 9 | sp\|Q15393\|SF3B3_HUMAN | SF3B3 | 135.49 | 3.3021 |
| 9 | 9 | sp\|P24928\|RPB1_HUMAN | POLR2A | 217.04 | 3.2373 |
| 9 | 9 | sp\|Q9NVP1\|DDX18_HUMAN | DDX18 | 75.36 | 3.2151 |
| 9 | 9 | sp\|Q8IXT5\|RB12B_HUMAN | RBM12B | 118.03 | 3.1281 |
| 9 | 9 | sp\|P17480\|UBF1_HUMAN | UBTF | 89.35 | 3.0431 |
| 9 | 9 | sp\|Q9NR30\|DDX21_HUMAN | DDX21 | 87.29 | 2.897 |
| 9 | 9 | sp\|Q9UJV9\|DDX41_HUMAN | DDX41 | 69.79 | 2.7643 |
| 7 | 9 | sp\|Q96T23\|RSF1_HUMAN | RSF1 | 163.72 | 3.2299 |
| 7 | 9 | sp\|P55795\|HNRH2_HUMAN | HNRNPH2 | 49.23 | 2.8092 |
| 6 | 9 | sp\|Q86VM9\|ZCH18_HUMAN | ZC3H18 | 106.32 | 2.8814 |
| 5 | 9 | sp\|Q8WUZ0\|BCL7C_HUMAN | BCL7C | 23.45 | 3.9306 |
| 4 | 9 | sp\|Q4VC05\|BCL7A_HUMAN | BCL7A | 22.8 | 3.6922 |
| 8 | 8 | sp\|Q8WTT2\|NOC3L_HUMAN | NOC3L | 92.49 | 3.9612 |
| 8 | 8 | sp\|P55265\|DSRAD_HUMAN | ADAR | 135.98 | 3.7284 |
| 8 | 8 | sp\|Q8TDD1\|DDX54_HUMAN | DDX54 | 98.53 | 3.4328 |
| 8 | 8 | sp\|Q9UQ35\|SRRM2_HUMAN | SRRM2 | 299.44 | 3.0215 |
| 7 | 8 | sp\|Q9NY61\|AATF_HUMAN | AATF | 63.09 | 3.6956 |
| 7 | 8 | sp\|Q1KMD3\|HNRL2_HUMAN | HNRNPUL2 | 85.05 | 3.1539 |
| 6 | 8 | sp\|Q49A26\|GLYR1_HUMAN | GLYR1 | 60.52 | 3.8419 |
| 6 | 8 | sp\|Q9P0M6\|H2AW_HUMAN | H2AFY2 | 40.03 | 3.6549 |
| 6 | 8 | sp\|B2RXH8\|HNRC2_HUMAN | HNRNPCL2 | 32.05 | 2.8517 |
| 7 | 7 | sp\|Q13435\|SF3B2_HUMAN | SF3B2 | 100.16 | 3.6675 |
| 7 | 7 | sp\|Q14676\|MDC1_HUMAN | MDC1 | 226.53 | 3.6627 |
| 7 | 7 | sp\|Q9H6F5\|CCD86_HUMAN | CCDC86 | 40.21 | 3.6182 |
| 7 | 7 | sp\|Q12788\|TBL3_HUMAN | TBL3 | 88.98 | 3.609 |
| 7 | 7 | sp\|Q9H8M2\|BRD9_HUMAN | BRD9 | 66.96 | 3.5025 |
| 7 | 7 | sp\|Q8TDL5\|BPIB1_HUMAN | BPIFB1 | 52.41 | 3.4522 |
| 7 | 7 | sp\|P50402\|EMD_HUMAN | EMD | 28.98 | 3.4145 |
| 7 | 7 | sp\|P19338\|NUCL_HUMAN | NCL | 76.57 | 3.3742 |
| 7 | 7 | sp\|Q12873\|CHD3_HUMAN | CHD3 | 226.45 | 3.1365 |
| 7 | 7 | sp\|Q9BZE4\|NOG1_HUMAN | GTPBP4 | 73.92 | 3.0581 |
| 7 | 7 | sp\|Q8IWA0\|WDR75_HUMAN | WDR75 | 94.44 | 3.0082 |
| 7 | 7 | sp\|P28370\|SMCA1_HUMAN | SMARCA1 | 122.53 | 2.9683 |
| 7 | 7 | sp\|Q92841\|DDX17_HUMAN | DDX17 | 80.22 | 2.9366 |
| 7 | 7 | sp\|Q8N1F7\|NUP93_HUMAN | NUP93 | 93.43 | 2.9068 |
| 7 | 7 | sp\|Q6ZRS2\|SRCAP_HUMAN | SRCAP | 343.34 | 2.8432 |
| 7 | 7 | sp\|Q03188\|CENPC_HUMAN | CENPC | 106.77 | 2.6862 |
| 6 | 7 | sp\|O00566\|MPP10_HUMAN | MPHOSPH10 | 78.82 | 3.9439 |
| 6 | 7 | sp\|Q9H9B4\|SFXN1_HUMAN | SFXN1 | 35.6 | 3.5359 |
| 6 | 7 | sp\|O60306\|AQR_HUMAN | AQR | 171.19 | 3.4683 |
| 6 | 7 | sp\|P62995\|TRA2B_HUMAN | TRA2B | 33.65 | 3.0926 |
| 6 | 7 | sp\|Q8N8A6\|DDX51_HUMAN | DDX51 | 72.41 | 3.0592 |
| 5 | 7 | sp\|Q5QJE6\|TDIF2_HUMAN | DNTTIP2 | 84.42 | 3.1584 |
| 4 | 7 | sp\|P16403\|H12_HUMAN | HIST1H1C | 21.35 | 3.2204 |
| 6 | 6 | sp\|Q9HCS7\|SYF1_HUMAN | XAB2 | 99.95 | 3.9611 |
| 6 | 6 | sp\|Q9Y5B6\|PAXB1_HUMAN | PAXBP1 | 104.74 | 3.8492 |
| 6 | 6 | sp\|O60281\|ZN292_HUMAN | ZNF292 | 304.62 | 3.8342 |
| 6 | 6 | sp\|Q14692\|BMS1_HUMAN | BMS1 | 145.72 | 3.8277 |
| 6 | 6 | sp\|Q9BVP2\|GNL3_HUMAN | GNL3 | 61.95 | 3.8227 |
| 6 | 6 | sp\|Q14202\|ZMYM3_HUMAN | ZMYM3 | 152.28 | 3.5194 |
| 6 | 6 | sp\|Q8N3U4\|STAG2_HUMAN | STAG2 | 141.24 | 3.5054 |
| 6 | 6 | sp\|Q9NVH2\|INT7_HUMAN | INTS7 | 106.77 | 3.419 |
| 6 | 6 | sp\|P49792\|RBP2_HUMAN | RANBP2 | 357.97 | 3.3617 |
| 6 | 6 | sp\|Q9UK61\|TASOR_HUMAN | FAM208A | 188.91 | 3.0003 |
| 6 | 6 | sp\|Q9UKM9\|RALY_HUMAN | RALY | 32.44 | 2.8877 |
| 6 | 6 | sp\|Q96PK6\|RBM14_HUMAN | RBM14 | 69.45 | 2.8047 |
| 5 | 6 | sp\|O00159\|MYO1C_HUMAN | MYO1C | 121.61 | 4.3268 |
| 5 | 6 | sp\|Q6NSZ9\|ZSC25_HUMAN | ZSCAN25 | 61.44 | 3.5224 |
| 5 | 6 | sp\|Q7Z7K6\|CENPV_HUMAN | CENPV | 29.93 | 3.357 |
| 5 | 6 | sp\|P68371\|TBB4B_HUMAN | TUBB4B | 49.8 | 3.1922 |
| 5 | 6 | sp\|Q9BVJ6\|UT14A_HUMAN | UTP14A | 87.92 | 3.1504 |
| 5 | 6 | sp\|Q96KR1\|ZFR_HUMAN | ZFR | 116.94 | 2.9326 |
| 5 | 6 | sp\|P62277\|RS13_HUMAN | RPS13 | 17.21 | 2.654 |
| 4 | 6 | sp\|P31943\|HNRH1_HUMAN | HNRNPH1 | 49.2 | 3.278 |
| 4 | 6 | sp\|Q16629\|SRSF7_HUMAN | SRSF7 | 27.35 | 2.6251 |
| 2 | 6 | sp\|Q5BKZ1\|ZN326_HUMAN | ZNF326 | 65.61 | 2.8796 |
| 2 | 6 | sp\|Q93079\|H2B1H_HUMAN | HIST1H2BH | 13.88 | 2.0968 |
| 5 | 5 | sp\|Q99549\|MPP8_HUMAN | MPHOSPH8 | 97.12 | 4.0198 |
| 5 | 5 | sp\|P33991\|MCM4_HUMAN | MCM4 | 96.5 | 3.8766 |
| 5 | 5 | sp\|Q14137\|BOP1_HUMAN | BOP1 | 83.58 | 3.8261 |
| 5 | 5 | sp\|Q15269\|PWP2_HUMAN | PWP2 | 102.39 | 3.7907 |
| 5 | 5 | sp\|Q03701\|CEBPZ_HUMAN | CEBPZ | 120.9 | 3.7691 |
| 5 | 5 | sp\|P57740\|NU107_HUMAN | NUP107 | 106.31 | 3.6565 |
| 5 | 5 | sp\|Q86YP4\|P66A_HUMAN | GATAD2A | 68.02 | 3.5678 |
| 5 | 5 | sp\|Q9BSC4\|NOL10_HUMAN | NOL10 | 80.25 | 3.5655 |
| 5 | 5 | sp\|Q8TED0\|UTP15_HUMAN | UTP15 | 58.38 | 3.526 |
| 5 | 5 | sp\|P25440\|BRD2_HUMAN | BRD2 | 88.01 | 3.4882 |
| 5 | 5 | sp\|Q9NXF1\|TEX10_HUMAN | TEX10 | 105.61 | 3.4606 |
| 5 | 5 | sp\|Q15459\|SF3A1_HUMAN | SF3A1 | 88.83 | 3.4356 |
| 5 | 5 | sp\|P07199\|CENPB_HUMAN | CENPB | 65.13 | 3.4356 |
| 5 | 5 | sp\|Q9BYG3\|MK67I_HUMAN | NIFK | 34.2 | 3.296 |
| 5 | 5 | sp\|Q8N7H5\|PAF1_HUMAN | PAF1 | 59.94 | 3.2547 |
| 5 | 5 | sp\|Q13620\|CUL4B_HUMAN | CUL4B | 103.92 | 3.2437 |
| 5 | 5 | sp\|P52701\|MSH6_HUMAN | MSH6 | 152.69 | 3.2215 |
| 5 | 5 | sp\|P62424\|RL7A_HUMAN | RPL7A | 29.98 | 3.2058 |
| 5 | 5 | sp\|Q15397\|PUM3_HUMAN | PUM3 | 73.54 | 3.2016 |
| 5 | 5 | sp\|Q9NQS7\|INCE_HUMAN | INCENP | 105.36 | 3.0804 |
| 5 | 5 | sp\|Q9H8H0\|NOL11_HUMAN | NOL11 | 81.07 | 2.9959 |
| 5 | 5 | sp\|O43390\|HNRPR_HUMAN | HNRNPR | 70.9 | 2.9694 |
| 5 | 5 | sp\|Q96H22\|CENPN_HUMAN | CENPN | 39.53 | 2.9578 |
| 5 | 5 | sp\|P45880\|VDAC2_HUMAN | VDAC2 | 31.55 | 2.9465 |
| 5 | 5 | sp\|Q6AI39\|BICRL_HUMAN | BICRAL | 115.01 | 2.8687 |
| 5 | 5 | sp\|P46100\|ATRX_HUMAN | ATRX | 282.41 | 2.8152 |
| 5 | 5 | sp\|Q9UIF9\|BAZ2A_HUMAN | BAZ2A | 211.07 | 2.7088 |
| 4 | 5 | sp\|Q9Y5J1\|UTP18_HUMAN | UTP18 | 61.96 | 3.2081 |
| 4 | 5 | sp\|Q92794\|KAT6A_HUMAN | KAT6A | 224.89 | 2.736 |
| 2 | 5 | sp\|Q15532\|SSXT_HUMAN | SS18 | 45.9 | 3.3233 |
| 1 | 5 | tr\|Q6PJV4\|Q6PJV4_HUMAN | THRAP3 | 41.83 | 3.8114 |
| 4 | 4 | sp\|P61978\|HNRPK_HUMAN | HNRNPK | 50.94 | 4.6312 |
| 4 | 4 | sp\|O96028\|NSD2_HUMAN | NSD2 | 152.16 | 4.0798 |
| 4 | 4 | sp\|Q9NYH9\|UTP6_HUMAN | UTP6 | 70.15 | 3.9237 |
| 4 | 4 | sp\|Q12931\|TRAP1_HUMAN | TRAP1 | 80.06 | 3.661 |
| 4 | 4 | sp\|Q16531\|DDB1_HUMAN | DDB1 | 126.89 | 3.4707 |
| 4 | 4 | sp\|Q15287\|RNPS1_HUMAN | RNPS1 | 34.19 | 3.4291 |
| 4 | 4 | sp\|P51398\|RT29_HUMAN | DAP3 | 45.54 | 3.3906 |
| 4 | 4 | sp\|P05412\|JUN_HUMAN | JUN | 35.65 | 3.3333 |
| 4 | 4 | sp\|P15880\|RS2_HUMAN | RPS2 | 31.3 | 3.2743 |
| 4 | 4 | sp\|P38159\|RBMX_HUMAN | RBMX | 42.31 | 3.2614 |
| 4 | 4 | sp\|Q8WXH0\|SYNE2_HUMAN | SYNE2 | 795.94 | 3.2386 |
| 4 | 4 | sp\|Q9Y4W2\|LAS1L_HUMAN | LAS1L | 83.01 | 3.218 |
| 4 | 4 | sp\|Q8WXI9\|P66B_HUMAN | GATAD2B | 65.22 | 3.2163 |
| 4 | 4 | sp\|P21796\|VDAC1_HUMAN | VDAC1 | 30.75 | 3.1761 |
| 4 | 4 | sp\|O94776\|MTA2_HUMAN | MTA2 | 74.98 | 3.1723 |
| 4 | 4 | sp\|Q16891\|MIC60_HUMAN | IMMT | 83.63 | 3.1454 |
| 4 | 4 | sp\|Q9Y2P8\|RCL1_HUMAN | RCL1 | 40.82 | 3.1052 |
| 4 | 4 | sp\|O75400\|PR40A_HUMAN | PRPF40A | 108.74 | 3.1025 |
| 4 | 4 | sp\|Q9UJS0\|CMC2_HUMAN | SLC25A13 | 74.13 | 3.0994 |
| 4 | 4 | sp\|P06733\|ENOA_HUMAN | ENO1 | 47.14 | 3.0929 |
| 4 | 4 | sp\|O60287\|NPA1P_HUMAN | URB1 | 254.23 | 3.0688 |
| 4 | 4 | sp\|P11498\|PYC_HUMAN | PC | 129.55 | 3.0253 |
| 4 | 4 | sp\|P11021\|GRP78_HUMAN | HSPA5 | 72.29 | 2.9973 |
| 4 | 4 | sp\|Q92621\|NU205_HUMAN | NUP205 | 227.78 | 2.9481 |
| 4 | 4 | sp\|P06576\|ATPB_HUMAN | ATP5B | 56.52 | 2.8745 |
| 4 | 4 | sp\|Q68CP9\|ARID2_HUMAN | ARID2 | 197.27 | 2.8522 |
| 4 | 4 | sp\|P18124\|RL7_HUMAN | RPL7 | 29.21 | 2.7909 |
| 4 | 4 | sp\|Q9H582\|ZN644_HUMAN | ZNF644 | 149.47 | 2.7228 |
| 4 | 4 | sp\|P17844\|DDX5_HUMAN | DDX5 | 69.1 | 2.7058 |
| 4 | 4 | sp\|Q96ME7\|ZN512_HUMAN | ZNF512 | 64.64 | 2.6681 |
| 4 | 4 | sp\|Q5VWN6\|F208B_HUMAN | FAM208B | 268.68 | 2.5746 |
| 4 | 4 | sp\|P36578\|RL4_HUMAN | RPL4 | 47.67 | 2.534 |
| 4 | 4 | sp\|P49756\|RBM25_HUMAN | RBM25 | 100.12 | 2.5337 |
| 4 | 4 | sp\|P49750\|YLPM1_HUMAN | YLPM1 | 219.85 | 2.2901 |
| 3 | 4 | tr\|B2R5W2\|B2R5W2_HUMAN | HNRNPC | 31.93 | 4.877 |
| 3 | 4 | sp\|P62701\|RS4X_HUMAN | RPS4X | 29.58 | 3.4819 |
| 3 | 4 | sp\|Q96KQ7\|EHMT2_HUMAN | EHMT2 | 132.29 | 3.1695 |
| 3 | 4 | sp\|P62987\|RL40_HUMAN | UBA52 | 14.72 | 3.1637 |
| 3 | 4 | sp\|Q9NU22\|MDN1_HUMAN | MDN1 | 632.42 | 3.0818 |
| 3 | 4 | sp\|Q9H501\|ESF1_HUMAN | ESF1 | 98.73 | 2.9547 |
| 3 | 4 | IGKC_MOUSE | | 11.77 | 2.9149 |
| 3 | 4 | sp\|Q12769\|NU160_HUMAN | NUP160 | 162.02 | 2.8295 |
| 3 | 4 | sp\|Q5JTH9\|RRP12_HUMAN | RRP12 | 143.61 | 2.7939 |
| 3 | 4 | sp\|Q9Y277\|VDAC3_HUMAN | VDAC3 | 30.64 | 2.7441 |
| 3 | 4 | sp\|Q13247\|SRSF6_HUMAN | SRSF6 | 39.56 | 2.6268 |
| 2 | 4 | sp\|Q96PV6\|LENG8_HUMAN | LENG8 | 86.07 | 2.0279 |
| 3 | 3 | sp\|Q15061\|WDR43_HUMAN | WDR43 | 74.84 | 4.5687 |
| 3 | 3 | sp\|Q13185\|CBX3_HUMAN | CBX3 | 20.8 | 4.3351 |
| 3 | 3 | sp\|Q969X6\|UTP4_HUMAN | UTP4 | 76.84 | 4.238 |
| 3 | 3 | sp\|Q7L2E3\|DHX30_HUMAN | DHX30 | 133.85 | 4.2339 |
| 3 | 3 | sp\|Q9Y3C1\|NOP16_HUMAN | NOP16 | 21.18 | 4.2066 |
| 3 | 3 | sp\|A8CG34\|P121C_HUMAN | POM121C | 124.98 | 4.0062 |
| 3 | 3 | sp\|P25705\|ATPA_HUMAN | ATP5A1 | 59.71 | 3.966 |
| 3 | 3 | sp\|Q9BQE3\|TBA1C_HUMAN | TUBA1C | 49.86 | 3.8249 |
| 3 | 3 | sp\|Q5SSJ5\|HP1B3_HUMAN | HP1BP3 | 61.17 | 3.8158 |
| 3 | 3 | sp\|P62753\|RS6_HUMAN | RPS6 | 28.66 | 3.8152 |
| 3 | 3 | sp\|Q96QD9\|UIF_HUMAN | FYTTD1 | 35.8 | 3.7604 |
| 3 | 3 | sp\|P52597\|HNRPF_HUMAN | HNRNPF | 45.64 | 3.7157 |
| 3 | 3 | sp\|Q9BUJ2\|HNRL1_HUMAN | HNRNPUL1 | 95.68 | 3.6878 |
| 3 | 3 | sp\|P31942\|HNRH3_HUMAN | HNRNPH3 | 36.9 | 3.6784 |
| 3 | 3 | sp\|Q9H9B1\|EHMT1_HUMAN | EHMT1 | 141.38 | 3.6655 |
| 3 | 3 | sp\|Q6PD62\|CTR9_HUMAN | CTR9 | 133.42 | 3.5951 |
| 3 | 3 | sp\|O15213\|WDR46_HUMAN | WDR46 | 68.03 | 3.5419 |
| 3 | 3 | sp\|Q9NQ55\|SSF1_HUMAN | PPAN | 53.16 | 3.5364 |
| 3 | 3 | sp\|Q9GZR7\|DDX24_HUMAN | DDX24 | 96.27 | 3.5302 |
| 3 | 3 | sp\|Q9BVI4\|NOC4L_HUMAN | NOC4L | 58.43 | 3.5196 |
| 3 | 3 | sp\|P49411\|EFTU_HUMAN | TUFM | 49.51 | 3.4818 |
| 3 | 3 | tr\|F8VXC8\|F8VXC8_HUMAN | SMARCC2 | 136.1 | 3.4615 |
| 3 | 3 | sp\|Q13330\|MTA1_HUMAN | MTA1 | 80.74 | 3.4299 |
| 3 | 3 | sp\|Q8TDI0\|CHD5_HUMAN | CHD5 | 222.91 | 3.4258 |
| 3 | 3 | sp\|P02545\|LMNA_HUMAN | LMNA | 74.09 | 3.4069 |
| 3 | 3 | tr\|A0A1P0AZG4\|A0A1P0AZG4_HUMAN | LCOR | 137.14 | 3.3907 |
| 3 | 3 | sp\|Q8IZL8\|PELP1_HUMAN | PELP1 | 119.62 | 3.3676 |
| 3 | 3 | sp\|Q9BQE9\|BCL7B_HUMAN | BCL7B | 22.18 | 3.2712 |
| 3 | 3 | sp\|P68104\|EF1A1_HUMAN | EEF1A1 | 50.11 | 3.2285 |
| 3 | 3 | sp\|Q9UMS4\|PRP19_HUMAN | PRPF19 | 55.15 | 3.2237 |
| 3 | 3 | sp\|P14618\|KPYM_HUMAN | PKM | 57.9 | 3.2008 |
| 3 | 3 | sp\|P56537\|IF6_HUMAN | EIF6 | 26.58 | 3.1738 |
| 3 | 3 | sp\|P04406\|G3P_HUMAN | GAPDH | 36.03 | 3.1734 |
| 3 | 3 | sp\|Q9NRL2\|BAZ1A_HUMAN | BAZ1A | 178.59 | 3.1621 |
| 3 | 3 | sp\|Q96G21\|IMP4_HUMAN | IMP4 | 33.74 | 3.1542 |
| 3 | 3 | sp\|P78316\|NOP14_HUMAN | NOP14 | 97.61 | 3.0703 |
| 3 | 3 | sp\|Q9H0D6\|XRN2_HUMAN | XRN2 | 108.51 | 3.0665 |
| 3 | 3 | sp\|P56182\|RRP1_HUMAN | RRP1 | 52.81 | 3.0433 |
| 3 | 3 | sp\|Q15050\|RRS1_HUMAN | RRS1 | 41.17 | 2.9918 |
| 3 | 3 | sp\|Q9H8H2\|DDX31_HUMAN | DDX31 | 94.03 | 2.9841 |
| 3 | 3 | sp\|P42285\|SK2L2_HUMAN | SKIV2L2 | 117.73 | 2.9221 |
| 3 | 3 | sp\|P09874\|PARP1_HUMAN | PARP1 | 113.01 | 2.9162 |
| 3 | 3 | sp\|Q99848\|EBP2_HUMAN | EBNA1BP2 | 34.83 | 2.9109 |
| 3 | 3 | sp\|Q96T37\|RBM15_HUMAN | RBM15 | 107.12 | 2.894 |
| 3 | 3 | sp\|P32119\|PRDX2_HUMAN | PRDX2 | 21.88 | 2.8741 |
| 3 | 3 | sp\|O95793\|STAU1_HUMAN | STAU1 | 63.14 | 2.8703 |
| 3 | 3 | sp\|Q9UGU0\|TCF20_HUMAN | TCF20 | 211.64 | 2.8664 |
| 3 | 3 | sp\|P62750\|RL23A_HUMAN | RPL23A | 17.68 | 2.8052 |
| 3 | 3 | sp\|P42167\|LAP2B_HUMAN | TMPO | 50.64 | 2.7995 |
| 3 | 3 | sp\|P61313\|RL15_HUMAN | RPL15 | 24.13 | 2.793 |
| 3 | 3 | sp\|P23396\|RS3_HUMAN | RPS3 | 26.67 | 2.7615 |
| 3 | 3 | sp\|Q6P0N0\|M18BP_HUMAN | MIS18BP1 | 129.01 | 2.7492 |
| 3 | 3 | sp\|Q53HL2\|BOREA_HUMAN | CDCA8 | 31.3 | 2.7294 |
| 3 | 3 | sp\|Q8N9T8\|KRI1_HUMAN | KRI1 | 82.55 | 2.7263 |
| 3 | 3 | sp\|P38432\|COIL_HUMAN | COIL | 62.57 | 2.6522 |
| 3 | 3 | sp\|Q5JTV8\|TOIP1_HUMAN | TOR1AIP1 | 66.21 | 2.5133 |
| 3 | 3 | sp\|P52292\|IMA1_HUMAN | KPNA2 | 57.83 | 2.5042 |
| 3 | 3 | sp\|Q9NZM5\|GSCR2_HUMAN | GLTSCR2 | 54.36 | 2.4664 |
| 3 | 3 | sp\|Q9HC52\|CBX8_HUMAN | CBX8 | 43.37 | 2.4195 |
| 3 | 3 | sp\|P62851\|RS25_HUMAN | RPS25 | 13.73 | 2.387 |
| 3 | 3 | sp\|Q8NI36\|WDR36_HUMAN | WDR36 | 105.26 | 2.3862 |
| 3 | 3 | sp\|Q02878\|RL6_HUMAN | RPL6 | 32.71 | 2.3594 |
| 3 | 3 | sp\|P46781\|RS9_HUMAN | RPS9 | 22.58 | 2.3229 |
| 3 | 3 | sp\|Q9NVI7\|ATD3A_HUMAN | ATAD3A | 71.32 | 2.1869 |
| 2 | 3 | sp\|Q14103\|HNRPD_HUMAN | HNRNPD | 38.41 | 3.6101 |
| 2 | 3 | sp\|Q02539\|H11_HUMAN | HIST1H1A | 21.83 | 2.8548 |
| 2 | 3 | sp\|Q71UI9\|H2AV_HUMAN | H2AFV | 13.5 | 2.7277 |
| 2 | 3 | sp\|Q13610\|PWP1_HUMAN | PWP1 | 55.79 | 2.6004 |
| 2 | 3 | sp\|O15226\|NKRF_HUMAN | NKRF | 77.62 | 2.3415 |
| 2 | 3 | sp\|P84103\|SRSF3_HUMAN | SRSF3 | 19.32 | 2.2047 |
| 1 | 3 | sp\|P07305\|H10_HUMAN | H1F0 | 20.85 | 3.9925 |
| 1 | 3 | sp\|P16104\|H2AX_HUMAN | H2AFX | 15.14 | 3.8965 |
| 2 | 2 | sp\|Q9NS69\|TOM22_HUMAN | TOMM22 | 15.51 | 5.0427 |
| 2 | 2 | sp\|P32455\|GBP1_HUMAN | GBP1 | 67.89 | 4.7845 |
| 2 | 2 | sp\|Q14498\|RBM39_HUMAN | RBM39 | 59.34 | 4.5672 |
| 2 | 2 | sp\|P78364\|PHC1_HUMAN | PHC1 | 105.47 | 4.4038 |
| 2 | 2 | sp\|P62241\|RS8_HUMAN | RPS8 | 24.19 | 4.3331 |
| 2 | 2 | sp\|Q96EU6\|RRP36_HUMAN | RRP36 | 29.8 | 4.2929 |
| 2 | 2 | sp\|P83916\|CBX1_HUMAN | CBX1 | 21.4 | 4.1311 |
| 2 | 2 | sp\|O14983\|AT2A1_HUMAN | ATP2A1 | 110.18 | 4.1184 |
| 2 | 2 | sp\|P35580\|MYH10_HUMAN | MYH10 | 228.86 | 4.1122 |
| 2 | 2 | sp\|P49711\|CTCF_HUMAN | CTCF | 82.73 | 4.0754 |
| 2 | 2 | sp\|O95251\|KAT7_HUMAN | KAT7 | 70.6 | 4.0575 |
| 2 | 2 | sp\|P35453\|HXD13_HUMAN | HOXD13 | 36.08 | 4.0572 |
| 2 | 2 | sp\|Q07020\|RL18_HUMAN | RPL18 | 21.62 | 4.0568 |
| 2 | 2 | sp\|Q92769\|HDAC2_HUMAN | HDAC2 | 55.33 | 4.028 |
| 2 | 2 | sp\|Q9H7B2\|RPF2_HUMAN | RPF2 | 35.56 | 4.0267 |
| 2 | 2 | sp\|Q92784\|DPF3_HUMAN | DPF3 | 43.06 | 3.9575 |
| 2 | 2 | sp\|Q8WVM7\|STAG1_HUMAN | STAG1 | 144.34 | 3.8979 |
| 2 | 2 | sp\|Q9H4L4\|SENP3_HUMAN | SENP3 | 64.97 | 3.8924 |
| 2 | 2 | sp\|P39023\|RL3_HUMAN | RPL3 | 46.08 | 3.7618 |
| 2 | 2 | sp\|P67809\|YBOX1_HUMAN | YBX1 | 35.9 | 3.751 |
| 2 | 2 | sp\|Q8NEJ9\|NGDN_HUMAN | NGDN | 35.87 | 3.7496 |
| 2 | 2 | sp\|Q13111\|CAF1A_HUMAN | CHAF1A | 106.86 | 3.7075 |
| 2 | 2 | sp\|O00267\|SPT5H_HUMAN | SUPT5H | 120.92 | 3.697 |
| 2 | 2 | sp\|Q6DKI1\|RL7L_HUMAN | RPL7L1 | 28.64 | 3.6794 |
| 2 | 2 | sp\|Q5VZL5\|ZMYM4_HUMAN | ZMYM4 | 172.68 | 3.6656 |
| 2 | 2 | sp\|Q9H7H0\|MET17_HUMAN | METTL17 | 50.7 | 3.6536 |
| 2 | 2 | sp\|O95983\|MBD3_HUMAN | MBD3 | 32.82 | 3.6402 |
| 2 | 2 | sp\|P32969\|RL9_HUMAN | RPL9 | 21.85 | 3.6356 |
| 2 | 2 | tr\|B2RC06\|B2RC06_HUMAN | | 39.25 | 3.6326 |
| 2 | 2 | sp\|Q92522\|H1X_HUMAN | H1FX | 22.47 | 3.6307 |
| 2 | 2 | sp\|P04792\|HSPB1_HUMAN | HSPB1 | 22.77 | 3.6296 |
| 2 | 2 | sp\|P05141\|ADT2_HUMAN | SLC25A5 | 32.83 | 3.6195 |
| 2 | 2 | sp\|P63244\|RACK1_HUMAN | RACK1 | 35.05 | 3.6189 |
| 2 | 2 | sp\|P04040\|CATA_HUMAN | CAT | 59.72 | 3.5759 |
| 2 | 2 | sp\|Q8WXF0\|SRS12_HUMAN | SRSF12 | 30.49 | 3.5688 |
| 2 | 2 | sp\|Q9Y6K1\|DNM3A_HUMAN | DNMT3A | 101.79 | 3.5118 |
| 2 | 2 | sp\|Q86WX3\|AROS_HUMAN | RPS19BP1 | 15.42 | 3.5097 |
| 2 | 2 | sp\|Q96GD4\|AURKB_HUMAN | AURKB | 39.29 | 3.4963 |
| 2 | 2 | sp\|Q96HW7\|INT4_HUMAN | INTS4 | 108.1 | 3.3641 |
| 2 | 2 | sp\|Q9NSI2\|F207A_HUMAN | FAM207A | 25.44 | 3.3636 |
| 2 | 2 | sp\|O75494\|SRS10_HUMAN | SRSF10 | 31.28 | 3.3457 |
| 2 | 2 | sp\|Q9NW13\|RBM28_HUMAN | RBM28 | 85.68 | 3.3224 |
| 2 | 2 | sp\|P62906\|RL10A_HUMAN | RPL10A | 24.82 | 3.319 |
| 2 | 2 | sp\|Q13123\|RED_HUMAN | IK | 65.56 | 3.3104 |
| 2 | 2 | sp\|Q8WUM0\|NU133_HUMAN | NUP133 | 128.9 | 3.3062 |
| 2 | 2 | sp\|P14678\|RSMB_HUMAN | SNRPB | 24.59 | 3.3027 |
| 2 | 2 | sp\|Q15517\|CDSN_HUMAN | CDSN | 51.49 | 3.2564 |
| 2 | 2 | sp\|P17661\|DESM_HUMAN | DES | 53.5 | 3.1482 |
| 2 | 2 | sp\|Q8NC56\|LEMD2_HUMAN | LEMD2 | 56.94 | 3.1402 |
| 2 | 2 | sp\|Q9UNQ2\|DIM1_HUMAN | DIMT1 | 35.21 | 3.1391 |
| 2 | 2 | sp\|Q00325\|MPCP_HUMAN | SLC25A3 | 40.07 | 3.1288 |
| 2 | 2 | sp\|Q9NP55\|BPIA1_HUMAN | BPIFA1 | 26.7 | 3.1215 |
| 2 | 2 | sp\|Q9BZJ0\|CRNL1_HUMAN | CRNKL1 | 100.39 | 3.1064 |
| 2 | 2 | sp\|Q9NV31\|IMP3_HUMAN | IMP3 | 21.84 | 3.0688 |
| 2 | 2 | sp\|O15523\|DDX3Y_HUMAN | DDX3Y | 73.11 | 3.0673 |
| 2 | 2 | sp\|Q53GS7\|GLE1_HUMAN | GLE1 | 79.79 | 3.0633 |
| 2 | 2 | sp\|Q15717\|ELAV1_HUMAN | ELAVL1 | 36.07 | 3.0494 |
| 2 | 2 | sp\|Q9BQF6\|SENP7_HUMAN | SENP7 | 119.58 | 3.0117 |
| 2 | 2 | sp\|P83731\|RL24_HUMAN | RPL24 | 17.77 | 3.0103 |
| 2 | 2 | sp\|P08574\|CY1_HUMAN | CYC1 | 35.4 | 2.999 |
| 2 | 2 | sp\|Q7KZ85\|SPT6H_HUMAN | SUPT6H | 198.95 | 2.9924 |
| 2 | 2 | sp\|P30050\|RL12_HUMAN | RPL12 | 17.81 | 2.9874 |
| 2 | 2 | sp\|Q15059\|BRD3_HUMAN | BRD3 | 79.49 | 2.943 |
| 2 | 2 | sp\|P62263\|RS14_HUMAN | RPS14 | 16.26 | 2.9348 |
| 2 | 2 | sp\|Q9Y6C9\|MTCH2_HUMAN | MTCH2 | 33.31 | 2.925 |
| 2 | 2 | sp\|P38919\|IF4A3_HUMAN | EIF4A3 | 46.84 | 2.9081 |
| 2 | 2 | sp\|Q8TAE8\|G45IP_HUMAN | GADD45GIP1 | 25.37 | 2.8833 |
| 2 | 2 | sp\|Q03252\|LMNB2_HUMAN | LMNB2 | 69.91 | 2.8508 |
| 2 | 2 | sp\|P22695\|QCR2_HUMAN | UQCRC2 | 48.41 | 2.8347 |
| 2 | 2 | sp\|Q5TAP6\|UT14C_HUMAN | UTP14C | 87.13 | 2.8136 |
| 2 | 2 | sp\|Q13595\|TRA2A_HUMAN | TRA2A | 32.67 | 2.7892 |
| 2 | 2 | sp\|P10599\|THIO_HUMAN | TXN | 11.73 | 2.7799 |
| 2 | 2 | sp\|Q9UHA3\|RLP24_HUMAN | RSL24D1 | 19.61 | 2.7718 |
| 2 | 2 | sp\|Q8N0S6\|CENPL_HUMAN | CENPL | 38.97 | 2.7494 |
| 2 | 2 | sp\|Q14781\|CBX2_HUMAN | CBX2 | 56.05 | 2.7332 |
| 2 | 2 | sp\|Q15910\|EZH2_HUMAN | EZH2 | 85.31 | 2.7153 |
| 2 | 2 | sp\|P62304\|RUXE_HUMAN | SNRPE | 10.8 | 2.7029 |
| 2 | 2 | sp\|Q99590\|SCAFB_HUMAN | SCAF11 | 164.55 | 2.6846 |
| 2 | 2 | sp\|P52746\|ZN142_HUMAN | ZNF142 | 187.76 | 2.636 |
| 2 | 2 | sp\|Q96L73\|NSD1_HUMAN | NSD1 | 296.46 | 2.6321 |
| 2 | 2 | sp\|Q15149\|PLEC_HUMAN | PLEC | 531.47 | 2.6286 |
| 2 | 2 | sp\|P05023\|AT1A1_HUMAN | ATP1A1 | 112.82 | 2.5941 |
| 2 | 2 | sp\|P26373\|RL13_HUMAN | RPL13 | 24.25 | 2.5795 |
| 2 | 2 | sp\|P52948\|NUP98_HUMAN | NUP98 | 197.46 | 2.5777 |
| 2 | 2 | sp\|Q9NW64\|RBM22_HUMAN | RBM22 | 46.87 | 2.521 |
| 2 | 2 | sp\|O14979\|HNRDL_HUMAN | HNRNPDL | 46.41 | 2.5206 |
| 2 | 2 | sp\|Q96HS1\|PGAM5_HUMAN | PGAM5 | 31.98 | 2.5018 |
| 2 | 2 | sp\|P62249\|RS16_HUMAN | RPS16 | 16.44 | 2.3822 |
| 2 | 2 | sp\|Q14966\|ZN638_HUMAN | ZNF638 | 220.49 | 2.3648 |
| 2 | 2 | sp\|Q9NRG9\|AAAS_HUMAN | AAAS | 59.54 | 2.3486 |
| 2 | 2 | sp\|Q86U38\|NOP9_HUMAN | NOP9 | 69.39 | 2.3397 |
| 2 | 2 | sp\|P25311\|ZA2G_HUMAN | AZGP1 | 34.24 | 2.2809 |
| 2 | 2 | sp\|Q13895\|BYST_HUMAN | BYSL | 49.57 | 2.2751 |
| 1 | 2 | sp\|Q01130\|SRSF2_HUMAN | SRSF2 | 25.46 | 5.126 |
| 1 | 2 | tr\|A0A0M3HER2\|A0A0M3HER2_HUMAN | CENPV | 18.8 | 4.1321 |
| 1 | 2 | sp\|O75151\|PHF2_HUMAN | PHF2 | 120.7 | 3.6742 |
| 1 | 2 | sp\|O75323\|NIPS2_HUMAN | NIPSNAP2 | 33.72 | 2.0829 |
| 1 | 1 | sp\|P08708\|RS17_HUMAN | RPS17 | 15.54 | 5.521 |
| 1 | 1 | sp\|P08865\|RSSA_HUMAN | RPSA | 32.83 | 5.1704 |
| 1 | 1 | sp\|P07355\|ANXA2_HUMAN | ANXA2 | 38.58 | 5.1093 |
| 1 | 1 | sp\|Q6PK04\|CC137_HUMAN | CCDC137 | 33.21 | 5.0221 |
| 1 | 1 | sp\|O94906\|PRP6_HUMAN | PRPF6 | 106.86 | 4.9042 |
| 1 | 1 | sp\|Q3ZCQ8\|TIM50_HUMAN | TIMM50 | 39.62 | 4.8852 |
| 1 | 1 | tr\|A8K7N0\|A8K7N0_HUMAN | | 23.63 | 4.8498 |
| 1 | 1 | tr\|A0A0A0MQS2\|A0A0A0MQS2_HUMAN | CLASRP | 77.12 | 4.7872 |
| 1 | 1 | sp\|Q9NQZ2\|SAS10_HUMAN | UTP3 | 54.53 | 4.7178 |
| 1 | 1 | sp\|Q13243\|SRSF5_HUMAN | SRSF5 | 31.25 | 4.7078 |
| 1 | 1 | sp\|Q96MU7\|YTDC1_HUMAN | YTHDC1 | 84.65 | 4.7045 |
| 1 | 1 | tr\|Q6IPH7\|Q6IPH7_HUMAN | RPL14 | 23.77 | 4.6788 |
| 1 | 1 | tr\|S4R341\|S4R341_HUMAN | NOLC1 | 8.05 | 4.663 |
| 1 | 1 | sp\|O43159\|RRP8_HUMAN | RRP8 | 50.68 | 4.6332 |
| 1 | 1 | sp\|P50914\|RL14_HUMAN | RPL14 | 23.42 | 4.618 |
| 1 | 1 | sp\|Q9GZL7\|WDR12_HUMAN | WDR12 | 47.68 | 4.5869 |
| 1 | 1 | sp\|Q9GZR2\|REXO4_HUMAN | REXO4 | 46.64 | 4.5713 |
| 1 | 1 | sp\|Q8NAF0\|ZN579_HUMAN | ZNF579 | 60.47 | 4.5123 |
| 1 | 1 | sp\|P62913\|RL11_HUMAN | RPL11 | 20.24 | 4.4569 |
| 1 | 1 | sp\|P16989\|YBOX3_HUMAN | YBX3 | 40.07 | 4.4487 |
| 1 | 1 | sp\|Q75QN2\|INT8_HUMAN | INTS8 | 113.02 | 4.428 |
| 1 | 1 | sp\|Q13263\|TIF1B_HUMAN | TRIM28 | 88.49 | 4.4118 |
| 1 | 1 | sp\|Q8WYB5\|KAT6B_HUMAN | KAT6B | 231.23 | 4.3886 |
| 1 | 1 | tr\|Q9UL78\|Q9UL78_HUMAN | | 11.64 | 4.3545 |
| 1 | 1 | sp\|O94880\|PHF14_HUMAN | PHF14 | 99.99 | 4.3129 |
| 1 | 1 | sp\|Q7Z4V5\|HDGR2_HUMAN | HDGFL2 | 74.27 | 4.3001 |
| 1 | 1 | sp\|Q13867\|BLMH_HUMAN | BLMH | 52.53 | 4.268 |
| 1 | 1 | sp\|Q86Y91\|KI18B_HUMAN | KIF18B | 94.16 | 4.2378 |
| 1 | 1 | sp\|Q32P51\|RA1L2_HUMAN | HNRNPA1L2 | 34.2 | 4.2125 |
| 1 | 1 | sp\|P55081\|MFAP1_HUMAN | MFAP1 | 51.93 | 4.1959 |
| 1 | 1 | sp\|Q96SK2\|TM209_HUMAN | TMEM209 | 62.88 | 4.194 |
| 1 | 1 | sp\|P62829\|RL23_HUMAN | RPL23 | 14.86 | 4.1813 |
| 1 | 1 | sp\|Q9NQ39\|RS10L_HUMAN | RPS10P5 | 20.11 | 4.1778 |
| 1 | 1 | tr\|B2RWN5\|B2RWN5_HUMAN | HEATR1 | 242.11 | 4.1764 |
| 1 | 1 | sp\|Q9HCM4\|E41L5_HUMAN | EPB41L5 | 81.8 | 4.1232 |
| 1 | 1 | sp\|P26599\|PTBP1_HUMAN | PTBP1 | 57.19 | 4.1098 |
| 1 | 1 | sp\|Q96EY7\|PTCD3_HUMAN | PTCD3 | 78.5 | 4.0773 |
| 1 | 1 | sp\|P20226\|TBP_HUMAN | TBP | 37.67 | 4.068 |
| 1 | 1 | tr\|B7Z8Y3\|B7Z8Y3_HUMAN | | 106.95 | 4.036 |
| 1 | 1 | sp\|P35658\|NU214_HUMAN | NUP214 | 213.49 | 4.017 |
| 1 | 1 | sp\|Q9UGM3\|DMBT1_HUMAN | DMBT1 | 260.57 | 4.0093 |
| 1 | 1 | sp\|Q9BW27\|NUP85_HUMAN | NUP85 | 74.97 | 4.0026 |
| 1 | 1 | sp\|O15381\|NVL_HUMAN | NVL | 94.99 | 3.9636 |
| 1 | 1 | sp\|Q14974\|IMB1_HUMAN | KPNB1 | 97.11 | 3.9488 |
| 1 | 1 | sp\|P0DMV9\|HS71B_HUMAN | HSPA1B | 70.01 | 3.9319 |
| 1 | 1 | sp\|Q76FK4\|NOL8_HUMAN | NOL8 | 131.54 | 3.9233 |
| 1 | 1 | sp\|O60508\|PRP17_HUMAN | CDC40 | 65.48 | 3.9111 |
| 1 | 1 | sp\|P09234\|RU1C_HUMAN | SNRPC | 17.38 | 3.8968 |
| 1 | 1 | sp\|Q9NRZ9\|HELLS_HUMAN | HELLS | 97.01 | 3.8903 |
| 1 | 1 | sp\|Q9Y3A2\|UTP11_HUMAN | UTP11 | 30.43 | 3.8848 |
| 1 | 1 | sp\|Q13415\|ORC1_HUMAN | ORC1 | 97.29 | 3.8764 |
| 1 | 1 | sp\|Q69YH5\|CDCA2_HUMAN | CDCA2 | 112.61 | 3.8703 |
| 1 | 1 | sp\|Q6IQ32\|ADNP2_HUMAN | ADNP2 | 122.75 | 3.8567 |
| 1 | 1 | sp\|O43795\|MYO1B_HUMAN | MYO1B | 131.9 | 3.833 |
| 1 | 1 | sp\|Q9P035\|HACD3_HUMAN | HACD3 | 43.13 | 3.8266 |
| 1 | 1 | sp\|Q9BYN8\|RT26_HUMAN | MRPS26 | 24.2 | 3.8217 |
| 1 | 1 | sp\|P07437\|TBB5_HUMAN | TUBB | 49.64 | 3.818 |
| 1 | 1 | sp\|O15042\|SR140_HUMAN | U2SURP | 118.22 | 3.8052 |
| 1 | 1 | sp\|Q01831\|XPC_HUMAN | XPC | 105.89 | 3.8043 |
| 1 | 1 | tr\|B0UZZ8\|B0UZZ8_HUMAN | C6orf11 | 68 | 3.7793 |
| 1 | 1 | sp\|P13929\|ENOB_HUMAN | ENO3 | 46.96 | 3.7735 |
| 1 | 1 | sp\|Q9BWN1\|PRR14_HUMAN | PRR14 | 64.29 | 3.7614 |
| 1 | 1 | sp\|Q9UIS9\|MBD1_HUMAN | MBD1 | 66.56 | 3.7254 |
| 1 | 1 | sp\|Q9BYD2\|RM09_HUMAN | MRPL9 | 30.22 | 3.7014 |
| 1 | 1 | sp\|Q9NPI1\|BRD7_HUMAN | BRD7 | 74.09 | 3.6873 |
| 1 | 1 | sp\|Q9BYD3\|RM04_HUMAN | MRPL4 | 34.9 | 3.6809 |
| 1 | 1 | sp\|P42696\|RBM34_HUMAN | RBM34 | 48.54 | 3.6619 |
| 1 | 1 | sp\|Q13206\|DDX10_HUMAN | DDX10 | 100.83 | 3.6542 |
| 1 | 1 | sp\|Q07021\|C1QBP_HUMAN | C1QBP | 31.34 | 3.6263 |
| 1 | 1 | sp\|Q8WVC0\|LEO1_HUMAN | LEO1 | 75.36 | 3.6184 |
| 1 | 1 | sp\|Q69YN4\|VIR_HUMAN | KIAA1429 | 201.9 | 3.6178 |
| 1 | 1 | sp\|P12277\|KCRB_HUMAN | CKB | 42.62 | 3.5894 |
| 1 | 1 | sp\|Q9Y3B9\|RRP15_HUMAN | RRP15 | 31.46 | 3.5847 |
| 1 | 1 | sp\|O43251\|RFOX2_HUMAN | RBFOX2 | 41.35 | 3.5765 |
| 1 | 1 | sp\|Q8N1G0\|ZN687_HUMAN | ZNF687 | 129.45 | 3.5753 |
| 1 | 1 | sp\|Q14669\|TRIPC_HUMAN | TRIP12 | 220.3 | 3.5724 |
| 1 | 1 | sp\|P10523\|ARRS_HUMAN | SAG | 45.09 | 3.563 |
| 1 | 1 | sp\|Q9HAF1\|EAF6_HUMAN | MEAF6 | 21.62 | 3.5254 |
| 1 | 1 | sp\|P01834\|IGKC_HUMAN | IGKC | 11.76 | 3.5209 |
| 1 | 1 | sp\|Q5T280\|CI114_HUMAN | SPOUT1 | 41.98 | 3.519 |
| 1 | 1 | sp\|P38646\|GRP75_HUMAN | HSPA9 | 73.63 | 3.5173 |
| 1 | 1 | sp\|Q9H6R0\|DHX33_HUMAN | DHX33 | 78.82 | 3.5069 |
| 1 | 1 | sp\|O00571\|DDX3X_HUMAN | DDX3X | 73.2 | 3.5066 |
| 1 | 1 | sp\|Q06587\|RING1_HUMAN | RING1 | 42.4 | 3.4787 |
| 1 | 1 | sp\|Q13151\|ROA0_HUMAN | HNRNPA0 | 30.82 | 3.4486 |
| 1 | 1 | sp\|O75934\|SPF27_HUMAN | BCAS2 | 26.11 | 3.4476 |
| 1 | 1 | sp\|Q9UBB5\|MBD2_HUMAN | MBD2 | 43.23 | 3.4443 |
| 1 | 1 | sp\|Q9NX63\|MIC19_HUMAN | CHCHD3 | 26.14 | 3.4382 |
| 1 | 1 | tr\|Q05CW7\|Q05CW7_HUMAN | NAT10 | 62.35 | 3.3784 |
| 1 | 1 | sp\|P49759\|CLK1_HUMAN | CLK1 | 57.25 | 3.3643 |
| 1 | 1 | sp\|Q58FF8\|H90B2_HUMAN | HSP90AB2P | 44.32 | 3.358 |
| 1 | 1 | sp\|P62258\|1433E_HUMAN | YWHAE | 29.16 | 3.3441 |
| 1 | 1 | sp\|Q15365\|PCBP1_HUMAN | PCBP1 | 37.47 | 3.3429 |
| 1 | 1 | sp\|Q13148\|TADBP_HUMAN | TARDBP | 44.71 | 3.3393 |
| 1 | 1 | sp\|P62847\|RS24_HUMAN | RPS24 | 15.41 | 3.2951 |
| 1 | 1 | sp\|P41219\|PERI_HUMAN | PRPH | 53.62 | 3.2937 |
| 1 | 1 | sp\|O00148\|DX39A_HUMAN | DDX39A | 49.1 | 3.2936 |
| 1 | 1 | sp\|P12236\|ADT3_HUMAN | SLC25A6 | 32.85 | 3.2759 |
| 1 | 1 | sp\|Q8NHW5\|RLA0L_HUMAN | RPLP0P6 | 34.34 | 3.2647 |
| 1 | 1 | sp\|O60832\|DKC1_HUMAN | DKC1 | 57.64 | 3.2545 |
| 1 | 1 | sp\|Q13129\|RLF_HUMAN | RLF | 217.81 | 3.2327 |
| 1 | 1 | sp\|Q02241\|KIF23_HUMAN | KIF23 | 109.99 | 3.2283 |
| 1 | 1 | sp\|P26368\|U2AF2_HUMAN | U2AF2 | 53.47 | 3.2053 |
| 1 | 1 | sp\|Q9ULW3\|ABT1_HUMAN | ABT1 | 31.06 | 3.2034 |
| 1 | 1 | KV2A7_MOUSE | | 12.27 | 3.1941 |
| 1 | 1 | sp\|P21333\|FLNA_HUMAN | FLNA | 280.56 | 3.1928 |
| 1 | 1 | sp\|Q8TF76\|HASP_HUMAN | GSG2 | 88.44 | 3.1876 |
| 1 | 1 | sp\|Q92665\|RT31_HUMAN | MRPS31 | 45.29 | 3.1529 |
| 1 | 1 | sp\|P04843\|RPN1_HUMAN | RPN1 | 68.53 | 3.1371 |
| 1 | 1 | sp\|O75223\|GGCT_HUMAN | GGCT | 20.99 | 3.1294 |
| 1 | 1 | sp\|P40939\|ECHA_HUMAN | HADHA | 82.95 | 3.0859 |
| 1 | 1 | sp\|E9PRG8\|CK098_HUMAN | C11orf98 | 13.79 | 3.0795 |
| 1 | 1 | sp\|Q9BXF3\|CECR2_HUMAN | CECR2 | 164.11 | 3.075 |
| 1 | 1 | sp\|Q8IXM6\|NRM_HUMAN | NRM | 29.36 | 3.0489 |
| 1 | 1 | sp\|O94901\|SUN1_HUMAN | SUN1 | 90.01 | 3.041 |
| 1 | 1 | sp\|P01876\|IGHA1_HUMAN | IGHA1 | 37.63 | 3.0351 |
| 1 | 1 | tr\|A0A024R383\|A0A024R383_HUMAN | hCG_21098 | 172.58 | 3.0327 |
| 1 | 1 | sp\|O94805\|ACL6B_HUMAN | ACTL6B | 46.85 | 3.0307 |
| 1 | 1 | sp\|Q96EP5\|DAZP1_HUMAN | DAZAP1 | 43.36 | 3.0232 |
| 1 | 1 | tr\|Q53F64\|Q53F64_HUMAN | | 35.97 | 3.005 |
| 1 | 1 | sp\|P0DOX7\|IGK_HUMAN | | 23.36 | 2.9832 |
| 1 | 1 | sp\|P34931\|HS71L_HUMAN | HSPA1L | 70.33 | 2.971 |
| 1 | 1 | sp\|Q7Z5J4\|RAI1_HUMAN | RAI1 | 203.23 | 2.9686 |
| 1 | 1 | sp\|Q9Y6A4\|CFA20_HUMAN | CFAP20 | 22.76 | 2.9642 |
| 1 | 1 | sp\|P43246\|MSH2_HUMAN | MSH2 | 104.68 | 2.9358 |
| 1 | 1 | sp\|Q9NQ50\|RM40_HUMAN | MRPL40 | 24.48 | 2.9286 |
| 1 | 1 | sp\|Q8TEM1\|PO210_HUMAN | NUP210 | 204.98 | 2.9195 |
| 1 | 1 | sp\|Q09161\|NCBP1_HUMAN | NCBP1 | 91.78 | 2.9158 |
| 1 | 1 | sp\|Q13242\|SRSF9_HUMAN | SRSF9 | 25.53 | 2.9054 |
| 1 | 1 | sp\|Q9Y2R9\|RT07_HUMAN | MRPS7 | 28.12 | 2.8919 |
| 1 | 1 | sp\|Q9NY12\|GAR1_HUMAN | GAR1 | 22.33 | 2.8874 |
| 1 | 1 | sp\|Q9UQ88\|CD11A_HUMAN | CDK11A | 91.31 | 2.8526 |
| 1 | 1 | sp\|Q8N6I1\|EID2_HUMAN | EID2 | 25.17 | 2.8514 |
| 1 | 1 | sp\|Q9UNL2\|SSRG_HUMAN | SSR3 | 21.07 | 2.8211 |
| 1 | 1 | sp\|O75530\|EED_HUMAN | EED | 50.17 | 2.8165 |
| 1 | 1 | sp\|P47914\|RL29_HUMAN | RPL29 | 17.74 | 2.801 |
| 1 | 1 | sp\|Q12830\|BPTF_HUMAN | BPTF | 338.05 | 2.7991 |
| 1 | 1 | sp\|Q8IWT3\|CUL9_HUMAN | CUL9 | 281.05 | 2.7852 |
| 1 | 1 | sp\|O95232\|LC7L3_HUMAN | LUC7L3 | 51.44 | 2.7789 |
| 1 | 1 | sp\|P0DMR1\|HNRC4_HUMAN | HNRNPCL4 | 32.01 | 2.7683 |
| 1 | 1 | sp\|Q5T3J3\|LRIF1_HUMAN | LRIF1 | 84.52 | 2.7663 |
| 1 | 1 | sp\|P01857\|IGHG1_HUMAN | IGHG1 | 36.08 | 2.7607 |
| 1 | 1 | sp\|P51571\|SSRD_HUMAN | SSR4 | 18.99 | 2.7559 |
| 1 | 1 | sp\|P46783\|RS10_HUMAN | RPS10 | 18.89 | 2.7434 |
| 1 | 1 | sp\|Q01469\|FABP5_HUMAN | FABP5 | 15.15 | 2.6709 |
| 1 | 1 | sp\|O60506\|HNRPQ_HUMAN | SYNCRIP | 69.56 | 2.6267 |
| 1 | 1 | sp\|P68871\|HBB_HUMAN | HBB | 15.99 | 2.6157 |
| 1 | 1 | sp\|Q5H9F3\|BCORL_HUMAN | BCORL1 | 182.41 | 2.6122 |
| 1 | 1 | sp\|Q9Y232\|CDYL1_HUMAN | CDYL | 66.44 | 2.6118 |
| 1 | 1 | sp\|Q09028\|RBBP4_HUMAN | RBBP4 | 47.63 | 2.6044 |
| 1 | 1 | sp\|Q01844\|EWS_HUMAN | EWSR1 | 68.44 | 2.603 |
| 1 | 1 | sp\|Q9NWU5\|RM22_HUMAN | MRPL22 | 23.63 | 2.5935 |
| 1 | 1 | sp\|A8MTJ3\|GNAT3_HUMAN | GNAT3 | 40.33 | 2.5839 |
| 1 | 1 | sp\|O14647\|CHD2_HUMAN | CHD2 | 211.21 | 2.5688 |
| 1 | 1 | sp\|P56134\|ATPK_HUMAN | ATP5J2 | 10.91 | 2.5572 |
| 1 | 1 | sp\|Q9HBE1\|PATZ1_HUMAN | PATZ1 | 74.01 | 2.5483 |
| 1 | 1 | sp\|Q6DRA6\|H2B2D_HUMAN | HIST2H2BD | 18.01 | 2.5415 |
| 1 | 1 | sp\|P54652\|HSP72_HUMAN | HSPA2 | 69.98 | 2.5309 |
| 1 | 1 | sp\|Q9UKD2\|MRT4_HUMAN | MRTO4 | 27.54 | 2.5209 |
| 1 | 1 | sp\|Q8N201\|INT1_HUMAN | INTS1 | 244.14 | 2.5195 |
| 1 | 1 | sp\|Q5SRE5\|NU188_HUMAN | NUP188 | 195.92 | 2.4771 |
| 1 | 1 | sp\|Q9Y4F1\|FARP1_HUMAN | FARP1 | 118.56 | 2.4621 |
| 1 | 1 | sp\|P17010\|ZFX_HUMAN | ZFX | 90.46 | 2.439 |
| 1 | 1 | sp\|Q7Z2K6\|ERMP1_HUMAN | ERMP1 | 100.17 | 2.4223 |
| 1 | 1 | sp\|Q9UJZ1\|STML2_HUMAN | STOML2 | 38.51 | 2.4195 |
| 1 | 1 | sp\|P62269\|RS18_HUMAN | RPS18 | 17.71 | 2.417 |
| 1 | 1 | sp\|Q16352\|AINX_HUMAN | INA | 55.36 | 2.4143 |
| 1 | 1 | sp\|Q9BRL6\|SRSF8_HUMAN | SRSF8 | 32.27 | 2.3771 |
| 1 | 1 | sp\|Q9HC84\|MUC5B_HUMAN | MUC5B | 595.96 | 2.3769 |
| 1 | 1 | sp\|P11488\|GNAT1_HUMAN | GNAT1 | 40.02 | 2.3735 |
| 1 | 1 | sp\|Q9HCD5\|NCOA5_HUMAN | NCOA5 | 65.5 | 2.3666 |
| 1 | 1 | sp\|P43304\|GPDM_HUMAN | GPD2 | 80.8 | 2.3647 |
| 1 | 1 | sp\|Q15388\|TOM20_HUMAN | TOMM20 | 16.29 | 2.3634 |
| 1 | 1 | sp\|P61353\|RL27_HUMAN | RPL27 | 15.79 | 2.3498 |
| 1 | 1 | sp\|Q9BXY5\|CAYP2_HUMAN | CAPS2 | 63.8 | 2.3229 |
| 1 | 1 | sp\|Q9BZE1\|RM37_HUMAN | MRPL37 | 48.09 | 2.288 |
| 1 | 1 | sp\|Q9P2K5\|MYEF2_HUMAN | MYEF2 | 64.08 | 2.2862 |
| 1 | 1 | tr\|A0A024R5M9\|A0A024R5M9_HUMAN | NUMA1 | 236.37 | 2.2646 |
| 1 | 1 | sp\|Q06830\|PRDX1_HUMAN | PRDX1 | 22.1 | 2.2423 |
| 1 | 1 | sp\|Q9UI42\|CBPA4_HUMAN | CPA4 | 47.32 | 2.2271 |
| 1 | 1 | sp\|O75152\|ZC11A_HUMAN | ZC3H11A | 89.08 | 2.2039 |
| 1 | 1 | sp\|Q9NVX2\|NLE1_HUMAN | NLE1 | 53.29 | 2.1771 |
| 1 | 1 | sp\|Q14156\|EFR3A_HUMAN | EFR3A | 92.86 | 2.1643 |
| 1 | 1 | sp\|P22314\|UBA1_HUMAN | UBA1 | 117.77 | 2.1283 |

TABLE 5D
HA-SS18WT_NE_peptides

| Unique | Total | reference | Gene Symbol | MWT(kDa) | AVG |
|---|---|---|---|---|---|
| 55 | 595 | sp\|P51532\|SMCA4_HUMAN | SMARCA4 | 184.53 | 3.203 |
| 86 | 536 | sp\|P51531\|SMCA2_HUMAN | SMARCA2 | 181.17 | 3.0037 |
| 105 | 448 | sp\|O14497\|ARI1A_HUMAN | ARID1A | 241.89 | 3.1848 |
| 56 | 414 | sp\|Q92922\|SMRC1_HUMAN | SMARCC1 | 122.79 | 2.9463 |
| 69 | 368 | sp\|Q8TAQ2\|SMRC2_HUMAN | SMARCC2 | 132.8 | 2.9867 |
| 95 | 307 | sp\|Q8NFD5\|ARI1B_HUMAN | ARID1B | 235.97 | 2.9764 |
| 45 | 158 | sp\|Q9NZM4\|BICRA_HUMAN | BICRA | 158.39 | 3.144 |
| 29 | 157 | sp\|O96019\|ACL6A_HUMAN | ACTL6A | 47.43 | 3.1706 |
| 32 | 144 | sp\|Q96GM5\|SMRD1_HUMAN | SMARCD1 | 58.2 | 3.2163 |
| 31 | 137 | sp\|Q6STE5\|SMRD3_HUMAN | SMARCD3 | 54.98 | 3.1622 |
| 34 | 120 | sp\|Q969G3\|SMCE1_HUMAN | SMARCE1 | 46.62 | 3.2044 |
| 13 | 115 | sp\|P62736\|ACTA_HUMAN | ACTA2 | 41.98 | 2.6075 |
| 23 | 109 | sp\|Q12824\|SNF5_HUMAN | SMARCB1 | 44.11 | 3.0533 |
| 83 | 98 | sp\|P78527\|PRKDC_HUMAN | PRKDC | 468.79 | 3.3759 |
| 9 | 93 | sp\|P63261\|ACTG_HUMAN | ACTG1 | 41.77 | 3.302 |
| 29 | 89 | sp\|Q92925\|SMRD2_HUMAN | SMARCD2 | 58.88 | 3.204 |
| 13 | 70 | sp\|Q4VC05\|BCL7A_HUMAN | BCL7A | 22.8 | 3.2931 |
| 33 | 69 | sp\|Q9H8M2\|BRD9_HUMAN | BRD9 | 66.96 | 3.3574 |
| 21 | 48 | sp\|P49411\|EFTU_HUMAN | TUFM | 49.51 | 2.863 |
| 17 | 46 | sp\|Q92785\|REQU_HUMAN | DPF2 | 44.13 | 3.6127 |
| 26 | 42 | sp\|P25705\|ATPA_HUMAN | ATP5A1 | 59.71 | 3.5053 |
| 13 | 41 | sp\|P12236\|ADT3_HUMAN | SLC25A6 | 32.85 | 2.6018 |
| 35 | 36 | sp\|P35580\|MYH10_HUMAN | MYH10 | 228.86 | 3.6421 |
| 34 | 34 | sp\|O75643\|U520_HUMAN | SNRNP200 | 244.35 | 3.2978 |
| 28 | 33 | sp\|P06576\|ATPB_HUMAN | ATP5B | 56.52 | 3.6358 |
| 11 | 33 | sp\|Q8WUZ0\|BCL7C_HUMAN | BCL7C | 23.45 | 3.2939 |
| 20 | 29 | sp\|P52272\|HNRPM_HUMAN | HNRNPM | 77.46 | 3.2563 |
| 16 | 29 | sp\|Q6AI39\|BICRL_HUMAN | BICRAL | 115.01 | 3.0716 |
| 25 | 27 | sp\|P35579\|MYH9_HUMAN | MYH9 | 226.39 | 4.1382 |
| 23 | 25 | sp\|Q08211\|DHX9_HUMAN | DHX9 | 140.87 | 3.6402 |
| 20 | 25 | sp\|P05023\|AT1A1_HUMAN | ATP1A1 | 112.82 | 3.7054 |
| 22 | 24 | sp\|Q9UJS0\|CMC2_HUMAN | SLC25A13 | 74.13 | 3.3988 |
| 13 | 24 | sp\|Q00325\|MPCP_HUMAN | SLC25A3 | 40.07 | 2.7509 |
| 18 | 23 | sp\|P68371\|TBB4B_HUMAN | TUBB4B | 49.8 | 3.6902 |
| 17 | 23 | sp\|O95831\|AIFM1_HUMAN | AIFM1 | 66.86 | 3.2073 |
| 21 | 21 | sp\|Q92621\|NU205_HUMAN | NUP205 | 227.78 | 3.3918 |
| 21 | 21 | sp\|Q6P2Q9\|PRP8_HUMAN | PRPF8 | 273.43 | 2.9541 |
| 15 | 20 | sp\|Q9NVI7\|ATD3A_HUMAN | ATAD3A | 71.32 | 3.2389 |
| 9 | 20 | sp\|P68104\|EF1A1_HUMAN | EEF1A1 | 50.11 | 3.3548 |
| 4 | 20 | tr\|B9EGQ8\|B9EGQ8_HUMAN | SMARCA4 | 189.33 | 3.7789 |
| 14 | 19 | sp\|P11021\|GRP78_HUMAN | HSPA5 | 72.29 | 3.312 |
| 18 | 18 | sp\|P52701\|MSH6_HUMAN | MSH6 | 152.69 | 3.2055 |
| 18 | 18 | sp\|Q8N1F7\|NUP93_HUMAN | NUP93 | 93.43 | 2.826 |
| 17 | 17 | sp\|Q10570\|CPSF1_HUMAN | CPSF1 | 160.78 | 3.3065 |
| 12 | 17 | sp\|P20700\|LMNB1_HUMAN | LMNB1 | 66.37 | 3.3674 |
| 14 | 16 | sp\|P38646\|GRP75_HUMAN | HSPA9 | 73.63 | 3.5339 |
| 15 | 15 | sp\|Q15029\|U5S1_HUMAN | EFTUD2 | 109.37 | 3.4816 |
| 15 | 15 | sp\|Q14204\|DYHC1_HUMAN | DYNC1H1 | 532.07 | 3.4659 |
| 15 | 15 | sp\|Q7L0Y3\|MRRP1_HUMAN | TRMT10C | 47.32 | 3.2489 |
| 15 | 15 | sp\|Q9BQG0\|MBB1A_HUMAN | MYBBP1A | 148.76 | 2.8622 |
| 13 | 15 | sp\|Q16891\|MIC60_HUMAN | IMMT | 83.63 | 3.7338 |
| 12 | 15 | sp\|P16615\|AT2A2_HUMAN | ATP2A2 | 114.68 | 2.973 |
| 14 | 14 | sp\|P04844\|RPN2_HUMAN | RPN2 | 69.24 | 3.8313 |
| 14 | 14 | sp\|Q14980\|NUMA1_HUMAN | NUMA1 | 238.12 | 3.6168 |
| 13 | 14 | sp\|Q9BQE3\|TBA1C_HUMAN | TUBA1C | 49.86 | 3.2879 |
| 2 | 14 | sp\|Q15532\|SSXT_HUMAN | SS18 | 45.9 | 2.6617 |
| 13 | 13 | sp\|Q02978\|M2OM_HUMAN | SLC25A11 | 34.04 | 3.6267 |
| 12 | 13 | sp\|P30837\|AL1B1_HUMAN | ALDH1B1 | 57.17 | 3.9563 |
| 12 | 13 | sp\|P42704\|LPPRC_HUMAN | LRPPRC | 157.81 | 3.2168 |
| 11 | 13 | sp\|P22087\|FBRL_HUMAN | FBL | 33.76 | 3.2519 |
| 12 | 12 | sp\|Q96A33\|CCD47_HUMAN | CCDC47 | 55.84 | 3.6465 |
| 12 | 12 | sp\|P10809\|CH60_HUMAN | HSPD1 | 61.02 | 3.3748 |
| 11 | 12 | sp\|P40939\|ECHA_HUMAN | HADHA | 82.95 | 3.7455 |
| 11 | 12 | sp\|P43243\|MATR3_HUMAN | MATR3 | 94.56 | 3.2881 |
| 8 | 12 | sp\|P36542\|ATPG_HUMAN | ATP5C1 | 32.98 | 2.4526 |
| 5 | 12 | sp\|Q9BQE9\|BCL7B_HUMAN | BCL7B | 22.18 | 2.6066 |
| 11 | 11 | sp\|O75746\|CMC1_HUMAN | SLC25A12 | 74.71 | 3.7205 |
| 11 | 11 | sp\|P04181\|OAT_HUMAN | OAT | 48.5 | 3.6399 |
| 11 | 11 | sp\|Q9Y4W6\|AFG32_HUMAN | AFG3L2 | 88.53 | 3.6226 |
| 11 | 11 | sp\|O75306\|NDUS2_HUMAN | NDUFS2 | 52.51 | 3.5563 |
| 11 | 11 | sp\|O43795\|MYO1B_HUMAN | MYO1B | 131.9 | 3.3594 |
| 11 | 11 | sp\|Q9BUQ8\|DDX23_HUMAN | DDX23 | 95.52 | 3.0862 |
| 10 | 11 | sp\|P21796\|VDAC1_HUMAN | VDAC1 | 30.75 | 3.361 |
| 10 | 11 | sp\|Q53H12\|AGK_HUMAN | AGK | 47.11 | 3.1775 |
| 9 | 11 | sp\|Q6UN15\|FIP1_HUMAN | FIP1L1 | 66.49 | 3.5179 |
| 9 | 11 | sp\|Q9H9B4\|SFXN1_HUMAN | SFXN1 | 35.6 | 3.4442 |
| 10 | 10 | sp\|O00567\|NOP56_HUMAN | NOP56 | 66.01 | 3.7799 |
| 10 | 10 | sp\|Q6NUK1\|SCMC1_HUMAN | SLC25A24 | 53.32 | 3.4896 |
| 10 | 10 | sp\|O94832\|MYO1D_HUMAN | MYO1D | 116.13 | 3.1152 |
| 10 | 10 | sp\|Q12769\|NU160_HUMAN | NUP160 | 162.02 | 3.0307 |
| 9 | 10 | sp\|Q53GQ0\|DHB12_HUMAN | HSD17B12 | 34.3 | 3.7669 |
| 9 | 10 | sp\|Q9Y5B6\|PAXB1_HUMAN | PAXBP1 | 104.74 | 3.6482 |
| 9 | 10 | sp\|P11310\|ACADM_HUMAN | ACADM | 46.56 | 3.5587 |
| 9 | 10 | sp\|Q9C0J8\|WDR33_HUMAN | WDR33 | 145.8 | 3.513 |
| 9 | 10 | sp\|Q96I99\|SUCB2_HUMAN | SUCLG2 | 46.48 | 3.1081 |
| 8 | 10 | sp\|P11177\|ODPB_HUMAN | PDHB | 39.21 | 3.3188 |
| 5 | 10 | IGH1M_MOUSE | Ighg1 | 43.36 | 2.8688 |
| 5 | 10 | sp\|P53985\|MOT1_HUMAN | SLC16A1 | 53.91 | 2.6482 |
| 9 | 9 | sp\|Q9H857\|NT5D2_HUMAN | NT5DC2 | 60.68 | 3.0761 |
| 9 | 9 | sp\|Q5SRE5\|NU188_HUMAN | NUP188 | 195.92 | 2.5925 |
| 8 | 9 | sp\|Q92616\|GCN1_HUMAN | GCN1 | 292.57 | 3.5758 |
| 8 | 9 | sp\|Q9UBB9\|TFP11_HUMAN | TFIP11 | 96.76 | 2.5113 |
| 7 | 9 | sp\|Q9P035\|HACD3_HUMAN | HACD3 | 43.13 | 3.1073 |
| 5 | 9 | sp\|Q5T280\|CI114_HUMAN | SPOUT1 | 41.98 | 3.3565 |
| 8 | 8 | sp\|O00159\|MYO1C_HUMAN | MYO1C | 121.61 | 4.128 |
| 8 | 8 | sp\|O75489\|NDUS3_HUMAN | NDUFS3 | 30.22 | 3.6044 |
| 8 | 8 | sp\|Q8NI60\|COQ8A_HUMAN | COQ8A | 71.9 | 3.597 |
| 8 | 8 | sp\|Q969V3\|NCLN_HUMAN | NCLN | 62.93 | 3.2065 |
| 8 | 8 | sp\|P33993\|MCM7_HUMAN | MCM7 | 81.26 | 3.0851 |
| 8 | 8 | sp\|Q9UJV9\|DDX41_HUMAN | DDX41 | 69.79 | 2.6071 |
| 7 | 8 | sp\|P11142\|HSP7C_HUMAN | HSPA8 | 70.85 | 3.9598 |
| 7 | 8 | sp\|O14980\|XPO1_HUMAN | XPO1 | 123.31 | 3.6204 |
| 7 | 8 | sp\|P39656\|OST48_HUMAN | DDOST | 50.77 | 3.2075 |
| 7 | 8 | sp\|O60313\|OPA1_HUMAN | OPA1 | 111.56 | 2.9217 |
| 7 | 8 | sp\|Q8N8A6\|DDX51_HUMAN | DDX51 | 72.41 | 2.8323 |
| 6 | 8 | sp\|P22695\|QCR2_HUMAN | UQCRC2 | 48.41 | 3.6726 |
| 3 | 8 | sp\|P05141\|ADT2_HUMAN | SLC25A5 | 32.83 | 3.2983 |
| 7 | 7 | sp\|Q9Y2X3\|NOP58_HUMAN | NOP58 | 59.54 | 3.9978 |
| 7 | 7 | sp\|P43246\|MSH2_HUMAN | MSH2 | 104.68 | 3.7982 |
| 7 | 7 | sp\|Q9Y2R4\|DDX52_HUMAN | DDX52 | 67.46 | 3.695 |
| 7 | 7 | sp\|Q16822\|PCKGM_HUMAN | PCK2 | 70.68 | 3.5129 |
| 7 | 7 | sp\|A0FGR8\|ESYT2_HUMAN | ESYT2 | 102.29 | 3.375 |
| 7 | 7 | sp\|O14983\|AT2A1_HUMAN | ATP2A1 | 110.18 | 3.3628 |
| 7 | 7 | sp\|Q29RF7\|PDS5A_HUMAN | PDS5A | 150.73 | 3.3405 |
| 7 | 7 | sp\|Q96T37\|RBM15_HUMAN | RBM15 | 107.12 | 3.1355 |
| 7 | 7 | sp\|Q9P2I0\|CPSF2_HUMAN | CPSF2 | 88.43 | 2.6885 |
| 7 | 7 | sp\|P53007\|TXTP_HUMAN | SLC25A1 | 33.99 | 2.3894 |
| 6 | 7 | sp\|O00411\|RPOM_HUMAN | POLRMT | 138.53 | 4.0187 |
| 6 | 7 | sp\|Q3ZCQ8\|TIM50_HUMAN | TIMM50 | 39.62 | 3.4954 |
| 6 | 7 | sp\|P50213\|IDH3A_HUMAN | IDH3A | 39.57 | 2.9987 |
| 6 | 7 | sp\|Q92841\|DDX17_HUMAN | DDX17 | 80.22 | 2.8887 |
| 6 | 7 | sp\|Q14739\|LBR_HUMAN | LBR | 70.66 | 2.7688 |
| 6 | 6 | sp\|Q96TA2\|YMEL1_HUMAN | YME1L1 | 86.4 | 4.3425 |
| 6 | 6 | sp\|Q8TED0\|UTP15_HUMAN | UTP15 | 58.38 | 3.7408 |
| 6 | 6 | sp\|P13674\|P4HA1_HUMAN | P4HA1 | 61.01 | 3.6475 |
| 6 | 6 | sp\|Q3SY69\|AL1L2_HUMAN | ALDH1L2 | 101.68 | 3.5282 |
| 6 | 6 | sp\|P26368\|U2AF2_HUMAN | U2AF2 | 53.47 | 3.4804 |
| 6 | 6 | tr\|F8VXC8\|F8VXC8_HUMAN | SMARCC2 | 136.1 | 3.4048 |
| 6 | 6 | sp\|P45954\|ACDSB_HUMAN | ACADSB | 47.46 | 3.1749 |
| 6 | 6 | sp\|P34931\|HS71L_HUMAN | HSPA1L | 70.33 | 3.111 |
| 6 | 6 | sp\|P26641\|EF1G_HUMAN | EEF1G | 50.09 | 3.109 |
| 6 | 6 | sp\|Q9NUL7\|DDX28_HUMAN | DDX28 | 59.54 | 3.0823 |
| 6 | 6 | sp\|P55084\|ECHB_HUMAN | HADHB | 51.26 | 3.0592 |
| 6 | 6 | sp\|P35251\|RFC1_HUMAN | RFC1 | 128.18 | 3.0236 |
| 6 | 6 | sp\|Q9BPW8\|NIPS1_HUMAN | NIPSNAP1 | 33.29 | 2.9544 |
| 6 | 6 | sp\|Q9NXE4\|NSMA3_HUMAN | SMPD4 | 93.29 | 2.9319 |
| 6 | 6 | sp\|O43615\|TIM44_HUMAN | TIMM44 | 51.32 | 2.8937 |
| 6 | 6 | sp\|P50416\|CPT1A_HUMAN | CPT1A | 88.31 | 2.7685 |
| 5 | 6 | sp\|O43175\|SERA_HUMAN | PHGDH | 56.61 | 3.9221 |
| 5 | 6 | sp\|Q8NDT2\|RB15B_HUMAN | RBM15B | 97.15 | 3.6373 |
| 5 | 6 | sp\|Q15758\|AAAT_HUMAN | SLC1A5 | 56.56 | 3.3323 |
| 5 | 6 | sp\|P53597\|SUCA_HUMAN | SUCLG1 | 36.23 | 3.2971 |
| 5 | 6 | sp\|P50570\|DYN2_HUMAN | DNM2 | 98 | 3.1087 |
| 4 | 6 | sp\|Q92784\|DPF3_HUMAN | DPF3 | 43.06 | 4.3143 |
| 4 | 6 | sp\|P51571\|SSRD_HUMAN | SSR4 | 18.99 | 2.9063 |
| 5 | 5 | sp\|Q68CP9\|ARID2_HUMAN | ARID2 | 197.27 | 3.8774 |
| 5 | 5 | sp\|Q5UIP0\|RIF1_HUMAN | RIF1 | 274.29 | 3.8562 |
| 5 | 5 | sp\|O14828\|SCAM3_HUMAN | SCAMP3 | 38.26 | 3.8314 |
| 5 | 5 | sp\|A1L0T0\|ILVBL_HUMAN | ILVBL | 67.82 | 3.7405 |
| 5 | 5 | sp\|P47985\|UCRI_HUMAN | UQCRFS1 | 29.65 | 3.6437 |
| 5 | 5 | sp\|Q03701\|CEBPZ_HUMAN | CEBPZ | 120.9 | 3.564 |
| 5 | 5 | sp\|P45880\|VDAC2_HUMAN | VDAC2 | 31.55 | 3.5043 |
| 5 | 5 | sp\|Q9BW27\|NUP85_HUMAN | NUP85 | 74.97 | 3.4294 |
| 5 | 5 | sp\|P53621\|COPA_HUMAN | COPA | 138.26 | 3.3088 |
| 5 | 5 | sp\|Q9UDR5\|AASS_HUMAN | AASS | 102.07 | 3.296 |
| 5 | 5 | sp\|P04843\|RPN1_HUMAN | RPN1 | 68.53 | 3.2012 |
| 5 | 5 | sp\|Q9BSD7\|NTPCR_HUMAN | NTPCR | 20.7 | 3.1848 |
| 5 | 5 | sp\|Q6JQN1\|ACD10_HUMAN | ACAD10 | 118.76 | 3.1462 |
| 5 | 5 | sp\|P08670\|VIME_HUMAN | VIM | 53.62 | 3.0791 |
| 5 | 5 | sp\|Q6P4A7\|SFXN4_HUMAN | SFXN4 | 37.97 | 2.9877 |
| 5 | 5 | sp\|Q9NTI5\|PDS5B_HUMAN | PDS5B | 164.56 | 2.9774 |
| 5 | 5 | sp\|P46977\|STT3A_HUMAN | STT3A | 80.48 | 2.9334 |
| 5 | 5 | sp\|Q96EY1\|DNJA3_HUMAN | DNAJA3 | 52.46 | 2.8714 |
| 5 | 5 | sp\|P00367\|DHE3_HUMAN | GLUD1 | 61.36 | 2.6644 |
| 5 | 5 | sp\|Q9NRK6\|ABCBA_HUMAN | ABCB10 | 79.1 | 2.6502 |
| 5 | 5 | sp\|Q9P2R7\|SUCB1_HUMAN | SUCLA2 | 50.29 | 2.0854 |
| 4 | 5 | sp\|Q9H9P8\|L2HDH_HUMAN | L2HGDH | 50.28 | 3.836 |
| 4 | 5 | sp\|Q15637\|SF01_HUMAN | SF1 | 68.29 | 3.8277 |
| 4 | 5 | sp\|P28331\|NDUS1_HUMAN | NDUFS1 | 79.42 | 3.6792 |
| 4 | 5 | sp\|Q9H7H0\|MET17_HUMAN | METTL17 | 50.7 | 3.5298 |
| 4 | 5 | sp\|Q9NPI1\|BRD7_HUMAN | BRD7 | 74.09 | 3.3321 |
| 4 | 5 | sp\|Q14974\|IMB1_HUMAN | KPNB1 | 97.11 | 3.287 |
| 4 | 5 | sp\|P53618\|COPB_HUMAN | COPB1 | 107.07 | 3.1059 |
| 4 | 5 | sp\|P07437\|TBB5_HUMAN | TUBB | 49.64 | 2.9809 |
| 4 | 5 | sp\|Q96CS3\|FAF2_HUMAN | FAF2 | 52.59 | 2.8438 |
| 4 | 4 | sp\|P13804\|ETFA_HUMAN | ETFA | 35.06 | 4.3735 |
| 4 | 4 | sp\|P52597\|HNRPF_HUMAN | HNRNPF | 45.64 | 4.2399 |
| 4 | 4 | sp\|Q96BW9\|TAM41_HUMAN | TAMM41 | 51.03 | 4.2029 |
| 4 | 4 | sp\|Q5T9A4\|ATD3B_HUMAN | ATAD3B | 72.53 | 3.9984 |
| 4 | 4 | sp\|Q9HC07\|TM165_HUMAN | TMEM165 | 34.88 | 3.9971 |
| 4 | 4 | sp\|Q9GZR7\|DDX24_HUMAN | DDX24 | 96.27 | 3.86 |
| 4 | 4 | sp\|Q92947\|GCDH_HUMAN | GCDH | 48.1 | 3.8323 |
| 4 | 4 | sp\|P55795\|HNRH2_HUMAN | HNRNPH2 | 49.23 | 3.6838 |
| 4 | 4 | sp\|Q92576\|PHF3_HUMAN | PHF3 | 229.34 | 3.6333 |
| 4 | 4 | sp\|Q9Y512\|SAM50_HUMAN | SAMM50 | 51.94 | 3.6212 |
| 4 | 4 | sp\|O43837\|IDH3B_HUMAN | IDH3B | 42.16 | 3.5936 |
| 4 | 4 | sp\|P0DMV9\|HS71B_HUMAN | HSPA1B | 70.01 | 3.5795 |
| 4 | 4 | sp\|O15269\|SPTC1_HUMAN | SPTLC1 | 52.71 | 3.5667 |
| 4 | 4 | sp\|P48047\|ATPO_HUMAN | ATP5O | 23.26 | 3.3792 |
| 4 | 4 | sp\|Q96NB2\|SFXN2_HUMAN | SFXN2 | 36.21 | 3.3362 |
| 4 | 4 | sp\|Q96EP5\|DAZP1_HUMAN | DAZAP1 | 43.36 | 3.3271 |
| 4 | 4 | sp\|P49590\|SYHM_HUMAN | HARS2 | 56.85 | 3.2496 |
| 4 | 4 | sp\|Q96D53\|COQ8B_HUMAN | COQ8B | 60.03 | 3.2252 |
| 4 | 4 | sp\|Q14978\|NOLC1_HUMAN | NOLC1 | 73.56 | 3.1412 |
| 4 | 4 | sp\|Q9NSE4\|SYIM_HUMAN | IARS2 | 113.72 | 3.0973 |
| 4 | 4 | sp\|P49756\|RBM25_HUMAN | RBM25 | 100.12 | 3.0237 |
| 4 | 4 | sp\|O75616\|ERAL1_HUMAN | ERAL1 | 48.32 | 2.8867 |
| 4 | 4 | sp\|P17844\|DDX5_HUMAN | DDX5 | 69.1 | 2.8479 |
| 4 | 4 | sp\|P55786\|PSA_HUMAN | NPEPPS | 103.21 | 2.8037 |
| 4 | 4 | sp\|P24539\|AT5F1_HUMAN | ATP5F1 | 28.89 | 2.7676 |
| 4 | 4 | sp\|Q12905\|ILF2_HUMAN | ILF2 | 43.04 | 2.7356 |
| 4 | 4 | sp\|O60762\|DPM1_HUMAN | DPM1 | 29.62 | 2.7256 |
| 4 | 4 | sp\|Q9Y305\|ACOT9_HUMAN | ACOT9 | 49.87 | 2.7236 |
| 4 | 4 | sp\|O60318\|GANP_HUMAN | MCM3AP | 218.27 | 2.6029 |
| 3 | 4 | sp\|P35250\|RFC2_HUMAN | RFC2 | 39.13 | 3.773 |
| 3 | 4 | sp\|O75027\|ABCB7_HUMAN | ABCB7 | 82.59 | 3.4147 |
| 3 | 4 | sp\|Q92782\|DPF1_HUMAN | DPF1 | 42.47 | 2.7282 |
| 3 | 4 | sp\|Q9UM00\|TMCO1_HUMAN | TMCO1 | 21.16 | 2.6108 |
| 3 | 4 | sp\|P46459\|NSF_HUMAN | NSF | 82.54 | 2.133 |
| 3 | 3 | sp\|O75400\|PR40A_HUMAN | PRPF40A | 108.74 | 4.5658 |
| 3 | 3 | sp\|Q14498\|RBM39_HUMAN | RBM39 | 59.34 | 4.3685 |
| 3 | 3 | sp\|O95299\|NDUAA_HUMAN | NDUFA10 | 40.72 | 4.3305 |
| 3 | 3 | sp\|Q9NX63\|MIC19_HUMAN | CHCHD3 | 26.14 | 4.2124 |
| 3 | 3 | sp\|O94906\|PRP6_HUMAN | PRPF6 | 106.86 | 4.1852 |
| 3 | 3 | sp\|A6NJ78\|MET15_HUMAN | METTL15 | 46.09 | 4.1633 |
| 3 | 3 | sp\|Q9H583\|HEAT1_HUMAN | HEATR1 | 242.22 | 4.1273 |
| 3 | 3 | sp\|O00165\|HAX1_HUMAN | HAX1 | 31.6 | 4.0022 |
| 3 | 3 | sp\|Q9H845\|ACAD9_HUMAN | ACAD9 | 68.72 | 3.9765 |
| 3 | 3 | sp\|P48735\|IDHP_HUMAN | IDH2 | 50.88 | 3.946 |
| 3 | 3 | sp\|Q00839\|HNRPU_HUMAN | HNRNPU | 90.53 | 3.9089 |
| 3 | 3 | sp\|Q9BW92\|SYTM_HUMAN | TARS2 | 80.99 | 3.8276 |
| 3 | 3 | sp\|Q96SK2\|TM209_HUMAN | TMEM209 | 62.88 | 3.826 |
| 3 | 3 | sp\|Q9HCM4\|E41L5_HUMAN | EPB41L5 | 81.8 | 3.7752 |
| 3 | 3 | sp\|Q8IY17\|PLPL6_HUMAN | PNPLA6 | 149.9 | 3.7737 |
| 3 | 3 | sp\|P35249\|RFC4_HUMAN | RFC4 | 39.66 | 3.7582 |
| 3 | 3 | sp\|Q9UQ90\|SPG7_HUMAN | SPG7 | 88.18 | 3.7499 |
| 3 | 3 | sp\|Q9Y5M8\|SRPRB_HUMAN | SRPRB | 29.68 | 3.7264 |
| 3 | 3 | sp\|Q14103\|HNRPD_HUMAN | HNRNPD | 38.41 | 3.6717 |
| 3 | 3 | sp\|P38432\|COIL_HUMAN | COIL | 62.57 | 3.6706 |
| 3 | 3 | sp\|Q15120\|PDK3_HUMAN | PDK3 | 46.91 | 3.629 |
| 3 | 3 | sp\|P19105\|ML12A_HUMAN | MYL12A | 19.78 | 3.6243 |
| 3 | 3 | sp\|Q6IAN0\|DRS7B_HUMAN | DHRS7B | 35.1 | 3.6008 |
| 3 | 3 | sp\|P28288\|ABCD3_HUMAN | ABCD3 | 75.43 | 3.5716 |
| 3 | 3 | sp\|P31689\|DNJA1_HUMAN | DNAJA1 | 44.84 | 3.5604 |
| 3 | 3 | sp\|Q49A26\|GLYR1_HUMAN | GLYR1 | 60.52 | 3.5595 |
| 3 | 3 | sp\|Q9H0A0\|NAT10_HUMAN | NAT10 | 115.66 | 3.5176 |
| 3 | 3 | sp\|Q9UKM7\|MA1B1_HUMAN | MAN1B1 | 79.53 | 3.4936 |
| 3 | 3 | sp\|P24468\|COT2_HUMAN | NR2F2 | 45.54 | 3.4642 |
| 3 | 3 | sp\|O75600\|KBL_HUMAN | GCAT | 45.26 | 3.4574 |
| 3 | 3 | sp\|P18074\|ERCC2_HUMAN | ERCC2 | 86.85 | 3.446 |
| 3 | 3 | sp\|O75533\|SF3B1_HUMAN | SF3B1 | 145.74 | 3.3905 |
| 3 | 3 | sp\|Q68CQ7\|GL8D1_HUMAN | GLT8D1 | 41.91 | 3.3887 |
| 3 | 3 | sp\|P50402\|EMD_HUMAN | EMD | 28.98 | 3.3781 |
| 3 | 3 | sp\|O94813\|SLIT2_HUMAN | SLIT2 | 169.76 | 3.3702 |
| 3 | 3 | sp\|Q12931\|TRAP1_HUMAN | TRAP1 | 80.06 | 3.3555 |
| 3 | 3 | sp\|Q8N201\|INT1_HUMAN | INTS1 | 244.14 | 3.3539 |
| 3 | 3 | sp\|Q9NVI1\|FANCI_HUMAN | FANCI | 149.23 | 3.3187 |
| 3 | 3 | sp\|Q9UM54\|MYO6_HUMAN | MYO6 | 149.6 | 3.2785 |
| 3 | 3 | sp\|Q5JTZ9\|SYAM_HUMAN | AARS2 | 107.27 | 3.2518 |
| 3 | 3 | sp\|O15270\|SPTC2_HUMAN | SPTLC2 | 62.88 | 3.2179 |
| 3 | 3 | sp\|P43307\|SSRA_HUMAN | SSR1 | 32.22 | 3.2065 |
| 3 | 3 | sp\|Q5T160\|SYRM_HUMAN | RARS2 | 65.46 | 3.2025 |
| 3 | 3 | sp\|Q9H936\|GHC1_HUMAN | SLC25A22 | 34.45 | 3.1966 |
| 3 | 3 | sp\|P33527\|MRP1_HUMAN | ABCC1 | 171.48 | 3.1945 |
| 3 | 3 | sp\|P31943\|HNRH1_HUMAN | HNRNPH1 | 49.2 | 3.1369 |
| 3 | 3 | sp\|P32189\|GLPK_HUMAN | GK | 61.21 | 3.1141 |
| 3 | 3 | sp\|Q92542\|NICA_HUMAN | NCSTN | 78.36 | 3.0897 |
| 3 | 3 | sp\|O94874\|UFL1_HUMAN | UFL1 | 89.54 | 3.0748 |
| 3 | 3 | sp\|O00400\|ACATN_HUMAN | SLC33A1 | 60.87 | 3.047 |
| 3 | 3 | sp\|Q07021\|C1QBP_HUMAN | C1QBP | 31.34 | 3.0334 |
| 3 | 3 | sp\|O76031\|CLPX_HUMAN | CLPX | 69.18 | 3.0094 |
| 3 | 3 | sp\|Q9NZ01\|TECR_HUMAN | TECR | 36.01 | 2.9946 |
| 3 | 3 | sp\|Q9BWM7\|SFXN3_HUMAN | SFXN3 | 35.48 | 2.993 |
| 3 | 3 | sp\|O43913\|ORC5_HUMAN | ORC5 | 50.25 | 2.9825 |
| 3 | 3 | sp\|Q92945\|FUBP2_HUMAN | KHSRP | 73.07 | 2.977 |
| 3 | 3 | sp\|P12235\|ADT1_HUMAN | SLC25A4 | 33.04 | 2.958 |
| 3 | 3 | sp\|Q9H2S9\|IKZF4_HUMAN | IKZF4 | 64.07 | 2.9251 |
| 3 | 3 | sp\|O43824\|GTPB6_HUMAN | GTPBP6 | 56.85 | 2.9127 |
| 3 | 3 | sp\|O43809\|CPSF5_HUMAN | NUDT21 | 26.21 | 2.8367 |
| 3 | 3 | sp\|Q5SY16\|NOL9_HUMAN | NOL9 | 79.27 | 2.8134 |
| 3 | 3 | sp\|Q9NXF1\|TEX10_HUMAN | TEX10 | 105.61 | 2.7769 |
| 3 | 3 | sp\|Q9BX10\|GTPB2_HUMAN | GTPBP2 | 65.73 | 2.7706 |
| 3 | 3 | sp\|P60660\|MYL6_HUMAN | MYL6 | 16.92 | 2.7542 |
| 3 | 3 | sp\|P09874\|PARP1_HUMAN | PARP1 | 113.01 | 2.7471 |
| 3 | 3 | sp\|Q16795\|NDUA9_HUMAN | NDUFA9 | 42.48 | 2.7254 |
| 3 | 3 | sp\|Q86UT6\|NLRX1_HUMAN | NLRX1 | 107.55 | 2.6517 |
| 3 | 3 | sp\|Q9Y678\|COPG1_HUMAN | COPG1 | 97.66 | 2.637 |
| 3 | 3 | sp\|Q15393\|SF3B3_HUMAN | SF3B3 | 135.49 | 2.5952 |
| 3 | 3 | sp\|Q14684\|RRP1B_HUMAN | RRP1B | 84.38 | 2.5659 |
| 3 | 3 | sp\|P23258\|TBG1_HUMAN | TUBG1 | 51.14 | 2.4401 |
| 3 | 3 | sp\|Q96PK6\|RBM14_HUMAN | RBM14 | 69.45 | 2.1696 |
| 2 | 3 | sp\|P56192\|SYMC_HUMAN | MARS | 101.05 | 3.3287 |
| 2 | 3 | sp\|Q8TCT9\|HM13_HUMAN | HM13 | 41.46 | 3.0227 |
| 2 | 3 | sp\|P40938\|RFC3_HUMAN | RFC3 | 40.53 | 2.7393 |
| 2 | 2 | sp\|Q9NS69\|TOM22_HUMAN | TOMM22 | 15.51 | 4.8805 |
| 2 | 2 | sp\|Q9NVH1\|DJC11_HUMAN | DNAJC11 | 63.24 | 4.8216 |
| 2 | 2 | sp\|Q8IY37\|DHX37_HUMAN | DHX37 | 129.46 | 4.5684 |
| 2 | 2 | sp\|P07910\|HNRPC_HUMAN | HNRNPC | 33.65 | 4.5425 |
| 2 | 2 | sp\|Q8TB37\|NUBPL_HUMAN | NUBPL | 34.06 | 4.5419 |
| 2 | 2 | sp\|P23634\|AT2B4_HUMAN | ATP2B4 | 137.83 | 4.4613 |
| 2 | 2 | sp\|Q99805\|TM9S2_HUMAN | TM9SF2 | 75.73 | 4.4071 |
| 2 | 2 | sp\|O95674\|CDS2_HUMAN | CDS2 | 51.38 | 4.4009 |
| 2 | 2 | sp\|Q6DD88\|ATLA3_HUMAN | ATL3 | 60.5 | 4.3202 |
| 2 | 2 | sp\|Q8N465\|D2HDH_HUMAN | D2HGDH | 56.38 | 4.2164 |
| 2 | 2 | sp\|Q8NF37\|PCAT1_HUMAN | LPCAT1 | 59.11 | 4.138 |
| 2 | 2 | sp\|O95470\|SGPL1_HUMAN | SGPL1 | 63.48 | 4.1063 |
| 2 | 2 | sp\|Q96C36\|P5CR2_HUMAN | PYCR2 | 33.62 | 4.0071 |
| 2 | 2 | sp\|P31040\|SDHA_HUMAN | SDHA | 72.65 | 3.9895 |
| 2 | 2 | sp\|Q5JTV8\|TOIP1_HUMAN | TOR1AIP1 | 66.21 | 3.8342 |
| 2 | 2 | sp\|P52292\|IMA1_HUMAN | KPNA2 | 57.83 | 3.7956 |
| 2 | 2 | sp\|Q5JU69\|TOR2A_HUMAN | TOR2A | 35.69 | 3.7897 |
| 2 | 2 | sp\|P61247\|RS3A_HUMAN | RPS3A | 29.93 | 3.7885 |
| 2 | 2 | sp\|Q9BVA1\|TBB2B_HUMAN | TUBB2B | 49.92 | 3.7692 |
| 2 | 2 | sp\|Q01650\|LAT1_HUMAN | SLC7A5 | 54.97 | 3.7618 |
| 2 | 2 | sp\|Q99623\|PHB2_HUMAN | PHB2 | 33.28 | 3.7184 |
| 2 | 2 | sp\|P61225\|RAP2B_HUMAN | RAP2B | 20.49 | 3.709 |
| 2 | 2 | sp\|Q9UDX5\|MTFP1_HUMAN | MTFP1 | 18 | 3.6947 |
| 2 | 2 | sp\|Q13435\|SF3B2_HUMAN | SF3B2 | 100.16 | 3.6764 |
| 2 | 2 | sp\|O14654\|IRS4_HUMAN | IRS4 | 133.68 | 3.6741 |
| 2 | 2 | sp\|O43143\|DHX15_HUMAN | DHX15 | 90.88 | 3.6482 |
| 2 | 2 | sp\|P51648\|AL3A2_HUMAN | ALDH3A2 | 54.81 | 3.6364 |
| 2 | 2 | sp\|Q8NHH9\|ATLA2_HUMAN | ATL2 | 66.19 | 3.6352 |
| 2 | 2 | sp\|P49821\|NDUV1_HUMAN | NDUFV1 | 50.78 | 3.6288 |
| 2 | 2 | sp\|A3KMH1\|VWA8_HUMAN | VWA8 | 214.69 | 3.5989 |
| 2 | 2 | sp\|P35613\|BASI_HUMAN | BSG | 42.17 | 3.5353 |
| 2 | 2 | sp\|O60264\|SMCA5_HUMAN | SMARCA5 | 121.83 | 3.5339 |
| 2 | 2 | sp\|Q9UBX3\|DIC_HUMAN | SLC25A10 | 31.26 | 3.5314 |
| 2 | 2 | sp\|Q9UBM7\|DHCR7_HUMAN | DHCR7 | 54.45 | 3.5297 |
| 2 | 2 | sp\|Q96GC9\|VMP1_HUMAN | VMP1 | 46.21 | 3.5212 |
| 2 | 2 | sp\|Q9UJZ1\|STML2_HUMAN | STOML2 | 38.51 | 3.4846 |
| 2 | 2 | sp\|P08195\|4F2_HUMAN | SLC3A2 | 67.95 | 3.4622 |
| 2 | 2 | sp\|Q8IXI1\|MIRO2_HUMAN | RHOT2 | 68.07 | 3.4496 |
| 2 | 2 | sp\|Q92544\|TM9S4_HUMAN | TM9SF4 | 74.47 | 3.4436 |
| 2 | 2 | sp\|Q9H061\|T126A_HUMAN | TMEM126A | 21.51 | 3.4371 |
| 2 | 2 | sp\|P62987\|RL40_HUMAN | UBA52 | 14.72 | 3.3766 |
| 2 | 2 | sp\|P56134\|ATPK_HUMAN | ATP5J2 | 10.91 | 3.3667 |
| 2 | 2 | sp\|Q14966\|ZN638_HUMAN | ZNF638 | 220.49 | 3.3667 |
| 2 | 2 | sp\|Q96KK5\|H2A1H_HUMAN | HIST1H2AH | 13.9 | 3.3625 |
| 2 | 2 | sp\|P57088\|TMM33_HUMAN | TMEM33 | 27.96 | 3.3465 |
| 2 | 2 | sp\|P42167\|LAP2B_HUMAN | TMPO | 50.64 | 3.3447 |
| 2 | 2 | sp\|P49755\|TMEDA_HUMAN | TMED10 | 24.96 | 3.3296 |
| 2 | 2 | sp\|O00257\|CBX4_HUMAN | CBX4 | 61.33 | 3.3272 |
| 2 | 2 | sp\|P49792\|RBP2_HUMAN | RANBP2 | 357.97 | 3.3175 |
| 2 | 2 | sp\|P42285\|SK2L2_HUMAN | SKIV2L2 | 117.73 | 3.3157 |
| 2 | 2 | sp\|Q9NRZ9\|HELLS_HUMAN | HELLS | 97.01 | 3.3123 |
| 2 | 2 | sp\|O75251\|NDUS7_HUMAN | NDUFS7 | 23.55 | 3.3089 |
| 2 | 2 | sp\|Q86U86\|PB1_HUMAN | PBRM1 | 192.83 | 3.3065 |
| 2 | 2 | sp\|Q8WYP5\|ELYS_HUMAN | AHCTF1 | 252.34 | 3.2824 |
| 2 | 2 | sp\|Q9Y5J1\|UTP18_HUMAN | UTP18 | 61.96 | 3.2445 |
| 2 | 2 | sp\|Q5TA45\|INT11_HUMAN | INTS11 | 67.62 | 3.2252 |
| 2 | 2 | sp\|Q8IZL8\|PELP1_HUMAN | PELP1 | 119.62 | 3.2022 |
| 2 | 2 | sp\|P19404\|NDUV2_HUMAN | NDUFV2 | 27.37 | 3.2008 |
| 2 | 2 | sp\|Q9H0U3\|MAGT1_HUMAN | MAGT1 | 38.01 | 3.1984 |
| 2 | 2 | sp\|P33778\|H2B1B_HUMAN | HIST1H2BB | 13.94 | 3.1968 |
| 2 | 2 | sp\|Q9P0J0\|NDUAD_HUMAN | NDUFA13 | 16.69 | 3.1856 |
| 2 | 2 | sp\|Q9UNQ2\|DIM1_HUMAN | DIMT1 | 35.21 | 3.1738 |
| 2 | 2 | sp\|Q9BTV4\|TMM43_HUMAN | TMEM43 | 44.85 | 3.1711 |
| 2 | 2 | sp\|Q9NUQ2\|PLCE_HUMAN | AGPAT5 | 42.04 | 3.1429 |
| 2 | 2 | sp\|Q00587\|BORG5_HUMAN | CDC42EP1 | 40.27 | 3.1195 |
| 2 | 2 | sp\|P61619\|S61A1_HUMAN | SEC61A1 | 52.23 | 3.0941 |
| 2 | 2 | sp\|Q9BVP2\|GNL3_HUMAN | GNL3 | 61.95 | 3.0916 |
| 2 | 2 | sp\|Q9ULK4\|MED23_HUMAN | MED23 | 156.37 | 3.0903 |
| 2 | 2 | sp\|Q9NVH0\|EXD2_HUMAN | EXD2 | 70.31 | 3.0877 |
| 2 | 2 | sp\|P51553\|IDH3G_HUMAN | IDH3G | 42.77 | 3.0735 |
| 2 | 2 | sp\|Q8IXI2\|MIRO1_HUMAN | RHOT1 | 70.74 | 3.0472 |
| 2 | 2 | sp\|P54652\|HSP72_HUMAN | HSPA2 | 69.98 | 2.9928 |
| 2 | 2 | sp\|Q969X6\|UTP4_HUMAN | UTP4 | 76.84 | 2.9848 |
| 2 | 2 | sp\|O95573\|ACSL3_HUMAN | ACSL3 | 80.37 | 2.972 |
| 2 | 2 | sp\|P05412\|JUN_HUMAN | JUN | 35.65 | 2.9631 |
| 2 | 2 | sp\|Q9Y2X9\|ZN281_HUMAN | ZNF281 | 96.85 | 2.8979 |
| 2 | 2 | sp\|Q9NNW5\|WDR6_HUMAN | WDR6 | 121.65 | 2.8824 |
| 2 | 2 | sp\|Q13505\|MTX1_HUMAN | MTX1 | 51.44 | 2.8361 |
| 2 | 2 | sp\|Q13123\|RED_HUMAN | IK | 65.56 | 2.8316 |
| 2 | 2 | sp\|Q9NR30\|DDX21_HUMAN | DDX21 | 87.29 | 2.794 |
| 2 | 2 | sp\|Q9H8H2\|DDX31_HUMAN | DDX31 | 94.03 | 2.7931 |
| 2 | 2 | sp\|Q5JVF3\|PCID2_HUMAN | PCID2 | 46 | 2.7734 |
| 2 | 2 | sp\|Q9H300\|PARL_HUMAN | PARL | 42.16 | 2.7484 |
| 2 | 2 | sp\|Q8N684\|CPSF7_HUMAN | CPSF7 | 52.02 | 2.7454 |
| 2 | 2 | sp\|Q9NRG9\|AAAS_HUMAN | AAAS | 59.54 | 2.738 |
| 2 | 2 | sp\|O43347\|MSI1H_HUMAN | MSI1 | 39.1 | 2.7273 |
| 2 | 2 | sp\|O94805\|ACL6B_HUMAN | ACTL6B | 46.85 | 2.7223 |
| 2 | 2 | sp\|P30825\|CTR1_HUMAN | SLC7A1 | 67.59 | 2.6943 |
| 2 | 2 | sp\|Q9P032\|NDUF4_HUMAN | NDUFAF4 | 20.25 | 2.6784 |
| 2 | 2 | sp\|Q10469\|MGAT2_HUMAN | MGAT2 | 51.52 | 2.6709 |
| 2 | 2 | sp\|P38117\|ETFB_HUMAN | ETFB | 27.83 | 2.6625 |
| 2 | 2 | sp\|Q9H2V7\|SPNS1_HUMAN | SPNS1 | 56.59 | 2.6569 |
| 2 | 2 | sp\|Q9HD45\|TM9S3_HUMAN | TM9SF3 | 67.84 | 2.6083 |
| 2 | 2 | sp\|Q96HS1\|PGAM5_HUMAN | PGAM5 | 31.98 | 2.5617 |
| 2 | 2 | sp\|P11166\|GTR1_HUMAN | SLC2A1 | 54.05 | 2.5583 |
| 2 | 2 | sp\|Q9NVP1\|DDX18_HUMAN | DDX18 | 75.36 | 2.4152 |
| 2 | 2 | sp\|P40937\|RFC5_HUMAN | RFC5 | 38.47 | 2.3758 |
| 2 | 2 | sp\|Q92643\|GPI8_HUMAN | PIGK | 45.22 | 2.3695 |
| 2 | 2 | sp\|Q7L3T8\|SYPM_HUMAN | PARS2 | 53.23 | 2.3537 |
| 2 | 2 | sp\|Q96AA3\|RFT1_HUMAN | RFT1 | 60.3 | 2.3164 |
| 2 | 2 | sp\|P12081\|SYHC_HUMAN | HARS | 57.37 | 2.3032 |
| 2 | 2 | sp\|Q8TDD1\|DDX54_HUMAN | DDX54 | 98.53 | 2.3024 |
| 2 | 2 | sp\|Q86TJ2\|TAD2B_HUMAN | TADA2B | 48.44 | 2.2102 |
| 2 | 2 | sp\|Q6PI48\|SYDM_HUMAN | DARS2 | 73.52 | 2.2029 |
| 2 | 2 | sp\|P62851\|RS25_HUMAN | RPS25 | 13.73 | 2.0777 |
| 2 | 2 | sp\|Q9HC21\|TPC_HUMAN | SLC25A19 | 35.49 | 1.7524 |
| 1 | 2 | sp\|A8CG34\|P121C_HUMAN | POM121C | 124.98 | 5.0358 |
| 1 | 2 | sp\|Q15388\|TOM20_HUMAN | TOMM20 | 16.29 | 4.3863 |
| 1 | 2 | sp\|Q01844\|EWS_HUMAN | EWSR1 | 68.44 | 4.2847 |
| 1 | 2 | tr\|C8C3P2\|C8C3P2_HUMAN | DPF1 | 45.07 | 4.2698 |
| 1 | 2 | sp\|Q01130\|SRSF2_HUMAN | SRSF2 | 25.46 | 3.8434 |
| 1 | 2 | sp\|Q9BYX7\|ACTBM_HUMAN | POTEKP | 41.99 | 3.5392 |
| 1 | 2 | sp\|P41252\|SYIC_HUMAN | IARS | 144.41 | 2.8477 |
| 1 | 2 | sp\|Q569K6\|CC157_HUMAN | CCDC157 | 83.89 | 2.5838 |
| 1 | 2 | sp\|Q7Z2K6\|ERMP1_HUMAN | ERMP1 | 100.17 | 2.5131 |
| 1 | 2 | sp\|Q7RTS9\|DYM_HUMAN | DYM | 75.89 | 2.1052 |
| 1 | 1 | sp\|Q92804\|RBP56_HUMAN | TAF15 | 61.79 | 6.3231 |
| 1 | 1 | tr\|B7ZAF6\|B7ZAF6_HUMAN | SUCLA2 | 43.83 | 5.7056 |
| 1 | 1 | sp\|P39210\|MPV17_HUMAN | MPV17 | 19.72 | 5.6581 |
| 1 | 1 | sp\|Q9NXW2\|DJB12_HUMAN | DNAJB12 | 41.79 | 5.557 |
| 1 | 1 | sp\|Q8IXB1\|DJC10_HUMAN | DNAJC10 | 91.02 | 5.3514 |
| 1 | 1 | sp\|Q9Y3D7\|TIM16_HUMAN | PAM16 | 13.82 | 5.3043 |
| 1 | 1 | sp\|O75431\|MTX2_HUMAN | MTX2 | 29.74 | 5.2556 |
| 1 | 1 | tr\|H7BXI1\|H7BXI1_HUMAN | ESYT2 | 97.95 | 5.0949 |
| 1 | 1 | sp\|Q7L2E3\|DHX30_HUMAN | DHX30 | 133.85 | 5.0651 |
| 1 | 1 | sp\|O60306\|AQR_HUMAN | AQR | 171.19 | 5.0401 |
| 1 | 1 | sp\|Q9NU22\|MDN1_HUMAN | MDN1 | 632.42 | 5.0035 |
| 1 | 1 | tr\|S4R341\|S4R341_HUMAN | NOLC1 | 8.05 | 4.9157 |
| 1 | 1 | sp\|Q8NE86\|MCU_HUMAN | MCU | 39.84 | 4.8613 |
| 1 | 1 | sp\|P62314\|SMD1_HUMAN | SNRPD1 | 13.27 | 4.8513 |
| 1 | 1 | sp\|Q7L8L6\|FAKD5_HUMAN | FASTKD5 | 86.52 | 4.8453 |
| 1 | 1 | sp\|Q9UH99\|SUN2_HUMAN | SUN2 | 80.26 | 4.8348 |
| 1 | 1 | tr\|B2R5W2\|B2R5W2_HUMAN | HNRNPC | 31.93 | 4.8315 |
| 1 | 1 | sp\|Q7LGA3\|HS2ST_HUMAN | HS2ST1 | 41.85 | 4.7987 |
| 1 | 1 | sp\|Q9H0H0\|INT2_HUMAN | INTS2 | 134.24 | 4.7963 |
| 1 | 1 | sp\|Q9Y3T9\|NOC2L_HUMAN | NOC2L | 84.87 | 4.7054 |
| 1 | 1 | tr\|F8W7T1\|F8W7T1_HUMAN | DPF3 | 46.42 | 4.6927 |
| 1 | 1 | sp\|Q9Y679\|AUP1_HUMAN | AUP1 | 52.99 | 4.6866 |
| 1 | 1 | sp\|P12004\|PCNA_HUMAN | PCNA | 28.75 | 4.6666 |
| 1 | 1 | sp\|P38435\|VKGC_HUMAN | GGCX | 87.5 | 4.6623 |
| 1 | 1 | sp\|O75529\|TAF5L_HUMAN | TAF5L | 66.11 | 4.6056 |
| 1 | 1 | sp\|Q8WY36\|BBX_HUMAN | BBX | 105.06 | 4.5974 |
| 1 | 1 | sp\|O14925\|TIM23_HUMAN | TIMM23 | 21.93 | 4.5899 |
| 1 | 1 | sp\|Q6NSZ9\|ZSC25_HUMAN | ZSCAN25 | 61.44 | 4.5825 |
| 1 | 1 | sp\|Q13601\|KRR1_HUMAN | KRR1 | 43.64 | 4.5489 |
| 1 | 1 | sp\|Q9BXW9\|FACD2_HUMAN | FANCD2 | 164.02 | 4.517 |
| 1 | 1 | sp\|Q9Y2Q3\|GSTK1_HUMAN | GSTK1 | 25.48 | 4.509 |
| 1 | 1 | sp\|P09622\|DLDH_HUMAN | DLD | 54.14 | 4.4997 |
| 1 | 1 | sp\|P62136\|PP1A_HUMAN | PPP1CA | 37.49 | 4.4917 |
| 1 | 1 | sp\|Q5C9Z4\|NOM1_HUMAN | NOM1 | 96.2 | 4.4584 |
| 1 | 1 | sp\|Q5HYI7\|MTX3_HUMAN | MTX3 | 35.07 | 4.4263 |
| 1 | 1 | sp\|P55265\|DSRAD_HUMAN | ADAR | 135.98 | 4.4253 |
| 1 | 1 | sp\|P17812\|PYRG1_HUMAN | CTPS1 | 66.65 | 4.4181 |
| 1 | 1 | sp\|Q8NBN7\|RDH13_HUMAN | RDH13 | 35.91 | 4.4096 |
| 1 | 1 | sp\|P35232\|PHB_HUMAN | PHB | 29.79 | 4.3597 |
| 1 | 1 | sp\|Q9BT22\|ALG1_HUMAN | ALG1 | 52.48 | 4.3474 |
| 1 | 1 | sp\|Q9BQ67\|GRWD1_HUMAN | GRWD1 | 49.39 | 4.3332 |
| 1 | 1 | sp\|P35558\|PCKGC_HUMAN | PCK1 | 69.15 | 4.2123 |
| 1 | 1 | sp\|Q9UNL2\|SSRG_HUMAN | SSR3 | 21.07 | 4.1982 |
| 1 | 1 | sp\|O60884\|DNJA2_HUMAN | DNAJA2 | 45.72 | 4.1793 |
| 1 | 1 | sp\|Q9Y3A6\|TMED5_HUMAN | TMED5 | 25.99 | 4.1645 |
| 1 | 1 | sp\|P15880\|RS2_HUMAN | RPS2 | 31.3 | 4.1633 |
| 1 | 1 | sp\|Q9NVV4\|PAPD1_HUMAN | MTPAP | 66.13 | 4.1628 |
| 1 | 1 | sp\|Q96QD8\|S38A2_HUMAN | SLC38A2 | 55.99 | 4.1444 |
| 1 | 1 | sp\|Q92797\|SYMPK_HUMAN | SYMPK | 141.06 | 4.142 |
| 1 | 1 | sp\|Q75QN2\|INT8_HUMAN | INTS8 | 113.02 | 4.124 |
| 1 | 1 | sp\|O75528\|TADA3_HUMAN | TADA3 | 48.87 | 4.0685 |
| 1 | 1 | sp\|Q6NTF9\|RHBD2_HUMAN | RHBDD2 | 39.18 | 4.0674 |
| 1 | 1 | sp\|Q5JPH6\|SYEM_HUMAN | EARS2 | 58.65 | 4.0427 |
| 1 | 1 | sp\|Q8N0V3\|RBFA_HUMAN | RBFA | 38.34 | 4.0312 |
| 1 | 1 | sp\|Q9H5Q4\|TFB2M_HUMAN | TFB2M | 45.32 | 4.0306 |
| 1 | 1 | sp\|Q7Z7K6\|CENPV_HUMAN | CENPV | 29.93 | 4.0181 |
| 1 | 1 | sp\|P62995\|TRA2B_HUMAN | TRA2B | 33.65 | 4.0136 |
| 1 | 1 | sp\|P0CG08\|GPHRB_HUMAN | GPR89B | 52.88 | 3.9848 |
| 1 | 1 | sp\|Q9BYD2\|RM09_HUMAN | MRPL9 | 30.22 | 3.9633 |
| 1 | 1 | sp\|Q12788\|TBL3_HUMAN | TBL3 | 88.98 | 3.963 |
| 1 | 1 | sp\|Q8NAN2\|MIGA1_HUMAN | MIGA1 | 70.96 | 3.9529 |
| 1 | 1 | sp\|P57740\|NU107_HUMAN | NUP107 | 106.31 | 3.932 |
| 1 | 1 | sp\|O75439\|MPPB_HUMAN | PMPCB | 54.33 | 3.9211 |
| 1 | 1 | sp\|Q8IWA4\|MFN1_HUMAN | MFN1 | 84.05 | 3.906 |
| 1 | 1 | sp\|Q9UH62\|ARMX3_HUMAN | ARMCX3 | 42.47 | 3.8838 |
| 1 | 1 | sp\|O60830\|TI17B_HUMAN | TIMM17B | 18.26 | 3.8619 |
| 1 | 1 | sp\|Q8TBP6\|S2540_HUMAN | SLC25A40 | 38.1 | 3.8378 |
| 1 | 1 | sp\|Q86Y07\|VRK2_HUMAN | VRK2 | 58.1 | 3.8287 |
| 1 | 1 | sp\|Q96BN2\|TADA1_HUMAN | TADA1 | 37.36 | 3.7447 |
| 1 | 1 | sp\|Q9HDC5\|JPH1_HUMAN | JPH1 | 71.64 | 3.7388 |
| 1 | 1 | sp\|Q6NUN9\|ZN746_HUMAN | ZNF746 | 69.09 | 3.7249 |
| 1 | 1 | sp\|O14579\|COPE_HUMAN | COPE | 34.46 | 3.7075 |
| 1 | 1 | sp\|Q9Y2J2\|E41L3_HUMAN | EPB41L3 | 120.6 | 3.7065 |
| 1 | 1 | sp\|Q8N442\|GUF1_HUMAN | GUF1 | 74.28 | 3.7034 |
| 1 | 1 | sp\|P50454\|SERPH_HUMAN | SERPINH1 | 46.41 | 3.7024 |
| 1 | 1 | sp\|P52429\|DGKE_HUMAN | DGKE | 63.88 | 3.6861 |
| 1 | 1 | sp\|Q96H55\|MYO19_HUMAN | MYO19 | 109.07 | 3.6814 |
| 1 | 1 | sp\|Q9Y2G8\|DJC16_HUMAN | DNAJC16 | 90.53 | 3.6556 |
| 1 | 1 | sp\|Q9Y6J9\|TAF6L_HUMAN | TAF6L | 67.77 | 3.6543 |
| 1 | 1 | sp\|P48651\|PTSS1_HUMAN | PTDSS1 | 55.49 | 3.649 |
| 1 | 1 | sp\|Q08945\|SSRP1_HUMAN | SSRP1 | 81.02 | 3.6213 |
| 1 | 1 | sp\|Q9UBU9\|NXF1_HUMAN | NXF1 | 70.14 | 3.6188 |
| 1 | 1 | sp\|O43159\|RRP8_HUMAN | RRP8 | 50.68 | 3.6157 |
| 1 | 1 | sp\|Q9UMS4\|PRP19_HUMAN | PRPF19 | 55.15 | 3.6146 |
| 1 | 1 | sp\|Q8N6R0\|MET13_HUMAN | METTL13 | 78.72 | 3.5755 |
| 1 | 1 | sp\|Q8TAA9\|VANG1_HUMAN | VANGL1 | 59.94 | 3.5563 |
| 1 | 1 | sp\|O14776\|TCRG1_HUMAN | TCERG1 | 123.82 | 3.556 |
| 1 | 1 | sp\|Q14146\|URB2_HUMAN | URB2 | 170.43 | 3.5345 |
| 1 | 1 | sp\|Q9BVI4\|NOC4L_HUMAN | NOC4L | 58.43 | 3.5218 |
| 1 | 1 | sp\|P35637\|FUS_HUMAN | FUS | 53.39 | 3.4774 |
| 1 | 1 | sp\|P32322\|P5CR1_HUMAN | PYCR1 | 33.34 | 3.4646 |
| 1 | 1 | sp\|Q8N6L1\|KTAP2_HUMAN | KRTCAP2 | 14.67 | 3.4492 |
| 1 | 1 | IGKC_MOUSE | | 11.77 | 3.4442 |
| 1 | 1 | sp\|P37198\|NUP62_HUMAN | NUP62 | 53.22 | 3.4196 |
| 1 | 1 | sp\|Q96RQ1\|ERGI2_HUMAN | ERGIC2 | 42.52 | 3.4166 |
| 1 | 1 | sp\|P49711\|CTCF_HUMAN | CTCF | 82.73 | 3.3935 |
| 1 | 1 | sp\|Q02338\|BDH_HUMAN | BDH1 | 38.13 | 3.3731 |
| 1 | 1 | sp\|Q86VP6\|CAND1_HUMAN | CAND1 | 136.29 | 3.3721 |
| 1 | 1 | sp\|Q86U38\|NOP9_HUMAN | NOP9 | 69.39 | 3.3659 |
| 1 | 1 | sp\|O75352\|MPU1_HUMAN | MPDU1 | 26.62 | 3.3542 |
| 1 | 1 | sp\|Q8WWC4\|MAIP1_HUMAN | MAIP1 | 32.52 | 3.3373 |
| 1 | 1 | sp\|A4D1E9\|GTPBA_HUMAN | GTPBP10 | 42.91 | 3.3326 |
| 1 | 1 | sp\|P17858\|PFKAL_HUMAN | PFKL | 84.96 | 3.3144 |
| 1 | 1 | sp\|Q8WUY9\|DEP1B_HUMAN | DEPDC1B | 61.73 | 3.3118 |
| 1 | 1 | sp\|Q14318\|FKBP8_HUMAN | FKBP8 | 44.53 | 3.275 |
| 1 | 1 | sp\|P17987\|TCPA_HUMAN | TCP1 | 60.31 | 3.2565 |
| 1 | 1 | sp\|P35658\|NU214_HUMAN | NUP214 | 213.49 | 3.2536 |
| 1 | 1 | sp\|Q9NZB8\|MOCS1_HUMAN | MOCS1 | 70.06 | 3.2531 |
| 1 | 1 | sp\|Q96CU9\|FXRD1_HUMAN | FOXRED1 | 53.78 | 3.2527 |
| 1 | 1 | sp\|Q99653\|CHP1_HUMAN | CHP1 | 22.44 | 3.2414 |
| 1 | 1 | sp\|O43306\|ADCY6_HUMAN | ADCY6 | 130.53 | 3.2381 |
| 1 | 1 | sp\|Q8NB90\|SPAT5_HUMAN | SPATA5 | 97.84 | 3.2371 |
| 1 | 1 | sp\|Q6PML9\|ZNT9_HUMAN | SLC30A9 | 63.47 | 3.2286 |
| 1 | 1 | sp\|Q8TCJ2\|STT3B_HUMAN | STT3B | 93.61 | 3.213 |
| 1 | 1 | sp\|Q9UI10\|EI2BD_HUMAN | EIF2B4 | 57.52 | 3.2073 |
| 1 | 1 | sp\|O76021\|RL1D1_HUMAN | RSL1D1 | 54.94 | 3.2001 |
| 1 | 1 | sp\|O75494\|SRS10_HUMAN | SRSF10 | 31.28 | 3.1959 |
| 1 | 1 | sp\|Q8WUK0\|PTPM1_HUMAN | PTPMT1 | 22.83 | 3.1906 |
| 1 | 1 | sp\|P62805\|H4_HUMAN | HIST1H4A | 11.36 | 3.1841 |
| 1 | 1 | sp\|Q15366\|PCBP2_HUMAN | PCBP2 | 38.56 | 3.1618 |
| 1 | 1 | sp\|Q96HW7\|INT4_HUMAN | INTS4 | 108.1 | 3.1575 |
| 1 | 1 | sp\|Q8TE59\|ATS19_HUMAN | ADAMTS19 | 133.96 | 3.1453 |
| 1 | 1 | sp\|Q9NQ50\|RM40_HUMAN | MRPL40 | 24.48 | 3.1291 |
| 1 | 1 | sp\|Q66K74\|MAP1S_HUMAN | MAP1S | 112.14 | 3.1258 |
| 1 | 1 | sp\|Q96JP5\|ZFP91_HUMAN | ZFP91 | 63.41 | 3.1186 |
| 1 | 1 | sp\|P82933\|RT09_HUMAN | MRPS9 | 45.81 | 3.1013 |
| 1 | 1 | sp\|O14981\|BTAF1_HUMAN | BTAF1 | 206.76 | 3.0838 |
| 1 | 1 | sp\|Q14008\|CKAP5_HUMAN | CKAP5 | 225.35 | 3.0668 |
| 1 | 1 | sp\|Q8IZ69\|TRM2A_HUMAN | TRMT2A | 68.68 | 3.0458 |
| 1 | 1 | sp\|Q9H1X3\|DJC25_HUMAN | DNAJC25 | 42.38 | 3.0282 |
| 1 | 1 | sp\|Q7Z406\|MYH14_HUMAN | MYH14 | 227.73 | 3.0134 |
| 1 | 1 | tr\|Q3B7X4\|Q3B7X4_HUMAN | IMMT | 40.47 | 3.0118 |
| 1 | 1 | sp\|Q8WY07\|CTR3_HUMAN | SLC7A3 | 67.13 | 2.9945 |
| 1 | 1 | sp\|Q6UX07\|DHR13_HUMAN | DHRS13 | 40.82 | 2.9688 |
| 1 | 1 | sp\|O00217\|NDUS8_HUMAN | NDUFS8 | 23.69 | 2.948 |
| 1 | 1 | sp\|Q9Y3D6\|FIS1_HUMAN | FIS1 | 16.93 | 2.9299 |
| 1 | 1 | sp\|Q92522\|H1X_HUMAN | H1FX | 22.47 | 2.9236 |
| 1 | 1 | sp\|Q9H490\|PIGU_HUMAN | PIGU | 50.02 | 2.9229 |
| 1 | 1 | sp\|P49368\|TCPG_HUMAN | CCT3 | 60.5 | 2.917 |
| 1 | 1 | sp\|Q9H974\|QTRT2_HUMAN | QTRT2 | 46.68 | 2.9127 |
| 1 | 1 | sp\|Q8WUB8\|PHF10_HUMAN | PHF10 | 56.02 | 2.9119 |
| 1 | 1 | KV2A7_MOUSE | | 12.27 | 2.8807 |
| 1 | 1 | sp\|Q9NXS2\|QPCTL_HUMAN | QPCTL | 42.9 | 2.8763 |
| 1 | 1 | sp\|Q9Y584\|TIM22_HUMAN | TIMM22 | 20.02 | 2.8643 |
| 1 | 1 | sp\|P42356\|PI4KA_HUMAN | PI4KA | 236.68 | 2.8615 |
| 1 | 1 | sp\|Q86TW2\|ADCK1_HUMAN | ADCK1 | 60.54 | 2.8425 |
| 1 | 1 | sp\|Q8TAE8\|G45IP_HUMAN | GADD45GIP1 | 25.37 | 2.8196 |
| 1 | 1 | sp\|O15523\|DDX3Y_HUMAN | DDX3Y | 73.11 | 2.8124 |
| 1 | 1 | sp\|O75323\|NIPS2_HUMAN | NIPSNAP2 | 33.72 | 2.7929 |
| 1 | 1 | sp\|Q9UBD5\|ORC3_HUMAN | ORC3 | 82.2 | 2.7077 |
| 1 | 1 | sp\|O95070\|YIF1A_HUMAN | YIF1A | 31.99 | 2.6979 |
| 1 | 1 | sp\|P42695\|CNDD3_HUMAN | NCAPD3 | 168.78 | 2.6917 |
| 1 | 1 | sp\|P06493\|CDK1_HUMAN | CDK1 | 34.07 | 2.6561 |
| 1 | 1 | sp\|Q09161\|NCBP1_HUMAN | NCBP1 | 91.78 | 2.6544 |
| 1 | 1 | sp\|Q8IUX1\|T126B_HUMAN | TMEM126B | 25.93 | 2.6456 |
| 1 | 1 | tr\|B3KUE6\|B3KUE6_HUMAN | | 30.19 | 2.6448 |
| 1 | 1 | sp\|Q9BVK8\|TM147_HUMAN | TMEM147 | 25.24 | 2.642 |
| 1 | 1 | sp\|Q53R41\|FAKD1_HUMAN | FASTKD1 | 97.35 | 2.6172 |
| 1 | 1 | sp\|O00483\|NDUA4_HUMAN | NDUFA4 | 9.36 | 2.6166 |
| 1 | 1 | sp\|P61026\|RAB10_HUMAN | RAB10 | 22.53 | 2.596 |
| 1 | 1 | sp\|Q96A65\|EXOC4_HUMAN | EXOC4 | 110.43 | 2.5958 |
| 1 | 1 | tr\|Q6FI97\|Q6FI97_HUMAN | BAF53A | 47.35 | 2.5795 |
| 1 | 1 | sp\|Q15392\|DHC24_HUMAN | DHCR24 | 60.06 | 2.5722 |
| 1 | 1 | sp\|P83731\|RL24_HUMAN | RPL24 | 17.77 | 2.5706 |
| 1 | 1 | sp\|Q9NZJ7\|MTCH1_HUMAN | MTCH1 | 41.52 | 2.5516 |
| 1 | 1 | sp\|Q9BUN8\|DERL1_HUMAN | DERL1 | 28.78 | 2.5224 |
| 1 | 1 | sp\|P46783\|RS10_HUMAN | RPS10 | 18.89 | 2.5189 |
| 1 | 1 | sp\|O43390\|HNRPR_HUMAN | HNRNPR | 70.9 | 2.5031 |
| 1 | 1 | sp\|O60673\|REV3L_HUMAN | REV3L | 352.55 | 2.4985 |
| 1 | 1 | sp\|Q8IVH4\|MMAA_HUMAN | MMAA | 46.51 | 2.4963 |
| 1 | 1 | sp\|Q16563\|SYPL1_HUMAN | SYPL1 | 28.55 | 2.4909 |
| 1 | 1 | sp\|O14735\|CDIPT_HUMAN | CDIPT | 23.52 | 2.475 |
| 1 | 1 | sp\|Q9BSJ2\|GCP2_HUMAN | TUBGCP2 | 102.47 | 2.4439 |
| 1 | 1 | sp\|P62701\|RS4X_HUMAN | RPS4X | 29.58 | 2.4418 |
| 1 | 1 | sp\|Q14683\|SMC1A_HUMAN | SMC1A | 143.14 | 2.4391 |
| 1 | 1 | sp\|P08559\|ODPA_HUMAN | PDHA1 | 43.27 | 2.4288 |
| 1 | 1 | sp\|Q9Y651\|SOX21_HUMAN | SOX21 | 28.56 | 2.4124 |
| 1 | 1 | sp\|Q96JX3\|SRAC1_HUMAN | SERAC1 | 74.1 | 2.3968 |
| 1 | 1 | sp\|Q8NDZ4\|DIA1_HUMAN | C3orf58 | 49.45 | 2.3757 |
| 1 | 1 | sp\|P46013\|KI67_HUMAN | MKI67 | 358.47 | 2.3711 |
| 1 | 1 | sp\|P68871\|HBB_HUMAN | HBB | 15.99 | 2.3665 |
| 1 | 1 | sp\|P12036\|NFH_HUMAN | NEFH | 112.41 | 2.3587 |
| 1 | 1 | sp\|Q5T0B9\|ZN362_HUMAN | ZNF362 | 45.79 | 2.3304 |
| 1 | 1 | sp\|Q9H0D6\|XRN2_HUMAN | XRN2 | 108.51 | 2.3224 |
| 1 | 1 | tr\|Q96DP0\|Q96DP0_HUMAN | | 49.22 | 2.2814 |
| 1 | 1 | sp\|O60725\|ICMT_HUMAN | ICMT | 31.92 | 2.2483 |
| 1 | 1 | sp\|Q8N4U5\|T11L2_HUMAN | TCP11L2 | 58.05 | 2.2363 |
| 1 | 1 | sp\|Q9UL03\|INT6_HUMAN | INTS6 | 100.33 | 2.1871 |
| 1 | 1 | sp\|P43007\|SATT_HUMAN | SLC1A4 | 55.69 | 2.1855 |
| 1 | 1 | sp\|Q6VAB6\|KSR2_HUMAN | KSR2 | 107.56 | 2.1832 |
| 1 | 1 | sp\|Q9NWU5\|RM22_HUMAN | MRPL22 | 23.63 | 2.1812 |
| 1 | 1 | tr\|B4DL14\|B4DL14_HUMAN | | 27.5 | 2.1811 |
| 1 | 1 | sp\|Q6YN16\|HSDL2_HUMAN | HSDL2 | 45.37 | 2.1314 |
| 1 | 1 | sp\|Q9NVH2\|INT7_HUMAN | INTS7 | 106.77 | 2.1225 |
| 1 | 1 | sp\|P41219\|PERI_HUMAN | PRPH | 53.62 | 2.117 |
| 1 | 1 | sp\|Q9P0U1\|TOM7_HUMAN | TOMM7 | 6.24 | 2.094 |
| 1 | 1 | sp\|P01857\|IGHG1_HUMAN | IGHG1 | 36.08 | 2.092 |
| 1 | 1 | sp\|Q9Y4F1\|FARP1_HUMAN | FARP1 | 118.56 | 2.0479 |
| 1 | 1 | sp\|O75964\|ATP5L_HUMAN | ATP5L | 11.42 | 2.0435 |
| 1 | 1 | tr\|A0A1W2PP06\|A0A1W2PP06_HUMAN | KCNMA1 | 136.96 | 2.0205 |
| 1 | 1 | sp\|Q92604\|LGAT1_HUMAN | LPGAT1 | 43.06 | 2.0192 |
| 1 | 1 | sp\|Q8WZA0\|LZIC_HUMAN | LZIC | 21.48 | 1.9913 |

TABLE 5E
Ubiquitination report
HA-SS18SSX_CHR

| | | ↓ | | | | | | Gene |
|---|---|---|---|---|---|---|---|---|
| ScanF | Z | XCorr | ΔCorr | # Ions | Reference | Redun | Peptide | Symbol |
| 18257 | 3 | 4.935 | 0.033 | 30/76 | sp\|Q96KK5\|H2A1H_HUMAN | 19 | K.VTIAQGGVLPNIQAVLLPK#K.T | HIST1H2AH |
| | | | | | | | (SEQ ID NO: 229) | |

Purification of SS18-SSX-bound complexes followed by density sedimentation using 10-30% glycerol gradients revealed larger-sized fusion-containing BAF complexes migrating in fractions 15-19, compared to WT SS18-bound complexes in fractions 13-14, as expected (Mashtalir et al. (2018) Cell 175:1272-1288), indicating high-affinity, stable binding of SS18-SSX-bound BAF complexes to the full histone octamer (FIG. 1C and FIG. 2H). In addition, histones bound to the ATPase module components were observed in isolation as well as to free SS18-SSX in fractions 9-13 and 2-4, respectively. These results indicate that fusion-containing BAF complexes exhibit exceptionally strong binding to nucleosomes, able to withstand separation even in a high centrifugal force environment, in contrast to WT BAF complexes which exhibit weaker interactions with nucleosomes, as seen consistently in BAF complex purifications performed to date (Mashtalir et al. (2018) Cell 175:1272-1288). Finally, to determine the relative chromatin affinities of WT BAF complexes versus SS18-SSX-containing BAF complexes, differential salt extraction in both SS cell lines and HEK-293T cells expressing SS18-SSX was performed (FIG. 1D, FIGS. 2C and 2D). Normal extraction profiles for WT complexes were observed (elution at 300-500 mM NaCl), consistent with previous findings (Nakayama et al. (2017) Nat. Genet. 49:1613-1623; Pan et al. (2019) Nat. Genet. 51:618-626). However, fusion-containing complexes remained insoluble in up to 1M NaCl. In support of these findings, fluorescence recovery after photobleaching (FRAP) experiments in HEK-293T cells infected with either GFP-SS18 or GFP-SS18-SSX revealed substantially increased chromatin residency times for SS18-SSX-bound BAF complexes (FIG. 1E and FIG. 2E). Taken together, these findings indicate an unexpected, uniquely high-affinity conjugation of SS18-SSX-bound BAF complexes to nucleosomes, a property specific to this disease-associated BAF complex perturbation, indicating this as a feature that can underlie the site-specific targeting of SS18-SSX complexes on chromatin.

### Example 3: A Minimal 34-Aa Region of SSX is Necessary and Sufficient for Direct Binding to Repressive Nucleosomes and SS18-SSX-Mediated Oncogenic Functions

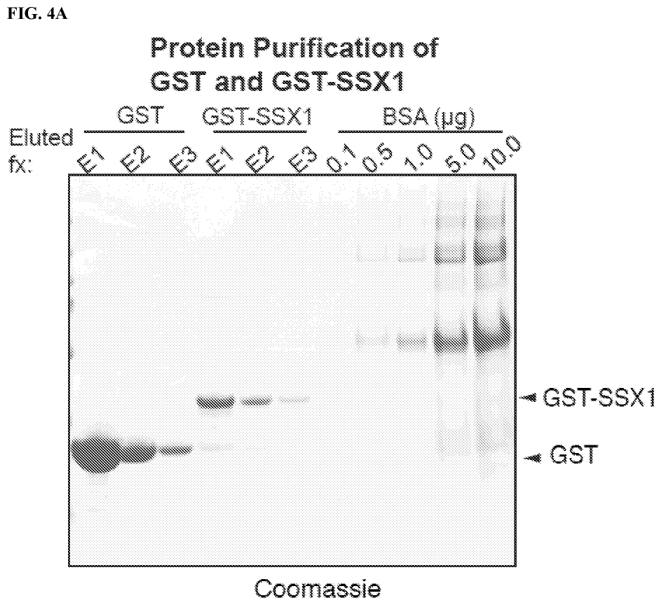

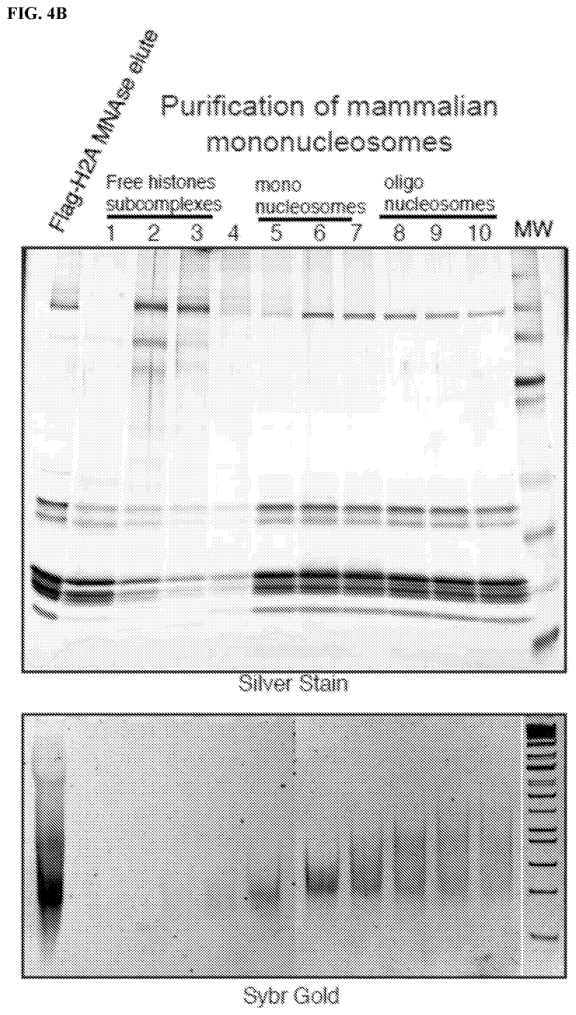

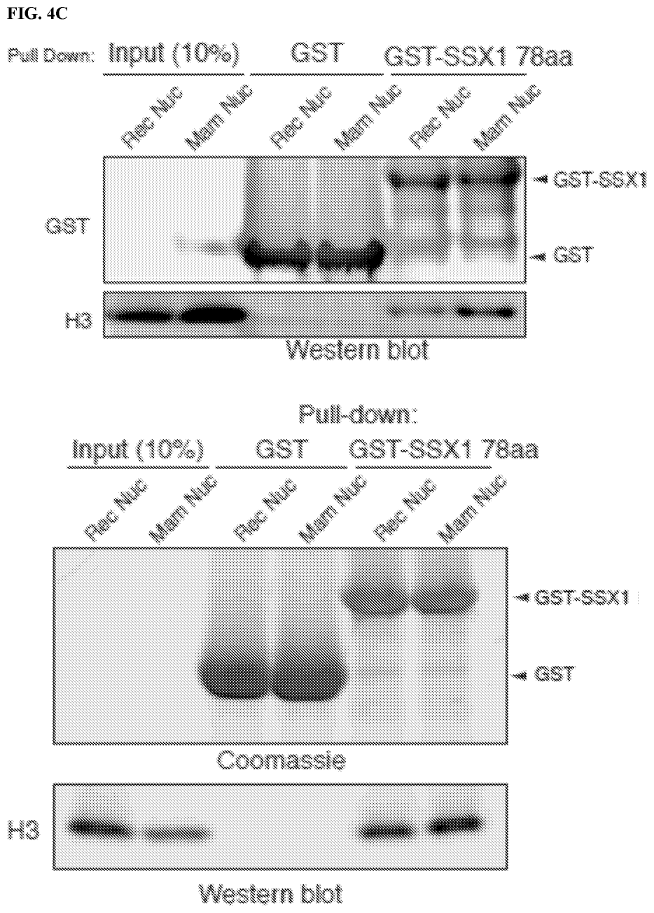

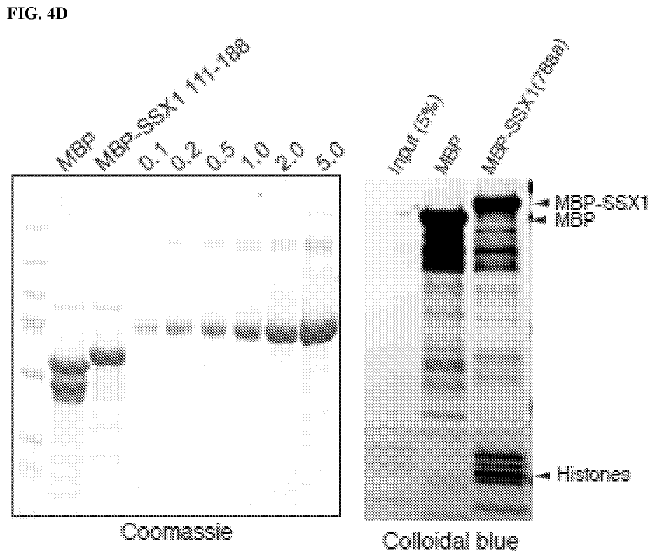

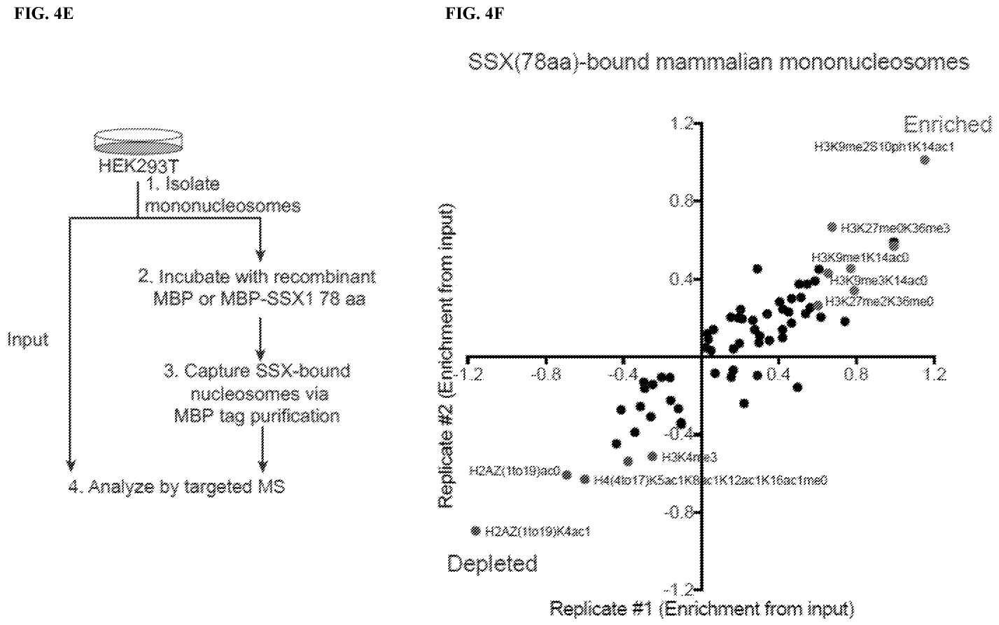

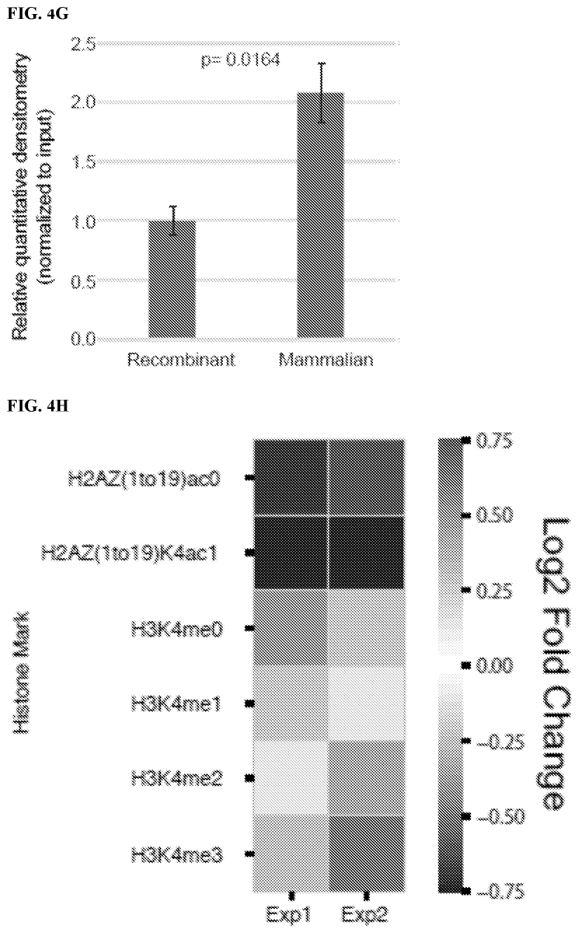

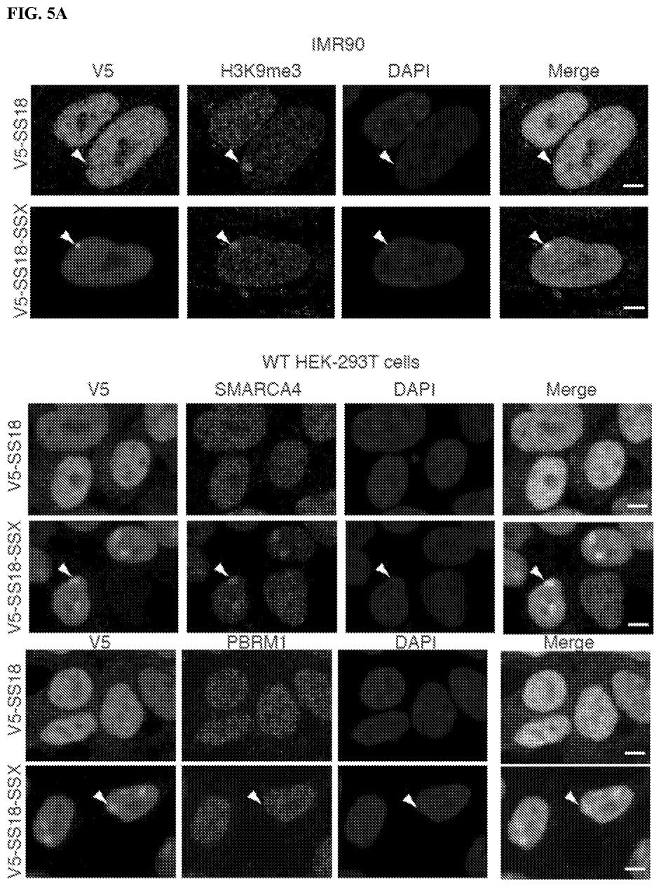

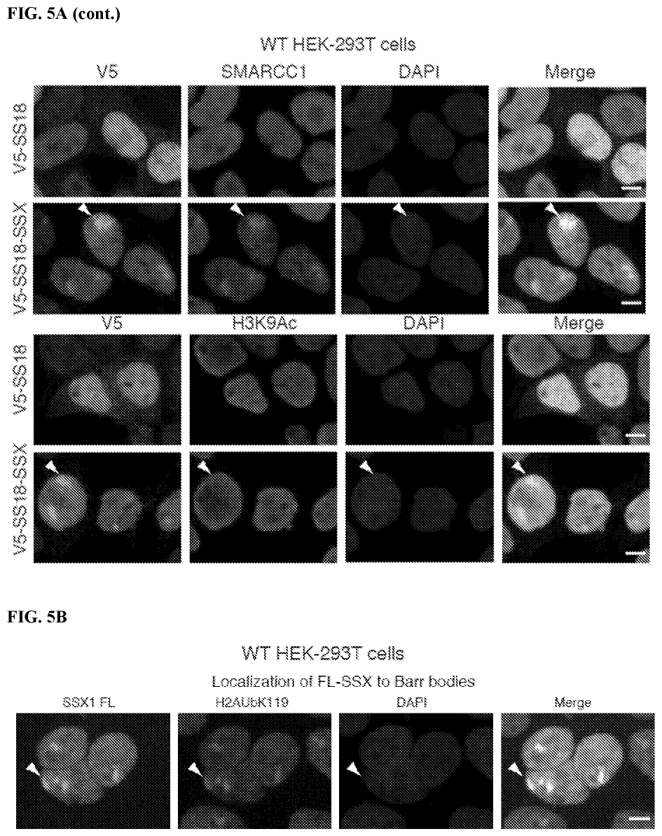

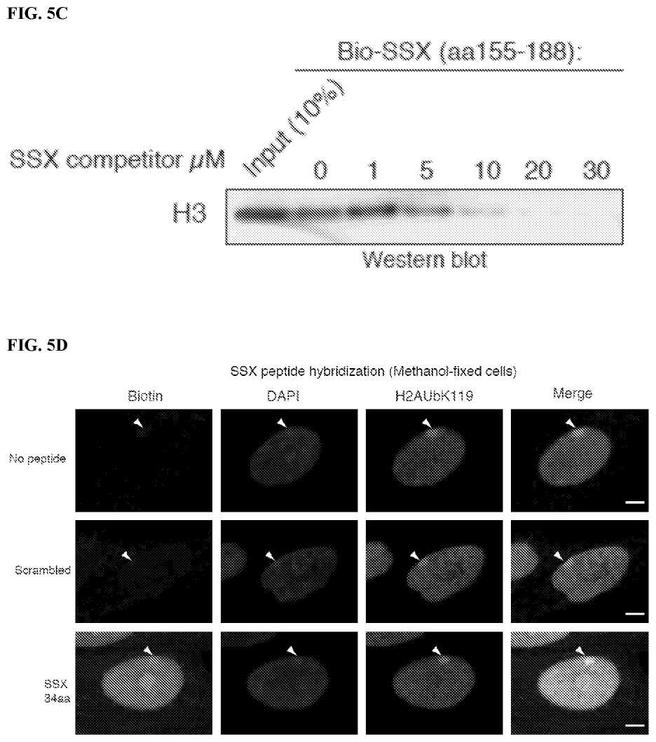

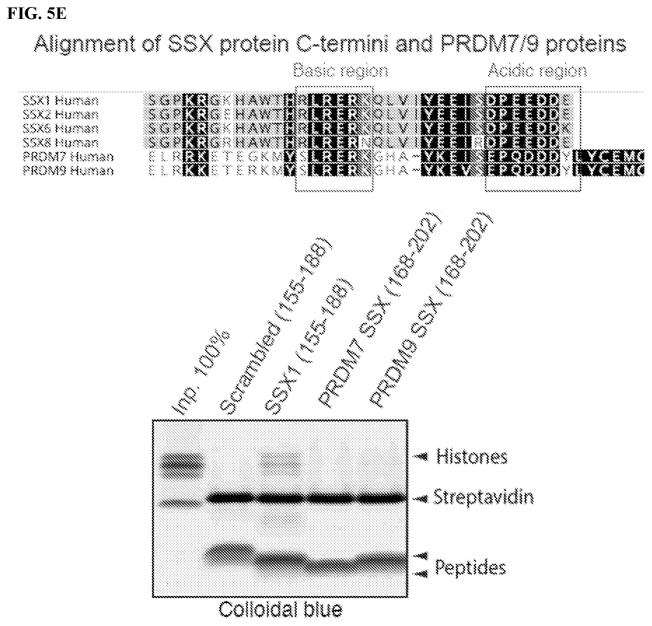

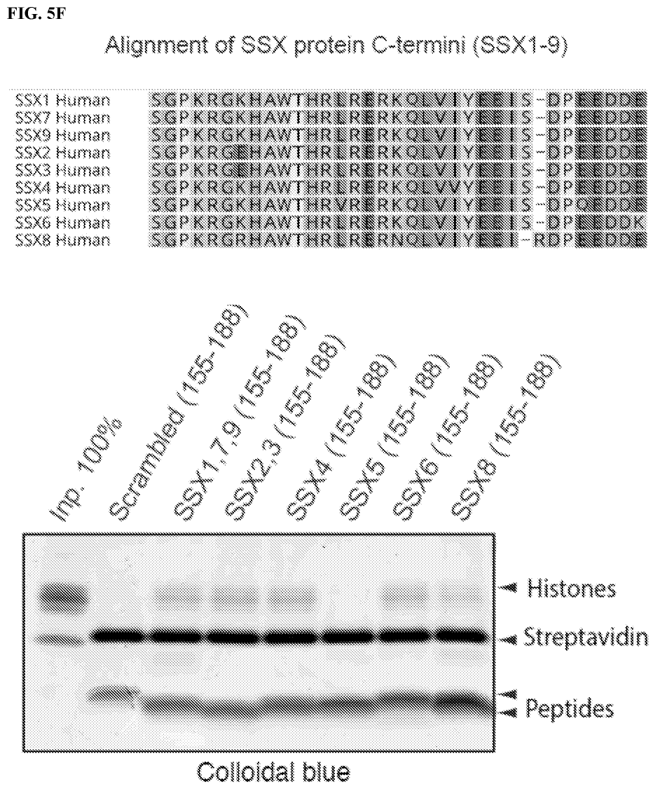

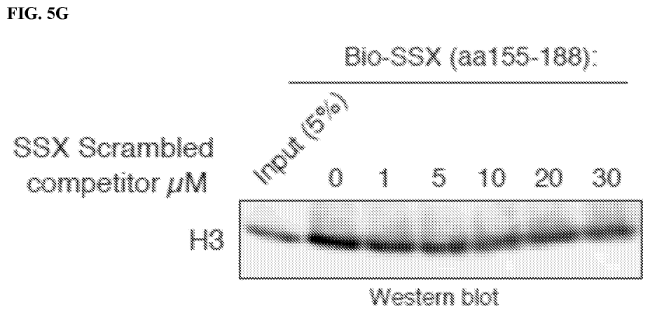

Given these results, it was next determined whether the 78 residues of SSX in isolation (not fused to the SS18 subunit and hence not part of BAF complexes) could directly bind nucleosomes and could be responsible for conferring the unique affinity and nucleosome binding properties of the SS18-SSX fusion protein. Indeed, pull-down experiments revealed that the C-terminal 78 residues of SSX (aa 111-188) were sufficient for its nucleosomal interactions (FIG. 3A and FIGS. 4A-4B). In addition, it was found that binding to mammalian nucleosomes (purified via MNase digestion of HEK-293T cell chromatin and hence representing the diverse array of histone variants and modifications) was stronger than binding to recombinant, unmodified nucleosomes (FIGS. 4C and 4G), indicating that a mammalian histone modification can provide added affinity and site specificity. In agreement with this, targeted quantitative mass-spectrometry (MS) analysis of SSX-bound mammalian nucleosomes (pooled, purified by MNase digestion from HEK-293T cells, containing the full diversity of histone marks) revealed strong enrichment of nucleosomes decorated with known repressive histone marks and depletion of nucleosomes marked with known activation marks (FIG. 3B, FIGS. 4D-4F, and FIG. 4H, Tables 6A-6C). For example, SSX-mediated enrichment of nucleosomes decorated with repressive marks such as H3K27me3 and H3K9me3, and SSX-mediated depletion of nucleosomes decorated with activating marks such as H4 lysine acetylation and H3K4me2/3 were detected (while nucleosomes containing unmodified H4 and H3 were enriched). Further, immunofluorescence (IF) analyses revealed strong colocalization of SS18-SSX as well as SSX in isolation (SSX aa 1-188, as expressed in testes) to Barr bodies marked with repressive PRC1 and PRC2 complexes and their marks (FIG. 3C and FIGS. 5A-5B).

TABLE 6A
log2norm: (Light/Heavy) intensity ratios are normalized to the (Light/Heavy) intensity ratio of
respective histone “norm” peptide, and brought into log2 space. Cells with #N/A were below l.o.d

| | Histone Mark | | | | | |
|---|---|---|---|---|---|---|
| | | MM01_A01 | MM02_B01 | MM03_C01 | MM04_D01 | MM05_E01 |
| | | Cell Line | | | | |
| | | HEK293T | HEK293T | HEK293T | HEK293T | HEK293T |
| | | Perturbation | | | | |
| | | pull down | pull down | pull down | SSX1 | SSX1 |
| | Parent Peptide | input | input | input | pulldown | pulldo |
| Experiment | | 1 | 1 | 1 | 1 | 1 |
| H4(4to17)ac0me0 | H4K5K8K12K16 | −0.999 | −0.768 | −0.681 | −0.613 | −0.573 |
| H4(4to17)K5ac1me0 | H4K5K8K12K16 | 0.048 | 0.332 | 0.333 | −0.053 | 0.075 |
| H4(4to17)K12ac1me0 | H4K5K8K12K16 | −0.327 | 0.036 | 0.140 | −0.207 | −0.275 |
| H4(4to17)K16ac1me0 | H4K5K8K12K16 | −0.265 | −0.168 | −0.258 | −0.391 | −0.337 |
| H4(4to17)K8ac1K12ac1me0 | H4K5K8K12K16 | −0.117 | 0.026 | 0.240 | −0.198 | −0.093 |
| H4(4to17)K5ac1K8ac1me0 | H4K5K8K12K16 | 0.502 | 0.475 | 0.651 | 0.107 | 0.094 |
| H4(4to17)K5ac1K16ac1me0 | H4K5K8K12K16 | 0.618 | 0.676 | 0.621 | 0.226 | 0.367 |
| H4(4to17)K12ac1K16ac1me0 | H4K5K8K12K16 | 0.411 | 0.391 | 0.424 | 0.204 | 0.304 |
| H4(4to17)K5ac1K8ac1K12ac1me0 | H4K5K8K12K16 | −0.054 | 0.091 | 0.312 | −0.223 | −0.270 |
| H4(4to17)K8ac1K12ac1K16ac1me0 | H4K5K8K12K16 | 0.816 | 0.897 | 1.090 | 0.677 | 0.625 |
| H4(4to17)K5ac1K8ac1K16ac1me0 | H4K5K8K12K16 | 0.819 | 0.755 | 0.972 | 0.474 | 0.311 |
| H4(4to17)K5ac1K8ac1K12ac1K16ac1me0 | H4K5K8K12K16 | 0.718 | 0.864 | 1.040 | 0.276 | 0.245 |
| H4(20to23)K20me0 | H4K20 | −0.045 | −0.175 | 0.077 | 0.017 | 0.092 |
| H4(20to23)K20me1 | H4K20 | −0.658 | −0.526 | −0.731 | −0.427 | −0.443 |
| H4(20to23)K20me2 | H4K20 | −0.424 | −0.453 | −0.437 | −0.414 | −0.391 |
| H4(20to23)K20me3 | H4K20 | 0.161 | 0.261 | 0.136 | −0.109 | 0.055 |
| H2AZ(1to19)ac0 | H2A.Z | −0.160 | −0.062 | −0.047 | −0.784 | −0.697 |
| H2AZ(1to19)K4ac1 | H2A.Z | 0.890 | 1.031 | 0.906 | −0.217 | 0.048 |
| H2B(1to29)ac0 | H2B | 0.112 | −0.187 | −0.042 | 0.152 | 0.160 |
| H2B(1to29)K5ac1 | H2B | 0.234 | 0.001 | 0.124 | 0.273 | 0.323 |
| H2A(4to11)ac0 | H2AK5K9 | −0.187 | −0.105 | 0.115 | −0.026 | 0.061 |
| H2A(4to11)K5ac1 | H2AK5K9 | 0.275 | 0.713 | 0.614 | 0.569 | 0.624 |
| H2A(4to11)K9ac1 | H2AK5K9 | −0.837 | −0.552 | −0.586 | −0.254 | −0.375 |
| H2A(4to11)K5ac1K9ac1 | H2AK5K9 | −0.224 | −0.043 | 0.025 | −0.031 | −0.049 |
| H2A(12to17)ac0 | H2AK13K15 | −0.521 | −0.203 | 0.070 | 0.201 | −0.120 |
| H2A(12to17)K13ac1 | H2AK13K15 | −0.459 | −0.129 | −0.116 | 0.032 | −0.047 |
| H2A(12to17)K15ac1 | H2AK13K15 | −0.430 | −0.070 | 0.035 | 0.042 | −0.086 |
| H3K4me0 | H3K4 | −0.049 | 0.108 | 0.047 | 0.549 | 0.339 |
| H3K4me1 | H3K4 | 0.393 | 0.542 | 0.415 | 0.740 | 0.353 |
| H3K4me2 | H3K4 | 0.848 | 0.992 | 0.801 | 0.778 | 0.532 |
| H3K4me3 | H3K4 | 0.953 | 1.142 | 0.953 | 0.766 | 0.505 |
| H3K4ac1 | H3K4 | 0.485 | 0.651 | 0.588 | 0.263 | 0.320 |
| H3K9me0K14ac0 | H3K9K14 | −0.262 | −0.323 | −0.280 | 0.274 | −0.036 |
| H3K9me1K14ac0 | H3K9K14 | −0.501 | −0.392 | −0.314 | 0.206 | 0.046 |
| H3K9me2K14ac0 | H3K9K14 | −0.585 | −0.359 | −0.568 | 0.035 | −0.284 |
| H3K9me3K14ac0 | H3K9K14 | −0.364 | −0.401 | −0.153 | 0.348 | 0.123 |
| H3K9ac1K14ac0 | H3K9K14 | 1.020 | 1.120 | 0.965 | 1.203 | 1.075 |
| H3K9me0K14ac1 | H3K9K14 | 0.271 | 0.259 | −0.018 | 0.637 | 0.469 |
| H3K9me1K14ac1 | H3K9K14 | 0.565 | 0.691 | 0.585 | 0.769 | 0.508 |
| H3K9me2K14ac1 | H3K9K14 | 0.390 | 0.458 | 0.421 | 0.724 | 0.530 |
| H3K9me3K14ac1 | H3K9K14 | 0.657 | 0.663 | 0.268 | 0.828 | 0.602 |
| H3K9ac1K14ac1 | H3K9K14 | 1.842 | 1.990 | 1.786 | 2.038 | 1.804 |
| H3K9me0S10ph1K14ac0 | H3K9K14 | −2.614 | −3.006 | −2.823 | #N/A | #N/A |
| H3K9me1S10ph1K14ac0 | H3K9K14 | −2.261 | −2.090 | −2.268 | #N/A | #N/A |
| H3K9me2S10ph1K14ac0 | H3K9K14 | −2.004 | −1.899 | −2.048 | −0.683 | −1.092 |
| H3K9me3S10ph1K14ac0 | H3K9K14 | −0.920 | −0.606 | −0.834 | −0.566 | −1.026 |
| H3K9me0S10ph1K14ac1 | H3K9K14 | −2.471 | −2.280 | −1.882 | #N/A | #N/A |
| H3K9me1S10ph1K14ac1 | H3K9K14 | −2.299 | −3.153 | −3.422 | −2.668 | −2.507 |
| H3K9me2S10ph1K14ac1 | H3K9K14 | −1.957 | −2.023 | −2.085 | −0.870 | −1.009 |
| H3K18ac0K23ac0 | H3K18K23 | −0.505 | −0.140 | −0.317 | 0.268 | 0.069 |
| H3K18ac1K23ac0 | H3K18K23 | 1.168 | 1.411 | 1.229 | 1.341 | 1.185 |
| H3K18ac0K23ac1 | H3K18K23 | 0.768 | 1.061 | 0.574 | 1.141 | 1.021 |
| H3K18ac1K23ac1 | H3K18K23 | 2.134 | 2.522 | 2.107 | 2.137 | 1.988 |
| H3K27me0K36me0 | H3K27K36 | −0.169 | 0.187 | −0.678 | 0.552 | 0.233 |
| H3K27me0K36me1 | H3K27K36 | −0.407 | −0.152 | −0.659 | 0.384 | −0.066 |
| H3K27me0K36me2 | H3K27K36 | −0.476 | −0.061 | −0.419 | 0.674 | 0.270 |
| H3K27me0K36me3 | H3K27K36 | −0.057 | −0.001 | 0.070 | 0.678 | 0.669 |
| H3K27me1K36me0 | H3K27K36 | −0.086 | 0.026 | −0.148 | 0.396 | 0.104 |
| H3K27me1K36me1 | H3K27K36 | −0.194 | 0.078 | −0.086 | 0.535 | 0.196 |
| H3K27me1K36me2 | H3K27K36 | 0.248 | 0.397 | 0.323 | 0.741 | 0.461 |
| H3K27me1K36me3 | H3K27K36 | 0.229 | 0.344 | 0.296 | 1.283 | 0.859 |
| H3K27me2K36me0 | H3K27K36 | −0.337 | −0.004 | −0.152 | 0.382 | 0.209 |
| H3K27me2K36me1 | H3K27K36 | −0.092 | −0.102 | 0.017 | 0.363 | 0.186 |
| H3K27me2K36me2 | H3K27K36 | 0.249 | 0.342 | 0.242 | 0.553 | 0.417 |
| H3K27me2K36me3 | H3K27K36 | 1.050 | 1.018 | 0.643 | #N/A | #N/A |
| H3K27me3K36me0 | H3K27K36 | 0.209 | 0.291 | 0.081 | 0.689 | 0.037 |
| H3K27me3K36me1 | H3K27K36 | −0.214 | 0.212 | −0.178 | 0.391 | 0.170 |
| H3K27me3K36me2 | H3K27K36 | 0.236 | 0.259 | 0.066 | 0.085 | −0.151 |
| H3K27me3K36me3 | H3K27K36 | 1.439 | 1.614 | 1.393 | #N/A | #N/A |
| H3K27ac1K36me0 | H3K27K36 | 1.564 | #N/A | 1.201 | 1.481 | #N/A |
| H3K27ac1K36me1 | H3K27K36 | 1.131 | 1.759 | 1.594 | #N/A | #N/A |
| H3K27ac1K36me3 | H3K27K36 | 1.652 | 1.675 | 1.562 | 2.134 | 2.004 |
| H3K56me0 | H3K56 | 0.111 | 0.303 | −0.071 | 0.856 | 0.297 |
| H3K79me1 | H3K79 | 0.268 | 0.441 | 0.301 | 0.954 | 0.540 |
| H3K79me2 | H3K79 | 0.498 | 0.469 | 0.455 | 0.826 | 0.557 |

TABLE 6B
replace na: values from Tables 6A are copy/pasted and “#N/A” values are removed. The third to seventh columns
separate values by experiment. Columns to the right of the matrix calculate required averages and medians for subsequent analyses.

| | Histone Mark | | | | | | | |
|---|---|---|---|---|---|---|---|---|
| | | MM01_A01 | MM02_B01 | MM03_C01 | MM04_D01 | MM05_E01 | | |
| | | Cell Line | | | | | | |
| | | HEK293T | HEK293T | HEK293T | HEK293T | HEK293T | | |
| | | Perturbation | | | | | | |
| | | pull down | pull down | pull down | SSX1 | SSX1 | pulldown | Median across |
| | Parent Peptide | input | input | input | pulldown | pulldown | ctrl avg | all experiments |
| H4(4to17)ac0me0 | H4K5K8K12K1 | −0.999 | −0.768 | −0.681 | −0.613 | −0.573 | −0.816 | −0.681 |
| H4(4to17)K5ac1me0 | H4K5K8K12K1 | 0.048 | 0.332 | 0.333 | −0.053 | 0.075 | 0.237 | 0.075 |
| H4(4to17)K12ac1me0 | H4K5K8K12K1 | −0.327 | 0.036 | 0.140 | −0.207 | −0.275 | −0.050 | −0.207 |
| H4(4to17)K16ac1me0 | H4K5K8K12K1 | −0.265 | −0.168 | −0.258 | −0.391 | −0.337 | −0.230 | −0.265 |
| H4(4to17)K8ac1K12ac1me0 | H4K5K8K12K1 | −0.117 | 0.026 | 0.240 | −0.198 | −0.093 | 0.050 | −0.093 |
| H4(4to17)K5ac1K8ac1me0 | H4K5K8K12K1 | 0.502 | 0.475 | 0.651 | 0.107 | 0.094 | 0.543 | 0.475 |
| H4(4to17)K5ac1K16ac1me0 | H4K5K8K12K1 | 0.618 | 0.676 | 0.621 | 0.226 | 0.367 | 0.638 | 0.618 |
| H4(4to17)K12ac1K16ac1me | H4K5K8K12K1 | 0.411 | 0.391 | 0.424 | 0.204 | 0.304 | 0.409 | 0.391 |
| H4(4to17)K5ac1K8ac1K12ac | H4K5K8K12K1 | −0.054 | 0.091 | 0.312 | −0.223 | −0.270 | 0.116 | −0.054 |
| H4(4to17)K8ac1K12ac1K16a | H4K5K8K12K1 | 0.816 | 0.897 | 1.090 | 0.677 | 0.625 | 0.935 | 0.816 |
| H4(4to17)K5ac1K8ac1K16ac | H4K5K8K12K1 | 0.819 | 0.755 | 0.972 | 0.474 | 0.311 | 0.849 | 0.755 |
| H4(4to17)K5ac1K8ac1K12ac | H4K5K8K12K1 | 0.718 | 0.864 | 1.040 | 0.276 | 0.245 | 0.874 | 0.718 |
| H4(20to23)K20me0 | H4K20 | −0.045 | −0.175 | 0.077 | 0.017 | 0.092 | −0.048 | 0.017 |
| H4(20to23)K20me1 | H4K20 | −0.658 | −0.526 | −0.731 | −0.427 | −0.443 | −0.638 | −0.526 |
| H4(20to23)K20me2 | H4K20 | −0.424 | −0.453 | −0.437 | −0.414 | −0.391 | −0.438 | −0.424 |
| H4(20to23)K20me3 | H4K20 | 0.161 | 0.261 | 0.136 | −0.109 | 0.055 | 0.186 | 0.136 |
| H2AZ(1to19)ac0 | H2A.Z | −0.160 | −0.062 | −0.047 | −0.784 | −0.697 | −0.090 | −0.160 |
| H2AZ(1to19)K4ac1 | H2A.Z | 0.890 | 1.031 | 0.906 | −0.217 | 0.048 | 0.943 | 0.890 |
| H2B(1to29)ac0 | H2B | 0.112 | −0.187 | −0.042 | 0.152 | 0.160 | −0.039 | 0.112 |
| H2B(1to29)K5ac1 | H2B | 0.234 | 0.001 | 0.124 | 0.273 | 0.323 | 0.120 | 0.234 |
| H2A(4to11)ac0 | H2AK5K9 | −0.187 | −0.105 | 0.115 | −0.026 | 0.061 | −0.059 | −0.026 |
| H2A(4to11)K5ac1 | H2AK5K9 | 0.275 | 0.713 | 0.614 | 0.569 | 0.624 | 0.534 | 0.614 |
| H2A(4to11)K9ac1 | H2AK5K9 | −0.837 | −0.552 | −0.586 | −0.254 | −0.375 | −0.658 | −0.552 |
| H2A(4to11)K5ac1K9ac1 | H2AK5K9 | −0.224 | −0.043 | 0.025 | −0.031 | −0.049 | −0.081 | −0.043 |
| H2A(12to17)ac0 | H2AK13K15 | −0.521 | −0.203 | 0.070 | 0.201 | −0.120 | −0.218 | −0.120 |
| H2A(12to17)K13ac1 | H2AK13K15 | −0.459 | −0.129 | −0.116 | 0.032 | −0.047 | −0.234 | −0.116 |
| H2A(12to17)K15ac1 | H2AK13K15 | −0.430 | −0.070 | 0.035 | 0.042 | −0.086 | −0.155 | −0.070 |
| H3K4me0 | H3K4 | −0.049 | 0.108 | 0.047 | 0.549 | 0.339 | 0.035 | 0.108 |
| H3K4me1 | H3K4 | 0.393 | 0.542 | 0.415 | 0.740 | 0.353 | 0.450 | 0.415 |
| H3K4me2 | H3K4 | 0.848 | 0.992 | 0.801 | 0.778 | 0.532 | 0.881 | 0.801 |
| H3K4me3 | H3K4 | 0.953 | 1.142 | 0.953 | 0.766 | 0.505 | 1.016 | 0.953 |
| H3K4ac1 | H3K4 | 0.485 | 0.651 | 0.588 | 0.263 | 0.320 | 0.575 | 0.485 |
| H3K9me0K14ac0 | H3K9K14 | −0.262 | −0.323 | −0.280 | 0.274 | −0.036 | −0.288 | −0.262 |
| H3K9me1K14ac0 | H3K9K14 | −0.501 | −0.392 | −0.314 | 0.206 | 0.046 | −0.402 | −0.314 |
| H3K9me2K14ac0 | H3K9K14 | −0.585 | −0.359 | −0.568 | 0.035 | −0.284 | −0.504 | −0.359 |
| H3K9me3K14ac0 | H3K9K14 | −0.364 | −0.401 | −0.153 | 0.348 | 0.123 | −0.306 | −0.153 |
| H3K9ac1K14ac0 | H3K9K14 | 1.020 | 1.120 | 0.965 | 1.203 | 1.075 | 1.035 | 1.075 |
| H3K9me0K14ac1 | H3K9K14 | 0.271 | 0.259 | −0.018 | 0.637 | 0.469 | 0.170 | 0.271 |
| H3K9me1K14ac1 | H3K9K14 | 0.565 | 0.691 | 0.585 | 0.769 | 0.508 | 0.614 | 0.585 |
| H3K9me2K14ac1 | H3K9K14 | 0.390 | 0.458 | 0.421 | 0.724 | 0.530 | 0.423 | 0.458 |
| H3K9me3K14ac1 | H3K9K14 | 0.657 | 0.663 | 0.268 | 0.828 | 0.602 | 0.529 | 0.657 |
| H3K9ac1K14ac1 | H3K9K14 | 1.842 | 1.990 | 1.786 | 2.038 | 1.804 | 1.872 | 1.842 |
| H3K9me0S10ph1K14ac0 | H3K9K14 | −2.614 | −3.006 | −2.823 | | | −2.814 | −2.823 |
| H3K9me1S10ph1K14ac0 | H3K9K14 | −2.261 | −2.090 | −2.268 | | | −2.206 | −2.261 |
| H3K9me2S10ph1K14ac0 | H3K9K14 | −2.004 | −1.899 | −2.048 | −0.683 | −1.092 | −1.984 | −1.899 |
| H3K9me3S10ph1K14ac0 | H3K9K14 | −0.920 | −0.606 | −0.834 | −0.566 | −1.026 | −0.787 | −0.834 |
| H3K9me0S10ph1K14ac1 | H3K9K14 | −2.471 | −2.280 | −1.882 | | | −2.211 | −2.280 |
| H3K9me1S10ph1K14ac1 | H3K9K14 | −2.299 | −3.153 | −3.422 | −2.668 | −2.507 | −2.958 | −2.668 |
| H3K9me2S10ph1K14ac1 | H3K9K14 | −1.957 | −2.023 | −2.085 | −0.870 | −1.009 | −2.022 | −1.957 |
| H3K18ac0K23ac0 | H3K18K23 | −0.505 | −0.140 | −0.317 | 0.268 | 0.069 | −0.321 | −0.140 |
| H3K18ac1K23ac0 | H3K18K23 | 1.168 | 1.411 | 1.229 | 1.341 | 1.185 | 1.270 | 1.229 |
| H3K18ac0K23ac1 | H3K18K23 | 0.768 | 1.061 | 0.574 | 1.141 | 1.021 | 0.801 | 1.021 |
| H3K18ac1K23ac1 | H3K18K23 | 2.134 | 2.522 | 2.107 | 2.137 | 1.988 | 2.254 | 2.134 |
| H3K27me0K36me0 | H3K27K36 | −0.169 | 0.187 | −0.678 | 0.552 | 0.233 | −0.220 | 0.187 |
| H3K27me0K36me1 | H3K27K36 | −0.407 | −0.152 | −0.659 | 0.384 | −0.066 | −0.406 | −0.152 |
| H3K27me0K36me2 | H3K27K36 | −0.476 | −0.061 | −0.419 | 0.674 | 0.270 | −0.319 | −0.061 |
| H3K27me0K36me3 | H3K27K36 | −0.057 | −0.001 | 0.070 | 0.678 | 0.669 | 0.004 | 0.070 |
| H3K27me1K36me0 | H3K27K36 | −0.086 | 0.026 | −0.148 | 0.396 | 0.104 | −0.069 | 0.026 |
| H3K27me1K36me1 | H3K27K36 | −0.194 | 0.078 | −0.086 | 0.535 | 0.196 | −0.067 | 0.078 |
| H3K27me1K36me2 | H3K27K36 | 0.248 | 0.397 | 0.323 | 0.741 | 0.461 | 0.322 | 0.397 |
| H3K27me1K36me3 | H3K27K36 | 0.229 | 0.344 | 0.296 | 1.283 | 0.859 | 0.289 | 0.344 |
| H3K27me2K36me0 | H3K27K36 | −0.337 | −0.004 | −0.152 | 0.382 | 0.209 | −0.164 | −0.004 |
| H3K27me2K36me1 | H3K27K36 | −0.092 | −0.102 | 0.017 | 0.363 | 0.186 | −0.059 | 0.017 |
| H3K27me2K36me2 | H3K27K36 | 0.249 | 0.342 | 0.242 | 0.553 | 0.417 | 0.278 | 0.342 |
| H3K27me2K36me3 | H3K27K36 | 1.050 | 1.018 | 0.643 | | | 0.904 | 1.018 |
| H3K27me3K36me0 | H3K27K36 | 0.209 | 0.291 | 0.081 | 0.689 | 0.037 | 0.194 | 0.209 |
| H3K27me3K36me1 | H3K27K36 | −0.214 | 0.212 | −0.178 | 0.391 | 0.170 | −0.060 | 0.170 |
| H3K27me3K36me2 | H3K27K36 | 0.236 | 0.259 | 0.066 | 0.085 | −0.151 | 0.187 | 0.085 |
| H3K27me3K36me3 | H3K27K36 | 1.439 | 1.614 | 1.393 | | | 1.482 | 1.439 |
| H3K27ac1K36me0 | H3K27K36 | 1.564 | | 1.201 | 1.481 | | 1.382 | 1.481 |
| H3K27ac1K36me1 | H3K27K36 | 1.131 | 1.759 | 1.594 | | | 1.494 | 1.594 |
| H3K27ac1K36me3 | H3K27K36 | 1.652 | 1.675 | 1.562 | 2.134 | 2.004 | 1.630 | 1.675 |
| H3K56me0 | H3K56 | 0.111 | 0.303 | −0.071 | 0.856 | 0.297 | 0.114 | 0.297 |
| H3K79me1 | H3K79 | 0.268 | 0.441 | 0.301 | 0.954 | 0.540 | 0.337 | 0.441 |
| H3K79me2 | H3K79 | 0.498 | 0.469 | 0.455 | 0.826 | 0.557 | 0.474 | 0.498 |

TABLE 6C
norm to ctrl avg: normalizing experiment values to average value for control samples.

| | Histone Mark | | | | | |
|---|---|---|---|---|---|---|
| | | MM01_A01 | MM02_B01 | MM03_C01 | MM04_D01 | MM05_E01 |
| | | Cell Line | | | | |
| | | HEK293T | HEK293T | HEK293T | HEK293T | HEK293T |
| | | Perturbation | | | | |
| | | pull down | pull down | pull down | SSX1 | SSX1 |
| | Parent Peptide | input | input | input | pulldown | pulldown |
| H4(4to17)ac0me0 | H4K5K8K12K16 | −0.183 | 0.048 | 0.135 | 0.203 | 0.243 |
| H4(4to17)K5ac1me0 | H4K5K8K12K16 | −0.190 | 0.094 | 0.095 | −0.291 | −0.162 |
| H4(4to17)K12ac1me0 | H4K5K8K12K16 | −0.277 | 0.086 | 0.191 | −0.156 | −0.225 |
| H4(4to17)K16ac1me0 | H4K5K8K12K16 | −0.034 | 0.062 | −0.028 | −0.161 | −0.106 |
| H4(4to17)K8ac1K12ac1me0 | H4K5K8K12K16 | −0.166 | −0.024 | 0.190 | −0.247 | −0.143 |
| H4(4to17)K5ac1K8ac1me0 | H4K5K8K12K16 | −0.041 | −0.067 | 0.108 | −0.436 | −0.448 |
| H4(4to17)K5ac1K16ac1me0 | H4K5K8K12K16 | −0.020 | 0.038 | −0.018 | −0.412 | −0.272 |
| H4(4to17)K12ac1K16ac1me0 | H4K5K8K12K16 | 0.002 | −0.018 | 0.016 | −0.204 | −0.105 |
| H4(4to17)K5ac1K8ac1K12ac1me0 | H4K5K8K12K16 | −0.170 | −0.026 | 0.195 | −0.340 | −0.387 |
| H4(4to17)K8ac1K12ac1K16ac1me0 | H4K5K8K12K16 | −0.118 | −0.037 | 0.155 | −0.258 | −0.309 |
| H4(4to17)K5ac1K8ac1K16ac1me0 | H4K5K8K12K16 | −0.029 | −0.094 | 0.123 | −0.375 | −0.537 |
| H4(4to17)K5ac1K8ac1K12ac1K16ac1me0 | H4K5K8K12K16 | −0.156 | −0.010 | 0.166 | −0.599 | −0.629 |
| H4(20to23)K20me0 | H4K20 | 0.003 | −0.127 | 0.125 | 0.065 | 0.140 |
| H4(20to23)K20me1 | H4K20 | −0.019 | 0.112 | −0.093 | 0.211 | 0.195 |
| H4(20to23)K20me2 | H4K20 | 0.014 | −0.015 | 0.001 | 0.024 | 0.047 |
| H4(20to23)K20me3 | H4K20 | −0.025 | 0.075 | −0.050 | −0.295 | −0.131 |
| H2AZ(1to19)ac0 | H2A.Z | −0.071 | 0.028 | 0.043 | −0.694 | −0.608 |
| H2AZ(1to19)K4ac1 | H2A.Z | −0.052 | 0.089 | −0.036 | −1.159 | −0.894 |
| H2B(1to29)ac0 | H2B | 0.151 | −0.148 | −0.003 | 0.191 | 0.199 |
| H2B(1to29)K5ac1 | H2B | 0.114 | −0.118 | 0.004 | 0.154 | 0.203 |
| H2A(4to11)ac0 | H2AK5K9 | −0.128 | −0.046 | 0.174 | 0.033 | 0.120 |
| H2A(4to11)K5ac1 | H2AK5K9 | −0.259 | 0.179 | 0.080 | 0.036 | 0.090 |
| H2A(4to11)K9ac1 | H2AK5K9 | −0.179 | 0.106 | 0.072 | 0.404 | 0.283 |
| H2A(4to11)K5ac1K9ac1 | H2AK5K9 | −0.144 | 0.038 | 0.105 | 0.050 | 0.032 |
| H2A(12to17)ac0 | H2AK13K15 | −0.303 | 0.015 | 0.288 | 0.419 | 0.098 |
| H2A(12to17)K13ac1 | H2AK13K15 | −0.224 | 0.106 | 0.118 | 0.266 | 0.188 |
| H2A(12to17)K15ac1 | H2AK13K15 | −0.275 | 0.085 | 0.190 | 0.197 | 0.069 |
| H3K4me0 | H3K4 | −0.084 | 0.072 | 0.011 | 0.514 | 0.304 |
| H3K4me1 | H3K4 | −0.057 | 0.092 | −0.035 | 0.290 | −0.097 |
| H3K4me2 | H3K4 | −0.032 | 0.111 | −0.079 | −0.102 | −0.348 |
| H3K4me3 | H3K4 | −0.063 | 0.126 | −0.063 | −0.250 | −0.511 |
| H3K4ac1 | H3K4 | −0.090 | 0.076 | 0.013 | −0.312 | −0.255 |
| H3K9me0K14ac0 | H3K9K14 | 0.026 | −0.034 | 0.008 | 0.562 | 0.252 |
| H3K9me1K14ac0 | H3K9K14 | −0.099 | 0.010 | 0.088 | 0.608 | 0.449 |
| H3K9me2K14ac0 | H3K9K14 | −0.081 | 0.146 | −0.064 | 0.539 | 0.221 |
| H3K9me3K14ac0 | H3K9K14 | −0.058 | −0.095 | 0.153 | 0.654 | 0.429 |
| H3K9ac1K14ac0 | H3K9K14 | −0.015 | 0.085 | −0.070 | 0.168 | 0.040 |
| H3K9me0K14ac1 | H3K9K14 | 0.101 | 0.088 | −0.189 | 0.466 | 0.299 |
| H3K9me1K14ac1 | H3K9K14 | −0.049 | 0.077 | −0.029 | 0.155 | −0.105 |
| H3K9me2K14ac1 | H3K9K14 | −0.033 | 0.035 | −0.002 | 0.302 | 0.107 |
| H3K9me3K14ac1 | H3K9K14 | 0.128 | 0.134 | −0.261 | 0.299 | 0.072 |
| H3K9ac1K14ac1 | H3K9K14 | −0.031 | 0.117 | −0.086 | 0.165 | −0.068 |
| H3K9me0S10ph1K14ac0 | H3K9K14 | 0.200 | −0.191 | −0.009 | | |
| H3K9me1S10ph1K14ac0 | H3K9K14 | −0.055 | 0.117 | −0.062 | | |
| H3K9me2S10ph1K14ac0 | H3K9K14 | −0.020 | 0.085 | −0.064 | 1.301 | 0.892 |
| H3K9me3S10ph1K14ac0 | H3K9K14 | −0.133 | 0.181 | −0.048 | 0.221 | −0.240 |
| H3K9me0S10ph1K14ac1 | H3K9K14 | −0.260 | −0.069 | 0.329 | | |
| H3K9me1S10ph1K14ac1 | H3K9K14 | 0.659 | −0.195 | −0.464 | 0.290 | 0.451 |
| H3K9me2S10ph1K14ac1 | H3K9K14 | 0.064 | −0.001 | −0.063 | 1.152 | 1.012 |
| H3K18ac0K23ac0 | H3K18K23 | −0.184 | 0.181 | 0.004 | 0.588 | 0.390 |
| H3K18ac1K23ac0 | H3K18K23 | −0.101 | 0.141 | −0.040 | 0.072 | −0.084 |
| H3K18ac0K23ac1 | H3K18K23 | −0.033 | 0.260 | −0.227 | 0.340 | 0.220 |
| H3K18ac1K23ac1 | H3K18K23 | −0.120 | 0.267 | −0.147 | −0.117 | −0.266 |
| H3K27me0K36me0 | H3K27K36 | 0.051 | 0.407 | −0.458 | 0.773 | 0.453 |
| H3K27me0K36me1 | H3K27K36 | −0.001 | 0.254 | −0.253 | 0.790 | 0.340 |
| H3K27me0K36me2 | H3K27K36 | −0.157 | 0.257 | −0.100 | 0.993 | 0.589 |
| H3K27me0K36me3 | H3K27K36 | −0.060 | −0.005 | 0.066 | 0.674 | 0.666 |
| H3K27me1K36me0 | H3K27K36 | −0.017 | 0.095 | −0.079 | 0.465 | 0.173 |
| H3K27me1K36me1 | H3K27K36 | −0.127 | 0.145 | −0.019 | 0.602 | 0.263 |
| H3K27me1K36me2 | H3K27K36 | −0.075 | 0.074 | 0.000 | 0.419 | 0.139 |
| H3K27me1K36me3 | H3K27K36 | −0.060 | 0.054 | 0.006 | 0.993 | 0.569 |
| H3K27me2K36me0 | H3K27K36 | −0.173 | 0.161 | 0.012 | 0.546 | 0.373 |
| H3K27me2K36me1 | H3K27K36 | −0.033 | −0.043 | 0.076 | 0.421 | 0.245 |
| H3K27me2K36me2 | H3K27K36 | −0.028 | 0.064 | −0.036 | 0.276 | 0.140 |
| H3K27me2K36me3 | H3K27K36 | 0.146 | 0.115 | −0.261 | | |
| H3K27me3K36me0 | H3K27K36 | 0.015 | 0.097 | −0.112 | 0.496 | −0.157 |
| H3K27me3K36me1 | H3K27K36 | −0.154 | 0.272 | −0.118 | 0.452 | 0.230 |
| H3K27me3K36me2 | H3K27K36 | 0.049 | 0.072 | −0.121 | −0.102 | −0.338 |
| H3K27me3K36me3 | H3K27K36 | −0.043 | 0.132 | −0.089 | | |
| H3K27ac1K36me0 | H3K27K36 | 0.181 | | −0.181 | 0.099 | |
| H3K27ac1K36me1 | H3K27K36 | −0.364 | 0.264 | 0.100 | | |
| H3K27ac1K36me3 | H3K27K36 | 0.023 | 0.045 | −0.068 | 0.504 | 0.374 |
| H3K56me0 | H3K56 | −0.004 | 0.189 | −0.185 | 0.742 | 0.182 |
| H3K79me1 | H3K79 | −0.069 | 0.104 | −0.035 | 0.617 | 0.203 |
| H3K79me2 | H3K79 | 0.024 | −0.005 | −0.018 | 0.352 | 0.083 |

SSX-like protein sequences are only found in mammalian SSX family proteins (e.g., human SSX1-9) and members of the vertebrate-specific PRDM7/9 methyltransferases. A 34aa region of SSX (SSX aa155-188) that is highly conserved across vertebrate species of SSX (putative PFAM SSXRD domain) and is similar to that of PRDM7/9 proteins was identified (FIG. 3D). Pull-down experiments using biotinylated peptides corresponding to this region indicated it was sufficient for SSX nucleosome binding while shorter 23- (SSX aa166-188) and 24- (SSX aa165-188) residue peptides (lacking the W164 residue) failed to do so (FIG. 3E). This SSX-nucleosome interaction was specific as biotin pulldowns were outcompeted by addition of unlabeled SSX 34-residue peptide and could not be competed by a scrambled control peptide corresponding to the same SSX 34aa region (FIGS. 5C and 5G).

To define whether SSX 34-residue peptide can be used as a probe for repressive Barr bodies/polycomb bodies in cells, a peptide hybridization approach performed on methanol-fixed (non-crosslinked) IMR90 fibroblasts incubated with biotinylated SSX peptides and subsequently co-stained with the Barr body marker H2A K119Ub was implemented. Clear labeling of Barr bodies was observed, which indicated an innate ability of the SSX 34 residue region to selectively localize to repressed chromatin regions (FIG. 5D). 34aa regions corresponding to most human SSX proteins exhibited interactions with nucleosomes, while shorter SSX-like sequences found in PRDM7/9 proteins lacking the W164 and first R residues of the basic region (R167) failed to do so, indicating a newly evolved, mammalian-specific function of this full protein region (FIGS. 5E-5F). Finally, to identify residues important for nucleosome binding, a library of 34-residue SSX peptides containing alanine substitutions in either single conserved residues or alanine substitutions across the full basic and acidic regions was designed. Importantly, these experiments revealed that the core residues of the 6-aa basic region of the SSX (RLRERK (SEQ ID NO: 219)) were required as single residue and full region alanine substitutions in this region completely abrogated SSX-nucleosome binding (FIG. 3F).

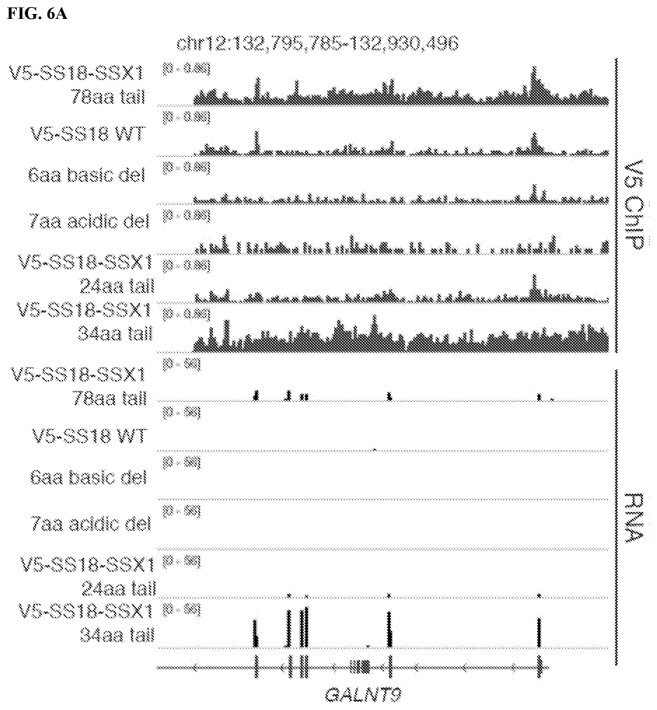

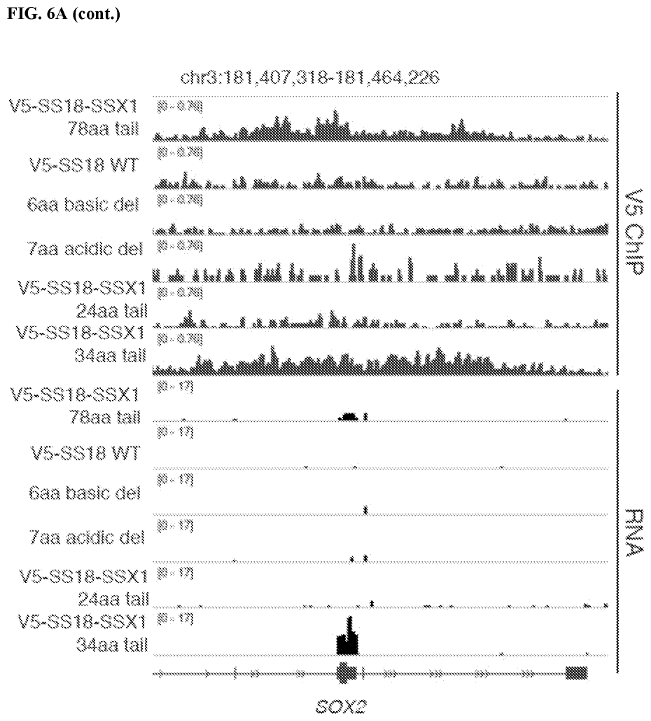

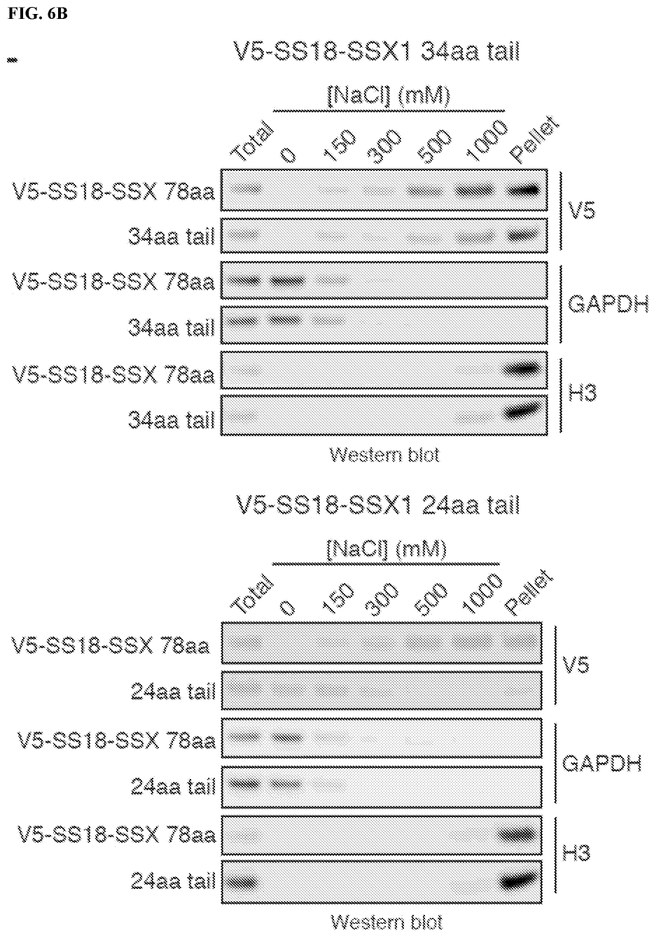

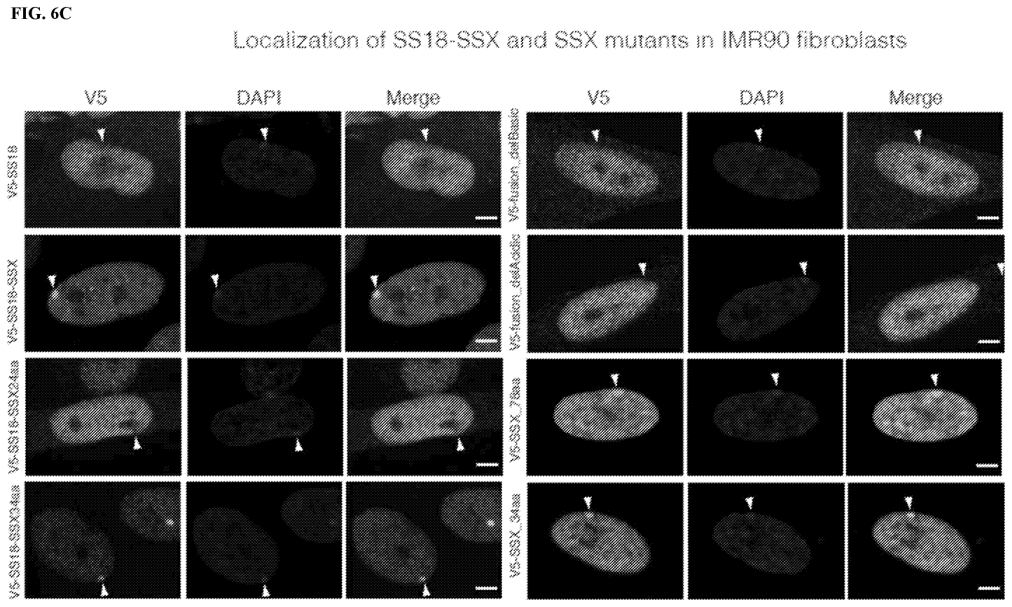

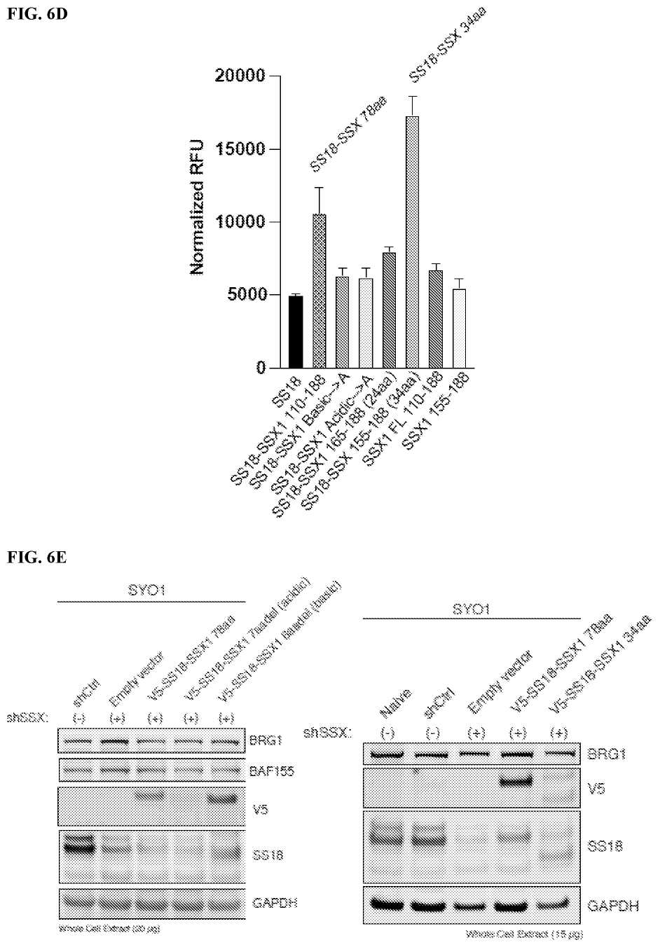

To determine whether these minimal regions were sufficient for the genome-wide targeting of fully-formed, endogenous SS18-SSX-containing BAF complexes in cells, either WT SS18, SS18-SSX, or SS18 fused to a range of mutant SSX variants for lentiviral infection in to CRL7250 human fibroblasts was expressed. ChIP-seq experiments revealed that the 34aa SSX tail fused to SS18 was sufficient to achieve SS18-SSX targeting, while the 24aa fusion was unable to do so (FIGS. 3G and 6A). Notably, deletion of either the basic or the acidic conserved regions of SSX resulted in complete loss of oncogenic fusion complex targeting, indicating that both of these regions are required for SS18-SSX-specific properties. These findings were consistent with biochemical results indicating that the full 34aa tail is needed to confer tight affinity of SS18-SSX to chromatin in cells (FIG. 6B). Importantly, these changes in chromatin targeting resulted in corresponding changes in gene expression by RNA-seq, as evidenced by clustering of the transcriptional profiles of the 34-residue tail fusion with the full SS18-SSX fusion (78-aa fusion tail), while deletion of either basic or acidic conserved regions or 24aa SSX tail variants clustered with SS18 WT gene expression profiles (FIG. 3H). These findings were further corroborated using IF for SS18-SSX Barr body localization (FIG. 6C) as well as beta-galactosidase senescence assays in IMR90 fibroblasts performed across SS18-SSX and SSX (alone) variants (FIG. 6D). Finally, both SS18-SSX-78aa and -34aa minimal fusions rescued proliferation in synovial sarcoma cell lines that are well-established to be dependent on the function of SS18-SSX and bearing shRNA-mediated KD of the endogenous SS18-SSX fusion. Taken together, these data indicate that the 34aa minimal region of SSX that contains the conserved basic and acidic regions, is responsible for the maintenance of oncogenic gene expression and proliferation in SS cell lines driven by the SS18-SSX fusion oncoprotein (FIG. 3I and FIG. 6E).

### Example 4: An RLR Motif within the SSX Basic Region Competes with SMARCB1 for Nucleosome Acidic Patch Binding, Facilitating SS18-SSX-Bound BAF Complex-Mediated Chromatin Remodeling of Polycomb-Repressed Regions

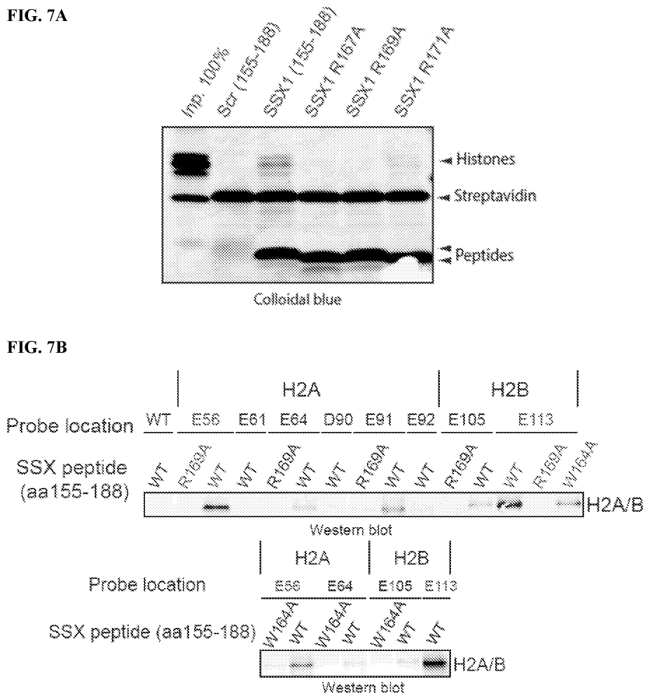

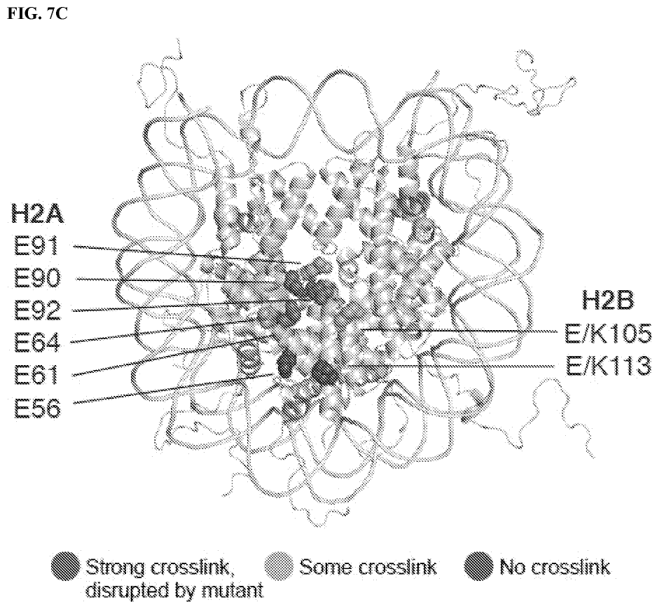

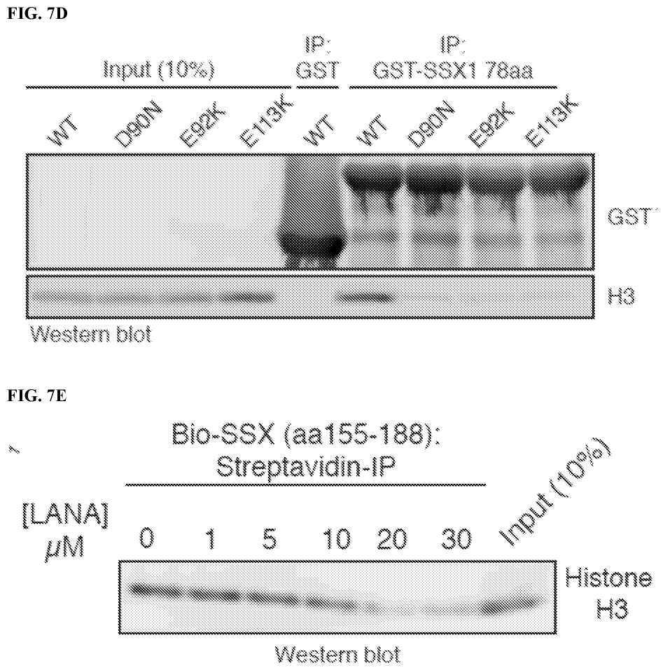

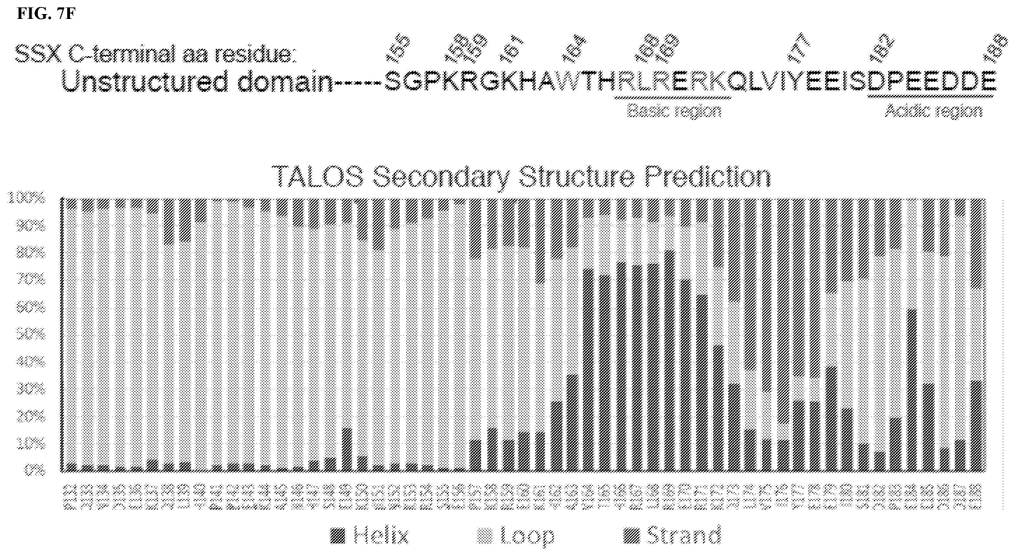

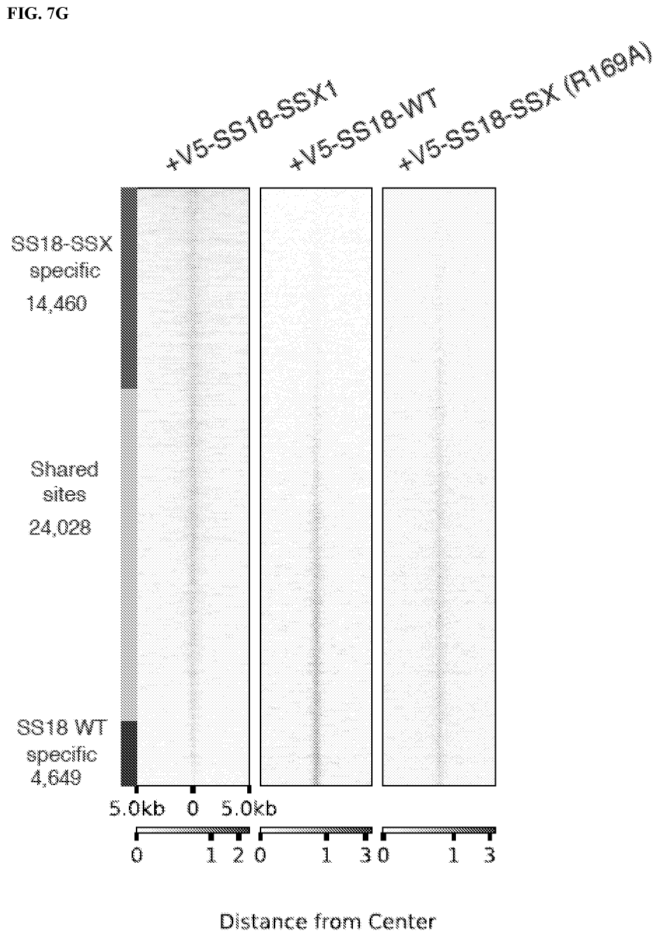

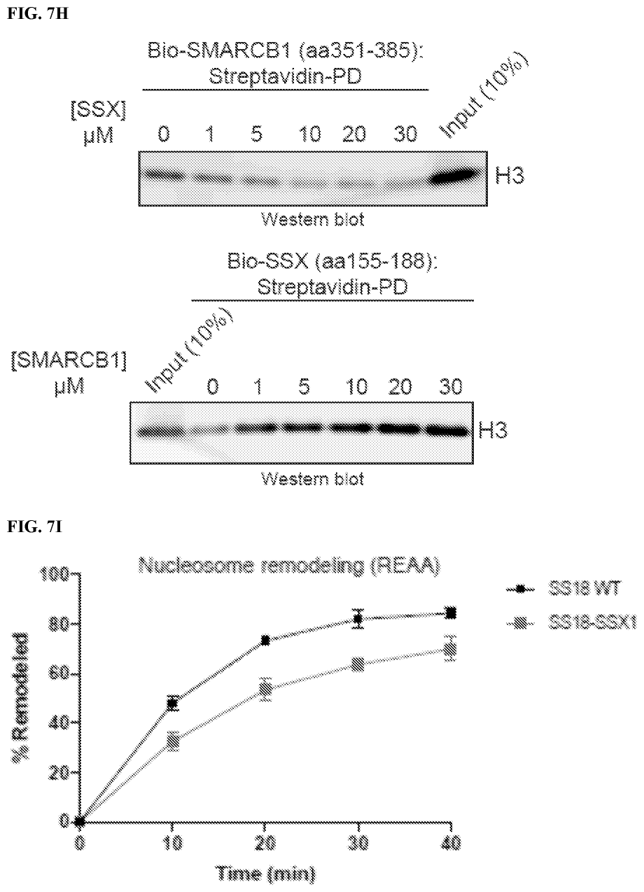

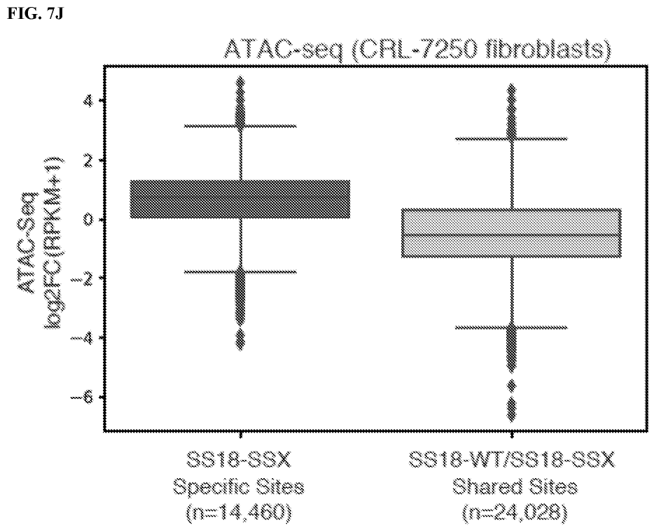

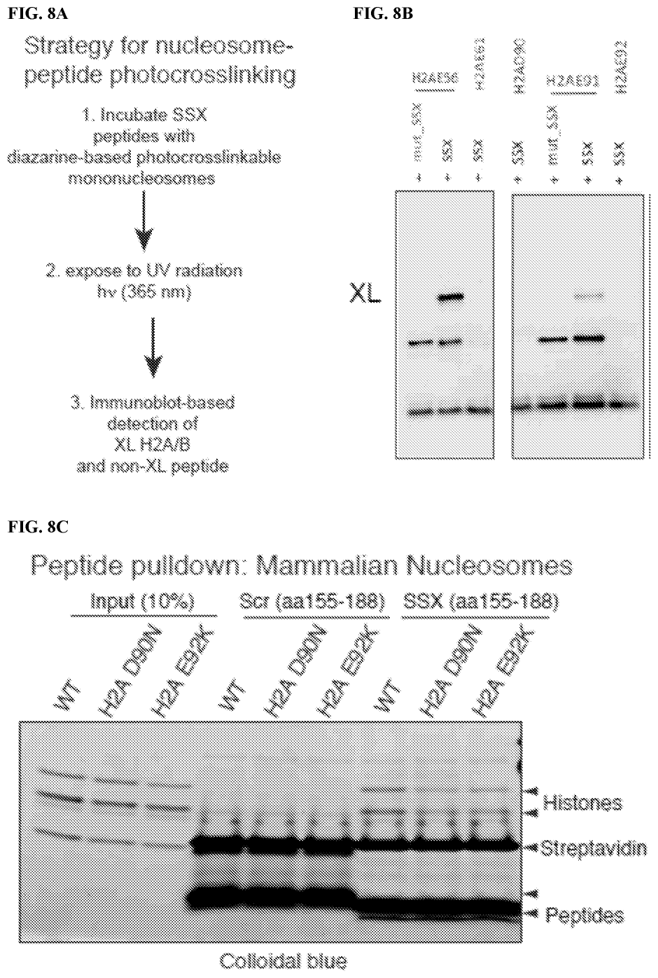

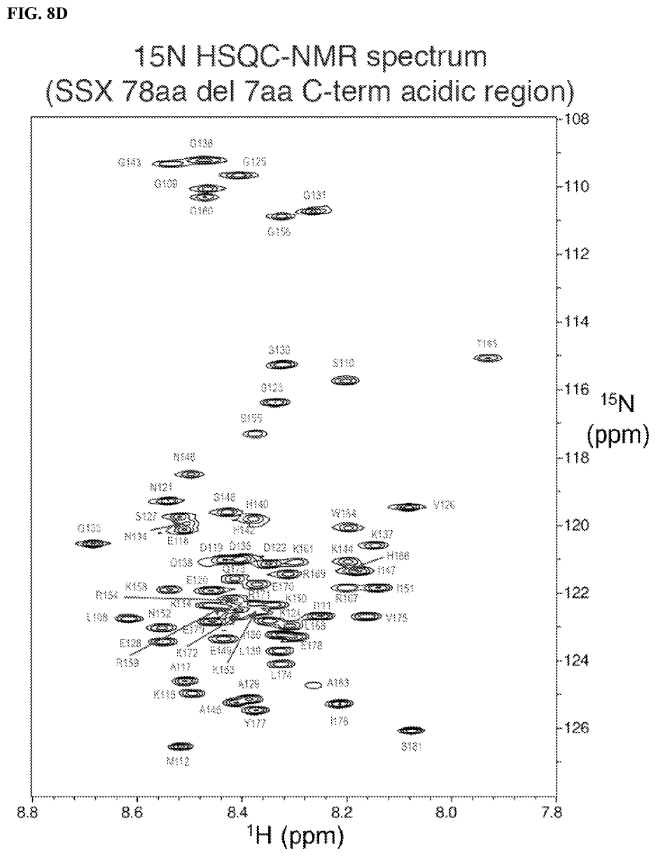

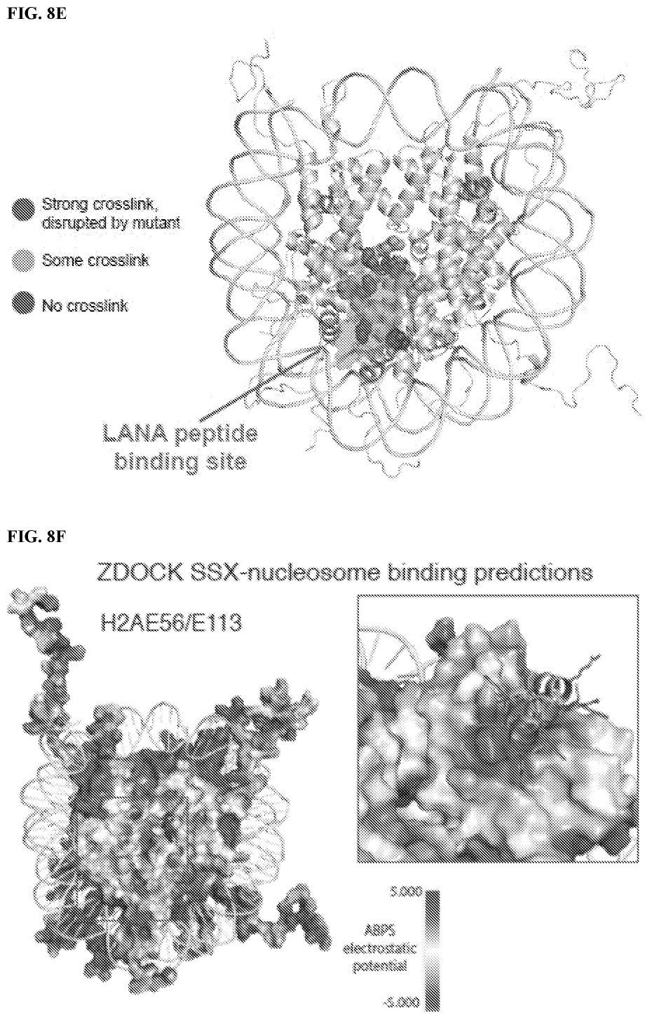

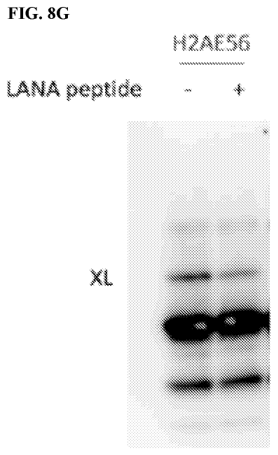

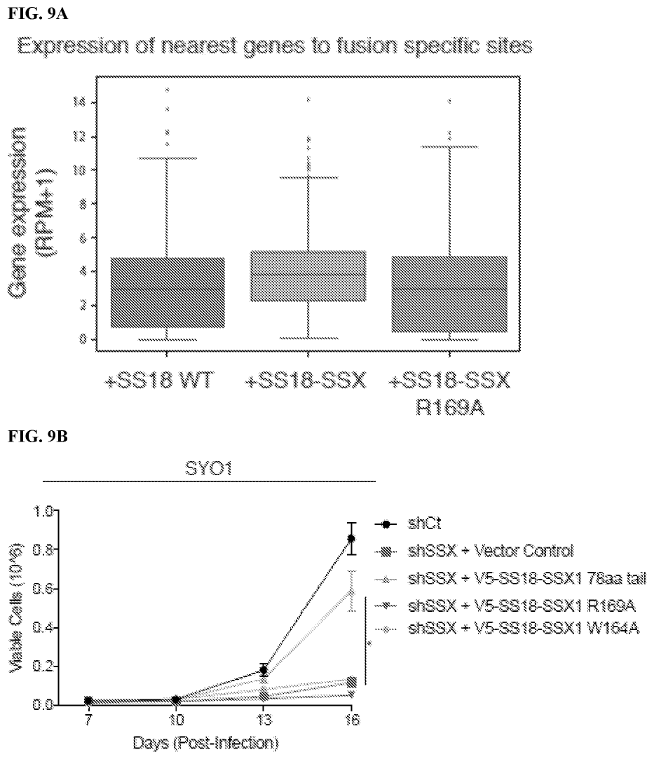

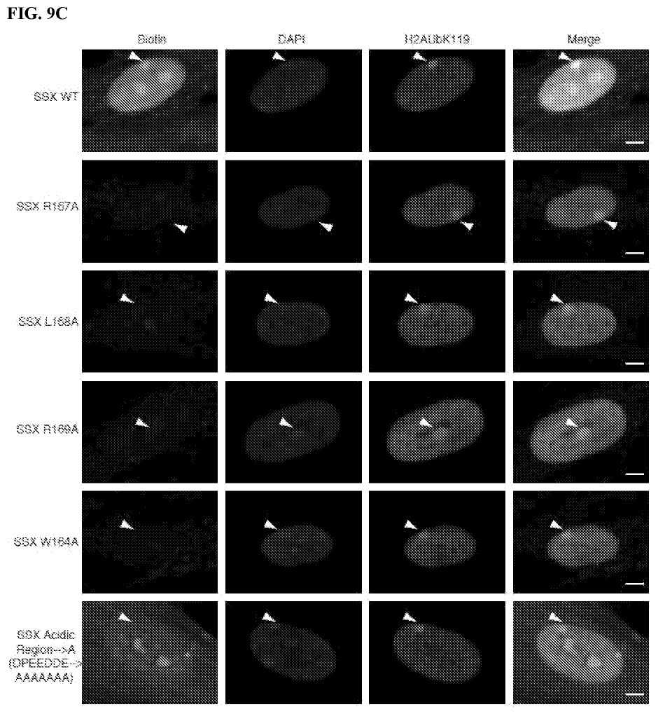

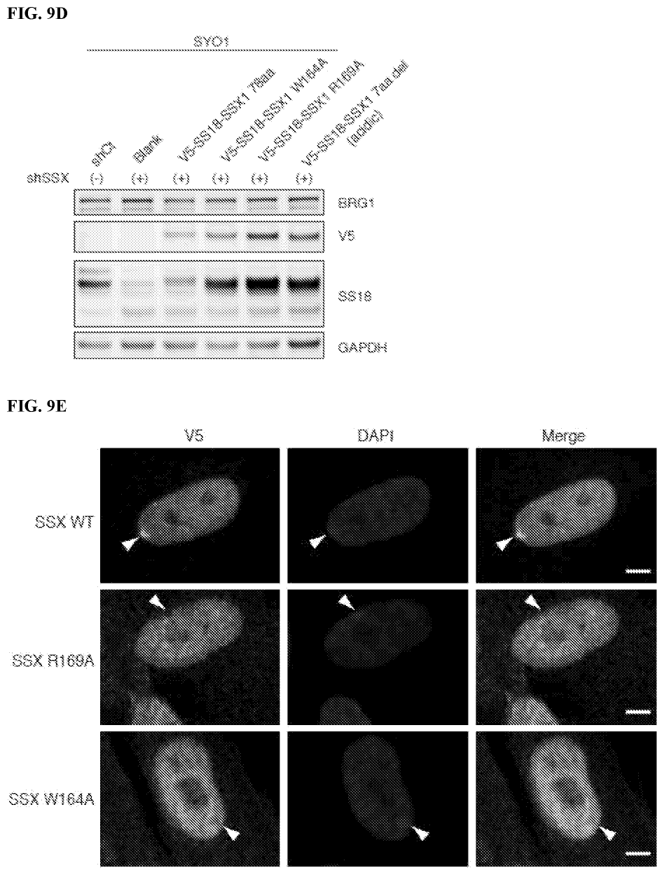

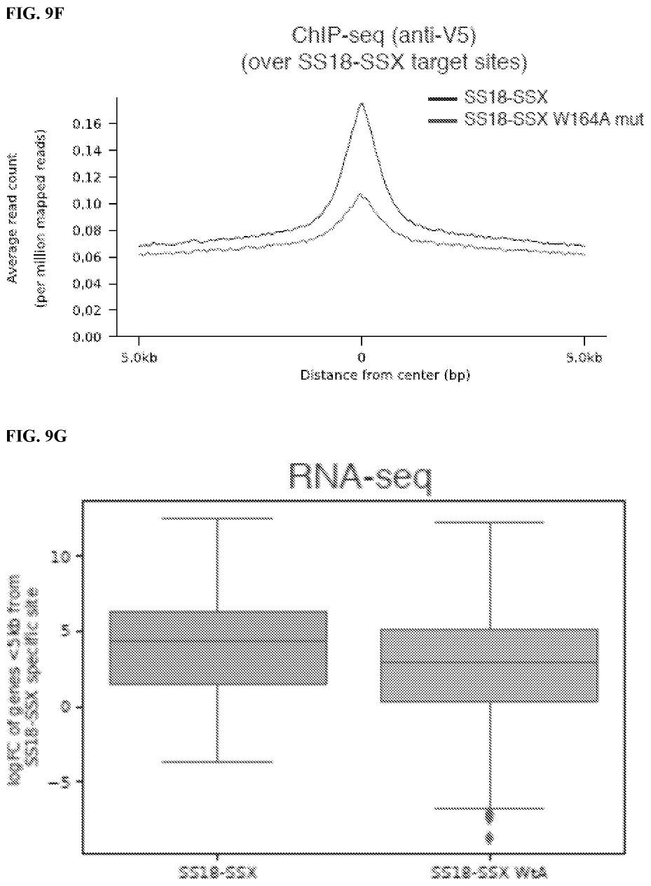

Using systematic mutagenesis on the SSX 34-residue region, it was found that single residue perturbations to the basic region, which includes a Kaposi's sarcoma-associated herpesvirus (KSHV) LANA-like RLR motif, resulted in complete loss of nucleosome binding (FIG. 7A). These data indicated that this highly basic region binds directly to the H2A/H2B acidic patch of the nucleosome. To identify the specific sites involved in acidic patch engagement, reactive diazirine probes were introduced at various residues within the nucleosome acidic patch and performed photocrosslinking studies (Dao et al. (2019) Nat. Chem. Biol. doi:10.1038/s41589-019-0413-4) with SSX 34-residue peptides (FIG. 7B and FIGS. 8A-8B). Histone-SSX crosslinks were identified at several positions across the extended acidic patch region, most prominently at positions H2A E56 and H2B E113, which importantly, were substantially reduced when key RLR basic residues in SSX were mutated (FIGS. 7B-7C). To probe this further, nucleosomes containing H2A mutant variants D90N, E92K, and E113K were assembled which disrupt the integrity of the acidic patch for GST-SSX pull down experiments. Both H2A E113 and H2B E113 are important (crosslinks were made at H2B E113) for histone-SSX interaction and mutant variants disrupt the integrity of the acidic patch demonstrating that reciprocally disrupting the integrity of the acidic patch brakes SSX binding interaction. These experiments showed near complete loss of SSX binding to acidic patch-mutant nucleosomes, indicating the importance of this highly conserved and important docking site for the SSX-chromatin interaction (FIG. 7D (homotypic) and FIG. 8C (heterotypic)). These results were further corroborated by the fact that direct nucleosome binding competition between LANA peptide and SSX was observed (FIG. 7E and FIG. 8G), as the LANA peptide is well-established to bind the nucleosome acidic patch (Barbera et al. (2006) Science 311:856-861). Notably, solution NMR studies coupled with TALOS secondary structure prediction indicated that the SSX 78aa tail protein has a disordered N-terminal region (aa 110-154) followed by a predicted alpha helical region spanning the conserved stretch of basic amino acids, WTHRLRERKQ (SEQ ID NO: 230) (FIG. 7F and FIGS. 8D-8F). In cells, single-residue mutations within the nucleosome acidic patch binding region of SSX (SSX R169A as well as W164A) resulted in attenuation of SS18-SSX-specific BAF complex chromatin occupancy, recruitment to Barr bodies, gene expression activation, and proliferative maintenance in SS cell lines (FIGS. 7G and 9A-9G). Taken together, these data establish the role for the basic region, specifically the RLR motif, in mediating SS18-SSX-nucleosome binding, in conferring SS18-SSX-containing BAF complex chromatin binding properties, as well as function in gene expression and proliferative maintenance.

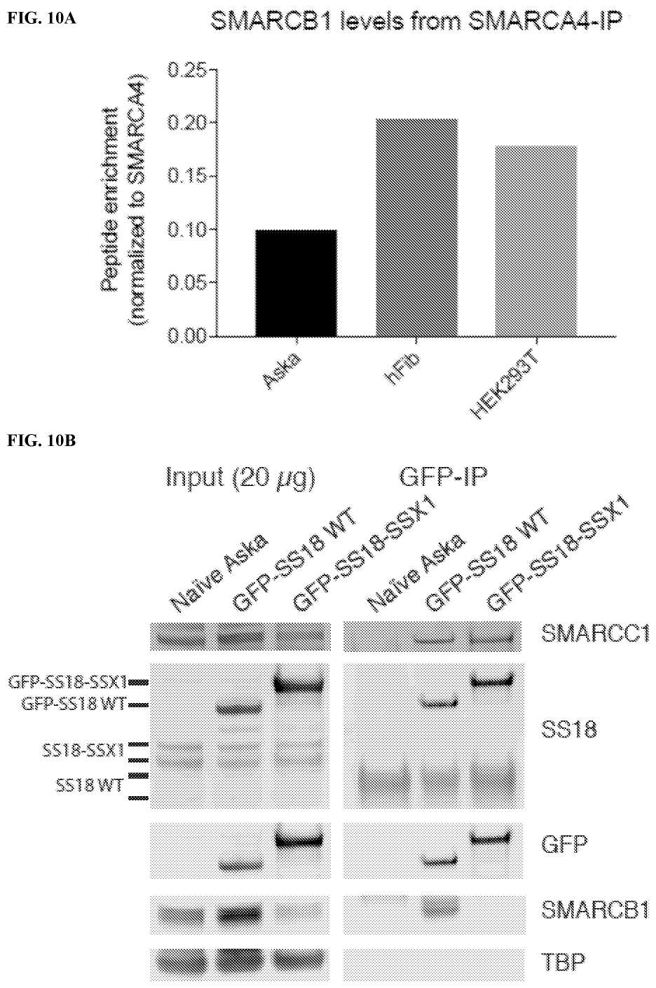

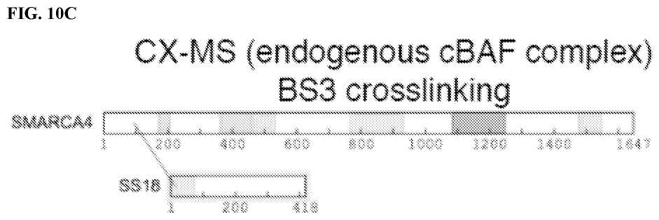

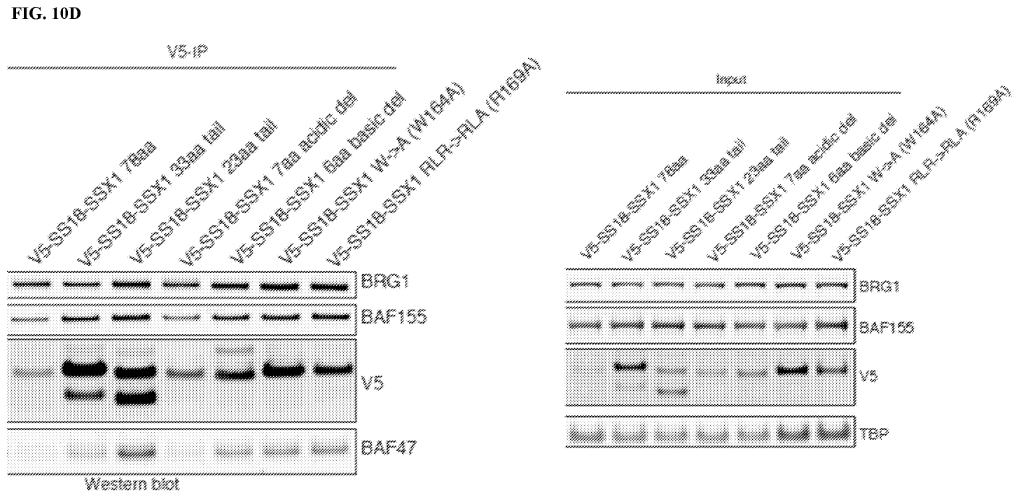

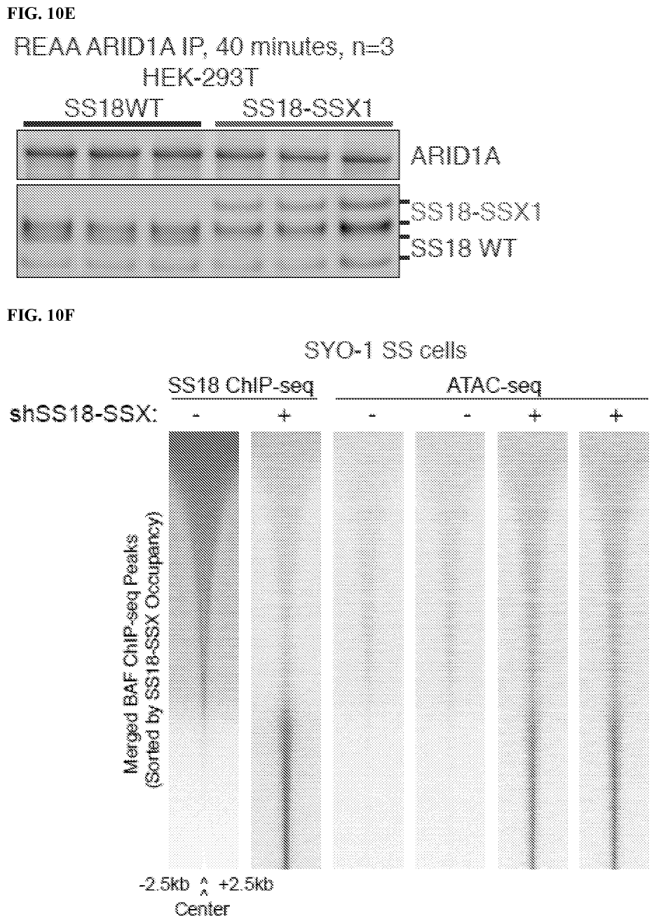

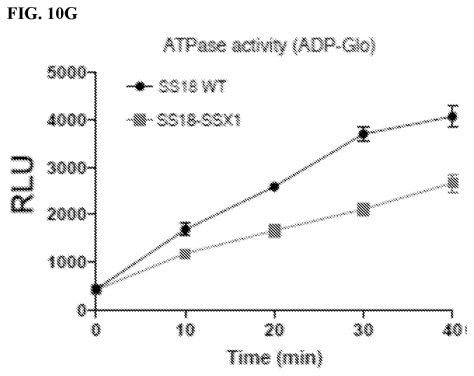

It was previously demonstrated that upon SS18-SSX expression and incorporation in to BAF complexes, the SMARCB1 (BAF47) subunit of BAF complexes, part of the core module (Mashtalir et al. (2018) Cell 175:1272-1288), is destabilized and proteasomally degraded (Kadoch and Crabtree (2013) Cell 153:71-85) (see also FIGS. 10A-10B, Tables 7A-7C). Intriguingly, using pull down competition assays, it was found that SSX competed with the recently-identified SMARCB1 C-terminal alpha helix (aa351-385) region (Valencia et al. (2019) Cell 179:1342-1356; Ye et al. (2019) Science doi:10.1126/science.aay0033) for nucleosome acidic patch binding (FIG. 7H) (Valencia et al. (2019) Cell 179:1342-1356; Ye et al. (2019) Science doi:10.1126/science.aay0033). However, the reverse was not true as the SMARCB1 C-term alpha helix was unable to outcompete SSX from binding the nucleosomes, implicating stronger affinity of SSX compared to SMARCB1 C-term for nucleosomes. This result, coupled with the positioning of SS18 at the very N-terminus SMARCA4 subunit within the core module of BAF complexes (defined by CX-MS, FIG. 10C and recent structural insights (Valencia et al. (2019) Cell 179:1342-1356; Ye et al. (2019) Science doi:10.1126/science.aay0033) and assessment of SMARCB1 levels across SS18-SSX mutant conditions (FIG. 10D) indicates the mechanism of degradation of SMARCB1 observed in SS cell lines (Kadoch and Crabtree (2013) Cell 153:71-85; McBride et al. (2018) Cancer Cell 33:1128-1141; Kohashi et al. (2010) Mod. Path. 23:981-990) can be explained by the dominant, higher affinity SSX binding to the nucleosome acidic patch and the resulting configurational changes within the BAF core module.

TABLE 7A
BAF complex components

| | Razor | | | | | | | | | |
|---|---|---|---|---|---|---|---|---|---|---|
| Protein | Peptides | Gene | Description | SeqLenn | BrgIP_Aska | BrgIP_hFib | BrgIP_293t | NSAF_BrgIP_Aska | NSAF_BrgIP_hFib | NSAF_BrgIP_293t |
| sp\|P51532\|SMCA4_HUMAN | 189 | SMARCA4 | SMCA4_HUMAN | 1647 | 50 | 39 | 100 | 0.00294971 | 0.00150089 | 0.00377919 |
| | | | Transcription | | | | | | | |
| | | | activator BRG1 | | | | | | | |
| sp\|Q92922\|SMRC1_HUMAN | 11 | SMARCC1 | SMRC1_HUMAN | 1105 | 44 | 19 | 86 | 0.00386895 | 0.00108986 | 0.00484427 |
| | | | SWI/SNF complex | | | | | | | |
| | | | subunit SMARCC1 | | | | | | | |
| sp\|Q969G3\|SMCE1_HUMAN | 96 | SMARCE1 | SMCE1_HUMAN | 411 | 39 | 21 | 36 | 0.00921988 | 0.00323859 | 0.00545196 |
| | | | SWI/SNF-related | | | | | | | |
| | | | matrix-associated | | | | | | | |
| | | | actin-dependent | | | | | | | |
| | | | regulator of | | | | | | | |
| | | | chromatin subfamily E | | | | | | | |
| | | | member 1 | | | | | | | |
| sp\|Q8TAQ2\|SMRC2_HUMAN | 136 | SMARCC2 | SMRC2_HUMAN | 1214 | 36 | 51 | 69 | 0.00288129 | 0.00266275 | 0.00353771 |
| | | | SWI/SNF complex | | | | | | | |
| | | | subunit SMARCC2 | | | | | | | |
| sp\|O14497\|ARI1A_HUMAN | 192 | ARID1A | ARI1A_HUMAN AT- | 2285 | 35 | 44 | 121 | 0.00148828 | 0.00122052 | 0.00329603 |
| | | | rich interactive | | | | | | | |
| | | | domain-containing | | | | | | | |
| | | | protein 1A | | | | | | | |
| sp\|Q8TAQ2-3\|SMRC2_HUMAN | 2 | SMARCC2 | SMRC2_HUMAN | 1152 | 33 | 50 | 55 | 0.00278333 | 0.00275103 | 0.00297168 |
| | | | Isoform 3 of SWI/SNF | | | | | | | |
| | | | complex subunit | | | | | | | |
| | | | SMARCC2 | | | | | | | |
| sp\|Q8NFD5-3\|ARI1B_HUMAN | 117 | ARID1B | ARI 1B_HUMAN | 2289 | 28 | 28 | 61 | 0.00118854 | 0.00077534 | 0.00165873 |
| | | | Isoform 3 of AT-rich | | | | | | | |
| | | | interactive domain- | | | | | | | |
| | | | containing protein 1B | | | | | | | |
| sp\|P60709\|ACTB_HUMAN | 19 | ACTB | ACTB_HUMAN Actin, | 375 | 19 | 18 | 18 | 0.00492294 | 0.00304242 | 0.00298767 |
| | | | cytoplasmic 1 | | | | | | | |
| sp\|O96019\|ACL6A_HUMAN | 55 | ACTL6A | ACL6A_HUMAN Actin- | 429 | 14 | 16 | 25 | 0.00317083 | 0.00236397 | 0.00362723 |
| | | | like protein 6A | | | | | | | |
| sp\|Q96GM5\|SMRD1_HUMAN | 30 | SMARCD1 | SMRD1_HUMAN | 515 | 14 | 6 | 18 | 0.00264133 | 0.00073845 | 0.00217549 |
| | | | SWI/SNF-related | | | | | | | |
| | | | matrix-associated | | | | | | | |
| | | | actin-dependent | | | | | | | |
| | | | regulator of | | | | | | | |
| | | | chromatin subfamily | | | | | | | |
| | | | D member 1 | | | | | | | |
| sp\|Q92925\|SMRD2_HUMAN | 42 | SMARCD2 | SMRD2_HUMAN | 531 | 12 | 9 | 21 | 0.00219578 | 0.0010743 | 0.0024616 |
| | | | SWI/SNF-related | | | | | | | |
| | | | matrix-associated | | | | | | | |
| | | | actin-dependent | | | | | | | |
| | | | regulator of | | | | | | | |
| | | | chromatin subfamily | | | | | | | |
| | | | D member 2 | | | | | | | |
| sp\|Q9NZM4\|GSCR1_HUMAN | 28 | GLTSCR1 | GSCR1_HUMAN | 1560 | 10 | 4 | 14 | 0.00062284 | 0.00016252 | 0.00055859 |
| | | | Glioma tumor | | | | | | | |
| | | | suppressor candidate | | | | | | | |
| | | | region gene 1 protein | | | | | | | |
| sp\|Q6AI39\|GSC1L_HUMAN | 0 | GLTSCR1L | GSC1L_HUMAN | 1079 | 7 | 2 | 4 | 0.00063035 | 0.00011749 | 0.00023074 |
| | | | GLTSCR 1-like protein | | | | | | | |
| sp\|Q6STE5\|SMRD3_HUMAN | 7 | SMARCD3 | SMRD3_HUMAN | 483 | 6 | 6 | 12 | 0.001207 | 0.00078738 | 0.00154641 |
| | | | SWI/SNF-related | | | | | | | |
| | | | matrix-associated | | | | | | | |
| | | | actin-dependent | | | | | | | |
| | | | regulator of | | | | | | | |
| | | | chromatin subfamily | | | | | | | |
| | | | D member 3 | | | | | | | |
| sp\|Q9H8M2\|BRD9_HUMAN | 10 | BRD9 | BRD9_HUMAN | 597 | 5 | 1 | 4 | 0.00081376 | 0.00010617 | 0.00041704 |
| | | | Bromodomain- | | | | | | | |
| | | | containing protein 9 | | | | | | | |
| sp\|Q92785\|REQU_HUMAN | 34 | DPF2 | REQU_HUMAN Zinc | 391 | 5 | 5 | 24 | 0.0012425 | 0.00081054 | 0.00382055 |
| | | | finger protein ubi-d4 | | | | | | | |
| sp\|Q12824-2\|SNF5_HUMAN | 31 | SMARCB1 | SNF5_HUMAN | 376 | 5 | 10 | 18 | 0.00129207 | 0.00134859 | 0.00297973 |
| | | | Isoform B of | | | | | | | |
| | | | SWI/SNF-related | | | | | | | |
| | | | matrix-associated | | | | | | | |
| | | | actin-dependent | | | | | | | |
| | | | regulator of | | | | | | | |
| | | | chromatin subfamily | | | | | | | |
| | | | B member 1 | | | | | | | |
| sp\|Q15532\|SSXT_HUMAN | 6 | SS18 | SSXT_HUMAN Protein | 418 | 2 | 2 | 2 | 0.0004649 | 0.00030327 | 0.00029781 |
| | | | SSXT | | | | | | | |
| sp\|Q4VC05-2\|BCL7A_HUMAN | 3 | BCL7A | BCL7A_HUMAN | 231 | 0 | 0 | 4 | 0 | 0 | 0.0010778 |
| | | | Isoform 2 of B-cell | | | | | | | |
| | | | CLL/lymphoma 7 | | | | | | | |
| | | | protein family | | | | | | | |
| | | | member A | | | | | | | |

TABLE 7B
All Spectral Counts

| | Razor | | | | | | | | | |
|---|---|---|---|---|---|---|---|---|---|---|
| Protein | Peptides | Gene | Description | SeqLen | BrgIP_Aska | BrgIP_hFib | BrgIP_293t | NSAF_BrgIP_Aska | NSAF_BrgIP_hFib | NSAF_BrgIP_293t |
| sp\|P51532\|SMCA4_HUMAN | 189 | SMARCA4 | SMCA4_HUMAN | 1647 | 50 | 39 | 100 | 0.00294971 | 0.00150089 | 0.00377919 |
| | | | Transcription activator | | | | | | | |
| | | | BRG1 | | | | | | | |
| sp\|Q92922\|SMRC1_HUMAN | 11 | SMARCC1 | SMRC1_HUMAN | 1105 | 44 | 19 | 86 | 0.00386895 | 0.00108986 | 0.00484427 |
| | | | SWI/SNF complex | | | | | | | |
| | | | subunit SMARCC1 | | | | | | | |
| sp\|Q969G3\|SMCE1_HUMAN | 96 | SMARCE1 | SMCE1_HUMAN | 411 | 39 | 21 | 36 | 0.00921988 | 0.00323859 | 0.00545196 |
| | | | SWI/SNF-related | | | | | | | |
| | | | matrix-associated | | | | | | | |
| | | | actin-dependent | | | | | | | |
| | | | regulator of chromatin | | | | | | | |
| | | | subfamily E member 1 | | | | | | | |
| sp\|P22626\|ROA2_HUMAN | 104 | HNRNPA2B1 | ROA2_HUMAN | 353 | 37 | 32 | 53 | 0.0101843 | 0.00574584 | 0.0093453 |
| | | | Heterogeneous | | | | | | | |
| | | | nuclear | | | | | | | |
| | | | ribonucleoproteins | | | | | | | |
| | | | A2/B1 | | | | | | | |
| sp\|Q8TAQ2\|SMRC2_HUMAN | 136 | SMARCC2 | SMRC2_HUMAN | 1214 | 36 | 51 | 69 | 0.00288129 | 0.00266275 | 0.00353771 |
| | | | SWI/SNF complex | | | | | | | |
| | | | subunit SMARCC2 | | | | | | | |
| sp\|O14497\|ARI1A_HUMAN | 192 | ARID1A | ARI1A_HUMAN AT-rich | 2285 | 35 | 44 | 121 | 0.00148828 | 0.00122052 | 0.00329603 |
| | | | interactive domain- | | | | | | | |
| | | | containing protein 1A | | | | | | | |
| sp\|Q8TAQ2-3\|SMRC2_HUMAN | 2 | SMARCC2 | SMRC2_HUMAN | 1152 | 33 | 50 | 55 | 0.00278333 | 0.00275103 | 0.00297168 |
| | | | Isoform 3 of SWI/SNF | | | | | | | |
| | | | complex subunit | | | | | | | |
| | | | SMARCC2 | | | | | | | |
| sp\|Q8NFD5-3\|ARI1B_HUMAN | 117 | ARID1B | ARI1B_HUMAN | 2289 | 28 | 28 | 61 | 0.00118854 | 0.000775337 | 0.00165873 |
| | | | Isoform 3 of AT-rich | | | | | | | |
| | | | interactive domain- | | | | | | | |
| | | | containing protein 1B | | | | | | | |
| sp\|P35637\|FUS_HUMAN | 53 | FUS | FUS_HUMAN RNA- | 526 | 23 | 15 | 33 | 0.00424859 | 0.00180752 | 0.00390499 |
| | | | binding protein FUS | | | | | | | |
| sp\|P78527\|PRKDC_HUMAN | 53 | PRKDC | PRKDC_HUMAN DNA- | 4128 | 23 | 13 | 22 | 0.000541366 | 0.00019961 | 0.000331722 |
| | | | dependent protein | | | | | | | |
| | | | kinase catalytic | | | | | | | |
| | | | subunit | | | | | | | |
| sp\|P60709\|ACTB_HUMAN | 19 | ACTB | ACTB_HUMAN Actin, | 375 | 19 | 18 | 18 | 0.00492294 | 0.00304242 | 0.00298767 |
| | | | cytoplasmic 1 | | | | | | | |
| sp\|P62736\|ACTA_HUMAN | 49 | ACTA2 | ACTA_HUMAN Actin, | 377 | 14 | 22 | 13 | 0.00360819 | 0.00369879 | 0.00214632 |
| | | | aortic smooth muscle | | | | | | | |
| sp\|O96019\|ACL6A_HUMAN | 55 | ACTL6A | ACL6A_HUMAN Actin- | 429 | 14 | 16 | 25 | 0.00317083 | 0.00236397 | 0.00362723 |
| | | | like protein 6A | | | | | | | |
| sp\|Q96GM5\|SMRD1_HUMAN | 30 | SMARCD1 | SMRD1_HUMAN | 515 | 14 | 6 | 18 | 0.00264133 | 0.000738452 | 0.00217549 |
| | | | SWI/SNF-related | | | | | | | |
| | | | matrix-associated | | | | | | | |
| | | | actin-dependent | | | | | | | |
| | | | regulator of chromatin | | | | | | | |
| | | | subfamily D member 1 | | | | | | | |
| sp\|P17844\|DDX5_HUMAN | 19 | DDX5 | DDX5_HUMAN | 614 | 12 | 4 | 7 | 0.00189896 | 0.000412924 | 0.000709613 |
| | | | Probable ATP- | | | | | | | |
| | | | dependent RNA | | | | | | | |
| | | | helicase DDX5 | | | | | | | |
| sp\|P02751-15\|FINC_HUMAN | 18 | FN1 | FINC_HUMAN Isoform | 2477 | 12 | 6 | 0 | 0.000470715 | 0.000153534 | 0 |
| | | | 15 of Fibronectin | | | | | | | |
| sp\|Q92925\|SMRD2_HUMAN | 42 | SMARCD2 | SMRD2_HUMAN | 531 | 12 | 9 | 21 | 0.00219578 | 0.0010743 | 0.0024616 |
| | | | SWI/SNF-related | | | | | | | |
| | | | matrix-associated | | | | | | | |
| | | | actin-dependent | | | | | | | |
| | | | regulator of chromatin | | | | | | | |
| | | | subfamily D member 2 | | | | | | | |
| sp\|P51991\|ROA3_HUMAN | 30 | HNRNPA3 | ROA3_HUMAN | 378 | 11 | 8 | 11 | 0.0028275 | 0.00134146 | 0.00181131 |
| | | | Heterogeneous | | | | | | | |
| | | | nuclear | | | | | | | |
| | | | ribonucleoprotein A3 | | | | | | | |
| sp\|Q9NZM4\|GSCR1_HUMAN | 28 | GLTSCR1 | GSCR1_HUMAN | 1560 | 10 | 4 | 14 | 0.000622842 | 0.000162523 | 0.000558593 |
| | | | Glioma tumor | | | | | | | |
| | | | suppressor candidate | | | | | | | |
| | | | region gene 1 protein | | | | | | | |
| sp\|P31942\|HNRH3_HUMAN | 54 | HNRNPH3 | HNRH3_HUMAN | 346 | 8 | 22 | 24 | 0.00224655 | 0.00403019 | 0.00431745 |
| | | | Heterogeneous | | | | | | | |
| | | | nuclear | | | | | | | |
| | | | ribonucleoprotein H3 | | | | | | | |
| sp\|Q9Y3I0\|RTCB_HUMAN | 9 | RTCB | RTCB_HUMAN tRNA- | 505 | 8 | 12 | 9 | 0.00153922 | 0.00150615 | 0.00110928 |
| | | | splicing ligase RtcB | | | | | | | |
| | | | homolog | | | | | | | |
| sp\|Q92841\|DDX17_HUMAN | 16 | DDX17 | DDX17_HUMAN | 729 | 7 | 4 | 5 | 0.000932981 | 0.000347785 | 0.000426908 |
| | | | Probable ATP- | | | | | | | |
| | | | dependent RNA | | | | | | | |
| | | | helicase DDX17 | | | | | | | |
| sp\|O00571\|DDX3X_HUMAN | 20 | DDX3X | DDX3X_HUMAN ATP- | 662 | 7 | 6 | 8 | 0.00102741 | 0.000574476 | 0.000752184 |
| | | | dependent RNA | | | | | | | |
| | | | helicase DDX3X | | | | | | | |
| sp\|Q6AI39\|GSC1L_HUMAN | 0 | GLTSCR1L | GSC1L_HUMAN | 1079 | 7 | 2 | 4 | 0.000630346 | 0.000117486 | 0.000230744 |
| | | | GLTSCR 1-like protein | | | | | | | |
| sp\|Q00839\|HNRPU_HUMAN | 33 | HNRNPU | HNRPU_HUMAN | 825 | 7 | 6 | 21 | 0.000824416 | 0.000460973 | 0.00158437 |
| | | | Heterogeneous | | | | | | | |
| | | | nuclear | | | | | | | |
| | | | ribonucleoprotein U | | | | | | | |
| sp\|Q99729-2\|ROAA_HUMAN | 17 | HNRNPAB | ROAA_HUMAN | 332 | 6 | 4 | 7 | 0.00175596 | 0.000763661 | 0.00131236 |
| | | | Isoform 2 of | | | | | | | |
| | | | Heterogeneous | | | | | | | |
| | | | nuclear | | | | | | | |
| | | | ribonucleoprotein A/B | | | | | | | |
| sp\|Q96PK6\|RBM14_HUMAN | 6 | RBM14 | RBM14_HUMAN RNA- | 669 | 6 | 11 | 14 | 0.00087142 | 0.00104219 | 0.00130255 |
| | | | binding protein 14 | | | | | | | |
| sp\|P38159\|RBMX_HUMAN | 22 | RBMX | RBMX_HUMAN RNA- | 391 | 6 | 3 | 13 | 0.001491 | 0.000486321 | 0.00206947 |
| | | | binding motif protein, | | | | | | | |
| | | | X chromosome | | | | | | | |
| sp\|Q6STE5\|SMRD3_HUMAN | 7 | SMARCD3 | SMRD3_HUMAN | 483 | 6 | 6 | 12 | 0.001207 | 0.000787377 | 0.00154641 |
| | | | SWI/SNF-related | | | | | | | |
| | | | matrix-associated | | | | | | | |
| | | | actin-dependent | | | | | | | |
| | | | regulator of chromatin | | | | | | | |
| | | | subfamily D member 3 | | | | | | | |
| sp\|Q92804\|RBP56_HUMAN | 24 | TAF15 | RBP56_HUMAN TATA- | 592 | 6 | 7 | 11 | 0.000984764 | 0.000749471 | 0.00115655 |
| | | | binding protein- | | | | | | | |
| | | | associated factor 2N | | | | | | | |
| sp\|Q9H8M2\|BRD9_HUMAN | 10 | BRD9 | BRD9_HUMAN | 597 | 5 | 1 | 4 | 0.000813763 | 0.000106171 | 0.00041704 |
| | | | Bromodomain- | | | | | | | |
| | | | containing protein 9 | | | | | | | |
| sp\|Q92785\|REQU_HUMAN | 34 | DPF2 | REQU_HUMAN Zinc | 391 | 5 | 5 | 24 | 0.0012425 | 0.000810535 | 0.00382055 |
| | | | finger protein ubi-d4 | | | | | | | |
| sp\|P09651\|ROA1_HUMAN | 9 | HNRNPA1 | ROA1_HUMAN | 372 | 5 | 4 | 9 | 0.00130596 | 0.000681546 | 0.00150588 |
| | | | Heterogeneous | | | | | | | |
| | | | nuclear | | | | | | | |
| | | | ribonucleoprotein A1 | | | | | | | |
| sp\|P11142\|HSP7C_HUMAN | 19 | HSPA8 | HSP7C_HUMAN Heat | 646 | 5 | 5 | 10 | 0.000752038 | 0.000490587 | 0.000963517 |
| | | | shock cognate 71 kDa | | | | | | | |
| | | | protein | | | | | | | |
| sp\|P38646\|GRP75_HUMAN | 12 | HSPA9 | GRP75_HUMAN | 679 | 5 | 10 | 8 | 0.000715489 | 0.000933488 | 0.000733351 |
| | | | Stress-70 protein, | | | | | | | |
| | | | mitochondrial | | | | | | | |
| sp\|Q86U86\|PB1_HUMAN | 43 | PBRM1 | PB1_HUMAN Protein | 1689 | 5 | 5 | 33 | 0.000287636 | 0.000187637 | 0.00121612 |
| | | | polybromo-1 | | | | | | | |
| sp\|Q15149\|PLEC_HUMAN | 28 | PLEC | PLEC_HUMAN Plectin | 4684 | 5 | 23 | 0 | 0.000103718 | 0.000311236 | 0 |
| sp\|Q12824-2\|SNF5_HUMAN | 31 | SMARCB1 | SNF5_HUMAN | 376 | 5 | 10 | 18 | 0.00129207 | 0.00134859 | 0.00297973 |
| | | | Isoform B of SWI/SNF- | | | | | | | |
| | | | related matrix- | | | | | | | |
| | | | associated actin- | | | | | | | |
| | | | dependent regulator | | | | | | | |
| | | | of chromatin | | | | | | | |
| | | | subfamily B member 1 | | | | | | | |
| sp\|P68371\|TBB4B_HUMAN | 5 | TUBB4B | TBB4B_HUMAN | 445 | 5 | 4 | 8 | 0.00109172 | 0.000569742 | 0.00111898 |
| | | | Tubulin beta-4B chain | | | | | | | |
| sp\|P07900-2\|HS90A_HUMAN | 10 | HSP90AA1 | HS90A_HUMAN | 854 | 4 | 4 | 2 | 0.000455098 | 0.00029688 | 0.000145769 |
| | | | Isoform 2 of Heat | | | | | | | |
| | | | shock protein HSP 90- | | | | | | | |
| | | | alpha | | | | | | | |
| sp\|P07437\|TBB5_HUMAN | 4 | TUBB | TBB5_HUMAN Tubulin | 444 | 4 | 5 | 11 | 0.000875345 | 0.000713782 | 0.00154206 |
| | | | beta chain | | | | | | | |
| sp\|Q9NWB6-2\|ARGL1_HUMAN | 4 | ARGLU1 | ARGL1_HUMAN | 273 | 3 | 0 | 1 | 0.00106773 | 0 | 0.000227997 |
| | | | Isoform 2 of Arginine | | | | | | | |
| | | | and glutamate-rich | | | | | | | |
| | | | protein 1 | | | | | | | |
| sp\|P02452\|CO1A1_HUMAN | 4 | COL1A1 | CO1A1_HUMAN | 1464 | 3 | 9 | 7 | 0.000199105 | 0.000389655 | 0.000297611 |
| | | | Collagen alpha-1(I) | | | | | | | |
| | | | chain | | | | | | | |
| sp\|P55084\|ECHB_HUMAN | 8 | HADHB | ECHB_HUMAN | 474 | 3 | 5 | 0 | 0.000614958 | 0.000668606 | 0 |
| | | | Trifunctional enzyme | | | | | | | |
| | | | subunit beta, | | | | | | | |
| | | | mitochondrial | | | | | | | |
| sp\|Q13151\|ROA0_HUMAN | 0 | HNRNPA0 | ROA0_HUMAN | 305 | 3 | 0 | 4 | 0.000955705 | 0 | 0.000816304 |
| | | | Heterogeneous | | | | | | | |
| | | | nuclear | | | | | | | |
| | | | ribonucleoprotein A0 | | | | | | | |
| sp\|Q9BUJ2\|HNRL1_HUMAN | 9 | HNRNPUL1 | HNRL1_HUMAN | 856 | 3 | 4 | 2 | 0.000340526 | 0.000296186 | 0.000145428 |
| | | | Heterogeneous | | | | | | | |
| | | | nuclear | | | | | | | |
| | | | ribonucleoprotein U- | | | | | | | |
| | | | like protein 1 | | | | | | | |
| sp\|Q12906-7\|ILF3_HUMAN | 18 | ILF3 | ILF3_HUMAN Isoform | 898 | 3 | 9 | 6 | 0.000324599 | 0.00063525 | 0.000415879 |
| | | | 7 of Interleukin | | | | | | | |
| | | | enhancer-binding | | | | | | | |
| | | | factor 3 | | | | | | | |
| sp\|Q9Y2W1\|TR150_HUMAN | 0 | THRAP3 | TR150_HUMAN | 955 | 3 | 1 | 4 | 0.000305225 | 6.63705E−05 | 0.000260704 |
| | | | Thyroid hormone | | | | | | | |
| | | | receptor-associated | | | | | | | |
| | | | protein 3 | | | | | | | |
| sp\|Q13509\|TBB3_HUMAN | 15 | TUBB3 | TBB3_HUMAN Tubulin | 450 | 3 | 5 | 7 | 0.000647756 | 0.000704265 | 0.000968228 |
| | | | beta-3 chain | | | | | | | |
| sp\|Q09666\|AHNK_HUMAN | 0 | AHNAK | AHNK_HUMAN | 5890 | 2 | 36 | 0 | 3.29926E−05 | 0.000387405 | 0 |
| | | | Neuroblast | | | | | | | |
| | | | differentiation- | | | | | | | |
| | | | associated protein | | | | | | | |
| | | | AHNAK | | | | | | | |
| sp\|P25705\|ATPA_HUMAN | 3 | ATP5A1 | ATPA_HUMAN ATP | 553 | 2 | 1 | 0 | 0.000351405 | 0.000114618 | 0 |
| | | | synthase subunit | | | | | | | |
| | | | alpha, mitochondrial | | | | | | | |
| sp\|Q9NPI1-2\|BRD7_HUMAN | 4 | BRD7 | BRD7_HUMAN Isoform | 652 | 2 | 0 | 2 | 0.000298047 | 0 | 0.00019093 |
| | | | 2 of Bromodomain- | | | | | | | |
| | | | containing protein 7 | | | | | | | |
| sp\|Q9Y224\|CN166_HUMAN | 3 | C14orf166 | CN166_HUMAN | 244 | 2 | 3 | 1 | 0.000796421 | 0.000779309 | 0.000255095 |
| | | | UPF0568 protein | | | | | | | |
| | | | C14orf166 | | | | | | | |
| sp\|Q92499\|DDX1_HUMAN | 0 | DDX1 | DDX1_HUMAN ATP- | 740 | 2 | 6 | 7 | 0.000262604 | 0.000513923 | 0.000588787 |
| | | | dependent RNA | | | | | | | |
| | | | helicase DDX1 | | | | | | | |
| sp\|Q9UJV9\|DDX41_HUMAN | 6 | DDX41 | DDX41_HUMAN | 622 | 2 | 3 | 1 | 0.000312422 | 0.00030571 | 0.000100069 |
| | | | Probable ATP- | | | | | | | |
| | | | dependent RNA | | | | | | | |
| | | | helicase DDX41 | | | | | | | |
| sp\|Q14103\|HNRPD_HUMAN | 8 | HNRNPD | HNRPD_HUMAN | 355 | 2 | 3 | 3 | 0.000547399 | 0.000535638 | 0.000525999 |
| | | | Heterogeneous | | | | | | | |
| | | | nuclear | | | | | | | |
| | | | ribonucleoprotein D0 | | | | | | | |
| sp\|O14979\|HNRDL_HUMAN | 8 | HNRNPDL | HNRDL_HUMAN | 420 | 2 | 3 | 3 | 0.000462683 | 0.000452742 | 0.000444594 |
| | | | Heterogeneous | | | | | | | |
| | | | nuclear | | | | | | | |
| | | | ribonucleoprotein D- | | | | | | | |
| | | | like | | | | | | | |
| sp\|P31943\|HNRH1_HUMAN | 11 | HNRNPH1 | HNRH1_HUMAN | 449 | 2 | 6 | 7 | 0.000432799 | 0.000847 | 0.000970384 |
| | | | Heterogeneous | | | | | | | |
| | | | nuclear | | | | | | | |
| | | | ribonucleoprotein H | | | | | | | |
| sp\|Q1KMD3\|HNRL2_HUMAN | 6 | HNRNPUL2 | HNRL2_HUMAN | 747 | 2 | 4 | 1 | 0.000260143 | 0.000339405 | 8.33242E−05 |
| | | | Heterogeneous | | | | | | | |
| | | | nuclear | | | | | | | |
| | | | ribonucleoprotein U- | | | | | | | |
| | | | like protein 2 | | | | | | | |
| sp\|P11021\|GRP78_HUMAN | 0 | HSPA5 | GRP78_HUMAN 78 | 654 | 2 | 5 | 1 | 0.000297136 | 0.000484586 | 9.51731E−05 |
| | | | kDa glucose-regulated | | | | | | | |
| | | | protein | | | | | | | |
| sp\|B9A064\|IGLL5_HUMAN | 2 | IGLL5 | IGLL5_HUMAN | 214 | 2 | 4 | 8 | 0.000908069 | 0.00118474 | 0.00232685 |
| | | | Immunoglobulin | | | | | | | |
| | | | lambda-like | | | | | | | |
| | | | polypeptide 5 | | | | | | | |
| sp\|Q13523\|PRP4B_HUMAN | 3 | PRPF4B | PRP4B_HUMAN | 1007 | 2 | 0 | 1 | 0.000192976 | 0 | 6.18105E−05 |
| | | | Serine/threonine- | | | | | | | |
| | | | protein kinase PRP4 | | | | | | | |
| | | | homolog | | | | | | | |
| sp\|P62913\|RL11_HUMAN | 5 | RPL11 | RL11_HUMAN 60S | 178 | 2 | 1 | 2 | 0.00109172 | 0.000356089 | 0.000699362 |
| | | | ribosomal protein L11 | | | | | | | |
| sp\|P40429\|RL13A_HUMAN | 5 | RPL13A | RL13A_HUMAN 60S | 203 | 2 | 1 | 2 | 0.000957274 | 0.000312236 | 0.000613233 |
| | | | ribosomal protein L13a | | | | | | | |
| sp\|P15880\|RS2_HUMAN | 6 | RPS2 | RS2_HUMAN 40S | 293 | 2 | 3 | 1 | 0.000663231 | 0.000648981 | 0.000212434 |
| | | | ribosomal protein S2 | | | | | | | |
| sp\|P05141\|ADT2_HUMAN | 0 | SLC25A5 | ADT2_HUMAN | 298 | 2 | 2 | 2 | 0.000652103 | 0.000425395 | 0.00041774 |
| | | | ADP/ATP translocase 2 | | | | | | | |
| sp\|Q15532\|SSXT_HUMAN | 6 | SS18 | SSXT_HUMAN Protein | 418 | 2 | 2 | 2 | 0.000464896 | 0.000303272 | 0.000297814 |
| | | | SSXT | | | | | | | |
| sp\|Q13263\|TIF1B_HUMAN | 5 | TRIM28 | TIF1B_HUMAN | 835 | 2 | 1 | 2 | 0.000232727 | 7.59088E−05 | 0.000149086 |
| | | | Transcription | | | | | | | |
| | | | intermediary factor 1- | | | | | | | |
| | | | beta | | | | | | | |
| sp\|Q9UHD8\|SEPT9_HUMAN | 2 | 9-Sep | SEPT9_HUMAN Septin- | 586 | 1 | 1 | 0 | 0.000165808 | 0.000108164 | 0 |
| | | | 9 | | | | | | | |
| sp\|Q68CP9\|ARID2_HUMAN | 15 | ARID2 | ARID2_HUMAN AT- | 1835 | 1 | 1 | 13 | 5.29501E−05 | 3.45416E−05 | 0.00044096 |
| | | | rich interactive | | | | | | | |
| | | | domain-containing | | | | | | | |
| | | | protein 2 | | | | | | | |
| sp\|Q8WUZ0-2\|BCL7C_HUMAN | 8 | BCL7C | BCL7C_HUMAN | 242 | 1 | 0 | 7 | 0.000401501 | 0 | 0.00180042 |
| | | | Isoform 2 of B-cell | | | | | | | |
| | | | CLL/lymphoma 7 | | | | | | | |
| | | | protein family member | | | | | | | |
| | | | C | | | | | | | |
| sp\|P02461\|CO3A1_HUMAN | 1 | COL3A1 | CO3A1_HUMAN | 1466 | 1 | 1 | 1 | 6.62779E−05 | 4.32359E−05 | 4.24578E−05 |
| | | | Collagen alpha-1(III) | | | | | | | |
| | | | chain | | | | | | | |
| sp\|P27658\|CO8A1_HUMAN | 0 | COL8A1 | CO8A1_HUMAN | 744 | 1 | 0 | 0 | 0.000130596 | 0 | 0 |
| | | | Collagen alpha-1(VIII) | | | | | | | |
| | | | chain | | | | | | | |
| sp\|Q9Y678\|COPG1_HUMAN | 2 | COPG1 | COPG1_HUMAN | 874 | 1 | 3 | 0 | 0.000111171 | 0.000217565 | 0 |
| | | | Coatomer subunit | | | | | | | |
| | | | gamma-1 | | | | | | | |
| sp\|Q8IXB1\|DJC10_HUMAN | 3 | DNAJC10 | DJC10_HUMAN DnaJ | 793 | 1 | 2 | 0 | 0.000122526 | 0.000159858 | 0 |
| | | | homolog subfamily C | | | | | | | |
| | | | member 10 | | | | | | | |
| sp\|Q5VTE0\|EF1A3_HUMAN | 6 | EEF1A1P5 | EF1A3_HUMAN | 462 | 1 | 4 | 1 | 0.00021031 | 0.000548778 | 0.000134726 |
| | | | Putative elongation | | | | | | | |
| | | | factor 1-alpha-like 3 | | | | | | | |
| sp\|P26641\|EF1G_HUMAN | 2 | EEF1G | EF1G_HUMAN | 437 | 1 | 1 | 0 | 0.000222342 | 0.000145043 | 0 |
| | | | Elongation factor 1- | | | | | | | |
| | | | gamma | | | | | | | |
| sp\|Q01844-5\|EWS_HUMAN | 8 | EWSR1 | EWS_HUMAN Isoform | 661 | 1 | 6 | 1 | 0.000146994 | 0.000575345 | 9.41652E−05 |
| | | | 5 of RNA-binding | | | | | | | |
| | | | protein EWS | | | | | | | |
| sp\|Q8NCA5\|FA98A_HUMAN | 6 | FAM98A | FA98A_HUMAN | 519 | 1 | 4 | 1 | 0.000187213 | 0.000488507 | 0.000119929 |
| | | | Protein FAM98A | | | | | | | |
| sp\|P21333\|FLNA_HUMAN | 40 | FLNA | FLNA_HUMAN Filamin- | 2647 | 1 | 39 | 0 | 0.000036707 | 0.000933876 | 0 |
| | | | A | | | | | | | |
| sp\|Q8IY81\|SPB1_HUMAN | 0 | FTSJ3 | SPB1_HUMAN pre- | 847 | 1 | 0 | 1 | 0.000114715 | 0 | 7.34867E−05 |
| | | | rRNA processing | | | | | | | |
| | | | protein FTSJ3 | | | | | | | |
| sp\|P28799\|GRN_HUMAN | 4 | GRN | GRN_HUMAN | 593 | 1 | 2 | 1 | 0.00016385 | 0.000213773 | 0.000104963 |
| | | | Granulins | | | | | | | |
| sp\|P52597\|HNRPF_HUMAN | 0 | HNRNPF | HNRPF_HUMAN | 415 | 1 | 3 | 2 | 0.000234129 | 0.000458196 | 0.000299967 |
| | | | Heterogeneous | | | | | | | |
| | | | nuclear | | | | | | | |
| | | | ribonucleoprotein F | | | | | | | |
| sp\|P08107\|HSP71_HUMAN | 5 | HSPA1A | HSP71_HUMAN Heat | 641 | 1 | 1 | 5 | 0.000151581 | 9.88827E−05 | 0.000485516 |
| | | | shock 70 kDa protein | | | | | | | |
| | | | 1A/1B | | | | | | | |
| sp\|Q07666\|KHDR1_HUMAN | 7 | KHDRBS1 | KHDR1_HUMAN KH | 443 | 1 | 1 | 5 | 0.00021933 | 0.000143079 | 0.000702519 |
| | | | domain-containing, | | | | | | | |
| | | | RNA-binding, signal | | | | | | | |
| | | | transduction- | | | | | | | |
| | | | associated protein 1 | | | | | | | |
| sp\|P56192\|SYMC_HUMAN | 8 | MARS | SYMC_HUMAN | 900 | 1 | 4 | 3 | 0.000107959 | 0.000281706 | 0.000207477 |
| | | | Methionine--tRNA | | | | | | | |
| | | | ligase, cytoplasmic | | | | | | | |
| sp\|Q9BU76\|MMTA2_HUMAN | 2 | MMTAG2 | MMTA2_HUMAN | 263 | 1 | 0 | 1 | 0.000369442 | 0 | 0.000236666 |
| | | | Multiple myeloma | | | | | | | |
| | | | tumor-associated | | | | | | | |
| | | | protein 2 | | | | | | | |
| sp\|P09874\|PARP1_HUMAN | 0 | PARP1 | PARP1_HUMAN Poly | 1014 | 1 | 2 | 1 | 9.58218E−05 | 0.000125017 | 6.13838E−05 |
| | | | [ADP-ribose] | | | | | | | |
| | | | polymerase 1 | | | | | | | |
| sp\|Q9NR12\|PDLI7_HUMAN | 16 | PDLIM7 | PDLI7_HUMAN PDZ | 457 | 1 | 15 | 0 | 0.000212611 | 0.00208043 | 0 |
| | | | and LIM domain | | | | | | | |
| | | | protein 7 | | | | | | | |
| sp\|Q8WUB8\|PHF10_HUMAN | 2 | PHF10 | PHF10_HUMAN PHD | 498 | 1 | 0 | 1 | 0.000195107 | 0 | 0.000124986 |
| | | | finger protein 10 | | | | | | | |
| sp\|Q14498\|RBM39_HUMAN | 2 | RBM39 | RBM39_HUMAN RNA- | 530 | 1 | 0 | 1 | 0.000183327 | 0 | 0.00011744 |
| | | | binding protein 39 | | | | | | | |
| sp\|P27635\|RL10_HUMAN | 2 | RPL10 | RL10_HUMAN 60S | 214 | 1 | 0 | 1 | 0.000454034 | 0 | 0.000290856 |
| | | | ribosomal protein L10 | | | | | | | |
| sp\|P18621-3\|RL17_HUMAN | 1 | RPL17 | RL17_HUMAN Isoform | 228 | 1 | 0 | 0 | 0.000426155 | 0 | 0 |
| | | | 3 of 60S ribosomal | | | | | | | |
| | | | protein L17 | | | | | | | |
| sp\|P83731\|RL24_HUMAN | 4 | RPL24 | RL24_HUMAN 60S | 157 | 1 | 1 | 2 | 0.000618875 | 0.000403719 | 0.000792907 |
| | | | ribosomal protein L24 | | | | | | | |
| sp\|P46776\|RL27A_HUMAN | 3 | RPL27A | RL27A_HUMAN 60S | 148 | 1 | 1 | 1 | 0.000656509 | 0.000428269 | 0.000420569 |
| | | | ribosomal protein L27a | | | | | | | |
| sp\|P62424\|RL7A_HUMAN | 4 | RPL7A | RL7A_HUMAN 60S | 266 | 1 | 1 | 2 | 0.000365276 | 0.000238285 | 0.000467994 |
| | | | ribosomal protein L7a | | | | | | | |
| sp\|P62269\|RS18_HUMAN | 2 | RPS18 | RS18_HUMAN 40S | 152 | 1 | 0 | 2 | 0.000639233 | 0 | 0.000818989 |
| | | | ribosomal protein S18 | | | | | | | |
| sp\|P62753\|RS6_HUMAN | 3 | RPS6 | RS6_HUMAN 40S | 249 | 1 | 1 | 1 | 0.000390214 | 0.000254554 | 0.000249973 |
| | | | ribosomal protein S6 | | | | | | | |
| sp\|Q9Y265\|RUVB1_HUMAN | 2 | RUVBL1 | RUVB1_HUMAN RuvB- | 456 | 1 | 0 | 1 | 0.000213078 | 0 | 0.000136498 |
| | | | like 1 | | | | | | | |
| sp\|Q9Y230\|RUVB2_HUMAN | 4 | RUVBL2 | RUVB2_HUMAN RuvB- | 463 | 1 | 0 | 3 | 0.000209856 | 0 | 0.000403304 |
| | | | like 2 | | | | | | | |
| sp\|P12236\|ADT3_HUMAN | 4 | SLC25A6 | ADT3_HUMAN | 298 | 1 | 2 | 1 | 0.000326051 | 0.000425395 | 0.00020887 |
| | | | ADP/ATP translocase 3 | | | | | | | |
| sp\|Q13242\|SRSF9_HUMAN | 6 | SRSF9 | SRSF9_HUMAN | 221 | 1 | 2 | 3 | 0.000439653 | 0.000573609 | 0.00084493 |
| | | | Serine/arginine-rich | | | | | | | |
| | | | splicing factor 9 | | | | | | | |
| sp\|O75177\|CREST_HUMAN | 0 | SS18L1 | CREST_HUMAN | 396 | 1 | 0 | 1 | 0.000245362 | 0 | 0.00015718 |
| | | | Calcium-responsive | | | | | | | |
| | | | transactivator | | | | | | | |
| sp\|P51571\|SSRD_HUMAN | 3 | SSR4 | SSRD_HUMAN | 173 | 1 | 3 | 0 | 0.000561638 | 0.00109914 | 0 |
| | | | Translocon-associated | | | | | | | |
| | | | protein subunit delta | | | | | | | |
| sp\|P68363\|TBA1B_HUMAN | 15 | TUBA1B | TBA1B_HUMAN | 451 | 1 | 5 | 9 | 0.00021544 | 0.000702703 | 0.0012421 |
| | | | Tubulin alpha-1B chain | | | | | | | |
| sp\|P10599\|THIO_HUMAN | 3 | TXN | THIO_HUMAN | 105 | 1 | 1 | 1 | 0.000925365 | 0.000603655 | 0.000592792 |
| | | | Thioredoxin | | | | | | | |
| sp\|Q6NZY4\|ZCHC8_HUMAN | 2 | ZCCHC8 | ZCHC8_HUMAN Zinc | 707 | 1 | 1 | 0 | 0.00013743 | 8.96518E−05 | 0 |
| | | | finger CCHC domain- | | | | | | | |
| | | | containing protein 8 | | | | | | | |
| ##sp\|Q6L8G9\|KRA56_HUMAN | 0 | ##KRTAP5-6 | ##KRA56_HUMAN | 129 | 0 | 0 | 1 | 0 | 0 | 0.000482505 |
| | | | ##Keratin-associated | | | | | | | |
| | | | protein 5-6 | | | | | | | |
| sp\|A8K2U0\|A2ML1_HUMAN | 0 | A2ML1 | A2ML1_HUMAN | 1454 | 0 | 2 | 0 | 0 | 8.71854E−05 | 0 |
| | | | Alpha-2- | | | | | | | |
| | | | macroglobulin-like | | | | | | | |
| | | | protein 1 | | | | | | | |
| sp\|P12814-4\|ACTN1_HUMAN | 2 | ACTN1 | ACTN1_HUMAN | 930 | 0 | 2 | 0 | 0 | 0.000136309 | 0 |
| | | | Isoform 4 of Alpha- | | | | | | | |
| | | | actinin-1 | | | | | | | |
| sp\|O43707\|ACTN4_HUMAN | 2 | ACTN4 | ACTN4_HUMAN | 911 | 0 | 2 | 0 | 0 | 0.000139152 | 0 |
| | | | Alpha-actinin-4 | | | | | | | |
| sp\|O95831\|AIFM1_HUMAN | 2 | AIFM1 | AIFM1_HUMAN | 613 | 0 | 1 | 1 | 0 | 0.000103399 | 0.000101539 |
| | | | Apoptosis-inducing | | | | | | | |
| | | | factor 1, mitochondrial | | | | | | | |
| sp\|Q86V81\|THOC4_HUMAN | 1 | ALYREF | THOC4_HUMAN THO | 257 | 0 | 0 | 1 | 0 | 0 | 0.000242191 |
| | | | complex subunit 4 | | | | | | | |
| sp\|P15144\|AMPN_HUMAN | 2 | ANPEP | AMPN_HUMAN | 967 | 0 | 6 | 0 | 0 | 0.000393281 | 0 |
| | | | Aminopeptidase N | | | | | | | |
| sp\|P06576\|ATPB_HUMAN | 1 | ATP5B | ATPB_HUMAN ATP | 529 | 0 | 1 | 0 | 0 | 0.000119818 | 0 |
| | | | synthase subunit beta, | | | | | | | |
| | | | mitochondrial | | | | | | | |
| sp\|Q4VC05-2\|BCL7A_HUMAN | 3 | BCL7A | BCL7A_HUMAN | 231 | 0 | 0 | 4 | 0 | 0 | 0.0010778 |
| | | | Isoform 2 of B-cell | | | | | | | |
| | | | CLL/lymphoma 7 | | | | | | | |
| | | | protein family member | | | | | | | |
| | | | A | | | | | | | |
| sp\|Q9NYF8\|BCLF1_HUMAN | 3 | BCLAF1 | BCLF1_HUMAN Bcl-2- | 920 | 0 | 0 | 3 | 0 | 0 | 0.000202967 |
| | | | associated | | | | | | | |
| | | | transcription factor 1 | | | | | | | |
| sp\|Q96MY1\|CT112_HUMAN | 1 | C20orf112 | CT112_HUMAN | 436 | 0 | 1 | 0 | 0 | 0.000145376 | 0 |
| | | | Uncharacterized | | | | | | | |
| | | | protein C20orf112 | | | | | | | |
| sp\|Q05682\|CALD1_HUMAN | 4 | CALD1 | CALD1_HUMAN | 793 | 0 | 4 | 0 | 0 | 0.000319717 | 0 |
| | | | Caldesmon | | | | | | | |
| sp\|O43852-3\|CALU_HUMAN | 1 | CALU | CALU_HUMAN Isoform | 323 | 0 | 1 | 0 | 0 | 0.000196235 | 0 |
| | | | 3 of Calumenin | | | | | | | |
| sp\|Q14444\|CAPR1_HUMAN | 1 | CAPRIN1 | CAPR1_HUMAN | 709 | 0 | 0 | 1 | 0 | 0 | 8.77901E−05 |
| | | | Caprin-1 | | | | | | | |
| sp\|Q76M96-2\|CCD80_HUMAN | 3 | CCDC80 | CCD80_HUMAN | 961 | 0 | 3 | 0 | 0 | 0.000197868 | 0 |
| | | | Isoform 2 of Coiled-coil | | | | | | | |
| | | | domain-containing | | | | | | | |
| | | | protein 80 | | | | | | | |
| sp\|Q15517\|CDSN_HUMAN | 3 | CDSN | CDSN_HUMAN | 529 | 0 | 3 | 0 | 0 | 0.000359455 | 0 |
| | | | Corneodesmosin | | | | | | | |
| sp\|Q07065\|CKAP4_HUMAN | 0 | CKAP4 | CKAP4_HUMAN | 602 | 0 | 5 | 0 | 0 | 0.000526444 | 0 |
| | | | Cytoskeleton- | | | | | | | |
| | | | associated protein 4 | | | | | | | |
| sp\|Q00610\|CLH1_HUMAN | 2 | CLTC | CLH1_HUMAN Clathrin | 1675 | 0 | 2 | 0 | 0 | 7.56822E−05 | 0 |
| | | | heavy chain 1 | | | | | | | |
| sp\|P51911\|CNN1_HUMAN | 2 | CNN1 | CNN1_HUMAN | 297 | 0 | 2 | 0 | 0 | 0.000426827 | 0 |
| | | | Calponin-1 | | | | | | | |
| sp\|Q15417\|CNN3_HUMAN | 3 | CNN3 | CNN3_HUMAN | 329 | 0 | 3 | 0 | 0 | 0.000577968 | 0 |
| | | | Calponin-3 | | | | | | | |
| sp\|Q99715\|COCA1_HUMAN | 8 | COL12A1 | COCA1_HUMAN | 3063 | 0 | 8 | 0 | 0 | 0.000165547 | 0 |
| | | | Collagen alpha-1(XII) | | | | | | | |
| | | | chain | | | | | | | |
| sp\|P08123\|CO1A2_HUMAN | 0 | COL1A2 | CO1A2_HUMAN | 1366 | 0 | 1 | 4 | 0 | 0.000046401 | 0.000182264 |
| | | | Collagen alpha-2(I) | | | | | | | |
| | | | chain | | | | | | | |
| sp\|P53618\|COPB_HUMAN | 4 | COPB1 | COPB_HUMAN | 953 | 0 | 7 | 0 | 0 | 0.000465568 | 0 |
| | | | Coatomer subunit beta | | | | | | | |
| sp\|Q9BZJ0\|CRNL1_HUMAN | 1 | CRNKL1 | CRNL1_HUMAN | 848 | 0 | 0 | 1 | 0 | 0 | 0.0000734 |
| | | | Crooked neck-like | | | | | | | |
| | | | protein 1 | | | | | | | |
| sp\|O00622\|CYR61_HUMAN | 0 | CYR61 | CYR61_HUMAN | 381 | 0 | 1 | 0 | 0 | 0.000166362 | 0 |
| | | | Protein CYR61 | | | | | | | |
| sp\|Q16531\|DDB1_HUMAN | 2 | DDB1 | DDB1_HUMAN DNA | 1140 | 0 | 2 | 0 | 0 | 0.0001112 | 0 |
| | | | damage-binding | | | | | | | |
| | | | protein 1 | | | | | | | |
| sp\|O43143\|DHX15_HUMAN | 0 | DHX15 | DHX15_HUMAN | 795 | 0 | 0 | 2 | 0 | 0 | 0.000156587 |
| | | | Putative pre-mRNA- | | | | | | | |
| | | | splicing factor ATP- | | | | | | | |
| | | | dependent RNA | | | | | | | |
| | | | helicase DHX15 | | | | | | | |
| sp\|Q08211\|DHX9_HUMAN | 1 | DHX9 | DHX9_HUMAN ATP- | 1270 | 0 | 0 | 7 | 0 | 0 | 0.000343073 |
| | | | dependent RNA | | | | | | | |
| | | | helicase A | | | | | | | |
| sp\|Q14204\|DYHC1_HUMAN | 0 | DYNC1H1 | DYHC1_HUMAN | 4646 | 0 | 3 | 0 | 0 | 0.000040928 | 0 |
| | | | Cytoplasmic dynein 1 | | | | | | | |
| | | | heavy chain 1 | | | | | | | |
| sp\|Q99848\|EBP2_HUMAN | 1 | EBNA1BP2 | EBP2_HUMAN | 306 | 0 | 0 | 1 | 0 | 0 | 0.000203409 |
| | | | Probable rRNA- | | | | | | | |
| | | | processing protein | | | | | | | |
| | | | EBP2 | | | | | | | |
| sp\|P00533\|EGFR_HUMAN | 1 | EGFR | EGFR_HUMAN | 1210 | 0 | 1 | 0 | 0 | 5.23833E−05 | 0 |
| | | | Epidermal growth | | | | | | | |
| | | | factor receptor | | | | | | | |
| sp\|P50402\|EMD_HUMAN | 1 | EMD | EMD_HUMAN Emerin | 254 | 0 | 1 | 0 | 0 | 0.000249543 | 0 |
| sp\|PO7814\|SYEP_HUMAN | 0 | EPRS | SYEP_HUMAN | 1512 | 0 | 2 | 1 | 0 | 0.000083841 | 4.11661E−05 |
| | | | Bifunctional | | | | | | | |
| | | | glutamate/proline-- | | | | | | | |
| | | | tRNA ligase | | | | | | | |
| sp\|Q9NZB2-6\|F120A_HUMAN | 4 | FAM120A | F120A_HUMAN | 1146 | 0 | 4 | 0 | 0 | 0.000221235 | 0 |
| | | | Isoform F of | | | | | | | |
| | | | Constitutive | | | | | | | |
| | | | coactivator of PPAR- | | | | | | | |
| | | | gamma-like protein 1 | | | | | | | |
| sp\|Q8WVX9\|FACR1_HUMAN | 1 | FAR1 | FACR1_HUMAN Fatty | 515 | 0 | 1 | 0 | 0 | 0.000123075 | 0 |
| | | | acyl-CoA reductase 1 | | | | | | | |
| sp\|Q9Y4F1-2\|FARP1_HUMAN | 3 | FARP1 | FARP1_HUMAN | 1076 | 0 | 3 | 0 | 0 | 0.000176721 | 0 |
| | | | Isoform 2 of FERM, | | | | | | | |
| | | | RhoGEF and pleckstrin | | | | | | | |
| | | | domain-containing | | | | | | | |
| | | | protein 1 | | | | | | | |
| sp\|P22087\|FBRL_HUMAN | 2 | FBL | FBRL_HUMAN rRNA 2′- | 321 | 0 | 0 | 2 | 0 | 0 | 0.000387808 |
| | | | O-methyltransferase | | | | | | | |
| | | | fibrillarin | | | | | | | |
| sp\|Q14315\|FLNC_HUMAN | 9 | FLNC | FLNC_HUMAN Filamin- | 2725 | 0 | 9 | 0 | 0 | 0.000209341 | 0 |
| | | | C | | | | | | | |
| sp\|P04899-4\|GNAI2_HUMAN | 9 | GNAI2 | GNAI2_HUMAN | 366 | 0 | 9 | 0 | 0 | 0.00155862 | 0 |
| | | | Isoform sGi2 of | | | | | | | |
| | | | Guanine nucleotide- | | | | | | | |
| | | | binding protein G(i) | | | | | | | |
| | | | subunit alpha-2 | | | | | | | |
| sp\|Q9BVP2\|GNL3_HUMAN | 3 | GNL3 | GNL3_HUMAN | 549 | 0 | 0 | 3 | 0 | 0 | 0.000340127 |
| | | | Guanine nucleotide- | | | | | | | |
| | | | binding protein-like 3 | | | | | | | |
| sp\|P62805\|H4_HUMAN | 0 | HIST1H4A | H4_HUMAN Histone | 103 | 0 | 1 | 0 | 0 | 0.000615377 | 0 |
| | | | H4 | | | | | | | |
| sp\|P55795\|HNRH2_HUMAN | 4 | HNRNPH2 | HNRH2_HUMAN | 449 | 0 | 3 | 3 | 0 | 0.0004235 | 0.000415879 |
| | | | Heterogeneous | | | | | | | |
| | | | nuclear | | | | | | | |
| | | | ribonucleoprotein H2 | | | | | | | |
| sp\|P61978-2\|HNRPK_HUMAN | 1 | HNRNPK | HNRPK_HUMAN | 464 | 0 | 0 | 1 | 0 | 0 | 0.000134145 |
| | | | Isoform 2 of | | | | | | | |
| | | | Heterogeneous | | | | | | | |
| | | | nuclear | | | | | | | |
| | | | ribonucleoprotein K | | | | | | | |
| sp\|P14866\|HNRPL_HUMAN | 1 | HNRNPL | HNRPL_HUMAN | 589 | 0 | 0 | 1 | 0 | 0 | 0.000105676 |
| | | | Heterogeneous | | | | | | | |
| | | | nuclear | | | | | | | |
| | | | ribonucleoprotein L | | | | | | | |
| sp\|P08238\|HS90B_HUMAN | 2 | HSP90AB1 | HS90B_HUMAN Heat | 724 | 0 | 1 | 1 | 0 | 8.75467E−05 | 8.59713E−05 |
| | | | shock protein HSP 90- | | | | | | | |
| | | | beta | | | | | | | |
| sp\|P01857\|IGHG1_HUMAN | 0 | IGHG1 | IGHG1_HUMAN Ig | 330 | 0 | 1 | 1 | 0 | 0.000192072 | 0.000188616 |
| | | | gamma-1 chain C | | | | | | | |
| | | | region | | | | | | | |
| sp\|Q12905\|ILF2_HUMAN | 0 | ILF2 | ILF2_HUMAN | 390 | 0 | 0 | 1 | 0 | 0 | 0.000159598 |
| | | | Interleukin enhancer- | | | | | | | |
| | | | binding factor 2 | | | | | | | |
| sp\|Q9H0H0\|INT2_HUMAN | 1 | INTS2 | INT2_HUMAN | 1204 | 0 | 0 | 1 | 0 | 0 | 0.000051697 |
| | | | Integrator complex | | | | | | | |
| | | | subunit 2 | | | | | | | |
| sp\|Q9NVH2\|INT7_HUMAN | 2 | INTS7 | INT7_HUMAN | 962 | 0 | 0 | 2 | 0 | 0 | 0.000129404 |
| | | | Integrator complex | | | | | | | |
| | | | subunit 7 | | | | | | | |
| sp\|Q9H1B7\|I2BPL_HUMAN | 0 | IRF2BPL | I2BPL_HUMAN | 796 | 0 | 1 | 0 | 0 | 7.96279E−05 | 0 |
| | | | Interferon regulatory | | | | | | | |
| | | | factor 2-binding | | | | | | | |
| | | | protein-like | | | | | | | |
| sp\|Q63ZY3-2\|KANK2_HUMAN | 2 | KANK2 | KANK2_HUMAN | 859 | 0 | 2 | 0 | 0 | 0.000147576 | 0 |
| | | | Isoform 2 of KN motif | | | | | | | |
| | | | and ankyrin repeat | | | | | | | |
| | | | domain-containing | | | | | | | |
| | | | protein 2 | | | | | | | |
| sp\|P52294\|IMA5_HUMAN | 1 | KPNA1 | IMA5_HUMAN | 538 | 0 | 1 | 0 | 0 | 0.000117814 | 0 |
| | | | Importin subunit | | | | | | | |
| | | | alpha-5 | | | | | | | |
| sp\|P52292\|IMA1_HUMAN | 1 | KPNA2 | IMA1_HUMAN | 529 | 0 | 0 | 2 | 0 | 0 | 0.000235324 |
| | | | Importin subunit | | | | | | | |
| | | | alpha-1 | | | | | | | |
| sp\|Q32P28-3\|P3H1_HUMAN | 3 | LEPRE1 | P3H1_HUMAN Isoform | 804 | 0 | 3 | 0 | 0 | 0.000236507 | 0 |
| | | | 3 of Prolyl 3- | | | | | | | |
| | | | hydroxylase 1 | | | | | | | |
| sp\|P09382\|LEG1_HUMAN | 0 | LGALS1 | LEG1_HUMAN | 135 | 0 | 2 | 0 | 0 | 0.00093902 | 0 |
| | | | Galectin-1 | | | | | | | |
| sp\|PO2545\|LMNA_HUMAN | 6 | LMNA | LMNA_HUMAN | 664 | 0 | 6 | 0 | 0 | 0.000572745 | 0 |
| | | | Prelamin-A/C | | | | | | | |
| sp\|Q8WWI1\|LMO7_HUMAN | 3 | LMO7 | LMO7_HUMAN LIM | 1683 | 0 | 3 | 0 | 0 | 0.000112984 | 0 |
| | | | domain only protein 7 | | | | | | | |
| sp\|P46821\|MAP1B_HUMAN | 0 | MAP1B | MAP1B_HUMAN | 2468 | 0 | 4 | 0 | 0 | 0.000102729 | 0 |
| | | | Microtubule- | | | | | | | |
| | | | associated protein 1B | | | | | | | |
| sp\|P27816\|MAP4_HUMAN | 1 | MAP4 | MAP4_HUMAN | 1152 | 0 | 1 | 0 | 0 | 5.50207E−05 | 0 |
| | | | Microtubule- | | | | | | | |
| | | | associated protein 4 | | | | | | | |
| sp\|P33993\|MCM7_HUMAN | 3 | MCM7 | MCM7_HUMAN DNA | 719 | 0 | 0 | 3 | 0 | 0 | 0.000259707 |
| | | | replication licensing | | | | | | | |
| | | | factor MCM7 | | | | | | | |
| sp\|P52701\|MSH6_HUMAN | 2 | MSH6 | MSH6_HUMAN DNA | 1360 | 0 | 0 | 2 | 0 | 0 | 9.15341E−05 |
| | | | mismatch repair | | | | | | | |
| | | | protein Msh6 | | | | | | | |
| sp\|P35579\|MYH9_HUMAN | 4 | MYH9 | MYH9_HUMAN | 1960 | 0 | 6 | 0 | 0 | 0.000194032 | 0 |
| | | | Myosin-9 | | | | | | | |
| sp\|Q969V3\|NCLN_HUMAN | 1 | NCLN | NCLN_HUMAN Nicalin | 563 | 0 | 1 | 0 | 0 | 0.000112582 | 0 |
| sp\|O15226-2\|NKRF_HUMAN | 3 | NKRF | NKRF_HUMAN Isoform | 705 | 0 | 0 | 3 | 0 | 0 | 0.000264865 |
| | | | 2 of NF-kappa-B- | | | | | | | |
| | | | repressing factor | | | | | | | |
| sp\|O95478\|NSA2_HUMAN | 0 | NSA2 | NSA2_HUMAN | 260 | 0 | 0 | 1 | 0 | 0 | 0.000239397 |
| | | | Ribosome biogenesis | | | | | | | |
| | | | protein NSA2 homolog | | | | | | | |
| sp\|P21589\|5NTD_HUMAN | 7 | NT5E | 5NTD_HUMAN 5′- | 574 | 0 | 7 | 0 | 0 | 0.000772973 | 0 |
| | | | nucleotidase | | | | | | | |
| sp\|P13674\|P4HA1_HUMAN | 10 | P4HA1 | P4HA1_HUMAN Prolyl | 534 | 0 | 11 | 0 | 0 | 0.00130566 | 0 |
| | | | 4-hydroxylase subunit | | | | | | | |
| | | | alpha-1 | | | | | | | |
| sp\|O15460\|P4HA2_HUMAN | 4 | P4HA2 | P4HA2_HUMAN Prolyl | 535 | 0 | 4 | 0 | 0 | 0.000473898 | 0 |
| | | | 4-hydroxylase subunit | | | | | | | |
| | | | alpha-2 | | | | | | | |
| sp\|P07237\|PDIA1_HUMAN | 4 | P4HB | PDIA1_HUMAN | 508 | 0 | 4 | 0 | 0 | 0.000499085 | 0 |
| | | | Protein disulfide- | | | | | | | |
| | | | isomerase | | | | | | | |
| sp\|Q13310-3\|PABP4_HUMAN | 6 | PABPC4 | PABP4_HUMAN | 660 | 0 | 3 | 3 | 0 | 0.000288108 | 0.000282924 |
| | | | Isoform 3 of | | | | | | | |
| | | | Polyadenylate-binding | | | | | | | |
| | | | protein 4 | | | | | | | |
| sp\|Q8WX93\|PALLD_HUMAN | 1 | PALLD | PALLD_HUMAN | 1383 | 0 | 1 | 0 | 0 | 4.58307E−05 | 0 |
| | | | Palladin | | | | | | | |
| sp\|Q9NVD7\|PARVA_HUMAN | 1 | PARVA | PARVA_HUMAN | 372 | 0 | 1 | 0 | 0 | 0.000170387 | 0 |
| | | | Alpha-parvin | | | | | | | |
| sp\|Q96HS1\|PGAM5_HUMAN | 1 | PGAM5 | PGAM5_HUMAN | 289 | 0 | 0 | 1 | 0 | 0 | 0.000215374 |
| | | | Serine/threonine- | | | | | | | |
| | | | protein phosphatase | | | | | | | |
| | | | PGAM5, mitochondrial | | | | | | | |
| sp\|Q9UHX1\|PUF60_HUMAN | 4 | PUF60 | PUF60_HUMAN | 559 | 0 | 4 | 0 | 0 | 0.000453552 | 0 |
| | | | Poly(U)-binding- | | | | | | | |
| | | | splicing factor PUF60 | | | | | | | |
| sp\|P11234\|RALB_HUMAN | 1 | RALB | RALB_HUMAN Ras- | 206 | 0 | 1 | 0 | 0 | 0.000307688 | 0 |
| | | | related protein Ral-B | | | | | | | |
| sp\|P62826\|RAN_HUMAN | 2 | RAN | RAN_HUMAN GTP- | 216 | 0 | 0 | 2 | 0 | 0 | 0.000576326 |
| | | | binding nuclear | | | | | | | |
| | | | protein Ran | | | | | | | |
| sp\|P54136\|SYRC_HUMAN | 1 | RARS | SYRC_HUMAN | 660 | 0 | 1 | 0 | 0 | 9.60361E−05 | 0 |
| | | | Arginine--tRNA ligase, | | | | | | | |
| | | | cytoplasmic | | | | | | | |
| sp\|P78332\|RBM6_HUMAN | 5 | RBM6 | RBM6_HUMAN RNA- | 1123 | 0 | 0 | 5 | 0 | 0 | 0.000277129 |
| | | | binding protein 6 | | | | | | | |
| sp\|Q6XE24\|RBMS3_HUMAN | 1 | RBMS3 | RBMS3_HUMAN RNA- | 437 | 0 | 1 | 0 | 0 | 0.000145043 | 0 |
| | | | binding motif, single- | | | | | | | |
| | | | stranded-interacting | | | | | | | |
| | | | protein 3 | | | | | | | |
| sp\|P26373\|RL13_HUMAN | 6 | RPL13 | RL13_HUMAN 60S | 211 | 0 | 2 | 4 | 0 | 0.000600795 | 0.00117997 |
| | | | ribosomal protein L13 | | | | | | | |
| sp\|P61353\|RL27_HUMAN | 1 | RPL27 | RL27_HUMAN 60S | 136 | 0 | 1 | 0 | 0 | 0.000466058 | 0 |
| | | | ribosomal protein L27 | | | | | | | |
| sp\|P42766\|RL35_HUMAN | 1 | RPL35 | RL35_HUMAN 60S | 123 | 0 | 0 | 1 | 0 | 0 | 0.000506042 |
| | | | ribosomal protein L35 | | | | | | | |
| sp\|P18124\|RL7_HUMAN | 2 | RPL7 | RL7_HUMAN 60S | 248 | 0 | 2 | 2 | 0 | 0.00051116 | 0.000501961 |
| | | | ribosomal protein L7 | | | | | | | |
| sp\|P05388\|RLA0_HUMAN | 2 | RPLP0 | RLA0_HUMAN 60S | 317 | 0 | 1 | 1 | 0 | 0.000199949 | 0.000196351 |
| | | | acidic ribosomal | | | | | | | |
| | | | protein P0 | | | | | | | |
| sp\|P04843\|RPN1_HUMAN | 1 | RPN1 | RPN1_HUMAN | 607 | 0 | 2 | 0 | 0 | 0.000208843 | 0 |
| | | | Dolichyl- | | | | | | | |
| | | | diphosphooligosaccharide-- | | | | | | | |
| | | | protein | | | | | | | |
| | | | glycosyltransferase | | | | | | | |
| | | | subunit 1 | | | | | | | |
| sp\|P04844\|RPN2_HUMAN | 2 | RPN2 | RPN2_HUMAN | 631 | 0 | 2 | 0 | 0 | 0.0002009 | 0 |
| | | | Dolichyl- | | | | | | | |
| | | | diphosphooligosaccharide-- | | | | | | | |
| | | | protein | | | | | | | |
| | | | glycosyltransferase | | | | | | | |
| | | | subunit 2 | | | | | | | |
| sp\|P62280\|RS11_HUMAN | 4 | RPS11 | RS11_HUMAN 40S | 158 | 0 | 2 | 2 | 0 | 0.000802327 | 0.000787889 |
| | | | ribosomal protein S11 | | | | | | | |
| sp\|P62263\|RS14_HUMAN | 1 | RPS14 | RS14_HUMAN 40S | 151 | 0 | 2 | 0 | 0 | 0.000839521 | 0 |
| | | | ribosomal protein S14 | | | | | | | |
| sp\|P62244\|RS15A_HUMAN | 2 | RPS15A | RS15A_HUMAN 40S | 130 | 0 | 1 | 1 | 0 | 0.000487568 | 0.000478794 |
| | | | ribosomal protein S15a | | | | | | | |
| sp\|P62249\|RS16_HUMAN | 1 | RPS16 | RS16_HUMAN 40S | 146 | 0 | 1 | 0 | 0 | 0.000434136 | 0 |
| | | | ribosomal protein S16 | | | | | | | |
| sp\|P42677\|RS27_HUMAN | 2 | RPS27 | RS27_HUMAN 40S | 84 | 0 | 1 | 1 | 0 | 0.000754569 | 0.00074099 |
| | | | ribosomal protein S27 | | | | | | | |
| sp\|P23396-2\|RS3_HUMAN | 6 | RPS3 | RS3_HUMAN Isoform | 259 | 0 | 3 | 3 | 0 | 0.000734176 | 0.000720964 |
| | | | 2 of 40S ribosomal | | | | | | | |
| | | | protein S3 | | | | | | | |
| sp\|P62241\|RS8_HUMAN | 5 | RPS8 | RS8_HUMAN 40S | 208 | 0 | 2 | 3 | 0 | 0.00060946 | 0.000897738 |
| | | | ribosomal protein S8 | | | | | | | |
| sp\|P46781\|RS9_HUMAN | 2 | RPS9 | RS9_HUMAN 40S | 194 | 0 | 0 | 2 | 0 | 0 | 0.000641682 |
| | | | ribosomal protein S9 | | | | | | | |
| sp\|Q9P2E9\|RRBP1_HUMAN | 6 | RRBP1 | RRBP1_HUMAN | 1410 | 0 | 6 | 0 | 0 | 0.000269718 | 0 |
| | | | Ribosome-binding | | | | | | | |
| | | | protein 1 | | | | | | | |
| sp\|Q15424-3\|SAFB1_HUMAN | 3 | SAFB | SAFB1_HUMAN | 917 | 0 | 0 | 3 | 0 | 0 | 0.000203631 |
| | | | Isoform 3 of Scaffold | | | | | | | |
| | | | attachment factor B1 | | | | | | | |
| sp\|P50454\|SERPH_HUMAN | 10 | SERPINH1 | SERPH_HUMAN Serpin | 418 | 0 | 10 | 0 | 0 | 0.00151636 | 0 |
| | | | H1 | | | | | | | |
| sp\|Q7Z333-4\|SETX_HUMAN | 1 | SETX | SETX_HUMAN Isoform | 2706 | 0 | 0 | 1 | 0 | 0 | 2.30019E−05 |
| | | | 4 of Probable helicase | | | | | | | |
| | | | senataxin | | | | | | | |
| sp\|Q92629-2\|SGCD_HUMAN | 1 | SGCD | SGCD_HUMAN | 290 | 0 | 1 | 0 | 0 | 0.000218565 | 0 |
| | | | Isoform 2 of Delta- | | | | | | | |
| | | | sarcoglycan | | | | | | | |
| sp\|O60264\|SMCA5_HUMAN | 0 | SMARCA5 | SMCA5_HUMAN | 1052 | 0 | 0 | 4 | 0 | 0 | 0.000236666 |
| | | | SWI/SNF-related | | | | | | | |
| | | | matrix-associated | | | | | | | |
| | | | actin-dependent | | | | | | | |
| | | | regulator of chromatin | | | | | | | |
| | | | subfamily A member 5 | | | | | | | |
| sp\|Q9Y651\|SOX21_HUMAN | 0 | SOX21 | SOX21_HUMAN | 276 | 0 | 0 | 1 | 0 | 0 | 0.000225519 |
| | | | Transcription factor | | | | | | | |
| | | | SOX-21 | | | | | | | |
| sp\|Q13813-2\|SPTN1_HUMAN | 4 | SPTAN1 | SPTN1_HUMAN | | | | | | 7.67668E−05 | 2.51285E−05 |
| | | | Isoform 2 of Spectrin | | | | | | | |
| | | | alpha chain, non- | 2477 | 0 | 3 | 1 | 0 | | |
| | | | erythrocytic 1 | | | | | | | |
| sp\|Q01082\|SPTB2_HUMAN | 4 | SPTBN1 | SPTB2_HUMAN | 2364 | 0 | 5 | 0 | 0 | 0.000134061 | 0 |
| | | | Spectrin beta chain, | | | | | | | |
| | | | non-erythrocytic 1 | | | | | | | |
| sp\|Q9UQ35\|SRRM2_HUMAN | 0 | SRRM2 | SRRM2_HUMAN | 2752 | 0 | 0 | 3 | 0 | 0 | 6.78523E−05 |
| | | | Serine/arginine | | | | | | | |
| | | | repetitive matrix | | | | | | | |
| | | | protein 2 | | | | | | | |
| sp\|O75494\|SRS10_HUMAN | 1 | SRSF10 | SRS10_HUMAN | 262 | 0 | 1 | 0 | 0 | 0.000241923 | 0 |
| | | | Serine/arginine-rich | | | | | | | |
| | | | splicing factor 10 | | | | | | | |
| sp\|Q04837\|SSBP_HUMAN | 1 | SSBP1 | SSBP_HUMAN Single- | 148 | 0 | 0 | 1 | 0 | 0 | 0.000420562 |
| | | | stranded DNA-binding | | | | | | | |
| | | | protein, mitochondrial | | | | | | | |
| sp\|O60506\|HNRPQ_HUMAN | 1 | SYNCRIP | HNRPQ_HUMAN | 623 | 0 | 1 | 0 | 0 | 0.00010174 | 0 |
| | | | Heterogeneous | | | | | | | |
| | | | nuclear | | | | | | | |
| | | | ribonucleoprotein Q | | | | | | | |
| sp\|P21980\|TGM2_HUMAN | 2 | TGM2 | TGM2_HUMAN | 687 | 0 | 2 | 0 | 0 | 0.000184523 | 0 |
| | | | Protein-glutamine | | | | | | | |
| | | | gamma- | | | | | | | |
| | | | glutamyltransferase 2 | | | | | | | |
| sp\|P07996\|TSP1_HUMAN | 1 | THBS1 | TSP1_HUMAN | 1170 | 0 | 4 | 0 | 0 | 0.000216697 | 0 |
| | | | Thrombospondin-1 | | | | | | | |
| sp\|Q9UPQ9\|TNR6B_HUMAN | 3 | TNRC6B | TNR6B_HUMAN | 1833 | 0 | 2 | 1 | 0 | 6.91586E−05 | 0.000033957 |
| | | | Trinucleotide repeat- | | | | | | | |
| | | | containing gene 6B | | | | | | | |
| | | | protein | | | | | | | |
| sp\|P62995\|TRA2B_HUMAN | 1 | TRA2B | TRA2B_HUMAN | 288 | 0 | 1 | 0 | 0 | 0.000220083 | 0 |
| | | | Transformer-2 protein | | | | | | | |
| | | | homolog beta | | | | | | | |
| sp\|Q14157-5\|UBP2L_HUMAN | 2 | UBAP2L | UBP2L_HUMAN | 1104 | 0 | 1 | 1 | 0 | 5.74129E−05 | 5.63797E−05 |
| | | | Isoform 5 of Ubiquitin- | | | | | | | |
| | | | associated protein 2- | | | | | | | |
| | | | like | | | | | | | |
| sp\|P0CG48\|UBC_HUMAN | 2 | UBC | UBC_HUMAN | 685 | 0 | 1 | 1 | 0 | 9.25311E−05 | 0.000090866 |
| | | | Polyubiquitin-C | | | | | | | |
| sp\|P22695\|QCR2_UMAN | 1 | UQCRC2 | QCR2_HUMAN | 453 | 0 | 1 | 0 | 0 | 0.00013992 | 0 |
| | | | Cytochrome b-c1 | | | | | | | |
| | | | complex subunit 2, | | | | | | | |
| | | | mitochondrial | | | | | | | |
| sp\|P0C7P4\|UCRIL_HUMAN | 1 | UQCRFS1P1 | UCRIL HUMAN | 283 | 0 | 1 | 0 | 0 | 0.000223971 | 0 |
| | | | Putative cytochrome | | | | | | | |
| | | | b-c1 complex subunit | | | | | | | |
| | | | Rieske-like protein 1 | | | | | | | |
| sp\|Q14146\|URB2_HUMAN | 0 | URB2 | URB2_HUMAN | 1524 | 0 | 0 | 1 | 0 | 0 | 0.000040842 |
| | | | Unhealthy ribosome | | | | | | | |
| | | | biogenesis protein 2 | | | | | | | |
| | | | homolog | | | | | | | |
| sp\|P08670\|VIME_HUMAN | 1 | VIM | VIME_HUMAN | 466 | 0 | 1 | 0 | 0 | 0.000136017 | 0 |
| | | | Vimentin | | | | | | | |
| sp\|Q9H0D6\|XRN2_HUMAN | 1 | XRN2 | XRN2_HUMAN 5′-3′ | 950 | 0 | 0 | 1 | 0 | 0 | 6.55192E−05 |
| | | | exoribonuclease 2 | | | | | | | |
| sp\|P49750-4\|YLPM1_HUMAN | 13 | YLPM1 | YLPM1_HUMAN | 2146 | 0 | 9 | 4 | 0 | 0.000265822 | 0.000116017 |
| | | | Isoform 4 of YLP motif- | | | | | | | |
| | | | containing protein 1 | | | | | | | |
| sp\|O75152\|ZC11A_HUMAN | 0 | ZC3H11A | ZC11A_HUMAN Zinc | 810 | 0 | 0 | 1 | 0 | 0 | 7.68435E−05 |
| | | | finger CCCH domain- | | | | | | | |
| | | | containing protein 11A | | | | | | | |
| sp\|Q5BKZ1\|ZN326_HUMAN | 3 | ZNF326 | ZN326_HUMAN DBIRD | 582 | 0 | 0 | 7 | 0 | 0 | 0.00074863 |
| | | | complex subunit | | | | | | | |
| | | | ZNF326 | | | | | | | |

TABLE 7C
Aska-SS −+ shSSX

| | Aska | Aska | |
|---|---|---|---|
| | (shControl)/Aska | (shControl)/Aska | |
| | (shSS18-SSX) | (shSS18-SSX) | |
| | LFQ Intensity | LFQ Intensity | |
| | (Norm to | (Norm to | |
| | SMARCA4 | SMARCA4 | % |
| Gene names | Rep 1) | Rep 2) | Coverage |
| SSX1; SSX8 | 8.69195075 | 66.78165769 | 12.2 |
| SMARCA4 | 1 | 1 | 43 |
| SS18 | 0.959463613 | 1.263985576 | 7.7 |
| BCL7C | 1.744253828 | 1.537120215 | 57.1 |
| BCL7B | 1.339078001 | 1.126236646 | 21.8 |
| ACTL6A | 0.783942638 | 1.104979803 | 56.6 |
| SMARCE1 | 0.893791358 | 0.854705908 | 61.8 |
| SMARCD1 | 0.770993178 | 0.949816024 | 79.4 |
| SMARCC1 | 0.505600572 | 0.622079125 | 47 |
| ARID1B | 0.721229344 | 0.649778539 | 41 |
| SMARCB1 | 0.022092646 | 0.063656869 | 60.9 |

Finally, to evaluate whether SS18-SSX-containing BAF complexes that are tethered to the nucleosome acidic patch via SSX in place of the BAF core module SMARCB1 C-terminal acidic patch binding region (Valencia et al. (2019) Cell 179:1342-1356) are competent in remodeling, chromatin remodeling assays were performed using restriction enzyme accessibility assays (REAA) on endogenous BAF complexes containing either SS18 WT or SS18-SSX as well as assay for transposase-accessible chromatin using sequencing (ATAC-seq) in both CRL7250 fibroblasts and SS cell lines. Remodeling efficiency and ATPase activity of SS18-SSX-bound BAF complexes was slightly lower than that of WT SS18-bound complexes (FIGS. 7I, 10E and 10G), however, this reduced activity was sufficient to enable DNA accessibility over SS18-SSX target sites genome-wide (FIGS. 7J and 10F).

Taken together, these data resolve SSX as a nucleosome acidic patch binding ligand fused to SS18, a subunit bound to the BAF complex ATPase subunit, SMARCA4 at its N-terminal region within the core structural module (Valencia et al. (2019) Cell 179:1342-1356; Ye et al. (2019) Science doi:10.1126/science.aay0033), that dominantly competes for acidic patch binding with BAF core module subunit SMARCB1, resulting in its partial destabilization and degradation. These oncogenic SS18-SSX-containing complexes are still proficient in chromatin remodeling and catalytic activity, resulting in the aberrant activation of normally repressed chromatin regions.

### Example 5: SSX Exhibits Preference for H2A K119Ub-Marked Nucleosomes, Mediated by its Conserved C-Terminal Acidic Region

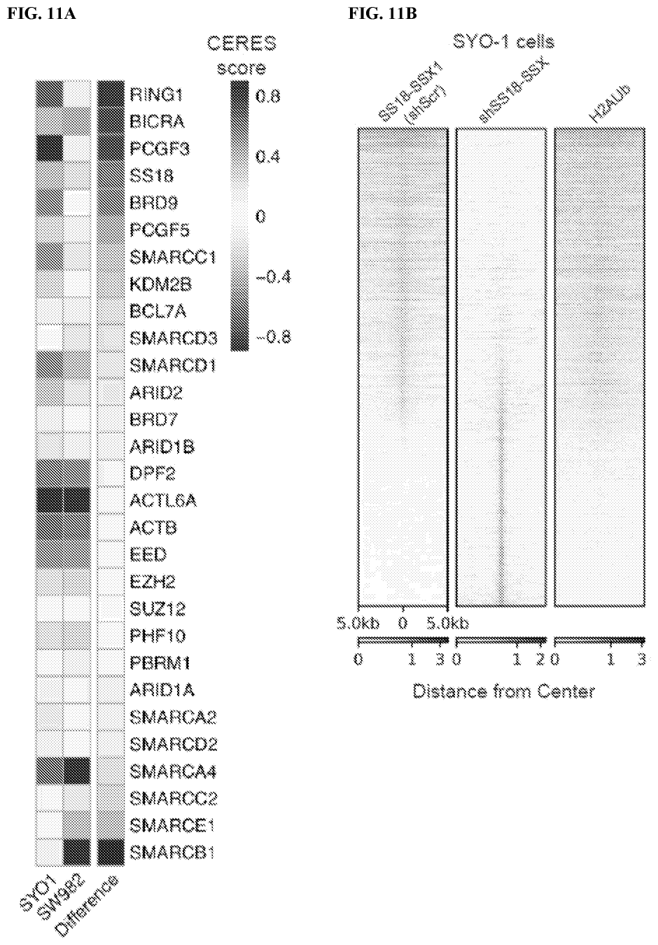

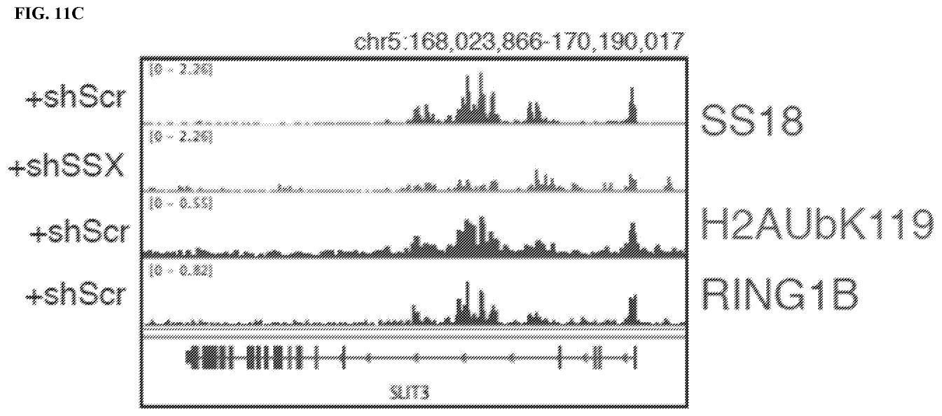

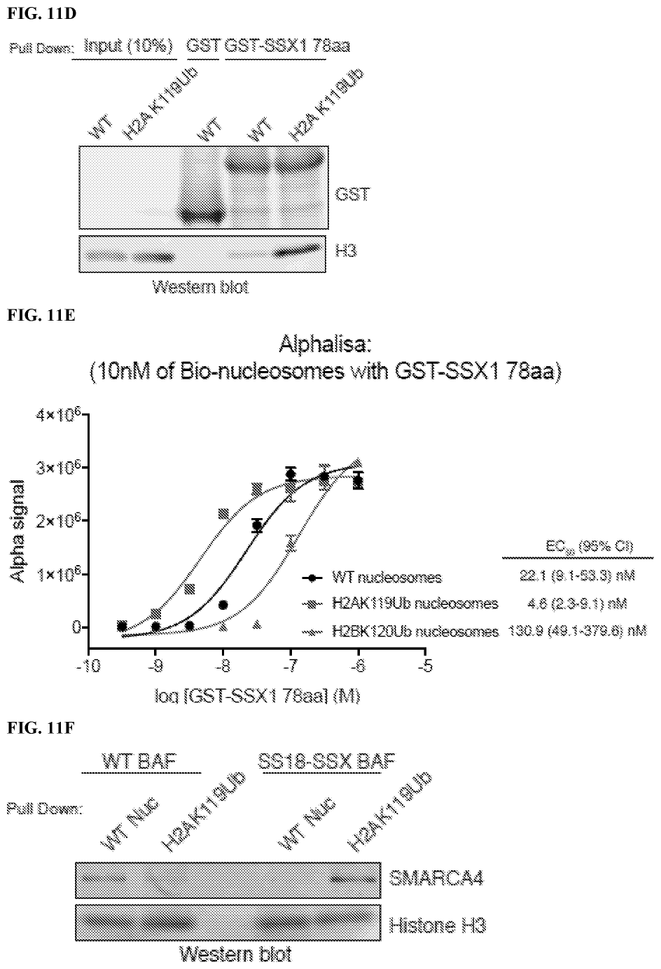

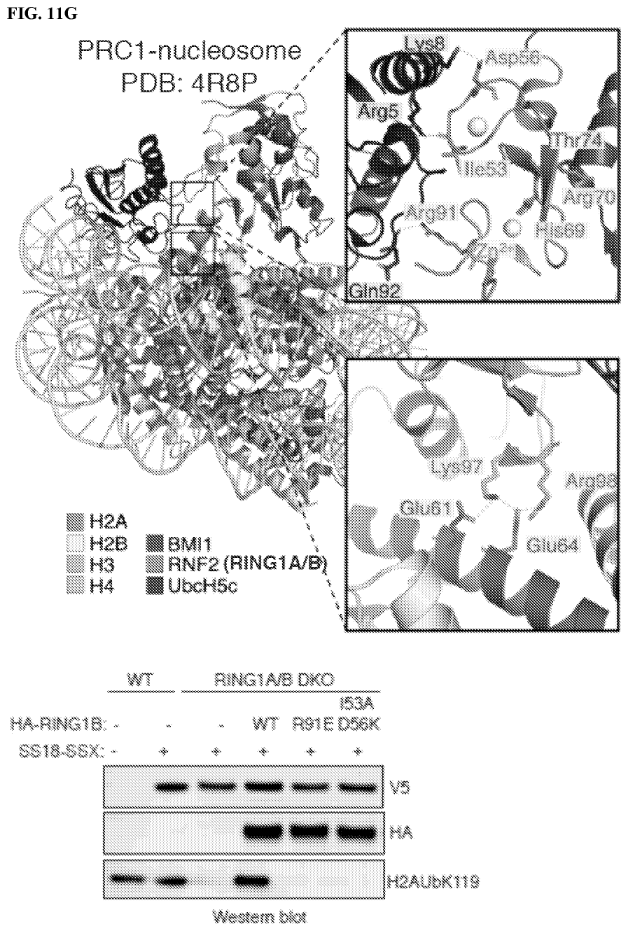

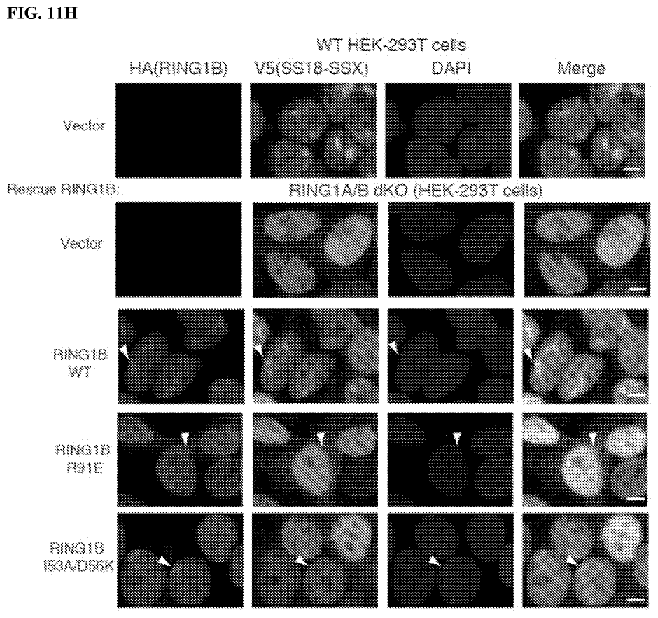

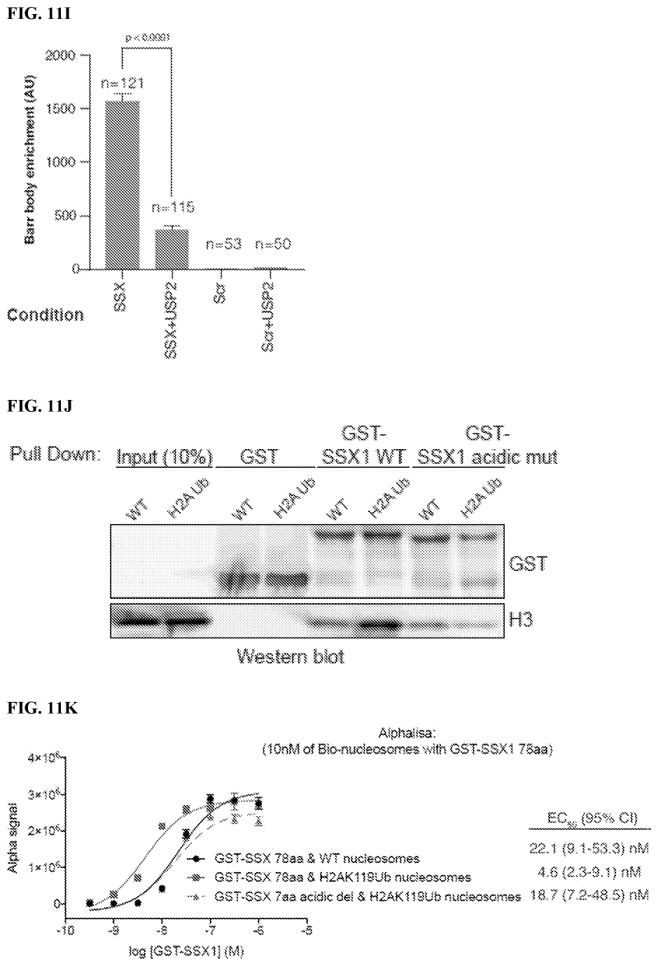

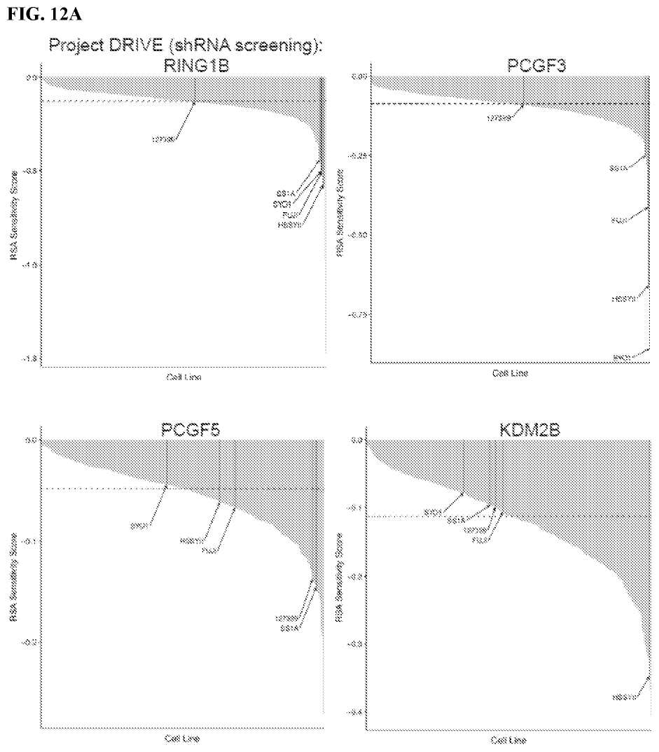

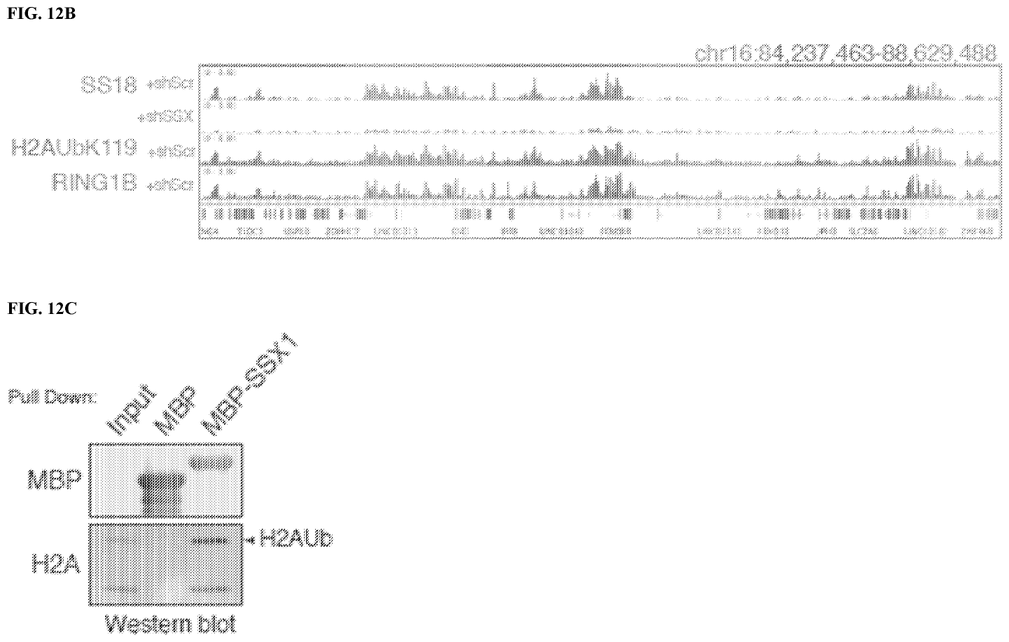

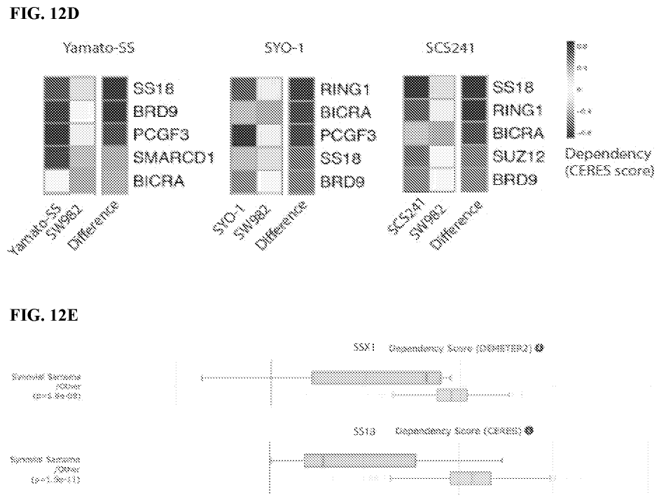

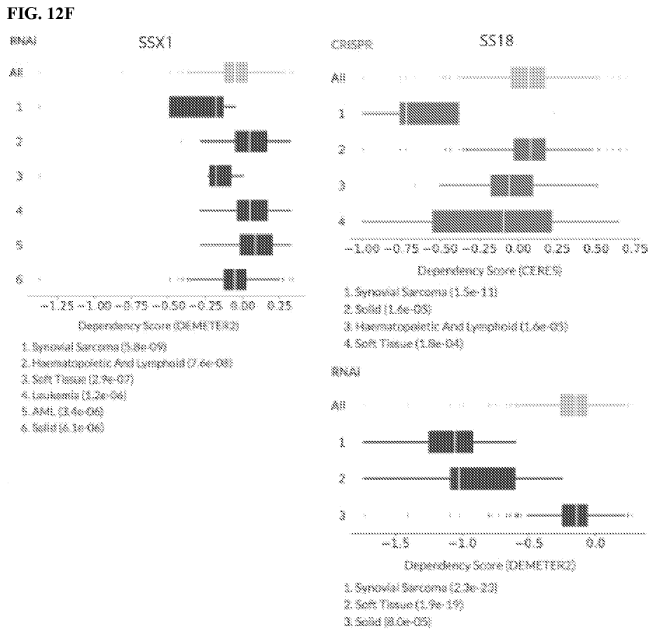

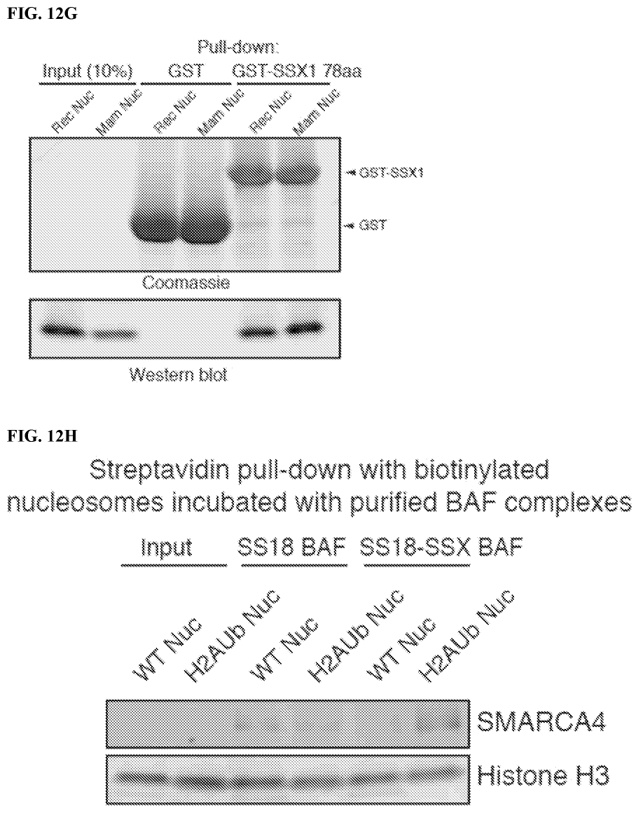

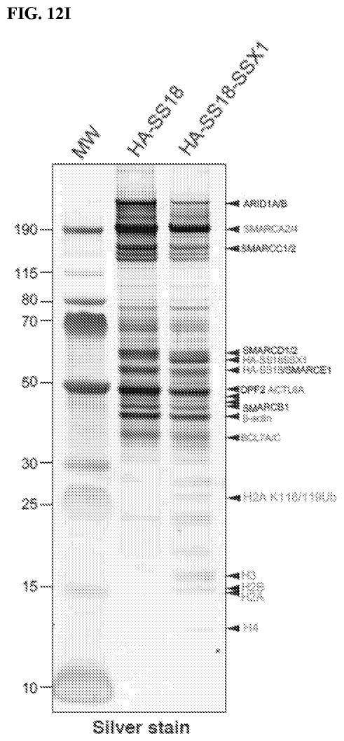

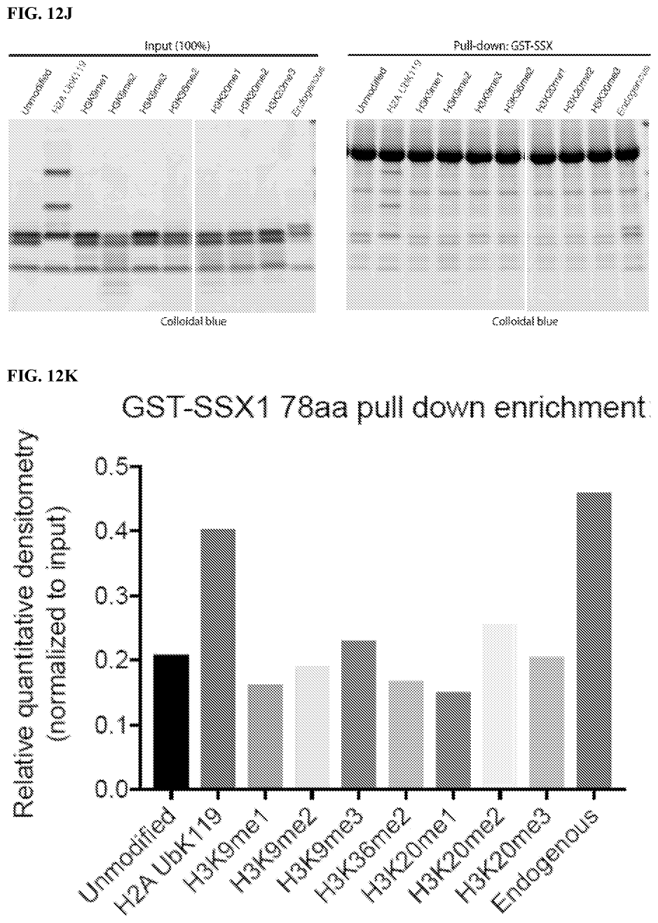

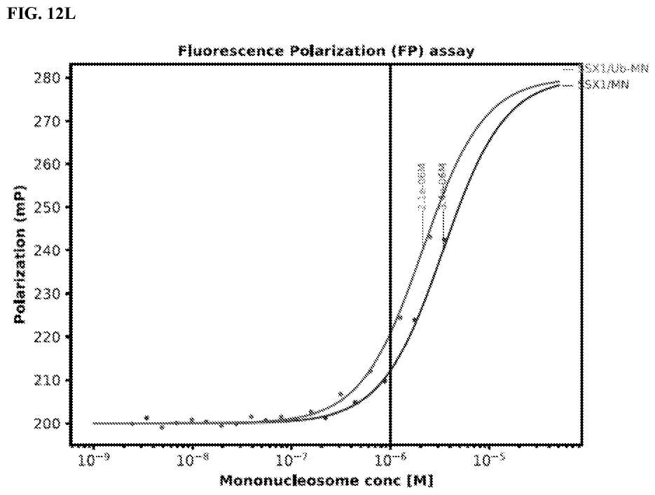

Previously, it was found that SS18-SSX-bound BAF complexes localize to polycomb-repressed regions (McBride et al. (2018) Cancer Cell 33:1128-1141). The engagement between the conserved SSX basic region and the nucleosome acidic patch is not, in itself, sufficient to explain why SS18-SSX complexes are preferentially recruited to repressed chromatin. It was therefore reasoned that the SSX-nucleosome acidic patch interaction can be augmented in some manner by the presence of specific histone repressive marks. To explore this possibility, CRISPR-Cas9-based screening of genes encoding proteins that are responsible for decorating and maintaining repressive chromatin was performed. These studies were performed in the SS cell line, SYO-1, as well as in a cell line that is an SS histologic mimic lacking the SS18-SSX fusion, SW982 (FIG. 11A). Notably, it was found that PRC1 subunits (specifically, Ring1A/B, as well as PCGF5 and PCGF3 components of PRC1.3 and PRC1.5 complexes) were selectively enriched as synthetic lethal dependencies in SS cell lines SYO1 as well as other SS cell lines including Yamato and SCS241 (FIGS. 11A, 12A and 12D). Importantly, all SS cell lines profiled exhibited significant dependency on SS18 and SSX (and hence the SS18-SSX fusion), relative to all other cell lines profiled (FIGS. 12E and 12F).

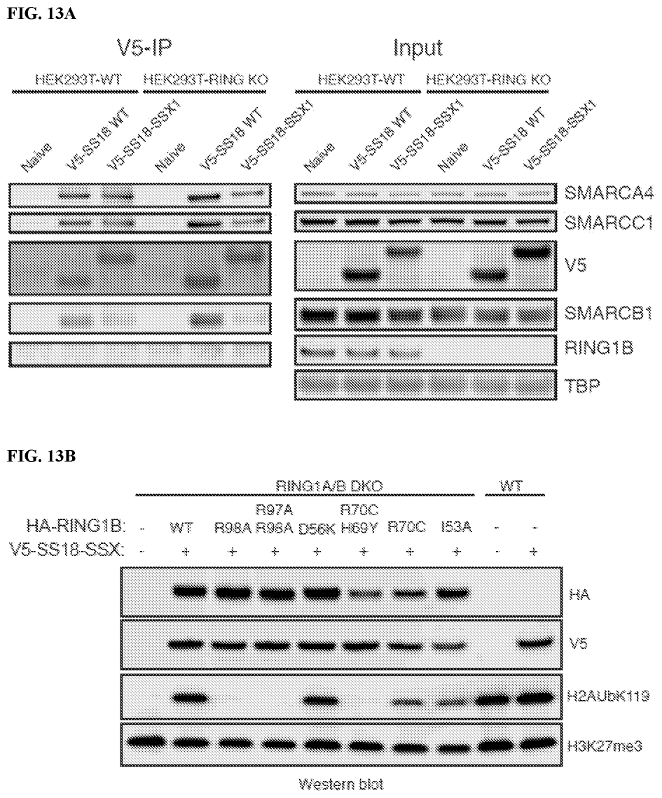

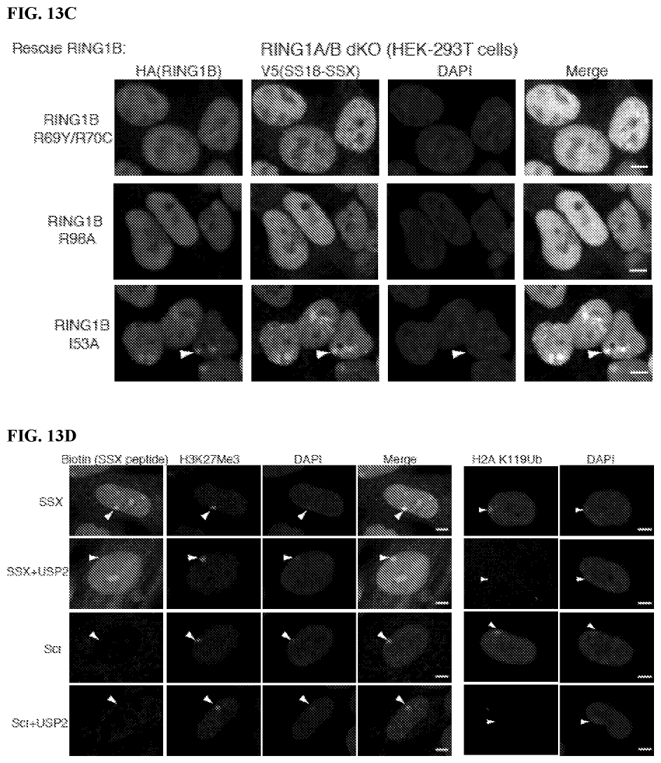

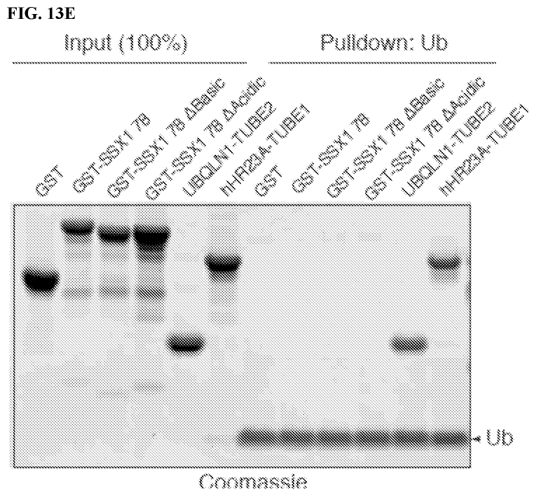

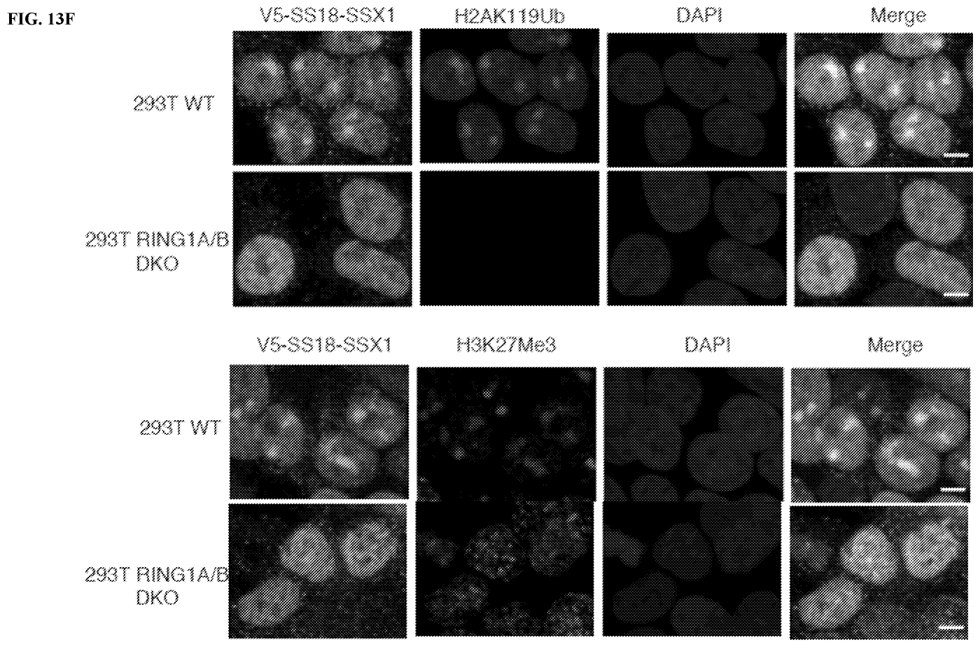

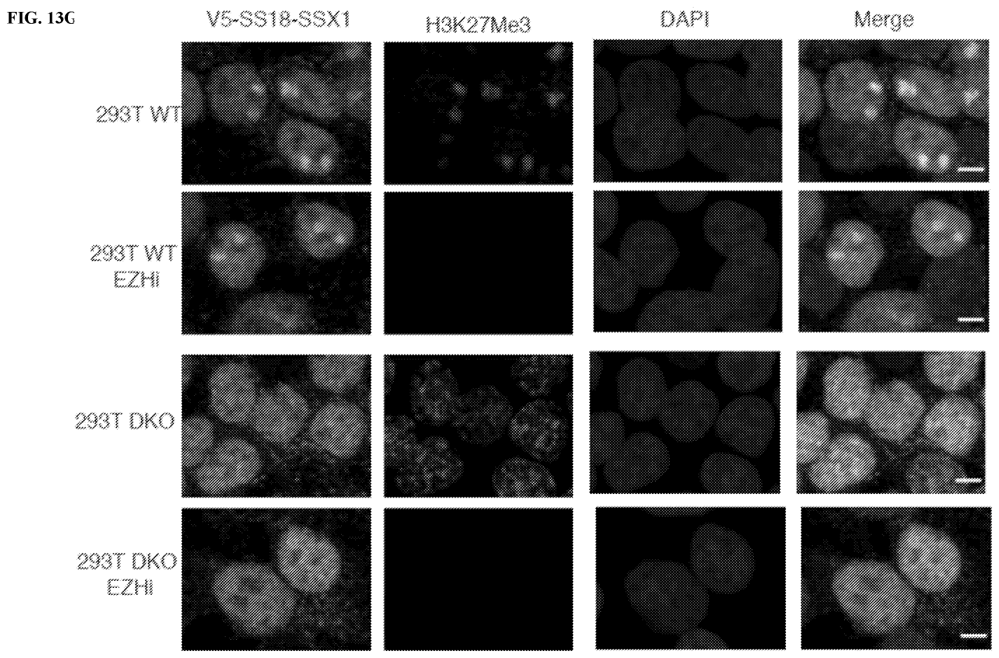

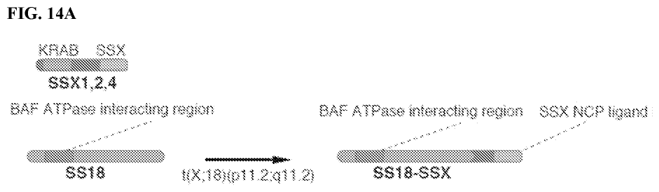

Given that the key histone modification placed by PRC1 is the H2A K119Ub mark, it was determined whether SSX exhibited any preferential binding to nucleosomes decorated with this modification. Notably, it was found that in SS cell lines, H2AK119Ub directly co-localized with sites of SS18-SSX BAF complex occupancy (FIGS. 11B-11C and FIG. 12B). This was consistent with the IF observations indicating substantial co-localization at Barr bodies (FIGS. 3C, 5A, 5B, 5D, 6C and 9C). Indeed, pulldown experiments and AlphaLisa binding assays performed with GST-SSX 78aa protein revealed significantly higher affinity to H2A K119Ub-decorated nucleosomes relative to unmodified nucleosomes or H2B K120Ub nucleosomes (FIGS. 11D-11E and 12G). Incubation of SSX 78aa with mammalian mononucleosomes also captured the higher molecular weight H2AUb species (FIG. 12C), as did SS18-SSX-bound BAF complexes (FIGS. 1A-1B, Tables 5A-5E). Importantly, endogenously purified SS18-SSX-bound BAF complexes enriched for binding of recombinant H2A K119Ub-modified nucleosomes over unmodified nucleosomes (FIGS. 11F, 12H and 12I), consistent with the finding that SS18-SSX fusion target sites directly overlay H2AK119Ub sites genome-wide in SS cell lines (FIG. 11B). Finally, a screen for SSX binding to a range of differentially-marked recombinant mononucleosomes as well as mammalian (pooled) nucleosomes was performed, and again, it was identified that GST-SSX 78aa preferentially bound to H2A K119Ub and mammalian nucleosomes over unmodified nucleosomes or nucleosomes with other histone marks (FIGS. 12J-K). Fluorescence polarization (FP) experiments performed using fluoro-labeled SSX (in place of GST tag) also confirmed higher binding affinity to H2A K119Ub-decorated nucleosomes compared to unmodified nucleosomes (FIG. 12I). To understand the role of H2A K119Ub in SSX-BAF localization, the core, catalytic subunits of the PRC1 complex, RING1A and RING1B, were next double deleted using CRISPR-Cas9 in HEK-293T cells (RING1A/1B-dKO HEK-293T cells) and expressed SS18-SSX (FIG. 13A). Following immunofluorescence, complete loss of SS18-SSX localization to Barr bodies was observed as compared to RING1A/B WT cells (FIGS. 11G-11H). To address whether the catalytic activity of PRC1 rather than PRC1 complex formation is required for SS18-SSX Barr body recruitment, structure-guided mutagenesis was performed to selectively disrupt the ubiquitin ligase activity of PRC1 and hence block its placement of H2A K119Ub (FIG. 11G). a series of point mutations in RING1B were designed to disrupt acidic patch recognition (R98A), zinc binding (H69Y/R70C) and the E2 binding interface (R91A and I53A/D56K (Blackledge et al. (2019) BioRxiv 667667, doi:10.1101/667667)) (FIGS. 11G-11H and 13B-13C). Rescue of WT RING1B in RING1A/1B-dKO cells was able to completely rescue SSX localization. However, restoration of RING1B mutant variants affected SS18-SSX localization in a manner directly proportional to the degree to which these RING1B mutations impacted H2A K119Ub deposition. Significantly for this study, RING1A ligase-deficient R91E and I53A/D56K were able to form polycomb foci but were unable to recruit SS18-SSX, further highlighting the importance of the H2A K119Ub mark placement. As controls, R98A, and combined H69Y/R70C mutants had similar loss-of-function effects on SS18-SSX localization (Wang et al. (2004) Nature 431:873-878; McGinty et al. (2014) Nature 514:591-596) (FIGS. 11G-11H and 13B-13C). The widely-used I53A mutant (Buchwald et al. (2006) EMBO J. 25:2465-2474; Eskeland et al. (2010) Mol. Cell 38:452-464; Illingworth et al. (2015) Genes & Dev. 29:1897-1902) only partially attenuated H2A ubiquitination, and therefore had little effect on SSX targeting. As further support for a role for H2A K119Ub in SSX recruitment, a peptide hybridization assay performed on IMR90 cells pretreated with the deubiquitinating enzyme, USP2 was used. USP2-mediated removal of H2A K119Ub disrupted SSX peptide hybridization to Barr bodies specifically and without affecting its overall nuclear staining pattern, consistent with the general ability of SSX to bind unmodified nucleosomes via its acidic patch binding region (FIGS. 11I and 13D). EZH2 inhibitor treatment performed in WT HEK-293T or RING1A/B dKO HEK-293T cells further highlighted the requirement for H2A Ub119 placement (and hence PRC1) rather than H3K27me3 and PRC2 activity (FIGS. 13F-13G). Somewhat surprisingly, given the clear role for H2A K119Ub in recruiting SSX to chromatin, direct binding between SSX and free ubiquitin was not observed, as assessed by a Ub-agarose pull down assay (FIG. 13E), however, it is conceivable that SSX only engages Ub in the context of H2AK119Ub nucleosomes, as seen with other readers such as Dot1L (Anderson et al. (2019) Cell Rep. 26:1681-1690; Worden et al. (2019) Cell 176:1490-1501; Valencia-Sanchez et al. (2019) Mol. Cell 74:1010-1019), or alternatively, that it can recognize specific features of the nucleosome core itself that are sterically or allosterically affected by the presence of the ubiquitylation mark.

Finally, given that the conserved C-terminal acidic region of SSX did not disrupt SSX-nucleosome binding (FIG. 3F) but did affect SS18-SSX-specific BAF complex targeting and resultant gene expression and proliferation (FIGS. 3G-3I) in a manner comparable to loss of the basic region (acidic patch binding region), it was determined whether this region mediates the preference of SSX for H2A K119Ub-decorated nucleosomes. Excitingly, it was found that mutation of the C-terminal acidic region of SSX to alanines (i.e., DPEEDDE (SEQ ID NO: 221) to AAAAAAA (SEQ ID NO: 222)) relieved the preference of SSX for H2AK119Ub nucleosomes, while not altering SSX binding to nucleosomes (FIGS. 11J-11K). These data collectively indicate that the conserved C-terminal acidic amino acids are required to drive the preference of SSX for H2A K119Ub nucleosomes and hence SS18-SSX-bound BAF complex targeting to repressive regions genome-wide, as observed in cells.

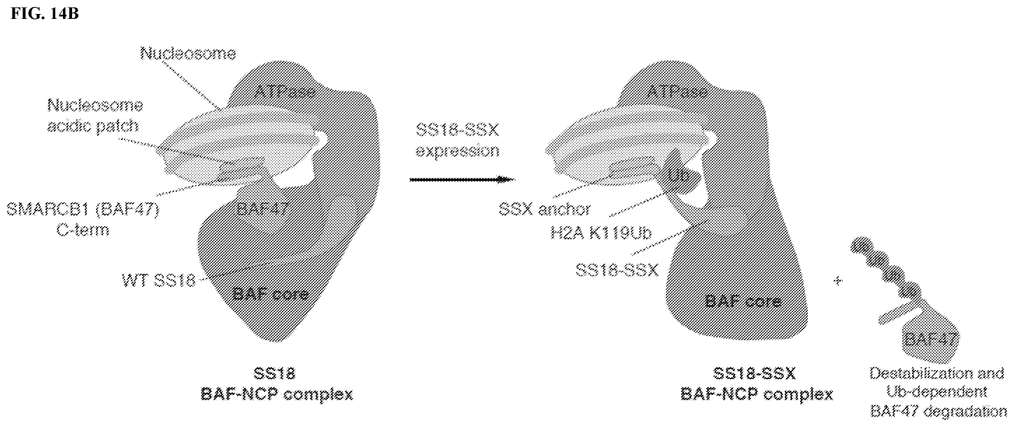

Here an unexpected set of properties have been identified and their functional ramifications were found in the fusion oncoprotein, SS18-SSX, the oncogenic driver of human synovial sarcoma (FIGS. 14A-14B, model). An unusual, and new reported case in which an additional nucleosome acidic patch binding domain is fused to a subunit of a major chromatin remodeling complex, the mammalian SWI/SNF (BAF) complex, causing a generally tumor suppressor complex to gain oncogenic properties was found. Although several SNF2 helicase-based chromatin remodeling complexes are increasingly recognized to require the H2A/H2B nucleosome binding hub, it was found that the minimal, conserved SSX 34 aa region dominantly binds the acidic patch of nucleosomes, with preference for H2A K119Ub histone modification, altering the interaction between the nucleosome-SMARCB1 C-terminal alpha helix- interaction found in WT BAF complexes (Valencia et al. (2019) Cell 179:1342-1356; Ye et al. (2019) Science, doi:10.1126/science.aay0033), and resulting in higher affinity nucleosome binding properties augmented by specific repressive histone mark preferences (Valencia et al. (2019) Cell 179:1342-1356). While these data coupled with recent structural insights in yeast RSC and SWI/SNF complexes provide strong support for SS18-SSX-mediated displacement of SMARCB1 from the acidic patch and its destabilization at the nucleosome-proximal region of the core (base) module of BAF complexes, a high resolution, 3D structure of human BAF complexes containing SS18, as well as those containing SS18-SSX will be needed to define the full repertoire of structural changes to nucleosome-bound BAF complexes upon incorporation of SS18-SSX. This is particularly true given that the SS18 subunit is metazoan-specific and hence is not found in yeast complexes.

The expression of full length SSX is normally restricted to testes where it likely plays a role in sperm development, potentially involving polycomb-driven XY-body repression through engagement of H2A K119Ub-decorated sex chromosomes (Baarends et al. (1999) Dev. Biol. 207:322-333). Remarkably, this normal function of SSX as a binder of the nucleosome acidic patch and “reader” of this repressive state is leveraged in synovial sarcoma to alter BAF chromatin remodeling complex localization and gene expression patterns. Normally in testes, full-length SSX can function as a ligand for nucleosomes in this H2A K119Ub repressive state to promote further transcriptional repression through use of its N-terminal KRAB domain (Huntley et al. (2006) Genome Res. 16:669-677). In the case of SS, the KRAB domain is lost and replaced with essentially the whole ATPase module of the BAF chromatin remodeling complex via its fusion to SS18. This unfortunate scenario leads to gain of altered repressive chromatin reading properties of BAF complexes, loss of normal BAF complex-nucleosome acidic patch engagement, tight affinity and longer residency times at normally polycomb- repressed regions, and the activation of genes found in these regions (FIGS. 14A-14B, model).

The SSX 78 aa tail, particularly the conserved 34aa C-terminus was therefore characterized (FIG. 3D) as a ligand of the nucleosome acidic patch and the H2A K119Ub histone mark. These data indicate two non-mutually exclusive explanations for this reading preference: H2A K119Ub modification influences nucleosome structure by further exposes the acidic patch binding site; or, SSX exhibits a direct physical engagement with ubiquitin in the nucleosomal context. While studies that indicate that SSX does not bind directly to free (bead-bound) uniquitin was performed (FIG. 13E), this does not rule out the possibility of direct ubiquitin engagement by the acidic C-terminal region of SSX when SS18-SSX-bound complexes are docked on nucleosomes. In this manner, Dot1L, for example, does not bind free ubiquitin but is only poised to interact with H2B UbK120 during substrate engagement (Anderson et al. (2019) Cell Rep. 26:1681-1690; Worden et al. (2019) Cell 176:1490-1501; Valencia-Sanchez et al. (2019) Mol. Cell 74:1010-1019). Understanding this binding preference requires future 3D high resolution structural characterization of SS18-SSX-bound human BAF complexes. Nonetheless, these results indicate that SSX acts as a nucleosome-specific binding ligand for the acidic patch on H2A K119Ub-decorated nucleosomes and that this property underlies the chromatin localization, gene expression, and synthetic lethal dependency profiles of this tumor type.

The biochemical and structural properties of fusion partner SSX elucidated here underpin the dependency of SS on PRC1 complex activity that have been detected in fitness screening efforts and in the structure-guided mutagenesis studies. In contrast to other reports (Banito et al. (2018) Cancer cell 33:527-541), direct binding to PRC1 by the SS18-SSX fusion is not found (or by SSX specifically), as has been indicated, nor a selective dependency on KDM2B; rather, it was found that SS18-SSX-bound complexes bind preferentially to H2A K119Ub-marked nucleosomes, and hence require PRC1 complex activity to place the H2AUbK119 mark. In all MS experiments here, peptides corresponding to PRC1 or PRC2 were not detected, rather, highly abundant peptides corresponding to histones and ubiquitin itself were found, and enrichment of peptides corresponding to the ATPase module subunits of BAF complexes (SMARCA4, BCL7A, beta-actin, ACTL6A) to which SS18 was tethered. The increased abundance of BAF complexes bound to SS18-SSX over PRC1-decorated sites and hence the frequency of molecules co-localized on chromatin can help reconcile these previous indications.

Finally, these results indicate that strategies to directly and specifically inhibit the SSX- or SS18-SSX-bound BAF complex- H2A K119Ub nucleosome interactions can represent viable new strategies for small molecule or inhibitory peptide identification and therapeutic development for synovial sarcoma. In conclusion, this disclosure presents an unexpected nucleosome acidic patch binding function of SSX, a partner within a fusion oncoprotein that lacks a canonical TF DNA-binding domain or recognizable chromatin reader domain and hence has remained a longstanding challenge to understand and target, that drives the altered behavior of the BAF chromatin remodeling complex, activating oncogenic programs in a cancer-specific manner.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

1. A method of treating a subject afflicted with synovial sarcoma comprising administering to the subject a therapeutically effective amount of an agent that inhibits binding of a SS18-SSX fusion protein to a nucleosome, optionally wherein the nucleosome is an H2A K119Ub-marked nucleosome, optionally wherein
a) the SS18-SSX fusion protein comprises a C-terminal region containing a basic region, and an acidic region of a SSX protein, further optionally wherein the basic region comprises a minimal 34-amino acid region;
b) the SS18-SSX fusion protein is selected from Table 2;
c) the SS18-SSX fusion protein comprises SS18 protein fused with a c-terminal portion of a SSX protein;
d) the SS18-SSX fusion protein comprises c-terminal 34 amino acids (aa155-188) of a SSX protein;
e) the SS18-SSX fusion protein comprises c-terminal 78 amino acids (aa 111-188) of a SSX protein;
f) the SSX protein is selected form the group consisting of human SSX1, SSX2, SSX3, SSX4, SSX6, SSX7, SSX8, and SSX9;
g) the SS18-SSX fusion protein comprises W164, R167, L168, R169 and/or R171 of SEQ ID: 3, 7, 13, 17, 21, 25, or 31, or orthologs thereof;
h) the SS18-SSX fusion protein is a part of a BAF complex;
i) the nucleosome comprises H2A protein comprising E56, E64, D90, E91, E92 and/or E113 of human, mouse, rat, or Xenopus H2A, or orthologs thereof; and/or H2B protein comprising E105 and/or E113 of human, mouse, rat, or Xenopus H2B, or orthologs thereof;
j) the subject is an animal model of the cancer, optionally wherein the animal model is a mouse model; and/or
k) the subject is a mammal, optionally wherein the mammal is a mouse or a human.

2-3. (canceled)

4. The method of claim 1, wherein the agent
a) inhibits binding of the basic region of the SS18-SSX fusion protein to an acidic patch of the nucleosome, optionally wherein the nucleosome is an H2A K119Ub-marked nucleosome;
b) is a small molecule inhibitor, a small molecule degrader, CRISPR guide RNA (gRNA), RNA interfering agent, oligonucleotide, peptide or peptidomimetic inhibitor, aptamer, antibody, or intrabody, optionally wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA);
c) comprises an antibody and/or intrabody, or an antigen binding fragment thereof, which specifically binds to the SS18-SSX fusion protein, the SSX tail, and/or the H2AK119Ub-marked nucleosome, optionally wherein the SSX tail is SSX tail (34 amino acid) and/or SSX tail (78 amino acid);
d) comprises an antibody and/or intrabody, or an antigen binding fragment thereof, which specifically binds to at least one of the following regions:
(1) the basic region of the SS18-SSX fusion protein;
(2) the acidic region of the SS18-SSX fusion protein;
(3) the acidic patch of the H2AK119Ub-marked nucleosome; and/or
(4) the H2AK119Ub mark,
further optionally wherein the antibody and/or intrabody, or antigen binding fragment thereof, i) is chimeric, humanized, composite, or human, and/or ii) comprises an effector domain, comprises an Fc domain, and/or is selected from the group consisting of Fv, Fav, F(ab′)2, Fab′, dsFv, scFv, sc(Fv)2, and diabodies fragments;
e) induces deletion or mutation of the basic region of the SS18-SSX fusion protein, the acidic region of the SS18-SSX fusion protein, the acidic patch of the H2AK119Ub-marked nucleosome, and/or a region within the SSX tail (34 amino acid);
f) inhibits H2A ubiquitination;
g) inhibits ubiquitin ligase activity of a PRC1 complex;
h) reduces expression, copy number, and/or ubiquitin ligase activity of RING1A and/or RING1B;
i) inhibits recruitment of a SS18-SSX fusion protein-bound BAF complex to an H2AK119Ub-marked nucleosome;
j) inhibits activation of at least one oncogenic target gene of the SS18-SSX fusion protein, optionally wherein the oncogenic target gene of the SS18-SSX fusion protein is selected from the group consisting of WNT16 and oncogenic target genes listed in McBride et al. (2018) Cancer Cell 33:1128-1141;
k) reduces the number of viable or proliferating cells in the cancer, and/or reduces the volume or size of a tumor comprising the cancer cells; and/or
l) is administered in a pharmaceutically acceptable formulation.

5-18. (canceled)

19. The method of claim 1, further comprising administering to the subject il an immunotherapy and/or cancer therapy and/or 2) at least one additional therapeutic agent or regimen for treating the cancer, optionally wherein
a) the immunotherapy and/or cancer therapy is administered before, after, or concurrently with the agent,
b) the immunotherapy is cell-based;
c) the immunotherapy comprises a cancer vaccine and/or virus;
d) the immunotherapy inhibits an immune checkpoint, optionally wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; and/or
e) the cancer therapy is selected from the group consisting of radiation, a radiosensitizer, and a chemotherapy.

20-25. (canceled)

26. A method of reducing viability or proliferation of synovial sarcoma cells comprising contacting the synovial sarcoma cells with an agent that inhibits binding of a SS18-SSX fusion protein according to claim 1 to a nucleosome, optionally wherein the nucleosome is an H2AK119Ub-marked nucleosome and/or the step of administering or contacting occurs in vivo, ex vivo, or in vitro.

27-28. (canceled)

29. The method of claim 26, wherein the agent is as according to claim 4.

30-42. (canceled)

43. The method of claim 26, further comprising contacting the cancer cells with an immunotherapy and/or cancer therapy, optionally wherein
a) the immunotherapy and/or cancer therapy is administered before, after, or concurrently with the agent,
b) the immunotherapy is cell-based;
c) the immunotherapy comprises a cancer vaccine and/or virus;
d) the immunotherapy inhibits an immune checkpoint, optionally wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; and/or
e) the cancer therapy is selected from the group consisting of radiation, a radiosensitizer, and a chemotherapy.

44-48. (canceled)

49. A method of assessing the efficacy of the agent according to claim 26 for treating synovial sarcoma in a subject, comprising;
a) detecting in a subject sample at a first point in time the number of viable and/or proliferating cancer cells;
b) repeating step a) during at least one subsequent point in time after administration of the agent; and
c) comparing number of viable and/or proliferating cancer cells detected in steps a) and b), wherein the absence of, or a significant decrease in number of viable and/or proliferating cancer cells in the subsequent sample as compared to the amount in the sample at the first point in time, indicates that the agent treats synovial sarcoma in the subject.

50. The method of claim 49, wherein
a) between the first point in time and the subsequent point in time, the subject has undergone treatment, completed treatment, and/or is in remission for synovial sarcoma;
b) the first and/or at least one subsequent sample is selected from the group consisting of ex vivo and in vivo samples;
c) the first and/or at least one subsequent sample is obtained from an animal model of synovial sarcoma;
d) the first and/or at least one subsequent sample is a portion of a single sample or pooled samples obtained from the subject;
e) the sample comprises cells, serum, peritumoral tissue, and/or intratumoral tissue obtained from the subject;
f) the method further comprises determining responsiveness to the agent by measuring at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

51-57. (canceled)

58. A cell-based assay for screening for agents that reduce viability or proliferation of a synovial sarcoma cell comprising;
a) contacting the synovial sarcoma cell with a test agent; and
b) determining the ability of the test agent to inhibit binding of a SS18-SSX fusion protein according to claim 1, a SSX (78 amino acid) region, and/or a SSX (34 amino acid) minimal region to a nucleosome, optionally wherein the nucleosome is a H2AK119Ub-marked nucleosome and/or the step of contacting occurs in vivo, ex vivo, or in vitro.

59-61. (canceled)

62. The cell-based assay of claim 58, further comprising
a) determining the ability of the test agent to inhibit recruitment of a SS18-SSX fusion protein-bound BAF complex to an H2AK119Ub-marked nucleosome and/or H2AK 119Ub-marked region of chromatin in cells, optionally wherein the cellular chromatin comprises a PRC1/H2A Ub domain;
b) determining the ability of the test agent to inhibit activation of at least one oncogenic target gene of the SS18-SSX fusion protein, optionally wherein the oncogenic target gene of the SS18-SSX fusion protein is selected from the group consisting of WNT16 and oncogenic target genes listed in McBride et al. (2018) Cancer Cell 33:1128-1141; and/or
c) determining a reduction in the viability or proliferation of the cancer cells.

63-65. (canceled)

66. An in vitro assay for screening for agents that reduce viability or proliferation of a synovial sarcoma cell comprising;
a) mixing a protein comprising a c-terminal basic region and a c-terminal acidic region of a SSX protein and a nucleosome together, optionally wherein the nucleosome is a H2AK119Ub-marked nucleosome;
b) adding a test agent to the mixture; and
c) determining the ability of the test agent to decrease binding of the protein to the nucleosome.

67. The in vitro assay of claim 66, wherein the protein
a) comprises c-terminal 34 amino acids (aa155-188) of a SSX protein, optionally wherein the SS18-SSX fusion protein is selected from Table 2; and/or
b) comprises c-terminal 78 amino acids (aa 111-188) of a SSX protein, optionally wherein the SS18-SSX fusion protein is selected from Table 2.

68-81. (canceled)

82. An isolated modified protein complex selected from the group consisting of protein complexes listed in Table 3, wherein the isolated modified protein complex comprises at least one subunit that is modified, optionally wherein the at least one modified subunit is a fragment of the subunit.

83. (canceled)

84. The isolated modified protein complex of claim 82, wherein the fragment of the subunit
a) binds to at least one binding partner of the subunit to form the isolated modified protein complex;
b) comprises the basic region and/or the acidic region of a SSX protein;
c) comprises c-terminal 34 amino acids (aa155-188) of a SSX protein;
d) comprises c-terminal 78 amino acids (aa 111-188) of a SSX protein; and/or
e) comprises the acidic patch of a nucleosome and/or the H2A K119 Ub mark.

85-87. (canceled)

88. The isolated modified protein complex of claim 82, wherein the SSX protein is selected form the group consisting of human SSX1, SSX2, SSX3, SSX4, SSX6, SSX7, SSX8, and SSX9.

89. (canceled)

90. The isolated modified protein complex of claim 82, wherein at least one subunit
a) is linked to at least another subunit;
b) is linked to at least another subunit through covalent cross-links;
c) is linked to at least another subunit through a peptide linker;
d) comprises a heterologous amino acid sequence; and/or
e) is selected from the group consisting of HA-SS18-SSX1, V5-SS18-SSX1, V5-SS18-SSX1 34aa tail, V5-SS18-SSX1 78aa tail, H2A, and H2B.

91-93. (canceled)

94. The isolated modified protein complex of claim 90, wherein the heterologous amino acid sequence comprises an affinity tag or a label.

95. The isolated modified protein complex of claim 94, wherein the affinity tag is selected from the group consisting of Glutathione-S-Transferase (GST), calmodulin binding protein (CBP), protein C tag, Myc tag, HaloTag, HA tag, Flag tag, His tag, biotin tag, and V5 tag.

96. The isolated modified protein complex of claim 95, wherein the label is a fluorescent protein.

97. (canceled)

98. A pharmaceutical composition comprising the isolated modified protein complex according to claim 82 and a carrier.
